(19) United States
(12) Patent Application Publication
(10) Pub. No.: US 2022/0324835 A1
(43) Pub. Date: Oct. 13, 2022

(54) INHIBITORS OF CBL-B AND METHODS OF USE THEREOF

(71) Applicant: Nurix Therapeutics, Inc., San Francisco, CA (US)

(72) Inventors: Paul A. BARSANTI, San Francisco, CA (US); Neil F. BENCE, San Francisco, CA (US); Jennifa GOSLING, San Francisco, CA (US); Anjanabha SAHA, San Francisco, CA (US); Asad M TAHERBHOY, San Francisco, CA (US); Christoph W. ZAPF, San Francisco, CA (US); Kathleen BOYLE, San Francisco, CA (US); Mario CARDOZO, San Francisco, CA (US); Jeffrey MIHALIC, San Francisco, CA (US); Morgan LAWRENZ, San Francisco, CA (US); Mark GALLOP, San Francisco, CA (US); Jilliane BRUFFEY, San Francisco, CA (US); Thomas CUMMINS, San Francisco, CA (US); Daniel ROBBINS, San Francisco, CA (US); Hiroko TANAKA, San Francisco, CA (US); Chenbo WANG, San Francisco, CA (US); Frederick COHEN, San Francisco, CA (US); Wylie PALMER, San Francisco, CA (US); Arthur T. SANDS, San Francisco, CA (US); Hunter SHUNATONA, San Francisco, CA (US)

(21) Appl. No.: 16/964,979

(22) PCT Filed: Jan. 25, 2019
(86) PCT No.: PCT/US19/15250

§ 371 Date: Jul. 25, 2020

Related U.S. Application Data
(60) Provisional application No. 62/622,667, filed on Jan. 26, 2018.
(60) Provisional application No. 62/693,359, filed on Jul. 2, 2018.

(51) Int. Cl. C07D 401/14; A61K 35/17; A61K 39/00; C07D 249/12; A61K 31/4196; C07D 401/12; A61K 31/455; C07D 403/12; A61K 31/506; A61K 31/4725; A61K 31/4709; A61K 31/497; A61K 31/4375; C07D 471/04; A61K 31/427; C07D 417/12; C07D 405/14; A61K 31/428; C07D 413/14; A61K 31/423; A61K 31/4545; A61K 31/496; C07D 413/12; C07D 491/107; C07D 413/10; A61K 31/4245; A61K 31/4155; A61K 31/4178; C07D 403/10; C07D 487/04; A61K 31/433; C07D 491/052; C07D 451/02; A61K 31/55; C07D 471/08; A61K 31/5377; A61K 31/541; C07D 403/14; C07D 417/14; A61K 31/438; A61K 31/499; A61K 31/4985; C07D 491/08; A61K 31/436; A61K 31/4748; A61K 31/437; C07D 498/04; A61K 31/5386; C07D 498/10; A61P 35/00; A61K 39/39; C07D 453/02; C12N 5/0783; C12N 5/0781

(52) U.S. Cl. CPC C07D 401/14; C12N 2501/599; A61K 39/0011; C07D 249/12; A61K 31/4196; C07D 401/12; A61K 31/455; C07D 403/12; A61K 31/506; A61K 31/4725; A61K 31/4709; A61K 31/497; A61K 31/4375; C07D 471/04; A61K 31/427; C07D 417/12; C07D 405/14; A61K 31/428; C07D 413/14; A61K 31/423; A61K 31/4545; A61K 31/496; C07D 413/12; C07D 491/107; C07D 413/10; A61K 31/4245; A61K 31/4155; A61K 31/4178; C07D 403/10; C07D 487/04; A61K 31/433; C07D 491/052; C07D 451/02; A61K 31/55; C07D 471/08; A61K 31/5377; A61K 31/541; C07D 403/14; C07D 417/14; A61K 31/438; A61K 31/499; A61K 31/4985; C07D 491/08; A61K 31/436; A61K 31/4748; A61K 31/437; C07D 498/04; A61K 31/5386; C07D 498/10; A61P 35/00; A61K 39/39; C07D 453/02; C12N 5/0636; C12N 5/0646; C12N 5/0635; A61K 2039/5158; C12N 2501/72; C12N 2501/2302; C12N 2501/515; A61K 35/17

(57) ABSTRACT

Compounds, compositions, and methods for use in inhibiting the E3 enzyme Cbl-b in the ubiquitin proteasome pathway are disclosed. The compounds, compositions, and methods can be used to modulate the immune system, to treat diseases amenable to immune system modulation, and for treatment of cells in vivo, in vitro, or ex vivo.

### CROSS-REFERENCE TO RELATED APPLICATIONS

This application claims priority to U.S. Provisional Application No. 62/622,667, filed Jan. 26, 2018, and U.S. Provisional Application No. 62/693,359, filed Jul. 2, 2018, the contents of which are hereby incorporated by reference in their entirety for all purposes.

### TECHNICAL FIELD OF THE INVENTION

Provided herein are compounds and compositions for inhibition of the Cbl-b enzyme and methods of use thereof in modulating the immune system, treatment of diseases, and treatment of cells in vivo, in vitro, or ex vivo.

### BACKGROUND

The ubiquitin proteasome pathway is a complex system involved in the regulation of protein function and catabolism. Proteins in eukaryotic cells are conjugated with ubiquitin, a 76 amino acid, 8.5 kilodalton protein. This conjugation, known as ubiquitination, results in altered function or degradation of the target protein. Ubiquitination of the target protein occurs via a coupled series of reactions involving ubiquitin and a set of enzymes known as E1, E2, and E3 enzymes. Ubiquitin is activated by the ubiquitin-activating enzyme, or E1 enzyme. Ubiquitin is then transferred to a ubiquitin-conjugating enzyme, or E2 enzyme. Finally, a ubiquitin ligase, or E3 enzyme, promotes the transfer of ubiquitin from the E2 enzyme to the target protein. Polyubiquitination of the target protein predominantly serves as a signal leading to degradation of the ubiquitin-conjugated protein by the proteasome, where it undergoes proteolysis. Ubiqutination by E3 ligases can also result in altered protein activity, interactions, or localization. Ubiquitination regulates diverse biology including cell division, DNA repair, and cellular signaling.

The synthesis and degradation of proteins in the cell is critical for cell cycle regulation, cell proliferation, apoptosis, and many other cellular processes. Thus, the ability to modulate the ubiquitin proteasome pathway offers a wealth of opportunities to intervene in disease processes. Mechanisms for intervention can include enhanced degradation of oncogene products, reduced degradation of tumor-suppressor proteins, and modulation of immune cell response.

Approximately 35 E2 enzymes and over 500 E3 enzymes are encoded in the human genome. Discovery of agents that modulate E2 or E3 enzymes accordingly provides the potential for therapies directed against disease processes involving a particular E2 or E3 enzyme. The present patent application is directed to agents that inhibit one such E3 enzyme, Casitas B-lineage lymphoma proto-oncogene-b (Cbl-b).

### SUMMARY OF THE INVENTION

Provided herein are compounds and pharmaceutical compositions as described herein for inhibition of Cbl-b, as well as methods for modulating the immune system using the compounds and pharmaceutical compositions, including enhancing an immune response using the compounds and pharmaceutical compositions. The compounds and compositions, and the methods of modulating the immune system, can be used in treating various diseases and disorders. The compounds and compositions can also be used for treatment of cells in vivo, in vitro, or ex vivo. Cells treated with compounds and compositions as disclosed herein can also be used in treating various diseases and disorders.

In one embodiment, provided herein are compounds of Formula (I-A):

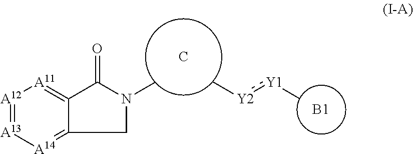

or a tautomer thereof, or a pharmaceutically acceptable salt of any of the foregoing, wherein

A¹¹ is CR¹¹ or N,

A¹² is CR¹² or N,

A¹³ is CR¹³ or N, and

A¹⁴ is CR¹⁴ or N,

wherein no more than two of A¹¹, A¹², A¹³, and A¹⁴ are N;

R¹¹, R¹², R¹³, and R¹⁴ are independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{j}R^{k} where R^{j} and R^{k} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{k} can be additionally chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{j} and R^{k} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a —C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring,

—(C₁-C₄ alkylene)-NR^{l}R^{m}, —O—(C₁-C₄ alkylene)-C(═O)NR^{l}R^{m}, or —O—(C₁-C₄ alkylene)-C(═O)NR^{l}R^{m}, wherein R^{l} and R^{m} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{l} and R^{m} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH,

—S(═O)₂—C₁-C₈ alkyl,

—SF₅, and

—S(═O)₂NRⁿR^{o} wherein Rⁿ and R^{o} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or Rⁿ and R^{o} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

Ring C,

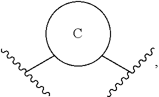

is selected from the group consisting of:

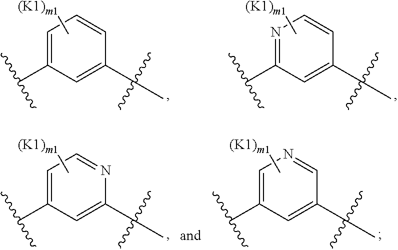

each K1 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —C₁-C₈ alkyl, —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO-(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K1 groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —C₁-C₈ haloalkyl, —O—C₁-C₈ alkyl, and —NR^{g1}R^{h1}, where R^{g1} and R^{h1} are independently H or C₁-C₈ alkyl;

m1 is 0, 1, or 2;

[symbol] is a single bond or a double bond,

wherein when [symbol] is a single bond,

Y1 is C(R¹⁹)(R²⁰), S, or O; and

Y2 is C(R¹⁷)(R¹⁸), and

when [symbol] is a double bond,

Y1 is C(R¹⁹); and

Y2 is C(R¹⁸),

R¹⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R¹⁸ is selected from the group consisting of H, F, —OH, C₁-C₈ alkyl optionally substituted with —OH, halogen or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl, or

when Y1 is C(R¹⁹)(R²⁰) or C(R¹⁹), R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl, or

R¹⁷ and R¹⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or C₁-C₈ alkylene-OH;

R¹⁹ and R²⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R¹⁹ can be taken together with R¹⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; or

R¹⁹ and R²⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and

Ring B1,

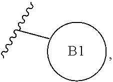

is a five-membered heteroaryl ring containing at least one N, O, or S ring atom, wherein Ring B1 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In one embodiment, provided herein are compounds of Formula (I):

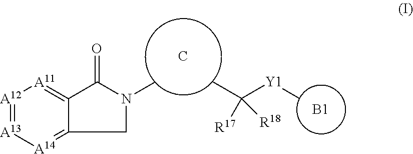

or tautomers thereof, or pharmaceutically acceptable salts of any of the foregoing, wherein

A¹¹ is CR¹¹ or N,

A¹² is CR¹² or N,

A¹³ is CR¹³ or N, and

A¹⁴ is CR¹⁴ or N,

wherein no more than two of A¹¹, A¹², A¹³, and A¹⁴ are N;

R¹¹, R¹², R¹³, and R¹⁴ are independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{j}R^{k} where R^{j} and R^{k} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{k} can be additionally chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{j} and R^{k} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, and —CN, —C(═O)OH, and —S(═O)₂—C₁-C₈ alkyl, —(C₁-C₄ alkylene)-NR^{l}R^{m}, —O—(C₁-C₄ alkylene)-NR^{l}R^{m}, —C(═O)NR^{l}R^{m}, —(C₁-C₄ alkylene)-C(═O)NR^{l}R^{m}, or —O—(C₁-C₄ alkylene)-C(═O)NR^{l}R^{m}, wherein R^{l} and R^{m} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{l} and R^{m} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH, —S(═O)₂—C₁-C₈ alkyl,

—SF₅, and

—S(═O)₂NRⁿR^{o} wherein Rⁿ and R^{o} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or Rⁿ and R^{o} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

Ring C,

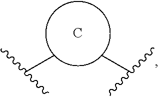

is selected from the group consisting of:

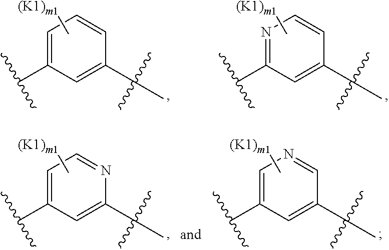

each K1 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K1 groups are taken together with the atoms to which they are attached to form a three- to six-membered carbocyclic or heterocyclic ring, a phenyl ring, or a five- to six-membered heteroaryl ring, wherein the ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —C₁-C₈ haloalkyl, —O—C₁-C₈ alkyl, and —NR^{g1}R^{h1}, where R^{g1} and R^{h1} are independently H or C₁-C₈ alkyl;

m1 is 0, 1, or 2;

R¹⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R¹⁸ is selected from the group consisting of H, F, —OH, C₁-C₈ alkyl optionally substituted with —OH, halogen or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl, or

when Y1 is C(R¹⁹)(R²⁰), R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl, or

R¹⁷ and R¹⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

Y1 is C(R¹⁹)(R²⁰) or S;

R¹⁹ and R²⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R¹⁹ can be taken together with R¹⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl;

or

R¹⁹ and R²⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and

Ring B1,

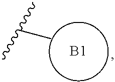

is a five-membered heteroaryl ring containing at least one N, O, or S ring atom, wherein Ring B1 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In some embodiments of the compounds of Formula (I-A), (I), or tautomers thereof, or pharmaceutically acceptable salts of any of the foregoing,

A¹¹ is CR¹¹ or N,

A¹² is CR¹² or N,

A¹³ is CR¹³ or N, and

A¹⁴ is CR¹⁴ or N,

wherein no more than two of A¹¹, A¹², A¹³, and A¹⁴ are N;

R¹¹, R¹², R¹³, and R¹⁴ are independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{j}R^{k} where R^{j} and R^{k} are independently H, C₁-C₈ alkyl, C₁-C₈ haloalkyl, or C₃-C₈ cycloalkyl, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{k} can be additionally chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{j} and R^{k} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, or I, —(C₁-C₄ alkylene)-NR^{l}R^{m}, —O—(C₁-C₄ alkylene)-NR^{l}R^{m}, —C(═O)NR^{l}R^{m}, —(C₁-C₄ alkylene)-C(═O)NR^{l}R^{m}, or —O—(C₁-C₄ alkylene)-C(═O)NR^{l}R^{m}, wherein R^{l} and R^{m} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{l} and R^{m} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH,

—S(═O)₂—C₁-C₈ alkyl,

—SF₅, and

—S(═O)₂NRⁿR^{o} wherein Rⁿ and R^{o} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or Rⁿ and R^{o} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

Ring C,

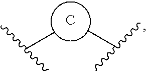

is selected from the group consisting of:

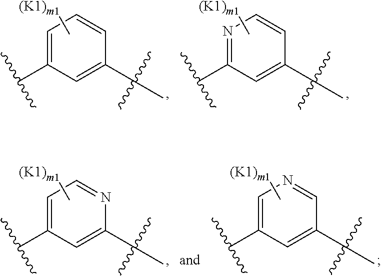

each K1 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl,

—O—C₁-C₈ haloalkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K1 groups are taken together with the atoms to which they are attached to form a three- to six-membered carbocyclic or heterocyclic ring, a phenyl ring, or a five- to six-membered heteroaryl ring, wherein the ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —C₁-C₈ haloalkyl, —O—C₁-C₈ alkyl, and —NR^{g1}R^{h1}, where R^{g1} and R^{h1} are independently H or C₁-C₈ alkyl;

m1 is 0, 1, or 2;

R¹⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R¹⁸ is selected from the group consisting of H, F, —OH, C₁-C₈ alkyl optionally substituted with —OH, halogen or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl, or

when Y1 is C(R¹⁹)(R²⁰), R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl, or

R¹⁷ and R¹⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl;

Y1 is C(R¹⁹)(R²⁰) or S;

R¹⁹ and R²⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R¹⁹ can be taken together with R¹⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; and

Ring B1,

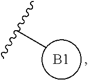

is a five-membered heteroaryl ring containing at least one N, O, or S ring atom, wherein Ring B1 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In another embodiment, provided herein are compounds of Formula (II-A):

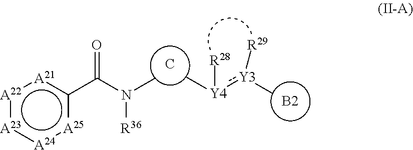

or a tautomer thereof, or a pharmaceutically acceptable salt of any of the foregoing,

wherein
A²¹ is CR²¹ or N, or is absent,
A²² is CR²² or N,
A²³ is CR²³ or N,
A²⁴ is CR²⁴ or N, and
A²⁵ is CR²⁵ or N,

wherein no more than two of A²¹, A²², A²³, A²⁴, and A²⁵ are N;

R²¹, R²², R²³, and R²⁴ are independently selected from R^{x};

each R^{x} is independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{p}R^{q} where R^{p} and R^{q} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{q} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{p} and R^{q} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a —C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{r}R^{s}, —O—(C₁-C₄ alkylene)-NR^{r}R^{s}, —C(═O)NR^{r}R^{s}, —(C₁-C₄ alkylene)-C(═O)NR^{r}R^{s}, or —O—(C₁-C₄ alkylene)-C(═O)NR^{r}R^{s}, wherein R^{r} and R^{s} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{r} and R^{s} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{t}R^{u} wherein R^{t} and R^{u} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{t} and R^{u} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R²⁵ is independently selected from R^{x}, and R²⁶ is H; or

A₂₅ is CR²⁵, and R²⁵, R²⁶ and the intervening atoms are taken together to form a five-membered lactam ring, such that the fragment

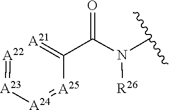

is

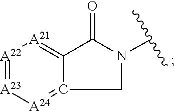

or

(R²¹ and R²²) or (R²² and R²³) or (R²³ and R²⁴) or (R²⁴ and R²⁵), together with the atoms to which they are attached, are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl);

Ring C,

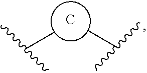

is selected from the group consisting of:

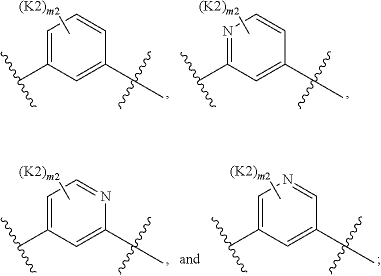

each K2 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —C₁-C₈ alkyl, —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO-(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K2 groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m2 is 0, 1, or 2;

[symbol] is a single bond or a double bond,

wherein when [symbol] is a single bond, Y3 is C(R³⁰) and Y4 is C(R²⁷); and

when [symbol] is a double bond, Y3 is C and Y4 is C;

R²⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl;

R²⁸ and R²⁹, as indicated by the dashed curve [symbol] are taken together with the atoms to which they are attached to form a three- to six-membered cycloalkyl, heterocyclyl, aryl, or heteroaryl ring, wherein the three- to six-membered cycloalkyl, heterocyclyl, aryl, or heteroaryl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl,

R³⁰ is selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; and

Ring B2,

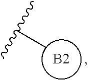

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In another embodiment, provided herein are compounds of Formula (II):

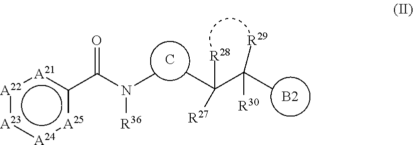

or tautomers thereof, or pharmaceutically acceptable salts of any of the foregoing, wherein

A²¹ is CR²¹ or N, or is absent,

A²² is CR²² or N,

A²³ is CR²³ or N,

A²⁴ is CR²⁴ or N, and

A²⁵ is CR²⁵ or N,

wherein no more than two of A²¹, A²², A²³, A²⁴, and A²⁵ are N;

R²¹, R²², R²³, and R²⁴ are independently selected from R^{x};

each R^{x} is independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{p}R^{q} where R^{p} and R^{q} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{q} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{p} and R^{q} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, CN, —C(═O)OH, and —S(═O)₂—Cᵣ C₈ alkyl, —(C₁-C₄ alkylene)-NR^{r}R^{s}, —O—(C₁-C₄ alkylene)-NR^{r}R^{s}, —C(═O)NR^{r}R^{s}, —(C₁-C₄ alkylene)-C(═O)NR^{r}R^{s}, or —O—(C₁-C₄ alkylene)-C(═O)NR^{r}R^{s}, wherein R^{r} and R^{s} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{r} and R^{s} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{t}R^{u} wherein R^{t} and R^{u} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{t} and R^{u} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R²⁵ is independently selected from R^{x}, and R²⁶ is H; or

A²⁵ is CR²⁵, and R²⁵, R²⁶ and the intervening atoms are taken together to form a five-membered lactam ring, such that the fragment

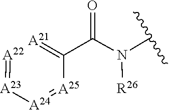

is

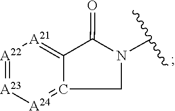

or

(R²¹ and R²²) or (R²² and R²³) or (R²³ and R²⁴) or (R²⁴ and R²⁵), together with the atoms to which they are attached, are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl);

Ring C,

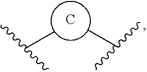

is selected from the group consisting of:

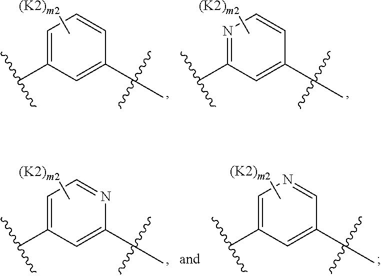

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K2 groups are taken together with the atoms to which they are attached to form a three- to six-membered carbocyclic or heterocyclic ring, a phenyl ring, or a five- to six-membered heteroaryl ring, wherein the ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m2 is 0, 1, or 2;

R²⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl;

R²⁸ and R²⁹, as indicated by the dashed curve [symbol] , are taken together with the atoms to which they are attached to form a three- to six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl,

R³⁰ is selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; and

Ring B2,

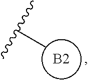

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In some embodiments of the compounds of Formula (II-A), (II), or tautomers thereof, or pharmaceutically acceptable salts of any of the foregoing,

A²¹ is CR²¹ or N, or is absent,

A²² is CR²² or N,

A²³ is CR²³ or N,

A²⁴ is CR²⁴ or N, and

A²⁵ is CR²⁵ or N,

wherein no more than two of A²¹, A²², A²³, A²⁴, and A²⁵ are N;

R²¹, R²², R²³, and R²⁴ are independently selected from R^{x};

each R^{x} is independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{p}R^{q} where R^{p} and R^{q} are independently H, C₁-C₈ alkyl, C₁-C₈ haloalkyl, or C₃-C₈ cycloalkyl, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{q} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{p} and R^{q} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, or I, —(C₁-C₄ alkylene)-NR^{r}R^{s}, —O—(C₁-C₄ alkylene)-NR^{r}R^{s}, —C(═O)NR^{r}R^{s}, —(C₁-C₄ alkylene)-C(═O)NR^{r}R^{s}, or —O—(C₁-C₄ alkylene)-C(═O)NR^{r}R^{s}, wherein R^{r} and R^{s} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{r} and R^{s} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{t}R^{u} wherein R and R^{u} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{t} and R^{u} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R²⁵ is independently selected from R^{x}, and R²⁶ is H; or

A₂₅ is CR²⁵, and R²⁵, R²⁶ and the intervening atoms are taken together to form a five-membered lactam ring, such that the fragment

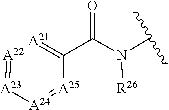

is

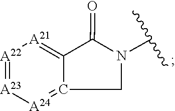

or

(R²¹ and R²²) or (R²² and R²³) or (R²³ and R²⁴) or (R²⁴ and R²⁵), together with the atoms to which they are attached, are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl);

Ring C,

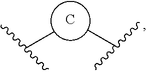

is selected from the group consisting of:

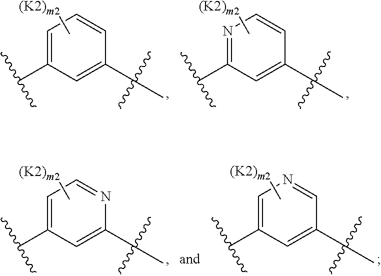

each K2 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl,

—O—C₁-C₈ haloalkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K2 groups are taken together with the atoms to which they are attached to form a three- to six-membered carbocyclic or heterocyclic ring, a phenyl ring, or a five- to six-membered heteroaryl ring, wherein the ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m2 is 0, 1, or 2;

R²⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl;

R²⁸ and R²⁹, as indicated by the dashed curve [symbol] , are taken together with the atoms to which they are attached to form a three- to six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl,

R³⁰ is selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; and

Ring B2,

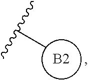

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In another embodiment, provided herein are pharmaceutical compositions comprising a Cbl-b inhibitor of Formula (III-A):

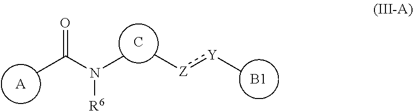

or a tautomer thereof, or a pharmaceutically acceptable salt of any of the foregoing, and a pharmaceutically acceptable excipient;

wherein Ring A,

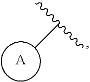

is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;

wherein Ring A is attached to the adjacent carbonyl carbon via a carbon atom of Ring A;

each R^{A} is independently selected from the group consisting of:

H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R⁶ is H, or

R⁶ and the amide group to which R⁶ is connected attach to Ring A to form a five-membered lactam ring fused to Ring A, such that the fragment

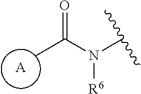

is

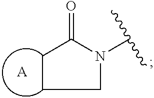

Ring C,

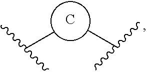

is selected from the group consisting of:

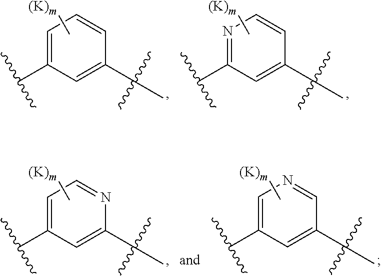

each K is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —C₁-C₈ alkyl, —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO-(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m is 0, 1, or 2;

[symbol] is a single bond or a double bond,

wherein when [symbol] is a single bond,

Y is C(R⁹)(R¹⁰), S, or O; and

Z is C(R⁷)(R⁸), and

when [symbol] is a double bond,

Y is C(R⁹); and

Z is C(R⁸),

R⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R⁸ is selected from the group consisting of H, F, —OH, C₁-C₈ alkyl optionally substituted with —OH or —O—C₁-C₈ alkyl, C₂-C₈ alkenyl, C₁-C₈ haloalkyl, and —O—C₁-C₈ alkyl optionally substituted with —OH or —O—C₁-C₈ alkyl, or

when Y is C(R⁹)(R¹⁰) or C(R⁹), R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl, or

R⁷ and R⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

R⁹ and R¹⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R⁹ is taken together with R⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; or

R⁹ and R¹⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and

Ring B,

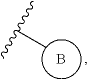

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In another embodiment, provided herein are pharmaceutical compositions comprising a Cbl-b inhibitor of Formula (III):

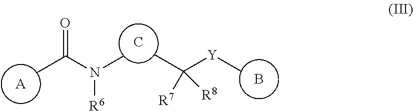

or a tautomer thereof, or a pharmaceutically acceptable salt of any of the foregoing, and a pharmaceutically acceptable excipient;

wherein

Ring A,

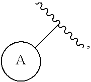

is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;

wherein Ring A is attached to the adjacent carbonyl carbon via a carbon atom of Ring A;

each R^{A} is independently selected from the group consisting of:

H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl, —(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R⁶ is H, or

R⁶ and the amide group to which R⁶ is connected attach to Ring A to form a five-membered lactam ring fused to Ring A, such that the fragment

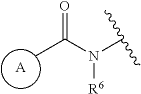

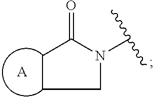

Ring C,

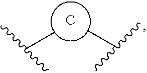

is selected from the group consisting of:

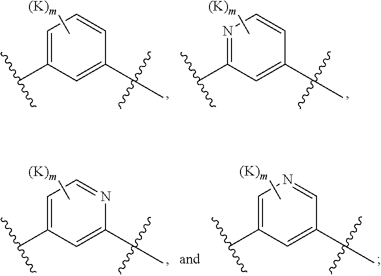

each K is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m is 0, 1, or 2;

R⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R⁸ is selected from the group consisting of H, F, —OH, C₁-C₈ alkyl optionally substituted with —OH or —O—C₁-C₈ alkyl, C₂-C₈ alkenyl, C₁-C₈ haloalkyl, and —O—C₁-C₈ alkyl optionally substituted with —OH or —O—C₁-C₈ alkyl, or

when Y is C(R⁹)(R¹⁰), R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl, or

R⁷ and R⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

Y is C(R⁹)(R¹⁰) or S;

R⁹ and R¹⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R⁹ is taken together with R⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl;

or

R⁹ and R¹⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and

Ring B,

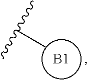

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In some embodiments of the Cbl-b inhibitor of Formula (III-A), (III),

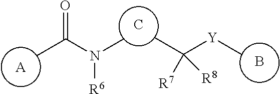

or a tautomer thereof, or a pharmaceutically acceptable salt of any of the foregoing,

wherein Ring A,

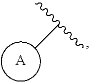

is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;

wherein Ring A is attached to the adjacent carbonyl carbon via a carbon atom of Ring A;

each R^{A} is independently selected from the group consisting of:

H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{a}R^{b} where R^{a} and R^{b} are independently H, C₁-C₈ alkyl, C₁-C₈ haloalkyl, or C₃-C₈ cycloalkyl, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, or I, —(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R⁶ is H, or

R⁶ and the amide group to which R⁶ is connected attach to Ring A to form a five-membered lactam ring fused to Ring A, such that the fragment

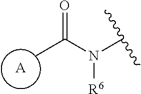

is

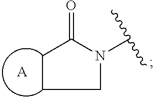

Ring C,

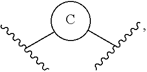

is selected from the group consisting of:

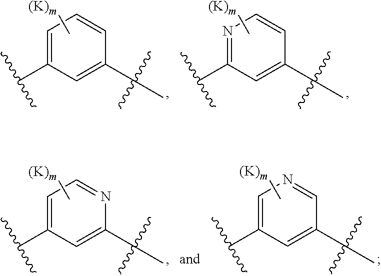

each K is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl,

—O—C₁-C₈ haloalkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m is 0, 1, or 2;

R⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R⁸ is selected from the group consisting of H, F, —OH, C₁-C₈ alkyl optionally substituted with —OH or —O—C₁-C₈ alkyl, C₂-C₈ alkenyl, C₁-C₈ haloalkyl, and —O—C₁-C₈ alkyl optionally substituted with —OH or —O—C₁-C₈ alkyl, or

when Y is C(R⁹)(R¹⁰), R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl, or

R⁷ and R⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl;

Y is C(R⁹)(R¹⁰) or S;

R⁹ and R¹⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R⁹ is taken together with R⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; and

Ring B,

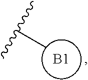

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In another embodiment, provided herein are pharmaceutical compositions comprising a Cbl-b inhibitor of Formula (IV):

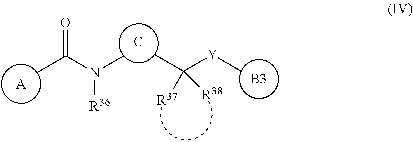

or a tautomer thereof, or a pharmaceutically acceptable salt of any of the foregoing, and a pharmaceutically acceptable excipient; wherein

Ring A,

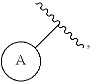

is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;

wherein Ring A is attached to the adjacent carbonyl carbon via a carbon atom of Ring A;

each R^{A} is independently selected from the group consisting of: H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a —C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R³⁶ is H, or

R³⁶ and the amide group to which R³⁶ is connected attach to Ring A to form a five-membered lactam ring fused to Ring A, such that the fragment

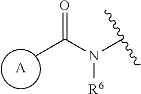

is

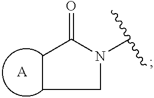

Ring C,

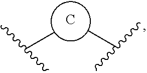

is selected from the group consisting of:

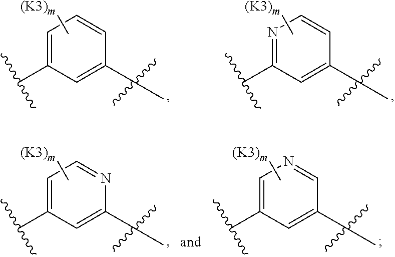

each K3 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —C₁-C₈ alkyl, —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO-(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m is 0, 1, or 2;

R³⁷ and R³⁸ together with the carbon to which they are attached form a C₃-C₈ cycloalkyl ring or three- to six-membered heterocyclyl ring as indicated by the dashed curve [symbol] , wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

Y is C(R³⁹)(R⁴⁰) or S;

R³⁹ and R⁴⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; or

R³⁹ and R⁴⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

and

Ring B3,

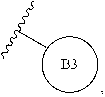

is a five-membered heteroaryl ring containing at least one N, O, or S ring atom, wherein Ring B3 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

In another embodiment, provided herein are compounds of Formula (IV-ox):

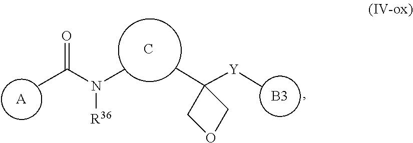

or a tautomer thereof, or a pharmaceutically acceptable salt of any of the foregoing, wherein R³⁷ and R³⁸ together with the carbon to which they are attached form an oxetane ring as shown, and where the remaining substituents are as in Formula (IV). Also disclosed are pharmaceutical compositions comprising a compound of Formula (IV-ox) and a pharmaceutically acceptable excipient.

In one aspect, provided herein are methods of modulating activity of an immune cell, the methods comprising contacting the immune cell with an effective amount of a Cbl-b inhibitor to modulate activity of the immune cell, wherein the Cbl-b inhibitor is a compound of Formula (I-A), Formula (I), Formula (II-A), Formula (II), Formula (III-A), Formula (III), Formula (IV), or Formula (IV-ox), or a tautomer thereof, or a pharmaceutically acceptable salt of any of the foregoing. In some embodiments, the immune cell is a T-cell, a B cell, or a natural killer (NK) cell. In some of any such embodiments, the immune cell is a T-cell, and modulating activity of the T-cell comprises one or more of increased T-cell activation, increased T-cell proliferation, decreased T-cell exhaustion, and decreased T-cell tolerance. In a further embodiment, increased T-cell activation comprises increased production of a cytokine such as one or more selected from the group consisting of: IL-2, IFN-γ, and TNFα. In yet another further embodiment, increased T-cell activation comprises increased cell surface expression of one or more T-cell activation markers such as one or both of CD25 and CD69. In some of any such embodiments, the T-cell has been or is in contact with an anti-CD3 antibody alone or in combination with an anti-CD28 antibody. In some of any such embodiments, the immune cell is a NK cell, and modulating activity of an NK cell comprises increased NK cell activation. In a further embodiment, increased NK cell activation comprises increased production of a cytokine such as IFN-γ. In some of any such embodiments, the immune cell is a B cell, and modulating activity of a B cell comprises increased B cell activation. In some any of such embodiments, the immune cell is a human immune cell. In some embodiments, the immune cell comprises a recombinant chimeric receptor such as a chimeric antigen receptor (CAR).

In another aspect, provided herein are methods of producing a modified immune cell, the methods comprising culturing a cell population containing an immune cell in the presence of an effective amount of a Cbl-b inhibitor to modulate the activity of the immune cell, thereby producing the modified immune cell, wherein the Cbl-b inhibitor is a compound of Formula (I-A), Formula (I), Formula (II-A), Formula (II), Formula (III-A), Formula (III), Formula (IV), or Formula (IV-ox), or a tautomer thereof, or a pharmaceutically acceptable salt of any of the foregoing. In some embodiments, the method further comprises the step of culturing the immune cell with an anti-CD3 antibody alone or in combination with an anti-CD28 antibody. In some of any such embodiments, the method further comprises the step of recovering the modified immune cell. In some of any such embodiments, the immune cell is a cell selected from the group consisting of: a hematopoietic cell, a multipotent stem cell, a myeloid progenitor cell, a lymphoid progenitor cell, a T-cell, a B cell, and a NK cell. In some of any such embodiments, the modified immune cell is a cell selected from the group consisting of: a hematopoietic cell, a multipotent stem cell, a myeloid progenitor cell, a lymphoid progenitor cell, a T-cell, a B cell, and a NK cell. In further embodiments, the immune cell is from an individual. In another further embodiment, the immune cell is a human immune cell. In some of any such embodiments, the immune cell or modified immune cell comprises a recombinant chimeric receptor such as a chimeric antigen receptor (CAR).

In another aspect, provided herein is a modified immune cell produced by a method provided herein.

In yet another aspect, provided herein is a modified immune cell comprising a Cbl-b inhibitor, wherein the Cbl-b inhibitor is a compound of Formula (I-A), Formula (I), Formula (II-A), Formula (II), Formula (III-A), Formula (III), Formula (IV), or Formula (IV-ox), or a tautomer thereof, or a pharmaceutically acceptable salt of any of the foregoing.

In one aspect, provided herein is an isolated modified immune cell, wherein the immune cell has been contacted or is in contact with a Cbl-b inhibitor, wherein the Cbl-b inhibitor is a compound of Formula (I-A), Formula (I), Formula (II-A), Formula (II), Formula (III-A), Formula (III), Formula (IV), or Formula (IV-ox), or a tautomer thereof, or a pharmaceutically acceptable salt of any of the foregoing. In some embodiments, the modified immune cell is a T-cell, a B cell, or a natural killer (NK) cell. In some of any such embodiments, the modified immune cell is a T-cell, and the T-cell exhibits one or more of increased T-cell activation, increased T-cell proliferation, decreased T-cell exhaustion, and decreased T-cell tolerance. In a further embodiment, increased T-cell activation comprises increased production of a cytokine such as one or more selected from the group consisting of: IL-2, IFN-γ, and TNFα. In yet another further embodiment, increased T-cell activation comprises increased cell surface expression of one or more T-cell activation markers such as one or both of CD25 and CD69. In some of any such embodiments, the T-cell has been or is in contact with an anti-CD3 antibody alone or in combination with an anti-CD28 antibody. In some of any such embodiments, the modified immune cell is a NK cell, and the NK cell exhibits increased NK cell activation. In a further embodiment, increased NK cell activation comprises increased production of a cytokine such as IFN-γ. In some of any such embodiments, the modified immune cell is a B cell, and the B cell exhibits increased B cell activation. In some any of such embodiments, the modified immune cell is a human immune cell. In some embodiments, the modified immune cell comprises a recombinant chimeric receptor such as a chimeric antigen receptor (CAR).

In another aspect, provided herein are compositions comprising a cell population containing a modified immune cell such as one of any of the embodiments herein, wherein the modified immune cell has been or is in contact with the Cbl-b inhibitor. In one embodiment, the modified immune cell has been or is in contact with an anti-CD3 antibody alone or in combination with an anti-CD28 antibody. In a further embodiment, the composition comprises a pharmaceutically acceptable excipient. In some of any such embodiments, the composition is in a culture vessel. In a further embodiment, the culture vessel is a tube, a dish, a bag, a multiwell plate or a flask. In some of any such embodiments, the composition is in a suitable container. In a further embodiment, the suitable container is a bottle, a vial, a syringe, an intravenous bag or a tube. In some embodiments, the modified immune cell comprises a recombinant chimeric receptor such as a CAR.

Also provided herein, in some aspects, are methods for modulating the immune response, wherein such methods comprise administering an effective amount of a modified immune cell provided herein or an effective amount of a composition provided herein to an individual in need thereof. In further embodiments, the individual has a cancer.

In some aspects, provided herein are methods for treating a cancer responsive to inhibition of Cbl-b activity, wherein such methods comprise administering an effective amount of a modified immune cell provided herein or an effective amount of a composition provided herein to an individual having the cancer responsive to inhibition of Cbl-b activity. In some embodiments, the cancer is a hematologic cancer or a non-hematologic cancer. In a further embodiment, the non-hematologic cancer is a sarcoma or a carcinoma. In another further embodiment, the hematologic cancer is a lymphoma, a leukemia or a myeloma.

In some aspects, provided herein are methods for inhibiting abnormal cell proliferation, wherein such methods comprise administering an effective amount of a modified immune cell provided herein or an effective amount of a composition provided herein to an individual in need thereof. In some embodiments, the abnormal cell proliferation is hyperplasia or cancer cell proliferation. In a further embodiment, the cancer cell is derived from a hematologic cancer or a non-hematologic cancer. In a further embodiment, the non-hematologic cancer is a sarcoma or a carcinoma. In another further embodiment, the hematologic cancer is a lymphoma, a leukemia or a myeloma.

Further provided herein, in some aspects, are methods for modulating the immune response, wherein such methods comprise administering an effective amount of a Cbl-b inhibitor to an individual to modulate the immune response in the individual, wherein the Cbl-b inhibitor is a compound of Formula (I-A), Formula (I), Formula (II-A), Formula (II), Formula (III-A), Formula (III), Formula (IV), or Formula (IV-ox), or a tautomer thereof, or a pharmaceutically acceptable salt of any of the foregoing.

In some aspects, provided are methods of inhibiting Cbl-b activity, wherein such methods comprise administering an effective amount of a Cbl-b inhibitor to an individual to inhibit Cbl-b activity in the individual, wherein the Cbl-b inhibitor is a compound of Formula (I-A), Formula (I), Formula (II-A), Formula (II), Formula (III-A), Formula (III), Formula (IV), or Formula (IV-ox), or a tautomer thereof, or a pharmaceutically acceptable salt of any of the foregoing.

In an other aspect, provided herein are methods of treating a cancer responsive to inhibition of Cbl-b activity, wherein such methods comprise administering an effective amount of a Cbl-b inhibitor to an individual to treat the cancer responsive to inhibition of Cbl-b activity, wherein the Cbl-b inhibitor is a compound of Formula (I-A), Formula (I), Formula (II-A), Formula (II), Formula (III-A), Formula (III), Formula (IV), or Formula (IV-ox), or a tautomer thereof, or a pharmaceutically acceptable salt of any of the foregoing. In some embodiments, the cancer is a hematologic cancer or a non-hematologic cancer. In a further embodiment, the non-hematologic cancer is a sarcoma or a carcinoma. In another further embodiment, the hematologic cancer is a lymphoma, a leukemia or a myeloma.

In some aspects, provided herein are methods of inhibiting abnormal cell proliferation, wherein such methods comprise administering an effective amount of a Cbl-b inhibitor to an individual to inhibit abnormal cell proliferation in the individual, wherein the Cbl-b inhibitor is a compound of Formula (I-A), Formula (I), Formula (II-A), Formula (II), Formula (III-A), Formula (III), Formula (IV), or Formula (IV-ox), or a tautomer thereof, or a pharmaceutically acceptable salt of any of the foregoing. In some embodiments, the abnormal cell proliferation is hyperplasia or cancer cell proliferation. In a further embodiment, the cancer cell is derived from a hematologic cancer or a non-hematologic cancer. In a further embodiment, the non-hematologic cancer is a sarcoma or a carcinoma. In another further embodiment, the hematologic cancer is a lymphoma, a leukemia or a myeloma.

In some aspects of the methods herein, an individual has one or more of increased T-cell activation, increased T-cell proliferation, decreased T-cell exhaustion, and decreased T-cell tolerance after administration of the Cbl-b inhibitor. In a further embodiment, increased T-cell activation comprises increased production of a cytokine such as one or more selected from the group consisting of: IL-2, IFN-γ, and TNFα. In some of any such embodiments, increased T-cell activation comprises increased cell surface expression of one or more T-cell activation markers such one or both of CD25 and CD69. In some of any such embodiments, the individual has increased NK cell activation after administration of the Cbl-b inhibitor. In a further embodiment, the increased NK cell activation comprises increased production of a cytokine such as IFN-γ. In some of any such embodiments, the individual has increased B cell activation after administration of the Cbl-b inhibitor.

Also provided are cell culture compositions comprising a cell population containing an immune cell and a Cbl-b inhibitor, wherein the Cbl-b inhibitor is a compound of Formula (I-A), Formula (I), Formula (II-A), Formula (II), Formula (III-A), Formula (III), Formula (IV), or Formula (IV-ox), or a tautomer thereof, or a pharmaceutically acceptable salt of any of the foregoing. In one embodiment, the immune cell is a cell selected from the group consisting of: a hematopoietic cell, a multipotent stem cell, a myeloid progenitor cell, a lymphoid progenitor cell, a T-cell, a B cell, and a NK cell. In a further embodiment, the cell culture composition further comprises an anti-CD3 antibody alone or in combination with an anti-CD28 antibody. In some of any such embodiments, the immune cell is an engineered immune cell comprising a recombinant chimeric receptor such as a CAR.

In yet another aspect, provided herein are articles of manufacture comprises a modified immune cell, a composition, a cell culture, and/or a pharmaceutical composition provided herein. In one embodiment, the modified immune cell or cell culture composition is in a tube, a dish, a bag, a multiwell plate or a flask. In another embodiment, the modified immune cell or pharmaceutical composition is in a bottle, a vial, a syringe, an intravenous bag or a tube.

In another aspect, provided herein are kits comprising a modified immune cell or a composition provided herein. In one embodiment, the modified immune cell or cell culture composition is in a tube, a dish, a bag, a multiwell plate or a flask. In another embodiment, the modified immune cell or pharmaceutical composition is in a bottle, a vial, a syringe, an intravenous bag or a tube. In some embodiments, the kit further comprises instructions for administering the modified immune cell or composition to an individual according to a method provided herein.

In some aspects, provided herein are kits comprising a pharmaceutical composition described herein. In some embodiments, the kit further comprises instructions for administering the pharmaceutical composition to an individual according to a method provided herein.

In one aspect, provided herein are kits comprising a cell culture composition provided herein. In some embodiments, the kit comprises instructions for producing a modified immune cell or a composition described herein.

### BRIEF DESCRIPTION OF THE DRAWINGS

FIG. 1A and FIG. 1B show a series of diagrams depicting the activity assay utilized to assay inhibition of Cbl-b activity by a Cbl-b inhibitor. FIG. 1A: The activity assay mixture comprises an N-terminal biotinylated Avi-tagged Cbl-b, an E2 enzyme (i.e., UbcH5B) irreversibly conjugated to Bodipy-Fluorescein tagged ubiquitin (i.e., UbcH5B-Ub), a Src kinase, streptavidin-terbium, and assay buffer. Phosphorylation of Cbl-b by the Src kinase allows UbcH5B-Ub to bind to Cbl-b and bring the Bodipy-Fluorescein tagged ubiquitin in proximity to the streptavidin-terbium bound to the N-terminal biotinylated Avi-tagged Cbl-b. The resulting complex produces a FRET signal indicating that Cbl-b is active or not subject to inhibition. FIG. 1B: In the presence of a Cbl-b inhibitor, such as one described herein, Cbl-b is unable to bind UbcH5B-Ub. The lack of a FRET signal indicates Cbl-b is inhibited by the Cbl-b inhibitor. Ub indicates ubiquitin; N-Avi indicates biotinylated Avi-tag at the N-terminus of Cbl-b; inhibitor indicates Cbl-b inhibitor.

### DETAILED DESCRIPTION

Provided herein are compounds and pharmaceutical compositions that inhibit the Cbl-b enzyme, as well as methods of treatment using such compounds and pharmaceutical compositions. The compounds and compositions can be used in methods of modulating the immune system, for treatment of diseases, and for treatment of cells in vivo, in vitro, or ex vivo.

T-cell activation and T-cell tolerance are tightly controlled processes regulating the immune response to tumors while preventing autoimmunity. Tolerance prevents the immune system from attacking cells expressing “self” antigens. During peripheral tolerance, T-cells that recognize “self” antigens (i.e., self-reactive T-cells) become functionally unresponsive or are deleted after encountering “self” antigens outside of the thymus. Peripheral tolerance processes therefore are important for preventing autoimmune diseases. Normally, cancer cells are removed by activated T-cells that recognize tumor antigens expressed on the surface of the cancer cells. However, in cancer, the tumor microenvironment can support T-cell tolerance to cancer cells, which allows cancer cells to avoid recognition and removal by the immune system. The ability of cancer cells to avoid tumor immunosurveillance can contribute to uncontrolled tumor growth. Therefore, T-cell tolerance can be a form of T-cell dysfunction. General principles of T-cell dysfunction are well known in the art. See Schietinger, A. et al., Trends Immunol., 35(2):51-60 (2014). Additional types of T-cell dysfunction that can contribute to uncontrolled tumor growth include T-cell exhaustion, T-cell senescence and/or T-cell anergy. Therefore, treating T-cell dysfunction, for example, by increasing T-cell activation, increasing T-cell proliferation, decreasing T-cell tolerance and/or decreasing T-cell exhaustion, is beneficial for preventing or treating cancer. Additional cells of the immune system are important for recognition and removal of cancer cells during immune surveillance. For example, Natural Killer (NK) cells are lymphocytes of the innate immune system that are able to identify and kill cancer cells. See Martinez-Losato, L. et al., Clin Cancer Res., 21(22):5048-5056 (2015). Recent studies have also shown that B-cell subsets with distinct phenotypes and functions exhibit diverse roles in the anti-tumor response. See Saravaria, A., et al., Cell Mol Immunol., 14(8):662-674 (2017). Due to their role in tumor surveillance, NK cells and B cells may also be amenable as therapeutic targets for the prevention or treatment of cancer.

Cbl-b is a RING-type E3 ligase that plays an important role in the immune system due to its function as a negative regulator of immune activation. Cbl-b has an essential role in decreasing the activation of T-cells, thereby enhancing T-cell tolerance. Studies have found that cbl-b-deficient T-cells display lower thresholds for activation by antigen recognition receptors and co-stimulatory molecules (e.g., CD28). For example, loss of Cbl-b in T-cells uncouples the requirement for CD28 costimulation during T-cell activation and proliferation. See Bachmaier, K. et al., Nature., 403(6766):211-216 (2000). Such cbl-b−/− T-cells are largely resistant to T-cell anergy, a tolerance mechanism in which T-cells are functionally inactivated and T-cell proliferation is greatly impaired. See Jeon, M et al., Immunity, 21(2): 167-177 (2004) and Schwartz et al., Annu Rev Immunol., 21:305-34 (2003). In support of this, loss of Cbl-b in cbl-b knockout mice resulted in impaired induction of T-cell tolerance and exacerbated autoimmunity. See Jeon, M et al., Immunity, 21(2): 167-177 (2004). Importantly, loss of Cbl-b in mice also resulted in a robust anti-tumor response that depends primarily on cytotoxic T cells. One study showed that cbl-b−/−CD8+ T cells are resistant to T regulatory cell-mediated suppression and exhibit enhanced activation and tumor infiltration. Therapeutic transfer of naive cbl-b−/−CD8+ T cells was sufficient to mediate rejection of established tumors. See Loeser, S. et al., J Exp Med., 204(4):879-891 (2007). Recent studies have shown that Cbl-b also plays a role in NK cell activation. Genetic deletion of Cbl-b or targeted inactivation of its E3 ligase activity allowed NK cells to spontaneously reject metastatic tumors in a mouse model. See Paolino, M. et al., Nature, 507(7493):508-512.

Provided herein are compounds and compositions that inhibit Cbl-b and can be used in novel approaches to treat diseases such as cancer. In some embodiments, the compounds and compositions provided herein can be used in methods of modulating the immune system, such as increasing activation of T-cells, NK cells and B cells as well as in the treatment of such cells in vivo, in vitro, or ex vivo.

### I. Definitions

An “effective amount” of an agent disclosed herein is an amount sufficient to carry out a specifically stated purpose. An “effective amount” may be determined empirically and in a routine manner, in relation to the stated purpose. An “effective amount” or an “amount sufficient” of an agent is that amount adequate to produce a desired biological effect, such as a beneficial result, including a beneficial clinical result. In some embodiments, the term “effective amount” refers to an amount of an agent effective to “treat” a disease or disorder in an individual (e.g., a mammal such as a human).

The term “Cbl-b” as used herein refers to a Cbl-b protein. The term also includes naturally occurring variants of Cbl-b, including splice variants or allelic variants. The term also includes non-naturally occurring variants of Cbl-b, such as a recombinant Cbl-b protein or truncated variants thereof, which generally preserve the binding ability of naturally occurring Cbl-b or naturally occurring variants of Cbl-b (e.g., the ability to bind to an E2 enzyme).

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions may be sterile.

“Excipients” as used herein include pharmaceutically acceptable excipients, carriers, vehicles or stabilizers that are nontoxic to the cell or mammal being exposed thereto at the dosages and concentrations employed. Often the physiologically acceptable excipient is an aqueous pH buffered solution.

Reference to a compound as described in a pharmaceutical composition, or to a compound as described in a claim to a pharmaceutical composition, refers to the compound described by the formula recited in the pharmaceutical composition, without the other elements of the pharmaceutical composition, that is, without carriers, excipients, etc.

The terms “treating” or “treatment” of a disease refer to executing a protocol, which may include administering one or more therapeutic agent to an individual (human or otherwise), in an effort to obtain beneficial or desired results in the individual, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total). “Treatment” also can mean prolonging survival as compared to expected survival of an individual not receiving treatment. Further, “treating” and “treatment” may occur by administration of one dose of a therapeutic agent or therapeutic agents, or may occur upon administration of a series of doses of a therapeutic agent or therapeutic agents. “Treating” or “treatment” does not require complete alleviation of signs or symptoms, and does not require a cure. “Treatment” also can refer to clinical intervention, such as administering one or more therapeutic agents to an individual, designed to alter the natural course of the individual or cell being treated (i.e., to alter the course of the individual or cell that would occur in the absence of the clinical intervention). The term “therapeutic agent” can refer to a Cbl-b inhibitor, a modified immune cell or compositions thereof.

As used herein, an “individual” or a “subject” is a mammal. A “mammal” for purposes of treatment includes humans; non-human primates; domestic and farm animals; and zoo, sports, or pet animals, such as dogs, horses, rabbits, cattle, pigs, hamsters, gerbils, mice, ferrets, rats, cats, etc. In some embodiments, the individual or subject is human.

As used herein, the term “T-cell dysfunction” refers to a state of reduced immune responsiveness to antigenic stimulation. The term “T-cell dysfunction” includes common elements of both T-cell exhaustion and/or T-cell anergy in which antigen recognition may occur, but the ensuing immune response is ineffective to control tumor growth. The term “T-cell dysfunction” also includes being refractory or unresponsive to antigen recognition, such as, impaired capacity to translate antigen recognition to downstream T-cell effector functions, such as proliferation, cytokine production and/or target cell killing.

The term “T-cell anergy” refers to the state of unresponsiveness to antigen stimulation resulting from incomplete or insufficient signals delivered through the T-cell receptor. “T-cell anergy” can also result upon stimulation with antigen in the absence of co-stimulation, resulting in the cell becoming refractory to subsequent activation by the antigen even in the context of co-stimulation.

The term “T-cell exhaustion” refers to a state of T-cell dysfunction that arises from sustained TCR signaling that can occur during cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cell.

A “T-cell dysfunction disorder” is a disorder or condition characterized by decreased responsiveness of T-cells to antigenic stimulation. Decreased responsiveness may result in ineffective control of a tumor. In some embodiments, the term “T-cell dysfunction disorder” encompasses cancer such as a hematologic cancer or a non-hematologic cancer. In some embodiments, a “T-cell dysfunctional disorder” is one in which T-cells are anergic or have decreased ability to secrete cytokines, proliferate or execute cytolytic activity.

“Enhancing T-cell function” means to induce, cause or stimulate a T-cell to have a sustained or amplified biological function, or renew or reactivate exhausted or inactive T-cells. Examples of enhanced T-cell function include increased T-cell activation (e.g., increased cytokine production, increased expression of T-cell activation markers, etc.), increased T-cell proliferation, decreased T-cell exhaustion, and/or decreased T-cell tolerance relative to the state of the T-cells before treatment with a Cbl-b inhibitor. Methods of measuring enhancement of T-cell function are known in the art.

“Proliferation” is used herein to refer to the proliferation of a cell. Increased proliferation encompasses the production of a greater number of cells relative to a baseline value. Decreased proliferation encompasses the production of a reduced number of cells relative to a baseline value. In some embodiments, the cell is an immune cell such as a T-cell and increased proliferation is desired. In some embodiments, the cell is a cancer cell and reduced proliferation is desired.

“Alkyl” as used herein refers to a saturated linear (i.e., unbranched) or branched univalent hydrocarbon chain or combination thereof. Particular alkyl groups are those having a designated number of carbon atoms, for example, an alkyl group having 1 to 20 carbon atoms (a “C₁-C₂₀ alkyl”), having 1 to 10 carbon atoms (a “C₁-C₁₀” alkyl), having 1 to 8 carbon atoms (a “C₁-C₈ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear (i.e., unbranched) or branched univalent hydrocarbon chain or combination thereof, having at least one site of olefinic unsaturation (i.e., having at least one moiety of the formula C═C). Particular alkenyl groups are those having a designated number of carbon atoms, for example, an alkenyl group having 2 to 20 carbon atoms (a “C₂-C₂₀ alkenyl”), having 2 to 10 carbon atoms (a “C₂-C₁₀” alkenyl), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). The alkenyl group may be in “cis” or “trans” configurations or, alternatively, in “E” or “Z” configurations. Examples of alkenyl groups include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs, and isomers thereof, and the like.

“Alkynyl” as used herein refers to an unsaturated linear (i.e., unbranched) or branched univalent hydrocarbon chain or combination thereof, having at least one site of acetylenic unsaturation (i.e., having at least one moiety of the formula C═C). Particular alkynyl groups are those having a designated number of carbon atoms, for example, an alkynyl group having 2 to 20 carbon atoms (a “C₂-C₂₀ alkynyl”), having 2 to 10 carbon atoms (a “C₂-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl groups include, but are not limited to, groups such as ethynyl (or acetylenyl), prop-1-ynyl, prop-2-ynyl (or propargyl), but-1-ynyl, but-2-ynyl, but-3-ynyl, homologs, and isomers thereof, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₄-C₅ alkylene”), 1 to 4 carbon atoms (a “C₁-C₄ alkylene”), or 1 to 3 carbon atoms (a “C₄-C₃ alkylene”). Examples of alkylene groups include, but are not limited to, groups such as methylene (—CH₂—), ethylene (—CH₂CH₂—), propylene (—CH₂CH₂CH₂—), butylene (—CH₂CH₂CH₂CH₂—), and the like.

“Cycloalkyl” as used herein refers to non-aromatic, saturated or unsaturated, cyclic univalent hydrocarbon structures. Particular cycloalkyl groups are those having a designated number of annular (i.e., ring) carbon atoms, for example, a cycloalkyl group having from 3 to 12 annular carbon atoms (a “C₃-C₁₂ cycloalkyl”). A particular cycloalkyl is a cyclic hydrocarbon having from 3 to 8 annular carbon atoms (a “C₃-C₈ cycloalkyl”), or having 3 to 6 annular carbon atoms (a “C₃-C₆ cycloalkyl”). Cycloalkyl can consist of one ring, such as cyclohexyl, or multiple rings, such as adamantyl, but excludes aryl groups. A cycloalkyl comprising more than one ring may be fused, spiro, or bridged, or combinations thereof. Examples of cycloalkyl groups include, but are not limited to, cyclopropyl, cyclobutyl, cyclopentyl, cyclohexyl, 1-cyclohexenyl, 3-cyclohexenyl, cycloheptyl, norbornyl, and the like.

“Cycloalkylene” as used herein refers to the same residues as cycloalkyl, but having bivalency. Particular cycloalkylene groups are those having 3 to 12 annular carbon atoms (a “C₃-C₁₂ cycloalkylene”), having from 3 to 8 annular carbon atoms (a “C₃-C₈ cycloalkylene”), or having 3 to 6 annular carbon atoms (a “C₃-C₆ cycloalkylene”). Examples of cycloalkylene groups include, but are not limited to, cyclopropylene, cyclobutylene, cyclopentylene, cyclohexylene, 1,2-cyclohexenylene, 1,3-cyclohexenylene, 1,4-cyclohexenylene, cycloheptylene, norbornylene, and the like.

“Aryl” as used herein refers to an aromatic carbocyclic group having a single ring (e.g., phenyl), or multiple condensed rings (e.g., naphthyl or anthryl) where one or more of the condensed rings may not be aromatic. Particular aryl groups are those having from 6 to 14 annular (i.e., ring) carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position. Examples of aryls include, but are not limited to, groups such as phenyl, naphthyl, 1-naphthyl, 2-naphthyl, 1,2,3,4-tetrahydronaphthalen-6-yl, and the like.

“Carbocyclyl” or “carbocyclic” refers to a univalent cyclic group in which all of the ring members are carbon atoms, such as cyclohexyl, phenyl, 1,2-dihydronaphthyl, etc.

“Arylene” as used herein refers to the same residues as aryl, but having bivalency. Particular arylene groups are those having from 6 to 14 annular carbon atoms (a “C₆-C₁₄ arylene”). Examples of arylene include, but are not limited to, groups such as phenylene, o-phenylene (i.e., 1,2-phenylene), m-phenylene (i.e., 1,3-phenylene), p-phenylene (i.e., 1,4-phenylene), naphthylene, 1,2-naphthylene, 1,3-naphthylene, 1,4-naphthylene, 2,7-naphthylene, 2,6-naphthylene, and the like.

“Heteroaryl” as used herein refers to an unsaturated aromatic cyclic group having from 1 to 14 annular carbon atoms and at least one annular heteroatom, including, but not limited to, heteroatoms such as nitrogen, oxygen, and sulfur. A heteroaryl group may have a single ring (e.g., pyridyl or imidazolyl) or multiple condensed rings (e.g., indolizinyl, indolyl, or quinolinyl) where at least one of the condensed rings is aromatic. Particular heteroaryl groups are 5- to 14-membered rings having 1 to 12 annular carbon atoms and 1 to 6 annular heteroatoms independently selected from the group consisting of nitrogen, oxygen, and sulfur (a “5- to 14-membered heteroaryl”); 5- to 10-membered rings having 1 to 8 annular carbon atoms and 1 to 4 annular heteroatoms independently selected from the group consisting of nitrogen, oxygen, and sulfur (a “5- to 10-membered heteroaryl”); or 5-, 6-, or 7-membered rings having 1 to 5 annular carbon atoms and 1 to 4 annular heteroatoms independently selected from the group consisting of nitrogen, oxygen, and sulfur (a “5- to 7-membered heteroaryl”). In one variation, heteroaryl includes monocyclic aromatic 5-, 6-, or 7-membered rings having from 1 to 6 annular carbon atoms and 1 to 4 annular heteroatoms independently selected from the group consisting of nitrogen, oxygen, and sulfur. In another variation, heteroaryl includes polycyclic aromatic rings having from 1 to 12 annular carbon atoms and 1 to 6 annular heteroatoms independently selected from the group consisting of nitrogen, oxygen, and sulfur. A heteroaryl group having more than one ring where at least one ring is non-aromatic may be connected to the parent structure at either an aromatic ring position or at a non-aromatic ring position. Examples of heteroaryl include, but are not limited to, groups such as pyridyl, benzimidazolyl, benzotriazolyl, benzo[b]thienyl, quinolinyl, indolyl, benzothiazolyl, and the like. “Heteroaryl” also includes moieties such as

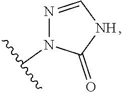 [SMILES: *n1nc[nH]c1=O]

2,4-dihydro-3H-1,2,4-triazol-3-one-2-yl, which has the tautomeric structure

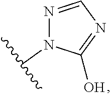 [SMILES: *n1ncnc1O]

1H-1,2,4-triazol-5-ol-1-yl.

“Heterocyclyl” and “heterocyclic groups” as used herein refer to non-aromatic saturated or partially unsaturated cyclic groups having the number of atoms and heteroatoms as specified, or if no number of atoms or heteroatoms is specified, having at least three annular atoms, from 1 to 14 annular carbon atoms, and at least one annular heteroatom, including, but not limited to, heteroatoms such as nitrogen, oxygen, and sulfur. A heterocyclic group may have a single ring (e.g., tetrahydrothiophenyl, oxazolidinyl) or multiple condensed rings (e.g., decahydroquinolinyl, octahydrobenzo[d]oxazolyl). Multiple condensed rings include, but are not limited to, bicyclic, tricyclic, and quadracylic rings, as well as bridged or spirocyclic ring systems. Examples of heterocyclic groups include, but are not limited to, aziridinyl, azetidinyl, pyrrolidinyl, piperidinyl, oxiranyl, oxetanyl, tetrahydrofuranyl, tetrahydropyranyl, oxazolidinyl, piperazinyl, morpholinyl, dioxanyl, 3,6-dihydro-2H-pyranyl, 2,3-dihydro-1H-imidazolyl, and the like.

“Heteroarylene” as used herein refers to the same residues as heteroaryl, but having bivalency. Particular heteroarylene groups are 5- to 14-membered rings having 1 to 12 annular carbon atoms and 1 to 6 annular heteroatoms independently selected from the group consisting of nitrogen, oxygen, and sulfur (a “5- to 14-membered heteroarylene”); 5- to 10-membered rings having 1 to 8 annular carbon atoms and 1 to 4 annular heteroatoms independently selected from the group consisting of nitrogen, oxygen, and sulfur (a “5- to 10-membered heteroarylene”); or 5-, 6-, or 7-membered rings having 1 to 5 annular carbon atoms and 1 to 4 annular heteroatoms independently selected from the group consisting of nitrogen, oxygen, and sulfur (a “5- to 7-membered heteroarylene”). Examples of heteroarylene include, but are not limited to, groups such as pyridylene, benzimidazolylene, benzotriazolylene, benzo[b]thienylene, quinolinylene, indolylene, benzothiazolylene, and the like.

“Halo” or “halogen” refers to elements of the Group 17 series having atomic number 9 to 85. Halo groups include fluoro, chloro, bromo, and iodo.

“Haloalkyl,” “haloalkylene,” “haloaryl,” “haloarylene,” “haloheteroaryl,” and similar terms refer to a moiety substituted with at least one halo group. Where a haloalkyl moiety or other halo-substituted moiety is substituted with more than one halogen, it may be referred to by using a prefix corresponding to the number of halogen moieties attached. For example, dihaloaryl, dihaloalkyl, trihaloaryl, trihaloalkyl, etc., refer to aryl and alkyl substituted with two (“di”) or three (“tri”) halo groups, which may be, but are not necessarily, the same halo; thus, for example, the haloaryl group 4-chloro-3-fluorophenyl is within the scope of dihaloaryl. The subset of haloalkyl groups in which each hydrogen of an alkyl group is replaced with a halo group is referred to as a “perhaloalkyl.” A particular perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃). “Haloalkyl” includes monohaloalkyl, dihaloalkyl, trihaloalkyl, perhaloalkyl, and any other number of halo substituents possible on an alkyl group; and similarly for other groups such as haloalkylene, haloaryl, haloarylene, haloheteroaryl, etc.

“Amino” refers to the group —NH₂.

“Oxo” refers to the group ═O, that is, an oxygen atom doubly bonded to carbon or another element.

“Optionally substituted,” unless otherwise specified, means that a group is unsubstituted or substituted by one or more (e.g., 1, 2, 3, 4, or 5) of the substituents listed for that group, in which the substituents may be the same or different. In one embodiment, an optionally substituted group is unsubstituted. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, or 1 to 5 substituents. When multiple substituents are present, each substituent is independently chosen unless indicated otherwise. For example, each (C₁-C₄ alkyl) substituent on the group —N(C₁-C₄ alkyl)(C₁-C₄ alkyl) can be selected independently from the other, so as to generate groups such as —N(CH₃)(CH₂CH₃), etc.

In addition to the disclosure herein, the term “substituted,” when used to modify a specified group or radical, can also mean that one or more hydrogen atoms of the specified group or radical are each, independently of one another, replaced with the same or different substituent groups as defined herein. In some embodiments, a group that is substituted has 1, 2, 3, or 4 substituents, 1, 2, or 3 substituents, 1 or 2 substituents, or 1 substituent.

Substituents can be attached to any chemically possible location on the specified group or radical, unless indicated otherwise. Thus, —C₁-C₈ alkyl-OH includes, for example, —CH₂CH₂OH and —CH(OH)—CH₃, and —CH₂C(OH)(CH₃)₂.

Unless a specific isotope of an element is indicated in a formula, the disclosure includes all isotopologues of the compounds disclosed herein, such as, for example, deuterated derivatives of the compounds (where H can be 2H, i.e., D). Isotopologues can have isotopic replacements at any or at all locations in a structure, or can have atoms present in natural abundance at any or all locations in a structure.

The disclosure also includes any or all of the stereochemical forms, including any enantiomeric or diastereomeric forms of the compounds described herein, and cis/trans or E/Z isomers. Unless stereochemistry is explicitly indicated in a chemical structure or name, the structure or name is intended to embrace all possible stereoisomers of a compound depicted. In addition, where a specific stereochemical form is depicted, it is understood that all other stereochemical forms are also described and embraced by the disclosure, as well as the general non-stereospecific form and mixtures of the disclosed compounds in any ratio, including mixtures of two or more stereochemical forms of a disclosed in any ratio, such that racemic, non-racemic, enantioenriched and scalemic mixtures of a compound are embraced. Compositions comprising a disclosed compound also are intended, such as a composition of substantially pure compound, including a specific stereochemical form thereof. Compositions comprising a mixture of disclosed compounds in any ratio also are embraced by the disclosure, including compositions comprising mixtures of two or more stereochemical forms of a disclosed compound in any ratio, such that racemic, non-racemic, enantioenriched and scalemic mixtures of a compound are embraced by the disclosure. If stereochemistry is explicitly indicated for one portion or portions of a molecule, but not for another portion or portions of a molecule, the structure is intended to embrace all possible stereoisomers for the portion or portions where stereochemistry is not explicitly indicated.

The disclosure also embraces any and all tautomeric forms of the compounds described herein.

The disclosure is intended to embrace all salts of the compounds described herein, as well as methods of using such salts of the compounds. In one embodiment, the salts of the compounds comprise pharmaceutically acceptable salts. Pharmaceutically acceptable salts are those salts that can be administered as drugs or pharmaceuticals to humans and/or animals and that, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, also can be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, also can be prepared. For lists of pharmaceutically acceptable salts, see, for example, P. H. Stahl and C. G. Wermuth (eds.) “Handbook of Pharmaceutical Salts, Properties, Selection and Use” Wiley-VCH, 2011 (ISBN: 978-3-90639-051-2). Several pharmaceutically acceptable salts are also disclosed in Berge, J. Pharm. Sci. 66:1 (1977).

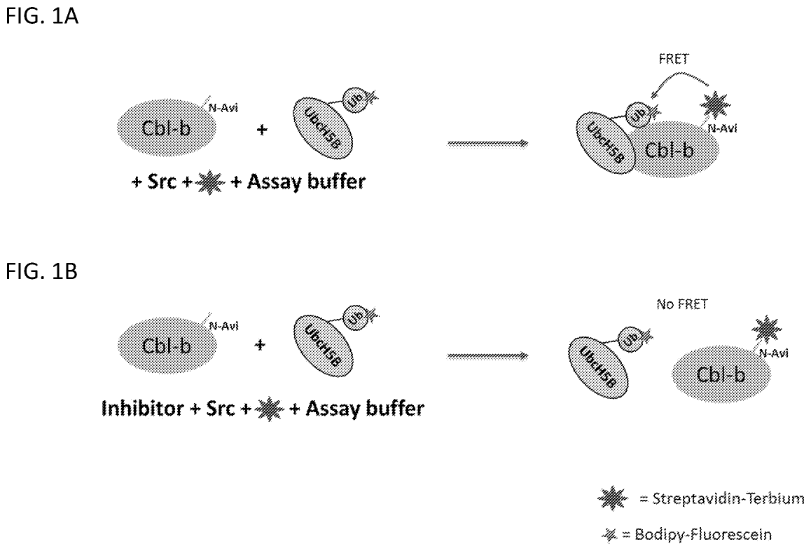

As described in Biological Example 1, FIG. 1A, and FIG. 1B, the Cbl-b activity assay (Cbl-b inhibition assay) used to measure the IC₅₀ values for Cbl-b inhibition uses a mixture comprising an N-terminal biotinylated Avi-tagged Cbl-b, an E2 enzyme (i.e., UbcH5B) irreversibly conjugated to Bodipy-Fluorescein tagged ubiquitin (i.e., UbcH5B-Ub), a Src kinase, streptavidin-terbium, and assay buffer. FIG. 1A shows the assay in the absence of a Cbl-b inhibitor, where phosphorylation of Cbl-b by the Src kinase allows UbcH5B-Ub to bind to Cbl-b and brings the Bodipy-Fluorescein tagged ubiquitin in proximity to the streptavidin-terbium bound to the N-terminal biotinylated Avi-tagged Cbl-b. The resulting complex produces a FRET signal indicating that Cbl-b is active or not subject to inhibition. FIG. 1B shows the assay in the presence of a Cbl-b inhibitor, where Cbl-b is unable to bind UbcH5B-Ub. The lack of a FRET signal indicates Cbl-b is inhibited by the Cbl-b inhibitor. In FIG. 1A and FIG. 1B, Ub indicates ubiquitin; N-Avi indicates biotinylated Avi-tag at the N-terminus of Cbl-b; and inhibitor indicates Cbl-b inhibitor. In one embodiment, the Cbl-b activity assay (Cbl-b inhibition assay) used to measure IC₅₀ for inhibition of Cbl-b uses the conditions described in Biological Example 1, with 100 mM NaCl and 60 nM Src kinase. In another embodiment, an alternate assay, the “low-salt/low-Src kinase assay,” uses the conditions described in Biological Example 1, but with 50 mM NaCl and 30 nM Src kinase.

The Cbl-b binding assay is also described in Biological Example 1, and provides the dissociation constant K_{D} for the binding of the compounds disclosed herein.

It is appreciated that certain features disclosed herein, which are, for clarity, described in the context of separate embodiments, also may be provided in combination in a single embodiment. Conversely, various features disclosed herein, which are, for brevity, described in the context of a single embodiment, also may be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables also are specifically embraced by the present disclosure and are disclosed herein just as if each and every such subcombination of chemical groups was individually and explicitly disclosed herein.

It is understood that aspects and embodiments described herein as “comprising” include “consisting of” and “consisting essentially of” embodiments.

As used herein and in the appended claims, the singular forms “a,”,“an,” and “the” include plural referents unless otherwise indicated or clear from context.

Reference to “about” a value or parameter herein includes (and describes) embodiments that are directed to that value or parameter per se. For example, description referring to “about X” includes description of “X.”

When numerical ranges of compounds are given, all compounds within those numerical limits designated “a” and “b” are included, unless expressly excluded. For example, reference to compounds 638-640 refers to compounds 638, 639a, 639b, and 640.

### II. Compounds

The compounds disclosed herein are of the general form:

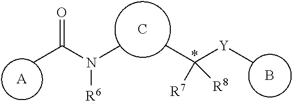

where

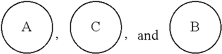

indicate ring systems (Ring A, Ring C, Ring B).

The modular nature of the compounds allows synthesis to be performed in a stepwise manner, as outlined herein. The compounds disclosed herein can be prepared by a variety of methods, as described in general schemes below, and as described for specific compounds in the Examples.

For compounds and pharmaceutical compositions, and for methods using compounds and pharmaceutical compositions, the carbon atom highlighted by the asterisk in the structure below (to which Ring C, R⁷, R⁸, and Y are attached):

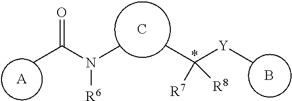

will be asymmetric when R⁷ and R⁸ are different substituents, and two different absolute configurations will be available at that carbon atom. When Y is C(R⁹)(R¹⁰), the absolute stereochemical configuration at the carbon atom highlighted by the asterisk with a lower IC₅₀ value for Cbl-b inhibition is typically (R) using the Cahn-Ingold-Prelog rules. When Y is S (that is, when Y is sulfur), the absolute stereochemical configuration at the carbon atom highlighted by the asterisk with a lower IC₅₀ value for Cbl-b inhibition is typically (S). These configurations are embraced by the disclosure; that is, when Y is C(R⁹)(R¹⁰), the stereochemical configuration at the carbon atom highlighted by the asterisk can be (R), and when Y is S (sulfur), the stereochemical configuration at the carbon atom highlighted by the asterisk can be (S). However, the opposite configurations also are embraced by the disclosure; that is, when Y is C(R⁹)(R¹⁰), the stereochemical configuration at the carbon atom highlighted by the asterisk can be (S), and when Y is S (sulfur), the stereochemical configuration at the carbon atom highlighted by the asterisk can be (R). Mixtures of stereoisomers produced during synthesis, such as racemic mixtures of final compounds, can be separated into the respective enantiomers using common chromatography methods such as supercritical fluid chromatography in combination with chiral stationary phases, chiral column chromatography, or other methods known in the art.

Representative compounds of the disclosure are shown in Table 1.

| Compound | |
|---|---|
| number | Structure |
| 1 | 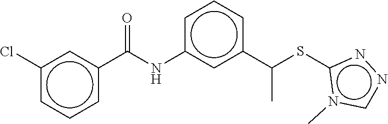 |
| 2 | 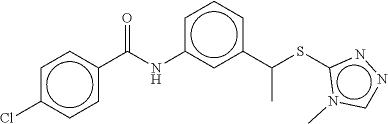 |
| 3 | 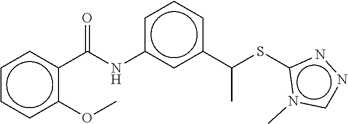 |
| 4 | 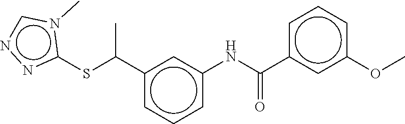 |
| 5 | 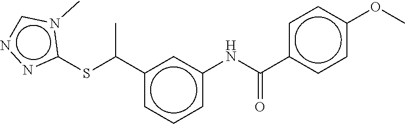 |
| 6 | 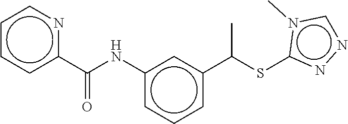 |
| 7 | 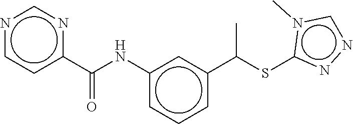 |
| 8 | 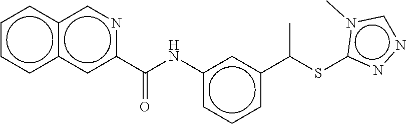 |
| 8a | 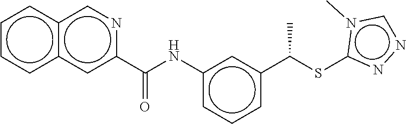 |
| 8b | 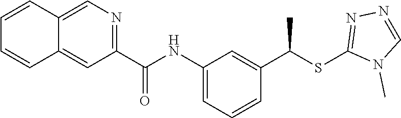 [SMILES: C[C@@H](Sc1nncn1C)c1cccc(NC(=O)c2cc3ccccc3cn2)c1] |
| 9 | 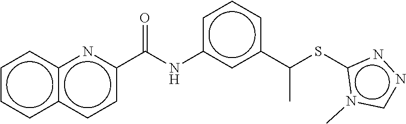 |
| 9a | 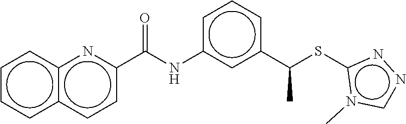 |
| 9b | 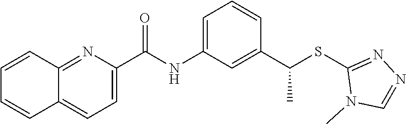 [SMILES: C[C@@H](Sc1nncn1C)c1cccc(NC(=O)c2ccc3ccccc3n2)c1] |
| 10 | 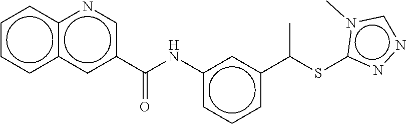 |
| 11 | 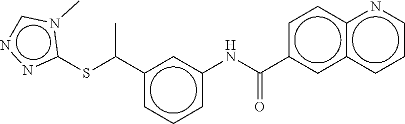 |
| 12 | 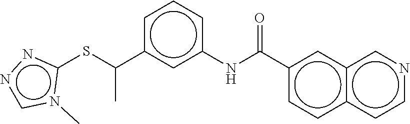 |
| 13 | 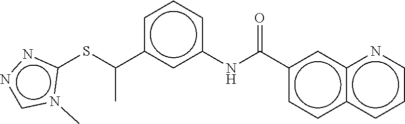 |
| 14 | 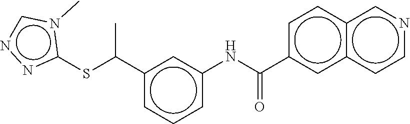 |
| 15 | 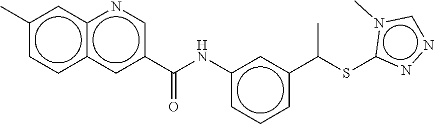 |
| 16 | 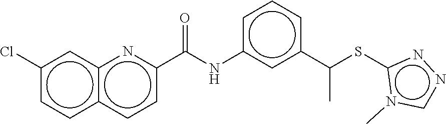 |
| 17 | 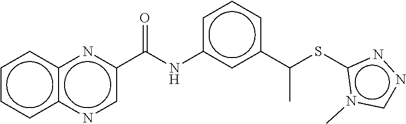 |
| 18 | 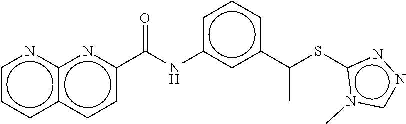 |
| 19 | 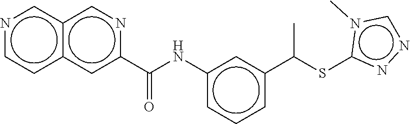 |
| 20 | 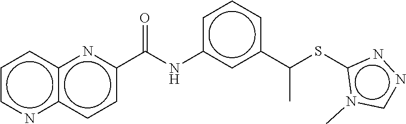 |
| 21 | 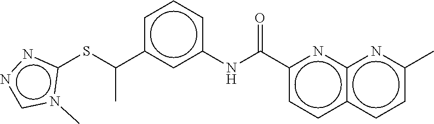 |
| 22 | 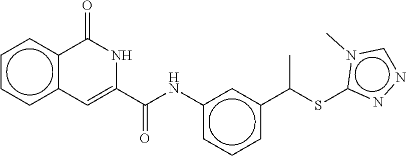 |
| 23 | 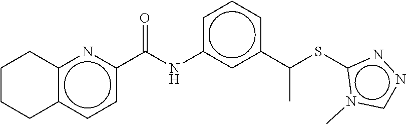 |
| 24 | 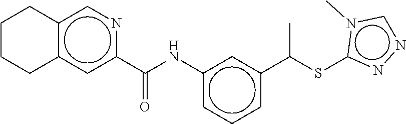 |
| 25 | 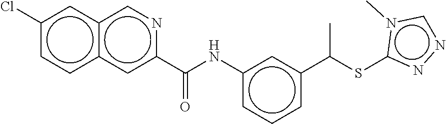 |
| 26 | 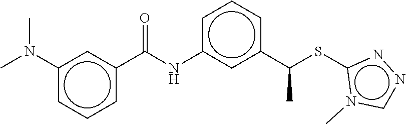 |
| 27 | 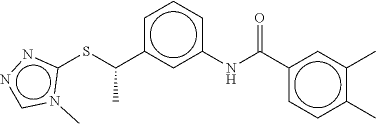 |
| 28 | 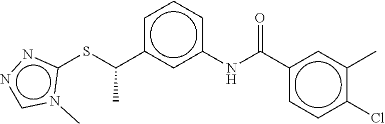 |
| 29 | 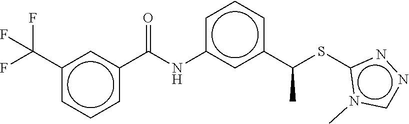 |
| 30 | 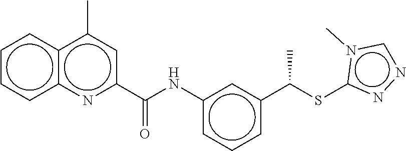 |
| 31 | 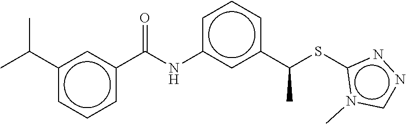 |
| 32 | 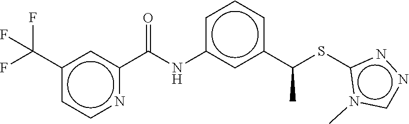 |
| 33 | 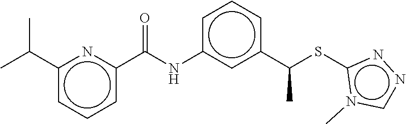 |
| 34 | 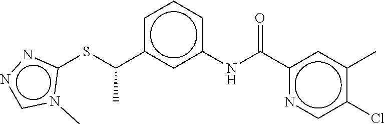 |
| 35 | 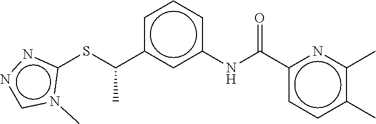 |
| 36 | 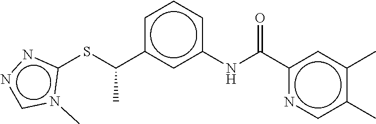 |
| 37 | 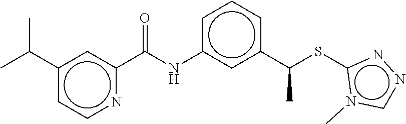 |
| 38 | 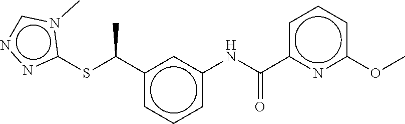 |
| 39 | 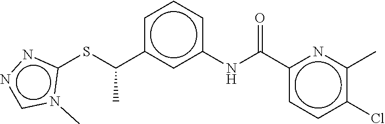 |
| 40 | 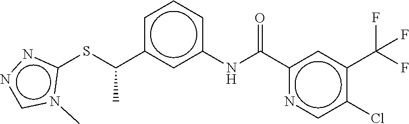 |
| 41 | 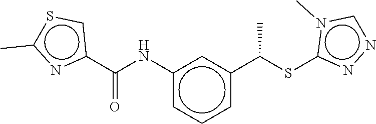 |
| 42 | 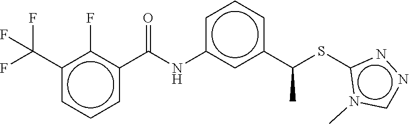 |
| 43 | 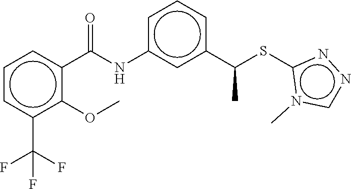 |
| 44 | 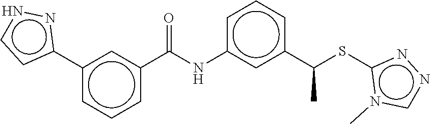 |
| 45 | 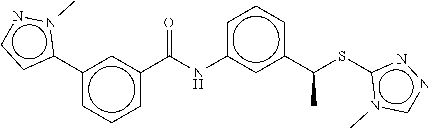 |
| 46 | 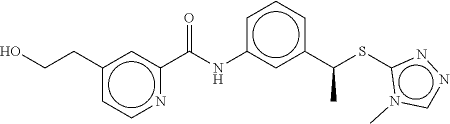 |
| 47 | 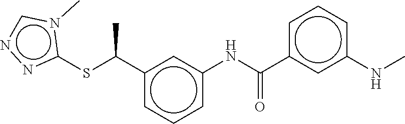 |
| 48 | 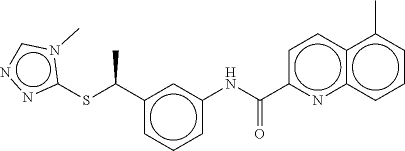 |
| 49 | 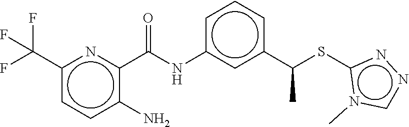 |
| 50 | 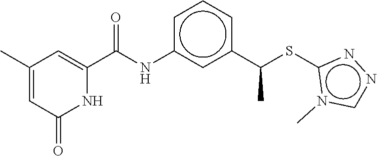 |
| 51 | 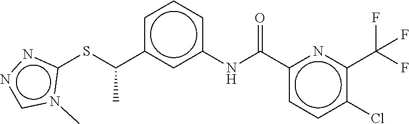 |
| 52 | 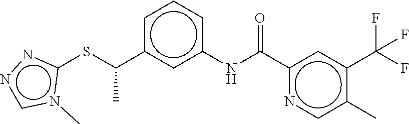 |
| 53 | 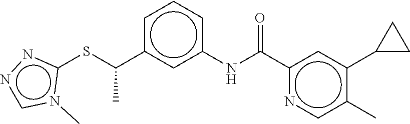 |
| 54 | 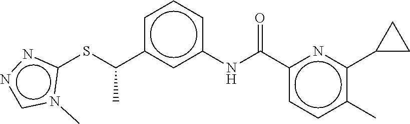 |
| 55 | 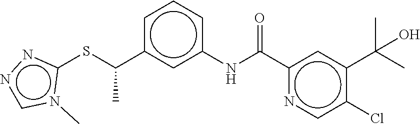 |
| 56 | 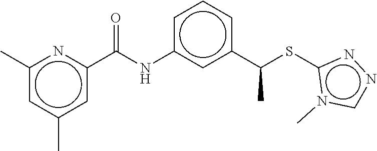 |
| 57a | 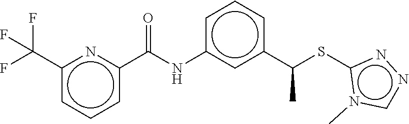 |
| 57b | 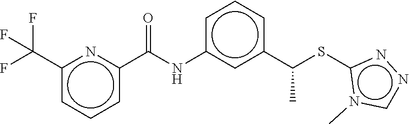 |
| 58 | 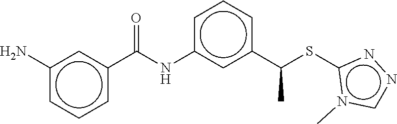 |
| 59 | 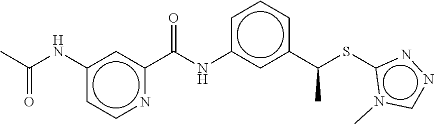 |
| 60 | 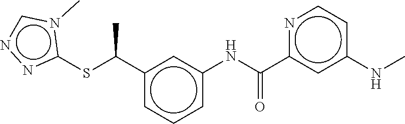 |
| 61 | 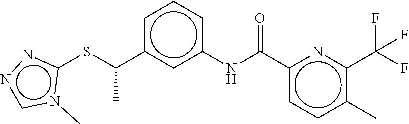 |
| 62 | 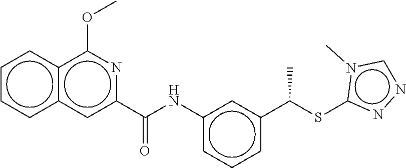 |
| 63 | 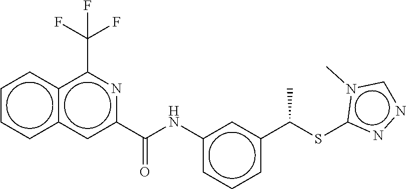 |
| 64 | 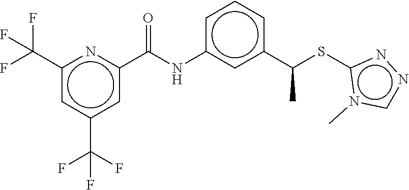 |
| 65 | 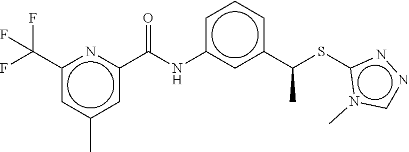 |
| 66 | 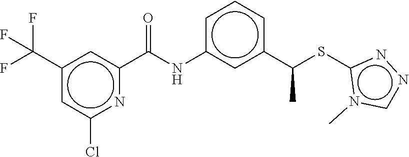 |
| 67 | 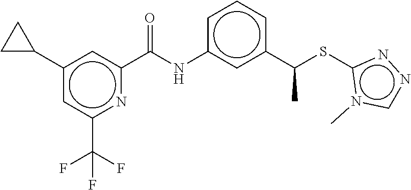 |
| 68 | 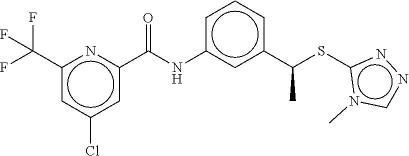 |
| 69 | 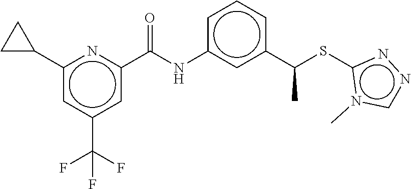 |
| 70 | 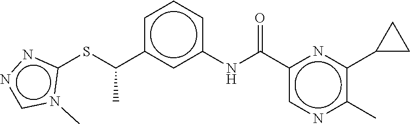 |
| 71 | 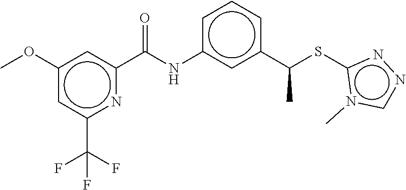 |
| 72 | 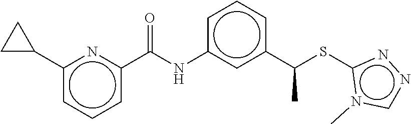 |
| 73 | 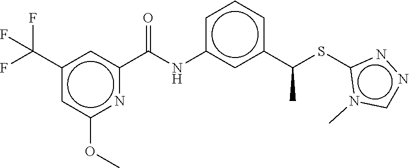 |
| 74 | 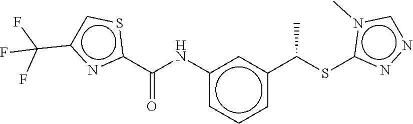 |
| 75 | 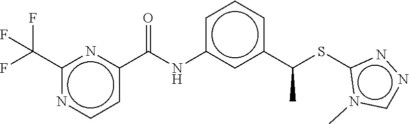 |
| 76 | 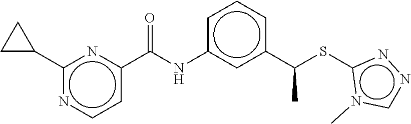 |
| 77 | 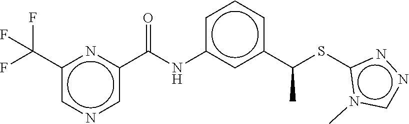 |
| 78 | 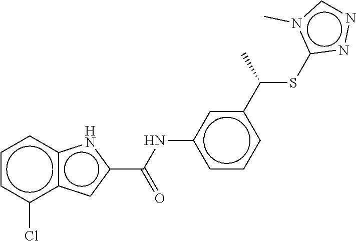 |
| 79 | 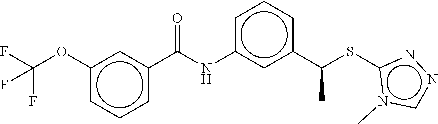 |
| 80 | 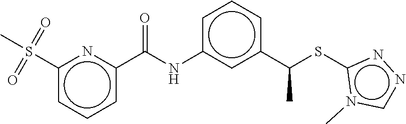 |
| 81 | 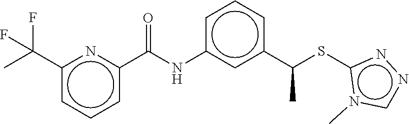 |
| 82 | 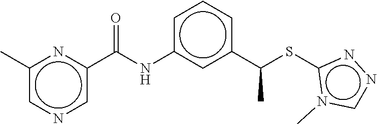 |
| 83 | 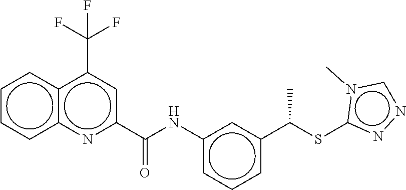 |
| 84 | 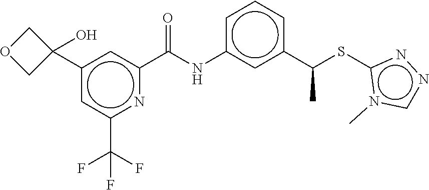 |
| 85 | 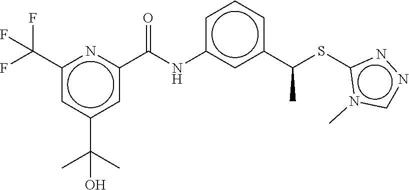 |
| 86 | 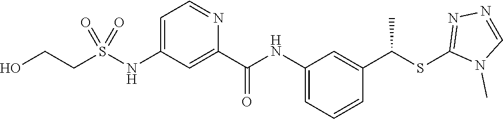 [SMILES: C[C@H](Sc1nncn1C)c1cccc(NC(=O)c2cc(NS(=O)(=O)CCO)ccn2)c1] |
| 87 | 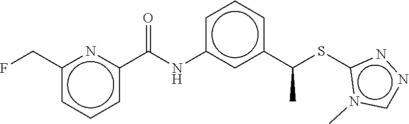 |
| 88 | 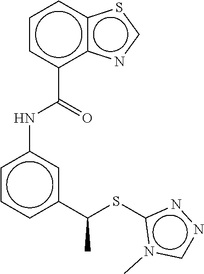 |
| 89 | 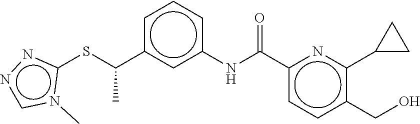 |
| 90 | 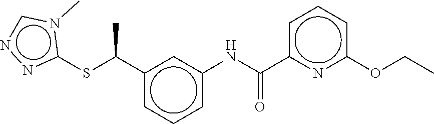 |
| 91 | 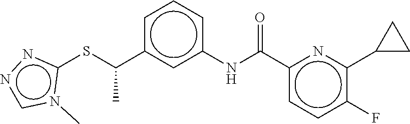 |
| 92 | 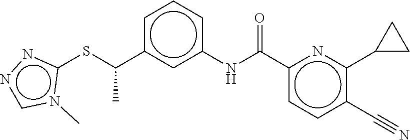 |
| 93 | 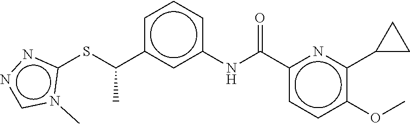 |
| 94 | 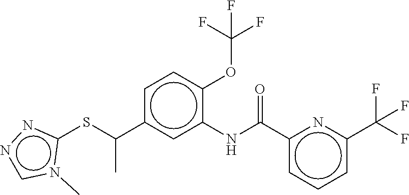 |
| 95 | 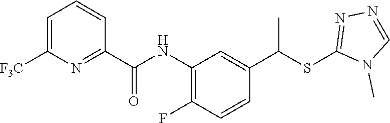 [SMILES: CC(Sc1nncn1C)c1ccc(F)c(NC(=O)c2cccc(C(F)(F)F)n2)c1] |
| 95a | 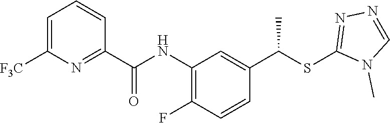 [SMILES: C[C@H](Sc1nncn1C)c1ccc(F)c(NC(=O)c2cccc(C(F)(F)F)n2)c1] |
| 95b | 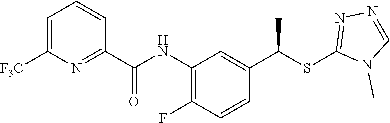 [SMILES: C[C@@H](Sc1nncn1C)c1ccc(F)c(NC(=O)c2cccc(C(F)(F)F)n2)c1] |
| 96 | 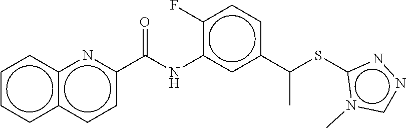 |
| 96a | 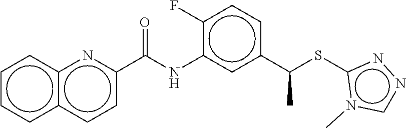 |
| 96b | 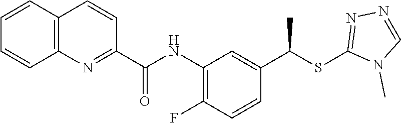 [SMILES: C[C@@H](Sc1nncn1C)c1ccc(F)c(NC(=O)c2ccc3ccccc3n2)c1] |
| 97 | 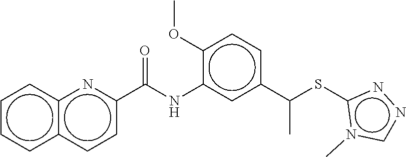 |
| 98 | 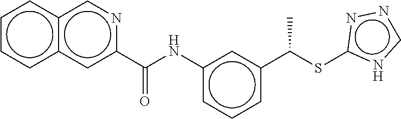 |
| 99 | 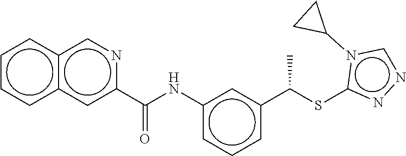 |
| 100 | 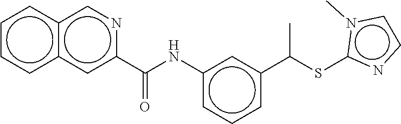 |
| 101 | 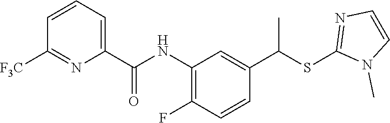 [SMILES: CC(Sc1nccn1C)c1ccc(F)c(NC(=O)c2cccc(C(F)(F)F)n2)c1] |
| 102a | 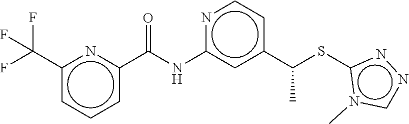 |
| 102b | 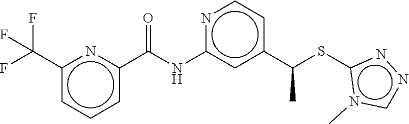 |
| 103 | 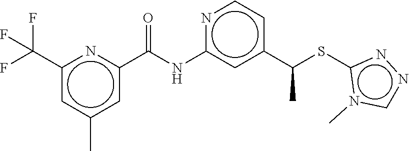 |
| 104 | 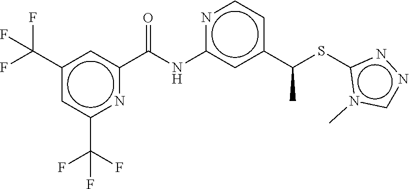 |
| 105 | 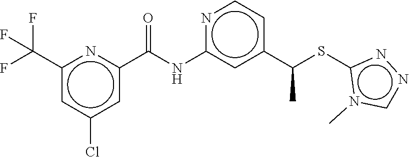 |
| 106 | 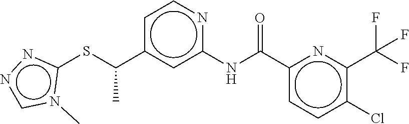 |
| 107 | 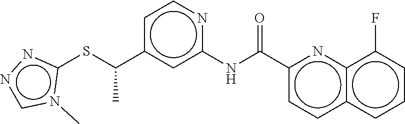 |
| 108 | 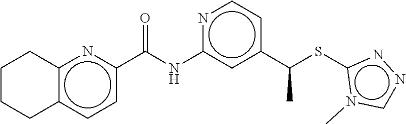 |
| 109 | 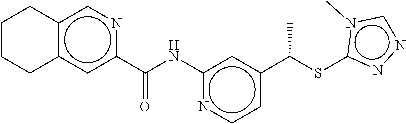 |
| 110 | 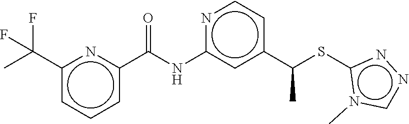 |
| 111 | 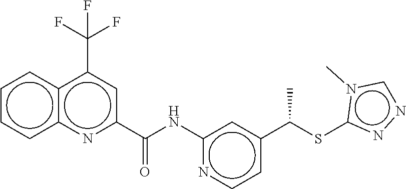 |
| 112 | 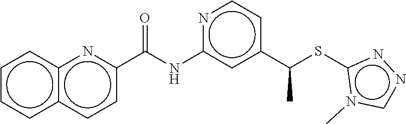 |
| 113 | 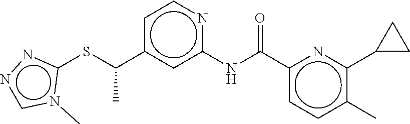 |
| 114 | 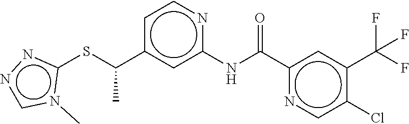 |
| 115 | 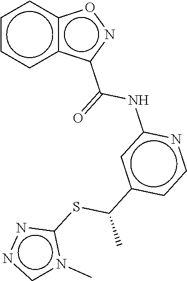 |
| 116 | 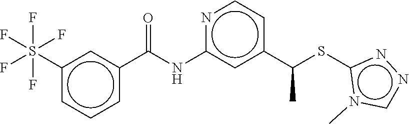 |
| 117 | 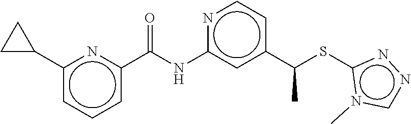 |
| 118 | 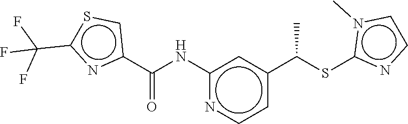 |
| 119 | 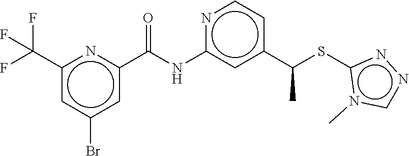 |
| 120 | 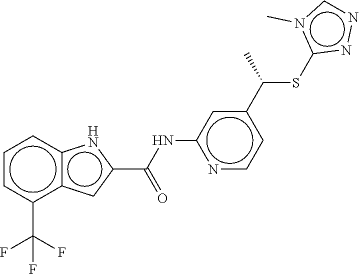 |
| 121 | 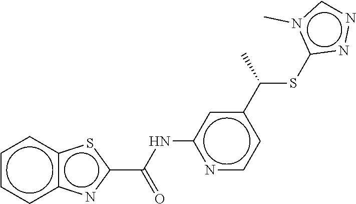 |
| 122 | 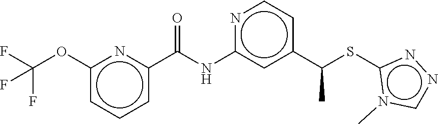 |
| 123 | 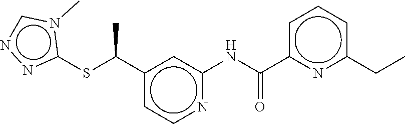 |
| 124 | 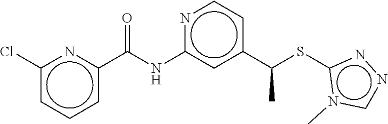 |
| 125 | 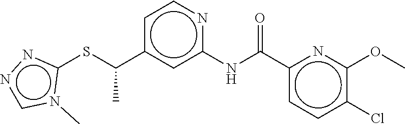 |
| 126 | 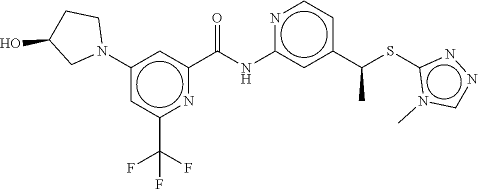 |
| 127 | 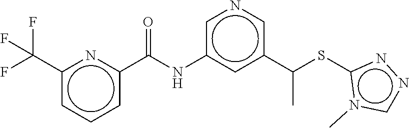 |
| 128a | 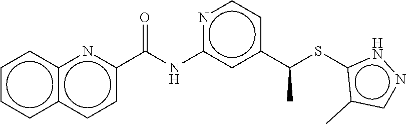 |
| 128b | 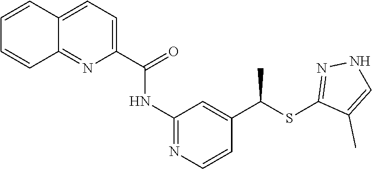 [SMILES: Cc1c[nH]nc1S[C@H](C)c1ccnc(NC(=O)c2ccc3ccccc3n2)c1] |
| 129 | 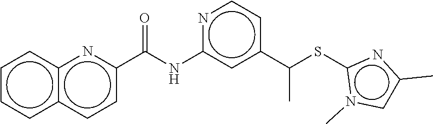 |
| 130a | 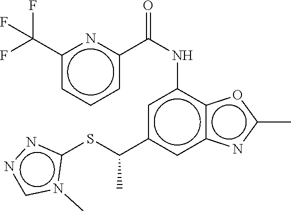 |
| 130b | 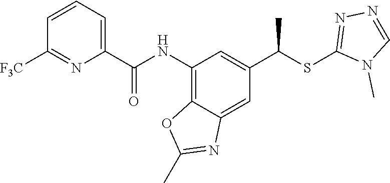 [SMILES: Cc1nc2cc([C@@H](C)Sc3nncn3C)cc(NC(=O)c3cccc(C(F)(F)F)n3)c2o1] |
| 131 | 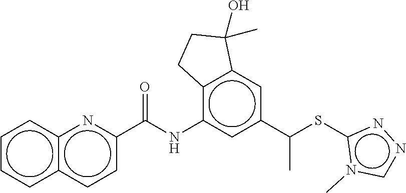 |
| 132 | 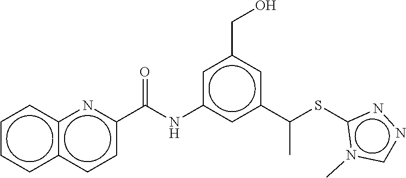 |
| 133 | 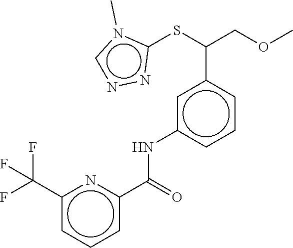 |
| 134 | 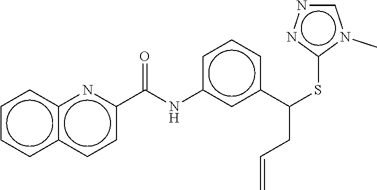 |
| 135 | 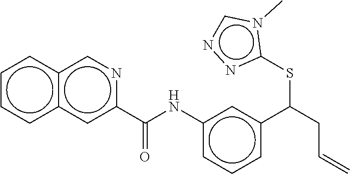 |
| 136 | 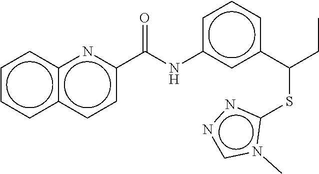 |
| 137 | 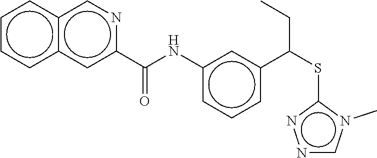 |
| 138 | 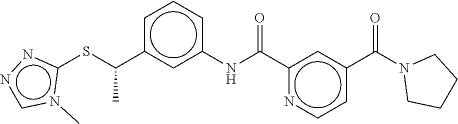 |
| 139 | 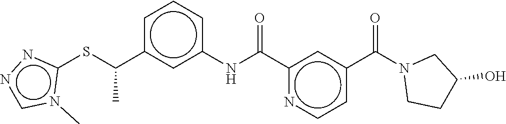 |
| 140 | 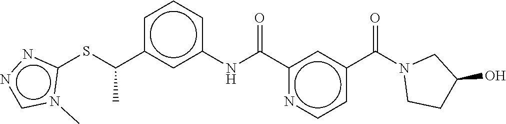 |
| 141 | 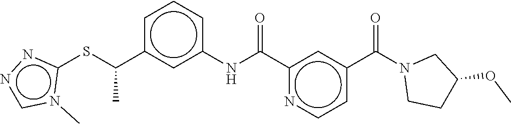 |
| 142 | 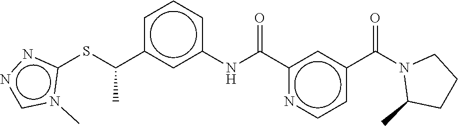 |
| 143 | 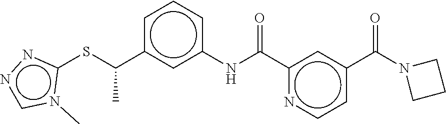 |
| 144 | 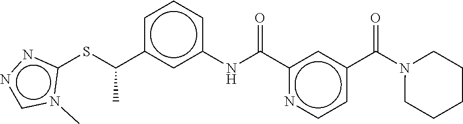 |
| 145 | 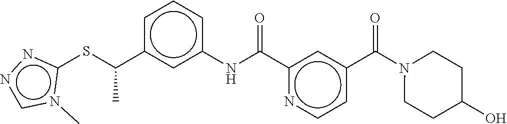 |
| 146 | 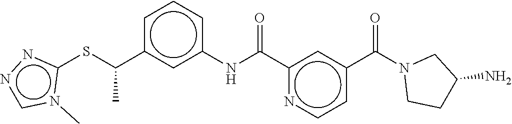 |
| 147 | 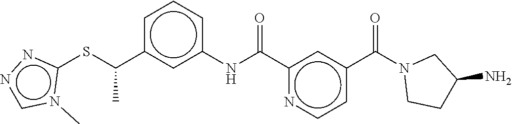 |
| 148 | 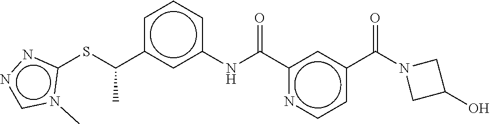 |
| 149 | 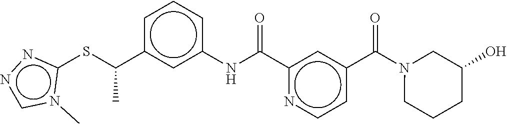 |
| 150 | 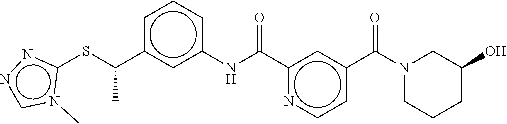 |
| 151 | 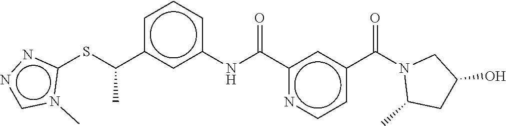 |
| 152 | 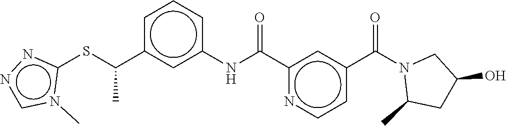 |
| 153 | 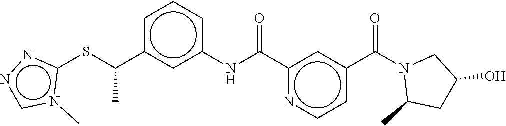 |
| 154 | 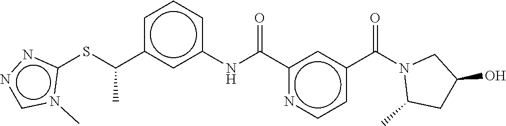 |
| 155 | 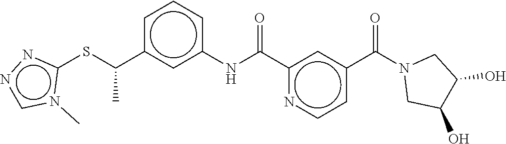 |
| 156 | 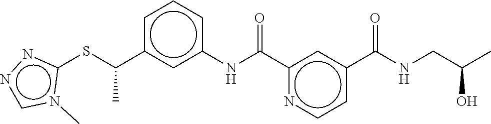 |
| 157 | 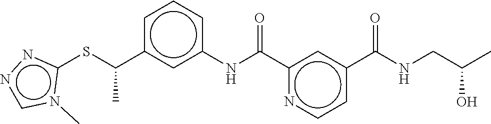 |
| 158 | 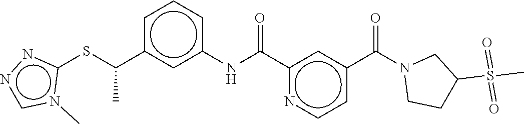 |
| 159 | 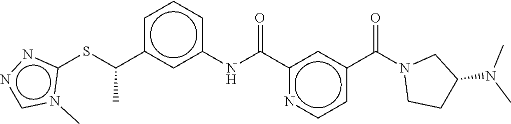 |
| 160 | 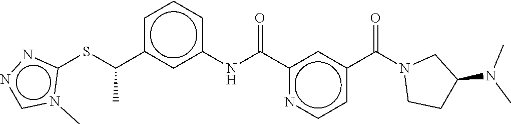 |
| 161 | 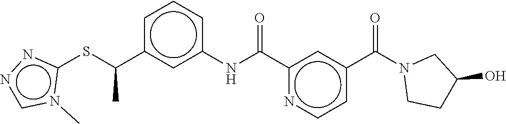 |
| 162 | 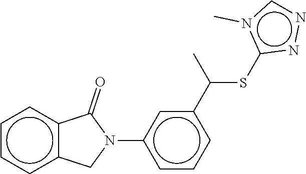 |
| 163 | 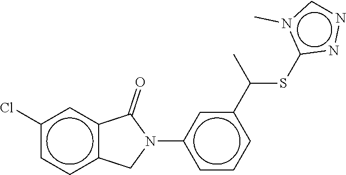 |
| 164 | 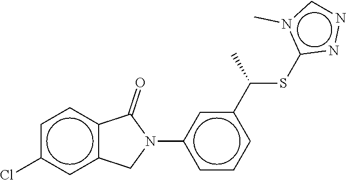 |
| 165 | 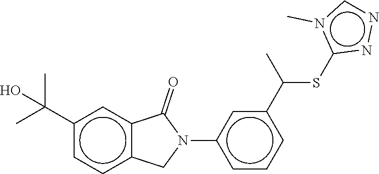 |
| 166 | 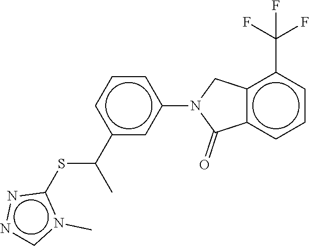 |
| 166a | 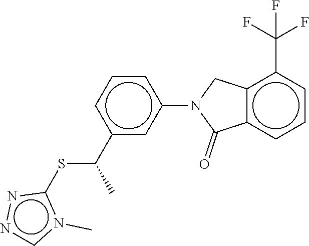 |
| 166b | 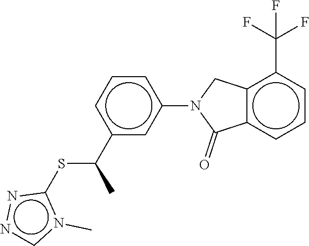 |
| 167 | 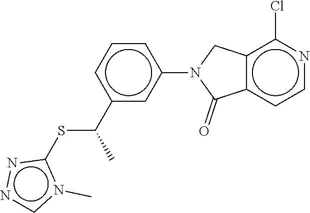 |
| 168 | 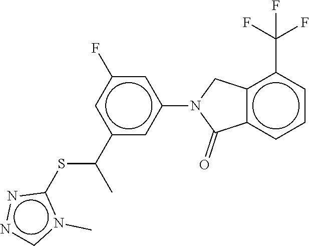 |
| 168a | 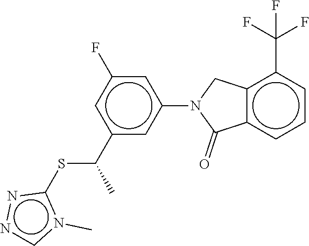 |
| 168b | 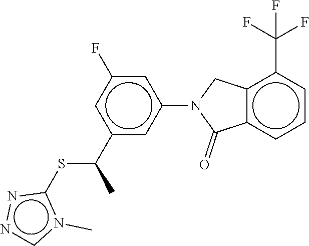 |
| 169 | 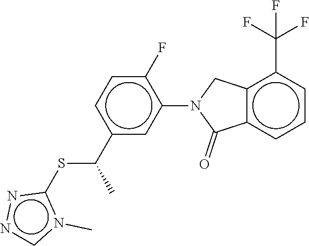 |
| 170 | 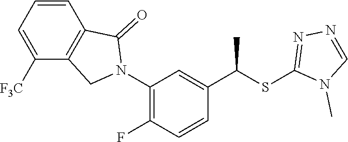 [SMILES: C[C@@H](Sc1nncn1C)c1ccc(F)c(N2Cc3c(cccc3C(F)(F)F)C2=O)c1] |
| 171 | 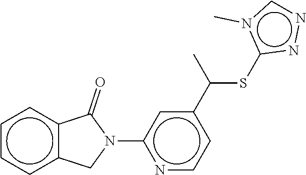 |
| 172a | 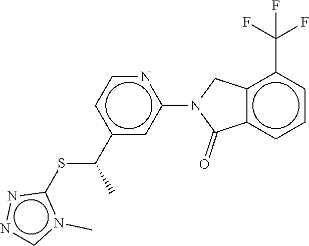 |
| 172b | 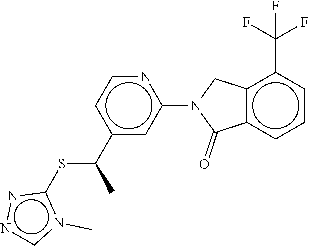 |
| 173 | 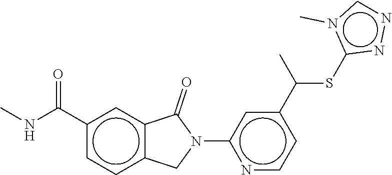 |
| 174 | 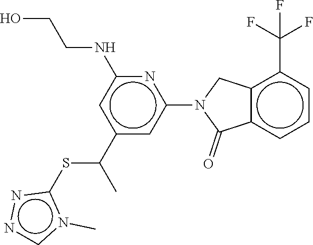 |
| 175 | 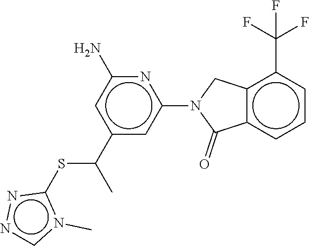 |
| 176 | 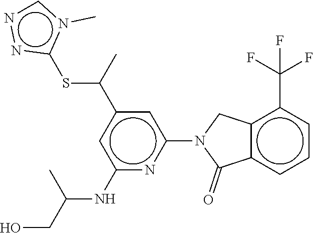 |
| 177 | 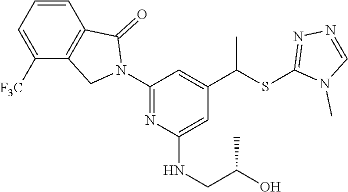 [SMILES: CC(Sc1nncn1C)c1cc(NC[C@H](C)O)nc(N2Cc3c(cccc3C(F)(F)F)C2=O)c1] |
| 178 | 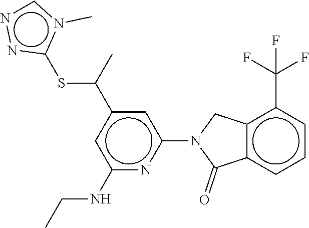 |
| 179 | 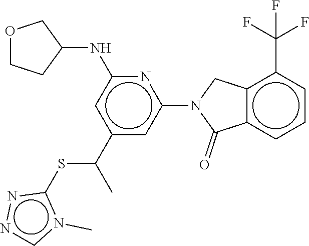 |
| 180 | 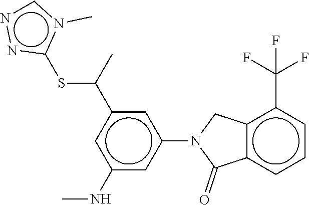 |
| 181 | 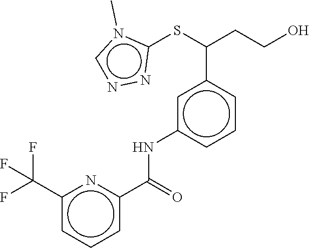 |
| 182 | 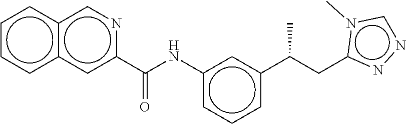 |
| 183a | 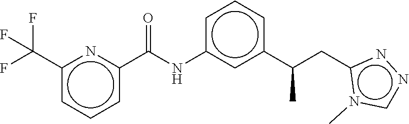 |
| 183b | 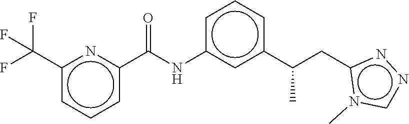 |
| 184 | 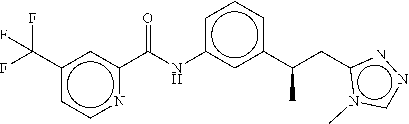 |
| 185 | 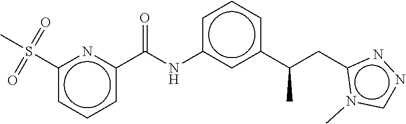 |
| 186 | 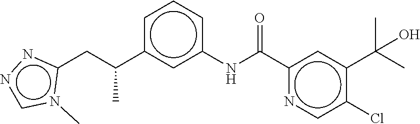 |
| 187 | 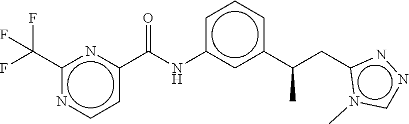 |
| 188 | 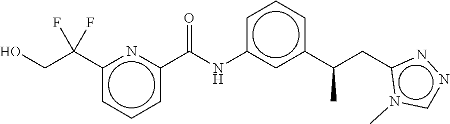 |
| 189 | 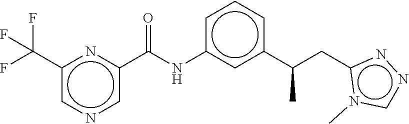 |
| 190 | 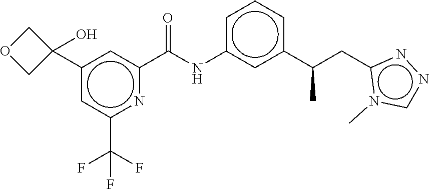 |
| 191 | 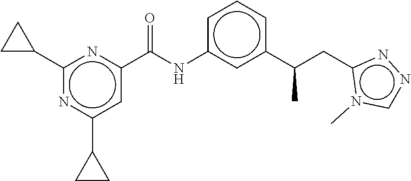 |
| 192 | 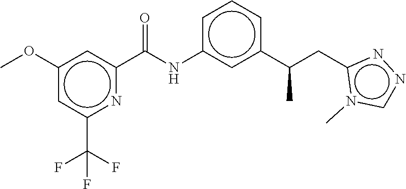 |
| 193 | 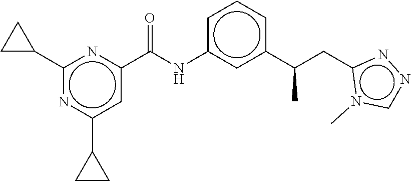 |
| 194 | 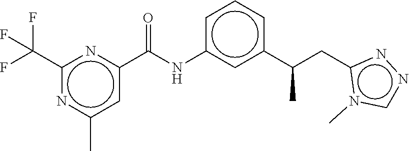 |
| 195 | 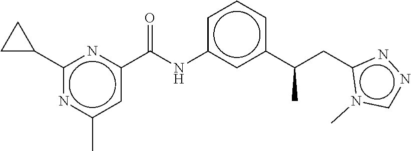 |
| 196 | 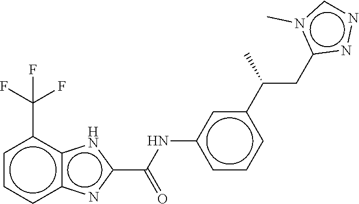 |
| 197 | 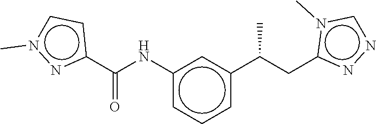 |
| 198 | 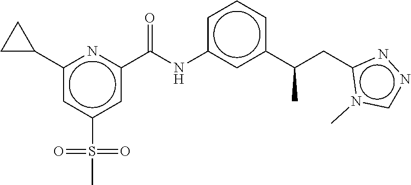 |
| 198b | 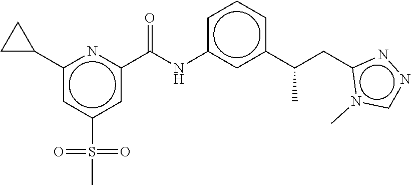 |
| 199 | 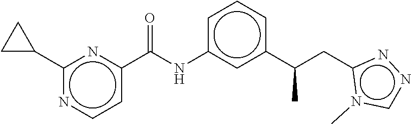 |
| 200 | 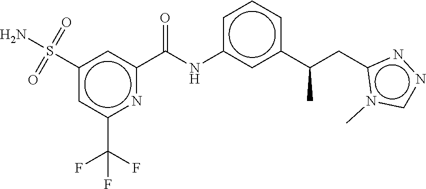 |
| 201 | 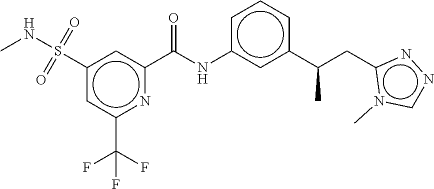 |
| 202 | 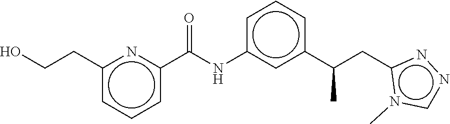 |
| 203 | 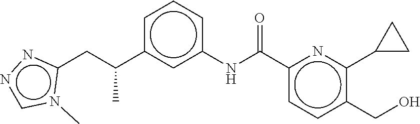 |
| 204 | 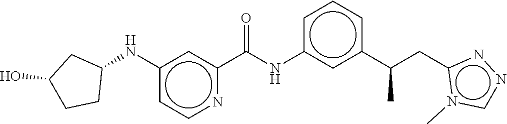 |
| 205 | 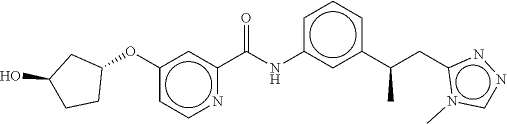 |
| 206 | 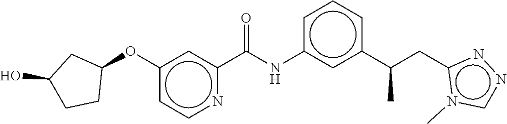 |
| 207 | 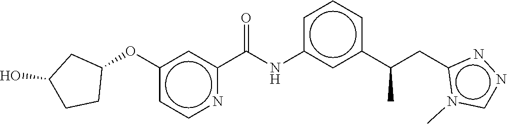 |
| 208 | 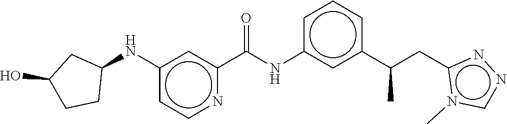 |
| 209 | 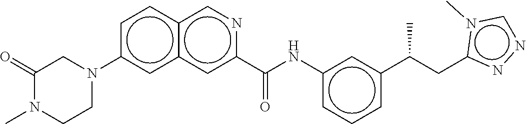 |
| 210 | 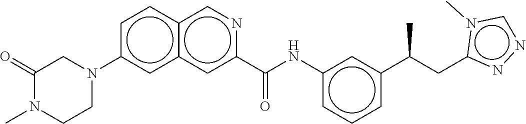 |
| 211 | 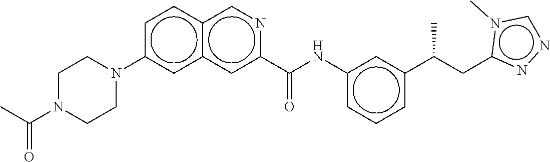 |
| 212 | 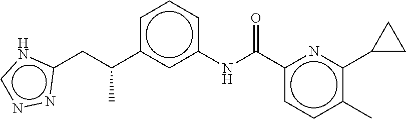 |
| 213a | 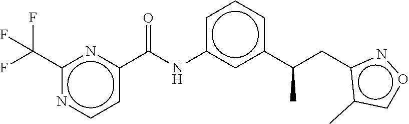 |
| 213b | 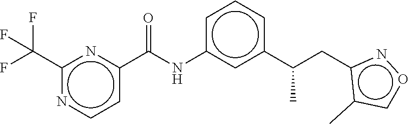 |
| 214 | 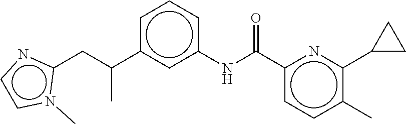 |
| 215 | 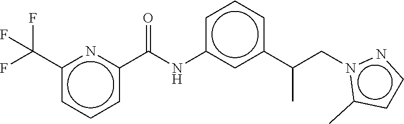 |
| 216 | 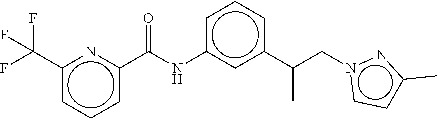 |
| 217 | 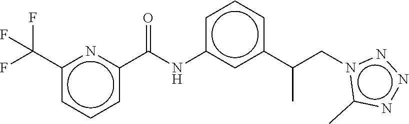 |
| 218 | 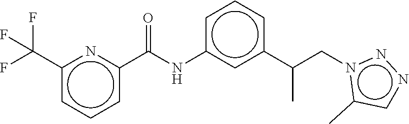 |
| 219 | 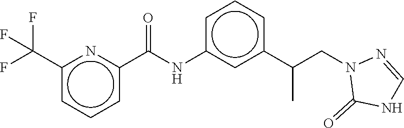 |
| 220 | 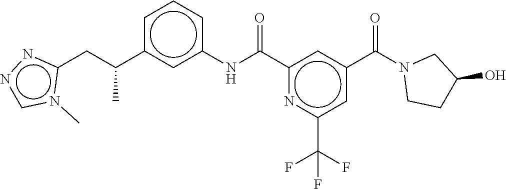 |
| 221 | 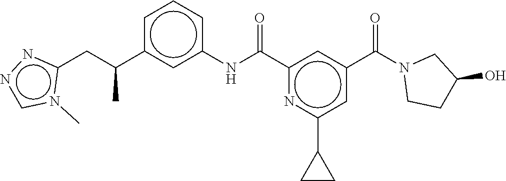 |
| 222 | 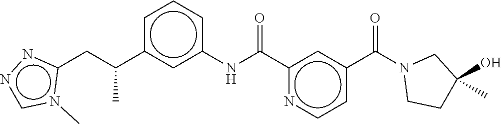 |
| 223 | 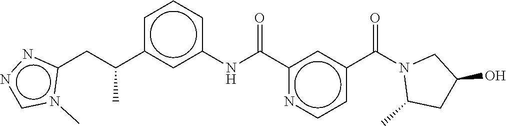 |
| 224 | 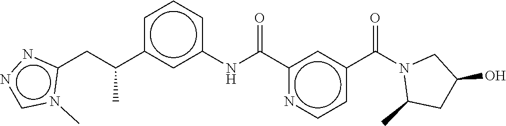 |
| 225 | 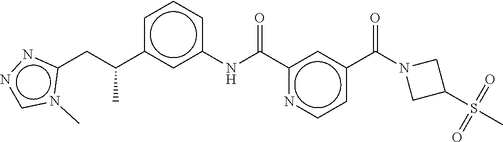 |
| 226 | 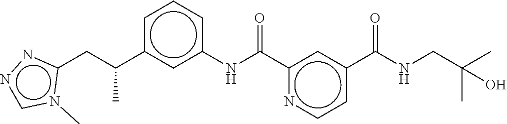 |
| 227 | 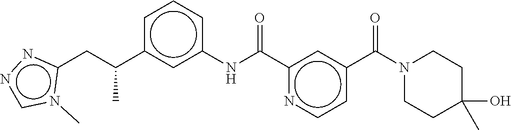 |
| 228 | 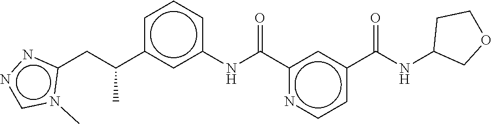 |
| 229 | 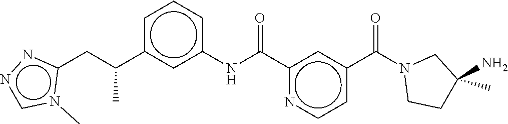 |
| 230 | 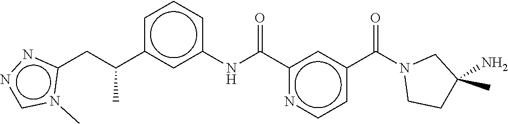 |
| 231 | 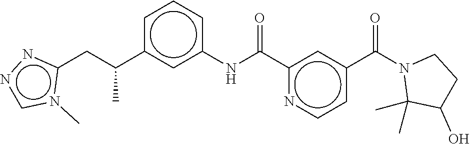 |
| 232 | 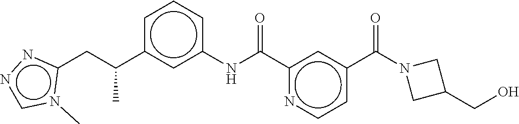 |
| 233 | 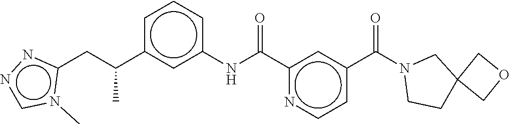 |
| 234 | 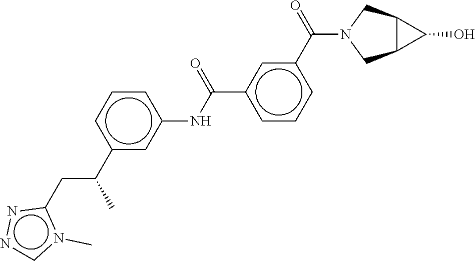 |
| 235 | 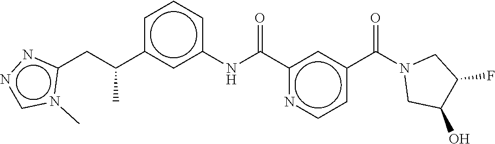 |
| 236 | 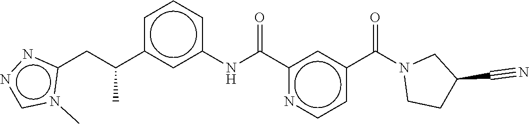 |
| 237 | 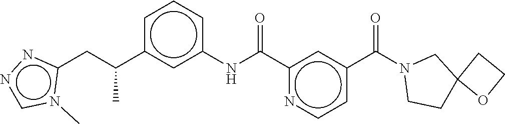 |
| 238 | 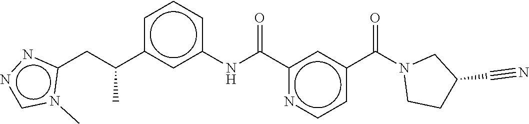 |
| 239 | 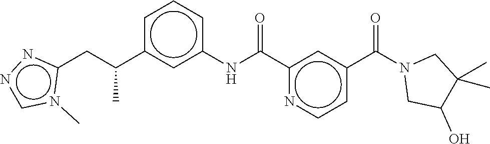 |
| 240 | 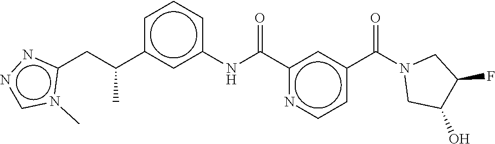 |
| 241 | 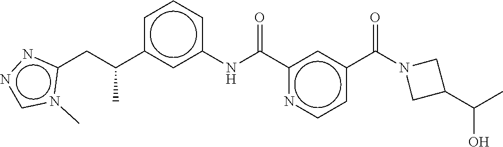 |
| 242 | 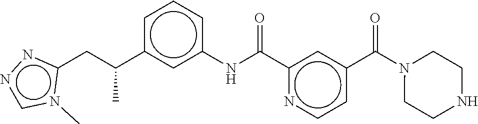 |
| 243 | 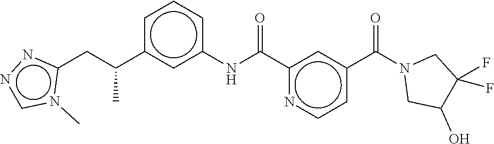 |
| 244 | 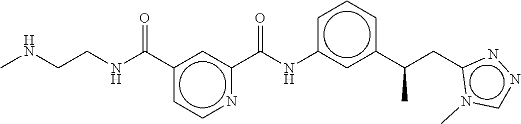 |
| 245 | 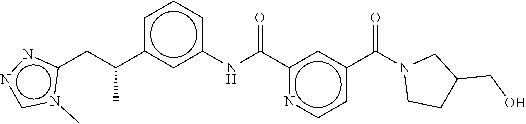 |
| 246 | 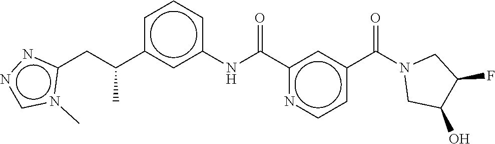 |
| 247 | 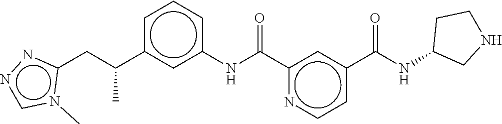 |
| 248 | 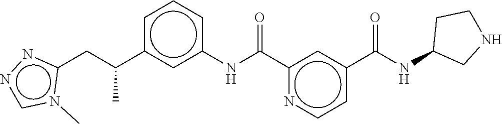 |
| 249 | 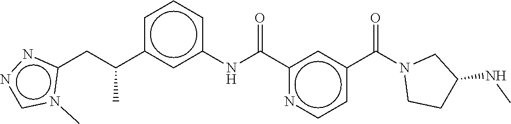 |
| 250 | 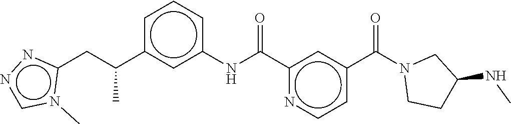 |
| 251 | 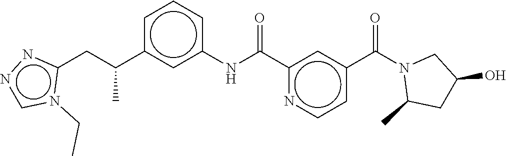 |
| 252 | 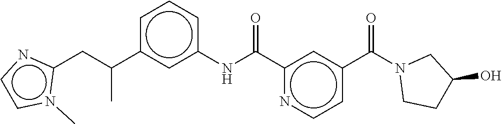 |
| 252a | 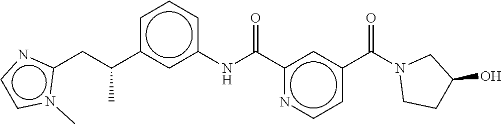 |
| 253 | 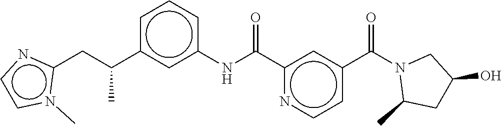 |
| 254 | 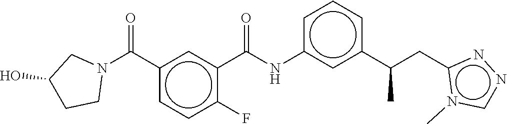 |
| 255a | 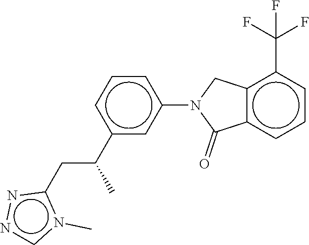 |
| 255b | 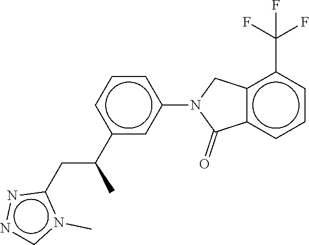 |
| 256 | 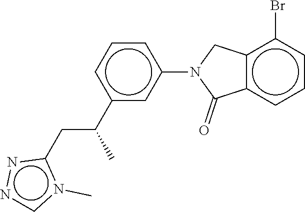 |
| 257 | 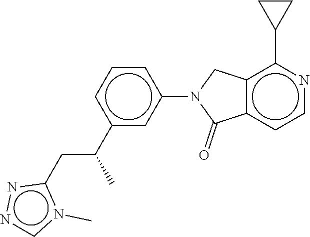 |
| 258 | 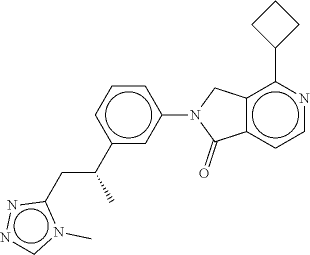 |
| 259 | 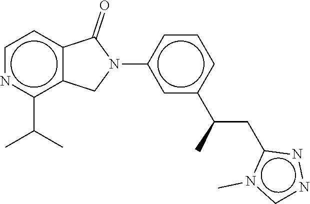 |
| 260 | 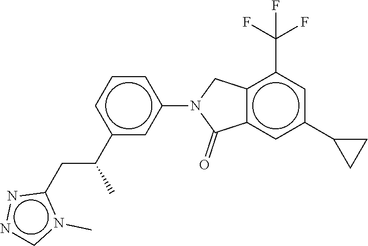 |
| 261 | 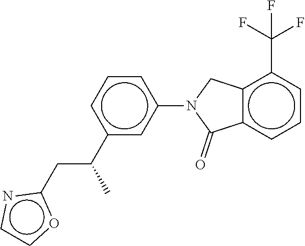 |
| 262 | 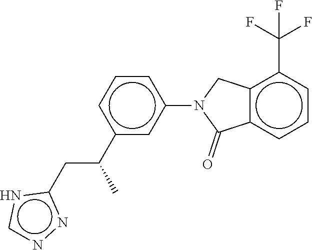 |
| 263 | 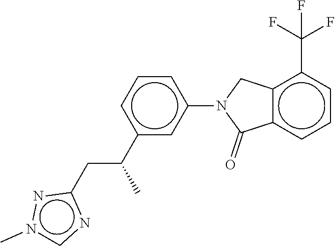 |
| 264 | 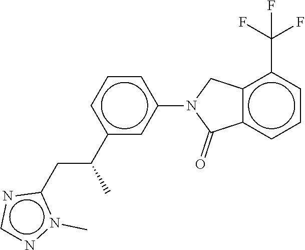 |
| 265 | 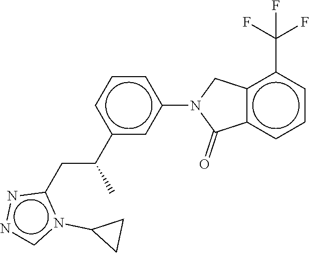 |
| 266 | 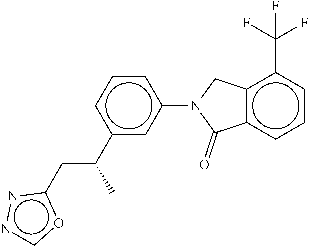 |
| 267 | 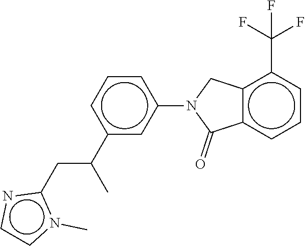 |
| 267a | 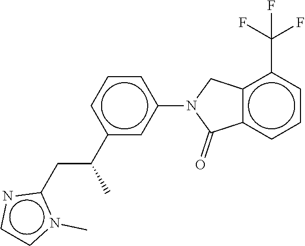 |
| 268 | 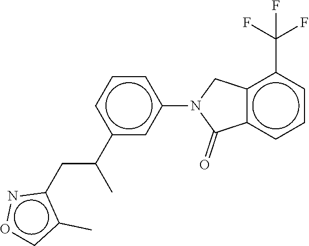 |
| 269 | 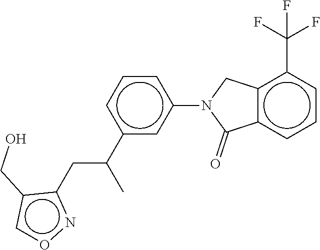 |
| 270a | 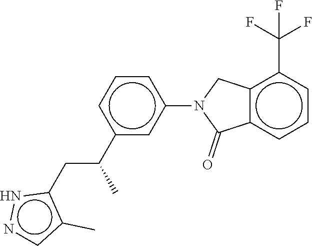 |
| 270b | 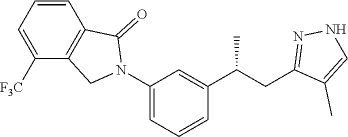 [SMILES: Cc1c[nH]nc1C[C@@H](C)c1cccc(N2Cc3c(cccc3C(F)(F)F)C2=O)c1] |
| 271 | 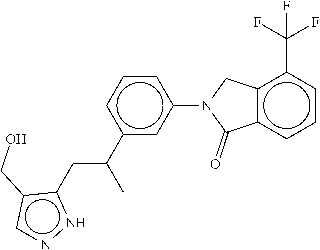 |
| 271a | 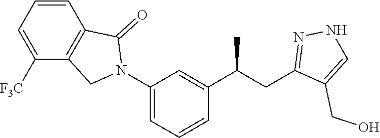 [SMILES: C[C@@H](Cc1n[nH]cc1CO)c1cccc(N2Cc3c(cccc3C(F)(F)F)C2=O)c1] |
| 271b | 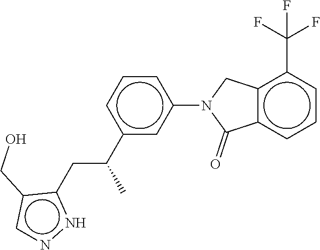 |
| 272 | 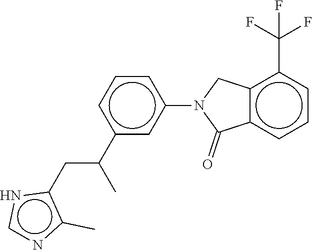 |
| 272a | 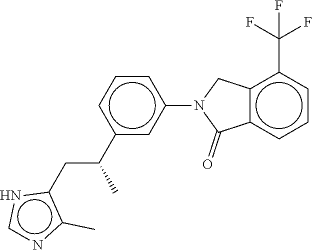 |
| 273 | 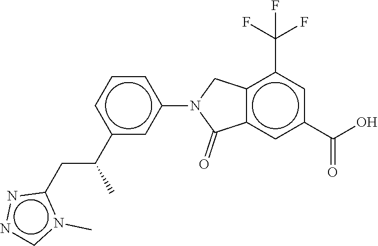 |
| 274 | 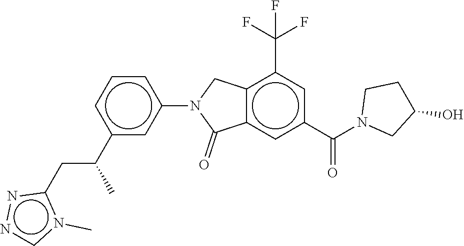 |
| 275 | 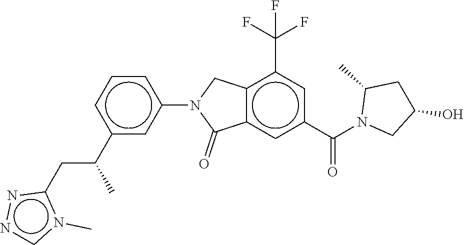 |
| 276 | 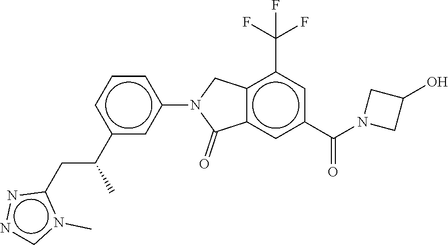 |
| 277 | 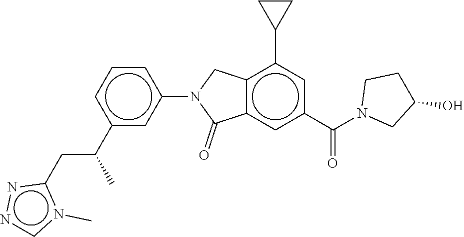 |
| 278 | 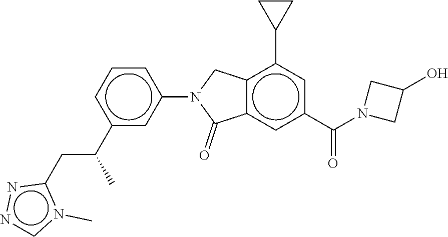 |
| 279 | 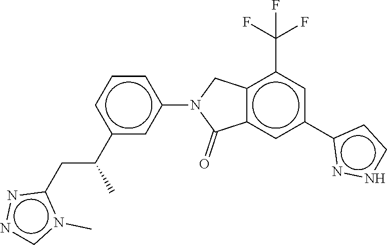 |
| 280 | 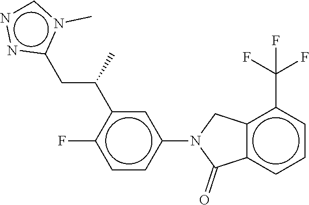 |
| 281 | 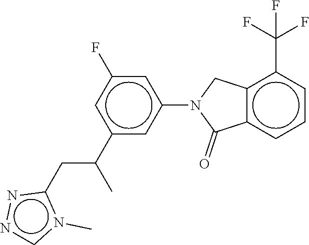 |
| 281a | 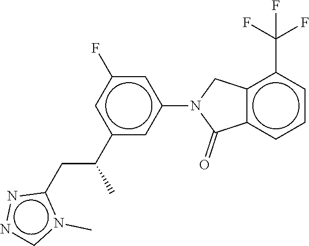 |
| 281b | 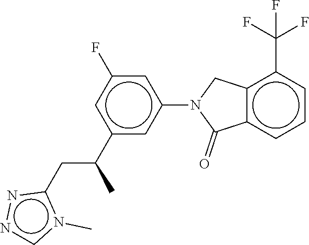 |
| 282 | 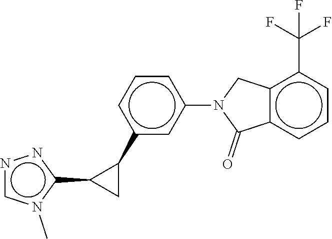 |
| 283 | 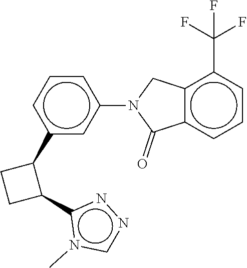 |
| 283a | 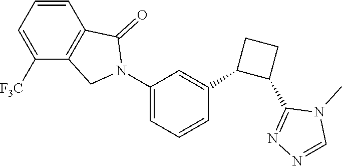 [SMILES: Cn1cnnc1[C@H]1CC[C@H]1c1cccc(N2Cc3c(cccc3C(F)(F)F)C2=O)c1] |
| 283b | 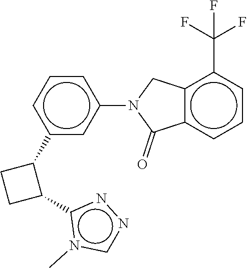 |
| 284 | 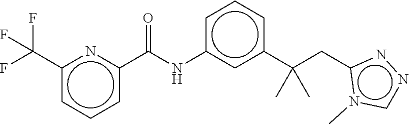 |
| 285 | 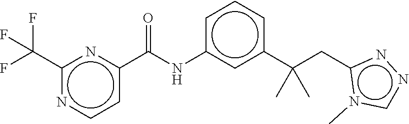 |
| 286 | 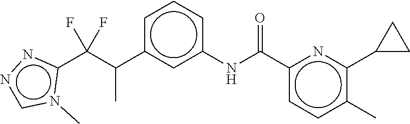 |
| 287 | 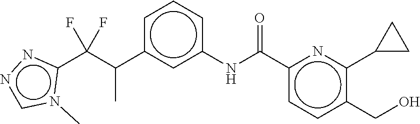 |
| 288 | 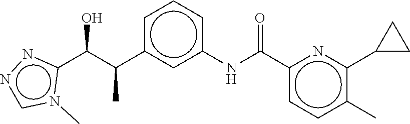 |
| 289 | 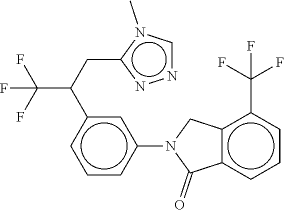 |
| 290 | 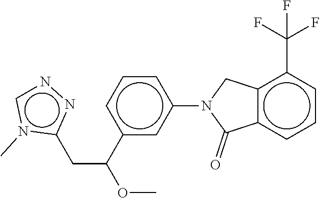 |
| 291 | 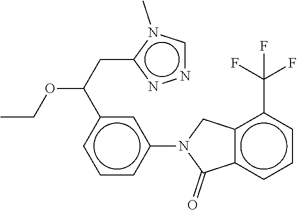 |
| 292 | 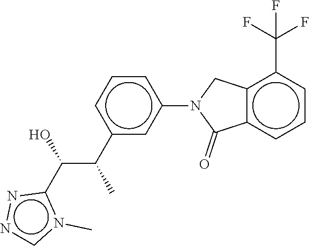 |
| 293 | 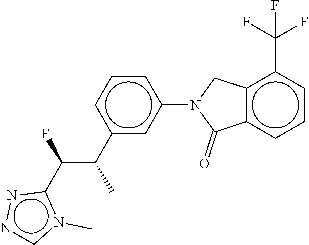 |
| 294 | 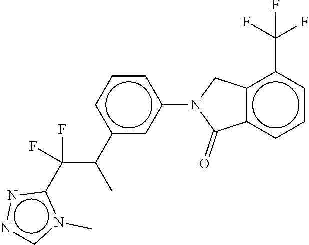 |
| 295 | 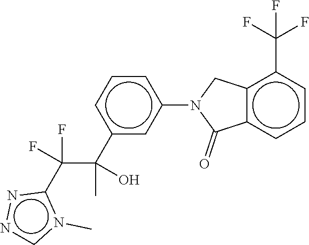 |
| 296 | 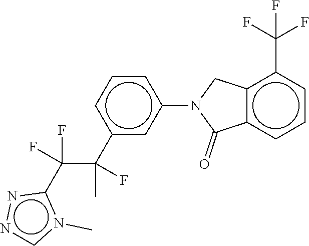 |
| 296a | 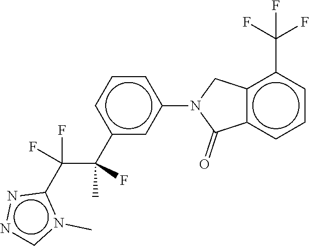 |
| 296b | 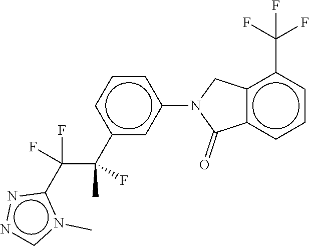 |
| 297 | 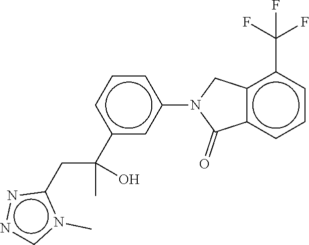 |
| 298 | 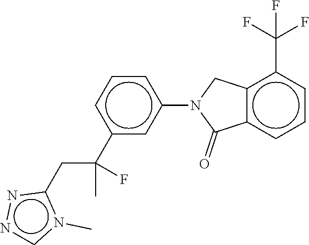 |
| 298a | 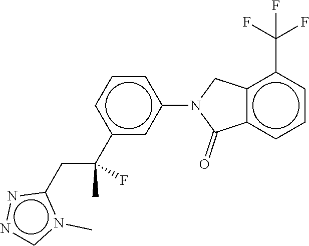 |
| 298b | 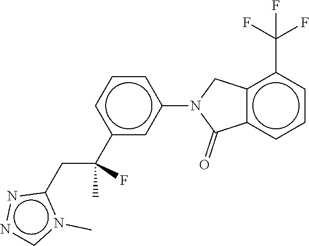 |
| 299 | 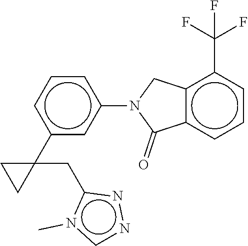 |
| 300 | 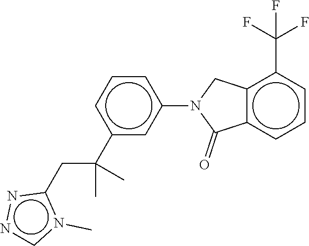 |
| 301 | 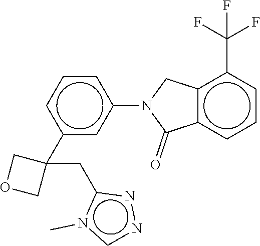 |
| 302 | 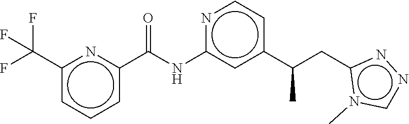 |
| 303 | 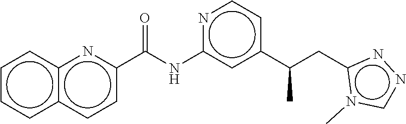 |
| 304a | 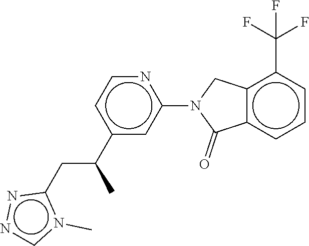 |
| 304b | 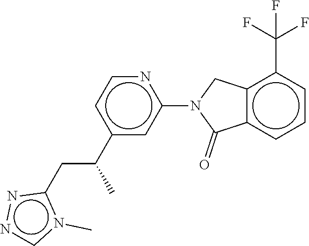 |
| 305 | 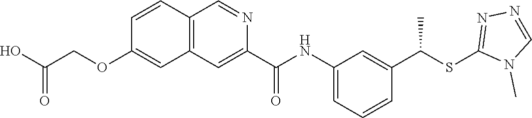 [SMILES: C[C@H](Sc1nncn1C)c1cccc(NC(=O)c2cc3cc(OCC(=O)O)ccc3cn2)c1] |
| 306a | 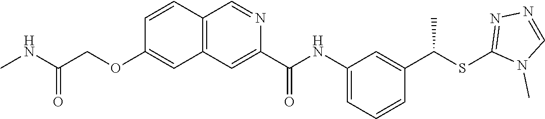 [SMILES: CNC(=O)COc1ccc2cnc(C(=O)Nc3cccc([C@H](C)Sc4nncn4C)c3)cc2c1] |
| 306b | 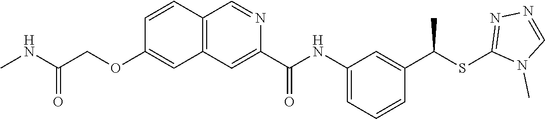 [SMILES: CNC(=O)COc1ccc2cnc(C(=O)Nc3cccc([C@@H](C)Sc4nncn4C)c3)cc2c1] |
| 307 | 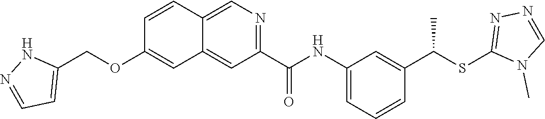 [SMILES: C[C@H](Sc1nncn1C)c1cccc(NC(=O)c2cc3cc(OCc4ccn[nH]4)ccc3cn2)c1] |
| 308 | 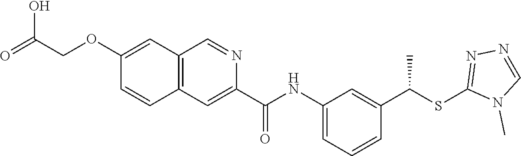 [SMILES: C[C@H](Sc1nncn1C)c1cccc(NC(=O)c2cc3ccc(OCC(=O)O)cc3cn2)c1] |
| 309 | 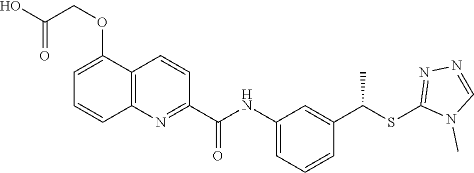 [SMILES: C[C@H](Sc1nncn1C)c1cccc(NC(=O)c2ccc3c(OCC(=O)O)cccc3n2)c1] |
| 310 | 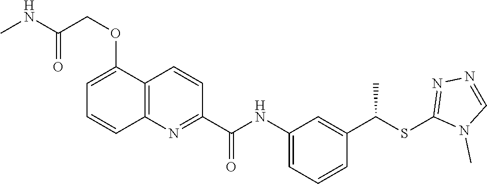 [SMILES: CNC(=O)COc1cccc2nc(C(=O)Nc3cccc([C@H](C)Sc4nncn4C)c3)ccc12] |
| 311 | 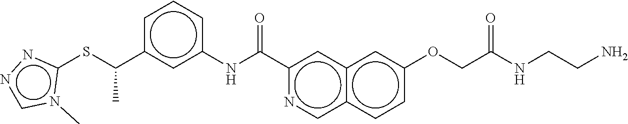 |
| 312a | 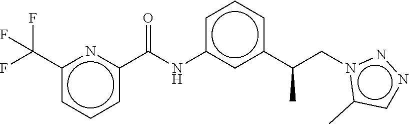 |
| 312b | 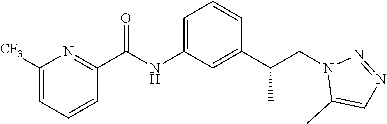 [SMILES: Cc1cnnn1C[C@H](C)c1cccc(NC(=O)c2cccc(C(F)(F)F)n2)c1] |
| 313 | 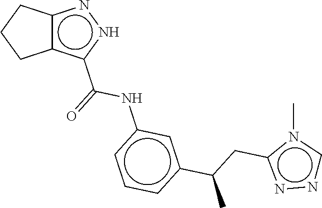 |
| 314 | 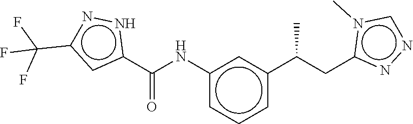 |
| 315 | 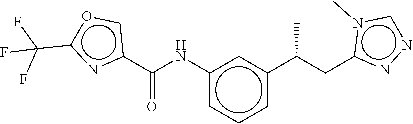 |
| 316 | 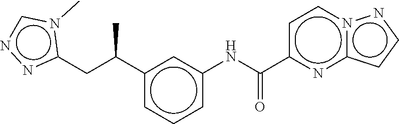 |
| 317 | 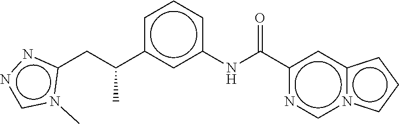 |
| 318 | 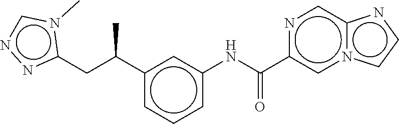 |
| 319 | 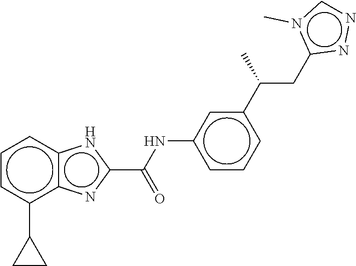 |
| 320 | 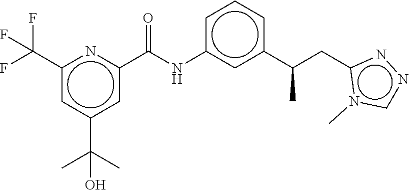 |
| 321 | 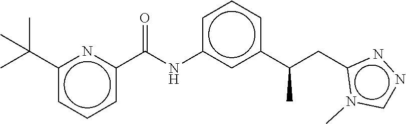 |
| 322 | 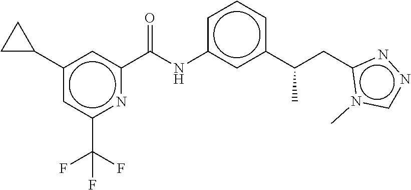 |
| 323 | 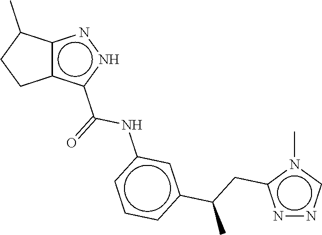 |
| 324 | 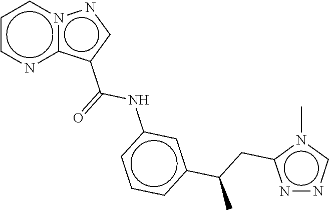 |
| 325 | 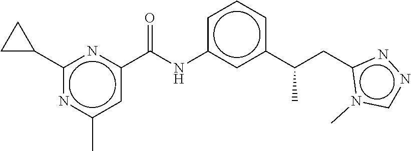 |
| 326 | 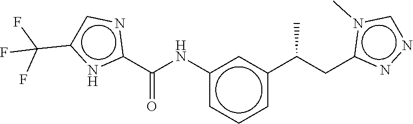 |
| 327 | 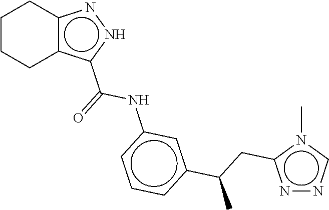 |
| 328 | 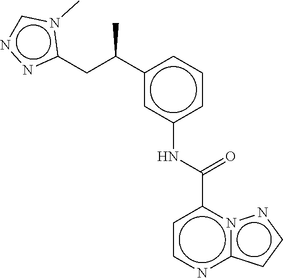 |
| 329 | 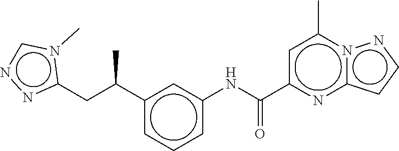 |
| 330 | 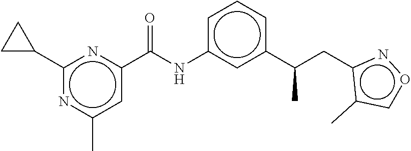 |
| 331 | 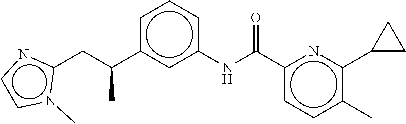 |
| 332 | 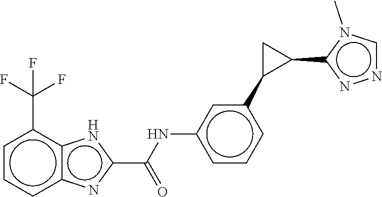 |
| 333 | 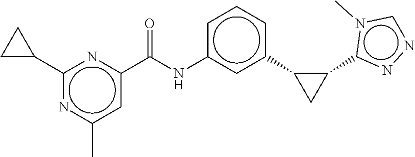 |
| 334 | 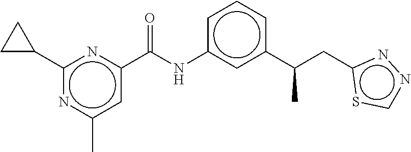 |
| 335 | 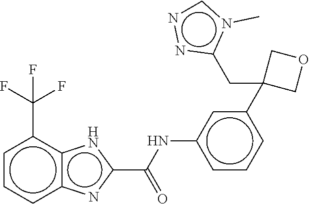 |
| 336 | 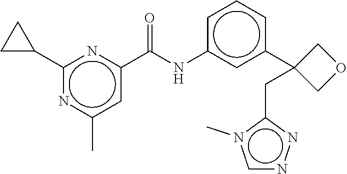 |
| 337 | 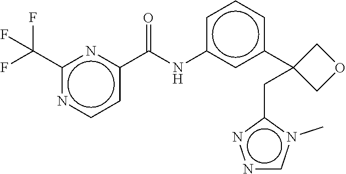 |
| 338 | 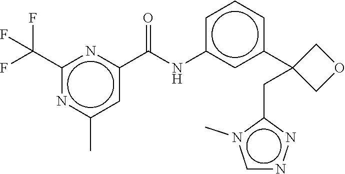 |
| 339 | 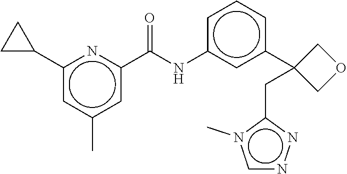 |
| 340 | 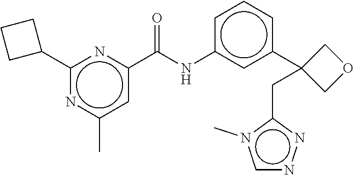 |
| 341 | 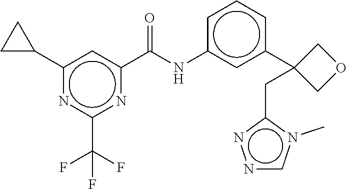 |
| 342 | 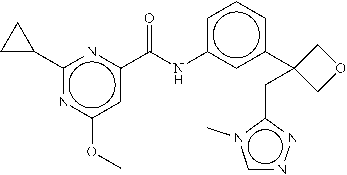 |
| 343 | 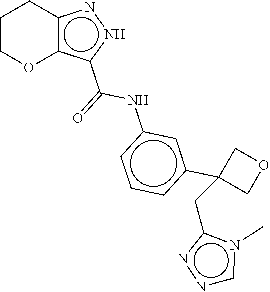 |
| 344 | 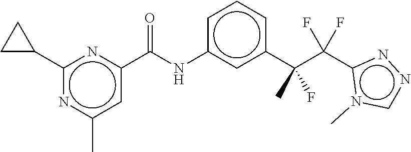 |
| 345 | 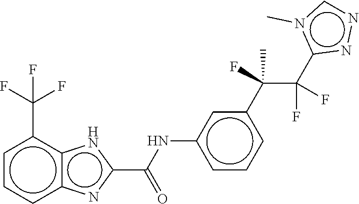 |
| 346 | 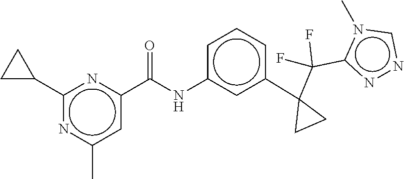 |
| 347 | 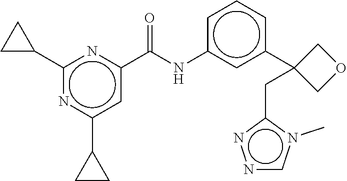 |
| 348 | 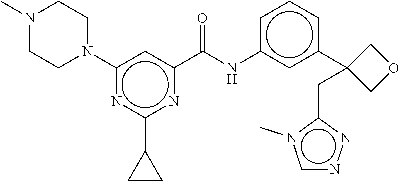 |
| 349 | 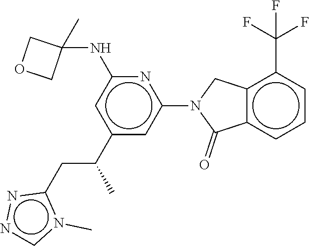 |
| 350 | 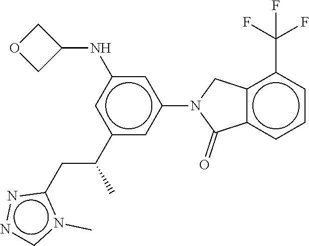 |
| 351 | 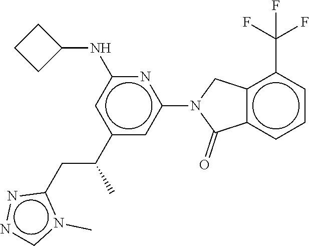 |
| 352 | 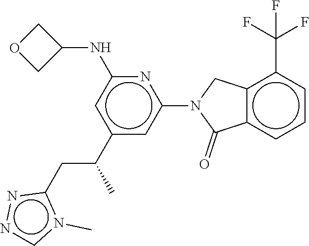 |
| 353 | 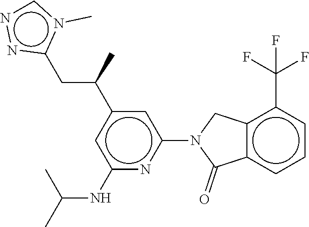 |
| 354 | 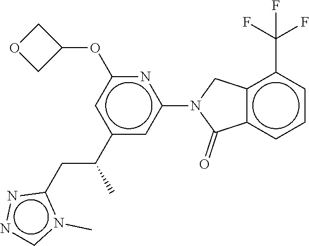 |
| 355 | 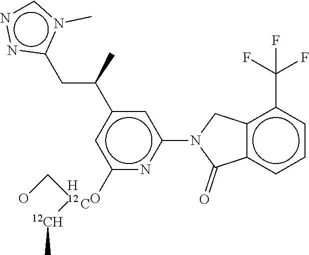 |
| 356 | 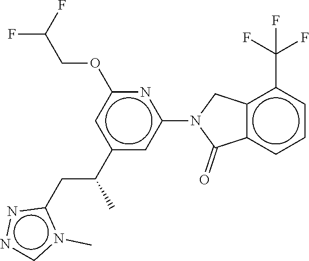 |
| 357 | 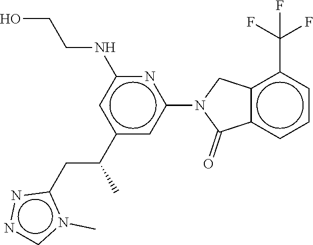 |
| 358 | 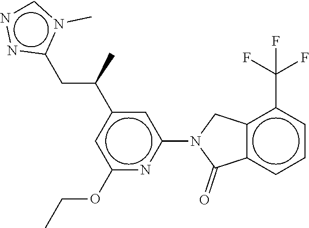 |
| 359 | 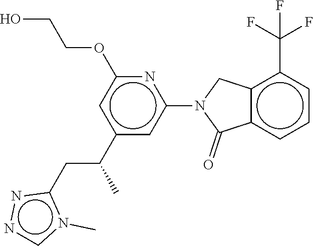 |
| 360 | 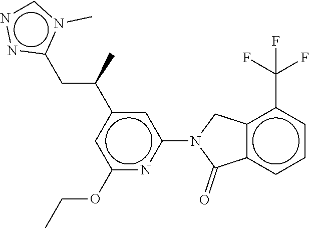 |
| 361 | 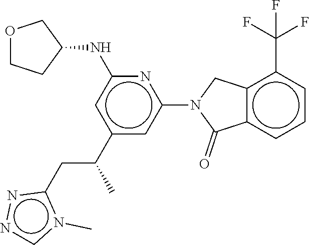 |
| 362 | 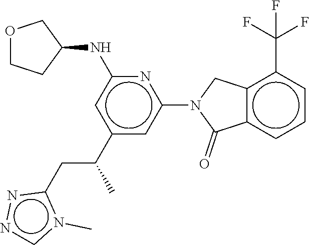 |
| 363 | 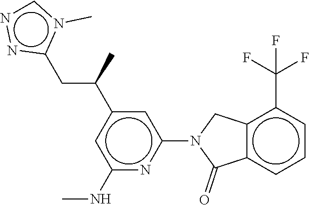 |
| 364 | 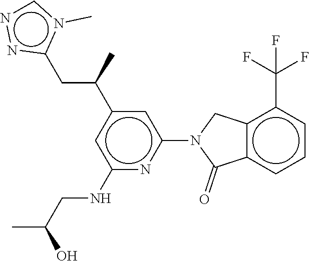 |
| 365 | 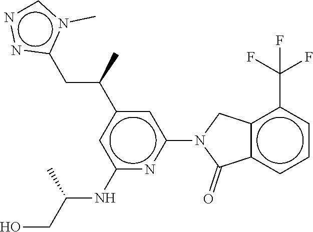 |
| 366 | 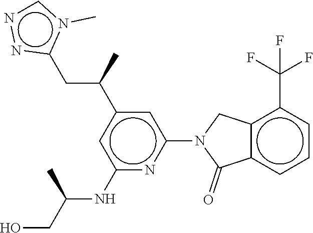 |
| 367a | 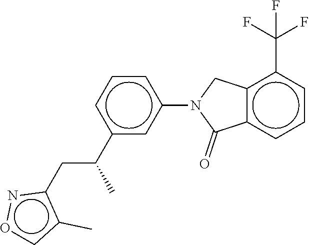 |
| 367b | 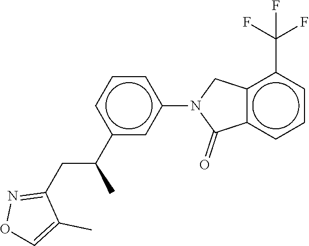 |
| 368 | 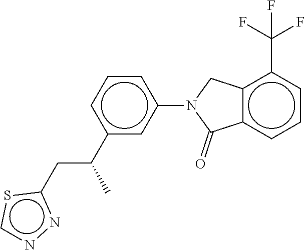 |
| 369 | 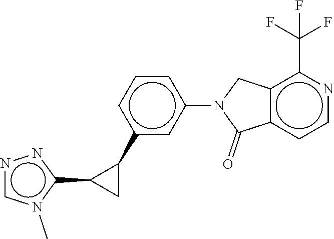 |
| 370 | 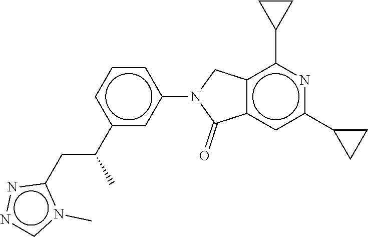 |
| 371 | 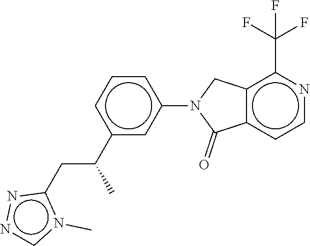 |
| 372 | 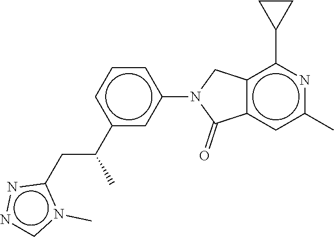 |
| 373 | 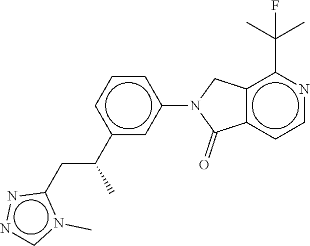 |
| 374 | 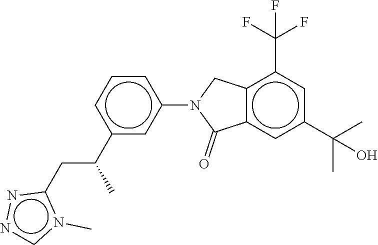 |
| 375 | 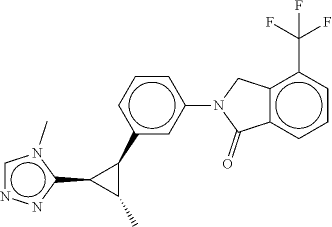 |
| 376 | 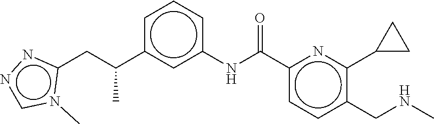 |
| 377 | 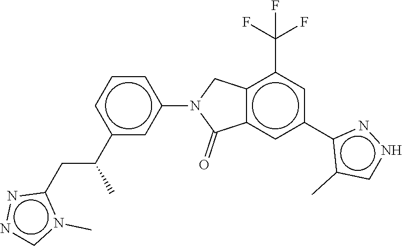 |
| 378 | 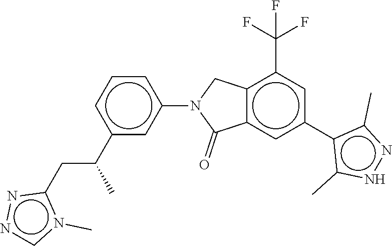 |
| 379 | 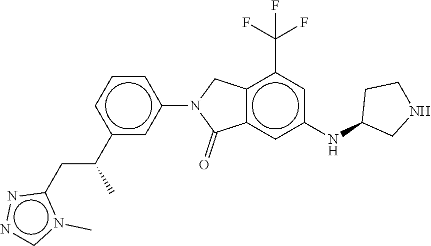 |
| 380 | 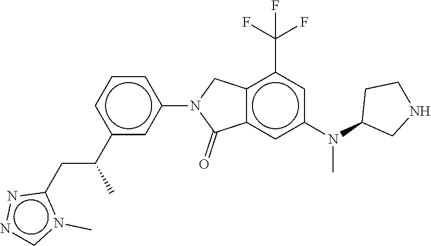 |
| 381 | 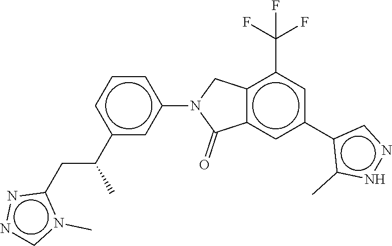 |
| 382 | 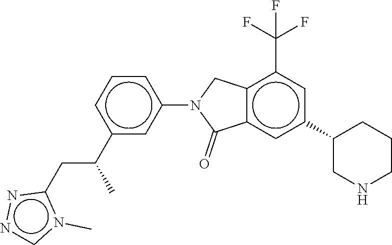 |
| 383 | 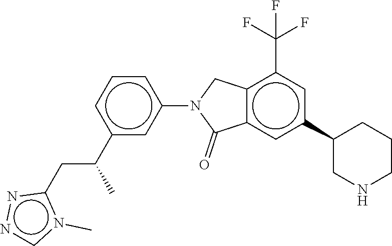 |
| 384 | 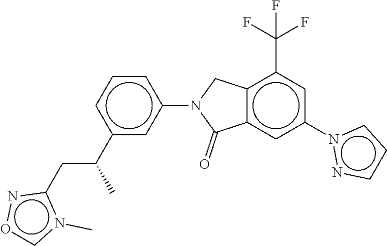 |
| 385 | 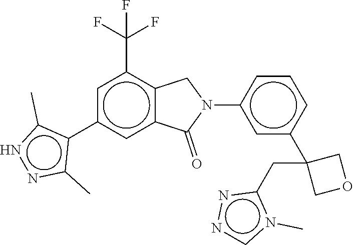 |
| 386 | 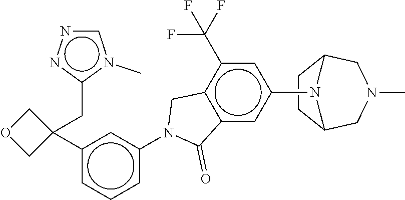 |
| 387 | 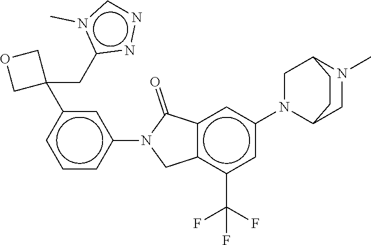 |
| 388 | 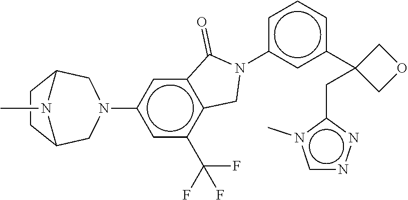 |
| 389 | 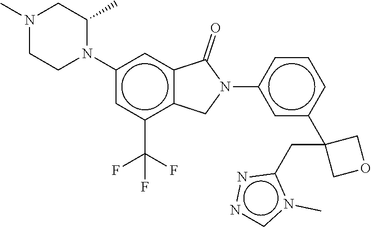 |
| 390 | 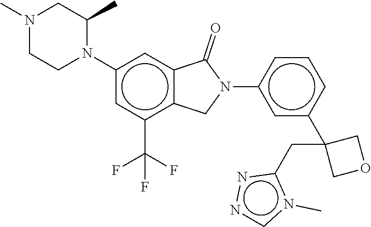 |
| 391 | 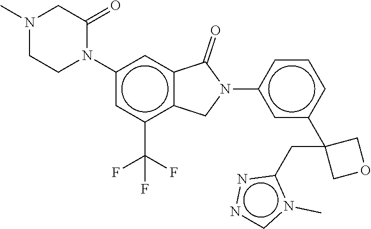 |
| 392 | 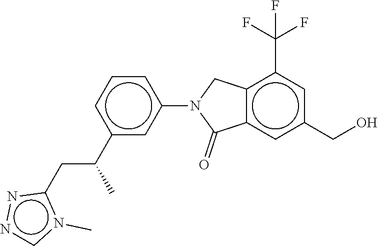 |
| 393 | 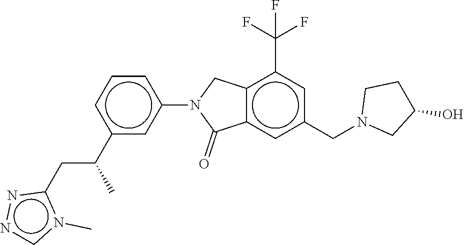 |
| 394 | 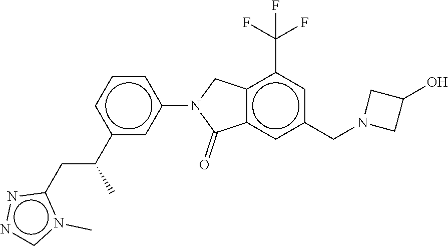 |
| 395 | 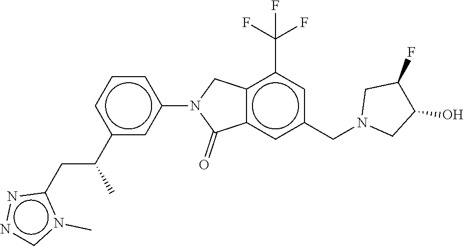 |
| 396 | 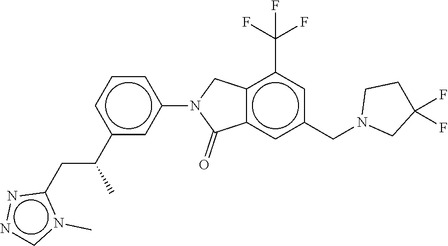 |
| 397 | 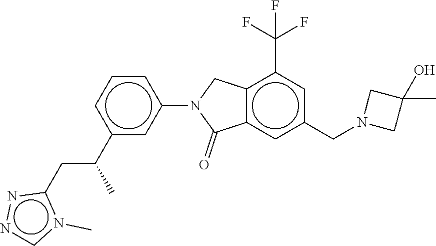 |
| 398 | 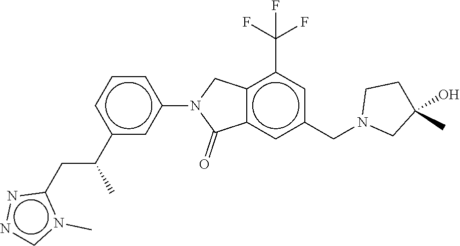 |
| 399 | 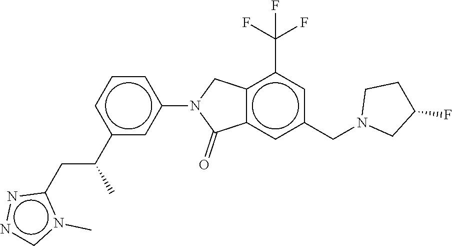 |
| 400 | 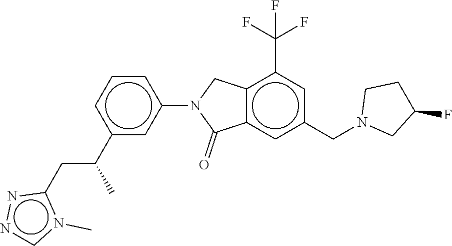 |
| 401 | 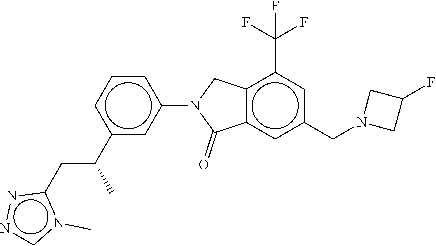 |
| 402 | 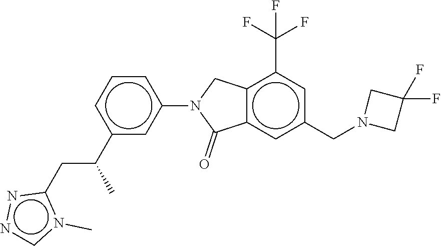 |
| 403 | 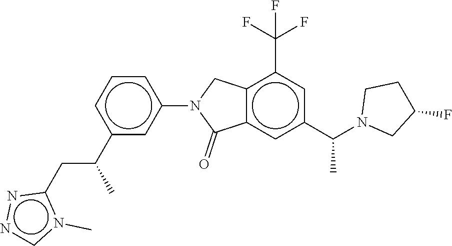 |
| 404 | 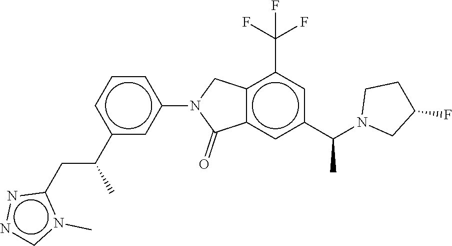 |
| 405 | 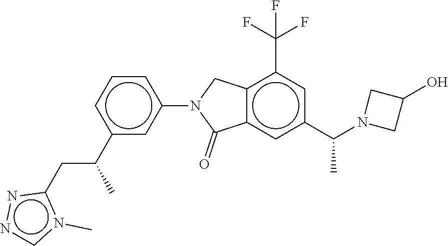 |
| 406 | 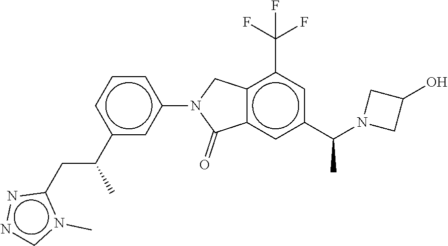 |
| 407 | 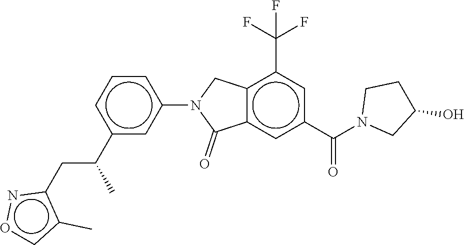 |
| 408 | 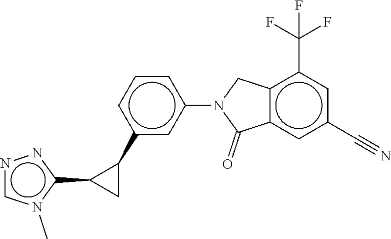 |
| 409 | 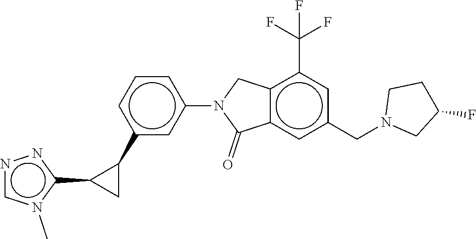 |
| 410 | 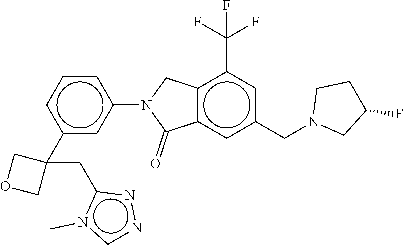 |
| 411 | 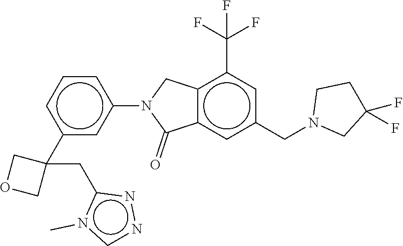 |
| 412 | 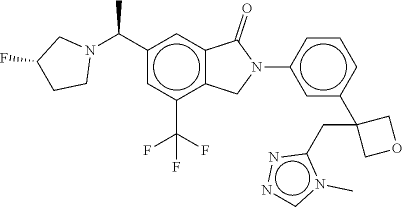 |
| 413 | 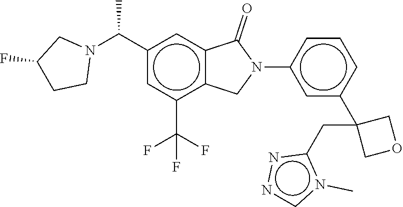 |
| 414 | 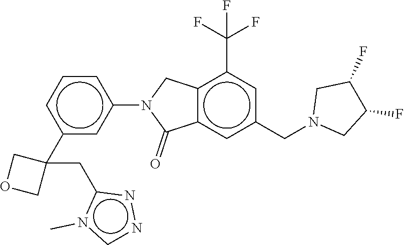 |
| 415 | 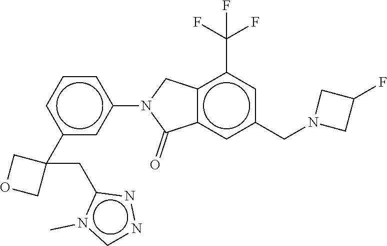 |
| 416 | 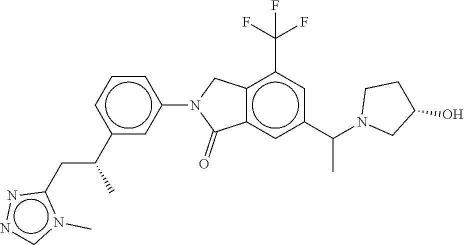 |
| 417 | 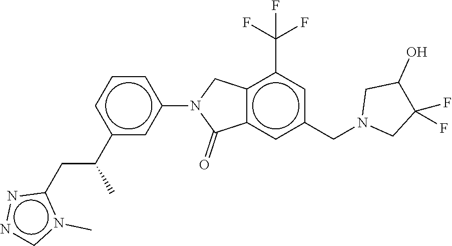 |
| 418 | 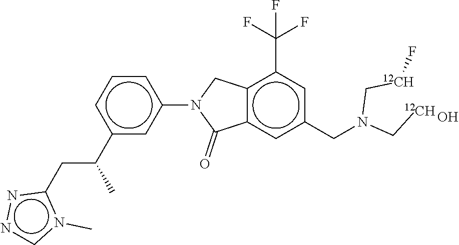 |
| 419 | 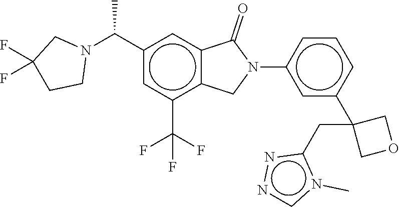 |
| 420 | 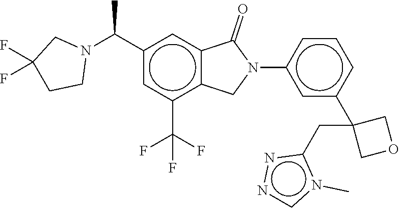 |
| 421 | 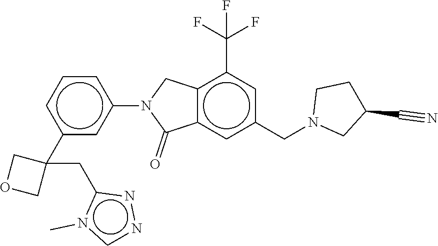 |
| 422 | 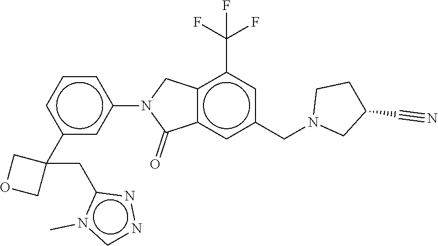 |
| 423 | 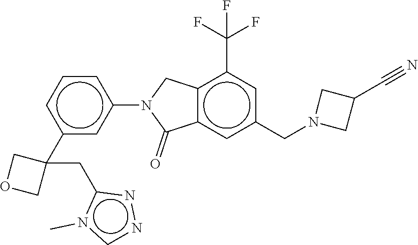 |
| 424 | 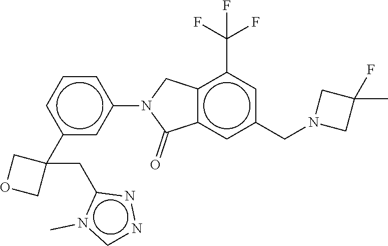 |
| 425 | 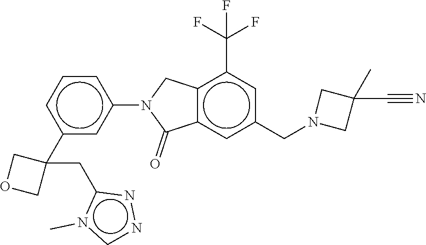 |
| 426 | 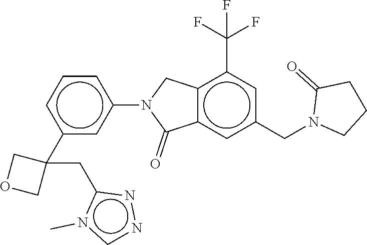 |
| 427 | 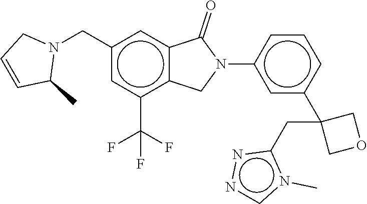 |
| 428 | 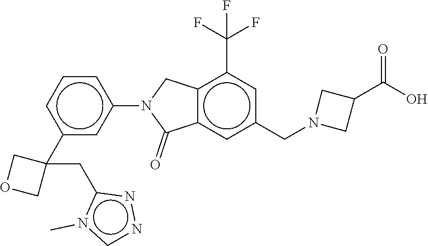 |
| 429 | 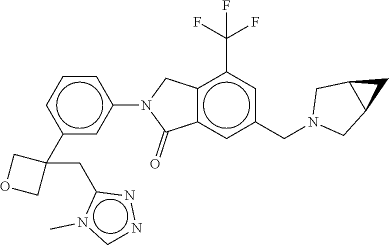 |
| 430 | 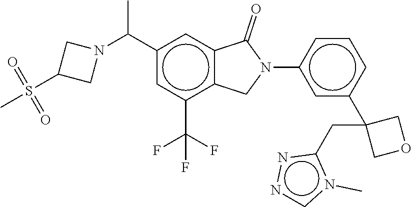 |
| 431 | 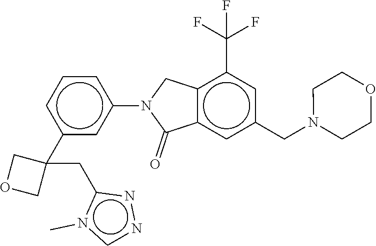 |
| 432 | 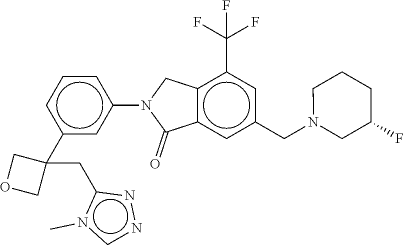 |
| 433 | 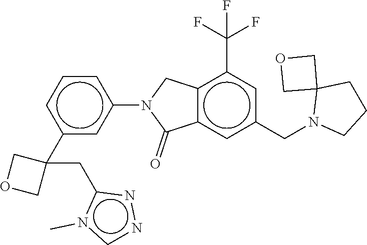 |
| 434 | 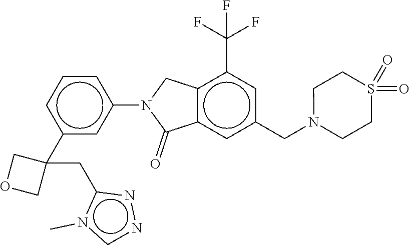 |
| 435 | 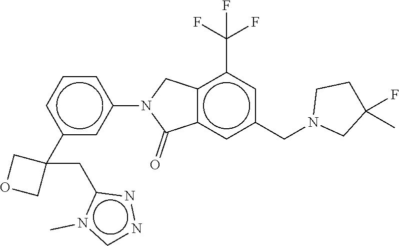 |
| 436 | 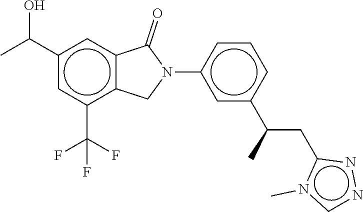 |
| 437 | 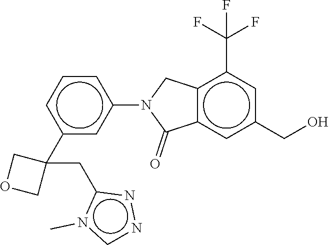 |
| 438 | 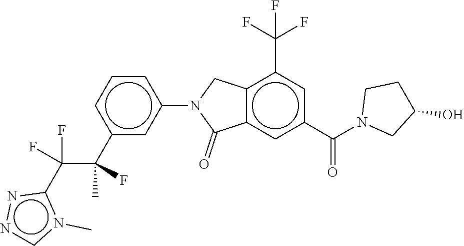 |
| 439 | 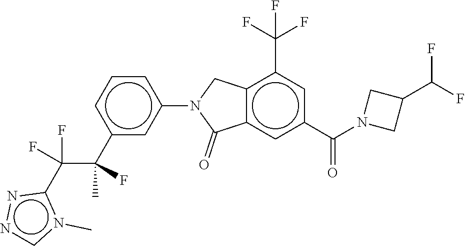 |
| 440 | 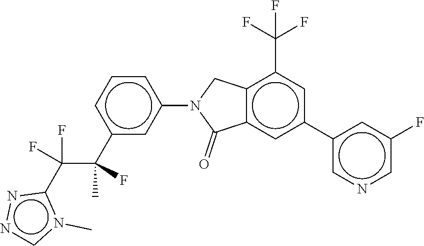 |
| 441 | 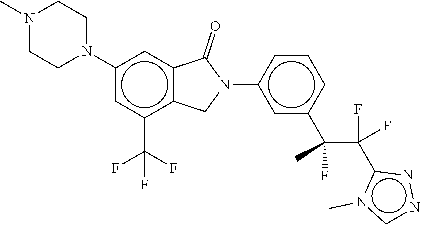 |
| 442 | 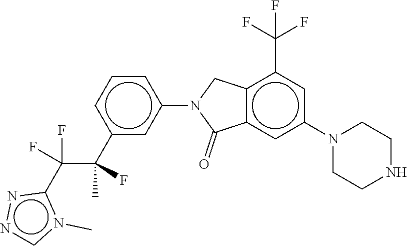 |
| 443 | 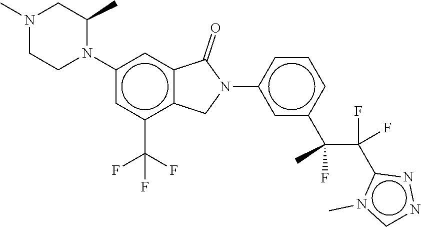 |
| 444 | 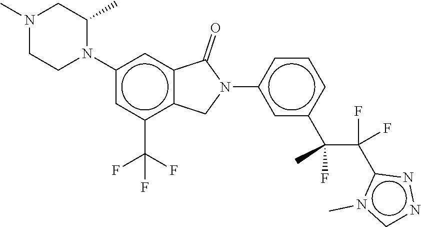 |
| 445 | 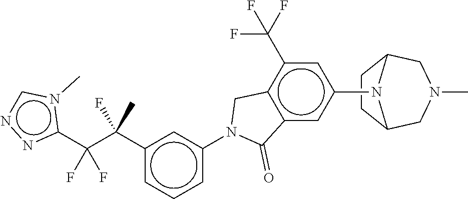 |
| 446 | 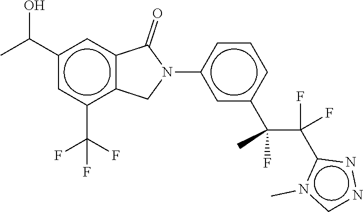 |
| 447 | 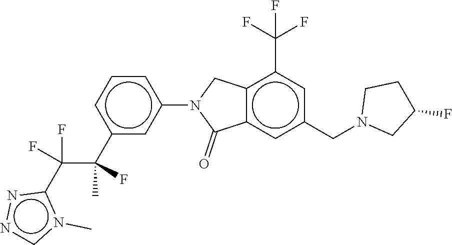 |
| 448 | 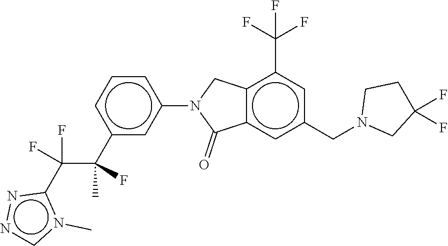 |
| 449 | 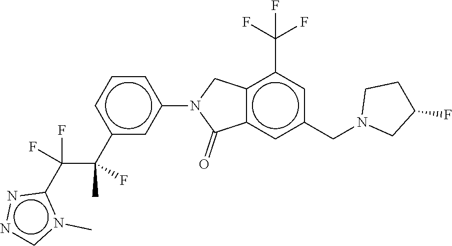 |
| 450 | 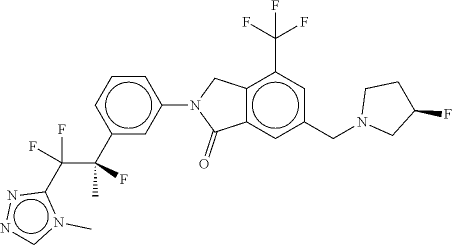 |
| 451 | 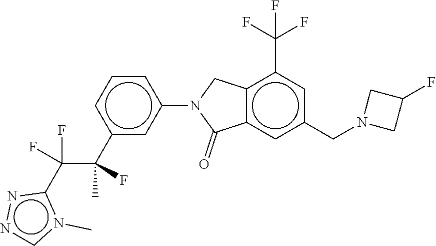 |
| 452 | 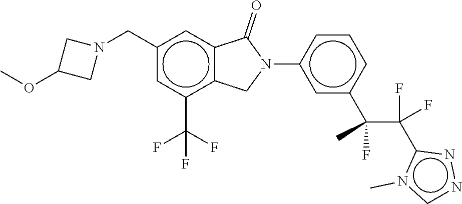 |
| 453 | 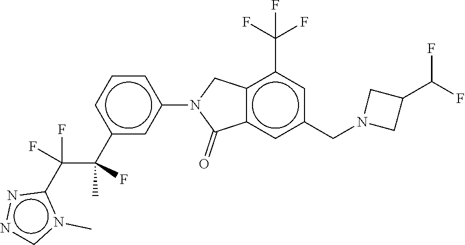 |
| 454 | 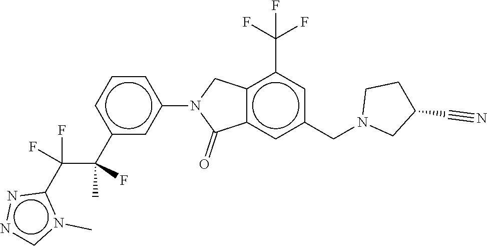 |
| 455 | 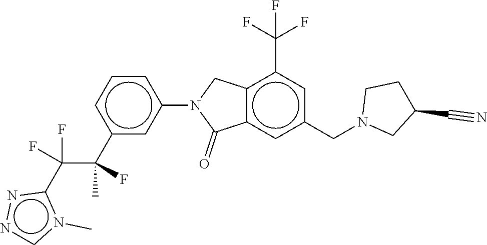 |
| 456 | 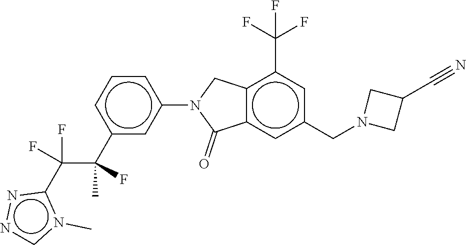 |
| 457 | 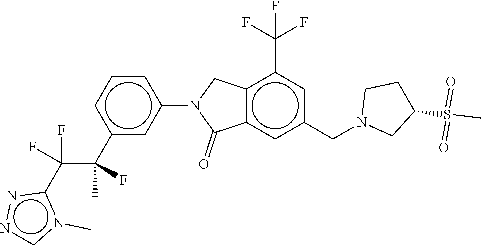 |
| 458 | 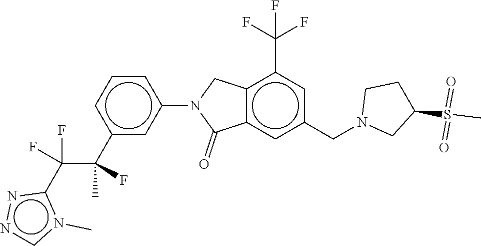 |
| 459 | 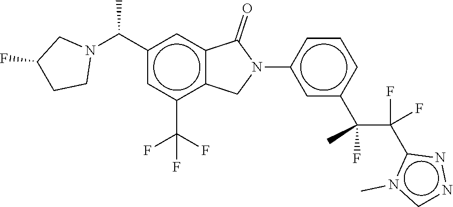 |
| 460 | 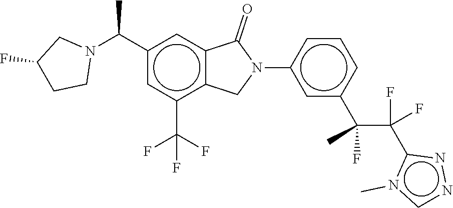 |
| 461 | 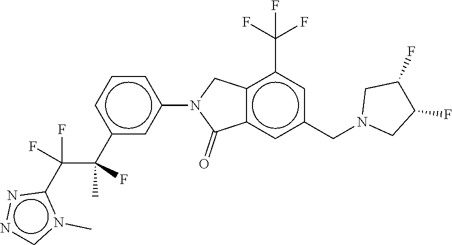 |
| 462 | 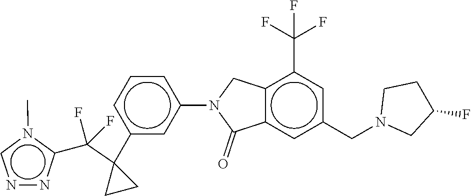 |
| 463 | 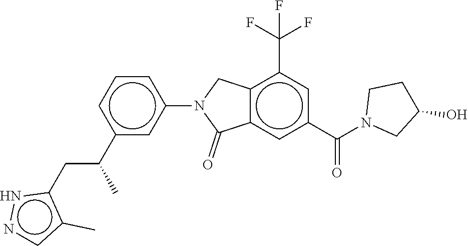 |
| 464 | 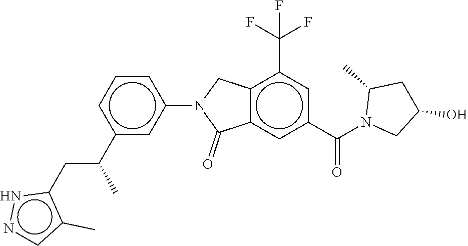 |
| 465 | 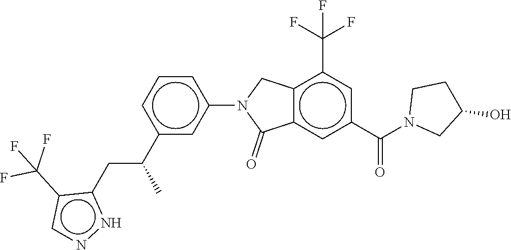 |
| 466 | 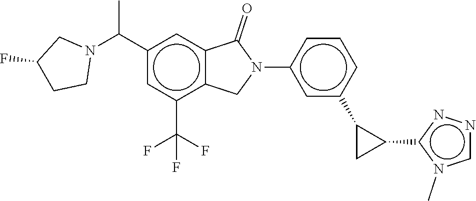 |
| 467a (467a = S isomer as shown; 467b = R isomer) | 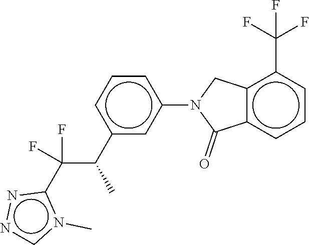 |
| 468 | 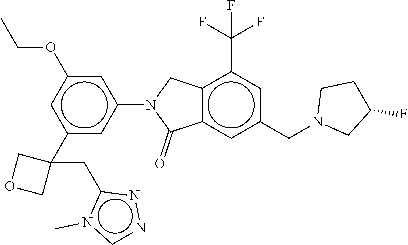 |
| 469 | 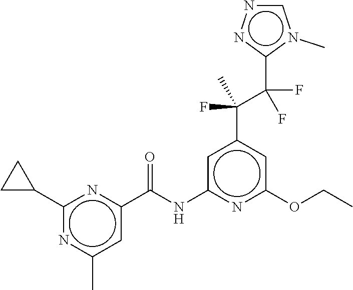 |
| 470 | 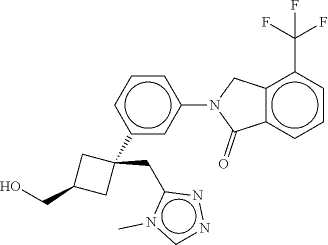 |
| 471a | 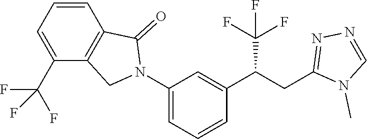 [SMILES: Cn1cnnc1C[C@H](c1cccc(N2Cc3c(cccc3C(F)(F)F)C2=O)c1)C(F)(F)F] |
| 471b | 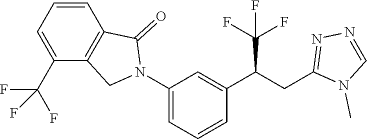 [SMILES: Cn1cnnc1C[C@@H](c1cccc(N2Cc3c(cccc3C(F)(F)F)C2=O)c1)C(F)(F)F] |
| 472 | 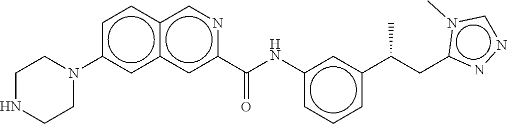 |
| 473 | 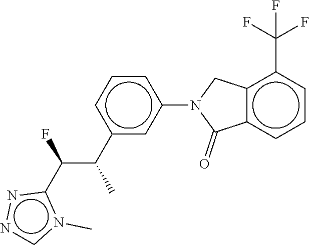 |
| 474 | 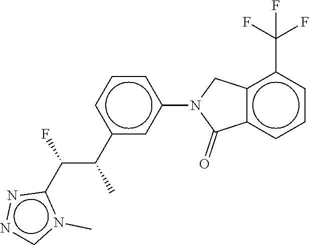 |
| 475 | 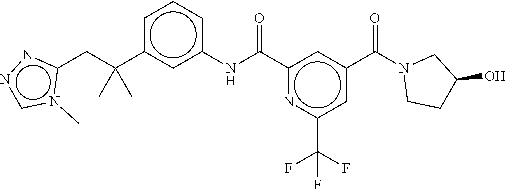 |
| 476 | 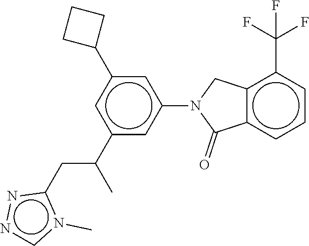 |
| 477 | 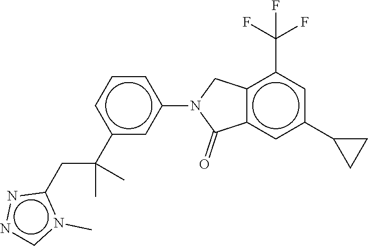 |
| 478a | 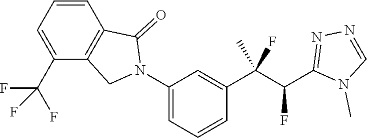 [SMILES: Cn1cnnc1[C@@H](F)[C@](C)(F)c1cccc(N2Cc3c(cccc3C(F)(F)F)C2=O)c1] |
| 478b | 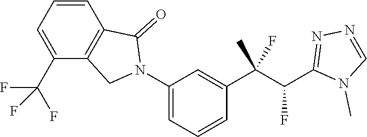 [SMILES: Cn1cnnc1[C@H](F)[C@@](C)(F)c1cccc(N2Cc3c(cccc3C(F)(F)F)C2=O)c1] |
| 479 | 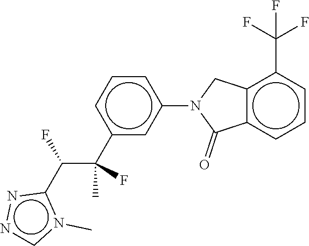 |
| 480 | 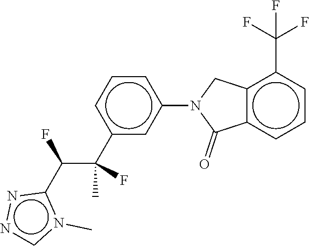 |
| 481a | 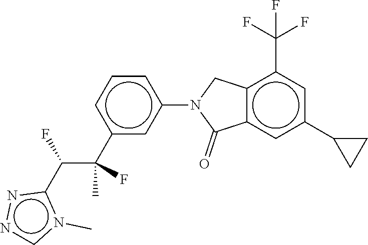 |
| 481b | 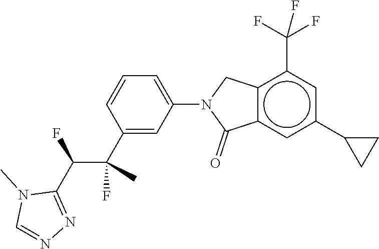 |
| 482 | 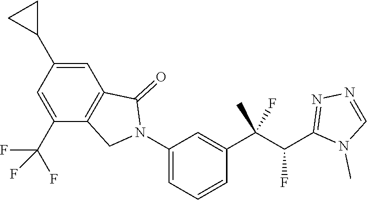 [SMILES: Cn1cnnc1[C@H](F)[C@@](C)(F)c1cccc(N2Cc3c(cc(C4CC4)cc3C(F)(F)F)C2=O)c1] |
| 483 | 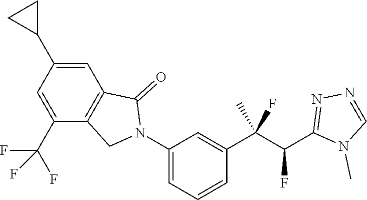 [SMILES: Cn1cnnc1[C@@H](F)[C@](C)(F)c1cccc(N2Cc3c(cc(C4CC4)cc3C(F)(F)F)C2=O)c1] |
| 484 | 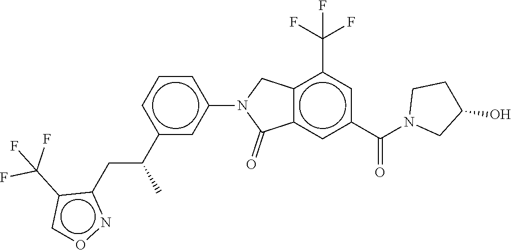 |
| 485 | 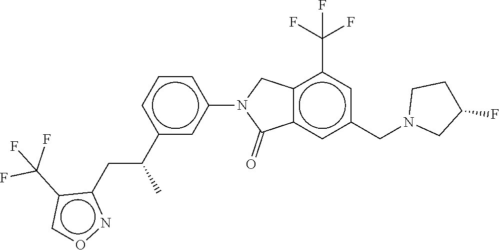 |
| 486a | 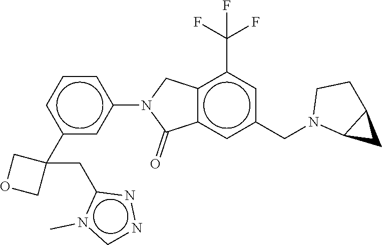 |
| 486b | 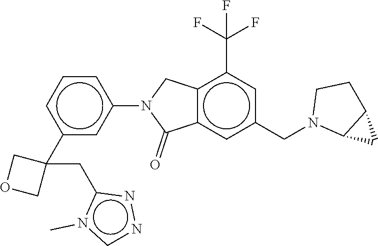 |
| 487 | 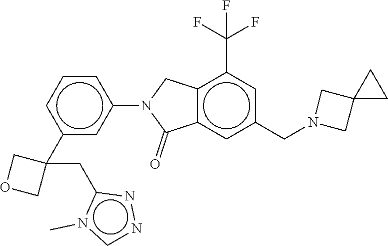 |
| 488 | 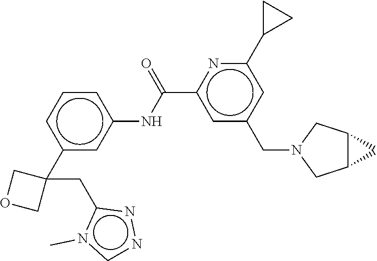 |
| 489 | 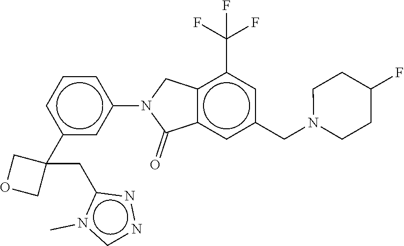 |
| 490 | 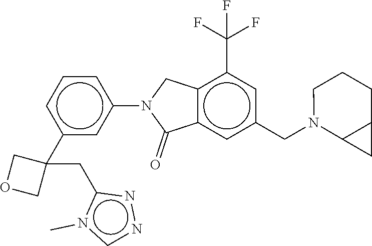 |
| 491 | 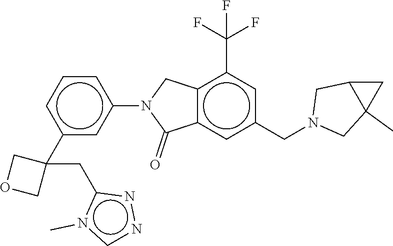 |
| 492 | 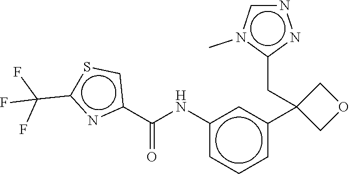 |
| 493 | 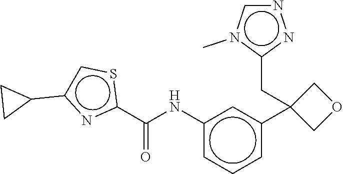 |
| 494 | 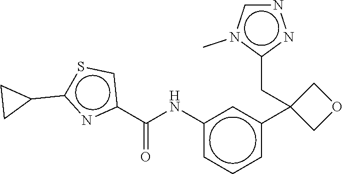 |
| 495 | 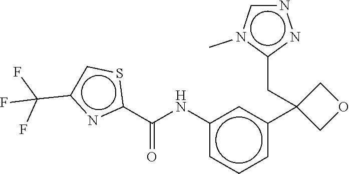 |
| 496 | 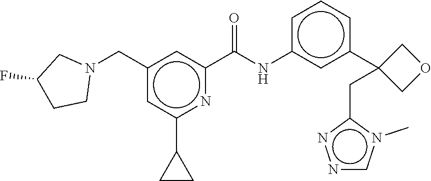 |
| 497 | 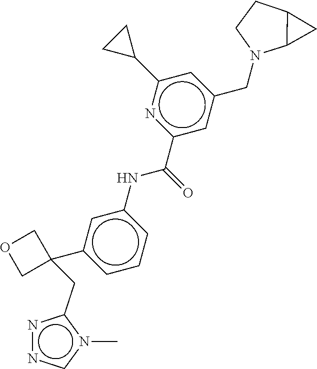 |
| 498 | 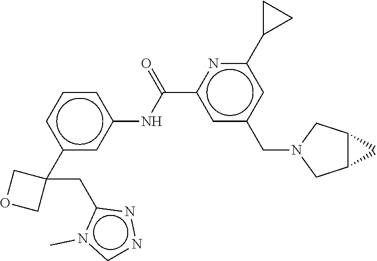 |
| 499 | 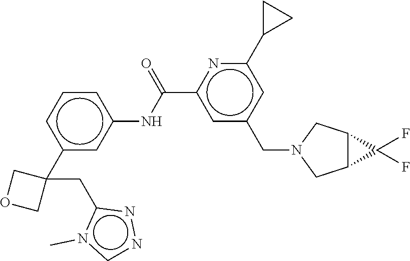 |
| 500 | 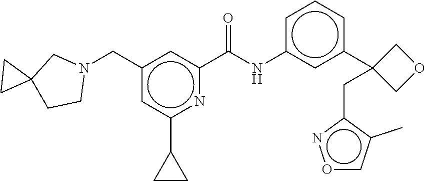 |
| 501 | 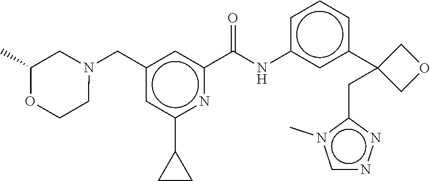 |
| 502 | 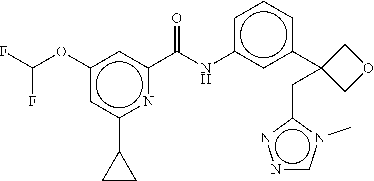 |
| 503 | 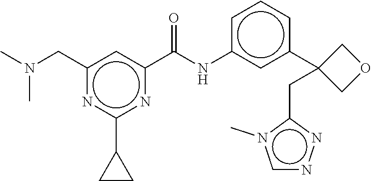 |
| 504 | 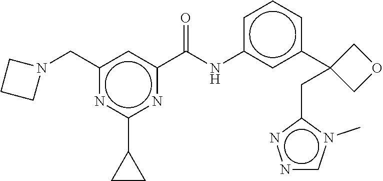 |
| 505 | 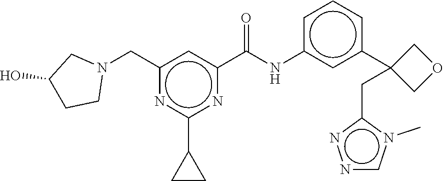 |
| 506 | 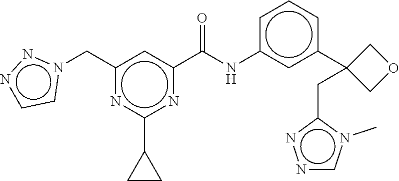 |
| 507 | 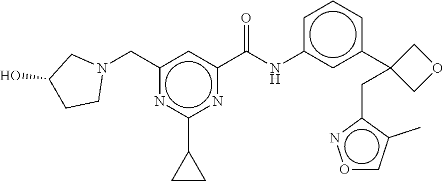 |
| 508 | 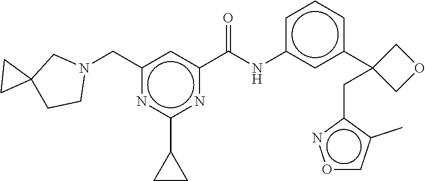 |
| 509 | 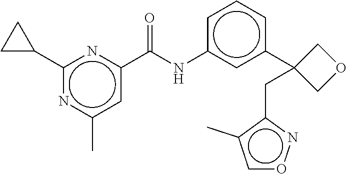 |
| 510 | 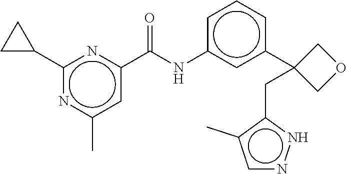 |
| 511 | 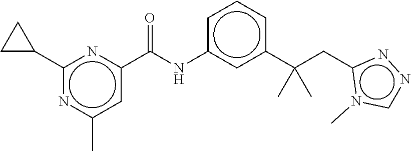 |
| 512 | 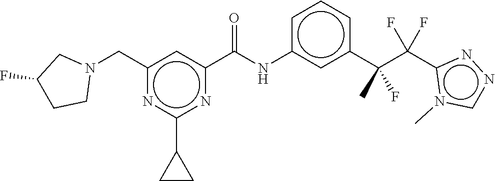 |
| 513 | 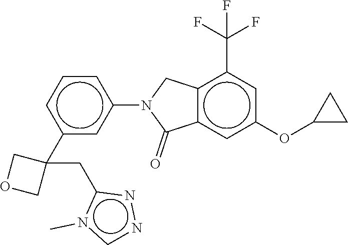 |
| 514 | 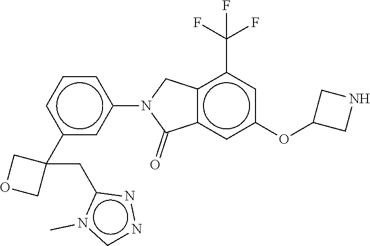 |
| 515 | 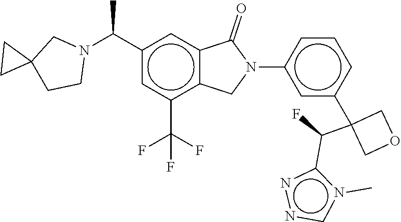 |
| 516 | 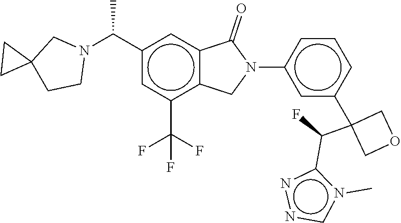 |
| 517a | 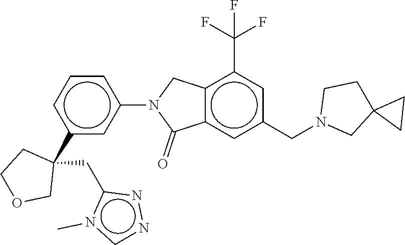 |
| 517b | 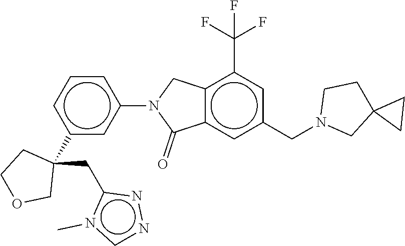 |
| 518 | 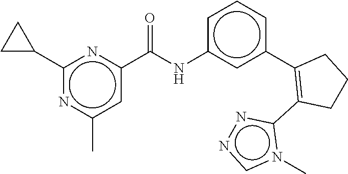 |
| 519 | 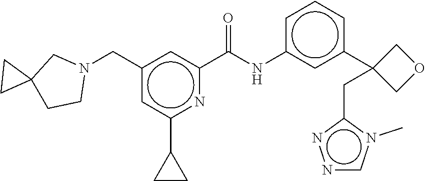 |
| 520 | 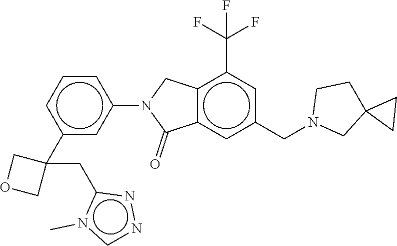 |
| 521 | 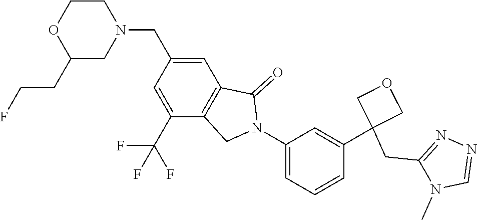 [SMILES: Cn1cnnc1CC1(c2cccc(N3Cc4c(cc(CN5CCOC(CCF)C5)cc4C(F)(F)F)C3=O)c2)COC1] |
| 522 | 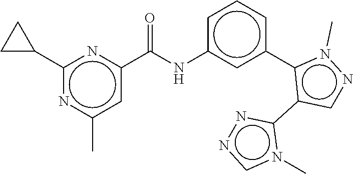 |
| 523 | 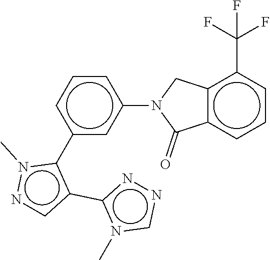 |
| 524 | 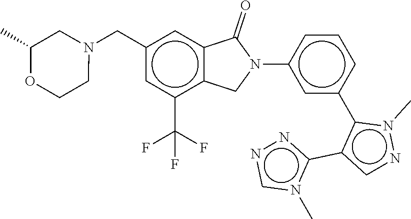 |
| 525 | 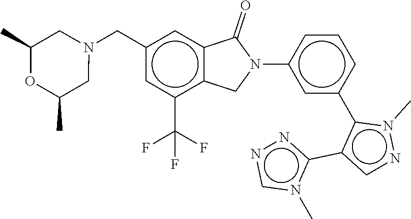 |
| 526 | 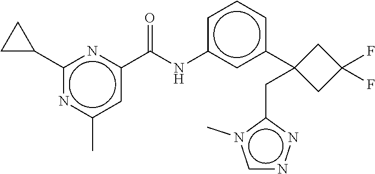 |
| 527 | 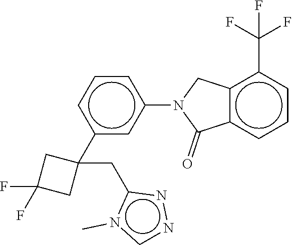 |
| 528 | 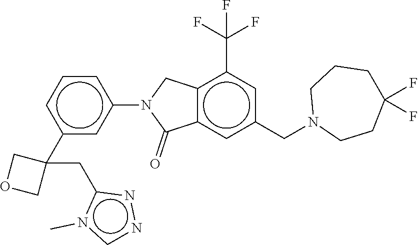 |
| 529 | 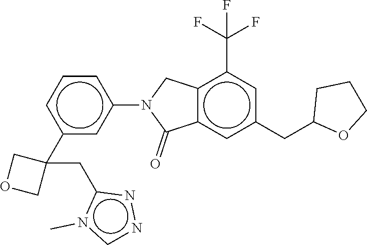 |
| 530 | 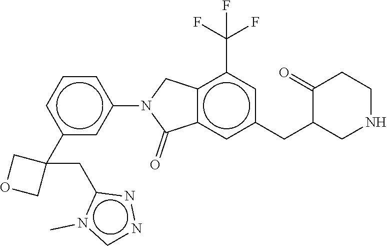 |
| 531 | 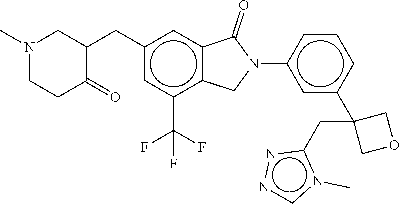 |
| 532 | 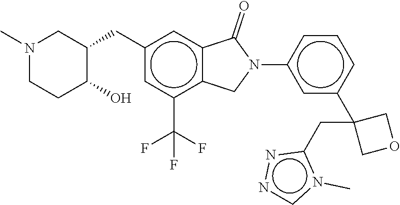 |
| 533 | 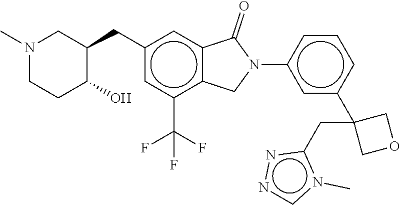 |
| 534a | 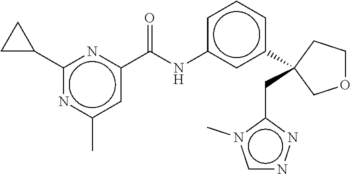 |
| 534b | 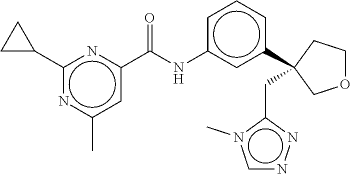 |
| 535 | 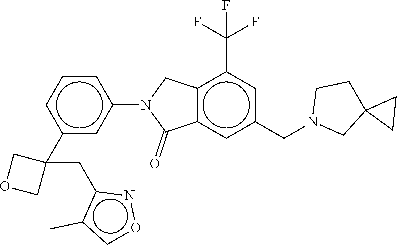 |
| 536 | 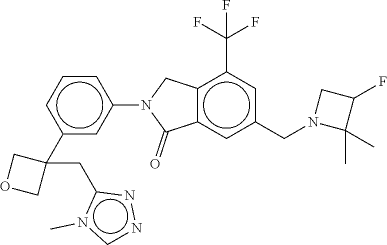 |
| 537 | 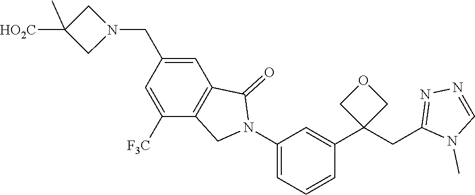 [SMILES: Cn1cnnc1CC1(c2cccc(N3Cc4c(cc(CN5CC(C)(C(=O)O)C5)cc4C(F)(F)F)C3=O)c2)COC1] |
| 538 | 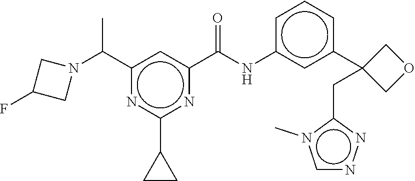 |
| 539 | 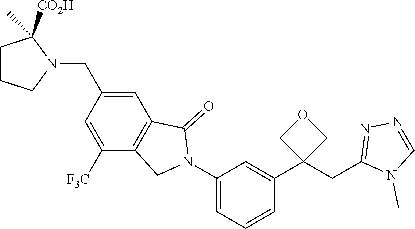 [SMILES: Cn1cnnc1CC1(c2cccc(N3Cc4c(cc(CN5CCC[C@@]5(C)C(=O)O)cc4C(F)(F)F)C3=O)c2)COC1] |
| 540 | 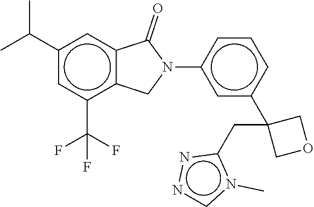 |
| 541a | 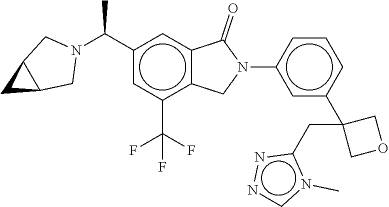 |
| 541b | 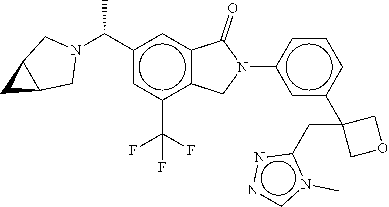 |
| 542 | 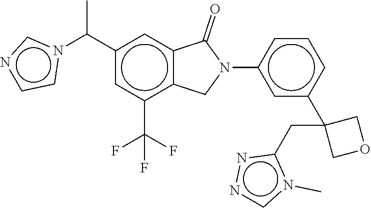 |
| 543a | 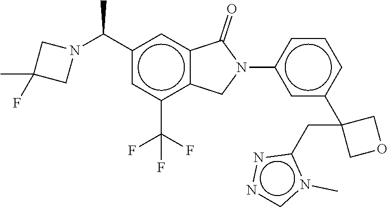 |
| 543b | 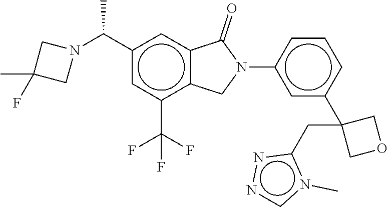 |
| 544 | 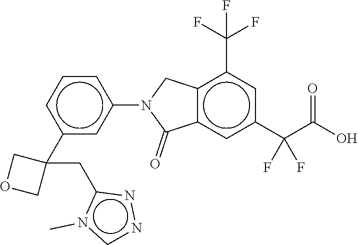 |
| 545 | 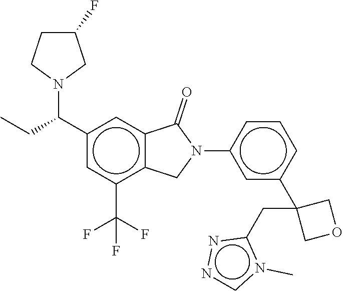 |
| 546 | 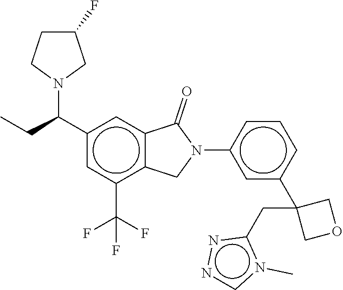 |
| 547 | 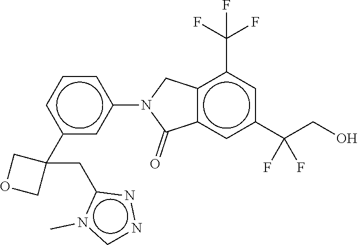 |
| 548 | 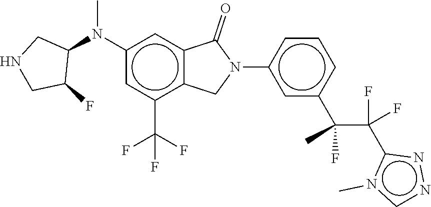 |
| 549 | 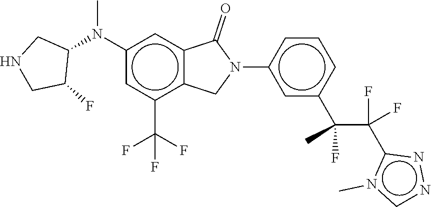 |
| 550 | 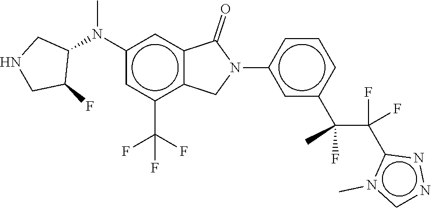 |
| 551 | 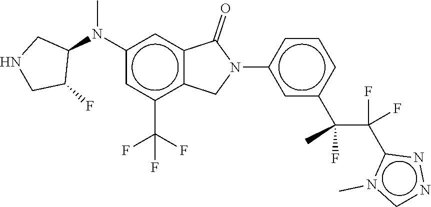 |
| 552 | 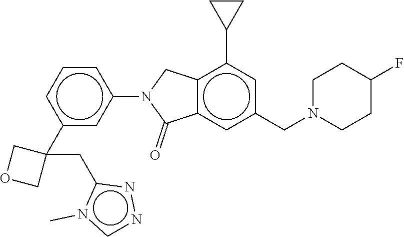 |
| 553a | 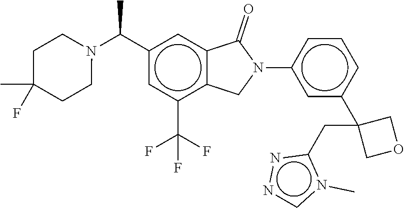 |
| 553b | 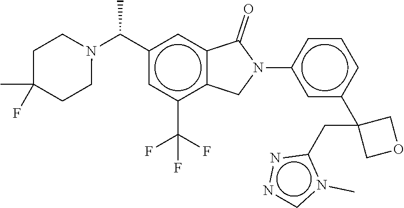 |
| 554 | 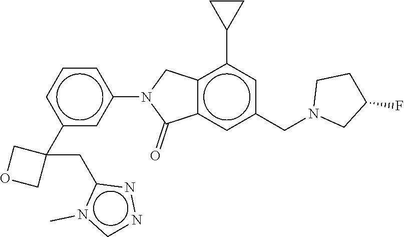 |
| 555 | 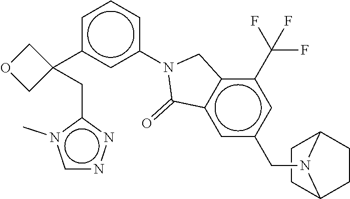 |
| 556 | 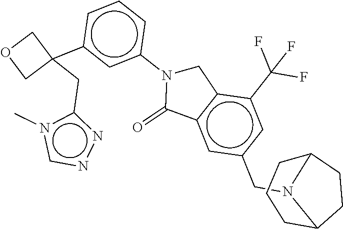 |
| 557 | 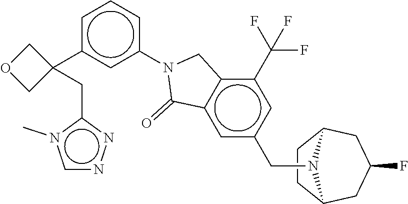 |
| 558a | 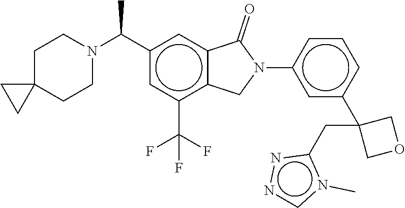 |
| 558b | 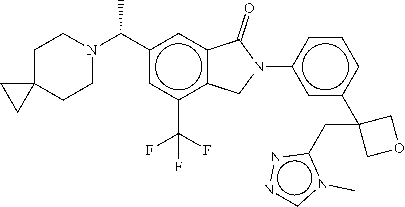 |
| 559 | 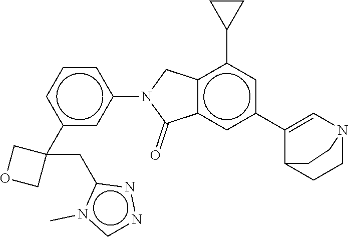 |
| 560 | 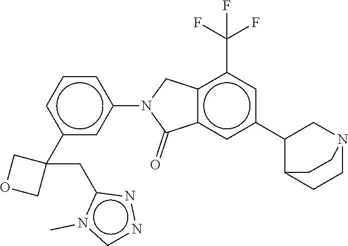 |
| 561 | 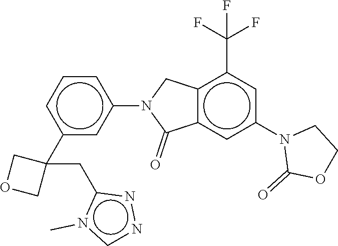 |
| 562 | 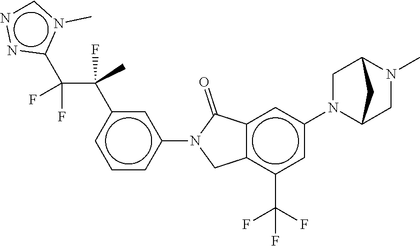 |
| 563 | 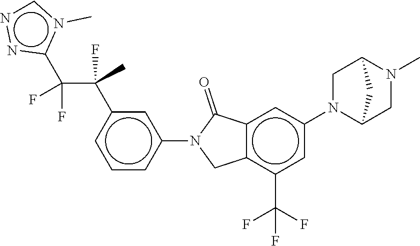 |
| 564 | 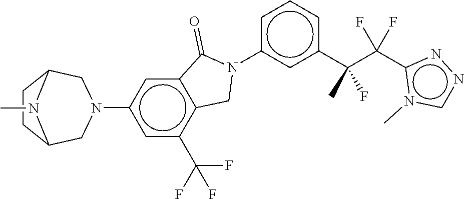 |
| 565 | 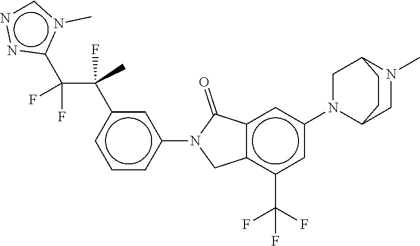 |
| 566 | 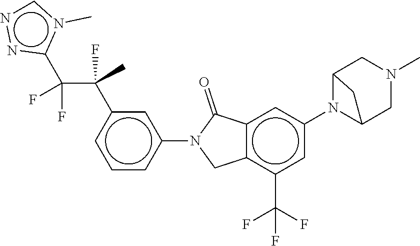 |
| 567 | 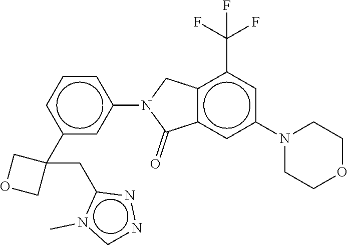 |
| 568 | 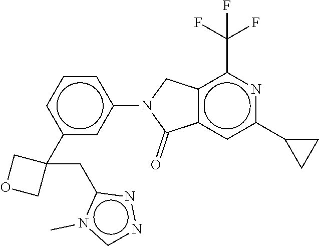 |
| 569 | 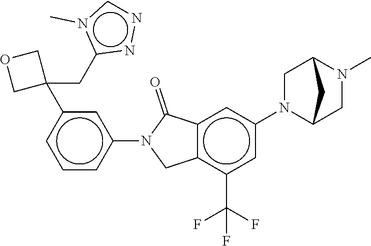 |
| 570 | 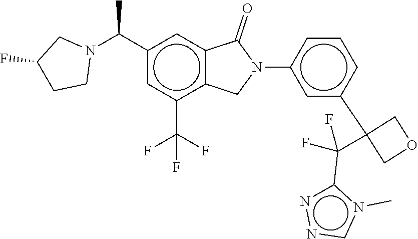 |
| 571 | 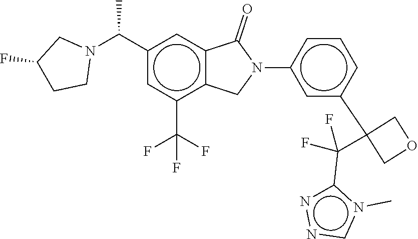 |
| 572 | 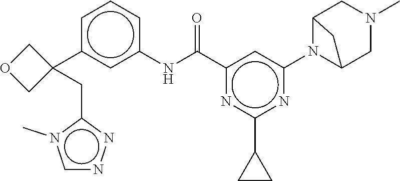 |
| 573 | 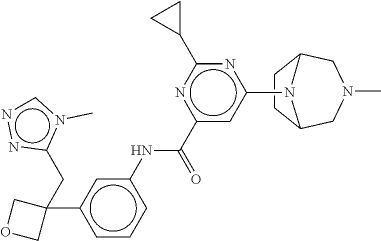 |
| 574 | 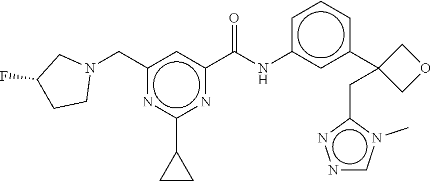 |
| 575 | 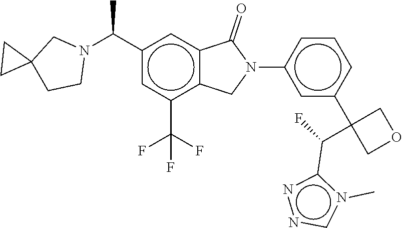 |
| 576 | 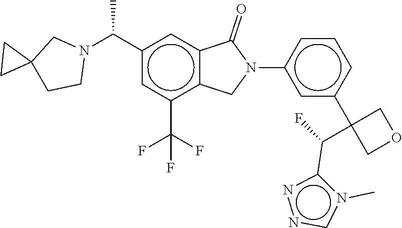 |
| 577 | 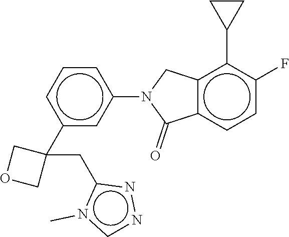 |
| 578 | 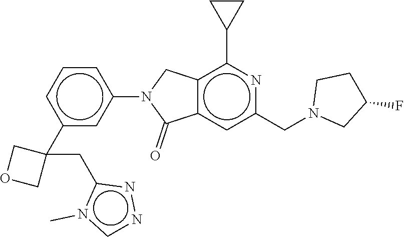 |
| 579 | 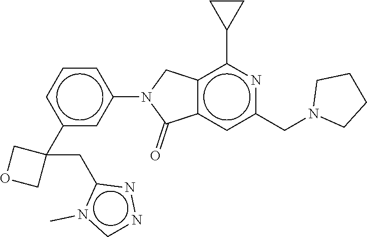 |
| 580 | 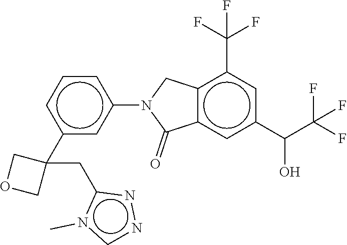 |
| 581 | 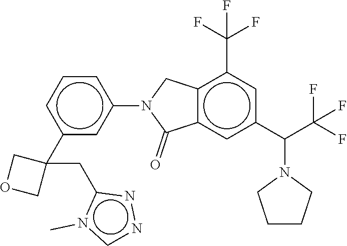 |
| 582 | 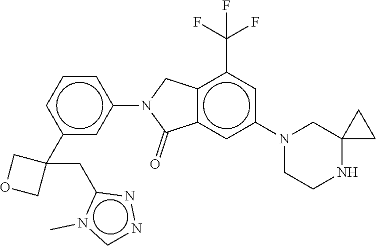 |
| 583 | 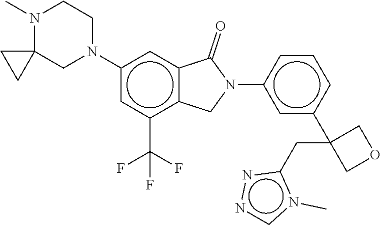 |
| 584 | 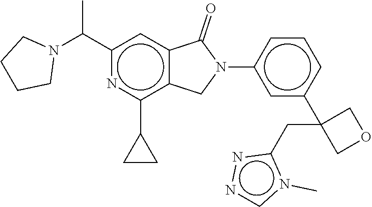 |
| 585 | 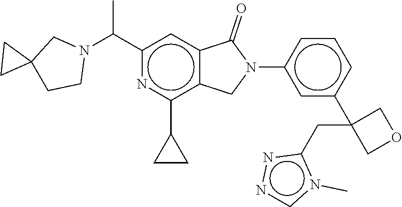 |
| 586 | 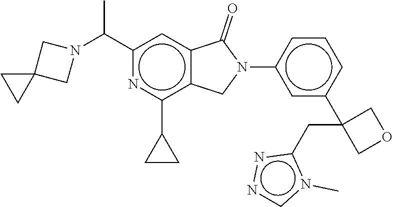 |
| 587 | 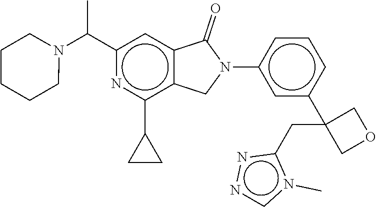 |
| 588 | 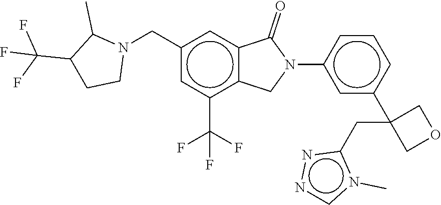 |
| 589 | 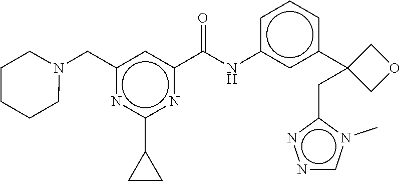 |
| 590 | 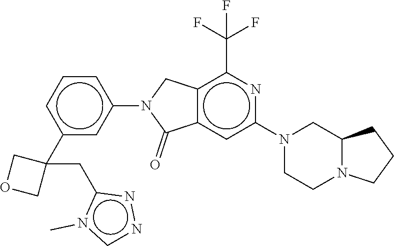 |
| 591 | 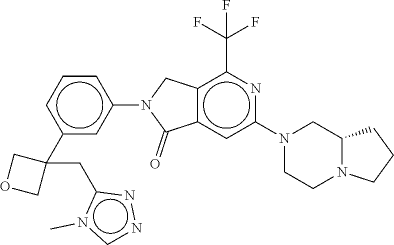 |
| 592a | 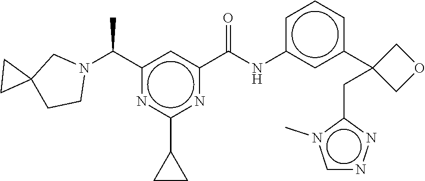 |
| 592b | 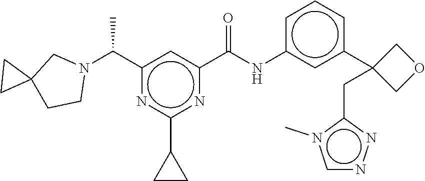 |
| 593 | 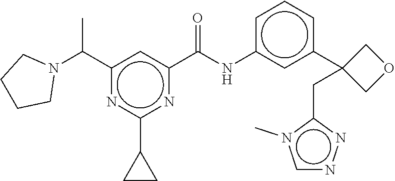 |
| 594 | 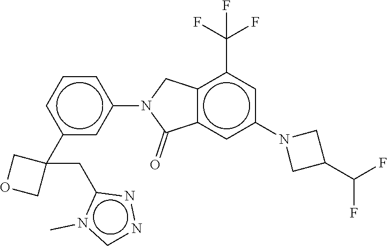 |
| 595 | 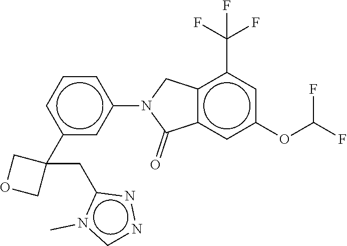 |
| 596 | 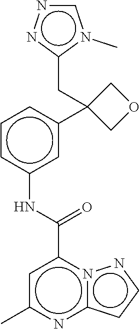 |
| 597 | 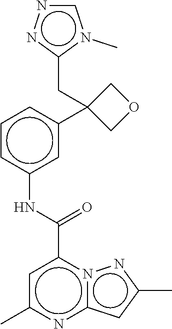 |
| 598 | 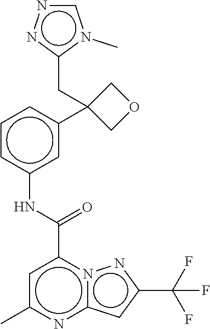 |
| 599 | 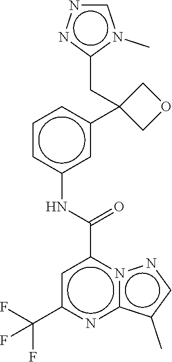 |
| 600 | 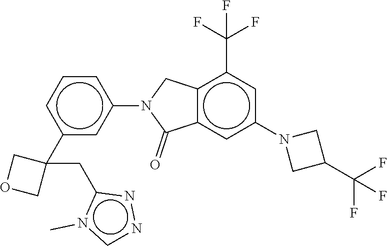 |
| 601 | 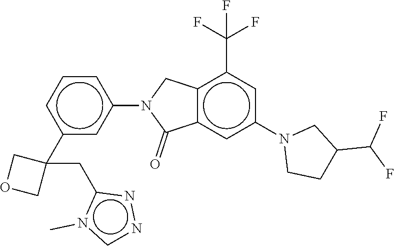 |
| 602 | 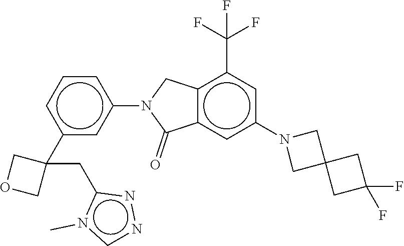 |
| 603 | 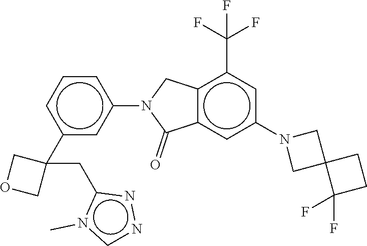 |
| 604 | 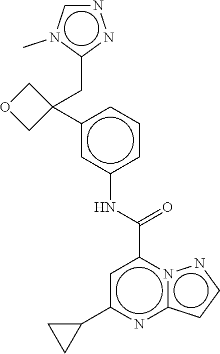 |
| 605 | 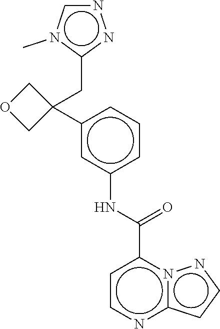 |
| 606 | 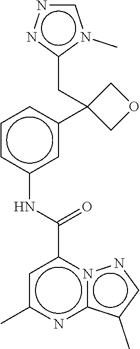 |
| 607 | 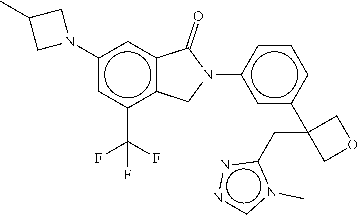 |
| 608 | 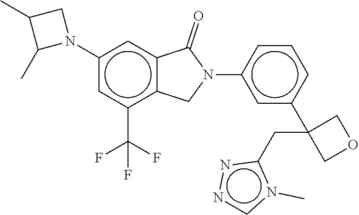 |
| 609 | 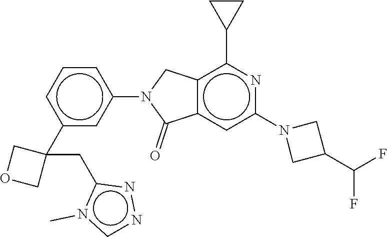 |
| 610 | 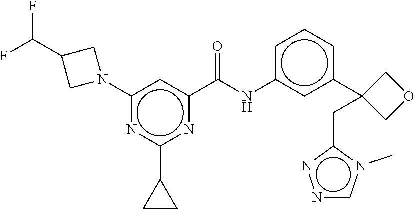 |
| 611 | 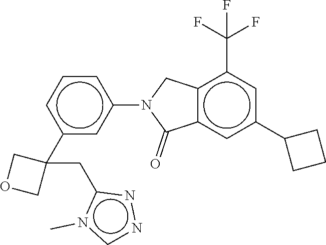 |
| 612 | 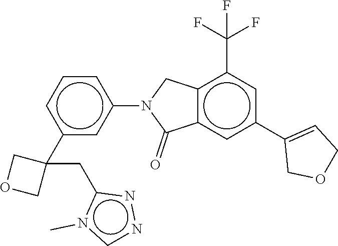 |
| 613 | 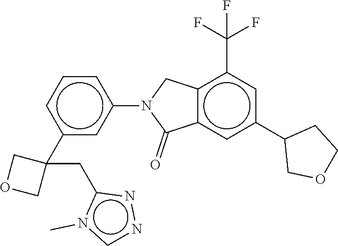 |
| 614 | 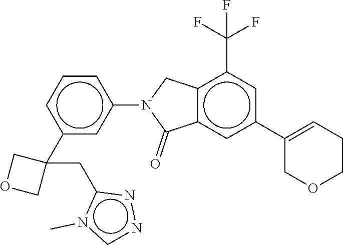 |
| 615 | 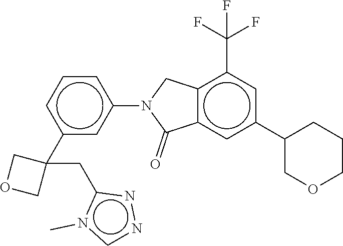 |
| 616 | 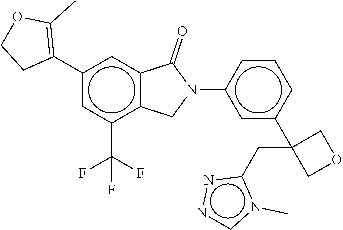 |
| 617 | 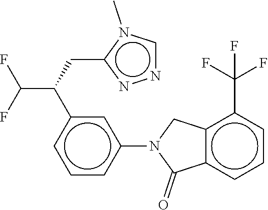 |
| 618 | 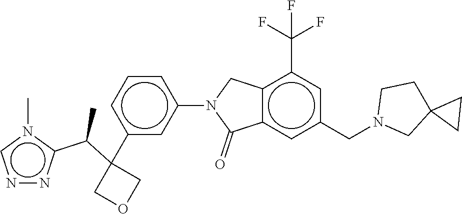 |
| 619 | 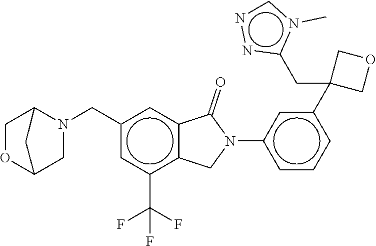 |
| 620 | 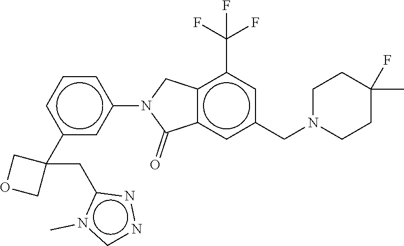 |
| 621 | 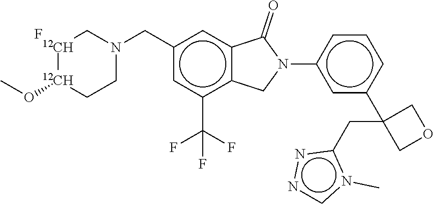 |
| 622a | 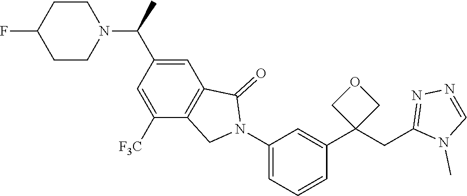 [SMILES: C[C@@H](c1cc2c(c(C(F)(F)F)c1)CN(c1cccc(C3(Cc4nncn4C)COC3)c1)C2=O)N1CCC(F)CC1] |
| 622b | 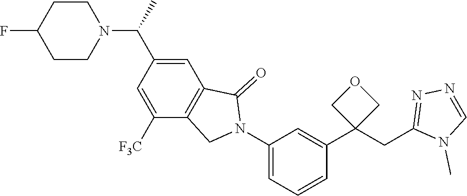 [SMILES: C[C@H](c1cc2c(c(C(F)(F)F)c1)CN(c1cccc(C3(Cc4nncn4C)COC3)c1)C2=O)N1CCC(F)CC1] |
| 623 | 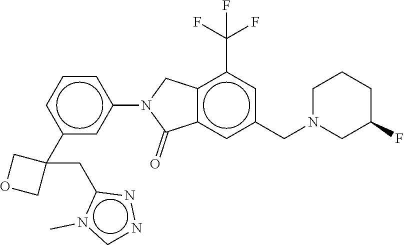 |
| 624 | 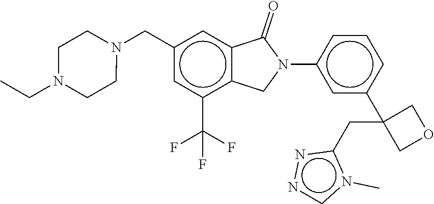 |
| 625 | 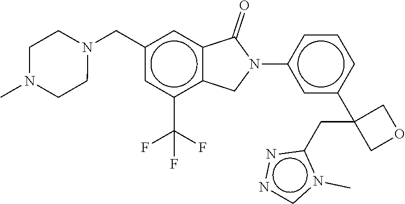 |
| 626a | 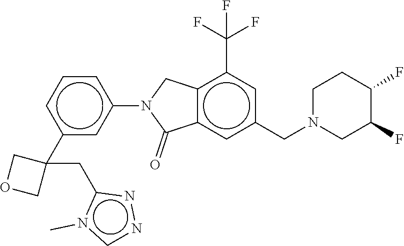 |
| 626b | 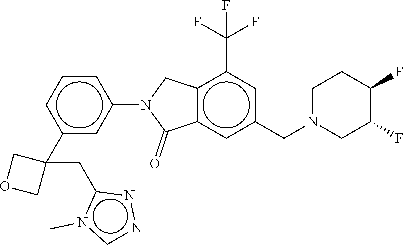 |
| 627 | 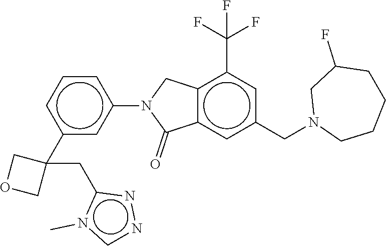 |
| 628 | 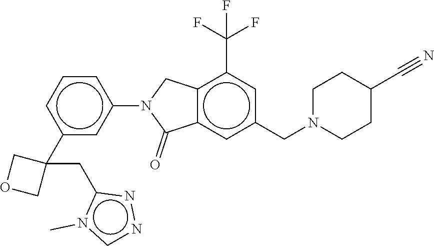 |
| 629 | 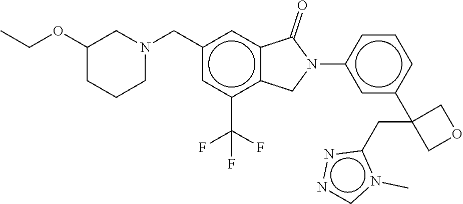 |
| 630 | 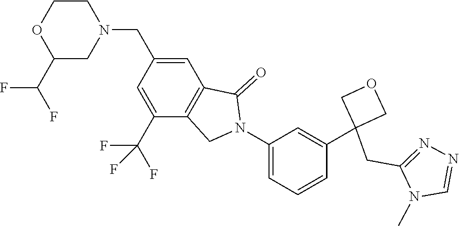 [SMILES: Cn1cnnc1CC1(c2cccc(N3Cc4c(cc(CN5CCOC(C(F)F)C5)cc4C(F)(F)F)C3=O)c2)COC1] |
| 631 | 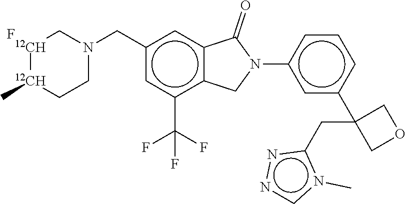 |
| 632 | 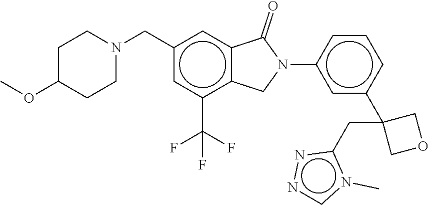 |
| 633 | 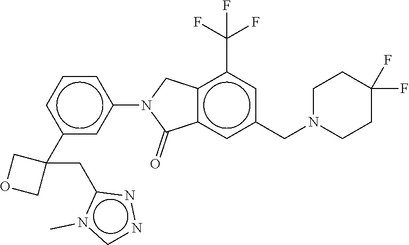 |
| 634 | 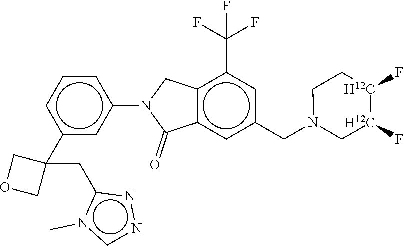 |
| 635 | 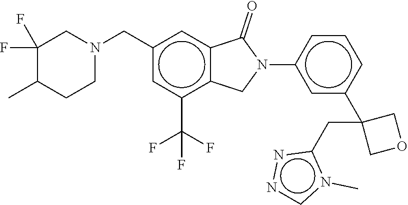 |
| 636 | 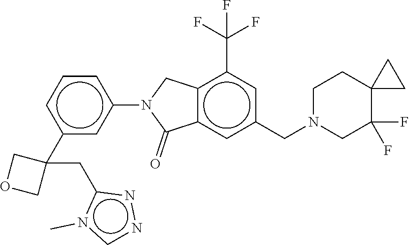 |
| 637 | 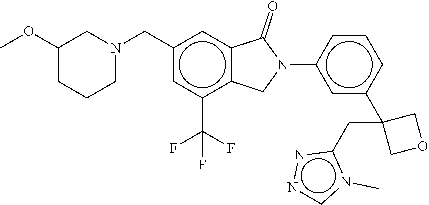 |
| 638 | 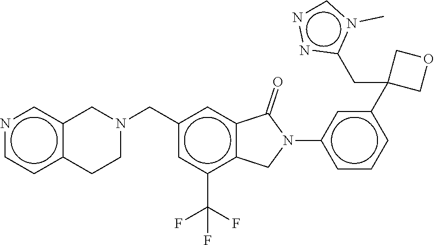 |
| 639a | 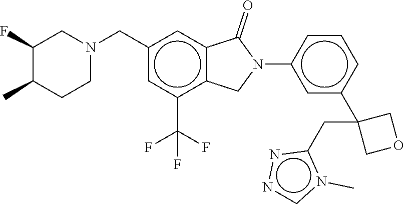 |
| 639b | 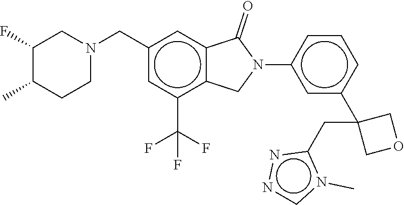 |
| 640 | 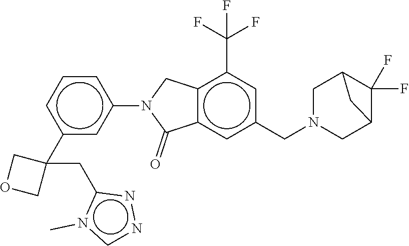 |
| 641 | 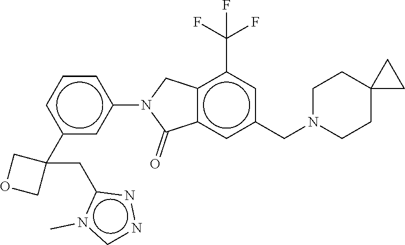 |
| 642 | 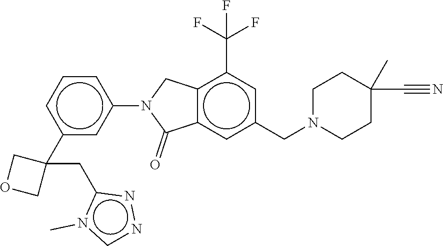 |
| 643 | 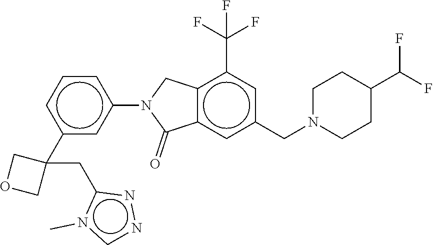 |
| 644a | 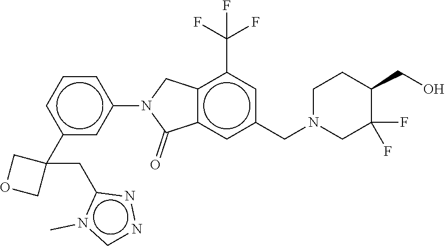 |
| 644b | 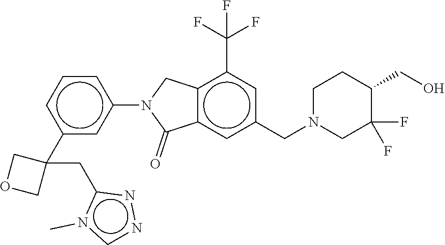 |
| 645 | 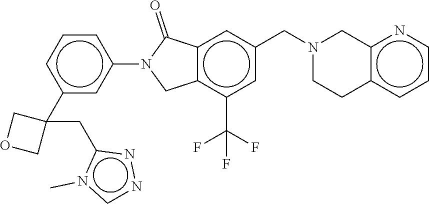 |
| 646 | 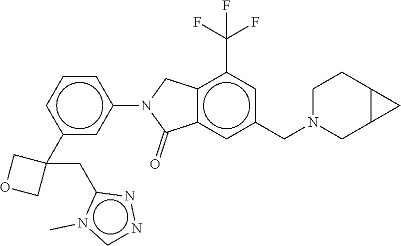 |
| 647 | 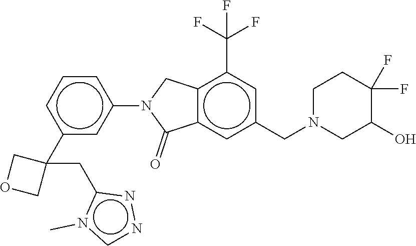 |
| 648 | 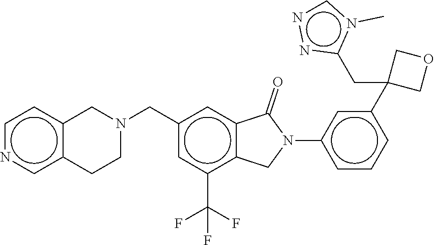 |
| 649 | 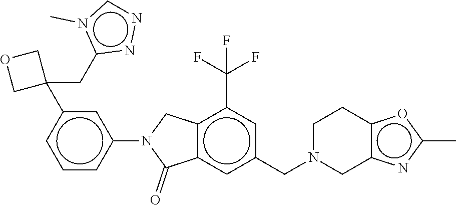 |
| 650 | 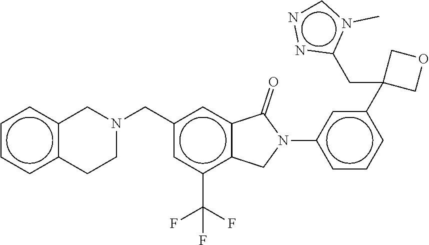 |
| 651 | 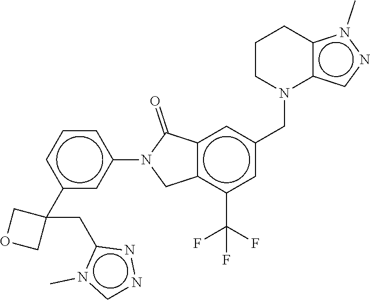 |
| 652 | 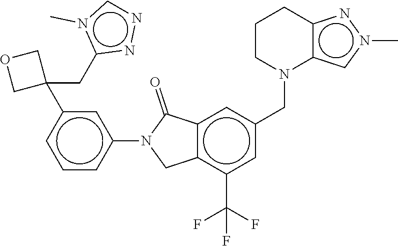 |
| 653 | 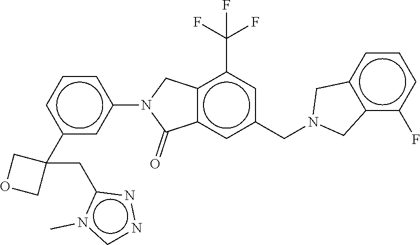 |
| 654 | 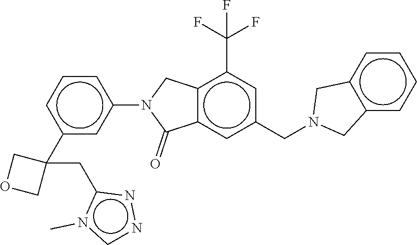 |
| 655 | 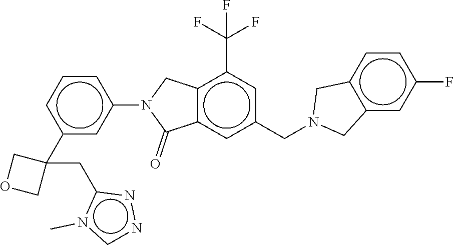 |
| 656 | 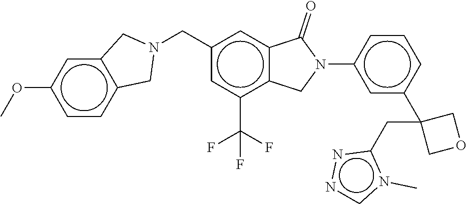 |
| 657 | 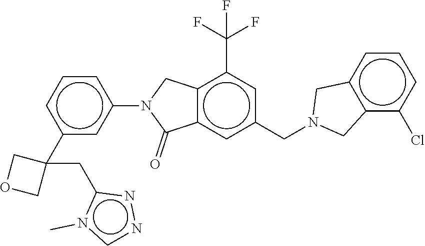 |
| 658 | 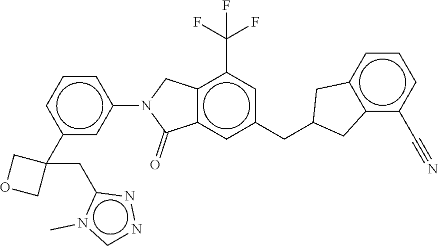 |
| 659 | 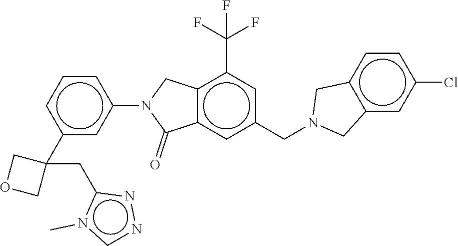 |
| 660 | 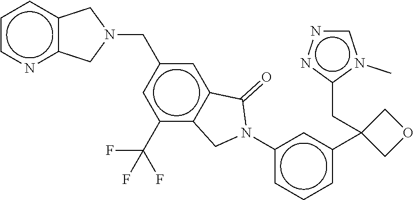 |
| 661 | 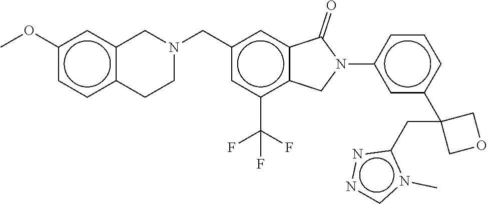 |
| 662a | 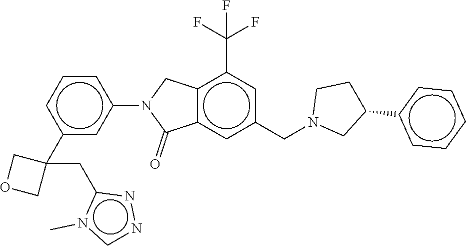 |
| 662b | 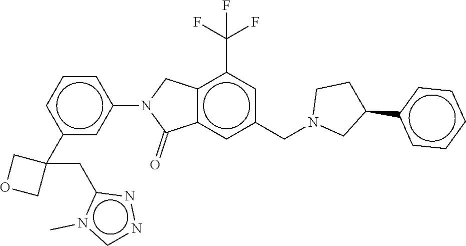 |
| 663a | 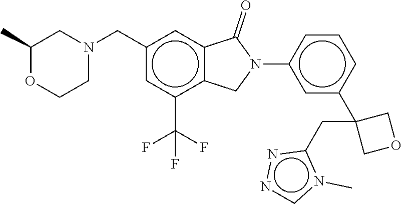 |
| 663b | 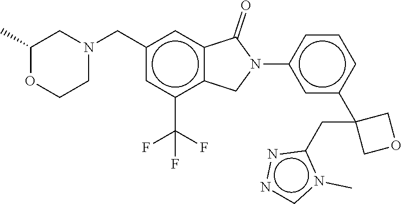 |
| 664 | 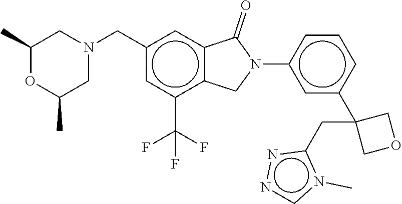 |
| 665 | 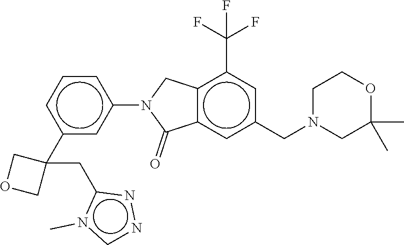 |
| 666 | 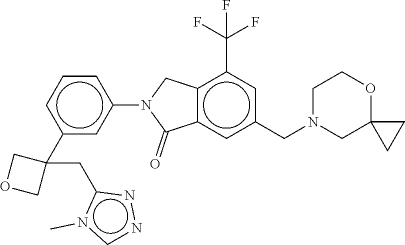 |
| 667 | 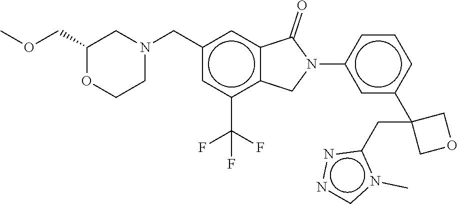 |
| 668a | 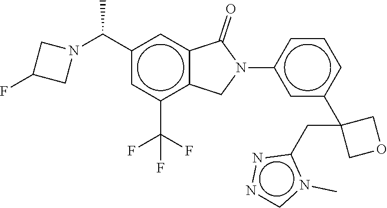 |
| 668b | 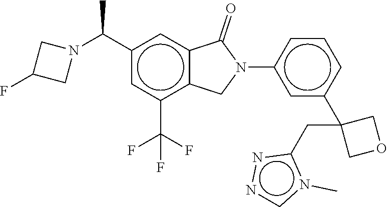 |
| 669a | 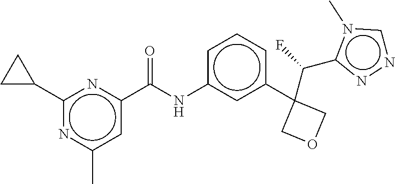 |
| 669b | 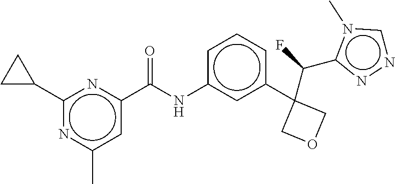 |
| 670a | 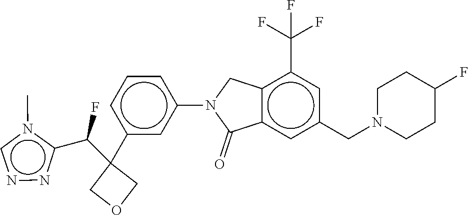 |
| 670b | 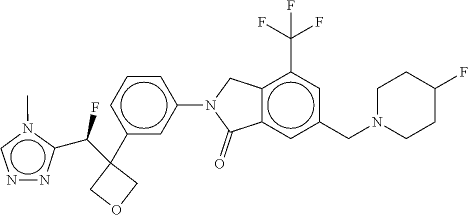 |
| 671a | 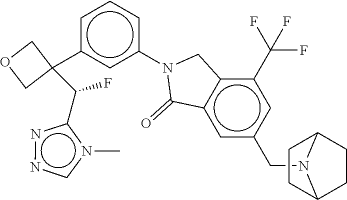 |
| 671b | 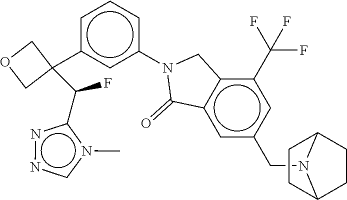 |
| 672a | 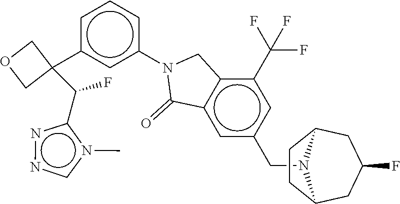 |
| 672b | 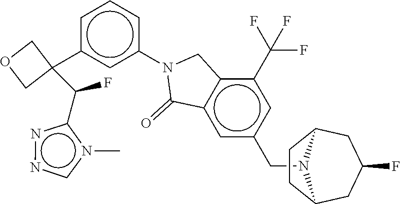 |
| 673 | 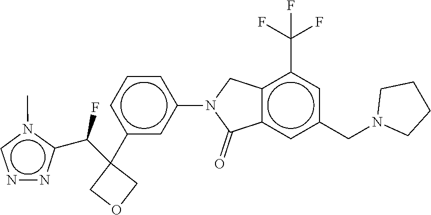 |
| 673b | 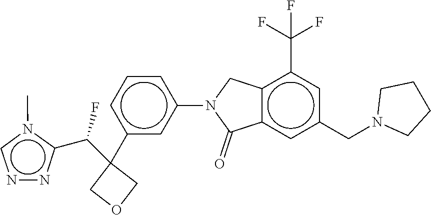 |
| 674a | 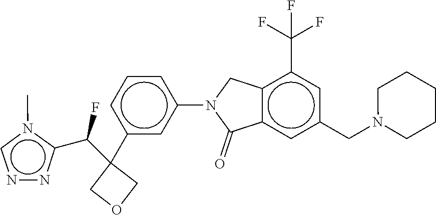 |
| 674b | 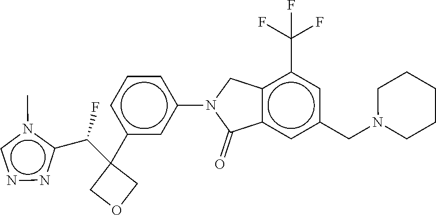 |
| 675a | 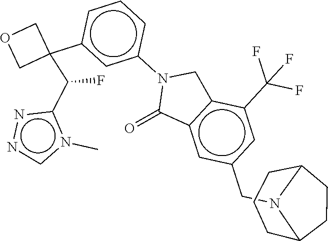 |
| 675b | 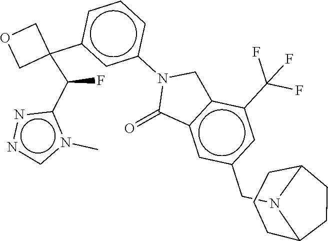 |
| 676 | 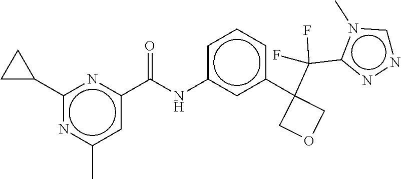 |
| 677 | 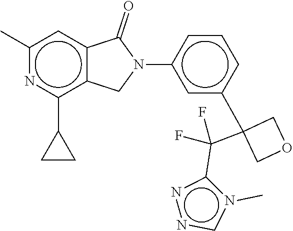 |
| 678 | 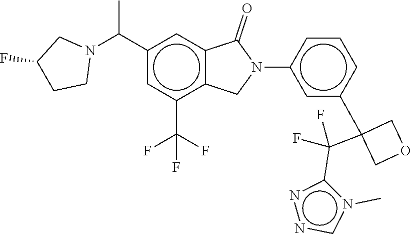 |
| 679 | 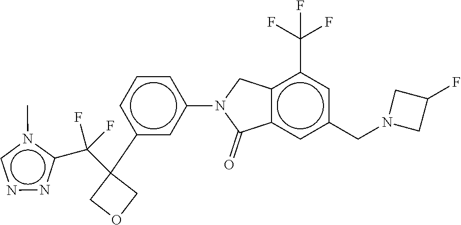 |
| 680 | 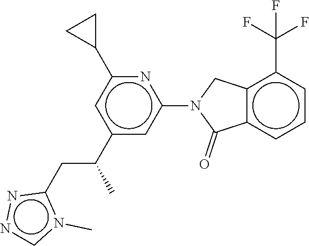 |
| 681 | 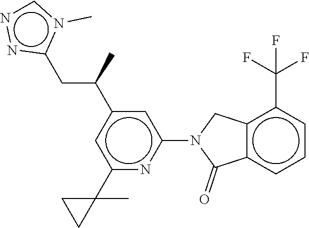 |
| 682 | 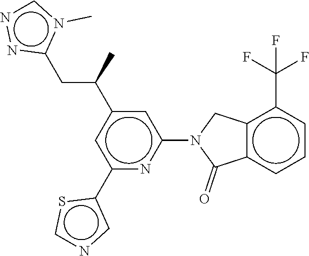 |
| 683 | 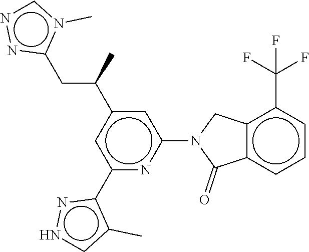 |
| 684 | 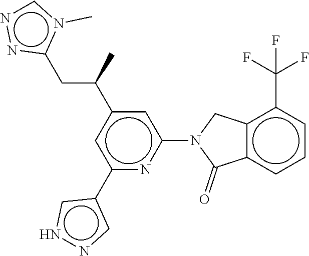 |
| 685 | 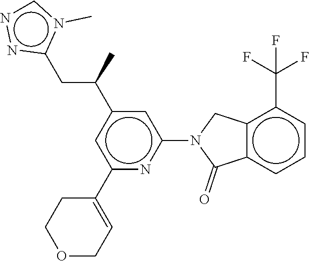 |
| 686 | 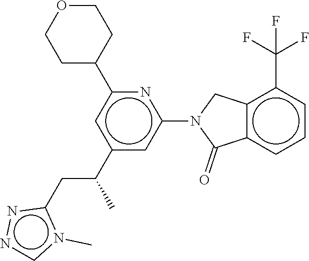 |
| 687 | 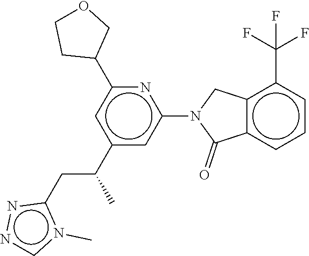 |
| 688 | 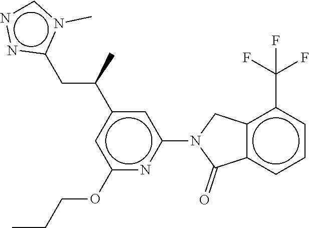 |
| 689a | 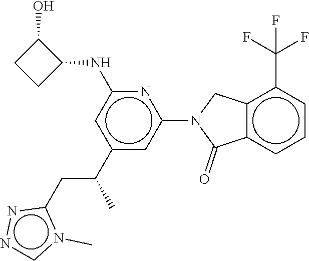 |
| 689b | 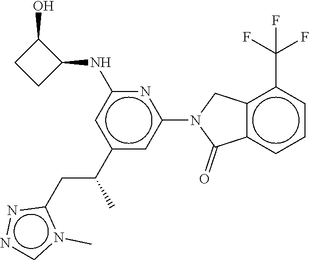 |
| 690 | 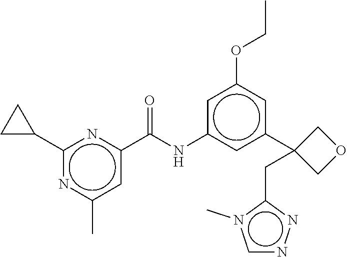 |
| 691a | 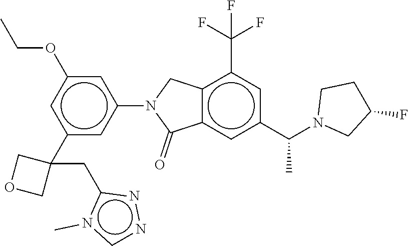 |
| 691b | 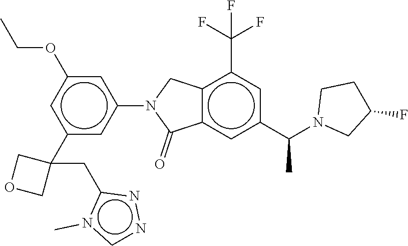 |
| 692 | 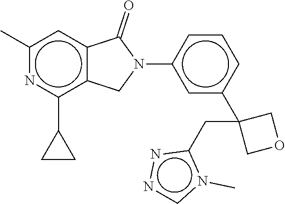 |
| 693 | 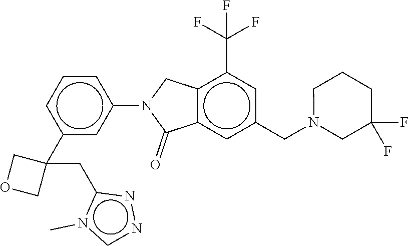 |
| 694 | 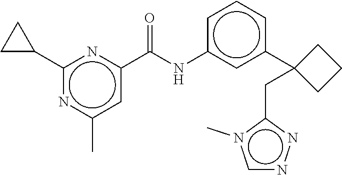 |
| 695 | 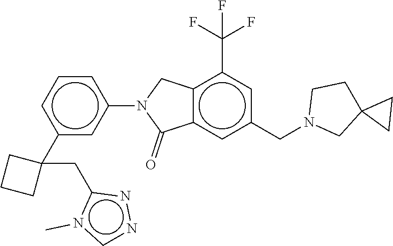 |
| 696 | 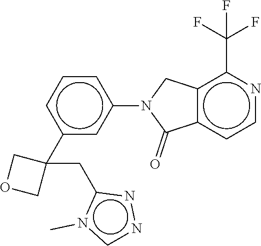 |
| 697 | 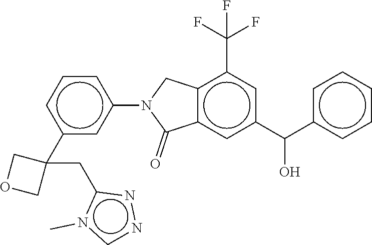 |
| 698 | 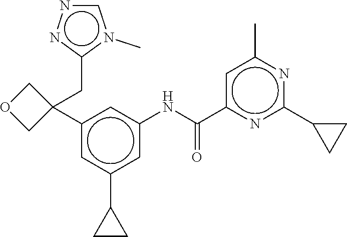 |
| 699 | 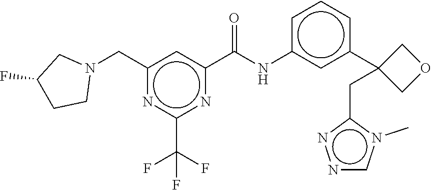 |
| 700 | 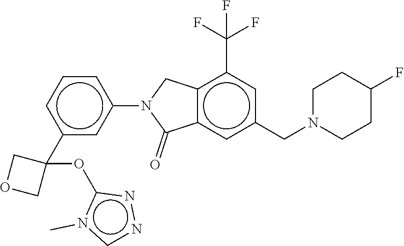 |
| 701 | 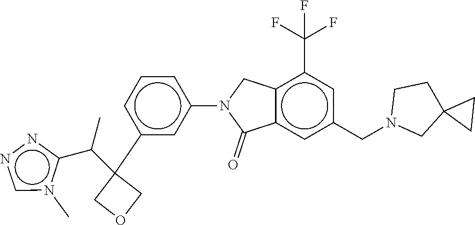 |
| 702 | 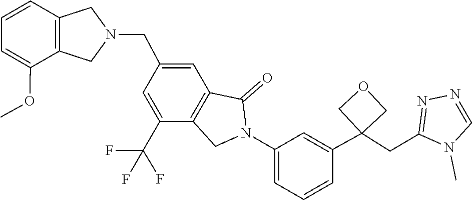 [SMILES: COc1cccc2c1CN(Cc1cc3c(c(C(F)(F)F)c1)CN(c1cccc(C4(Cc5nncn5C)COC4)c1)C3=O)C2] |
| 703 | 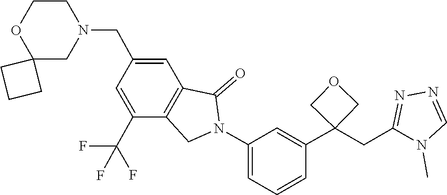 [SMILES: Cn1cnnc1CC1(c2cccc(N3Cc4c(cc(CN5CCOC6(CCC6)C5)cc4C(F)(F)F)C3=O)c2)COC1] |
| 704 | 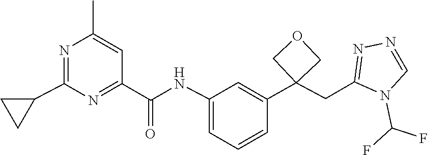 [SMILES: Cc1cc(C(=O)Nc2cccc(C3(Cc4nncn4C(F)F)COC3)c2)nc(C2CC2)n1] |
| 705 | 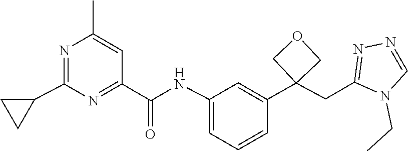 [SMILES: CCn1cnnc1CC1(c2cccc(NC(=O)c3cc(C)nc(C4CC4)n3)c2)COC1] |
| 706 | 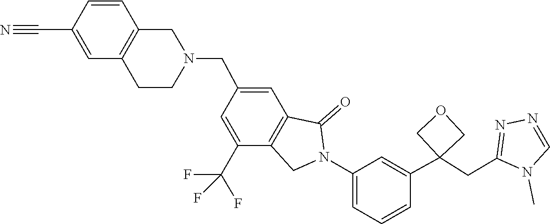 [SMILES: Cn1cnnc1CC1(c2cccc(N3Cc4c(cc(CN5CCc6cc(C#N)ccc6C5)cc4C(F)(F)F)C3=O)c2)COC1] |
| 707 | 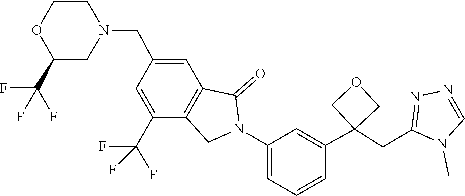 [SMILES: Cn1cnnc1CC1(c2cccc(N3Cc4c(cc(CN5CCO[C@H](C(F)(F)F)C5)cc4C(F)(F)F)C3=O)c2)COC1] |
| 708 | 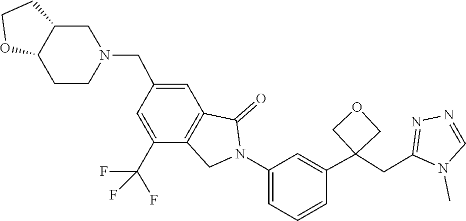 [SMILES: Cn1cnnc1CC1(c2cccc(N3Cc4c(cc(CN5CC[C@@H]6OCC[C@@H]6C5)cc4C(F)(F)F)C3=O)c2)COC1] |
| 709 | 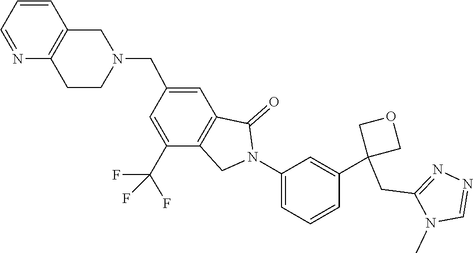 [SMILES: Cn1cnnc1CC1(c2cccc(N3Cc4c(cc(CN5CCc6ncccc6C5)cc4C(F)(F)F)C3=O)c2)COC1] |
| 710 | 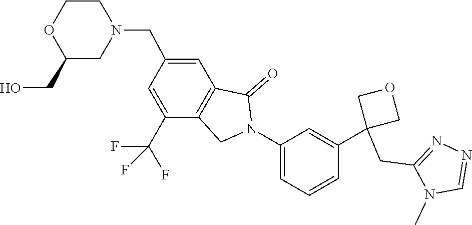 [SMILES: Cn1cnnc1CC1(c2cccc(N3Cc4c(cc(CN5CCO[C@H](CO)C5)cc4C(F)(F)F)C3=O)c2)COC1] |
| 711 | 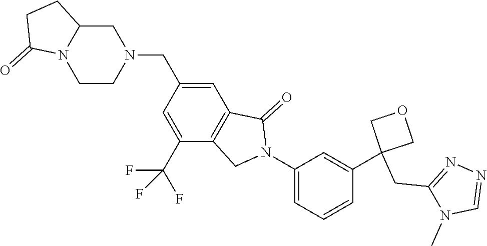 [SMILES: Cn1cnnc1CC1(c2cccc(N3Cc4c(cc(CN5CCN6C(=O)CCC6C5)cc4C(F)(F)F)C3=O)c2)COC1] |
| 712 | 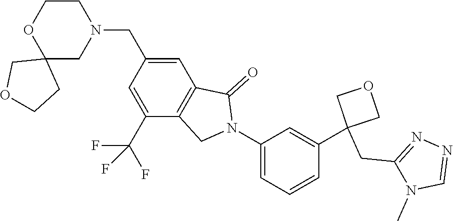 [SMILES: Cn1cnnc1CC1(c2cccc(N3Cc4c(cc(CN5CCOC6(CCOC6)C5)cc4C(F)(F)F)C3=O)c2)COC1] |
| 713 | 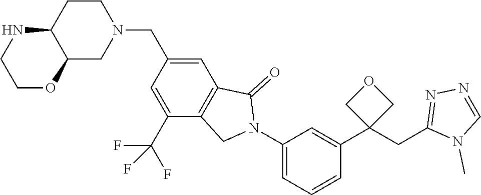 [SMILES: Cn1cnnc1CC1(c2cccc(N3Cc4c(cc(CN5CC[C@@H]6NCCO[C@@H]6C5)cc4C(F)(F)F)C3=O)c2)COC1] |
| 714 | 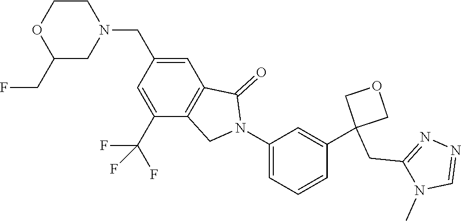 [SMILES: Cn1cnnc1CC1(c2cccc(N3Cc4c(cc(CN5CCOC(CF)C5)cc4C(F)(F)F)C3=O)c2)COC1] |
| 715 | 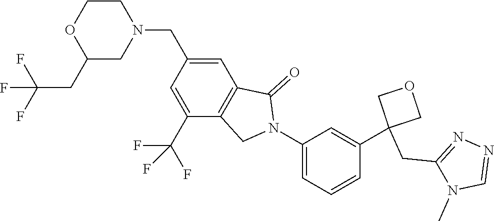 [SMILES: Cn1cnnc1CC1(c2cccc(N3Cc4c(cc(CN5CCOC(CC(F)(F)F)C5)cc4C(F)(F)F)C3=O)c2)COC1] |
| 716 | 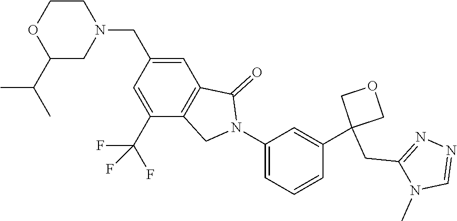 [SMILES: CC(C)C1CN(Cc2cc3c(c(C(F)(F)F)c2)CN(c2cccc(C4(Cc5nncn5C)COC4)c2)C3=O)CCO1] |
| 717 | 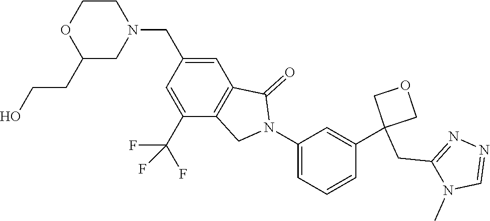 [SMILES: Cn1cnnc1CC1(c2cccc(N3Cc4c(cc(CN5CCOC(CCO)C5)cc4C(F)(F)F)C3=O)c2)COC1] |
| 718 | 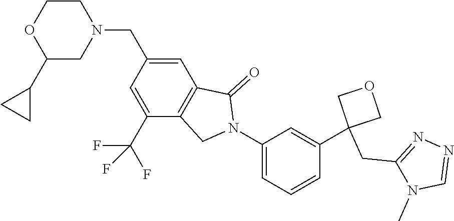 [SMILES: Cn1cnnc1CC1(c2cccc(N3Cc4c(cc(CN5CCOC(C6CC6)C5)cc4C(F)(F)F)C3=O)c2)COC1] |
| 719 | 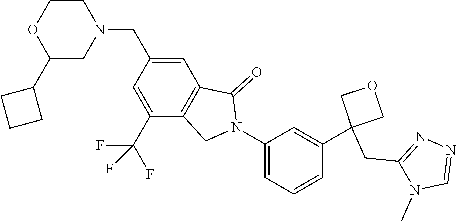 [SMILES: Cn1cnnc1CC1(c2cccc(N3Cc4c(cc(CN5CCOC(C6CCC6)C5)cc4C(F)(F)F)C3=O)c2)COC1] |

The schemes below describe methods of synthesizing the compounds disclosed herein, where the rings represented by Ring A, Ring C, and Ring B are as described in the specification and claims herein. Thus, for example, Ring C in the schemes below can be 1,3-substituted phenyl, 2,4-substituted pyridyl (i.e., the pyridine nitrogen is ortho to the branch of the molecule having Ring A, while the pyridine nitrogen is para to the branch of the molecule having Ring B), 3,5-substituted pyridyl, or iso-2,4-substituted pyridyl (i.e., the pyridine nitrogen is ortho to the branch of the molecule having Ring B, while the pyridine nitrogen is para to the branch of the molecule having Ring A). Similarly, Ring A in the schemes below can be any of the rings described in the specification and claims herein for Ring A, for example, including, but not limited to, a phenyl ring or a pyridazinyl ring; and Ring C in the schemes below can be any of the rings described in the specification and claims herein for Ring C, for example, including, but not limited to, triazolyl. One of skill in the art will appreciate that terms such as “a heterocyclic ring,” “a heteroaryl ring,” and the like can refer to a single ring or multiple condensed rings, unless otherwise indicated or clear from context.

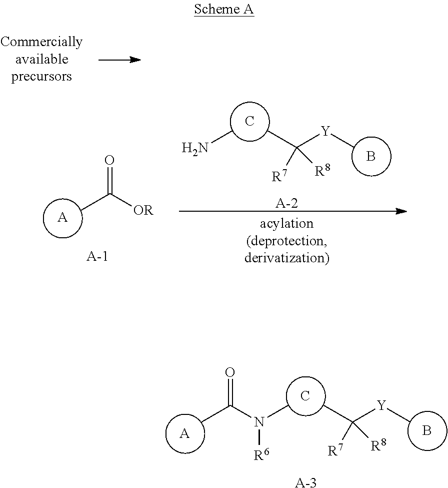

Compounds of the general formula A-3 can be readily synthesized from acids A-1 and anilines A-2 using standard amide bond formation conditions in standard solvents such as dimethyl formamide or methylene chloride. If acids A-1 or the anilines A-2 are derivatized by protected functional groups, these may be removed and subsequently derivatized as appropriate.

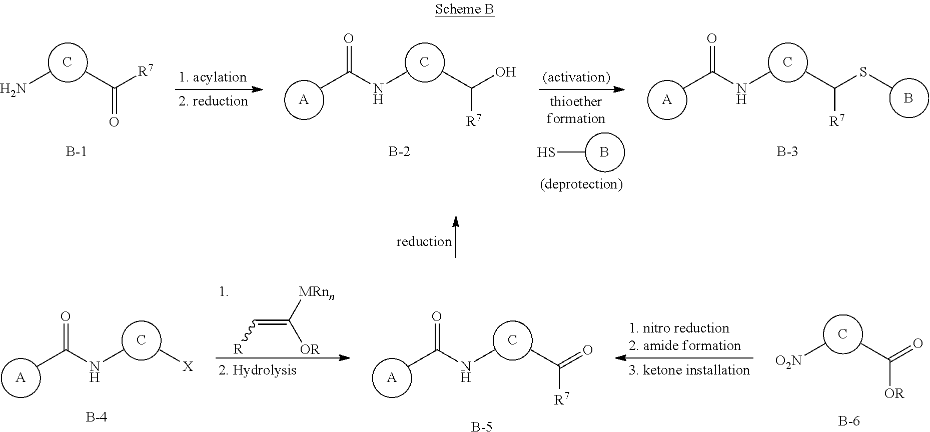

Scheme B outlines an alternate approach to synthesizing amides B-3. Ketone substituted amines B-1 may be acylated with acids A-1 under standard amide bond formation conditions (e.g., active ester formation, carbodiimide condensation) and reduced to yield the corresponding alcohols B-2. The secondary alcohols can be converted to thioethers B-3 either directly under conditions such as the Mitsunobu reaction or following activation of the alcohols and substitution reactions using thiol substituted heterocycles.

Intermediates B-2 may be obtained by reduction of the corresponding ketones B-5 that are accessible from aryl halides B-4 following metallated enolate cross-coupling reactions and subsequent hydrolysis of the enol ethers. Ester containing nitroarene B-6 can be reduced to the amino arenes which can be acylated with acids A-1. The intermediate esters can lead to ketones B-5 as well under standard conditions, for example using Weinreb amide chemistries.

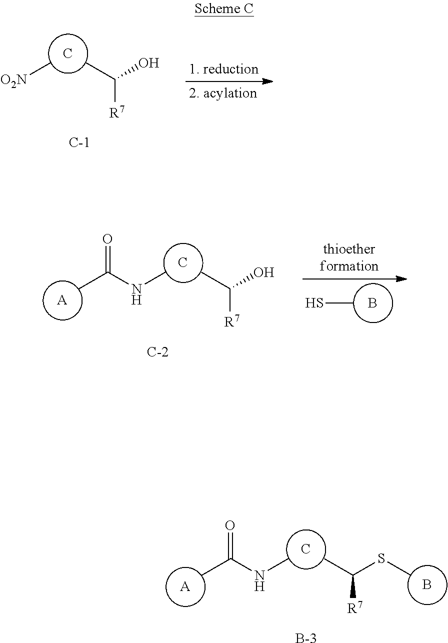

Another approach to amides B-3 is shown in scheme C. Enantioenriched nitro aryl alcohols C-1 may be reduced to the corresponding amines that can be acylated with acids A-1 to the corresponding amides C-2. Thioether formation can be carried under conditions such as the Mitsunobu reaction to yield amides B-3.

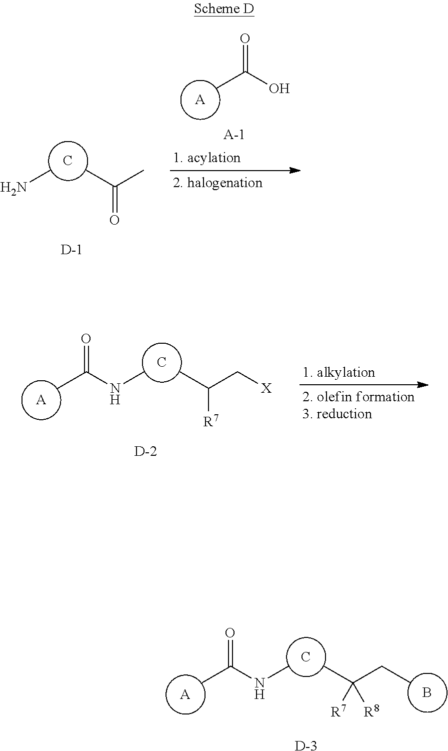

An approach to methylene-linked amides is shown in Scheme D. A 3-acetyl substituted amino aryl compound D-1 can be acylated with aryl carboxylic acids A-1 under standard conditions. The obtained methyl ketones can be halogenated under standard conditions to provide halomethyl ketones D-2. Subsequent displacement reactions on the halomethyl ketone using suitable heterocycles such as pyrazoles, triazoles, imidazole, tetrazoles, etc., followed by conversion of the ketone to an olefin (using, e.g., the Wittig reaction; see Example 216, step 4) and its subsequent reduction (e.g., by hydrogenation over Pd; see Example 216, step 5) leads to the formation of amides D-3.

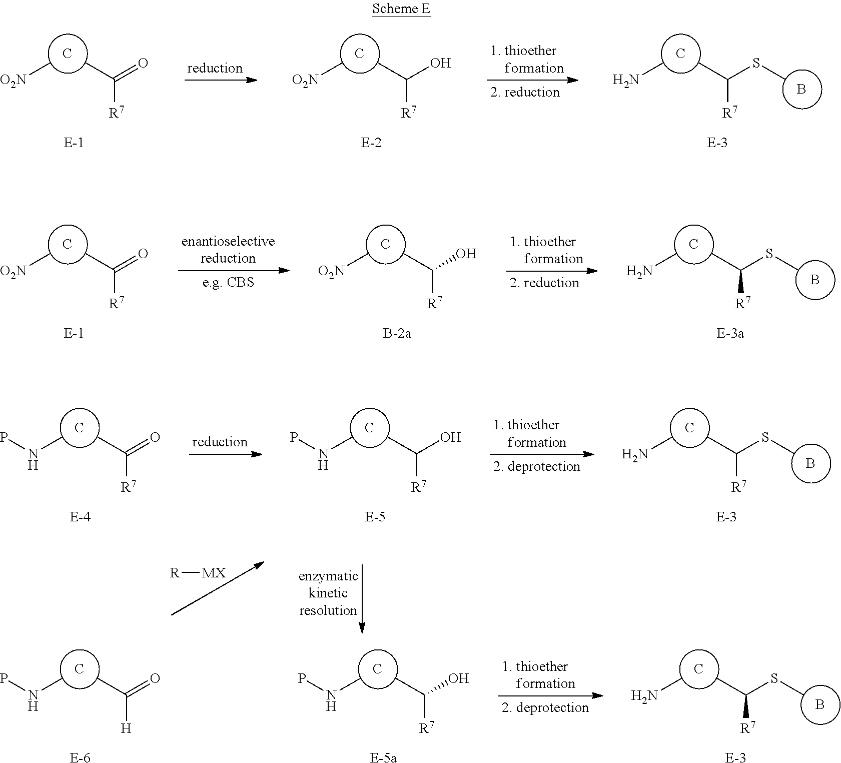

Thioether containing amines E-3 may be synthesized as depicted in scheme E. Nitro-aryl ketones E-1 can be reduced to the corresponding alcohols E-2 that can be utilized to prepare thioethers by reaction with thiol-containing heterocycles under thioether formation conditions such as the Mitsunobu reaction. The amines E-3 can be obtained following the reduction of the nitro group under standard conditions. In a related manner, nitro-aryl ketones E-1 can be reduced in an enantioenriched fashion using common methodology such as the Corey-Bakshi-Shibata (CBS) reagent. The enantioenriched alcohols E-2a may be elaborated in a similar manner to the corresponding thioethers E-3a using the same conditions as for amines B-3.

Appropriately protected amino arenes substituted with an acyl group (E-4) can be reduced under standard conditions to the corresponding alcohols E-5 followed by thioether formation under for example Mitsunobu conditions using appropriately functionalized heterocycles. The protecting groups (P) can be removed to yield the amino arene intermediates E-3. Intermediates E-5 also can be subjected to enzymatic kinetic resolution to provide enantioenriched alcohols E-5a that can be converted to the corresponding thioethers and subjected to standard deprotection conditions leading to the formation of enantioenriched amino arenes E-3a. Intermediates E-5 also can be synthesized from aldehydes E-6 by addition of an organometallic reagent such as a Grignard or organo-lithium reagent.

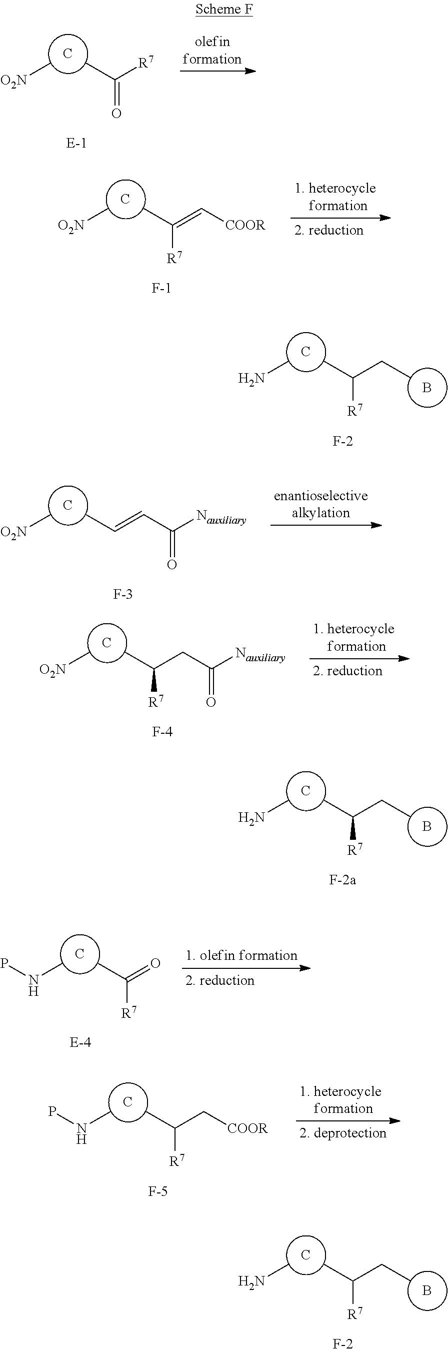

Methylene linked anilines such as F-2 can be synthesized as outlined in scheme F. Ketone E-1 can be elaborated to the corresponding olefin by using commonly used reagents such as a phosphonium ylide (such as in the Wittig reaction). Esters F-1 may be used as functional groups suitable for conversion to heterocycles under standard conditions such as depicted in schemes U-AC. Amino arenes F-2 then can be formed by subsequent reduction of the nitro group under standard conditions.

Cinnamic acids functionalized with chiral auxiliaries (F-3) may be used to synthesize enantioenriched intermediates F-4. Conversion of the amides to heterocyclic derivatives under standard conditions as depicted in schemes U-AC followed by nitro-reduction can lead to enantioenriched amines F-2a.

Appropriately protected amino arenes E-4 also can be elaborated to esters F-5 using reagents such as the phosphonium ylides (for example Wittig reaction) followed by standard olefin reducing conditions. Esters F-5 can be used to form heterocycles as depicted in schemes U-AC followed by nitro reduction to yield amino arene intermediates F-2.

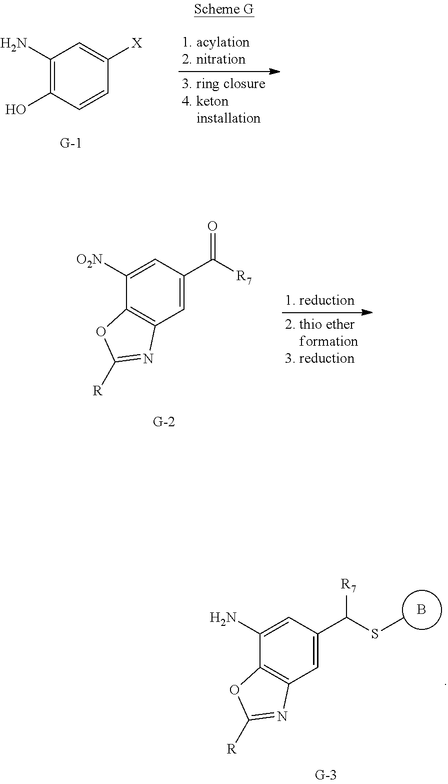

2-Hydroxyanilines halogenated in the 5-position (G-1) can be acylated under standard conditions followed by nitration in the 6-position. Subsequent ring closure under dehydration conditions to form the corresponding benzisoxazole, followed by ketone installation using methods such as transition metal mediated reactions employing vinyl ether reagents, leads to the formation of ketones G-2 following enol ether hydrolysis. Ketone reduction to the secondary alcohol using standard conditions followed by thioether formation using thiol-containing heterocycles under conditions such as the Mitsunobu reaction or alcohol activation via sulfonylation leads to the anilines G-3 following reduction of the nitro group.

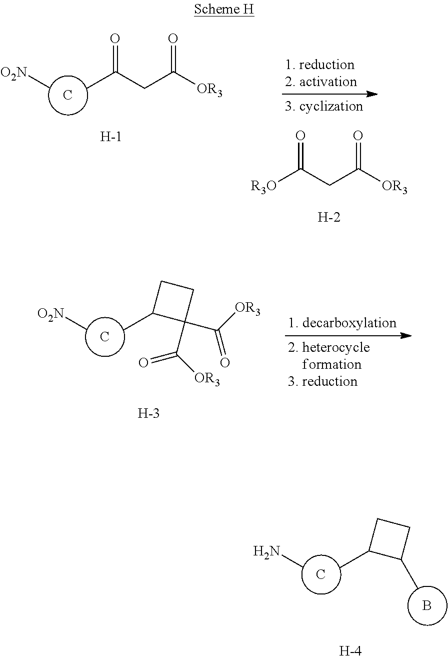

Cyclobutyl containing aniline H-4 can be accessed via reduction of □-keto esters H-1 to the corresponding 1,3-diols using standard conditions followed by bis-activation of the primary and secondary alcohols to, for example, the corresponding primary and secondary halides. Reaction with malonates H-2 leads to the cyclobutanes H-3 via cyclization. Mono-hydrolysis and concomitant decarboxylation leads to esters that can be readily elaborated to heterocycles H-4 as depicted in schemes U-AC following the reduction of the nitro-group under standard conditions.

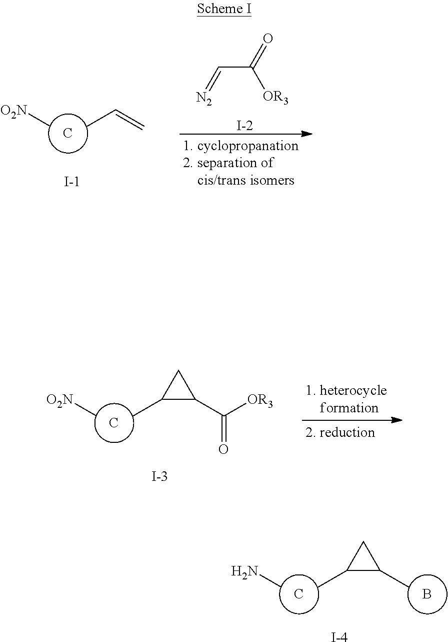

The synthesis of cyclopropane-substituted aniline I-4 can be carried out by cyclopropanation of 3-vinyl nitro benzene I-1 using diazoacetates I-2 under standard conditions to produce the corresponding cyclopropanes as racemic mixtures of geometrical isomers. Following separation of these geometrical isomers, esters I-3 may be converted to heterocycles as depicted in schemes U-AC followed by reduction of the nitro group under standard conditions.

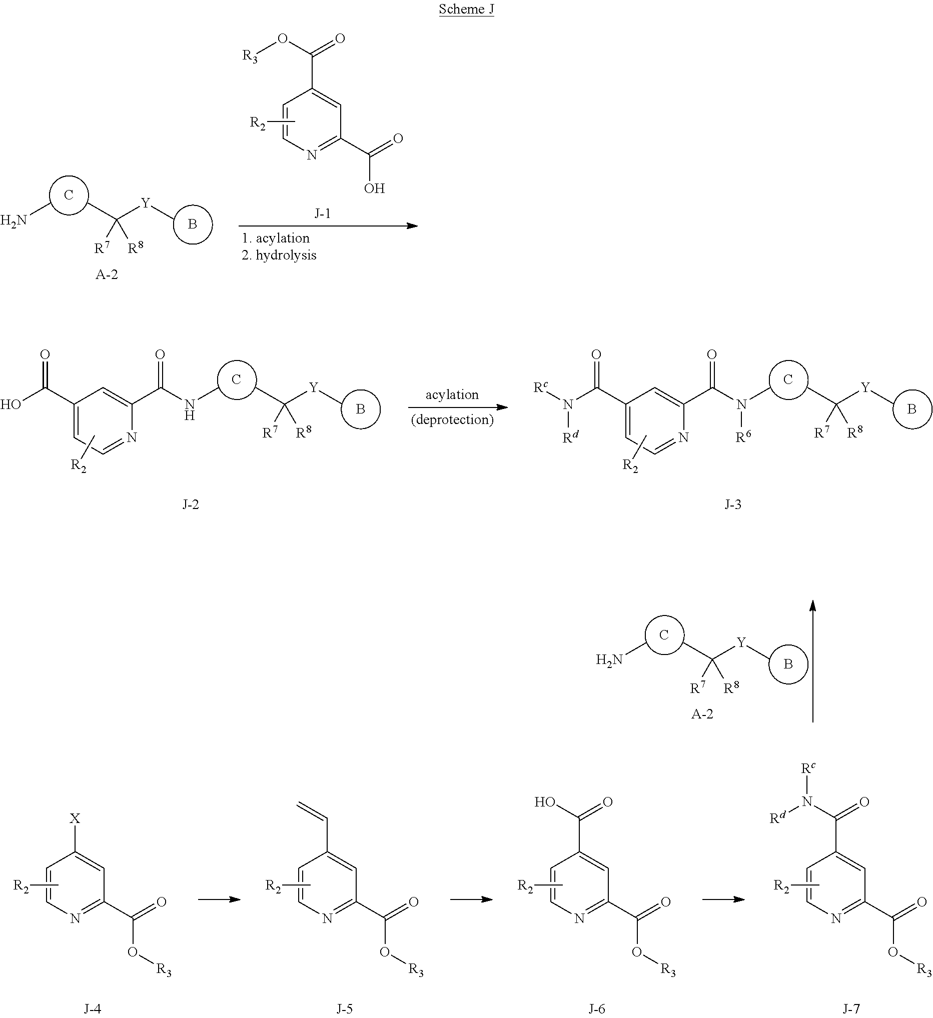

Bis-amides as exemplified by J-3 can be synthesized from intermediates A-2 by two different approaches. In the first approach, amine A-2 can be acylated using pyridine acid esters J-1 followed by hydrolysis of the ester to yield carboxylic acid intermediates J-2. A second acylation step with primary or secondary amines followed by an optional deprotection reaction leads to bis-amide analogs J-3. The second approach elaborates 4-halo pyridines substituted with esters in the 2-position of the pyridine (J-4) to the corresponding vinyl derivatives J-5 using standard methods such as transition metal mediated cross couplings. Subsequently, the vinyl group of J-5 is converted to the corresponding carboxylic acid J-6 under standard conditions which can be elaborated to the corresponding amides J-7. The ester group of J-7 can be used directly as a functional group to be converted to the corresponding amide J-3 using amine A-2 under standard conditions such as the use of trimethyl aluminum.

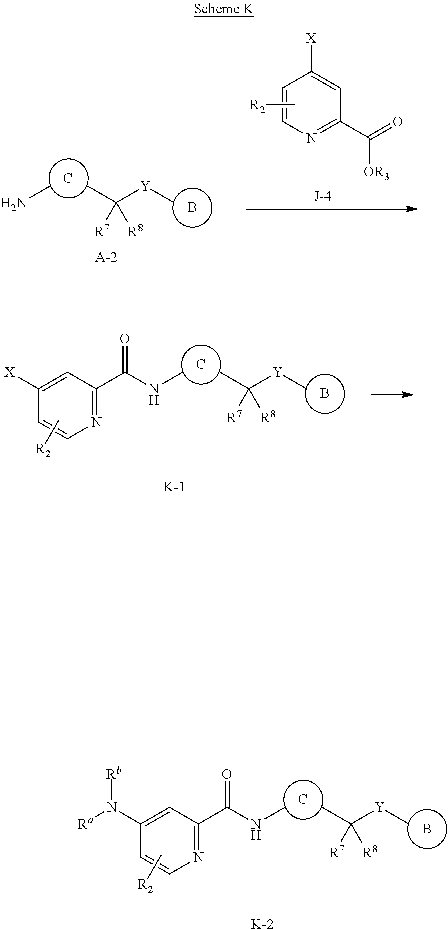

Pyridine-2-esters with a leaving group in the 4-position of the pyridine ring (J-4) may be used to acylate amines A-2 directly using activating reagents such as trimethyl aluminum to provide amides K-1. Conversion of the activated pyridine K-1 to the corresponding amine-substituted product K-2 can be carried out directly with amines at elevated temperature.

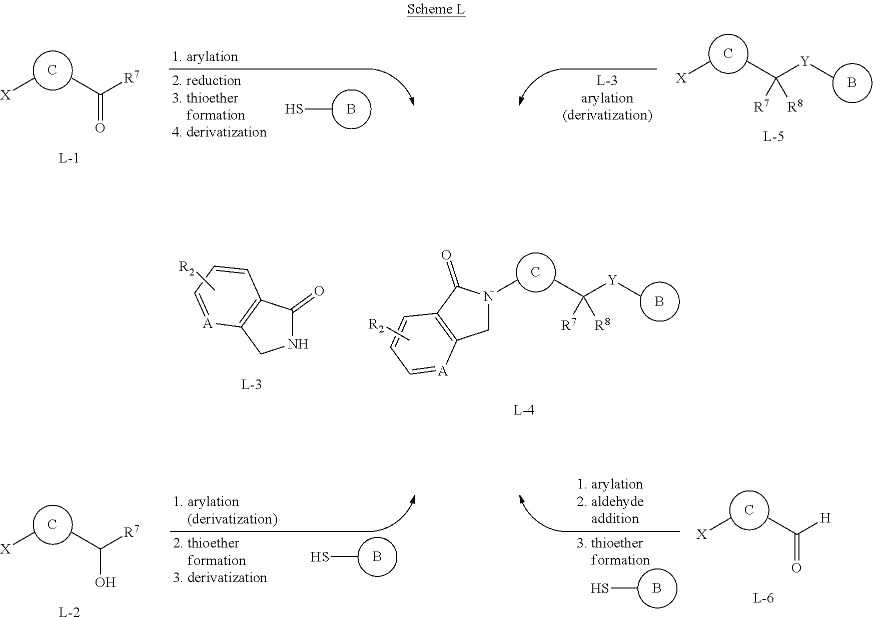

A 3-acyl substituent aryl bromide L-1 or the corresponding secondary alcohol L-2 may be used as a reagent in an arylation reaction to derivatize lactam L-3 under standard conditions. Following reduction of the ketone to the secondary alcohol (in cases where L-1 was used as a starting material), and thioether formation under conditions such as the Mitsunobu reaction, final products L-4 are obtained that optionally can be derivatized further.

Analogs L-4 also can be obtained from appropriately functionalized intermediates, such as halogenated arenes L-5 that can be reacted with lactams L-3 and optional subsequent derivatization. Appropriately functionalized aryl aldehydes L-6 also can be used to arylate lactams L-3 followed by nucleophilic addition to the aldehyde and subsequent reaction of the intermediate secondary alcohol to the targets L-4 under conditions such as the Mitsunobu reaction.

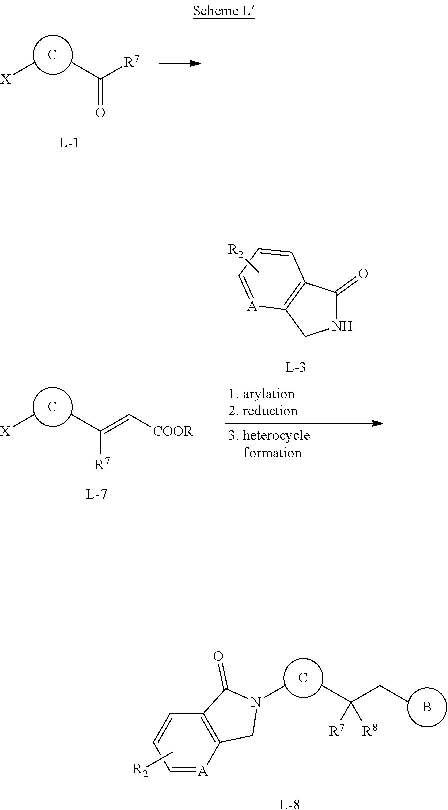

Lactam analogs such as L-8 may be obtained by elaborating ketones L-1 to esters L-7 under conditions such as the Wittig reaction. Appropriately functionalized aryls L-7 can be utilized to arylate lactams L-3 under standard conditions such as transition metal mediated reactions. Reduction of the produced olefin leads to intermediates that can be advanced as depicted in schemes U-AC to heterocycle-containing targets M-2.

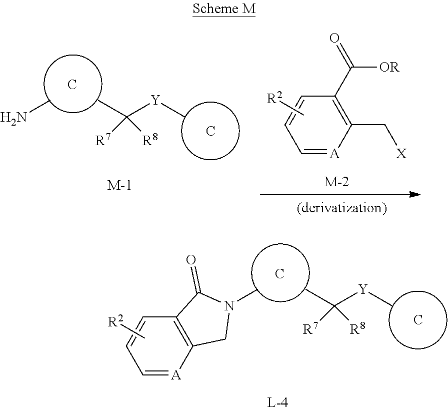

Lactam analogs L-4 also can be prepared by reacting amino arenes M-1 with aryl esters containing a suitably functionalized ortho-methyl substituent such as a halomethyl group. The obtained lactams may optionally be further derivatized to yield targets L-4.

Various methods for connecting rings B and C also are described as follows.

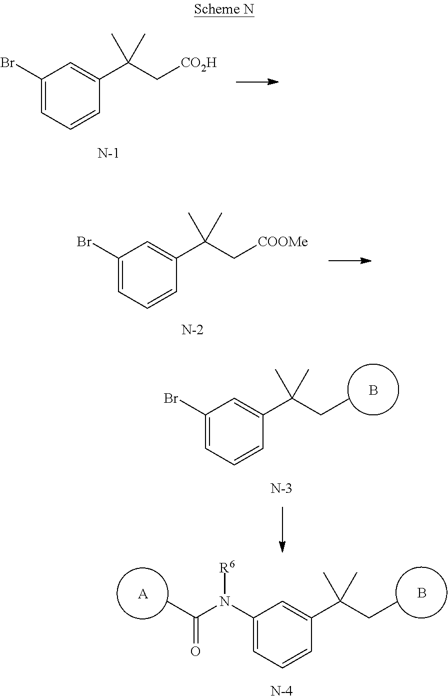

Heterocyclic intermediates featuring a gem-dimethyl moiety can be accessed from the readily available aryl propionic esters or acids applying chemistry as outlined in the schemes U-AC below.

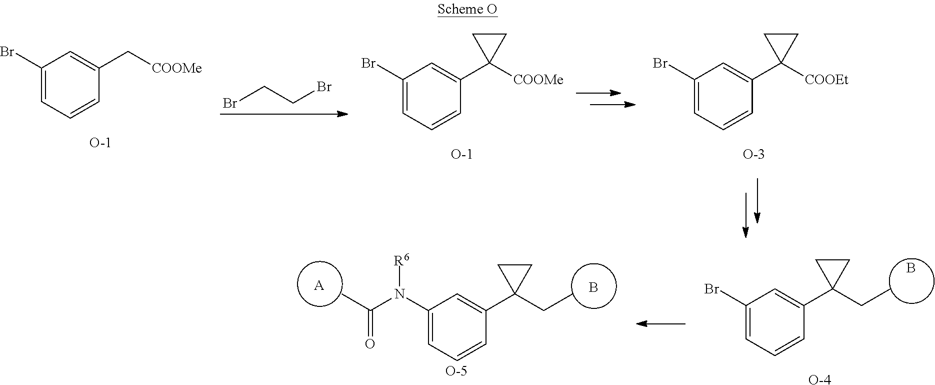

Heterocyclic intermediates featuring a cyclopropane moiety can be accessed from the readily available aryl acetic esters applying standard chemistry to introduce a cyclopropane moiety by alkylation using reagents such as 1,2-dibromoethane. Standard chemistry, such as the Arndt-Eistert homologation followed by conversion of the ester to heterocycles as outlined in schemes U-AC below, can be applied to extend the ester moiety by a CH₂-group.

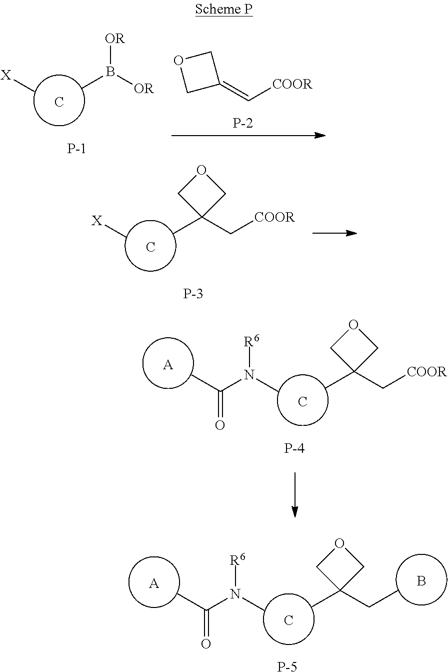

Heterocyclic intermediates featuring an oxetane moiety can be accessed by reaction of readily available aryl boronic acids with appropriate functional groups such as halogens and 2-(oxetan-3-ylidene)acetic esters under conditions of the Hayashi reaction. Subsequent derivatization of the aryl halides for example under transition metal-mediated conditions allows access to the corresponding amides or lactams that can be further elaborated to heterocycles as outlined in schemes U-AC below.

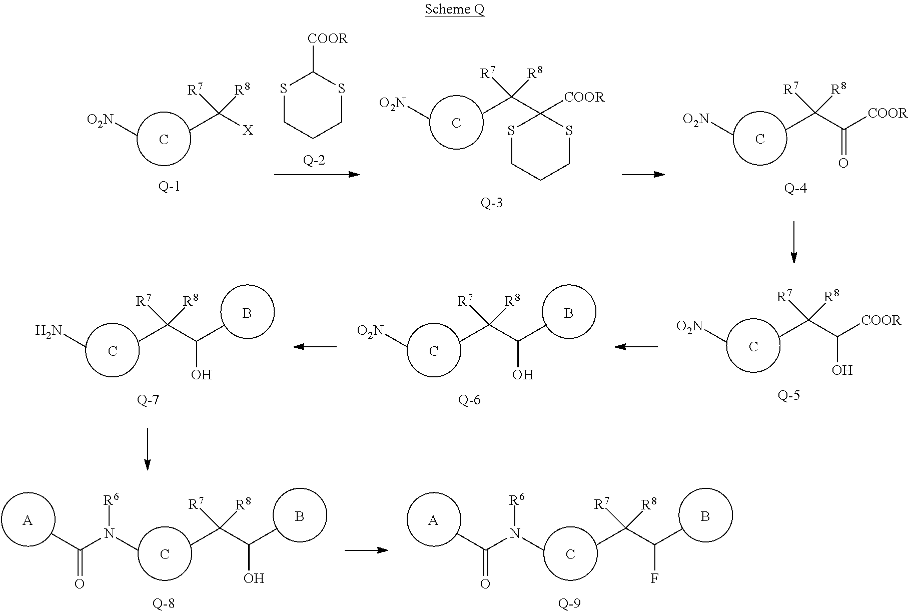

Certain targets featuring fluorination can be accessed, for example from nitro-substituted benzyl halides by reaction with dithiane acetic esters, to obtain the corresponding aryl propionic esters. Hydrolysis of the dithiane under standard conditions leads to the corresponding a-keto-esters that can be reduced to the amino substituted intermediate containing a secondary alcohol. Amine acylation followed by conversion of the alcohol to the corresponding fluoride leads to the target analogs.

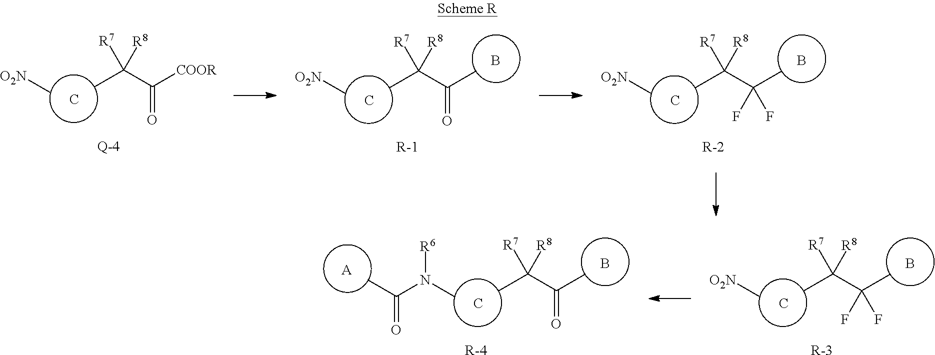

Other heterocyclic targets featuring fluorination can be accessed directly from the alpha-keto-esters that can be converted to heterocycles as outlined in schemes U-AC below. Subsequent conversion of the carbonyl group to the gem-difluoride moiety followed by nitro reduction and acylation provides the desired fluorinated analogs.

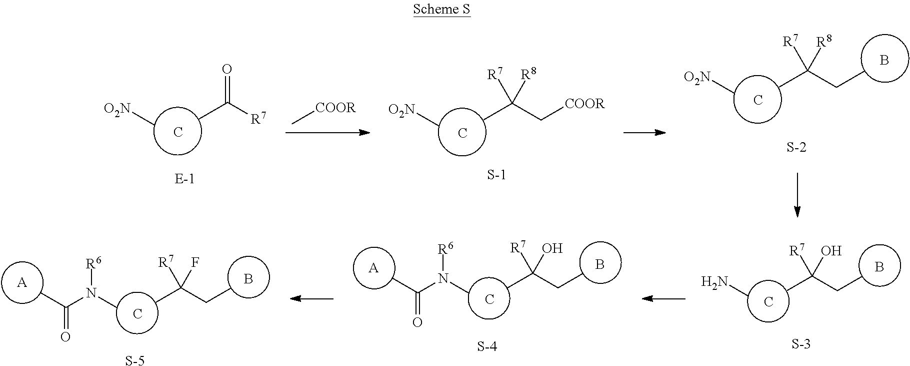

Other heterocyclic targets featuring fluorination can be accessed, for example by addition of acetates to nitro-aryl ketones under Claisen condensation conditions. The esters can be converted to heterocycles as outlined in schemes U-AC below. Nitro reduction followed by acylation and conversion of the tertiary alcohol to the corresponding fluoride yields the desired fluorinated analogs.

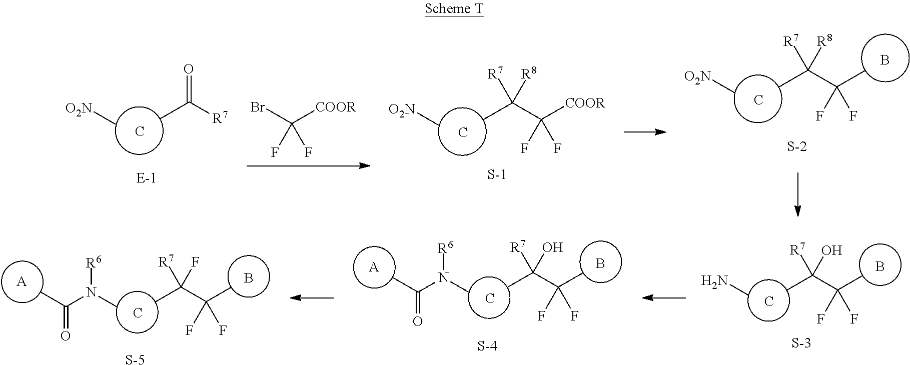

Other heterocyclic targets featuring fluorination can be accessed, for example by reaction of nitro-aryl ketones with bromo-di fluoro acetates under halogen-metal exchange conditions, to yield the corresponding tertiary alcohols. Subsequent conversion of the esters to heterocycles as outlined in schemes U-AC below followed by nitro reduction leads to amino aryl intermediates. Acylation of said amines followed by conversion of the tertiary alcohol to the corresponding fluoride leads to the trifluoro analogs.

Formation of the heterocycles in the compounds, such as in Ring B, can be carried out as described below.

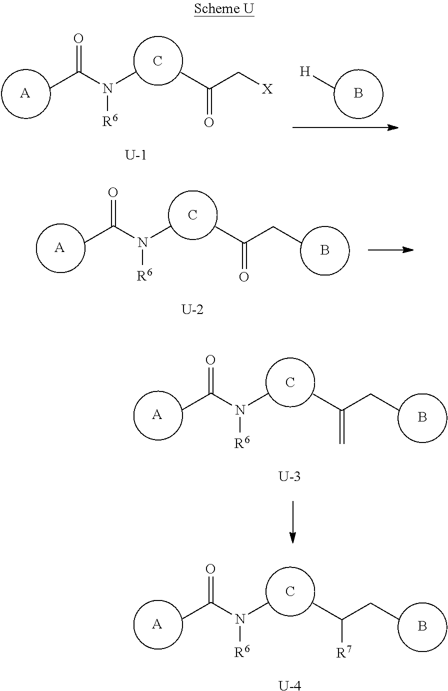

Heterocyclic analogs linked via one of their heteroatoms can be obtained by reaction of the heterocycles with halomethyl ketones 1-2, followed by subsequent conversion of the carbonyl group to the corresponding olefins under conditions such as the Wittig reaction and hydrogenation of the tis obtained olefins to the final targets.

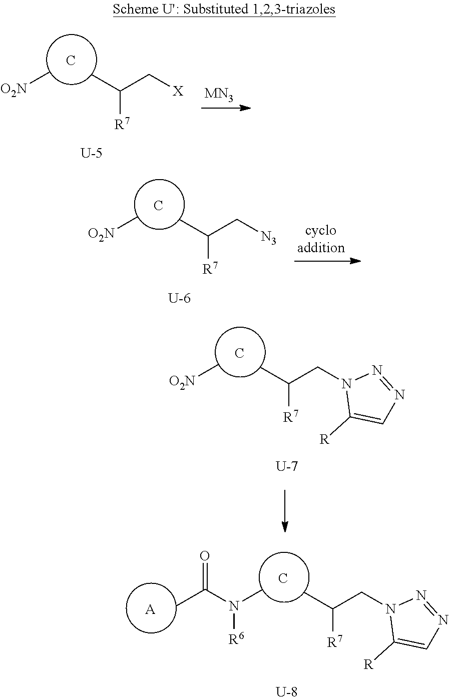

Analogs featuring a 5-substituted 1,2,3-triazole moiety U-8 can be synthesized by reacting sodium azide with nitroarenes U-5 substituted with an ethyl group functionalized with a leaving group such as a halide or sulfonate. The thus-obtained azido intermediates U-6 can be elaborated to substituted triazoles U-7 that can be advanced to the final analogs U-8 following nitro reduction under standard conditions (such as Palladium on carbon in a hydrogen atmosphere) and acylation using for example acids A-1.

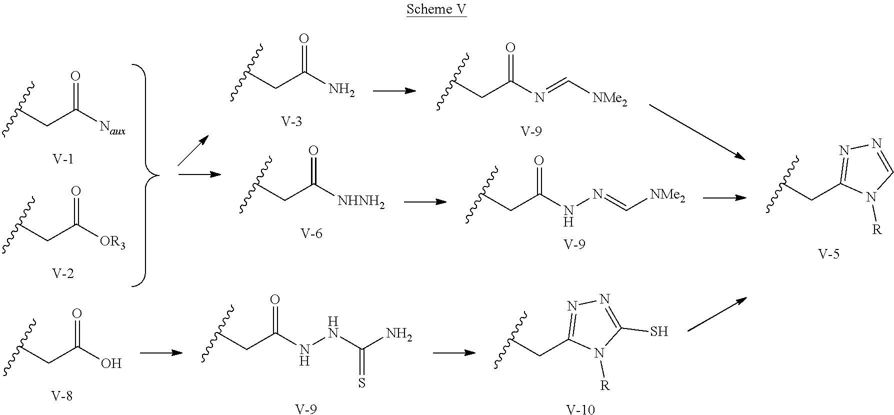

1,2,4-Triazole-containing analogs V-5 can be accessed from the corresponding acyl precursors through alternate routes. Amides V-1 or esters V-2 can be converted directly to the corresponding amides V-3 that can be converted to the N-((dimethylamino)methylene)amides V-4. Reaction of V-4 with hydrazine allows access to 1,2,4-triazole analogs V-5. Amides V-1 or esters V-2 also can be converted to hydrazides V-6 that may be elaborated to an N,N-dimethylformohydrazonamide V-7 and subsequently cyclized using primary amines. Alternatively, hydrazides V-6 may be converted to the corresponding 4-methyl-1,2,4-triazole-3-thiol V-10 by use of methyl isothiocyanate that can be desulfurized to the 4-methyl-1,2,4-triazole V-5 under a variety of conditions such as using NaNO₂/HNO₃ or hydrogen peroxide. In a third method, carboxylic acid V-8 can be condensed to thiosemicarbazide V-9, which will cyclize under basic conditions to form 4-methyl-1,2,4-triazole-3-thiol V-10.

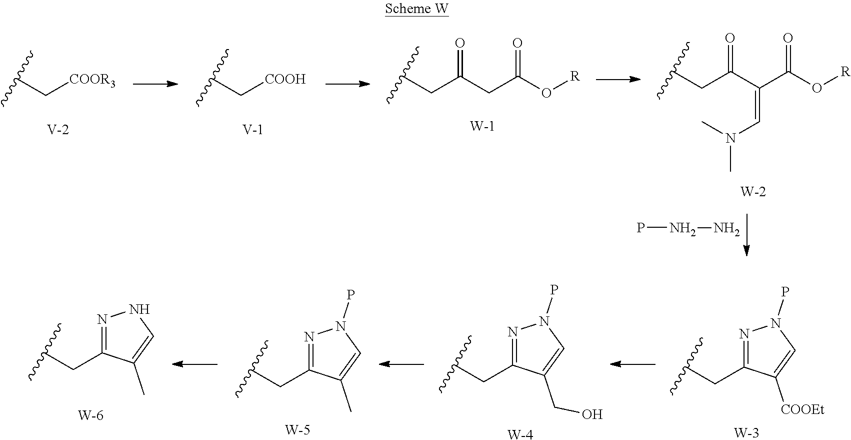

3,4-Disubstituted pyrazoles W-6 can be synthesized from esters V-2 or acids V-1 by elaboration to the corresponding b-keto esters W-1 using approaches such as acylation of mono-alkyl malonates followed by decarboxylation. Conversion to the 2-((dimethylamino)methylene)-3-oxobutanoate W-2 allows for the cyclization to the ester substituted pyrazoles W-3 using mono-protected hydrazine derivatives. Final compounds can be obtained by reduction of the ester to the corresponding hydroxymethyl (W-4) or methyl pyrazoles (W-5) and subsequent deprotection.

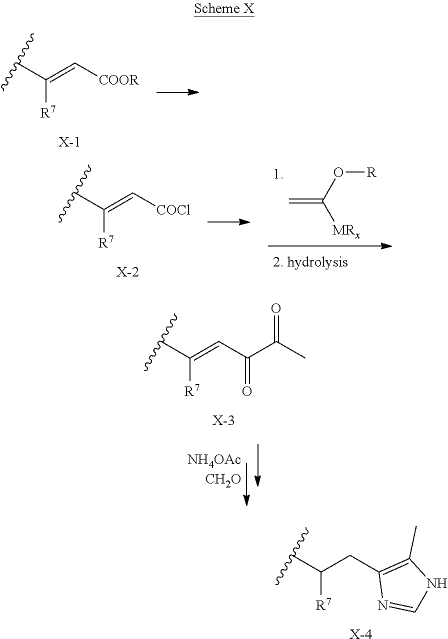

4,5-Disubstituted imidazole analogs X-4 can be accessed through conversions of a,b-unsaturated esters X-1 to acid chlorides X-2 that can be elaborated to the 1,2-diketones X-3 using vinyl ethers under transition metal-mediated conditions followed by hydrolysis. The 1,2-diketones can be advanced to the final compounds X-4 by condensation with reagents such as ammonium acetate and formaldehyde.

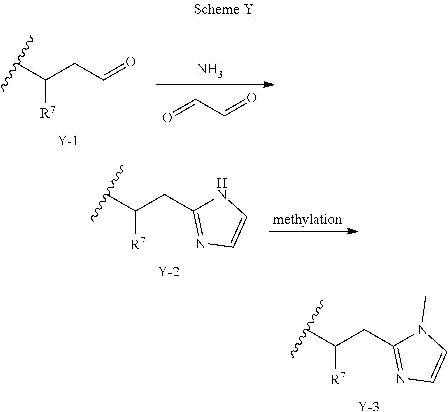

2-substituted imidazoles Y-2 can be obtained from protected aldehydes Y-1 that can be elaborated under condensation conditions with reagents such as ammonia and glyoxal. Subsequent exposure of imidazoles Y-2 to alkylating reagents such as iodomethane or dimethyl sulfate will provide methylimidazole analogs Y-3.

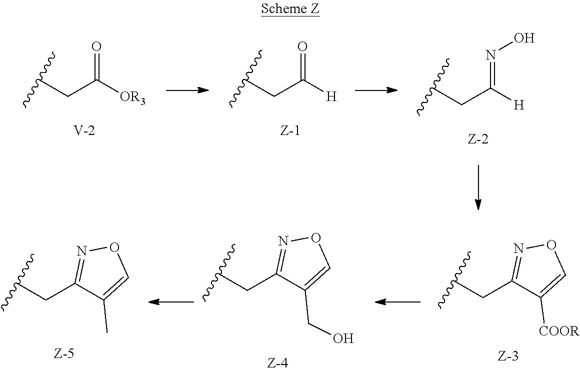

3,4-Disubstituted isoxazoles Z-5 can be synthesized from esters V-2 that can be reduced to the corresponding aldehydes Z-1. Elaboration of the aldehydes to the oxime derivatives Z-2 followed by oxidation with reagents such as N-chlorosuccinimide (NCS) enables a [3+2] cycloaddition with reagents such as dimethylamino acrylates to provide isoxazoles Z-3 that can be reduced to the corresponding methyl analogs Z-5 via the hydroxymethyl derivatives

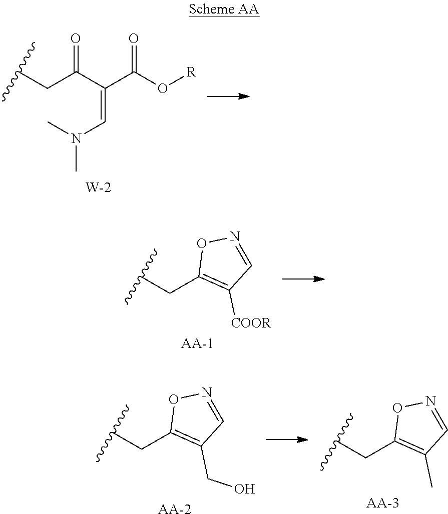

Regio-isomeric 4,5-disubstituted isoxazoles AA-3 may be synthesized from 2-((dimethylamino)methylene)-3-oxobutanoate W-2 and cyclization to the isoxazole AA-1 using reagents such as hydroxylamine. Reduction of the ester moiety to the corresponding methyl derivative AA-3 can be carried out via the hydroxymethyl analog AA-2.

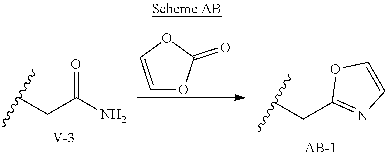

Primary amides represented by V-3 can be allowed to condense with reagents such as 1,3-dioxol-2-one to yield oxazoles AB-1.

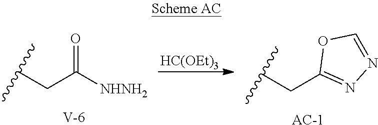

Oxadiazoles AC-1 as disclosed herein can be synthesized by allowing hydrazides V-6 to react with reagents such as triethyl orthoformate.

For compounds disclosed herein, the Ring A moiety can be selected from the group consisting of:

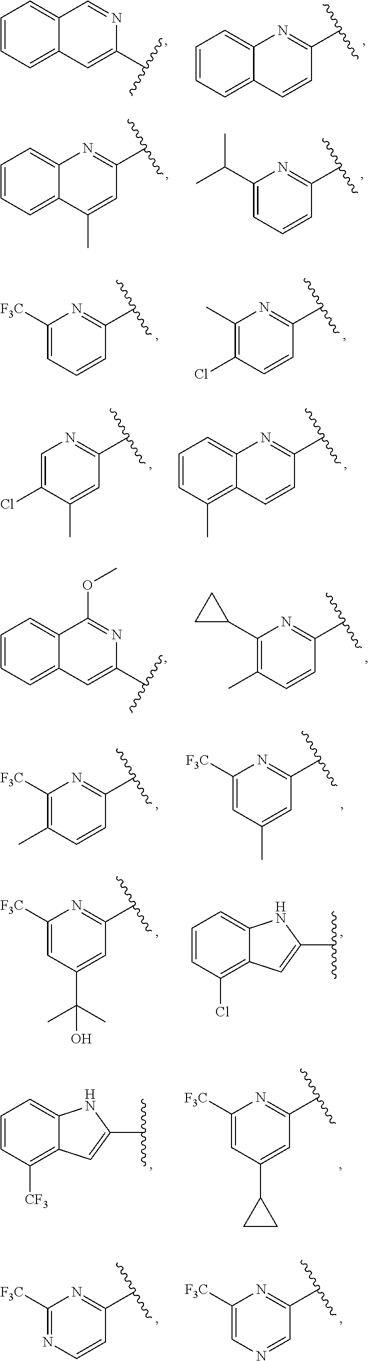

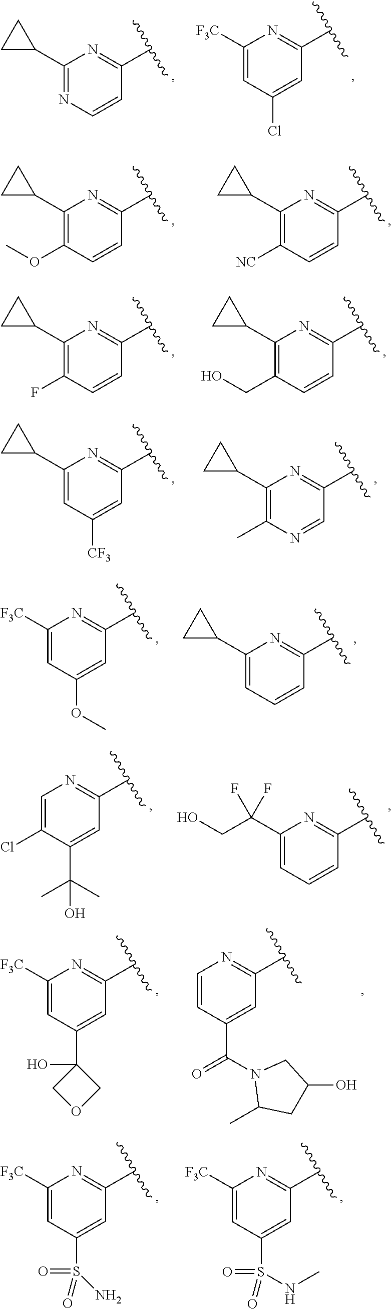

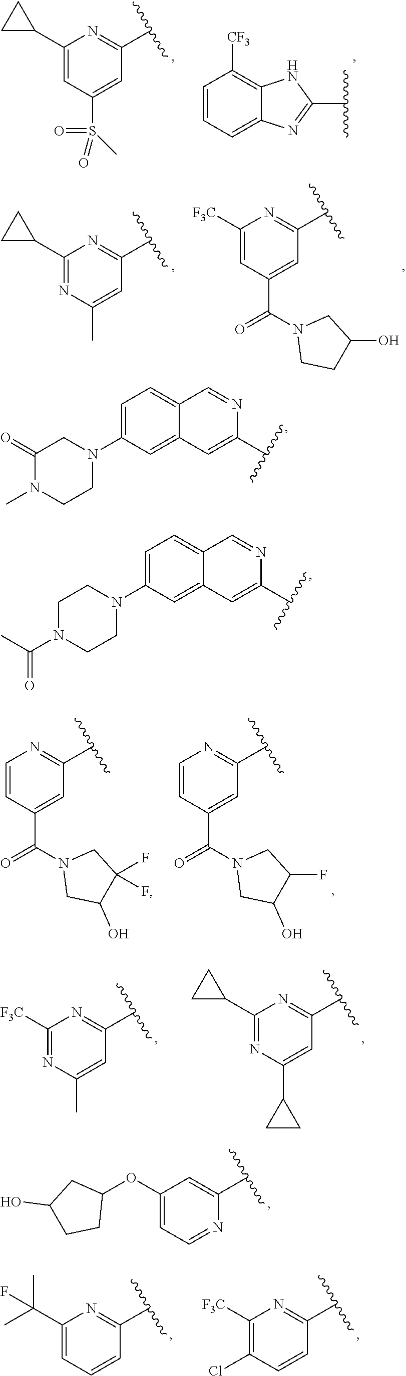

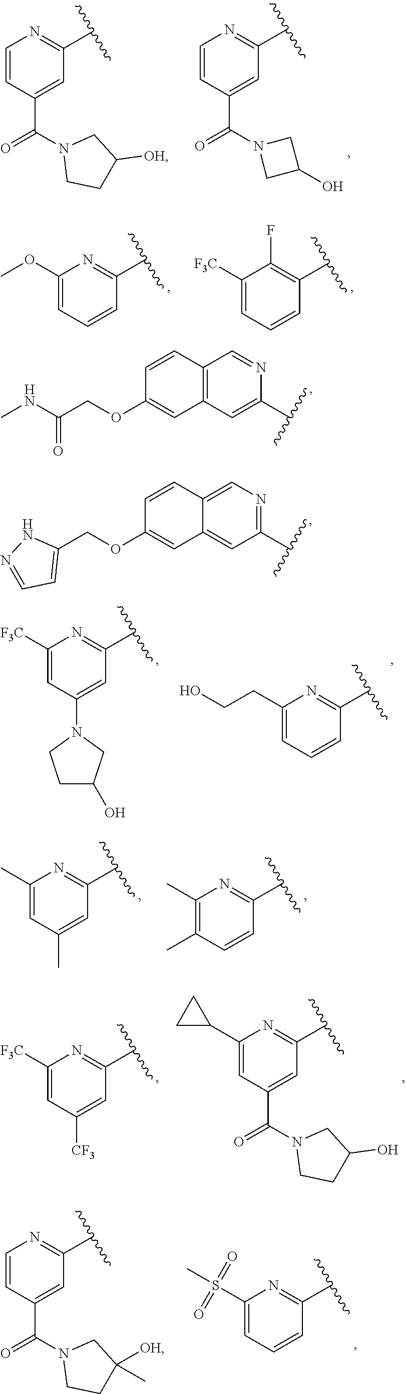

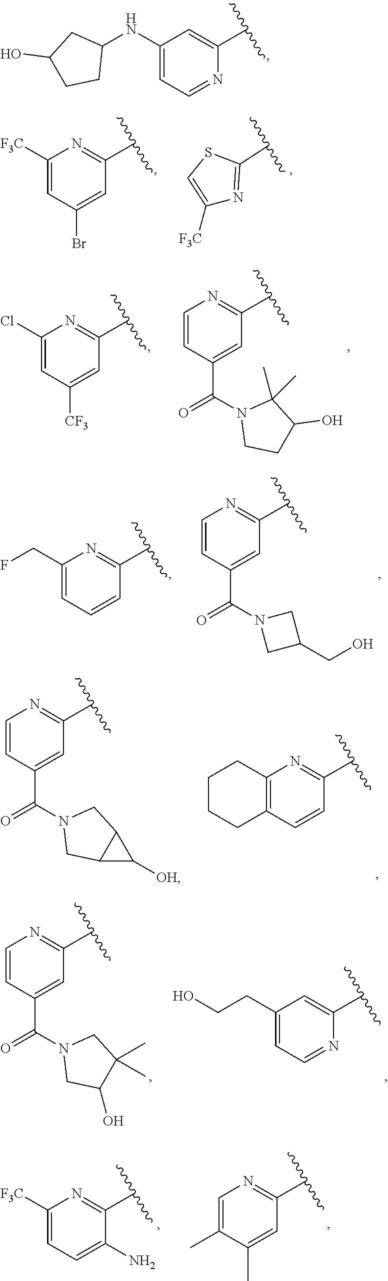

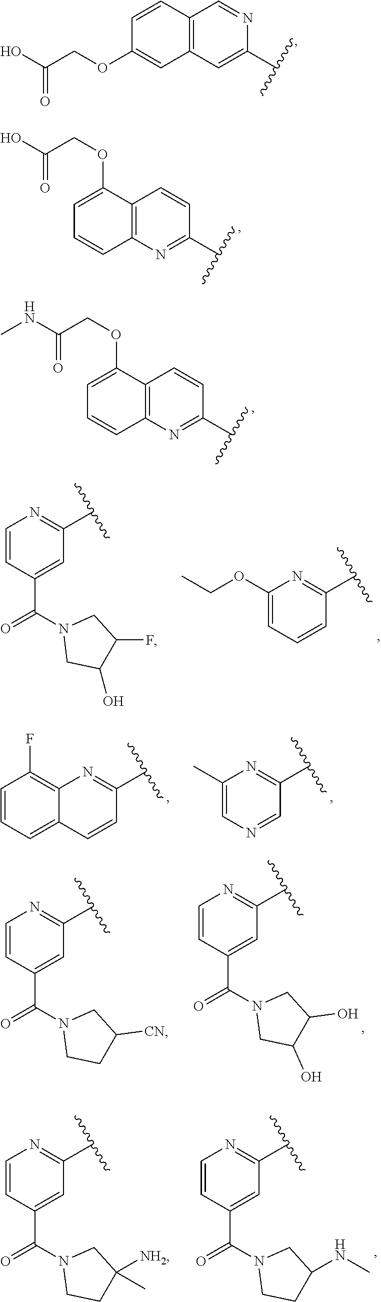

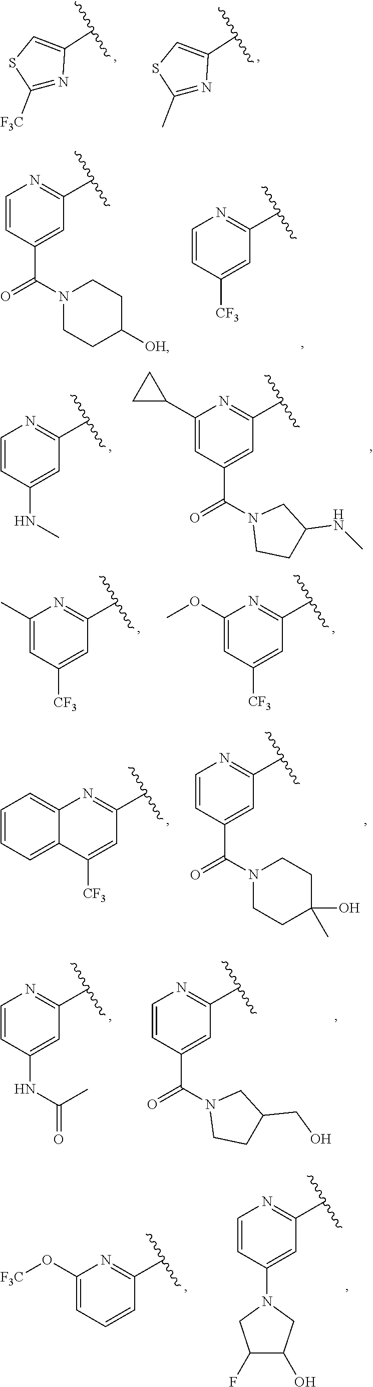

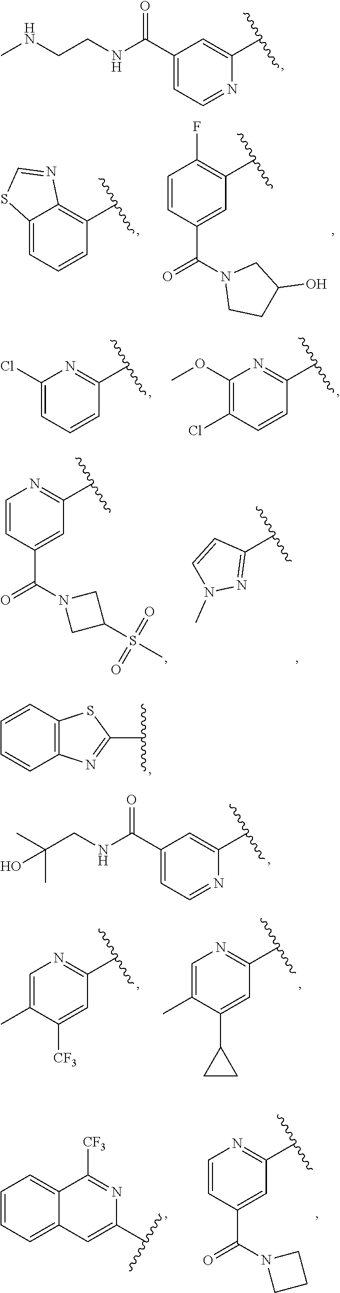

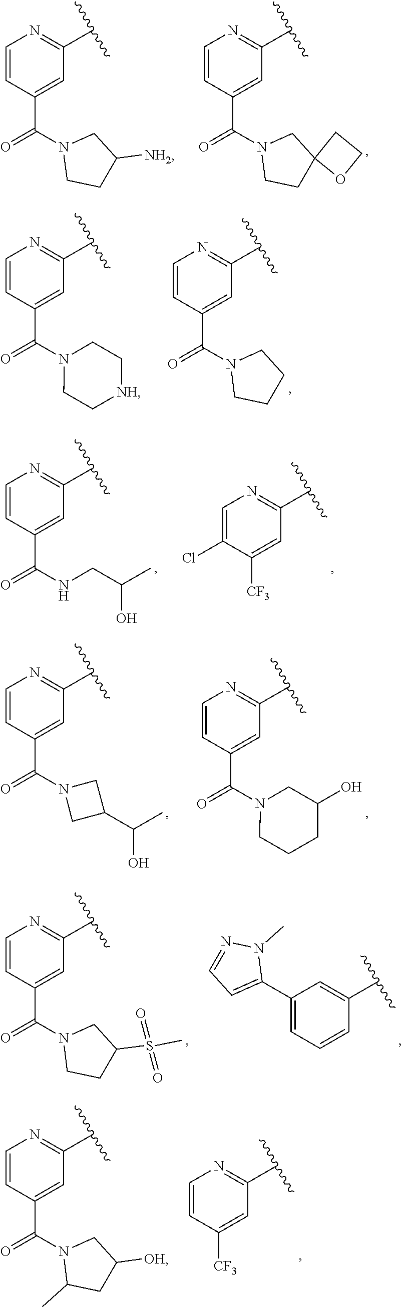

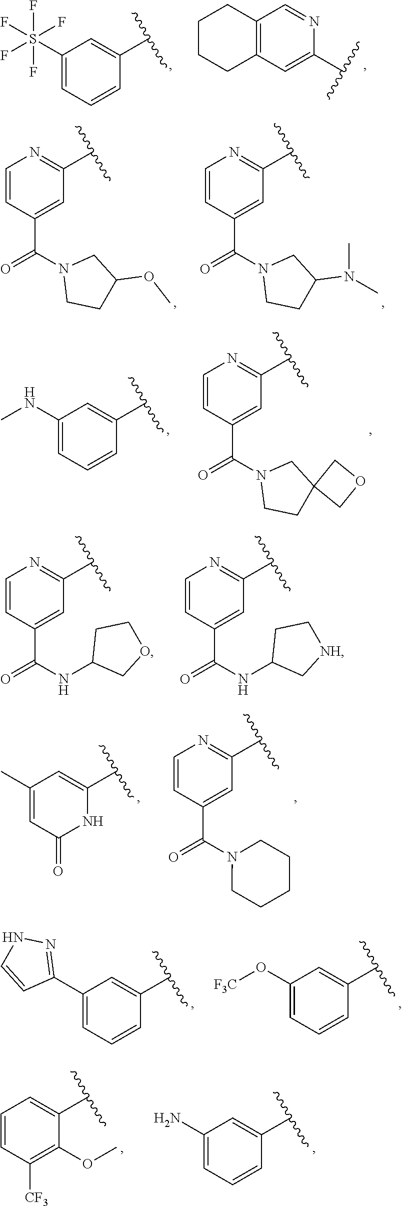

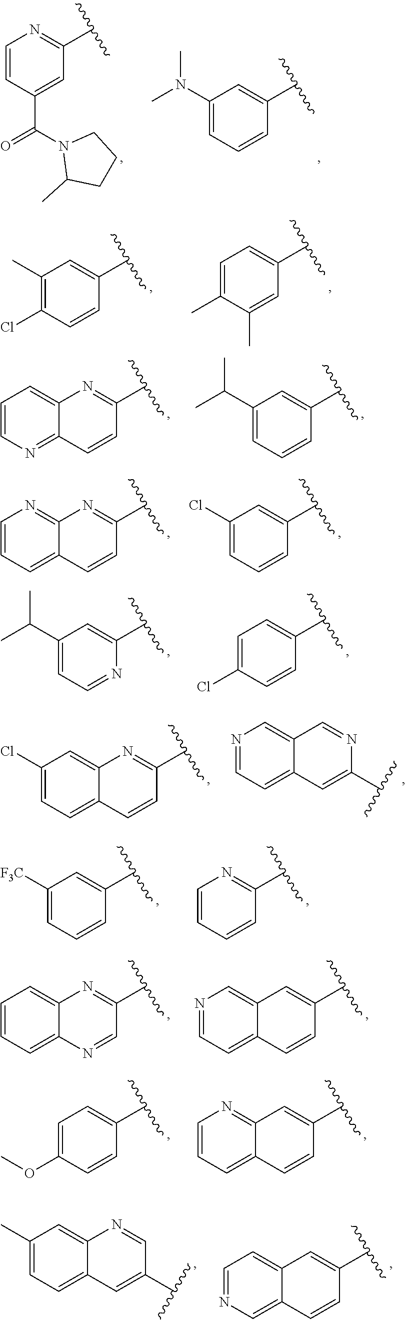

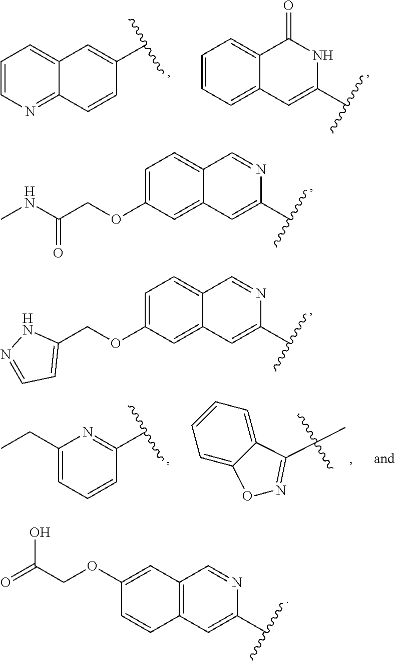

In some embodiments, the Ring A moiety can be selected from the group consisting of:

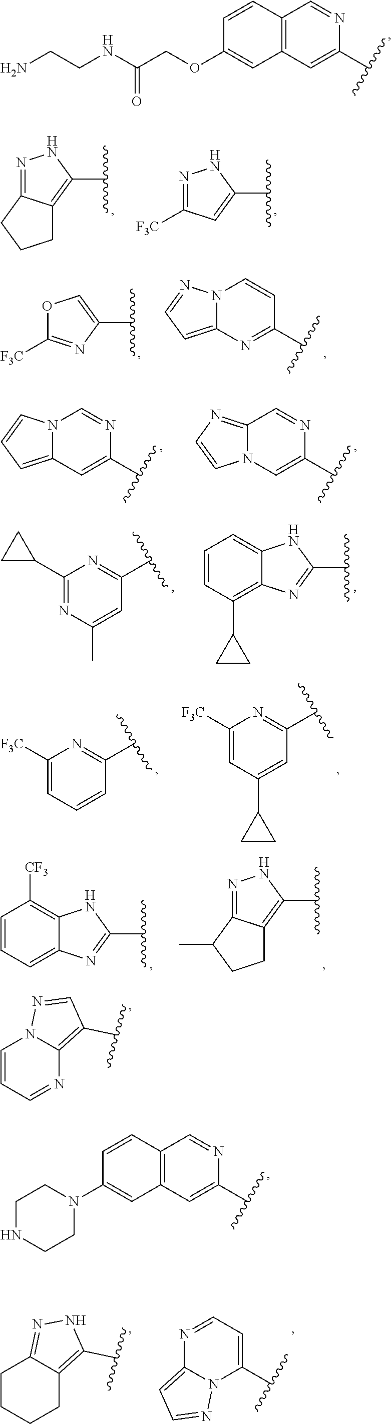

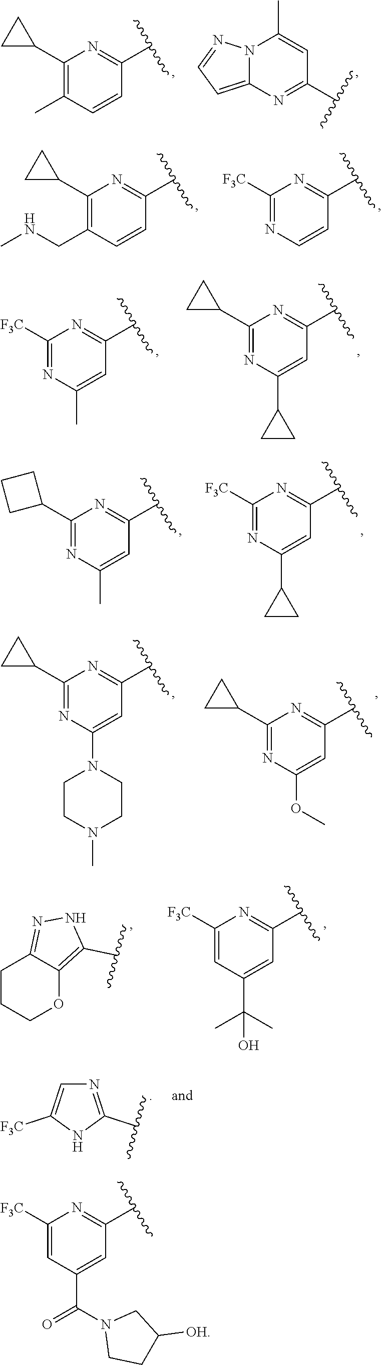

In other embodiments, the Ring A moiety can be selected from the group consisting

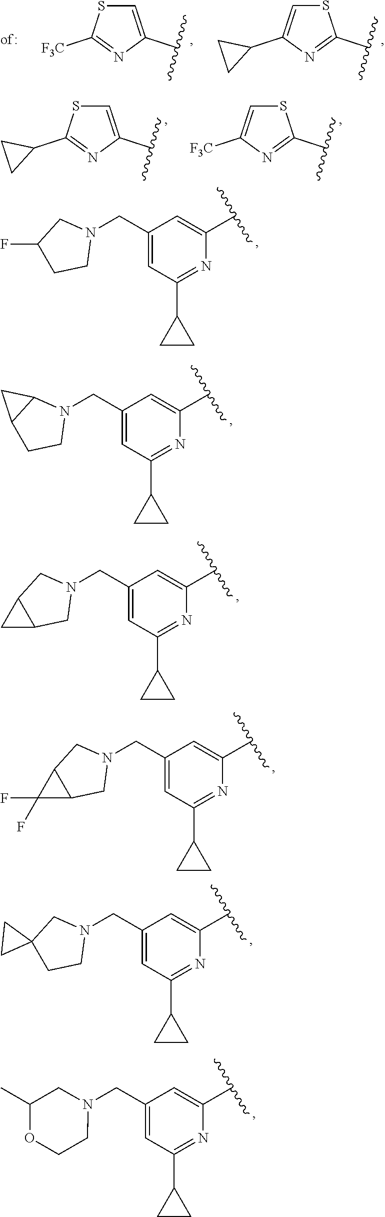

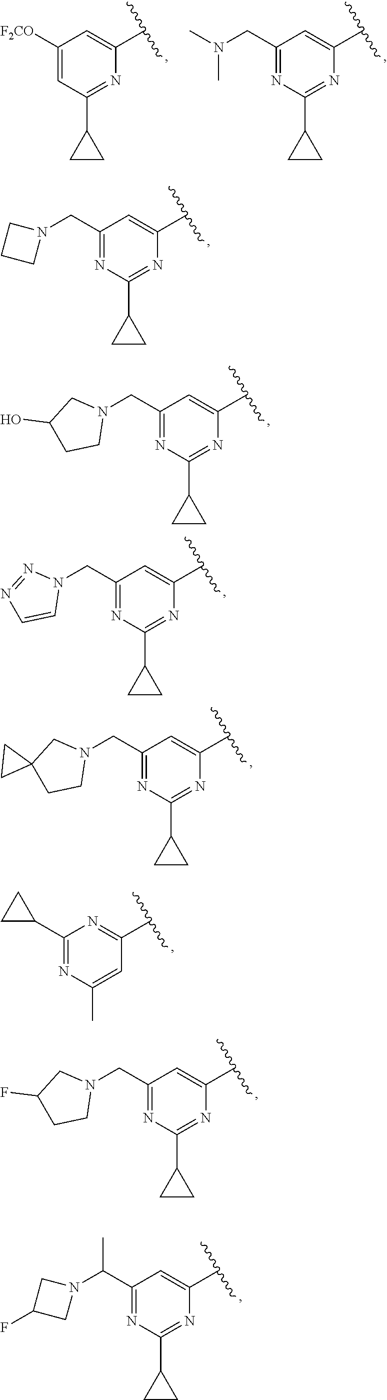

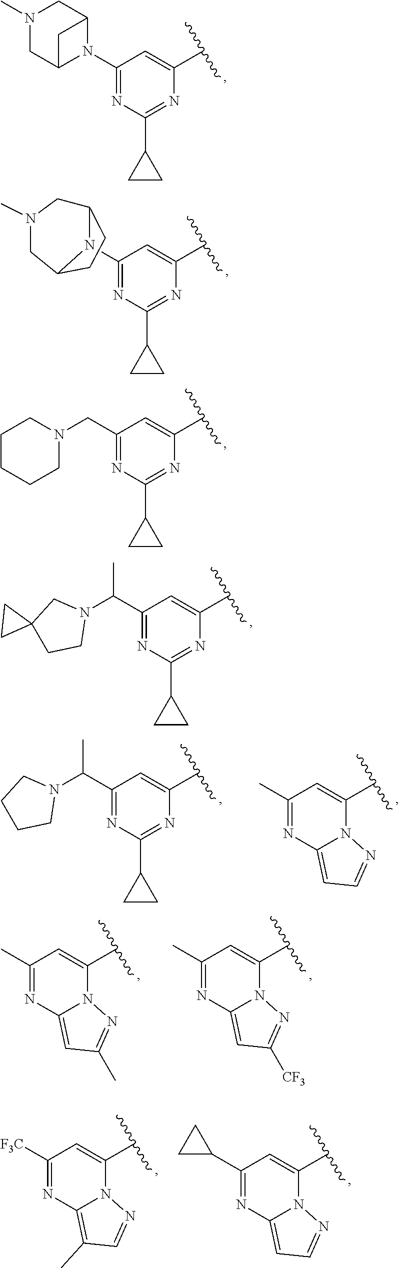

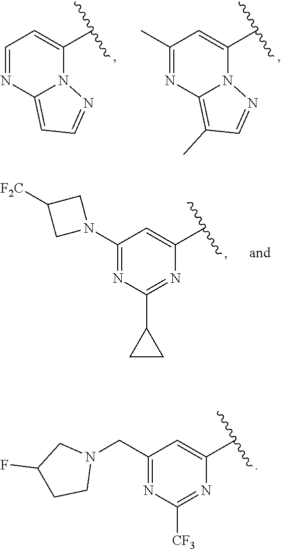

Particular A rings include phenyl, pyridyl, and quinazoline rings, each of which can be optionally substituted. Another particular A ring includes pyrimidyl.

Examples of Ring A substituents (which can be used as R¹¹, R¹², R¹³, and R¹⁴ groups in Formula (I-A) and Formula (I), R^{x} groups in Formula (II-A) and Formula (II), and R^{A} groups in Formula (III-A), Formula (III), Formula (IV), and Formula (IV-ox)) include, but are not limited to, the group consisting of methyl, ethyl, F, Cl, Br, I, —CF₃, —OCF₃, —CH₂F, —NH₂, —NHCH₃, —N(CH₃)₂, 2-propyl, 1-propyl, methoxy, ethoxy, cyclopropyl, 2-hydroxy-prop-2-yl, cyano, —CH₂CH₂OH, —CF₂CH₂OH, —CF₂CH₃, —S(═O)₂—CH₃, —S(═O)₂—NH₂, —S(═O)₂—NHCH₃, —OC H₂-(C═O)—NH₂, —OCH₂-(C═O)—NHCH₃, —OCH₂COOH, —OCH₂C(═O)NH₂, —OCH₂C(═O)NHCH₃, —CONHCH₃, —C(═O)NHCH₂CH₂NH₂, —C(═O)NHCH₂CH₂NHCH₃, —C(═O)NHCH₂C(CH₃)₂OH, —C(═O)NHCH₂CH(CH₃)OH, —SF₅,

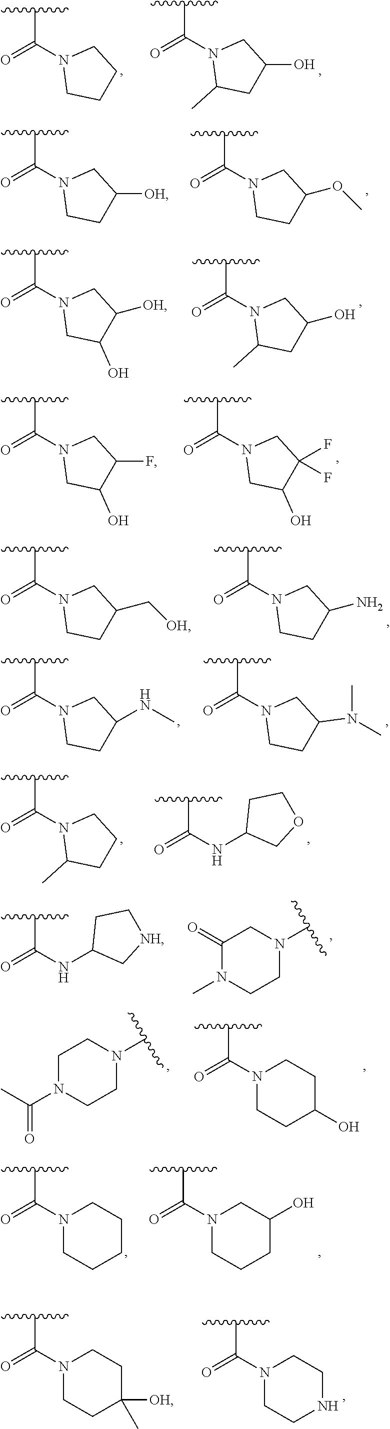

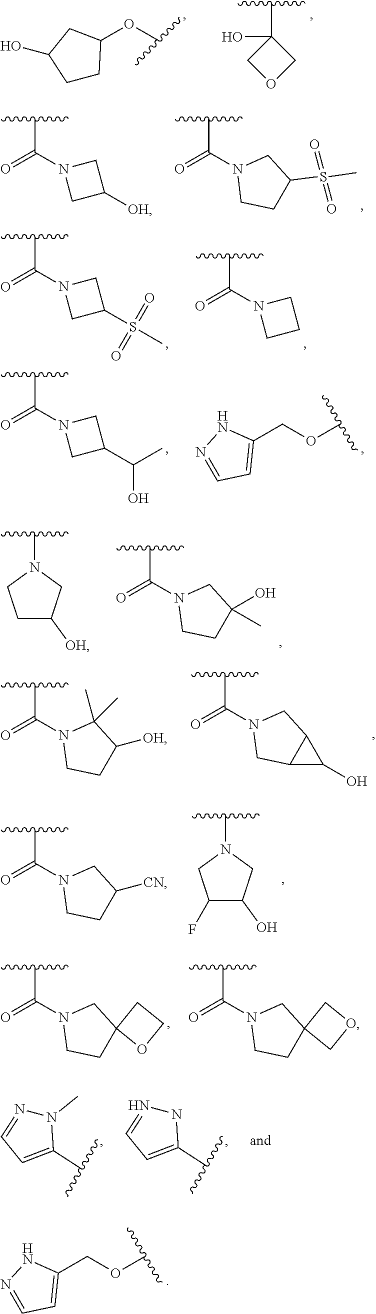

Further examples of Ring A substituents (which can be used as R¹¹, R¹², R¹³, and R¹⁴ groups in Formula (I-A) and Formula (I), R^{x} groups in Formula (II-A) and Formula (II), R^{A} groups in Formula (III-A) and Formula (III), and R^{A} groups in Formula (IV) and Formula (IV-ox)) include F, CF₃, and cyclopropyl. Additional examples of Ring A substituents (which can be used as R¹¹, R¹², R¹³, and R¹⁴ groups in Formula (I-A) and Formula (I), R^{x} groups in Formula (II-A) and Formula (II), R^{A} groups in Formula (III-A) and Formula (III), and R^{A} groups in Formula (IV) and Formula (IV-ox)) include

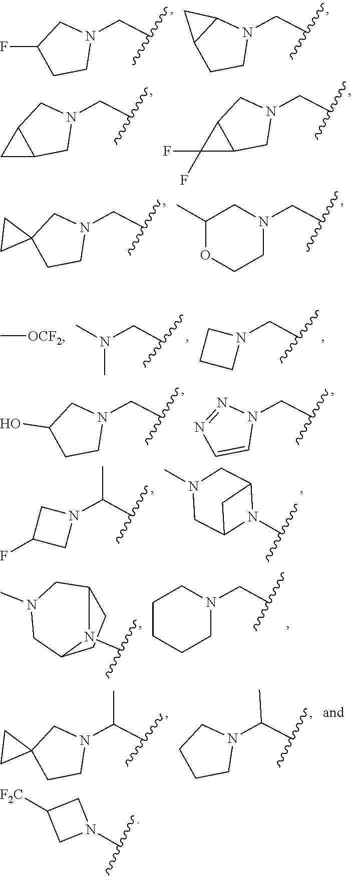

For compounds disclosed herein having a lactam ring, such as those prepared in Scheme L, Scheme L′, or Scheme M above, and for pharmaceutical compositions disclosed herein and for methods disclosed herein using compounds having a lactam ring, the portion of the compound comprising the lactam ring can be selected from the structures described below. These structures comprising a lactam ring arise when the moiety

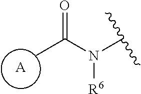

is

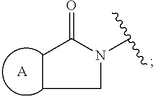

when the moiety

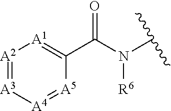

is

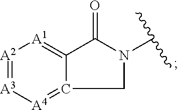

when the moiety

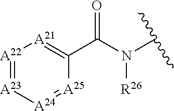

is

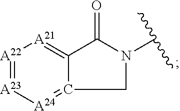

or when the compound contains the moiety

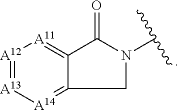

In another embodiment, when the compound has a lactam ring, the portion of the compound comprising the lactam ring can be selected from the group consisting of:

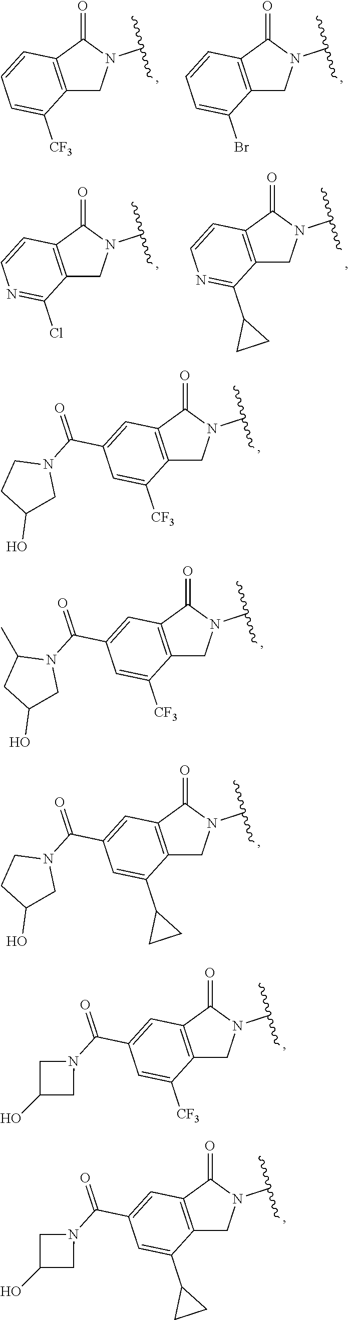

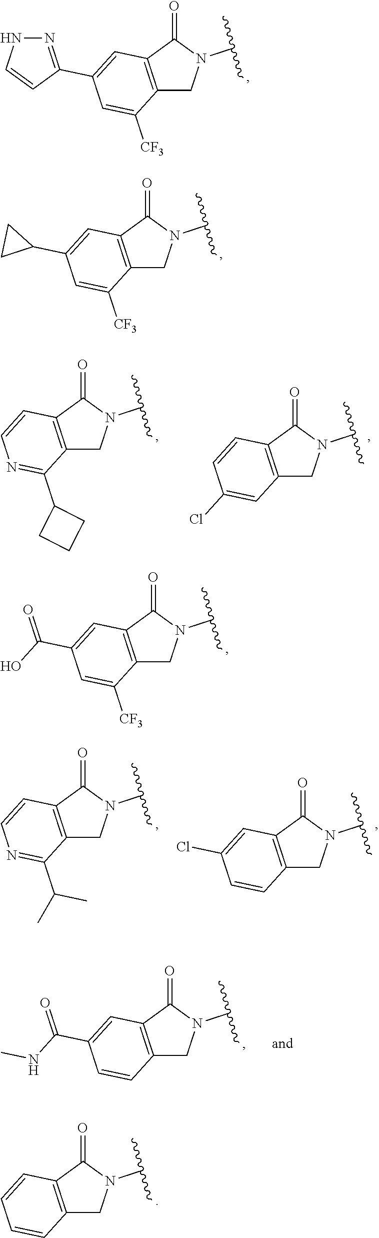

In another embodiment, when the compound has a lactam ring, the portion of the compound comprising the lactam ring can be selected from the group consisting of:

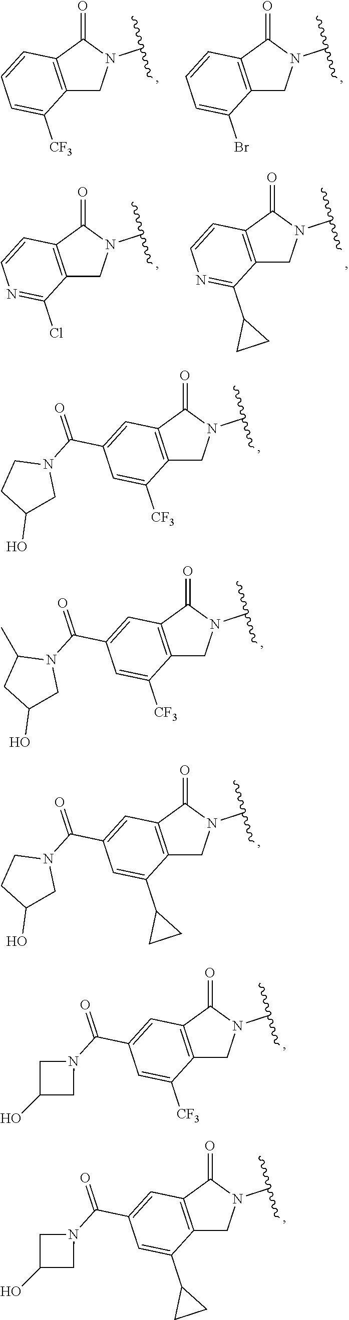

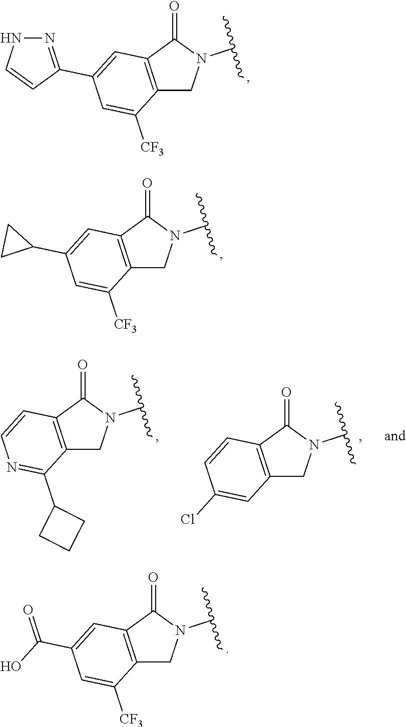

In another embodiment, when the compound has a lactam ring, the portion of the compound comprising the lactam ring can be selected from the group consisting of:

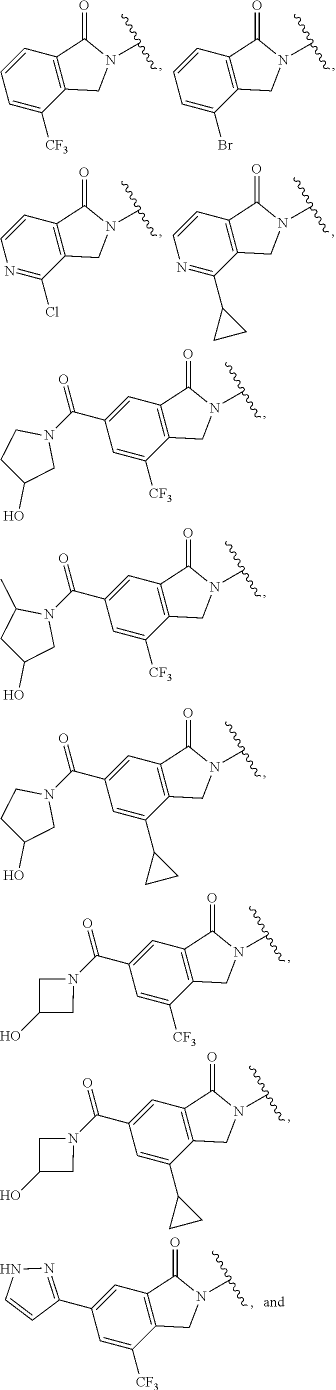

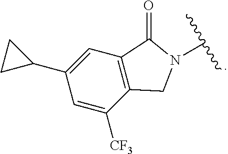 [SMILES: *N1Cc2c(cc(C3CC3)cc2C(F)(F)F)C1=O]

In another embodiment, when the compound has a lactam ring, the portion of the compound comprising the lactam ring can be selected from the group consisting of:

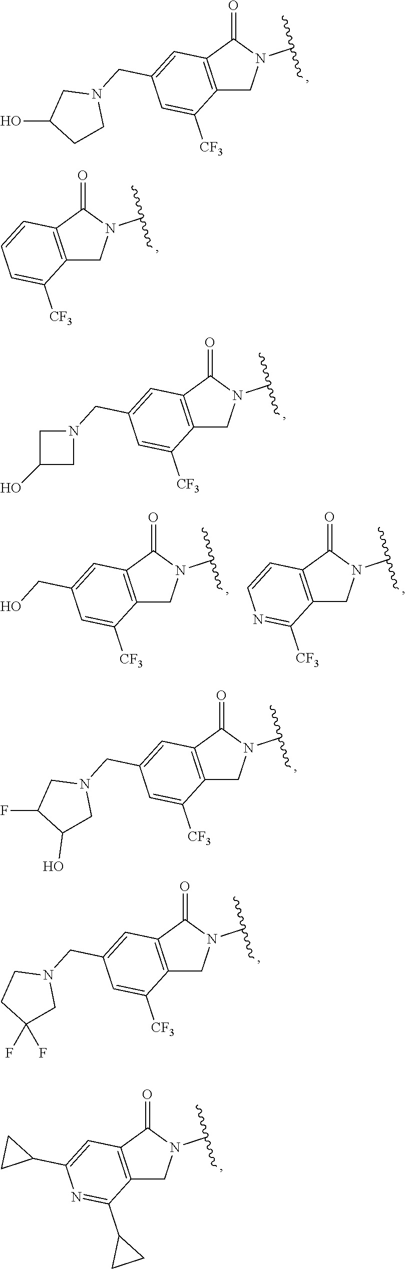

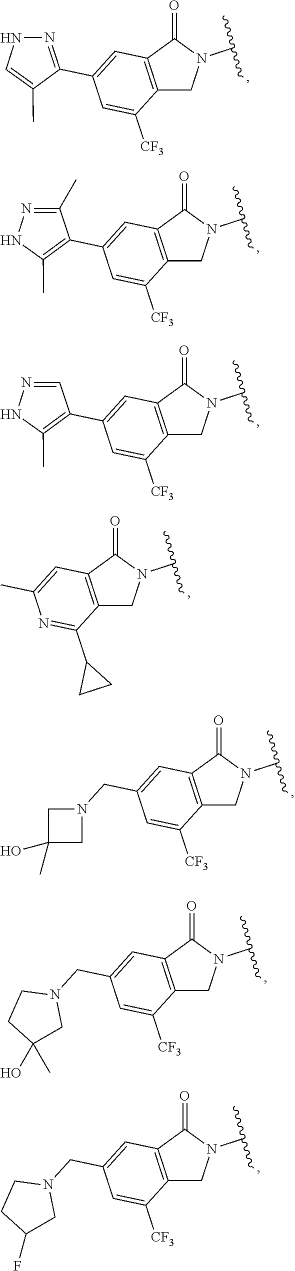

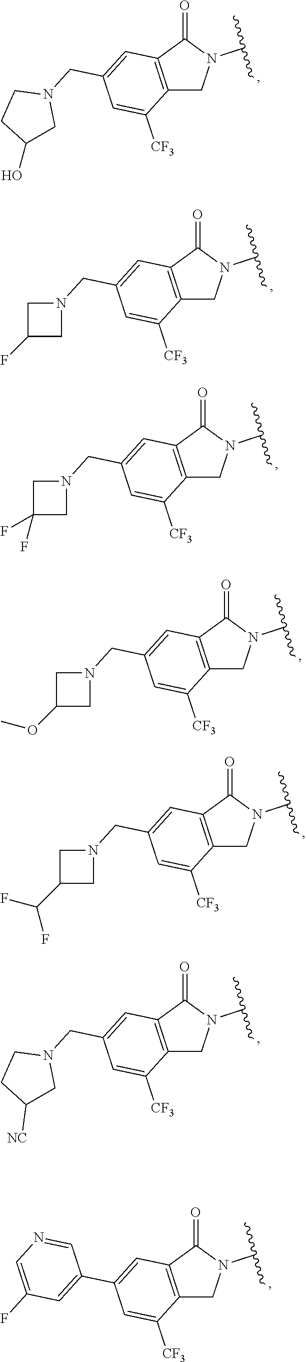

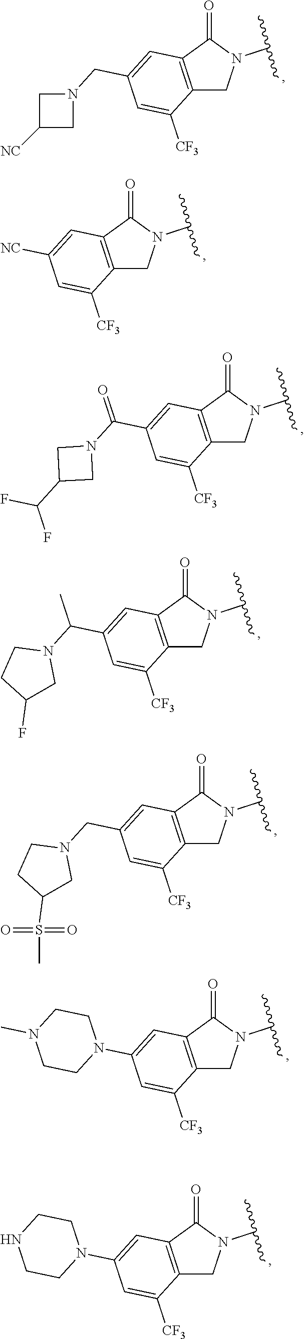

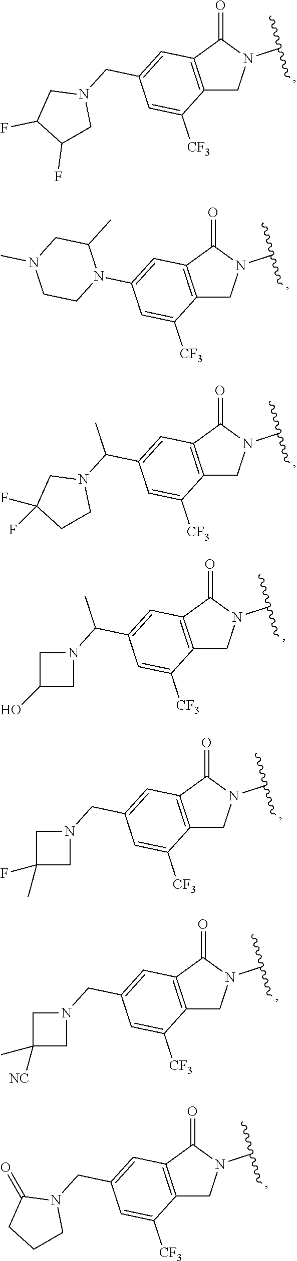

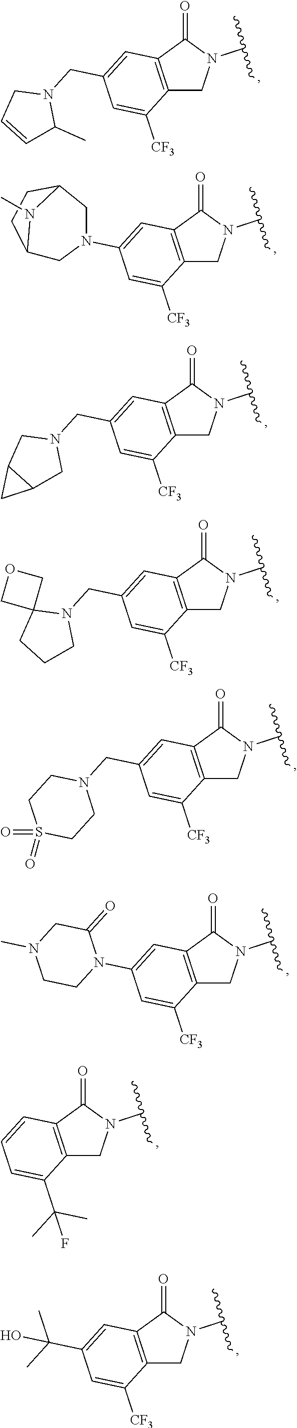

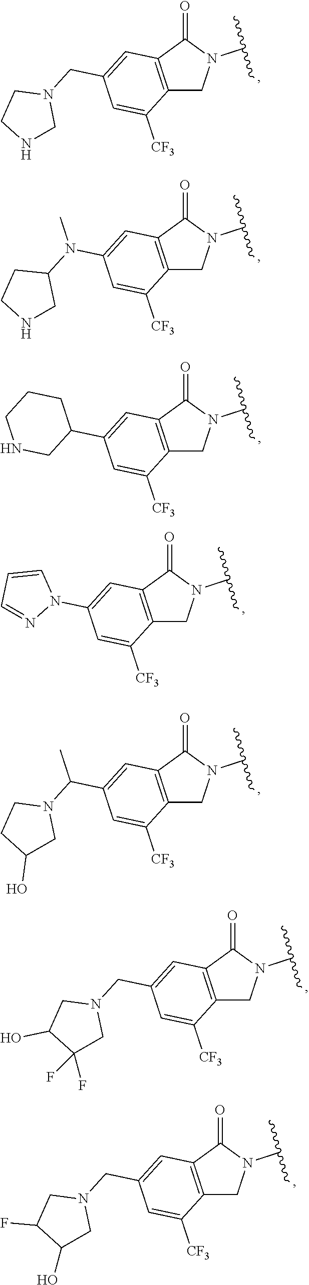

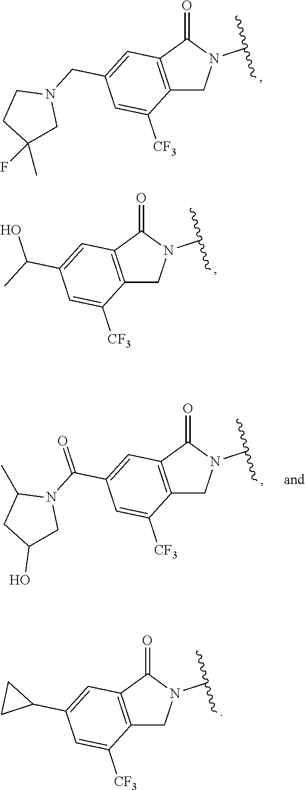 [SMILES: *N1Cc2c(cc(CN3CCC(C)(F)C3)cc2C(F)(F)F)C1=O ; *N1Cc2c(cc(C(C)O)cc2C(F)(F)F)C1=O ; *N1Cc2c(cc(C(=O)N3CC(O)CC3C)cc2C(F)(F)F)C1=O ; *N1Cc2c(cc(C3CC3)cc2C(F)(F)F)C1=O]

In another embodiment, when the compound has a lactam ring, the portion of the compound comprising the lactam ring can be selected from the group consisting of:

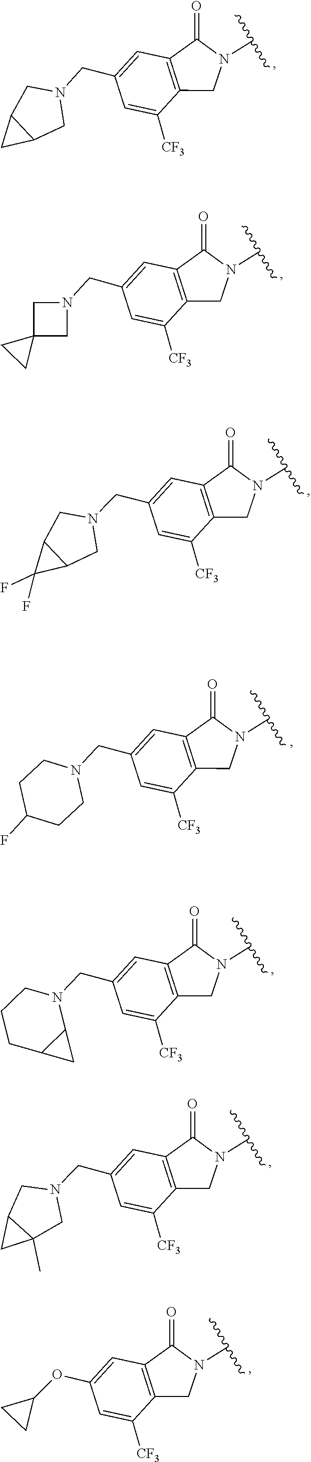

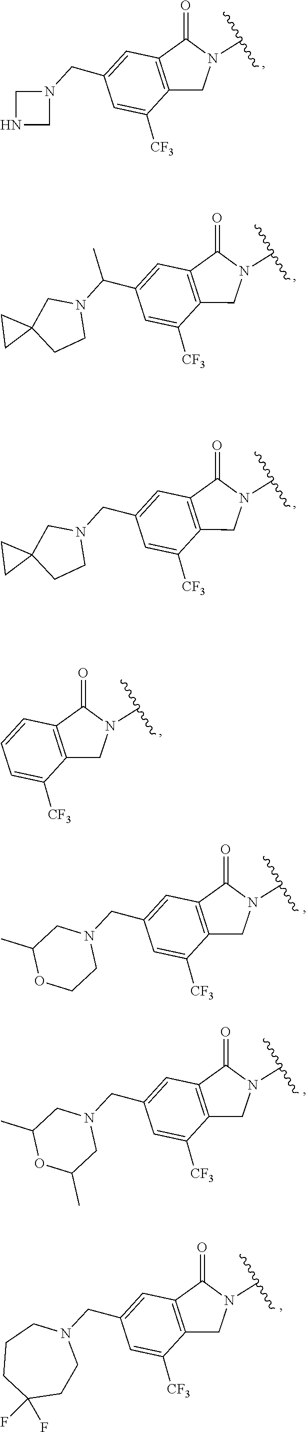

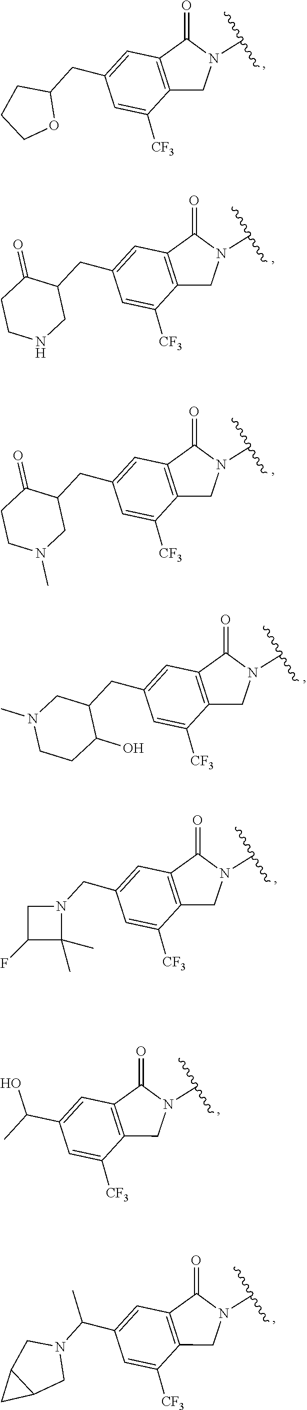

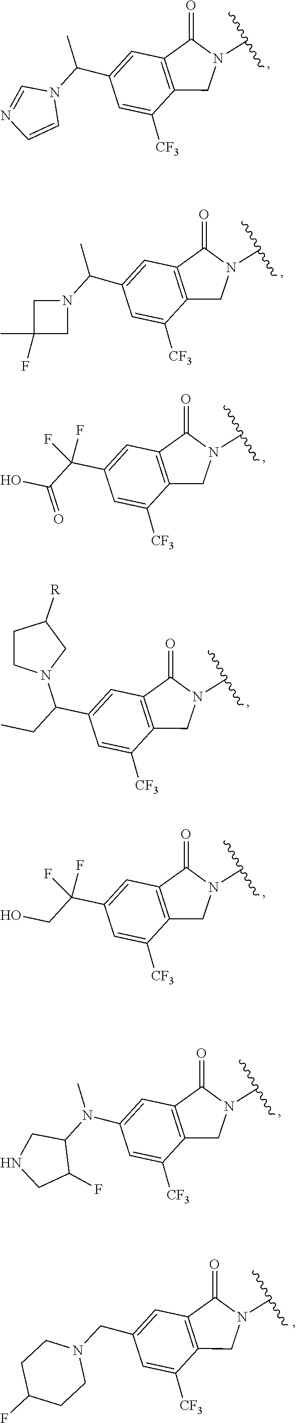

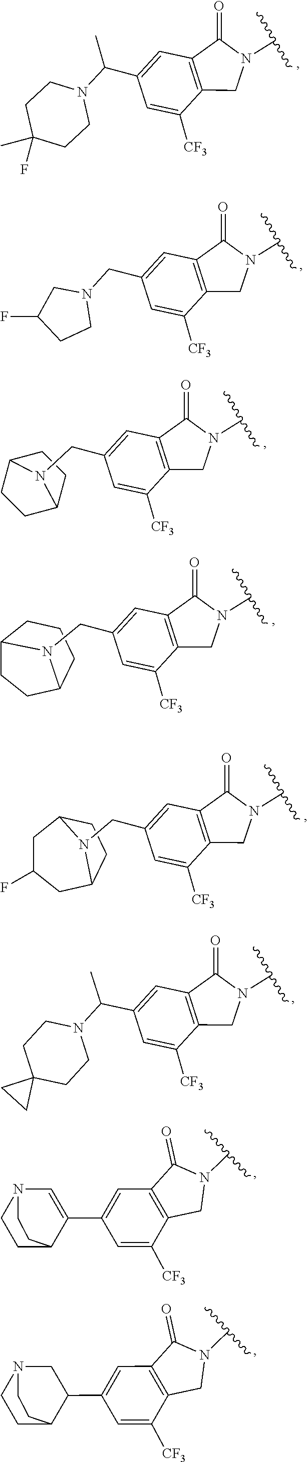

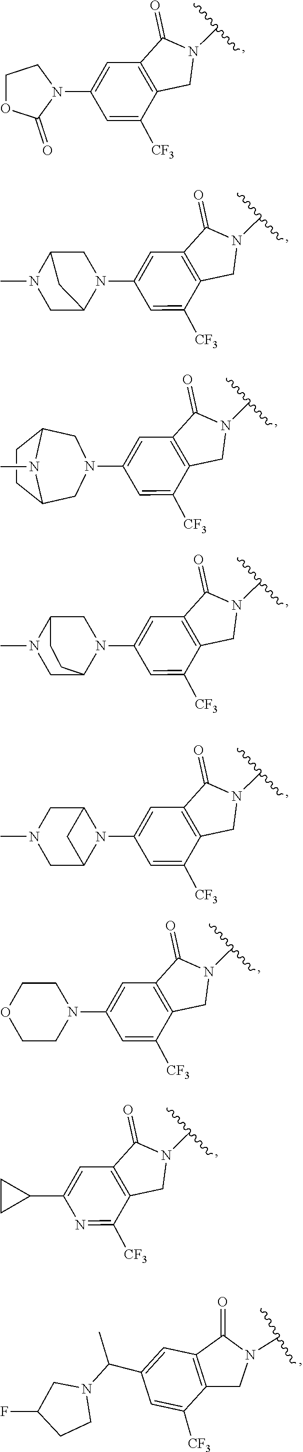

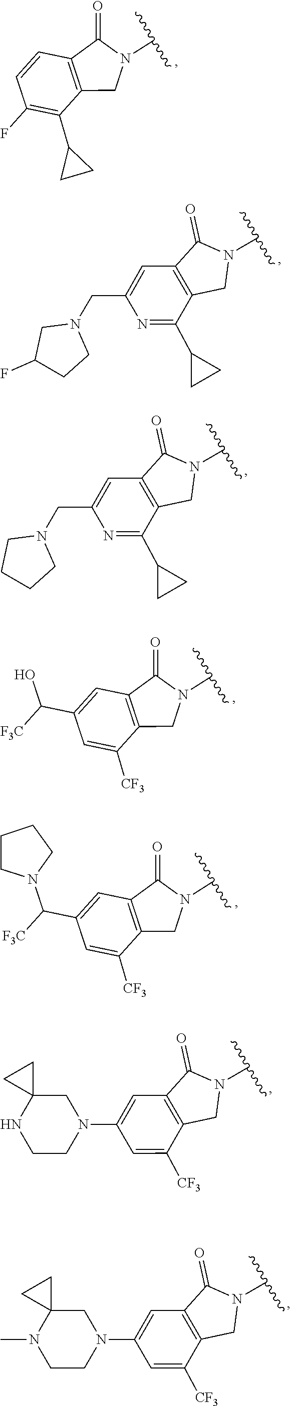

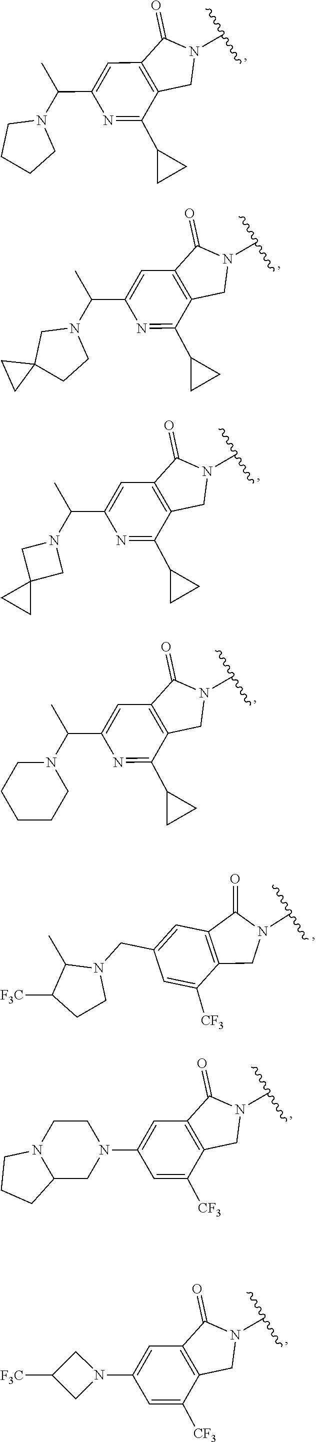

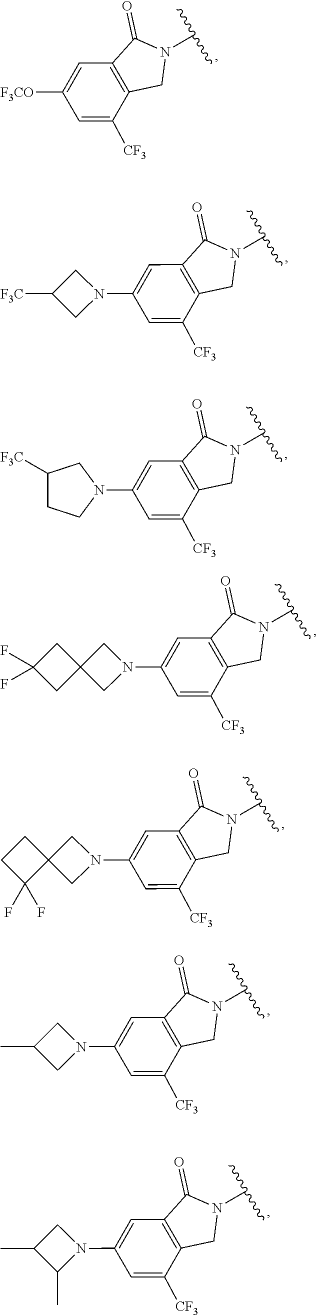

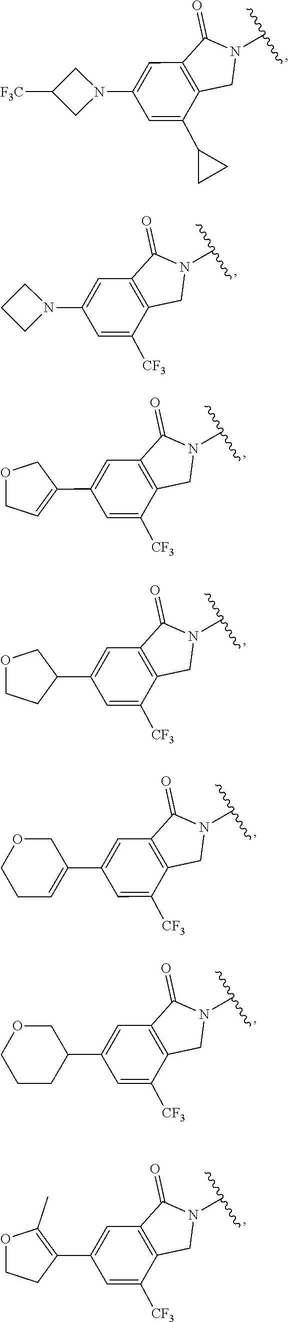

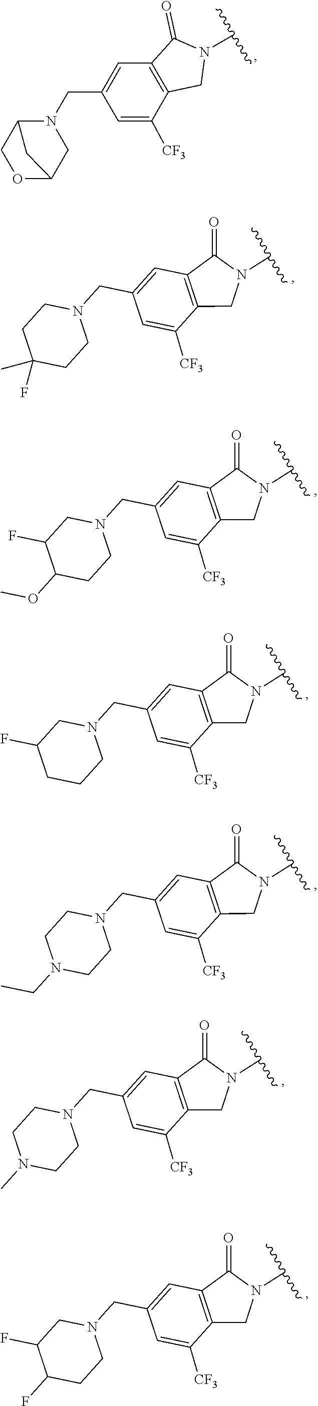

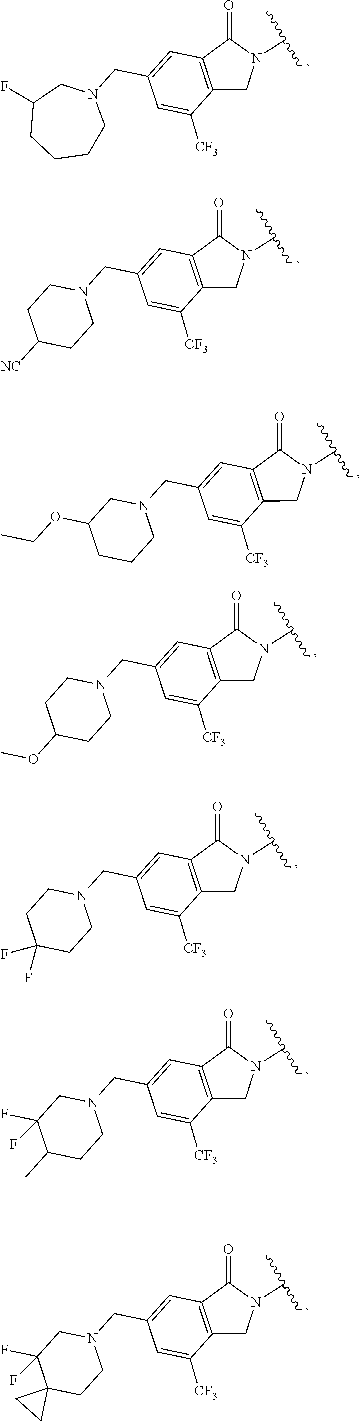

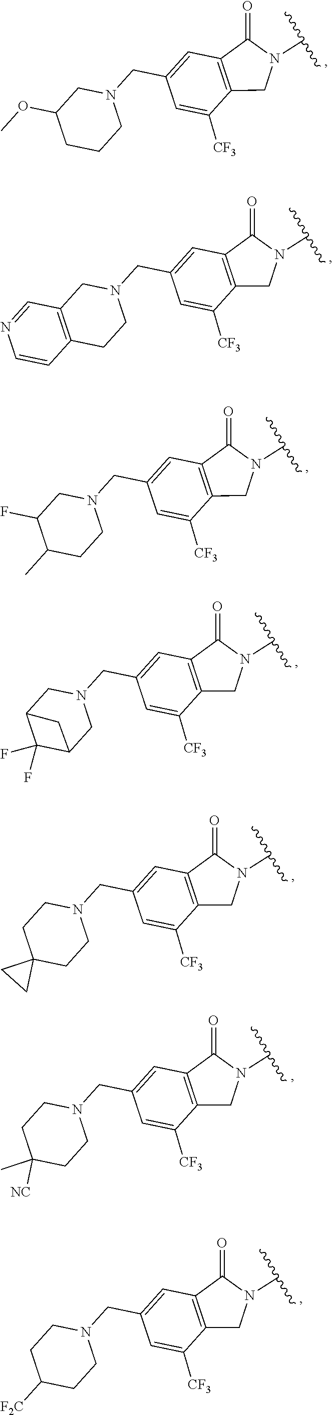

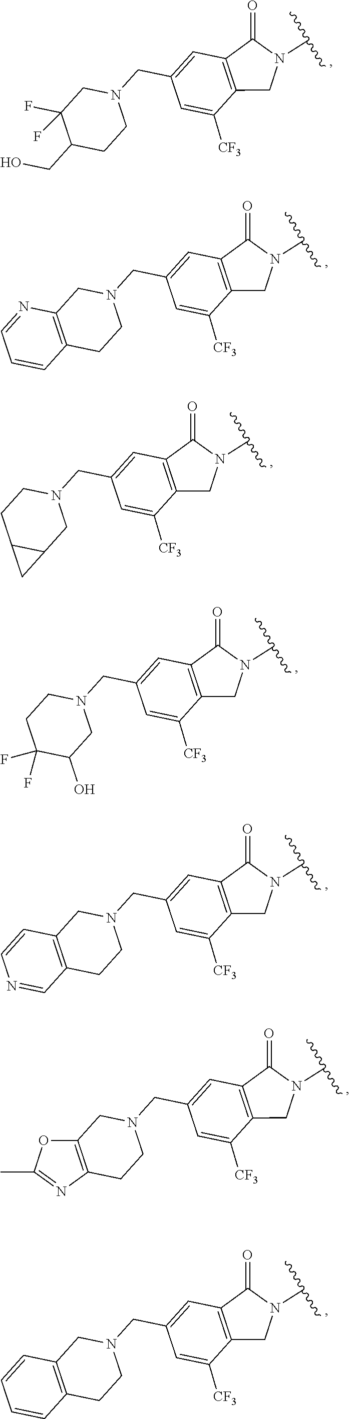

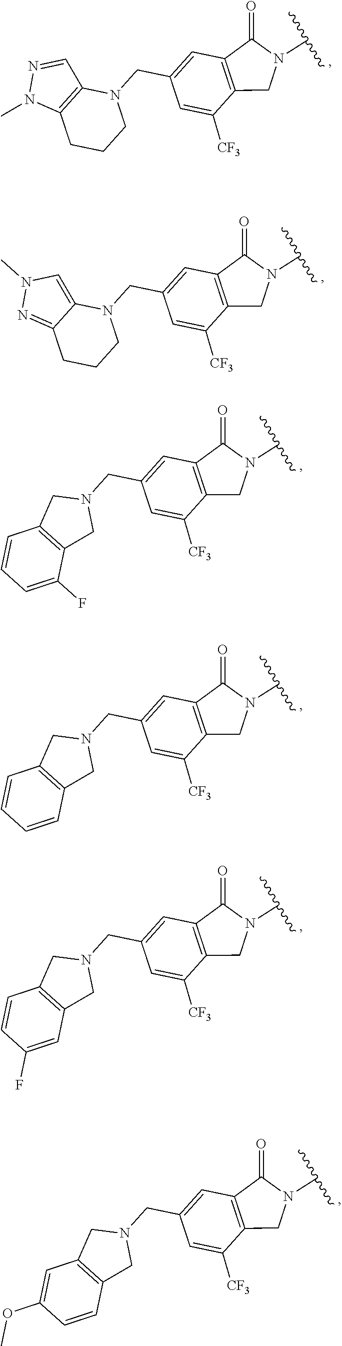 [SMILES: *N1Cc2c(cc(CN3CCCc4c3cnn4C)cc2C(F)(F)F)C1=O ; *N1Cc2c(cc(CN3CCCc4nn(C)cc43)cc2C(F)(F)F)C1=O ; *N1Cc2c(cc(CN3Cc4cccc(F)c4C3)cc2C(F)(F)F)C1=O ; *N1Cc2c(cc(CN3Cc4ccccc4C3)cc2C(F)(F)F)C1=O ; *N1Cc2c(cc(CN3Cc4ccc(F)cc4C3)cc2C(F)(F)F)C1=O ; *N1Cc2c(cc(CN3Cc4ccc(OC)cc4C3)cc2C(F)(F)F)C1=O]

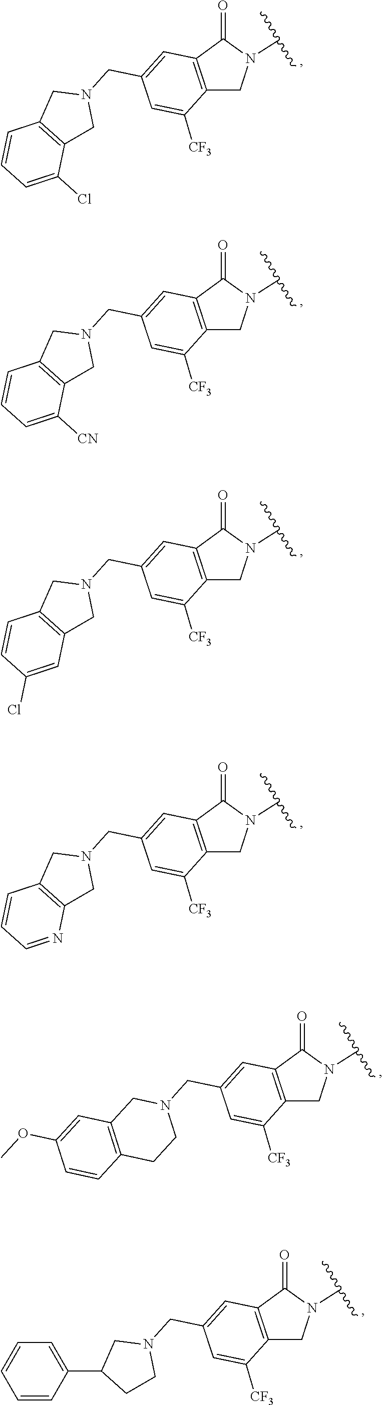 [SMILES: *N1Cc2c(cc(CN3Cc4cccc(Cl)c4C3)cc2C(F)(F)F)C1=O ; *N1Cc2c(cc(CN3Cc4cccc(C#N)c4C3)cc2C(F)(F)F)C1=O ; *N1Cc2c(cc(CN3Cc4ccc(Cl)cc4C3)cc2C(F)(F)F)C1=O ; *N1Cc2c(cc(CN3Cc4cccnc4C3)cc2C(F)(F)F)C1=O ; *N1Cc2c(cc(CN3CCc4ccc(OC)cc4C3)cc2C(F)(F)F)C1=O ; *N1Cc2c(cc(CN3CCC(c4ccccc4)C3)cc2C(F)(F)F)C1=O]

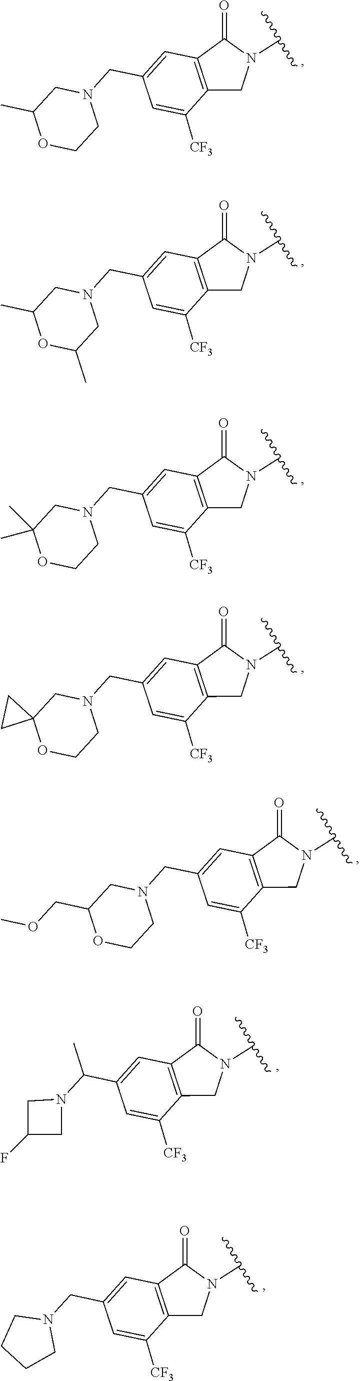

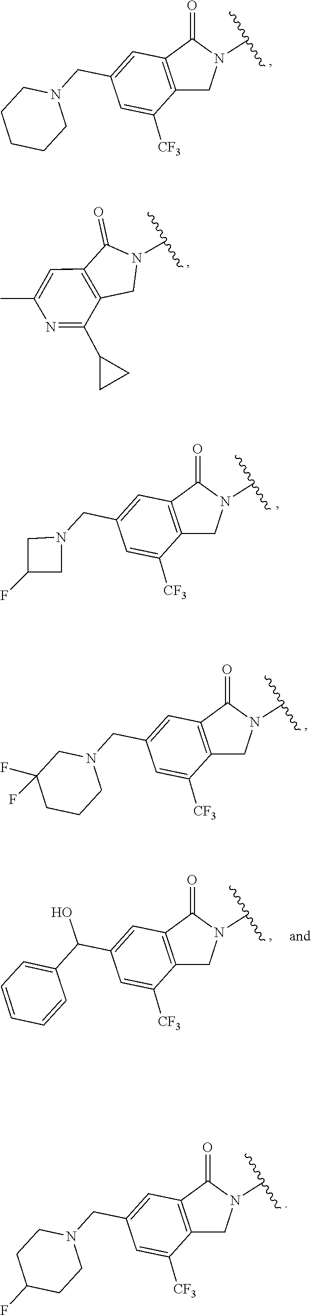 [SMILES: *N1Cc2c(cc(CN3CCCCC3)cc2C(F)(F)F)C1=O ; *N1Cc2c(cc(C)nc2C2CC2)C1=O ; *N1Cc2c(cc(CN3CC(F)C3)cc2C(F)(F)F)C1=O ; *N1Cc2c(cc(CN3CCCC(F)(F)C3)cc2C(F)(F)F)C1=O ; *N1Cc2c(cc(C(O)c3ccccc3)cc2C(F)(F)F)C1=O ; *N1Cc2c(cc(CN3CCC(F)CC3)cc2C(F)(F)F)C1=O]

In one embodiment, when the compound has a lactam ring such as those described in Scheme L, Scheme L′, or Scheme M, the portion of the compound comprising the lactam ring can be

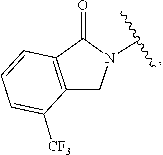 [SMILES: *N1Cc2c(cccc2C(F)(F)F)C1=O]

that is, 4-(trifluoromethyl)isoindoline-1-one attached at the 2-nitrogen to the remainder of the molecule. The 4-(trifluoromethyl)isoindoline-1-one can have additional substituents, particularly at the 6-position, such as the substituents at the 6-position in the structures:

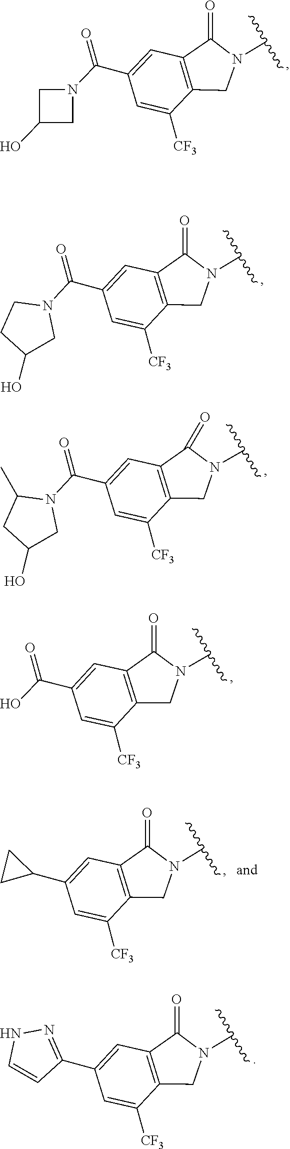 [SMILES: *N1Cc2c(cc(C(=O)N3CC(O)C3)cc2C(F)(F)F)C1=O ; *N1Cc2c(cc(C(=O)N3CCC(O)C3)cc2C(F)(F)F)C1=O ; *N1Cc2c(cc(C(=O)N3CC(O)CC3C)cc2C(F)(F)F)C1=O ; *N1Cc2c(cc(C(=O)O)cc2C(F)(F)F)C1=O ; *N1Cc2c(cc(C3CC3)cc2C(F)(F)F)C1=O ; *N1Cc2c(cc(-c3cc[nH]n3)cc2C(F)(F)F)C1=O]

In another embodiment, when the compound has a lactam ring such as those described in Scheme L, Scheme L′, or Scheme M, the portion of the compound comprising the lactam ring can be

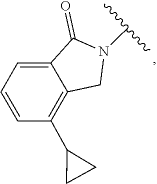 [SMILES: *N1Cc2c(cccc2C2CC2)C1=O]

that is, 4-(cyclopropyl)isoindoline-1-one attached at the 2-nitrogen to the remainder of the molecule. The 4-(cyclopropyl)isoindoline-1-one can have additional substituents, particularly at the 6-position, such as the substituents at the 6-position in the structures:

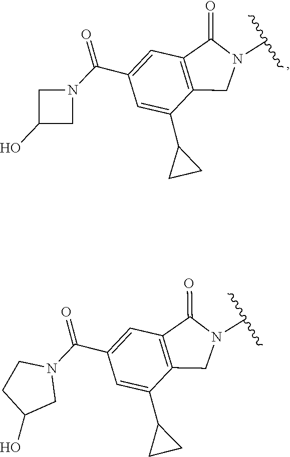 [SMILES: *N1Cc2c(cc(C(=O)N3CC(O)C3)cc2C2CC2)C1=O ; *N1Cc2c(cc(C(=O)N3CCC(O)C3)cc2C2CC2)C1=O]

Particular B rings include 1,2,4-triazole, pyrrole, imidazole, tetrazole, and isoxazole, each of which can be optionally substituted.

Particular linkers between Ring C and Ring B include —CH(CH₃)—CH₂— (in either R or S configuration), —CH(CH₃)—S— (in either R or S configuration), —CH₂—CH₂—, and —CH₂—S—.

Compounds 54, 65, 67, 72, 76, 89, 91, 92, 93, 183a, 191, 193, 194, 195, 196, 199, 203, 205, 209, 211, 220, 255a, 260, 274, 275, 276, 277, 278, 279, 284, 299, 300, and 301 display IC₅₀ values of 0.1 micromolar or less in the Cbl-b inhibition assay of Biological Example 1, and in one embodiment are used for the pharmaceutical compositions and in the methods as disclosed herein.

Compounds 321, 328, 330, 335, 336, 338, 339, 340, 341, 342, 343, 346, 347, 348, 352, 353, 356, 358, 360, 363, 374, 375, 377, 378, 379, 380, 381, 383, 384, 385, 386, 387, 388, 389, 390, 391, 392, 393, 394, 395, 396, 397, 398, 399, 400, 401, 402, 403, 404, 405, 406, 407, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 442, 443, 444, 445, 446, 447, 448, 450, 452, 453, 454, 455, 457, 458, 459, 460, 461, 462, 463, 464, 465, 466, 468, 472, 474, 475, 477, and 479 display IC₅₀ values of 0.1 micromolar or less in the Cbl-b inhibition assay of Biological Example 1, and in one embodiment are used for the pharmaceutical compositions and in the methods as disclosed herein.

Compounds 486a, 486b, 487, 488, 489, 490, 491, 492, 496, 497, 498, 499, 500, 501, 502, 503, 504, 505, 506, 507, 508, 509, 510, 511, 512, 513, 514, 515, 516, 517a, 517b, 519, 520, 521, 524, 525, 526, 527, 528, 529, 530, 531, 532, 533, 534b, 535, 536, 538, 540, 541a, 541b, 542, 543a, 543b, 545, 546, 547, 550, 551, 552, 553a, 553b, 554, 555, 556, 557, 558a, 558b, 560, 561, 562, 563, 564, 565, 566, 567, 568, 569, 570, 571, 572, 573, 574, 575, 576, 578, 579, 580, 581, 582, 583, 584, 585, 586, 587, 588, 589, 590, 591, 592a, 592b, 593, 594, 595, 600, 601, 602, 603, 604, 606, 607, 608, 609, 610, 611, 612, 613, 614, 615, 616, 618, 619, 620, 621, 622, 623, 624, 625, 626a, 626b, 627, 628, 629, 630, 631, 632, 633, 634, 635, 636, 637, 638, 639a, 639b, 640, 641, 642, 643, 644a, 644b, 645, 646, 647, 648, 649, 650, 651, 652, 653, 654, 655, 656, 657, 658, 659, 660, 661, 662a, 662b, 663a, 663b, 664, 665, 666, 667, 668a, 668b, 669a, 669b, 670a, 670b, 671a, 671b, 672a, 672b, 673a, 673b, 674a, 674b, 675a, 675b, 676, 678, 679, 680, 690, 691a, 691b, 692, 693, 694, 695, 696, 697, 698, 699, 702, 703, 704, 706, 707, 708, 709, 710, 711, 712, 713, 714, 715, 716, 717, 718, and 719 also display IC₅₀ values of 0.1 micromolar or less in the Cbl-b inhibition assay of Biological Example 1, and in one embodiment are used for the pharmaceutical compositions and in the methods as disclosed herein.

Compounds 321, 328, 330, 335, 336, 338, 339, 340, 341, 342, 343, 346, 347, 348, 352, 353, 356, 358, 360, 363, 374, 375, 377, 378, 379, 380, 381, 383, 384, 385, 386, 387, 388, 389, 390, 391, 392, 393, 394, 395, 396, 397, 398, 399, 400, 401, 402, 403, 404, 405, 406, 407, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 442, 443, 444, 445, 446, 447, 448, 450, 452, 453, 454, 455, 457, 458, 459, 460, 461, 462, 463, 464, 465, 466, 468, 472, 474, 475, 477, 479, 311, 312a, 313, 319, 320, 323, 325, 326, 329, 332, 333, 334, 337, 344, 345, 351, 354, 357, 362, 365, 366, 367a, 370, 371, 376, 382, 408, 440, 441, 451, 456, 467, 469, 470, 471a, 480, 481, 483, 484, and 485 display IC₅₀ values of 0.3 micromolar or less in the Cbl-b inhibition assay of Biological Example 1, and in one embodiment are used for the pharmaceutical compositions and in the methods as disclosed herein.

Compounds 486a, 486b, 487, 488, 489, 490, 491, 492, 496, 497, 498, 499, 500, 501, 502, 503, 504, 505, 506, 507, 508, 509, 510, 511, 512, 513, 514, 515, 516, 517a, 517b, 519, 520, 521, 524, 525, 526, 527, 528, 529, 530, 531, 532, 533, 534b, 535, 536, 538, 540, 541a, 541b, 542, 543a, 543b, 545, 546, 547, 550, 551, 552, 553a, 553b, 554, 555, 556, 557, 558a, 558b, 560, 561, 562, 563, 564, 565, 566, 567, 568, 569, 570, 571, 572, 573, 574, 575, 576, 578, 579, 580, 581, 582, 583, 584, 585, 586, 587, 588, 589, 590, 591, 592a, 592b, 593, 594, 595, 600, 601, 602, 603, 604, 606, 607, 608, 609, 610, 611, 612, 613, 614, 615, 616, 618, 619, 620, 621, 622, 623, 624, 625, 626a, 626b, 627, 628, 629, 630, 631, 632, 633, 634, 635, 636, 637, 638, 639a, 639b, 640, 641, 642, 643, 644a, 644b, 645, 646, 647, 648, 649, 650, 651, 652, 653, 654, 655, 656, 657, 658, 659, 660, 661, 662a, 662b, 663a, 663b, 664, 665, 666, 667, 668a, 668b, 669a, 669b, 670a, 670b, 671a, 671b, 672a, 672b, 673a, 673b, 674a, 674b, 675a, 675b, 676, 678, 679, 680, 690, 691a, 691b, 692, 693, 694, 695, 696, 697, 698, 699, 702, 703, 704, 706, 707, 708, 709, 710, 711, 712, 713, 714, 715, 716, 717, 718, 719, 494, 518, 522, 523, 534a, 548, 549, 559, 577, 596, 597, 598, 605, 617, 683, and 700 display IC₅₀ values of 0.3 micromolar or less in the Cbl-b inhibition assay of Biological Example 1, and in one embodiment are used for the pharmaceutical compositions and in the methods as disclosed herein.

Compounds 8a, 9a, 30, 33, 34, 39, 48, 51, 54, 57a, 61, 62, 65, 67, 68, 69, 70, 71, 72, 75, 76, 77, 78, 81, 85, 89, 91, 92, 93, 95a, 120, 140, 148, 166a, 167, 168a, 168, 182, 183a, 186, 187, 188, 189, 190, 191, 193, 194, 195, 196, 198, 199, 200, 201, 203, 205, 209, 211, 212, 213a, 218, 220, 224, 240, 243, 255a, 256, 257, 258, 260, 262, 268, 272b, 272, 274, 275, 276, 277, 278, 279, 281a, 281, 282, 283b, 284, 285, 286, 287, 289, 293, 294, 296a, 298b, 298, 299, 300, 301, and 304b display IC₅₀ values of 0.5 micromolar or less in the Cbl-b inhibition assay of Biological Example 1, and in one embodiment are used for the pharmaceutical compositions and in the methods as disclosed herein.

Compounds 8a, 9a, 9, 30, 33, 34, 35, 38, 39, 42, 48, 51, 54, 55, 56, 57a, 61, 62, 64, 65, 67, 68, 69, 70, 71, 72, 74, 75, 76, 77, 78, 81, 84, 85, 89, 91, 92, 93, 95a, 98, 99, 103, 104, 105, 110, 112, 113, 119, 120, 130a, 140, 148, 151, 164, 166a, 167, 168a, 168, 172a, 176, 178, 182, 183a, 185, 186, 187, 188, 189, 190, 191, 192, 193, 194, 195, 196, 198, 199, 200, 201, 202, 203, 205, 206, 207, 208, 209, 211, 212, 213a, 215, 218, 220, 221, 222, 224, 240, 243, 251, 255a, 256, 257, 258, 259, 260, 262, 265, 268, 270a, 272b, 272, 274, 275, 276, 277, 278, 279, 281a, 281, 282, 283b, 283, 284, 285, 286, 287, 289, 293, 294, 296a, 296, 298b, 298, 299, 300, 301, 302, 303, and 304b display IC₅₀ values of 1 micromolar or less in the Cbl-b inhibition assay of Biological Example 1, and in one embodiment are used for the pharmaceutical compositions and in the methods as disclosed herein.

Compounds 493, 495, 544, 599, 677, 681, 682, 684, 685, 686, 687, 688, 689a, 689b, and 701 display IC₅₀ values of 1 micromolar or less in the Cbl-b inhibition assay of Biological Example 1, and in one embodiment are used for the pharmaceutical compositions and in the methods as disclosed herein.

Compounds 8a, 8, 8b, 9a, 9, 23, 26, 30, 32, 33, 34, 35, 38, 39, 40, 41, 42, 43, 44, 45, 46, 47, 48, 49, 50, 51, 52, 53, 54, 55, 56, 57a, 57b, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95a, 95, 95b, 96a, 97, 98, 99, 102b, 103, 104, 105, 106, 107, 108, 109, 110, 111, 112, 113, 114, 115, 116, 117, 118, 119, 120, 121, 122, 123, 124, 125, 126, 127, 128a, 129, 130a, 131, 134, 135, 138, 139, 140, 141, 142, 143, 144, 145, 146, 147, 148, 149, 150, 151, 152, 153, 154, 155, 156, 157, 158, 159, 160, 161, 163, 164, 165, 166a, 166b, 167, 168a, 168, 168b, 169, 172a, 172b, 173, 174, 175, 176, 177, 178, 179, 180, 181, 182, 183a, 183b, 184, 185, 186, 187, 188, 189, 190, 191, 192, 193, 194, 195, 196, 197, 198, 198b, 199, 200, 201, 202, 203, 204, 205, 206, 207, 208, 209, 210, 211, 212, 213a, 213b, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252a, 252, 253, 254, 255a, 255b, 256, 257, 258, 259, 260, 261, 262, 263, 264, 265, 266, 267a, 267, 268, 269, 270a, 271b, 271, 272b, 272, 273, 274, 275, 276, 277, 278, 279, 280, 281a, 281, 281b, 282, 283b, 283, 284, 285, 286, 287, 288, 289, 290, 291, 292, 293, 294, 295, 296a, 296, 296b, 297, 298b, 298, 298a, 299, 300, 301, 302, 303, 304b, and 304a display IC₅₀ values of 10 micromolar or less in the Cbl-b inhibition assay of Biological Example 1, and in one embodiment are used for the pharmaceutical compositions and in the methods as disclosed herein.

Compounds 1, 2, 3, 4, 5, 6, 7, 9b, 10, 11, 12, 13, 14, 15, 16, 17, 18, 19, 20, 21, 22, 24, 25, 27, 28, 29, 31, 36, 37, 96, 100, 102a, 132, 133, 136, 137, 162, 166, 171 were tested using an alternate version of the Cbl-b inhibition assay, as described in Biological Example 1, and in one embodiment are used for the pharmaceutical compositions and in the methods as disclosed herein.

In one embodiment, compounds 8a, 57a, 140, 255a, 183a, 282, and 187 are used for the pharmaceutical compositions and in the methods as disclosed herein.

In various embodiments, and as further described herein, compounds as provided herein (as well as compositions comprising compounds described herein, and methods using the compounds or compositions) have IC₅₀ values of less than 100 nM, between 100 nM-300 nM, between 301 nM-1000 nM, between 1,001 nM-3,000 nM, between 3,001 nM-10,000 nM, or greater than 10,000 nM. In a further embodiment, and as further described herein, compounds as provided herein (as well as compositions comprising compounds described herein, and methods using the compounds or compositions) have IC₅₀ values of less than 100 nM. In a further embodiment, and as further described herein, compounds as provided herein (as well as compositions comprising compounds described herein, and methods using the compounds or compositions) have IC₅₀ values of between 100 nM-300 nM. In a further embodiment, and as further described herein, compounds as provided herein (as well as compositions comprising compounds described herein, and methods using the compounds or compositions) have IC₅₀ values of between 301 nM-1000 nM. In a further embodiment, and as further described herein, compounds as provided herein (as well as compositions comprising compounds described herein, and methods using the compounds or compositions) have IC₅₀ values of between 1,001 nM-3,000 nM. In a further embodiment, and as further described herein, compounds as provided herein (as well as compositions comprising compounds described herein, and methods using the compounds or compositions) have IC₅₀ values of between 3,001 nM-10,000 nM. In a further embodiment, and as further described herein, compounds as provided herein (as well as compositions comprising compounds described herein, and methods using the compounds or compositions) have IC₅₀ values of greater than 10,000 nM.

For IL-2 secretion from an immune cell (e.g., T-cell) co-stimulated with an anti-CD3 antibody and an anti-CD28 antibody, compounds as provided herein (as well as compositions comprising compounds described herein) induce ≤1.00 fold, between 1.01-2.50 fold, between 2.51-5.00 fold, between 5.00-7.50 fold, or ≥7.50 fold change over baseline, at inhibitor concentrations of 4 micromolar, 1 micromolar, or 0.3 micromolar.

For IL-2 secretion from an immune cell (e.g., T-cell) stimulated with an anti-CD3 antibody, compounds as provided herein (as well as compositions comprising compounds described herein) induce ≤0.050 fold, between 0.051-0.1 fold, between 0.101-0.15 fold, between 0.151-0.2 fold, or ≥0.201 fold change over baseline, at inhibitor concentrations of 8 micromolar or 1 micromolar.

For CD25 staining on the cell surface of an immune cell (e.g., T-cell) co-stimulated with an anti-CD3 antibody and an anti-CD28 antibody, compounds as provided herein (as well as compositions comprising compounds described herein) induce ≤1.00 fold, between 1.01-1.30 fold, between 1.31-1.50 fold, between 1.51-1.75 fold, or ≥1.76 fold change over baseline, at inhibitor concentrations of 4 micromolar, 1 micromolar, or 0.3 micromolar.

For CD25 staining on the cell surface of an immune cell (e.g., T-cell) stimulated with an anti-CD3 antibody, compounds as provided herein (as well as compositions comprising compounds described herein) induce ≤0.600 fold, between 0.601<0.800 fold, between 0.801<1.00 fold, or ≥1.001 fold change over baseline, at inhibitor concentrations of 8 micromolar or 1 micromolar.

In some embodiments, the compounds listed in Table IX, tautomers and stereoisomers thereof, and salts of any of the foregoing, are excluded from the general disclosure. In some embodiments, the compounds listed in Table IX, tautomers and stereoisomers thereof, and salts of any of the foregoing, are excluded from the compounds used in any of the pharmaceutical compositions disclosed herein. In some embodiments, the compounds listed in Table IX, tautomers and stereoisomers thereof, and salts of any of the foregoing, are excluded from the compounds used in any of the methods disclosed herein.

TABLE 1X

| “X” number | Structure and Name |
|---|---|
| 1X | 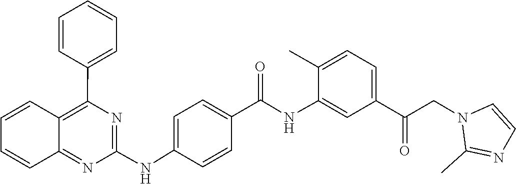 [SMILES: Cc1ccc(C(=O)Cn2ccnc2C)cc1NC(=O)c1ccc(Nc2nc(-c3ccccc3)c3ccccc3n2)cc1] |
| | N-[2-methyl-5-[2-(2-methyl-1H-imidazol-1-yl)acetyl]phenyl]-4-[(4- |
| | phenyl-2-quinazolinyl)amino]-benzamide |
| 2X | 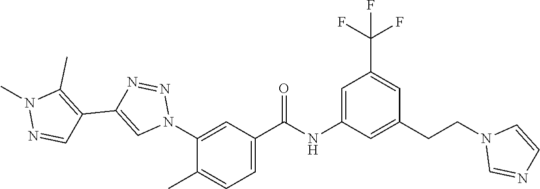 [SMILES: Cc1ccc(C(=O)Nc2cc(CCn3ccnc3)cc(C(F)(F)F)c2)cc1-n1cc(-c2cnn(C)c2C)nn1] |
| | 3-[4-(1,5-dimethyl-1H-pyrazol-4-yl)-1H-1,2,3-triazol-1-yl]-N-[3-[2-(1H- |
| | imidazol-1-yl)ethyl]-5-(trifluoromethyl)phenyl]-4-methyl-benzamide |
| 3X | 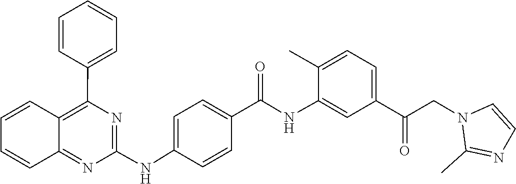 [SMILES: Cc1ccc(C(=O)Cn2ccnc2C)cc1NC(=O)c1ccc(Nc2nc(-c3ccccc3)c3ccccc3n2)cc1] |
| | N-[2-methyl-5-[2-(2-methyl-1H-imidazol-1-yl)acetyl]phenyl]-4-[(4- |
| | phenyl-2-quinazolinyl)amino]-benzamide |
| 4X | 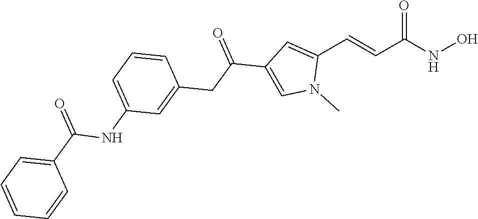 [SMILES: Cn1cc(C(=O)Cc2cccc(NC(=O)c3ccccc3)c2)cc1/C=C/C(=O)NO] |
| | N-[3-[2-[5-[3-(hydroxyamino)-3-oxo-1-propen-1-yl]-1-methyl-1H-pyrrol- |
| | 3-yl]-2-oxoethyl]phenyl]-benzamide |
| 5X | 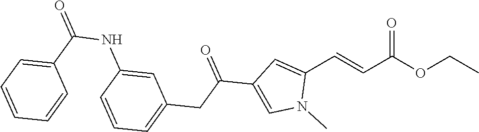 [SMILES: CCOC(=O)/C=C/c1cc(C(=O)Cc2cccc(NC(=O)c3ccccc3)c2)cn1C] |
| | 3-[4-[2-[3-(benzoylamino)phenyl]acetyl]-1-methyl-1H-pyrrol-2-yl]- 2- |
| | propenoic acid,, ethyl ester |
| 6X | 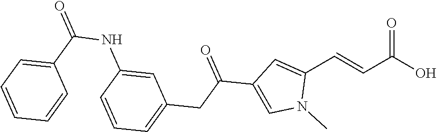 [SMILES: Cn1cc(C(=O)Cc2cccc(NC(=O)c3ccccc3)c2)cc1/C=C/C(=O)O] |
| | 3-[4-[2-[3-(benzoylamino)phenyl]acetyl]-1-methyl-1H-pyrrol-2-yl]-2- |
| | propenoic acid, |
| 7X | 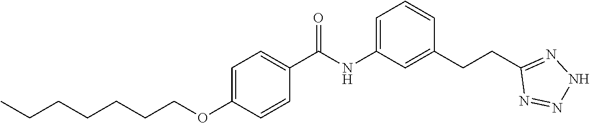 [SMILES: CCCCCCCOc1ccc(C(=O)Nc2cccc(CCc3nn[nH]n3)c2)cc1] |
| | 4-(heptyloxy)-N-[3-[2-(2H-tetrazol-5-yl)ethyl]phenyl]-benzamide |
| 8X | 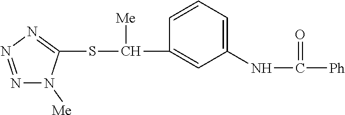 [SMILES: CC(Sc1nnnn1C)c1cccc(NC(=O)c2ccccc2)c1] |
| 9X | 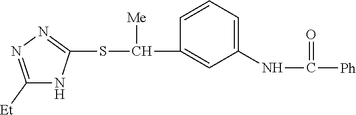 [SMILES: CCc1nnc(SC(C)c2cccc(NC(=O)c3ccccc3)c2)[nH]1] |
| 10X | 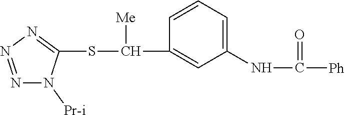 |
| 11X | 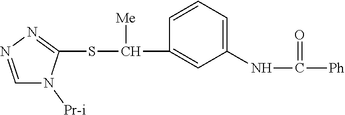 |
| 12X | 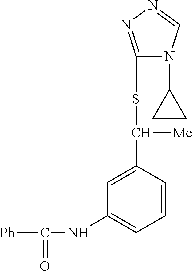 [SMILES: CC(Sc1nncn1C1CC1)c1cccc(NC(=O)c2ccccc2)c1] |
| 13X | 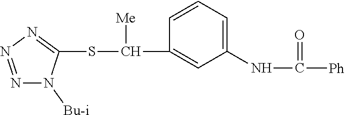 |
| 14X | 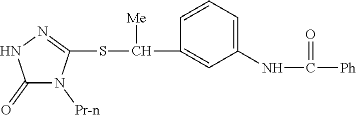 |
| 15X | 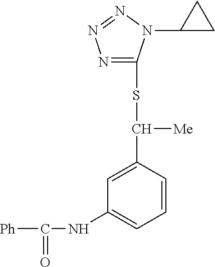 [SMILES: CC(Sc1nnnn1C1CC1)c1cccc(NC(=O)c2ccccc2)c1] |
| 16X | 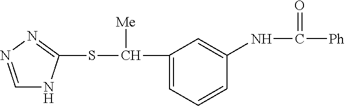 [SMILES: CC(Sc1nnc[nH]1)c1cccc(NC(=O)c2ccccc2)c1] |
| 17X | 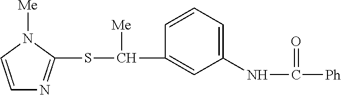 [SMILES: CC(Sc1nccn1C)c1cccc(NC(=O)c2ccccc2)c1] |
| 18X | 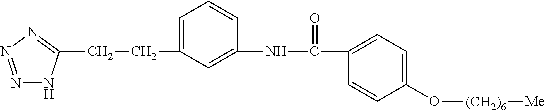 |
| 19X | 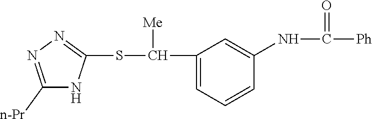 |
| 20X | 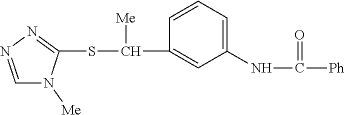 [SMILES: CC(Sc1nncn1C)c1cccc(NC(=O)c2ccccc2)c1] |

The chemical structures in the table above were generated from Chemical Abstracts REGISTRY chemical names using ChemDraw® Professional version 16.0.0.82 (68) software.

### III. Use and Methods

Provided herein are methods for modulating activity of an immune cell (e.g., a T-cell, a B cell or a NK cell) such as by contacting the immune cell with an effective amount of a Cbl-b inhibitor described herein or a composition thereof. Also provided are in vitro methods of producing said immune cells with modulated activity, referred to herein as “modified immune cells,” wherein said modified immune cells can be administered to an individual in need thereof (e.g., an individual having cancer) by ex vivo methods. Also provided herein are in vivo methods of modulating a response in an individual in need thereof (e.g., an individual with cancer), wherein the method comprises administration of an effective amount of a Cbl-b inhibitor described herein or a composition thereof.

Additionally, provided are Cbl-b inhibitors for use as therapeutic active substances. A Cbl-b inhibitor for use in treating or preventing a disease or condition associated with Cbl-b activity is provided. Also, a Cbl-b inhibitor for use in treating cancer is provided. Further provided is the use of a Cbl-b inhibitor in the manufacture of a medicament for treating or preventing a disease or condition associated with Cbl-b activity. Also provided is the use of a Cbl-b inhibitor in the manufacture of a medicament for treating cancer.

A. Isolation and Processing of Cells

Provided are methods for the preparation and processing of immune cells produced (e.g., modified immune cells) and used in the methods herein. As used herein, the term “modified immune cells” refers to immune cells or a cell population comprising the immune cells which have been cultured, incubated and/or have been contacted with an effective amount of a Cbl-b inhibitor to modulate the activity of said immune cells. In some embodiments, the modified immune cells can be used for immunotherapy, such as in connection with adoptive immunotherapy methods.

Samples

In some embodiments, the immune cells to be modified or cell populations comprising the immune cells to be modified are isolated from a sample, such as a biological sample, e.g., one obtained from or derived from an individual (e.g., a human). In some embodiments, the individual from which the immune cell is isolated is one having a particular disease or condition (e.g., cancer) or in need of a cell therapy or to which cell therapy will be administered. The individual in some embodiments is a human in need of a particular therapeutic intervention, such as the adoptive cell therapy for which immune cells are being isolated, processed, and/or modified. Accordingly, the cells isolated from the individual in some embodiments are primary cells (e.g., primary human cells). As used herein, the term “primary cells” refers to cells isolated directly from mammal biological fluid or tissue (e.g., human biological fluid or tissue).

In some embodiments, the immune cells to be modified are hematopoietic cells, multipotent stem cells, myeloid progenitor cells, lymphoid progenitor cells, T-cells, B cells, and/or NK cells. As used herein, the term “hematopoietic cells” includes hematopoietic stem cells and hematopoietic progenitor cells. In some embodiments, the immune cells to be modified are present in a heterogeneous cell population or a composition comprising a heterogeneous cell population. For example, the immune cells to be modified may be hematopoietic cells present in a heterogeneous cell population comprising cells such as differentiated cells derived from a tissue or organ. In some embodiments, the immune cells to be modified are present in a homogenous cell population or a composition comprising a homogenous cell population. For example, the immune cells to be modified may be hematopoietic cells present in a homogenous cell population comprising only hematopoietic cells. In some embodiments, the immune cells to be modified or cell populations comprising the immune cells to be modified include one or more subsets of immune cells. For example, one or more subsets of T cells or other cell types, such as whole T cell populations, CD4+ cells, CD8+ cells and subpopulations thereof, such as those defined by function, activation state, maturity, potential for differentiation, expansion, localization, persistence capacities, surface marker profile, cytokine secretion profile, and/or degree of differentiation.

In some embodiments, biological samples described herein includes tissue, fluid, and other samples taken directly from the individual, as well as samples resulting from one or more processing steps, such as separation, centrifugation, genetic engineering (e.g., transduction with a viral vector encoding a recombinant chimeric receptor), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples (e.g., sample from a tissue or organ containing a tumor), including processed samples derived therefrom. In some embodiments, the biological sample is a biological fluid sample or a biological tissue sample. In some embodiments, the biological sample is a biological tissue sample.

In some aspects, the biological sample from which the immune cells are derived or isolated is blood or a blood-derived sample, or is derived from an apheresis or leukapheresis product.

Exemplary biological samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Biological samples include, in the context of cell therapy (e.g., adoptive cell therapy) samples from autologous sources (i.e., obtained from or derived from the individual in need of cell therapy) and allogeneic sources (i.e., obtained from or derived from an individual or source other than the individual in need of cell therapy).

In some embodiments, the immune cells to be modified or a cell population comprising the immune cells to be modified are derived from a cell line (e.g., a T-cell line, a B cell line, a NK cell line, etc.). In some embodiments, the immune cells to be modified or a cell population comprising the immune cells to be modified are obtained from a xenogeneic source, such as from mouse, rat, non-human primate, or pig.

Cell Processing and Separation

In some embodiments, isolation of the immune cells to be modified includes one or more preparation and/or cell separation steps. The one or more cell separation steps can be non-affinity based separation or affinity based separation. As an example, non-affinity based separation can be centrifugation of a composition comprising the immune cells to be modified. In some embodiments, the non-affinity based separation methods include density-based cell separation methods, such as the preparation of white blood cells from peripheral blood by lysing the red blood cells and centrifugation through a Percoll or Ficoll gradient. Affinity based separation methods can include contacting a composition comprising the immune cells to be modified with antibody coated beads. Antibody coated beads contemplated herein include, but are not limited to, magnetic beads (e.g., Dynabeads® or MACS® microbeads) coated with an antibody that binds to a marker expressed on the surface of the immune cell to be modified. In some embodiments, specific subpopulations of T cells, such as cells positive or expressing high levels of one or more surface markers, e.g., CD4+, CD8+, etc., are isolated by positive or negative selection techniques. Positive selection can be based on a technique in which the target cells (e.g., immune cells to be modified) have bound to a reagent and are retained for further use. For example, T-cells that are CD3+ can be positively selected using magnetic beads conjugated to anti-CD3 antibodies (e.g., MACS® CD3 human microbeads). Negative selection can be based on a technique in which the targets cells (e.g., immune cells to be modified) that have not bound to a reagent are retained. For example, total human primary T-cells can be isolated from peripheral blood mononuclear cells (PMBCs) utilizing negative selection, wherein a cocktail of antibodies against surface markers CD14, CD15, CD16, CD19, CD34, CD36, CD56, CD123 and CD235a are incubated in a sample comprising the PBMCs before passing the sample by magnetic beads for removal of cells expressing those surface markers and retaining the remaining cells in the sample for subsequent processing. In some embodiments, the immune cells or a cell population comprising the immune cells to be modified are washed, centrifuged, and/or incubated in the presence of one or more reagents, for example, to remove unwanted components, enrich for desired components, lyse or remove cells sensitive to particular reagents. In some examples, the immune cells are separated based on one or more property, such as density, adherent properties, size, sensitivity and/or resistance to particular components. Cell separation steps do not require 100% enrichment or removal of particular cells. In some embodiments, positive selection of or enrichment for immune cells of a particular type (e.g., CD4+ T cells) refers to increasing the number or percentage of such cells. In some embodiments, removal, or depletion of cells of a particular type that are not of interest such as by negative selection, refers to decreasing the number or percentage of such cells.

In some embodiments, immune cells or a cell population comprising the immune cells are obtained from the circulating blood of an individual, e.g., by apheresis or leukapheresis. In some aspects, a sample comprising the immune cells to be modified contain lymphocytes, including T-cells, monocytes, granulocytes, B cells, other nucleated white blood cells, red blood cells, and/or platelets, and in some aspects contains cells other than red blood cells and platelets.

In some embodiments, the blood cells collected from the individual are washed such as to remove the plasma fraction and to place the cell population comprising the immune cells to be modified in an appropriate buffer or media for subsequent processing steps. In some embodiments, the cell population comprising the immune cells to be modified is washed with phosphate buffered saline. In some embodiments, the wash solution lacks calcium and/or magnesium. In some aspects, a washing step is accomplished by a semi-automated “flow-through” centrifuge. In some aspects, a washing step is accomplished by tangential flow filtration. In some embodiments, the immune cells to be modified or cell population containing the immune cells to be modified are resuspended in a variety of suitable buffers after washing, such as, for example, calcium and/or magnesium free phosphate buffered saline. In some embodiments, components of a blood cell sample are removed and the immune cells to be modified or a cell population comprising the immune cells to be modified are directly resuspended in a suitable cell culture medium.

Representative methods for processing and/or separation of immune cells such as hematopoietic cells from samples containing a cell population containing said hematopoietic cells (e.g., samples comprising peripheral blood mononuclear cells (PBMCs)) are described in Biological Example 2 and Biological Example 3 herein. Methods and techniques for the processing and/or separation of immune cells such as hematopoietic cells, multipotent stem cells, myeloid progenitor cells, lymphoid progenitor cells, T-cells, B cells, and/or NK cells are well known in the art. See for example, U.S. Patent Application No. 2017/0037369; U.S. Patent Application No. 2012/0148553; U.S. Pat. Nos. 6,461,645; 6,352,694; and 7,776,562.

Incubation and Treatment

Provided herein are methods for modulating the activity of an immune cell, such as the processed and/or separated immune cells described above by contacting the immune cell with an effective amount of a Cbl-b inhibitor described herein. Also provided herein are modified immune cells produced by any of the methods described herein such as by culturing a cell population containing an immune cell (e.g., the processed and/or separated immune cells described above) in the presence of an effective amount of a Cbl-b inhibitor to modulate the activity of the immune cell and thereby produce the modified immune cell.

In some embodiments, the immune cells to be modified (e.g., the processed and/or separated immune cells described above) are incubated and/or cultured in a suitable culture medium prior to contacting said immune cells with a Cbl-b inhibitor provided herein. In some embodiments, the immune cells to be modified are incubated and/or cultured in a suitable culture medium simultaneously to contacting said immune cells with a Cbl-b inhibitor provided herein.

The processed and/or separated immune cells to be modified or cell population comprising the immune cells to be modified can be differentiated and/or expanded in vitro. In some embodiments, the immune cells to be modified are hematopoietic cells, multipotent stem cells, myeloid progenitor cells, lymphoid progenitor cells, T-cells, B cells, and/or NK cells. In some embodiments, the immune cell to be modified is incubated in a suitable cell culture medium comprising a Cbl-b inhibitor described herein before differentiation and/or expansion of the immune cell. In some embodiments, the immune cell to be modified is incubated in a suitable cell culture medium comprising a Cbl-b inhibitor described herein after differentiation and/or expansion of the immune cell. The immune cells become modified (i.e., modified immune cells) upon contact with a Cbl-b inhibitor provided herein in an effective amount to modulate the activity of said immune cells. In some embodiments, the immune cell to be modified is not differentiated and/or expanded in vitro and is therefore the same cell type as the modified immune cell that has been contacted with a Cbl-b inhibitor. For example, a T-cell can be incubated in a suitable medium comprising a Cbl-b inhibitor without differentiation of the T-cell. In some embodiments, the immune cell to be modified is differentiated and/or expanded in vitro and is therefore a different cell type as the modified immune cell that has been contacted with a Cbl-b inhibitor. For example, a hematopoietic cell can be incubated in a suitable medium comprising a Cbl-b inhibitor as well as other agents that drive differentiation of the hematopoietic cell into a T-cell. Accordingly, in some aspects of the embodiments herein, the modified immune cells are hematopoietic cells, multipotent stem cells, myeloid progenitor cells, lymphoid progenitor cells, T-cells, B cells, and/or NK cells. Methods for expansion and/or differentiation of immune cells are well known in the art. See, for example, International Patent Application No. WO2017037083.

An effective amount of a Cbl-b inhibitor is the amount or concentration of the Cbl-b inhibitor that is sufficient to modulate the activity of the immune cell as compared to a reference sample. The reference sample may be immune cells that have not been contacted with the Cbl-b inhibitor. In some embodiments, the concentration of a Cbl-b inhibitor added to a composition (e.g., cell culture medium) comprising the immune cells to be modified is from about 1 μM to about 100 μM, about 5 μM to about 100 μM, about 10 μM to about 100 μM, about 20 μM to about 100 μM, about 40 μM to about 100 μM, about 60 μM to about 100 μM, about 80 μM to about 100 μM, about 1 nM to about 100 μM, about 3 nM to about 100 μM, about 10 nM to about 100 μM, about 15 nM to about 100 μM, about 20 nM to about 100 μM, about 40 nM to about 100 μM, about 60 nM to about 100 μM, about 80 nM to about 100 μM, about 0.1 μM to about 100 μM, about 0.1 μM to about 90 μM, about 0.1 μM to about 80 μM, about 0.1 μM to about 70 μM, about 0.1 μM to about 60 μM, about 0.1 μM to about 50 μM, about 0.1 μM to about 40 μM, about 0.1 μM to about 30 μM, about 0.1 μM to about 20 μM, about 0.1 μM to about 10 μM, about 0.2 μM to about 10 μM, or about 0.3 μM to about 8 μM. In some embodiments, the concentration of a Cbl-b inhibitor added to a composition (e.g., cell culture medium) comprising the immune cells to be modified is about 1 μM, about 2 μM, about 3 μM, about 4 μM, about 5 μM, about 10 μM, about 20 μM, about 30 μM, about 40 μM, about 50 μM, about 60 μM, about 70 μM, about 80 μM, about 90 μM, about 1 nM, about 3 nM, about 5 nM, about 10 nM, about 20 nM, about 40 nM, about 50 nM, about 80 nM, about 0.1 μM, about 0.2 μM, about 0.3 μM, about 0.4 μM, about 0.5 μM, about 1 μM, about 5 μM, about 10 μM, about 15 μM, about 20 μM, about 25 μM, about 30 μM, about 40 μM, about 50 μM, about 60 μM, about 70 μM, about 80 μM, about 90 μM, or about 100 μM. In some embodiments, the concentration of a Cbl-b inhibitor added to a composition (e.g., cell culture medium) comprising the immune cells to be modified is about 0.3 μM, about 1 μM, or about 4 μM. In some embodiments, the concentration of a Cbl-b inhibitor added to a composition (e.g., cell culture medium) comprising the immune cells to be modified is about 1 μM or about 8 μM.

The effective amount of a Cbl-b inhibitor is in contact with the immune cells for a sufficient time to modulate the activity of the immune cell as compared to a reference sample. The reference sample may be immune cells that have not been contacted with the Cbl-b inhibitor but are incubated for the same length of time as the composition (e.g., cell culture medium) comprising the immune cells and the Cbl-b inhibitor. In some embodiments, the Cbl-b inhibitor is in contact and/or is incubated with the immune cells from about 1 minute to about 1 hour, about 5 minutes to about 1 hour, about 10 minutes to about 1 hour, about 15 minutes to about 1 hour, about 20 minutes to about 1 hour, about 30 minutes to about 1 hour, about 45 minutes to about 1 hour, about 1 hour to about 2 hours, about 1 hour to about 4 hours, about 1 hour to about 6 hours, about 1 hour to about 8 hours, about 1 hour to about 12 hours, about 1 hour to about 24 hours, about 2 hours to about 24 hours, about 6 hours to about 7 hours, about 6 hours to about 24 hours, about 8 hours to about 24 hours, about 10 hours to about 24 hours, about 15 hours to about 24 hours, about 20 hours to about 24 hours, about 12 hours to about 48 hours, about 24 hours to about 48 hours, or about 36 hours to about 48 hours. In some embodiments, the Cbl-b inhibitor is in contact and/or is incubated with the immune cells from about 1 day to about 7 days, about 2 days to about 7 days, about 3 days to about 7 days, about 4 days to about 7 days, about 5 days to about 7 days, or about 6 days to about 7 days. In some embodiments, the Cbl-b inhibitor is in contact and/or is incubated with the immune cells for about 1 minute, about 5 minutes, about 10 minutes, about 15 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, or about 24 hours. In some embodiments, the Cbl-b inhibitor is in contact and/or is incubated with the immune cells for about 1 day, about 2 days, about 3 days, about 4 days, about 5 days, about 6 days or about 7 days.

In some embodiments, the immune cells or a cell population comprising the immune cells are incubated under a suitable condition to induce proliferation, expansion, activation, and/or survival of the immune cells. Suitable conditions during incubation include, but are not limited to, use of one or more medium (e.g., cell culture medium), temperature, incubation time, the presence of a stimulating agent (e.g., anti-CD3 and/or anti-CD28 antibody), and present of any other beneficial agents or factors such as cytokines, chemokines, and/or recombinant soluble receptors.

In some embodiments, a suitable condition to induce proliferation, expansion, activation, and/or survival of the immune cells includes the provision of stimulating conditions or agents, which is capable of activating the immune cell (e.g., NK cell). For example, a suitable condition to induce proliferation, expansion, activation, and/or survival of a T-cell includes the provision of stimulating conditions or agents, which are capable of activating intracellular signaling in the T-cell. Full activation of T-cells generally requires the recognition of antigen by the T-cell receptor, referred to herein as “TCR” (signal one) as well as recognition of costimulators such as CD28 (signal two). In some aspects, one or more agent turns on or initiates a TCR complex mediated intracellular signaling cascade in a T-cell. For example, a first agent can bind to a component of the TCR complex in order to activate the T-cell and a second agent can bind to a costimulatory molecule on the surface of the T-cell to thereby stimulate the activated T-cell. In some embodiments, the first agent stimulated a TCR/CD3 complex-associated signal in the T-cell by specifically binding to CD3 (e.g., an anti-CD3 antibody). In a further embodiment, the co-stimulatory molecule on the surface of the T-cell may be CD28 and the second agent specifically binds to CD28 (e.g., anti-CD28 antibody). Such agents include, but are not limited to, antibodies, divalent antibody fragments, and binding molecules such as those specific for a TCR complex component (e.g., anti-CD3 antibody) and/or those specific for costimulatory receptor (e.g., anti-CD28 antibody). In some embodiments, an agent that specifically binds to CD3 is an anti-CD3 antibody, a divalent antibody fragment of an anti-CD3 antibody (e.g., (Fab)2′ fragment or a divalent scFv fragment), a monovalent antibody fragment of an anti-CD3 antibody (e.g., a Fab fragment, a Fv fragment, or a scFv fragment), or a CD3 binding molecule (e.g., an aptamer). In some embodiments, an agent that specifically binds to CD28 is an anti-CD28 antibody, a divalent antibody fragment of an anti-CD28 antibody (e.g., (Fab)2′ fragment or a divalent scFv fragment), a monovalent antibody fragment of an anti-CD28 antibody (e.g., a Fab fragment, a Fv fragment, or a scFv fragment), and a CD28 binding molecule (e.g., an aptamer). The one or more agents provided herein (e.g., anti-CD3 antibody and anti-CD28 antibody) for example, can be bound to a solid support such as a bead. In some embodiments, the expansion method step may further comprise the step of adding anti-CD3 antibody and/or anti-CD28 antibody to the culture medium. In some embodiments, the stimulating agents added to the cell culture medium include one or more cytokines such as, but not limited to, IL-2 and/or IL-15. For example, IL-2 can be added at a concentration of at least about 10 units/mL to a cell culture medium comprising the immune cells and agents such as anti-CD3 antibodies and/or anti-CD28 antibodies.

In some embodiments, a suitable condition to induce proliferation, expansion, activation, and/or survival of a T-cell includes the provision of stimulating conditions or agents which are capable of activating intracellular signaling through the T-cell receptor (TCR) complex, and a Cbl-b inhibitor as described herein. In some embodiments, the immune cells or a cell population comprising the immune cells are incubated with a first agent that stimulates a TCR/CD3 complex-associated signal in the T cell by specifically binding to CD3 (e.g., an anti-CD3 antibody). In a further embodiment, the immune cells or a cell population comprising the immune cells are incubated with a first agent that stimulates a TCR/CD3 complex-associated signal in the T cell by specifically binding to CD3 (e.g., an anti-CD3 antibody), with a second agent that binds to the co-stimulatory molecule CD28 (e.g., an anti-CD28 antibody), and with a Cbl-b inhibitor at a concentration of about 1 μM to about 100 μM (e.g., about 0.3 μM, about 1 μM, or about 4 μM). In some embodiments a suitable condition to induce proliferation, expansion, activation, and/or survival of a T-cell when in the presence of a Cbl-b inhibitor does not require stimulation through a co-stimulatory molecule (e.g., CD28). Contacting T-cells with a Cbl-b inhibitor or a composition thereof can bypass the need for co-stimulation required for T-cells to enter into an activated state. In some such embodiments, the immune cells or a cell population comprising the immune cells are incubated with a first agent that stimulates a TCR/CD3 complex-associated signal in the T-cell by specifically binding to CD3 (e.g., an anti-CD3 antibody) and with a Cbl-b inhibitor at a concentration of about 0.1 μM to about 50 μM (e.g., about 1 μM or about 8 μM).

In some embodiments of the methods for modulating activity of an immune cell, the immune cell is a T-cell and modulating activity of the T-cell comprises increased T-cell activation and/or increased T-cell proliferation. T-cells contemplated in embodiments herein may be in a tolerant state even in the presence of an activating agent that binds to a component of the TCR complex, such as an anti-CD3 antibody, as well as in the presence of a stimulating agent that binds a co-stimulatory molecule, such as an anti-CD28 antibody. In some embodiments, the method of modulating activity of a T-cell comprises contacting the T-cell with an effective amount of a Cbl-b inhibitor or a composition thereof in the presence of an anti-CD3 antibody in combination with an anti-CD28 antibody. In some embodiments, the method of modulating activity of a T-cell comprises contacting the T-cell with an effective amount of a Cbl-b inhibitor or a composition thereof, wherein the T-cell previously has been in contact with an anti-CD3 antibody in combination with an anti-CD28 antibody. In some embodiments, stimulation via the co-stimulatory CD28 molecule is not required for modulating the activity of the T-cell (e.g., increasing T-cell activation and/or increasing T-cell proliferation). In some embodiments, the method of modulating activity of a T-cell comprises contacting the T-cell with an effective amount of a Cbl-b inhibitor or a composition thereof in the presence of an anti-CD3 antibody alone. In some embodiments, the method of modulating activity of a T-cell comprises contacting the T-cell with an effective amount of a Cbl-b inhibitor or a composition thereof, wherein the T-cell has previously been in contact with one or more agents that activate the T-cell (e.g., an anti-CD3 antibody), wherein said agents do not include an agent that stimulates the CD28 co-stimulatory molecule (e.g., an anti-CD28 antibody).

In some embodiments, the immune cell is a T-cell and modulating activity of the T-cell comprises enhanced T-cell activation and/or enhanced T-cell proliferation. For example, T-cells contemplated in embodiments herein may be in an activated state such as when in the presence of agents that activate the T-cells (e.g., anti-CD3 antibody), and in some further embodiments, in the presence of agents that stimulate the T-cells (e.g., anti-CD28 antibody). Contacting T-cells with a Cbl-b inhibitor or composition thereof can lower the threshold required for activation and therefore enhance activation and/or proliferation of T-cells that are in the presence of an activating agent (e.g., an anti-CD3 antibody) and in some further embodiments, a stimulating agent (e.g., an anti-CD28 antibody). In some embodiments, the method of modulating activity of a T-cell comprises contacting the T-cell with an effective amount of a Cbl-b inhibitor or a composition thereof in the presence of an anti-CD3 antibody in combination with an anti-CD28 antibody. In some embodiments, the method of modulating activity of a T-cell comprises contacting the T-cell with an effective amount of a Cbl-b inhibitor or a composition thereof, wherein the T-cell has previously been in contact with an anti-CD3 antibody in combination with an anti-CD28 antibody. In some embodiments, stimulation via the co-stimulatory CD28 molecule is not required for modulating the activity of the T-cell (e.g., enhancing T-cell activation and/or enhancing T-cell proliferation). In some embodiments, the method of modulating activity of a T-cell comprises contacting the T-cell with an effective amount of a Cbl-b inhibitor or a composition thereof in the presence of an anti-CD3 antibody alone. In some embodiments, the method of modulating activity of a T-cell comprises contacting the T-cell with an effective amount of a Cbl-b inhibitor or a composition thereof, wherein the T-cell has previously been in contact with one or more agents that activate the T-cell (e.g., anti-CD3 antibody).

In some embodiments, the immune cell is a T-cell and modulating activity of the T-cell comprises decreased T-cell dysfunction including decreased T-cell exhaustion, decreased T-cell tolerance, and/or decreased T-cell anergy. General principles of T-cell dysfunction are well known in the art. See Schietinger, A. et al., Trends Immunol., 35(2):51-60 (2014). Immune tolerance is a process that is part of the normal function of the immune system. Antigen-specific immune tolerance is characterized by a decrease in responsiveness to an antigen, which is induced by previous exposure to that antigen. When specific lymphocytes (e.g., T-cells) encounter antigens, the lymphocytes may be activated, leading to an antigen-specific immune response, or the lymphocytes (e.g., T-cells) may be inactivated or eliminated, leading instead to antigen-specific immune tolerance. In some aspects, tolerance can be caused by clonal anergy, peripheral clonal deletion, suppression of T-cells and/or other forms of antigen-specific tolerance. In some embodiments, tolerance may result from or be characterized by the induction of anergy. In some aspects, anergy can result by exposure of T-cells to an antigen in the absence of costimulation. Prolonged antigen recognition by the TCR alone, in the absence of the co-stimulatory signal, may lead to anergy (i.e., functional unresponsiveness). Anergic T cells may be refractory to subsequent antigenic challenge, and may be capable of suppressing other immune responses. Generally, in the natural setting tolerance is involved in non-reactivity or nonproductive reactivity to self-antigens. In some cases, however, tolerance to a “non-self” antigen can be induced. Thus, in some aspects, the same mechanisms by which mature T-cells that recognize self-antigens in peripheral tissues become incapable of subsequently responding to these antigens also may regulate unresponsiveness to foreign or “non-self” antigens such as those expressed by cancer cells. Accordingly, T-cells contemplated in embodiments herein may be in a tolerant state even in the presence of stimulatory agents such as agents that binds to a co-stimulatory molecule such as CD28. Contacting T-cells with a Cbl-b inhibitor provided herein or a composition thereof can bypass aspects of T-cell dysfunction such as T-cell tolerance, T-cell anergy and/or T-cell exhaustion. In some embodiments, the method of modulating activity of a T-cell (e.g., decreasing T-cell tolerance, decreasing T-cell anergy and/or decreasing T-cell exhaustion) comprises contacting the T-cell with an effective amount of a Cbl-b inhibitor or a composition thereof. In some embodiments of the methods herein, modulating activity of a T-cell (e.g., decreasing T-cell tolerance, decreasing T-cell anergy and/or decreasing T-cell exhaustion) comprises contacting the T-cell with an effective amount of a Cbl-b inhibitor or a composition thereof in the presence of an anti-CD3 antibody in combination with an anti-CD28 antibody. In some embodiments of the methods herein, the method of modulating activity of a T-cell (e.g., decreasing T-cell tolerance, decreasing T-cell anergy and/or decreasing T-cell exhaustion) comprises contacting the T-cell with an effective amount of a Cbl-b inhibitor or a composition thereof, wherein the T-cell previously has been in contact with an anti-CD3 antibody in combination with an anti-CD28 antibody. In some embodiments, stimulation via the co-stimulatory CD28 molecule is not required for modulating the activity of the T-cell (e.g., decreasing T-cell tolerance, decreasing T-cell anergy and/or decreasing T-cell exhaustion). In some embodiments of the methods herein, the method of modulating activity of a T-cell (e.g., decreasing T-cell tolerance, decreasing T-cell anergy and/or decreasing T-cell exhaustion) comprises contacting the T-cell with an effective amount of a Cbl-b inhibitor or a composition thereof in the presence of an anti-CD3 antibody alone. In some embodiments, the method of modulating activity of a T-cell (e.g., decreasing T-cell tolerance, decreasing T-cell anergy and/or decreasing T-cell exhaustion) comprises contacting the T-cell with an effective amount of a Cbl-b inhibitor or a composition thereof, wherein the T-cell has previously been in contact with one or more agents that activate the T-cell, such as an anti-CD3 antibody alone.

T-cell activation and T-cell tolerance are tightly controlled processes regulating the immune response. Accordingly, provided herein are methods of modulating activity of the T-cell, wherein modulating activity of the T-cell comprises increased T-cell activation, increased T-cell proliferation, decreased T-cell exhaustion, and/or decreased T-cell tolerance. In some embodiments, the method of modulating activity of a T-cell (e.g., increased T-cell activation, increased T-cell proliferation, decreased T-cell exhaustion, and/or decreased T-cell tolerance) comprises contacting the T-cell with an effective amount of a Cbl-b inhibitor or a composition thereof. In some embodiments of the methods herein, modulating activity of a T-cell (e.g., increasing T-cell activation, increasing T-cell proliferation, decreasing T-cell exhaustion, and/or decreasing T-cell tolerance) comprises contacting the T-cell with an effective amount of a Cbl-b inhibitor or a composition thereof in the presence of an anti-CD3 antibody in combination with an anti-CD28 antibody. In some embodiments of the methods herein, the method of modulating activity of a T-cell (e.g., increasing T-cell activation, increasing T-cell proliferation, decreasing T-cell exhaustion, and/or decreasing T-cell tolerance) comprises contacting the T-cell with an effective amount of a Cbl-b inhibitor or a composition thereof, wherein the T-cell previously has been in contact with an anti-CD3 antibody in combination with an anti-CD28 antibody. In some embodiments, stimulation via the co-stimulatory CD28 molecule is not required for modulating the activity of the T-cell (e.g., increasing T-cell activation, increasing T-cell proliferation, decreasing T-cell exhaustion, and/or decreasing T-cell tolerance). In some embodiments of the methods herein, the method of modulating activity of a T-cell (e.g., increasing T-cell activation, increasing T-cell proliferation, decreasing T-cell exhaustion, and/or decreasing T-cell tolerance) comprises contacting the T-cell with an effective amount of a Cbl-b inhibitor provided herein or a composition thereof in the presence of an anti-CD3 antibody alone. In some embodiments, the method of modulating activity of a T-cell (e.g., increasing T-cell activation, increasing T-cell proliferation, decreasing T-cell exhaustion, and/or decreasing T-cell tolerance) comprises contacting the T-cell with an effective amount of a Cbl-b inhibitor or a composition thereof, wherein the T-cell has previously been in contact with one or more agents that activate the T-cell (e.g., an anti-CD3 antibody).

In some embodiments of the methods herein, increased T-cell activation comprises increased production of one or more cytokines from T-cells or surrounding immune cells in the activated T-cell microenvironment (e.g., myeloid cells). In some embodiments, the one or more cytokines include, but are not limited to: IFN-γ, IL-1β, IL-2, IL-4, IL-5, IL-6, IL-13, IL-18, TNFα, and GM-CSF. In some embodiments, the cytokine is one or more of: IL-2, IFN-γ, TNFα, and GM-CSF. In some embodiments, the cytokine is a chemokine. In some embodiments, the one or more chemokines include, but are not limited to: IP-10, Eotaxin, GRO alpha, RANTES, MIP-1α, MIP-1β, MIP-2, MCP-1, and MCP-3. Increased expression of cytokines can be measured by ELISA.

In some embodiments of the methods herein, increased T-cell activation comprises increased cell surface expression of one or more T-cell activation markers. In some embodiments, the one or more T-cell activation markers include, but are not limited to: CD25, CD44, CD62L, CD69, CD152 (CTLA4), CD154, CD137, and CD279. In some embodiments, the T-cell activation marker is one or more of: CD25, CD69, and CTLA4. Increased expression of cell surface markers can be measured by FACS.

Methods for experimentally determining increased T-cell activation, increased T-cell proliferation, decreased T-cell exhaustion, and/or decreased T-cell tolerance are well known in the art. In some embodiments, representative methods of determining T-cell activation can be found in Biological Example 2 provided herein. In some embodiments, representative in vitro and in vivo methods of determining increased T-cell activation, increased T-cell proliferation, decreased T-cell exhaustion, and/or decreased T-cell tolerance can be found in Biological Example 3 provided herein.

In some embodiments of the methods for modulating activity of an immune cell, the immune cell is a B cell and modulating activity of the B cell comprises increased B cell activation. In some embodiments, increased B cell activation comprises increased cell surface expression of one or more B cell activation markers. In some embodiments, the one or more B cell activation markers include, but are not limited to: CD69, CD86, and MHC class II (e.g., HLA-DR). In some embodiments, the B cell activation marker is CD69. Increased expression of cell surface markers can be measured by FACS. In some embodiments, increased B cell activation comprises increased activation of proteins in signaling pathways such as those mediated by ERK, JNK, and Syk. Increased activation of said proteins can be detected by measurement of levels of phosphorylation on the proteins using reagents such as anti-phospho antibodies available in the art.

In some embodiments of the methods for modulating activity of an immune cell, the immune cell is a NK cell and modulating activity of the NK cell comprises increased NK cell activation. In some embodiments, increased NK cell activation comprises secretion of one or more cytokines. In some embodiments, the one or more cytokines include, but are not limited to: IFN-γ, TNFα, and MIP-1β. Increased expression of cytokines can be measured by ELISA. In some embodiments, increased NK cell activation comprises increased cell surface expression of one or more NK cell activation markers. In some embodiments, the one or more NK cell activation markers include, but are not limited to: CD69, and CD107a. Increased expression of cell surface markers can be measured by FACS. In some embodiments, increased NK cell activation comprises increased killing of target cells such as tumor cells, including primary tumor cells, and cell line derived tumor cells such as the K562 cell line.

Methods for experimentally determining increased B cell activation and NK cell activation are well known in the art. See, for example, Fauriat et al., Blood. 115(11):2167-76, (2010); Beano et al., J. Transl. Med. 6:25, (2008); Claus et al., J. Immunol. Methods, 341(1-2): 154-64, (2009); and Fujisaki et al., Cancer Res. 69(9): 4010-4017, (2009). In some embodiments, representative methods of determining B-cell activation can be found in Biological Example 3 provided herein. In some embodiments, representative methods of determining NK cell activation can be found in Biological Example 3 provided herein.

Modulation of activity of an immune cell, such as a T-cell, a B cell or a NK cell can be measured by determining a baseline value for a parameter of interest (e.g., cytokine secretion). For example, T-cell activation, such as in a sample obtained from in vitro experiments of cells contacted with a Cbl-b inhibitor, can be measured before contacting or administering said Cbl-b inhibitor to determine a baseline value. A reference value then is obtained for T-cell activation after contacting or administering said Cbl-b inhibitor. The reference value is compared to the baseline value in order to determine the amount of T-cell activation due to contact or administration of the Cbl-b inhibitor or composition thereof. For example, in some embodiments, immune cell (e.g., T-cell) activation is increased by at least 0.1-fold in a sample as compared to a baseline value, wherein the baseline value is obtained before contacting the immune cell (e.g., T-cell) with a Cbl-b inhibitor or a composition thereof. In some embodiments, immune cell (e.g., T-cell) activation is increased by at least about 0.1-fold, about 0.2-fold, about 0.3-fold, about 0.4-fold, about 0.5-fold, about 0.6-fold, about 0.7-fold, about 0.8-fold, about 0.9-fold, about 1-fold, about 2-fold, about 4-fold, about 6-fold, about 8-fold, about 10-fold, about 20-fold, about 30-fold, but no more than about 50-fold over a baseline value. Immune cell activation can be assessed by measuring biological markers of activation such as increased cytokine secretion, increased cell surface expression of activation markers (e.g., cell surface markers), or increased phosphorylation of proteins in a downstream signaling pathway. The fold over baseline value that indicates immune cell activation can be determined for the parameter being tested and the conditions under which the immune cell were treated. For example, for measuring T-cell activation, a baseline value can be obtained from T-cells stimulated with anti-CD3 antibody in combination with anti-CD28 antibody, wherein the cells are not incubated with a Cbl-b inhibitor. A reference value is then obtained from T-cells stimulated with anti-CD3 antibody in combination with anti-CD28 antibody, wherein the T-cells have been or are in contact with a Cbl-b inhibitor. A positive response for immune cell activation can then be determined by the obtained reference value. Similar reference value measurements can be obtained and compared to a baseline value for assessing T-cell activation, T-cell proliferation, T-cell exhaustion, T-cell tolerance, B cell activation and/or NK cell activation. Measurements for these parameters can be obtained utilizing techniques well known in the art, as well as the techniques provided in Biological Examples 2 and 3.

The terms “baseline” or “baseline value” as used herein can refer to a measurement or characterization before administration of a therapeutic agent as disclosed herein (e.g., a composition comprising a Cbl-b inhibitor as described herein) or at the beginning of administration of the therapeutic agent. The baseline value can be compared to a reference value in order to determine the increase or decrease of an immune cell function (e.g., increasing T-cell activation, increasing T-cell proliferation, decreasing T-cell exhaustion, and/or decreasing T-cell tolerance). The terms “reference” or “reference value” as used herein can refer to a measurement or characterization after administration of the therapeutic agent as disclosed herein (e.g., a composition comprising a Cbl-b inhibitor as described herein). The reference value can be measured one or more times during an experimental time course, dosage regimen, or treatment cycle, or at the completion of the experimental time course, dosage regimen or treatment cycle. A “reference value” can be an absolute value, a relative value, a value that has an upper and/or lower limit, a range of values, an average value, a median value, a mean value, or a value as compared to a baseline value. Similarly, a “baseline value” can be an absolute value, a relative value, a value that has an upper and/or lower limit, a range of values, an average value, a median value, a mean value, or a value as compared to a reference value. The reference value and/or baseline value can be obtained from one sample (e.g., one sample obtained from an individual), from two different samples (e.g., a sample obtained from two different individuals) or from a group of samples (e.g., samples obtained from a group of two, three, four, five or more individuals).

In some embodiments, a positive response for T-cell activation as measured by cytokine secretion (e.g., IL-2 secretion) by T-cells stimulated with anti-CD3 antibody in combination with anti-CD28 antibody in the presence of a Cbl-b inhibitor is at least 2.5-fold over the baseline value for cytokine secretion (e.g., IL-2 secretion) obtained from T-cells stimulated with anti-CD3 antibody in combination with anti-CD28 antibody in the absence of a Cbl-b inhibitor. In some embodiments, a positive response for T-cell activation as measured by surface marker expression (e.g., CD25 surface marker staining) by T-cells stimulated with anti-CD3 antibody in combination with anti-CD28 antibody in the presence of a Cbl-b inhibitor is at least 1.3-fold over the baseline value for surface marker expression (e.g., CD25 surface marker staining) obtained from T-cells stimulated with anti-CD3 antibody in combination with anti-CD28 antibody in the absence of a Cbl-b inhibitor. In some embodiments, a baseline value can be obtained from T-cells stimulated with anti-CD3 antibody alone, wherein the cells are not incubated with a Cbl-b inhibitor. In some embodiments, a positive response for T-cell activation as measured by cytokine secretion (e.g., IL-2 secretion) by T-cells stimulated with anti-CD3 antibody alone in the presence of a Cbl-b inhibitor is at least 0.1-fold over the baseline value for cytokine secretion (e.g., IL-2 secretion) obtained from T-cells stimulated with anti-CD3 antibody alone in the absence of a Cbl-b inhibitor. In some embodiments, a positive response for T-cell activation as measured by surface marker expression (e.g., CD25 surface marker staining) by T-cells stimulated with anti-CD3 antibody alone in the presence of a Cbl-b inhibitor is at least 0.6-fold over the baseline value for surface marker expression (e.g., CD25 surface marker staining) obtained from T-cells stimulated with anti-CD3 antibody alone in the absence of a Cbl-b inhibitor.

In some aspects, provided herein are methods of producing a modified immune cell, comprising culturing a cell population containing an immune cell in the presence of an effective amount of a Cbl-b inhibitor provided herein or a composition thereof to modulate the activity of the immune cell, thereby producing the modified immune cell. In some embodiments, the immune cell is a T-cell, a B cell, or a natural killer (NK) cell.

In some embodiments of the methods for producing a modified immune cell, the immune cell that is to be modified is a cell selected from the group consisting of: a hematopoietic cell, a multipotent stem cell, a myeloid progenitor cell, a lymphoid progenitor cell, a T-cell, a B cell, and a NK cell. In some embodiments, the method further comprises culturing the immune cell with stimulating agents such as cytokines or antibodies that bind to activating proteins expressed by the immune cell (e.g., an anti-CD3 antibody and/or an anti-CD28 antibody). In some embodiments, the immune cell that is to be modified is in a cell population containing the immune cell, wherein the cell population is obtained as a sample from an individual. In some embodiments, the immune cell that is to be modified is in a cell population containing the immune cell, wherein the cell population is obtained from culturing a biological sample (e.g., blood sample, bone marrow sample, etc.) from an individual. In some embodiments, the immune cell is modified by contacting the cell population containing the immune cell with a Cbl-b inhibitor or composition thereof thereby producing a modified immune cell. In some embodiments, the modified immune cell is a cell selected from the group consisting of: a hematopoietic cell, a multipotent stem cell, a myeloid progenitor cell, a lymphoid progenitor cell, a T-cell, a B cell, and a NK cell. In some embodiments, the immune cell is the same cell type as the modified immune cell. For example, the immune cell can be an inactive T-cell and the modified immune cell can be an activated T-cell. In some embodiments, the immune cell is a different cell type than the modified immune cell. For example, the immune cell can be a hematopoietic stem cell and the modified immune cell can be an NK cell that has differentiated from the hematopoietic stem cell. In some embodiments of the method of producing the modified immune cell, the method further comprises recovering the modified immune cell. In some embodiments, the cell population containing the immune cell, the immune cell or the modified immune cell is from an individual (e.g., a human). In some embodiments, the immune cell or modified immune cell is a human immune cell or human modified immune cell, respectively.

Further provided herein are modified immune cells produced by any of the methods described herein such as culturing a cell population containing an immune cell in the presence of an effective amount of a Cbl-b inhibitor to modulate the activity of the immune cell and thereby produce the modified immune cell.

In some embodiments, the Cbl-b inhibitors provided herein are cell membrane permeable. Accordingly, in some embodiments, a modified immune cell provided herein can comprise a Cbl-b inhibitor described herein such as in the cytoplasm of the modified immune cell.

In some aspects, provided herein is an isolated modified immune cell, wherein the modified immune cell has been contacted or is in contact with a Cbl-b inhibitor described herein or a composition thereof. In some embodiments, the modified immune cell is a T-cell, a B cell, or a natural killer (NK) cell. In some embodiments, the modified immune cell is a hematopoietic cell, a multipotent stem cell, a myeloid progenitor cell, a lymphoid progenitor cell, a T-cell, a B cell, or a NK cell.

In some embodiments of the isolated modified immune cell, the modified immune cell is a T-cell, and the T-cell exhibits increased T-cell activation, increased T-cell proliferation, decreased T-cell exhaustion, and/or decreased T-cell tolerance. In some embodiments, increased T-cell activation comprises increased production of one or more cytokines from T-cells or surrounding immune cells in the activated T-cell microenvironment (e.g., myeloid cells). In some embodiments, the one or more cytokines include, but are not limited to: IFN-γ, IL-1β, IL-2, IL-4, IL-5, IL-6, IL-13, IL-18, TNFα, and GM-CSF. In some embodiments, the one or more cytokines is one or more selected from the group consisting of: IL-2, IFN-γ, TNFα, and GM-CSF. In some embodiments, the cytokine is a chemokine. In some embodiments, the one or more chemokines include, but are not limited to: IP-10, Eotaxin, GRO alpha, RANTES, MIP-la, MIP-1β, MIP-2, MCP-1, and MCP-3. In some embodiments, increased T-cell activation comprises increased cell surface expression of one or more T-cell activation markers. In some embodiments, the one or more T-cell activation markers include, but are not limited to: CD25, CD44, CD62L, CD69, CD152 (CTLA4), CD154, CD137, and CD279. In some embodiments, the one or more T-cell activation markers include, but are not limited to: CD25, CD69, and CTLA4. In some embodiments, the T-cell activation markers are CD25 and/or CD69. In some embodiments, the T-cell has been or is in contact with an anti-CD3 antibody. In some embodiments, the T-cell has been or is in contact with an anti-CD3 antibody in combination with an anti-CD28 antibody.

In some embodiments of the isolated modified immune cell, the modified immune cell is a NK cell, and the NK cell exhibits increased NK cell activation. In some embodiments, increased NK cell activation comprises increased secretion of one or more cytokines (e.g., IFN-γ, TNFα, and/or MIP-1β). In some embodiments, increased NK cell activation comprises increased cell surface expression of one or more NK cell activation markers (e.g., CD69 and/or CD107a).

In some embodiments of the isolated modified immune cell, the modified immune cell is a B cell, and the B cell exhibits increased B cell activation. In some embodiments, increased B cell activation comprises increased cell surface expression of one or more B cell activation markers (e.g., CD69, CD86, and/or HLA-DR).

In some of any embodiments of the methods or modified immune cells provided herein, the immune cell or modified immune cell is a mammalian cell (e.g., human cell). In some embodiments, the immune cell or modified immune cell is a human cell.

In some aspects, incubation is carried out in accordance with techniques such as those described in U.S. Pat. No. 6,040,177; Klebanoff et al., J Immunother., 35(9):651-660 (2012), Terakura et al., Blood. 1:72-82, (2012), or Wang et al., J Immunother., 35(9):689-701 (2012).

The immune cells to be modified or modified immune cells provided herein can be engineered to express a recombinant chimeric receptor such as a chimeric antigen receptor (CAR). In some embodiments, the CAR comprises from its N terminus to C terminus: an extracellular ligand-binding domain, a transmembrane domain, an intracellular co stimulatory domain and an activating cytoplasmic signaling domain. In some embodiments, the CAR comprises from its N terminus to C terminus: an extracellular ligand-binding domain, a transmembrane domain and an activating cytoplasmic signaling domain. The immune cells can be engineered to express the recombinant chimeric receptor (e.g., CAR) before, during, or after contact with a Cbl-b inhibitor provided herein. In some embodiments, an immune cell to be modified is a T-cell (e.g., a CD4⁺ T-cell or a CD8⁺ T-cell). In a further embodiment, the T-cell comprises a recombinant chimeric receptor such as a CAR. In some embodiments, the modified immune cell is a modified T-cell (e.g., a CD4⁺ T-cell or a CD8⁺ T-cell). In a further embodiment, the modified T-cell comprises a recombinant chimeric receptor such as a CAR. Methods for producing immune cells expressing recombinant chimeric receptors are well known in the art such as by the introduction of a nucleic acid encoding the recombinant chimeric receptor (e.g., CAR) to an immune cell (e.g., T-cell) via a vector (e.g., viral vector). See, for example, see International Patent Application No. WO2017096329 and U.S. patent Application No. US20170204372.

In some embodiments, methods for isolation and processing of immune cells to be modified or which have been modified (i.e., modified immune cells) include steps for freezing (e.g., cryopreserving) the cells, either before or after isolation, incubation (e.g., incubation with a Cbl-b inhibitor), and/or engineering (e.g., introduction of a nucleic acid encoding a recombinant chimeric receptor to the immune cell). A variety of freezing solutions and parameters known in the art may be used.

B. Adoptive Cell Therapy

The modified immune cells or compositions thereof produced by the methods herein can be used as a therapeutic agent in methods of treatment of an individual in need thereof such as an individual having cancer. Such methods of treatment include adoptive cell therapy. In some embodiments, the method of treatment includes isolating cells from an individual, preparing, processing, culturing, and/or engineering them, as described herein, and re-introducing them into the same individual, before or after cryopreservation. In some embodiments, the method of treatment include isolating cells from an individual, preparing, processing, culturing, and/or engineering them, as described herein, and re-introducing them into a different individual, before or after cryopreservation.

Accordingly, in some aspects, provided herein is a method of modulating the immune response in an individual, the method comprising administering an effective amount of a modified immune cell described herein or a composition thereof to an individual in need thereof (e.g., an individual with a T-cell dysfunction disorder). In some embodiments, the individual has a cancer. In some embodiments, provided herein is a method of treating a cancer responsive to inhibition of Cbl-b activity, the method comprising administering an effective amount of a modified immune cell described herein or a composition thereof to an individual having the cancer responsive to inhibition of Cbl-b activity. In some embodiments, provide herein is a method of inhibiting abnormal cell proliferation, the method comprising administering an effective amount of a modified immune cell described herein or a composition thereof to an individual in need thereof.

The term “abnormal cell proliferation” as used herein includes hyperplasia or cancer cell proliferation. The cancer cell may be derived from a hematologic cancer or a non-hematologic cancer such as those described herein.

In some embodiments of the methods herein, the cancer is a hematologic cancer such as lymphoma, a leukemia or a myeloma. A hematologic cancer contemplated herein includes, but is not limited to, one or more leukemias such as B-cell acute lymphoid leukemia (“BALL”), T-cell acute lymphoid leukemia (“TALL”), acute lymphoid leukemia (ALL); one or more chronic leukemias including but not limited to chronic myelogenous leukemia (CML) and chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia,” which are a diverse collection of hematological conditions united by ineffective production (or dysplasia) of myeloid blood cells.

In some embodiments of the methods herein, the cancer is a non-hematologic cancer such as a sarcoma, a carcinoma, or a melanoma. A non-hematologic cancer contemplated herein includes, but is not limited to, a neuroblastoma, renal cell carcinoma, colon cancer, colorectal cancer, breast cancer, epithelial squamous cell cancer, melanoma, stomach cancer, brain cancer, lung cancer (e.g., NSCLC), pancreatic cancer, cervical cancer, ovarian cancer, liver cancer, bladder cancer, prostate cancer, testicular cancer, thyroid cancer, uterine cancer, adrenal cancer and head and neck cancer.

In certain embodiments, an individual in need thereof of treatment, such as an individual having cancer or a T-cell dysfunction disorder, is administered a composition comprising the modified immune cells provided herein at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

The modified immune cells and compositions thereof are administered using standard administration techniques, formulations, and/or devices. Provided are formulations and devices, such as syringes and vials, for storage and administration of the compositions. Formulations or pharmaceutical composition comprising the modified immune cells include those for intravenous, intraperitoneal, subcutaneous, or intramuscular administration. In some embodiments, the modified immune cells are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injections. Compositions of the modified immune cells can be provided as sterile liquid preparations, e.g., isotonic aqueous solutions, suspensions, emulsions, dispersions, or viscous compositions, which may in some aspects be buffered to a selected pH. Viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the modified immune cells in a solvent, such as in admixture with a suitable carrier, diluent, or excipient such as sterile water, physiological saline, glucose, dextrose, or the like.

In some embodiments, the modified immune cells are co-administered with one or more additional therapeutic agents or in connection with another therapeutic intervention, either simultaneously or sequentially in any order. For instance, in some therapeutic regimens of the present disclosure, both the modified immune cells and a Cbl-b inhibitor are administered to a mammalian subject in need thereof, wherein the Cbl-b inhibitor is a compound of Formula (I-A), Formula (I), Formula (II-A), Formula (II), Formula (III-A), Formula (III), Formula (VI), or Formula (IV-ox) of any one of the exemplary embodiments or claims. Thus, in some embodiments the therapeutic regimens comprise both adoptive cell therapy and chemotherapy.

After the modified immune cells are administered to an individual (e.g., a human), the biological activity of the modified immune cell populations can be measured by methods known in the art. Parameters to assess include specific binding of modified immune cell or other immune cell to antigen, in vivo (e.g., by imaging) or ex vivo (e.g., by ELISA or flow cytometry). In some embodiments, the ability of modified immune cells to destroy target cells can be measured using any suitable method known in the art, such as cytotoxicity assays described in, for example, Kochenderfer et al., J. Immunotherapy, 32 (7): 689-702 (2009), and Herman et al. J. Immunological Methods, 285 (1): 25-40 (2004). In some embodiments, the biological activity of the modified immune cells also can be measured by assaying expression and/or secretion of certain cytokines, such as IL-2 and IFNγ. In some aspects the biological activity of the modified immune cells is measured by assessing clinical outcome, such as reduction in tumor size or number of tumors.

C. Administration of Cbl-b Inhibitor

In some aspects, a Cbl-b inhibitor or composition thereof can be administered directly to an individual to modulate an immune response, treat a disease or condition (e.g., cancer and/or abnormal cell proliferation) and/or inhibit Cbl-b activity in the individual.

In some embodiments, provided herein is a method of modulating the immune response, the method comprising administering an effective amount of a Cbl-b inhibitor provided herein or a composition thereof to an individual to modulate the immune response in the individual. In some embodiments, the individual has a cancer such as a hematologic cancer or non-hematological cancer described herein.

In some embodiments, provided herein is a method of treating cancer responsive to inhibition of Cbl-b activity, the method comprising administering an effective amount of a Cbl-b inhibitor provided herein or a composition thereof to an individual to treat the cancer responsive to inhibition of Cbl-b activity. In some embodiments, the cancer is a hematologic cancer or non-hematological cancer such as one described herein.

In some embodiments, provided herein is a method of inhibiting abnormal cell proliferation (e.g., hyperplasia), the method comprising administering an effective amount of a Cbl-b inhibitor provided herein or a composition thereof to an individual to inhibit abnormal cell proliferation in the individual.

In some embodiments, provided herein is a method of inhibiting Cbl-b activity, the method comprising administering an effective amount of a Cbl-b inhibitor provided herein or a composition thereof to an individual to inhibit Cbl-b activity in the individual.

In some embodiments, such as in the modulation of an immune response in an individual in need thereof (e.g., an individual with a T-cell dysfunction disorder), treatment of a disease or condition in an individual (e.g., an individual cancer and/or abnormal cell proliferation) and/or inhibition of Cbl-b activity in an individual, the appropriate dosage of an active agent, will depend on the type of condition, disease or disorder to be treated, as defined above, the severity and course of the condition, disease or disorder, whether the agent is administered for preventive or therapeutic purposes, previous therapy, the subject's clinical history and response to the Cbl-b inhibitor, and the discretion of the attending physician. The Cbl-b inhibitor or composition thereof is suitably administered to the individual at one time or over a series of treatments. In some embodiments, the treatment includes multiple administrations of the Cbl-b inhibitor or composition thereof, wherein the interval between administrations may vary. For example, the interval between the first administration and the second administration is about one month, and the intervals between the subsequent administrations are about three months. In some embodiments, a Cbl-b inhibitor described herein is administered at a flat dose. In some embodiments, a Cbl-b inhibitor described herein is administered to an individual at a fixed dose based in the individual's weight (e.g., mg/kg).

In some embodiments, the Cbl-b inhibitor is co-administered with one or more additional therapeutic agents or in connection with another therapeutic intervention, either simultaneously or sequentially in any order. For instance, in some therapeutic regimens of the present disclosure, both the Cbl-b inhibitor and modified immune cells are administered to a mammalian subject in need thereof, wherein the Cbl-b inhibitor is a compound of Formula (I-A), Formula (I), Formula (II-A), Formula (II), Formula (III-A), Formula (III), Formula (VI) or Formula (IV-ox) of any one of the exemplary embodiments or claims. Thus, in some embodiments the therapeutic regimens comprise both adoptive cell therapy and chemotherapy.

In some aspects, the effectiveness of Cbl-b inhibitor administration in the treatment of a disease or disorder such as cancer is measured by assessing clinical outcome, such as reduction in tumor size or number of tumors, and/or survival.

In some embodiments, the effectiveness of Cbl-b inhibitor administration in the methods herein (e.g., method of modulating an immune response in an individual) can be assessed by measuring the biological activity immune cells present in a sample (e.g., blood sample) isolated from the treated individual. For example, the ability of immune cells isolated from the individual after treatment with a Cbl-b inhibitor to destroy target cells using a cytotoxicity assay. In some embodiments, the biological activity of immune cells present in a sample (e.g., blood sample) can be measured by assaying expression and/or secretion of certain cytokines, such as IL-2 and IFNγ.

### IV. Compositions, Formulations and Routes of Administration

Pharmaceutical compositions of any of the compounds disclosed herein, or a salt or solvate thereof, are embraced by this disclosure. Thus, the disclosure includes pharmaceutical compositions comprising a compound of Formula (I-A), Formula (I), Formula (II-A), Formula (II), Formula (III-A), Formula (III), Formula (IV), or Formula (IV-ox), or any variation thereof disclosed herein, or a pharmaceutically acceptable salt or solvate thereof, or tautomers thereof, or stereoisomers or mixtures of stereoisomers thereof, and a pharmaceutically acceptable excipient, such as a pharmaceutically acceptable vehicle or pharmaceutically acceptable carrier. In some embodiments, the compound is a compound selected from Compound Nos. 1-485 in Table 1 (including “a” and “b” variants listed in Table 1), or a pharmaceutically acceptable salt or solvate thereof, or tautomers thereof, or stereoisomers or mixtures of stereoisomers thereof. In one aspect, the pharmaceutically acceptable salt is an acid addition salt, such as a salt formed with an inorganic or organic acid.

The compounds and compositions disclosed herein may be administered in any suitable form and by any suitable route that will provide sufficient levels of the compounds for treatment of the disease or disorder. Such administration includes oral administration, enteral administration, parenteral administration including subcutaneous injection, intravenous injection, intraarterial injection, intramuscular injection, intrasternal injection, intraperitoneal injection, intralesional injection, intraarticular injection, intratumoral injection, or infusion techniques. The compounds and compositions also can be administered sublingually, by mucosal administration, by buccal administration, subcutaneously, by spinal administration, by epidural administration, by administration to cerebral ventricles, by inhalation (e.g., as mists or sprays), nasal administration, vaginal administration, rectal administration, topical administration, or transdermal administration, or by sustained release or extended release mechanisms. The compounds and compositions can be administered in unit dosage formulations containing conventional pharmaceutically acceptable carriers, excipients, adjuvants, and vehicles as desired. The compounds and compositions may be administered directly to a specific or affected organ or tissue. The compounds can be mixed with pharmaceutically acceptable carriers, excipients, adjuvants, and vehicles to form compositions appropriate for the desired route of administration. In some embodiments, the compounds can be mixed with one or both of an antigen and an adjuvant. In some embodiments, the antigen is a cancer antigen.

In certain embodiments disclosed herein, especially those embodiments where a formulation is used for injection or other parenteral administration, including the routes listed herein, but also including any other route of administration described herein (such as oral, enteric, gastric, etc.), the formulations and preparations used in the methods are sterile. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art. A “sterile” formulation is aseptic, or free or essentially free from all living microorganisms and their spores. Examples of methods of sterilization of pharmaceutical formulations include, but are not limited to, sterile filtration through sterile filtration membranes, exposure to radiation such as gamma radiation, and heat sterilization.

Oral administration is advantageous due to its ease of implementation and patient compliance. If a patient has difficulty swallowing, introduction of medicine via feeding tube, feeding syringe, or gastrostomy can be employed in order to accomplish enteric administration. The active compound, and, if present, other co-administered agents, can be enterally administered in any other pharmaceutically acceptable excipient suitable for formulation for administration via feeding tube, feeding syringe, or gastrostomy.

Intravenous administration also can be used advantageously, for delivery of the compounds or compositions to the bloodstream as quickly as possible and to circumvent the need for absorption from the gastrointestinal tract.

The compounds and compositions described for use herein can be administered in solid form, in liquid form, in aerosol form, or in the form of tablets, pills, caplets, capsules (such as hard gelatin capsules or soft elastic gelatin capsules), powder mixtures, granules, injectables, solutions, suppositories, enemas, colonic irrigations, emulsions, dispersions, food premixes, cachets, troches, lozenges, gums, ointments, cataplasms (poultices), pastes, powders, dressings, creams, patches, aerosols (e.g., nasal spray or inhalers), gels, suspensions (e.g., aqueous or non-aqueous liquid suspensions, oil-in-water emulsions or water-in-oil liquid emulsions), elixirs, or in other forms suitable for the route of administration. The compounds and compositions also can be administered in liposome formulations. The compounds also can be administered as prodrugs, where the prodrug undergoes transformation in the treated subject to a therapeutically effective form.

In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound also may contain other substances that have valuable therapeutic properties. Pharmaceutical formulations may be prepared by known pharmaceutical methods. Additional formulations and methods of administration are known in the art. Suitable formulations can be found, e.g., in Remington: The Science and Practice of Pharmacy, Lippincott Williams & Wilkins, 21st ed. (2005), which is incorporated herein by reference.

Injectable preparations, for example, sterile injectable aqueous or oleaginous suspensions, may be formulated according to methods known in the art using suitable dispersing or wetting agents and suspending agents. The sterile injectable preparation also may be a sterile injectable solution or suspension in a parenterally acceptable diluent or solvent, for example, as a solution in propylene glycol. Among the acceptable vehicles and solvents that may be employed are water, saline, Ringer's solution, and isotonic sodium chloride solution. In addition, sterile, fixed oils are conventionally employed as a solvent or suspending medium. For this purpose, any bland fixed oil may be employed including synthetic mono or di-glycerides. In addition, fatty acids such as oleic acid may be used in the preparation of injectables.

Solid dosage forms for oral administration may include capsules, tablets, pills, powders, and granules. In such solid dosage forms, the active compound may be admixed with at least one inert diluent such as sucrose, lactose, talc, or starch. Such dosage forms also may comprise additional excipient substances other than inert diluents, e.g., lubricating agents such as magnesium stearate. In the case of capsules, tablets, and pills, the dosage forms also may comprise buffering agents. Tablets and pills additionally can be prepared with enteric coatings. Acceptable excipients for gel capsules with a soft shell are, for instance, plant oils, wax, fats, semisolid and liquid poly-ols, and so on.

Liquid dosage forms for oral administration may include pharmaceutically acceptable emulsions, solutions, suspensions, syrups, and elixirs containing inert diluents commonly used in the art, such as water. Such compositions also may comprise additional agents, such as wetting agents, emulsifying and suspending agents, cyclodextrins, and sweetening, flavoring, and perfuming agents. Alternatively, the compound also may be administered in neat form if suitable.

The compounds and compositions also can be administered in the form of liposomes. As is known in the art, liposomes are generally derived from phospholipids or other lipid substances. Liposomes are formed by mono or multilamellar hydrated liquid crystals that are dispersed in an aqueous medium. Any physiologically acceptable and metabolizable lipid capable of forming liposomes can be used. The present compositions in liposome form can contain stabilizers, preservatives, excipients, and the like, in addition to a compound as disclosed herein. Useful lipids include the phospholipids and phosphatidyl cholines (lecithins), both natural and synthetic. Methods to form liposomes are known in the art. See, for example, Gregoriadis, G. Ed., Liposome Technology, Third Edition: Liposome Technology: Liposome Preparation and Related Techniques, CRC Press, Boca Raton, Fla. (2006); and Prescott, Ed., Methods in Cell Biology, Volume XIV, Academic Press, New York, N. W., p. 33 et seq (1976).

The amount of active ingredient that may be combined with the carrier materials to produce a single dosage form can vary depending upon the patient to whom the active ingredient is administered and the particular mode of administration. It will be understood, however, that the specific dose level for any particular patient will depend upon a variety of factors including the specific compound employed; the age, body weight, body area, body mass index (BMI), general health, sex, and diet of the patient; the time of administration and route of administration used; the rate of excretion; and the drug combination, if any, used. The compounds can be administered in a unit dosage formulation. The pharmaceutical unit dosage chosen is fabricated and administered to provide sufficient concentration of drug in the patient, subject, or individual.

Although the compounds for use as described herein can be administered as the sole active pharmaceutical agent, they also can be used in combination with one or more other agents. When additional active agents are used in combination with the compounds for use as described herein, the additional active agents may generally be employed in therapeutic amounts as indicated in the Physicians' Desk Reference (PDR) 71st Edition (2017), which is incorporated herein by reference, or such therapeutically useful amounts as would be known to one of ordinary skill in the art, or as are determined empirically for each patient.

Combinations of two or more of the compounds and compositions disclosed herein also can be used. The two or more compounds or compositions can be mixed together shortly before administration and administered together. The two or more compounds or compositions can be administered simultaneously, either by the same route of administration or by different routes of administration. The two or more compounds or compositions can be administered consecutively, either by the same route of administration or by different routes of administration. In one embodiment, a kit form can contain two or more compounds or compositions as individual compounds or compositions, with printed or electronic instructions for administration either as a mixture of compounds or compositions, as separate compounds or compositions administered simultaneously, or as separate compounds or compositions administered consecutively. Where three or more compounds or compositions are administered, they can be administered as a mixture of compounds or compositions, as separate compounds or compositions administered simultaneously, as separate compounds or compositions administered consecutively, as separate compounds or compositions where two or more may be administered simultaneously with the remainder administered consecutively before or after the simultaneous administration, or any other possible combination of mixed administration, simultaneous administration, and consecutive administration.

A compound as disclosed herein may in one aspect be in a purified form and compositions comprising a compound in purified forms are disclosed herein. Compositions comprising a compound as disclosed herein or a salt thereof are provided, such as compositions of substantially pure compounds. In some embodiments, a composition containing a compound as disclosed herein or a salt thereof is in substantially pure form. In one variation, “substantially pure” intends a composition that contains no more than 35% impurity, wherein the impurity denotes a compound other than the compound (or compounds, if combinations of compounds are used) to be administered in the composition, or a salt or solvate of the compound (or compounds, if combinations are used). The weight of any added vehicle, carrier, or excipient is excluded from such a calculation, and the added vehicle, carrier, or excipient is not considered as an impurity. For example, a composition of a substantially pure compound selected from a compound of Table 1 refers to a composition that contains no more than 35% impurity, wherein the impurity denotes a compound other than the compound or a salt or solvate thereof. In one variation, a composition of substantially pure compound or a salt or solvate thereof is provided wherein the composition contains no more than 25% impurity. In another variation, a composition of substantially pure compound or a salt or solvate thereof is provided wherein the composition contains no more than 20% impurity. In still another variation, a composition of substantially pure compound or a salt or solvate thereof is provided wherein the composition contains no more than 10% impurity. In a further variation, a composition of substantially pure compound or a salt or solvate thereof is provided wherein the composition contains no more than 5% impurity. In another variation, a composition of substantially pure compound or a salt or solvate thereof is provided wherein the composition contains no more than 3% impurity. In still another variation, a composition of substantially pure compound or a salt or solvate thereof is provided wherein the composition contains no more than 1% impurity. In a further variation, a composition of substantially pure compound or a salt or solvate thereof is provided wherein the composition contains no more than 0.5% impurity. In yet other variations, a composition of substantially pure compound means that the composition contains no more than 15%, no more than 10%, no more than 5%, no more than 3%, or no more than 1% impurity. An impurity may be the compound in a stereochemical form different from the desired stereochemical form. For instance, a composition of substantially pure (S) compound means that the composition contains no more than 15%, no more than 10%, no more than 5%, no more than 3%, or no more than 1% of the (R) form of the compound.

In some aspects, provided herein are compositions comprising a cell population containing a modified immune cell such as those described herein or produced by the methods disclosed herein. In some embodiments, the composition comprises a cell population containing a modified immune cell that has been in contact or is in contact with a Cbl-b inhibitor described herein or a composition thereof. In some embodiments, the modified immune cell has been or is in contact with an anti-CD3 antibody alone. In some embodiments, the modified immune cell has been or is in contact with an anti-CD3 antibody in combination with an anti-CD28 antibody. The provided compositions comprising a cell population containing a modified immune cell described herein may further comprise a pharmaceutical acceptable excipient.

In some aspects, also provided herein is a cell culture composition comprising a cell population containing an immune cell and a Cbl-b inhibitor described herein. In some embodiments, the immune cell is a cell selected from the group consisting of: a hematopoietic cell, a multipotent stem cell, a myeloid progenitor cell, a lymphoid progenitor cell, a T-cell, a B cell, and a NK cell. In some embodiments, the cell culture composition further comprises an anti-CD3 antibody. In some embodiments, the cell culture composition further comprises an anti-CD3 antibody in combination with an anti-CD28 antibody. Methods for culturing cell compositions containing immune cells are well known in the art and are contemplated herein.

A modified immune cell or compositions as described herein, e.g., a composition comprising a cell population containing the modified immune cell or a pharmaceutical composition, can be provided in a suitable container. Suitable containers include, for example, bottles, vials (e.g., dual chamber vials), syringes (e.g., single or dual chamber syringes), bags (e.g., an intravenous bag) and tubes (e.g., test tubes). The container may be formed from a variety of materials such as glass or plastic.

In some embodiments, a composition comprising a cell population containing a modified immune cell as described herein (e.g., a cell culture composition) is provided in a culture vessel. A culture vessel as provided herein includes, but is not limited to, a tube (e.g., a test tube), a dish (e.g., a tissue culture dish), a bag, a multiwell plate (e.g., a 6-well tissue culture plate) and a flask (e.g., a cell culture flask).

Also provided are the compositions as described herein for any use described herein. In some embodiments, the compositions as described herein are for preparation of a medicament for treating or preventing a disease or condition associated with Cbl-b activity. In some embodiments, the compositions as described herein are for preparation of a medicament for treating cancer.

### V. Articles of Manufacture or Kits

Also provided are articles of manufacture comprising any of the compounds, pharmaceutical compositions, cells, modified immune cells, cell populations, cell compositions, cell cultures, or cell culture compositions described herein. The articles of manufacture include suitable containers or packaging materials for the compounds, pharmaceutical compositions, cells, modified immune cells, cell populations, cell compositions, cell cultures, or cell culture compositions. Examples of a suitable container include, but are not limited to, a bottle, a vial, a syringe, an intravenous bag or a tube. For cells, modified immune cells, cell populations, cell compositions, cell cultures, or cell culture compositions, a suitable container can be a culture vessel, including, but not limited to, a tube, a dish, a bag, a multiwell plate, or a flask.

Also provided are kits comprising any of the compounds, pharmaceutical compositions, cells, modified immune cells, cell populations, cell compositions, cell cultures, or cell culture compositions described herein. The kits can contain the compounds, pharmaceutical compositions, cells, modified immune cells, cell populations, cell compositions, cell cultures, or cell culture compositions in suitable containers or packaging materials, including, but not limited to, a bottle, a vial, a syringe, an intravenous bag or a tube. The kits can contain cells, modified immune cells, cell populations, cell compositions, cell cultures, or cell culture compositions in a culture vessel, including, but not limited to, a tube, a dish, a bag, a multiwell plate, or a flask. The kits can comprise the compounds, pharmaceutical compositions, cells, modified immune cells, cell populations, cell compositions, cell cultures, or cell culture compositions for administration to an individual in single-dose form or in multiple-dose form. The kits can further comprise instructions or a label for administering the compounds, pharmaceutical compositions, cells, modified immune cells, cell populations, cell compositions, cell cultures, or cell culture compositions to an individual according to any of the methods disclosed herein. The kits can further comprise equipment for administering the compounds, pharmaceutical compositions, cells, modified immune cells, cell populations, cell compositions, cell cultures, or cell culture compositions to an individual, including, but not limited to, needles, syringes, tubing, or intravenous bags. The kits can further comprise instructions for producing any of the compounds, pharmaceutical compositions, cells, modified immune cells, cell populations, cell compositions, cell cultures, or cell culture compositions disclosed herein.

The disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXEMPLARY EMBODIMENTS

The invention is further described by the following embodiments. The features of each of the embodiments are combinable with any of the other embodiments where appropriate and practical.

Embodiment 1. A compound of Formula (I-A):

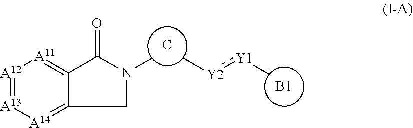

or a tautomer thereof, or a pharmaceutically acceptable salt of any of the foregoing,

wherein

A¹¹ is CR¹¹ or N,

A¹² is CR¹² or N,

A¹³ is CR¹³ or N, and

A¹⁴ is CR¹⁴ or N,

wherein no more than two of A¹¹, A¹², A¹³, and A¹⁴ are N;

R¹¹, R¹², R¹³, and R¹⁴ are independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl

—NR^{j}R^{k} where R^{j} and R^{k} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{k} can be additionally chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{j} and R^{k} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a —C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{l}R^{m}, —O—(C₁-C₄ alkylene)-NR^{l}R^{m}, —C(═O)NR^{l}R^{m}, —(C₁-C₄ alkylene)-C(═O)NR^{l}R^{m}, or —O—(C₁-C₄ alkylene)-C(═O)NR^{l}R^{m}, wherein R^{l} and R^{m} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{l} and R^{m} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH,

—S(═O)₂—C₁-C₈ alkyl,

—SF₅, and

—S(═O)₂NRⁿR^{o} wherein Rⁿ and R^{o} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or Rⁿ and R^{o} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

Ring C,

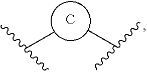

is selected from the group consisting of:

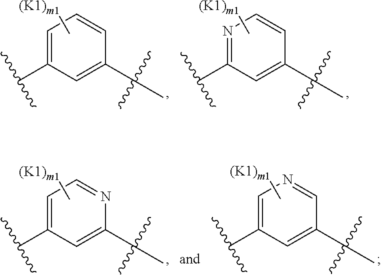

each K1 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —C₁-C₈ alkyl, —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO-(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, Ci-C's hydroxyalkyl, —CN, or —O—C₁-C₈ alkyl;

or two vicinal K1 groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —C₁-C₈ haloalkyl, —O—C₁-C₈ alkyl, and —NR^{g1}R^{h1}, where R^{g1} and R^{h1} are independently H or C₁-C₈ alkyl;

m1 is 0, 1, or 2;

[symbol] is a single bond or a double bond,

wherein when [symbol] is a single bond, Y1 is C(R¹⁹)(R²⁰), S, or O; and Y2 is C(R¹⁷)(R¹⁸), and

when [symbol] is a double bond, Y1 is C(R¹⁹); and Y2 is C(R¹⁸),

R¹⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R¹⁸ is selected from the group consisting of H, F, —OH, C₁-C₈ alkyl optionally substituted with —OH, halogen or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl, or

when Y1 is C(R¹⁹)(R²⁰) or C(R¹⁹), R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl, or

R¹⁷ and R¹⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or C₁-C₈ alkylene-OH;

R¹⁹ and R²⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R¹⁹ can be taken together with R¹⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; or

R¹⁹ and R²⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and

Ring B1,

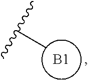

is a five-membered heteroaryl ring containing at least one N, O, or S ring atom, wherein Ring B1 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 2. The compound or tautomer thereof, or pharmaceutically acceptable salt of the compound or tautomer, of embodiment 1, wherein the compound of Formula (I-A) is a compound of Formula (I):

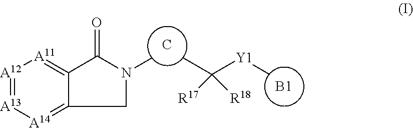

or a tautomer thereof, or a pharmaceutically acceptable salt of any of the foregoing,

wherein

A¹¹ is CR¹¹ or N,

A¹² is CR¹² or N,

A¹³ is CR¹³ or N, and

A¹⁴ is CR¹⁴ or N,

wherein no more than two of A¹¹, A¹², A¹³, and A¹⁴ are N;

R¹¹, R¹², R¹³, and R¹⁴ are independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{j}R^{k} where R^{j} and R^{k} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{k} can be additionally chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{j} and R^{k} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{l}R^{m}, —O—(C₁-C₄ alkylene)-NR^{l}R^{m}. —C(═O)NR^{l}R^{m}, —(C₁-C₄ alkylene)-C(═O)NR^{l}R^{m}, or —O—(C₁-C₄ alkylene)-C(═O)NR^{l}R^{m}, wherein R^{l} and R^{m} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{l} and R^{m} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH,

—S(═O)₂—C₁-C₈ alkyl,

—SF₅, and

—S(═O)₂NRⁿR^{o} wherein Rⁿ and R^{o} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or Rⁿ and R^{o} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

Ring C,

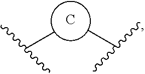

is selected from the group consisting of:

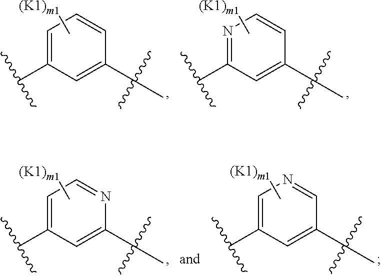

each K1 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, and four- to eight-membered heterocyclyl optionally substituted with —OH or C₁-C₄ alkyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K1 groups are taken together with the atoms to which they are attached to form a three- to six-membered carbocyclic or heterocyclic ring, a phenyl ring, or a five- to six-membered heteroaryl ring, wherein the ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —C₁-C₈ haloalkyl, —O—C₁-C₈ alkyl, and —NR^{g1}R^{h1}, where R^{g1} and R^{h1} are independently H or C₁-C₈ alkyl;

m1 is 0, 1, or 2;

R¹⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R¹⁸ is selected from the group consisting of H, F, —OH, C₁-C₈ alkyl optionally substituted with —OH, halogen or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl, or

when Y1 is C(R¹⁹)(R²⁰), R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl, or

R¹⁷ and R¹⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or C₁-C₈ alkylene-OH;

Y1 is C(R¹⁹)(R²⁰) or S;

R¹⁹ and R²⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R¹⁹ can be taken together with R¹⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; or

R¹⁹ and R²⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl;

Ring B1,

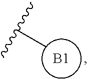

is a five-membered heteroaryl ring containing at least one N, O, or S ring atom, wherein Ring B1 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 3. The compound or tautomer thereof, or pharmaceutically acceptable salt of the compound or tautomer, of embodiment 1 or embodiment 2, wherein

A¹¹ is CR¹¹ or N,

A¹² is CR¹² or N,

A¹³ is CR¹³ or N, and

A¹⁴ is CR¹⁴ or N,

wherein no more than two of A¹¹, A¹², A¹³, and A¹⁴ are N;

R¹¹, R¹², R¹³, and R¹⁴ are independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{j}R^{k} where R^{j} and R^{k} are independently H, C₁-C₈ alkyl, C₁-C₈ haloalkyl, or C₃-C₈ cycloalkyl, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{k} can be additionally chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{j} and R^{k} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, or I, —(C₁-C₄ alkylene)-NR^{l}R^{m}, —O—(C₁-C₄ alkylene)-NR^{l}R^{m}, —C(═O)NR^{l}R^{m}, —(C₁-C₄ alkylene)-C(═O)NR^{l}R^{m}, or —O—(C₁-C₄ alkylene)-C(═O)NR^{l}R^{m}, wherein R^{l} and R^{m} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{l} and R^{m} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH,

—S(═O)₂—C₁-C₈ alkyl,

—SF₅, and

—S(═O)₂NRⁿR^{o} wherein Rⁿ and R^{o} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or Rⁿ and R^{o} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

Ring C,

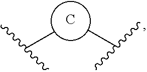

is selected from the group consisting of:

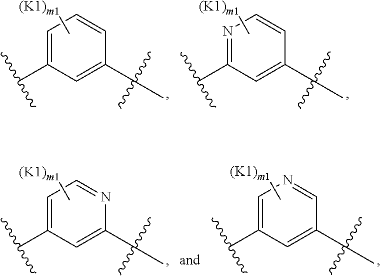

each K1 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl,

—O—C₁-C₈ haloalkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K1 groups are taken together with the atoms to which they are attached to form a three- to six-membered carbocyclic or heterocyclic ring, a phenyl ring, or a five- to six-membered heteroaryl ring, wherein the ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —C₁-C₈ haloalkyl, —O—C₁-C₈ alkyl, and —NR^{g1}R^{h1}, where R^{g1} and R^{h1} are independently H or C₁-C₈ alkyl;

m1 is 0, 1, or 2;

R¹⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R¹⁸ is selected from the group consisting of H, F, —OH, C₁-C₈ alkyl optionally substituted with —OH, halogen or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl, or

when Y1 is C(R¹⁹)(R²⁰), R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl, or

R¹⁷ and R¹⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and

Y is C(R¹⁹)(R²⁰) or S;

R¹⁹ and R²⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R¹⁹ can be taken together with R¹⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; and

Ring B1,

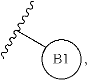

is a five-membered heteroaryl ring containing at least one N, O, or S ring atom, wherein Ring B1 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 4. The compound or tautomer thereof, or pharmaceutically acceptable salt of the compound or tautomer, of any one of embodiments 1-3, wherein A¹¹ is CR¹¹, A¹² is CR¹², A¹³ is CR¹³, and A¹⁴ is CR¹⁴.

Embodiment 5. The compound or tautomer thereof, or pharmaceutically acceptable salt of the compound or tautomer, of any one of embodiments 1-3, wherein A¹¹ is CR¹¹, A¹² is N, A¹³ is CR¹³, and A¹⁴ is CR¹⁴.

Embodiment 6. The compound or tautomer thereof, or pharmaceutically acceptable salt of the compound or tautomer, of any one of embodiments 1-3, wherein A¹¹ is CR¹¹, A¹² is CR¹², A¹³ is N, and A¹⁴ is CR¹⁴.

Embodiment 7. The compound or tautomer thereof, or pharmaceutically acceptable salt of the compound or tautomer, of any one of embodiments 1-3, wherein A¹¹ is CR¹¹, A¹² is N, A¹³ is N, and A¹⁴ is CR¹⁴.

Embodiment 8. The compound or tautomer thereof, or pharmaceutically acceptable salt of the compound or tautomer, of any one of embodiments 1-3, wherein:

a) A¹¹ is N, A¹² is CR¹², A¹³ is CR¹³, and A¹⁴ is CR¹⁴;

b) A¹¹ is CR¹¹, A¹² is CR¹², A¹³ is CR¹³, and A¹⁴ is N;

c) A¹¹ is N, A¹² is N, A¹³ is CR¹³, and A¹⁴ is CR¹⁴;

d) A¹¹ is N, A¹² is CR¹², A¹³ is N, and A¹⁴ is CR¹⁴;

e) A¹¹ is N, A¹² is CR¹², A¹³ is CR¹³, and A¹⁴ is N;

f) A¹¹ is CR¹¹, A¹² is N, A¹³ is CR¹³, and A¹⁴ is N; or

g) A¹¹ is CR¹¹, A¹² is CR¹², A¹³ is N, and A¹⁴ is N.

Embodiment 9. The compound or tautomer thereof, or pharmaceutically acceptable salt of the compound or tautomer, of any one of embodiments 1-8, wherein Ring C,

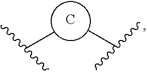

is

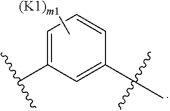

Embodiment 10. The compound or tautomer thereof, or pharmaceutically acceptable salt of the compound or tautomer, of any one of embodiments 1-9, wherein at least one of R¹¹, R¹², R¹³, and R¹⁴ is selected from the group consisting of: a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 11. The compound or tautomer thereof, or pharmaceutically acceptable salt of the compound or tautomer, of any one of embodiments 1-9, wherein at least one of R¹¹, R¹², R¹³, and R¹⁴ is selected from the group consisting of: —C₁-C₈ haloalkyl-OH, —C₁-C₈ haloalkyl-COOH, —CO(C₁-C₈ alkyl), a nine-membered heterocyclic ring, —CH(C₁-C₈ alkyl) (four-to eight-membered heterocyclic ring), a —CH(C₁-C₈ haloalkyl)-(four- to eight-membered heterocyclic ring), a —CH(OH)-(C₆-C₁₄ aryl ring), and a —O-(four- to eight-membered heterocyclic ring), wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring.

Embodiment 12. The compound or tautomer thereof, or pharmaceutically acceptable salt of the compound or tautomer, of embodiment 10, wherein R¹² is a —(C₁-C₄ alkylene)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 13. The compound or tautomer thereof, or pharmaceutically acceptable salt of the compound or tautomer, of embodiment 12, wherein R¹² is a —(C₁-C₂ alkylene)-(four-membered heterocyclic ring), a —(C₁-C₂ alkylene)-(five-membered heterocyclic ring), or a —(C₁-C₂ alkylene)-(six-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 14. The compound or tautomer thereof, or pharmaceutically acceptable salt of the compound or tautomer, of embodiment 10, wherein R¹² is a —CH₂-(four- to eight-membered heterocyclic ring) or a —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 15. The compound or tautomer thereof, or pharmaceutically acceptable salt of the compound or tautomer, of embodiment 10, wherein R¹² is —CH₂-(pyrrolidinyl) or —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 16. The compound or tautomer thereof, or pharmaceutically acceptable salt of the compound or tautomer, of embodiment 1, wherein at least one of R¹³, R¹², R¹³, and R¹⁴ is selected from the group consisting of: —C₁-C₈ haloalkyl-OH, —C₁-C₈ haloalkyl-COOH, —CO(C₁-C₈ alkyl), a nine-membered heterocyclic ring, —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), and a —O-(four- to eight-membered heterocyclic ring), wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —Cᵣ C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring.

Embodiment 17. The compound or tautomer thereof, or pharmaceutically acceptable salt of the compound or tautomer, of embodiment 16, wherein R¹² is a —(C₁-C₄ alkylene)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 18. The compound or tautomer thereof, or pharmaceutically acceptable salt of the compound or tautomer, of embodiment 17, wherein R¹² is a —(C₁-C₂ alkylene)-(four-membered heterocyclic ring), a —(C₁-C₂ alkylene)-(five-membered heterocyclic ring), or a —(C₁-C₂ alkylene)-(six-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 19. The compound or tautomer thereof, or pharmaceutically acceptable salt of the compound or tautomer, of embodiment 16, wherein R¹² is a —CH₂-(four- to eight-membered heterocyclic ring) or a —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 20. The compound or tautomer thereof, or pharmaceutically acceptable salt of the compound or tautomer, of embodiment 16, wherein R¹² is —CH₂-(pyrrolidinyl) or —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 21. The compound or tautomer thereof, or pharmaceutically acceptable salt of the compound or tautomer, of embodiment 10, wherein R¹² is —CH₂-(azetidinyl) or —CH(CH₃)-azetidinyl, wherein the azetidinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 22. The compound or tautomer thereof, or pharmaceutically acceptable salt of the compound or tautomer, of any one of embodiments 1-21, wherein R¹⁴ is selected from the group consisting of H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl.

Embodiment 23. The compound or tautomer thereof, or pharmaceutically acceptable salt of the compound or tautomer, of any one of embodiments 1-21, wherein R¹⁴ is CF₃.

Embodiment 24. The compound or tautomer thereof, or pharmaceutically acceptable salt of the compound or tautomer, of any one of embodiments 1-23, wherein R¹¹, R¹², R¹³, and R¹⁴ are independently selected from the group consisting of

H, F, Cl, Br, I, —CN, C₁-C₈ alkyl, —O—C₁-C₈ alkyl, —C₁-C₈ alkyl-OH, C₁-C₈ haloalkyl, C₃-C₆ cycloalkyl, —S(═O)₂—C₁-C₈ alkyl, —(C₁-C₄ alkylene)-NR^{y}R^{z}, and —C(═O)NR^{y}R^{z},

wherein R^{y} and R^{z} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

Embodiment 25. The compound or tautomer thereof, or pharmaceutically acceptable salt of the compound or tautomer, of any one of embodiments 1-23, wherein

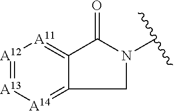

is selected from the group consisting of,

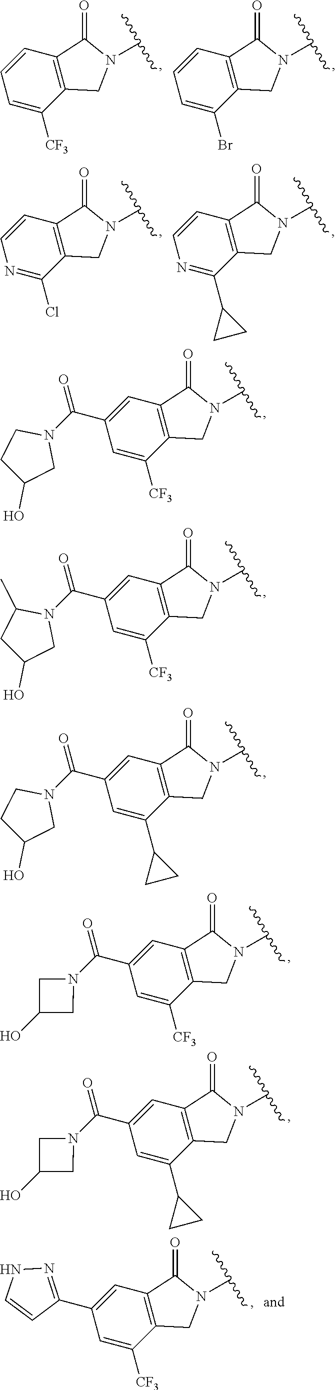

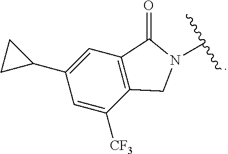 [SMILES: *N1Cc2c(cc(C3CC3)cc2C(F)(F)F)C1=O]

Embodiment 26. The compound or tautomer thereof, or pharmaceutically acceptable salt of the compound or tautomer, of any one of embodiments 1-23, wherein

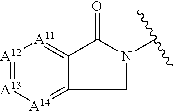

is selected from the group consisting of

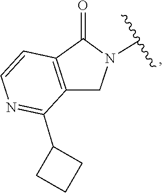 [SMILES: *N1Cc2c(ccnc2C2CCC2)C1=O]

Embodiment 27. The compound or tautomer thereof, or pharmaceutically acceptable salt of the compound or tautomer, of any one of embodiments 1-23, wherein

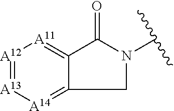

is selected from the group consisting of

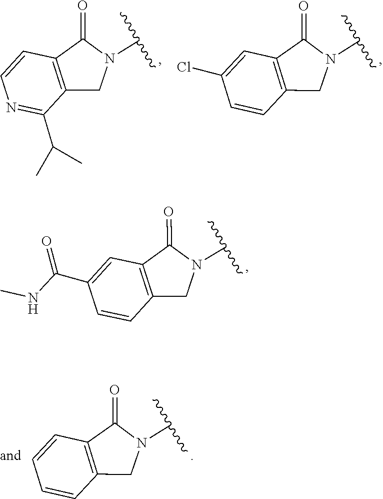 [SMILES: *N1Cc2c(ccnc2C(C)C)C1=O ; *N1Cc2ccc(Cl)cc2C1=O ; *N1Cc2ccc(C(=O)NC)cc2C1=O ; *N1Cc2ccccc2C1=O]

Embodiment 28. The compound or tautomer thereof, or pharmaceutically acceptable salt of the compound or tautomer, of any one of embodiments 1-23, wherein

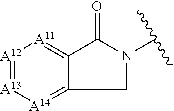

is selected from the group consisting of

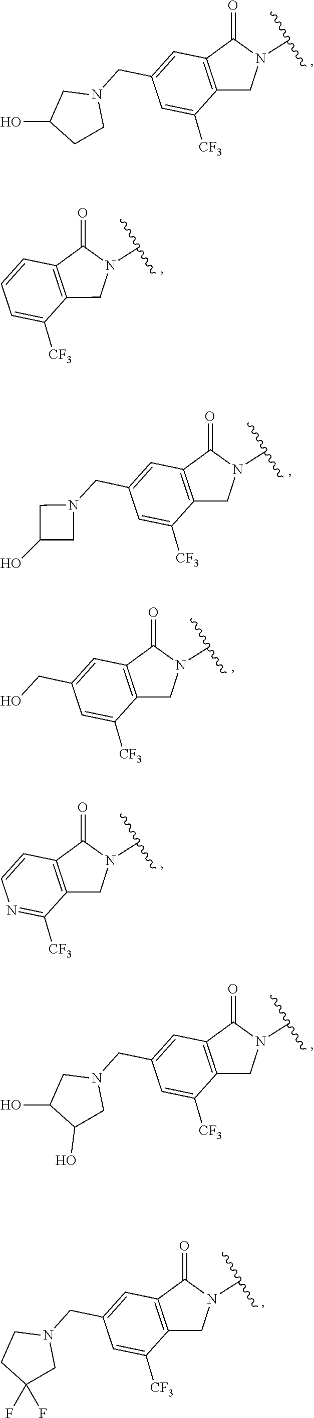

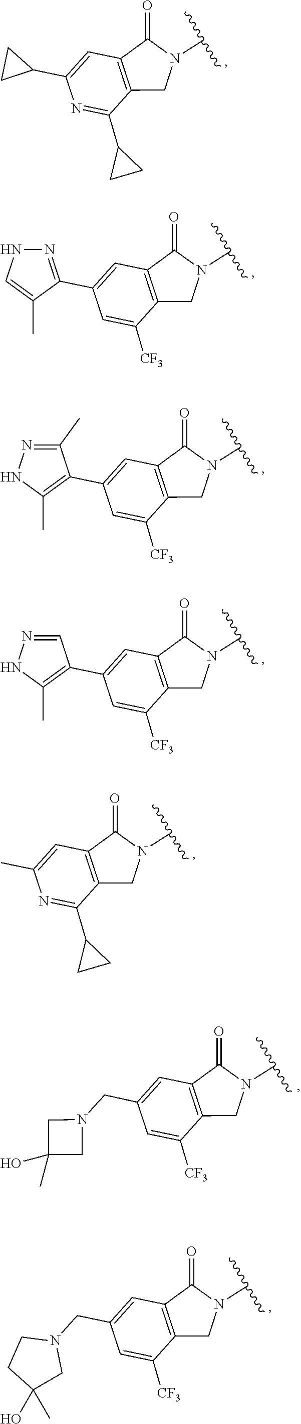

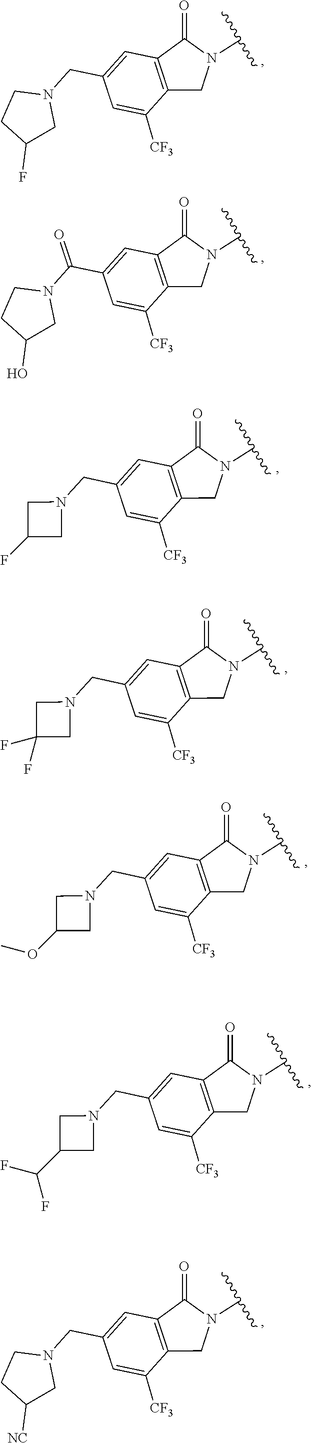

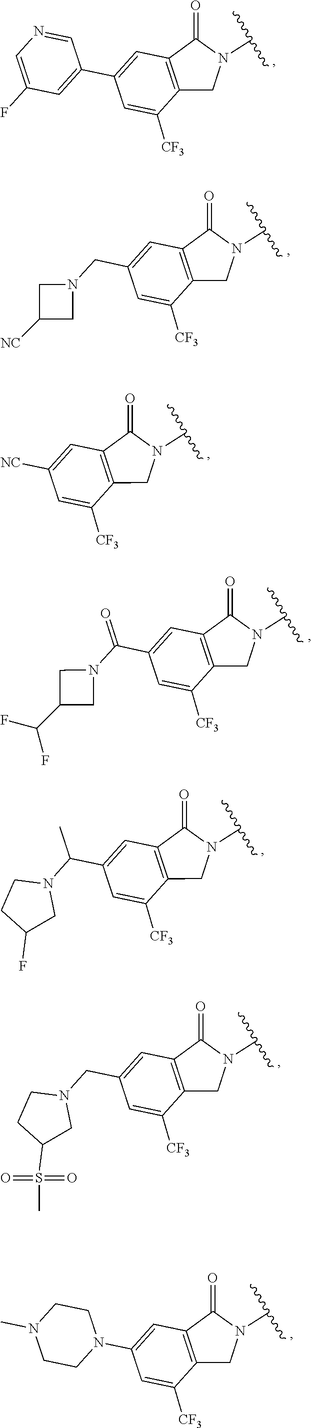

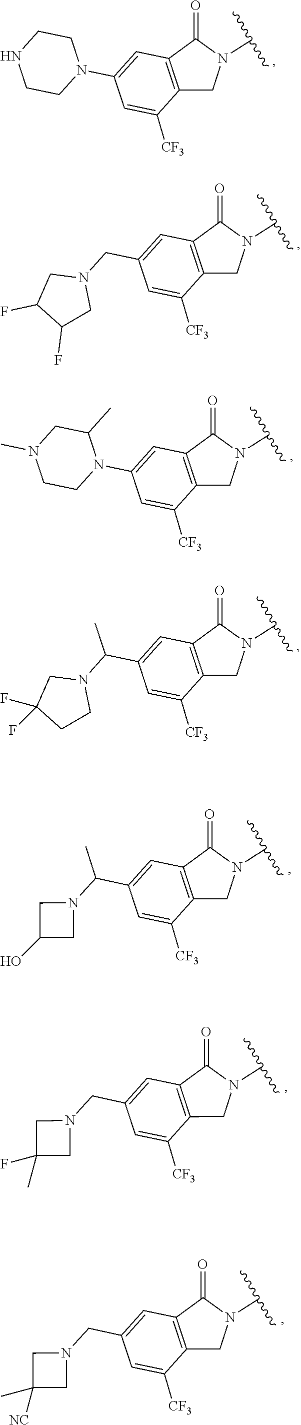

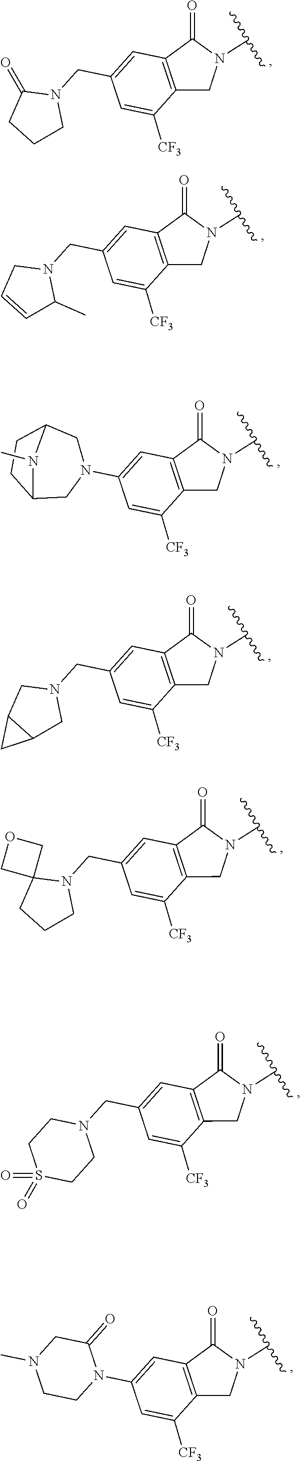

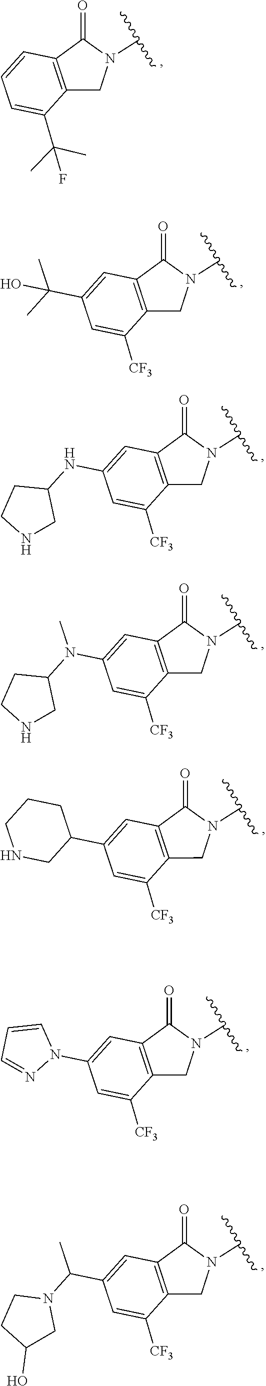

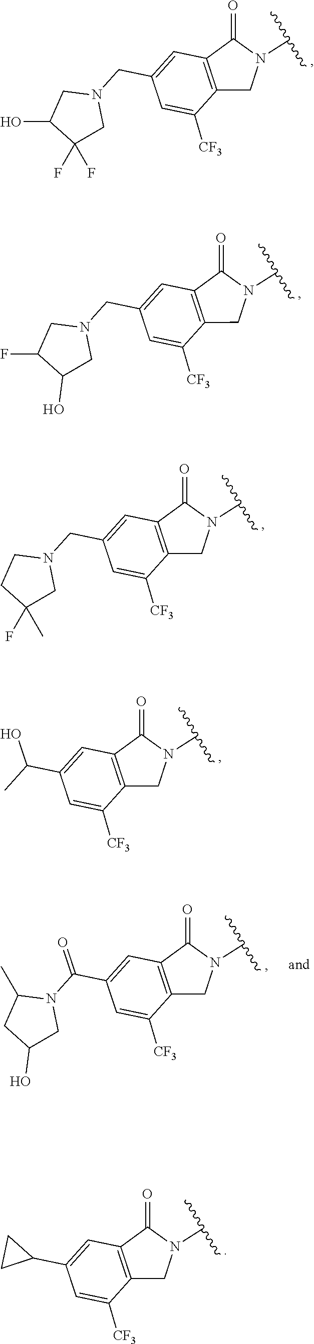 [SMILES: *N1Cc2c(cc(CN3CC(O)C(F)(F)C3)cc2C(F)(F)F)C1=O ; *N1Cc2c(cc(CN3CC(O)C(F)C3)cc2C(F)(F)F)C1=O ; *N1Cc2c(cc(CN3CCC(C)(F)C3)cc2C(F)(F)F)C1=O ; *N1Cc2c(cc(C(C)O)cc2C(F)(F)F)C1=O ; *N1Cc2c(cc(C(=O)N3CC(O)CC3C)cc2C(F)(F)F)C1=O ; *N1Cc2c(cc(C3CC3)cc2C(F)(F)F)C1=O]

Embodiment 29. The compound or tautomer thereof, or pharmaceutically acceptable salt of the compound or tautomer, of any one of embodiments 1-23, wherein

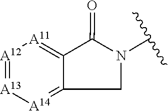

is selected from the group consisting of

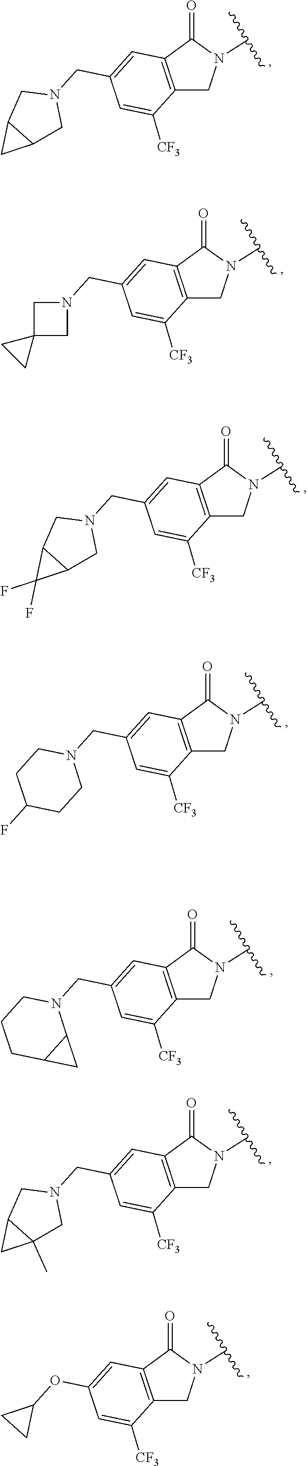

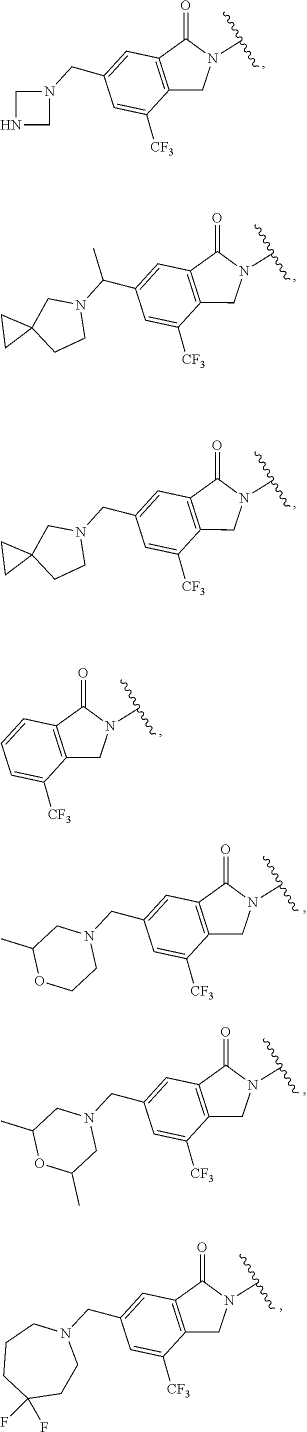

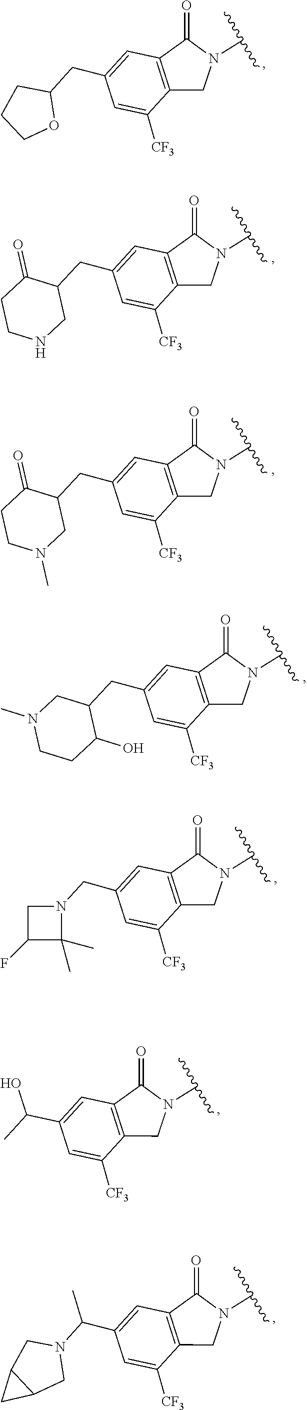

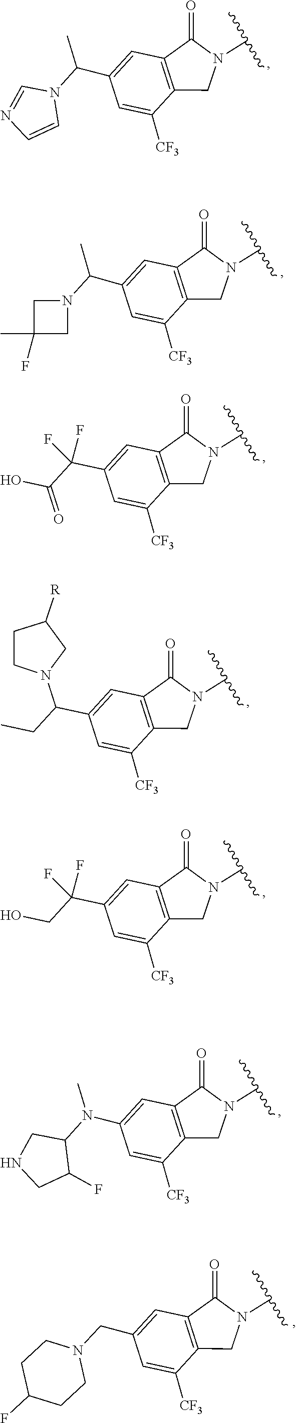

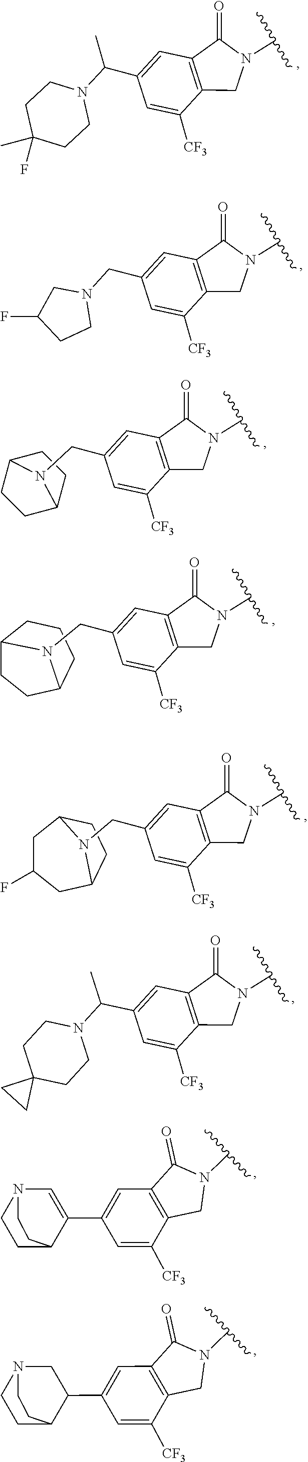

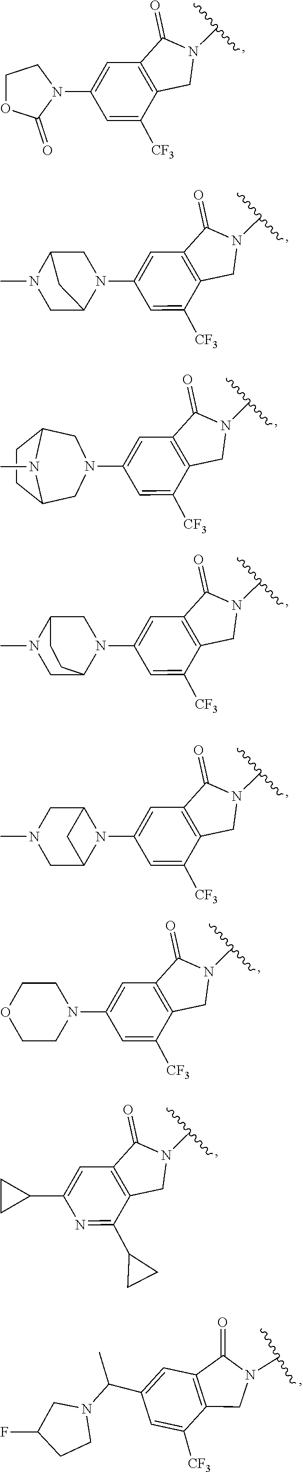

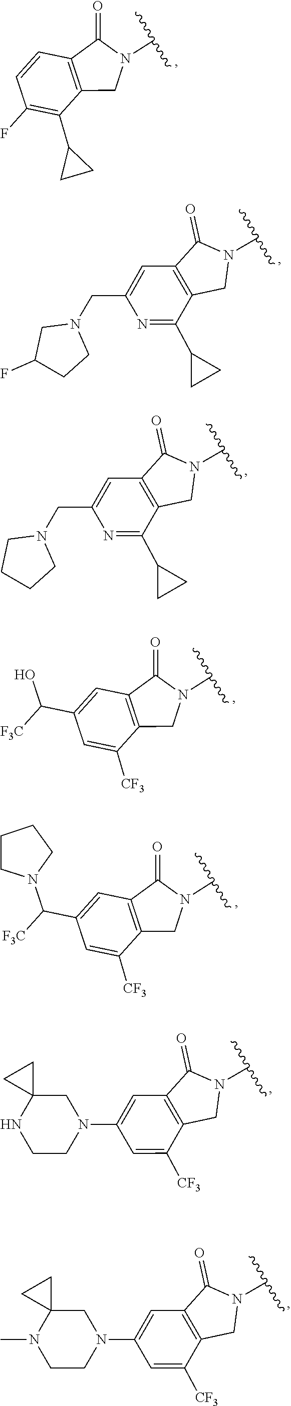

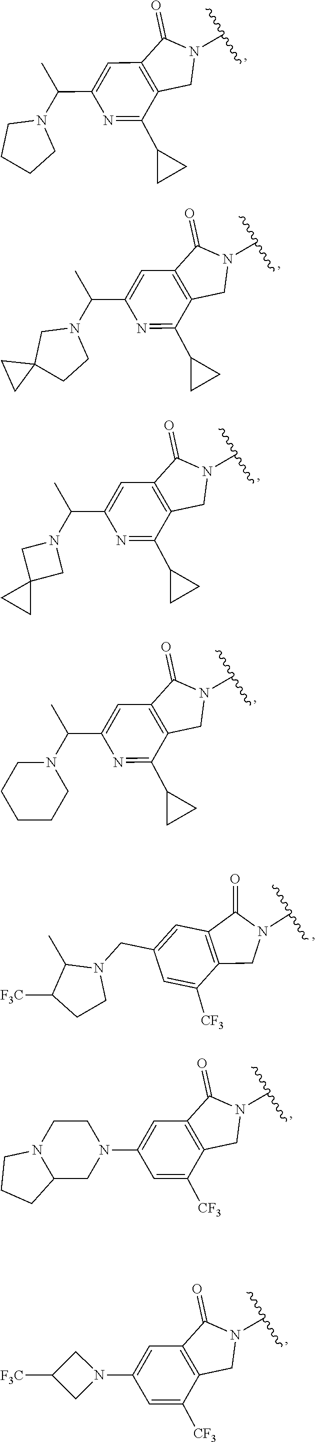

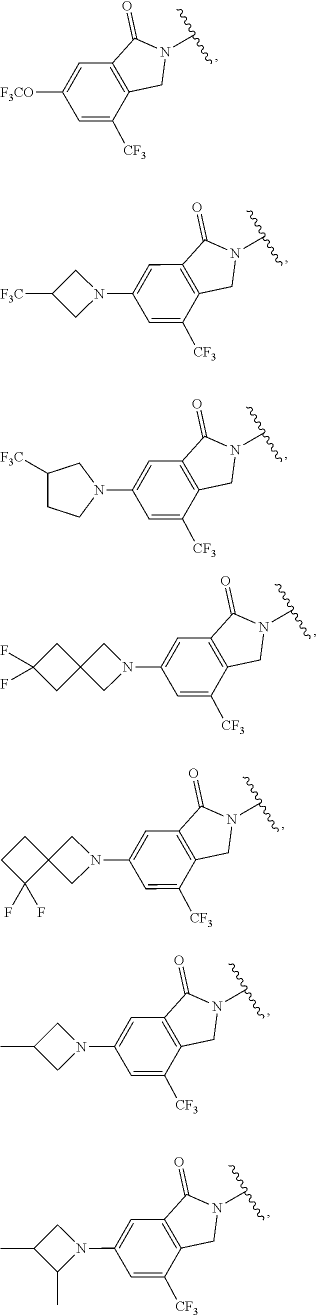

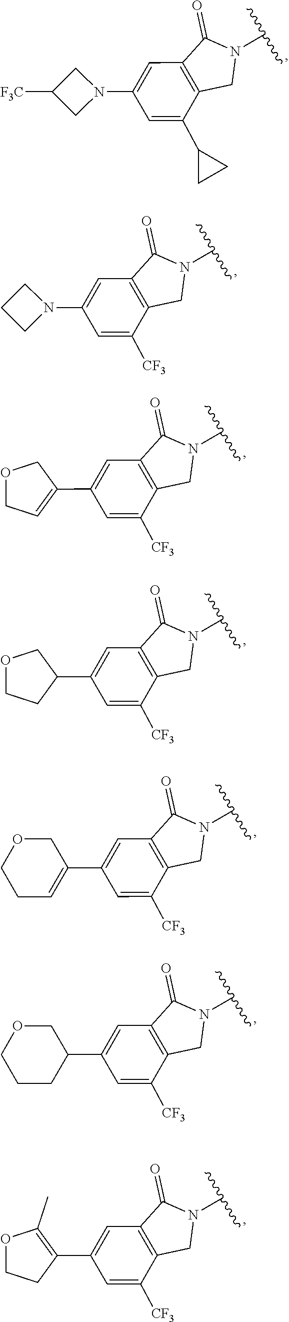

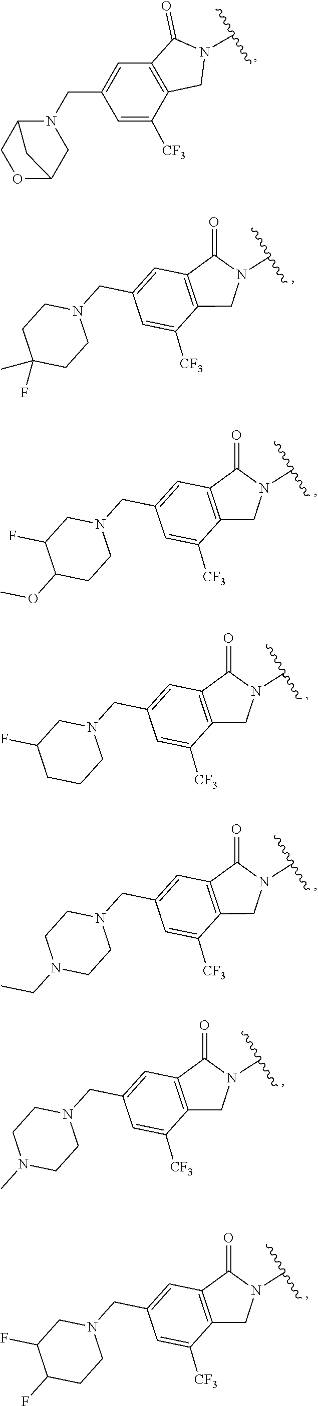

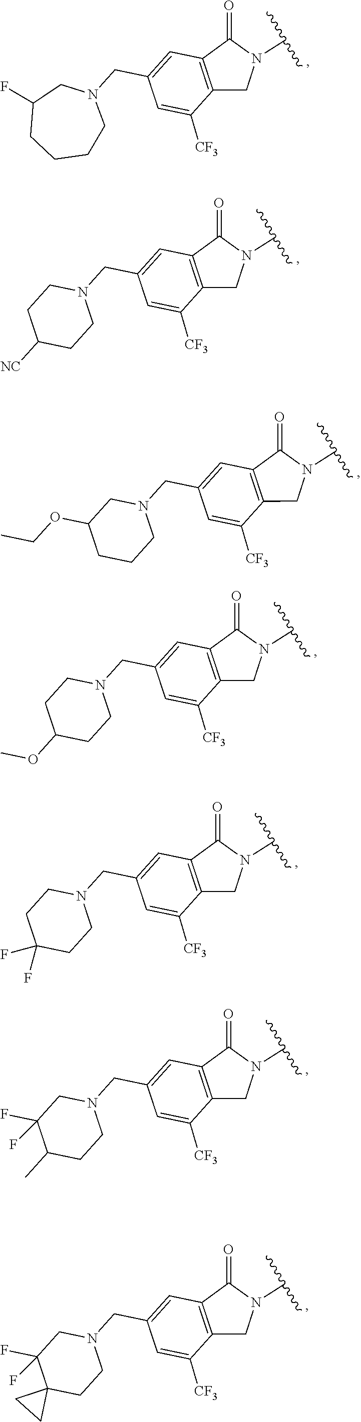

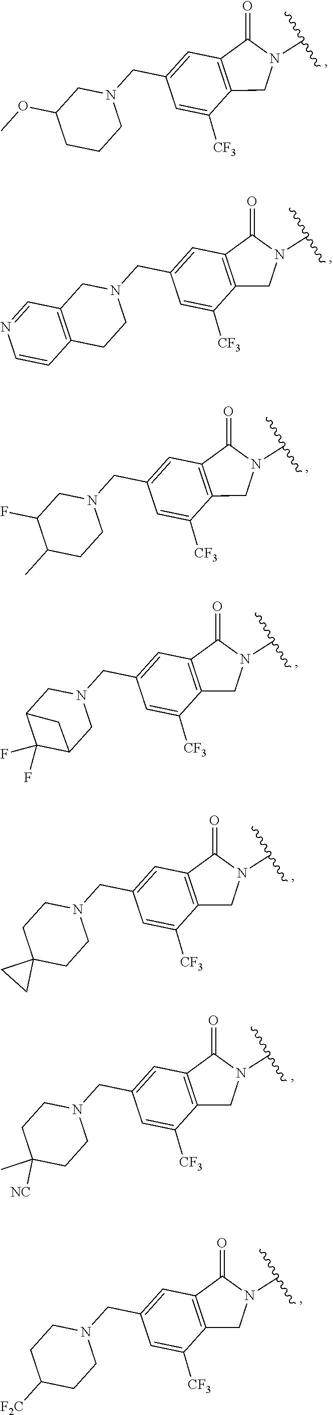

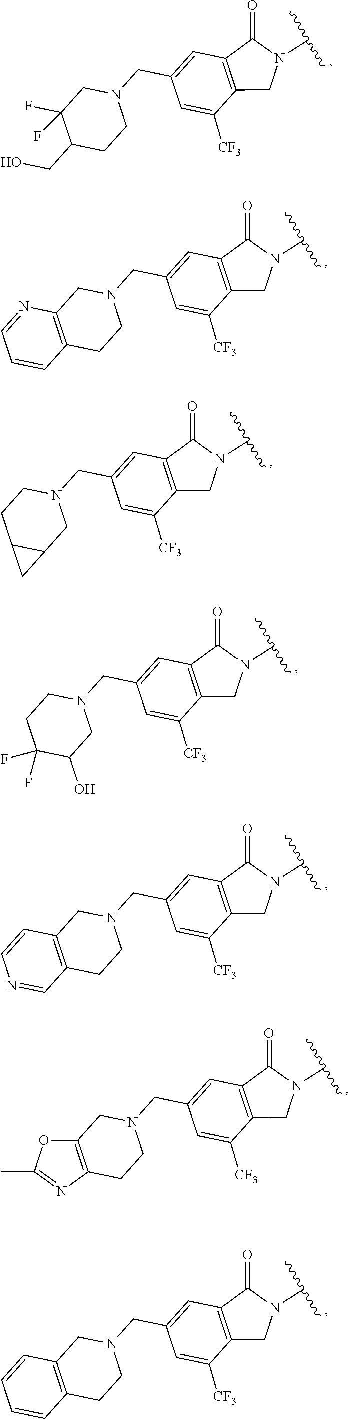

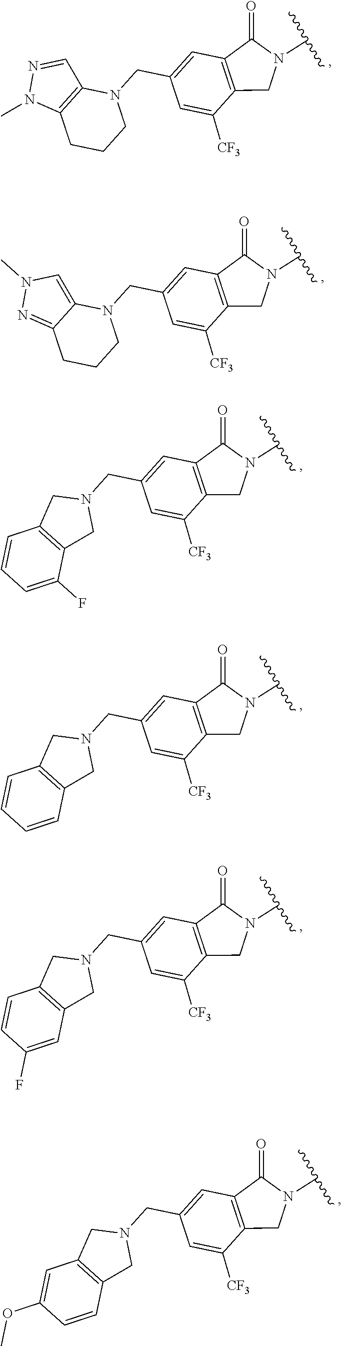 [SMILES: *N1Cc2c(cc(CN3CCCc4c3cnn4C)cc2C(F)(F)F)C1=O ; *N1Cc2c(cc(CN3CCCc4nn(C)cc43)cc2C(F)(F)F)C1=O ; *N1Cc2c(cc(CN3Cc4cccc(F)c4C3)cc2C(F)(F)F)C1=O ; *N1Cc2c(cc(CN3Cc4ccccc4C3)cc2C(F)(F)F)C1=O ; *N1Cc2c(cc(CN3Cc4ccc(F)cc4C3)cc2C(F)(F)F)C1=O ; *N1Cc2c(cc(CN3Cc4ccc(OC)cc4C3)cc2C(F)(F)F)C1=O]

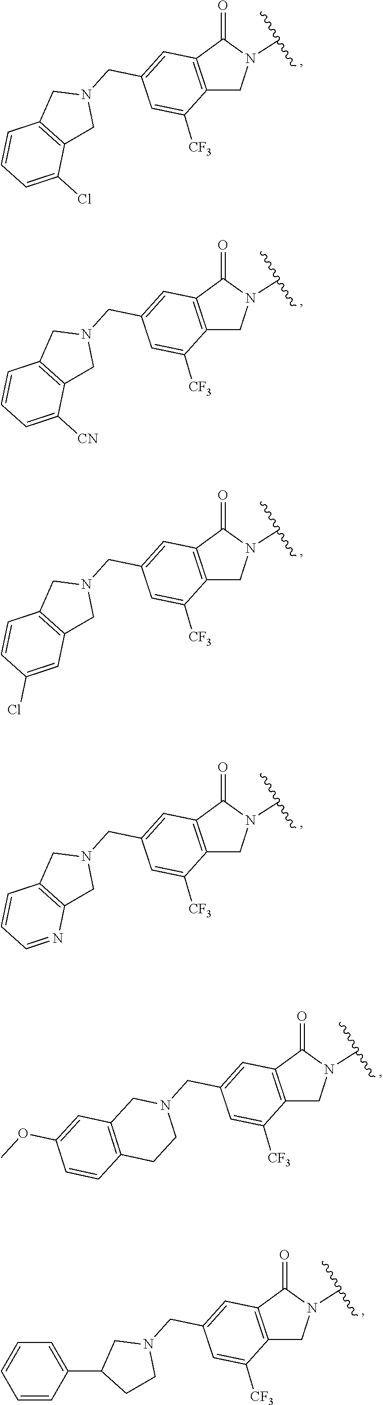 [SMILES: *N1Cc2c(cc(CN3Cc4cccc(Cl)c4C3)cc2C(F)(F)F)C1=O ; *N1Cc2c(cc(CN3Cc4cccc(C#N)c4C3)cc2C(F)(F)F)C1=O ; *N1Cc2c(cc(CN3Cc4ccc(Cl)cc4C3)cc2C(F)(F)F)C1=O ; *N1Cc2c(cc(CN3Cc4cccnc4C3)cc2C(F)(F)F)C1=O ; *N1Cc2c(cc(CN3CCc4ccc(OC)cc4C3)cc2C(F)(F)F)C1=O ; *N1Cc2c(cc(CN3CCC(c4ccccc4)C3)cc2C(F)(F)F)C1=O]

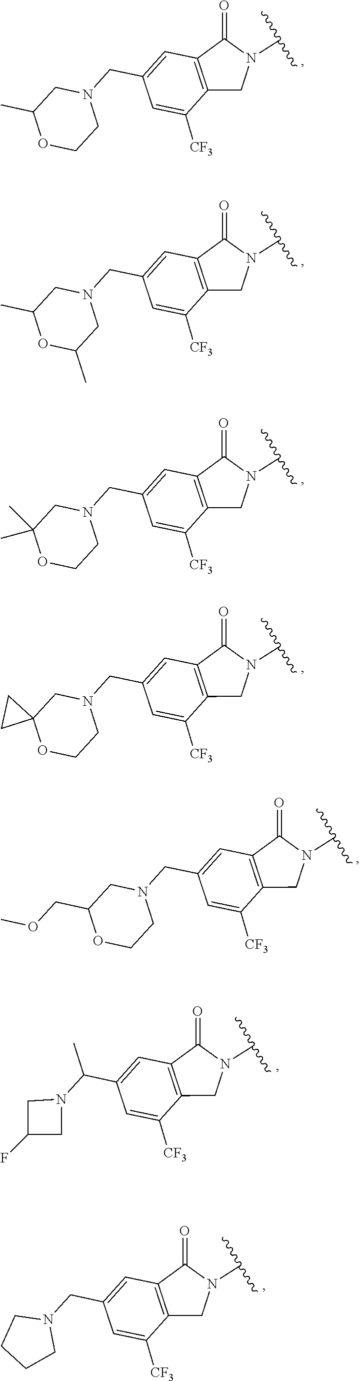

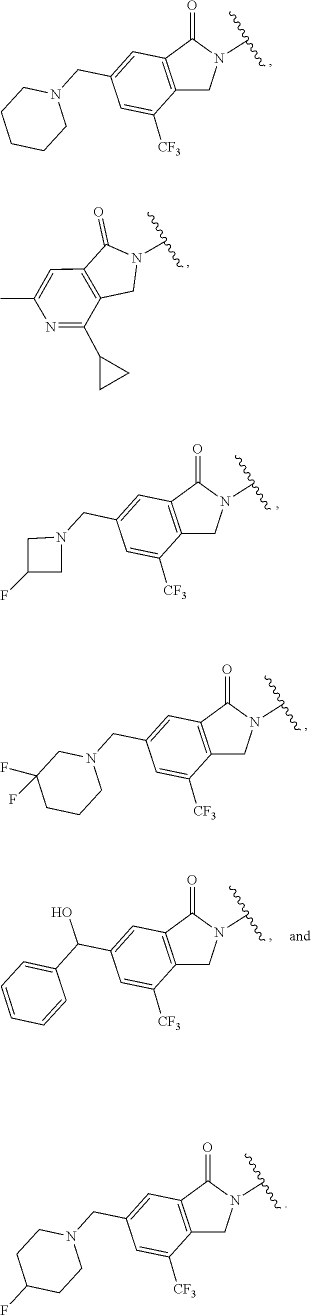 [SMILES: *N1Cc2c(cc(CN3CCCCC3)cc2C(F)(F)F)C1=O ; *N1Cc2c(cc(C)nc2C2CC2)C1=O ; *N1Cc2c(cc(CN3CC(F)C3)cc2C(F)(F)F)C1=O ; *N1Cc2c(cc(CN3CCCC(F)(F)C3)cc2C(F)(F)F)C1=O ; *N1Cc2c(cc(C(O)c3ccccc3)cc2C(F)(F)F)C1=O ; *N1Cc2c(cc(CN3CCC(F)CC3)cc2C(F)(F)F)C1=O]

Embodiment 30. The compound or tautomer thereof, or pharmaceutically acceptable salt of the compound or tautomer, of any one of embodiments 1-29, wherein each K1 is independently selected from the group consisting of:

F, Cl, Br, I, —CN,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl,

—O—C₁-C₈ haloalkyl, and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl.

Embodiment 31. The compound or tautomer thereof, or pharmaceutically acceptable salt of the compound or tautomer, of any one of embodiments 1-29, wherein each K1 is independently

C₃-C₈ cycloalkyl optionally substituted with —C₁-C₈ alkyl, —OH or —O—C₁-C₈ alkyl, or

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO-(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl.

Embodiment 32. The compound or tautomer thereof, or pharmaceutically acceptable salt of the compound or tautomer, of any one of embodiments 1-29, wherein two vicinal K1 groups are taken together with the atoms to which they are attached to form a three- to six-membered carbocyclic or heterocyclic ring, a phenyl ring, or a five- to six-membered heteroaryl ring, wherein the ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

Embodiment 33. The compound or tautomer thereof, or pharmaceutically acceptable salt of the compound or tautomer, of any one of embodiments 1-29, wherein two vicinal K1 groups are taken together with the atoms to which they are attached to form a five-membered carbocyclic or heterocyclic ring, wherein the five-membered carbocyclic or heterocyclic ring are each optionally substituted with one or two substituents selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

Embodiment 34. The compound or tautomer thereof, or pharmaceutically acceptable salt of the compound or tautomer, of any one of embodiments 1-33, wherein m1 is 0.

Embodiment 35. The compound or tautomer thereof, or pharmaceutically acceptable salt of the compound or tautomer, of any one of embodiments 1-33, wherein m1 is 1.

Embodiment 36. The compound or tautomer thereof, or pharmaceutically acceptable salt of the compound or tautomer, of any one of embodiments 1-35, wherein one of R¹⁷ and R¹⁸ is C₁-C₈ alkyl.

Embodiment 37. The compound or tautomer thereof, or pharmaceutically acceptable salt of the compound or tautomer, of any one of embodiments 1-35, wherein R¹⁷ is methyl or R¹⁸ is methyl.

Embodiment 38. The compound or tautomer thereof, or pharmaceutically acceptable salt of the compound or tautomer, of any one of embodiments 1-35, wherein one of R¹⁷ and R¹⁸ is H, F, CF₃ or —CH₂OCH₃.

Embodiment 39. The compound or tautomer thereof, or pharmaceutically acceptable salt of the compound or tautomer, of any one of embodiments 1-35, wherein one of R¹⁷ and R¹⁸ is methyl and the other is H or F.

Embodiment 40. The compound or tautomer thereof, or pharmaceutically acceptable salt of the compound or tautomer, of any one of embodiments 1-35, wherein R¹⁷ and R¹⁸ together with the carbon to which they are attached form a C₃-C₈ cycloalkyl ring or three- to six-membered heterocyclyl ring, wherein the C₃-C₈ cycloalkyl ring or the three- to six-membered heterocyclyl ring is optionally substituted with F, Cl, Br, I, —OH, C₁-C₄ alkyl, or —O—C₁-C₄ alkyl.

Embodiment 41. The compound or tautomer thereof, or pharmaceutically acceptable salt of the compound or tautomer, of any one of embodiments 1-35, wherein R¹⁷ and R¹⁸ together with the carbon to which they are attached form a cyclopropyl or oxetanyl ring, wherein the cyclopropyl or oxetanyl ring is optionally substituted with F, Cl, Br, I, —OH, C₁-C₄ alkyl, or —O—C₁-C₄ alkyl.

Embodiment 42. The compound or tautomer thereof, or pharmaceutically acceptable salt of the compound or tautomer, of any one of embodiments 1-41, wherein Y1 is C(R¹⁹)(R²⁰), wherein R¹⁹ is selected from the group consisting of H, F, Cl, Br, I, and C₁-C₈ alkyl, and R²⁰ is selected from the group consisting of H, F, Cl, Br, I, and C₁-C₈ alkyl.

Embodiment 43. The compound or tautomer thereof, or pharmaceutically acceptable salt of the compound or tautomer, of embodiment 342, wherein Y1 is CH₂.

Embodiment 44. The compound or tautomer thereof, or pharmaceutically acceptable salt of the compound or tautomer, of any one of embodiments 1-41, wherein Y1 is S.

Embodiment 45. The compound or tautomer thereof, or pharmaceutically acceptable salt of the compound or tautomer, of any one of embodiments 1-35, wherein R¹⁹ is taken together with R¹⁸ to form a three- to six-membered cycloalkyl, heterocyclyl, aryl, or heteroaryl ring, each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

Embodiment 46. The compound or tautomer thereof, or pharmaceutically acceptable salt of the compound or tautomer, of any one of embodiments 1-35, wherein R¹⁹ is taken together with R¹⁸ to form a three- to six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

Embodiment 47. The compound or tautomer thereof, or pharmaceutically acceptable salt of the compound or tautomer, of any one of embodiments 1-35, wherein R¹⁹ is taken together with R¹⁸ to form a cyclopropyl, cyclobutyl, cyclopentyl, cyclohexyl, aziridinyl, oxiranyl, thiiranyl, azetidinyl, oxetanyl, thietanyl, pyrrolidinyl, pyrazolidinyl, imidazolidinyl, tetrahydrofuranyl, 1,3-dioxolanyl, tetrahydrothiophenyl, oxathiolanyl, sulfolanyl, piperidinyl, piperazinyl, tetrahydropyranyl, dioxanyl, thianyl, dithianyl, trithianyl, morpholinyl, or thiomorpholinyl ring.

Embodiment 48. The compound or tautomer thereof, or pharmaceutically acceptable salt of the compound or tautomer, of any one of embodiments 1-35, wherein Y1 is C(R¹⁹)(R²⁰), and the absolute configuration of the carbon atom to which R¹⁷ and R¹⁸ are attached is (R).

Embodiment 49. The compound or tautomer thereof, or pharmaceutically acceptable salt of the compound or tautomer, of any one of embodiments 1-35, wherein Y1 is C(R¹⁹)(R²⁰), and the absolute configuration of the carbon atom to which R¹⁷ and R¹⁸ are attached is (S).

Embodiment 50. The compound or tautomer thereof, or pharmaceutically acceptable salt of the compound or tautomer, of any one of embodiments 1-35, wherein Y1 is S, and the absolute configuration of the carbon atom to which R¹⁷ and R¹⁸ are attached is (S).

Embodiment 51. The compound or tautomer thereof, or pharmaceutically acceptable salt of the compound or tautomer, of any one of embodiments 1-35, wherein Y1 is S, and the absolute configuration of the carbon atom to which R¹⁷ and R¹⁸ are attached is (R).

Embodiment 52. The compound or tautomer thereof, or pharmaceutically acceptable salt of the compound or tautomer, of embodiment 1, wherein [symbol] is a double bond, Y1 is C(R¹⁹), and Y2 is C(R¹⁸); and R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl.

Embodiment 53. The compound or tautomer thereof, or pharmaceutically acceptable salt of the compound or tautomer, of any one of embodiments 1-52, wherein Ring B1 is a five-membered heteroaryl ring containing at least one N, O, or S ring atom, wherein Ring B1 is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 54. The compound or tautomer thereof, or pharmaceutically acceptable salt of the compound or tautomer, of any one of embodiments 1-52, wherein Ring B1 is a five-membered heteroaryl ring containing at least one N, O, or S ring atom, wherein Ring B1 is optionally substituted with one, two, or three substituents independently selected from the group consisting of F, Cl, Br, or I.

Embodiment 55. The compound or tautomer thereof, or pharmaceutically acceptable salt of the compound or tautomer, of any one of embodiments 1-52, wherein Ring B1 is a five-membered heteroaryl ring containing at least one N, O, or S ring atom, wherein Ring B1 is optionally substituted with one, two, or three substituents independently selected from the group consisting of methyl, ethyl, cyclopropyl, and —CH₂OH.

Embodiment 56. The compound or tautomer thereof, or pharmaceutically acceptable salt of the compound or tautomer, of any one of embodiments 1-52, wherein Ring B1 is selected from the group consisting of pyrrole, imidazole, 1,2,4-triazole, 1,2,3-triazole, pyrazole, tetrazole, oxadiazole, oxazole, and isoxazole, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 57. The compound or tautomer thereof, or pharmaceutically acceptable salt of the compound or tautomer, of any one of embodiments 1-52, wherein Ring B1 is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 58. The compound or tautomer thereof, or pharmaceutically acceptable salt of the compound or tautomer, of any one of embodiments 1-52, wherein Ring B1 is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with methyl, ethyl, cyclopropyl, or —CH₂OH.

Embodiment 59. The compound or tautomer thereof, or pharmaceutically acceptable salt of the compound or tautomer, of any one of embodiments 1-52, wherein Ring B1 is selected from the group consisting of

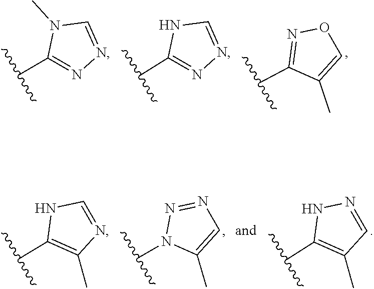

Embodiment 60. The compound or tautomer thereof, or pharmaceutically acceptable salt of the compound or tautomer, of any one of embodiments 1-52, wherein Ring B1 is selected from the group consisting of

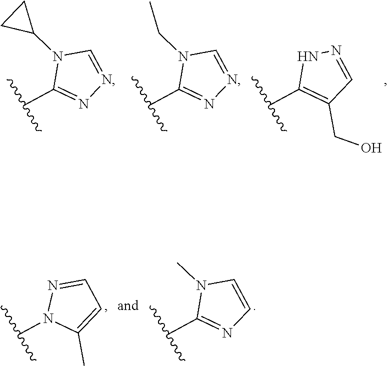

Embodiment 61. The compound or tautomer thereof, or pharmaceutically acceptable salt of the compound or tautomer, of any one of embodiments 1-52, wherein Ring B1 is selected from the group consisting of

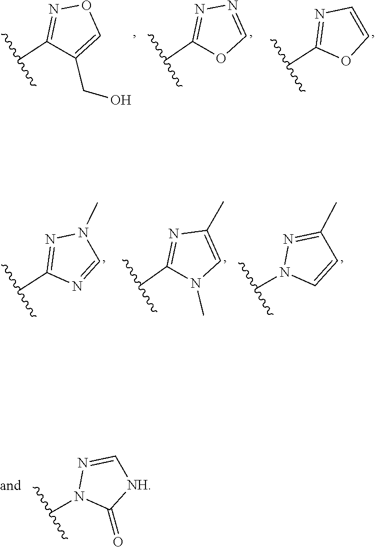

Embodiment 62. The compound or tautomer thereof, or pharmaceutically acceptable salt of the compound or tautomer, of any one of embodiments 1-52, wherein Ring B1 is selected from the group consisting of

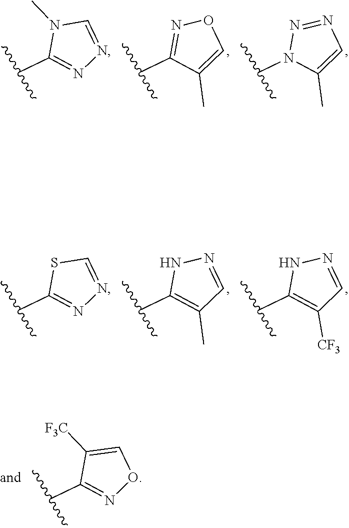

Embodiment 63. The compound or tautomer thereof, or pharmaceutically acceptable salt of the compound or tautomer, of any one of embodiments 1-52, wherein Ring B1 is 4-methyl-4H-1,2,4-triazol-3-yl.

Embodiment 64. The compound or tautomer thereof, or pharmaceutically acceptable salt of the compound or tautomer, of embodiment 1, selected from the group consisting of Compounds 86, 162-169, 171-180, 255a-283b, 289-301, and 304a-304b of Table 1, including “a” and “b” variants of the compounds, tautomers thereof, and pharmaceutically acceptable salts of the compounds or tautomers.

Embodiment 65. The compound or tautomer thereof, or pharmaceutically acceptable salt of the compound or tautomer, of embodiment 1, which is compound 255a, a tautomer thereof, or pharmaceutically acceptable salt of the compound or tautomer.

Embodiment 66. The compound or tautomer thereof, or pharmaceutically acceptable salt of the compound or tautomer, of embodiment 1, which is compound 282, a tautomer thereof, or pharmaceutically acceptable salt of the compound or tautomer.

Embodiment 67. The compound or tautomer thereof, or pharmaceutically acceptable salt of the compound or tautomer, of embodiment 1, which is a compound selected from the group consisting of

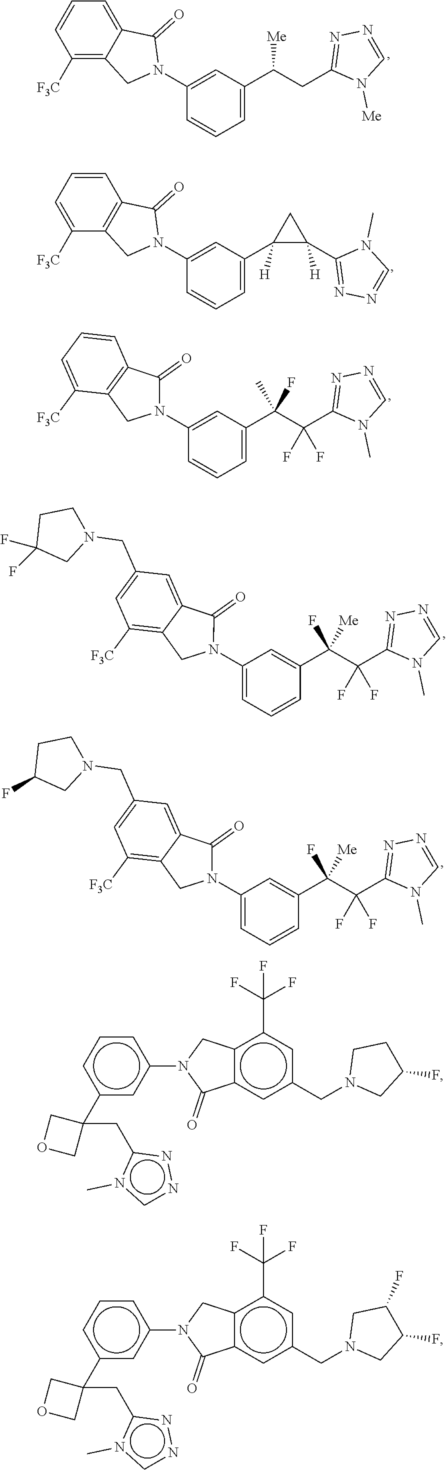

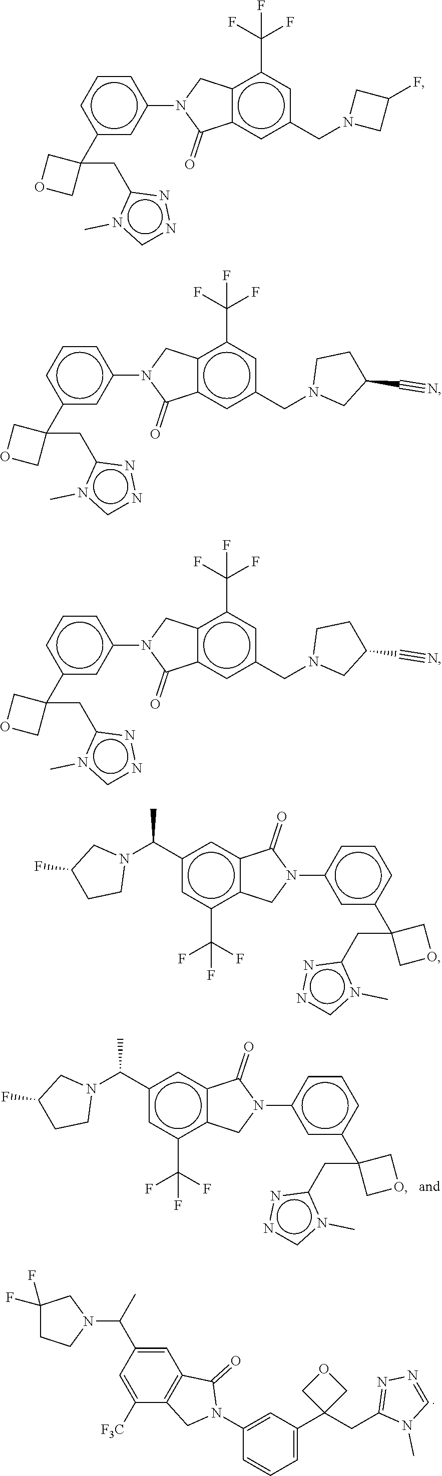

Embodiment 68. A compound of Formula (II-A):

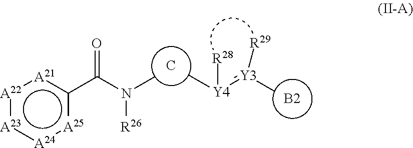

or a tautomer thereof, or a pharmaceutically acceptable salt of any of the foregoing, wherein

A²¹ is CR²¹ or N, or is absent,

A²² is CR²² or N,

A²³ is CR²³ or N,

A²⁴ is CR²⁴ or N, and

A²⁵ is CR²⁵ or N,

wherein no more than two of A²¹, A²², A²³, A²⁴, and A²⁵ are N;

R²¹, R²², R²³, and R²⁴ are independently selected from R^{x};

each R^{x} is independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{p}R^{q} where R^{p} and R^{q} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{q} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{p} and R^{q} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a —C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{r}R^{s}, —O—(C₁-C₄ alkylene)-NR^{r}R^{s}, —C(═O)NR^{r}R^{s}, —(C₁-C₄ alkylene)-C(═O)NR^{r}R^{s}, or —O—(C₁-C₄ alkylene)-C(═O)NR^{r}R^{s}, wherein R^{r} and R^{s} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{r} and R^{s} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{t}R^{u} wherein R and R^{u} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{t} and R^{u} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R²⁵ is independently selected from R^{x}, and R²⁶ is H; or

A₂₅ is CR²⁵, and R²⁵, R²⁶ and the intervening atoms are taken together to form a five-membered lactam ring, such that the fragment

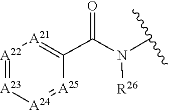

is

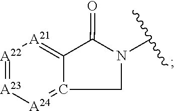

or

(R²¹ and R²²) or (R²² and R²³) or (R²³ and R²⁴) or (R²⁴ and R²⁵), together with the atoms to which they are attached, are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl);

Ring C,

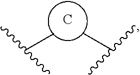

is selected from the group consisting of:

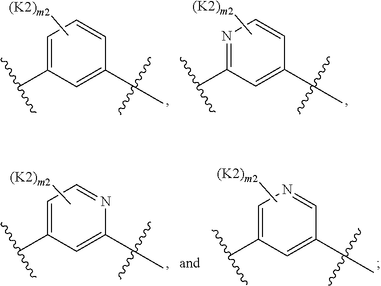

each K2 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —C₁-C₈ alkyl, —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO-(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K2 groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m2 is 0, 1, or 2;

[symbol] is a single bond or a double bond,

wherein when [symbol] is a single bond, Y3 is C(R³⁰) and Y4 is C(R²⁷); and

when [symbol] is a double bond, Y3 is C and Y4 is C;

R²⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl;

R²⁸ and R²⁹, as indicated by the dashed curve [symbol] are taken together with the atoms to which they are attached to form a three- to six-membered cycloalkyl, heterocyclyl, aryl, or heteroaryl ring, wherein the three- to six-membered cycloalkyl, heterocyclyl, aryl, or heteroaryl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl,

R³⁰ is selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; and

Ring B2,

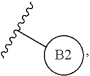

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 69. The compound of embodiment 68, wherein the compound of Formula (II-A) is a compound of Formula (II):

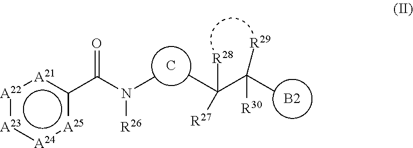

or a tautomer thereof, or a pharmaceutically acceptable salt of any of the foregoing, wherein

A²¹ is CR²¹ or N, or is absent,

A²² is CR²² or N,

A²³ is CR²³ or N,

A²⁴ is CR²⁴ or N, and

A²⁵ is CR²⁵ or N,

wherein no more than two of A²¹, A²², A²³, A²⁴, and A²⁵ are N;

R²¹, R²², R²³, and R²⁴ are independently selected from R^{x};

each R^{x} is independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{p}R^{q} where R^{p} and R^{q} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{q} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{p} and R^{q} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, CN, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{r}R^{s}, —O—(C₁-C₄ alkylene)-NR^{r}R^{s}, —C(═O)NR^{r}R^{s}, —(C₁-C₄ alkylene)-C(═O)NR^{r}R^{s}, or —O—(C₁-C₄ alkylene)-C(═O)NR^{r}R^{s}, wherein R^{r} and R^{s} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{r} and R^{s} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{t}R^{u} wherein R^{t} and R^{u} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{t} and R^{u} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R²⁵ is independently selected from R^{x}, and R²⁶ is H; or

A₂₅ is CR²⁵, and R²⁵, R²⁶ and the intervening atoms are taken together to form a five-membered lactam ring, such that the fragment

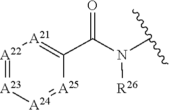

is

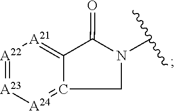

or

(R²¹ and R²²) or (R²² and R²³) or (R²³ and R²⁴) or (R²⁴ and R²⁵), together with the atoms to which they are attached, are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl);

Ring C,

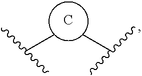

is selected from the group consisting of:

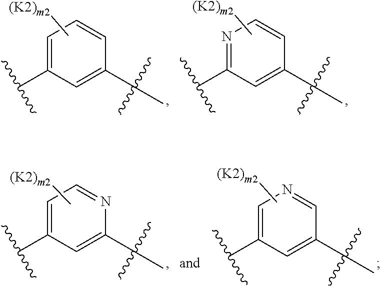

each K2 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, and four- to eight-membered heterocyclyl optionally substituted with —OH or C₁-C₄ alkyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K2 groups are taken together with the atoms to which they are attached to form a three- to six-membered carbocyclic or heterocyclic ring, a phenyl ring, or a five- to six-membered heteroaryl ring, wherein the ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m2 is 0, 1, or 2;

R²⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl;

R²⁸ and R²⁹, as indicated by the dashed curve [symbol] are taken together with the atoms to which they are attached to form a three- to six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl,

R³⁰ is selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; and

Ring B2,

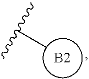

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 70. The compound or tautomer thereof, or pharmaceutically acceptable salt of the compound or tautomer, of embodiment 68 or embodiment 69, wherein

A²¹ is CR²¹ or N, or is absent,

A²² is CR²² or N,

A²³ is CR²³ or N,

A²⁴ is CR²⁴ or N, and

A²⁵ is CR²⁵ or N,

wherein no more than two of A²¹, A²², A²³, A²⁴, and A²⁵ are N;

R²¹, R²², R²³, and R²⁴ are independently selected from R^{x};
each R^{x} is independently selected from the group consisting of:

H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{p}R^{q} where R^{p} and R^{q} are independently H, C₁-C₈ alkyl, C₁-C₈ haloalkyl, or C₃-C₈ cycloalkyl, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{q} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{p} and R^{q} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, or I, —(C₁-C₄ alkylene)-NR^{r}R^{s}, —O—(C₁-C₄ alkylene)-NR^{r}R^{s}, —C(═O)NR^{r}R^{s}, —(C₁-C₄ alkylene)-C(═O)NR^{r}R^{s}, or —O—(C₁-C₄ alkylene)-C(═O)NR^{r}R^{s}, wherein R^{r} and R^{s} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{r} and R^{s} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{t}R^{u} wherein R^{t} and R^{u} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{t} and R^{u} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R²⁵ is independently selected from R^{x}, and R²⁶ is H; or

A₂₅ is CR²⁵, and R²⁵, R²⁶ and the intervening atoms are taken together to form a five-membered lactam ring, such that the fragment

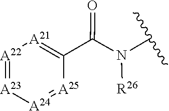

is

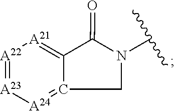

or

(R²¹ and R²²) or (R²² and R²³) or (R²³ and R²⁴) or (R²⁴ and R²⁵), together with the atoms to which they are attached, are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl);

Ring C,

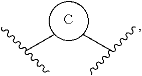

is selected from the group consisting of:

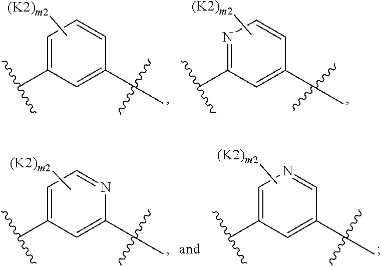

each K2 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl,

—O—C₁-C₈ haloalkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K2 groups are taken together with the atoms to which they are attached to form a three- to six-membered carbocyclic or heterocyclic ring, a phenyl ring, or a five- to six-membered heteroaryl ring, wherein the ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl; and

m2 is 0, 1, or 2;

R²⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl;

R²⁸ and R²⁹, as indicated by the dashed curve [symbol] , are taken together with the atoms to which they are attached to form a three- to six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl;

R³⁰ is selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; and

Ring B2,

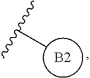

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 71. The compound or tautomer thereof, or pharmaceutically acceptable salt of the compound or tautomer, of any one of embodiments 68-70, wherein A²¹ is CR²¹ or N.

Embodiment 72. The compound or tautomer thereof, or pharmaceutically acceptable salt of the compound or tautomer, of any one of embodiments 68-71, wherein Ring C,

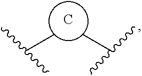

is

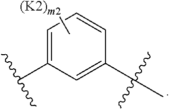

Embodiment 73. The compound or tautomer thereof, or pharmaceutically acceptable salt of the compound or tautomer, of any one of embodiments 68-72, wherein R²¹, R²², R²³, and R²⁴ are independently selected from the group consisting of

F, Cl, Br, I, —CN, C₁-C₈ alkyl, —O—C₁-C₈ alkyl, —C₁-C₈ alkyl-OH, C₁-C₈ haloalkyl, C₃-C₆ cycloalkyl, —S(═O)₂—C₁-C₈ alkyl, —(C₁-C₄ alkylene)-NR^{r}R^{s}, and —C(═O)NR^{r}R^{s},

wherein R^{r} and R^{s} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

Embodiment 74. The compound or tautomer thereof, or pharmaceutically acceptable salt of the compound or tautomer, of any one of embodiments 68-73, wherein R²¹ and R²², together with the atoms to which they are attached are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(Cᵣ C₄ alkyl), or —N(C₁-C₄ alkyl)(C₁-C₄ alkyl).

Embodiment 75. The compound or tautomer thereof, or pharmaceutically acceptable salt of the compound or tautomer, of any one of embodiments 68-72, wherein R²² and R²³, together with the atoms to which they are attached are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(Cᵣ C₄ alkyl), or —N(C₁-C₄ alkyl)(C₁-C₄ alkyl).

Embodiment 76. The compound or tautomer thereof, or pharmaceutically acceptable salt of the compound or tautomer, of any one of embodiments 68-72, wherein R²³ and R²⁴, together with the atoms to which they are attached are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(Cᵣ C₄ alkyl), or —N(C₁-C₄ alkyl)(C₁-C₄ alkyl).

Embodiment 77. The compound or tautomer thereof, or pharmaceutically acceptable salt of the compound or tautomer, of any one of embodiments 68-72, wherein R²⁴ and R²⁵, together with the atoms to which they are attached are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(Cᵣ C₄ alkyl), or —N(C₁-C₄ alkyl)(C₁-C₄ alkyl).

Embodiment 78. The compound or tautomer thereof, or pharmaceutically acceptable salt of the compound or tautomer, of any one of embodiments 68-72, wherein

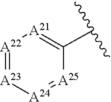

is selected from the group consisting of

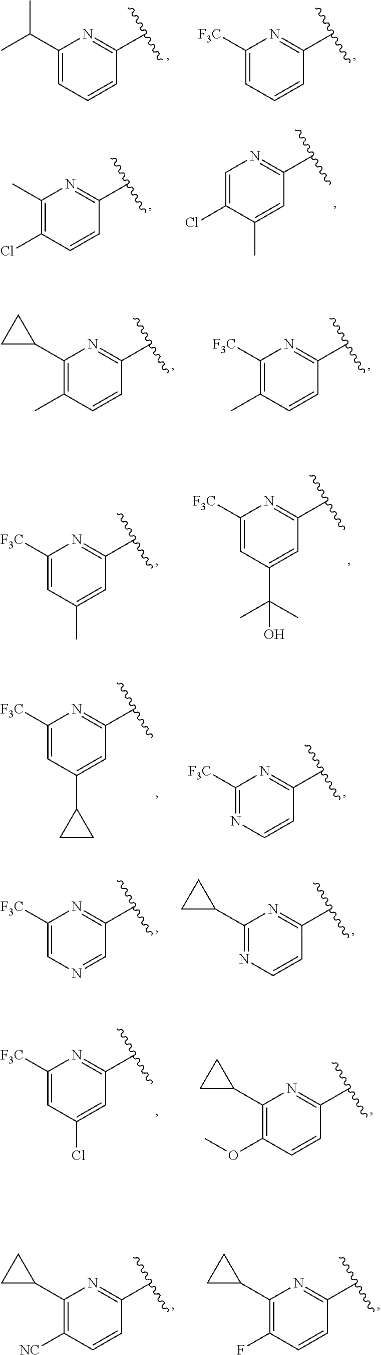

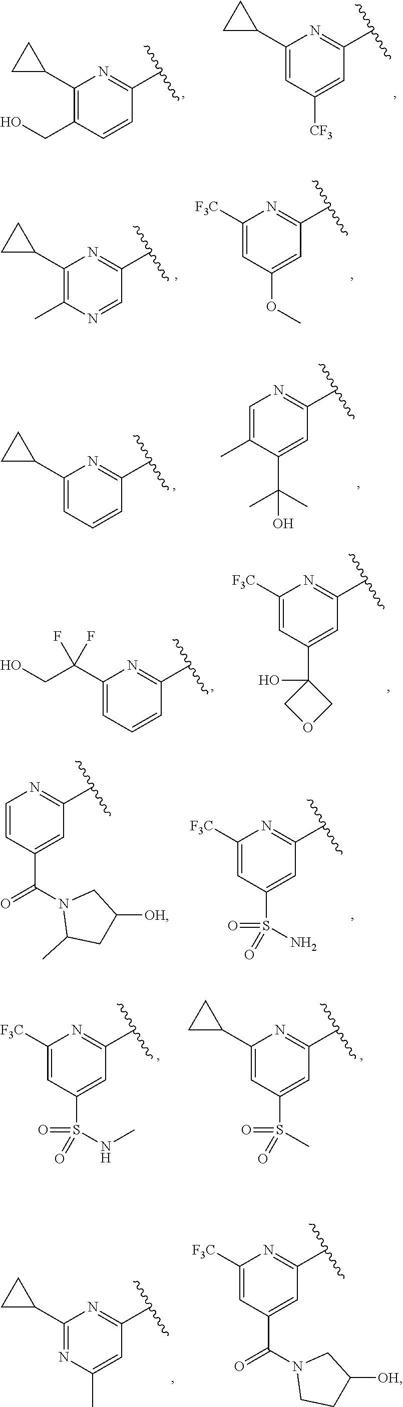

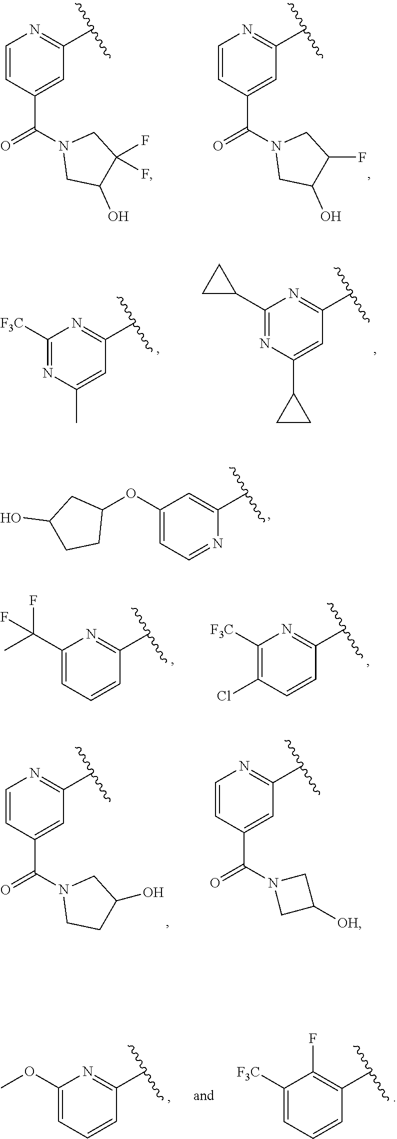

Embodiment 79. The compound or tautomer thereof, or pharmaceutically acceptable salt of the compound or tautomer, of any one of embodiments 68-72, wherein

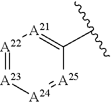

is selected from the group consisting of

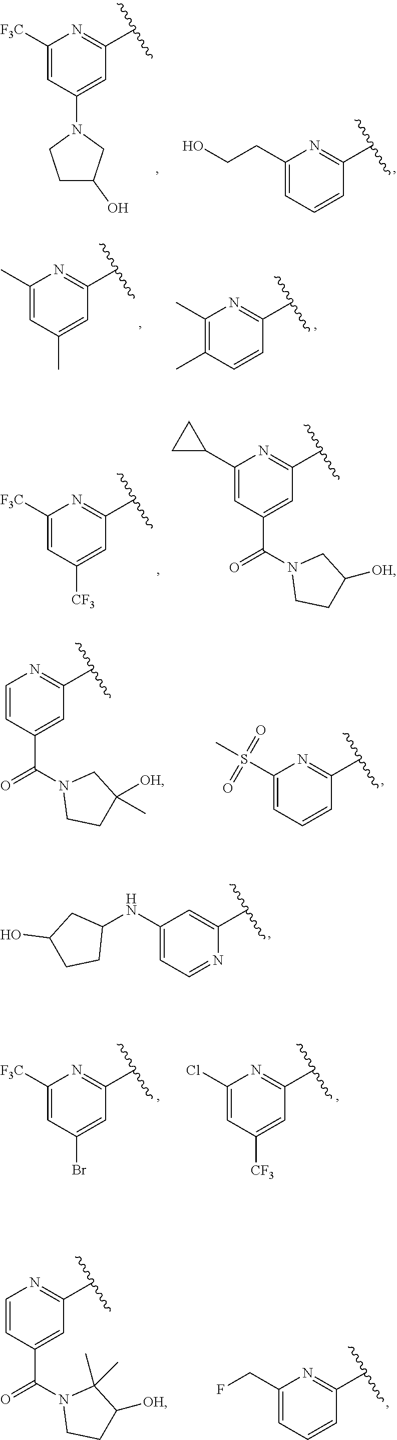

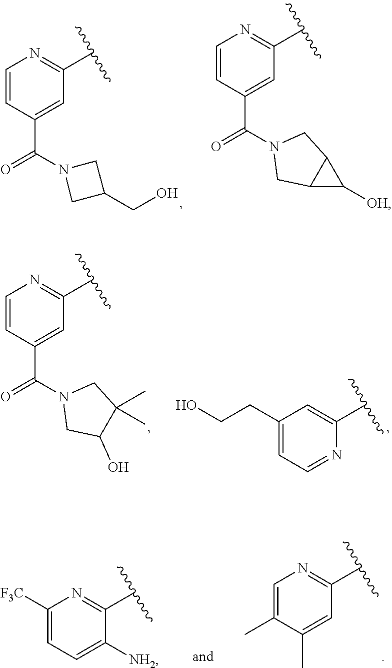

Embodiment 80. The compound or tautomer thereof, or pharmaceutically acceptable salt of the compound or tautomer, of any one of embodiments 68-72, wherein

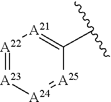

is selected from the group consisting of

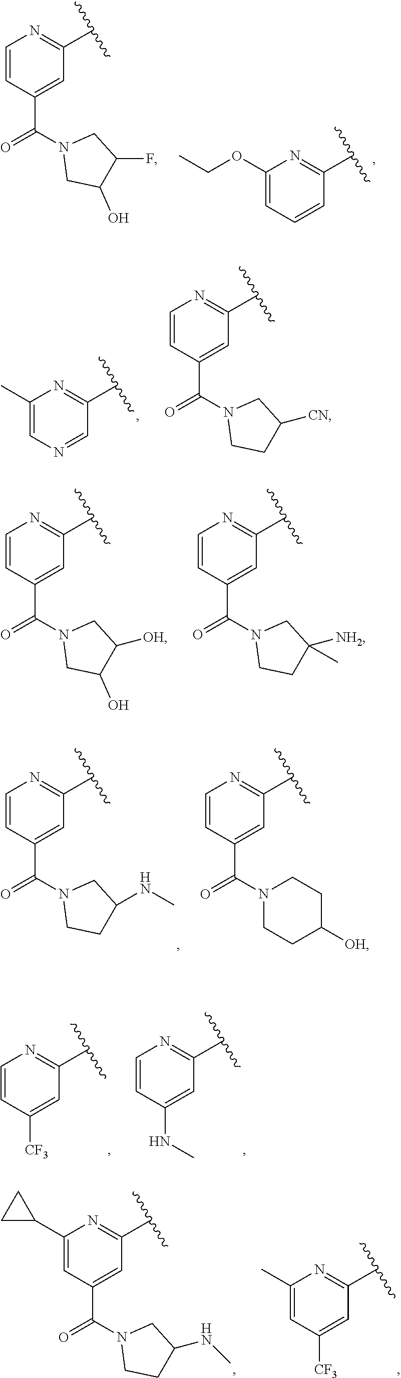

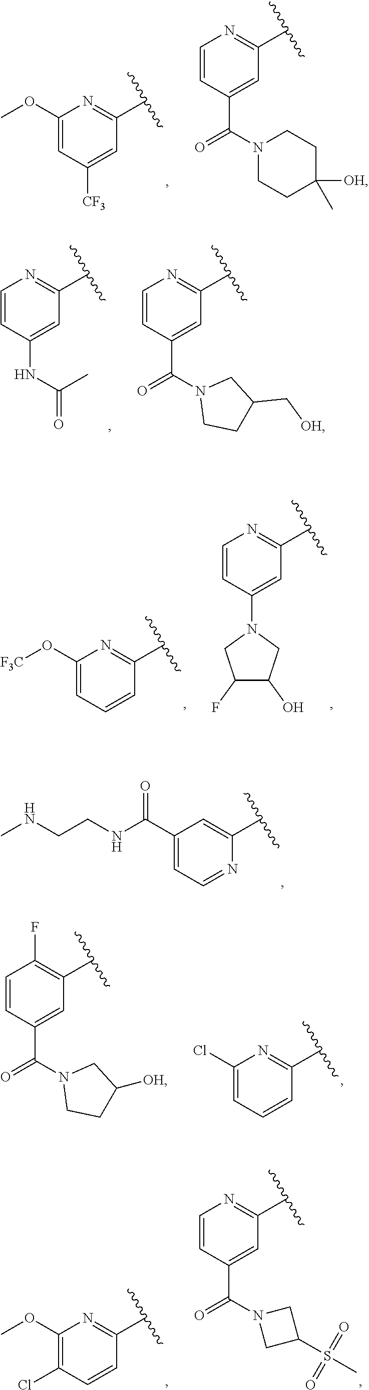

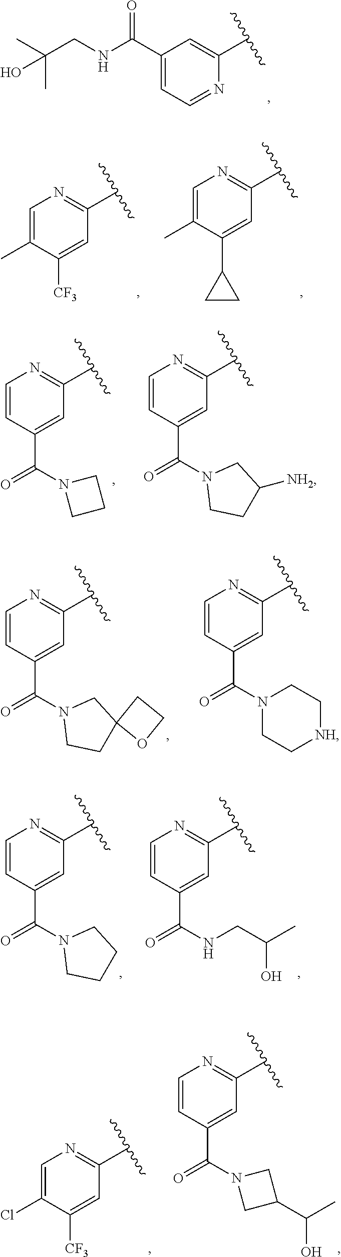

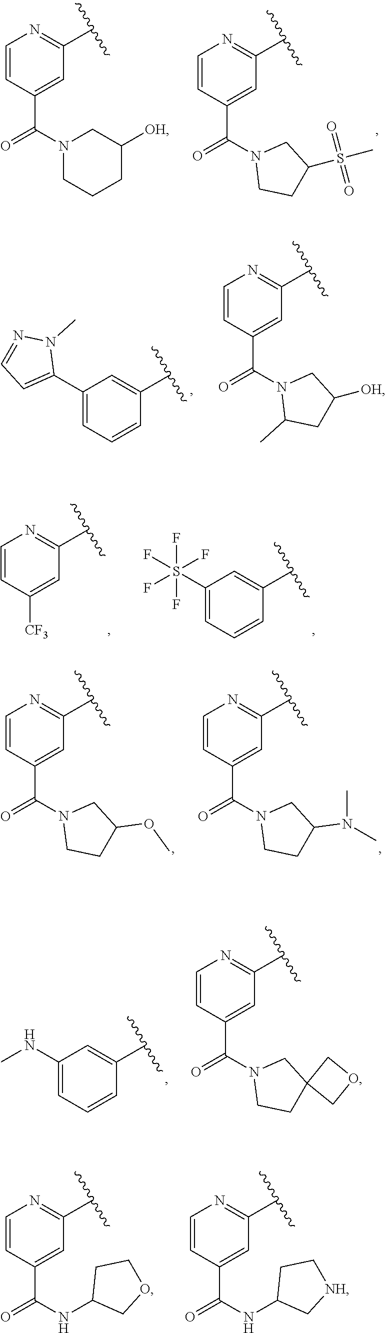

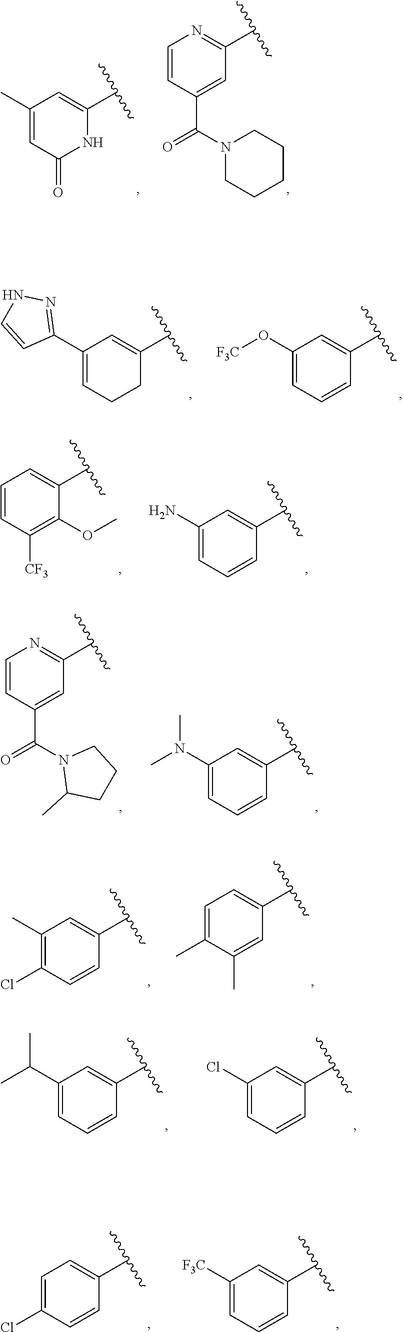

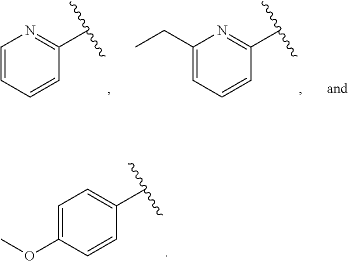

Embodiment 81. The compound or tautomer thereof, or pharmaceutically acceptable salt of the compound or tautomer, of any one of embodiments 68-72, wherein

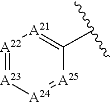

is selected from the group consisting of

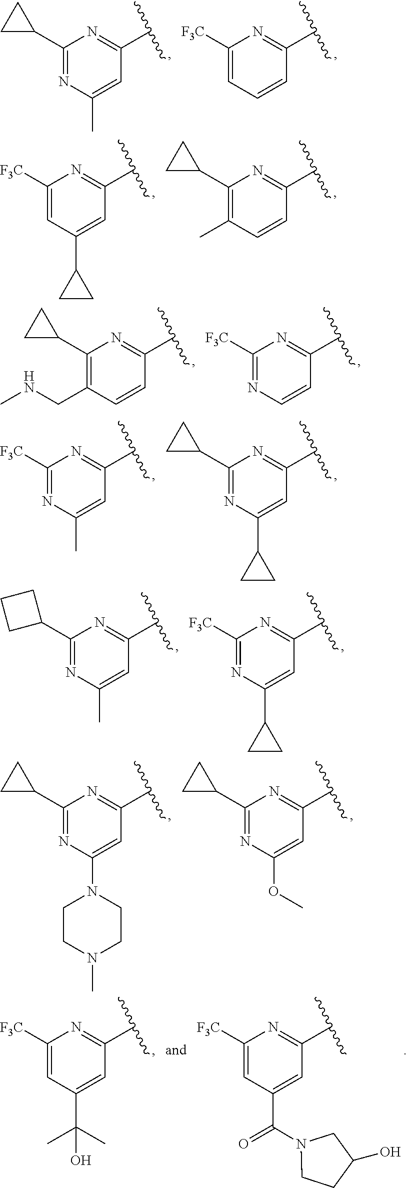

Embodiment 82. The compound or tautomer thereof, or pharmaceutically acceptable salt of the compound or tautomer, of any one of embodiments 68-72, wherein

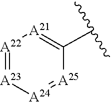

is selected from the group consisting of

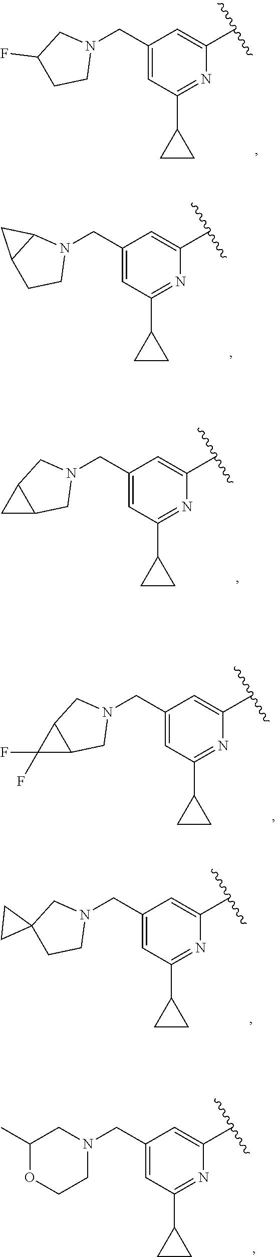 [SMILES: *c1cc(CN2CCC(F)C2)cc(C2CC2)n1 ; *c1cc(CN2CCC3CC32)cc(C2CC2)n1 ; *c1cc(CN2CC3CC3C2)cc(C2CC2)n1 ; *c1cc(CN2CC3C(C2)C3(F)F)cc(C2CC2)n1 ; *c1cc(CN2CCC3(CC3)C2)cc(C2CC2)n1 ; *c1cc(CN2CCOC(C)C2)cc(C2CC2)n1]

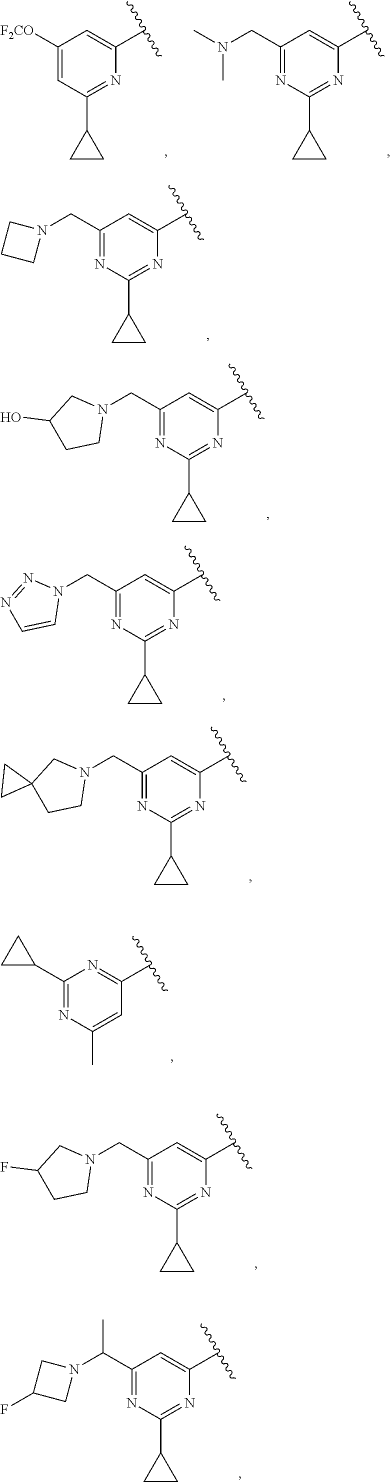

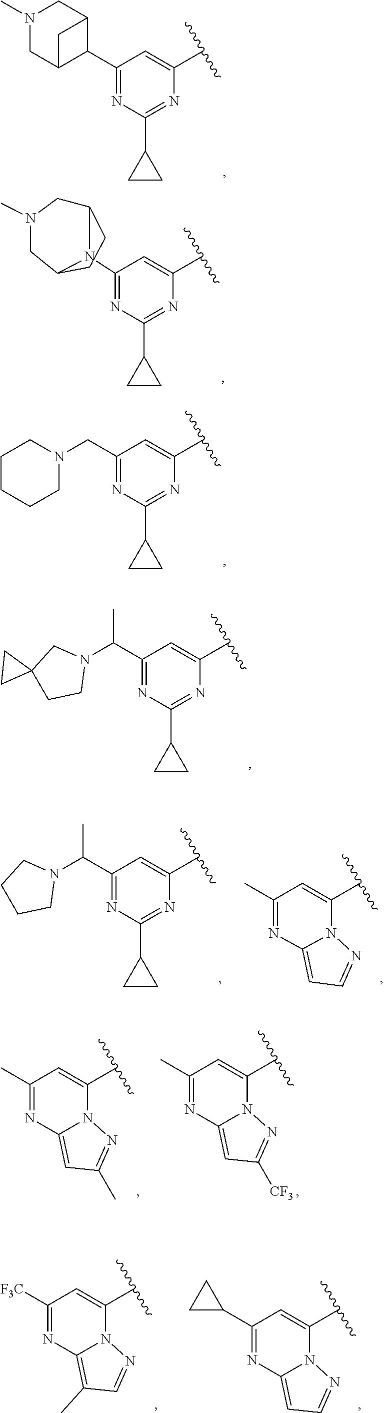

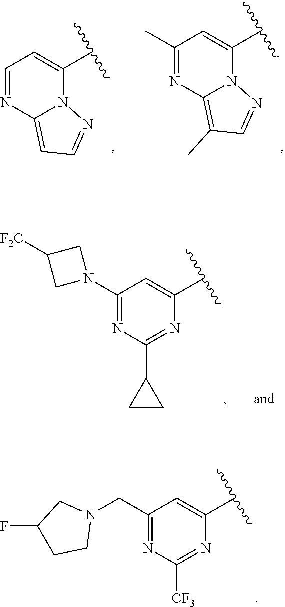

Embodiment 83. The compound or tautomer thereof, or pharmaceutically acceptable salt of the compound or tautomer, of any one of embodiments 68-82, wherein each K2 is independently selected from the group consisting of:

F, Cl, Br, I, —CN,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl,

—O—C₁-C₈ haloalkyl, and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl.

Embodiment 84. The compound or tautomer thereof, or pharmaceutically acceptable salt of the compound or tautomer, of any one of embodiments 68-82, wherein each K2 is independently

C₃-C₈ cycloalkyl optionally substituted with —C₁-C₈ alkyl, —OH or —O—C₁-C₈ alkyl, or

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO-(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, Ci-C's hydroxyalkyl, —CN, or —O—C₁-C₈ alkyl.

Embodiment 85. The compound or tautomer thereof, or pharmaceutically acceptable salt of the compound or tautomer, of any one of embodiments 68-82, wherein two vicinal K2 groups are taken together with the atoms to which they are attached to form a three- to six-membered carbocyclic or heterocyclic ring, a phenyl ring, or a five- to six-membered heteroaryl ring, wherein the ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

Embodiment 86. The compound or tautomer thereof, or pharmaceutically acceptable salt of the compound or tautomer, of any one of embodiments 68-82, wherein two vicinal K2 groups are taken together with the atoms to which they are attached to form a five-membered carbocyclic or heterocyclic ring, wherein the five-membered carbocyclic or heterocyclic ring are each optionally substituted with one or two substituents selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

Embodiment 87. The compound or tautomer thereof, or pharmaceutically acceptable salt of the compound or tautomer, of any one of embodiments 68-84, wherein m2 is 0.

Embodiment 88. The compound or tautomer thereof, or pharmaceutically acceptable salt of the compound or tautomer, of any one of embodiments 68-84, wherein m2 is 1.

Embodiment 89. The compound or tautomer thereof, or pharmaceutically acceptable salt of the compound or tautomer, of any one of embodiments 68-88, wherein R²⁶ is H.

Embodiment 90. The compound or tautomer thereof, or pharmaceutically acceptable salt of the compound or tautomer, of any one of embodiments 68-76 or 83-88, wherein A₂₅ is CR²⁵, and R²⁵, R²⁶ and the intervening atoms are taken together to form a five-membered lactam ring, such that the fragment

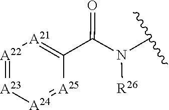

is

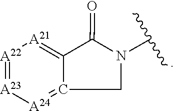

Embodiment 91. The compound or tautomer thereof, or pharmaceutically acceptable salt of the compound or tautomer, of embodiment 90, wherein at least one of R²¹, R²², R²³, and R²⁴ is selected from the group consisting of: a three- to eight-membered heterocyclic ring, a five-to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 92. The compound or tautomer thereof, or pharmaceutically acceptable salt of the compound or tautomer, of embodiment 90, wherein R²² is a —(C₁-C₄ alkylene)-(four- to eight-membered heterocyclic ring), wherein the heterocyclic ring is optionally substituted with one, two, or three substituents independently selected from the group consisting of —OH, oxo, C₃-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 93. The compound or tautomer thereof, or pharmaceutically acceptable salt of the compound or tautomer, of embodiment 90, wherein R²² is a —(C₁-C₂ alkylene)-(four-membered heterocyclic ring), a —(C₁-C₂ alkylene)-(five-membered heterocyclic ring), or a —(C₁-C₂ alkylene)-(six-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 94. The compound or tautomer thereof, or pharmaceutically acceptable salt of the compound or tautomer, of embodiment 90, wherein R²² is a —CH₂-(four- to eight-membered heterocyclic ring) or a —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₃-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 95. The compound or tautomer thereof, or pharmaceutically acceptable salt of the compound or tautomer, of embodiment 90, wherein R²² is —CH₂-(pyrrolidinyl) or —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 96. The compound or tautomer thereof, or pharmaceutically acceptable salt of the compound or tautomer, of embodiment 90, wherein R²² is —CH₂-(azetidinyl) or —CH(CH₃)-azetidinyl, wherein the azetidinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 97. The compound or tautomer thereof, or pharmaceutically acceptable salt of the compound or tautomer, of any one of embodiments 90-96, wherein R²⁴ is selected from the group consisting of H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl.

Embodiment 98. The compound or tautomer thereof, or pharmaceutically acceptable salt of the compound or tautomer, of any one of embodiments 90-96, wherein R²⁴ is CF₃.

Embodiment 99. The compound or tautomer thereof, or pharmaceutically acceptable salt of the compound or tautomer, of embodiment 90, wherein

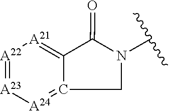

is selected from the group consisting of

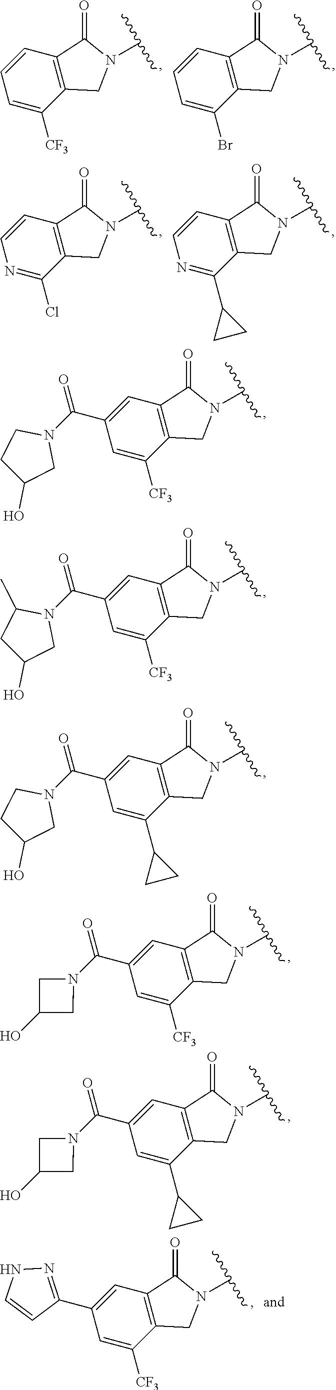

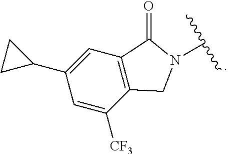 [SMILES: *N1Cc2c(cc(C3CC3)cc2C(F)(F)F)C1=O]

Embodiment 100. The compound or tautomer thereof, or pharmaceutically acceptable salt of the compound or tautomer, of embodiment 90, wherein

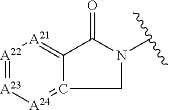

is selected from the group consisting of

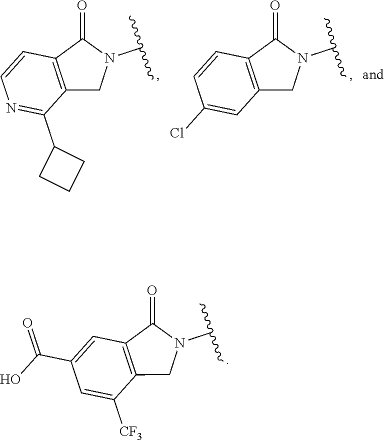 [SMILES: *N1Cc2c(ccnc2C2CCC2)C1=O ; *N1Cc2cc(Cl)ccc2C1=O ; *N1Cc2c(cc(C(=O)O)cc2C(F)(F)F)C1=O]

Embodiment 101. The compound or tautomer thereof, or pharmaceutically acceptable salt of the compound or tautomer, of embodiment 90, wherein

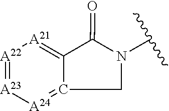

is selected from the group consisting of

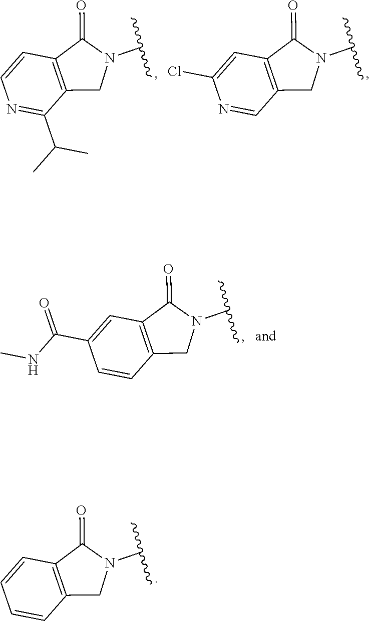

Embodiment 102. The compound or tautomer thereof, or pharmaceutically acceptable salt of the compound or tautomer, of any one of embodiments 68-101, wherein R²⁷ is methyl.

Embodiment 103. The compound or tautomer thereof, or pharmaceutically acceptable salt of the compound or tautomer, of any one of embodiments 68-101, wherein R²⁷ is H, F, CF₃ or —CH₂OCH₃.

Embodiment 104. The compound or tautomer thereof, or pharmaceutically acceptable salt of the compound or tautomer, of any one of embodiments 68-101, wherein R²⁸ and R²⁹ are taken together with the atoms to which they are attached to form a five- or six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

Embodiment 105. The compound or tautomer thereof, or pharmaceutically acceptable salt of the compound or tautomer, of any one of embodiments 68-103, wherein R²⁸ and R²⁹ are taken together to form a cyclopropyl, cyclobutyl, cyclopentyl, cyclohexyl, aziridinyl, oxiranyl, thiiranyl, azetidinyl, oxetanyl, thietanyl, pyrrolidinyl, pyrazolidinyl, imidazolidinyl, tetrahydrofuranyl, 1,3-dioxolanyl, tetrahydrothiophenyl, oxathiolanyl, sulfolanyl, piperidinyl, piperazinyl, tetrahydropyranyl, dioxanyl, thianyl, dithianyl, trithianyl, morpholinyl, or thiomorpholinyl ring.

Embodiment 106. The compound or tautomer thereof, or pharmaceutically acceptable salt of the compound or tautomer, of any one of embodiments 68-105, wherein R³⁰ is selected from the group consisting of H, F, Cl, Br, I, and C₁-C₈ alkyl.

Embodiment 107. The compound or tautomer thereof, or pharmaceutically acceptable salt of the compound or tautomer, of embodiment 106, wherein R³⁰ is H.

Embodiment 108. The compound or tautomer thereof, or pharmaceutically acceptable salt of the compound or tautomer, of any one of embodiments 68-107, wherein the absolute configuration of the carbon atom to which R²⁷ and R²⁸ are attached is (R).

Embodiment 109. The compound or tautomer thereof, or pharmaceutically acceptable salt of the compound or tautomer, of any one of embodiments 68-107, wherein the absolute configuration of the carbon atom to which R²⁷ and R²⁸ are attached is (S).

Embodiment 110. The compound or tautomer thereof, or pharmaceutically acceptable salt of the compound or tautomer, of embodiment 68, wherein [symbol] is a double bond, Y3 is C and Y4 is C.

Embodiment 111. The compound or tautomer thereof, or pharmaceutically acceptable salt of the compound or tautomer, of any one of embodiments 68-109, wherein Ring B2 is a five-membered heteroaryl ring containing at least one N, O, or S ring atom, wherein Ring B2 is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 112. The compound or tautomer thereof, or pharmaceutically acceptable salt of the compound or tautomer, of any one of embodiments 68-109, wherein Ring B2 is a five-membered heteroaryl ring containing at least one N, O, or S ring atom, wherein Ring B2 is optionally substituted with one, two, or three substituents independently selected from the group consisting of F, Cl, Br, and I.

Embodiment 113. The compound or tautomer thereof, or pharmaceutically acceptable salt of the compound or tautomer, of any one of embodiments 68-109, wherein Ring B2 is a five-membered heteroaryl ring containing at least one N, O, or S ring atom, wherein Ring B2 is optionally substituted with one, two, or three substituents independently selected from the group consisting of methyl, ethyl, cyclopropyl, and —CH₂OH.

Embodiment 114. The compound or tautomer thereof, or pharmaceutically acceptable salt of the compound or tautomer, of any one of embodiments 68-109, wherein Ring B2 is selected from the group consisting of pyrrole, imidazole, 1,2,4-triazole, 1,2,3-triazole, pyrazole, tetrazole, oxadiazole, oxazole, and isoxazole, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 115. The compound or tautomer thereof, or pharmaceutically acceptable salt of the compound or tautomer, of any one of embodiments 68-109, wherein Ring B2 is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 116. The compound or tautomer thereof, or pharmaceutically acceptable salt of the compound or tautomer, of any one of embodiments 68-109, wherein Ring B2 is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with methyl, ethyl, cyclopropyl, or —CH₂OH.

Embodiment 117. The compound or tautomer thereof, or pharmaceutically acceptable salt of the compound or tautomer, of any one of embodiments 68-109, wherein Ring B2 is selected from the group consisting of

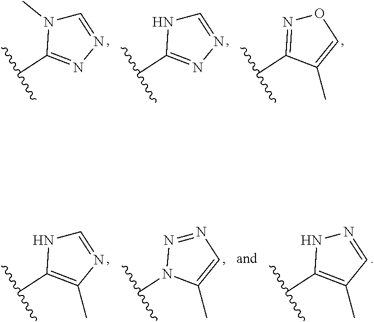

Embodiment 118. The compound or tautomer thereof, or pharmaceutically acceptable salt of the compound or tautomer, of any one of embodiments 68-109, wherein Ring B2 is selected from the group consisting of

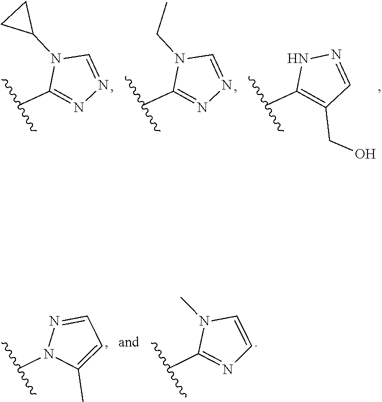

Embodiment 119. The compound or tautomer thereof, or pharmaceutically acceptable salt of the compound or tautomer, of any one of embodiments 68-109, wherein Ring B2 is selected from the group consisting of

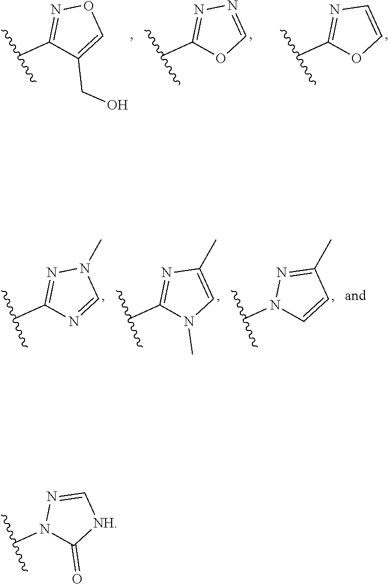

Embodiment 120. The compound or tautomer thereof, or pharmaceutically acceptable salt of the compound or tautomer, of any one of embodiments 68-109, wherein Ring B2 is selected from the group consisting of

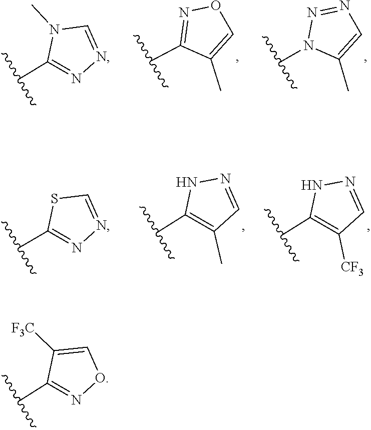

Embodiment 121. The compound or tautomer thereof, or pharmaceutically acceptable salt of the compound or tautomer, of any one of embodiments 68-109, wherein Ring B2 is 4-methyl-4H-1,2,4-triazol-3-yl.

Embodiment 122. The compound or tautomer thereof, or pharmaceutically acceptable salt of the compound or tautomer, of embodiment 68, selected from the group consisting of Compounds 282, 283, and 283b of Table 1, including “a” and “b” variants of the compounds, tautomers thereof, and pharmaceutically acceptable salts of the compounds or tautomers.

Embodiment 123. The compound or tautomer thereof, or pharmaceutically acceptable salt of the compound or tautomer, of embodiment 68, which is Compound 282, a tautomer thereof, or pharmaceutically acceptable salt of the compound or tautomer.

Embodiment 124. A pharmaceutical composition comprising a Cbl-b inhibitor of Formula (III-A):

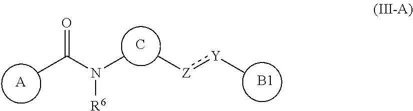

or a tautomer thereof, or a pharmaceutically acceptable salt of any of the foregoing, and a pharmaceutically acceptable excipient;

wherein Ring A,

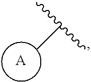

is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;

wherein Ring A is attached to the adjacent carbonyl carbon via a carbon atom of Ring A;

each R^{A} is independently selected from the group consisting of:

H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R⁶ is H, or

R⁶ and the amide group to which R⁶ is connected attach to Ring A to form a five-membered lactam ring fused to Ring A, such that the fragment

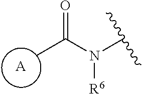

is

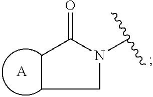

Ring C,

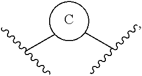

is selected from the group consisting of:

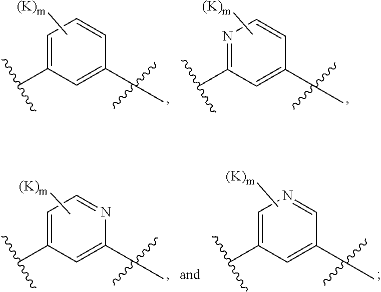

each K is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —C₁-C₈ alkyl, —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K groups is optionally substituted by one or two substituents independently selected from the group consisting of F, Cl, Br, I, —CN, —OH, Ci-C's alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m is 0, 1, or 2;

[symbol] is a single bond or a double bond,

wherein when [symbol] is a single bond, Y is C(R⁹)(R¹⁰), S, or O; and Z is C(R⁷)(R⁸), and

when [symbol] is a double bond, Y is C(R⁹); and Z is C(R⁸),

R⁷ is selected from the group consisting of H, F, —OH, C₁-C₈ alkyl optionally substituted with —OH or —O—C₁-C₈ alkyl, C₂—C's alkenyl, C₁-C₈ haloalkyl, —O—C₁-C₈ haloalkyl, and —O—C₁-C₈ alkyl optionally substituted with —OH or —O—C₁-C₈ alkyl,

R⁸ is selected from the group consisting of H, F, —OH, C₁-C₈ alkyl optionally substituted with —OH or —O—C₁-C₈ alkyl, C₂—C's alkenyl, C₁-C₈ haloalkyl, and —O—C₁-C₈ alkyl optionally substituted with —OH or —O—C₁-C₈ alkyl, or

when Y is C(R⁹)(R¹⁰) or C(R⁹), R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl, or

R⁷ and R⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

R⁹ and R¹⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R⁹ is taken together with R⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; or

R⁹ and R¹⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and

Ring B,

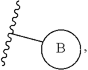

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 125. The pharmaceutical composition of embodiment 124, wherein the compound of Formula (III-A) is a compound of Formula (III):

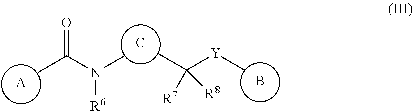

or a tautomer thereof, or a pharmaceutically acceptable salt of any of the foregoing, and a pharmaceutically acceptable excipient;

wherein Ring A,

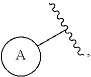

is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;

wherein Ring A is attached to the adjacent carbonyl carbon via a carbon atom of Ring A;

each R^{A} is independently selected from the group consisting of:

H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one two, or three substituents independently selected from the group consisting of —OH, oxo, C₁-C₈ alkyl, —O—C₁-C₈ alkyl, C₃-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R⁶ is H, or

R⁶ and the amide group to which R⁶ is connected attach to Ring A to form a five-membered lactam ring fused to Ring A, such that the fragment

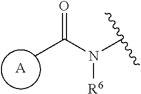

is

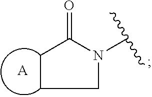

Ring C,

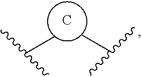

is selected from the group consisting of:

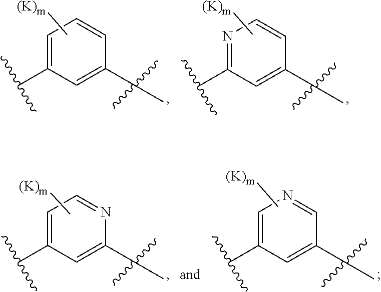

each K is independently selected from the group consisting of:

, Cl, Br, I, —CN, —OH, —C₁-C₈ alkyl optionally substituted with —OH or —O—C₁-C₈ alkyl, —C₃-C₈ cycloalkyl optionally substituted with —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with Ci-C's alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, and four- to eight-membered heterocyclyl optionally substituted with —OH or C₁-C₄ alkyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, Ci-C's hydroxyalkyl, —CN, or —O—C₁-C₈ alkyl;

or two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m is 0, 1, or 2;

R⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R⁸ is selected from the group consisting of H, F, —OH, C₁-C₈ alkyl optionally substituted with —OH or —O—C₁-C₈ alkyl, C₂-C₈ alkenyl, C₁-C₈ haloalkyl, and —O—C₁-C₈ alkyl optionally substituted with —OH or —O—C₁-C₈ alkyl, or

when Y is C(R⁹)(R¹⁰), R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl, or

R⁷ and R⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

Y is C(R⁹)(R¹⁰) or S;

R⁹ and R¹⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R⁹ is taken together with R⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; or

R⁹ and R¹⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and

Ring B,

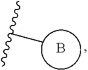

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 126. The pharmaceutical composition of embodiment 124 or embodiment 125, wherein Ring A,

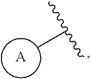

is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;

wherein Ring A is attached to the adjacent carbonyl carbon via a carbon atom of Ring A;

each R^{A} is independently selected from the group consisting of:

H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{a}R^{b} where R^{a} and R^{b} are independently H, C₁-C₈ alkyl, C₁-C₈ haloalkyl, or C₃-C₈ cycloalkyl, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, or I, —(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R⁶ is H, or

R⁶ and the amide group to which R⁶ is connected attach to Ring A to form a five-membered lactam ring fused to Ring A, such that the fragment

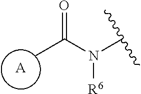

is

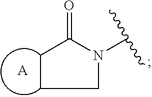

Ring C,

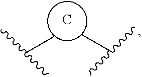

is selected from the group consisting of:

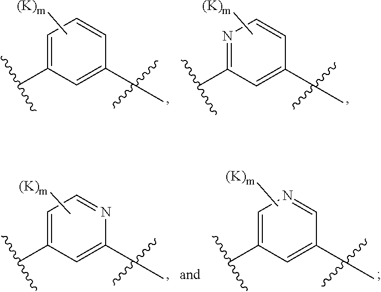

each K is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl,

—O—C₁-C₈ haloalkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m is 0, 1, or 2;

R⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,

R⁸ is selected from the group consisting of H, F, —OH, C₁-C₈ alkyl optionally substituted with —OH or —O—C₁-C₈ alkyl, C₂-C₈ alkenyl, C₁-C₈ haloalkyl, and —O—C₁-C₈ alkyl optionally substituted with —OH or —O—C₁-C₈ alkyl, or

when Y is C(R⁹)(R¹⁰), R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl, or

R⁷ and R⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and

Y is C(R⁹)(R¹⁰) or S;

R⁹ and R¹⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or

R⁹ is taken together with R⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; and

Ring B,

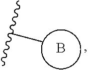

is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 127. The pharmaceutical composition of any one of embodiments 124-126, wherein ring A is

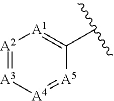

wherein

A¹ is CR¹ or N,

A² is CR² or N,

A³ is CR³ or N,

A⁴ is CR⁴ or N, and

A⁵ is CR⁵ or N,

wherein no more than two of A¹, A², A³, A⁴, and A⁵ are N; and R¹, R², R³, R⁴, and R⁵ are each independently selected from R^{A}.

Embodiment 128. The pharmaceutical composition of embodiment 127, wherein at least one of R¹, R², R³, and R⁴ is selected from the group consisting of: a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 129. The pharmaceutical composition of embodiment 127, wherein at least one of R¹, R², R³, and R⁴ is selected from the group consisting of: —C₁-C₈ haloalkyl-OH, —C₁-C₈ haloalkyl-COOH, —CO(C₁-C₈ alkyl), a nine-membered heterocyclic ring, —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), and a —O-(four- to eight-membered heterocyclic ring), wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring.

Embodiment 130. The pharmaceutical composition of embodiment 127 or embodiment 128, wherein R² is a —(C₁-C₄ alkylene)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 131. The pharmaceutical composition of embodiment 127 or embodiment 128, wherein R² is a —(C₁-C₂ alkylene)-(four-membered heterocyclic ring), a —(C₁-C₂ alkylene)-(five-membered heterocyclic ring), or a —(C₁-C₂ alkylene)-(six-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 132. The pharmaceutical composition of embodiment 127 or embodiment 128, wherein R² is a —CH₂-(four- to eight-membered heterocyclic ring) or a —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 133. The pharmaceutical composition of embodiment 127 or embodiment 128, wherein R² is —CH₂-(pyrrolidinyl) or —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 134. The pharmaceutical composition of embodiment 127 or embodiment 128, wherein R² is —CH₂-(azetidinyl) or —CH(CH₃)-azetidinyl, wherein the azetidinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 135. The pharmaceutical composition of any one of embodiments 127-134, wherein R⁴ is selected from the group consisting of H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—Cᵣ C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl.

Embodiment 136. The pharmaceutical composition of any one of embodiments 127-134, wherein R⁴ is CF₃.

Embodiment 137. The pharmaceutical composition of any one of embodiments 124-126, wherein ring A is selected from a five- to ten-membered heterocyclic ring system optionally substituted with one, two, or three independently chosen R^{A} groups or a five- to ten-membered heteroaryl ring system optionally substituted with one, two, or three independently chosen R^{A} groups.

Embodiment 138. The pharmaceutical composition of any one of embodiments 124-126, wherein ring A is selected from the group consisting of pyridyl, quinolinyl, isoquinolinyl, quinoxalinyl, cinnolinyl, quinazolinyl, naphthyridinyl, benzoxazolyl, benzothiazolyl, benzoimidazoyl, pyrrolyl, pyrazolyl, imidazolyl, triazolyl, tetrazolyl, furanyl, isoxazolyl, oxazolyl, oxadiazolyl, thiophenyl, isothiazolyl, thiazolyl, thiadiazolyl, pyridazinyl, pyrimidinyl, pyrazinyl, triazinyl, tetrazinyl, indolyl, isoindolyl, indazolyl, benzotriazolyl, benzofuranyl, benzoisoxazolyl, benzoxadiazolyl, benzothiophenyl, benzoisothiazolyl, benzothiadiazolyl, pyrrolopyridinyl, pyrazolopyridinyl, imidazopyridinyl, triazolopyridinyl, furopyridinyl, oxazolopyridinyl, isoxazolopyridinyl, oxadiazolopyridinyl, thienopyridinyl, thiazolopyridinyl, isothiazolopyridinyl, thiadiazolopyridinyl, thienopyridinyl, phthalazinyl, pyrazolothiazolyl, pyrazolothiazolyl imidazothiazolyl, tetrahydroquinolinyl, tetrahydroisoquinolinyl, decahydroquinolinyl, decahydroisoquinolinyl, indolinyl, isoindolinyl, tetrahydronaphthyridinyl and hexahydrobenzoimidazolyl, each optionally substituted with one, two, or three independently chosen R^{A} groups.

Embodiment 139. The pharmaceutical composition of any one of embodiments 124-126, wherein ring A is selected from the group consisting of quinolinyl optionally substituted with one, two, or three independently chosen R^{A} groups, isoquinolinyl optionally substituted with one, two, or three independently chosen R^{A} groups, 1H-benzo[d]imidazolyl optionally substituted with one, two, or three independently chosen R^{A} groups, and indolyl optionally substituted with one, two, or three independently chosen R^{A} groups.

Embodiment 140. The pharmaceutical composition of any one of embodiments 124-126, wherein ring A is selected from the group consisting of

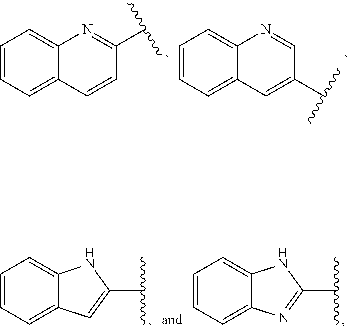

each optionally substituted with one, two, or three independently chosen R^{A} groups.

Embodiment 141. The pharmaceutical composition of any one of embodiments 124-126, wherein ring A is selected from the group consisting of

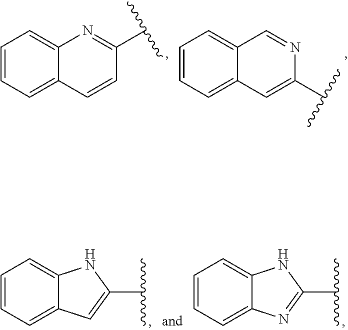

each optionally substituted with one, two, or three substituents independently selected from the group consisting of

F, Cl, Br, I, —CN, C₁-C₈ alkyl, —O—C₁-C₈ alkyl, C₁-C₈ haloalkyl, —O—C₁-C₈ alkyl-heteroaryl,

a three- to eight-membered heterocyclic ring or a five- to eight-membered heteroaryl ring, each heterocyclic ring or heteroaryl ring containing one, two, or three heteroatoms independently selected from the group consisting of O, N, and S and optionally substituted with —OH, oxo, C₁-C₈ alkyl, or —C(═O)—C₁-C₈ alkyl, and

—O—(C₁-C₄ alkylene)-C(═O)NR^{c}R^{d}, wherein R^{c} and R^{d} are independently H or C₁-C₈ alkyl.

Embodiment 142. The pharmaceutical composition of any one of embodiments 124-126, wherein ring A is selected from the group consisting of

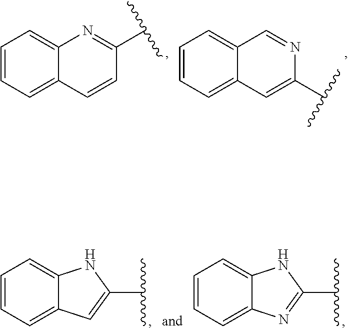

each optionally substituted with one, two, or three substituents independently selected from the group consisting of chloro, fluoro, methyl, isopropyl, cyclopropyl, methoxy, CF₃, CN, propan-2-ol, and —O—CH₂—C(═O)NHCH₃.

Embodiment 143. The pharmaceutical composition of any one of embodiments 124-126, wherein ring A is selected from the group consisting of pyridine optionally substituted with one, two, or three independently chosen R^{A} groups, pyrimidine optionally substituted with one, two, or three independently chosen R^{A} groups, and pyrazine optionally substituted with one, two, or three independently chosen R^{A} groups.

Embodiment 144. The pharmaceutical composition of any one of embodiments 124-126, wherein ring A is selected from the group consisting of

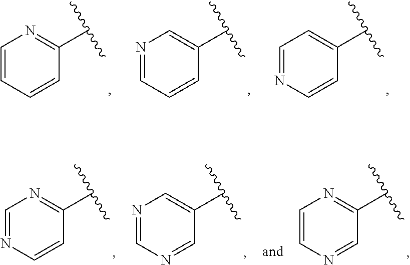 [SMILES: *c1ccccn1 ; *c1cccnc1 ; *c1ccncc1 ; *c1ccncn1 ; *c1cncnc1 ; *c1cnccn1]

each optionally substituted with one, two, or three independently chosen R^{A} groups.

Embodiment 145. The pharmaceutical composition of any one of embodiments 124-126, wherein ring A is selected from the group consisting of

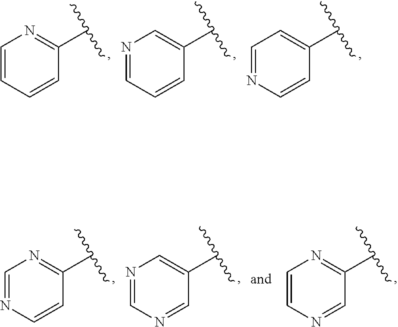

each optionally substituted with one, two, or three groups independently selected from the group consisting of

F, Cl, Br, I, —CN, C₁-C₈ alkyl, —O—C₁-C₈ alkyl, —C₁-C₈ alkyl-OH, C₁-C₈ haloalkyl, C₃-C₈ cycloalkyl, —S(═O)₂—C₁-C₈ alkyl, —(C₁-C₄ alkylene)-NR^{c}R^{d}, and —C(═O)NR^{c}R^{d},

wherein R^{c} and R^{d} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

Embodiment 146. The pharmaceutical composition of any one of embodiments 124-126, wherein ring A is selected from C₃-C₈ cycloalkyl optionally substituted with one, two, or three independently chosen R^{A} groups, and C₆-C₁₀ aryl optionally substituted with one, two, or three independently chosen R^{A} groups.

Embodiment 147. The pharmaceutical composition of any one of embodiments 124-126, wherein ring A is selected from the group consisting of cyclopropyl, cyclobutyl, cyclopentyl, cyclohexyl, cycloheptyl, cyclooctyl, phenyl, or naphthyl, each optionally substituted with one, two, or three independently chosen R^{A} groups.

Embodiment 148. The pharmaceutical composition of any one of embodiments 124-147, wherein ring A is substituted with one, two, or three R^{A} groups selected from the group consisting of: F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, —O—C₁-C₈ alkylene-heterocyclyl-(C₁-C₂ alkylene)-(four-membered heterocyclic ring), a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 149. The pharmaceutical composition of embodiment 148, wherein ring A is substituted with an R^{A} group selected from H, F, Cl, Br, I, —CN, —OH, C₁-C₈ alkyl, C₃-C₈ cycloalkyl, —O—C₁-C₈ alkyl, —C₁-C₈ alkyl-OH, and C₁-C₈ haloalkyl.

Embodiment 150. The pharmaceutical composition of embodiment 148 or embodiment 149, wherein ring A is substituted with an R^{A} group selected from —CH₂-(four- to eight-membered heterocyclic ring) and —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 151. The pharmaceutical composition of embodiment 148 or embodiment 149, wherein ring A is substituted with an R^{A} group selected from —CH₂-(pyrrolidinyl) and —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 152. The pharmaceutical composition of embodiment 148 or embodiment 149, wherein ring A is substituted with an R^{A} group selected from —CH₂-(azetidinyl) and —CH(CH₃)-azetidinyl, wherein the azetidinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

Embodiment 153. The pharmaceutical composition of any one of embodiments 124-126, wherein ring A is phenyl, optionally substituted with one, two, or three groups independently selected from the group consisting of F, Cl, Br, I, —CN, C₁-C₈ alkyl, —O—C₁-C₈ alkyl, —C₁-C₈ alkyl-OH, C₃-C₈ cycloalkyl, C₁-C₈ haloalkyl, and —C(═O)NR^{c}R^{d}, wherein R^{c} and R^{d} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

Embodiment 154. The pharmaceutical composition of any one of embodiments 124-126, wherein ring A is selected from the group consisting of

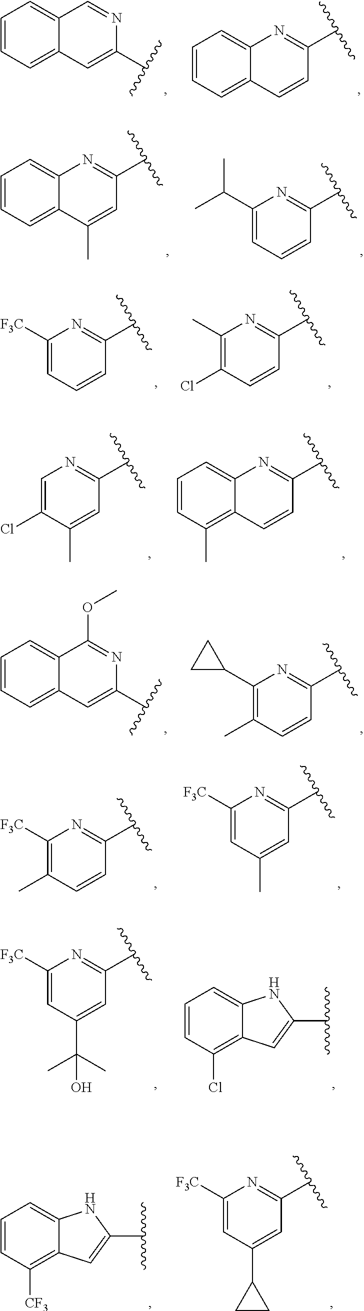

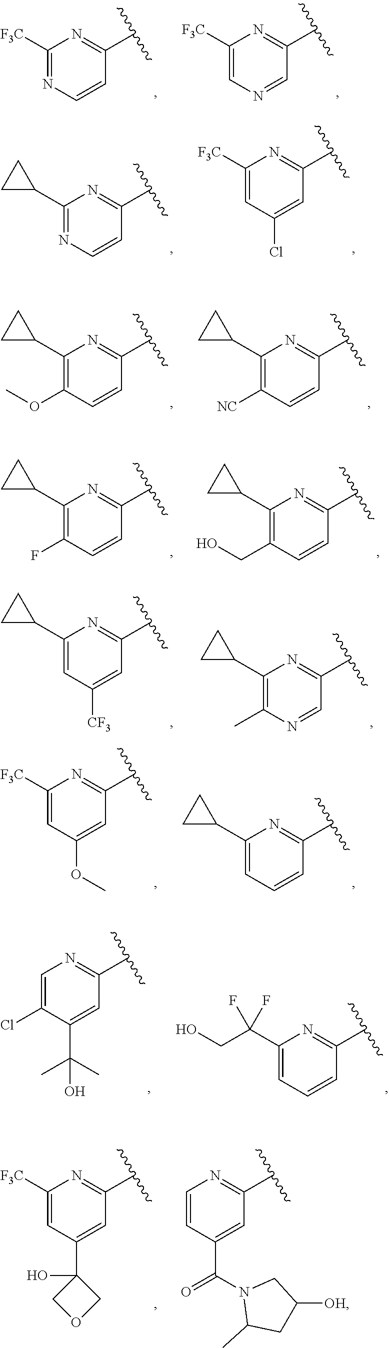

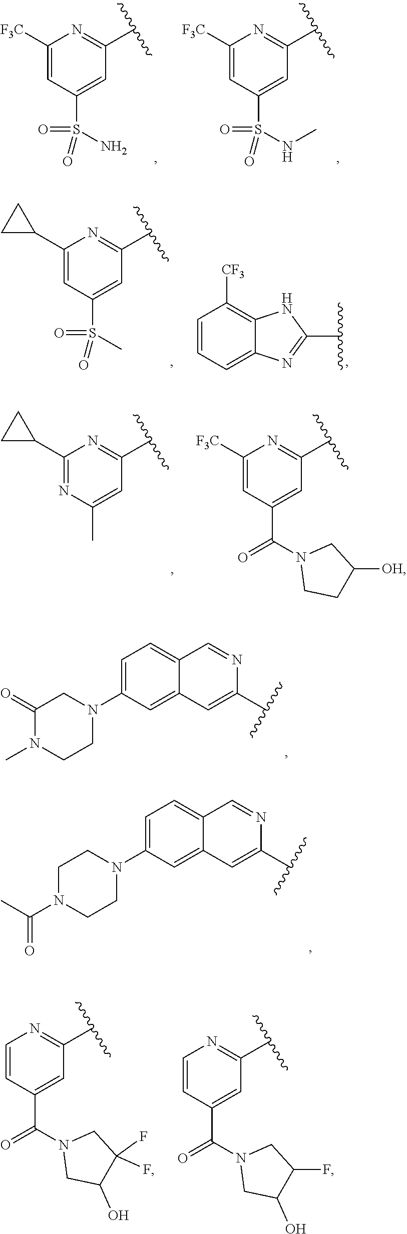

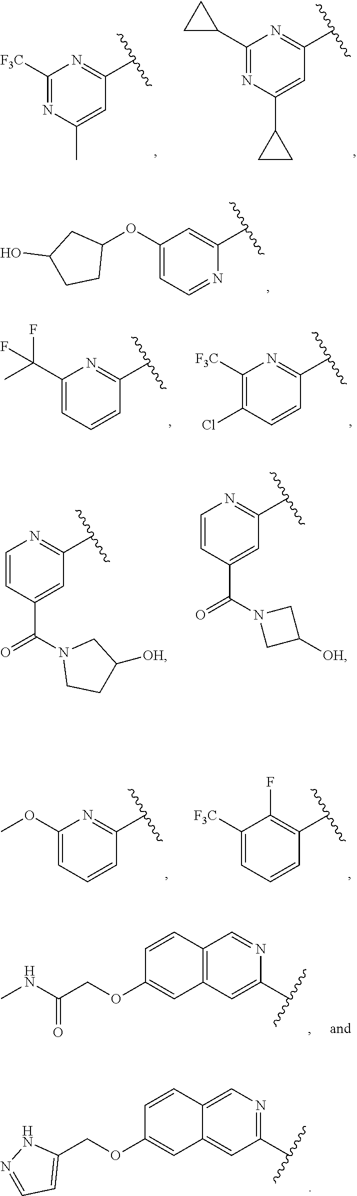

Embodiment 155. The pharmaceutical composition of any one of embodiments 124-126, wherein ring A is selected from the group consisting of

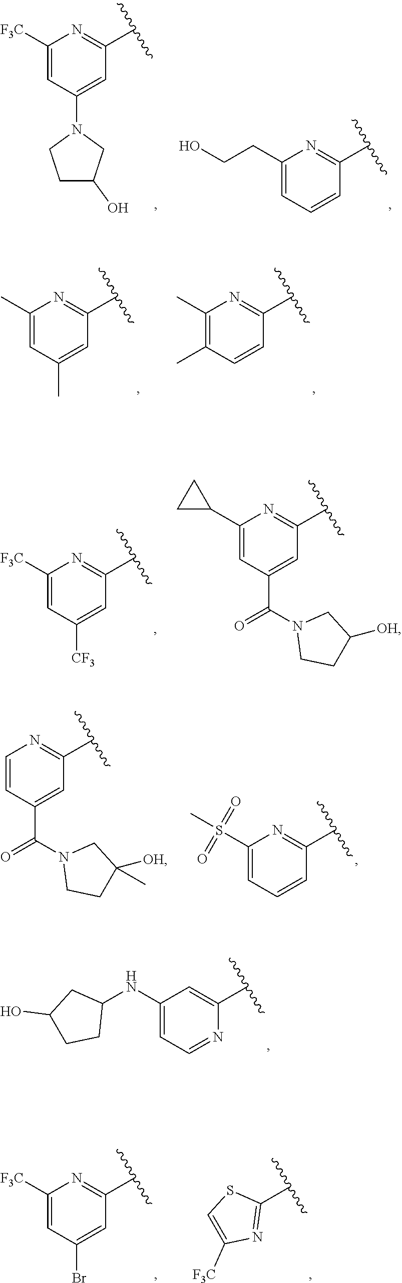

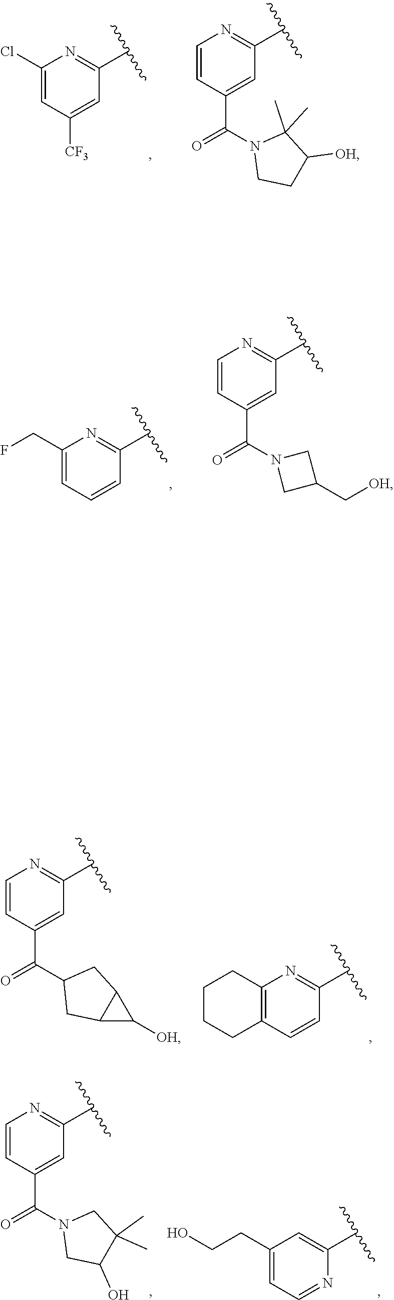

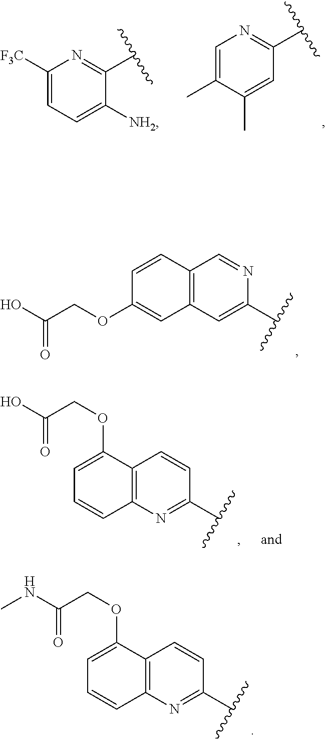

Embodiment 156. The pharmaceutical composition of any one of embodiments 124-126, wherein ring A is selected from the group consisting of

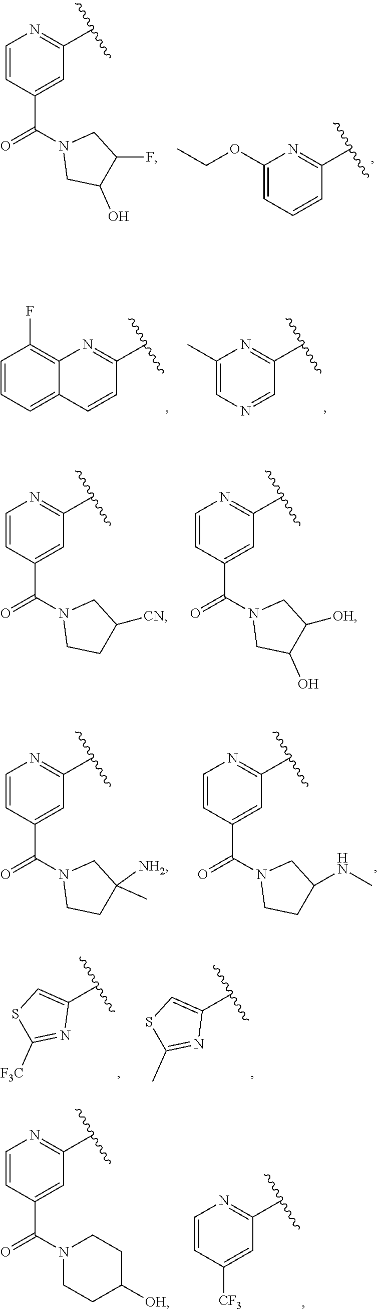

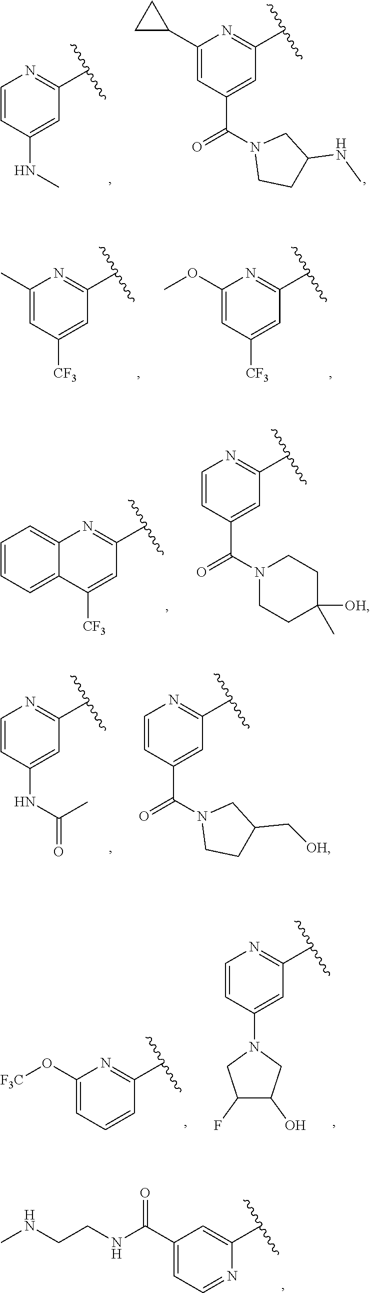

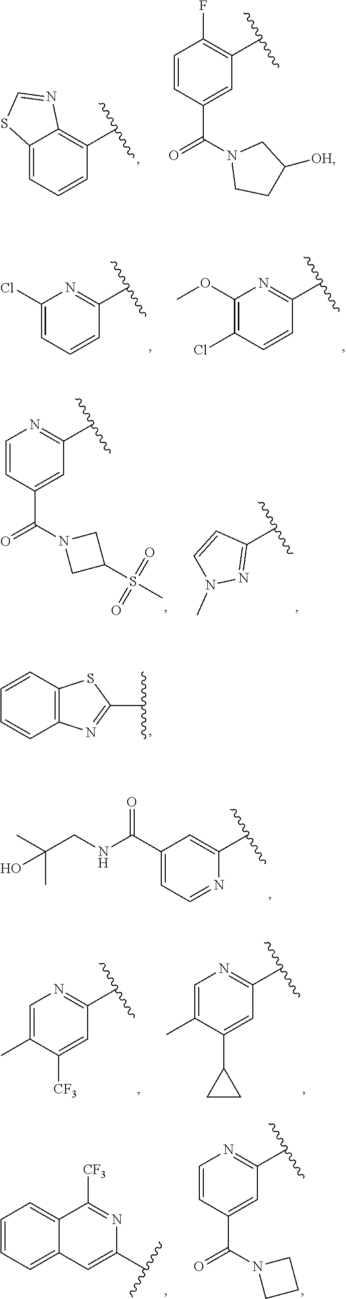

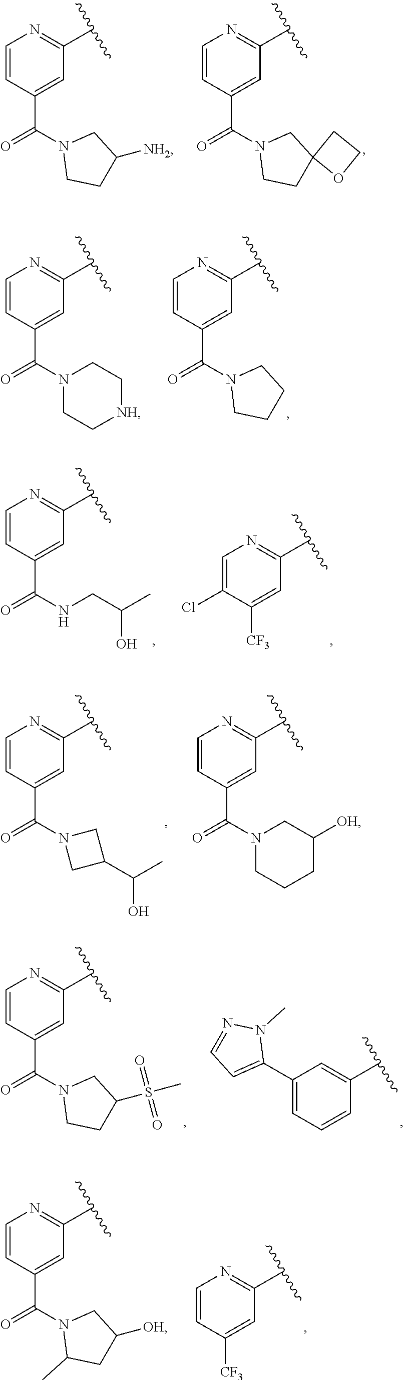

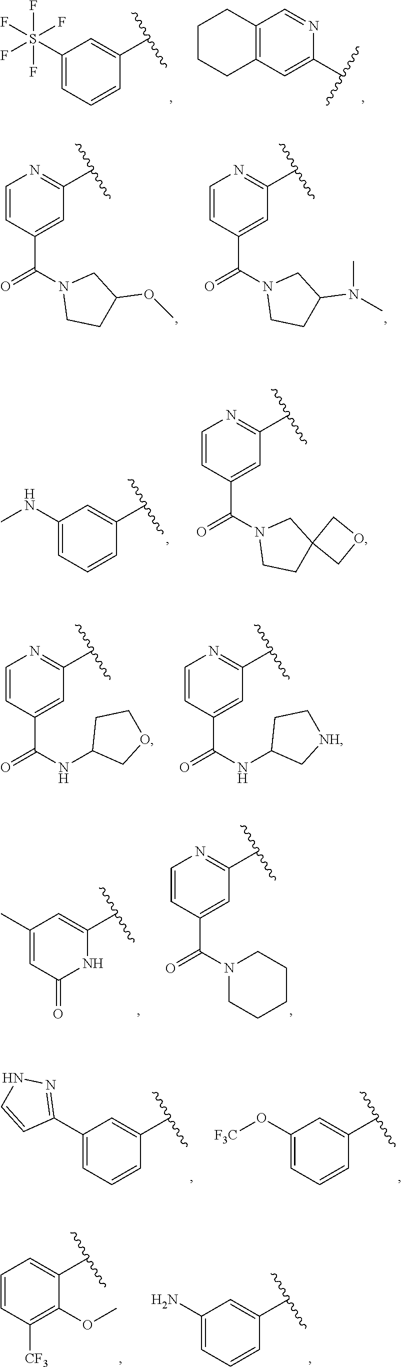

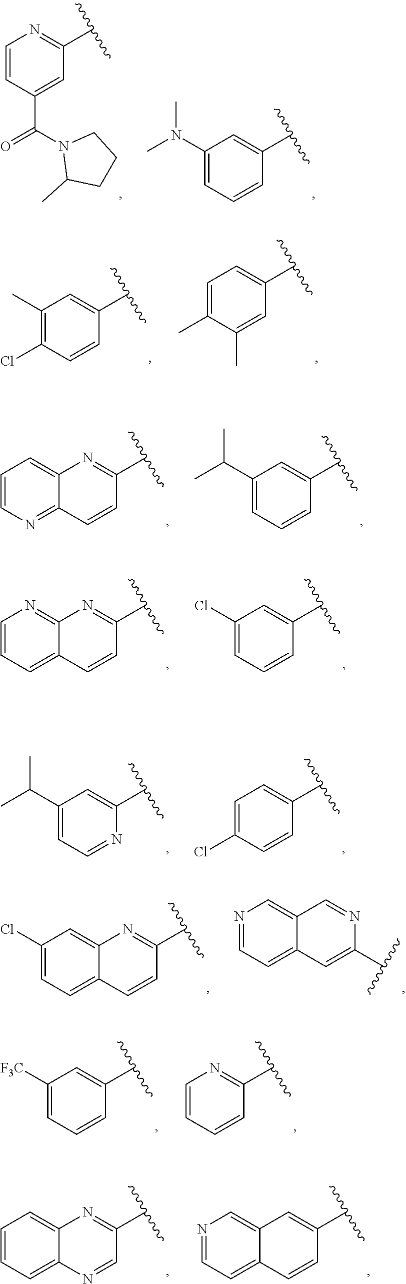

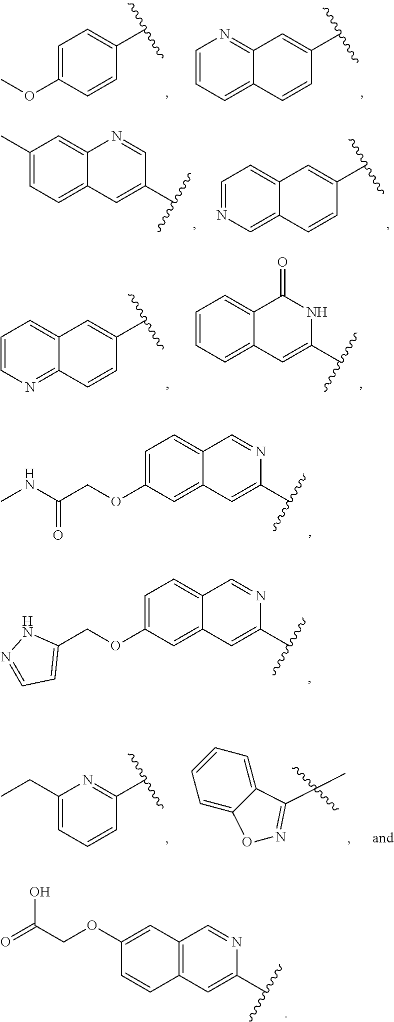

Embodiment 157. The pharmaceutical composition of any one of embodiments 124-126, wherein ring A is selected from the group consisting of

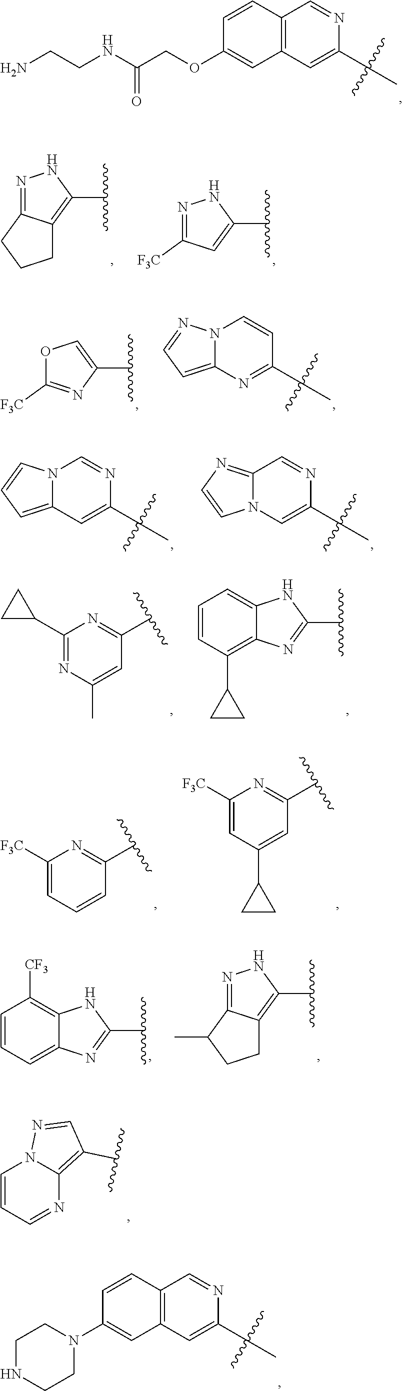

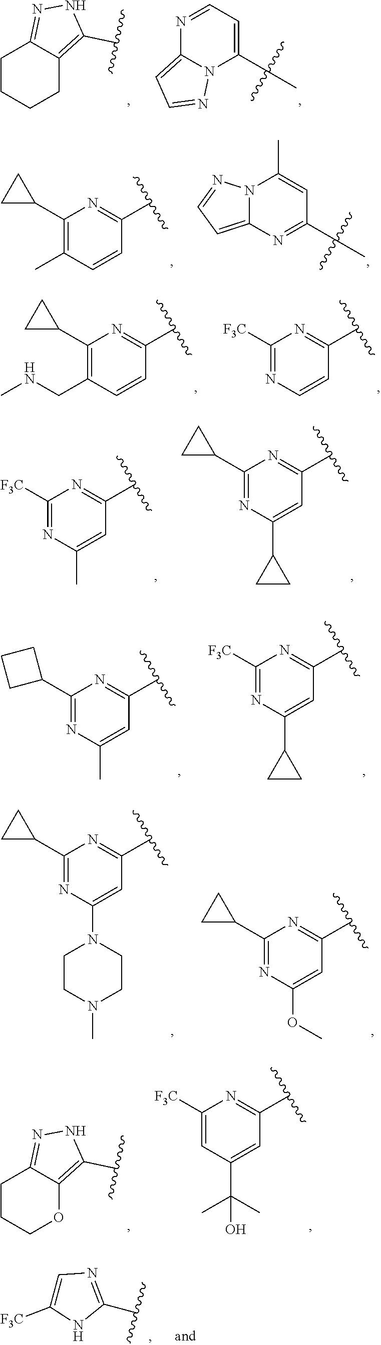

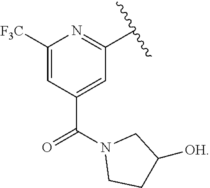 [SMILES: *c1cc(C(=O)N2CCC(O)C2)cc(C(F)(F)F)n1]

Embodiment 158. The pharmaceutical composition of any one of embodiments 124-126, wherein ring A is selected from the group consisting of

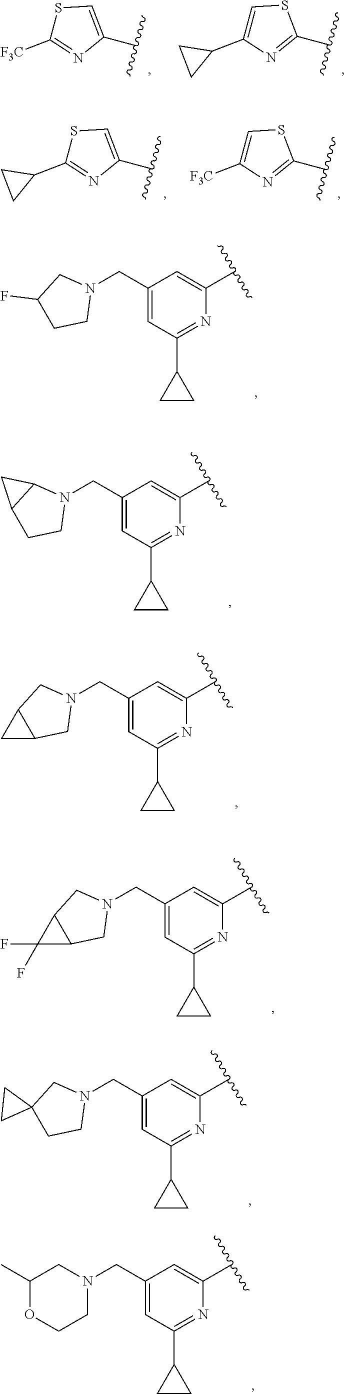

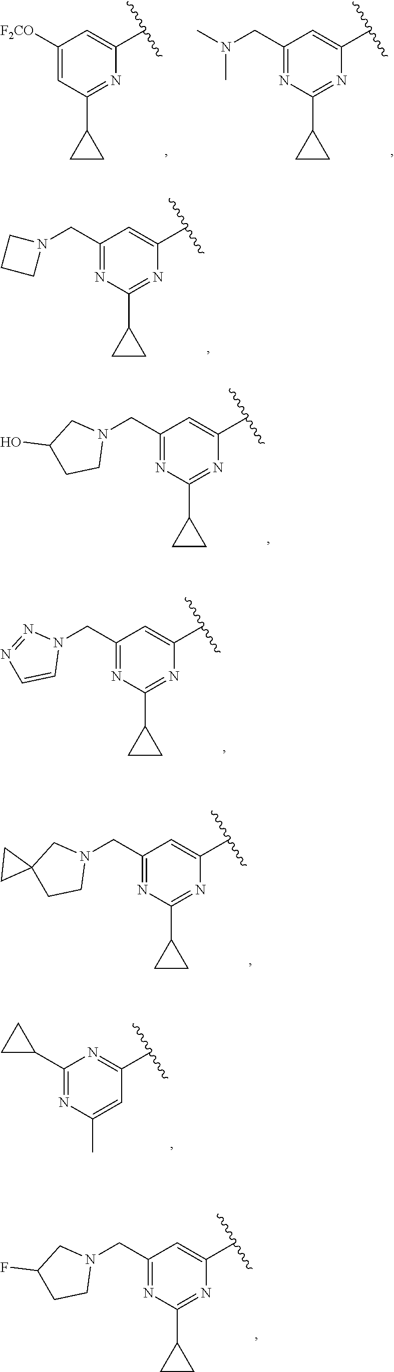

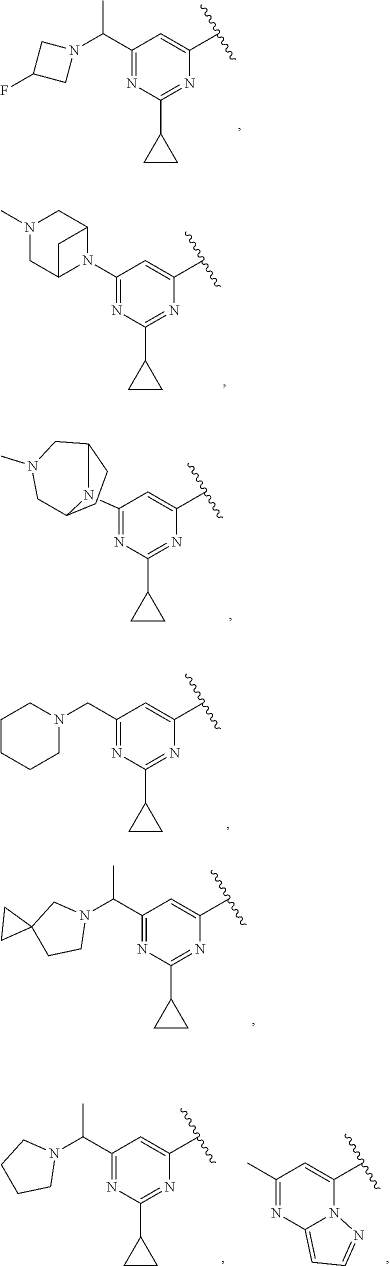

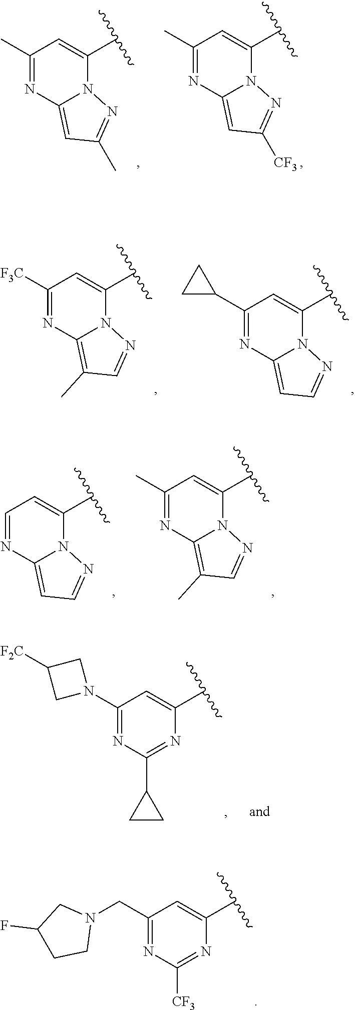

Embodiment 159. The pharmaceutical composition of any one of embodiments 124-158, wherein R⁶ is H.

Embodiment 160. The pharmaceutical composition of any one of embodiments 124-126, wherein R⁶ and the amide group to which R⁶ is connected attach to Ring A to form a five-membered lactam ring fused to Ring A, such that the fragment

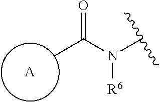

is

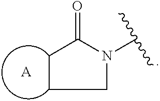

Embodiment 161. The pharmaceutical composition of embodiment 160, wherein

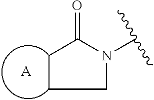

is selected from the group consisting of

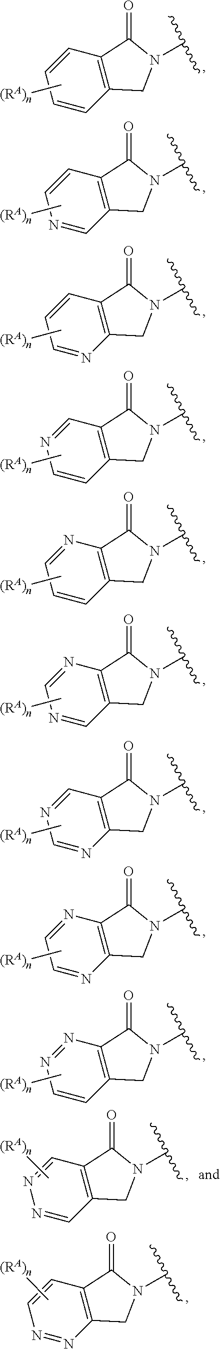

wherein:

n is 0, 1, or 2; and

each R^{A} is independently selected from the group consisting of H, F, Cl, Br, I, —OH, —CN, C₁-C₈ alkyl, —O—C₁-C₈ alkyl, —C₁-C₈ alkyl-OH, C₃-C₈ cycloalkyl, C₁-C₈ haloalkyl, —O—C₁-C₈ haloalkyl, —O—C₁-C₈ haloalkyl-OH, and —C(═O)NR^{c}R^{d}, wherein R^{c} and R^{d} are taken together with the nitrogen to which they are attached to form a three- to eight-membered heterocyclic ring optionally substituted with one, two, or three substituents independently selected from the group consisting of —OH, oxo, —CN, C₁-C₄ alkyl, —C(═O)—C₁-C₄ alkyl, F, Cl, Br, and I.

Embodiment 162. The pharmaceutical composition of embodiment 160, wherein

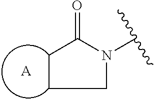

is

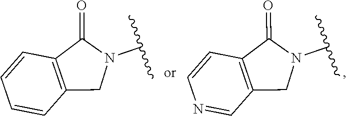 [SMILES: *N1Cc2ccccc2C1=O ; *N1Cc2cnccc2C1=O]

wherein ring A is optionally substituted with one, two, or three groups independently selected from the group consisting of F, Cl, Br, I, —OH, —CN, C₁-C₈ alkyl, —O—C₁-C₈ alkyl, —C₁-C₈ alkyl-OH, C₃-C₈ cycloalkyl, C₁-C₈ haloalkyl, —O—C₁-C₈ haloalkyl, —O—C₁-C₈ haloalkyl-OH, and —C(═O)NR^{c}R^{d}, wherein R^{c} and R^{d} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

Embodiment 163. The pharmaceutical composition of embodiment 160, wherein

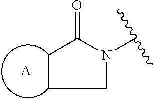

is selected from the group consisting of

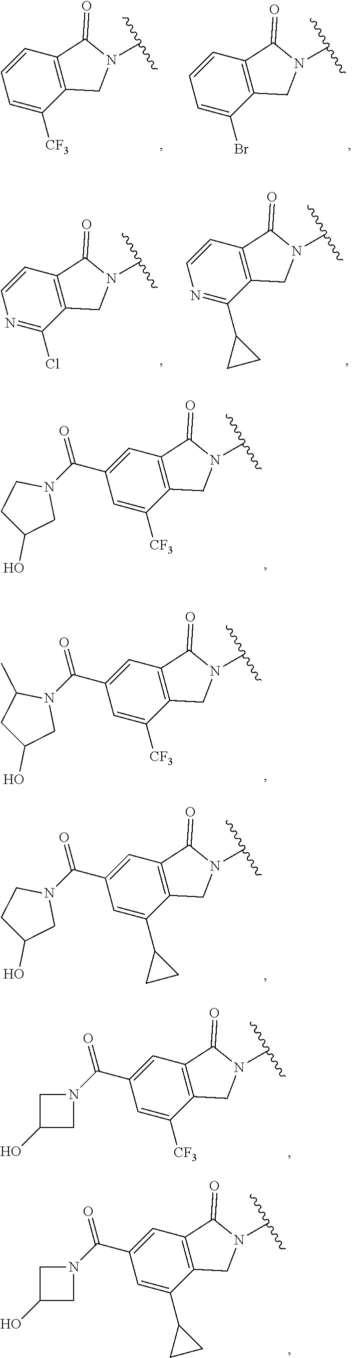

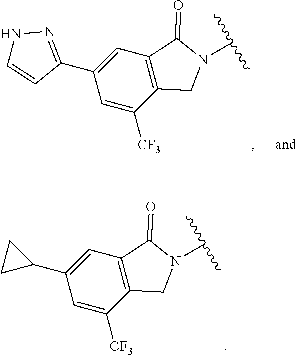 [SMILES: *N1Cc2c(cc(-c3cc[nH]n3)cc2C(F)(F)F)C1=O ; *N1Cc2c(cc(C3CC3)cc2C(F)(F)F)C1=O]

Embodiment 164. The pharmaceutical composition of embodiment 160, wherein

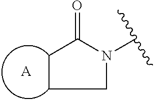

is selected from the group consisting of

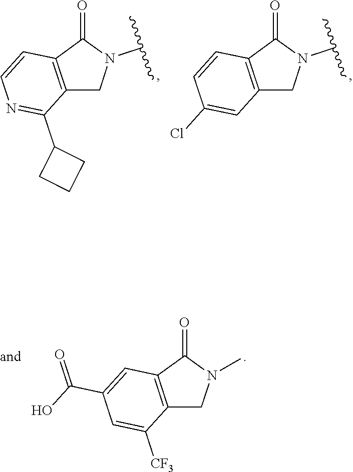 [SMILES: *N1Cc2c(ccnc2C2CCC2)C1=O ; *N1Cc2cc(Cl)ccc2C1=O ; CN1Cc2c(cc(C(=O)O)cc2C(F)(F)F)C1=O]

Embodiment 165. The pharmaceutical composition of embodiment 160, wherein

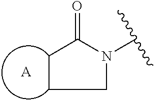

is selected from the group consisting of

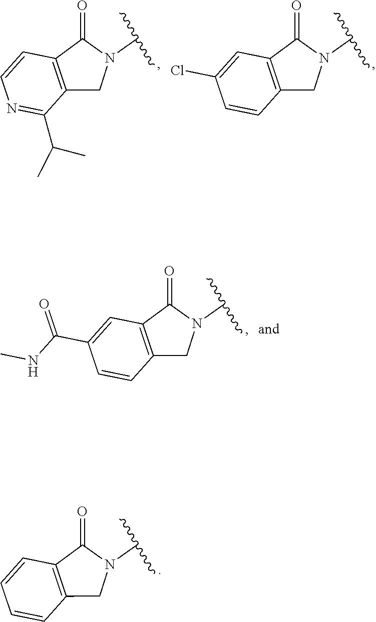 [SMILES: *N1Cc2c(ccnc2C(C)C)C1=O ; *N1Cc2ccc(Cl)cc2C1=O ; *N1Cc2ccc(C(=O)NC)cc2C1=O ; *N1Cc2ccccc2C1=O]

Embodiment 166. The pharmaceutical composition of embodiment 160, wherein

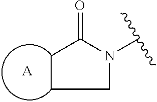

is selected from the group consisting of

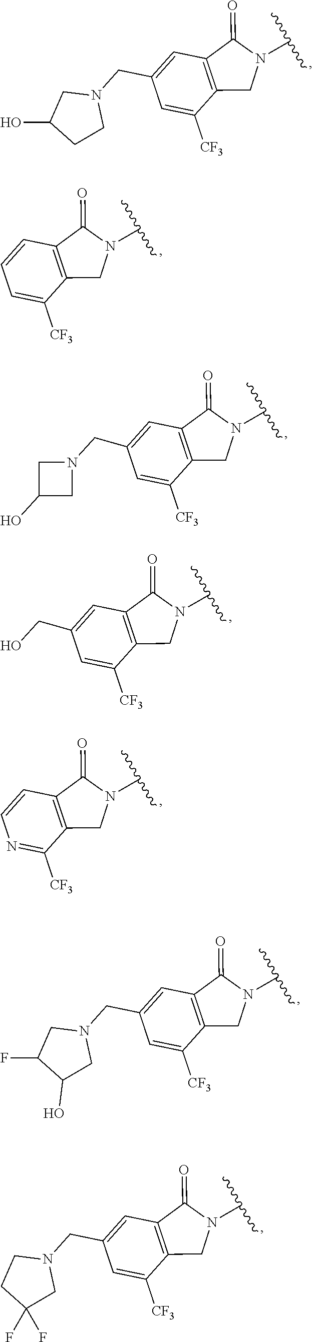

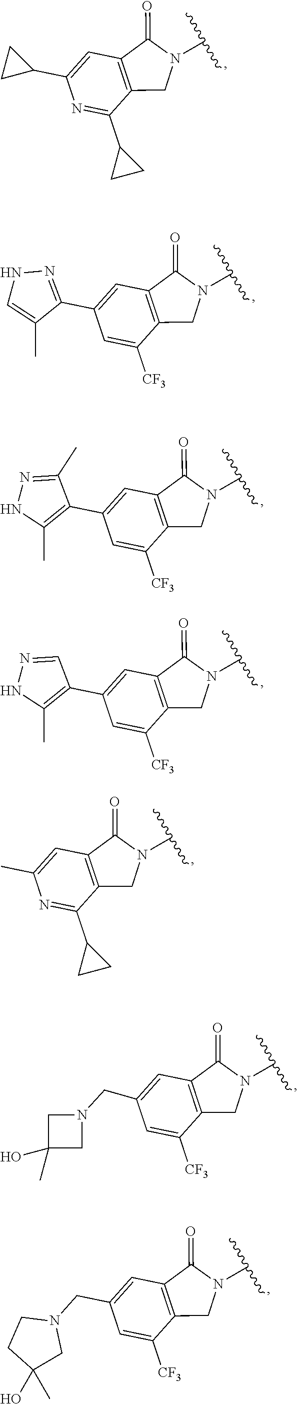

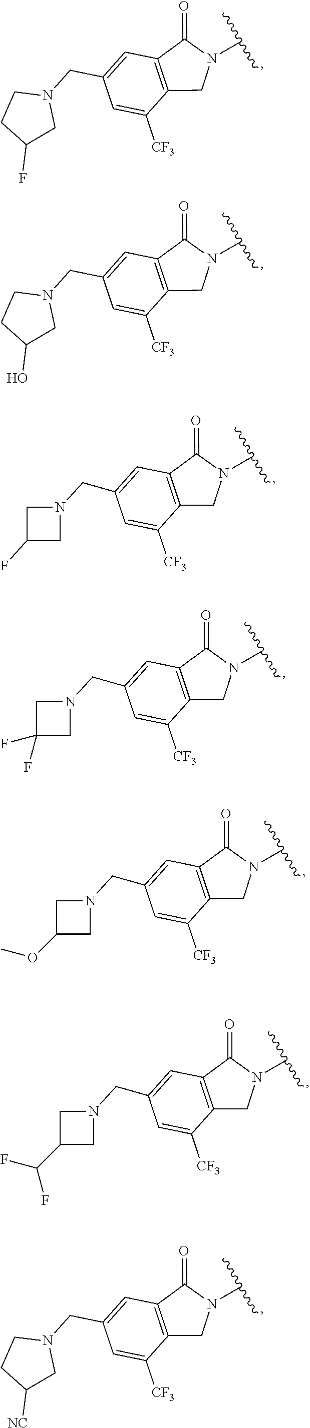

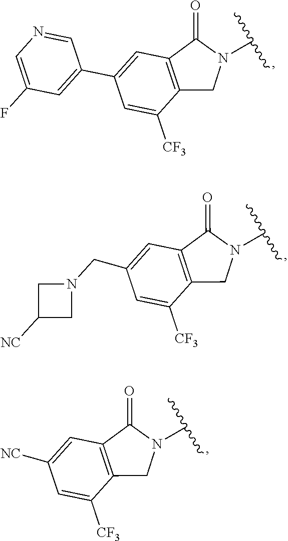 [SMILES: *N1Cc2c(cc(-c3cncc(F)c3)cc2C(F)(F)F)C1=O ; *N1Cc2c(cc(CN3CC(C#N)C3)cc2C(F)(F)F)C1=O ; *N1Cc2c(cc(C#N)cc2C(F)(F)F)C1=O]

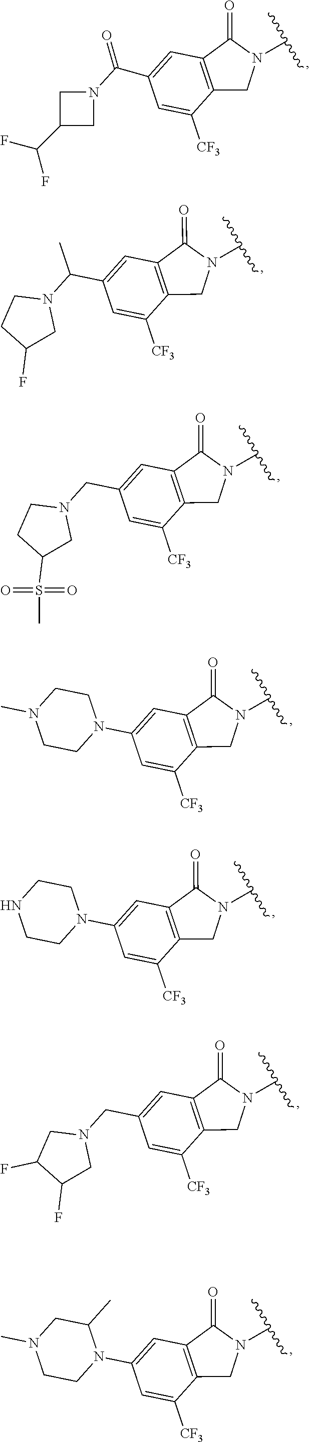

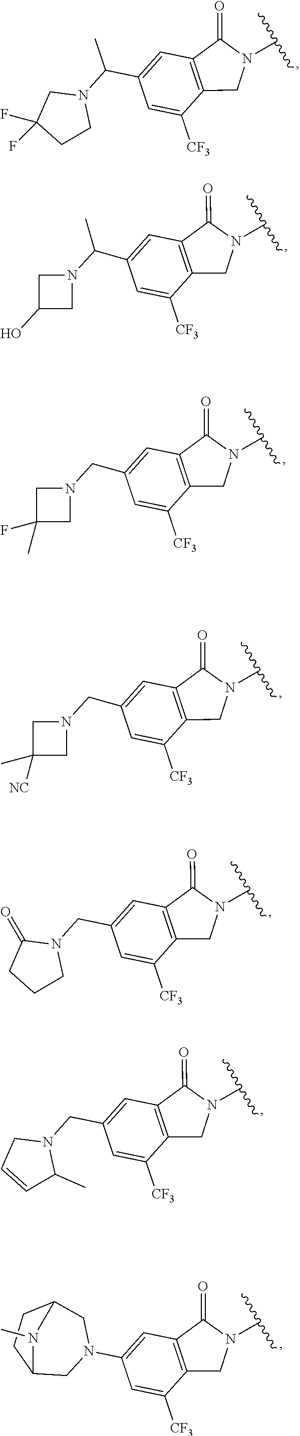

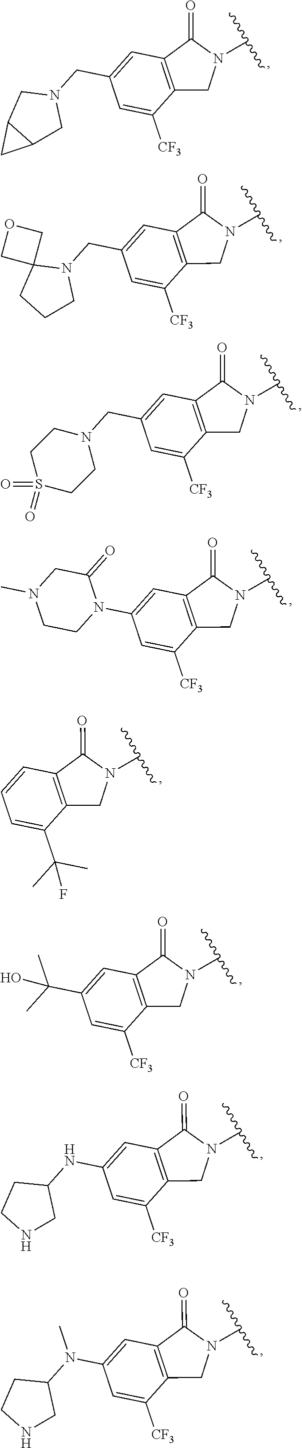

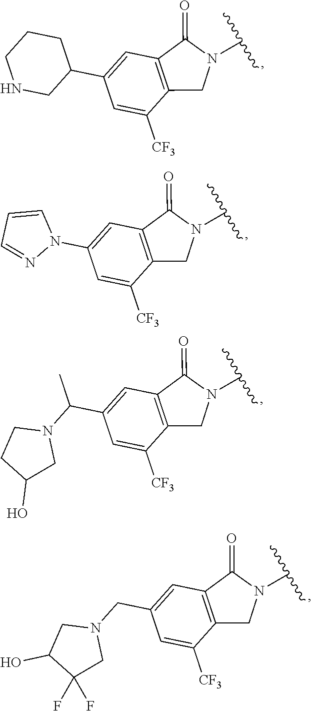

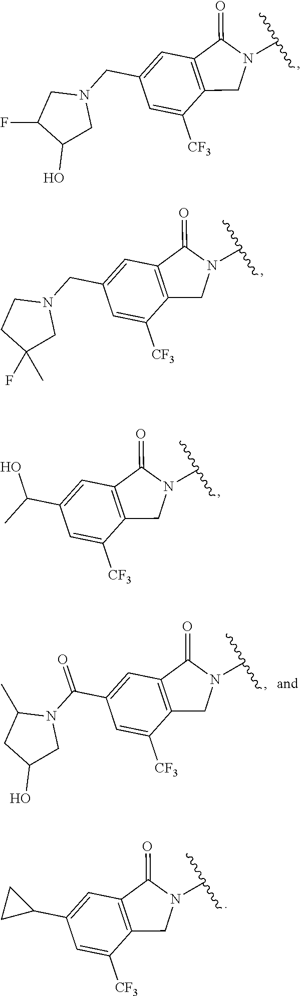 [SMILES: *N1Cc2c(cc(CN3CC(O)C(F)C3)cc2C(F)(F)F)C1=O ; *N1Cc2c(cc(CN3CCC(C)(F)C3)cc2C(F)(F)F)C1=O ; *N1Cc2c(cc(C(C)O)cc2C(F)(F)F)C1=O ; *N1Cc2c(cc(C(=O)N3CC(O)CC3C)cc2C(F)(F)F)C1=O ; *N1Cc2c(cc(C3CC3)cc2C(F)(F)F)C1=O]

Embodiment 167. The pharmaceutical composition of embodiment 160, wherein

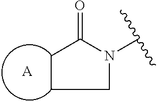

is selected from the group consisting of

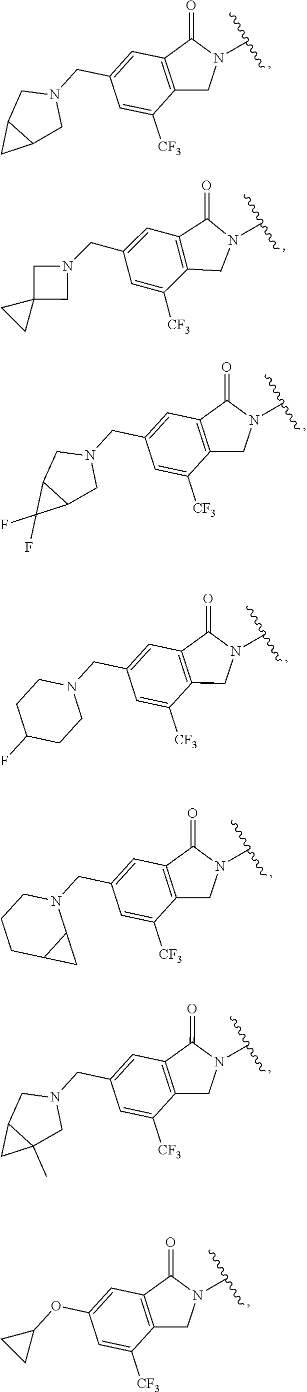

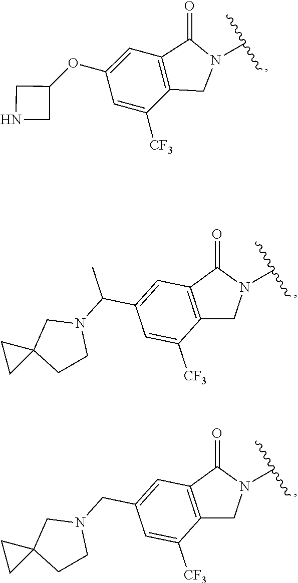 [SMILES: *N1Cc2c(cc(OC3CNC3)cc2C(F)(F)F)C1=O ; *N1Cc2c(cc(C(C)N3CCC4(CC4)C3)cc2C(F)(F)F)C1=O ; *N1Cc2c(cc(CN3CCC4(CC4)C3)cc2C(F)(F)F)C1=O]

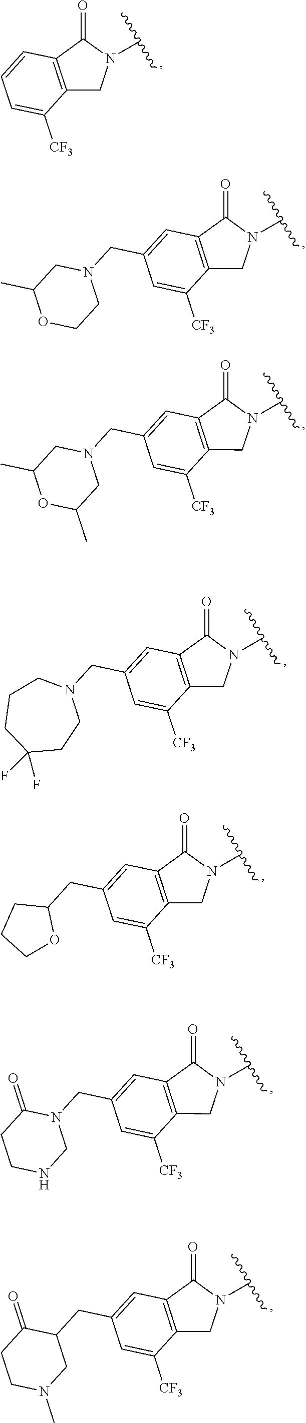

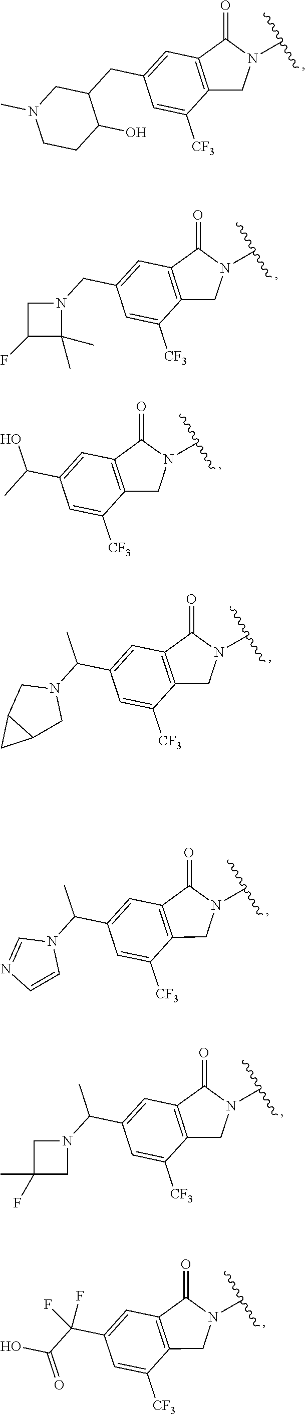

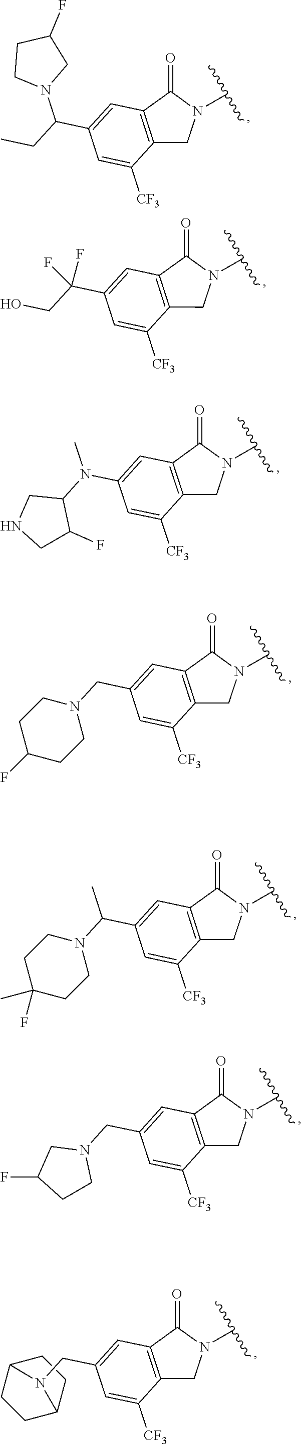

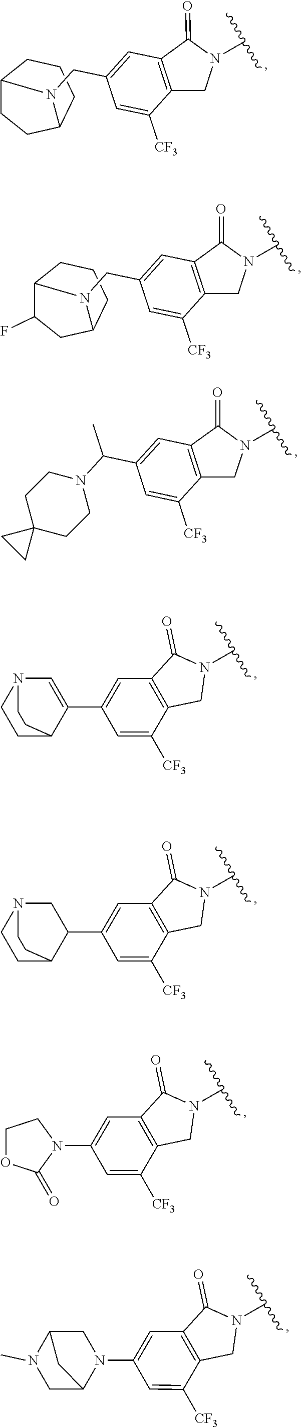

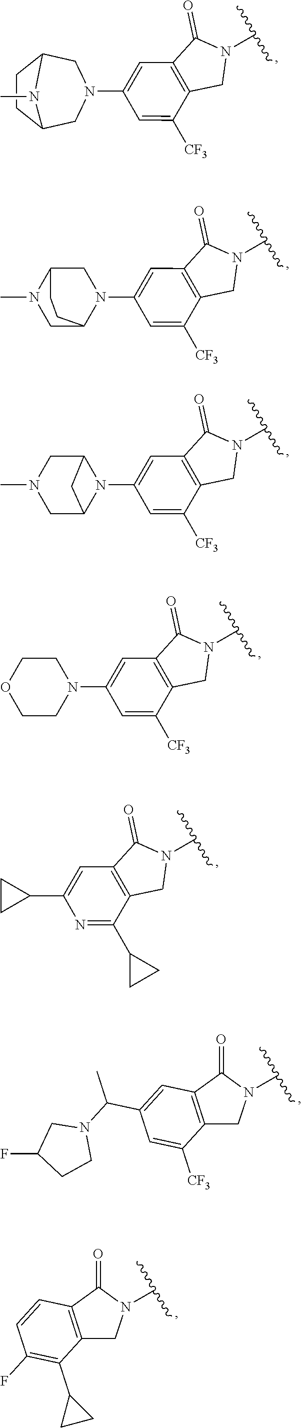

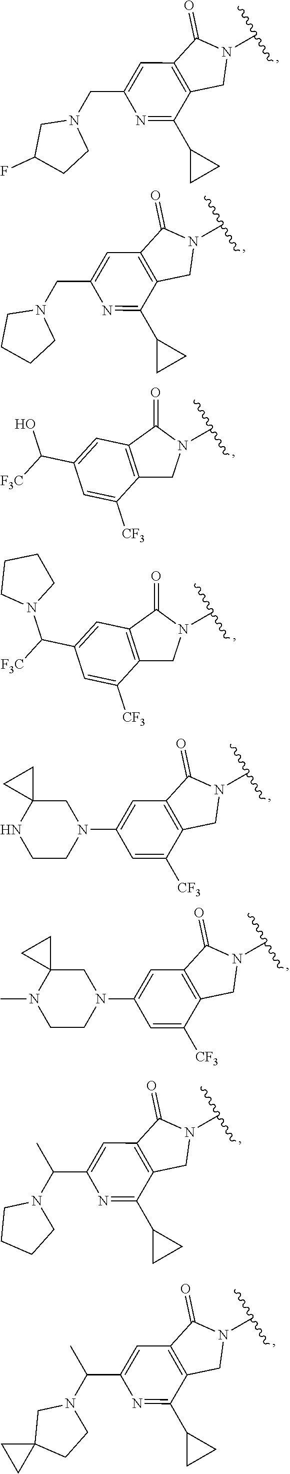

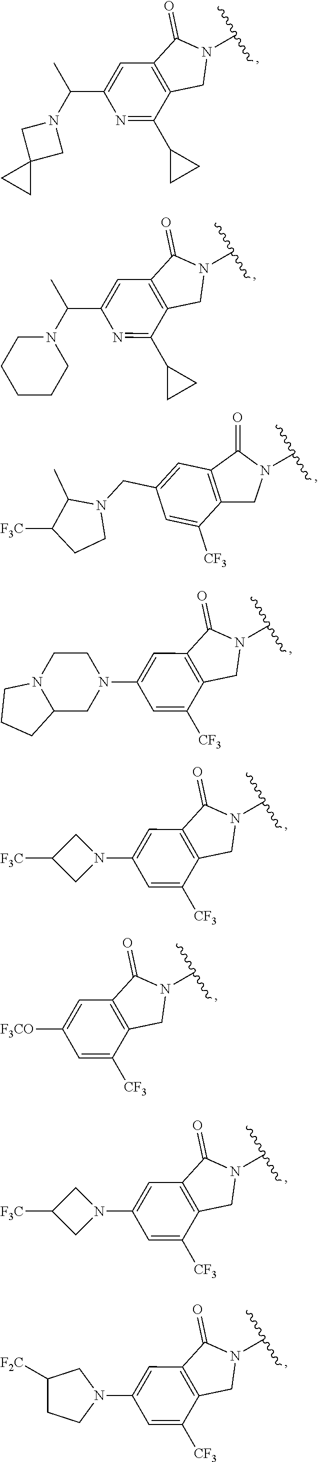

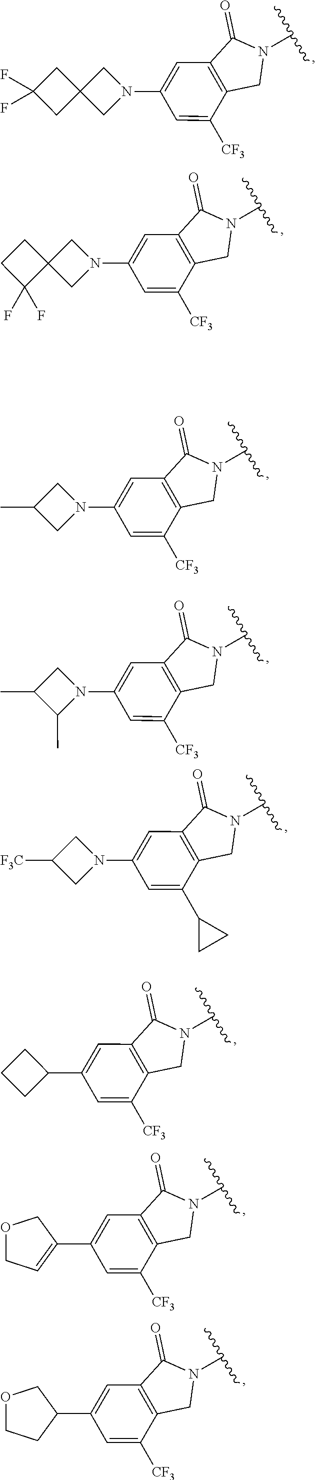

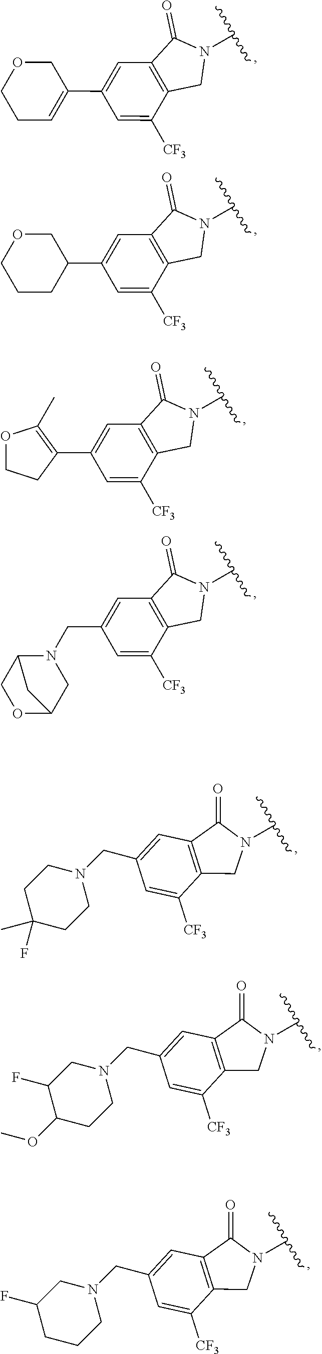

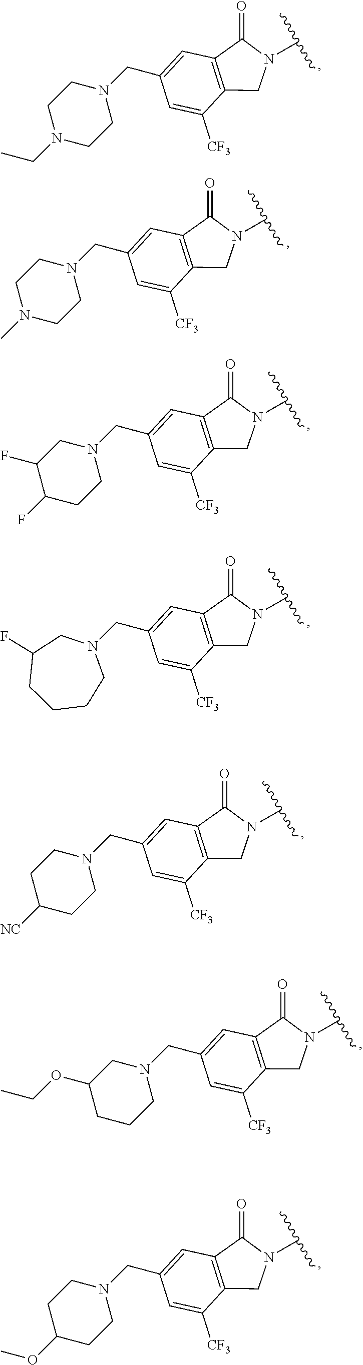

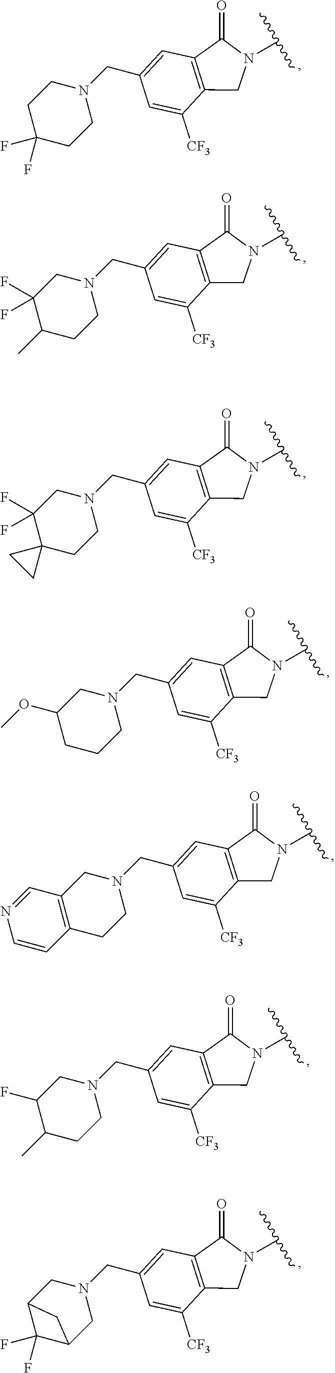

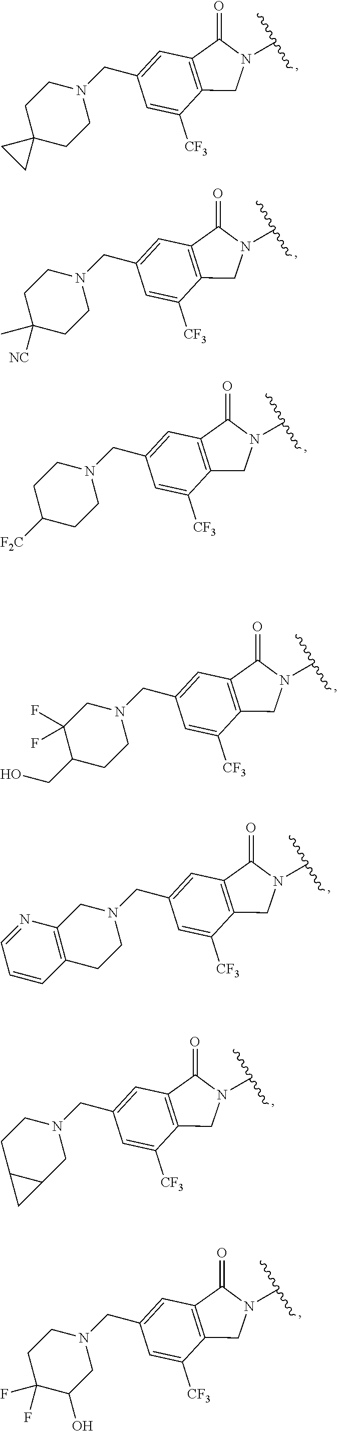

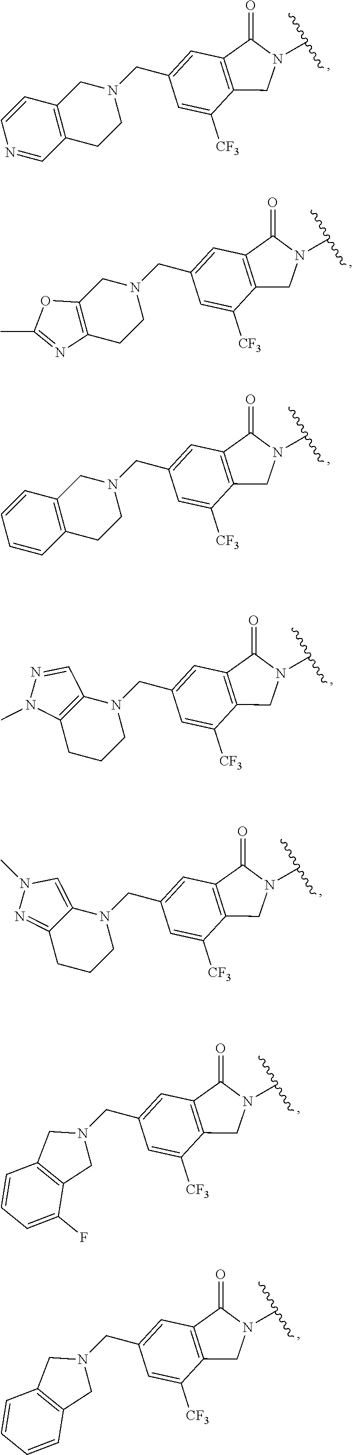

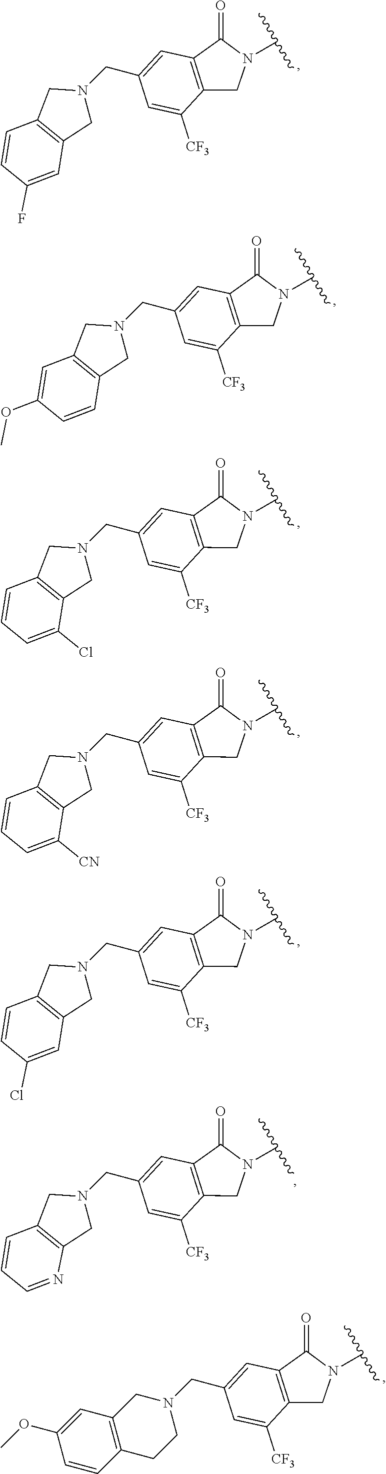

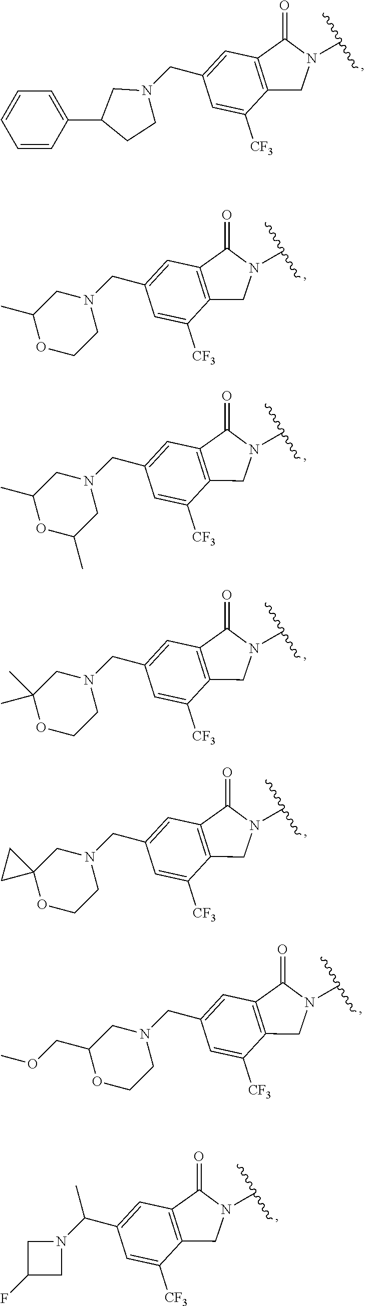

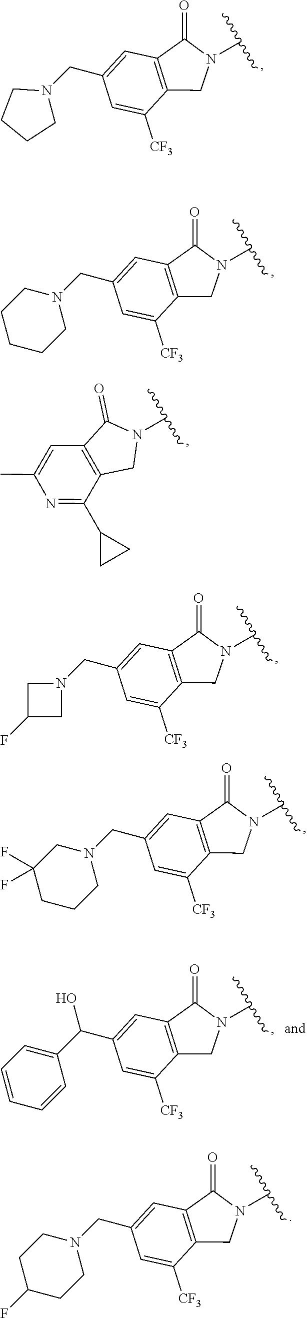

Embodiment 168. The pharmaceutical composition of any one of embodiments 124-167, wherein ring C is selected from the group consisting of

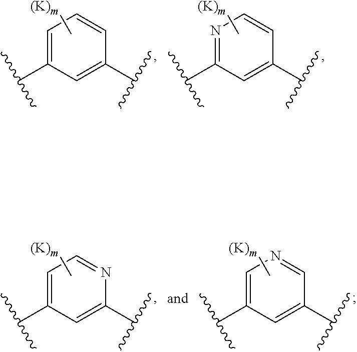

wherein K is independently selected from the group consisting of F, Cl, Br, I, —CN, C₁-C₈ alkyl optionally substituted with —OH or —O—C₁-C₈ alkyl, C₃-C₈ cycloalkyl optionally substituted with —OH or —O—C₁-C₈ alkyl, —O—C₁-C₈ alkyl, —O—C₁-C₈ haloalkyl, and —NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with OH, F, Cl, Br, I, C₁-C₈ alkyl, C₁-C₈ haloalkyl, C₁-C₈ hydroxyalkyl, —CN, or —O—C₁-C₈ alkyl; and m is 0, 1, or 2.

Embodiment 169. The pharmaceutical composition of any one of embodiments 124-167, wherein ring C is

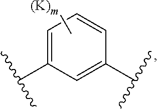

wherein m is 0, 1, or 2, and each K is independently selected from the group consisting of:

F, Cl, Br, I, —CN,

C₁-C₈ alkyl optionally substituted with —OH or —O—C₁-C₈ alkyl,

—O—C₁-C₈ alkyl,

—O—C₁-C₈ haloalkyl, and

—NR^{g}R^{h}, where R^{g} and R^{h} are independently selected from the group consisting of H, C₁-C₈ alkyl optionally substituted with —OH, and heterocyclyl.

Embodiment 170. The pharmaceutical composition of any one of embodiments 124-167, wherein ring C is

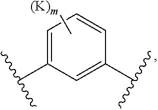

wherein m is 0, 1, or 2, and each K is independently:

C₃-C₈ cycloalkyl optionally substituted with —C₁-C₈ alkyl, —OH or —O—C₁-C₈ alkyl, or

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, Ci-C's hydroxyalkyl, —CN, or —O—C₁-C₈ alkyl.

Embodiment 171. The pharmaceutical composition of any one of embodiments 124-167, wherein ring C is selected from the group consisting of

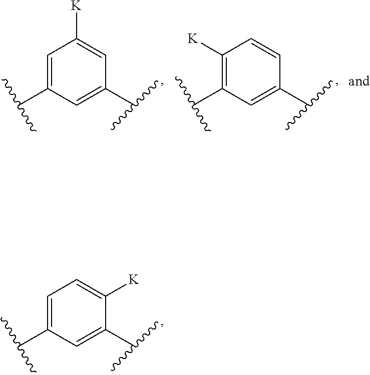

wherein K is selected from the group consisting of F, Cl, Br, I, —CN, C₁-C₈ alkyl optionally substituted with —OH or —O—C₁-C₈ alkyl, —O—C₁-C₈ alkyl, —O—C₁-C₈ haloalkyl, and —NR^{g}R^{h}, where R^{g} and R^{h} are independently selected from the group consisting of H, C₁-C₈ alkyl optionally substituted with —OH, and heterocyclyl.

Embodiment 172. The pharmaceutical composition of any one of embodiments 124-167, wherein ring C is selected from the group consisting of

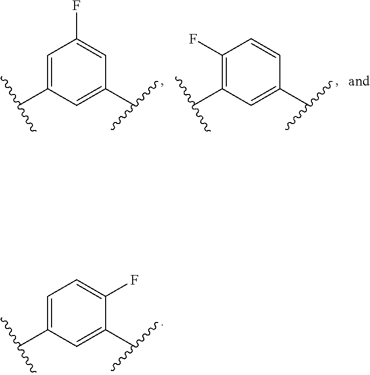 [SMILES: *c1cc(*)cc(F)c1 ; *c1ccc(F)c(*)c1 ; *c1ccc(F)c(*)c1]

Embodiment 173. The pharmaceutical composition of any one of embodiments 124-170, wherein two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

Embodiment 174. The pharmaceutical composition of any one of embodiments 124-170, wherein two vicinal K groups are taken together with the atoms to which they are attached to form a five-membered carbocyclic or heterocyclic ring, wherein the five-membered carbocyclic or heterocyclic ring are each optionally substituted with one or two substituents selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

Embodiment 175. The pharmaceutical composition of any one of embodiments 124-167, wherein ring C is selected from the group consisting of

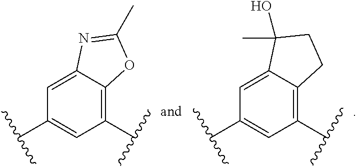 [SMILES: *c1cc(*)c2oc(C)nc2c1 ; *c1cc(*)c2c(c1)C(C)(O)CC2]

Embodiment 176. The pharmaceutical composition of any one of embodiments 124-175, wherein one of R⁷ and R⁸ is C₁-C₈ alkyl.

Embodiment 177. The pharmaceutical composition of any one of embodiments 124-175, wherein R⁷ is methyl.

Embodiment 178. The pharmaceutical composition of any one of embodiments 124-175, wherein R⁸ is methyl.

Embodiment 179. The pharmaceutical composition of any one of embodiments 124-175, wherein one of R⁷ and R⁸ is H, F, CF₃ or —CH₂OCH₃.

Embodiment 180. The pharmaceutical composition of any one of embodiments 124-175, wherein one of R⁷ and R⁸ is methyl and the other is H or F.

Embodiment 181. The pharmaceutical composition of any one of embodiments 124-175, wherein R⁷ and R⁸ together with the carbon to which they are attached form a C₃-C₈ cycloalkyl ring or three- to six-membered heterocyclyl ring, wherein the C₃-C₈ cycloalkyl ring or the three- to six-membered heterocyclyl ring is optionally substituted with F, Cl, Br, I, —OH, C₁-C₄ alkyl, or —O—C₁-C₄ alkyl.

Embodiment 182. The pharmaceutical composition of any one of embodiments 124-175, wherein R⁷ and R⁸ together with the carbon to which they are attached form a cyclopropyl or oxetanyl ring, wherein the cyclopropyl or oxetanyl ring is optionally substituted with F, Cl, Br, I, —OH, C₁-C₄ alkyl, or —O—C₁-C₄ alkyl.

Embodiment 183. The pharmaceutical composition of any one of embodiments 124-182, wherein Y is C(R⁹)(R¹⁰), wherein R⁹ is selected from the group consisting of H, F, Cl, Br, I, and C₁-C₈ alkyl, and R¹⁰ is selected from the group consisting of H, F, Cl, Br, I, and C₁-C₈ alkyl.

Embodiment 184. The pharmaceutical composition of embodiment 164, wherein Y is CH₂.

Embodiment 185. The pharmaceutical composition of any one of embodiments 124-182, wherein Y is S.

Embodiment 186. The pharmaceutical composition of any one of embodiments 124-175, wherein R⁹ is taken together with R⁸ to form a three- to six-membered cycloalkyl, heterocyclyl, aryl, or heteroaryl ring, each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

Embodiment 187. The pharmaceutical composition of any one of embodiments 124-175, wherein R⁹ is taken together with R⁸ to form a three- to six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

Embodiment 188. The pharmaceutical composition of any one of embodiments 124-175, wherein R⁹ is taken together with R⁸ to form a cyclopropyl, cyclobutyl, cyclopentyl, cyclohexyl, aziridinyl, oxiranyl, thiiranyl, azetidinyl, oxetanyl, thietanyl, pyrrolidinyl, pyrazolidinyl, imidazolidinyl, tetrahydrofuranyl, 1,3-dioxolanyl, tetrahydrothiophenyl, oxathiolanyl, sulfolanyl, piperidinyl, piperazinyl, tetrahydropyranyl, dioxanyl, thianyl, dithianyl, trithianyl, morpholinyl, or thiomorpholinyl ring.

Embodiment 189. The pharmaceutical composition of any one of embodiments 124-184 or 186-187, wherein Y is C(R⁹)(R¹⁰), and the absolute configuration of the carbon atom to which R⁷ and R⁸ are attached is (R).

Embodiment 190. The pharmaceutical composition of any one of embodiments 124-184 or 186-187, wherein Y is C(R⁹)(R¹⁰), and the absolute configuration of the carbon atom to which R⁷ and R⁸ are attached is (S).

Embodiment 191. The pharmaceutical composition of any one of embodiments 124-182 or 185, wherein Y is S, and the absolute configuration of the carbon atom to which R⁷ and R⁸ are attached is (S).

Embodiment 192. The pharmaceutical composition of any one of embodiments 124-182 or 185, wherein Y is S, and the absolute configuration of the carbon atom to which R⁷ and R⁸ are attached is (R).

Embodiment 193. The pharmaceutical composition of embodiment 124, wherein [symbol] is a double bond, Y is C(R⁹); and Z is C(R⁸), wherein R is taken together with R to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl.

Embodiment 194. The pharmaceutical composition of any one of embodiments 124-193, wherein Ring B is a five-membered heteroaryl ring containing at least one N, O, or S ring atom, wherein Ring B is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 195. The pharmaceutical composition of any one of embodiments 124-193, wherein Ring B is a five-membered heteroaryl ring containing at least one N, O, or S ring atom, wherein Ring B is optionally substituted with one, two, or three substituents independently selected from the group consisting of F, Cl, Br, and I.

Embodiment 196. The pharmaceutical composition of any one of embodiments 124-193, wherein Ring B is a five-membered heteroaryl ring containing at least one N, O, or S ring atom, wherein Ring B is optionally substituted with one, two, or three substituents independently selected from the group consisting of methyl, ethyl, cyclopropyl, and —CH₂OH.

Embodiment 197. The pharmaceutical composition of any one of embodiments 124-193, wherein Ring B is selected from the group consisting of pyrrole, imidazole, 1,2,4-triazole, 1,2,3-triazole, pyrazole, tetrazole, oxadiazole, oxazole, and isoxazole, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—Cᵣ C₈ haloalkyl.

Embodiment 198. The pharmaceutical composition of any one of embodiments 124-193, wherein Ring B is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—Cᵣ C₈ haloalkyl.

Embodiment 199. The pharmaceutical composition of any one of embodiments 124-193, wherein Ring B is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with methyl, ethyl, cyclopropyl, or —CH₂OH.

Embodiment 200. The pharmaceutical composition of any one of embodiments 124-193, wherein Ring B is selected from the group consisting of

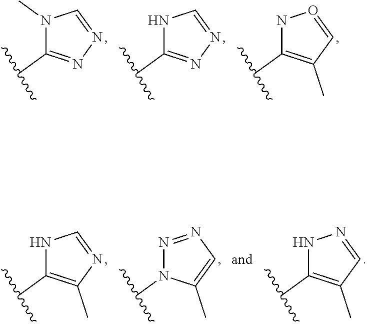

Embodiment 201. The pharmaceutical composition of any one of embodiments 124-193, wherein Ring B is selected from the group consisting of

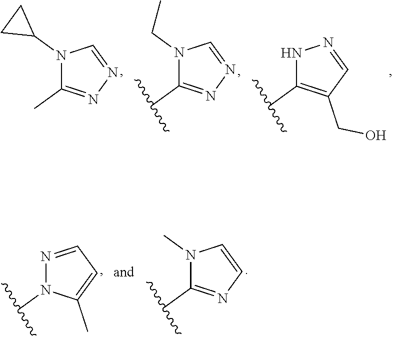

Embodiment 202. The pharmaceutical composition of any one of embodiments 124-193, wherein Ring B is selected from the group consisting of

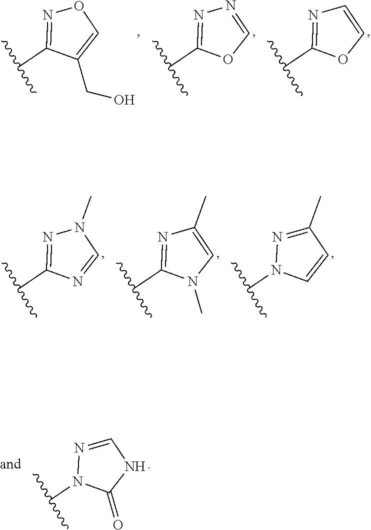

Embodiment 203. The pharmaceutical composition of any one of embodiments 124-193, wherein Ring B is selected from the group consisting of

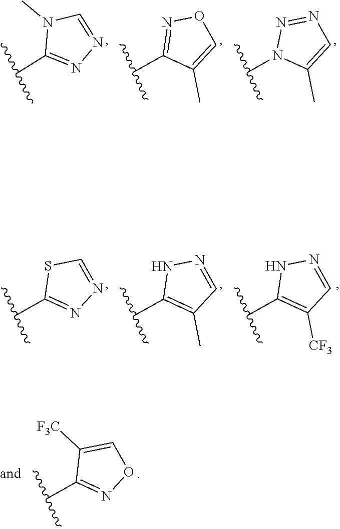

Embodiment 204. The pharmaceutical composition of any one of embodiments 124-193, wherein Ring B is 4-methyl-4H-1,2,4-triazol-3-yl.

Embodiment 205. The pharmaceutical composition of any one of embodiments 124-204, wherein the pharmaceutical composition is sterile.

Embodiment 206. The pharmaceutical composition of any one of embodiments 124-205, wherein the composition is suitable for intravenous, intraarterial, intramuscular, peritoneal, intrathecal, or subcutaneous injection.

Embodiment 207. The pharmaceutical composition of any one of embodiments 124-206, wherein the pharmaceutically acceptable excipient is selected from the group consisting of water, saline, Ringer's solution, or isotonic sodium chloride solution.

Embodiment 208. The pharmaceutical composition of any one of embodiments 124-207, with the proviso that the compounds of Formula (III-A) or Formula (III) excludes compounds of Table IX, tautomers of the compounds of Table IX, or salts of the compounds or tautomers.

Embodiment 209. The pharmaceutical composition of embodiment 124 wherein the compound of Formula (III-A) or Formula (III) is selected from the group consisting of the compounds of Table 1, including “a” and “b” variants of the compounds, tautomers thereof, and salts of the compounds or tautomers.

Embodiment 210. The pharmaceutical composition of embodiment 127, wherein the compound is selected from Compounds 1-7, 26-29, 31-47, 49-61, 64-77, 79-82, 84-85, 87-94, 95, 95a, 95b, 102a-106, 110, 113-114, 116-119, 122-127, 130a, 133, 138-161, 170, 181, 183a-195, 197-208, 212-254, 284-288, or 302 of Table 1, including “a” and “b” variants of the compounds, tautomers thereof, and pharmaceutically acceptable salts of the compounds or tautomers.

Embodiment 211. The pharmaceutical composition of embodiment 124, wherein the compound of Formula (III-A) or Formula (III) is selected from the group consisting of the compounds 8a, 57a, 140, 255a, 183a, 282, and 187, tautomers thereof, and pharmaceutically acceptable salts of the compounds or tautomers.

Embodiment 212. The pharmaceutical composition of embodiment 124, wherein the compound of Formula (III-A) or Formula (III) is selected from the group consisting of the compounds 8a, 57a, 140, 183a, and 187, tautomers thereof, and pharmaceutically acceptable salts of the compounds or tautomers.

Embodiment 213. The pharmaceutical composition of embodiment 124, wherein the compound of Formula (III-A) or Formula (III) is selected from the group consisting of the compounds 255a and 282, tautomers thereof, and pharmaceutically acceptable salts of the compounds or tautomers.

Embodiment 214. The pharmaceutical composition of embodiment 124, wherein the compound of Formula (III-A) or Formula (III) is selected from the group consisting of

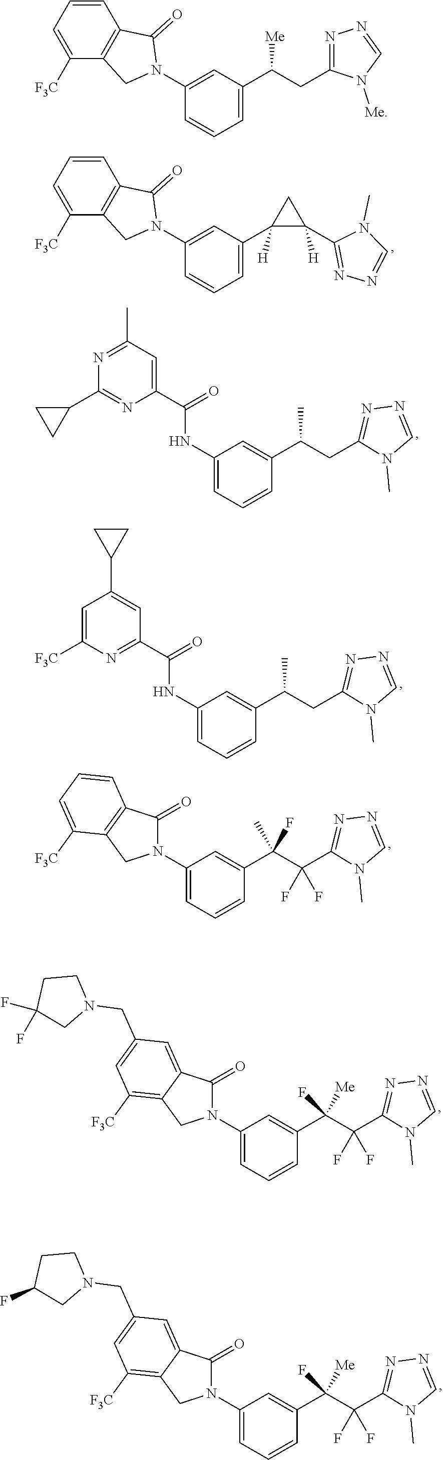

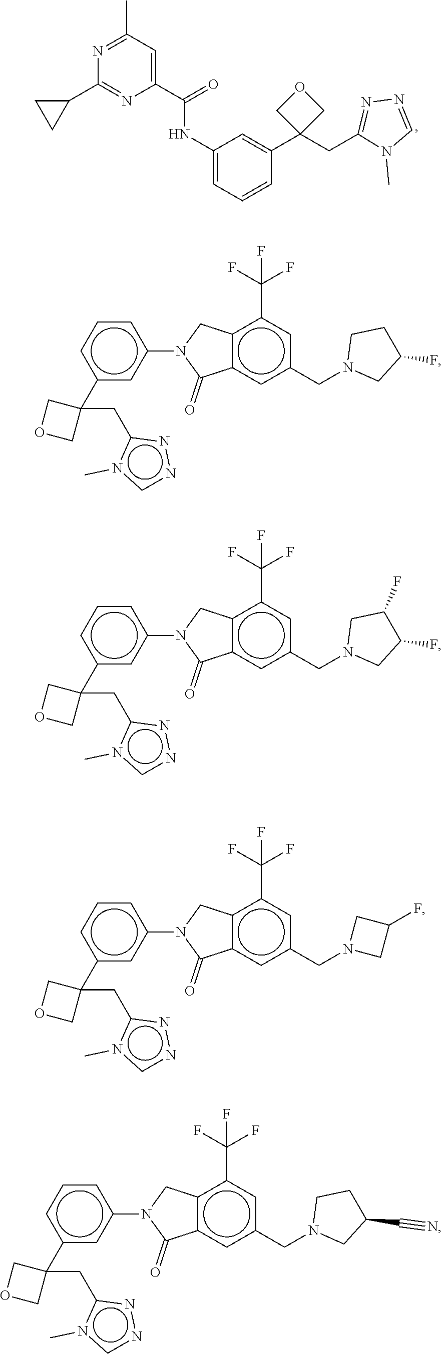

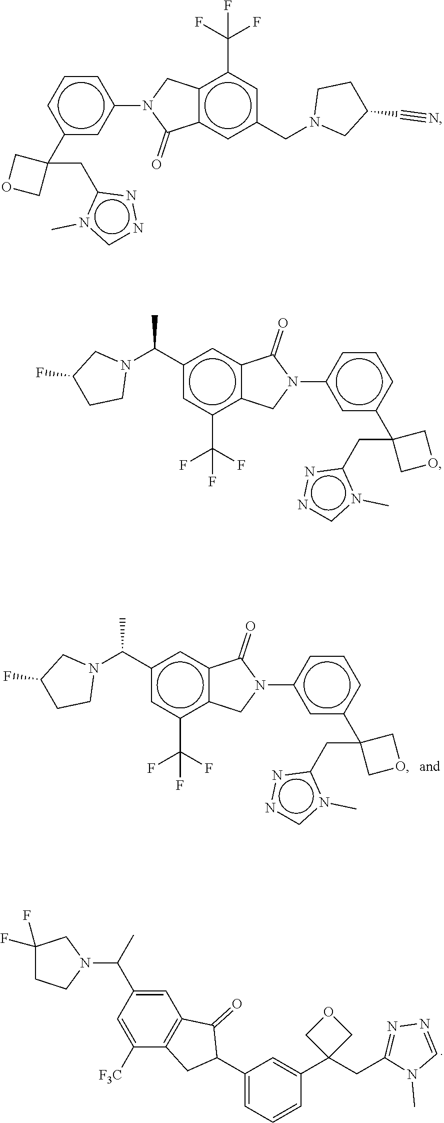

Embodiment 215. A compound of Formula (IV):

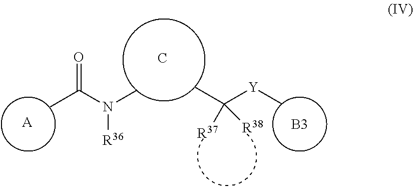

or a tautomer thereof, or a pharmaceutically acceptable salt of any of the foregoing, wherein Ring A,

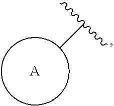

is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;

wherein Ring A is attached to the adjacent carbonyl carbon via a carbon atom of Ring A;

each R^{A} is independently selected from the group consisting of:

H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a —C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R³⁶ is H, or

R³⁶ and the amide group to which R³⁶ is connected attach to Ring A to form a five-membered lactam ring fused to Ring A, such that the fragment

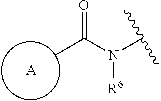

is

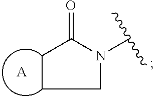

Ring C,

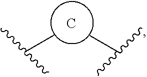

is selected from the group consisting of:

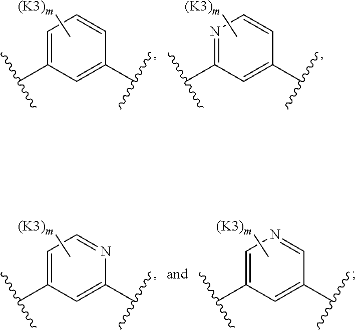

each K3 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —C₁-C₈ alkyl, —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m is 0, 1, or 2;

R³⁷ and R³⁸ together with the carbon to which they are attached form a C₃-C₈ cycloalkyl ring or three- to six-membered heterocyclyl ring as indicated by the dashed curve [symbol] , wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

Y is C(R³⁹)(R⁴⁰) or S;

R³⁹ and R⁴⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; or

R³⁹ and R⁴⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

and

Ring B3,

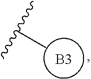

is a five-membered heteroaryl ring containing at least one N, O, or S ring atom, wherein Ring B3 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 216. The compound of embodiment 215, wherein

Ring A,

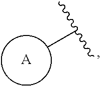

is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;

wherein Ring A is attached to the adjacent carbonyl carbon via a carbon atom of Ring A;

each R^{A} is independently selected from the group consisting of:

H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,

—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,

—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,

a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring, —(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

—S(═O)₂—C₁-C₈ alkyl,

—SF₅, and

—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;

R³⁶ is H, or

R³⁶ and the amide group to which R³⁶ is connected attach to Ring A to form a five-membered lactam ring fused to Ring A, such that the fragment

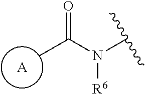

is

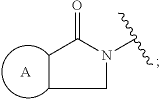

Ring C,

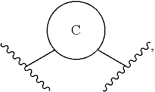

is selected from the group consisting of:

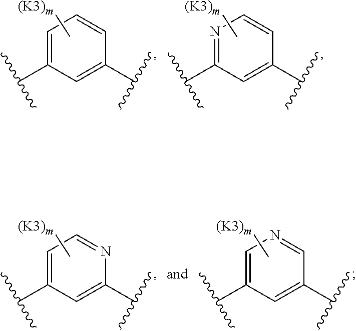

each K3 is independently selected from the group consisting of:

F, Cl, Br, I, —CN, —OH,

C₁-C₈ alkyl optionally substituted with —OH or —O—C₁-C₈ alkyl,

C₃-C₈ cycloalkyl optionally substituted with —OH or —O—C₁-C₈ alkyl,

—O—C₁-C₈ alkyl optionally substituted with —OH,

—O—C₁-C₈ haloalkyl,

—O-(three- to six-membered heterocyclic ring) optionally substituted with C₁-C₈ alkyl,

a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and

—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, and four- to eight-membered heterocyclyl optionally substituted with —OH or C₁-C₄ alkyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;

or two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;

m is 0, 1, or 2;

R³⁷ and R³⁸ together with the carbon to which they are attached form a C₃-C₈ cycloalkyl ring or three- to six-membered heterocyclyl ring as indicated by the dashed curve [symbol] , wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;

Y is C(R³⁹)(R⁴⁰) or S;

R³⁹ and R⁴⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; or

R³⁹ and R⁴⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH; and

Ring B3,

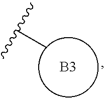

is a five-membered heteroaryl ring containing at least one N, O, or S ring atom, wherein Ring B3 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 217. The compound or tautomer thereof, or pharmaceutically acceptable salt of the compound or tautomer, of embodiment 215 or embodiment 216, wherein R³⁷ and R³⁸ together with the carbon to which they are attached combine to form an oxetane ring.

Embodiment 218. The compound or tautomer thereof, or pharmaceutically acceptable salt of the compound or tautomer, of embodiment 215 or embodiment 216, wherein R³⁷ and R³⁸ together with the carbon to which they are attached combine to form a cyclobutyl ring.

Embodiment 219. The compound or tautomer thereof, or pharmaceutically acceptable salt of the compound or tautomer, of embodiment 217, wherein the compound is of Formula (IV-ox):

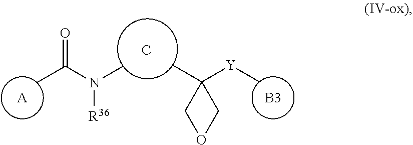

or a tautomer thereof, or a pharmaceutically acceptable salt of any of the foregoing. Embodiment 220. The compound or tautomer thereof, or pharmaceutically acceptable salt of the compound or tautomer, of any one of embodiments 215-219, wherein Ring B3 is selected from the group consisting of pyrrole, imidazole, 1,2,4-triazole, 1,2,3-triazole, pyrazole, tetrazole, oxadiazole, oxazole, and isoxazole, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

Embodiment 221. The compound or tautomer thereof, or pharmaceutically acceptable salt of the compound or tautomer, of any one of embodiments 215-219, wherein Ring B3 is 4-methyl-4H-1,2,4-triazol-3-yl.

Embodiment 222. The compound or tautomer thereof, or pharmaceutically acceptable salt of the compound or tautomer, of any one of embodiments 215-221, wherein Y is C(R³⁹)(R⁴⁰).

Embodiment 223. The compound or tautomer thereof, or pharmaceutically acceptable salt of the compound or tautomer, of embodiment 222, wherein R³⁹ is H and R⁴⁰ is H.

Embodiment 224. The compound or tautomer thereof, or pharmaceutically acceptable salt of the compound or tautomer, of embodiment 222, wherein at least one of R³⁹ and R⁴⁰ is F.

Embodiment 225. The compound or tautomer thereof, or pharmaceutically acceptable salt of the compound or tautomer, of embodiment 222, wherein R³⁹ is H and R⁴⁰ is F.

Embodiment 226. The compound or tautomer thereof, or pharmaceutically acceptable salt of the compound or tautomer, of embodiment 222, wherein R³⁹ is F and R⁴⁰ is F.

Embodiment 227. The compound or tautomer thereof, or pharmaceutically acceptable salt of the compound or tautomer, of embodiment 222, wherein R³⁹ and R⁴⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl.

Embodiment 228. The compound or tautomer thereof, or pharmaceutically acceptable salt of the compound or tautomer, of embodiment 227, wherein R³⁹ and R⁴⁰ together with the carbon to which they are attached form a C₃ cycloalkyl ring optionally substituted with F, Cl, Br, I, —OH, C₁-C₄ alkyl, or —O—C₁-C₄ alkyl.

Embodiment 229. The compound or tautomer thereof, or pharmaceutically acceptable salt of the compound or tautomer, of any one of embodiments 215-228, wherein Ring C is

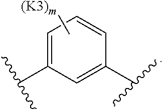

Embodiment 230. The compound or tautomer thereof, or pharmaceutically acceptable salt of the compound or tautomer, of embodiment 229, wherein m is 0.

Embodiment 231. The compound or tautomer thereof, or a pharmaceutically acceptable salt of the compound or tautomer, of any one of embodiments 215-230, or the composition of any one of embodiments 124-214, wherein Ring A is phenyl, pyridyl, or pyrimidyl.

Embodiment 232. The compound or tautomer thereof, or pharmaceutically acceptable salt of the compound or tautomer, of any one of embodiments 215-231, or the composition of any one of embodiments 124-214, wherein Ring A is pyrimidyl. Embodiment 233. The compound or tautomer thereof, or a pharmaceutically acceptable salt of the compound or tautomer, of any one of embodiments 215-232, or the composition of any one of embodiments 124-214, wherein the Ring A is substituted with one or more F, —CF₃, or cyclopropyl groups.

Embodiment 234. The compound or tautomer thereof, or pharmaceutically acceptable salt of the compound or tautomer, of any one of embodiments 215-230, or the composition of any one of embodiments 124-214, wherein Ring A is phenyl, pyridyl, or pyrimidyl and is substituted with one, two, or three C₁-C₈ alkyl, C₁-C₈ haloalkyl, C₃-C₈ cycloalkyl, CH₃, CF₃, or cyclopropyl groups.

Embodiment 235. The compound or tautomer thereof, or pharmaceutically acceptable salt of the compound or tautomer, of any one of embodiments 215-230, or the composition of any one of embodiments 124-214, wherein Ring A is phenyl, pyridyl, or pyrimidyl and is substituted with one or more F, —CF₃, or cyclopropyl groups.

Embodiment 236. The compound or tautomer thereof, or pharmaceutically acceptable salt of the compound or tautomer, of embodiment 215 or embodiment 216, wherein the compound is selected from the group consisting of

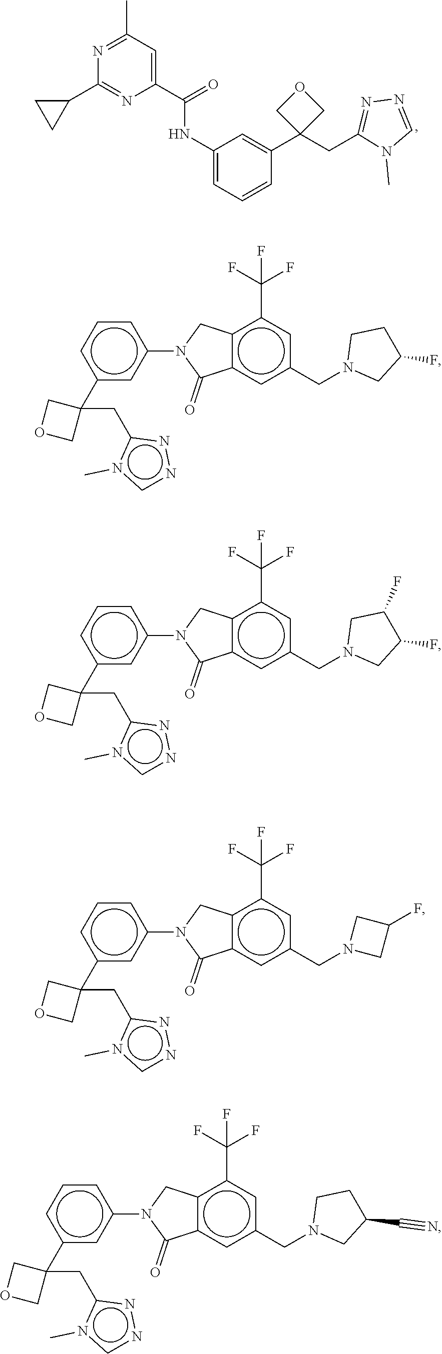

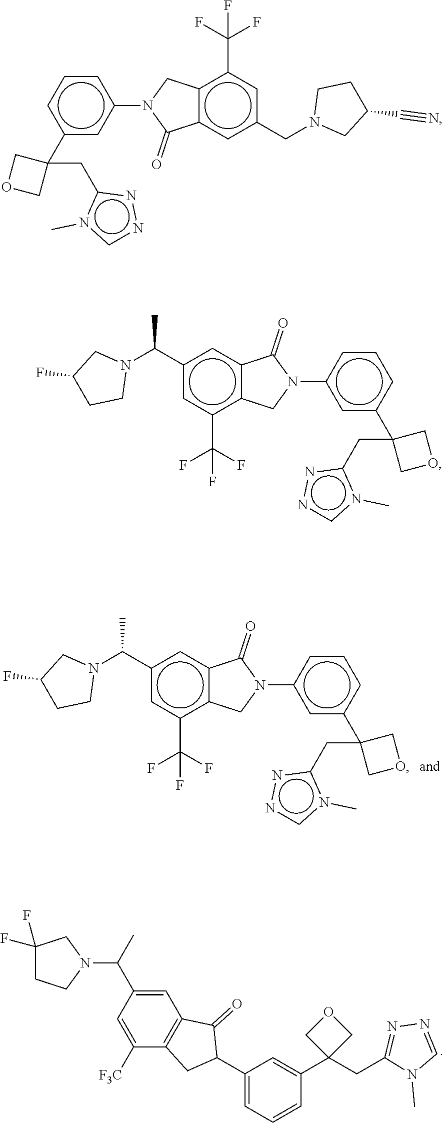

Embodiment 237. The compound or tautomer thereof, or pharmaceutically acceptable salt of the compound or tautomer, of any one of embodiments 1-123 and 215-236, wherein said compound, tautomer, or salt has a Cbl-b inhibition IC₅₀ of about 1 micromolar or less; or a Cbl-b inhibition IC₅₀ of about 300 nanomolar or less; or a compound or tautomer thereof, or pharmaceutically acceptable salt of the compound or tautomer, of Formula III-A or Formula III as in any one of claims 124-214, wherein said compound, tautomer, or salt has a Cbl-b inhibition IC₅₀ of about 1 micromolar or less; or a Cbl-b inhibition IC₅₀ of about 300 nanomolar or less.

Embodiment 238. The compound or tautomer thereof, or pharmaceutically acceptable salt of the compound or tautomer, of any one of embodiments 1-123 and 215-236, wherein said compound, tautomer, or salt has a Cbl-b binding K_{D} of about 1 micromolar or less; or a Cbl-b binding K_{D} of about 300 nanomolar or less; or a compound or tautomer thereof, or pharmaceutically acceptable salt of the compound or tautomer, of Formula III-A or Formula III as in any one of claims 124-214, wherein said compound, tautomer, or salt has a Cbl-b binding K_{D} of about 1 micromolar or less; or a Cbl-b binding K_{D} of about 300 nanomolar or less.

Embodiment 239. A method of modulating activity of an immune cell, the method comprising contacting the immune cell with an effective amount of a Cbl-b inhibitor to modulate activity of the immune cell, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 240. The method of embodiment 239, wherein the immune cell comprises a T-cell, a B cell, or a natural killer (NK) cell.

Embodiment 241. The method of embodiment 239 or embodiment 240, wherein the immune cell is a tumor-infiltrating lymphocyte (TIE) isolated from a tumor of a mammalian subject with cancer before the immune cell is contacted with the Cbl-b inhibitor.

Embodiment 242. The method of any one of embodiments 239-241, further comprising isolating the immune cell from a tumor of a mammalian subject with cancer before the immune cell is contacted with the Cbl-b inhibitor.

Embodiment 243. The method of any one of embodiments 239-242, wherein the immune cell comprises a T-cell, and wherein modulating activity of the T-cell comprises one or more of increased T-cell activation, increased T-cell proliferation, decreased T-cell exhaustion, and decreased T-cell tolerance.

Embodiment 244. The method of embodiment 243, wherein increased T-cell activation comprises increased production of a cytokine.

Embodiment 245. The method of embodiment 244, wherein the cytokine comprises one or more selected from the group consisting of IL-2, IFN-γ, TNFα, and GM-CSF.

Embodiment 246. The method of any one of embodiments 243-244, wherein increased T-cell activation comprises increased cell surface expression of one or more T-cell activation markers.

Embodiment 247. The method of embodiment 246, wherein the T-cell activation markers comprise one or more selected from the group consisting of CD25, CD69, and CTLA4.

Embodiment 248. The method of any one of embodiments 243-247, wherein the T-cell has been or is in contact with an anti-CD3 antibody alone or in combination with an anti-CD28 antibody.

Embodiment 249. The method of any one of embodiments 243-247, further comprising culturing the immune cell with IL-2 alone or in combination with an anti-CD3 antibody and/or an anti-CD28 antibody.

Embodiment 250. The method of any one of embodiments 239-242, wherein the immune cell comprises a NK cell, and wherein modulating activity of an NK cell comprises increased NK cell activation.

Embodiment 251. The method of embodiment 250, wherein increased NK cell activation comprises increased production of a cytokine.

Embodiment 252. The method of embodiment 251, wherein the cytokine comprises one or more selected from the group consisting of IFN-γ, TNFα, and MIP1β.

Embodiment 253. The method of any one of embodiments 239-252, wherein the immune cell comprises a B cell, and wherein modulating activity of a B cell comprises increased B cell activation, optionally wherein increased B cell activation comprises increased expression of CD69.

Embodiment 254. The method of any one of embodiments 239-253, wherein the immune cell is a human immune cell.

Embodiment 255. The method of any one of embodiments 239-254, wherein the immune cell comprises a recombinant chimeric receptor.

Embodiment 256. The method of embodiment 255, wherein the recombinant chimeric receptor is a chimeric antigen receptor.

Embodiment 257. A method of producing a modified immune cell, comprising culturing a cell population containing an immune cell in the presence of an effective amount of a Cbl-b inhibitor to modulate activity of the immune cell, thereby producing the modified immune cell, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 258. The method of embodiment 257, further comprising culturing the immune cell with an anti-CD3 antibody alone or in combination with an anti-CD28 antibody.

Embodiment 259. The method of embodiment 257, further comprising culturing of the immune cell with IF-2 alone or in combination with an anti-CD3 antibody and/or an anti-CD28 antibody

Embodiment 260. The method of any one of embodiments 257-259, further comprising recovering the modified immune cell.

Embodiment 261. The method of any one of embodiments 257-260, wherein the immune cell is a tumor-infiltrating lymphocyte (TIE).

Embodiment 262. The method of any one of embodiments 257-260, wherein the immune cell is a cell selected from the group consisting of: a hematopoietic cell, a multipotent stem cell, a myeloid progenitor cell, a lymphoid progenitor cell, a T-cell, a B cell, and a NK cell.

Embodiment 263. The method of any one of embodiments 257-260, wherein the modified immune cell is a cell selected from the group consisting of: a hematopoietic cell, a multipotent stem cell, a myeloid progenitor cell, a lymphoid progenitor cell, a T-cell, a B cell, and a NK cell.

Embodiment 264. The method of any one of embodiments 257-263, wherein the immune cell is from an individual.

Embodiment 265. The method of any one of embodiments 257-264, wherein the immune cell is a human immune cell.

Embodiment 266. The method of any one of embodiments 257-265, wherein the immune cell or modified immune cell comprises a recombinant chimeric receptor.

Embodiment 267. The method of embodiment 266, wherein the recombinant chimeric receptor is a chimeric antigen receptor.

Embodiment 268. A modified immune cell produced by the method of any one of embodiments 257-267.

Embodiment 269. A modified immune cell comprising a Cbl-b inhibitor, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 270. An isolated modified immune cell, wherein the immune cell has been contacted or is in contact with a Cbl-b inhibitor, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 271. The modified immune cell of embodiment 270, wherein the modified immune cell is a T-cell, a B cell or a NK cell.

Embodiment 272. The modified immune cell of embodiment 270 or embodiment 271, wherein the immune cell is a tumor-infiltrating lymphocyte (TIE) isolated from a tumor of a mammalian subject with cancer before the immune cell is contacted with the Cbl-b inhibitor

Embodiment 273. The modified immune cell of any one of embodiments 270-272, wherein the modified immune cell is a T-cell, and wherein the T-cell exhibits one or more of increased T-cell activation, increased T-cell proliferation, decreased T-cell exhaustion, and decreased T-cell tolerance.

Embodiment 274. The modified immune cell of embodiment 273, wherein increased T-cell activation comprises increased production of a cytokine.

Embodiment 275. The modified immune cell of embodiment 274, wherein the cytokine comprises one or more selected from the group consisting of IF-2, IFN-γ, TNFα, and GM-CSF.

Embodiment 276. The modified immune cell of any one of embodiments 273-275, wherein increased T-cell activation comprises increased cell surface expression of one or more T-cell activation markers.

Embodiment 277. The modified immune cell of embodiment 276, wherein the T-cell activation markers comprise one or more selected from the group consisting of CD25, CD69, and CTLA4.

Embodiment 278. The modified immune cell of any one of embodiments 273-277, wherein the T-cell has been or is in contact with an anti-CD3 antibody alone or in combination with an anti-CD28 antibody.

Embodiment 279. The modified immune cell of any one of embodiments 273-277, wherein the T-cell has been or is in contact with IF-2 alone or in combination with an anti-CD3 antibody and/or an anti-CD28 antibody.

Embodiment 280. The modified immune cell of any one of embodiments 273-272, wherein the modified immune cell is a NK cell, and wherein the NK cell exhibits increased NK cell activation.

Embodiment 281. The modified immune cell of embodiment 280, wherein increased NK cell activation comprises increased production of a cytokine.

Embodiment 282. The modified immune cell of embodiment 281, wherein the cytokine comprises one or more selected from the group consisting of IFN-γ, TNFα, and MIP1β.

Embodiment 283. The modified immune cell of any one of embodiments 270-272, wherein the modified immune cell is a B cell, and wherein the B cell exhibits increased B cell activation, optionally wherein increased B cell activation comprises increased expression of CD69.

Embodiment 284. The modified immune cell of any one of embodiments 270-283, wherein the modified immune cell is a human immune cell.

Embodiment 285. The modified immune cell of any one of embodiments 270-284, wherein the modified immune cell comprises a recombinant chimeric receptor.

Embodiment 286. The modified immune cell of embodiment 285, wherein the recombinant chimeric receptor is a chimeric antigen receptor.

Embodiment 287. A composition comprising a cell population containing the modified immune cell of any one of embodiments 268-286.

Embodiment 288. The composition of embodiment 287, further comprising a pharmaceutically acceptable excipient.

Embodiment 289. The composition of embodiment 287, wherein the composition is in a culture vessel.

Embodiment 290. The composition of embodiment 289, wherein the culture vessel is a tube, a dish, a bag, a multiwell plate or a flask.

Embodiment 291. The composition of embodiment 287 or embodiment 288, wherein the composition is in a suitable container.

Embodiment 292. The composition of embodiment 291, wherein the suitable container is a bottle, a vial, a syringe, an intravenous bag or a tube.

Embodiment 293. A method of modulating the immune response, the method comprising administering an effective amount of the modified immune cell of any one of embodiments 268-286 or an effective amount of the composition of any one of embodiments 287-292 to an individual in need thereof.

Embodiment 294. The method of embodiment 293, wherein the individual has a cancer.

Embodiment 295. A method of treating a cancer responsive to inhibition of Cbl-b activity, the method comprising administering an effective amount of the modified immune cell of any one of embodiments 268-286 or an effective amount of the composition of any one of embodiments 287-292 to an individual having the cancer responsive to inhibition of Cbl-b activity.

Embodiment 296. The method of embodiment 294 or 295 wherein the cancer is a hematologic cancer.

Embodiment 297. The method of embodiment 296, wherein the hematologic cancer is a lymphoma, a leukemia, or a myeloma.

Embodiment 298. The method of embodiment 294 or 295 wherein the cancer is a non-hematologic cancer.

Embodiment 299. The method of embodiment 298, wherein the non-hematologic cancer is a sarcoma or a carcinoma.

Embodiment 300. A method of inhibiting abnormal cell proliferation, the method comprising administering an effective amount of the modified immune cell of any one of embodiments 268-286 or an effective amount of the composition of any one of embodiments 287-292 to an individual in need thereof.

Embodiment 301. The method of embodiment 300, wherein the abnormal cell proliferation is hyperplasia or cancer cell proliferation.

Embodiment 302. The method of embodiment 301, wherein the cancer cell is from a hematologic cancer.

Embodiment 303. The method of embodiment 302, wherein the hematologic cancer is a lymphoma, a leukemia, or a myeloma.

Embodiment 304. The method of embodiment 301, wherein the cancer cells is from a non-hematologic cancer.

Embodiment 305. The method of embodiment 304, wherein the non-hematologic cancer is a sarcoma or a carcinoma.

Embodiment 306. A method of modulating the immune response, the method comprising administering an effective amount of a Cbl-b inhibitor to an individual to modulate the immune response in the individual, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 307. A method of inhibiting Cbl-b activity, the method comprising administering an effective amount of a Cbl-b inhibitor to an individual to inhibit Cbl-b activity in the individual, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 308. A method of treating a cancer responsive to inhibition of Cbl-b activity, the method comprising administering an effective amount of a Cbl-b inhibitor to an individual to treat the cancer responsive to inhibition of Cbl-b activity, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 309. The method of embodiment 308, wherein the cancer is a hematologic cancer.

Embodiment 310. The method of embodiment 298, wherein the hematologic cancer is a lymphoma, a leukemia, or a myeloma.

Embodiment 311. The method of embodiment 308, wherein the cancer is a non-hematologic cancer, is a sarcoma or a carcinoma.

Embodiment 312. The method of embodiment 311, wherein the non-hematologic cancer is a sarcoma or a carcinoma.

Embodiment 313. The method of any one of embodiments 308-312, further comprising administering an effective amount of the modified immune cell of any one of embodiments 268-286 or an effective amount of the composition of any one of embodiments 287-292 to the individual to treat the cancer.

Embodiment 314. A method of inhibiting abnormal cell proliferation, the method comprising administering an effective amount of a Cbl-b inhibitor to an individual to inhibit abnormal cell proliferation in the individual, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 315. The method of embodiment 314, wherein the abnormal cell proliferation is hyperplasia or cancer cell proliferation.

Embodiment 316. The method of embodiment 315, wherein the cancer cell is from a hematologic cancer.

Embodiment 317. The method of embodiment 316, wherein the hematologic cancer is a lymphoma, a leukemia, or a myeloma.

Embodiment 318. The method of embodiment 315, wherein the cancer cell is from a non-hematologic cancer.

Embodiment 319. The method of embodiment 318, wherein the non-hematologic cancer is a sarcoma or a carcinoma.

Embodiment 320. The method of any one of embodiments 306-319, wherein the individual has one or more of increased T-cell activation, increased T-cell proliferation, decreased T-cell exhaustion, and decreased T-cell tolerance after administration of the Cbl-b inhibitor.

Embodiment 321. The method of embodiment 320, wherein increased T-cell activation comprises increased production of a cytokine.

Embodiment 322. The method of embodiment 321, wherein the cytokine comprises one or more selected from the group consisting of IL-2, IFN-γ, TNFα, and GM-CSF.

Embodiment 323. The method of any one of embodiments 320-322, wherein increased T-cell activation comprises increased cell surface expression of one or more T-cell activation markers.

Embodiment 324. The method of embodiment 323, wherein the T-cell activation markers comprise one or more selected from the group consisting of CD25, CD69, and CTLA4.

Embodiment 325. The method of any one of embodiments 306-324, wherein the individual has increased NK cell activation after administration of the Cbl-b inhibitor.

Embodiment 326. The method of embodiment 325, wherein increased NK cell activation comprises increased production of a cytokine.

Embodiment 327. The method of embodiment 326, wherein the cytokine comprises one or more selected from the group consisting of IFN-γ, TNFα, and MIP1β.

Embodiment 328. The method of any one of embodiments 306-327, wherein the individual has increased B cell activation after administration of the Cbl-b inhibitor, optionally wherein increased B cell activation comprises increased expression of CD69.

Embodiment 329. A cell culture composition comprising a cell population containing an immune cell and a Cbl-b inhibitor, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 330. The cell culture composition of embodiment 329, wherein the immune cell is a cell selected from the group consisting of: a hematopoietic cell, a multipotent stem cell, a myeloid progenitor cell, a lymphoid progenitor cell, a T-cell, a B cell, and a NK cell.

Embodiment 331. The cell culture composition of embodiment 329 or 330, further comprising an anti-CD3 antibody alone or in combination with an anti-CD28 antibody.

Embodiment 332. The cell culture composition of any one of embodiments 329-331, wherein the immune cell is an engineered immune cell comprising a recombinant chimeric receptor.

Embodiment 333. The cell culture composition of embodiment 332, wherein the recombinant chimeric receptor is a chimeric antigen receptor.

Embodiment 334. A pharmaceutical composition comprising a Cbl-b inhibitor and one or both of an adjuvant and an antigen, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 335. The pharmaceutical composition of embodiment 334, wherein the antigen is a cancer antigen.

Embodiment 336. An article of manufacture comprising the modified immune cell of any one of embodiments 268-286, the composition of any one of embodiments 287-292, the cell culture composition of any one of embodiments 329-333, or the pharmaceutical composition of any one of embodiments 124-214.

Embodiment 337. The article of manufacture of embodiment 336, wherein the modified immune cell or cell culture composition is in a tube, a dish, a bag, a multiwell plate or a flask.

Embodiment 338. The article of manufacture of embodiment 336, wherein the modified immune cell or pharmaceutical composition is in a bottle, a vial, a syringe, an intravenous bag or a tube.

Embodiment 339. A kit comprising the modified immune cell of any one of embodiments 268-286 or the composition of any one of embodiments 287-292.

Embodiment 340. The kit of embodiment 339, wherein the modified immune cell is in a tube, a dish, a bag, a multiwell plate, or a flask.

Embodiment 341. The kit of embodiment 339, wherein the modified immune cell is in a bottle, a vial, a syringe, an intravenous bag, or a tube.

Embodiment 342. The kit of any one of embodiments 339-341, wherein the kit comprises instructions for administering the modified immune cell or composition to an individual according to the method of any one of embodiments 293-305.

Embodiment 343. A kit comprising the pharmaceutical composition of any one of embodiments 124-214.

Embodiment 344. The kit of embodiment 343, wherein the kit comprises instructions for administering the pharmaceutical composition to an individual according to the method of any one of embodiments 306-308.

Embodiment 345. A kit comprising the cell culture composition of any one of embodiments 329-333.

Embodiment 346. The kit of embodiment 345, wherein the kit comprises instructions for producing a modified immune cell according to the method of any one of embodiments 257-267.

Embodiment 347. A method for treating or preventing a disease or condition associated with Cbl-b activity, the method comprising administering a Cbl-b inhibitor to an individual in need thereof, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 348. Use of a Cbl-b inhibitor in the manufacture of a medicament for treating or preventing a disease or condition associated with Cbl-b activity, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 349. Use of a Cbl-b inhibitor in the manufacture of a medicament for treating cancer, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 350. A Cbl-b inhibitor for use in treating or preventing a disease or condition associated with Cbl-b activity, wherein the Cbl-b inhibitor is a compound of Formula (I-A), Formula (I), Formula (II-A), Formula (II), Formula (III), Formula (III-A), or Formula (IV) of any one of embodiments 1-238.

Embodiment 351. A Cbl-b inhibitor for use in treating cancer, wherein the Cbl-b inhibitor is a compound of Formula (I-A), Formula (I), Formula (II-A), Formula (II), Formula (III), Formula (III-A), or Formula (IV) of any one of embodiments 1-238.

### EXAMPLES

Examples A-AH below describe synthesis of key intermediates used for the compounds disclosed in Examples 1-485. General procedures for synthesis, including General Procedure 1-G, General Work-Up Procedure, and Purification Procedures, are listed after Example AC, except for General Procedure 4 (see Example 98, step 3) and General Procedure 5 (see Example 138). Chromatography A refers to purification over silica gel, typically in pre-packed cartridges, eluting with mixtures of EtOAc in hexanes or petroleum ether; Chromatography B refers to elution with mixtures of MeOH in DCM; Chromatography C refers to use of C₁₋₈ reverse-phase silica gel, eluting with mixtures of acetonitrile in water; Chromatography D refers to elution with mixtures of ethanol, EtOAc and hexanes. Biological Examples follow Example 719. Compounds drawn without stereochemistry in Table 1 were tested as racemic mixtures in the Biological Examples. Abbreviations used in the Examples include the following: DAST: (Diethylamino)sulfur trifluoride; DCM: dichloromethane; EDC: N-Ethyl-N′-(3-dimethylaminopropyl)carbodiimide hydrochloride; EtOAc: ethyl acetate; HATU: 1-[bis(dimethylamino)methylene]-1H-1,2,3-triazolo[4,5-b]pyridinium 3-oxide hexafluorophosphate; THF: tetrahydrofuran; SPhos: 2-Dicyclohexylphosphino-2′,6′-dimethoxybiphenyl; XPhos: 2-Dicyclohexylphosphino-2′,4′,6′-triisopropylbiphenyl; and Xantphos: 4,5-Bis(diphenylphosphino)-9,9-dimethylxanthene; NBS, N-bromosuccinimide; NCS, N-chlorosuccinimide; BPO, benzoyl peroxide; TEA, triethylamine; DIPEA, N,N-Diisopropylethylamine; DMF, N,N-dimethylformamide.

### Example A

### 3-(1-(4-methyl-4H-1,2,4-triazol-3-ylthio)ethyl)aniline (A)

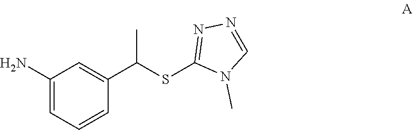 [SMILES: CC(Sc1nncn1C)c1cccc(N)c1]

Step 1: Synthesis of 4-methyl-3-(1-(3-nitrophenyl)ethylthio)-4H-1,2,4-triazole. A mixture of 1-(3-nitrophenyl)ethanol (10.0 g, 59.88 mmol), 4-methyl-4H-1,2,4-triazole-3-thiol (8.3 g, 71.86 mmol) and triphenylphosphine (31.0 g, 119.8 mmol) in THF (200 mL) was cooled 0° C. and diisopropyl diazene-1,2-dicarboxylate (24 g, 119.76 mmol) was added dropwise. The mixture was allowed to warm to about 25° C. for about 3 h. The mixture was quenched by the addition of water (150 mL) and following General Work-up Procedure 1, the resulting residue was purified by Chromatography B to afford the title compound (6.4 g, 40%) as a light yellow solid.

Step 2: Synthesis of 3-(1-(4-methyl-4H-1,2,4-triazol-3-ylthio)ethyl)aniline (A): To a stirred solution of 4-methyl-3-[[1-(3-nitrophenyl)ethyl]sulfanyl]-4H-1,2,4-triazole (6.4 g, 24.24 mmol) and ammonium chloride (7.8 g, 145.46 mmol) in ethanol (80 mL) and water (40 mL) was added iron powder (4.1 g, 72.72 mmol) in portions at about 25° C. The mixture was heated at about 80° C. for 5 h. The solids were filtered off and filtrate collected and concentrated to afford A (5.8 g, crude) as yellow oil, which used to next step without purification.

### (S)-3-(1-(4-methyl-4H-1,2,4-triazol-3-ylthio)ethyl)aniline (A-a)

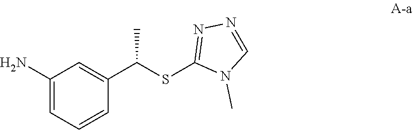 [SMILES: C[C@H](Sc1nncn1C)c1cccc(N)c1]

Step 1: Synthesis of (R)-1-(3-nitrophenyl)ethanol. To a solution of S-Me-CBS (12.1 mL, 12.1 mmol) in anhydrous toluene (300 mL) was added borane-N,N-diethylaniline complex (21.7 g, 133 mmol) at 30° C. under nitrogen atmosphere and stirred 20 min. And then a solution of l-(3-nitrophenyl)ethanone (20.0 g, 121 mmol) in toluene (200 mL) was added dropwise slowly over 5 h while maintaining the internal temperature at 30° C. The mixture was stirred for another 30 min and monitored by TLC. The reaction was quenched by the addition of a hydrochloric acid solution (4 N in methanol, 50 mL) and then diluted with water (150 mL). Following General Work-up Procedure 1, the resulting residue was triturated with 10% EtOAcEtOAc in petroleum ether to afford the title compound (15.0 g, 74%) as a light yellow solid. MS (ESI) calculated for (C₈H₉NO₃) [M+H]+, 168.1; found, 168.1.

Step 2: Synthesis of (S)-4-methyl-3-(1-(3-nitrophenyl)ethylthio)-4H-1,2,4-triazole. To a stirred solution of (R)-1-(3-nitrophenyl)ethanol (7.00 g, 41.9 mmol), 4-methyl-4H-1,2,4-triazole-3-thiol (5.78 g, 50.3 mmol) and triphenylphosphine (16.47 g, 62.9 mmol) in THF (100 mL) was added diisopropyl azodicarboxylate (12.70 g, 62.9 mmol) dropwise at 0° C. The mixture was warmed to rt and stirred for 1.5 h. The mixture was diluted with water (80 mL). followed by General Work-up Procedure 1 and the resulting residue was purified by Chromatography B to afford the title compound (8.90 g, 80%) as a colorless oil. MS (ESI) calculated for (C₁₁H₁₂N₄O₂S) [M+H]⁺, 265.1; found, 265.0.

Step 3: Synthesis of (S)-3-(1-(4-methyl-4H-1,2,4-triazol-3-ylthio)ethyl)aniline (A-a): To a solution of (S)-4-methyl-3-(1-(3-nitrophenyl)ethylthio)-4H-1,2,4-triazole (8.9 g, 33.7 mmol) in ethanol (150 mL) was added ammonium chloride (10.7 g, 202.2 mmol) and iron powder (5.7 g, 101.1 mmol). The mixture was stirred at 80° C. overnight and then was filtered through a Celite pad. The filtrate was concentrated to give a crude solid which was re-suspended in EtOAcEtOAc (100 mL) and methanol (5 mL). The insolubles were filtered off, and the filtrate was concentrated to give A-a (7.8 g, crude) as a yellow oil, which was used without purification. The analytical sample was obtained by Chromatography C.

### (R)-3-(1-((4-methyl-4H-1,2,4-triazol-3-yl)thio)ethyl)aniline (A-b)

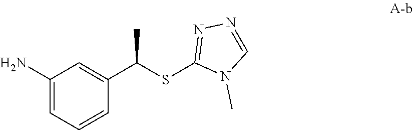 [SMILES: C[C@@H](Sc1nncn1C)c1cccc(N)c1]

Step 1: Synthesis of (S)-1-(3-nitrophenyl)ethanol. To a solution of (R)-Me-CBS (18.1 mL, 18.1 mmol) in anhydrous toluene (500 mL) was added borane-N,N-diethylaniline complex (32.6 g, 199 mmol) at 30° C. under nitrogen atmosphere and stirred 20 min. And then a solution of l-(3-nitrophenyl)ethanone (30.0 g, 182 mmol) in toluene (300 mL) was added dropwise over 5 h while maintaining the internal temperature at 30° C. The mixture was stirred for another 30 min and monitored by TLC. The reaction was quenched by the addition of hydrochloric acid solution (4 N in methanol, 50 mL) and then diluted with water (200 mL). After General Work-up Procedure 1, the resulting residue was purified by trituration with 10% EtOAcEtOAc in petroleum ether to afford the title compound (25.0 g, 83%) as a light yellow solid. MS (ESI) calculated for (C₈H₉NO₃) [M+H]⁺, 168.1; found, 168.1.

Step 2: Synthesis of (R)-4-methyl-3-(1-(3-nitrophenyl)ethylthio)-4H-1,2,4-triazole. To a stirred solution of (S)-1-(3-nitrophenyl)ethanol (25.0 g, 150 mmol), 4-methyl-4H-1,2,4-triazole-3-thiol (20.7 g, 180 mmol) and triphenylphosphine (47.3 g, 180 mmol) in THF (300 mL) was added diisopropyl azodicarboxylate (36.4 g, 180 mmol) dropwise at 0° C. The mixture was warmed to rt and stirred for 3 h. The mixture was quenched with water (200 mL). Following General Work-up Procedure 1, the resulting residue was purified by Chromatography B to afford the title compound (23.0 g, 58%) as a colorless oil. MS (ESI) calculated for (C₁₁H₁₂N₄O₂S) [M+H]⁺, 265.1; found, 265.0.

Step 3: Synthesis of (R)-3-(1-((4-methyl-4H-1,2,4-triazol-3-yl)thio)ethyl)aniline (A-b): To a solution of (R)-4-methyl-3-(1-(3-nitrophenyl)ethylthio)-4H-1,2,4-triazole (23.0 g, 86.9 mmol) in ethanol (2.5 L) was added ammonium chloride (27.6 g, 521.7 mmol) and iron powder (14.6 g, 260.8 mmol). The mixture was stirred at 80° C. for 5 h. And then was filtered through Celite pad. The filtrate was concentrated to give a crude solid which was re-suspended in EtOAc (200 mL) and methanol (10 mL). The insolubles were filtered off, and the filtrate was concentrated to give title compound (15.3 g, crude) as a yellow oil, which was used without purification. MS (ESI) calculated for (C₁₁H₁₄N₄S) [M+H]⁺, 235.1; found, 235.1.

### Example B

### 2-methyl-5-[1-[(4-methyl-4H-1,2,4-triazol-3-yl)sulfanyl]ethyl]-1,3-benzoxazol-7-amine (B-1)

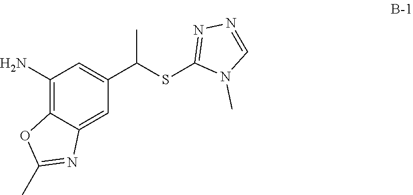 [SMILES: Cc1nc2cc(C(C)Sc3nncn3C)cc(N)c2o1]

Step 1: Synthesis of N-(5-bromo-2-hydroxyphenyl)acetamide. A mixture of 2-amino-4-bromophenol (35.0 g, 186 mmol) in MTBE (500 mL) was heated at reflux for 30 min under nitrogen. Then acetic anhydride (20.9 g, 205 mmol) was added dropwise to above solution. After stirring at this temperature for 1 h, the mixture was cooled to 0° C. for 3 h. The solids were collected by filtration and dried under vacuum to afford the title compound (36.0 g, 84% yield). MS (ESI) calc'd for (C₈H₈BrNO₂) [M+H]⁺, 230.0; found, 230.1.

Step 2: Synthesis of N-(5-bromo-2-hydroxy-3-nitrophenyl)acetamide. To a mixture of N-(5-bromo-2-hydroxyphenyl)acetamide (20.0 g, 86.93 mmol) in dichloromethane (300 mL) was added acetic anhydride (17.7 g, 173.87 mmol). Then nitric acid (9.0 g, 95.63 mmol, 67%) was added slowly to above solution at 0° C. The mixture was stirred at 0-5° C. for 2 h. The mixture was poured into cold petroleum ether (500 mL). The solids were collected by filtration. The solids (combined with another 10 g crude solid) were crystallized with ethanol to afford the title compound (27.0 g, 60% yield). MS (ESI) calc'd for (C₈H₇BrN₂O₄) [M+H]⁺, 274.9; found, 275.1.

Step 3: Synthesis of 5-bromo-2-methyl-7-nitro-1,3-benzoxazole. To a solution of N-(5-bromo-2-hydroxy-3-nitrophenyl)acetamide (27.0 g, 98.16 mmol) in dichloroethane (500 mL) was added POCl₃ (30.1 g, 196.32 mmol) at rt. Then the mixture was heated at reflux for 16 h. The mixture was poured into ice water and basified with saturated sodium bicarbonate aqueous solution to pH 8. The organic layer was removed under reduced pressure. The solids were collected by filtration, and dried under vacuum. The solids were purified by Chromatography A twice to afford the title compound (16.0 g, 63% yield). MS (ESI) calc'd for (C₈H₅BrN₂O₃) [M+H]⁺, 256.9; found, 257.1.

Step 4: Synthesis of 1-(2-methyl-7-nitro-1,3-benzoxazol-5-yl)ethan-1-one. To a degassed solution of 5-bromo-2-methyl-7-nitro-1,3-benzoxazole (10.0 g, 38.90 mmol) and tributyl(1-ethoxyethenyl)stannane (16.9 g, 46.69 mmol) in dry dioxane (200 mL) was added Pd(PPh₃)₂Cl₂ (2.7 g, 3.89 mmol). Then the mixture was heated at 100° C. for 16 h. The mixture was cooled to rt, then added to acetic acid (30% aqueous, 300 mL) at 0° C. The mixture was then warmed to rt for 6 h. following General Work-up Procedure 1, the resulting residue was purified by Chromatography A to afford the title compound (5.0 g, 58% yield). MS (ESI) calc'd for (C₁₀H₈N₂O₄) [M+H]⁺, 221.0; found, 221.1.

Step 6: Synthesis of l-(2-methyl-7-nitro-1,3-benzoxazol-5-yl)ethan-1-ol. To a suspension of LiAlH₄ (1.7 g, 45.42 mmol) in anhydrous THE (100 mL) was added a solution of 1-(2-methyl-7-nitro-1,3-benzoxazol-5-yl)ethan-1-one (5.0 g, 22.71 mmol) in THF (100 mL) at −78° C. The mixture was stirred at −78° C. for 1 h. The reaction was quenched with water, followed by General Work-up Procedure 1 and the resulting residue was purified by Chromatography A to afford the title compound (1.7 g, 33% yield).

Step 7: Synthesis of 1-(2-methyl-7-nitro-1,3-benzoxazol-5-yl)ethyl methanesulfonate. To a solution of l-(2-methyl-7-nitro-1,3-benzoxazol-5-yl)ethan-1-ol (1.7 g, 7.65 mmol) and N,N-diisopropylethylamine (5.9 g, 45.91 mmol) in dichloromethane (100 mL) was added MsCl (2.6 g, 22.95 mmol) slowly at rt. The mixture was stirred at rt for 16 h. The mixture was concentrated to afford the title compound (2.5 g). MS (ESI) calc'd for (C₁₁H₁₂N₂O₆S) [M+H]⁺, 301.0; found, 301.2.

Step 8: Synthesis of 2-methyl-5-[1-[(4-methyl-4H-1,2,4-triazol-3-yl)sulfanyl]ethyl]-7-nitro-1,3-benzoxazole. A mixture of l-(2-methyl-7-nitro-1,3-benzoxazol-5-yl)ethyl methanesulfonate (2.5 g, crude), 4-methyl-4H-1,2,4-triazole-3-thiol (1.9 g, 16.65 mmol) and potassium carbonate (3.5 g, 24.98 mmol) in acetonitrile (100 mL) was heated at 80° C. for 3 h. Following General Work-up Procedure 1, the resulting residue was purified by Chromatography B to afford the title compound (1.2 g, 45% yield over two steps). MS (ESI) calc'd for (C₁₃H₁₃N₅O₃S) [M+H]⁺, 320.1; found, 320.2.

Step 9: Synthesis of 2-methyl-5-[1-[(4-methyl-4H-1,2,4-triazol-3-yl)sulfanyl]ethyl]-1,3-benzoxazol-7-amine (B-1). To a solution of 2-methyl-5-[1-[(4-methyl-4H-1,2,4-triazol-3-yl)sulfanyl]ethyl]-7-nitro-1,3-benzoxazole (1.1 g, 3.44 mmol) in methanol (20 mL) was added palladium on carbon (1.0 g) at rt. Then the mixture was stirred at rt for 20 h under hydrogen. The solids were filtered off. The filtrate was concentrated to afford B-1 (620 mg, 70% purity). MS (ESI) calc'd for (C₁₃H₁₅N₅OS) [M+H]⁺, 290.1; found, 290.2.

### Example C

### (S)-4-(1-((4-methyl-4H-1,2,4-triazol-3-yl)thio)ethyl)pyridin-2-amine (C-1)

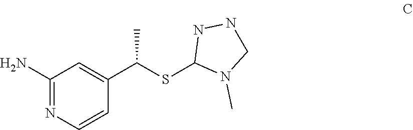 [SMILES: C[C@H](SC1NNCN1C)c1ccnc(N)c1]

Step 1: Synthesis of methyl 2-((tert-butoxycarbonyl)amino)isonicotinate (C-2). To a mixture of methyl 2-aminoisonicotinate (200 g, 1.32 mol) and di-tert-butyl dicarbonate (430.0 g, 1.97 mol) in t-BuOH (800 mL) and acetone (2400 mL) was added N,N-dimethylpyridin-4-amine (9.6 g, 78.6 mmol) in portions. The mixture was stirred at rt for 16 h and diluted with hexane (600 mL). The mixture was cooled to 0° C., the precipitated product was collected and dried to give C-2 (239.0 g, 72%). MS (ESI) calculated for (C₁₂H₁₆N₂O₄) [M+H]⁺, 253.1; found, 253.1.

Step 2: Synthesis of 2-((tert-butoxycarbonyl)amino)isonicotinic acid (C-3). To a solution of C-2 (239.0 g, 0.95 mol) in THF (2400 mL) was added a solution of lithium hydroxide (45.6 g, 1.9 mol) in water (600 mL). The mixture was stirred at rt overnight, and then diluted with water (1500 mL). Most of the THF was removed under reduced pressure. The pH of the mixture was adjusted to 3 with citric acid (saturated). The precipitated product was collected and dried to give C-3 (253.0 g, crude). MS (ESI) calculated for (C₁₁H₁₄N₂O₄) [M−H]⁻, 237.1; found, 237.0.

Step 3: Synthesis of tert-butyl (4-(methoxy(methyl)carbamoyl)pyridin-2-yl)carbamate (C-4). To a mixture of C-3 (253.0 g, 1.06 mol), N,O-dimethylhydroxylamine hydrochloride (103.1 g, 1.06 mol) and N,N-diisopropylethylamine (548.9 g, 4.25 mol) in dry DMF (3 L) was added HATU (484.8 g, 1.28 mol) at 0° C. The mixture was stirred at rt for 1 h, and diluted with water. Following General Work-up Procedure 1, the residue was triturated with EtOAc/petroleum ether (1:9) to afford C-4 (236.0 g, 88% over two steps). MS (ESI) calculated for (C₁₃H₁₉N₃O₄) [M+H]⁺, 282.1; found, 282.1.

Step 4: Synthesis of tert-butyl (4-acetylpyridin-2-yl)carbamate (C-5). To a stirred solution of C-4 (236.0 g, 0.84 mol) in anhydrous THF (3 L) was added MeMgBr (840 mL, 2.52 mol, 3 M in THF) dropwise at 0° C. under nitrogen. The mixture was stirred at 0° C. for 1 h, and then quenched with aqueous ammonium chloride (saturated) carefully. Following General Work-up Procedure 1, the resulting residue was triturated with EtOAc:petroleum ether (1:8) to afford C-5 (160.0 g, 80%). MS (ESI) calculated for (C₁₋₂H₁₆N₂O₃) [M+H]⁺, 237.1; found, 237.1.

Step 5: Synthesis of tert-butyl (4-(l-hydroxyethyl)pyridin-2-yl)carbamate (C-6). To a solution of C-5 (140.0 g, 0.59 mol) in methanol (1400 mL) was added sodium borohydride (27.1 g, 0.71 mol) in portions at 0° C. The mixture was stirred at 0° C. for 1.5 h and quenched with water. Most of the methanol was removed under reduced pressure. Following General Work-up Procedure 1, the resulting residue was purified by Chromatography A to afford C-6 (139.0 g, 98%) as a colorless solid.

Step 6: Synthesis of (R)-1-(2-((tert-butoxycarbonyl)amino)pyridin-4-yl)ethyl acetate (C-7) and tert-butyl (S)-(4-(1-hydroxyethyl)pyridin-2-yl)carbamate (C-8a). To a mixture of C-6 (40.0 g, 0.17 mol) and vinyl acetate (144.6 g, 1.68 mol) in diisopropyl ether (2 L) was added Novozym 435 (4.0 g, 10% w/w). The mixture was stirred at 35° C. for 16 h. The reaction was monitored by LCMS, when the ratio of (C-7):(C-8a) was ˜1:1, the mixture was filtered. The filtrate was evaporated in vacuo. The residue was purified by Chromatography A to afford C-7 (23.0 g) and (C-8a) (19.0 g, e.e=98.8%, determined using ChiralPak AD column).

Step 7: Synthesis of tert-butyl (R)-(4-(l-hydroxyethyl)pyridin-2-yl)carbamate (C-8b). To a solution of C-7 (23 g, 82.1 mmol) in methanol (250 mL) was added potassium carbonate (22.6 g, 164 mmol) at 20° C. The mixture was stirred at rt for 1.5 h. The solid was filtered off, and the filtrate was evaporated in vacuo. The residue was purified by Chromatography A to afford C-8b (17.8 g, 91%). MS (ESI) calculated for (C₁₂H₁₈N₂O₃) [M+H]⁺, 239.1; found, 239.1. ¹H NMR (300 MHz, DMSO-d₆) δ 9.66 (s, 1H), 8.14 (dd, J₁=5.1 Hz, J₂=0.9 Hz, 1H), 7.83 (dt, J₁=1.5 Hz, J₂=0.9 Hz, 1H), 6.97 (ddd, J₁=5.1 Hz, J₂=1.5 Hz, J₃=0.6 Hz, 1H), 5.36 (d, J=4.5 Hz, 1H), 4.76-4.61 (m, 1H), 1.48 (s, 9H), 1.31 (d, J=6.6 Hz, 3H).

Step 8: Synthesis of tert-butyl (S)-(4-(1-((4-methyl-4H-1,2,4-triazol-3-yl)thio)ethyl)pyridin-2-yl)carbamate (C-9). To a mixture of C-8b (7.8 g, 32.78 mmol), 4-methyl-4H-1,2,4-triazole-3-thiol (4.52 g, 39.33 mmol) and triphenylphosphine (12.9 g, 49.16 mmol) in dry THF (200 mL) was added diisopropyl azodicarboxylate (9.9 g, 49.16 mmol) at 0° C. under nitrogen. The mixture was stirred at rt for 16 h, quenched with water. Following General Work-up Procedure 1, the resulting residue was purified by Chromatography B to afford C-9 (9.5 g, 86%) as a yellow oil. MS (ESI) calculated for (C₁₅H₂₁N₅C₂S) [M+H]⁺, 336.1; found, 336.0.

Step 9: Synthesis of (S)-4-(1-((4-methyl-4H-1,2,4-triazol-3-yl)thio)ethyl)pyridin-2-amine (C-1) HCl salt. A mixture of C-9 (9.5 g, 28.35 mol) in hydrochloric acid/1,4-dioxane (4 M, 40 mL) was stirred at rt for 2-6 h and evaporated in vacuo to afford C-1·HCl (5.3 g, crude) as a yellow semi-solid, which was used without purification. MS (ESI) calculated for (C₁₀H₁₃N₅S) [M+H]⁺, 236.1; found, 236.1. ¹H NMR (300 MHz, Methanol-d₄) δ 9.78 (s, 1H), 7.87 (dd, J₁=6.9 Hz, J₂=0.6 Hz, 1H), 7.18-7.04 (m, 2H), 5.02-4.92 (m, 1H), 3.87 (s, 3H), 1.82 (d, J=7.2 Hz, 3H).

### Example D

### 3-(1-(4-methyl-4H-1,2,4-triazol-3-yl)propan-2-yl)aniline (D-1)

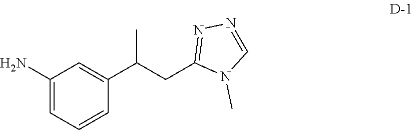 [SMILES: CC(Cc1nncn1C)c1cccc(N)c1]

Step 1: Synthesis of 3-(3-nitrophenyl)butanehydrazide. A mixture of ethyl 3-(3-nitrophenyl)but-2-enoate (WO2010047372) (50.0 g, 191 mmol) and hydrazine hydrate (100.0 g, 2.00 mol) in ethanol (1000 mL) was heated for 72 h at 80° C. under O₂. The mixture was evaporated in vacuo. The residue was dissolved with water, followed by General Work-up Procedure 1 to afford title compound (25.0 g, crude) as a light yellow oil, which was used without purification. MS (ESI) calculated for (C₁₀H₁₃N₃O₃) [M+H]⁺, 224.1; found, 224.0.

Step 2: Synthesis of N,N-dimethyl-N′-(3-(3-nitrophenyl)butanoyl)formohydrazon-amide. To a 3-(3-nitrophenyl)butanehydrazide (30.0 g, crude) in dichloromethane (350 mL) was added N, A-dimethylformamide dimethyl acetal (60.0 g, 504.2 mmol) at rt. The mixture was refluxed for 3 h before concentration. The residue was purified by Chromatography B to afford the title compound (14.0 g, 88% purity on LCMS), which was used without purification. MS (ESI) calculated for (C₁₃H₁₈N₄O₃) [M+H]⁺, 279.1; found, 279.0.

Step 3: Synthesis of 4-methyl-3-(2-(3-nitrophenyl)propyl)-4H-1,2,4-triazole. To a solution of N,N-dimethyl-N′-(3-(3-nitrophenyl)butanoyl)formohydrazonamide (50.0 g, crude) in acetic acid (150 mL) was added methylamine (150 mL, 2 M in THF). The mixture was stirred at 90° C. for 3 h and then concentrated. The residue was diluted with water. The pH value of the mixture was adjusted to 8 with saturated sodium bicarbonate solution. The mixture was extracted with dichloromethane, washed with brine, dried, and filtered. The filtrate was evaporated in vacuo. The residue was purified by Chromatography to give the title compound (12.0 g, 6.4% over 3 steps) as a yellow oil. MS (ESI) calculated for (C₁₂H₁₄N₄O₂) [M+H]⁺, 247.1; found, 247.1.

Step 4: Synthesis of 3-(1-(4-methyl-4H-1,2,4-triazol-3-yl)propan-2-yl)aniline (D-1). To a solution of 4-methyl-3-(2-(3-nitrophenyl)propyl)-4H-1,2,4-triazole (12.0 g, 48.80 mmol) in ethanol (10 mL) was added palladium on carbon (2.4 g, wet, 10%). The mixture was stirred at rt for 16 h under hydrogen. The mixture was filtered. The filtrate was evaporated in vacuo. The residue was purified by Chromatography B to afford the product. (7.6 g, 72%) as a yellow oil.

### (R)-3-(1-(4-methyl-4H-1,2,4-triazol-3-yl)propan-2-yl)aniline (D-a)

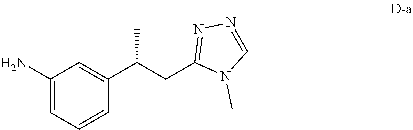 [SMILES: C[C@H](Cc1nncn1C)c1cccc(N)c1]

Step 1: Synthesis of (R,E)-3-(3-(3-nitrophenyl)acryloyl)-4-phenyloxazolidin-2-one. A mixture of (E)-3-(3-nitrophenyl)acrylic acid (500.0 g, 2.59 mol) and SOCl₂ (2.5 L) was heated at 80° C. for 2 h. Then the mixture was concentrated to afford (E)-3-(3-nitrophenyl)acryloyl chloride as a light yellow solid. In another three-necked flask was placed a solution of (R)-4-phenyloxazolidin-2-one (422.4 g, 2.59 mol) in anhydrous THF (1.0 L). Then LiHMDS (3.1 L, 3.10 mol, 1M in THF) was added dropwise to above solution at −70° C. under nitrogen atmosphere. After stirring at −70° C. for 30 min, a solution of (E)-3-(3-nitrophenyl)acryloyl chloride in anhydrous THF (1 L) was added dropwise to above mixture at −70° C. The mixture was warmed to 0° C. in 1 h. The reaction was quenched with saturated ammonium chloride aqueous solution at 0° C. Following General Work-up Procedure 1, the resulting residue was purified by trituration with petroleum ether/ethyl acetate (3/2) to afford the title compound (480.0 g, 55%) as a light yellow solid. MS (ESI) calc'd for (C₁₈H₁₄N₂O₅) [M+H]⁺, 339.1; found, 339.1.

Step 2: Synthesis of (R)-3-((R)-3-(3-nitrophenyl)butanoyl)-4-phenyloxazolidin-2-one. To a suspension of CuBrMe₂S (314.9 g, 1.54 mol) in anhydrous THF (1.0 L) was added MeMgBr (1.0 L, 3.00 mol, 3 M in 2-methylTHF) dropwise with stirring at −40° C. under nitrogen atmosphere. The mixture was allowed to warm to −30˜−20° C. for 40 min. Then The mixture was cooled to −40° C., and to this was added BF₃Et₂O (200.3 g, 1.54 mol) dropwise with stirring at −40° C. Then the mixture was warmed to −30˜−20° C. over 40 min. The mixture was cooled to −40° C. again, to this was added a suspension of (R,E)-3-(3-(3-nitrophenyl)acryloyl)-4-phenyloxazolidin-2-one (350.0 g, 1.03 mol) in anhydrous THF (1.0 L) slowly with stirring at −40˜−30° C. The mixture was allowed to warm to −20° C. for 2 h. The reaction was then quenched by saturated ammonium chloride aqueous solution. Following General Work-up Procedure 1, the resulting crude product was precipitated by the addition of petroleum ether. The solids were collected by filtration, then triturated with methanol to afford the title compound (210.0 g, 57%) as a yellow solid. MS (ESI) calc'd for (C₁₉H₁₈N₂O₅) [M+H]⁺, 355.1; found, 355.1.

Step 3: Synthesis of (R)-3-(3-nitrophenyl)butanehydrazide. To a solution of (R)-3-((R)-3-(3-nitrophenyl)butanoyl)-4-phenyloxazolidin-2-one (160.0 g, 451.52 mmol) in THF (1.5 L) was added hydrazine hydrate (56.5 g, 903.05 mmol, 80%) dropwise at 0° C. The mixture was stirred at rt for 16 h. The mixture was concentrated. The residue was diluted with water, followed by General Work-up Procedure 1 and the resulting to afford the title compound (160.0 g, crude) as a brown semi-solid. MS (ESI) calc'd for (C₁₀H₁₃N₃O₃) [M+H]⁺, 224.1; found, 224.1.

Step 4: Synthesis of (R,E)-N,N-dimethyl-N′-(3-(3-nitrophenyl)butanoyl)formohydrazonamide. To a stirred solution of (R)-3-(3-nitrophenyl)butanehydrazide (160.0 g, 716.75 mmol, crude) in DCM (1.5 L) was added dimethylformamide dimethyl acetal (170.8 g, 1.43 mol) at rt. The mixture was heated at 50° C. for 2 h. The mixture The mixture was concentrated to afford the title compound (160.0 g, crude) as a brown syrup. MS (ESI) calc'd for (C₁₃H₁₈N₄O₃) [M+H]⁺, 279.1; found, 279.2.

Step 5: Synthesis of (R)-4-methyl-3-(2-(3-nitrophenyl)propyl)-4H-1,2,4-triazole (D-6a): To a stirred solution of (R,E)-N,N-dimethyl-N′-(3-(3-nitrophenyl)butanoyl)formohydrazonamide (160.0 g, 0.40 mol, crude) in acetic acid (2.0 L) was added methylamine (2.0 L, 4.00 mol, 2 M in THF) at 0˜10° C. The mixture heated at 90° C. for 3 h. The mixture was concentrated. The residue was diluted with water and basified with Na₂CO₃ (aqueous) to pH 7-8. Following General Work-up Procedure 1, the resulting residue was purified by Chromatography D with methanol in EtOAc to afford the title compound (58.0 g, 41% over three steps) as a brown syrup. MS (ESI) calc'd for (C₁₂H₁₄N₄O₂) [M+H]⁺, 247.1; found, 247.2.

Step 6: Synthesis of (R)-3-(1-(4-methyl-4H-1,2,4-triazol-3-yl)propan-2-yl)aniline (D-a): To a solution of (R)-4-methyl-3-(2-(3-nitrophenyl)propyl)-4H-1,2,4-triazole (58.0 g, 235.52 mmol) in ethanol (600.0 mL) was added palladium on carbon (6.0 g) at rt under nitrogen atmosphere. Then the mixture was stirred at rt for 16 h under a hydrogen atmosphere. The mixture was filtered. The filtrate was concentrated. The desired D-a (43.0 g, 84%) was obtained as a yellow solid using Chromatography Chromatography A purification methods. MS (ESI) calc'd for (C₁₂H₁₆N₄) [M+H]⁺, 217.1; found, 217.0.

### (S)-3-(1-(4-methyl-4H-1,2,4-triazol-3-yl)propan-2-yl)aniline (D-b)

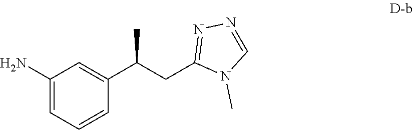 [SMILES: C[C@@H](Cc1nncn1C)c1cccc(N)c1]

Racemic D-1 (7.3 g) was separated by prep-chiral SFC to afford D-b (2.8 g) and D-a (2.8 g) over a CHIRALPAK-AS column eluting with MeOH in CO₂.

### Example E

### (R)-3-(1-(4H-1,2,4-triazol-3-yl)propan-2-yl)aniline (E)

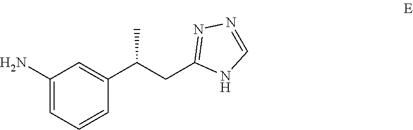

Step 1: Synthesis of (R)-3-(3-nitrophenyl)butanamide. To a solution of (R)-3-((R)-3-(3-nitrophenyl)butanoyl)-4-phenyloxazolidin-2-one (step 2, synthesis D-a) (50.0 g, 0.14 mol) in THF (500 mF) was added ammonia in water (200 mF, aqueous, 30%). The mixture was stirred at rt for 20 h. The mixture was concentrated. The residue was purified by Chromatography A to afford the title compound (22.0 g, 70%) as a brown solid. MS (ESI) calc'd for (C₁₀H₁₂N₂O₃) [M+H]⁺, 209.1; found, 209.1.

Step 2: Synthesis of (R)-N-((dimethylamino)methylene)-3-(3-nitrophenyl)butanamide. A mixture of f (R)-3-(3-nitrophenyl)butanamide (15.0 g, 72.11 mmol) and N, N-dimethylformamide dimethyl acetal (86.0 g, 72.12 mmol) in anhydrous DMF was stirred at 100° C. for 3 h, and then concentrated to afford the title compound (20.0 g, crude) as a brown oil. MS (ESI) calc'd for (C₁₃H₁₇N₃O₃) [M+H]⁺, 264.1; found, 264.1.

Step 3: Synthesis of (R)-3-(2-(3-nitrophenyl)propyl)-4H-1,2,4-triazole. To a solution of (R)-N-((dimethylamino)methylene)-3-(3-nitrophenyl)butanamide (20.0 g, crude) in glacial acetic acid (240 mF) was added hydrazine hydrate (24.0 g, 0.38 mol). The mixture was heated at 90° C. for 2 h. The mixture was concentrated under reduced pressure. The residue was diluted with water and basified by saturated potassium carbonate aqueous solution to pH 8. The mixture was extracted with DCM (3×). The organic layers were dried, filtered, and concentrated. The residue was purified by Chromatography A to afford the title compound (10.0 g, 32%) as a yellow solid. MS (ESI) calc'd for (C₁₁H₁₂N₄O₂) [M+H]⁺, 233.1; found, 233.1.

Step 4: Synthesis of (R)-3-(1-(4H-1,2,4-triazol-3-yl)propan-2-yl)aniline (E). To a solution of (R)-3-(2-(3-nitrophenyl)propyl)-4H-1,2,4-triazole (4.6 g, 4.74 mmol) in methanol (50 mL) was added palladium (1.0 g, wet, 5% on carbon). The flask was evacuated and backfilled with hydrogen. The mixture was stirred at rt for 16 h under a hydrogen atmosphere. The solids were filtered off. The filtrate was concentrated to afford the title compound E (3.0 g, crude) as a yellow solid. MS (ESI) calc'd for (C₁₁H₁₄N₄) [M+H]⁺, 203.1; found, 203.1.

### Example F

### Synthesis of 3-(1-(4-methyl-1H-pyrazol-3-yl)propan-2-yl)aniline (F)

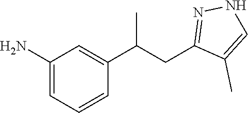 [SMILES: Cc1c[nH]nc1CC(C)c1cccc(N)c1]

Step 1: Synthesis of 3-(3-(tert-butoxycarbonylamino)phenyl)butanoic acid. A mixture of ethyl 3-(3-[[(tert-butoxy)carbonyl]amino]phenyl)butanoate (66.0 g, 214.71 mmol) and LiOH (31.0 g, 1.29 mol) in THF (900 mL) and water (300 mL) was stirred at rt for 24 h. The organic solvent was removed under vacuum. The mixture was acidified by hydrochloric acid (aq., 2M) to pH 4 followed by General Work-up Procedure 1 to afford the title compound (63.0 g, crude) as a light yellow oil. MS (ESI) calc'd for (C₁₅H₂₁NO₄) [M+H]⁺, 280.1; found, 280.2].

Step 2: Synthesis of methyl 5-(3-(tert-butoxycarbonylamino)phenyl)-3-oxohexanoate. A mixture of 3-(3-[[(tert-butoxy)carbonyl]amino]phenyl)butanoic acid (55.0 g, 196.90 mmol) and N-N′-carbonyl diimidazole (35.1 g, 216.47 mmol) in acetonitrile (500 mL) was stirred at 50° C. for 1 h. To a mixture of MgCl₂ (46.8 g, 492.25 mmol), potassium 3-methoxy-3-oxopropanoate (61.5 g, 393.78 mmol) and TEA (59.7 g, 590.70 mmol) in acetonitrile (1500 mL) was added to the above mixture slowly at rt. The mixture was stirred at 50° C. for 16 h. The solids were filtered off. The filtrate was concentrated. The residue was purified by Chromatography A to afford the title compound (36.5 g, 55%) as a colorless solid. MS (ESI) calc'd for (C₁₈H₂₅NO₅) [M+H]⁺, 336.2; found, 336.3.

Step 3: Synthesis of methyl 5-(3-(tert-butoxycarbonylamino)phenyl)-2-((dimethylamino)methylene)-3-oxohexanoate. A mixture of methyl 5-(3-[[(tert-butoxy)carbonyl]amino]phenyl)-3-oxohexanoate (31.5 g, 93.92 mmol). N,N-dimethylformamide dimethyl acetal (13.98 g, 117.40 mmol) in DMF (320 mL) was stirred at 40° C. for 3 h. General Work-up Procedure 1 was followed to afford the title compound (44.0 g, crude) as a light yellow oil, used without purification. MS (ESI) calc'd for (C₂₁H₃₀N₂O₅) [M+H]⁺, 391.2; found, 391.3.

Step 4: Synthesis of methyl 3-[2-(3-[[(tert-butoxy)carbonyl]amino]phenyl)propyl]-1H-pyrazole-4-carboxylate. A mixture of methyl 5-(3-[[(tert-butoxy)carbonyl]amino]phenyl)-2-[(dimethylamino)methylidene]-3-oxohexanoate (44 g, 112.68 mmol) and hydrazine hydrate (28.2 g, 80%) in ethanol (500 mL) was stirred at 85° C. for 3 h. The mixture was concentrated. The residue was purified by Chromatography A to afford the title compound (34 g, 84%) as a light yellow oil. MS (ESI) calc'd for (C₁₉H₂₅N₃O₄) [M+H]⁺, 360.2; found, 360.2.

Step 5: Synthesis of tert-butyl N-(3-[1-[4-(hydroxymethyl)-1H-pyrazol-3-yl]propan-2-yl]phenyl)carbamate. To a mixture of methyl 3-[2-(3-[[(tert-butoxy)carbonyl]amino]phenyl)propyl]-1H-pyrazole-4-carboxylate (34.0 g, 94.6 mmol) in anhydrous THF (500 mL) was added DIBAL-H (566 mL, 1 M in THF) dropwise at −60° C. The mixture was warmed at rt for 3 h. The reaction was then quenched by saturated ammonium chloride aqueous solution followed by General Work-up Procedure 1, the resulting residue was purified by Chromatography A to afford the title compound (23 g, 73%) as a colorless solid. MS (ESI) calc'd for (C₁₈H₂₅N₃O₃) [M+H]⁺, 332.2; found, 332.0.

Step 6: Synthesis of 3-[1-(4-methyl-1H-pyrazol-3-yl)propan-2-yl]aniline (F). To a mixture of tert-butyl N-(3-[1-[4-(hydroxymethyl)-1H-pyrazol-3-yl]propan-2-yl]phenyl)carbamate (18.0 g, 54.3 mmol) and TEA (16.4 g, 162.3 mmol) in dichloromethane (200 mL) was added MsCl (14 g, 123.8 mmol) dropwise at 0° C. The mixture was stirred at rt for 2 h. The mixture was washed with brine. The organic layer was dried, filtered and concentrated. The residue was dissolved in DMSO (200 mL) and sodium borohydride (6.1 g, 162.3 mmol) was added to above mixture. The mixture was stirred at 80° C. for 16 h and potassium hydroxide (14.3 g, 246.5 mmol) was added to above mixture at rt and stirred at 80° C. for another 16 h. Following General Work-up Procedure 1, the desired compound F (4 g, 40%) was obtained as a colorless oil using Chromatography B. MS (ESI) calc'd for (C₁₃H₁₇N₃) [M+H]⁺, 216.1; found, 216.1.

### Example G

### Synthesis of 3-(1-(4-methylisoxazol-3-yl)propan-2-yl)aniline (G)

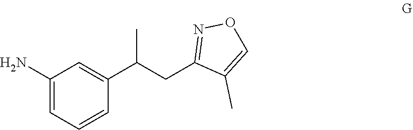 [SMILES: Cc1conc1CC(C)c1cccc(N)c1]

Step 1: Synthesis of ethyl 3-(3-aminophenyl)butanoate. To a solution of ethyl-3-(3-nitrophenyl)but-2-enoate (step 1 of intermediate D-1) (33.0 g, 0.14 mol) in ethanol (500 mL) was added palladium (5.0 g, wet, 5% on carbon) under nitrogen atmosphere. The flask was evacuated and backfilled with hydrogen. The mixture was stirred at rt for 16 h under hydrogen atmosphere. The reaction was repeated three batches, and then combined. The combined reaction mixture was filtered and concentrated to afford the title compound (71.0 g, crude) as a yellow oil. MS (ESI) calc'd for (C₁₋₂H₁₇NO₂) [M+H]⁺, 208.1; found, 208.1.

Step 2: Synthesis of ethyl 3-(3-(tert-butoxycarbonylamino)phenyl)butanoate. A mixture of ethyl 3-(3-aminophenyl)butanoate (71.0 g, 0.34 mol) and di-tert-butyl dicarbonate (73.1 g, 0.34 mol) in anhydrous THF (1 L) was heated at reflux for 3 h. Then the mixture was concentrated. The residue was purified by Chromatography A to afford the title compound (113.0 g, 87% over two steps) as a yellow oil. MS (ESI) calc'd for (C₁₇H₂₅NO₄) [M+H]⁺, 308.2; found, 308.1.

Step 3: Synthesis of tert-butyl 3-(4-hydroxybutan-2-yl)phenylcarbamate. To a suspension of LiAlH₄ (21.0 g, 0.55 mol) in anhydrous THF (500 mL) was added a solution of ethyl 3-(3-(tert-butoxycarbonylamino)phenyl)butanoate (113.0 g, 0.37 mol) in anhydrous THF (300 mL) slowly at −30° C. Then the mixture was stirred at −30° C. for 3 h. The reaction was quenched with water (20 mL) at −30 to 0° C. The solids were filtered off. Following General Work-up Procedure 1 afforded the title compound (84.0 g, 86%) as a yellow oil. MS (ESI) calc'd for (C₁₅H₂₃NO₃) [M+H]⁺, 266.2; found, 266.2.

Step 4: Synthesis of tert-butyl 3-(4-oxobutan-2-yl)phenylcarbamate. To a solution of tert-butyl 3-(4-hydroxybutan-2-yl)phenylcarbamate (64.0 g, 0.24 mol) in anhydrous THF (1.0 L) was added Dess-Martin periodinane (152.6 g, 0.36 mol) in portions at rt. After stirring for 2 h, the reaction was quenched by sodium bicarbonate (saturated)/sodium thiosulfate (saturated). The solids were filtered off. Following General Work-up Procedure 1, the residue was purified by Chromatography A to afford the title compound (36.5 g, 57%) as a yellow oil. MS (ESI) calc'd for (C₁₅H₂₁NO₃) [M+H]⁺, 264.1; found, 264.2.

Step 5: Synthesis of tert-butyl 3-(4-(hydroxyimino)butan-2-yl)phenylcarbamate. To a solution of tert-butyl 3-(4-oxobutan-2-yl)phenylcarbamate (32.0 g, 121.5 mmol) in methanol/water (400 mL/80 mL) were added hydroxylamine hydrochloride (16.8 g, 237 mmol) and sodium carbonate (25.2 g, 237 mmol). The mixture was stirred at rt for 3 h. The mixture was concentrated. The residue was diluted with water followed by General Work-up Procedure 1. The crude product was purified by Chromatography A to afford the title compound (28.0 g, 83%) as a yellow oil. MS (ESI) calc'd for (C₁₅H₂₂N₂O₃) [M+H]⁺, 279.2; found, 279.2.

Step 6: Synthesis of tert-butyl 3-(4-chloro-4-(hydroxyimino)butan-2-yl)phenylcarbamate. To a solution of tert-butyl 3-(4-(hydroxyimino)butan-2-yl)phenylcarbamate (50.0 g, 179.08 mmol) in DMF (600 mL) was added N-chlorosuccinimide (23.9 g, 179.02 mmol) in portions at 0° C. The mixture was stirred at rt for 2 h. The mixture was diluted with water followed by General Work-up Procedure 1 to afford the title compound (65.0 g, crude) as a yellow oil.

Step 7: Synthesis of ethyl 3-(2-(3-(tert-butoxycarbonylamino)phenyl)propyl)isoxazole-4-carboxylate. To a solution of tert-butyl 3-(4-chloro-4-(hydroxyimino)butan-2-yl)phenylcarbamate (33.0 g, 52.75 mmol, crude product from preceding step) and TEA (3.2 g, 31.52 mmol) in ethanol (300 mL) was added (E)-ethyl 3-(dimethylamino)acrylate (7.5 g, 52.66 mmol). After stirring at rt for 2 h, The mixture was concentrated. The crude product was purified by Chromatography A to afford the title compound (15.0 g, 76%) as a yellow oil. MS (ESI) calc'd for (C₂₀H₂₆N₂O₅) [M+H]⁺, 375.2; found, 375.1.

Step 8: Synthesis of tert-butyl (3-(1-(4-(hydroxymethyl)isoxazol-3-yl)propan-2-yl)phenyl)carbamate. To a solution of ethyl 3-(2-(3-(tert-butoxycarbonylamino)phenyl)propyl)-isoxazole-4-carboxylate (5.0 g, 13.35 mmol) in anhydrous THF (50 mL) was added DIBAL-H (80 mL, 80.00 mmol, 1 M in THF) dropwise at −70° C. The mixture warmed to 0° C. for 2 h. and was quenched by the addition of saturated aqueous ammonium chloride solution. Following General Work-up Procedure 1, the resulting crude product was purified by Chromatography A to afford the title compound (4.0 g, 90%) as a colorless oil.

Step 9: Synthesis of tert-butyl 3-(1-(4-methylisoxazol-3-yl)propan-2-yl)phenylcarbamate (G-10). To a solution of tert-butyl (3-(1-(4-(hydroxymethyl)isoxazol-3-yl)propan-2-yl)phenyl)carbamate (3.0 g, 9.03 mmol) and TEA (1.8 g, 18.08 mmol) in dichloromethane (30 mL) was added methanesulfonyl chloride 1.3 g, 10.84 mmol) dropwise at 0° C. After stirring at 0° C. for 1 h, the reaction was quenched with water, and was extracted with dichloromethane twice. The organic layers were dried, filtered and concentrated. The residue was dissolved in DMSO (30 mL), followed by the addition of sodium borohydride (0.7 g, 18.42 mmol) at rt and stirred at rt for 1 h. Following General Work-up Procedure 1, the resulting crude product was purified by Chromatography A to afford the title compound (1.8 g, 63%) as a colorless oil.

Step 10: Synthesis of 3-(1-(4-methylisoxazol-3-yl)propan-2-yl)aniline (G-1). To a solution of tert-butyl 3-(1-(4-methylisoxazol-3-yl)propan-2-yl)phenylcarbamate (0.8 g, 2.52 mmol) in dichloromethane (10 mL) was added trifluoroacetic acid (2 mL). After stirring at rt for 3 h, the mixture was concentrated. The residue was diluted with water and basified with sodium bicarbonate aqueous solution to pH 8. Following General Work-up Procedure 1 afforded the title compound (0.5 g, crude) as a yellow oil.

### Separation of (R)-3-(1-(4-methylisoxazol-3-yl)propan-2-yl)aniline (G-a) and (S)-3-(1-(4-methylisoxazol-3-yl)propan-2-yl)aniline (G-b)

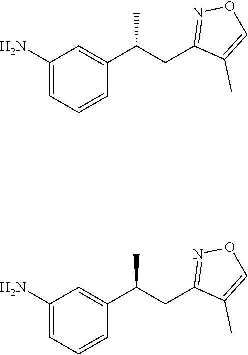 [SMILES: Cc1conc1C[C@@H](C)c1cccc(N)c1 ; Cc1conc1C[C@H](C)c1cccc(N)c1]

The racemic 3-(1-(4-methylisoxazol-3-yl)propan-2-yl)benzenamine (4 g) was separated by prep-chiral SFC over a CHIRALPAK AD-H column with MeOH and CO₂

(S)-3-(1-(4-methylisoxazol-3-yl)propan-2-yl)aniline (1.40 g): MS (ESI) calc'd for (C₁₃H₁₆N₂O) [M+1]⁺, 217.1; found, 217.0. ¹H NMR (300 MHz, DMSO-d₆) δ 8.48 (s, 1H), 6.94-6.89 (m, 1H), 6.46-6.37 (m, 3H), 4.95 (s, 2H), 2.97-2.94 (m, 1H), 2.89-2.79 (m, 2H), 1.84 (s, 3H), 1.18 (d, J=6.6 Hz, 3H).

(R)-3-(1-(4-methylisoxazol-3-yl)propan-2-yl)aniline (1.10 g): MS (ESI) calc'd for (C₁₃H₁₆N₂O) [M+1]⁺, 217.1; found, 217.0. ¹H NMR (300 MHz, DMSO-d₆) δ 8.48 (s, 1H), 6.98-6.93 (m, 1H), 6.51-6.44 (m, 3H), 5.56 (br, 2H), 2.99-2.92 (m, 1H), 2.89-2.75 (m, 2H), 1.84 (s, 3H), 1.18 (d, J=6.9 Hz, 3H).

### Example H

### Synthesis of 3-(1-(1-methyl-1H-imidazol-2-yl)propan-2-yl)aniline (H-1)

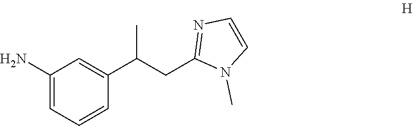 [SMILES: CC(Cc1nccn1C)c1cccc(N)c1]

Step 1: Synthesis of tert-butyl 3-(1-(1H-imidazol-2-yl)propan-2-yl)phenylcarbamate. To a solution of tert-butyl 3-(4-oxobutan-2-yl)phenylcarbamate (18.0 g, 68.4 mmol) and oxalaldehyde (19.8 g, 136.47 mmol, 40% aqueous) in methanol (200 mL) was added ammonia (95.4 g, 25% aqueous) dropwise at 0° C. The mixture was stirred at rt for 3 h. before it was concentrated. The crude product was purified by Chromatography A to afford the title compound (13.0 g, 63%) as an off-white solid.

Step 2: Synthesis of tert-butyl 3-(1-(1-methyl-1H-imidazol-2-yl)propan-2-yl)phenylcarbamate. To a solution of tert-butyl 3-(1-(1H-imidazol-2-yl)propan-2-yl)phenylcarbamate (2.0 g, 6.64 mmol) and methyl 4-methylbenzenesulfonate (1.5 g, 7.95 mmol) in anhydrous THF (20 mL) was added t-BuONa (780 mg, 8.12 mmol) in portions at 0° C. The mixture was stirred at 20° C. for 1 h. General Work-up Procedure 1 was followed. The crude product was purified by Chromatography C Chromatography to afford the title compound (1.5 g, 72%) as a yellow oil.

Step 3: Synthesis of 3-(1-(1-methyl-1H-imidazol-2-yl)propan-2-yl)aniline (H-1). A mixture of tert-butyl 3-(1-(1-methyl-1H-imidazol-2-yl)propan-2-yl)phenylcarbamate (1.0 g, 3.17 mmol) and hydrochloric acid (10 mL, 4 M in dioxane) was stirred at rt for 3 h. The mixture was concentrated to afford H-1 hydrochloride as yellow crude oil (0.9 g). MS (ESI) calc'd for (C₁₃H₁₇N₃) [M+H]⁺, 216.1; found, 216.1.

### Example J

### 2-([3-[(2R)-1-(4-methyl-4H-1,2,4-triazol-3-yl)propan-2-yl]phenyl]carbamoyl)pyridine-4-carboxylic acid (J-a) hydrogen chloride

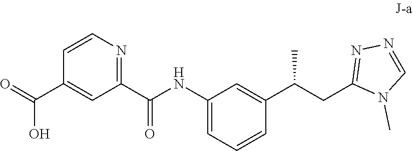 [SMILES: C[C@H](Cc1nncn1C)c1cccc(NC(=O)c2cc(C(=O)O)ccn2)c1]

Step 1: Synthesis of 4-tert-butyl 2-methyl pyridine-2,4-dicarboxylate. To a solution of 2-(methoxycarbonyl)pyridine-4-carboxylic acid (5.0 g, 27.60 mmol) and 4-dimethylaminopyridine (337 mg, 2.76 mmol) in tert-butanol (75 mL) and THF (25 mL) was added di-tert-butyl dicarbonate (12.2 g, 55.90 mmol) slowly at room temperature. The mixture was stirred at room temperature for 16 h. The mixture was concentrated under vacuum. The residue was purified by Chromatography A to afford 4-tert-butyl 2-methyl pyridine-2,4-dicarboxylate (5.6 g, 86%) as a yellow oil.

Step 2: Synthesis of 4-[(tert-butoxy)carbonyl]pyridine-2-carboxylic acid. To a solution of 4-tert-butyl 2-methyl pyridine-2,4-dicarboxylate (5.6 g, 23.60 mmol) in THF (80 mF) and water (40 mF) was added lithium hydroxide (680 mg, 28.39 mmol). The mixture was stirred at rt overnight. The mixture was acidified by hydrochloric acid (1 N) to pH 3. General Work-up Procedure 1 was followed to afford the title compound (4.7 g, 89%) as a colorless solid.

Step 3: Synthesis of tert-butyl-2-([3-[(2R)-1-(4-methyl-4H-1,2,4-triazol-3-yl)propan-2-yl]phenyl]carbamoyl)pyridine-4-carboxylate. Followed General procedure 6 with addition of reagents at 0° C. to afford tert-butyl 2-([3-[(2R)-1-(4-methyl-4H-1,2,4-triazol-3-yl)propan-2-yl]phenyl]carbamoyl)pyridine-4-carboxylate (6.0 g, 47%) as a yellow solid.

Step 4: Synthesis of 2-([3-[(2R)-1-(4-methyl-4H-1,2,4-triazol-3-yl)propan-2-yl]phenyl]carbamoyl)pyridine-4-carboxylic acid (J-a) HCl salt. A mixture of tert-butyl-2-([3-[(2R)-1-(4-methyl-4H-1,2,4-triazol-3-yl)propan-2-yl]phenyl]carbamoyl)pyridine-4-carboxylate (6.0 g, 14.23 mmol) in hydrochloric acid (4 M in dioxane, 100 mF) was stirred at rt for 16 h and the formed suspension was filtered. The filter cake was collected and dried under vacuum to afford J-a·HCl (5.5 g, crude) as a yellow solid. MS (ESI) calc'd for (C₁₉H₁₉N₅O₃) [M+H]⁺, 366.1; found, 366.0. ¹H NMR (300 MHz, DMSO-d₆) δ 10.67 (s, 1H), 9.36 (s, 1H), 8.95-8.93 (m, 1H), 8.52 (s, 1H), 8.10 (d, J=4.8, 1H), 7.81-7.79 (m, 2H), 7.33 (t, J=8.1 Hz, 1H), 7.08 (d, J=7.8 Hz, 1H), 3.70 (s, 3H), 3.38-3.27 (m, 3H), 1.36-1.31 (m, 3H).

### Example K

### 2-Chloro-4-[1-(4-methyl-4H-1,2,4-triazol-3-yl)propan-2-yl]pyridine (K)

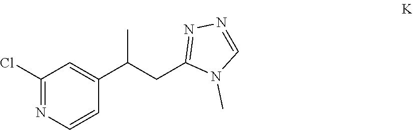 [SMILES: CC(Cc1nncn1C)c1ccnc(Cl)c1]

Step 1: Synthesis of N,2-dimethoxy-N-methylisonicotinamide. To a solution of 2-methoxyisonicotinic acid (50.0 g, 326.0 mmol) in DMF (1.0 L) was added N,O-dimethylhydroxylamine hydrochloride (63.8 g, 654.0 mmol), N,N-diisopropylethylamine (211.0 g, 1630 mmol) and HATU (248.0 g, 653.0 mmol). The resulting mixture was stirred at room temperature for 2 h. General Work-up Procedure 1 was followed. The residue was purified by Chromatography A to afford A/2-dimethoxy-A-methyl isonicotinamide (58.3 g, 91%) as a yellow oil.

Step 2: Synthesis of l-(2-methoxypyridin-4-yl)ethenone. To a solution of N,2-dimethoxy-N-methylisonicotinamide (120.0 g, 612.0 mmol) in tetrahydrofuran (2.0 L) was added methylmagnesium bromide (109 g, 918 mmol) at 0° C. under N₂. The resulting mixture was stirred at 0° C. for 3 h. Then the reaction mixture was quenched by the addition of saturated ammonium chloride aqueous solution at 0° C. and General Work-up Procedure 1 was followed. The residue was purified by chromatography A to afford the title compound (74.5 g, 81%) as a yellow oil.

Step 3: Synthesis of (E)-ethyl 3-(2-methoxypyridin-4-yl)but-2-enoate. To a solution of ethyl 2-(diethoxyphosphoryl)acetate (131.0 g, 582.0 mmol) in THF (2.0 L) was added potassioum tert-butoxide (65.2 g, 582.0 mmol) at 0° C. under N₂. The mixture was stirred at 0° C. for 30 min. Then l-(2-methoxypyridin-4-yl)ethanone (22.0 g, 145.0 mmol) was added slowly to the above mixture at 0° C. The mixture was stirred at rt for 16 h. Then the reaction was quenched by saturated ammonium chloride solution. General Work-up Procedure 1 was followed. The residue was purified by Chromatography to the title compound (39.0 g) as a yellow oil.

Step 4: Synthesis of ethyl 3-(2-methoxypyridin-4-yl)butanoate. To a solution of (E)-ethyl 3-(2-methoxypyridin-4-yl)but-2-enoate (55.0 g, 248.0 mmol) in methanol (1000 mF) was added Pd/C (5.5 g, dry) at rt under nitrogen. The mixture was stirred at rt for 12 h under H₂. The mixture was then filtered. The filtrate was evaporated in vacuo to afford the title compound (56.0 g, crude) as a yellow oil, which was used without purification.

Step 5: Synthesis of 3-(2-methoxypyridin-4-yl)butanehydrazide. To a solution of ethyl 3-(2-methoxypyridin-4-yl)butanoate (50.0 g, 224.0 mmol) in ethanol (500 mL) was added hydrazine hydrate (140 g, 80%). The mixture was stirred at 95° C. for 48 h. The solvent was evaporated in vacuo to afford the title compound (42.0 g, crude) as a yellow oil, which was used without purification.

Step 6: Synthesis of (E)-N′-(3-(2-methoxypyridin-4-yl)butanoyl)-N,N-dimethylformohydrazonamide. To a solution of 3-(2-methoxypyridin-4-yl)butanehydrazide (20.0 g, 95.50 mmol) in dichloromethane (500 mL) was added N,N-dimethylformamide dimethyl acetal (34.2 g, 287.0 mmol). The mixture was stirred at 55° C. for 2 h. The mixture was evaporated in vacuo. The residue was purified by Chromatography B to afford the title compound (19.0 g, 75%) as a yellow solid.

Step 7: Synthesis of 2-methoxy-4-(1-(4-methyl-4H-1,2,4-triazol-3-yl)propan-2-yl)pyridine. To a solution of (E)-N′-(3-(2-methoxypyridin-4-yl)butanoyl)-N,N-dimethylformohydrazonamide (7.0 g, 26.50 mmol) in acetic acid (133 mL) was added methylamine in THF (133 mL, 2 mol/L). The mixture was stirred at 90° C. for 3 h. The mixture was evaporated in vacuo. The residue was alkalized with aqueous sodium bicarbonate to pH 8-9. General Work-up Procedure 1 was followed. The residue was purified by Chromatography D with 0-5% methanol in EtOAc to afford the title compound (2.9 g, 47%) as a yellow oil.

Step 8: Synthesis of 4-(1-(4-methyl-4H-1,2,4-triazol-3-yl)propan-2-yl)pyridin-2-ol. To a solution of 2-methoxy-4-(1-(4-methyl-4H-1,2,4-triazol-3-yl)propan-2-yl)pyridine (10.0 g, 43.10 mmol) in acetic acid (170 mL) was added hydrogen bromide (170 mL, 40%) at rt. The mixture was stirred at 90° C. for 12 h. When the reaction was completed, the mixture was evaporated in vacuo. The residue was alkalized with aqueous ammonium hydroxide to pH 8-9 and then extracted with EtOAc. The aqueous phase was evaporated in vacuo to afford the title compound (15.0 g, crude) as a colorless solid, which was used without purification.

Step 9: Synthesis of 2-chloro-4-(1-(4-methyl-4H-1,2,4-triazol-3-yl)propan-2-yl)pyridine. To a solution of 4-(1-(4-methyl-4H-1,2,4-triazol-3-yl)propan-2-yl)pyridin-2-ol (30 g, crude) in acetonitrile (300 mL) was added phosphoryl trichloride (300 mL) at rt. The mixture was stirred at 90° C. for 5 h. The solvent was evaporated under reduced pressure and the residue was alkalized with aqueous sodium bicarbonate to pH 7-8, and then extracted with dichloromethane. The combined organic layers were washed with brine, dried, and filtered. The filtrate was evaporated in vacuo. The title compound (13.0 g, 40%) was obtained as a yellow oil using Chromatography B. MS (ESI) calculated for (C₁₁H₁₃ClN₄) [M+1]⁺, 237.1; found, 237.3. ¹H NMR (400 MHz, DMSO-d₆) δ 8.31-8.29 (m, 2H), 7.48 (s, 1H), 7.37-7.35 (m, 1H), 3.54 (s, 3H), 3.36-3.31 (m, 1H), 3.09-3.02 (m, 2H), 1.27 (d, J=6.8 Hz, 3H).

### Example L & Example M

### Methyl 5-bromo-2-(bromomethyl)-3-(trifluoromethyl)benzoate (L) and 6-Bromo-4-(trifluoromethyl)isoindolin-1-one (M)

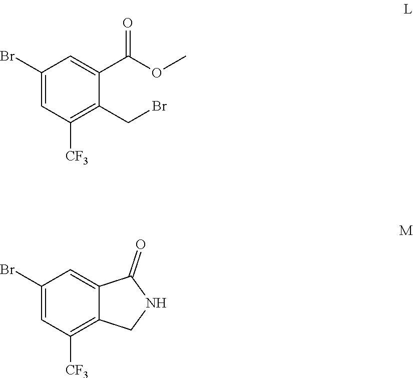 [SMILES: COC(=O)c1cc(Br)cc(C(F)(F)F)c1CBr ; O=C1NCc2c1cc(Br)cc2C(F)(F)F]

Step 1: Synthesis of methyl 5-bromo-2-methyl-3-(trifluoromethyl)benzoate. To a mixture of methyl 2-methyl-3-(trifluoromethyl)benzoate (15 g, 73.34 mmol) in acetic acid (100 mL) were added HNO₃ (46 g) and bromine (12.8 g, 80.10 mmol). Then AgNO₃ (16.1 g, 2.5M in water) was added to above mixture over 30 min. After stirred for 16 h at rt, The mixture was diluted with water. General Work-up Procedure 1 was followed. The residue was purified by Chromatography A to afford the title compound (14.0 g, 70%) as a colorless oil.

Step 2: Synthesis of methyl 5-bromo-2-(bromomethyl)-3-(trifluoromethyl)benzoate (L). A mixture of methyl 5-bromo-2-methyl-3-(trifluoromethyl)benzoate (14.0 g, 47.1 mmol), N-bromosuccinimide (NBS) (16.8 g, 94.4 mmol), benzoyl peroxide (BPO) (2.3 g, 8.9 mmol) in CCl₄ (150 mL) was stirred at 80° C. for 5 h. Then the solids were filtered off. The filtrate was concentrated. The residue was purified by Chromatography A to give the title compound (11.2 g, 63%) as a yellow oil.

Step 3: Synthesis of 6-bromo-4-(trifluoromethyl)isoindolin-1-one (M). To a stirred solution of methyl 5-bromo-2-(bromomethyl)-3-(trifluoromethyl)benzoate (11.2 g, 29.79 mmol) in THF (50 mL) was added NH₃ (7M in methanol, 50 mL). The mixture was stirred at rt for 16 h. The mixture was concentrated. The residue was diluted with water. General Work-up Procedure 1 was followed. The title compound (8.1 g, 53%) was obtained as a colorless solid using Chromatography A. MS (ESI) calc'd for (C₉H₅BrF₃NO) [M+H]⁺, 280.0; found, 280.1.

### Example N

### (R)-3-(1,1,2-trifluoro-1-(4-methyl-4H-1,2,4-triazol-3-yl)propan-2-yl)aniline (N)

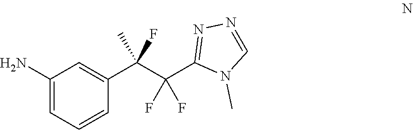 [SMILES: Cn1cnnc1C(F)(F)[C@](C)(F)c1cccc(N)c1]

Step 1: Synthesis of ethyl 3-(3-((tert-butoxycarbonyl)amino)phenyl)-2,2-difluoro-3-hydroxybutanoate. To a solution of Zn (231.9 g, 3.55 mol, 4.5 eq) and ethyl 2-bromo-2,2-difluoro-acetate (20.0 g, 98.5 mmol, 12.6 mL) in THF (1.6 L) was added DIBALH (1.00 M, 39.4 mL, 0.04 eq) at 30° C. Then the mixture was stirred at 30° C. for 1 h. Then tert-butyl (3-acetylphenyl)carbamate (200.0 g, 790.5 mmol, 29.2 mL) and ethyl 2-bromo-2,2-difluoro-acetate (200.0 g, 985.3 mmol, 126.5 mL) in THF (400 mL) was added dropwise at 40° C. and stirred at 40° C. for 3 h. Five batches were combined to work up. The mixture was filtered and the filtrate was poured into saturated NH₄Cl. Then extracted with EtOAc EtOAc (5 L*3). General Work-up Procedure 1 was followed to give the crude product which was purified by Chromatography A to give the title compound (700.0 g, 1.95 mol, 39.5% yield) as a yellow oil:

Step 2: Synthesis of tert-butyl (3-(3,3-difluoro-4-hydrazinyl-2-hydroxy-4-oxobutan-2-yl)phenyl)carbamate. To a solution of ethyl 3-(3-((tert-butoxycarbonyl)amino)phenyl)-2,2-difluoro-3-hydroxybutanoate (385.0 g, 1.07 mol) in EtOH (1.50 L) was added NH₂NH₂.H₂O (273.6 g, 5.36 mol, 265.6 mL, 98.0% purity). Then The mixture was stirred at 25° C. for 16 h. The mixture was concentrated to give the title compound (370.0 g, crude) which was used in the next step without purification.

Step 3: Synthesis of tert-butyl (3-(3,3-difluoro-2-hydroxy-4-(2-(methylcarbamothioyl)hydrazinyl)-4-oxobutan-2-yl)phenyl)carbamate. To a solution of tert-butyl (3-(3,3-difluoro-4-hydrazinyl-2-hydroxy-4-oxobutan-2-yl)phenyl)carbamate (370.0 g, 1.07 mol) in THF (1.50 L) was added methylimino(thioxo)methane (156.6 g, 2.14 mol, 146.4 mL). Then the mixture was stirred at 70° C. for 2 h. The mixture was concentrated to give the crude product (450.0 g, crude) as a yellow oil, which was used without purification

Step 4: Synthesis of tert-butyl (3-(1,1-difluoro-2-hydroxy-1-(5-mercapto-4-methyl-4H-1,2,4-triazol-3-yl)propan-2-yl)phenyl)carbamate. A solution of tert-butyl (3-(3,3-difluoro-2-hydroxy-4-(2-(methylcarbamothioyl)hydrazinyl)-4-oxobutan-2-yl)phenyl)carbamate (450.0 g, 1.08 mol) in NaOH (1.00 M, 4.50 L) was stirred at 50° C. for 2 h. The mixture was poured into HCl (0.50 M 1.5 L) and filtered. The filter cake was collected to give the crude product (430.0 g) as a colorless solid, which was used without purification

Step 5: Synthesis of tert-butyl (3-(1,1-difluoro-2-hydroxy-1-(4-methyl-4H-1,2,4-triazol-3-yl)propan-2-yl)phenyl)carbamate. To a solution of tert-butyl (3-(1,1-difluoro-2-hydroxy-1-(5-mercapto-4-methyl-4H-1,2,4-triazol-3-yl)propan-2-yl)phenyl)carbamate (214.0 g, 534.4 mmol, 1.0 eq) in methylene chloride (1.50 L) was added a solution of H₂O₂ (181.7 g, 1.60 mol, 154.0 mL, 30.0% purity) in AcOH (48.1 g, 801.6 mmol, 45.8 mL) drop-wise at 35° C. Then the reaction was stirred at 35° C. for 1 h. Two batches were combined to work up. The mixture was poured into saturated sodium carbonate and extracted with methylene chloride. The organic layers were combined and concentrated to give the crude product. The crude product was purified by Chromatography A to give the title compound (200 g, crude) as a colorless solid.

Step 6: Synthesis of tert-butyl (3-(1,1,2-trifluoro-1-(4-methyl-4H-1,2,4-triazol-3-yl)propan-2-yl)phenyl)carbamate. To a solution of compound 7 (115.0 g, 312.1 mmol) in methylene chloride (550 mL) was added DAST (150.9 g, 936.5 mmol, 123.7 mL). Then the mixture was stirred at 25° C. for 30 min. The mixture was poured into saturated NaHCO₃ and extracted with methylene chloride. The organic layers were combined and concentrated to give the crude product. The crude product was purified by Chromatography A to give the title compound (90.0 g, 39% yield) as a colorless solid.

Step 7: Synthesis of (R)-3-(1,1,2-trifluoro-1-(4-methyl-4H-1,2,4-triazol-3-yl)propan-2-yl)aniline. A solution of compound 7A (90.0 g, 241.7 mmol, 1.0 eq) in HCl/EtOAc EtOAc (4.00 M, 500.0 mL, 8.23 eq) was stirred at 25° C. for 16 h. The mixture was filtered. The filter cake was poured into saturated NaHCO₃ and General Work-up Procedure 1 was followed to give the racemic title compound (66.0 g, 231.0 mmol, 95.0% yield, 94.6% purity) as a yellow oil. The racemic product was separated by chiral SFC over a ChiralCel OJ column with EtOH in CO₂ to afford

(S)-3-(1,1,2-trifluoro-1-(4-methyl-4H-1,2,4-triazol-3-yl)propan-2-yl)aniline (26.4 g, colorless solid): 1H NMR (400 MHz, DMSO-d6) δ: 8.58 (s, 1H), 7.01 (t, J=8.0 Hz, 1H), 6.51-6.63 (m, 2H), 6.35 (d, J=8.0 Hz, 1H), 5.22 (s, 2H), 3.29 (s, 3H), 1.84 (d, J=24.0 Hz, 3H)

(R)-3-(1,1,2-trifluoro-1-(4-methyl-4H-1,2,4-triazol-3-yl)propan-2-yl)aniline (33.4 g, colorless solid): δ: 8.58 (s, 1H), 7.01 (t, J=8.0 Hz, 1H), 6.51-6.63 (m, 2H), 6.35 (d, J=8.0 Hz, 1H), 5.23 (br s, 2H), 3.29 (s, 3H), 1.84 (d, J=24.0 Hz, 3H).

### Example O

### (R)-3-(1-(1,3,4-thiadiazol-2-yl)propan-2-yl)aniline (O)

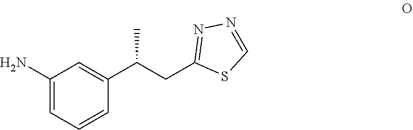

Step 1: Synthesis of (R)-3-(3-nitrophenyl)butanehydrazide. To a solution of ethyl (3R)-3-(3-nitrophenyl)butanoate (5.0 g, 21.1 mmol) in ethanol (10 mL) was added hydrazine hydrate (10 mL). The solution was stirred at 80° C. for 2 h before concentration under vacuum. The residue was then quenched by the addition of water (50 mL), and then extracted with dichloromethane (50 mL×6). The organic layers were combined, dried, and concentrated to afford the title compound (4.4 g, crude) as a colorless solid. The crude product was used without purification.

Step 2: Synthesis of (R)-N′-formyl-3-(3-nitrophenyl)butanehydrazide. A mixture of (3R)-3-(3-nitrophenyl)butanehydrazide (4.4 g, 19.71 mmol) in HCOOH (20 mL) was stirred at 25° C. for 16 h. When the reaction was completed, the mixture was then diluted with the addition of water (30 mL). The formed solids were collected by filtration to afford the title compound (4.9 g, crude) as a colorless solid, which was used without purification.

Step 3: Synthesis of (R)-2-(2-(3-nitrophenyl)propyl)-1,3,4-thiadiazole. To a solution of (3R)-N′formyl-3-(3-nitrophenyl)butanehydrazide (4.8 g, 19.1 mmol) in dioxane (80 mL) was added Lawesson's reagent (9.28 g, 22.94 mmol). The solution was stirred at 50° C. for 1 h. The reaction was then quenched by the addition of water (200 mL) and the aqueous phase was extracted with EtOAc (200 mL×3). General Work-up Procedure 1 was followed and the residue was purified by Chromatography A to afford the title compound (6.6 g, 72%) as a yellow oil.

Step 4: Synthesis of (R)-3-(1-(1,3,4-thiadiazol-2-yl)propan-2-yl)aniline. To a solution of 2-[(2R)-2-(3-nitrophenyl)propyl]-1,3,4-thiadiazole (2.7 g, 10.83 mmol) and NH₄Cl (3.48 g, 65.06 mmol) in ethanol (30 mL) and water (4 mL) was added Fe powder (3.64 g, 65.06 mmol). The solution was stirred at 90° C. for 16 h before concentration under vacuum. The residue was diluted with water (50 mL) and General Work-up Procedure 1 was followed. The crude residue was purified by Chromatography A to afford the title compound (1.9 g, 80%) as a light yellow oil. MS (ESI) calculated for (C₁₁H₃₁N₃S) [M+H]⁺, 220.1.1; found, 220.1.

### Example P1 and P2

### 3-((1R,2S)-2-(4-methyl-4H-1,2,4-triazol-3-yl)cyclopropyl)aniline (P1) and 3-((1S,2R)-2-(4-methyl-4H-1,2,4-triazol-3-yl)cyclopropyl)aniline (P2)

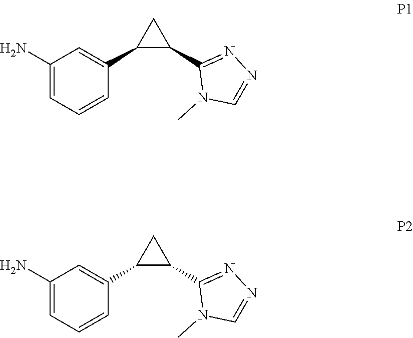 [SMILES: Cn1cnnc1[C@@H]1C[C@@H]1c1cccc(N)c1 ; Cn1cnnc1[C@H]1C[C@H]1c1cccc(N)c1]

Step 1: Synthesis of cis-2-(3-nitrophenyl)cyclopropanecarbohydrazide. To a solution of cis-ethyl 2-(3-nitrophenyl)cyclopropanecarboxylate (Valente, S. et. al., Eur. J. Org. Chem., 2015, 94, 163-174) (4.0 g, 17.0 mmol) in ethanol (40 mL) was added hydrazine hydrate (4 mL, 80%). The mixture was stirred at 80° C. for 48 h. The solvent was removed by vacuum to afford the title compound (3.0 g, crude) as a yellow solid, which was used without purification.

Step 2: Synthesis of (E)-N,N-dimethyl-N′-((cis)-2-(3-nitrophenyl)cyclopropane-1-carbonyl)formohydrazonamide. To a solution of cis-2-(3-nitrophenyl)cyclopropanecarbohydrazide (1.5 g, 6.8 mmol) in dichloromethane (50 mL) was added N,N-dimethylformamide dimethyl acetal (1.5 mL) and stirred at rt for 3 h. The solvent was removed under reduced pressure to afford the title compound (1.5 g, crude) as a yellow solid, which was used without purification.

Step 3: Synthesis of 4-methyl-3-(cis-2-(3-nitrophenyl)cyclopropyl)-4H-1,2,4-triazole. To a solution of (E)-N,N-dimethyl-N′-(cis-2-(3-nitrophenyl)cyclopropanecarbonyl)formohydrazonamide (500 mg, 1.81 mmol) in acetic acid (10 mL) was added methylamine (10 mL, 2 mol/L in THF). The mixture was stirred at 90° C. for 3 h before concentrated. The residue was diluted with water, and then alkalized with aqueous NaHCO₃ and General Work-up Procedure 1 was followed. The residue was purified by Chromatography B to afford the title compound (100 mg, 23%) as a yellow solid.

Step 4: Synthesis of 3-((1R,2S)-2-(4-methyl-4H-1,2,4-triazol-3-yl)cyclopropyl)aniline (P1) and 3-((1S,2R)-2-(4-methyl-4H-1,2,4-triazol-3-yl)cyclopropyl)aniline (P2). To a solution of 4-methyl-3-(cis-2-(3-nitrophenyl)cyclopropyl)-4H-1,2,4-triazole (600.0 mg, 2.46 mmol) in ethanol/water (10/5 mL) were added Fe powder (413.0 mg, 7.38 mmol) and NH₄Cl (652.0 mg, 12.20 mmol). The mixture was stirred at 70° C. for 3 h. The solids were filtered off. And the filtrate was evaporated under vacuum. The residue was purified by Chromatography B with to afford racemic title compound (300.0 mg, 57%) as a yellow solid. The racemic material (500.0 mg, 0.36 mmol) was separated by chiral-SFC conditions: [Column: Chiralpak AD-H, 2*25 cm (5 um); Mobile Phase A:CO2:80, Mobile Phase B: methanol-HPLC:20; Flow rate: 40 mL/min; 210 nm] to afford:

3-((1R,2S)-2-(4-methyl-4H-1,2,4-triazol-3-yl)cyclopropyl)aniline (P1, 130 mg, colorless solid): MS (ESI) calculated for (C₁₂H₁₄N₄) [M+H]⁺, 215.1; found, 215.0. ¹H NMR (300 MHz, CDCl₃) δ 7.86 (s, 1H), 6.92-6.87 (m, 1H), 6.42-6.39 (m, 2H) 6.38-6.16 (m, 1H) 3.33 (s, 3H), 2.49-2.41 (m, 1H), 2.28-2.21 (m, 1H), 2.06-2.00 (m, 1H), 1.66-1.59 (m, 1H).

3-((1S,2R)-2-(4-methyl-4H-1,2,4-triazol-3-yl)cyclopropyl)aniline (P2,140 mg, colorless solid): MS (ESI) calculated for (C₁₂H₁₄N₄) [M+H]⁺, 215.1; found, 215.0. ¹H NMR (300 MHz, CDCl₃) δ 7.85 (s, 1H), 6.92-6.86 (m, 1H), 6.42-6.37 (m, 2H) 6.35-6.16 (m, 1H) 3.32 (s, 3H), 2.49-2.41 (m, 1H), 2.28-2.20 (m, 1H), 2.06-2.00 (m, 1H), 1.66-1.58 (m, 1H).

### Example Q

### (R)-3-(2-(3-bromophenyl)propyl)-4-methyl-4H-1,2,4-triazole (Q)

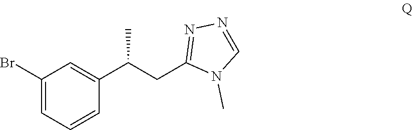 [SMILES: C[C@H](Cc1nncn1C)c1cccc(Br)c1]

Step 1: Synthesis of (4R)-3-[(2E)-3-(3-bromophenyl)prop-2-enoyl]-4-phenyl-1,3-oxazolidin-2-one. In a three necked bottle was placed a solution of (4R)-4-phenyl-1,3-oxazolidin-2-one (215.0 g, 1.32 mol) in anhydrous THF (1.0 L). Then LiHMDS (1.53 L, 1.53 mol, 1M in THF) was added dropwise to the above solution at −70° C. under nitrogen atmosphere. After stirring for 30 mins at −70° C., a solution of (E)-3-(3-bromophenyl)acryloyl chloride (Raffa, D., et. al. Eur. J. Med. Chem, 2013, 427-435)) in dry THF (1 L) was added dropwise to above mixture at −70° C. The mixture was stirred at −70˜−20° C. for 1 h. The reaction was quenched by saturated ammonium chloride aqueous solution at 0° C. and General Work-up Procedure 1 was followed. The residue was purified by trituration with petroleum ether/ethyl acetate (1/5) to afford the title compound (310 g, 33%) as a light yellow solid.

Step 2: Synthesis of (4R)-3-[(3R)-3-(3-bromophenyl)butanoyl]-4-phenyl-1,3-oxazolidin-2-one. To a suspension of CuBr.Me₂S (128.0 g, 0.62 mol) in anhydrous THF (1 L) was added MeMgBr (420 mL, 1.26 mol, 3 M in 2-methylTHF) dropwise with stirring at −40° C. under nitrogen atmosphere. Then the mixture was allowed to warm to −30˜−20° C. for 40 minutes. Then The mixture was cooled to ˜40° C., and to this mixture was added BF₃.Et₂O (88.7 g, 0.62 mol) dropwise with stirring at −40° C. Then the mixture was slowly warmed to −30˜−20° C. for another 40 minutes. Then the mixture was cooled to ˜40° C. again, to this was added a solution of (4R)-3-[(2E)-3-(3-bromophenyl)prop-2-enoyl]-4-phenyl-1,3-oxazolidin-2-one (155.0 g, 0.42 mol) in THF (0.5 L) slowly with stirring at −40˜−30° C. The mixture was allowed to warm to ˜−20° C. for 2 h. The reaction was then quenched by NH₄Cl (sat., aq.) and General Work-up Procedure 1 was followed. The residue was purified by recrystallization with methyl t-butyl ether and petroleum ether to afford (the title compound (100.0 g, 62%) as a colorless solid. MS (ESI) calculated for (C₁₉H₁₈BrNO₃) [M+H]⁺, 388.1; found, 387.9.

Step 3: Synthesis of (3R)-3-(3-bromophenyl)butanehydrazide. To a solution of (4R)-3-[(3R)-3-(3-bromophenyl)butanoyl]-4-phenyl-1,3-oxazolidin-2-one (77.0 g, 198.3 mmol) in THF (800 mL) was added NH₂NH₂.H₂O (33 mL, 80%) dropwise at 0° C. The mixture was stirred at rt for 16 h. The mixture was concentrated. The residue was diluted with water and General Work-up Procedure 1 was followed. The residue was purified by Chromatography A to afford the title compound (51.0 g, 99%) as a yellow oil.

Step 4: Synthesis of 5-[(2R)-2-(3-bromophenyl)propyl]-4-methyl-4H-1,2,4-triazole-3-thiol. To a solution of (3R)-3-(3-bromophenyl)butanehydrazide (46.0 g, 178.9 mmol) in THF (500 mL) was added isothiocyanatomethane (13.0 g, 177.8 mmol). The mixture was stirred at rt for 16 h. The mixture was concentrated. The residue was treated with sodium hydroxide (aq., 1M) and stirred at rt for 16 h. The mixture was acidified by HCl (2 N) to pH 3, then and General Work-up Procedure 1 was followed to afford the title compound (55.2 g, crude) as a yellow oil.

Step 5: Synthesis of 3-[(2R)-2-(3-bromophenyl)propyl]-4-methyl-4H-1,2,4-triazole. To a solution of 5-[(2R)-2-(3-bromophenyl)propyl]-4-methyl-4H-1,2,4-triazole-3-thiol (55.2 g, 176.79 mmol) in methylene chloride (600 mL) and acetic acid (300 mL) was added H₂O₂ (200 mL, 1.76 mol, 30% in water) dropwise with stirring at 0° C. The mixture was stirred at this temperature for 1 h before concentrated. The residue was dissolved in water and basified by NaOH (aq.) to pH 10, then and General Work-up Procedure 1 was followed. The residue was purified by Chromatography D with 0-10% methanol in EtOAc to afford the title compound (23.9 g, 48%) as a brown oil. MS (ESI) calculated for (C₁₂H₁₄BrN₃) [M+H]⁺, 280.0; found, 280.0. ¹H NMR (300 MHz, DMSO-d₆) δ 8.27 (s, 1H), 7.47 (d, J=1.8 Hz, 1H), 7.37 (d, J=7.2 Hz, 1H), 7.33-7.14 (m, 2H), 3.45 (s, 3H), 3.28-3.21 (m, 1H), 2.96 (d, J=7.5 Hz, 2H), 1.24 (d, J=6.9 Hz, 3H).

### Example R

### 3-(3-((4-methyl-4H-1,2,4-triazol-3-yl)methyl)oxetan-3-yl)aniline (R)

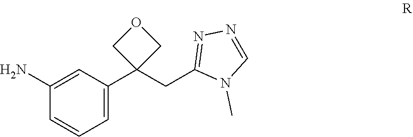 [SMILES: Cn1cnnc1CC1(c2cccc(N)c2)COC1]

Step 1: Synthesis of ethyl 2-(3-(3-nitrophenyl)oxetan-3-yl)acetate. Aqueous KOH (133.0 mL, 0.20 mol) was added to a suspension of [Rh(COD)CI]₂ (3.2 g, 6.5 mmol) in dioxane (100 ml) and the mixture was stirred for 30 min. Then 3-nitrophenylboronic acid (32.6 g, 0.20 mol) and ethyl 2-(oxetan-3-ylidene)acetate (WO2017107907) (18.6 g, 0.13 mol) in dioxane (40 mL) were added and The mixture was stirred at rt for 16 h under nitrogen. The reaction was quenched by the addition of HCl (1 N) to pH=6˜7. Then General Work-up Procedure 1 was followed. The residue was purified by Chromatography A to afford the title compound (25.6 g, 74%) as a yellow solid.

Step 2: Synthesis of 2-(3-(3-nitrophenyl)oxetan-3-yl)acetohydrazide. A mixture of ethyl 2-(3-(3-nitrophenyl)oxetan-3-yl)acetate (20.0 g, 75.4 mmol) in ethanol (100 mL) and hydrazine hydrate (20 mL) was stirred at 80° C. for 16 h. The solvent was removed under vacuum. The residue was triturated with EtOAc/petroleum ether (1/10) to afford the title compound (25.0 g, crude) as a yellow oil, which was used without purification.

Step 3: Synthesis of N-methyl-2-(2-(3-(3-nitrophenyl)oxetan-3-yl)acetyl)hydrazinecarbothioamide. To a solution of 2-(3-(3-nitrophenyl)oxetan-3-yl)acetohydrazide (10.0 g, 39.8 mmol) in THF (100 mL) was added isothiocyanatomethane (5.8 g, 19.1 mmol). The solution was stirred at rt for 4 h. The solvent was removed under vacuum. The residue was purified by Chromatography B to afford the title compound (10.0 g, 78%) as a yellow solid.

Step 4: Synthesis of 4-methyl-5-((3-(3-nitrophenyl)oxetan-3-yl)methyl)-4H-1,2,4-triazole-3-thiol. A mixture of N-methyl-2-(2-(3-(3-nitrophenyl)oxetan-3-yl)acetyl)hydrazine-carbothioamide (10.0 g, 30.8 mmol) in sodium hydroxide (308 mL, 1 M) was stirred at rt for 16 hours. The reaction was diluted with water. And then the pH value of the solution was adjusted to 5 with HCl (1 A). The solids were collected by filtration to afford the title compound (7.0 g) as a yellow solid, which was used without purification.

Step 5: Synthesis of 4-methyl-3-((3-(3-nitrophenyl)oxetan-3-yl)methyl)-4H-1,2,4-triazole. To a solution of 4-methyl-5-((3-(3-nitrophenyl)oxetan-3-yl)methyl)-4H-1,2,4-triazole-3-thiol (7.0 g, 22.8 mmol) in water (30 mL) was added NaNO₂ (15.8 g, 228.8 mmol). This was followed by the addition of HNO₃ (228.8 mL, 1 M) dropwise with stirring at 0° C. and stirred for another 1 h at 0° C. The mixture was basified by saturated NaHCO₃ aqueous and General Work-up Procedure 1 was followed to afford the title compound (6 g, crude) as a yellow solid, which was used without purification.

Step 6: Synthesis of 3-(3-((4-methyl-4H-1,2,4-triazol-3-yl)methyl)oxetan-3-yl)aniline. To a solution of 4-methyl-3-((3-(3-nitrophenyl)oxetan-3-yl)methyl)-4H-1,2,4-triazole (10 g, 36.5 mmol) in methanol (100 mL) was added Pd/C (dry, 4 g). The solution was stirred at rt for 16 h under hydrogen (2 atm). When the reaction was completed, the solids were filtered out. The filtrate was concentrated. The residue was purified by Chromatography C to afford the title compound (4.7 g, 53%) as a light yellow solid. MS (ESI) calculated for (C₁₃H₁₆N₄O) [M+H]⁺, 245.1; found, 245.0. ¹H NMR (300 MHz, DMSO-d₆) δ 8.19 (s, 1H), 6.92-6.87 (m, 1H), 6.40 (J=8.1 Hz, 1H), 6.05 (s, 1H), 5.94 (J=7.5 Hz, 1H), 5.00 (s, 2H), 4.90-4.84 (m, 2H), 4.79-4.74 (m, 2H), 3.38 (s, 2H), 2.83 (s, 3H).

### Example S

### 3-(2-(3-bromophenyl)-2-methylpropyl)-4-methyl-4H-1,2,4-triazole (S)

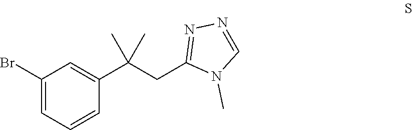 [SMILES: Cn1cnnc1CC(C)(C)c1cccc(Br)c1]

Step 1: Synthesis of methyl 3-(3-bromophenyl)-3-methylbutanoate. To a solution of 3-(3-bromophenyl)-3-methylbutanoic acid (WO2014144871) (7.0 g, 27.22 mmol) in methanol (100 mL) was added a solution of TMSCHN₂ in diethyl ether (20 mL, 2 M) at 0° C. The mixture was stirred at rt for 2 h. The mixture was evaporated in vacuo to afford methyl 3-(3-bromophenyl)-3-methylbutanoate (8.0 g, crude) as a brown oil.

Step 2: Synthesis of 3-(3-bromophenyl)-3-methylbutanehydrazide. To a solution of methyl 3-(3-bromophenyl)-3-methylbutanoate (8.0 g, crude) in ethanol (100 mL) was added hydrazine hydrate (8 mL, 85%). The mixture was stirred at 80° C. for 16 h. The reaction was evaporated in vacuo to afford the title compound (8.0 g, crude) as yellow oil, which was used without purification.

Step 3: Synthesis of 2-(3-(3-bromophenyl)-3-methylbutanoyl)-N-methylhydrazinecarbothioamide. To a solution of 3-(3-bromophenyl)-3-methylbutanehydrazide (2.0 g, crude) in THF (50 mL) was added isothiocyanatemethane (1.0 g, 14.75 mmol). The mixture was stirred at rt for 4 h, and then evaporated in vacuo. The residue was purified by Chromatography B afford the title compound (2.3 g, 91%) as a colorless solid.

Step 4: Synthesis of 5-(2-(3-bromophenyl)-2-methylpropyl)-4-methyl-4H-1,2,4-triazole-3-thiol. The solution of 2-(3-(3-bromophenyl)-3-methylbutanoyl)-N-methylhydrazinecarbothioamide (1.4 g, 4.07 mmol) in sodium hydroxide (40.7 mL, 1 M) was stirred at rt for 16 h. The mixture was diluted with water (50 mL). The pH value of the solution was adjusted to 5 with hydrochloric acid (1 M). The mixture was filtered. The solid was collected and dried to afford the title compound (1.20 g, crude) as a colorless solid.

Step 5: Synthesis of 3-(2-(3-bromophenyl)-2-methylpropyl)-4-methyl-4H-1,2,4-triazole. To a solution of 5-(2-(3-bromophenyl)-2-methylpropyl)-4-methyl-4H-1,2,4-triazole-3-thiol (1.4 g, 4.29 mmol) in dichloromethane/acetic acid (20/10 mL) was added hydrogen peroxide (1.5 mL). The mixture was stirred at 0° C. for 1 h. The mixture was concentrated. The residue was purified by Chromatography C w to afford the title compound (800 mg, 63%) as a yellow oil. MS (ESI) calculated for (C₁₃H₁₆BrN₃) [M+H]⁺, 294.2; found, 294.1.

### Example T

### 3-(2-methyl-1-(4-methyl-4H-1,2,4-triazol-3-yl)propan-2-yl)aniline (T)

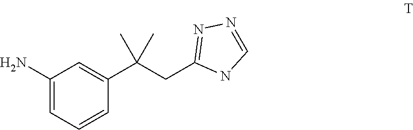 [SMILES: CC(C)(Cc1nnc[nH]1)c1cccc(N)c1]

Synthesis of 3-(2-methyl-1-(4-methyl-4H-1,2,4-triazol-3-yl)propan-2-yl)aniline. To a solution of 3-(2-(3-bromophenyl)-2-methylpropyl)-4-methyl-4H-1,2,4-triazole (800.0 mg, 2.72 mmol) in N-methyl-2-pyrrolidone (6 mL) was added NH₃.H₂O (6 mL) and Cu₂O (19.5 mg, 0.14 mmol). The mixture was stirred at 80° C. for 16 h. The mixture was cooled to rt. The mixture was purified by Chromatography C to afford the title compound (350 mg, 56%) as a yellow solid. MS (ESI) calculated for (C₁₃H₁₈N₄) [M+H]⁺, 231.2; found, 231.1.

### Example U

### 4-(bromomethyl)-3-(methoxycarbonyl)-5-(trifluoromethyl)benzoic acid (U)

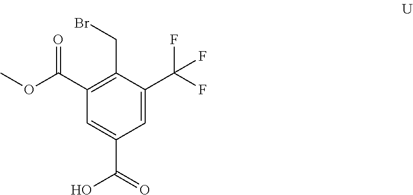 [SMILES: COC(=O)c1cc(C(=O)O)cc(C(F)(F)F)c1CBr]

Step 1: Synthesis of 3-(methoxycarbonyl)-4-methyl-5-(trifluoromethyl)benzoic acid. To a degassed solution of methyl 5-bromo-2-methyl-3-(trifluoromethyl)benzoate (18.0 g, 60.8 mmol), oxalic acid (11.5 g, 91.2 mmol), acetic anhydride (9.3 g, 91.2 mmol) and DIPEA (11.8 g, 91.2 mmol) in dimethylformamide (200 mL) were added Pd(OAc)₂ (1.4 g, 6.1 mmol) and XantPhos (1.8 g, 3.0 mmol). The mixture was stirred at 100° C. for 16 h under nitrogen. The reaction was quenched by the addition of HCl (1 M, 300 mL) to pH 3 and General Work-up Procedure 1 was followed. The residue was purified by Chromatography C to afford the title compound (7.5 g, 47%) as a light yellow solid.

Step 2: Synthesis of 4-(bromomethyl)-3-(methoxycarbonyl)-5-(trifluoromethyl)benzoic acid. To a stirred solution of 4-methyl-3-(methoxycarbonyl)-5-(trifluoromethyl)benzoic acid (8 g, 30.5 mmol) and NBS (8.2 g, 46 mmol) in CCl₄ (160 mL) was added benzoic peroxyanhydride (2.2 g, 9 mmol). The solution was stirred at 80° C. for 16 h. The mixture was concentrated. The crude product was purified by Chromatography B to afford the title compound (8.0 g, 77%) as a yellow solid. MS (ESI) calculated for (C₁₁H₈BrF₃O₄) [M−H]⁻, 339.0; found, 339.1 ¹H NMR (300 MHz, DMSO-d₆) δ 13.94 (s, 1H), 8.58 (d, J=1.8 Hz, 1H), 8.35 (d, J=1.8 Hz, 1H), 5.08 (s, 2H), 3.95 (s, 3H).

### Example V

### Methyl 2-(bromomethyl)-5-formyl-3-(trifluoromethyl)benzoate (V)

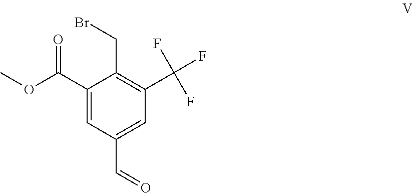 [SMILES: COC(=O)c1cc(C=O)cc(C(F)(F)F)c1CBr]

Step 1: Synthesis of methyl 2-(bromomethyl)-5-(hydroxymethyl)-3-(trifluoro-methyl)benzoate. To a stirred solution of 4-(bromomethyl)-3-(methoxycarbonyl)-5-(trifluoro-methyl)benzoic acid (2.65 g, 7.77 mmol) in THE (30 mL) was added borane (19.4 mL, 19.4 mmol, 1 M in THF). The solution was stirred at rt for 6 h. The reaction was then quenched by the addition of methanol (10 mL). The mixture was concentrated. The residue was purified by Chromatography A to afford the title compound (1.40 g, 84%) as a yellow oil.

Step 2: Synthesis of methyl 2-(bromomethyl)-5-formyl-3-(trifluoromethyl)-benzoate. To a stirred solution of methyl 2-(bromomethyl)-5-(hydroxymethyl)-3-(trifluoro-methyl)benzoate (7.1 g, 21.71 mmol) in EtOAc (70 mL) was added 2-iodoxybenzoic acid (9.1 g, 32.5 mmol). The reaction was stirred at 70° C. for 3 h. The solids was filtered, and the filtrate was concentrated in vacuo. The residue was purified by Chromatography A to afford the title compound (6.6 g, 94%) as a yellow oil. ¹H NMR (300 MHz, DMSO-d₆) δ 10.12 (s, 1H), 8.56 (d, J=1.8 Hz, 1H), 8.45 (d, J=1.8 Hz, 1H), 5.07 (s, 2H), 3.96 (s, 3H).

### Example W

### Methyl 5-acetyl-2-(bromomethyl)-3-(trifluoromethyl)benzoate (W)

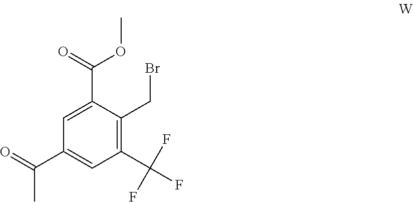 [SMILES: COC(=O)c1cc(C(C)=O)cc(C(F)(F)F)c1CBr]

Step 1: Synthesis of methyl 5-acetyl-2-methyl-3-(trifluoromethyl)benzoate. To a degassed solution of methyl 5-bromo-2-methyl-3-(trifluoromethyl)benzoate (20.0 g, 67.3 mmol) and tributyl(1-ethoxyvinyl)stannane (15.6 g, 67.5 mmol) in dioxane (300 mL) was added Pd(PPh₃)₂C₁₋₂ (3.8 g, 3.4 mmol). The mixture was stirred at 80° C. for 16 h under nitrogen. When the reaction was completed, the reaction was quenched by the addition of HCl (1N, 200 mL) and stirred for another 1 h. Then General Work-up Procedure 1 was followed. The residue was purified by Chromatography A to afford the title compound as a light yellow oil, which was used without purification.

Step 2: Synthesis of methyl 5-acetyl-2-(bromomethyl)-3-(trifluoromethyl)benzoate. To a mixture of 5-acetyl-2-methyl-3-(trifluoromethyl)benzoate (19.0 g, 73.1 mmol) and NBS (23.3 g, 131.5 mmol) in CCl₄ (300 mL) was added BPO (7.1 g, 29.2 mmol). The mixture was stirred at 80° C. for 24 h under nitrogen. When the reaction was completed, the solids were filtered. The filtrate was concentrated to give the crude product, which was purified by Chromatography D with 0˜50% dichloromethane in petroleum ether to afford the title compound (10.3 g, 42%) as a light yellow semi-solid. ¹H NMR (300 MHz, DMSO-d₆) δ 8.56 (d, J=1.8 Hz, 1H), 8.37 (d, J=1.8 Hz, 1H), 5.06 (s, 2H), 3.96 (s, 3H), 2.70 (s, 3H).

### Example X

### Methyl 3-(bromomethyl)-2-(trifluoromethyl)isonicotinate (X)

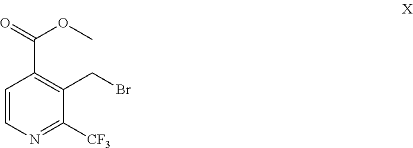 [SMILES: COC(=O)c1ccnc(C(F)(F)F)c1CBr]

Step 1: Synthesis of methyl 3-methyl-2-(trifluoromethyl)isonicotinate. A degassed solution of methyl 3-chloro-2-(trifluoromethyl)isonicotinate (WO2008076425) (11.6 g, 48.54 mmol), methylboronic acid (8.7 g, 145.6 mmol), K₃PO₄ (30.9 g, 145.6 mmol) and Pd(PCy₃)₂Cl₂ (1.8 g, 2.4 mmol) in toluene (170 mL) and water (17 mL) was stirred at 100° C. for 24 h under nitrogen. Then the reaction was quenched by the addition of 200 mL water and General Work-up Procedure 1 was followed. The residue was purified by Chromatography D with 0-20% ether in hexane to afford the title compound (7.1 g, 67%) as a yellow oil.

Step 2: Synthesis of methyl 3-(bromomethyl)-2-(trifluoromethyl)isonicotinate. To a solution of methyl 3-methyl-2-(trifluoromethyl)isonicotinate (7.1 g, 32.4 mmol) in CCl₄ (100 mL) were added NBS (6.3 g, 35.65 mmol) and BPO (1.6 g, 6.5 mmol). The mixture was refluxed at 80° C. for 16 h. When the reaction was complete, the reaction was quenched by the addition of 100 mL water. The aqueous solution was extracted with ether (100 mL×3) and General Work-up Procedure 1 was followed to give the residue, which was purified by Chromatography C to afford the title compound (5.1 g, 52%) as a yellow oil. MS (ESI) calculated for (C₉H₇BrF₃NO₂) [M+H]⁺, 298.0; found, 298.0. ¹H NMR (400 MHz, Chloroform-d) δ 8.76 (d, J=4.8 Hz, 1H), 7.89 (d, J=4.8 Hz, 1H), 5.07 (s, 2H), 4.05 (s, 3H).

### Example Y

### 6-bromo-2-(3-(3-((4-methyl-4H-1,2,4-triazol-3-yl)methyl)oxetan-3-yl)phenyl)-4-(trifluoro-methyl)isoindolin-1-one (Y)

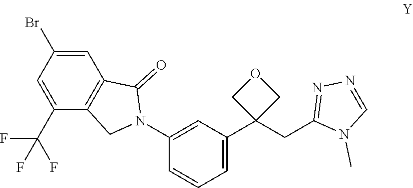 [SMILES: Cn1cnnc1CC1(c2cccc(N3Cc4c(cc(Br)cc4C(F)(F)F)C3=O)c2)COC1]

Step 1: Synthesis of methyl 5-bromo-2-methyl-3-(trifluoromethyl)benzoate. To a mixture of methyl 2-methyl-3-(trifluoromethyl)benzoate (15.0 g, 73.3 mmol) in acetic acid (100 mL) were added HNO₃ (46.0 g, 0.51 mol) and bromine (12.8 g, 80.1 mmol). And then to the above mixture was added AgNO₃ aqueous (16.1 g, 2.5M in water) slowly over ˜30 min at rt. After stirring for 16 h at rt, The mixture was diluted with water and General Work-up Procedure 1 was followed. The residue was purified by Chromatography A to afford the title compound (14.0 g, 70%) as a colorless oil.

Step 2: Synthesis of methyl 5-bromo-2-(bromomethyl)-3-(trifluoromethyl)benzoate. A mixture of methyl 5-bromo-2-methyl-3-(trifluoromethyl)benzoate (14.0 g, 47.1 mmol), NBS (16.8 g, 94.4 mmol), BPO (2.3 g, 8.9 mmol) in CCl₄ (150 mL) was stirred at 80° C. for 5 h. Then the solids were filtered. The filtrate was concentrated. The residue was purified by Chromatography to afford the title compound (11.2 g, 63%) as a yellow oil.

Step 3: Synthesis of 6-bromo-2-(3-(3-((4-methyl-4H-1,2,4-triazol-3-yl)methyl)oxetan-3-yl)phenyl)-4-(trifluoromethyl)isoindolin-1-one. To a degassed mixture of 3-(3-((4-methyl-4H-1,2,4-triazol-3-yl)methyl)oxetan-3-yl)aniline (2.1 g, 8.6 mmol) and methyl 5-bromo-2-(bromomethyl)-3-(trifluoromethyl)benzoate (3.5 g, 9.5 mmol) in ethanol (30 mL) was added TEA (1.7 g, 17.2 mmol). The mixture was stirred at 80° C. under nitrogen for 16 h. Then the mixture was filtered. The filtrate was concentrated to give the crude residue, which was purified by Chromatography to afford the title compound (1.65 g, 38%) as a yellow solid. MS (ESI) calculated for (C₂₂H₁₈BrF₃N₄O₂) [M+1]⁺, 507.1; found, 507.1. ¹H NMR (300 MHz, DMSO-d₆) δ 9.11 (s, 1H), 8.34-8.16 (m, 2H), 7.90-7.86 (m, 1H), 7.52 (t, J=2.1 Hz, 1H), 7.41 (t, J=8.1 Hz, 1H), 6.98-6.85 (m, 1H), 5.15 (s, 2H), 5.01-4.86 (m, 4H), 3.75 (s, 2H), 3.23 (s, 3H).

### Example Z

### 2-(3-(3-((4-methyl-4H-1,2,4-triazol-3-yl)methyl)oxetan-3-yl)phenyl)-3-oxo-7-(trifluoromethyl)isoindoline-5-carbaldehyde

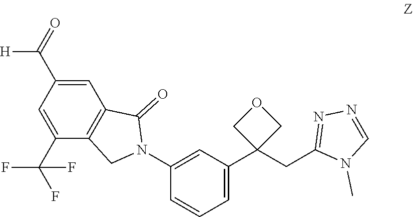 [SMILES: Cn1cnnc1CC1(c2cccc(N3Cc4c(cc(C=O)cc4C(F)(F)F)C3=O)c2)COC1]

Synthesis of 2-(3-{3-[(4-methyl-1,2,4-triazol-3-yl)methyl]oxetan-3-yl}phenyl)-3-oxo-7-(trifluoromethyl)-1H-isoindole-5-carbaldehyde: The indolone formation reaction was carried out analogously to 260, Step 2 by combining Example R (Example R) (11.3 g, 46.1 mmol) with methyl 2-(bromomethyl)-5-formyl-3-(trifluoromethyl)benzoate (Example V) (15.0 g, 46.1 mmol) in acetonitrile (410 mL) and water (205 mL). This was cooled to 0° C. before the addition of silver nitrate (10.2 g, 60.0 mmol) dissolved in 58 mL water. The reaction was stirred for 40 h at room temperature at which point solid sodium bicarbonate was added until solution was pH8. The mixture was then filtered through Celite, rinsing with acetonitrile (300 mL) followed by a DCM:ethyl acetate mixture (300 mL, 9:1). The organic layer was separated and dried over sodium sulfate. The crude residue was purified by Chromatography B. The oil obtained was azeotroped with toluene (3×150 mL) to afford the title compound (10.5 g, 50%) as a light-yellow solid. LCMS: C₂₃H₁₉F₃N₄O₃ requires: 456, found: m/z=557 [M+H]⁺.

### Example AA

### (R)-6-cyclopropyl-5-formyl-N-(3-(1-(4-methyl-4H-1,2,4-triazol-3-yl)propan-2-yl)phenyl)-picolinamide (AA)

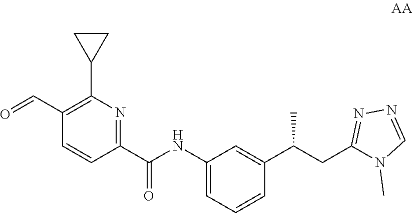 [SMILES: C[C@H](Cc1nncn1C)c1cccc(NC(=O)c2ccc(C=O)c(C3CC3)n2)c1]

Step 1: Synthesis of methyl 6-cyclopropyl-5-(hydroxymethyl)pyridine-2-carboxylate. A mixture of methyl 6-chloro-5-(hydroxymethyl)pyridine-2-carboxylate ((Gangadasu, B. et. al., Tetrahedron. 2006, 62, 8398-8403) (1.0 g, 5.0 mmol), potassium cyclopropyltrifluoroboranuide (2.1 g, 14.1 mmol), Pd(dppf)Cl₂ (770 mg, 1.05 mmol) and K₃PO₄ (3.8 g, 18.1 mmol) in toluene (40 mL) and water (4 mL) was heated to 100° C. for 16 h under nitrogen. The mixture was cooled to rt and then filtered. The filtrate was evaporated under vacuum. The residue was purified by Chromatography A to afford the title compound (834.0 mg, 81%) as a brown oil.

Step 2: Synthesis of 6-cyclopropyl-5-(hydroxymethyl)pyridine-2-carboxylic acid. A mixture of methyl 6-cyclopropyl-5-(hydroxymethyl)pyridine-2-carboxylate (170.0 mg, 0.82 mmol) and LiOH (45.0 mg, 1.88 mmol) in THF (6 mL) and water (2 mL) was stirred at rt for 3 h. The pH value of the mixture was adjusted to 5 with HCl (1N). The mixture was evaporated under vacuum to afford the title compound (200.0 mg, crude) as a colorless solid, which was used without purification.

Step 3: Synthesis of 6-cyclopropyl-5-(hydroxymethyl)-N-[3-[(2R)-1-(4-methyl-4H-1,2,4-triazol-3-yl)propan-2-yl]phenyl]pyridine-2-carboxamide. To a mixture of 6-cyclopropyl-5-(hydroxymethyl)pyridine-2-carboxylic acid (200.0 mg, crude) in DMF (3 mL) were added DIPEA (1 mL, 6.05 mmol), 3-[(2R)-1-(4-methyl-4H-1,2,4-triazol-3-yl)propan-2-yl]aniline (173.6 mg, 0.80 mmol) and HATU (883.0 mg, 2.32 mmol). The mixture was stirred at rt for 2 h. The mixture was concentrated and purified by Chromatography C and then purified by Prep-HPLC to afford the title compound (31.6 mg, 10%) as an off-white solid.

Step 4: Synthesis of (R)-6-cyclopropyl-5-formyl-N-(3-(1-(4-methyl-4H-1,2,4-triazol-3-yl)propan-2-yl)phenyl)picolinamide. To a solution of (R)-6-cyclopropyl-5-(hydroxymethyl)-N-(3-(1-(4-methyl-4H-1,2,4-triazol-3-yl)propan-2-yl)phenyl)picolinamide (3.1 g, 7.9 mmol) in methylene chloride (30 mL) was added Dess-Martin reagent (4.0 g, 9.5 mmol) at 0° C. The mixture was stirred at 0° C. for 1 h, and then quenched by the addition of aqueous saturated NaHCO₃ and General Work-up Procedure 1 was followed. The crude residue was purified by Chromatography to afford the title compound (1.8 g, 58%) as a light brown foam. MS (ESI) calculated for (C₂₂N₂₃N₅O₂) [M+H]⁺, 390.2; found 390.2. ¹H NMR (300 MHz, DMSO-d₆) δ 10.51 (s, 1H), 10.10 (s, 1H), 8.36 (d, J=8.1 Hz, 1H), 8.29 (s, 1H), 8.04 (d, J=8.1 Hz, 1H), 7.73-7.68 (m, 2H), 7.38-7.25 (m, 1H), 7.12-7.01 (m, 1H), 3.47 (s, 3H), 3.34-3.24 (m, 1H), 3.22-3.12 (m, 1H), 2.99 (d, J=7.5 Hz, 2H), 1.49-1.44 (m, 2H), 1.30 (d, J=6.9 Hz, 3H), 1.17-1.12 (m, 2H).

### Example AB

### (R)-2-(6-chloro-4-(1-(4-methyl-4H-1,2,4-triazol-3-yl)propan-2-yl)pyridin-2-yl)-4-(trifluoromethyl)isoindolin-1-one (AB)

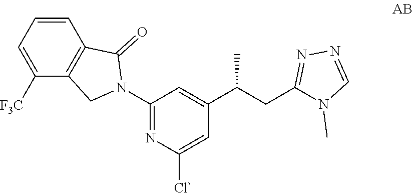

Step 1: Synthesis of (E)-3-(2,6-dichloropyridin-4-yl)acrylic acid. To a solution of (E)-ethyl 3-(2,6-dichloropyridin-4-yl)acrylate (WO2012103008) (121.5 g, 20.8 mmol) in THF (1 L) and water (1 L) was added LiOH (23.8 g, 993.8 mmol) in portions at 0° C. The mixture was stirred at rt for 3 h. The solvents were removed under vacuum. The residue was diluted with water and the aqueous phase was extracted with EtOAc. The aqueous layer was adjusted to pH3 by HCl (1 N) and General Work-up Procedure 1 was followed to afford the title compound as an off-white solid (106.8 g, crude), which was used without purification.

Step 2: Synthesis of (R,E)-3-(3-(2,6-dichloropyridin-4-yl)acryloyl)-4-phenyloxazolidin-2-one. To a solution of (E)-3-(2,6-dichloropyridin-4-yl)acrylic acid (106.8 g, 0.49 mol) and trithylamine (108.8 g, 1.08 mol) in THF (1.5 L) was added pivaloyl chloride (58.8 g, 0.49 mol) dropwise at −15° C. The mixture was stirred at −15° C. for 1 h. LiCl (20.8 g, 0.49 mol) and a solution of (R)-4-phenyloxazolidin-2-one (79.9 g, 0.49 mol) in THF (300 mL) was added. The mixture was stirred at rt for 16 h. The reaction was then quenched by addition of saturated NH₄Cl aqueous and General Work-up Procedure 1 was followed. The residue was purified by Chromatography A to afford (the title compound (136.8 g, 77%) as a colorless solid.

Step 3: Synthesis of (R)-3-((R)-3-(2,6-dichloropyridin-4-yl)butanoyl)-4-phenyloxazolidin-2-one. To a suspension of CuBrMe₂S (7.5 g, 36.78 mmol) in anhydrous THF (90 mL) was added MeMgBr (25.4 mL, 76.2 mmol, 3 M in 2-methylTHF) at −40° C. under nitrogen atmosphere. Then the mixture was allowed to warm to −40˜−20° C. for 40 minutes. Then the mixture was cooled to ˜40° C., and to this mixture was added BF₃.Et₂O (5.2 g, 36.78 mmol) dropwise with stirring at −40° C. Then the mixture was slowly warmed to ˜−40 ˜−20° C. for another 40 min. Then the mixture was cooled to ˜40° C. again, to this was added a solution of (R,E)-3-(3-(2,6-dichloropyridin-4-yl)acryloyl)-4-phenyloxazolidin-2-one (8.9 g, 24.5 mmol) slowly with stirring at −40˜−30° C. The mixture was allowed to warm to ˜−20° C. for 2 h. The reaction was then quenched by the addition of saturated ammonium chloride solution and General Work-up Procedure 1 was followed. The resulting residue was purified by Chromatography A to afford the title compound (3.7 g, 39%) as an off-white syrup.

Step 4: Synthesis of (R)-3-(2,6-dichloropyridin-4-yl)butanehydrazide. To a solution of (R)-3-((R)-3-(2,6-dichloropyridin-4-yl)butanoyl)-4-phenyloxazolidin-2-one (2.7 g, 7.29 mmol) in THF (20 mL) was added hydrazine hydrate (730 mg, 14.58 mmol). The solution was stirred at rt for 16 h. The mixture was concentrated to afford the title compound (1.76 g, crude) as a yellow oil, which was used without purification.

Step 5: Synthesis of (R)-5-(2-(2,6-dichloropyridin-4-yl)propyl)-4-methyl-4H-1,2,4-triazole-3-thiol. To a solution of (R)-3-(2,6-dichloropyridin-4-yl)butanehydrazide (740 mg, 2.98 mmol) in THF (8 mL) was added isothiocyanatomethane (260 mg, 3.56 mmol). The solution was stirred at rt for 4 h. A solution of NaOH (1 N, 8 mL) was added. The mixture was stirred at rt for 2 h before diluted with water (10 mL). The pH of the mixture was adjusted to 3 by HCl (1 N), and General Work-up Procedure 1 was followed. The residue was purified by Chromatography to afford the title compound (380 mg, 42%) as a yellow oil.

Step 6: Synthesis of (R)-2,6-dichloro-4-(1-(4-methyl-4H-1,2,4-triazol-3-yl)propan-2-yl)pyridine. To a solution of (R)-5-(2-(2,6-dichloropyridin-4-yl)propyl)-4-methyl-4H-1,2,4-triazole-3-thiol (386 mg, 1.27 mmol) in AcOH (2 mL) and dichloromethane (4 mL) was added H₂O₂ (430 mg, 3.82 mmol, 30%) dropwise at 0° C. The mixture was stirred at rt for 2 h. The reaction was then diluted with water and General Work-up Procedure 1 was followed. The residue was purified by Chromatography B to afford the title compound (100 mg, 28%) as a yellow oil.

Step 7: Synthesis of (R)-2-(6-chloro-4-(1-(4-methyl-4H-1,2,4-triazol-3-yl)propan-2-yl)pyridin-2-yl)-4-(trifluoromethyl)isoindolin-1-one (AB). A degassed solution of (R)-2,6-dichloro-4-(1-(4-methyl-4H-1,2,4-triazol-3-yl)propan-2-yl)pyridine (6.9 g, 25.6 mmol), 4-(trifluoromethyl)isoindolin-1-one (5.2 g, 25.9 mmol), XantPhos (1.5 g, 2.6 mmol), K₃PO₄(11.1 g, 52.4 mmol) and Pd(AcO)₂ (0.6 g, 2.7 mmol) in dioxane (70 mL) was stirred at 100° C. for 16 h under nitrogen. The reaction was quenched by the addition of water and General Work-up Procedure 1 was followed. The resulting residue was purified by Chromatography C to afford the title compound (5.0 g, 45%) as an off-white solid. MS (ESI) calculated for (C₂₀H₁₇ClF₃N₅O) [M+H]⁺, 436.1; found 436.1. ¹H NMR (400 MHz, Chloroform-d) δ 8.49 (d, J=1.2 Hz, 1H), 8.10 (d, J=7.6 Hz, 1H), 8.03 (s, 1H), 7.89 (d, J=7.6 Hz, 1H), 7.72-7.64 (m, 1H), 7.02 (d, J=1.2 Hz, 1H), 5.39-5.11 (m, 2H), 3.58 (s, 3H), 3.58-3.55 (m, 1H), 3.13-2.98 (m, 2H), 1.45 (d, J=6.8 Hz, 3H).

### Example AC

### (S)-2-((3-((3-(1-((4-methyl-4H-1,2,4-triazol-3-yl)thio)ethyl)phenyl)carbamoyl)isoquinolin-7-yl)oxy)acetic acid (AC)

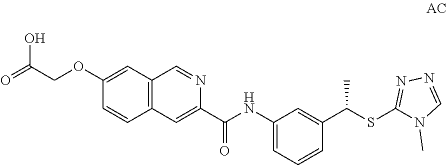 [SMILES: C[C@H](Sc1nncn1C)c1cccc(NC(=O)c2cc3ccc(OCC(=O)O)cc3cn2)c1]

Step 1: Synthesis of 7-hydroxyisoquinoline-3-carboxylic acid. A mixture of methyl 7-hydroxyisoquinoline-3-carboxylate (WO2005082858) (1.6 g, 7.9 mmol) and LiOH (750.0 mg, 31.32 mmol) in THF (30 mF) and water (15 mF) was stirred at rt for 16 h. The pH was adjusted to 4 with HCl (1 N). The mixture was evaporated under vacuum to afford the title compound (1.5 g, crude) as a yellow solid, which was used without purification.

Step 2: Synthesis of (S)-7-hydroxy-N-(3-(1-(4-methyl-4H-1,2,4-triazol-3-ylthio)ethyl)phenyl)isoquinoline-3-carboxamide. To a solution of 7-hydroxyisoquinoline-3-carboxylic acid (1.5 g, crude) in DMF (30 mF) were added (S)-3-(1-(4-methyl-4H-1,2,4-triazol-3-ylthio)ethyl)aniline (1.9 g, 8.12 mmol), HATU (5.5 g, 14.47 mmol) and DIPEA (4.6 g, 35.65 mmol) at 0° C. under N₂. The mixture was stirred at 0° C. for 3 h. The mixture was diluted with water and General Work-up Procedure 1 was followed. The residue was purified by Chromatography B to afford the title compound (1.1 g, 24%) as a yellow solid, which was used without purification.

Step 3: Synthesis of tert-butyl 2-[[3-([3-[(1S)-1-[(4-methyl-4H-1,2,4-triazol-3-yl)sulfanyl]ethyl]phenyl]carbamoyl)isoquinolin-7-yl]oxy]acetate. To a solution of (S)-7-hydroxy-N-(3-(1-(4-methyl-4H-1,2,4-triazol-3-ylthio)ethyl)phenyl)isoquinoline-3-carboxamide (1.0 g, 2.5 mmol) in DMF (10 mF) were added potassium carbonate (524 mg, 3.79 mmol) and tert-butyl 2-bromoacetate (481 mg, 2.47 mmol). The mixture was stirred at 60° C. for 30 min. The mixture was cooled to rt and then filtered. The filtrate was diluted with EtOAc, washed with brine, dried, and filtered. The filtrate was evaporated under vacuum. The residue was purified by Chromatography B to afford the title compound (700 mg, 55%) as a colorless solid.

Step 4: Synthesis of (S)-2-((3-((3-(1-((4-methyl-4H-1,2,4-triazol-3-yl)thio)ethyl)phenyl)carbamoyl)isoquinolin-7-yl)oxy)acetic acid. A mixture of tert-butyl 2-[[3-([3-[(1S)-1-[(4-methyl-4H-1,2,4-triazol-3-yl)sulfanyl]ethyl]phenyl]carbamoyl)isoquinolin-7-yl]oxy]acetate (200 mg, 0.38 mmol) in HCl/1,4-dioxane (10 mL, 4 mol/L) was stirred at 0° C. for 1 h. The mixture was concentrated. The pH value of the mixture was adjusted to 5 with HCl (1 N). The mixture was evaporated under vacuum. The residue was purified by Prep-HPLC to afford the title compound (49 mg, 27%) as a colorless solid. MS (ESI) calculated for (C₂₃H₂₁N₅O₄S) [M+H]⁺, 464.1; found, 464.2. ¹H NMR (400 MHz, DMSO-d₆) δ13.30 (s, 1H), 10.70 (s, 1H), 9.35 (s, 1H), 8.64 (s, 1H), 8.55 (s, 1H), 8.20 (d, J=9.2 Hz, 1H), 7.99 (s, 1H), 7.91-7.89 (m, 1H), 7.68 (d, J=2.4 Hz, 1H), 7.62-7.59 (m, 1H), 7.33-7.29 (m, 1H), 7.03 (d, J=7.6 Hz, 1H), 4.91 (s, 2H), 4.71-4.65 (m, 1H), 3.40 (s, 3H), 1.68 (d, J=6.8 Hz, 3H).

### Example AD

### 3-[3-[(4-methyl-1,2-oxazol-3-yl)methyl]oxetan-3-yl]aniline (AD)

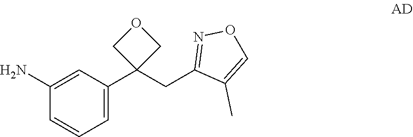 [SMILES: Cc1conc1CC1(c2cccc(N)c2)COC1]

Step 1: Synthesis of 2-(3-(3-bromophenyl)oxetan-3-yl)ethan-1-ol. To a solution of ethyl 2-[3-(3-bromophenyl)oxetan-3-yl]acetate (10.0 g, 33.4 mmol) in anhydrous THF (100 mL) was added LiAlH₄ (1.3 g, 33.4 mmol) in portions at 0° C. The mixture was stirred at 0° C. for 3 h. The reaction was quenched by the addition of water at 0° C. slowly followed by General Work-up Procedure 1. The residue was purified by Chromatography A to afford the title compound (6.3 g, 73%) as a colorless oil.

Step 2: Synthesis of 2-(3-(3-bromophenyl)oxetan-3-yl)acetaldehyde. To a solution of 2-(3-(3-bromophenyl)oxetan-3-yl)ethan-1-ol (6.3 g, 24.50 mmol) in DCM (70 mL) was added Dess-Martin reagent (15.6 g, 36.8 mmol). The mixture was stirred at rt for 2 h. The reaction was quenched by saturated NaHCO₃ aqueous solution followed by General Work-up Procedure 1. The residue was purified by Chromatography A to afford the title compound (6.0 g, 95%) as a colorless oil.

Step 3: Synthesis of (E)-2-(3-(3-bromophenyl)oxetan-3-yl)acetaldehyde oxime. To a solution of 2-(3-(3-bromophenyl)oxetan-3-yl)acetaldehyde (6.0 g, 23.5 mmol) in DCM (50 mL) were added hydroxylamine hydrochloride (2.45 g, 35.3 mmol), and TEA (3.56 g, 35.3 mmol). The solution was stirred at rt for 20 h. The reaction was then quenched by the addition of water and extracted with dichloromethane followed by General Work-up Procedure 1. The residue was purified by chromatography A to afford the title compound (5.38 g, 85%) as a colorless oil.

Step 4: Synthesis of (Z)-2-(3-(3-bromophenyl)oxetan-3-yl)-N-hydroxyacetimidoyl chloride. To a solution of (E)-2-(3-(3-bromophenyl)oxetan-3-yl)acetaldehyde oxime (5.38 g, 19.92 mmol) in DMF (50 mL) was added NCS (2.93 g, 21.91 mmol). The solution was stirred at rt for 1 h. The reaction was then quenched by the addition of water followed by General Work-up Procedure 1 to afford the title compound (6.0 g) as a colorless oil, which was used in the next step without purification.

Step 5: Synthesis of ethyl 3-[[3-(3-bromophenyl)oxetan-3-yl]methyl]-1,2-oxazole-4-carboxylate. To a solution of (Z)-2-(3-(3-bromophenyl)oxetan-3-yl)-N-hydroxyacetimidoyl chloride (6.0 g, 19.70 mmol) in CHCl₃ (50 mL) were added ethyl (2E)-3-(dimethylamino)prop-2-enoate (4.23 g, 29.55 mmol) and TEA (2.99 g, 29.55 mmol). The solution was stirred at rt for 16 h. The mixture was then quenched by the addition of water and extracted with DCM and followed by General Work-up Procedure 1. The residue was purified by chromatography A to afford the title compound (2.88 g, 40%) as a yellow oil.

Step 6: Synthesis of (3-[[3-(3-bromophenyl)oxetan-3-yl]methyl]-1,2-oxazol-4-yl)methanol. To a solution of ethyl 3-[[3-(3-bromophenyl)oxetan-3-yl]methyl]-1,2-oxazole-4-carboxylate (2.88 g, 7.89 mmol) in anhydrous THF (40 mL) was added LiAlH₄ (360 mg, 9.47 mmol) at −70° C. The solution was stirred at 0° C. for 2 h. The mixture was then quenched by the addition of water followed by General Work-up Procedure 1. The residue was by chromatography A to afford the title compound (1.4 g, 55%) as a colorless oil.

Step 7: Synthesis of (3-((3-(3-bromophenyl)oxetan-3-yl)methyl)isoxazol-4-yl)methyl methanesulfonate. To a solution of (3-[[3-(3-bromophenyl)oxetan-3-yl]methyl]-1,2-oxazol-4-yl)methanol (600 mg, 1.86 mmol) in DCM (10 mL) were added TEA (375 mg, 3.71 mmol) and methanesulfonyl chloride (318 mg, 2.79 mmol). The solution was stirred at rt for 2 h. The mixture was then quenched by the addition of water and extracted with dichloromethane followed by General Work-up Procedure 1 to afford the title compound (610 mg) as a yellow oil, which was used in the next step without purification.

Step 8: Synthesis of 3-[[3-(3-bromophenyl)oxetan-3-yl]methyl]-4-methyl-1,2-oxazole. To a solution of (3-((3-(3-bromophenyl)oxetan-3-yl)methyl)isoxazol-4-yl)methylmethanesulfonate (800 mg, 1.99 mmol) in DMSO (20 mL) was added NaBH₄ (150 mg, 3.978 mmol). The solution was stirred at 60° C. for 1 h. The mixture was then quenched by the addition of water followed by General Work-up Procedure 1. The residue was purified by chromatography A to afford the title compound (350 mg, 56%) as an off-white solid.

Step 9: Synthesis of 3-[3-[(4-methyl-1,2-oxazol-3-yl)methyl]oxetan-3-yl]aniline. To a degassed solution of 3-[[3-(3-bromophenyl)oxetan-3-yl]methyl]-4-methyl-1,2-oxazole (500 mg, 1.62 mmol) in MeCN (10 mL) and ammonia (10 mL) was added Cu₂O (46 mg, 0.32 mmol). The solution was stirred at 90° C. for 12 h in a sealed tube under nitrogen. The solids were removed by filtration. The filtrate was concentrated. The residue was purified by chromatography C to afford the title compound (267.3 mg, 67%) as an off-white solid. LCMS: C₁₄H₁₆N₂O₂ requires: 244, found: m/z=245 [M+H]⁺.

### Example AE

### 3-[3-[(4-methyl-1H-pyrazol-3-yl)methyl] oxetan-3-yl] aniline (AE)

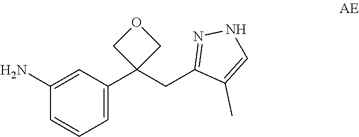 [SMILES: Cc1c[nH]nc1CC1(c2cccc(N)c2)COC1]

Step 1: Synthesis of 2-(3-(3-nitrophenyl)oxetan-3-yl)acetic acid. To a suspension of ethyl 2-[3-(3-nitrophenyl)oxetan-3-yl]acetate (10.0 g, 37.70 mmol) in THF (100 mL) and water (100 mL) was added NaOH (3.0 g, 75.40 mmol). The mixture was stirred at rt for 3 h. The organic solvent was removed under vacuum and the aqueous solution was acidified to pH 34 by HCl (1 N) followed by General Work-up Procedure 1 to afford the title compound (8.1 g, 91%) as an off-white solid.

Step 2: Synthesis of N-methoxy-N-methyl-2-(3-(3-nitrophenyl)oxetan-3-yl)acetamide. A mixture of 2-[3-(3-nitrophenyl)oxetan-3-yl]acetic acid (8.0 g, 33.73 mmol), methoxy(methyl)amine hydrochloride (3.9, 40.13 mmol), HATU (19.0 g, 49.91 mmol) and DIPEA (13.0 g, 100.50 mmol) in DMF (100 mL) was stirred at rt for 16 h. The reaction was then quenched by the addition of water followed by General Work-up Procedure 1. The residue was purified by chromatography A to afford the title compound (9.0 g, 95%) as a white solid.

Step 3: Synthesis of 2-[3-(3-aminophenyl)oxetan-3-yl]-N-methoxy-N-methylacetamide. To a mixture of N-methoxy-N-methyl-2-[3-(3-nitrophenyl)oxetan-3-yl]acetamide (9.0 g, 32.11 mmol) in MeOH (250 mL) was added Pd/C (10%, 0.9 g, 8.36 mmol). The solution was stirred at rt for 16 h under hydrogen (2 atm). The solids were removed by filtration. The filtrate was concentrated under vacuum to afford the title compound (7.0 g) as a white solid, which was used in the next step without purification.

Step 4: Synthesis of benzyl N-[3-(3-[[methoxy(methyl)carbamoyl]methyl]oxetan-3-yl)phenyl]carbamate. To a mixture of 2-[3-(3-aminophenyl)oxetan-3-yl]-N-methoxy-N-methylacetamide (2.0 g, 7.99 mmol), in DCM (50 mL) were added benzyl chloroformate (2.7 g, 16.00 mmol) and DIPEA (4.1 g, 24.00 mmol) was stirred at rt for 24 h. The mixture was then quenched by the addition of water followed by General Work-up Procedure 1. The residue was purified by chromatography A to afford the title compound (1.8 g, 57%) as a white solid.

Step 5: Synthesis of benzyl N-[3-[3-(2-oxobutyl)oxetan-3-yl]phenyl]carbamate. To a stirred solution of benzyl N-[3-(3-[[methoxy(methyl)carbamoyl]methyl]oxetan-3-yl)phenyl]carbamate (1.8 g, 4.60 mmol) in anhydrous THF (30 mL) was added ethylmagnesium bromide (5.4 mL, 2 M in Et₂O) dropwise at −70° C. under N₂ atmosphere. The mixture was stirred at rt for 16 h. The reaction was then quenched by the addition of saturated NH₄Cl aqueous solution followed by General Work-up Procedure 1. The residue was purified by chromatography A to afford the title compound (800 mg, 49%) as a colorless syrup.

Step 6: Synthesis of benzyl N-(3-[3-[(3E)-4-(dimethylamino)-3-methyl-2-oxobut-3-en-1-yl]oxetan-3-yl]phenyl)carbamate. A mixture of benzyl N-[3-[3-(2-oxobutyl)oxetan-3-yl]phenyl]carbamate (5.0 g, 14.15 mmol) and [bis(tert-butoxy)methyl]dimethylamine (8.63 g, 42.25 mmol) in toluene (50 mL) was stirred at reflux for 16 h. The solvent was removed under vacuum to afford the title compound (8.0 g) as red oil. The product was used in next step without purification.

Step 7: Synthesis of benzyl N-(3-[3-[(4-methyl-1H-pyrazol-3-yl)methyl]oxetan-3-yl]phenyl)carbamate. A mixture of benzyl N-(3-[3-[(3E)-4-(dimethylamino)-3-methyl-2-oxobut-3-en-1-yl]oxetan-3-yl]phenyl)carbamate (408 mg) and hydrazine hydrate (375 mg, 80%) in EtOH (4 mL) was stirred at 80° C. for 16 h. The solvents were removed under vacuum and the residue was purified by chromatography C to afford the title compound (130 mg, 12% over two steps) as a colorless solid.

Step 8: Synthesis of 3-[3-[(4-methyl-1H-pyrazol-3-yl)methyl]oxetan-3-yl]aniline. To a mixture of benzyl N-(3-[3-[(4-methyl-1H-pyrazol-3-yl)methyl]oxetan-3-yl]phenyl)carbamate (120 mg, 0.32 mmol) in EtOH (5 mL) was added Pd/C (10%, 30 mg) was stirred at rt for 2 h under H₂ (2 atm). After filtration, the filtrate was concentrated under vacuum and the residue was purified by chromatography C to afford the title compound (30 mg, 39%) as a colorless solid. LCMS: C₁₄H₁₇N₃O requires: 243, found: m/z=244 [M+H]⁺.

### Example AF

### 3-((3-(3-bromophenyl)oxetan-3-yl)fluoromethyl)-4-methyl-4H-1,2,4-triazole (AF)

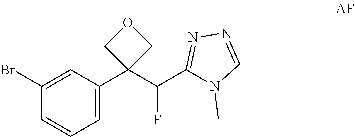 [SMILES: Cn1cnnc1C(F)C1(c2cccc(Br)c2)COC1]

Step 1: Synthesis of ethyl 2-[3-(3-bromophenyl)oxetan-3-yl]acetate. To a stirred solution of [Rh(COD)CI]₂ (55.5 g, 112 mmol) in dioxane (1 L) were added aqueous KOH (844 mL, 1.27 mol) dropwise at 0° C. under nitrogen atmosphere. The mixture was stirred for 30 min at 0° C. To the above mixture was added ethyl 2-(oxetan-3-ylidene)acetate (320 g, 2.25 mol) in portions. Then a solution of (3-bromophenyl)boronic acid (678 g, 3.38 mol) in dioxane (3.2 L) was dropwise into the above mixture in 1 h. The mixture was stirred for 30 min at rt. An additional amount of 3-bromophenyl)boronic acid (226 g, 1.13 mol) was added. The mixture was stirred for 12 h at rt. The mixture was diluted with brine and followed by General Work-up Procedure 1. The residue was purified by chromatography A to afford the title compound (405 g, 60%) as a yellow oil.

Step 2: Synthesis of ethyl 2-[3-(3-bromophenyl)oxetan-3-yl]-2-hydroxyacetate. To a stirring mixture of ethyl 2-[3-(3-bromophenyl)oxetan-3-yl]acetate (101.5 g, 339.2 mmol) in THF (1.2 L) were added KHMDS (509 mL, 508.9 mmol, 1M in THF) dropwise at −78° C. under nitrogen atmosphere. The mixture was stirred for 40 min. To the above mixture were added 2-(benzenesulfonyl)-3-phenyloxaziridine (115 g, 441 mmol) in THF (400 mL) dropwise at −65° C. The mixture was stirred for 3 h at −65° C. Then the reaction was warmed to rt. The reaction was quenched with aqueous NH₄Cl followed by General Work-up Procedure 1. The residue was purified by chromatography A to afford the title compound (81 g, 76%) as a white solid.

Step 3: Synthesis of 2-[3-(3-bromophenyl)oxetan-3-yl]-2-hydroxyacetohydrazide. To a stirring solution of ethyl 2-[3-(3-bromophenyl)oxetan-3-yl]-2-hydroxyacetate (81 g, 257 mmol) in ethanol (810 mL) was added hydrazine hydrate (130 mL, 2.57 mol, 85%) dropwise at rt under nitrogen atmosphere. The mixture was stirred for 12 h. The mixture was concentrated under reduced pressure until most of the ethanol was removed. The precipitated solids were collected by filtration and washed with diethyl ether (2×) to afford the title compound (63 g, 81%) as a white solid.

Step 4: Synthesis 2-[3-(3-bromophenyl)oxetan-3-yl]-2-hydroxy-N-[(methylcarbamothioyl)amino]acetamide. To a stirred solution of 2-[3-(3-bromophenyl)oxetan-3-yl]-2-hydroxyacetohydrazide (63 g, 209 mmol) in THF (441 mL) was added isothiocyanatomethane (18 g, 251 mmol) at rt under nitrogen atmosphere. The mixture was stirred for 12 h at 50° C. under nitrogen atmosphere. The mixture was concentrated under reduced pressure. The solids ware washed with diethyl ether to afford the title compound (75 g, 96%) as a white solid.

Step 5: Synthesis [3-(3-bromophenyl)oxetan-3-yl](4-methyl-5-sulfanyl-4H-1,2,4-triazol-3-yl)methanol. To a stirring solution of 2-[3-(3-bromophenyl)oxetan-3-yl]-2-hydroxy-N-[(methylcarbamothioyl)amino]acetamide (75 g, 200. mmol) was added sodium hydroxide (451 mL, 451 mmol) at rt. The mixture was stirred for 12 h at rt. The mixture was acidified to pH ˜5 with 4N aqueous hydrochloric acid. The aqueous layer was extracted with CH₂Cl₂ (3×) followed by General Work-up Procedure 1 to afford the title compound (63 g, 88%) as a white solid.

Step 6: Synthesis of [3-(3-bromophenyl)oxetan-3-yl](4-methyl-4H-1,2,4-triazol-3-yl)methanol. To a stirring solution of [3-(3-bromophenyl)oxetan-3-yl](4-methyl-5-sulfanyl-4H-1,2,4-triazol-3-yl)methanol (63 g, 176 mmol) in DCM (504 mL) were added acetic acid (252 mL) and hydrogen peroxide (189 mL, 30% aq.) dropwise at 0° C. The mixture was stirred for 2 h at rt. The mixture was basified to pH=8 with saturated aqueous sodium bicarbonate. The mixture was concentrated under reduced pressure. The solid was collected by filtration and washed with water to afford the title compound (50 g, 87%) as a white solid.

Step 7: Synthesis of 3-[[3-(3-bromophenyl)oxetan-3-yl](fluoro)methyl]-4-methyl-4H-1,2,4-triazole. To a stirring mixture of [3-(3-bromophenyl)oxetan-3-yl](4-methyl-4H-1,2,4-triazol-3-yl)methanol (40 g, 123 mmol) in DCM (400 mL) was added DAST (119 g, 740 mmol) dropwise at 0° C. under nitrogen atmosphere. The mixture was stirred for 8 h at 0° C. The mixture was basified to pH8 with saturated aqueous sodium bicarbonate. The mixture was filtered and the filtrate was extracted with CH₂Cl₂ (3×). The combined organic layers were dried and concentrated under reduced pressure to afford the title compound (25.1 g, 62.37%) as a yellow solid. ¹H NMR (300 MHz, DMSO-d₆) δ 8.41 (s, 2H), 7.50-7.40 (m, 4H), 7.32-7.17 (m, 4H), 6.35 (s, 1H), 6.19 (s, 1H), 5.27 (d, J=6.9 Hz, 2H), 5.12 (ddd, J=17.7, 6.7, 1.8 Hz, 4H), 4.76 (dd, J=6.3, 4.0 Hz, 2H), 3.32 (s, 7H); LCMS: C₁₃H₁₃BrFN₃O₂ requires: 325, found: m/z=326 [M+H]⁺.

### Example AG and Example AH

### (S)-3-(3-(fluoro(4-methyl-4H-1,2,4-triazol-3-yl)methyl)oxetan-3-yl)aniline (AG) and (R)-3-(3-(fluoro(4-methy-4H-1,2,4-triazol-3-yl)methyl)oxetan-3-yl)aniline (AH)

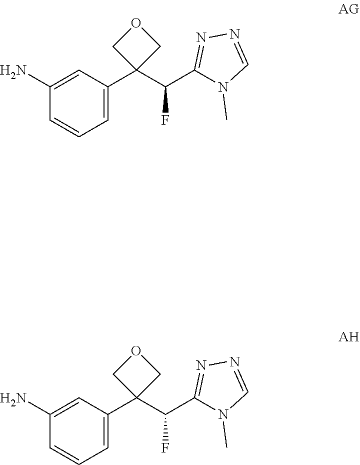 [SMILES: Cn1cnnc1[C@@H](F)C1(c2cccc(N)c2)COC1 ; Cn1cnnc1[C@H](F)C1(c2cccc(N)c2)COC1]

Step 1: Synthesis of ethyl 2-[3-(3-bromophenyl)oxetan-3-yl]-2-hydroxyacetate. To a solution of ethyl 2-[3-(3-bromophenyl)oxetan-3-yl]acetate (13.0 g, 43.6 mmol) in dry THF (150 mL) was KHMDS (65.4 mL, 65.4 mmol, 1M in THF) dropwise at −78° C. and stirred for 40 min. Then to the above mixture was added a solution of 3-phenyl-2-(phenylsulfonyl)-1,2-oxaziridine (14.8 g, 56.7 mmol) in THF (50 mL) dropwise below −65° C. The solution was stirred for 3 h below −60° C. The mixture was then warmed to rt and quenched by the addition of saturated NH₄Cl aqueous solution followed by General Work-up Procedure 1. The residue was purified by chromatography A to afford the title compound (10.3 g, 75%) as a white solid.

Step 2: Synthesis of 2-[3-(3-bromophenyl)oxetan-3-yl]-2-hydroxyacetohydrazide. To a solution of ethyl 2-[3-(3-bromophenyl)oxetan-3-yl]-2-hydroxyacetate (10.3 g, 32.8 mmol) in ethanol (100 mL) was added hydrazine hydrate (20.5 g, 328.0 mmol, 80% wt). The solution was stirred at 50° C. for 16 h. The solids were collected by filtration and triturated with EtOAc/petroleum ether to afford the title compound, which was used in the next step without purification.

Step 3: Synthesis of 2-[3-(3-bromophenyl)oxetan-3-yl]-2-hydroxy-N-[(methylcarbamothioyl)amino]acetamide. To a solution of 2-[3-(3-bromophenyl)oxetan-3-yl]-2-hydroxyacetohydrazide (8.91 g, 29.7 mmol) in THF (60 mL) was added isothiocyanatomethane (2.6 g, 35.6 mmol). The solution was stirred at 50° C. for 16 h. The solvent was removed under vacuum. The residue was triturated with DCM/methanol (1/10) to afford the title compound as a white solid, which was used in the next step without purification.

Step 4: Synthesis of [3-(3-bromophenyl)oxetan-3-yl](4-methyl-5-sulfanyl-4H-1,2,4-triazol-3-yl)methanol. A solution of 2-[3-(3-bromophenyl)oxetan-3-yl]-2-hydroxy-N-[(methylcarbamothioyl)amino]acetamide in sodium hydroxide (80 mL, 1 M) was stirred at rt for 16 h. After the reaction was completed, the pH value of the mixture was adjusted to 5 with hydrochloric acid (4 N) followed by General Work-up Procedure 1 to afford the title compound as a white solid, which was used in the next step without purification.

Step 5: Synthesis of [3-(3-bromophenyl)oxetan-3-yl](4-methyl-4H-1,2,4-triazol-3-yl)methanol. To a solution of [3-(3-bromophenyl)oxetan-3-yl](4-methyl-5-sulfanyl-4H-1,2,4-triazol-3-yl)methanol in DCM (80 mL) and acetic acid (40 mL) was added hydrogen peroxide (10 mL, 30%). The solution was stirred at rt for 2 h. The reaction was then quenched by the addition of saturated sodium bicarbonate aqueous solution and extracted with DCM (3×) followed by General Work-up Procedure 1. The residue was purified by chromatography B to afford the title compound (5.8 g, 56% over 4 steps) as a white solid

Step 6: Synthesis of 3-([3-[(3E)-4-bromopenta-1,3-dien-2-yl]oxetan-3-yl](fluoro)methyl)-4-methyl-4H-1,2,4-triazole. To a solution of [3-(3-bromophenyl)oxetan-3-yl](4-methyl-4H-1,2,4-triazol-3-yl)methanol (5.8 g, 17.9 mmol) in DCM (60 mL) was added DAST (17.6 g, 107 mmol) dropwise at 0° C. The mixture was stirred at 0° C. for 4 h. The reaction was then quenched by the addition of saturated sodium bicarbonate aqueous solution and the aqueous phase was extracted with DCM (3×) followed by General Work-up Procedure 1. The residue was purified by chromatography B to afford the title compound (4.3 g, 74%) as a white solid.

Step 7: Synthesis of (S)-3-(3-(fluoro(4-methyl-4H-1,2,4-triazol-3-yl)methyl)oxetan-3-yl)aniline and (R)-3-(3-(fluoro(4-methyl-4H-1,2,4-triazol-3-yl)methyl)oxetan-3-yl)aniline. To a degassed solution of 3-[[3-(3-bromophenyl)oxetan-3-yl](fluoro)methyl]-4-methyl-4H-1,2,4-triazole (280 mg, 0.86 mmol) in acetonitrile (10 mL) and ammonia (10 mL) was added Cu₂O (24 mg, 0.17 mmol). The solution was stirred at 100° C. for 12 h in a sealed tube. The solids were filtered out and the filtrate was concentrated under vacuum. The residue was purified by reverse phase Chromatography C to afford racemic 3-(3-(fluoro(4-methyl-4H-1,2,4-triazol-3-yl)methyl)oxetan-3-yl)aniline (290 mg) as a white solid, which was further separated by prep-chiral HPLC with the following conditions: [Column, CHIRALPAK IG, 20*250 mm, 5 um; mobile phase, Hex (8 mmol/L NH₃.MeOH) and EtOH (hold 50% EtOH in 14 min); Detector, UV 220 nm] to afford (S)-3-(3-(fluoro(4-methyl-4H-1,2,4-triazol-3-yl)methyl)oxetan-3-yl)aniline (AG, 120.5 mg) with shorter retention time on chiral HPLC as an off-white solid and (R)-3-(3-(fluoro(4-methyl-4H-1,2,4-triazol-3-yl)methyl)oxetan-3-yl)aniline (AH, 107.1 mg) with longer retention time on chiral HPLC as an off-white solid.

(S)-3-(3-(fluoro(4-methyl-4H-1,2,4-triazol-3-yl)methyl)oxetan-3-yl)aniline: ¹H NMR (400 MHz, DMSO-d₆) δ 8.36 (s, 1H), 6.92 (m, 1H), 6.43 (m, 1H), 6.26-6.04 (m, 3H), 5.28 (m, 1H), 5.15-5.01 (m, 3H), 4.93 (m, 1H), 4.72 (m, 1H), 3.07 (s, 3H); LCMS: C₁₃H₁₅FN₄O requires: 262, found: m/z=263 [M+H]⁺.

(R)-3-(3-(fluoro(4-methyl-4H-1,2,4-triazol-3-yl)methyl)oxetan-3-yl)aniline: ¹H NMR (400 MHz, DMSO-d₆) δ 8.36 (s, 1H), 6.92 (m, 1H), 6.43 (m, 1H), 6.27-6.06 (m, 3H), 5.28 (m, 1H), 5.12-5.02 (m, 3H), 4.93 (m, 1H), 4.72 (m, 1H), 3.07 (s, 3H). LCMS: C₁₃H₁₅FN₄O requires: 262, found: m/z=263 [M+H]⁺.

### General Procedure 1

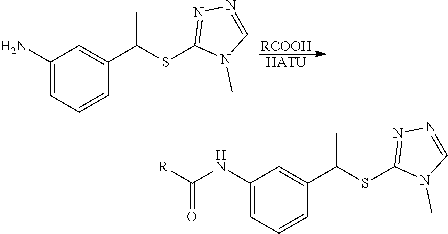

A mixture of 3-(1-(4-methyl-4H-1,2,4-triazol-3-ylthio)ethyl)benzenamine (A, see Example A) (100 mg, 0.43 mmol), precursor carboxylic acid (0.51 mmol), HATU (241 mg, 0.64 mmol) and DIPEA (109 mg, 0.85 mmol) in DMF (3 mL) was stirred at 25° C. for 1-16 h. The mixture was diluted by the addition of water (20 mL) and extracted with EtOAc (20 mL×3). All the organic layers were combined, dried, and concentrated. The residue was purified by prep-HPLC or flash column chromatography to afford the desired product.

### General Procedure 2

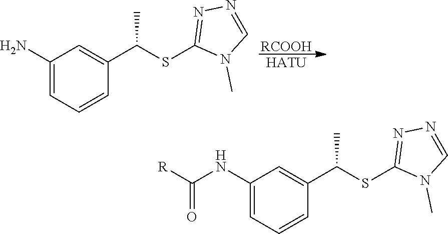

To a mixture of (S)-3-(1-(4-methyl-4H-1,2,4-triazol-3-ylthio)ethyl)aniline (A-a, see Example A) (100 mg, 0.43 mmol), precursor carboxylic acid (0.51 mmol), HATU (241 mg, 0.64 mmol) and DIPEA (109 mg, 0.85 mmol) in DMF (3 mL) was stirred at 25° C. for 1-24 h. The mixture was diluted by the addition of water and extracted with EtOAc. All the organic layers were combined, optionally washed with water or brine, dried, filtered and concentrated. The residue was purified by prep-HPLC or flash column chromatography to afford the desired product.

### General Procedure 3

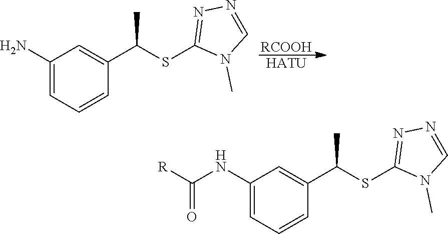

To a mixture of (R)-3-(1-(4-methyl-4H-1,2,4-triazol-3-ylthio)ethyl)aniline (A-b, see Example A) (100 mg, 0.43 mmol), precursor carboxylic acid (0.51 mmol), HATU (241 mg, 0.64 mmol) and DIPEA (109 mg, 0.85 mmol) in DMF (3 mL) was stirred at 25° C. for 16 h. The mixture was diluted by the addition of water (20 mL) and extracted with EtOAc (20 mL×3). All the organic layers were combined, dried, and concentrated. The residue was purified by prep-HPLC or flash column chromatography to afford the product.

### General Procedure 4: (See Example 98, Step 3)

### General Procedure 5: (see Example 138)

### General Procedure 6

To a mixture of (R)-3-(1-(4-methyl-4H-1,2,4-triazol-3-yl)propan-2-yl)aniline (100 mg, 0.46 mmol), carboxylic acid (0.56 mmol), HATU (262 mg, 0.69 mmol) and DIPEA (118 mg, 0.92 mmol) in DMF (3 mL) was stirred at 25° C. for 2-24 h. The mixture was diluted by the addition of water and extracted with EtOAc. The organic layers were combined, optionally washed with water and/or brine, dried, filtered and concentrated. The residue was purified by either prep-HPLC or flash column chromatography to afford the product.

### General Procedure 7

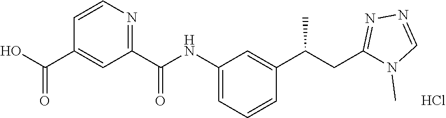 [SMILES: C[C@H](Cc1nncn1C)c1cccc(NC(=O)c2cc(C(=O)O)ccn2)c1 ; Cl]

To a mixture of 2-([3-[(2R)-1-(4-methyl-4H-1,2,4-triazol-3-yl)propan-2-yl]phenyl]carbamoyl)pyridine-4-carboxylic acid OR (R)-2-(3-(1-(4-methyl-4H-1,2,4-triazol-3-yl)propan-2-yl)phenylcarbamoyl)isonicotinic acid OR J-a hydrogen chloride (250 mg, 0.68 mmol), Amine (0.82 mmol), HATU (390 mg, 1.03 mmol) and DIPEA (265 mg, 2.05 mmol) in DMF (5 mL) was stirred at rt for 2-18 h and monitored by LCMS. The mixture was diluted by the addition of water and extracted with EtOAc. The organic layers were combined, optionally washed with brine, dried, and concentrated. The residue was purified by Prep-HPLC or flash column chromatography, optionally preceded by an initial purification via flash column chromatography.

### General Procedure 1-G

A mixture of amine, acid, HATU, and DIPEA in DMF was stirred at 0-RT ° C. for 1-24 h. The mixture was purified by prep-HPLC purification methods or flash column chromatography, typically preceded by one or more of the following workup procedures: quenching with water, solvent extraction, wash with water or brine, drying over sodium sulfate, filtration, and concentration under reduced pressure.

### General Work-Up Procedure 1

Aqueous solutions were extracted with EtOAc 2-3 times. The combined organic extract was dried, over anhydrous magnesium or sodium sulfate, or was washed with brine or saturated ammonium chloride aqueous solution before drying, filtration, and concentration under vacuum.

### Purification Procedures

Preparative-scale chiral supercritical fluid chromatography was performed using various CHIRALPAK columns, such as CHIRALPAK AS-H, CHIRALPAK AD-H, or CHIRALPAK IG, using solvent systems such as CO₂/MeOH, CO₂/EtOH, or CO₂/(MeOH+ acetonitrile).

Preparative-scale chiral HPLC was performed using various CHIRALPAK columns, such as CHIRALPAK IA, CHIRAL ART Cellulose-SB, CHIRALPAK IF, CHIRALPAK IG, using solvent systems such as hexane/methanol, hexane/ethanol, (hexane+dichloromethane)/ethanol, MTBE/methanol, MTBE/ethanol, (hexane-8 mmol/L NH₃)/methanol, Mobile Phase B: ethanol and hexane/IPA.

Preparative-scale HPLC was performed using columns such as SunFire Prep C18 OBD, XBridge Prep OBD C18 and XBridge Shield RP18 OBD, using solvent systems such as (water-0.1% formic acid)/acetonitrile, (water-10 mmol/L NH₄HCO₃)/acetonitrile, and (water-10 mmol/L NH₄HCO₃)/acetonitrile.

### Example 1

### General Procedure 1

To a mixture of A (see Example A) (100 mg, 0.43 mmol), acid (0.51 mmol), HATU (241 mg, 0.64 mmol) and DIPEA (109 mg, 0.85 mmol) in DMF (3 mL) was stirred at 25° C. for 16 h. The mixture was diluted by the addition of water (20 mL). General Work-up Procedure 1 was followed. The residue was purified by Chromatography C to afford the desired product.

### 3-Chloro-N-(3-(1-((4-methyl-4H-1,2,4-triazol-3-yl)thio)ethyl)phenyl)benzamide (1)

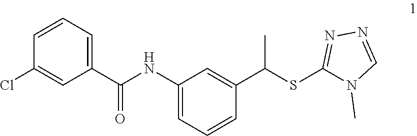 [SMILES: CC(Sc1nncn1C)c1cccc(NC(=O)c2cccc(Cl)c2)c1]

Followed General procedure 1 to afford 1 (62.9 mg, 40%) as a colorless solid. 1H NMR (400 MHz, DMSO-d₆) δ 10.37 (s, 1H), 8.56 (s, 1H), 8.02 (t, J=1.9 Hz, 1H), 7.93 (d, J=8.0 Hz, 1H), 7.77 (t, J=1.9 Hz, 1H), 7.71-7.66 (m, 2H), 7.58 (t, J=8.0 Hz, 1H), 7.30 (t, J=8.0 Hz, 1H), 7.04 (d, J=8.0 Hz, 1H), 4.68 (q, J=6.8 Hz, 1H), 3.40 (s, 3H), 1.66 (d, J=6.8 Hz, 3H). MS (ESI) calc'd for (C₁₈H₁₇ClN₄OS) [M+H]⁺, 373.1; found, 373.1.

### Example 2: 4-Chloro-N-(3-(1-(4-methyl-4H-1,2,4-triazol-3-ylthio)ethyl)phenyl)benzamide (2)

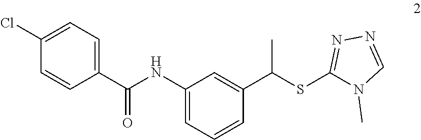 [SMILES: CC(Sc1nncn1C)c1cccc(NC(=O)c2ccc(Cl)cc2)c1]

Followed General procedure 1 to afford 2 (90.4 mg, 57%). ¹H NMR (400 MHz, DMSO-d₆) δ 10.33 (s, 1H), 8.55 (s, 1H), 8.04-7.93 (m, 2H), 7.78 (t, J=1.9 Hz, 1H), 7.72-7.70 (m, 1H), 7.67-7.54 (m, 2H), 7.29 (t, J=8.0 Hz, 1H), 7.03 (d, J=8.0 Hz, 1H), 4.67 (q, J=6.8 Hz, 1H), 3.40 (s, 3H), 1.66 (d, J=12 Hz, 3H). MS (ESI) calc'd for (C₁₈H₁₇ClN₄OS) [M+H]⁺, 373.1; found, 373.1.

### Example 3: 2-Methoxy-N-(3-[1-[(4-methyl-4H-1,2,4-triazol-3-yl)sulfanyl]ethyl]phenyl)benzamide (3)

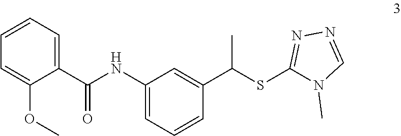 [SMILES: COc1ccccc1C(=O)Nc1cccc(C(C)Sc2nncn2C)c1]

Followed General procedure 1 to afford 3 (50.1 mg, 32%). ¹H NMR (400 MHz, DMSO-d₆) δ 10.13 (s, 1H), 8.57 (s, 1H), 7.76 (t, J=2.0 Hz, 1H), 7.67-7.64 (m, 2H), 7.56-7.51 (m, 1H), 7.27 (t, J=8.0 Hz, 1H), 7.19 (d, J=8.4 Hz, 1H), 7.13-6.98 (m, 2H), 4.67 (q, J=6.8 Hz, 1H), 3.91 (s, 3H), 3.41 (s, 3H), 1.65 (d, J=6.8 Hz, 3H). MS (ESI) calc'd for (C₁₉H₂₀N₄O₂S) [M+H]⁺, 369.1; found, 369.1.

### Example 4: 3-Methoxy-N-(3-[1-[(4-methyl-4H-1,2,4-triazol-3-yl)sulfanyl]ethyl]phenyl)benzamide (4)

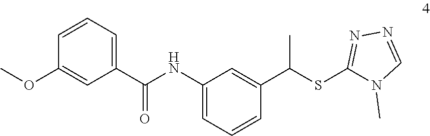 [SMILES: COc1cccc(C(=O)Nc2cccc(C(C)Sc3nncn3C)c2)c1]

Followed General procedure 1 to afford 4 (87.3 mg, 37%). ¹H NMR (400 MHz, DMSO-d₆) δ 10.24 (s, 1H), 8.56 (s, 1H), 7.79 (t, J=2.0 Hz, 1H), 7.75-7.72 (m, 1H), 7.57-7.55 (m, 1H), 7.52-7.41 (m, 2H), 7.29 (t, J=8.0 Hz, 1H), 7.21-7.17 (m, 1H), 7.06-7.03 (m, 1H), 4.67 (q, J=6.8 Hz, 1H), 3.85 (s, 3H), 3.40 (s, 3H), 1.66 (d, J=6.8 Hz, 3H). MS (ESI) calc'd for (C₁₉H₂₀N₄O₂S) [M+H]⁺, 369.1; found, 369.1.

### Example 5: 4-methoxy-N-(3-[1-[(4-methyl-4H-1,2,4-triazol-3-yl)sulfanyl]ethyl]phenyl)benzamide (5)

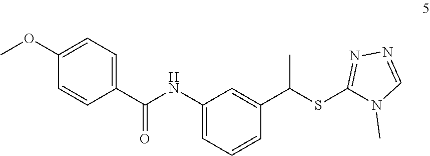 [SMILES: COc1ccc(C(=O)Nc2cccc(C(C)Sc3nncn3C)c2)cc1]

Followed General procedure 1 to afford 5 (122.9 mg, 52%). ¹H NMR (400 MHz, DMSO-d₆) δ 10.11 (s, 1H), 8.57 (s, 1H), 8.01-7.94 (m, 2H), 7.79 (t, J=2.0 Hz, 1H), 7.76-7.69 (m, 1H), 7.27 (t, J=8.0 Hz, 1H), 7.12-7.04 (m, 2H), 7.10-7.00 (m, 1H), 4.67 (q, J=6.8 Hz, 1H), 3.85 (s, 3H), 3.40 (s, 3H), 1.66 (d, J=6.8 Hz, 3H). MS (ESI) calc'd for (C₁₉H₂₀N₄O₂S) [M+H]⁺, 369.1; found, 369.1.

### Example 6: N-(3-(1-((4-Methyl-4H-1,2,4-triazol-3-yl)thio)ethyl)phenyl)picolinamide (6)

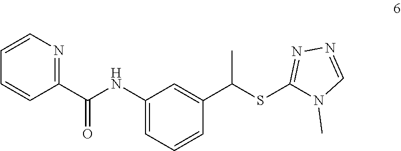 [SMILES: CC(Sc1nncn1C)c1cccc(NC(=O)c2ccccn2)c1]

Followed General procedure 1 to afford 6 (74.4 mg, 51%). ¹H NMR (400 MHz, DMSO-d₆) δ 10.64 (s, 1H), 8.75 (d, J=4.8 Hz, 1H), 8.56 (s, 1H), 8.17 (d, J=8.0 Hz, 1H), 8.09 (t, J=8.0 Hz, 1H), 7.97 (t, J=1.9 Hz, 1H), 7.84-7.82 (m, 1H), 7.69-7.67 (m, 1H), 7.30 (t, J=8.0 Hz, 1H), 7.04 (d, J=8.0 Hz, 1H), 4.67 (q, J=6.8 Hz, 1H), 3.40 (s, 3H), 1.67 (d, J=7.2 Hz, 3H). MS (ESI) calc'd for (C₁₇H₁₇N₅OS) [M+H]⁺, 340.1; found, 340.1.

### Example 7: N-(3-(1-((4-Methyl-4H-1,2,4-triazol-3-yl)thio)ethyl)phenyl)pyrimidine-4-carboxamide (7)

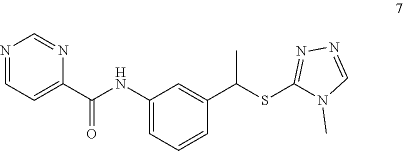 [SMILES: CC(Sc1nncn1C)c1cccc(NC(=O)c2ccncn2)c1]

Followed General procedure 1 to afford 7 (94.5 mg, 65%). ¹H NMR (300 MHz, DMSO-d₆) δ 10.85 (s, 1H), 9.44 (d, J=1.5 Hz, 1H), 9.15 (d, J=5.1 Hz, 1H), 8.55 (s, 1H), 8.16-8.13 (m, 1H), 7.97 (m=1.8 Hz, 1H), 7.83 (d, J=7.8 Hz, 1H), 7.34-7.30 (m, 1H), 7.07 (d, J=7.8 Hz, 1H), 4.71-4.64 (m, 1H), 3.39 (s, 3H), 1.67 (d, J=6.9 Hz, 3H). MS (ESI) calculated for (C₁₆H₁₆N₆OS) [M+H]⁺, 341.1; found, 341.1.

### Example 8: N-(3-(1-((4-Methyl-4H-1,2,4-triazol-3-yl)thio)ethyl)phenyl)isoquinoline-3-carboxamide (8)

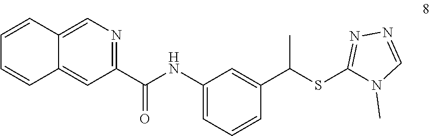 [SMILES: CC(Sc1nncn1C)c1cccc(NC(=O)c2cc3ccccc3cn2)c1]

Followed General procedure 1 to afford 8 (92.3 mg, 54%). ¹H NMR (400 MHz, DMSO-d₆) δ 10.77 (s, 1H), 9.48 (s, 1H), 8.71 (s, 1H), 8.56 (s, 1H), 8.32 (d, J=8.0 Hz, 1H), 8.26 (d, J=8.4 Hz, 1H), 8.00 (s, 1H), 7.95-7.84 (m, 3H), 7.32 (t, J=8.0 Hz, 1H), 7.05 (d, J=8.0 Hz, 1H), 4.69 (q, J=6.8 Hz, 1H), 3.41 (s, 3H), 1.69 (d, J=6.8 Hz, 3H). MS (ESI) calc'd for (C₂₁H₁₉N₅OS) [M+H]⁺, 390.2; found, 390.2.

### General Procedure 2:

To a mixture of A-a (100 mg, 0.43 mmol), acid (0.51 mmol), HATU (241 mg, 0.64 mmol) and DIPEA (109 mg, 0.85 mmol) in DMF (3 mL) was stirred at 25° C. for 16 h. The mixture was diluted by the addition of water (20 mL). General Work-up Procedure 1 was followed. The residue was purified by prep-HPLC or flash column chromatography to afford the desired product.

### Example 8a: (S)-N-(3-(1-((4-Methyl-4H-1,2,4-triazol-3-yl)thio)ethyl)phenyl)isoquinoline-3-carboxamide (8a)

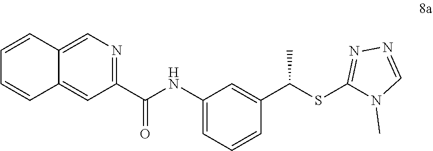 [SMILES: C[C@H](Sc1nncn1C)c1cccc(NC(=O)c2cc3ccccc3cn2)c1]

Followed General procedure 2 to afford 8a (110.3 mg, 66%). ¹H NMR (300 MHz, DMSO-d₆) δ 10.78 (s, 1H), 9.49 (s, 1H), 8.71 (s, 1H), 8.58 (s, 1H), 8.31-8.25 (m, 2H), 8.00 (d, J=2.1 Hz, 1H), 7.98-7.81 (m, 3H), 7.32 (t, J=7.8 Hz, 1H), 7.05 (d, J=7.8 Hz, 1H), 4.69 (q, J=6.9 Hz, 1H), 3.41 (s, 3H), 1.69 (d, J=6.9 Hz, 3H). MS (ESI) calculated for (C₂₁H₁₉N₅OS) [M+H]⁺, 390.1; found, 390.2.

### General Procedure 3:

To a mixture of A-b (100 mg, 0.43 mmol), acid (0.51 mmol), HATU (241 mg, 0.64 mmol) and DIPEA (109 mg, 0.85 mmol) in DMF (3 mL) was stirred at 25° C. for 16 h. The mixture was diluted by the addition of water (20 mL). General Work-up Procedure 1 was followed. The residue was purified by prep-HPLC or Chromatography to afford the title compound.

### Example 8b: (R)-N-(3-(1-((4-methyl-4H-1,2,4-triazol-3-yl)thio)ethyl)phenyl)isoquinoline-3-carboxamide (8b)

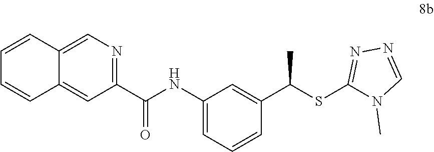 [SMILES: C[C@@H](Sc1nncn1C)c1cccc(NC(=O)c2cc3ccccc3cn2)c1]

Followed General procedure 3 to afford 8b (126 mg, 20%). MS (ESI) calc'd for (C₂₁H₁₉N₅OS) [M+H]⁺, 390.1; found, 390.0. ¹H NMR (300 MHz, DMSO-d₆) δ 10.77 (s, 1H), 9.48 (s, 1H), 8.72 (s, 1H), 8.55 (s, 1H), 8.33-8.24 (m, 2H), 8.00-7.83 (m, 4H), 7.34 (t, J=7.8 Hz, 1H), 7.04 (d, J=7.8 Hz, 1H), 4.70 (q, J=6.9 Hz, 1H), 3.42 (s, 3H), 1.69 (d, J=6.9 Hz, 3H).

### Example 9: N-(3-(1-((4-Methyl-4H-1,2,4-triazol-3-yl)thio)ethyl)phenyl)quinoline-2-carboxamide (9)

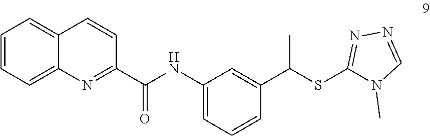 [SMILES: CC(Sc1nncn1C)c1cccc(NC(=O)c2ccc3ccccc3n2)c1]

Followed General procedure 1 to afford 9 (75.1 mg, 45%). ¹H NMR (400 MHz, DMSO-d₆) δ 10.77 (s, 1H), 8.65 (d, J=8.5 Hz, 1H), 8.57 (s, 1H), 8.37-8.22 (m, 2H), 8.14 (d, J=8.4 Hz, 1H), 8.02-7.86 (m, 3H), 7.79-7.75 (m, 1H), 7.34 (t, J=8.0 Hz, 1H), 7.07 (d, J=8.0 Hz, 1H), 4.71 (q, J=6.8 Hz, 1H), 3.41 (s, 3H), 1.70 (d, J=6.8 Hz, 3H). MS (ESI) calc'd for (C₂₁H₁₉N₅OS) [M+H]⁺, 390.2; found, 390.2.

### (S)-N-(3-(1-((4-Methyl-4H-1,2,4-triazol-3-yl)thio)ethyl)phenyl)quinoline-3-carboxamide (9a)

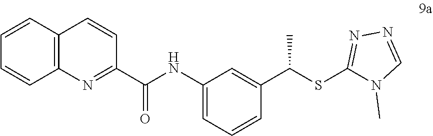 [SMILES: C[C@H](Sc1nncn1C)c1cccc(NC(=O)c2ccc3ccccc3n2)c1]

Followed General procedure 2 (see Example 8) to afford 9a (100.1 mg, 60%). ¹H NMR (400 MHz, DMSO-d₆) δ 10.77 (s, 1H), 8.65 (d, J=8.4 Hz, 1H), 8.56 (s, 1H), 8.27 (t, J=8.4 Hz, 2H), 8.14 (d, J=8.0 Hz, 1H), 8.03-7.98 (m, 1H), 7.97-7.86 (m, 2H), 7.78 (t, J=7.6 Hz, 1H), 7.34 (t, J=8.0 Hz, 1H), 7.06 (d, J=7.6 Hz, 1H), 4.71 (q, J=6.8 Hz, 1H), 3.41 (s, 3H), 1.70 (d, J=6.8 Hz, 3H). MS (ESI) calculated for (C₂₁H₁₉N₅OS) [M+H]⁺, 390.1; found, 390.1.

### (R)-N-(3-(1-((4-methyl-4H-1,2,4-triazol-3-yl)thio)ethyl)phenyl)quinoline-2-carboxamide (9b)

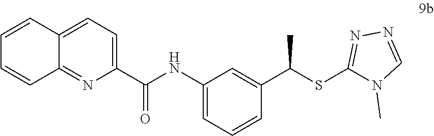 [SMILES: C[C@@H](Sc1nncn1C)c1cccc(NC(=O)c2ccc3ccccc3n2)c1]

Followed General procedure 3 (see Example 8) to afford 9b (264 mg, 57%). MS (ESI) calculated for (C₂₁H₁₉N₅OS) [M+H]⁺, 390.1; found, 390.1. ¹H NMR (300 MHz, DMSO-d₆) δ 10.78 (s, 1H), 8.70-8.65 (m, 1H), 8.57 (s, 1H), 8.33-8.21 (m, 2H), 8.16-8.14 (m, 1H), 8.00 (t, J=1.8 Hz, 1H), 7.98-7.86 (m, 2H), 7.81-7.78 (m, 1H), 7.34 (t, J=7.8 Hz, 1H), 7.10-7.07 (m, 1H), 4.71 (q, J=6.9 Hz, 1H), 3.41 (s, 3H), 1.70 (d, J=6.9 Hz, 3H).

### Example 10: N-(3-(1-((4-Methyl-4H-1,2,4-triazol-3-yl)thio)ethyl)phenyl)quinoline-3-carboxamide (10)

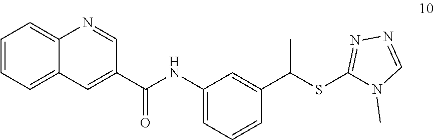 [SMILES: CC(Sc1nncn1C)c1cccc(NC(=O)c2cnc3ccccc3c2)c1]

Followed General procedure 1 to afford 10 (106.1 mg, 64%). ¹H NMR (400 MHz, DMSO-d₆) δ 10.64 (s, 1H), 9.37 (s, 1H), 8.98 (s, 1H), 8.58 (s, 1H), 8.18-8.15 (m, 2H), 7.94-7.90 (m, 1H), 7.83 (s, 1H), 7.80-7.71 (m, 2H), 7.34 (t, J=8.0 Hz, 1H), 7.07 (d, J=8.0 Hz, 1H), 4.71 (q, J=6.8 Hz, 1H), 3.42 (s, 3H), 1.68 (d, J=6.8 Hz, 3H). MS (ESI) calc'd for (C₂₁H₁₉N₅OS) [M+H]⁺, 390.1; found, 390.2.

### Example 11: N-(3-[1-[(4-Methyl-4H-1,2,4-triazol-3-yl)sulfanyl]ethyl]phenyl)quinoline-6-carboxamide (11)

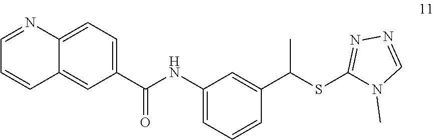 [SMILES: CC(Sc1nncn1C)c1cccc(NC(=O)c2ccc3ncccc3c2)c1]

Followed General procedure 1 to afford 11 (103.0 mg, 61%). ¹H NMR (400 MHz, DMSO-d₆) δ 10.55 (s, 1H), 9.08-9.05 (m, 1H), 8.67 (d, J=2.0 Hz, 1H), 8.59 (d, J=6.8 Hz, 2H), 8.35-8.29 (m, 1H), 8.16 (d, J=8.8 Hz, 1H), 7.87-7.74 (m, 2H), 7.82-7.69 (m, 1H), 7.33 (t, J=8.0 Hz, 1H), 7.15-7.06 (m, 1H), 4.70 (q, J=6.8 Hz, 1H), 3.42 (s, 3H), 1.68 (d, J=6.8 Hz, 3H). MS (ESI) calc'd for (C₂₁H₁₉N₅OS) [M+H]⁺, 390.1; found, 390.1.

### Example 12: N-(3-[1-[(4-Methyl-4H-1,2,4-triazol-3-yl)sulfanyl]ethyl]phenyl)isoquinoline-7-carboxamide (12)

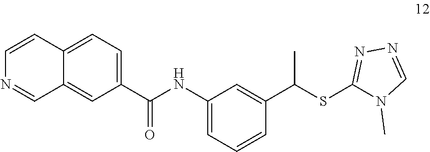 [SMILES: CC(Sc1nncn1C)c1cccc(NC(=O)c2ccc3ccncc3c2)c1]

Followed General procedure 1 to afford 12 (73.8 mg, 44%). ¹H NMR (400 MHz, DMSO-d₆) δ 10.58 (s, 1H), 9.52 (s, 1H), 8.86-8.78 (m, 1H), 8.64 (d, J=5.6 Hz, 1H), 8.57 (s, 1H), 8.45-8.30 (m, 1H), 8.20-8.09 (m, 1H), 7.98 (d, J=5.6 Hz, 1H), 7.86-7.71 (m, 2H), 7.33 (t, J=8.0 Hz, 1H), 7.10-7.06 (m, 1H), 4.70 (q, J=6.8 Hz, 1H), 3.42 (s, 3H), 1.68 (d, J=6.8 Hz, 3H). MS (ESI) calc'd for (C₂₁H₁₉N₅OS) [M+H]⁺, 390.1; found, 390.1.

### Example 13: N-(3-[1-[(4-Methyl-4H-1,2,4-triazol-3-yl)sulfanyl]ethyl]phenyl)quinoline-7-carboxamide (13)

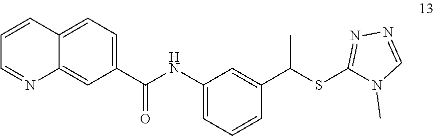 [SMILES: CC(Sc1nncn1C)c1cccc(NC(=O)c2ccc3cccnc3c2)c1]

Followed General procedure 1 to afford 13 (101.8 mg, 61%). ¹H NMR (400 MHz, DMSO-d₆) δ 10.60 (s, 1H), 9.16-9.05 (m, 1H), 8.71 (d, J=1.6 Hz, 1H), 8.59 (d, J=1.2 Hz, 1H), 8.54-8.43 (m, 1H), 8.20-8.08 (m, 2H), 7.90-7.74 (m, 2H), 7.75-7.68 (m, 1H), 7.32 (t, J=8.0 Hz, 1H), 7.15-7.06 (m, 1H), 4.70 (q, J=6.8 Hz, 1H), 3.42 (s, 3H), 1.68 (d, J=6.8 Hz, 3H). MS (ESI) calc'd for (C₂₁H₁₉N₅OS) [M+H]⁺, 390.1; found, 390.1.

### Example 14: N-(3-[1-[(4-Methyl-4H-1,2,4-triazol-3-yl)sulfanyl]ethyl]phenyl)isoquinoline-6-carboxamide (14)

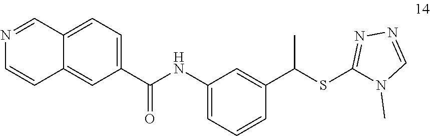 [SMILES: CC(Sc1nncn1C)c1cccc(NC(=O)c2ccc3cnccc3c2)c1]

Followed General procedure 1 to afford 14 (80.7 mg, 49%). ¹H NMR (400 MHz, DMSO-d₆) δ 10.60 (s, 1H), 9.46 (s, 1H), 8.67-8.50 (m, 3H), 8.30 (d, J=8.4 Hz, 1H), 8.21-8.10 (m, 1H), 8.02 (d, J=5.6 Hz, 1H), 7.86-7.73 (m, 2H), 7.33 (t, J=8.0 Hz, 1H), 7.15-7.06 (m, 1H), 4.70 (q, J=6.8 Hz, 1H), 3.41 (s, 3H), 1.68 (d, J=6.8 Hz, 3H). MS (ESI) calc'd for (C₂₁H₁₉N₅OS) [M+H]⁺, 390.1; found, 390.1.

### Example 15: 7-Methyl-N-(3-(1-(4-methyl-4H-1,2,4-triazol-3-ylthio)ethyl)phenyl)quinoline-3-carboxamide (15)

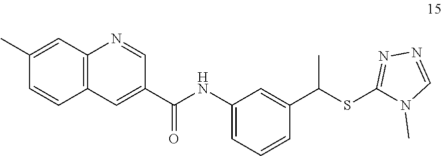 [SMILES: Cc1ccc2cc(C(=O)Nc3cccc(C(C)Sc4nncn4C)c3)cnc2c1]

Followed General procedure 1 to afford 15 (50.0 mg, 29%). ¹H NMR (300 MHz, DMSO-d₆) δ10.59 (s, 1H), 9.31 (d, J=2.4 Hz, 1H), 8.90 (s, 1H), 8.55 (s, 1H), 8.05 (d, J=8.4 Hz, 1H), 7.91 (s, 1H), 7.81 (s, 1H), 7.75 (s, 1H), 7.56 (d, J=8.4 Hz, 1H), 7.32 (t, J=7.8 Hz, 1H), 7.05 (d, J=8.4 Hz, 1H), 4.69 (d, J=6.6 Hz, 1H), 3.40 (s, 3H), 2.58 (s, 3H), 1.67 (d, J=6.9 Hz, 3H). MS (ESI) calculated for (C₂₂H₂₁N₅OS) [M+H]⁺, 404.2; found, 404.3; found, 341.1.

### Example 16: 7-Chloro-N-(3-(1-((4-methyl-4H-1,2,4-triazol-3-yl)thio)ethyl)phenyl)quinoline-2-carboxamide (16)

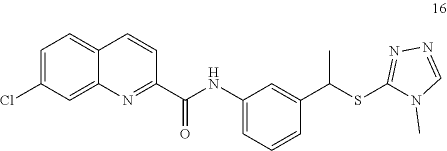 [SMILES: CC(Sc1nncn1C)c1cccc(NC(=O)c2ccc3ccc(Cl)cc3n2)c1]

Followed General procedure 1 to afford 16 (90.6 mg, 50%). ¹H NMR (300 MHz, DMSO-d₆) δ 10.78 (s, 1H), 8.71 (d, J=8.4 Hz, 1H), 8.56 (s, 1H), 8.35-8.16 (m, 3H), 7.98 (t, J=1.9 Hz, 1H), 7.95-7.77 (m, 2H), 7.35 (t, J=7.8 Hz, 1H), 7.07 (d, J=7.8 Hz, 1H), 4.71 (q, J=6.9 Hz, 1H), 3.41 (s, 3H), 1.70 (d, J=6.9 Hz, 3H). MS (ESI) calculated for (C₂₁H₁₈ClN₅OS) [M+H]⁺, 424.1; found, 424.2.

### Example 17: N-(3-(1-((4-Methyl-4H-1,2,4-triazol-3-yl)thio)ethyl)phenyl)quinoxaline-2-carboxamide (17)

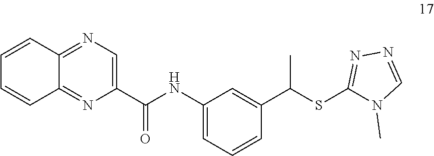 [SMILES: CC(Sc1nncn1C)c1cccc(NC(=O)c2cnc3ccccc3n2)c1]

Followed General procedure 1 to afford 17 (59.2 mg, 36%). ¹H NMR (400 MHz, DMSO-d₆) δ 10.87 (s, 1H), 9.57 (s, 1H), 8.56 (s, 1H), 8.37-8.19 (m, 2H), 8.08-7.96 (m, 3H), 7.94-7.83 (m, 1H), 7.35 (t, J=8.0 Hz, 1H), 7.08 (d, J=8.0 Hz, 1H), 4.71 (q, J=6.8 Hz, 1H), 3.41 (s, 3H), 1.69 (d, J=6.8 Hz, 3H). MS (ESI) calc'd for (C₂₀H₁₈N₆OS) [M+H]⁺, 391.1; found, 391.2.

### Example 18: N-(3-(1-((4-Methyl-4H-1,2,4-triazol-3-yl)thio)ethyl)phenyl)-1,8-naphthyridine-2-carboxamide (18)

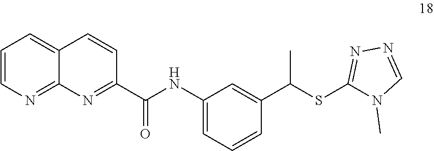 [SMILES: CC(Sc1nncn1C)c1cccc(NC(=O)c2ccc3cccnc3n2)c1]

Followed General procedure 1 to afford 18 (80.0 mg, 48%). ¹H NMR (400 MHz, DMSO-d₆) δ 10.85 (s, 1H), 9.27-9.26 (m, 1H), 8.77 (d, J=8.4 Hz, 1H), 8.64 (d, J=8.0 Hz, 1H), 8.57 (s, 1H), 8.37 (d, J=8.4 Hz, 1H), 8.02 (s, 1H), 7.94-7.84 (m, 1H), 7.80 (d, J=8.0 Hz, 1H), 7.34 (t, J=8.0 Hz, 1H), 7.07 (d, J=8.0 Hz, 1H), 4.70 (q, J=6.8 Hz, 1H), 3.41 (s, 3H), 1.70 (d, J=6.8 Hz, 3H). MS (ESI) calc'd for (C₂₀H₁₈N₆OS) [M+H]⁺, 391.1; found, 391.2.

### Example 19: N-(3-(1-((4-Methyl-4H-1,2,4-triazol-3-yl)thio)ethyl)phenyl)-2,7-naphthyridine-3-carboxamide (19)

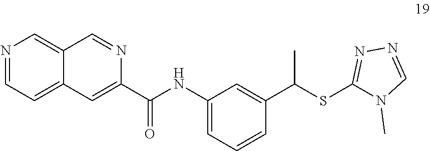 [SMILES: CC(Sc1nncn1C)c1cccc(NC(=O)c2cc3ccncc3cn2)c1]

Followed General procedure 1 to afford 19 (80.3 mg, 48%). ¹H NMR (300 MHz, DMSO-d₆) δ 10.87 (s, 1H), 9.73-9.71 (m, 2H), 8.90 (d, J=5.7 Hz, 1H), 8.74 (s, 1H), 8.57 (s, 1H), 8.23-8.12 (m, 1H), 8.01 (s, 1H), 7.97-7.88 (m, 1H), 7.34 (t, J=7.8 Hz, 1H), 7.08 (d, J=7.8 Hz, 1H), 4.70 (q, J=6.9 Hz, 1H), 3.42 (s, 3H), 1.70 (d, J=7.0 Hz, 3H). MS (ESI) calc'd for (C₂₀H₁₈N₆OS) [M+H]⁺, 391.1; found, 391.3.

### Example 20: N-(3-(1-((4-Methyl-4H-1,2,4-triazol-3-yl)thio)ethyl)phenyl)-1,5-naphthyridine-2-carboxamide (20)

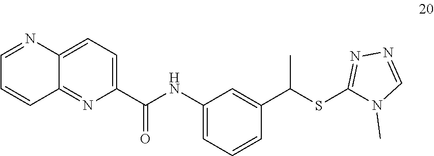 [SMILES: CC(Sc1nncn1C)c1cccc(NC(=O)c2ccc3ncccc3n2)c1]

Followed General procedure 1 to afford 20 (80.7 mg, 48%). ¹H NMR (400 MHz, DMSO-d₆) δ 10.82 (s, 1H), 9.17-9.15 (m, 1H), 8.73-8.59 (m, 2H), 8.56 (s, 1H), 8.48 (d, J=8.8 Hz, 1H), 8.03-7.93 (m, 2H), 7.93-7.86 (m, 1H), 7.35 (t, J=8.0 Hz, 1H), 7.08 (d, J=8.0 Hz, 1H), 4.71 (q, J=6.8 Hz, 1H), 3.41 (s, 3H), 1.70 (d, J=6.8 Hz, 3H). MS (ESI) calc'd for (C₂₀H₁₈N₆OS) [M+H]⁺, 391.1; found, 391.1.

### Example 21: 7-Methyl-N-(3-(1-((4-methyl-4H-1,2,4-triazol-3-yl)thio)ethyl)phenyl)-1,8-naphthyridine-2-carboxamide (21)

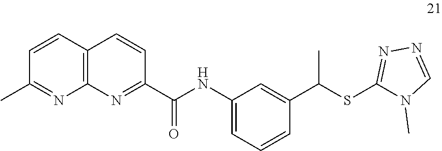 [SMILES: Cc1ccc2ccc(C(=O)Nc3cccc(C(C)Sc4nncn4C)c3)nc2n1]

Followed General procedure 1 to afford 21 (68.0 mg, 39%). ¹H NMR (300 MHz, DMSO-d₆) δ 10.82 (s, 1H), 8.70 (d, J=8.4 Hz, 1H), 8.62-8.46 (m, 2H), 8.30 (d, J=8.4 Hz, 1H), 8.02 (t, J=1.8 Hz, 1H), 8.01-7.91 (m, 1H), 7.69 (d, J=8.4 Hz, 1H), 7.33 (t, J=7.8 Hz, 1H), 7.12-7.02 (m, 1H), 4.70 (q, J=6.9 Hz, 1H), 3.41 (s, 3H), 2.79 (s, 3H), 1.69 (d, J=6.9 Hz, 3H). MS (ESI) calculated for (C₂₁H₂₀N₆OS) [M+H]⁺, 405.1; found, 405.1.

### Example 22: N-(3-(1-((4-Methyl-4H-1,2,4-triazol-3-yl)thio)ethyl)phenyl)-1-oxo-1,2-dihydroisoquinoline-3-carboxamide (22)

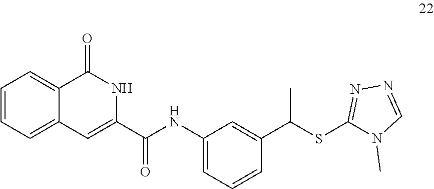 [SMILES: CC(Sc1nncn1C)c1cccc(NC(=O)c2cc3ccccc3c(=O)[nH]2)c1]

Followed General procedure 1 to afford 22 (62.0 mg, 36%). ¹H NMR (300 MHz, DMSO-d₆) δ 10.99 (s, 1H), 10.56 (s, 1H), 8.56 (s, 1H), 8.32-8.23 (m, 1H), 7.88-7.59 (m, 5H), 7.47 (s, 1H), 7.42-7.33 (m, 1H), 7.07 (d, J=7.8 Hz, 1H), 4.75-4.69 (m, 1H), 3.40 (s, 3H), 1.67 (d, J=7.0 Hz, 3H). MS (ESI) calculated for (C₂₁H₁₉N₅O₂S) [M+H]⁺, 406.1; found, 406.1.

### Example 23: N-(3-(1-((4-Methyl-4H-1,2,4-triazol-3-yl)thio)ethyl)phenyl)-5,6,7,8-tetrahydroquinoline-2-carboxamide (23)

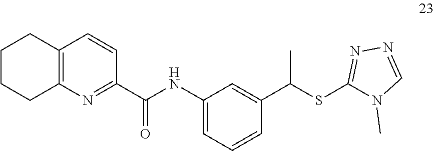 [SMILES: CC(Sc1nncn1C)c1cccc(NC(=O)c2ccc3c(n2)CCCC3)c1]

Followed General procedure 1 to afford 23 (97.8 mg, 58%). ¹H NMR (300 MHz, DMSO-d₆) δ 10.36 (s, 1H), 8.55 (s, 1H), 7.93-7.77 (m, 3H), 7.73 (d, J=7.8 Hz, 1H), 7.30 (t, J=7.8 Hz, 1H), 7.08-6.98 (m, 1H), 4.68 (q, J=6.9 Hz, 1H), 3.39 (s, 3H), 2.99 (t, J=6.3 Hz, 2H), 2.86 (t, J=6.3 Hz, 2H), 1.91-1.79 (m, 4H), 1.67 (d, J=6.9 Hz, 3H). MS (ESI) calculated for (C₂₁H₂₃N₅OS) [M+H]⁺, 394.2; found, 394.3.

### Example 24: N-(3-(1-((4-Methyl-4H-1,2,4-triazol-3-yl)thio)ethyl)phenyl)-5,6,7,8-tetrahydroisoquinoline-3-carboxamide (24)

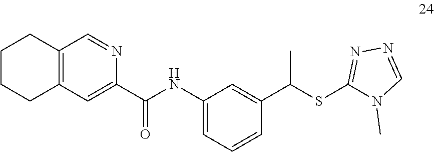 [SMILES: CC(Sc1nncn1C)c1cccc(NC(=O)c2cc3c(cn2)CCCC3)c1]

Followed General procedure 1 to afford 24 (96.8 mg, 58%). ¹H NMR (300 MHz, DMSO-d₆) δ 10.54 (s, 1H), 8.55 (s, 1H), 8.42 (s, 1H), 7.94 (s, 1H), 7.87-7.82 (m, 2H), 7.29 (t, J=7.8 Hz, 1H), 7.02 (d, J=7.8 Hz, 1H), 4.66 (q, J=6.9 Hz, 1H), 3.40 (s, 3H), 2.86-2.74 (m, 4H), 1.81-1.79 (m, 4H), 1.66 (d, J=6.9 Hz, 3H). MS (ESI) calculated for (C₂₁H₂₃N₅OS) [M+H]⁺, 394.2; found, 394.3.

### Example 25: 7-Chloro-N-(3-(1-((4-methyl-4H-1,2,4-triazol-3-yl)thio)ethyl)phenyl)isoquinoline-3-carboxamide (25)

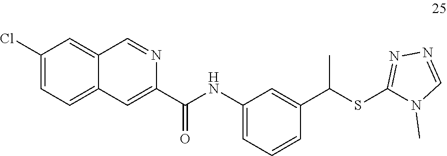 [SMILES: CC(Sc1nncn1C)c1cccc(NC(=O)c2cc3ccc(Cl)cc3cn2)c1]

Followed General procedure 1 to afford 25 (116.1 mg, 64%). ¹H NMR (300 MHz, DMSO-d₆) δ 10.77 (s, 1H), 9.48 (s, 1H), 8.75 (s, 1H), 8.56 (s, 1H), 8.48 (d, J=2.1 Hz, 1H), 8.33 (d, J=8.7 Hz, 1H), 7.99-7.88 (m, 3H), 7.32 (t, J=8.1 Hz, 1H), 7.05 (d, J=7.8 Hz, 1H), 4.69 (q, J=12 Hz, 1H), 3.41 (s, 3H), 1.69 (d, J=6.9 Hz, 3H). MS (ESI) calculated for (C₂₁H₁₈ClN₅OS) [M+H]⁺, 424.1; found, 424.3.

### Example 26: (S)-3-(Dimethylamino)-N-(3-(1-((4-methyl-4H-1,2,4-triazol-3-yl)thio)ethyl)phenyl)benzamide (26)

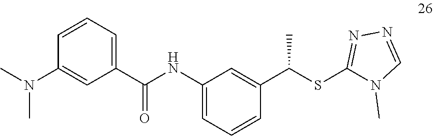 [SMILES: C[C@H](Sc1nncn1C)c1cccc(NC(=O)c2cccc(N(C)C)c2)c1]

Followed General procedure 2 to afford 26 (56.0 mg, 34%). ¹H NMR (400 MHz, DMSO-d₆) δ 10.16 (s, 1H), 8.55 (s, 1H), 7.82-7.80 (m, 1H), 7.71-7.69 (m, 1H), 7.38-7.21 (m, 4H), 7.02-7.01 (m, 1H), 6.95-6.89 (m, 1H), 4.69-4.64 (m, 1H), 3.40 (s, 3H), 2.98 (s, 6H), 1.66 (d, J=6.8 Hz, 3H). MS (ESI) calculated for (C₂₀H₂₃N₅OS) [M+H]⁺, 382.2; found, 382.2.

### Example 27: (S)-3,4-Dimethyl-N-(3-(1-((4-methyl-4H-1,2,4-triazol-3-yl)thio)ethyl)phenyl)benzamide (27)

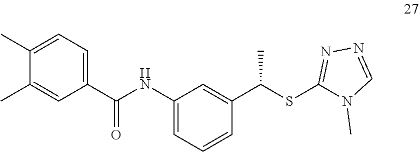 [SMILES: Cc1ccc(C(=O)Nc2cccc([C@H](C)Sc3nncn3C)c2)cc1C]

Followed General procedure 2 to afford 27 (60.7 mg, 39%). ¹H NMR (400 MHz, DMSO-d₆) δ 10.13 (s, 1H), 8.56 (s, 1H), 7.81-7.69 (m, 4H), 7.30-7.25 (m, 2H), 7.00 (d, J=6 Hz, 1H), 4.67 (q, J=6.8 Hz, 1H), 3.40 (s, 3H), 2.31 (s, 6H), 1.66 (d, J=6.8 Hz, 3H). MS (ESI) calculated for (C₂₀H₂₂N₄OS) [M+H]⁺, 367.2; found, 367.0.

### Example 28: (S)-4-Chloro-3-methyl-N-(3-(1-((4-methyl-4H-1,2,4-triazol-3-yl)thio)ethyl)phenyl)benzamide (28)

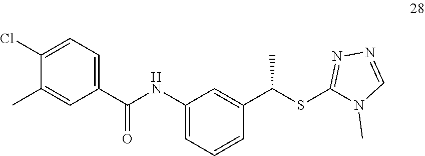 [SMILES: Cc1cc(C(=O)Nc2cccc([C@H](C)Sc3nncn3C)c2)ccc1Cl]

Followed General procedure 2 to afford 28 (58.8 mg, 36%). ¹H NMR (300 MHz, DMSO-d₆) δ 10.29 (s, 1H), 8.56 (s, 1H), 7.96 (s, 1H), 7.82-7.71 (m, 3H), 7.59 (d, J=8.4 Hz, 1H), 7.29 (t, J=7.9 Hz, 1H), 7.03 (d, J=7.5 Hz, 1H), 4.67 (q, J=6.9 Hz, 1H), 3.40 (s, 3H), 2.43 (s, 3H), 1.66 (d, J=6.9 Hz, 3H). MS (ESI) calculated for (C₁₉H₁₉ClN₄OS) [M+H]⁺, 387.1; found, 387.0.

### Example 29: (S)-N-(3-(1-((4-Methyl-4H-1,2,4-triazol-3-yl)thio)ethyl)phenyl)-3-(trifluoromethyl)benzamide (29)

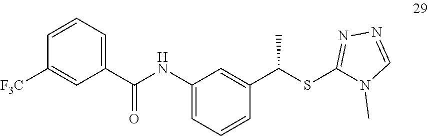 [SMILES: C[C@H](Sc1nncn1C)c1cccc(NC(=O)c2cccc(C(F)(F)F)c2)c1]

Followed General procedure 2 to afford 29 (85.6 mg, 49%). ¹H NMR (300 MHz, DMSO-d₆) δ 10.50 (s, 1H), 8.56 (s, 1H), 8.17-8.15 (m, 2H), 7.93 (d, J=8.1 Hz, 2H), 7.79 (s, 1H), 7.73 (d, J=8.1 Hz, 1H), 7.31 (t, J=7.8 Hz, 1H), 7.06 (d, J=7.8 Hz, 1H), 4.69 (q, J=6.9 Hz, 1H), 3.40 (s, 3H), 1.66 (d, J=6.9 Hz, 3H). MS (ESI) calculated for (C₁₉H₁₇F₃N₄OS) [M+H]⁺, 407.1; found, 407.0.

### Example 30: (S)-4-Methyl-N-(3-(1-((4-methyl-4H-1,2,4-triazol-3-yl)thio)ethyl)phenyl)quinoline-2-carboxamide (30)

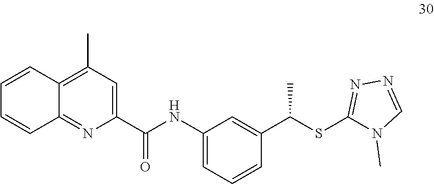 [SMILES: Cc1cc(C(=O)Nc2cccc([C@H](C)Sc3nncn3C)c2)nc2ccccc12]

Followed General procedure 2 to afford 30 (54.2 mg, 29%). ¹H NMR (300 MHz, DMSO-d₆) δ 10.75 (s, 1H), 8.55 (s, 1H), 8.29-8.22 (m, 2H), 8.13 (s, 1H), 7.98-7.90 (m, 3H), 7.79 (t, J=7.8 Hz, 1H), 7.34 (t, J=7.8 Hz, 1H), 7.06 (d, J=7.8 Hz, 1H), 4.70 (d, J=6.9 Hz, 1H), 3.41 (s, 3H), 2.83 (s, 3H), 1.70 (d, J=6.9 Hz, 3H). MS (ESI) calculated for (C₂₂H₂₁N₅OS) [M+H]⁺, 404.2; found, 404.0.

### Example 31: (S)-3-Isopropyl-N-(3-(1-((4-methyl-4H-1,2,4-triazol-3-yl)thio)ethyl)phenyl)benzamide (31)

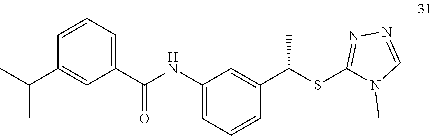 [SMILES: CC(C)c1cccc(C(=O)Nc2cccc([C@H](C)Sc3nncn3C)c2)c1]

Followed General procedure 2 to afford 31 (46.9 mg, 29%). ¹H NMR (300 MHz, DMSO-d₆) δ 10.23 (s, 1H), 8.55 (s, 1H), 7.89-7.77 (m, 3H), 7.73 (dt, J₁=8.1 Hz, J₂=1.5 Hz, 1H), 7.55-7.41 (m, 2H), 7.30 (t, J=7.8 Hz, 1H), 7.08-6.98 (m, 1H), 4.68 (q, J=6.9 Hz, 1H), 3.41 (s, 3H), 3.01 (p, J=6.8 Hz, 1H), 1.67 (d, J=6.9 Hz, 3H), 1.28 (d, J=6.9 Hz, 6H). MS (ESI) calculated for (C₂₁H₂₄N₄OS) [M+H]⁺, 381.2; found, 381.3.

### Example 32: (S)-N-(3-(1-((4-Methyl-4H-1,2,4-triazol-3-yl)thio)ethyl)phenyl)-4-(trifluoromethyl)picolinamide (32)

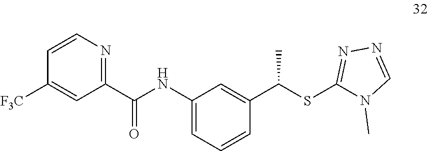 [SMILES: C[C@H](Sc1nncn1C)c1cccc(NC(=O)c2cc(C(F)(F)F)ccn2)c1]

Followed General procedure 2 to afford 32 (90.2 mg, 52%). ¹H NMR (300 MHz, DMSO-d₆) δ 10.81 (s, 1H), 9.04 (d, J=5.1 Hz, 1H), 8.55 (s, 1H), 8.38 (s, 1H), 8.11-8.09 (m, 1H), 7.97 (s, 1H), 7.85-7.82 (m, 1H), 7.33 (t, J=7.8 Hz, 1H), 7.06 (d, J=7.8 Hz, 1H), 4.67 (q, J=6.9 Hz, 1H), 3.39 (s, 3H), 1.66 (d, J=6.9 Hz, 3H). MS (ESI) calculated for (C₁₈H₁₆F₃N₅OS) [M+H]⁺, 408.1; found, 408.2.

### Example 33: (S)-6-Isopropyl-N-(3-(1-((4-methyl-4H-1,2,4-triazol-3-yl)thio)ethyl)phenyl)picolinamide (33)

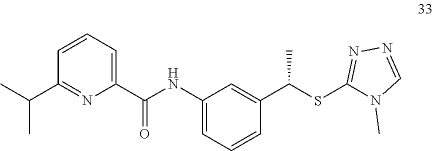 [SMILES: CC(C)c1cccc(C(=O)Nc2cccc([C@H](C)Sc3nncn3C)c2)n1]

Followed General procedure 2 to afford 33 (79.2 mg, 49%). ¹H NMR (300 MHz, DMSO-d₆) δ 10.34 (s, 1H), 8.58 (s, 1H), 8.02-7.96 (m, 2H), 7.86 (s, 1H), 7.80 (d, J=8.1 Hz, 1H), 7.60-7.57 (m, 1H), 7.33 (t, J=7.8 Hz, 1H), 7.05 (d, J=7.8 Hz, 1H), 4.70 (q, J=6.9 Hz, 1H), 3.39 (s, 3H), 3.25-3.16 (m, 1H), 1.68 (d, J=6.9 Hz, 3H), 1.35 (d, J=6.9 Hz, 6H). MS (ESI) calculated for (C₂₀H₂₃N₅OS) [M+H]⁺, 382.2; found, 382.3.

### Example 34: (S)-5-Chloro-4-methyl-N-(3-(1-((4-methyl-4H-1,2,4-triazol-3-yl)thio)ethyl)phenyl)picolinamide (34)

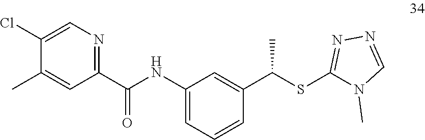 [SMILES: Cc1cc(C(=O)Nc2cccc([C@H](C)Sc3nncn3C)c2)ncc1Cl]

Followed General procedure 2 to afford 34 (41.4 mg, 35%) as a colorless solid. ¹H NMR (400 MHz, DMSO-d₆) δ 10.62 (s, 1H), 8.70 (s, 1H), 8.54 (s, 1H), 8.16 (s, 1H), 7.94 (s, 1H), 7.82 (d, J=8.0 Hz, 1H), 7.30 (t, J=8.0 Hz, 1H), 7.04 (d, J=7.6 Hz, 1H), 4.66 (q, J=7.0 Hz, 1H), 3.37 (s, 3H), 2.35 (s, 3H), 1.66 (d, J=12 Hz, 3H). MS (ESI) calculated for (C₁₈H₁₈ClN₅OS) [M+H]⁺, 388.1; found, 388.2.

### Example 35: (S)-5,6-Dimethyl-N-(3-(1-((4-methyl-4H-1,2,4-triazol-3-yl)thio)ethyl)phenyl)picolinamide (35)

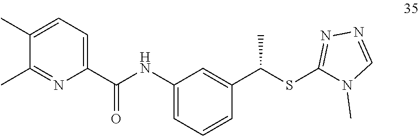 [SMILES: Cc1ccc(C(=O)Nc2cccc([C@H](C)Sc3nncn3C)c2)nc1C]

Followed General procedure 2 to afford 35 (93.8 mg, 60%) as a colorless solid. 1H NMR (400 MHz, DMSO-d₆) δ 10.41 (s, 1H), 8.55 (s, 1H), 7.91 (s, 2H), 7.84-7.77 (m, 2H), 7.30 (t, J=8.0 Hz, 1H), 7.03 (d, J=8.0 Hz, 1H), 4.68 (q, J=6.8 Hz, 1H), 3.40 (s, 3H), 2.60 (s, 3H), 2.37 (s, 3H), 1.68 (d, J=6.8 Hz, 3H). MS (ESI) calculated for (C₁₉H₂₁N₅OS) [M+H]⁺, 368.2; found, 368.3.

### Example 36: (S)-4,5-Dimethyl-N-(3-(1-((4-methyl-4H-1,2,4-triazol-3-yl)thio)ethyl)phenyl)picolinamide (36)

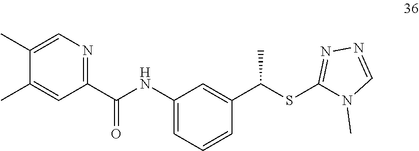 [SMILES: Cc1cnc(C(=O)Nc2cccc([C@H](C)Sc3nncn3C)c2)cc1C]

Followed General procedure 2 to afford 36 (29.8 mg, 19%) as a colorless solid. ¹H NMR (300 MHz, DMSO-d₆) δ 10.56 (s, 1H), 8.54 (s, 1H), 8.47 (s, 1H), 7.96-7.94 (m, 2H), 7.85-7.82 (m, 1H), 7.29 (t, J=7.8 Hz, 1H), 7.02 (d, J=7.8 Hz, 1H), 4.66 (q, J=6.9 Hz, 1H), 3.39 (s, 3H), 2.35 (s, 3H), 2.34 (s, 3H), 1.66 (d, J=6.9 Hz, 3H). MS (ESI) calculated for (C₁₉H₂₁N₅OS) [M+H]⁺, 368.2; found, 368.3.

### Example 37: (S)-4-Isopropyl-N-(3-(1-((4-methyl-4H-1,2,4-triazol-3-yl)thio)ethyl)phenyl)picolinamide (37)

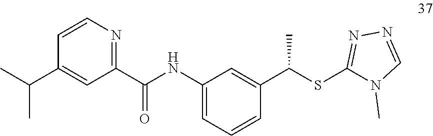 [SMILES: CC(C)c1ccnc(C(=O)Nc2cccc([C@H](C)Sc3nncn3C)c2)c1]

Followed General procedure 2 to afford 37 (23.8 mg, 12%) as a colorless solid. ¹H NMR (300 MHz, DMSO-d₆) δ 10.62 (s, 1H), 8.65-8.63 (m, 1H), 8.55 (s, 1H), 8.05 (s, 1H), 7.96 (s, 1H), 7.89-7.77 (m, 1H), 7.59 (d, J=5.1 Hz, 1H), 7.30 (t, J=7.8 Hz, 1H), 7.03 (d, J=7.2 Hz, 1H), 4.66 (q, J=6.9 Hz, 1H), 3.30 (s, 3H), 3.12-3.02 (m, 1H), 1.67 (d, J=6.9 Hz, 3H), 1.27 (d, J=6.9 Hz, 6H). MS (ESI) calculated for (C₂₀H₂₃N₅OS) [M+H]⁺, 382.2; found, 382.3.

### Example 38: (S)-6-Methoxy-N-(3-(1-((4-methyl-4H-1,2,4-triazol-3-yl)thio)ethyl)phenyl)picolinamide (38)

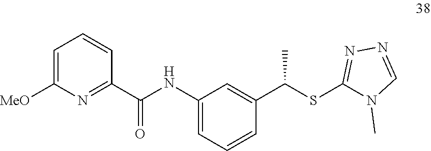 [SMILES: COc1cccc(C(=O)Nc2cccc([C@H](C)Sc3nncn3C)c2)n1]

Followed General procedure 2 to afford 38 (56.3 mg, 18%) as a colorless solid. ¹H NMR (300 MHz, DMSO-d₆) δ 10.18 (s, 1H), 8.54 (s, 1H), 7.97-7.92 (m, 1H), 7.84 (s, 1H), 7.78-7.72 (m, 2H), 7.35-7.29 (m, 1H), 7.12-7.04 (m, 2H), 4.72-4.65 (m, 1H), 4.08 (s, 3H), 3.33 (s, 3H), 1.67 (d, J=12 Hz, 3H). MS (ESI) calculated for (C₁₈H₁₉N₅O₂S) [M+H]⁺, 370.1; found, 370.3.

### Example 39: (S)-5-Chloro-6-methyl-N-(3-(1-((4-methyl-4H-1,2,4-triazol-3-yl)thio)ethyl)phenyl)picolinamide (39)

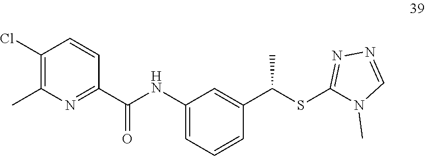 [SMILES: Cc1nc(C(=O)Nc2cccc([C@H](C)Sc3nncn3C)c2)ccc1Cl]

Followed General procedure 2 to afford 39 (72.9 mg, 18%) as a colorless solid. ¹H NMR (300 MHz, DMSO-d₆) δ 10.49 (s, 1H), 8.55 (s, 1H), 8.14 (d, J=8.3 Hz, 1H), 8.04-7.88 (m, 2H), 7.84 (d, J=8.1 Hz, 1H), 7.32 (t, J=7.8 Hz, 1H), 7.05 (d, J=7.8 Hz, 1H), 4.69 (q, J=6.9 Hz, 1H), 3.40 (s, 3H), 2.73 (s, 3H), 1.68 (d, J=6.9 Hz, 3H). MS (ESI) calculated for (C₁₈H₁₈ClN₅OS) [M+H]⁺, 388.1; found, 388.2.

### Example 40: (S)-5-Chloro-N-(3-(1-((4-methyl-4H-1,2,4-triazol-3-yl)thio)ethyl)phenyl)-4-(trifluoromethyl)picolinamide (40)

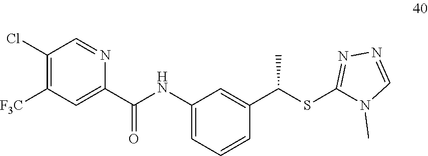 [SMILES: C[C@H](Sc1nncn1C)c1cccc(NC(=O)c2cc(C(F)(F)F)c(Cl)cn2)c1]

Followed General procedure 2 to afford 40 (21.1 mg, 11%) as a colorless solid. ¹H NMR (400 MHz, DMSO-d₆) δ 10.84 (s, 1H), 9.10 (s, 1H), 8.54 (s, 1H), 8.40 (s, 1H), 7.96-7.95 (m, 1H), 7.83-7.81 (m, 1H), 7.33-7.29 (m, 1H), 7.06 (d, J=8.0 Hz, 1H), 4.70-4.64 (m, 1H), 3.39 (s, 3H), 1.66 (d, J=6.8 Hz, 3H). MS (ESI) calculated for (C₁₈H₁₅ClF₃N₅OS) [M+H]⁺, 442.1; found, 442.2.

### Example 41: (S)-2-Methyl-N-(3-(1-((4-methyl-4H-1,2,4-triazol-3-yl)thio)ethyl)phenyl)thiazole-4-carboxamide (41)

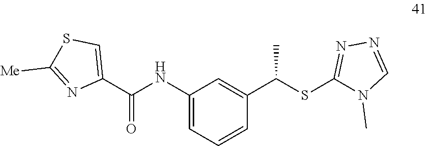 [SMILES: Cc1nc(C(=O)Nc2cccc([C@H](C)Sc3nncn3C)c2)cs1]

Followed General procedure 2 to afford 41 (57.6 mg, 53%) as a colorless solid. ¹H NMR (400 MHz, DMSO-d₆) δ 10.19 (s, 1H), 8.54 (s, 1H), 8.28 (s, 1H), 7.88 (t, J=2.0 Hz, 1H), 7.77-7.75 (m, 1H), 7.27 (t, J=8.0 Hz, 1H), 7.01-6.99 (m, 1H), 4.65 (q, J=6.8 Hz, 1H), 3.38 (s, 3H), 2.77 (s, 3H), 1.65 (d, J=6.8 Hz, 3H). MS (ESI) calculated for (C₁₆H₁₇N₅OS₂) [M+H]⁺, 360.0; found, 360.2.

### Example 42: (S)-2-Fluoro-N-(3-(1-((4-methyl-4H-1,2,4-triazol-3-yl)thio)ethyl)phenyl)-3-(trifluoromethyl)benzamide (42)

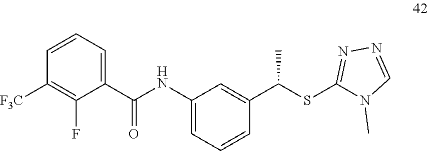 [SMILES: C[C@H](Sc1nncn1C)c1cccc(NC(=O)c2cccc(C(F)(F)F)c2F)c1]

Followed General procedure 2 to afford 42 (84.3 mg, 67%) as a colorless solid. ¹H NMR (400 MHz, DMSO-d₆) δ 10.67 (s, 1H), 8.55 (s, 1H), 8.05-7.92 (m, 2H), 7.70 (d, J=2.0 Hz, 1H), 7.65-7.63 (m, 1H), 7.56 (t, J=8.0 Hz, 1H), 7.31 (t, J=8.0 Hz, 1H), 7.14-7.02 (m, 1H), 4.68 (q, J=6.8 Hz, 1H), 3.41 (s, 3H), 1.65 (d, J=6.8 Hz, 3H). MS (ESI) calc'd for (C₁₉H₁₆F₄N₄OS) [M+H]⁺, 425.1; found, 425.1.

### Example 43: (S)-2-Methoxy-N-(3-(1-((4-methyl-4H-1,2,4-triazol-3-yl)thio)ethyl)phenyl)-3-(trifluoromethyl)benzamide (43)

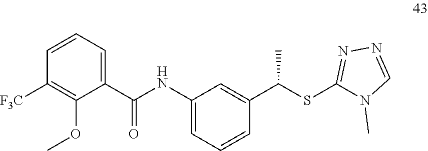 [SMILES: COc1c(C(=O)Nc2cccc([C@H](C)Sc3nncn3C)c2)cccc1C(F)(F)F]

Followed General procedure 2 to afford 43 (70.9 mg, 54%) as a colorless solid. ¹H NMR (400 MHz, DMSO-d₆) δ 10.55 (s, 1H), 8.54 (s, 1H), 7.82-7.81 (m, 2H), 7.71 (t, J=2.0 Hz, 1H), 7.69-7.59 (m, 1H), 7.41 (t, J=7.6 Hz, 1H), 7.31 (t, J=8.0 Hz, 1H), 7.11-7.02 (m, 1H), 4.68-4.65 (m, 1H), 3.84 (s, 3H), 3.39 (s, 3H), 1.65 (d, J=7.2 Hz, 3H). MS (ESI) calculated for (C₂₀H₁₉F₃N₄O₂S) [M+H]⁺, 437.1; found, 437.3.

### Example 44: (S)-N-(3-(1-((4-Methyl-4H-1,2,4-triazol-3-yl)thio)ethyl)phenyl)-3-(1H-pyrazol-5-yl)benzamide (44)

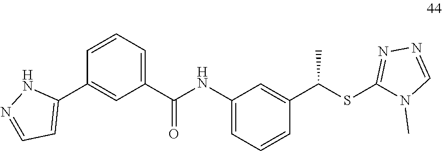 [SMILES: C[C@H](Sc1nncn1C)c1cccc(NC(=O)c2cccc(-c3ccn[nH]3)c2)c1]

Followed General procedure 2 to afford 44 (41.5 mg, 34%) as a colorless solid. ¹H NMR (400 MHz, DMSO-d₆) δ 13.00 (s, 1H), 10.37 (s, 1H), 8.55 (s, 1H), 8.38 (s, 1H), 8.03 (d, J=IF Hz, 1H), 7.91-7.79 (m, 3H), 7.77-7.70 (m, 1H), 7.58 (d, J=8.0 Hz, 1H), 7.30 (t, J=8.0 Hz, 1H), 7.03 (d, J=7.6 Hz, 1H), 6.83 (d, J=2.0 Hz, 1H), 4.68-4.65 (m, 1H), 3.41 (s, 3H), 1.67 (d, J=12 Hz, 3H). MS (ESI) calculated for (C₂₁H₂₀N₆OS) [M+H]⁺, 405.1; found, 405.1.

### Example 45: 3-(1-Methyl-1H-pyrazol-5-yl)-N-[3-[(1S)-1-[(4-methyl-4H-1,2,4-triazol-3-yl)sulfanyl]ethyl]phenyl]benzamide (45)

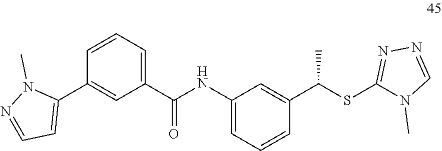 [SMILES: C[C@H](Sc1nncn1C)c1cccc(NC(=O)c2cccc(-c3ccnn3C)c2)c1]

Followed General procedure 2 to afford 45 (87.0 mg, 70%) as a colorless solid. ¹H NMR (400 MHz, DMSO-d₆) δ 10.35 (s, 1H), 8.55 (s, 1H), 8.09 (s, 1H), 8.02-8.00 (m, 1H), 7.81-7.71 (m, 3H), 7.67 (t, J=7.6 Hz, 1H), 7.53 (d, J=2.0 Hz, 1H), 7.30 (t, J=8.0 Hz, 1H), 7.05-7.03 (m, 1H), 6.53 (d, J=2.0 Hz, 1H), 4.68 (q, J=6.8 Hz, 1H), 3.91 (s, 3H), 3.40 (s, 3H), 1.66 (d, J=6.8 Hz, 3H). MS (ESI) calc'd for (C₂₂H₂₂N₆OS) [M+H]⁺, 419.2; found, 419.2.

### Example 46: (S)-4-(2-Hydroxyethyl)-N-(3-(1-((4-methyl-4H-1,2,4-triazol-3-yl)thio)ethyl)phenyl)picolinamide (46)

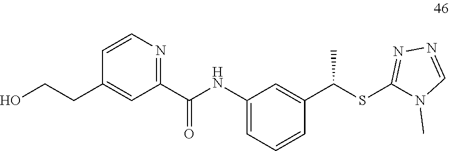 [SMILES: C[C@H](Sc1nncn1C)c1cccc(NC(=O)c2cc(CCO)ccn2)c1]

Followed General procedure 2 to afford 46 (58.8 mg, 51%) as a white semi-solid. ¹H NMR (300 MHz, DMSO-d₆) δ 10.62 (s, 1H), 8.62 (d, J=4.8 Hz, 1H), 8.54 (s, 1H), 8.05 (d, J=1.5 Hz, 1H), 7.95 (t, J=1.8 Hz, 1H), 7.85-7.83 (m, 1H), 7.56-7.55 (m, 1H), 7.30 (t, J=7.8 Hz, 1H), 7.03 (d, J=7.5 Hz, 1H), 4.78 (t, J=5.1 Hz, 1H), 4.66 (q, J=6.9 Hz, 1H), 3.72 (q, J=6.0 Hz, 2H), 3.39 (s, 3H), 2.88 (t, J=6.3 Hz, 2H), 1.67 (d, J=6.9 Hz, 3H). MS (ESI) calculated for (C₁₉H₂₁N₅O₂S) [M+H]⁺, 384.2; found, 384.2.

### Example 47: (S)-N-(3-(1-((4-Methyl-4H-1,2,4-triazol-3-yl)thio)ethyl)phenyl)-3-(methylamino)benzamide (47)

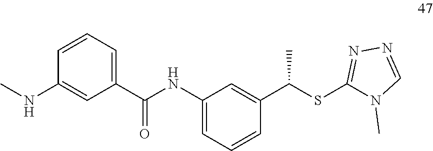 [SMILES: CNc1cccc(C(=O)Nc2cccc([C@H](C)Sc3nncn3C)c2)c1]

Followed General procedure 2 to afford 47 (20.6 mg, 19%) as an off-white solid, ¹H NMR (400 MHz, DMSO-d₆) δ 10.11 (s, 1H), 8.54 (s, 1H), 7.79 (t, J=2.0 Hz, 1H), 7.70 (dt, J=8.0, 1.2 Hz, 1H), 7.29-7.20 (m, 2H), 7.15-7.09 (m, 1H), 7.05 (t, J=2.1 Hz, 1H), 7.00 (d, J=7.6 Hz, 1H), 6.75-6.73 (m, 1H), 5.89 (q, J=5.2 Hz, 1H), 4.66 (q, J=6.8 Hz, 1H), 3.40 (s, 3H), 2.74 (d, J=5.2 Hz, 3H), 1.65 (d, J=6.8 Hz, 3H). MS (ESI) calc'd for (C₁₉H₂₁N₅OS) [M+H]⁺, 368.2; found, 368.2.

### Example 48: 5-Methyl-N-[3-[(1S)-1-[(4-methyl-4H-1,2,4-triazol-3-yl)sulfanyl]ethyl]phenyl]quinoline-2-carboxamide (48)

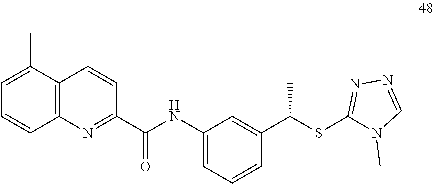 [SMILES: Cc1cccc2nc(C(=O)Nc3cccc([C@H](C)Sc4nncn4C)c3)ccc12]

Followed General procedure 2 to afford 48 (33.0 mg, 8%) as an off-white solid. ¹H NMR (300 MHz, DMSO-d₆) δ 10.77 (s, 1H), 8.76-8.73 (m, 1H), 8.55 (s, 1H), 8.27 (d, J=8.7 Hz, 1H), 8.13 (d, J=8.7 Hz, 1H), 8.00 (s, 1H), 7.93-7.90 (m, 1H), 7.84-7.81 (m, 1H), 7.62-7.60 (m, 1H), 7.34 (t, J=7.8 Hz, 1H), 7.11-7.01 (m, 1H), 4.74-4.70 (m, 1H), 3.41 (s, 3H), 2.74 (s, 3H), 1.70 (d, J=6.9 Hz, 3H). MS (ESI) calc'd for (C₂₂H₂₁N₅OS) [M+H]⁺, 404.1; found, 403.9.

### Example 49: (S)-3-Amino-N-(3-(1-((4-methyl-4H-1,2,4-triazol-3-yl)thio)ethyl)phenyl)-6-(trifluoromethyl)picolinamide (49)

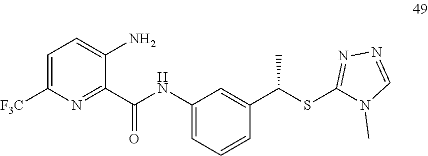 [SMILES: C[C@H](Sc1nncn1C)c1cccc(NC(=O)c2nc(C(F)(F)F)ccc2N)c1]

Followed General procedure 2 to afford 49 (106.6 mg, 84%) as a light yellow solid. ¹H NMR (400 MHz, DMSO-d₆) δ 10.08 (s, 1H), 8.54 (s, 1H), 7.80 (t, J=2.0 Hz, 1H), 7.77-7.64 (m, 2H), 7.51-7.36 (m, 3H), 7.30 (t, J=8.0 Hz, 1H), 7.08-6.99 (m, 1H), 4.68 (q, J=6.8 Hz, 1H), 3.39 (s, 3H), 1.67 (d, J=12 Hz, 3H). MS (ESI) calculated for (C₁₈H₁₇F₃N₆OS) [M+H]⁺, 423.2; found, 423.2.

### Example 50: (S)-4-Methyl-N-(3-(1-((4-methyl-4H-1,2,4-triazol-3-yl)thio)ethyl)phenyl)-6-oxo-1,6-dihydropyridine-2-carboxamide (50)

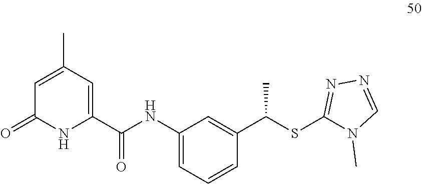 [SMILES: Cc1cc(C(=O)Nc2cccc([C@H](C)Sc3nncn3C)c2)[nH]c(=O)c1]

Followed General procedure 2 to afford 50 (23.9 mg, 22%) as a light yellow solid. ¹H NMR (400 MHz, DMSO-d₆) δ 11.13 (s, 1H), 10.22 (s, 1H), 8.53 (s, 1H), 7.85-7.65 (m, 2H), 7.30 (t, J=8.0 Hz, 2H), 7.16-6.93 (m, 1H), 6.63 (s, 1H), 4.67 (q, J=6.8 Hz, 1H), 3.38 (s, 3H), 2.31 (s, 3H), 1.65 (d, J=6.8 Hz, 3H). MS (ESI) calculated for (C₁₈H₁₉N₅O₂S) [M+H]⁺, 370.2; found, 370.2.

### Example 51: (S)-5-chloro-N-(3-(1-((4-methyl-4H-1,2,4-triazol-3-yl)thio)ethyl)phenyl)-6-(trifluoromethyl)picolinamide (51)

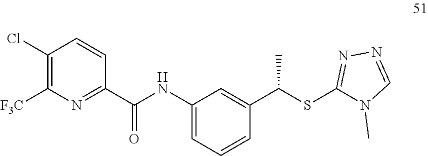 [SMILES: C[C@H](Sc1nncn1C)c1cccc(NC(=O)c2ccc(Cl)c(C(F)(F)F)n2)c1]

Followed General procedure 2 to afford 51 (49.8 mg, 26%) as a colorless solid. MS (ESI) calculated for (C₁₈H₁₅ClF₃N₅OS) [M+H]⁺, 442.1; found, 442.1. ¹H NMR (400 MHz, DMSO-d₆) δ 10.41 (s, 1H), 8.56-8.47 (m, 2H), 8.37 (d, J=8.4 Hz, 1H), 7.84 (s, 1H), 7.80-7.72 (m, 1H), 7.32 (t, J=8.0 Hz, 1H), 7.11-7.03 (m, 1H), 4.69 (q, J=6.8 Hz, 1H), 3.39 (s, 3H), 1.66 (d, J=6.8 Hz, 3H).

### Example 52: (S)-5-methyl-N-(3-(1-((4-methyl-4H-1,2,4-triazol-3-yl)thio)ethyl)phenyl)-4-(trifluoromethyl)picolinamide (52)

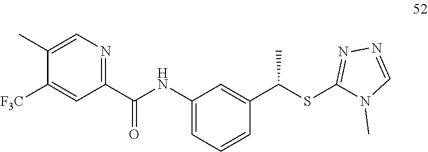 [SMILES: Cc1cnc(C(=O)Nc2cccc([C@H](C)Sc3nncn3C)c2)cc1C(F)(F)F]

Followed General procedure 2 to afford 52 (26.4 mg, 15%) as a colorless solid. MS (ESI) calculated for (C₁₉H₁₈F₃N₅OS) [M+H]⁺, 422.1; found, 421.9. ¹H NMR (300 MHz, DMSO-d₆) δ 10.76 (s, 1H), 8.89 (s, 1H), 8.54 (s, 1H), 8.28 (s, 1H), 7.97 (s, 1H), 7.84 (d, J=8.4 Hz, 1H), 7.30 (t, J=8.0 Hz, 1H), 7.05 (d, J=7.8 Hz, 1H), 4.68 (q, J=6.9 Hz, 1H), 3.40 (s, 3H), 2.58 (s, 3H), 1.66 (d, J=6.9 Hz, 3H).

### Example 53: (S)-4-cyclopropyl-5-methyl-N-(3-(1-((4-methyl-4H-1,2,4-triazol-3-yl)thio)ethyl)phenyl)picolinamide (53)

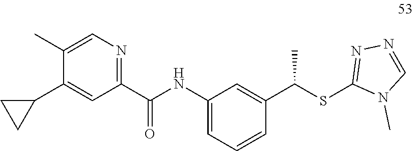 [SMILES: Cc1cnc(C(=O)Nc2cccc([C@H](C)Sc3nncn3C)c2)cc1C1CC1]

Followed General procedure 2 to afford 53 (9.6 mg, 6%) as a colorless solid. MS (ESI) calculated for (C₂₁H₂₃N₅OS) [M+H]+, 394.2; found, 394.3. ¹H NMR (300 MHz, DMSO-d₆) δ 10.55 (s, 1H), 8.55 (s, 1H), 8.46 (s, 1H), 7.96 (s, 1H), 7.81-7.78 (m, 1H), 7.58 (s, 1H), 7.27 (t, J=7.8 Hz, 1H), 7.01 (d, J=7.8 Hz, 1H), 4.69-4.62 (m, 1H), 3.38 (s, 3H), 2.48 (s, 3H), 2.11-2.06 (m, 1H), 1.66 (d, J=6.9 Hz, 3H), 1.16-1.09 (m, 2H), 0.84 (d, J=6.0 Hz, 2H).

### Example 54: (S)-6-cyclopropyl-5-methyl-N-(3-(1-((4-methyl-4H-1,2,4-triazol-3-yl)thio)ethyl)phenyl)picolinamide (54)

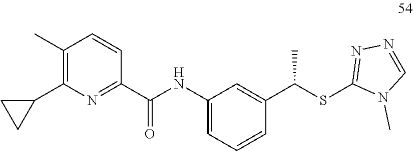 [SMILES: Cc1ccc(C(=O)Nc2cccc([C@H](C)Sc3nncn3C)c2)nc1C1CC1]

Followed General procedure 2 to afford 54 (40.4 mg, 24%) as a light yellow solid. MS (ESI) calc'd for (C₂₁H₂₃N₅OS) [M+H]⁺, 394.2; found, 394.2. ¹H NMR (300 MHz, Methanol-d₄) δ 8.43 (s, 1H), 7.84 (d, J=7.8 Hz, 1H), 7.73-7.64 (m, 2H), 7.61-7.57 (m, 1H), 7.29 (t, J=7.8 Hz, 1H), 7.03-7.04 (m, 1H), 4.67 (q, J=6.9 Hz, 1H), 3.39 (s, 3H), 2.52 (s, 3H), 2.29-2.26 (m, 1H), 1.75 (d, J=6.9 Hz, 3H), 1.22-1.17 (m, 2H), 1.18-1.02 (m, 2H).

### Example 55: (S)-5-chloro-4-(2-hydroxypropan-2-yl)-N-(3-(1-((4-methyl-4H-1,2,4-triazol-3-yl)thio)ethyl)phenyl)picolinamide (55)

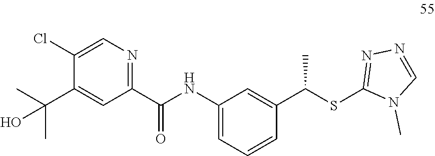 [SMILES: C[C@H](Sc1nncn1C)c1cccc(NC(=O)c2cc(C(C)(C)O)c(Cl)cn2)c1]

Followed General procedure 2 to afford 55 (139.5 mg, 70%) as a colorless solid. MS (ESI) calc'd for (C₂₀H₂₂ClN₅O₂S) [M+H]⁺, 432.1; found, 432.2. ¹H NMR (300 MHz, DMSO-d₆) δ 10.63 (s, 1H), 8.64 (s, 1H), 8.57 (s, 1H), 8.54 (s, 1H), 7.91 (s, 1H), 7.81 (d, J=8.1 Hz, 1H), 7.28 (t, J=7.8 Hz, 1H), 7.02 (d, J=7.8 Hz, 1H), 5.77 (br, 1H), 4.64 (q, J=6.9 Hz, 1H), 3.40 (s, 3H), 1.69-1.57 (m, 9H).

### Example 56: (S)-4,6-dimethyl-N-(3-(1-((4-methyl-4H-1,2,4-triazol-3-yl)thio)ethyl)phenyl)picolinamide (56)

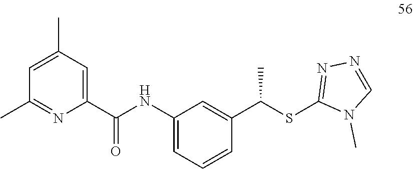 [SMILES: Cc1cc(C)nc(C(=O)Nc2cccc([C@H](C)Sc3nncn3C)c2)c1]

A-a (20 mg, 0.087 mmol) and 4,6-dimethylpicolinic acid (16 mg, 0.10 mmol) were dissolved in a mixture of EtOAc (0.50 mL) and N,N-dimethylformamide (0.10 mL) at rt. Propylphosphonic anhydride solution in EtOAc (0.090 mL, 1.5 M) was added followed by pyridine (35 μL, 0.43 mmol). The reaction was stirred overnight. 1 N hydrochloric acid and EtOAc were added to the mixture and the layers separated. The aqueous layer was extracted with chloroform:isopropyl alcohol (2:1) (2×). The combined organic layers were dried, and concentrated. The residue was purified by flash chromatography to afford 56 (3.8 mg, 12%): ¹H NMR (500 MHz, DMSO-d₆) δ 10.41 (s, 1H), 8.53 (s, 1H), 7.88 (t, J=1.9 Hz, 1H), 7.87-7.81 (m, 1H), 7.80 (d, J=1.5 Hz, 1H), 7.37 (s, 1H), 7.30 (t, J=7.9 Hz, 1H), 7.02 (d, J=7.7 Hz, 1H), 4.67 (q, J=6.9 Hz, 1H), 3.38 (s, 3H), 2.59 (s, 3H), 2.40 (s, 3H), 1.67 (d, J=7.0 Hz, 3H). LCMS: C₁₉H₂₁N₅OS requires: 367.5, found: m/z 368.4 [M+H]⁺.

### Example 57a: (S)-N-(3-(1-((4-methyl-4H-1,2,4-triazol-3-yl)thio)ethyl)phenyl)-6-(trifluoromethyl)picolinamide (57a)

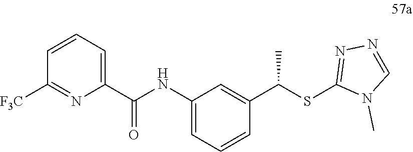 [SMILES: C[C@H](Sc1nncn1C)c1cccc(NC(=O)c2cccc(C(F)(F)F)n2)c1]

A solution of A-a (10.0 g, 42.7 mmol), pyridine (14.4 mL, 179 mmol) and 6-(trifluoromethyl)picolinic acid (10.1 g, 51.2 mmol) in EtOAc (210 mL) was purged with nitrogen, cooled to 0° C. and treated with propylphosphonic anhydride (50 wt. % solution in EtOAc, 30.5 mL, 51.2 mmol) dropwise. The mixture was warmed to rt and stirred for 16 h. The reaction was quenched with saturated aqueous sodium bicarbonate. General Work-up Procedure 1 was followed. The desired 57a (15.2 g, 87.1%) was obtained as an off-white solid using standard flash chromatography purification methods, ¹H NMR (500 MHz, DMSO-d6) δ 10.43 (s, 1H), 8.54 (s, 1H), 8.43-8.31 (m, 2H), 8.19 (dd, J=7.4, 1.4 Hz, 1H), 7.88 (t, J=2.0 Hz, 1H), 7.81 (dt, J=8.4, 1.2 Hz, 1H), 7.33 (t, J=7.9 Hz, 1H), 7.07 (dt, J=7.7, 1.3 Hz, 1H), 4.70 (q, J=7.0 Hz, 1H), 3.39 (s, 3H), 1.68 (d, J=7.0 Hz, 3H). LCMS: C₁₋₈H₁₆F₃N₅OS requires: 407, found: m/z=408 [M+H]⁺.

### Example 57b: (R)-N-(3-(1-((4-methyl-4H-1,2,4-triazol-3-yl)thio)ethyl)phenyl)-6-(trifluoromethyl)picolinamide (57b)

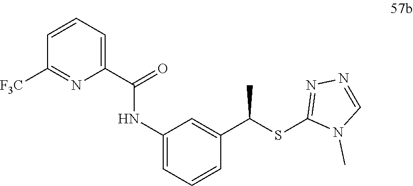 [SMILES: C[C@@H](Sc1nncn1C)c1cccc(NC(=O)c2cccc(C(F)(F)F)n2)c1]

Followed General procedure 3 to afford 57b (26 mg, 15%) as an off-white solid. MS (ESI) calculated for (C₁₈H₁₆F₃N₅OS) [M+H]⁺, 408.1; found, 407.9. ¹H NMR (300 MHz, DMSO-d₆) δ 10.44 (s, 1H), 8.54 (s, 1H), 8.42-8.34 (m, 2H), 8.20-8.17 (m, 1H), 7.88 (s, 1H) 7.81 (d, J=8.1 Hz, 1H), 7.35-7.30 (m, 1H), 7.06 (d, J=7.8 Hz, 1H), 4.73-4.66 (m, 1H), 3.40 (s, 3H), 1.67 (d, J=6.9 Hz, 3H).

### Example 58: (S)-3-amino-N-(3-(1-((4-methyl-4H-1,2,4-triazol-3-yl)thio)ethyl)phenyl)benzamide (58)

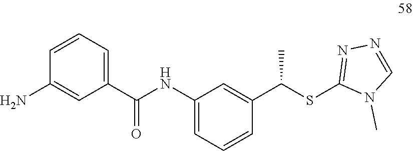 [SMILES: C[C@H](Sc1nncn1C)c1cccc(NC(=O)c2cccc(N)c2)c1]

Compound 58 was made according to procedures disclosed herein (21.3 mg, 27%) as a light yellow solid. MS (ESI) calc'd for (C₁₈H₁₉N₅OS) [M+H]⁺, 354.1; found, 354.2. ¹H NMR (400 MHz, DMSO-d₆) δ 10.56 (s, 1H), 9.41-9.18 (m, 1H), 8.04 (d, J=7.8 Hz, 1H), 7.92 (t, J=1.8 Hz, 1H), 7.85 (d, J=2.0 Hz, 1H), 7.74-7.70 (m, 1H), 7.68-7.58 (m, 2H), 7.33 (t, J=7.8 Hz, 1H), 7.20-6.99 (m, 1H), 4.84 (q, J=6.8 Hz, 1H), 3.53 (s, 3H), 1.70 (d, J=6.8 Hz, 3H).

### Example 59: (S)-4-acetamido-N-(3-(1-((4-methyl-4H-1,2,4-triazol-3-yl)thio)ethyl)phenyl)picolinamide (59)

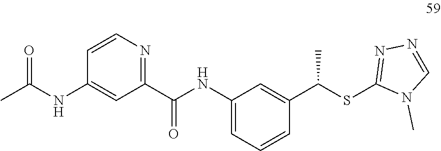 [SMILES: CC(=O)Nc1ccnc(C(=O)Nc2cccc([C@H](C)Sc3nncn3C)c2)c1]

Step 1: Synthesis of (S)-N-(3-(1-((4-methyl-4H-1,2,4-triazol-3-yl)thio)ethyl)phenyl)-4-nitropicolinamide. To a solution of A-a (200 mg, 0.85 mmol), 4-nitropicolinic acid (101 mg, 0.60 mmol) and N,N-diisopropylethylaminc (232 mg, 1.79 mmol) in N,N-dimethylformamide (2 mL) was added HATU (272 mg, 0.72 mmol). The resulting solution was stirred at room temperature for 2 h. The reaction was quenched by water. The aqueous solution was extracted with EtOAc. The combined organic layer was washed with brine, dried, filtered and concentrated under vacuum. The residue was purified by flash column chromatography to afford the title compound (120 mg, 36%) as a yellow oil.

Step 2: Synthesis of (S)-4-amino-N-(3-(1-((4-methyl-4H-1,2,4-triazol-3-yl)thio)ethyl)phenyl)picolinamide: To a solution of (S)-N-(3-(1-((4-methyl-4H-1,2,4-triazol-3-yl)thio)ethyl)phenyl)-4-nitropicolinamide (120 mg, 0.31 mmol) in ethanol (6 mL) and water (1 mL) was added iron powder (52 mg, 0.93 mmol) and ammonium chloride (99 mg, 1.86 mmol). The mixture was stirred at 80° C. for 16 h. The mixture was filtered and concentrated under reduced pressure to afford the title compound (100 mg, crude) as a yellow oil, which was used in the next step without purification.

Step 3: Synthesis of (S)-4-acetamido-N-(3-(1-((4-methyl-4H-1,2,4-triazol-3-yl)thio)ethyl)phenyl)picolinamide: To a stirred solution of (S)-4-amino-N-(3-(1-((4-methyl-4H-1,2,4-triazol-3-yl)thio)ethyl)phenyl)picolinamide (90 mg, 0.25 mmol) and pyridine (50 mg, 0.64 mmol) in dichloromethane (1 mL) was added acetyl chloride (30 mg, 0.38 mmol). The solution was stirred at room temperature for 1 h. The solution was concentrated under reduced pressure and purified by prep-HPLC to afford the title compound as a white solid(16.1 mg, 9%), MS (ESI) calculated for (C₁₉H₂₀N₆O₂S) [M+H]⁺, 396.1; found, 396.3. ¹H NMR (400 MHz, DMSO-d₆) δ 10.60 (s, 2H), 8.62-8.51 (m, 2H), 8.32 (d, J=2.0 Hz, 1H), 7.93 (t, J=2.0 Hz, 1H), 7.83-7.80 (m, 2H), 7.29 (t, J=8.0 Hz, 1H), 7.07-6.99 (m, 1H), 4.66 (q, J=6.8 Hz, 1H), 3.39 (s, 3H), 2.14 (s, 3H), 1.66 (d, J=7.2 Hz, 3H).

### Example 60: (S)-N-(3-(1-((4-methyl-4H-1,2,4-triazol-3-yl)thio)ethyl)phenyl)-4-(methylamino)picolinamide (60)

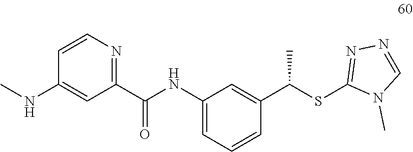 [SMILES: CNc1ccnc(C(=O)Nc2cccc([C@H](C)Sc3nncn3C)c2)c1]

Compound 60 (4 mg, 2%) was obtained according to procedures disclosed herein as a colorless solid. MS (ESI) calculated for (C₁₈H₂₀N₆OS) [M+H]⁺, 368.1; found, 369.2. ¹H NMR (300 MHz, DMSO-d₆) δ 10.48 (s, 1H), 8.54 (s, 1H), 8.18 (d, J=5.7 Hz, 1H), 7.92 (s, 1H), 7.81-7.78 (m, 1H), 7.31-7.25 (m, 2H), 7.01 (d, J=6.0 Hz, 2H), 6.68-6.65 (m, 1H), 4.65 (q, J=6.9 Hz, 1H), 3.39 (s, 3H), 2.78 (d, J=4.8 Hz, 3H), 1.66 (d, J=6.9 Hz, 3H).

### Example 61: (S)-5-methyl-N-(3-(1-((4-methyl-4H-1,2,4-triazol-3-yl)thio)ethyl)phenyl)-6-(trifluoromethyl)picolinamide (61)

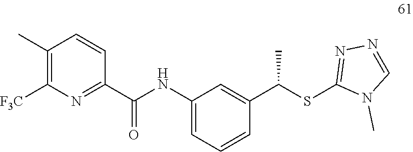 [SMILES: Cc1ccc(C(=O)Nc2cccc([C@H](C)Sc3nncn3C)c2)nc1C(F)(F)F]

To a degassed solution of Example 51 (80 mg, 0.18 mmol) in anhydrous THF (1 mL) were added methylboronic acid (65 mg, 1.09 mmol), chloro(2-dicyclohexylphosphino-2′,6′-dimethoxy-1,1′-biphenyl)[2-(2′-amino-1,1′-biphenyl)]palladium(II) (20 mg, 0.03 mmol) and KF (31.6 mg, 0.54 mmol) at rt. Then the mixture was heated at 80° C. for 16 h, and then concentrated. The desired 61 (10.3 mg, 13%) was obtained as a colorless solid using standard HPLC purification methods. MS (ESI) calc'd for (C₁₉H₁₈F₃N₅OS) [M+H]⁺, 422.2; found, 422.2. ¹H NMR (300 MHz, DMSO-d₆) δ 10.28 (s, 1H), 8.52 (s, 1H), 8.27 (d, J=8.1 Hz, 1H), 8.18 (d, J=8.1 Hz, 1H), 7.85 (d, J=1.5 Hz, 1H), 7.79-7.76 (m, 1H), 7.30 (t, J=7.8 Hz, 1H), 7.03 (d, J=7.8 Hz, 1H), 4.67 (q, J=6.9 Hz, 1H), 3.37 (s, 3H), 2.57 (s, 3H), 1.65 (d, J=6.9 Hz, 3H).

### Example 62: (S)-1-methoxy-N-(3-(1-((4-methyl-4H-1,2,4-triazol-3-yl)thio)ethyl)phenyl)isoquinoline-3-carboxamide (62)

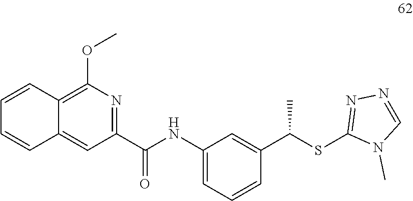 [SMILES: COc1nc(C(=O)Nc2cccc([C@H](C)Sc3nncn3C)c2)cc2ccccc12]

A mixture of A-a (0.0240 g, 0.102 mmol), triethylamine (0.042 mL, 0.030 mmol), 1-methoxyisoquinoline-3-carboxylic acid (0.0250 g, 0.123 mmol), and EDC (0.0582 g, 0.303 mmol) and 4-(dimethylamino)pyridine (0.0019 g, 0.015 mmol) in dichloromethane (0.47 mL) was stirred at rt for 14 h. The desired 62 (0.033 g, 76%). was obtained as an off-white powder using standard flash chromatography purification methods. Yield: 1H NMR (500 MHz, DMSO-d₆) δ 10.26 (s, 1H), 8.57 (s, 1H), 8.32-8.25 (m, 2H), 8.17 (d, J=8.1 Hz, 1H), 7.94-7.85 (m, 2H), 7.85-7.75 (m, 2H), 7.35 (t, J=7.8 Hz, 1H), 7.11-7.05 (m, 1H), 4.71 (p, J=6.6 Hz, 1H), 4.32 (s, 3H), 3.41 (s, 3H), 1.70 (d, J=7.0 Hz, 3H). LCMS: C₂₂H₂₁N₅O₂S requires: 419, found: m/z=420 [M+H]⁺.

### Example 63: (S)-N-(3-(1-((4-methyl-4H-1,2,4-triazol-3-yl)thio)ethyl)phenyl)-1-(trifluoromethyl)isoquinoline-3-carboxamide (63)

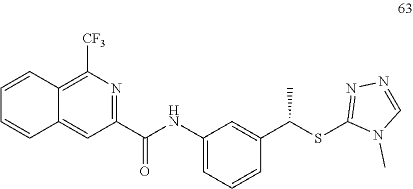 [SMILES: C[C@H](Sc1nncn1C)c1cccc(NC(=O)c2cc3ccccc3c(C(F)(F)F)n2)c1]

Step 1: Methyl 2-(2,2,2-trifluoroacetamido)acrylate: A solution of methyl 2-(chloroamino)-3-hydroxy-propanoate (5.00 g, 32.1 mmol) in dichloromethane (80.4 mL) was treated with a mixture of triethylamine (18.0 mL, 128 mmol) and trifluoroacetic anhydride (11.3 mL, 80.4 mmol) at 0° C. The reaction mixture was stirred at room temperature for 24 h. The mixture was diluted with water and extracted with EtOAc twice. The combined organic layers were washed with saturated sodium bicarbonate, brine, dried, filtered and concentrated in vacuo. The residue was purified by flash chromatography to give the title compound as a yellow oil. Yield: 0.300 g (4.72%).

Step 2: Methyl 1-(trifluoromethyl)isoquinoline-3-carboxylate: A solution of tetrabutylammonium difluorotriphenylsilicate (1.64 g, 3.04 mmol) and methyl 2-(2,2,2-trifluoroacetamido)acrylate (0.300 g, 1.52 mmol) in tetrahydrofuran (76.0 mL) was treated with 2-(trimethylsilyl)phenyl trifluoromethanesulfonate (0.738 mL, 3.04 mmol) at room temperature. The reaction mixture was stirred at room temperature for 16 h. The mixture was concentrated in vacuo. The residue was purified by flash chromatography to give the title compound as a yellow powder. Yield: 0.0140 g (3.61%).

Step 3: (S)-N-(3-(1-((4-methyl-4H-1,2,4-triazol-3-yl)thio)ethyl)phenyl)-1-(trifluoromethyl)isoquinoline-3-carboxamide: A suspension of A-a (0.0129 g, 0.0549 mmol) in tetrahydrofuran (0.20 mL) was cooled to 0° C. and treated with a solution of trimethylaluminum (2 M in toluene, 0.27 mL, 0.55 mmol) dropwise. After the addition was complete the solution was stirred at room temperature for 15 min. The mixture was added dropwise to a solution of methyl 1-(trifluoromethyl)isoquinoline-3-carboxylate (0.0140 g, 0.0549 mmol) in tetrahydrofuran (0.20 mL). The resulting solution was stirred at 40° C. for 16 h. The reaction was quenched with saturated aqueous Rochelle's salt and stirred for 1 h. The reaction mixture was extracted twice with EtOAc, washed with brine, dried (sodium sulfate), filtered and evaporated to dryness. Purification by flash chromatography afforded the title compound as an off-white solid. Yield: 0.0084 g (33%). ¹H NMR (500 MHz, DMSO-d6) δ 10.91 (s, 1H), 8.58 (s, 1H), 8.51 (s, 1H), 8.47 (dd, J=8.6, 1.2 Hz, 1H), 8.24 (d, J=8.5 Hz, 1H), 8.12 (ddd, J=8.3, 6.9, 1.3 Hz, 1H), 8.04-7.98 (m, 2H), 7.95-7.87 (m, 1H), 7.36 (t, J=7.9 Hz, 1H), 7.09 (dt, J=7.7, 1.3 Hz, 1H), 4.72 (q, J=7.0 Hz, 1H), 3.41 (s, 3H), 1.70 (d, J=7.0 Hz, 3H). LCMS: C₂₂H₁₈F₃N₅OS requires: 457, found: m/z=458 [M+H]⁺.

### Example 64: (S)-N-(3-(1-((4-methyl-4H-1,2,4-triazol-3-yl)thio)ethyl)phenyl)-4,6-bis(trifluoromethyl)picolinamide (64)

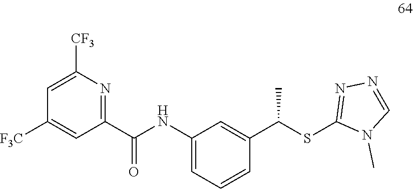 [SMILES: C[C@H](Sc1nncn1C)c1cccc(NC(=O)c2cc(C(F)(F)F)cc(C(F)(F)F)n2)c1]

Followed Example 57a to afford 64 (0.082 g, 81%) as an off-white solid. ¹H NMR (500 MHz, DMSO-d6) δ 10.65 (s, 1H), 8.63 (d, J=1.6 Hz, 2H), 8.56 (s, 1H), 7.87 (t, J=2.0 Hz, 1H), 7.82 (dt, J=8.3, 1.1 Hz, 1H), 7.34 (t, J=7.9 Hz, 1H), 7.10 (dt, J=7.7, 1.3 Hz, 1H), 4.71 (q, J=6.9 Hz, 1H), 3.40 (s, 3H), 1.68 (d, J=7.0 Hz, 3H). LCMS: C₁₉H₁₅F₆N₅OS requires: 475, found: m/z=476 [M+H]⁺.

### Example 65: (S)-4-methyl-N-(3-(1-((4-methyl-4H-1,2,4-triazol-3-yl)thio)ethyl)phenyl)-6-(trifluoromethyl)picolinamide (65)

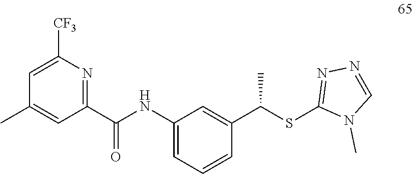 [SMILES: Cc1cc(C(=O)Nc2cccc([C@H](C)Sc3nncn3C)c2)nc(C(F)(F)F)c1]

Step 1: Methyl 4-methyl-6-(trifluoromethyl)picolinate: A suspension of potassium carbonate (0.878 g, 6.26 mmol), trimethylboroxine (3.5 M in tetrahydrofuran, 1.19 mL, 4.17 mmol), 1,1′-bis(diphenylphosphino)ferrocene-palladium(II)dichloride dichloromethane complex (0.177 g, 0.209 mmol) and methyl 4-chloro-6-(trifluoromethyl)pyridine-2-carboxylate (0.500 g, 2.09 mmol) in dioxane-water (10:1 v/v, 1.7 mL) was purged with nitrogen and heated to 80° C. under nitrogen for 1 h. The reaction mixture was diluted with water and ethyl acetate. General Work-up Procedure 1 was followed. Flash column chromatography afforded the title compound as an off-white powder. Yield: 0.229 g (50.0%).

Step 2: synthesis of 65: Followed Example 63, step 3 to afford 65 (0.121 g, 62.9%). ¹H NMR (500 MHz, DMSO-d6) δ 10.38 (s, 1H), 8.55 (s, 1H), 8.24 (s, 1H), 8.08-8.03 (m, 1H), 7.86 (t, J=1.9 Hz, 1H), 7.81 (ddd, J=8.2, 2.2, 1.0 Hz, 1H), 7.32 (t, J=7.9 Hz, 1H), 7.07 (dt, J=7.7, 1.3 Hz, 1H), 4.69 (q, J=6.9 Hz, 1H), 3.40 (s, 3H), 2.57 (s, 3H), 1.67 (d, J=6.9 Hz, 3H). LCMS: C₁₉H₁₈F₃N₅OS requires: 421, found: m/z=422 [M+H]⁺.

### Example 66: (S)-6-chloro-N-(3-(1-((4-methyl-4H-1,2,4-triazol-3-yl)thio)ethyl)phenyl)-4-(trifluoromethyl)picolinamide (66)

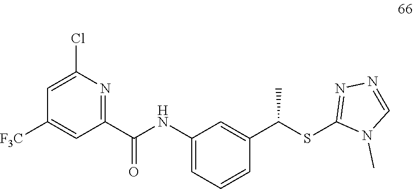 [SMILES: C[C@H](Sc1nncn1C)c1cccc(NC(=O)c2cc(C(F)(F)F)cc(Cl)n2)c1]

Followed Example 57a to afford 66 (0.489 g, 86.5%) as an off-white solid. ¹H NMR (500 MHz, Chloroform-d) δ 9.59 (s, 1H), 8.48 (s, 1H), 8.10 (s, 1H), 7.79 (s, 1H), 7.75-7.68 (m, 2H), 7.38-7.30 (m, 1H), 7.10 (d, J=8.4 Hz, 1H), 4.84 (d, J=7.3 Hz, 1H), 3.35 (s, 3H), 1.85 (d, J=1.2 Hz, 3H). LCMS: C₁₈H₁₅ClF₃N₅OS requires: 441, found: m/z=442 [M+H]⁺.

### Example 67: (S)-4-cyclopropyl-N-(3-(1-((4-methyl-4H-1,2,4-triazol-3-yl)thio)ethyl)phenyl)-6-(trifluoromethyl)picolinamide (67)

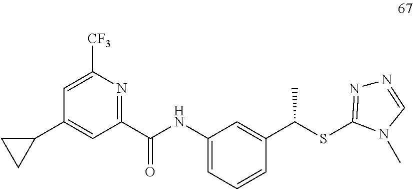 [SMILES: C[C@H](Sc1nncn1C)c1cccc(NC(=O)c2cc(C3CC3)cc(C(F)(F)F)n2)c1]

Step 1: Methyl 4-cyclopropyl-6-(trifluoromethyl)picolinate: A suspension of potassium carbonate (0.351 g, 2.50 mmol), cyclopropylboronic acid pinacol ester (0.61 mL, 3.3 mmol), 1,1′-bis(diphenylphosphino)ferrocene-palladium(II)dichloride dichloromethane complex (0.0706 g, 0.0835 mmol) and methyl 4-chloro-6-(trifluoromethyl)pyridine-2-carboxylate (0.200 g, 0.835 mmol) in dioxane-water (10:1 v/v, 1.0 mL) was purged with nitrogen and heated to 80° C. under nitrogen for 4 h. The mixture was diluted with water and EtOAc. General Work-up Procedure 1 was followed. Flash column chromatography afforded the title compound as an off-white powder. Yield: 0.106 g (51.8%).

Step 2: synthesis of 67: Followed Example 63, step 3 to afford 67 (0.0739 g, 81.0%). ¹H NMR (500 MHz, DMSO-d6) δ 10.36 (s, 1H), 8.56 (s, 1H), 8.05 (d, J=1.6 Hz, 1H), 7.87 (dd, J=6.9, 1.8 Hz, 2H), 7.83-7.77 (m, 1H), 7.32 (t, J=7.9 Hz, 1H), 7.06 (dt, J=7.7, 1.3 Hz, 1H), 4.69 (q, J=7.0 Hz, 1H), 3.39 (s, 3H), 2.30 (tt, J=8.3, 4.9 Hz, 1H), 1.67 (d, J=7.0 Hz, 3H), 1.27-1.16 (m, 2H), 1.05 (dt, J=7.1, 4.6 Hz, 2H). LCMS: C₂₁H₂₀F₃N₅OS requires: 447, found: m/z=448 [M+H]⁺.

### Example 68: (S)-4-chloro-N-(3-(1-((4-methyl-4H-1,2,4-triazol-3-yl)thio)ethyl)phenyl)-6-(trifluoromethyl)picolinamide (68)

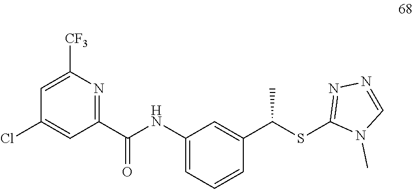 [SMILES: C[C@H](Sc1nncn1C)c1cccc(NC(=O)c2cc(Cl)cc(C(F)(F)F)n2)c1]

Followed Example 63, step 3 to afford 68. Yield: 0.497 g (82%). ¹H NMR (500 MHz, DMSO-d6) δ 10.52 (s, 1H), 8.54 (s, 1H), 8.46-8.40 (m, 2H), 7.86 (t, J=1.9 Hz, 1H), 7.80 (ddd, J=8.1, 2.2, 1.0 Hz, 1H), 7.33 (t, J=7.9 Hz, 1H), 7.08 (dt, J=7.7, 1.3 Hz, 1H), 4.70 (q, J=7.0 Hz, 1H), 3.39 (s, 3H), 1.67 (d, J=7.0 Hz, 3H). LCMS: C₁₈H₁₅ClF₃N₅OS requires: 441, found: m/z=442 [M+H]⁺.

### Example 69: (S)-6-cyclopropyl-N-(3-(1-((4-methyl-4H-1,2,4-triazol-3-yl)thio)ethyl)phenyl)-4-(trifluoromethyl)picolinamide (69)

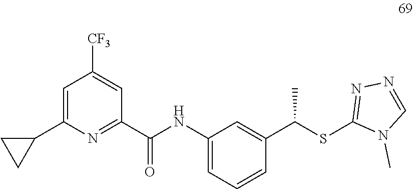 [SMILES: C[C@H](Sc1nncn1C)c1cccc(NC(=O)c2cc(C(F)(F)F)cc(C3CC3)n2)c1]

Followed Example 67, step 1 to afford the title compound as an off-white powder. Yield: 0.0770 g, (75.2%).

Step 2: synthesis of 69: Followed Example 63, step 3 to afford 69. Yield: 0.0814 g (57.9%). ¹H NMR (500 MHz, DMSO-d6) δ 10.30 (s, 1H), 8.55 (s, 1H), 8.06 (d, J=1.5 Hz, 1H), 7.99 (d, J=1.5 Hz, 1H), 7.83 (t, J=1.9 Hz, 1H), 7.76 (dd, J=8.0, 2.0 Hz, 1H), 7.34 (t, J=7.9 Hz, 1H), 7.08 (dt, J=7.7, 1.3 Hz, 1H), 4.70 (q, J=6.9 Hz, 1H), 3.40 (s, 3H), 2.09 (d, J=5.8 Hz, 1H), 1.68 (d, J=7.0 Hz, 3H), 1.32 (tt, J=5.0, 2.7 Hz, 2H), 1.19-1.10 (m, 2H). LCMS: C₂₁H₂₀F₃N₅OS requires: 447, found: m/z=448 [M+H]⁺.

### Example 70: (S)-6-cyclopropyl-5-methyl-N-(3-(1-((4-methyl-4H-1,2,4-triazol-3-yl)thio)ethyl)phenyl)pyrazine-2-carboxamide (70)

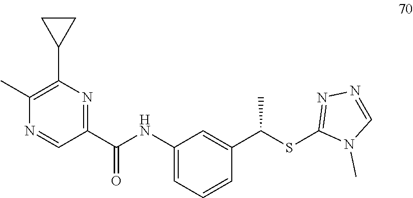 [SMILES: Cc1ncc(C(=O)Nc2cccc([C@H](C)Sc3nncn3C)c2)nc1C1CC1]

Step 1: Methyl 6-bromo-5-methylpyrazine-2-carboxylate: A solution of methyl 5-methylpyrazine-2-carboxylate (10.0 g, 65.7 mmol) in water (164 mL) at 75° C. was treated with a solution of bromine (3.70 mL, 72.3 mmol) and potassium bromide (15.6 g, 131 mmol) in minimum amount of water over 5 min. The mixture was stirred at 75° C. for 1 h. The mixture was quenched with saturated aqueous sodium thiosulfate, basified with saturated aqueous sodium bicarbonate. General Work-up Procedure 1 was followed. Flash column chromatography afforded the title compound as an off-white solid. Yield: 3.76 g (24.8%).

Step 2: Methyl 6-cyclopropyl-5-methylpyrazine-2-carboxylate: Followed Example 67, step 1 to afford the title compound as an off-white powder. Yield: 0.780 g, (74.4%).

Step 3: synthesis of 70: Followed Example 63, step 3 to afford 70. Yield: 0.0601 g (65.5%). ¹H NMR (500 MHz, DMSO-d6) δ 10.12 (s, 1H), 8.87 (s, 1H), 8.55 (s, 1H), 7.81 (t, J=1.9 Hz, 1H), 7.78-7.70 (m, 1H), 7.32 (t, J=1.9 Hz, 1H), 7.07 (dt, J=7.6, 1.3 Hz, 1H), 4.70 (q, J=7.0 Hz, 1H), 3.40 (s, 3H), 2.74 (s, 3H), 2.34 (ddd, J=8.1, 4.8, 3.3 Hz, 1H), 1.67 (d, J=7.0 Hz, 3H), 1.32 (ddd, J=6.9, 4.9, 2.6 Hz, 2H), 1.14-1.06 (m, 2H). LCMS: C₂₀H₂₂N₆OS requires: 394, found: m/z=395 [M+H]⁺.

### Example 71: (S)-4-methoxy-N-(3-(1-((4-methyl-4H-1,2,4-triazol-3-yl)thio)ethyl)phenyl)-6-(trifluoromethyl)picolinamide (71)

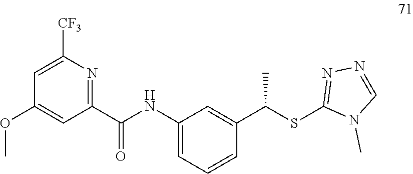 [SMILES: COc1cc(C(=O)Nc2cccc([C@H](C)Sc3nncn3C)c2)nc(C(F)(F)F)c1]

Step 1: Methyl 4-methoxy-6-(trifluoromethyl)picolinate: A solution of methyl 4-chloro-6-(trifluoromethyl)pyridine-2-carboxylate (0.100 g, 0.417 mmol) in methanol was treated with a fresh solution of sodium methoxide (25% solution in methanol, 0.19 mL, 0.83 mmol) and the solution was stirred at rt for 4 h. The reaction was quenched with acetic acid (0.048 mL, 0.83 mmol), diluted with water. General Work-up Procedure 1 was followed. Purification on silica gel (ethyl acetate in hexanes 0% to 100%) yielded desired the title compound as an off-white powder. Yield: 0.0754 g, (76.8%).

Step 2: synthesis of 71: Followed Example 63, step 3 to afford 71. Yield: 0.106 g (75.3%). ¹H NMR (500 MHz, DMSO-d6) δ 10.37 (s, 1H), 8.55 (s, 1H), 7.89-7.85 (m, 2H), 7.83-7.78 (m, 1H), 7.71 (d, J=2.4 Hz, 1H), 7.32 (t, J=7.9 Hz, 1H), 7.07 (dt, J=7.7, 1.3 Hz, 1H), 4.69 (q, J=6.9 Hz, 1H), 4.05 (s, 3H), 3.39 (s, 3H), 1.67 (d, J=6.9 Hz, 3H). LCMS: C₁₉H₁₈F₃N₅O₂S requires: 437, found: m/z=438 [M+H]⁺.

### Example 72: (S)-6-cyclopropyl-N-(3-(1-((4-methyl-4H-1,2,4-triazol-3-yl)thio)ethyl)phenyl)picolinamide (72)

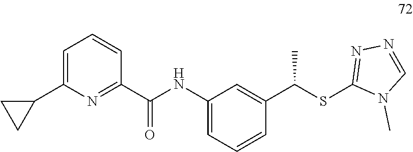 [SMILES: C[C@H](Sc1nncn1C)c1cccc(NC(=O)c2cccc(C3CC3)n2)c1]

Followed Example 62 to afford compound 72 as an off-white powder. Yield: 0.0216 g (51.2%). ¹H NMR (500 MHz, DMSO-d6) δ 10.20 (s, 1H), 8.57 (s, 1H), 7.95-7.87 (m, 2H), 7.84 (t, J=2.0 Hz, 1H), 7.79-7.72 (m, 1H), 7.53 (dd, J=7.0, 1.9 Hz, 1H), 7.32 (t, J=7.9 Hz, 1H), 7.06 (dt, J=7.7, 1.3 Hz, 1H), 4.70 (q, J=6.9 Hz, 1H), 3.40 (s, 3H), 2.27 (tt, J=8.2, 4.8 Hz, 1H), 1.68 (d, J=6.9 Hz, 3H), 1.18 (ddd, J=6.8, 4.8, 2.6 Hz, 2H), 1.11-1.02 (m, 2H). LCMS: C₂₀H₂₁N₅OS requires: 379, found: m/z=380 [M+H]⁺.

### Example 73: (S)-6-methoxy-N-(3-(1-((4-methyl-4H-1,2,4-triazol-3-yl)thio)ethyl)phenyl)-4-(trifluoromethyl)picolinamide (73)

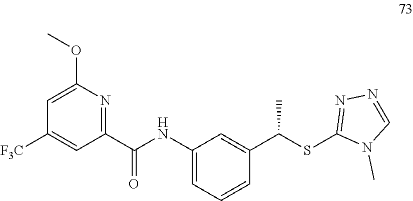 [SMILES: COc1cc(C(F)(F)F)cc(C(=O)Nc2cccc([C@H](C)Sc3nncn3C)c2)n1]

Followed Example 71, step 1 to afford compound 73 as an off-white solid. Yield: 0.0179 g (32.6%). ¹H NMR (500 MHz, DMSO-d6) δ 10.33 (s, 1H), 8.56 (s, 1H), 7.91 (d, J=1.3 Hz, 1H), 7.83 (t, J=1.9 Hz, 1H), 7.81-7.74 (m, 1H), 7.57-7.53 (m, 1H), 7.34 (t, J=7.9 Hz, 1H), 7.10 (dt, J=7.7, 1.3 Hz, 1H), 4.70 (q, J=6.9 Hz, 1H), 4.17 (s, 3H), 3.40 (s, 3H), 1.68 (d, J=7.0 Hz, 3H). LCMS: C₁₉H₁₈F₃N₅O₂S requires: 437, found: m/z=438 [M+H]⁺.

### Example 74: N-[3-[(1 S)-1-[(4-methyl-1,2,4-triazol-3-yl)sulfanyl]ethyl]phenyl]-4-(trifluoromethyl)thiazole-2-carboxamide (74)

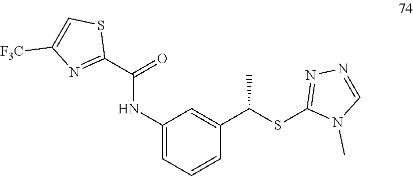 [SMILES: C[C@H](Sc1nncn1C)c1cccc(NC(=O)c2nc(C(F)(F)F)cs2)c1]

1-Propanephosphonic anhydride (0.05 mL, 0.16 mmol, 1.5 eq.) was added to a solution of 4-(trifluoromethyl)thiazole-2-carboxylic acid (32 mg, 0.16 mmol, 1.5 eq.), A-a (25 mg, 0.11 mmol, 1 eq.), and 4-methylmorpholine (0.12 mL, 1.1 mmol, 10 eq.) in N,N-dimethylformamide (0.25 mL) at rt. The solution was maintained at rt for 48 h. Compound 74 (2.0 mg) was obtained as a colorless solid using standard HPLC purification methods. ¹H NMR (500 MHz, Methanol-d₄) δ 8.56 (s, 1H), 8.52-8.48 (m, 1H), 7.74 (q, J=1.9 Hz, 1H), 7.65 (ddd, J=8.2, 2.2, 1.0 Hz, 1H), 7.31 (t, J=7.9 Hz, 1H), 7.09 (d, J=7.6 Hz, 1H), 4.70 (q, J=6.9 Hz, 1H), 3.34 (s, 3H), 1.77 (dd, J=7.0, 1.3 Hz, 3H). LCMS: C₁₆H₁₄F₃N₅OS₂ requires: 413, found: m/z=414 [M+H]⁺.

### Example 75: N-[3-[(1S)-1-[(4-methyl-1,2,4-triazol-3-yl)sulfanyl]ethyl]phenyl]-2-(trifluoromethyl)pyrimidine-4-carboxamide (75)

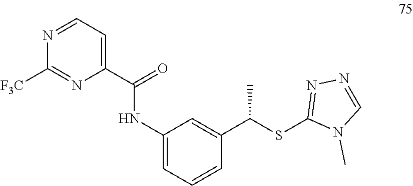 [SMILES: C[C@H](Sc1nncn1C)c1cccc(NC(=O)c2ccnc(C(F)(F)F)n2)c1]

Followed General procedure 2 with heating at 40° C. Solvent extraction was omitted; The mixture was purified by HPLC to afford the title compound as a colorless solid: ¹H NMR (500 MHz, DMSO-d6) δ 10.70 (s, 1H), 9.36 (d, J=5.0 Hz, 1H), 8.63 (s, 1H), 8.37 (d, J=5.0 Hz, 1H), 7.87 (s, 1H), 7.80 (ddd, J=8.1, 2.1, 1.0 Hz, 1H), 7.34 (t, J=7.9 Hz, 1H), 7.10 (dt, J=7.6, 1.3 Hz, 1H), 4.71 (q, J=7.0 Hz, 1H), 3.41 (s, 3H), 1.67 (d, J=7.0 Hz, 3H); LCMS: C₁₇H₁₅F₃N₆OS requires: 408, found: m/z=409 [M+H]+.

### Example 76: 2-cyclopropyl-A-[3-[(1S)-1-[(4-methyl-1,2,4-triazol-3-yl)sulfanyl]ethyl]phenyl]pyrimidine-4-carboxamide (76)

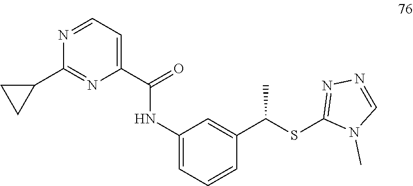 [SMILES: C[C@H](Sc1nncn1C)c1cccc(NC(=O)c2ccnc(C3CC3)n2)c1]

Followed General procedure 2. The mixture was purified by HPLC then silica gel chromatography to afford the title compound as a colorless solid: ¹H NMR (500 MHz, DMSO-d₆) δ 10.52 (s, 1H), 8.93 (d, J=5.0 Hz, 1H), 8.58 (s, 1H), 7.88 (t, J=1.9 Hz, 1H), 7.85-7.76 (m, 2H), 7.32 (t, J=7.9 Hz, 1H), 7.08 (dt, J=7.6, 1.3 Hz, 1H), 4.69 (q, J=6.9 Hz, 2H), 3.40 (s, 3H), 2.40 (tt, J=8.2, 6.3, 4.0 Hz, 1H), 1.67 (d, J=7.0 Hz, 2H), 1.23-1.11 (m, 4H); LCMS: C₁₉H₂₀N₆OS requires: 380, found: m/z=381 [M+H]⁺.

### Example 77: N-[3-[(1S)-1-[(4-methyl-1,2,4-triazol-3-yl)sulfanyl]ethyl]phenyl]-6-(trifluoromethyl)pyrazine-2-carboxamide (77)

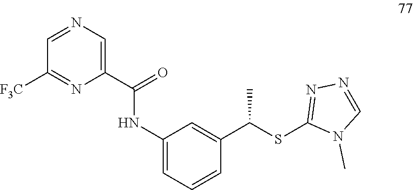 [SMILES: C[C@H](Sc1nncn1C)c1cccc(NC(=O)c2cncc(C(F)(F)F)n2)c1]

Followed General procedure 2 to afford the title compound as a colorless solid: ¹H NMR (500 MHz, DMSO-d₆) δ 10.66 (s, 1H), 9.56 (s, 1H), 9.46 (s, 1H), 8.65 (s, 1H), 7.87 (t, J=1.9 Hz, 1H), 7.80 (ddd, J=8.2, 2.2, 1.0 Hz, 1H), 7.34 (t, J=7.9 Hz, 1H), 7.09 (dt, J=7.7, 1.4 Hz, 1H), 4.71 (q, J=7.0 Hz, 1H), 3.41 (s, 3H), 1.68 (d, J=7.0 Hz, 3H). LCMS: C₁₇H₁₅F₃N₆OS requires: 408, found: m/z=409 [M+H]⁺.

### Example 78: 4-chloro-A-[3-[(LS)-1-[(4-methyl-1,2,4-triazol-3-yl)sulfanyl]ethyl]phenyl]-1H-indole-2-carboxamide (78)

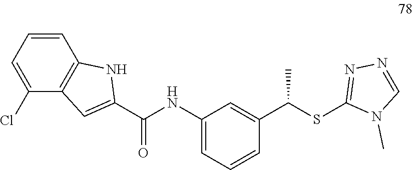 [SMILES: C[C@H](Sc1nncn1C)c1cccc(NC(=O)c2cc3c(Cl)cccc3[nH]2)c1]

Step 1: Synthesis of 4-chloro-1H-indole-2-carbonyl fluoride: To a suspension of 4-chloro-1H-indole-2-carboxylic acid (0.15 g, 0.77 mmol, 1 eq.), cyanuric fluoride (0.070 mL, 0.81 mmol, 1.1 eq.), and dichloromethane (1.0 mL) was added pyridine (0.06 mL, 0.81 mmol, 1.1 eq.) in one portion at rt and maintained for 1 h. The mixture was poured into ice water (10 mL) and dichloromethane (10 mL), followed by filtration through a polypropylene frit. The phases were separated and the aqueous layer extracted with dichloromethane (2×5 mL). The combined organic phases were washed with brine (1×5 mL), dried, filtered and concentrated to a tan solid, which was used without purification.

Step 2: Synthesis of 78: To a solution of A-a (0.03 g, 0.12 mmol, 1 eq.), 4-chloro-1H-indole-2-carbonyl fluoride (0.03 g, 0.13 mmol, 1.1 eq.), and N,N-dimethylformamide (0.3 mL) was added pyridine (0.05 mL, 0.58 mmol, 5 eq.) in one portion. The solution was maintained at rt for 24 h, then purified by HPLC to afford the title compound (44 mg) as a colorless solid: ¹H NMR (500 MHz, DMSO-d₆) δ 12.12 (d, J=2.4 Hz, 1H), 10.36 (s, 1H), 8.62 (s, 1H), 7.84-7.76 (m, 2H), 7.56 (dd, J=2.3, 0.9 Hz, 1H), 7.45 (dt, J=8.1, 0.9 Hz, 1H), 7.31 (t, J=7.9 Hz, 1H), 7.26-7.19 (m, 1H), 7.17 (dd, J=7.5, 0.9 Hz, 1H), 7.04 (dt, J=7.6, 1.4 Hz, 1H), 4.69 (q, J=6.9 Hz, 1H), 3.41 (s, 3H), 1.68 (d, J=7.0 Hz, 3H); LCMS: C₂₀H₁₈ClN₅OS requires: 411, found: m/z=412 [M+H]⁺.

### Example 79: (S)-N-(3-(1-((4-methyl-4H-1,2,4-triazol-3-yl)thio)ethyl)phenyl)-3-(trifluoromethoxy)benzamide (79)

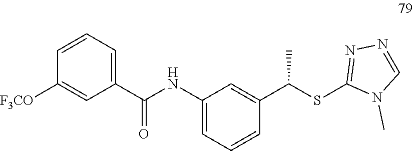 [SMILES: C[C@H](Sc1nncn1C)c1cccc(NC(=O)c2cccc(OC(F)(F)F)c2)c1]

Followed Example 56 to give compound 79 (28 mg, 73% yield). ¹H NMR (500 MHz, DMSO-d₆) δ 10.39 (s, 1H), 8.53 (s, 1H), 8.01 (dt, J=7.7, 1.3 Hz, 1H), 7.91 (s, 1H), 7.75 (t, J=1.9 Hz, 1H), 7.73-7.65 (m, 2H), 7.65-7.56 (m, 1H), 7.29 (t, J=7.9 Hz, 1H), 7.08-6.97 (m, 1H), 4.66 (q, J=6.9 Hz, 1H), 3.38 (s, 2H), 1.64 (d, J=7.0 Hz, 2H). LCMS: C₁₉H₁₇F₃N₄O₂S requires: 422.4, found: m/z 423.4 [M+H]⁺.

### Example 80: (S)-N-(3-(1-((4-methyl-4H-1,2,4-triazol-3-yl)thio)ethyl)phenyl)-6-(methylsulfonyl)picolinamide (80)

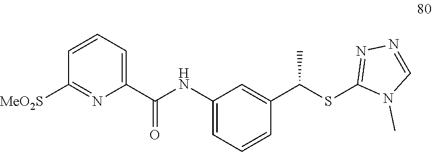 [SMILES: C[C@H](Sc1nncn1C)c1cccc(NC(=O)c2cccc(S(C)(=O)=O)n2)c1]

Followed Example 56 to give compound 80 (19 mg, 46% yield). ¹H NMR (500 MHz, DMSO-d₆) δ 10.52 (s, 1H), 8.53 (s, 1H), 8.44-8.34 (m, 2H), 8.27 (dd, J=7.1, 1.7 Hz, 1H), 7.84 (t, J=2.0 Hz, 1H), 7.81-7.74 (m, 1H), 7.33 (t, J=7.9 Hz, 1H), 7.10-7.05 (m, 1H), 4.69 (q, J=6.9 Hz, 1H), 3.59 (s, 3H), 3.39 (s, 3H), 1.66 (d, J=7.0 Hz, 3H). LCMS: C₁₈H₁₉N₅O₃S₂ requires: 417.5, found: m/z 418.4 [M+H]⁺.

### Example 81: (S)-6-(1,1-difluoroethyl)-N-(3-(1-((4-methyl-4H-1,2,4-triazol-3-yl)thio)ethyl)phenyl)picolinamide (81)

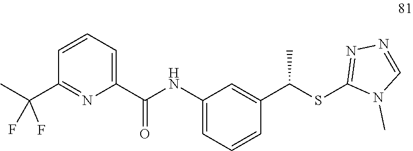 [SMILES: C[C@H](Sc1nncn1C)c1cccc(NC(=O)c2cccc(C(C)(F)F)n2)c1]

Followed Example 108 using A-a to afford compound 81 (30 mg, 77% yield). ¹H NMR (500 MHz, DMSO-d₆) δ 10.37 (s, 1H), 8.56 (s, 1H), 8.29-8.16 (m, 2H), 7.97 (dd, J=6.8, 2.1 Hz, 1H), 7.85 (t, J=2.0 Hz, 1H), 7.78 (ddd, J=8.1, 2.2, 1.0 Hz, 1H), 7.32 (t, J=7.9 Hz, 1H), 7.07 (dt, J=7.6, 1.3 Hz, 1H), 4.69 (q, J=6.9 Hz, 1H), 3.39 (s, 3H), 2.21 (t, J=19.5 Hz, 3H), 1.67 (d, J=7.0 Hz, 3H). LCMS: C₁₉H₁₉F₂N₅OS requires: 403.5, found: m/z 404.4 [M+H]⁺.

### Example 82: (S)-6-methyl-N-(3-(1-((4-methyl-4H-1,2,4-triazol-3-yl)thio)ethyl)phenyl)pyrazine-2-carboxamide (82)

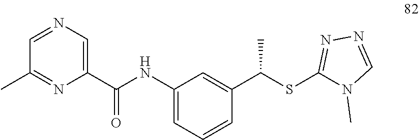 [SMILES: Cc1cncc(C(=O)Nc2cccc([C@H](C)Sc3nncn3C)c2)n1]

Followed Example 108 using A-a to afford compound 82. Yield: 42 mg (83%). ¹H NMR (500 MHz, DMSO-d₆) δ 10.52 (s, 1H), 9.07 (s, 1H), 8.81 (s, 1H), 8.57 (s, 1H), 7.90 (t, J=1.9 Hz, 1H), 7.81 (ddd, J=8.2, 2.2, 1.0 Hz, 1H), 7.30 (t, J=7.9 Hz, 1H), 7.04 (dt, J=7.8, 1.3 Hz, 1H), 4.67 (q, J=6.9 Hz, 1H), 3.38 (s, 3H), 2.66 (s, 3H), 1.66 (d, J=6.9 Hz, 3H). LCMS: C₁₇H₁₈N₆OS requires: 354.4, found: m/z 355.4 [M+H]⁺.

### Example 83: (S)-N-(3-(1-((4-methyl-4H-1,2,4-triazol-3-yl)thio)ethyl)phenyl)-4-(trifluoromethyl)quinoline-2-carboxamide (83)

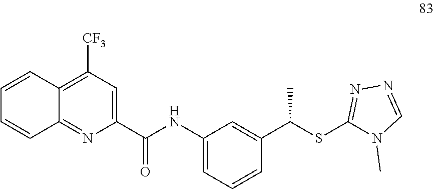 [SMILES: C[C@H](Sc1nncn1C)c1cccc(NC(=O)c2cc(C(F)(F)F)c3ccccc3n2)c1]

Followed Example 108 using A-a to afford compound 83 (33 mg, 85%). ¹H NMR (500 MHz, DMSO-d₆) δ 10.89 (s, 1H), 8.57 (s, 1H), 8.50 (s, 1H), 8.46 (dd, J=8.6, 1.2 Hz, 1H), 8.22 (d, J=8.6 Hz, 1H), 8.10 (ddd, J=8.3, 6.9, 1.3 Hz, 1H), 8.04-7.96 (m, 2H), 7.94-7.88 (m, 1H), 7.34 (t, J=7.9 Hz, 1H), 7.08 (dt, J=7.8, 1.3 Hz, 1H), 4.70 (q, J=6.9 Hz, 1H), 3.40 (s, 3H), 1.68 (d, J=6.9 Hz, 3H). LCMS: C₂₂H₁₈F₃N₅OS requires: 457.5, found: m/z 458.4 [M+H]⁺.

### Example 84: (S)-4-(3-hydroxyoxetan-3-yl)-N-(3-(1-((4-methyl-4H-1,2,4-triazol-3-yl)thio)ethyl)phenyl)-6-(trifluoromethyl)picolinamide (84)

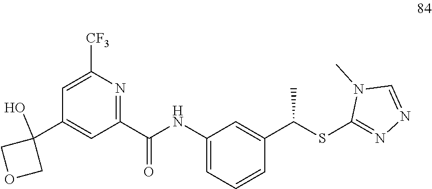 [SMILES: C[C@H](Sc1nncn1C)c1cccc(NC(=O)c2cc(C3(O)COC3)cc(C(F)(F)F)n2)c1]

Step 1: 4-(3-hydroxyoxetan-3-yl)-6-(trifluoromethyl)pyridine-2-carboxylic acid. Under an atmosphere of argon at −78° C., n-BuLi (2.5 M hexanes, 0.33 mL, 0.81 mmol, 2.2 eq.) was added to a THF (3 mL) solution containing 4-bromo-6-(trifluoromethyl)pyridine-2-carboxylic acid (0.1 g, 0.3700 mmol, 1 eq.). After 5 min., oxetan-3-one (0.06 mL, 0.81 mmol, 2.2 eq.) was added to the solution and the mixture was stirred at −78° C. for 1 h. The solution was then quenched with a 1 N hydrochloric acid solution and partitioned with EtOAc, dried, and concentrated to dryness. This material was used without purification.

Step 2: synthesis of 84: Followed General procedure 2 using trimethylamine instead of N-ethyl-N-isopropylpropan-2-amine to afford 84 (4.5 mg, 2.5%). ¹H NMR (500 MHz, Chloroform-d) δ 9.71 (s, 1H), 8.81 (s, 2H), 8.26 (d, J=1.5 Hz, 1H), 7.58 (t, J=1.9 Hz, 1H), 7.45 (dd, J=8.1, 1.8 Hz, 1H), 7.36 (t, J=7.8 Hz, 1H), 7.16 (d, J=7.6 Hz, 1H), 5.05 (d, J=7.2 Hz, 2H), 4.86-4.78 (m, 3H), 3.41 (s, 3H), 1.86 (d, J=7.1 Hz, 3H). LCMS: C₂₁H₂₀F₃N₅O₃S requires: 479.1, found: m/z=480.4 [M+H]⁺.

### Example 85: 4-(1-hydroxy-1-methyl-ethyl)-N-[3-[(1S)-1-[(4-methyl-1,2,4-triazol-3-yl)sulfanyl]ethyl]phenyl]-6-(trifluoromethyl)pyridine-2-carboxamide (85)

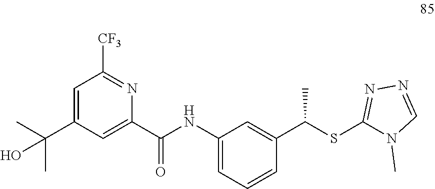 [SMILES: C[C@H](Sc1nncn1C)c1cccc(NC(=O)c2cc(C(C)(C)O)cc(C(F)(F)F)n2)c1]

Step 1: 4-(1-hydroxy-1-methyl-ethyl)-6-(trifluoromethyl)pyridine-2-carboxylic acid. Under an atmosphere of argon at −78° C., n-BuLi (2.5 M in hexanes, 0.33 mL, 0.81 mmol, 2.2 eq.) was added to a THF (3 mL) solution containing 4-bromo-6-(trifluoromethyl)pyridine-2-carboxylic acid (0.1 g, 0.37 mmol, 1 eq.). After 5 min. acetone (0.11 mL, 1.48 mmol, 4 eq.) was added to the solution and the mixture was stirred at −78° C. for 1 h. The solution was then quenched with a 1 N hydrochloric acid solution and partitioned with EtOAc, dried, and concentrated to dryness. This material was used without purification.

Step 2: synthesis of 85: Followed Example 84, step 2 to afford compound 85 (7.1 mg, yield 3.5%). ¹H NMR (500 MHz, Chloroform-d) δ 9.77 (s, 1H), 8.51 (d, J=9.2 Hz, 2H), 8.07 (d, J=1.4 Hz, 1H), 7.76 (t, J=1.9 Hz, 1H), 7.54 (dd, J=8.1, 1.7 Hz, 1H), 7.32 (t, J=7.8 Hz, 1H), 7.07 (d, J=7.6 Hz, 1H), 4.83 (q, J=7.0 Hz, 1H), 3.38 (s, 3H), 2.01 (s, 1H), 1.84 (d, J=7.0 Hz, 3H), 1.66 (s, 6H). LCMS: C₂₁H₂₂F₃N₅O₂S requires: 465.1, found: m/z=466.4 [M+H]⁺.

### Example 86: 4-(2-hydroxyethylsulfonylamino)-N-[3-[(1S)-1-[(4-methyl-1,2,4-triazol-3-yl)sulfanyl]ethyl]phenyl]pyridine-2-carboxamide (86)

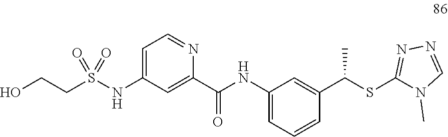 [SMILES: C[C@H](Sc1nncn1C)c1cccc(NC(=O)c2cc(NS(=O)(=O)CCO)ccn2)c1]

Compound 86 (5 mg, 29%) was obtained according to procedures disclosed herein. ¹H NMR (500 MHz, DMSO-d6) δ 10.76 (s, 1H), 10.54 (s, 1H), 8.51 (s, 1H), 8.48 (d, J=5.5 Hz, 1H), 7.90-7.84 (m, 2H), 7.73 (dd, J=7.6, 1.9 Hz, 1H), 7.31 (dd, J=5.5, 2.3 Hz, 1H), 7.22 (t, J=7.9 Hz, 1H), 6.96 (d, J=7.7 Hz, 1H), 4.60 (q, J=7.0 Hz, 2H), 3.72 (t, J=6.2 Hz, 2H), 3.39 (d, J=12.3 Hz, 2H), 3.32 (s, 3H), 1.59 (d, J=7.0 Hz, 3H). LCMS: C₁₉H₂₂N₆O₄S₂ requires: 462.5, found: m/z=463.4 [M+H]⁺.

### Example 87: 6-(fluoromethyl)-N-[3-[(1S)-1-[(4-methyl-1,2,4-triazol-3-yl)sulfanyl]ethyl]phenyl]pyridine-2-carboxamide (87)

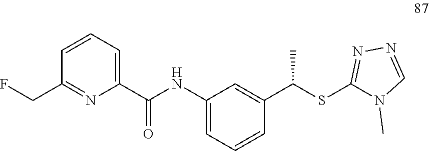 [SMILES: C[C@H](Sc1nncn1C)c1cccc(NC(=O)c2cccc(CF)n2)c1]

To a stirring solution of A-a (60 mg, 0.26 mmol, 1 eq.) and methyl 6-(fluoromethyl)pyridine-2-carboxylate (0.13 g, 0.77 mmol, 3 eq.) in tetrahydrofuran (2.5 mL) was added trimethylaluminum (0.38 mL, 0.77 mmol, 3 eq.). The mixture was allowed to stir at reflux for 2 h and then cooled to room temperature. The reaction was quenched with potassium sodium tartrate tetrahydrate and extracted with EtOAc. The combined organic layers were washed with water, dried over sodium sulfate, filtered, and concentrated under reduced pressure. The residue was purified by HPLC to afford Compound 87 (10 mg, 11% yield). ¹H NMR (500 MHz, DMSO-d6) δ 10.48 (s, 1H), 8.60 (s, 1H), 8.21-8.10 (m, 2H), 7.90 (t, J=1.9 Hz, 1H), 7.87-7.76 (m, 2H), 7.31 (t, J=7.9 Hz, 1H), 7.05 (dt, J=7.7, 1.3 Hz, 1H), 5.70 (s, 1H), 5.61 (s, 1H), 4.69 (q, J=6.9 Hz, 1H), 3.40 (s, 3H), 1.67 (d, J=6.9 Hz, 3H). LCMS: C₁₈H₁₈FN₅OS requires: 371.4, found: m/z=372.2 [M+H]⁺.

### Example 88: N-[3-[(1 S)-1-[(4-methyl-1,2,4-triazol-3-yl)sulfanyl]ethyl]phenyl]-1,3-benzothiazole-4-carboxamide (88)

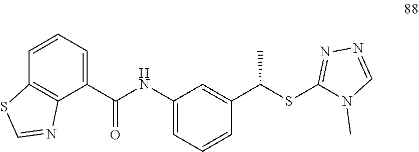 [SMILES: C[C@H](Sc1nncn1C)c1cccc(NC(=O)c2cccc3scnc23)c1]

Followed General procedure 2 to afford 88 (95 mg, 94%). ¹H NMR (500 MHz, DMSO-d6) δ 10.60 (s, 1H), 9.51 (s, 1H), 8.62 (s, 1H), 8.37 (d, J=7.5 Hz, 1H), 8.34 (dd, J=8.1, 0.9 Hz, 1H), 7.83 (t, J=1.9 Hz, 1H), 7.79-7.73 (m, 2H), 7.33 (t, J=7.9 Hz, 1H), 7.08 (dt, J=7.7, 1.3 Hz, 1H), 4.72 (q, J=6.9 Hz, 1H), 3.42 (s, 3H), 1.68 (d, J=7.0 Hz, 3H). LCMS: C₁₉H₁₇N₅OS₂ requires: 395.5, found: m/z=396.2 [M+H]⁺.

### Example 89: 6-cyclopropyl-5-(hydroxymethyl)-N-[3-[(1S)-1-[(4-methyl-1,2,4-triazol-3-yl)sulfanyl]ethyl]phenyl]pyridine-2-carboxamide (89)

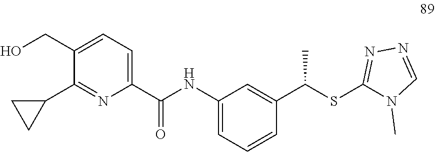 [SMILES: C[C@H](Sc1nncn1C)c1cccc(NC(=O)c2ccc(CO)c(C3CC3)n2)c1]

Step 1: Methyl 6-cyclopropyl-5-(hydroxymethyl)pyridine-2-carboxylate. To a stirring solution of methyl 6-chloro-5-formyl-pyridine-2-carboxylate (500 mg, 2.51 mmol, 1 eq.) in methanol (12 mL) was added sodium borohydride (284 mg, 7.52 mmol, 3 eq.). The mixture was stirred at room temperature for about 1 h. The reaction was diluted with 1 M hydrochloric acid followed by General Work-up Procedure 1. The crude was used in the next step.

Step 2: Methyl 6-cyclopropyl-5-(hydroxymethyl)pyridine-2-carboxylate. Followed Example 67, step 1 to yield the title compound (160 mg, 0.770 mmol, 31%) as an off-white solid.

Step 3: synthesis of 89. Followed General procedure 2 to afford compound 89 (55 mg, 52%). ¹H NMR (500 MHz, DMSO-d6) δ 10.05 (s, 1H), 8.54 (s, 1H), 7.98-7.92 (m, 1H), 7.89 (d, J=7.8 Hz, 1H), 7.80 (t, J=1.9 Hz, 1H), 7.72 (ddd, J=8.2, 2.2, 1.0 Hz, 1H), 7.31 (t, J=7.9 Hz, 1H), 7.05 (dt, J=7.6, 1.3 Hz, 1H), 5.49 (t, J=5.4 Hz, 1H), 4.78 (d, J=5.4 Hz, 2H), 4.69 (q, J=6.9 Hz, 1H), 3.39 (s, 3H), 2.22 (tt, J=8.1, 4.8 Hz, 1H), 1.67 (d, J=6.9 Hz, 3H), 1.27 (ddt, J=4.8, 3.4, 1.7 Hz, 2H), 1.01 (dp, J=6.8, 2.1, 1.7 Hz, 2H). LCMS: C₂₁H₂₃N₅O₂S requires: 409.5 found: m/z=410.2 [M+H]⁺.

### Example 90: 6-Ethoxy-N-[3-[(1S)-1-[(4-methyl-1,2,4-triazol-3-yl)sulfanyl]ethyl]phenyl]pyridine-2-carboxamide (90)

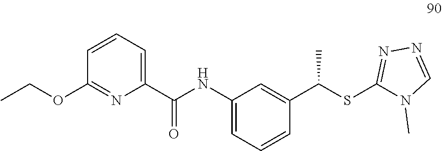 [SMILES: CCOc1cccc(C(=O)Nc2cccc([C@H](C)Sc3nncn3C)c2)n1]

Followed General procedure 2 to afford compound 90 (62 mg, 0.16 mmol, 63%). ¹H NMR (500 MHz, DMSO-d6) δ 10.14 (s, 1H), 8.54 (s, 1H), 7.94 (dd, J=8.3, 7.3 Hz, 1H), 7.83 (t, J=2.0 Hz, 1H), 7.80-7.69 (m, 2H), 7.31 (t, J=7.9 Hz, 1H), 7.06 (td, J=7.7, 7.1, 1.1 Hz, 2H), 4.69 (q, J=6.9 Hz, 1H), 4.55 (q, J=7.1 Hz, 2H), 3.39 (s, 2H), 2.48 (s, 3H), 1.67 (d, J=7.0 Hz, 2H), 1.39 (t, J=7.1 Hz, 2H). LCMS: C₁₉H₂₁N₅O₂S requires: 383.5 found: m/z=384.4 [M+H]⁺.

### Example 91: 6-Cyclopropyl-5-fluoro-N-[3-[(1S)-1-[(4-methyl-1,2,4-triazol-3-yl)sulfanyl]ethyl]phenyl]pyridine-2-carboxamide (91)

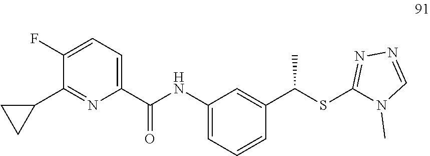 [SMILES: C[C@H](Sc1nncn1C)c1cccc(NC(=O)c2ccc(F)c(C3CC3)n2)c1]

Step 1: methyl 6-cyclopropyl-5-fluoro-pyridine-2-carboxylate. Followed Example 67, step 1 to yield the title compound (250 mg, 1.28 mmol, 78% yield) as a colorless solid. Step 2: 6-cyclopropyl-5-fluoro-N-[3-[(1S)-1-[(4-methyl-1,2,4-triazol-3-yl)sulfanyl]ethyl]phenyl]pyridine-2-carboxamide

Followed General procedure 2 to compound 91 (70 mg, 69%). ¹H NMR (500 MHz, DMSO-d6) δ 10.06 (s, 1H), 8.54 (s, 1H), 7.94 (dd, J=8.5, 4.0 Hz, 1H), 7.91-7.77 (m, 2H), 7.71 (ddd, J=8.1, 2.2, 1.0 Hz, 1H), 7.31 (t, J=7.9 Hz, 1H), 7.05 (dt, J=7.7, 1.3 Hz, 1H), 4.69 (q, J=7.0 Hz, 1H), 3.39 (s, 3H), 2.43-2.33 (m, 1H), 1.67 (d, J=7.0 Hz, 3H), 1.34 (ddt, J=5.0, 3.4, 1.8 Hz, 2H), 1.16-1.04 (m, 2H). LCMS: C₂₀H₂₀FN₅OS requires: 397.5 found: m/z=398.2 [M+H]⁺.

### Example 92: 5-cyano-6-cyclopropyl-N-[3-[(1S)-1-[(4-methyl-1,2,4-triazol-3-yl)sulfanyl]ethyl]phenyl]pyridine-2-carboxamide (92)

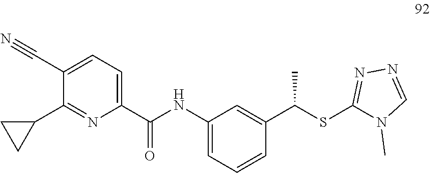 [SMILES: C[C@H](Sc1nncn1C)c1cccc(NC(=O)c2ccc(C#N)c(C3CC3)n2)c1]

Step 1: Methyl 5-cyano-6-cyclopropyl-pyridine-2-carboxylate. Followed Example 67, step 1 to yield the title compound (0.26 g, 1.29 mmol, 81% yield) as a colorless solid.

Step 2: synthesis of 92. Followed General procedure 2 to afford compound 92 (78 mg, 75% yield). ¹H NMR (500 MHz, DMSO-d6) δ 10.21 (s, 1H), 8.54 (s, 1H), 8.45 (d, J=8.0 Hz, 1H), 7.98 (d, J=8.0 Hz, 1H), 7.79 (t, J=2.0 Hz, 1H), 7.74-7.68 (m, 1H), 7.33 (t, J=7.9 Hz, 1H), 7.08 (dt, J=7.7, 1.3 Hz, 1H), 4.70 (q, J=6.9 Hz, 1H), 3.39 (s, 3H), 2.48 (m, 1H), 1.66 (d, J=7.0 Hz, 3H), 1.46 (ddt, J=4.7, 3.3, 1.9 Hz, 2H), 1.22 (dt, J=7.3, 3.7 Hz, 2H). LCMS: C₂₁H₂₀N₆OS requires: 404.5 found: m/z=405.4 [M+H]⁺.

### Example 93: 6-cyclopropyl-5-methoxy-N-[3-[(1S)-1-[(4-methyl-1,2,4-triazol-3-yl)sulfanyl]ethyl]phenyl]pyridine-2-carboxamide (93)

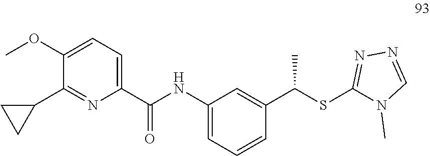 [SMILES: COc1ccc(C(=O)Nc2cccc([C@H](C)Sc3nncn3C)c2)nc1C1CC1]

Step 1: Methyl 6-cyclopropyl-5-methoxy-pyridine-2-carboxylate. Followed Example 67, step 1 to yield the title compound (120 mg, 0.58 mmol, 65% yield) as a colorless solid.

Step 2: synthesis of 93. Followed General procedure 2 to afford compound 93 (66 mg, 0.16 mmol, 63%). ¹H NMR (500 MHz, DMSO-d6) δ 9.93 (s, 1H), 8.54 (s, 1H), 7.90 (d, J=8.4 Hz, 1H), 7.79 (t, J=1.9 Hz, 1H), 7.71 (ddd, J=8.2, 2.2, 1.0 Hz, 1H), 7.51 (d, J=8.6 Hz, 1H), 7.29 (t, J=7.9 Hz, 1H), 7.03 (dt, J=7.7, 1.4 Hz, 1H), 4.68 (q, J=7.0 Hz, 1H), 3.94 (s, 3H), 3.39 (s, 3H), 1.66 (d, J=6.9 Hz, 3H), 1.23 (tt, J=4.8, 1.6 Hz, 2H), 1.00 (dq, J=8.2, 3.6 Hz, 2H). LCMS: C₂₁H₂₃N₅O₂S requires: 409.5 found: m/z=410.4 [M+H]⁺.

### Example 94: N-(5-(1-((4-methyl-4H-1,2,4-triazol-3-yl)thio)ethyl)-2-(trifluoromethoxy)phenyl)-6-(trifluoromethyl)picolinamide (94)

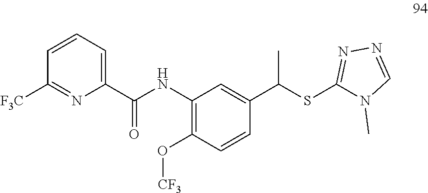 [SMILES: CC(Sc1nncn1C)c1ccc(OC(F)(F)F)c(NC(=O)c2cccc(C(F)(F)F)n2)c1]

Compound 94 was obtained (6.2 mg, 39%) according to procedures disclosed herein. ¹H NMR (500 MHz, DMSO-d₆) δ 10.39 (s, 1H), 8.54 (s, 1H), 8.49-8.38 (m, 2H), 8.36 (d, J=2.2 Hz, 1H), 8.25 (dd, J=7.3, 1.5 Hz, 1H), 7.45 (dq, J=8.5, 1.5 Hz, 1H), 7.22 (dd, J=8.6, 2.3 Hz, 1H), 4.76 (q, J=6.9 Hz, 1H), 3.39 (s, 3H), 1.67 (d, J=7.0 Hz, 3H). LCMS: C₁₉H₁₅F₆N₅O₂S requires: 491.4, found: m/z 492.4 [M+H]⁺.

### Example 95: N-(2-fluoro-5-(1-((4-methyl-4H-1,2,4-triazol-3-yl)thio)ethyl)phenyl)-6-(trifluoromethyl)picolinamide (95)

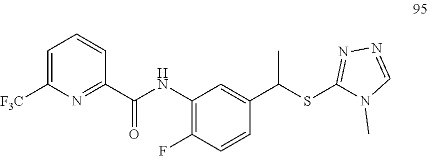 [SMILES: CC(Sc1nncn1C)c1ccc(F)c(NC(=O)c2cccc(C(F)(F)F)n2)c1]

Compound 95 was obtained according to procedures disclosed herein in 36% yield. ¹H NMR (500 MHz, Methanol-d4) δ 8.66 (s, 1H), 8.48 (d, J=7.9 Hz, 1H), 8.32 (td, J=7.9, 0.7 Hz, 1H), 8.26 (dd, J=13, 2.3 Hz, 1H), 8.08 (dd, J=7.9, 1.0 Hz, 1H), 7.18 (dd, J=10.4, 8.5 Hz, 1H), 7.12 (ddd, J=8.6, 4.9, 2.3 Hz, 1H), 4.75 (q, J=7.0 Hz, 1H), 3.50 (s, 3H), 1.77 (d, J=7.0 Hz, 3H). LCMS: C₁₈H₁₅F₄N₅OS requires: 425.1, found: m/z=426.2 [M+H]⁺.

### (S)-N-(2-fluoro-5-(1-((4-methyl-4H-1,2,4-triazol-3-yl)thio)ethyl)phenyl)-6-(trifluoromethyl)picolinamide (95a) and (R)-N-(2-fluoro-5-(1-((4-methyl-4H-1,2,4-triazol-3-yl)thio)ethyl)phenyl)-6-(trifluoromethyl)picolinamide (95b)

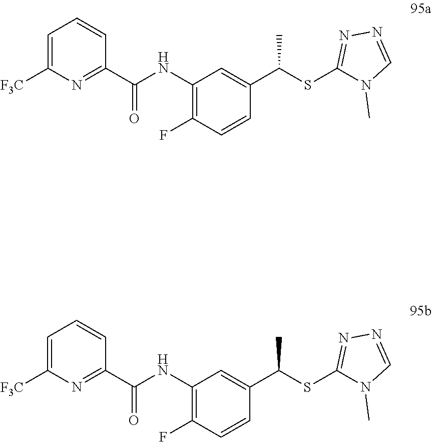 [SMILES: C[C@H](Sc1nncn1C)c1ccc(F)c(NC(=O)c2cccc(C(F)(F)F)n2)c1 ; C[C@@H](Sc1nncn1C)c1ccc(F)c(NC(=O)c2cccc(C(F)(F)F)n2)c1]

Chiral resolution of 95 using chiral prep SFC. Fractions were combined and evaporated to dryness. Lyophilization in acetonitrile/water gave the title compounds as an off-white powder.

95a (shorter retention time) LCMS: C₁₈H₁₅F₄N₅OS requires: 425, found: m/z=426 [M+H]⁺. 95b (longer retention time) LCMS: C₁₈H₁₅F₄N₅OS requires: 425, found: m/z=426 [M+H]⁺.

### Example 96: N-[2-fluoro-5-[1-[(4-methyl-1,2,4-triazol-3-yl)sulfanyl]ethyl]phenyl]quinoline-2-carboxamide (96)

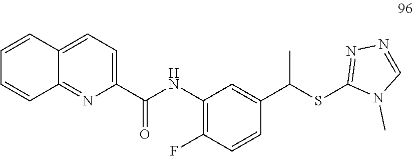 [SMILES: CC(Sc1nncn1C)c1ccc(F)c(NC(=O)c2ccc3ccccc3n2)c1]

Compound 96 was obtained according to procedures disclosed herein (35% yield). ¹H NMR (500 MHz, Methanol-d4) δ 8.64 (s, 1H), 8.55 (dd, J=8.6, 0.8 Hz, 1H), 8.34 (dd, J=7.4, 2.3 Hz, 1H), 8.31 (d, J=8.5 Hz, 1H), 8.21 (dd, J=8.5, 1.0 Hz, 1H), 8.04 (dd, J=8.3, 1.4 Hz, 1H), 7.87 (ddd, J=8.4, 6.9, 1.4 Hz, 1H), 7.73 (ddd, J=8.1, 6.9, 1.2 Hz, 1H), 7.19 (dd, 0.7=10.6, 8.5 Hz, 1H), 7.11 (ddd, J=8.5, 4.8, 2.4 Hz, 1H), 4.76 (q, J=7.0 Hz, 1H), 3.52 (s, 3H), 1.78 (d, J=7.1 Hz, 3H). LCMS: C₂₁H₁₈FN₅OS requires: 407.1, found: m/z=408 [M+H]⁺.

### (S)-N-(2-fluoro-5-(1-((4-methyl-4H-1,2,4-triazol-3-yl)thio)ethyl)phenyl)quinoline-2-carboxamide (96a) and (R)-N-(2-fluoro-5-(1-((4-methyl-4H-1,2,4-triazol-3-yl)thio)ethyl)phenyl)quinoline-2-carboxamide (96b)

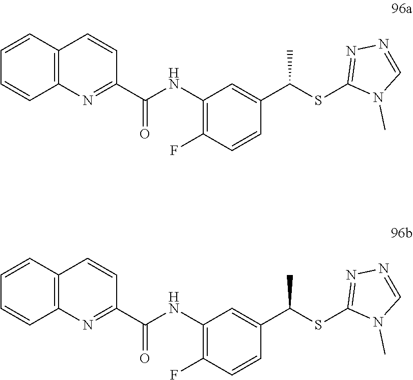 [SMILES: C[C@H](Sc1nncn1C)c1ccc(F)c(NC(=O)c2ccc3ccccc3n2)c1 ; C[C@@H](Sc1nncn1C)c1ccc(F)c(NC(=O)c2ccc3ccccc3n2)c1]

Chiral resolution of 96 using chiral prep SFC. 96a (shorter retention time) LCMS: C₂₁H₁₈FN₅OS requires: 407, found: m/z=408 [M+H]⁺. 96b (longer retention time) LCMS: C₂₁H₁₈FN₅OS requires: 407, found: m/z=408 [M+H]⁺.

### Example 97: N-(2-methoxy-5-(1-((4-methyl-4H-1,2,4-triazol-3-yl)thio)ethyl)phenyl)quinoline-2-carboxamide (97)

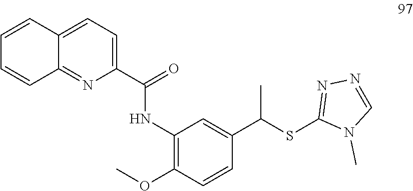 [SMILES: COc1ccc(C(C)Sc2nncn2C)cc1NC(=O)c1ccc2ccccc2n1]

Compound 97 (148 mg, 23%) was obtained according to procedures disclosed herein as an off-white solid. MS (ESI) calculated for (C₂₂H₂₁N₅O₂S) [M+H]⁺, 420.1; found, 420.2. ¹H NMR (400 MHz, DMSO-d₆) δ 10.72 (s, 1H), 8.76-8.66 (m, 1H), 8.54-8.53 (m, 2H), 8.31 (d, J=8.4 Hz, 1H), 8.22-8.20 (m, 1H), 8.15 (d, J=8.0 Hz, 1H), 7.96-7.94 (m, 1H), 7.79-7.77 (m, 1H), 7.13-7.02 (m, 2H), 4.71 (q, J=6.8 Hz, 1H), 4.00 (s, 3H), 3.43 (s, 3H), 1.67 (d, J=7.2 Hz, 3H).

### Example 98: (S)-N-(3-(1-((4H-1,2,4-triazol-3-yl)thio)ethyl)phenyl)isoquinoline-3-carboxamide (98)

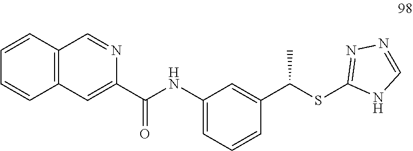 [SMILES: C[C@H](Sc1nnc[nH]1)c1cccc(NC(=O)c2cc3ccccc3cn2)c1]

Step 1: Synthesis of (R)-1-(3-aminophenyl)ethan-1-ol. To a solution of (R)-1-(3-nitrophenyl)ethanol (7.0 g, 41.9 mmol) in ethanol (120 mL) as added Fe powder (7.0 g, 126.0 mmol), ammonium chloride (13.3 g, 251.0 mmol) and water (20 mL). The mixture was stirred at 80° C. for 16 h. The mixture was filtered through a pad of Celite. The filtrate was concentrated under vacuum to give a crude solid which was dissolved in ethyl acetate (100 mL) and methanol (10 mL) and stirred for 10 min, and then filtered. The filtrate was concentrated to give (R)-1-(3-aminophenyl)ethan-1-ol as a yellow oil (6.1 g, crude), which was used for next step without further purification.

Step 2: Synthesis of (R)-N-(3-(1-hydroxyethyl)phenyl)isoquinoline-3-carboxamide. Using (R)-1-(3-aminophenyl)ethan-1-ol (6.1 g, 40.0 mmol) and isoquinoline-3-carboxylic acid, followed General Procedure 1-G with stirring at 15° C. for 1.5 h to give the title compound (7.5 g, 58%) as an off-white solid.

Step 3 (General procedure 4): Synthesis of (S)-N-(3-(1-((4H-1,2,4-triazol-3-yl)thio)ethyl)phenyl)isoquinoline-3-carboxamide. To a stirred solution of (R)—S-CA 1-hydroxyethyl)phenyl)isoquinoline-3-carboxamide (300 mg, 1.00 mmol), 4H-1,2,4-triazole-3-thiol (124 mg, 1.50 mmol) and triphenylphosphine (404 mg, 1.50 mmol) in THF (6 mL) at 0° C. was added diisopropyl azodicarboxylate (311 mg, 2.30 mmol) at 0° C. The solution was stirred at about 20° C. for 2 h. The reaction was quenched by the addition of 20 mL water. General Work-up Procedure 1 was followed to give the residue. The title compound (100 mg, 26%) was obtained as an off-white solid using standard flash chromatography purification methods. MS (ESI) calculated for (C₂₀H₁₇N₅OS) [M+H]⁺, 376.1; found, 376.2. ¹H NMR (400 MHz, DMSO-d₆) δ 14.08 (s, 1H), 10.77 (s, 1H), 9.49 (s, 1H), 8.71 (s, 1H), 8.44 (s, 1H), 8.31-8.29 (m, 2H), 8.08 (t, J=2.0 Hz, 1H), 7.98-7.83 (m, 3H), 7.33 (t, J=8.0 Hz, 1H), 7.18-7.17 (m, 1H), 4.84 (q, J=6.8 Hz, 1H), 1.69 (d, J=6.8 Hz, 3H).

### Example 99: (S)-N-(3-(1-((4-cyclopropyl-4H-1,2,4-triazol-3-yl)thio)ethyl)phenyl)isoquinoline-3-carboxamide (99)

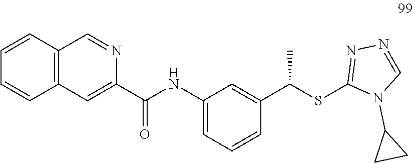 [SMILES: C[C@H](Sc1nncn1C1CC1)c1cccc(NC(=O)c2cc3ccccc3cn2)c1]

Followed General procedure 4 to afford compound 99 (89.7 mg, 50%) as a colorless solid. MS (ESI) calculated for (C₂₃H₂₁N₅OS) [M+H]⁺, 416.1; found, 416.2. ¹H NMR (400 MHz, DMSO-d₆) δ 10.79 (s, 1H), 9.49 (s, 1H), 8.71 (s, 1H), 8.54 (s, 1H), 8.33-8.25 (m, 2H), 8.09 (t, J=1.6 Hz, 1H), 7.95-7.84 (m, 3H), 7.35 (t, J=7.6 Hz, 1H), 7.17 (d, J=7.6 Hz, 1H), 4.92-4.85 (m, 1H), 3.20-3.15 (m, 1H), 1.75 (d, J=6.8 Hz, 3H), 1.00-0.90 (m, 4H).

### Example 100: N-[3-[1-(1-methylimidazol-2-yl)sulfanylethyl]phenyl]isoquinoline-3-carboxamide (100)

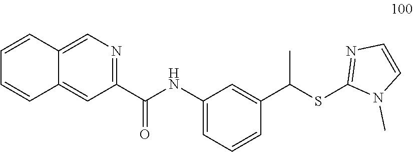 [SMILES: CC(Sc1nccn1C)c1cccc(NC(=O)c2cc3ccccc3cn2)c1]

Compound 100 (13 mg, 23% yield) was obtained according to procedures disclosed herein. ¹H NMR (500 MHz, Methanol-d4) δ 9.36 (t, J=0.9 Hz, 1H), 8.66 (s, 1H), 8.25-8.18 (m, 1H), 8.13 (dd, J=8.2, 1.1 Hz, 1H), 7.93-7.86 (m, 2H), 7.83 (ddd, J=8.1, 6.9, 1.2 Hz, 1H), 7.65 (ddd, J=8.1, 2.1, 1.0 Hz, 1H), 7.58 (ap s, 2H), 7.35 (t, J=7.9 Hz, 1H), 7.04 (dt, J=IP, 1.3 Hz, 1H), 4.77 (t, J=7.0 Hz, 1H), 3.65 (s, 3H), 1.82 (d, J=7.0 Hz, 3H). LCMS: C₂₂H₂₀N₄OS requires: 388.1, found: m/z=389 [M+H]⁺.

### Example 101: N-[2-fluoro-5-[1-(1-methylimidazol-2-yl)sulfanylethyl]phenyl]-6-(trifluoro-methyl)pyridine-2-carboxamide (101)

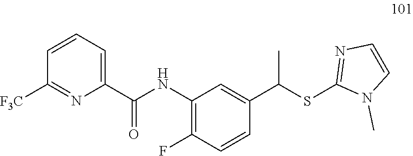 [SMILES: CC(Sc1nccn1C)c1ccc(F)c(NC(=O)c2cccc(C(F)(F)F)n2)c1]

Step 1: N-(5-acetyl-2-fluoro-phenyl)-6-(trifluoromethyl)pyridine-2-carboxamide. At rt, 1-propanephosphonic anhydride (0.35 mL, 1.18 mmol) was added to an acetonitrile (2 mL)/DMF (1 mL) solution of 1-(3-amino-4-fluoro-phenyl)ethanone (150 mg, 0.98 mmol), 6-(trifluoromethyl)picolinic acid (231 mg, 1.18 mmol), and 4-methylmorpholine (0.33 mL, 2.94 mmol). After 90 min, the reaction was diluted with EtOAc, washed with water, dried, and concentrated. This material was used without purification.

Step 2: N-[2-fluoro-5-(1-hydroxyethyl)phenyl]-6-(trifluoromethyl)pyridine-2-carboxamide. Sodium borohydride (0.07 g, 1.84 mmol) was added in portions to a methanol (9 mL) solution containing N-(5-acetyl-2-fluoro-phenyl)-6-(trifluoromethyl)pyridine-2-carboxamide (300 mg, 0.92 mmol) at 0° C. After stirring at 0° C. for 30 min, the reaction was quenched with water. The mixture was then partitioned with EtOAc, washed with brine, dried and concentrated to dryness. 274 mg cream-colored solid was obtained. The material was used without purification.

Step 3: N-[2-fluoro-5-[1-(1-methylimidazol-2-yl)sulfanylethyl]phenyl]-6-(trifluoromethyl)pyridine-2-carboxamide. At 0° C., diisopropyl azodicarboxylate (0.05 mL, 0.24 mmol) was added dropwise to a THF solution (1 mL) containing N-[2-fluoro-5-(l-hydroxyethyl)phenyl]-6-(trifluoromethyl)pyridine-2-carboxamide (40 mg, 0.12 mmol), 1-methylimidazole-2-thiol (27.8 mg, 0.24 mmol), and triphenylphosphine resin (97 mg, 0.37 mmol). The reaction was slowly warm to rt and stir for a period of 3 h. Afterwards the reaction was partitioned with water/ethyl acetate, dried and concentrated. The resulting residue was purified by HPLC to obtain the title compound (1.5 mg), ¹H NMR (500 MHz, Methanol-d4) δ 8.48 (d, J=7.9 Hz, 1H), 8.33 (t, J=7.8 Hz, 1H), 8.21 (dd, J=7.3, 2.3 Hz, 1H), 8.09 (dd, J=7.9, 1.0 Hz, 1H), 7.54-7.47 (m, 2H), 7.21 (dd, J=10.5, 8.5 Hz, 1H), 7.10 (ddd, J=8.7, 4.8, 2.4 Hz, 1H), 4.74 (q, J=7.0 Hz, 1H), 3.65 (s, 3H), 1.77 (d, J=7.0 Hz, 3H). LCMS: C₁₉H₁₆F₄N₄OS requires: 424.1, found: m/z=425 [M+H]⁺.

### Example 102: (R)-N-(4-(1-((4-methyl-4H-1,2,4-triazol-3-yl)thio)ethyl)pyridin-2-yl)-6-(trifluoromethyl)picolinamide (102a) and (S)-N-(4-(1-((4-methyl-4H-1,2,4-triazol-3-yl)thio)ethyl)pyridin-2-yl)-6-(trifluoromethyl)picolinamide (102b)

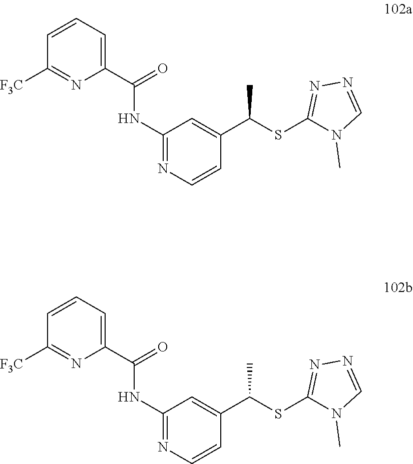 [SMILES: C[C@@H](Sc1nncn1C)c1ccnc(NC(=O)c2cccc(C(F)(F)F)n2)c1 ; C[C@H](Sc1nncn1C)c1ccnc(NC(=O)c2cccc(C(F)(F)F)n2)c1]

Followed General procedure 1-G to afford 102a and 102b.

102a: MS (ESI) calculated for (C₁₇H₁₅F₃N₆OS) [M+H]⁺, 409.4; found, 409.3. ¹H NMR (300 MHz, DMSO-d₆) δ 10.27 (s, 1H), 8.55 (s, 1H), 8.49-8.41 (m, 2H), 8.34 (d, J=5.1 Hz, 1H), 8.27-8.24 (m, 2H), 7.19-7.17 (m, 1H), 4.77 (q, J=6.9 Hz, 1H), 3.45 (s, 3H), 1.69 (d, J=6.9 Hz, 3H).

102b: MS (ESI) calculated for (C₁₇H₁₅F₃N₆OS) [M+H]⁺, 409.4; found, 409.3. ¹H NMR (300 MHz, DMSO-d₆) δ 10.27 (s, 1H), 8.55 (s, 1H), 8.47-8.35 (m, 2H), 8.34 (d, J=5.1 Hz, 1H), 8.27-8.24 (m, 2H), 7.19-7.17 (m, 1H), 4.76 (t, J=6.9 Hz, 1H), 3.45 (s, 3H), 1.69 (d, J=6.9 Hz, 3H).

### Example 103: (S)-4-methyl-N-(4-(1-((4-methyl-4H-1,2,4-triazol-3-yl)thio)ethyl)pyridin-2-yl)-6-(trifluoromethyl)picolinamide (103)

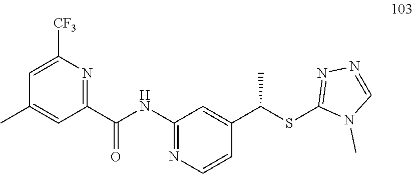 [SMILES: Cc1cc(C(=O)Nc2cc([C@H](C)Sc3nncn3C)ccn2)nc(C(F)(F)F)c1]

A suspension of C-1 (0.058 g, 0.215 mmol) in THF (0.36 mL) was cooled to 0° C. and treated with a solution of trimethylaluminum (2 M in toluene, 0.59 mL, 1.2 mmol) dropwise. After addition was complete the solution was stirred at rt for 15 min. The mixture was added dropwise to a solution of methyl 4-methyl-6-(trifluoromethyl)picolinate (0.047 g, 0.22 mmol) in THF (0.36 mL). The solution was stirred at 40° C. for 16 h. The reaction was quenched with saturated aqueous Rochelle's salt and stirred for 1 h. General Work-up Procedure 1 was followed. Compound 103 was obtained using standard flash chromatography purification methods. Yield: 0.025 g (27.9%). ¹H NMR (500 MHz, DMSO-d6) δ 10.24 (s, 1H), 8.55 (d, J=1.5 Hz, 1H), 8.36-8.30 (m, 2H), 8.24 (d, J=1.5 Hz, 1H), 8.13 (d, J=1.3 Hz, 1H), 7.18 (dd, J=5.2, 1.7 Hz, 1H), 4.77 (q, J=7.0 Hz, 1H), 3.45 (s, 3H), 2.60 (s, 3H), 1.69 (d, J=7.0 Hz, 3H). LCMS: C₁₈H₁₇F₃N₆S requires: 422, found: m/z=423 [M+H]⁺.

### Example 104: (S)-4-cyclopropyl-N-(4-(1-((4-methyl-4H-1,2,4-triazol-3-yl)thio)ethyl)pyridin-2-yl)-6-(trifluoromethyl)picolinamide (104)

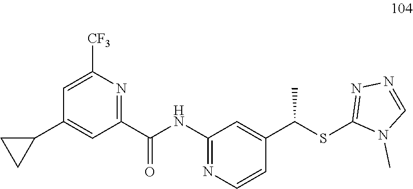 [SMILES: C[C@H](Sc1nncn1C)c1ccnc(NC(=O)c2cc(C3CC3)cc(C(F)(F)F)n2)c1]

Followed Example 103 to afford 104 (0.0112 g, 12%). ¹H NMR (500 MHz, DMSO-d₆) δ 10.23 (s, 1H), 8.56 (s, 1H), 8.33 (d, J=5.2 Hz, 1H), 8.25 (d, J=1.5 Hz, 1H), 8.12 (d, J=1.6 Hz, 1H), 7.94 (d, J=1.6 Hz, 1H), 7.18 (dd, J=5.2, 1.6 Hz, 1H), 4.77 (q, J=7.0 Hz, 1H), 3.44 (s, 3H), 2.33 (tt, J=8.4, 4.9 Hz, 1H), 1.68 (d, J=7.0 Hz, 3H), 1.29-1.18 (m, 2H), 1.11-1.04 (m, 2H). LCMS: C₂₀H₁₉F₃N₆OS requires: 448.1, found: m/z=449.2 [M+H]⁺.

### Example 105: (S)-4-chloro-N-(4-(1-((4-methyl-4H-1,2,4-triazol-3-yl)thio)ethyl)pyridin-2-yl)-6-(trifluoromethyl)picolinamide (105)

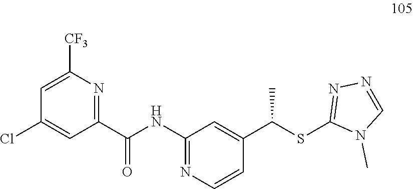 [SMILES: C[C@H](Sc1nncn1C)c1ccnc(NC(=O)c2cc(Cl)cc(C(F)(F)F)n2)c1]

Followed Example 103 to afford 105 (0.072 g, 44%). ¹H NMR (500 MHz, DMSO-d₆) δ 10.29 (s, 1H), 8.55 (s, 1H), 8.51 (d, J=1.8 Hz, 1H), 8.48 (d, J=1.8 Hz, 1H), 8.34 (d, J=5.1 Hz, 1H), 8.22 (d, J=1.4 Hz, 1H), 7.19 (dd, J=5.2, 1.7 Hz, 1H), 4.77 (q, J=7.0 Hz, 1H), 3.45 (s, 3H), 1.68 (d, J=7.0 Hz, 3H). LCMS: C₁₇H₁₄C₁F₃N₆OS requires: 442.1, found: m/z=443 [M+H]⁺.

### Example 106: (S)-5-chloro-N-(4-(1-((4-methyl-4H-1,2,4-triazol-3-yl)thio)ethyl)pyridin-2-yl)-6-(trifluoromethyl)picolinamide (106)

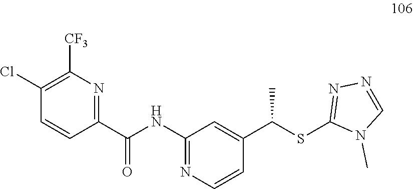 [SMILES: C[C@H](Sc1nncn1C)c1ccnc(NC(=O)c2ccc(Cl)c(C(F)(F)F)n2)c1]

Followed Example 108 to afford 106 (23 mg, 24%) as a colorless solid. MS (ESI) calc'd for (C₁₇H₁₄ClF₃N₆OS) [M+H]⁺, 443.1; found, 443.5. ¹H NMR (400 MHz, DMSO-d₆) δ 10.22 (s, 1H), 8.56-8.53 (m, 2H), 8.43 (d, J=8.4 Hz, 1H), 8.33 (d, J=5.2 Hz, 1H), 8.25-8.17 (m, 1H), 7.19-7.17 (m, 1H), 4.79-4.73 (m, 1H), 3.44 (s, 3H), 1.68 (d, J=7.2 Hz, 3H).

### Example 107: (S)-8-fluoro-N-(4-(1-((4-methyl-4H-1,2,4-triazol-3-yl)thio)ethyl)pyridin-2-yl)quinoline-2-carboxamide (107)

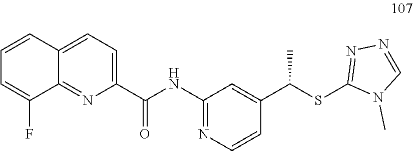 [SMILES: C[C@H](Sc1nncn1C)c1ccnc(NC(=O)c2ccc3cccc(F)c3n2)c1]

Followed Example 108 to afford 107. Yield: 19 mg (50%). ¹H NMR (500 MHz, DMSO-d₆) δ 10.54 (s, 1H), 8.78 (dd, J=8.7, 1.5 Hz, 1H), 8.54 (s, 1H), 8.38 (d, J=8.5 Hz, 1H), 8.34 (dd, J=5.2, 0.8 Hz, 1H), 8.33-8.29 (m, 1H), 8.03-7.97 (m, 1H), 7.82-7.76 (m, 2H), 7.17 (dd, J=5.2, 1.6 Hz, 1H), 4.77 (q, J=7.0 Hz, 1H), 3.44 (s, 3H), 1.69 (d, J=7.0 Hz, 3H). LCMS: C₂₀H₁₇FN₆OS requires: 408.5, found: m/z 409.4 [M+H]⁺.

### Example 108: (S)-N-(4-(1-((4-methyl-4H-1,2,4-triazol-3-yl)thio)ethyl)pyridin-2-yl)-5,6,7,8-tetrahydroquinoline-2-carboxamide (108)

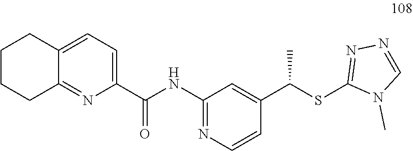 [SMILES: C[C@H](Sc1nncn1C)c1ccnc(NC(=O)c2ccc3c(n2)CCCC3)c1]

At ambient temperature, C-1.HCl (21 mg, 0.076 mmol), 5,6,7,8-tetrahydroquinoline-2-carboxylic acid (17 mg, 0.097 mmol) and HATU (37 mg, 0.098 mmol) were dissolved in N,N-dimethylformamide (0.2 mL). N,N-Diisopropylethylamine (41 μL. 0.24 mmol) was added and the reaction was stirred for 1 h at 55° C. Dimethylformamide was removed under reduced pressure and the crude reaction mixture was purified by flash column chromatography to give the title compound (28 mg, 91%). ¹H NMR (500 MHz, DMSO-d₆) δ 10.38 (s, 1H), 8.53 (s, 1H), 8.28 (d, J=5.2 Hz, 1H), 8.25 (d, J=1.5 Hz, 1H), 7.93 (d, J=7.8 Hz, 1H), 7.76 (t, J=8.0 Hz, 1H), 7.12 (dd, J=5.2, 1.6 Hz, 1H), 4.73 (q, J=7.0 Hz, 1H), 3.43 (s, 3H), 2.95 (t, J=6.4 Hz, 1H), 2.90-2.82 (m, 2H), 2.80 (t, J=6.3 Hz, 1H), 1.93-1.69 (m, 4H), 1.66 (d, J=7.0 Hz, 3H). LCMS: C₂₀H₂₂N₆OS requires: 394.2, found: m/z 395.4 [M+H]⁺.

### Example 109: (S)-N-(4-(1-((4-methyl-4H-1,2,4-triazol-3-yl)thio)ethyl)pyridin-2-yl)-5,6,7,8-tetrahydroisoquinoline-3-carboxamide (109)

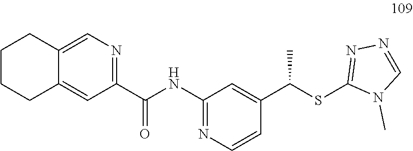 [SMILES: C[C@H](Sc1nncn1C)c1ccnc(NC(=O)c2cc3c(cn2)CCCC3)c1]

Followed Example 108 to afford 109 in 22 mg (70%) yield. ¹H NMR (500 MHz, DMSO-d₆) δ 10.37 (s, 1H), 8.53 (s, 1H), 8.42 (s, 1H), 8.28 (dd, J=5.1, 0.7 Hz, 1H), 8.23 (d, J=1.5 Hz, 1H), 7.90 (s, 1H), 7.12 (dd, J=5.2, 1.6 Hz, 1H), 4.74 (q, J=7.0 Hz, 1H), 3.44 (s, 3H), 2.93-2.80 (m, 4H), 1.82-1.71 (m, 4H), 1.66 (d, J=7.0 Hz, 3H). LCMS: C₂₀H₂₂N₆OS requires: 394.2, found: m/z 395.4 [M+H]⁺.

### Example 110: (S)-6-(1,1-difluoroethyl)-V-(4-(1-((4-methyl-4H-1,2,4-triazol-3-yl)thio)ethyl)pyridin-2-yl)picolinamide (110)

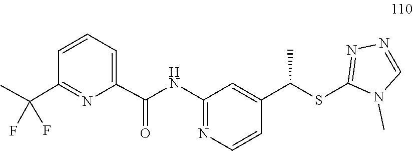 [SMILES: C[C@H](Sc1nncn1C)c1ccnc(NC(=O)c2cccc(C(C)(F)F)n2)c1]

Followed Example 108 to afford the title compound as a trifluoroacetate salt. Yield: 17 mg (42%). ¹H NMR (500 MHz, DMSO-d₆) δ 10.40 (s, 1H), 8.58 (s, 1H), 8.39-8.27 (m, 3H), 8.25 (d, J=1.5 Hz, 1H), 8.03 (dd, J=7.2, 1.6 Hz, 1H), 7.17 (dd, J=5.2, 1.6 Hz, 1H), 4.77 (q, J=7.0 Hz, 1H), 3.45 (s, 3H), 2.14 (t, J=19.3 Hz, 3H), 1.68 (d, J=7.0 Hz, 3H). LCMS: C₁₈H₁₈F₂OS requires: 404.1, found: m/z 405.4 [M+H]⁺.

### Example 111: (S)-N-(4-(1-((4-methyl-4H-1,2,4-triazol-3-yl)thio)ethyl)pyridin-2-yl)-4-(trifluoromethyl)quinoline-2-carboxamide (111)

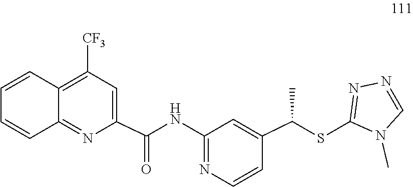 [SMILES: C[C@H](Sc1nncn1C)c1ccnc(NC(=O)c2cc(C(F)(F)F)c3ccccc3n2)c1]

Followed Example 108 to afford 111 as a trifluoroacetate salt. Yield: 15 mg (30%). ¹H NMR (500 MHz, DMSO-d₆) δ 10.66 (s, 1H), 8.57 (s, 1H), 8.53 (s, 1H), 8.48 (dd, J=8.5, 1.2 Hz, 1H), 8.36 (d, J=5.1 Hz, 1H), 8.31-8.28 (m, 1H), 8.24 (d, J=8.5 Hz, 1H), 8.10 (ddd, J=8.3, 7.0, 1.4 Hz, 1H), 8.01 (ddd, J=8.4, 6.9, 1.4 Hz, 1H), 7.20 (dd, J=5.1, 1.6 Hz, 1H), 4.78 (q, J=7.1 Hz, 1H), 3.46 (s, 3H), 1.69 (d, J=7.0 Hz, 3H). LCMS: C₂₁H₁₇F₃N₆OS requires: 458.1, found: m/z 459.4 [M+H]⁺.

### Example 112: (S)-N-(4-(1-((4-methyl-4H-1,2,4-triazol-3-yl)thio)ethyl)pyridin-2-yl)quinoline-2-carboxamide (112)

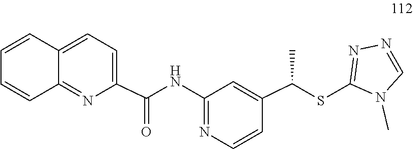 [SMILES: C[C@H](Sc1nncn1C)c1ccnc(NC(=O)c2ccc3ccccc3n2)c1]

Followed Example 108 with stirring at 20° C. for 1.5 h to afford 112 as a light yellow solid. MS (ESI) calculated for (C₂₀H₁₈N₆OS) [M+H]⁺, 391.1; found, 391.1. ¹H NMR (400 MHz, DMSO-d₆) δ 10.67 (s, 1H), 8.71 (d, J=8.4 Hz, 1H), 8.56 (s, 1H), 8.36-8.32 (m, 4H), 8.17-8.16 (m, 1H), 7.97-7.95 (m, 1H), 7.80-7.77 (m, 1H), 7.19-7.17 (m, 1H), 4.78 (q, J=7.2 Hz, 1H), 3.47 (s, 3H), 1.70 (d, J=7.2 Hz, 3H).

### Example 113: (S)-6-cyclopropyl-5-methyl-N-(4-(1-((4-methyl-4H-1,2,4-triazol-3-yl)thio)ethyl)pyridin-2-yl)picolinamide (113)

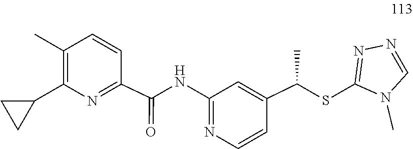 [SMILES: Cc1ccc(C(=O)Nc2cc([C@H](C)Sc3nncn3C)ccn2)nc1C1CC1]

To a stirred solution of C-1.HCl (100 mg, 0.37 mmol) and 6-cyclopropyl-5-methylpicolinic acid (98 mg, 0.55 mmol) in pyridine (1 mL) and EtOAc (1 mL) was added propylphosphonic anhydride (396 mg, 50% w/w in EtOAc, 0.72 mmol). The solution was stirred at 60° C. for 3 h. The reaction was quenched by the addition of water (10 mL). General Work-up Procedure 1 was followed. Compound 113 (23.4 mg, 11%) was obtained as a colorless solid using standard HPLC purification methods. MS (ESI) calc'd for (C₂₀H₂₂N₆OS) [M+H]⁺, 395.1; found, 395.0. ¹H NMR (300 MHz, Methanol-d₄) δ 8.48 (s, 1H), 8.32-8.26 (m, 2H), 7.92 (d, J=7.8 Hz, 1H), 7.75-7.72 (m, 1H), 7.14-7.11 (m, 1H), 4.75 (q, J=7.2 Hz, 1H), 3.55 (s, 3H), 2.56 (s, 3H), 2.34-2.30 (m, 1H), 1.79 (d, J=7.2 Hz, 3H), 1.26-1.21 (m, 2H), 1.19-1.08 (m, 2H).

### Example 114: (S)-5-chloro-N-(4-(1-((4-methyl-4H-1,2,4-triazol-3-yl)thio)ethyl)pyridin-2-yl)-4-(trifluoromethyl)picolinamide (114)

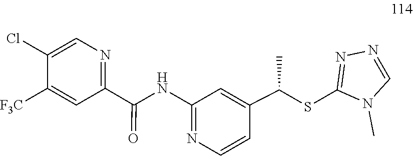 [SMILES: C[C@H](Sc1nncn1C)c1ccnc(NC(=O)c2cc(C(F)(F)F)c(Cl)cn2)c1]

Followed Example 108 to give Compound 114 (13.2 mg, 7%) as a colorless solid. MS (ESI) calc'd for (C₁₇H₁₄ClF₃N₆OS) [M+H]⁺, 443.1; found, 443.1. ¹H NMR (300 MHz, DMSO-d₆) δ 10.39 (s, 1H), 9.15 (s, 1H), 8.54 (s, 1H), 8.42 (s, 1H), 8.34-8.32 (m, 1H), 8.22 (s, 1H), 7.19-7.17 (m, 1H), 4.80-4.73 (m, 1H), 3.45 (s, 3H), 1.67 (d, J=6.9 Hz, 3H).

### Example 115: (S)-N-(4-(1-((4-methyl-4H-1,2,4-triazol-3-yl)thio)ethyl)pyridin-2-yl)benzo[d]isoxazole-3-carboxamide (115)

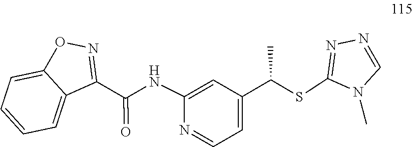 [SMILES: C[C@H](Sc1nncn1C)c1ccnc(NC(=O)c2noc3ccccc23)c1]

Followed General procedure 1-G using C-1 to provide the title compound. Yield: 7.5 mg (10%). ¹H NMR (500 MHz, Methanol-d₄) δ 8.72 (d, J=3.8 Hz, 1H), 8.32 (dd, J=5.3, 0.8 Hz, 1H), 8.26 (dt, J=8.0, 1.1 Hz, 1H), 8.19-8.15 (m, 1H), 7.80-7.69 (m, 2H), 7.53 (ddd, J=7.9, 6.9, 0.9 Hz, 1H), 7.27 (dt, J=5.4, 1.4 Hz, 1H), 4.84 (q, J=7.1 Hz, 1H), 3.60 (s, 3H), 1.80 (d, J=7.0 Hz, 3H). LCMS: C₁₈H₁₆N₆O₂S requires: 380.1, found: m/z=381.1 [M+H]⁺.

### Example 116: N-[4-[(1S)-1-[(4-methyl-1,2,4-triazol-3-yl)sulfanyl]ethyl]-2-pyridyl]-3-(pentafluoro-λ⁶-sulfanyl)benzamide (116)

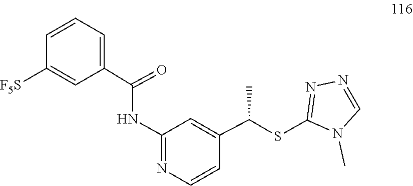

Followed General procedure 1-G with reaction mixture maintained at rt for 7 d to afford the title compound (26 mg, 41%) as a colorless solid. ¹H NMR (500 MHz, DMSO-d₆) δ 11.35 (s, 1H), 8.74 (s, 1H), 8.50 (t, J=2.0 Hz, 1H), 8.35 (d, J=5.2 Hz, 1H), 8.33-8.27 (m, 1H), 8.22-8.10 (m, 2H), 7.79 (t, J=8.0 Hz, 1H), 7.18 (dd, J=5.2, 1.6 Hz, 1H), 4.77 (q, J=7.0 Hz, 1H), 3.50 (s, 3H), 1.68 (d, J=7.0 Hz, 3H). LCMS: C₁₇H₁₆F₅N₅OS₂ requires: 465.1, found: m/z=466 [M+H]⁺.

### Example 117: 6-cyclopropyl-N-[4-[(1S)-1-[(4-methyl-1,2,4-triazol-3-yl)sulfanyl]ethyl]-2-pyridyl]pyridine-2-carboxamide (117)

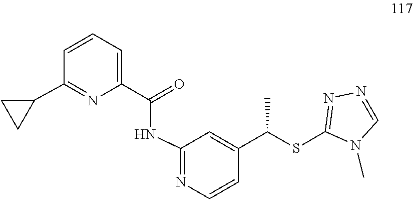 [SMILES: C[C@H](Sc1nncn1C)c1ccnc(NC(=O)c2cccc(C3CC3)n2)c1]

Followed General procedure 1-G to afford the title compound (8 mg, 15%) as a colorless solid. ¹H NMR (500 MHz, DMSO-d₆) δ 10.39 (s, 1H), 8.70 (s, 1H), 8.33 (d, J=5.2 Hz, 1H), 8.25 (d, J=1.5 Hz, 1H), 7.99-7.93 (m, 2H), 7.64-7.59 (m, 1H), 7.18 (dd, J=5.2, 1.7 Hz, 1H), 4.78 (q, J=7.0 Hz, 1H), 3.48 (s, 3H), 2.27 (tt, J=7.8, 5.0 Hz, 1H), 1.68 (d, J=7.0 Hz, 3H), 1.12-1.03 (m, 4H). LCMS: C₁₉H₂₀N₆OS requires: 380.1, found: m/z=381 [M+H]⁺.

### Example 118: N-[4-[(1S)-1-[(4-methyl-1,2,4-triazol-3-yl)sulfanyl]ethyl]-2-pyridyl]-2-(trifluoromethyl)thiazole-4-carboxamide (118)

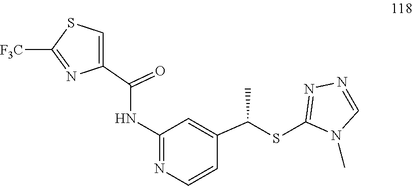 [SMILES: C[C@H](Sc1nncn1C)c1ccnc(NC(=O)c2csc(C(F)(F)F)n2)c1]

Followed General procedure 1-G to afford the title compound (4 mg, 7%) as a colorless solid. ¹H NMR (500 MHz, Methanol-d₄) δ 9.50 (s, 1H), 8.96 (s, 1H), 8.46-8.39 (m, 1H), 8.19 (d, J=1.7 Hz, 1H), 7.74 (dd, J=6.4, 1.8 Hz, 1H), 5.12 (q, J=7.1 Hz, 1H), 3.78 (s, 3H), 1.88 (d, J=12 Hz, 3H). LCMS: C₁₅H₁₃F₃N₆OS₂ requires: 414.1, found: m/z=415 [M+H]⁺.

### Example 119: 4-bromo-N-[4-[(1S)-1-[(4-methyl-1,2,4-triazol-3-yl)sulfanyl]ethyl]-2-pyridyl]-6-(trifluoromethyl)pyridine-2-carboxamide (119)

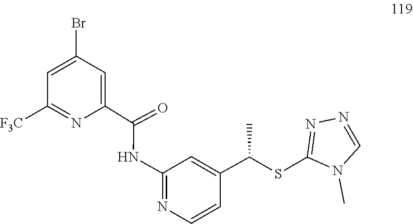 [SMILES: C[C@H](Sc1nncn1C)c1ccnc(NC(=O)c2cc(Br)cc(C(F)(F)F)n2)c1]

To a suspension of C-1·HCl (0.2 g, 0.72 mmol, 1 eq.) in THF (2.0 mL) at 0° C. was added trimethyl aluminum (1.2 mL, 2.0 M in hexanes, 2.4 mmol, 3.3 eq) dropwise. The mixture was stirred until a clear solution resulted (40 min). This solution was then added dropwise to a solution of methyl 4-bromo-6-(trifluoromethyl)pyridine-2-carboxylate (0.21 g, 0.75 mmol, 1.1 eq.) and THF (2 mL). The mixture was heated at 40° C. for 18 h. The solution was then added dropwise to saturated aqueous Rochelle's salt (3.0 mL), EtOAc was added (5 mL) and the mixture stirred until two clear phases resulted. The phases were separated, and the aqueous extracted with EtOAc (2×5 mL). The combined organic phases were dried and filtered. Compound 119 (80 mg, 23%) was obtained as a colorless solid using standard flash chromatography purification methods. ¹H NMR (500 MHz, DMSO-d₆) δ 10.27 (s, 1H), 8.59 (q, J=1.7 Hz, 2H), 8.54 (s, 1H), 8.33 (dd, J=5.2, 0.8 Hz, 1H), 8.23-8.17 (m, 1H), 7.18 (dd, J=5.2, 1.6 Hz, 1H), 4.76 (q, J=6.9 Hz, 1H), 3.44 (s, 3H), 1.67 (d, J=7.0 Hz, 3H); LCMS: C₁₇H₁₄BrF₃N₆OS requires: 486.0, found: m/z=486 [M+H]⁺.

### Example 120: N-[4-[(1S)-1-[(4-methyl-1,2,4-triazol-3-yl)sulfanyl]ethyl]-2-pyridyl]-4-(trifluoromethyl)-1H-indole-2-carboxamide (120)

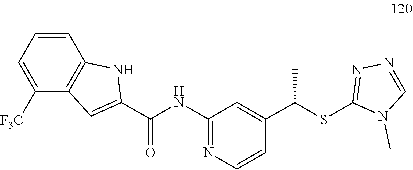 [SMILES: C[C@H](Sc1nncn1C)c1ccnc(NC(=O)c2cc3c(C(F)(F)F)cccc3[nH]2)c1]

Followed Example 119 to afford the title compound (17 mg, 7%) as a colorless solid: ¹H NMR (500 MHz, DMSO-d₆) δ 12.30 (d, J=2.1 Hz, 1H), 11.16 (s, 1H), 8.58 (s, 1H), 8.34 (d, J=5.2 Hz, 1H), 8.21 (d, J=1.4 Hz, 1H), 7.91 (s, 1H), 7.78 (d, J=8.2 Hz, 1H), 7.53-7.45 (m, 1H), 7.41 (t, J=7.7 Hz, 1H), 7.13 (dd, J=5.2, 1.7 Hz, 1H), 4.74 (q, J=7.0 Hz, 1H), 3.46 (s, 3H), 1.68 (d, J=7.0 Hz, 3H); LCMS: C₂₀H₁₇F₃N₆OS requires: 446.1, found: m/z=447 [M+H]⁺.

### Example 121: N-[4-[(1S)-1-[(4-methyl-1,2,4-triazol-3-yl)sulfanyl]ethyl]-2-pyridyl]-1,3-benzothiazole-2-carboxamide (121)

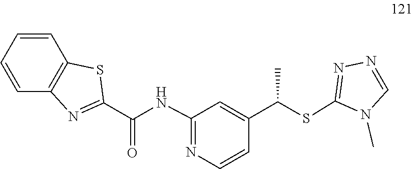 [SMILES: C[C@H](Sc1nncn1C)c1ccnc(NC(=O)c2nc3ccccc3s2)c1]

Compound 121 (18 mg) was obtained according to procedures disclosed herein as a colorless solid: ¹H NMR (500 MHz, DMSO-d₆) δ 10.48 (s, 1H), 8.60 (s, 1H), 8.40-8.33 (m, 1H), 8.33-8.27 (m, 1H), 8.27-8.21 (m, 1H), 8.13 (d, J=1.5 Hz, 1H), 7.72-7.62 (m, 2H), 7.20 (dd, J=5.2, 1.6 Hz, 1H), 4.77 (q, J=7.0 Hz, 1H), 3.46 (s, 3H), 1.68 (d, J=7.0 Hz, 3H); LCMS: C₁₈H₁₆N₆OS₂ requires: 396.1, found: m/z=397 [M+H]⁺.

### Example 122: (S)-N-(4-(1-((4-methyl-4H-1,2,4-triazol-3-yl)thio)ethyl)pyridin-2-yl)-6-(trifluoromethoxy)picolinamide (122)

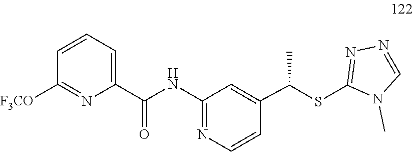 [SMILES: C[C@H](Sc1nncn1C)c1ccnc(NC(=O)c2cccc(OC(F)(F)F)n2)c1]

C-1·HCl (21 mg, 0.077 mmol) was dissolved in EtOAc (0.2 mL) and N,N-dimethylformamide (0.2 mL) mixture at ambient temperature. 6-(Trifluoromethoxy)picolinic acid (19 mg, 0.092 mmol) was added, followed by propylphosphonic anhydride solution in ethyl acetate (0.1 mL, 1.47 M) and pyridine (35 μL, 0.44 mmol). After 2 h stirring at ambient temperature and 55° C. overnight, HATU (32 mg, 0.085 mmol) and N,N-diisopropyl ethylamine (35 μL, 0.20 mmol) was added and the reaction was further stirred at 55° C. After several hours, the reaction was cooled to ambient temperature and stirred for 2 days. The reaction was diluted with DCM and 1 N hydrochloric acid was added. The layers were separated and the aqueous layer was extracted with chloroform:isopropyl alcohol (2:1) mixture twice. The combined organic layers were dried and concentrated. Purification on flash column chromatography afforded the title compound as an off-white solid. Yield: 7.8 mg (20%). ¹H NMR (500 MHz, DMSO-d₆) δ 9.96 (s, 1H), 8.53 (s, 1H), 8.39-8.29 (m, 2H), 8.26-8.17 (m, 2H), 7.65 (dd, J=8.2, 0.7 Hz, 1H), 7.16 (dd, J=5.2, 1.6 Hz, 1H), 4.75 (q, J=7.0 Hz, 1H), 3.43 (s, 3H), 1.66 (d, J=7.0 Hz, 3H). LCMS: C₁₇H₁₅F₃N₆O₂S requires: 424.1, found: m/z 425.4 [M+H]⁺.

### Example 123: (S)-6-ethyl-N-(4-(1-((4-methyl-4H-1,2,4-triazol-3-yl)thio)ethyl)pyridin-2-yl)picolinamide (123)

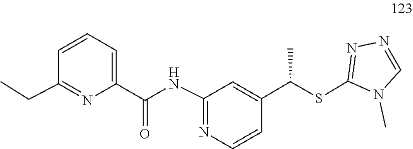 [SMILES: CCc1cccc(C(=O)Nc2cc([C@H](C)Sc3nncn3C)ccn2)n1]

Compound 123 was obtained according to procedures disclosed herein as a trifluoroacetate. Yield: 6.6 mg (18%). ¹H NMR (500 MHz, DMSO-d₆) δ 10.47 (s, 1H), 8.55 (s, 1H), 8.30 (d, J=5.1 Hz, 1H), 8.26 (d, J=1.5 Hz, 1H), 8.07-7.96 (m, 2H), 7.61 (dd, J=5.8, 3.0 Hz, 1H), 7.14 (dd, J=5.2, 1.7 Hz, 1H), 4.74 (q, J=6.9 Hz, 1H), 3.44 (s, 3H), 2.90 (q, J=7.6 Hz, 2H), 1.67 (d, J=7.0 Hz, 3H), 1.31 (t, J=7.6 Hz, 3H). LCMS: C₁₈H₂₀N₆OS requires: 368.1, found: m/z 369.4 [M+H]⁺.

### Example 124: 6-chloro-N-[4-[(LV)-1-[(4-methyl-1,2,4-triazol-3-yl)sulfanyl]ethyl]-2-pyridyl]pyridine-2-carboxamide (124)

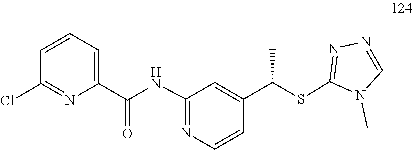 [SMILES: C[C@H](Sc1nncn1C)c1ccnc(NC(=O)c2cccc(Cl)n2)c1]

Followed Example 117 to afford compound 124 (10 mg, 0.03 mmol, 25% yield) as a colorless solid. ¹H NMR (500 MHz, Methanol-d₄) δ 8.74 (s, 1H), 8.32 (d, J=5.6 Hz, 1H), 8.28-8.19 (m, 2H), 8.07 (t, J=7.8 Hz, 1H), 7.74 (dd, J=8.0, 0.9 Hz, 1H), 7.32 (dd, J=5.6, 1.7 Hz, 1H), 4.83 (t, J=7.1 Hz, 1H), 3.62 (s, 3H), 1.80 (d, J=7.1 Hz, 3H). LCMS: C₁₆H₁₅ClN₆OS requires: 374.1, found: m/z=375.1 [M+H]⁺

### Example 125: (S)-5-chloro-6-methoxy-N-(4-(1-((4-methyl-4H-1,2,4-triazol-3-yl)thio)ethyl)pyridin-2-yl)picolinamide (125)

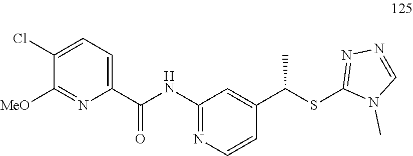 [SMILES: COc1nc(C(=O)Nc2cc([C@H](C)Sc3nncn3C)ccn2)ccc1Cl]

Followed Example 122 to afford compound 125 (7.8 mg, 23%). ¹H NMR (500 MHz, DMSO-d₆) δ 10.24 (s, 1H), 8.53 (s, 1H), 8.31 (dd, J=5.1, 0.7 Hz, 1H), 8.25-8.20 (m, 1H), 8.16 (d, J=7.9 Hz, 1H), 7.78 (d, J=7.9 Hz, 1H), 7.14 (dd, J=5.2, 1.6 Hz, 1H), 4.74 (q, J=7.0 Hz, 1H), 4.12 (s, 3H), 3.44 (s, 3H), 1.66 (d, J=7.0 Hz, 3H). LCMS: C₁₇H₁₇ClN₆O₂S requires: 404.1, found: m/z 405.3 [M+H]⁺.

### Example 126: 4-[(3S)-3-hydroxypyrrolidin-1-yl]-N-[4-[(1S)-1-[(4-methyl-1,2,4-triazol-3-yl)sulfanyl]ethyl]-2-pyridyl]-6-(trifluoromethyl)pyridine-2-carboxamide (126)

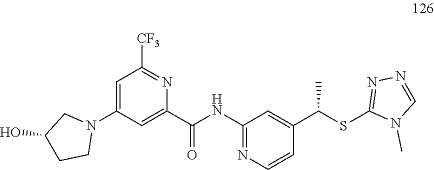 [SMILES: C[C@H](Sc1nncn1C)c1ccnc(NC(=O)c2cc(N3CC[C@H](O)C3)cc(C(F)(F)F)n2)c1]

Compound 126 (19 mg, 45%) was obtained according to procedures disclosed herein as a colorless solid: 1H NMR (500 MHz, DMSO-d₆) δ 10.25 (s, 1H), 8.60 (s, 1H), 8.31 (d, J=5.0 Hz, 1H), 8.25 (t, J=1.0 Hz, 1H), 7.38 (s, 1H), 7.16 (dd, J=5.2, 1.6 Hz, 1H), 7.07 (s, 1H), 4.76 (q, J=7.0 Hz, 1H), 4.51-4.39 (m, 1H), 3.63-3.48 (m, 3H), 3.44 (s, 3H), 3.33 (d, J=11.0 Hz, 1H), 2.14-2.03 (m, 1H), 2.03-1.92 (m, 1H), 1.68 (d, J=7.0 Hz, 3H); LCMS: C₂₁H₂₂F₃N₇O₂S requires: 493.2, found: m/z=494 [M+H]⁺.

### Example 127: N-[5-[1-[(4-methyl-1,2,4-triazol-3-yl)sulfanyl]ethyl]-3-pyridyl]-6-(trifluoromethyl)pyridine-2-carboxamide (127)

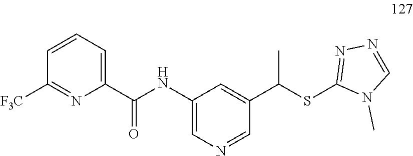 [SMILES: CC(Sc1nncn1C)c1cncc(NC(=O)c2cccc(C(F)(F)F)n2)c1]

Compound 127 was obtained according to procedures disclosed herein (105 mg, 0.26 mmol, 36% yield). ¹H NMR (500 MHz, DMSO-d₆) δ 10.69 (s, 1H), 8.96 (d, J=2.3 Hz, 1H), 8.55 (s, 1H), 8.40 (dt, J=15.5, 7.8 Hz, 2H), 8.34-8.13 (m, 3H), 4.79 (q, J=7.0 Hz, 1H), 3.43 (s, 3H), 1.70 (d, J=7.0 Hz, 3H). LCMS: C₁₇H₁₅F₃N₆OS requires: 408.1 found: m/z=409.2 [M+H]⁺

### Example 128: (S)-N-(4-(1-((4-methyl-1H-pyrazol-3-yl)thio)ethyl)pyridin-2-yl)quinoline-2-carboxamide (128a) and (R)-N-(4-(1-((4-methyl-1H-pyrazol-3-yl)thio)ethyl)pyridin-2-yl)quinoline-2-carboxamide (128b)

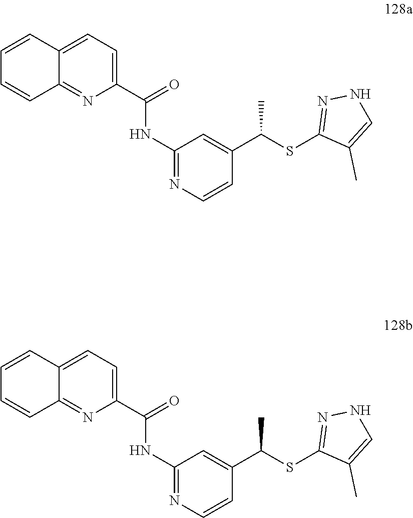 [SMILES: Cc1c[nH]nc1S[C@@H](C)c1ccnc(NC(=O)c2ccc3ccccc3n2)c1 ; Cc1c[nH]nc1S[C@H](C)c1ccnc(NC(=O)c2ccc3ccccc3n2)c1]

Compounds 128b (33.1 mg, 39%) and 128a (34.3 mg, 40%) were obtained according to procedures disclosed herein.

128b: MS (ESI) calculated for (C₂₁H₁₉N₅OS) [M+H]⁺, 390.1, found, 390.0. ¹H NMR (400 MHz, DMSO-d₆) δ 12.79 (s, 1H), 10.62 (s, 1H), 8.70 (d, J=8.4 Hz, 1H), 8.32-8.26 (m, 4H), 8.16 (d, J=8.0 Hz, 1H), 7.96-7.92 (m, 1H), 7.81-7.77 (m, 1H), 7.45 (s, 1H), 7.12-7.05 (m, 1H), 4.40-4.35 (m, 1H), 1.83 (s, 3H), 1.58 (d, J=7.2 Hz, 3H).

128a: MS (ESI) calculated for (C₂₁H₁₉N₅OS) [M+H]⁺, 390.1, found, 390.0. ¹H NMR (400 MHz, DMSO-d₆) δ 12.79 (s, 1H), 10.62 (s, 1H), 8.70 (d, J=8.4 Hz, 1H), 8.32-8.26 (m, 4H), 8.16 (d, J=8.0 Hz, 1H), 7.96-7.92 (m, 1H), 7.81-7.77 (m, 1H), 7.45 (s, 1H), 7.12-7.05 (m, 1H), 4.40-4.35 (m, 1H), 1.83 (s, 3H), 1.58 (d, J=7.2 Hz, 3H).

### Example 129: N-(4-(1-((1,4-dimethyl-1H-imidazol-2-yl)thio)ethyl)pyridin-2-yl)quinoline-2-carboxamide (129)

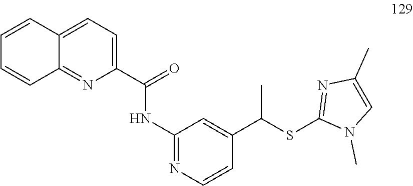 [SMILES: Cc1cn(C)c(SC(C)c2ccnc(NC(=O)c3ccc4ccccc4n3)c2)n1]

Compound 129 (150 mg, 64%) was obtained according to procedures disclosed herein as a yellow solid. MS (ESI) calc'd for (C₂₂H₂₁N₅OS) [M+H]⁺, 404.2; found, 404.2. ¹H NMR (400 MHz, DMSO-d₆) δ 10.63 (s, 1H), 8.71 (d, J=8.8 Hz, 1H), 8.32-8.27 (m, 4H), 8.16 (d, J=8.0 Hz, 1H), 7.94 (t, J=7.2 Hz, 1H), 7.79 (t, J=7.2 Hz, 1H), 7.10-7.09 (m, 1H), 6.92 (s, 1H), 4.56 (q, J=6.8 Hz, 1H), 3.34 (s, 3H), 2.08 (s, 3H), 1.63 (d, J=6.8 Hz, 3H).

### Example 130: (S)-N-(2-methyl-5-(1-((4-methyl-4H-1,2,4-triazol-3-yl)thio)ethyl)benzo[d]oxazol-7-yl)-6-(trifluoromethyl)picolinamide (130a) and (R)-N-(2-methyl-5-(1-((4-methyl-4H-1,2,4-triazol-3-yl)thio)ethyl)benzo[d]oxazol-7-yl)-6-(trifluoromethyl)picolinamide (130b)

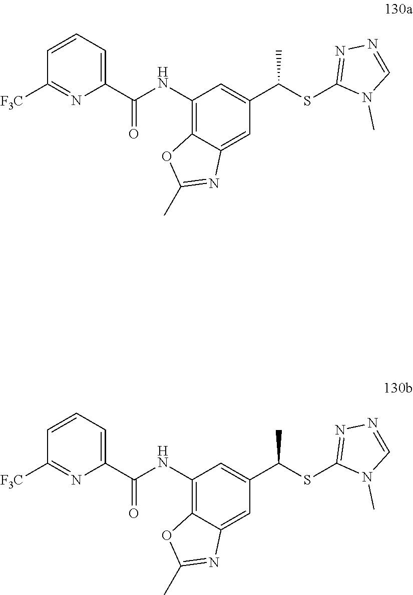 [SMILES: Cc1nc2cc([C@H](C)Sc3nncn3C)cc(NC(=O)c3cccc(C(F)(F)F)n3)c2o1 ; Cc1nc2cc([C@@H](C)Sc3nncn3C)cc(NC(=O)c3cccc(C(F)(F)F)n3)c2o1]

Followed General procedure 1-G using B-1 to afford N-(2-methyl-5-[1-[(4-methyl-4H-1,2,4-triazol-3-yl)sulfanyl]ethyl]-1,3-benzoxazol-7-yl)-6-(trifluoromethyl)pyridine-2-carboxamide (90 mg, 40%) as a yellow solid. The racemic product was separated by chiral-prep-HPLC to afford 130a (20.6 mg) with shorter retention time as a light yellow solid and 130b (25.0 mg) with longer retention time as a light yellow solid.

130a: MS (ESI) calc'd for (C₂₀H₁₇F₃N₆O₂S) [M+H]⁺, 463.1; found, 463.1. ¹H NMR (300 MHz, DMSO-d₆) δ 10.65 (s, 1H), 8.52 (s, 1H), 8.46-8.37 (m, 2H), 8.25-8.22 (m, 1H), 7.73 (d, J=1.2 Hz, 1H), 7.44 (d, J=1.2 Hz, 1H), 4.80 (q, J=6.9 Hz, 1H), 3.36 (s, 3H), 2.62 (s, 3H), 1.68 (d, J=6.9 Hz, 3H).

130b: MS (ESI) calc'd for (C₂₀H₁₇F₃N₆O₂S) [M+H]⁺, 463.1; found, 463.1. ¹H NMR (300 MHz, DMSO-d₆) δ 10.65 (s, 1H), 8.52 (s, 1H), 8.46-8.37 (m, 2H), 8.25-8.22 (m, 1H), 7.73 (d, J=1.2 Hz, 1H), 7.44 (d, J=1.2 Hz, 1H), 4.80 (q, J=6.9 Hz, 1H), 3.36 (s, 3H), 2.62 (s, 3H), 1.68 (d, J=6.9 Hz, 3H).

### Example 131: N-(1-hydroxy-1-methyl-6-(1-(4-methyl-4H-1,2,4-triazol-3-ylthio)ethyl)-2,3-dihydro-1H-inden-4-yl)quinoline-2-carboxamide (131)

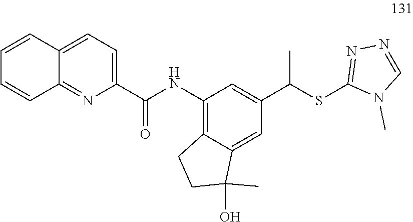 [SMILES: CC(Sc1nncn1C)c1cc(NC(=O)c2ccc3ccccc3n2)c2c(c1)C(C)(O)CC2]

Compound 131 (83.7 mg, 6%) was obtained according to procedures disclosed herein as a colorless solid. MS (ESI) calc'd for (C₂₅H₂₅N₅O₂S) [M+H]⁺, 460.2; found, 460.0. ¹H NMR (300 MHz, DMSO-d₆) δ 10.43 (s, 1H), 8.68 (d, J=8.4 Hz, 1H), 8.55 (d, J=3.3 Hz, 1H), 8.29-8.22 (m, 2H), 8.15 (d, J=8.1 Hz, 1H), 8.00-7.88 (m, 2H), 7.79 (t, J=7.2 Hz, 1H), 7.05-7.01 (m, 1H), 5.13 (d, J=4.5 Hz, 1H), 4.77-4.67 (m, 1H), 3.38 (s, 3H), 3.06-2.82 (m, 2H), 2.15-2.08 (m, 2H), 1.69 (d, J=6.9 Hz, 3H), 1.38 (s, 3H).

### Example 132: N-(3-(hydroxymethyl)-5-(1-(4-methyl-4H-1,2,4-triazol-3-ylthio)ethyl)phenyl)quinoline-2-carboxamide (132)

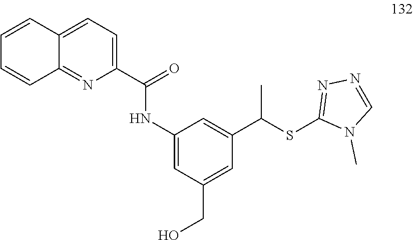 [SMILES: CC(Sc1nncn1C)c1cc(CO)cc(NC(=O)c2ccc3ccccc3n2)c1]

Compound 132 (200 mg, 64%) was obtained according to procedures disclosed herein as a yellow solid. MS (ESI) calculated for (C₂₂H₂₁N₅O₂S) [M+H]⁺, 420.1; found, 420.0. ¹H NMR (300 MHz, DMSO-d₆) δ 10.75 (s, 1H), 8.65 (d, J=8.4 Hz, 1H), 8.56 (s, 1H), 8.29-8.24 (m, 2H), 8.14 (d, J=7.8 Hz, 1H), 7.96-7.90 (m, 2H), 7.85 (s, 1H), 7.80-7.5 (m, 1H), 7.04 (s, 1H), 5.31-5.28 (m, 1H), 4.73-4.66 (m, 1H), 4.51 (d, J=5.7 Hz, 2H), 3.43 (s, 3H), 1.68 (d, J=6.9 Hz, 3H).

### Example 133: N-[3-[2-methoxy-1-[(4-methyl-1,2,4-triazol-3-yl)sulfanyl]ethyl]phenyl]-6-(trifluoromethyl)pyridine-2-carboxamide (133)

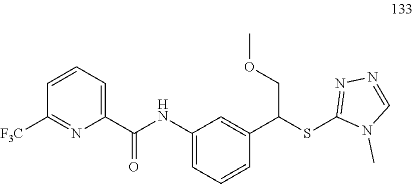 [SMILES: COCC(Sc1nncn1C)c1cccc(NC(=O)c2cccc(C(F)(F)F)n2)c1]

Compound 133 was obtained according to procedures disclosed herein in 49% yield. ¹H NMR (500 MHz, Methanol-d₄) δ 10.23 (s, 1H), 8.58 (s, 1H), 8.45 (d, J=7.8 Hz, 1H), 8.33-8.25 (m, 1H), 8.05 (dd, J=7.9, 1.0 Hz, 1H), 7.81 (dt, J=3.1, 1.9 Hz, 1H), 7.70 (dtd, J=8.1, 2.2, 1.0 Hz, 1H), 7.33 (t, J=7.9 Hz, 1H), 7.11 (dt, J=7.7, 1.3 Hz, 1H), 4.78 (t, J=6.7 Hz, 1H), 3.99-3.89 (m, 2H), 3.52 (s, 3H), 3.36 (s, 3H). LCMS: C₁₉H₁₈F₃N₅O₂S requires: 437.1, found: m/z 438.2 (M+H).

### Example 134: N-[3-[1-[(4-methyl-1,2,4-triazol-3-yl)sulfanyl]but-3-enyl]phenyl]quinoline-2-carboxamide (134)

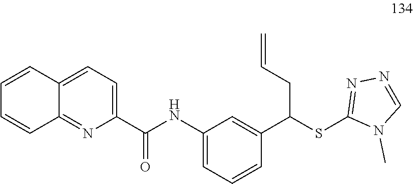 [SMILES: C=CCC(Sc1nncn1C)c1cccc(NC(=O)c2ccc3ccccc3n2)c1]

Compound 134 was obtained according to procedures disclosed herein in 35% yield. ¹H NMR (500 MHz, Methanol-d₄) δ 8.71 (s, 1H), 8.52 (dd, J=8.5, 0.9 Hz, 1H), 8.32-8.23 (m, 2H), 8.03 (dd, J=8.3, 1.4 Hz, 1H), 7.90-7.83 (m, 2H), 7.76-7.68 (m, 2H), 7.33 (t, J=7.9 Hz, 1H), 7.08-7.02 (m, 1H), 5.80 (ddt, J=17.1, 10.2, 6.9 Hz, 1H), 5.17 (dd, J=17.1, 1.6 Hz, 1H), 5.10-5.04 (m, 1H), 4.65 (t, J=7.7 Hz, 1H), 3.47 (s, 3H), 2.95-2.88 (m, 2H). LCMS: C₂₃H₂₁N₅OS requires: 415.2, found: m/z 416.3 (M+H).

### Example 135: N-[3-[1-[(4-methyl-1,2,4-triazol-3-yl)sulfanyl]but-3-enyl]phenyl]isoquinoline-3-carboxamide (135)

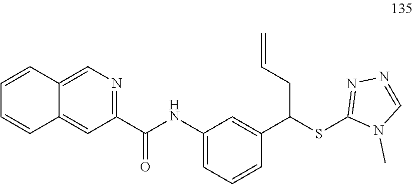 [SMILES: C=CCC(Sc1nncn1C)c1cccc(NC(=O)c2cc3ccccc3cn2)c1]

Compound 135 was obtained according to procedures disclosed herein in 24% yield. ¹H NMR (500 MHz, Methanol-d4) δ 9.36 (s, 1H), 8.70 (s, 1H), 8.66 (s, 1H), 8.22 (dd, J=8.1, 1.1 Hz, 1H), 8.13 (d, J=8.0 Hz, 1H), 7.96-7.77 (m, 4H), 7.69 (ddd, J=8.1, 2.2, 0.9 Hz, 1H), 7.32 (t, J=7.9 Hz, 1H), 7.04 (dt, J=7.7, 1.3 Hz, 1H), 5.80 (ddt, J=17.1, 10.2, 6.9 Hz, 1H), 5.17 (dd, J=17.1, 1.7 Hz, 1H), 5.07 (dd, J=10.2, 1.7 Hz, 1H), 4.65 (t, J=7.6 Hz, 1H), 3.47 (s, 3H), 2.94-2.86 (m, 2H). LCMS: C₂₃H₂₁N₅OS requires: 415.15, found: m/z 416 (M+H).

### Example 136: N-[3-[1-[(4-methyl-1,2,4-triazol-3-yl)sulfanyl]propyl]phenyl]quinoline-2-carboxamide (136)

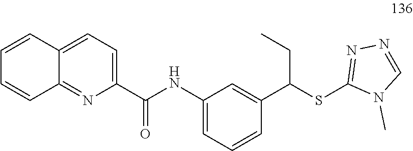 [SMILES: CCC(Sc1nncn1C)c1cccc(NC(=O)c2ccc3ccccc3n2)c1]

Compound 136 was obtained according to procedures disclosed herein in 60% yield. ¹H NMR (500 MHz, Methanol-d4) δ 8.88 (s, 1H), 8.51 (d, J=8.5 Hz, 1H), 8.28 (d, J=8.5 Hz, 1H), 8.25 (d, J=8.5 Hz, 1H), 8.01 (dd, J=8.3, 1.3 Hz, 1H), 7.90-7.82 (m, 2H), 7.75-1 FI (m, 2H), 7.34 (t, J=7.9 Hz, 1H), 7.06 (dt, J=7.8, 1.1 Hz, 1H), 4.53 (dd, J=8.7, 6.7 Hz, 1H), 3.50 (s, 3H), 2.26-2.11 (m, 2H), 1.04 (t, J=7.3 Hz, 3H). LCMS: C₂₂H₂₁N₅OS requires: 403.2, found: m/z 404.3 (M+H).

### Example 137: N-[3-[1-[(4-methyl-1,2,4-triazol-3-yl)sulfanyl]propyl]phenyl]isoquinoline-3-carboxamide (137)

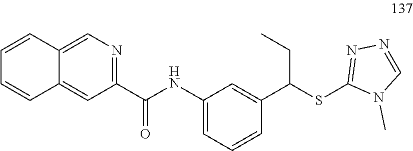 [SMILES: CCC(Sc1nncn1C)c1cccc(NC(=O)c2cc3ccccc3cn2)c1]

Compound 137 was obtained according to procedures disclosed herein in 63% yield. ¹H NMR (500 MHz, Methanol-d4) δ 9.36 (s, 1H), 8.91 (s, 1H), 8.66 (s, 1H), 8.21 (dd, J=8.2, 1.1 Hz, 1H), 8.12 (d, J=8.2 Hz, 1H), 7.89 (ddd, J=8.2, 6.9, 1.2 Hz, 1H), 7.86-7.79 (m, 2H), 7.68 (ddd, J=8.0, 2.0, 0.9 Hz, 1H), 7.33 (t, J=7.9 Hz, 1H), 7.09-7.03 (m, 1H), 4.54 (dd, J=8.6, 6.6 Hz, 1H), 3.51 (s, 3H), 2.26-2.10 (m, 2H), 1.04 (t, J=7.3 Hz, 3H). LCMS: C₂₂H₂₁N₅OS requires: 403.2, found: m/z 404.3 (M+H).

### Example 138: (S)-N-(3-(1-((4-methyl-4H-1,2,4-triazol-3-yl)thio)ethyl)phenyl)-4-(pyrrolidine-1-carbonyl)picolinamide (138)

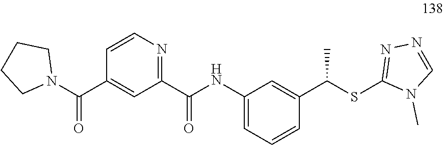 [SMILES: C[C@H](Sc1nncn1C)c1cccc(NC(=O)c2cc(C(=O)N3CCCC3)ccn2)c1]

Step 1: Synthesis of tert-butyl (S)-2-((3-(1-((4-methyl-4H-1,2,4-triazol-3-yl)thio)ethyl)phenyl)carbamoyl)isonicotinate. Followed General procedure 2 with addition of reagents at 0° C. under nitrogen to afford the title compound (1.2 g, 64%) as a colorless oil.

Step 2: Synthesis of (S)-2-((3-(1-((4-methyl-4H-1,2,4-triazol-3-yl)thio)ethyl)phenyl)carbamoyl)isonicotinic acid. To a solution of (S)-2-((3-(1-((4-methyl-4H-1,2,4-triazol-3-yl)thio)ethyl)phenyl)carbamoyl)isonicotinate (1.2 g, 2.73 mmol) in trifluoroacetic acid (3 mL) and dichloromethane (6 mL). The mixture was stirred at RT for 2 h, then concentrated to afford the title compound (1.3 g, crude) as a yellow solid, which was used without purification.

Step 3 (General procedure 5): Synthesis of 138. To a mixture of (S)-2-((3-(1-((4-methyl-4H-1,2,4-triazol-3-yl)thio)ethyl)phenyl)carbamoyl)isonicotinic acid (60 mg, 0.15 mmol), pyrrolidine (14 mg, 0.20 mmol), HATU (114 mg, 0.23 mmol) and N,N-diisopropylethylamine (60 mg, 0.45 mmol) in N,N-dimethylformamide (3 mL) was stirred at 25° C. for 3 h. The mixture was diluted by the addition of water (20 mL). General Work-up Procedure 1 was followed. The residue was purified by reverse phase flash column chromatography to afford 138 (26.7 mg, 38%) as a colorless solid. MS (ESI) calculated for (C₂₂H₂₄N₆O₂S) [M+H]⁺, 437.2; found, 437.2. ¹H NMR (300 MHz, DMSO-d₆) δ 10.68 (s, 1H), 8.83-8.81 (m, 1H), 8.52 (s, 1H), 8.16-8.15 (m, 1H), 7.94 (t, J=1.8 Hz, 1H), 7.84-7.79 (m, 1H), 7.77-7.73 (m, 1H), 7.28 (t, J=7.8 Hz, 1H), 7.05-7.02 (m, 1H), 4.66 (q, J=6.9 Hz, 1H), 3.49 (t, J=6.6 Hz, 2H), 3.43-3.41 (m, 2H), 3.41 (s, 3H), 1.89-1.85 (m, 4H), 1.64 (d, J=6.9 Hz, 3H).

### Example 139: 4-((R)-3-hydroxypyrrolidine-1-carbonyl)-N-(3-((S)-1-((4-methyl-4H-1,2,4-triazol-3-yl)thio)ethyl)phenyl)picolinamide (139)

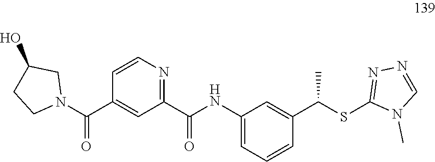 [SMILES: C[C@H](Sc1nncn1C)c1cccc(NC(=O)c2cc(C(=O)N3CC[C@@H](O)C3)ccn2)c1]

Followed General procedure 5 to afford 139 (5.8 mg, 8%) as an off-white solid. MS (ESI) calc'd for (C₂₂H₂₄N₆O₃S) [M+H]⁺, 453.2; found, 453.1. ¹H NMR (400 MHz, DMSO-d₆) δ 10.72 (s, 1H), 8.84 (d, J=4.8 Hz, 1H), 8.54 (s, 1H), 8.17-8.16 (m, 1H), 7.97 (s, 1H), 7.83 (d, J=8.0 Hz, 1H), 7.80-7.76 (m, 1H), 7.30 (t, J=8.0 Hz, 1H), 7.04 (d, J=8.0 Hz, 1H), 4.67 (q, J=6.8 Hz, 1H), 4.36-4.27 (m, 1H), 3.63-3.54 (m, 3H), 3.46-3.39 (m, 4H), 3.18-3.15 (m, 1H), 2.34-1.98 (m, 2H), 1.67 (d, J=7.2 Hz, 3H).

### Example 140: 4-[[(3S)-3-hydroxypyrrolidin-1-yl]carbonyl]-N-[3-[(1S)-1-[(4-methyl-4H-1,2,4-triazol-3-yl)sulfanyl]ethyl]phenyl]pyridine-2-carboxamide (140)

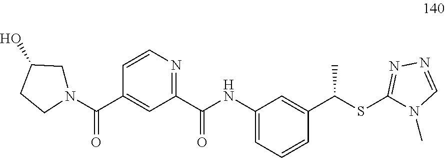 [SMILES: C[C@H](Sc1nncn1C)c1cccc(NC(=O)c2cc(C(=O)N3CC[C@H](O)C3)ccn2)c1]

Followed General procedure 5 to afford 140 (5.5 mg, 8%) as a light yellow solid. MS (ESI) calc'd for (C₂₂H₂₄N₆O₃S) [M+H]⁺, 453.2; found, 452.9. ¹H NMR (300 MHz, DMSO-d₆) δ 10.70 (s, 1H), 8.82 (m, 1H), 8.54 (s, 1H), 8.15-8.13 (m, 1H), 7.94 (t, J=1.8 Hz, 1H), 7.87-7.70 (m, 2H), 7.30 (t, J=7.8 Hz, 1H), 7.04 (d, J=7.8 Hz, 1H), 5.03 (m, 1H), 4.65 (q, J=6.9 Hz, 1H), 4.35-4.26 (m, 1H), 3.63-3.15 (m, 4H), 3.42 (s, 3H), 1.97-1.83 (m, 2H), 1.64 (d, J=6.9 Hz, 3H).

### Example 141: 4-((R)-3-methoxypyrrolidine-1-carbonyl)-N-(3-((S)-1-((4-methyl-4H-1,2,4-triazol-3-yl)thio)ethyl)phenyl)picolinamide (141)

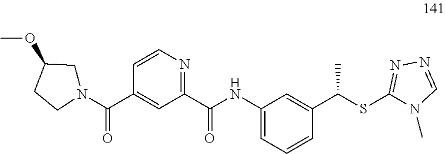 [SMILES: CO[C@@H]1CCN(C(=O)c2ccnc(C(=O)Nc3cccc([C@H](C)Sc4nncn4C)c3)c2)C1]

Followed General procedure 5 to afford 141 (19.9 mg, 33%) as a colorless solid. MS (ESI) calculated for (C₂₃H₂₆N₆O₃S) [M+H]⁺, 467.1; found, 467.2. ¹H NMR (400 MHz, DMSO-d₆) δ 10.70 (s, 1H), 8.84-8.83 (m, 1H), 8.53 (s, 1H), 8.16-8.14 (m, 1H), 7.96 (s, 1H), 7.87-7.66 (m, 2H), 7.30 (t, J=7.8 Hz, 1H), 7.05-7.03 (m, 1H), 4.67 (q, J=7.2 Hz, 1H), 4.13-3.84 (m, 1H), 3.66-3.57 (m, 2H), 3.56-3.39 (m, 2H), 3.35 (s, 3H), 3.18 (s, 3H), 2.16-1.90 (m, 2H), 1.66 (d, J=7.2 Hz, 3H).

### Example 142: N-(3-((S)-1-((4-methyl-4H-1,2,4-triazol-3-yl)thio)ethyl)phenyl)-4-((R)-2-methylpyrrolidine-1-carbonyl)picolinamide (142)

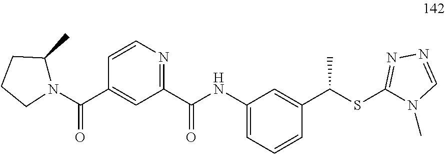 [SMILES: C[C@H](Sc1nncn1C)c1cccc(NC(=O)c2cc(C(=O)N3CCC[C@H]3C)ccn2)c1]

Followed General procedure 5 to afford 142 (12.0 mg, 10%) as a colorless solid. 1H NMR (300 MHz, DMSO-d₆) δ 10.71 (s, 1H), 8.83-8.81 (m, 1H), 8.54 (s, 1H), 8.13 (br, 1H), 7.97 (br, 1H), 7.80-7.78 (m, 2H), 7.30 (t, J=7.8 Hz, 1H), 7.04 (d, J=7.8 Hz, 1H), 4.67 (q, J=6.9 Hz, 1H), 4.20-4.18 (m, 1H), 3.57-3.38 (m, 5H), 2.11-2.08 (m, 1H), 2.01-1.82 (m, 1H), 1.80-1.52 (m, 5H), 1.32-1.29 (m, 2H), 0.87 (d, J=6.3 Hz, 1H). MS (ESI) calculated for (C₂₃H₂₆N₆O₂S) [M+H]⁺, 451.2; found, 451.2.

### Example 143: (S)-4-(azetidine-1-carbonyl)-N-(3-(1-((4-methyl-4H-1,2,4-triazol-3-yl)thio)ethyl)phenyl)picolinamide (143)

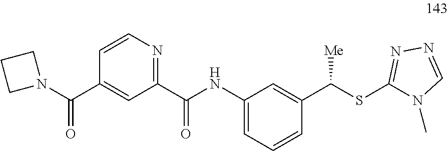 [SMILES: C[C@H](Sc1nncn1C)c1cccc(NC(=O)c2cc(C(=O)N3CCC3)ccn2)c1]

Followed General procedure 5 to afford 143 (13.6 mg, 25%) as a colorless solid. MS (ESI) calculated for (C₂₁H₂₂N₆O₂S) [M+H]⁺, 423.1; found, 423.1. ¹H NMR (300 MHz, DMSO-d₆) δ 10.69 (s, 1H), 8.84-8.82 (m, 1H), 8.52 (s, 1H), 8.23-8.21 (m, 1H), 7.95 (t, J=1.8 Hz, 1H), 7.89-7.75 (m, 2H), 7.28 (t, J=7.9 Hz, 1H), 7.07-6.98 (m, 1H), 4.65 (q, J=6.9 Hz, 1H), 4.34 (t, J=7.8 Hz, 2H), 4.09 (t, J=7.8 Hz, 2H), 3.37 (s, 3H), 2.28-2.26 (m, 2H), 1.64 (d, J=7.2 Hz, 3H).

### Example 144: (S)-N-(3-(1-((4-methyl-4H-1,2,4-triazol-3-yl)thio)ethyl)phenyl)-4-(piperidine-1-carbonyl)picolinamide (144)

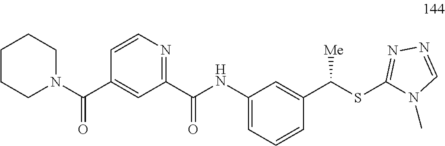 [SMILES: C[C@H](Sc1nncn1C)c1cccc(NC(=O)c2cc(C(=O)N3CCCCC3)ccn2)c1]

Followed General procedure 5 to afford 144 (26.3 mg, 45%) as a colorless solid. MS (ESI) calculated for (C₂₃H₂₆N₆O₂S) [M+H]⁺, 451.2; found, 451.4. ¹H NMR (300 MHz, DMSO-d₆) δ 10.71 (s, 1H), 8.83-8.81 (m, 1H), 8.54 (s, 1H), 8.05-8.04 (m, 1H), 7.97 (t, J=1.8 Hz, 1H), 7.88-7.78 (m, 1H), 7.68-7.66 (m, 1H), 7.30 (t, J=7.8 Hz, 1H), 7.04 (d, J=7.8 Hz, 1H), 4.67 (q, J=7.0 Hz, 1H), 3.63-3.61 (m, 2H), 3.39 (s, 3H), 3.24-3.22 (m, 2H), 1.68-1.48 (m, 6H), 1.64 (d, J=7.2 Hz, 3H).

### Example 145: (S)-4-(4-hydroxypiperidine-1-carbonyl)-N-(3-(1-((4-methyl-4H-1,2,4-triazol-3-yl)thio)ethyl)phenyl)picolinamide (145)

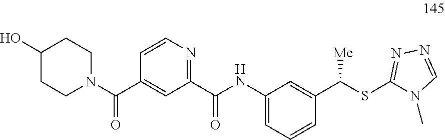 [SMILES: C[C@H](Sc1nncn1C)c1cccc(NC(=O)c2cc(C(=O)N3CCC(O)CC3)ccn2)c1]

Followed General procedure 5 to afford 145 (27.2 mg, 37%) as a colorless solid. MS (ESI) calculated for (C₂₃H₂₆N₆O₃S) [M+H]⁺, 467.2; found, 467.4. ¹H NMR (400 MHz, DMSO-d₆) δ 10.70 (s, 1H), 8.82-8.81 (m, 1H), 8.53 (s, 1H), 8.05-8.04 (m, 1H), 7.96 (s, 1H), 7.83 (d, J=8.0 Hz, 1H), 7.68-7.66 (m, 1H), 7.30 (t, J=7.6 Hz, 1H), 7.08-7.00 (m, 1H), 4.82 (s, 1H), 4.67 (q, J=6.8 Hz, 1H), 4.06-3.97 (m, 1H), 3.79-3.74 (m, 1H), 3.39 (s, 3H), 3.39-3.32 (m, 2H), 3.12-3.10 (m, 1H), 1.83-1.82 (m, 1H), 1.70-1.68 (m, 1H), 1.66 (d, J=7.2 Hz, 3H), 1.45-1.43 (m, 1H), 1.36-1.34 (m, 1H).

### Example 146: 4-((R)-3-aminopyrrolidine-1-carbonyl)-N-(3-((S)-1-((4-methyl-4H-1,2,4-triazol-3-yl)thio)ethyl)phenyl)picolinamide (146)

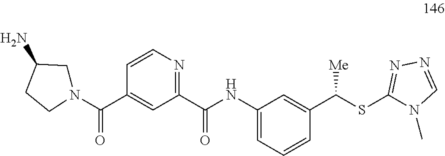 [SMILES: C[C@H](Sc1nncn1C)c1cccc(NC(=O)c2cc(C(=O)N3CC[C@@H](N)C3)ccn2)c1]

Compound 146 (16.9 mg, 49%) was obtained according to procedures disclosed herein as a yellow solid. MS (ESI) calculated for (C₂₂H₂₅N₇O₂S) [M+H]⁺, 452.2; found, 452.2. ¹H NMR (300 MHz, Methanol-d₄) δ 9.56 (s, 1H), 8.89-8.88 (m, 1H), 8.38-8.37 (m, 1H), 7.94 (s, 1H), 7.83-7.81 (m, 1H), 7.70-7.69 (m, 1H), 7.41 (t, J=7.8 Hz, 1H), 7.25 (d, J=7.8 Hz, 1H), 5.00 (q, J=6.9 Hz, 1H), 4.12-3.74 (m, 5H), 3.41 (s, 3H), 2.54-2.39 (m, 1H), 2.19-2.16 (m, 1H), 1.89 (d, J=6.9 Hz, 3H). c

### Example 147: 4-((S)-3-aminopyrrolidine-1-carbonyl)-N-(3-((S)-1-(4-methyl-4H-1,2,4-triazol-3-ylthio)ethyl)phenyl)picolinamide (147)

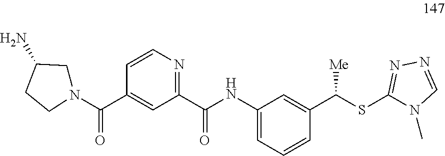 [SMILES: C[C@H](Sc1nncn1C)c1cccc(NC(=O)c2cc(C(=O)N3CC[C@H](N)C3)ccn2)c1]

Followed General procedure 5 to afford 147 (15.2 mg, 53%) as a colorless solid. MS (ESI) calculated for (C₂₂H₂₅N₇O₂S) [M+H]⁺, 452; found, 452. ¹H NMR (300 MHz, Methanol-d₄) δ 9.56 (s, 1H), 8.89 (s, 1H), 8.39-8.36 (m, 1H), 7.95 (s, 1H), 7.81 (s, 1H), 7.70 (d, J=8.1 Hz, 1H), 7.41 (t, J=7.8 Hz, 1H), 7.25 (d, J=7.8 Hz, 1H), 5.00-4.90 (m, 1H), 4.08-3.76 (m, 3H), 3.71 (s, 3H), 3.70-3.68 (m, 1H), 3.60-3.56 (m, 1H), 2.47-2.45 (m, 1H), 2.20-2.19 (m, 1H), 1.89 (d, J=6.9 Hz, 3H).

### Example 148: (S)-4-(3-hydroxyazetidine-1-carbonyl)-N-(3-(1-((4-methyl-4H-1,2,4-triazol-3-yl)thio)ethyl)phenyl)picolinamide (148)

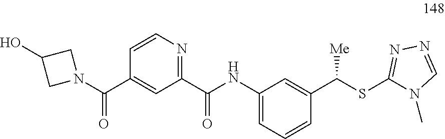 [SMILES: C[C@H](Sc1nncn1C)c1cccc(NC(=O)c2cc(C(=O)N3CC(O)C3)ccn2)c1]

Followed General procedure 5 to afford 148 (14 mg, 12%) as a light yellow solid. MS (ESI) calc'd for (C₂₁H₂₂N₆O₃S) [M+H]⁺, 439; found, 439. ¹H NMR (300 MHz, DMSO-d₆) δ 10.72 (s, 1H), 8.86 (d, J=5.1 Hz, 1H), 8.55 (s, 1H), 8.24 (s, 1H), 7.98 (d, J=2.1 Hz, 1H), 7.89-7.80 (m, 2H), 7.31 (t, J=7.8 Hz, 1H), 7.05 (d, J=7.2 Hz, 1H), 5.85 (d, J=6.0 Hz, 1H), 4.68 (q, J=6.9 Hz, 1H), 4.56-4.47 (m, 2H), 4.39-4.27 (m, 1H), 4.11-4.10 (m, 1H), 3.87-3.83 (m, 1H), 3.40 (s, 3H), 1.68 (d, J=6.9 Hz, 3H).

### Example 149: 4-((R)-3-hydroxypiperidine-1-carbonyl)-N-(3-((S)-1-((4-methyl-4H-1,2,4-triazol-3-yl)thio)ethyl)phenyl)picolinamide (149)

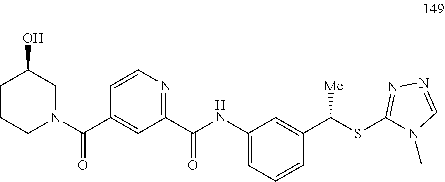 [SMILES: C[C@H](Sc1nncn1C)c1cccc(NC(=O)c2cc(C(=O)N3CCC[C@@H](O)C3)ccn2)c1]

Followed General procedure 5 to afford 149 (23 mg, 19%) as a light yellow solid. MS (ESI) calc'd for (C₂₃H₂₆N₆O₃S) [M+H]⁺, 467; found, 467. ¹H NMR (300 MHz, DMSO-d₆) δ 10.72 (s, 1H), 8.83 (d, J=4.8 Hz, 1H), 8.55 (s, 1H), 8.10-8.06 (m, 1H), 7.97 (d, J=2.1 Hz, 1H), 7.84 (d, J=7.8 Hz, 1H), 7.69-7.66 (m, 1H), 7.31 (t, J=7.8 Hz, 1H), 7.05 (d, J=7.8 Hz, 1H), 5.06-4.86 (m, 1H), 4.68 (q, J=6.9 Hz, 1H), 3.61-3.51 (m, 3H), 3.40 (s, 3H), 3.26-2.94 (m, 2H), 1.87-1.80 (m, 2H), 1.68 (d, J=6.9 Hz, 3H), 1.59-1.25 (m, 2H).

### Example 150: 4-((S)-3-hydroxypiperidine-1-carbonyl)-N-(3-((S)-1-((4-methyl-4H-1,2,4-triazol-3-yl)thio)ethyl)phenyl)picolinamide (150)

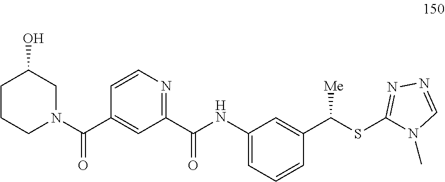 [SMILES: C[C@H](Sc1nncn1C)c1cccc(NC(=O)c2cc(C(=O)N3CCC[C@H](O)C3)ccn2)c1]

Followed General procedure 5 to afford 150 (44 mg, 36%) as a colorless solid. MS (ESI) calc'd for (C₂₃H₂₆N₆O₃S) [M+H]⁺, 467; found, 467. ¹H NMR (300 MHz, DMSO-d₆) δ 10.71 (s, 1H), 8.82 (dd, J=4.8, 0.6 Hz, 1H), 8.54 (s, 1H), 8.09-8.05 (m, 1H), 7.97 (s, 1H), 7.84 (d, J=8.4 Hz, 1H), 7.69-7.67 (m, 1H), 7.30 (t, J=7.8 Hz, 1H), 7.05 (d, J=7.8 Hz, 1H), 4.86-4.66 (m, 1H), 4.67 (q, J=6.9 Hz, 1H), 3.70-3.50 (m, 3H), 3.39 (s, 3H), 3.25-2.76 (m, 2H), 1.90-1.80 (m, 2H), 1.67 (d, J=6.9 Hz, 3H), 1.55-1.40 (m, 2H).

### Example 151: 4-((2S,4R)-4-hydroxy-2-methylpyrrolidine-1-carbonyl)-N-(3-((S)-1-((4-methyl-4H-1,2,4-triazol-3-yl)thio)ethyl)phenyl)picolinamide (151)

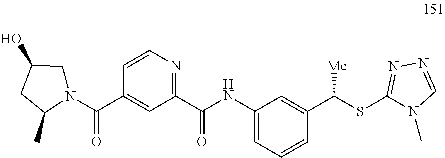 [SMILES: C[C@H](Sc1nncn1C)c1cccc(NC(=O)c2cc(C(=O)N3C[C@H](O)C[C@@H]3C)ccn2)c1]

Followed General procedure 5 to afford 151 (20 mg, 27%) as a light yellow solid. MS (ESI) calc'd for (C₂₃H₂₆N₆O₃S) [M+H]⁺, 467; found, 467. ¹H NMR (300 MHz, DMSO-d₆) δ 10.72 (s, 1H), 8.83 (d, J=4.8 Hz, 1H), 8.54 (s, 1H), 8.13 (s, 1H), 7.97 (s, 1H), 7.83 (d, J=7.8 Hz, 1H), 7.77-7.73 (m, 1H), 7.30 (t, J=7.8 Hz, 1H), 7.04 (d, J=7.8 Hz, 1H), 5.13 (d, J=3.6 Hz, 1H), 4.65 (q, J=6.9 Hz, 1H), 4.37-3.92 (m, 2H), 3.49-3.43 (m, 1H), 3.39-3.26 (m, 4H), 2.32-2.23 (m, 1H), 1.67 (d, J=7.2 Hz, 3H), 1.61-1.55 (m, 1H), 1.41 (d, J=6.3 Hz, 2H), 1.01 (d, J=6.3 Hz, 1H).

### Example 152: 4-((2R,4S)-4-hydroxy-2-methylpyrrolidine-1-carbonyl)-N-(3-((S)-1-((4-methyl-4H-1,2,4-triazol-3-yl)thio)ethyl)phenyl)picolinamide (152)

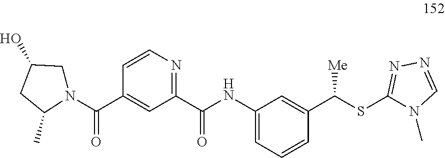 [SMILES: C[C@H](Sc1nncn1C)c1cccc(NC(=O)c2cc(C(=O)N3C[C@@H](O)C[C@H]3C)ccn2)c1]

Followed General procedure 5 to afford 152 (25 mg, 34%) as a light yellow solid. MS (ESI) calc'd for (C₂₃H₂₆N₆O₃S) [M+H]⁺, 467; found, 467. ¹H NMR (300 MHz, DMSO-d₆) δ 10.71 (s, 1H), 8.83 (d, J=4.8 Hz, 1H), 8.54 (s, 1H), 8.13 (s, 1H), 7.96 (s, 1H), 7.82 (d, J=7.5 Hz, 1H), 7.76-7.74 (m, 1H), 7.33-7.28 (m, 1H), 7.04 (d, J=7.5 Hz, 1H), 5.13 (d, J=3.6 Hz, 1H), 4.67 (q, J=6.9 Hz, 1H), 4.39-3.93 (m, 2H), 3.82-3.52 (m, 1H), 3.43-3.32 (m, 4H), 2.31-2.19 (m, 1H), 1.67 (d, J=6.9 Hz, 3H), 1.61-1.55 (m, 1H), 1.41 (d, J=6.3 Hz, 2H), 1.01 (d, J=6.3 Hz, 1H).

### Example 153: 4-((2R,4R)-4-hydroxy-2-methylpyrrolidine-1-carbonyl)-N-(3-((S)-1-((4-methyl-4H-1,2,4-triazol-3-yl)thio)ethyl)phenyl)picolinamide (153)

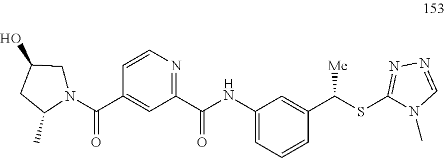 [SMILES: C[C@H](Sc1nncn1C)c1cccc(NC(=O)c2cc(C(=O)N3C[C@H](O)C[C@H]3C)ccn2)c1]

Followed General procedure 5 to afford 153 (285 mg, 38%) as a light yellow solid. MS (ESI) calc'd for (C₂₃H₂₆N₆O₃S) [M+H]⁺, 467.2; found, 467.2. ¹H NMR (300 MHz, DMSO-d₆) δ 10.72 (s, 1H), 8.85-8.83 (m, 1H), 8.54 (s, 1H), 8.14 (s, 1H), 7.96 (s, 1H), 7.85-7.82 (m, 1H), 7.76-7.74 (m, 1H), 7.33-7.28 (m, 1H), 7.04 (d, J=7.8 Hz, 1H), 5.15-4.90 (m, 1H), 4.67 (q, J=6.9 Hz, 1H), 4.28-4.05 (m, 2H), 3.69-3.64 (m, 1H), 3.35 (s, 3H), 3.14-3.10 (m, 1H), 2.12-2.05 (m, 1H), 1.73-1.65 (m, 4H), 1.33 (d, J=6.9 Hz, 3H).

### Example 154: 4-((2S,4S)-4-hydroxy-2-methylpyrrolidine-1-carbonyl)-N-(3-((S)-1-((4-methyl-4H-1,2,4-triazol-3-yl)thio)ethyl)phenyl)picolinamide (154)

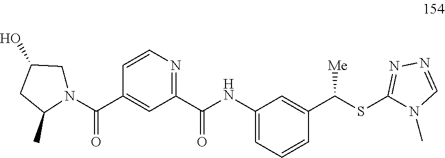 [SMILES: C[C@H](Sc1nncn1C)c1cccc(NC(=O)c2cc(C(=O)N3C[C@@H](O)C[C@@H]3C)ccn2)c1]

Followed General procedure 5 to afford 154 (26 mg, 35%) as a light yellow solid. MS (ESI) calc'd for (C₂₃H₂₆N₆O₃S) [M+H]⁺, 467; found, 467. ¹H NMR (300 MHz, DMSO-d₆) δ 10.72 (s, 1H), 8.85-8.83 (m, 1H), 8.54 (s, 1H), 8.14 (s, 1H), 7.96 (s, 1H), 7.85-7.82 (m, 1H), 7.76-7.74 (m, 1H), 7.33-7.28 (m, 1H), 7.04 (d, J=7.5 Hz, 1H), 5.15-4.91 (m, 1H), 4.67 (q, J=6.9 Hz, 1H), 4.30-4.03 (m, 2H), 3.69-3.51 (m, 1H), 3.36 (s, 3H), 3.14-3.10 (m, 1H), 2.12-2.05 (m, 1H), 1.73-1.67 (m, 4H), 1.33 (d, J=6.9 Hz, 3H).

### Example 155: 4-[[(3S,4S)-3,4-dihydroxypyrrolidin-1-yl]carbonyl]-N-[3-[(1S)-1-[(4-methyl-4H-1,2,4-triazol-3-yl)sulfanyl]ethyl]phenyl]pyridine-2-carboxamide (155)

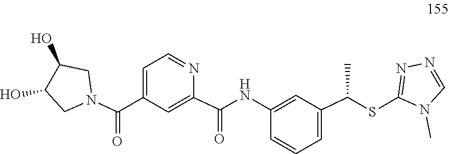 [SMILES: C[C@H](Sc1nncn1C)c1cccc(NC(=O)c2cc(C(=O)N3C[C@H](O)[C@@H](O)C3)ccn2)c1]

Following General procedure 5 to afford 155 (59 mg, 80%) as a colorless solid. 1H NMR (300 MHz, DMSO-d₆+D₂O) δ 8.97-8.62 (m, 2H), 8.19-8.16 (m, 1H), 7.92 (br, 1H), 7.84-7.82 (m, 2H), 7.30 (t, J=7.8 Hz, 1H), 7.04 (d, J=7.2 Hz, 1H), 4.68 (br, 1H), 4.02-3.94 (m, 2H), 3.72-3.66 (m, 2H), 3.44-3.42 (m, 1H), 3.39 (s, 3H), 3.17-3.14 (m, 1H), 1.66 (d, J=6.6 Hz, 3H). MS (ESI) calc'd for (C₂₂H₂₄N₆O₄S) [M+H]⁺, 469; found, 469.

### Example 156: N⁴-((R)-2-hydroxypropyl)-N²-(3-((S)-1-((4-methyl-4H-1,2,4-triazol-3-yl)thio)ethyl)phenyl)pyridine-2,4-dicarboxamide (156)

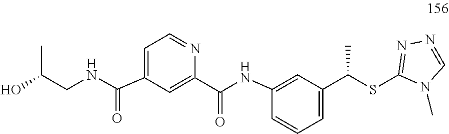 [SMILES: C[C@H](Sc1nncn1C)c1cccc(NC(=O)c2cc(C(=O)NC[C@@H](C)O)ccn2)c1]

Followed General procedure 5 to afford 156 (52 mg, 75%) as a colorless solid. MS (ESI) calc'd for (C₂₁H₂₄N₆O₃S) [M+H]⁺, 441; found, 441. ¹H NMR (300 MHz, DMSO-d₆), δ 10.72 (s, 1H), 8.99 (t, J=5.7 Hz, 1H), 8.88 (d, J=5.4 Hz, 1H), 8.57-8.54 (m, 2H), 8.05-8.03 (m, 1H), 7.96 (s, 1H), 7.87-7.84 (m, 1H), 7.30 (t, J=7.8 Hz, 1H), 7.04 (d, J=8.1 Hz, 1H), 4.82 (d, J=4.8 Hz, 1H), 4.66 (q, J=6.6 Hz, 1H), 3.87-3.79 (m, 1H), 3.39 (s, 3H), 3.27-3.22 (m, 2H), 1.66 (d, J=6.9 Hz, 3H), 1.09 (d, J=6.3 Hz, 3H).

### Example 157: N⁴-((S)-2-hydroxypropyl)-N²-(3-((S)-1-((4-methyl-4H-1,2,4-triazol-3-yl)thio)ethyl)phenyl)pyridine-2,4-dicarboxamide (157)

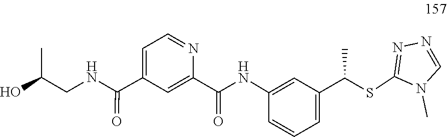 [SMILES: C[C@H](O)CNC(=O)c1ccnc(C(=O)Nc2cccc([C@H](C)Sc3nncn3C)c2)c1]

Followed General procedure 5 to afford 157 (29 mg, 41%) as a colorless solid. MS (ESI) calc'd for (C₂₁H₂₄N₆O₃S) [M+H]⁺, 441; found, 441. ¹H NMR (300 MHz, DMSO-d₆), δ 10.72 (s, 1H), 8.99 (t, J=5.7 Hz, 1H), 8.88-8.70 (m, 1H), 8.56-8.55 (m, 2H), 8.05-8.03 (m, 1H), 7.96 (s, 1H), 7.84 (d, J=8.1 Hz, 1H), 7.30 (t, J=7.8 Hz, 1H), 7.04 (d, J=7.8 Hz, 1H), 4.66 (q, J=6.9 Hz, 1H), 3.88-3.78 (m, 1H), 3.39 (s, 3H), 3.27-3.22 (m, 2H), 1.66 (d, J=6.9 Hz, 3H), 1.09 (d, J=6.3 Hz, 3H).

### Example 158: N-(3-((A)-1-((4-methyl-4H-1,2,4-triazol-3-yl)thio)ethyl)phenyl)-4-(3-(methylsulfonyl)pyrrolidine-1-carbonyl)picolinamide (158)

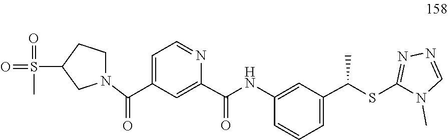 [SMILES: C[C@H](Sc1nncn1C)c1cccc(NC(=O)c2cc(C(=O)N3CCC(S(C)(=O)=O)C3)ccn2)c1]

Followed General procedure 5 to afford 158 (27 mg, 41%) as a colorless solid. MS (ESI) calc'd for (C₂₃H₂₆N₆O₄S₂) [M+H]⁺, 515; found, 515. ¹H NMR (300 MHz, DMSO-d₆) δ 10.72 (s, 1H), 8.87-8.84 (m, 1H), 8.55 (s, 1H), 8.16 (d, J=7.8 Hz, 1H), 7.96 (s, 1H), 7.84-7.75 (m, 2H), 7.30 (t, J=7.8 Hz, 1H), 7.04 (d, J=7.5 Hz, 1H), 4.66 (q, J=6.9 Hz, 1H), 3.99-3.72 (m, 5H), 3.38 (s, 3H), 3.04 (s, 3H), 2.38-3.27 (m, 2H), 1.66 (d, J=6.9 Hz, 3H).

### Example 159: 4-((R)-3-(dimethylamino)pyrrolidine-1-carbonyl)-N-(3-((S)-1-((4-methyl-4H-1,2,4-triazol-3-yl)thio)ethyl)phenyl)picolinamide (159)

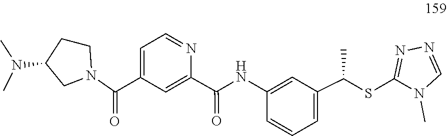 [SMILES: C[C@H](Sc1nncn1C)c1cccc(NC(=O)c2cc(C(=O)N3CC[C@@H](N(C)C)C3)ccn2)c1]

Followed General procedure 5 to afford 159 (20 mg, 28%) as a colorless solid. MS (ESI) calc'd for (C₂₄H₂₉N₇O₂S) [M+H]⁺, 480; found, 480. ¹H NMR (300 MHz, DMSO-d₆) δ 10.71 (s, 1H), 8.83 (d, J=2.1 Hz, 1H), 8.54 (s, 1H), 8.16 (s, 1H), 7.96 (s, 1H), 7.84-7.76 (m, 2H), 7.30 (t, J=7.8 Hz, 1H), 7.04 (d, J=7.8 Hz, 1H), 4.65 (q, J=7.2 Hz, 1H), 3.80-3.42 (m, 6H), 3.28-3.21 (m, 1H), 2.75-2.70 (m, 1H), 2.19 (s, 3H), 2.09-2.02 (m, 4H), 1.81-1.70 (m, 1H), 1.67 (d, J=7.2 Hz, 3H).

### Example 160: 4-((S)-3-(dimethylamino)pyrrolidine-1-carbonyl)-N-(3-((S)-1-((4-methyl-4H-1,2,4-triazol-3-yl)thio)ethyl)phenyl)picolinamide (160)

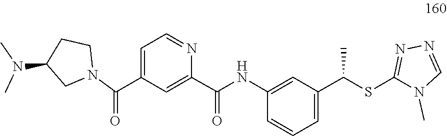 [SMILES: C[C@H](Sc1nncn1C)c1cccc(NC(=O)c2cc(C(=O)N3CC[C@H](N(C)C)C3)ccn2)c1]

Followed General procedure 5 to afford 160 (26 mg, 35%) as a colorless solid. MS (ESI) calc'd for (C₂₄H₂₉N₇O₂S) [M+H]⁺, 480; found, 480. ¹H NMR (300 MHz, DMSO-d₆) δ 10.71 (s, 1H), 8.83 (d, J=4.8 Hz, 1H), 8.54 (s, 1H), 8.16 (s, 1H), 7.96 (s, 1H), 7.84-7.76 (m, 2H), 7.30 (t, J=7.8 Hz, 1H), 7.04 (d, J=7.8 Hz, 1H), 4.68 (q, J=6.9 Hz, 1H), 3.80-3.43 (m, 6H), 3.28-3.21 (m, 1H), 2.75-2.70 (m, 1H), 2.19 (s, 3H), 2.09-2.00 (m, 4H), 1.81-1.70 (m, 1H), 1.66 (d, J=6.9 Hz, 3H).

### Example 161: 4-[[(3S)-3-hydroxypyrrolidin-1-yl]carbonyl]-N-[3-[(1R)-1-[(4-methyl-4H-1,2,4-triazol-3-yl)sulfanyl]ethyl]phenyl]pyridine-2-carboxamide (161)

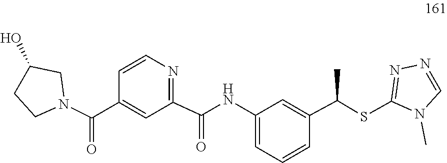 [SMILES: C[C@@H](Sc1nncn1C)c1cccc(NC(=O)c2cc(C(=O)N3CC[C@H](O)C3)ccn2)c1]

Compound 161 was obtained according to procedures disclosed herein as a light yellow solid (1.4 g, 19%). MS (ESI) calc'd for (C₂₂H₂₄N₆O₃S) [M+H]⁺, 453; found, 452. ¹H NMR (300 MHz, DMSO-d₆) δ 10.72 (s, 1H), 8.84 (m, 1H), 8.52 (s, 1H), 8.15 (d, J=0.6 Hz, 1H), 7.94 (d, J=1.8 Hz, 1H), 7.87-7.68 (m, 2H), 7.28 (t, J=7.8 Hz, 1H), 7.02 (dt, J=7.8, 1.2 Hz, 1H), 5.05 (br, 1H), 4.65 (q, J=6.9 Hz, 1H), 4.35-4.26 (m, 1H), 3.56-3.50 (m, 3H), 3.45-3.36 (m, 3.4H), 3.18-3.14 (m, 0.6H), 2.00-1.83 (m, 2H), 1.66 (d, J=6.9 Hz, 3H).

### Example 162: 2-[3-[1-[(4-methyl-1,2,4-triazol-3-yl)sulfanyl]ethyl]phenyl]isoindolin-1-one (162)

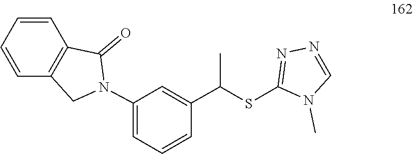 [SMILES: CC(Sc1nncn1C)c1cccc(N2Cc3ccccc3C2=O)c1]

Compound 162 was obtained according to procedures disclosed herein (28 mg, yield 44%). LCMS C₁₉H₁₈N₄OS requires: 350, found: m/z, 351 (M+H).

### Example 163: 6-chloro-2-[3-[1-[(4-methyl-1,2,4-triazol-3-yl)sulfanyl]ethyl]phenyl]isoindolin-1-one (163)

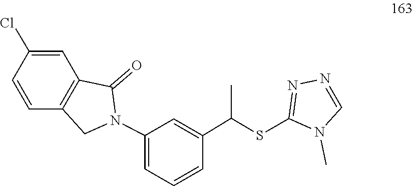 [SMILES: CC(Sc1nncn1C)c1cccc(N2Cc3ccc(Cl)cc3C2=O)c1]

Compound 163 was obtained according to procedures disclosed herein (8.5 mg, yield 19%). ¹H NMR (500 MHz, Chloroform-d) δ 8.42 (s, 1H), 7.87 (d, J=2.0 Hz, 1H), 7.80 (ddd, J=8.2, 2.3, 1.0 Hz, 1H), 7.65 (t, J=2.0 Hz, 1H), 7.58 (dd, J=8.1, 2.0 Hz, 1H), 7.48 (d, J=8.1 Hz, 1H), 7.37 (t, J=8.0 Hz, 1H), 7.15 (dt, J=7.8, 1.3 Hz, 1H), 4.87 (q, J=7.1 Hz, 1H), 4.79 (s, 2H), 3.41 (s, 3H), 1.84 (d, J=7.1 Hz, 3H). LCMS: C₁₉H₁₇ClN₄OS requires: 384, found: m/z 385 [M+H]⁺.

### Example 164: (S)-5-chloro-2-(3-(1-((4-methyl-4H-1,2,4-triazol-3-yl)thio)ethyl)phenyl)isoindolin-1-one (164)

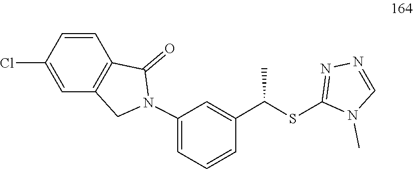 [SMILES: C[C@H](Sc1nncn1C)c1cccc(N2Cc3cc(Cl)ccc3C2=O)c1]

Step 1: Synthesis of 5-chloro-2-(3-(l-hydroxyethyl)phenyl)isoindolin-1-one. A suspension of copper (I) iodide (88 mg, 0.46 mmol), l-(3-bromophenyl)ethan-1-ol (1.85 g, 9.19 mmol) potassium carbonate (0.65 g, 4.59 mmol) and 5-chloroisoindolin-1-one (0.77 g, 4.59 mmol) in anhydrous N,N-dimethylformamide (3.8 mL) was purged with nitrogen and heated at 150° C. for 16 h. The mixture was diluted with 10% ammonium hydroxide solution. General Work-up Procedure 1 was followed. The residue was purified on silica gel (ethyl acetate in hexanes 40% isocratic) to give the title compound as an off-white solid. Yield: 0.42 g (32%).

Step 2:5-chloro-2-(3-(1-((4-methyl-4H-1,2,4-triazol-3-yl)thio)ethyl)phenyl)isoindolin-1-one. A solution of 3-mercapto-4-methyl-4H-1,2,4-triazole (0.082 g, 0.695 mmol), triphenylphosphine resin (1.5 mmol/g loading, 0.69 g, 1.04 mmol) and 5-chloro-2-(3-(l-hydroxyethyl)phenyl)isoindolin-1-one (0.10 g, 0.34 mmol) in THF (3.5 mL) was treated with diisopropyl azodicarboxylate (0.14 g, 0.69 mmol). The mixture was stirred at rt for 2 h. The mixture was filtered through Celite washing with EtOAc. The mixture was concentrated and purified on silica gel to afford the title compound (69 mg, 51%) as an off-white foam.

Step 3: (S)-5-chloro-2-(3-(1-((4-methyl-4H-1,2,4-triazol-3-yl)thio)ethyl)phenyl)isoindolin-1-one. 5-chloro-2-[3-[1-[(4-methyl-1,2,4-triazol-3-yl)sulfanyl]ethyl]phenyl]isoindolin-1-one (0.11 g, 0.28 mmol) was resolved using prep-chiral SFC followed by lyophilization in acetonitrile/water to provide the title compound (20 mg, 18%) as an off-white powder. ¹H NMR (500 MHz, DMSO-d₆) δ 8.55 (d, J=1.4 Hz, 1H), 7.86-7.76 (m, 4H), 7.62 (dd, J=8.1, 1.9 Hz, 1H), 7.39 (t, J=7.9 Hz, 1H), 7.13 (d, J=7.5 Hz, 1H), 5.01 (s, 1H), 4.72 (q, J=6.9 Hz, 1H), 3.38 (s, 3H), 1.69 (d, J=7.0 Hz, 3H). LCMS: C₁₉H₁₇ClN₄OS requires: 384, found: m/z=385 [M+H]+.

### Example 165: 6-(1-hydroxy-1-methyl-ethyl)-2-[3-[1-[(4-methyl-1,2,4-triazol-3-yl)sulfanyl]ethyl]phenyl]isoindolin-1-one (165)

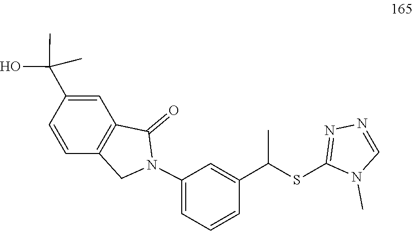 [SMILES: CC(Sc1nncn1C)c1cccc(N2Cc3ccc(C(C)(C)O)cc3C2=O)c1]

Compound 165 was obtained according to procedures disclosed herein (45 mg, 25%). ¹H NMR (500 MHz, Chloroform-d) δ 8.05 (s, 1H), 8.03-7.97 (m, 1H), 7.88 (ddd, J=8.4, 2.3, 1.0 Hz, 1H), 7.82 (dd, J=7.9, 1.7 Hz, 1H), 7.57 (t, J=2.0 Hz, 1H), 7.50 (dd, J=8.0, 0.9 Hz, 1H), 7.34 (t, J=7.9 Hz, 1H), 7.10 (dt, J=7.7, 1.3 Hz, 1H), 4.80 (d, J=7.2 Hz, 1H), 4.77 (d, J=5.1 Hz, 2H), 3.28 (s, 3H), 1.83 (d, J=7.1 Hz, 3H), 1.64 (s, 6H). LCMS: C₂₂H₂₄N₄O₂S requires: 408, found: m/z, 409 (M+H).

### Example 166: 2-(3-(1-((4-methyl-4H-1,2,4-triazol-3-yl)thio)ethyl)phenyl)-4-(trifluoromethyl)isoindolin-1-one (166)

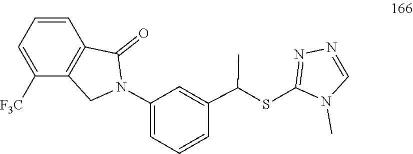 [SMILES: CC(Sc1nncn1C)c1cccc(N2Cc3c(cccc3C(F)(F)F)C2=O)c1]

Under nitrogen, copper(I) iodide (0.08 g, 0.41 mmol) was added to a N,N-dimethylformamide (2 mL) solution containing potassium carbonate (37 mg, 0.27 mmol), 4-(trifluoromethyl)isoindolin-1-one (0.04 g, 0.20 mmol) and 3-[1-(3-bromophenyl)ethylsulfanyl]-4-methyl-1,2,4-triazole (0.06 g, 0.20 mmol). The solution was then stirred at 150° C. for 16 h. The reaction was filtered and the solution was purified by HPLC to yield the final product (7 mg, yield 3.5%). ¹H NMR (500 MHz, Chloroform-d) δ 8.44 (s, 1H), 8.11 (d, J=7.7 Hz, 1H), 7.89-7.81 (m, 2H), 7.71-7.63 (m, 2H), 7.37 (t, J=7.9 Hz, 1H), 7.14 (d, J=7.6 Hz, 1H), 5.11-4.90 (m, 2H), 3.41 (s, 3H), 1.84 (d, J=6.8 Hz, 3H). LCMS: C₂₀H₁₇F₃N₄OS requires: 418, found: m/z, 419 (M+H).

### (S)-2-(3-(1-((4-methyl-4H-1,2,4-triazol-3-yl)thio)ethyl)phenyl)-4-(trifluoromethyl)isoindolin-1-one (166a)

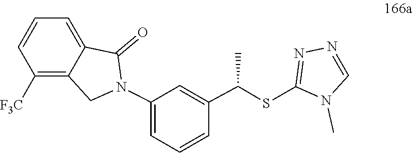 [SMILES: C[C@H](Sc1nncn1C)c1cccc(N2Cc3c(cccc3C(F)(F)F)C2=O)c1]

To a solution of methyl 2-(bromomethyl)-3-(trifluoromethyl)benzoate (200 mg, 0.67 mmol), A-a (157 mg, 0.67 mmol) in ethanol (2 mL) was added triethylamine (0.14 mL, 1.0 mmol). The solution was stirred at 80° C. overnight, and then concentrated, The residue was puritify by HPLC to afford 166a (80 mg, 28%) as a yellow solid. MS (ESI) calculated for (C₂₀H₁₇F₃N₄OS) [M+H]⁺, 419; found, 419. ¹H NMR (300 MHz, DMSO-d₆) δ 8.52 (s, 1H), 8.20-7.99 (m, 2H), 7.99-7.70 (m, 3H), 7.38 (t, J=7.8 Hz, 1H), 7.22-6.98 (m, 1H), 5.17 (s, 2H), 4.72 (q, J=6.9 Hz, 1H), 3.36 (s, 3H), 1.68 (d, J=6.9 Hz, 3H).

### (R)-2-(3-(1-(4-methyl-4H-1,2,4-triazol-3-ylthio)ethyl)phenyl)-4-(trifluoromethyl)isoindolin-1-one (166b)

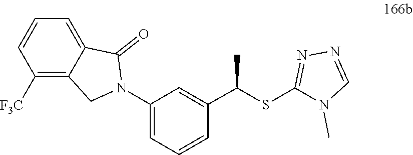 [SMILES: C[C@@H](Sc1nncn1C)c1cccc(N2Cc3c(cccc3C(F)(F)F)C2=O)c1]

To a solution of methyl 2-(bromomethyl)-3-(trifluoromethyl)benzoate (200 mg, 0.67 mmol), (R)-3-(1-(4-methyl-4H-1,2,4-triazol-3-ylthio)ethyl)aniline (157.7 mg, 0.67 mmol) in ethanol (1 mL) was added triethylamine (0.14 mL, 1.0 mmol). The solution was stirred overnight at 80° C., and then concentrated. The residue was puritify by prep-HPLC to afford 166b (80 mg, 28%) as a yellow solid. ¹H NMR (300 MHz, DMSO-d₆) δ 8.52 (s, 1H), 8.11-7.97 (m, 2H), 7.91-7.73 (m, 3H), 7.38 (t, J=7.9 Hz, 1H), 7.19-7.08 (m, 1H), 5.17 (s, 2H), 4.72 (q, J=6.9 Hz, 1H), 3.36 (s, 3H), 1.68 (d, J=6.9 Hz, 3H). MS (ESI) calculated for (C₂₀H₁₇F₃N₄OS) [M+H]⁺, 419; found, 419.

### Example 167: 4-chloro-2-[3-[(1S)-1-[(4-methyl-1,2,4-triazol-3-yl)sulfanyl]ethyl]phenyl]-3H-pyrrolo[3,4-c]pyridin-1-one (167)

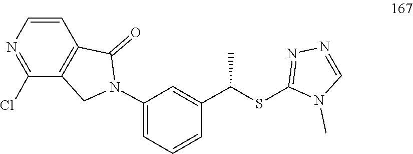 [SMILES: C[C@H](Sc1nncn1C)c1cccc(N2Cc3c(ccnc3Cl)C2=O)c1]

Step 1: Ethyl 3-(bromomethyl)-2-chloro-pyridine-4-carboxylate. To a stirred solution of ethyl 2-chloro-3-methyl-pyridine-4-carboxylate (2.37 g, 11.85 mmol) and N-bromosuccinimide (2.3 g, 13.0 mmol) in carbontetrachloride (40 mL) was added benzoyl peroxide (574 mg, 2.37 mmol). The mixture was stirred at 80° C. for about 14 h. The mixture was concentrated then purified by flash column chromatography eluting with 0-30% EtOAc in petroleum ether to afford the title compound (2.6 g, 9.33 mmol, 79% yield).

Step 2: 4-chloro-2-[3-[(1S)-1-[(4-methyl-1,2,4-triazol-3-yl)sulfanyl]ethyl]phenyl]-3H-pyrrolo[3,4-c]pyridin-1-one. To a stirred solution of ethyl 3-(bromomethyl)-2-chloro-pyridine-4-carboxylate (400 mg, 1.44 mmol) and 3-[(1S)-1-[(4-methyl-1,2,4-triazol-3-yl)sulfanyl]ethyl]aniline (336 mg, 1.44 mmol) in dimethylsulfoxide (9.7 mL) was added and triethylamine (0.21 mL, 1.51 mmol). The mixture was heated at about 60° C. for about 14 h. The mixture was cooled to about 25° C., concentrated, and the residue was purified by HPLC to afford compound 167 (0.26 g, 46% yield). ¹H NMR (500 MHz, DMSO-d₆) δ 8.66-8.64 (m, 1H), 8.54 (s, 1H), 7.90-7.86 (m, 2H), 7.83 (d, J=4.9 Hz, 1H), 7.41 (t, J=7.9 Hz, 1H), 7.18 (dt, J=7.8, 1.3 Hz, 1H), 5.10 (s, 2H), 4.74 (q, J=7.0 Hz, 1H), 3.39 (s, 3H), 1.70 (d, J=7.0 Hz, 3H). LCMS: C₁₈H₁₆ClN₅OS requires: 385, found: m/z=386 [M+H]⁺

### Example 168: 2-[3-fluoro-5-[(1R)-1-[(4-methyl-1,2,4-triazol-3-yl)sulfanyl]ethyl]phenyl]-4-(trifluoromethyl)isoindolin-1-one (168)

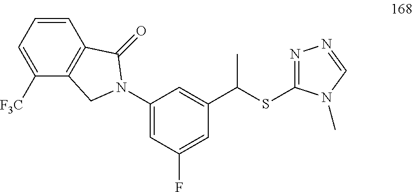 [SMILES: CC(Sc1nncn1C)c1cc(F)cc(N2Cc3c(cccc3C(F)(F)F)C2=O)c1]

Step 1: 2-(3-Acetyl-5-fluoro-phenyl)-4-(trifluoromethyl)isoindolin-1-one. To a stirring solution of Xantphos (0.06 g, 0.10 mmol), l-(3-bromo-5-fluorophenyl)ethanone (0.22 g, 0.99 mmol), 4-(trifluoromethyl)isoindolin-1-one (200 mg, 0.99 mmol), and cesium carbonate (0.66 g, 1.99 mmol) in dioxane (15 mL) was added palladium(II) acetate (10 mg, 0.05 mmol). The mixture was allowed to stir at 100-120° C. for about 4 h in a sealed vial and then cooled to rt. The reaction was diluted with water or 1 M hydrochloric acid. General Work-up Procedure 1 was followed. The crude was purified using flash column chromatography to yield the title compound (214 mg, 64% yield) as a colorless solid.

Step 2: 2-(4-fluoro-3-(l-hydroxyethyl)phenyl)-4-(trifluoromethyl)isoindolin-1-one. To a stirring solution of 2-(3-acetyl-4-fluoro-phenyl)-4-(trifluoromethyl)isoindolin-1-one (350 mg, 1.04 mmol) in methanol (2.5 mL) at rt was added sodium borohydride (80 mg, 2.08 mmol). The mixture was stirred for 1 h. The reaction was diluted with 1 M hydrochloric acid. General Work-up Procedure 1 was followed. The crude was used.

Step 3: 2-(3-fluoro-5-(1-((4-methyl-4H-1,2,4-triazol-3-yl)thio)ethyl)phenyl)-4-(trifluoromethyl)isoindolin-1-one. To a stirring solution of 2-(3-fluoro-5-(l-hydroxyethyl)phenyl)-4-(trifluoromethyl)isoindolin-1-one, 3-mercapto-4-methyl-4H-1,2,4-triazole (0.36 g, 3.11 mmol), and triphenylphosphine resin (1.38 g, 2.08 mmol) in THF (2.5 mL) at rt was added diisopropyl azodicarboxylate (0.41 mL, 2.08 mmol). The mixture was stirred for 24 h. The crude was purified using flash column chromatography to yield the title compound (63 mg, 14% yield). ¹H NMR (500 MHz, DMSO-d₆) δ 8.55 (s, 1H), 8.11 (d, J=7.6 Hz, 1H), 8.07 (dd, J=7.8, 0.9 Hz, 1H), 7.85 (dt, J=11.3, 2.2 Hz, 1H), 7.83-7.78 (m, 1H), 7.69 (t, J=1.8 Hz, 1H), 7.03 (dt, J=9.4, 1.7 Hz, 1H), 5.19 (s, 2H), 4.76 (q, J=7.0 Hz, 1H), 3.42 (s, 3H), 1.69 (d, J=7.0 Hz, 3H). LCMS: C₂₀H₁₆F₄N₄OS requires: 436, found: m/z=437 [M+H]⁺

### Step 4: 2-[3-fluoro-5-[(1S)-1-[(4-methyl-1,2,4-triazol-3-yl)sulfanyl]ethyl]phenyl]-4-(trifluoromethyl)isoindolin-1-one (168a) and 2-[3-fluoro-5-[(1R)-1-[(4-methyl-1,2,4-triazol-3-yl)sulfanyl]ethyl]phenyl]-4-(trifluoromethyl)isoindolin-1-one (168b)

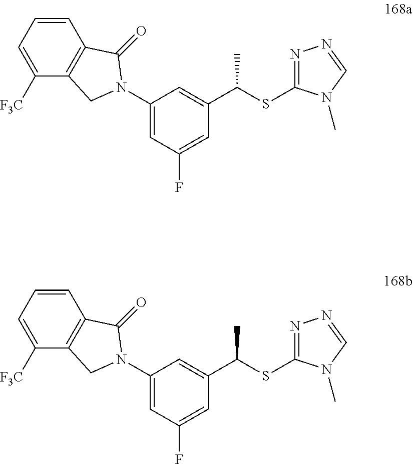 [SMILES: C[C@H](Sc1nncn1C)c1cc(F)cc(N2Cc3c(cccc3C(F)(F)F)C2=O)c1 ; C[C@@H](Sc1nncn1C)c1cc(F)cc(N2Cc3c(cccc3C(F)(F)F)C2=O)c1]

The mixture of enantiomers was purified by SFC to give 168a. ¹H NMR (500 MHz, DMSO-d₆) δ 8.55 (s, 1H), 8.11 (d, J=7.6 Hz, 1H), 8.07 (dd, J=7.8, 0.9 Hz, 1H), 7.85 (dt, J=11.3, 2.2 Hz, 1H), 7.83-7.78 (m, 1H), 7.69 (t, J=1.8 Hz, 1H), 7.03 (dt, J=9.4, 1.7 Hz, 1H), 5.19 (s, 2H), 4.76 (q, J=7.0 Hz, 1H), 3.42 (s, 3H), 1.69 (d, J=7.0 Hz, 3H). LCMS: C₂₀H₁₆F₄N₄OS requires: 436, found: m/z=437 [M+H]⁺; and 168b. ¹H NMR (500 MHz, DMSO-d6) δ 8.55 (s, 1H), 8.11 (d, J=7.6 Hz, 1H), 8.07 (dd, J=7.8, 0.9 Hz, 1H), 7.85 (dt, J=11.3, 2.2 Hz, 1H), 7.83-7.78 (m, 1H), 7.69 (t, J=1.8 Hz, 1H), 7.03 (dt, J=9.4, 1.7 Hz, 1H), 5.19 (s, 2H), 4.76 (q, J=7.0 Hz, 1H), 3.42 (s, 3H), 1.69 (d, J=7.0 Hz, 3H). LCMS: C₂₀H₁₆F₄N₄OS requires: 436, found: m/z=437 [M+H]⁺

### Example 169: 2-[2-fluoro-5-[(1S)-1-[(4-methyl-1,2,4-triazol-3-yl)sulfanyl]ethyl]phenyl]-4-(trifluoromethyl)isoindolin-1-one (169)

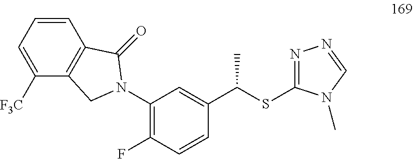 [SMILES: C[C@H](Sc1nncn1C)c1ccc(F)c(N2Cc3c(cccc3C(F)(F)F)C2=O)c1]

Compound 169 was obtained according to procedures disclosed herein. ¹H NMR (500 MHz, DMSO-d₆) δ 8.55 (s, 1H), 8.12 (d, J=7.6 Hz, 1H), 8.08 (dt, J=7.7, 0.9 Hz, 1H), 7.86-7.81 (m, 1H), 7.71-1 FI (m, 1H), 7.34-7.32 (m, 1H), 5.14 (s, 2H), 4.72 (q, J=7.0 Hz, 2H), 3.40 (s, 3H), 1.69 (d, J=7.0 Hz, 3H). LCMS: C₂₀H₁₆F₄N₄OS requires: 436, found: m/z=437 [M+H]⁺.

### Example 170: 2-[2-Fluoro-5-[(1R)-1-[(4-methyl-1,2,4-triazol-3-yl)sulfanyl]ethyl]phenyl]-4-(trifluoromethyl)isoindolin-1-one (170)

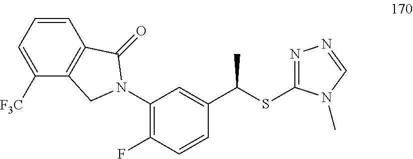 [SMILES: C[C@@H](Sc1nncn1C)c1ccc(F)c(N2Cc3c(cccc3C(F)(F)F)C2=O)c1]

The mixture of enantiomers was purified by SFC to give 2.1 mg of 170. ¹H NMR (500 MHz, DMSO-d6) δ 8.55 (s, 1H), 8.12 (d, J=7.6 Hz, 1H), 8.08 (dt, J=7.7, 0.9 Hz, 1H), 7.86-7.81 (m, 1H), 7.71-7.67 (m, 1H), 7.34-7.32 (m, 1H), 5.14 (s, 2H), 4.72 (q, J=7.0 Hz, 2H), 3.40 (s, 3H), 1.69 (d, J=7.0 Hz, 3H). LCMS: C₂₀H₁₆F₄N₄OS requires: 436, found: m/z=437 [M+H]⁺.

### Example 171: 2-(4-(1-((4-methyl-4H-1,2,4-triazol-3-yl)thio)ethyl)pyridin-2-yl)isoindolin-1-one (171)

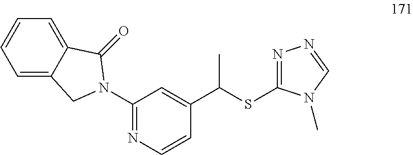 [SMILES: CC(Sc1nncn1C)c1ccnc(N2Cc3ccccc3C2=O)c1]

Compound 171 (450 mg, 32%) was obtained according to procedures disclosed herein as a light yellow solid. MS (ESI) calculated for (C₁₈H₁₇N₅OS) [M+H]⁺, 352; found, 352. ¹H NMR (400 MHz, DMSO-d₆) δ 8.55 (s, 2H), 8.37-8.36 (m, 1H), 7.83-7.81 (m, 1H), 7.74-7.69 (m, 2H), 7.58-7.54 (m, 1H), 7.15-7.13 (m, 1H), 5.08 (s, 2H), 4.79-4.73 (m, 1H), 3.48 (s, 3H), 1.68 (d, J=12 Hz, 3H).

### Example 172: (S)-(4-(1-((4-methyl-4H-1,2,4-triazol-3-yl)thio)ethyl)pyridin-2-yl)-4-(trifluoromethyl)isoindolin-1-one (172a) and (R)-2-(4-(1-((4-methyl-4H-1,2,4-triazol-3-yl)thio)ethyl)pyridin-2-yl)-4-(trifluoromethyl)isoindolin-1-one (172b)

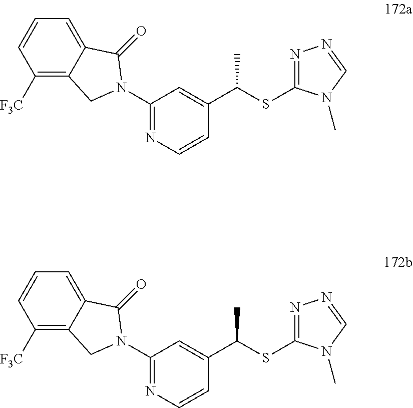 [SMILES: C[C@H](Sc1nncn1C)c1ccnc(N2Cc3c(cccc3C(F)(F)F)C2=O)c1 ; C[C@@H](Sc1nncn1C)c1ccnc(N2Cc3c(cccc3C(F)(F)F)C2=O)c1]

Step 1: (S)-1-(2-bromopyridin-4-yl)ethan-1-ol and (R)-1-(2-bromopyridin-4-yl)ethyl acetate: To a mixture of 1-(2-bromopyridin-4-yl)ethan-1-ol (2 g, 10 mmol) and vinyl acetate (8.6 g, 100 mmol) in diisopropyl ether (100 mL) was added Novozym 435 (200 mg, 10% w/w). The mixture was stirred at 35° C. for 36 h. The mixture was filtered and then concentrated. The residue was purified by flash chromatography on silica gel eluting with 20-50% EtOAc in petroleum ether to afford (S)-1-(2-bromopyridin-4-yl)ethan-1-ol (541 mg, e.e.=98.6%, 27%) as an off-white solid and (R)-1-(2-bromopyridin-4-yl)ethyl acetate (1.1 g) as a yellow oil.

Step 2: (R)-1-(2-bromopyridin-4-yl)ethan-1-ol. To a solution of (R)-1-(2-bromopyridin-4-yl) EtOAc (1.1 g, 4.5 mmol) in methanol (20 mL) was added potassium carbonate (1.2 g, 9.0 mmol) at 20° C. The mixture was stirred at rt for 2 h. The solid was filtered off, and the filtrate was evaporated in vacuo. The residue was purified by flash chromatography on silica gel eluting with 30-50% EtOAc in petroleum ether to afford the title compound (524 mg, e.e.=98.4%, 26%) as a colorless solid.

Step 3: (S)-2-(4-(1-hydroxyethyl)pyridin-2-yl)-4-(trifluoromethyl)isoindolin-1-one. To a mixture of 4-(trifluoromethyl)isoindolin-1-one (200 mg, 1.0 mmol), (S)-1-(2-bromopyridin-4-yl)ethan-1-ol (220 mg, 1.09 mmol), Xantphos (115 mg, 0.20 mmol) and cesium carbonate (972 mg, 2.98 mmol) in dioxane (5 mL) was added tris(dibenzylideneacetone)dipalladium(0) (91 mg, 0.1 mmol). The mixture was degassed with nitrogen and stirred at 80° C. for 2 h. When the reaction was complete, the reaction was quenched by the addition of water (20 mL). General Work-up Procedure 1 was followed to afford the residue, which was purified by flash chromatography with 10-50% EtOAc in petroleum ether to afford the title compound (170 mg, 53%) as a light yellow solid.

Step 4: (R)-2-(4-(1-((4-methyl-4H-1,2,4-triazol-3-yl)thio)ethyl)pyridin-2-yl)-4-(trifluoromethyl)isoindolin-1-one: To a stirred solution of (5)-2-(4-(l-hydroxyethyl)pyridin-2-yl)-4-(trifluoromethyl)isoindolin-1-one (150 mg, 0.47 mmol), 4-methyl-4H-1,2,4-triazole-3-thiol (64 mg, 0.56 mmol) and triphenylphosphine (183 mg, 0.70 mmol) in THF (3 mL) under nitrogen was added diisopropyl azodicarboxylate (141 mg, 0.70 mmol). The solution was stirred at rt overnight. The solution was concentrated to give the residue, which was purified by HPLC to afford the title compound (113 mg, 58%) as a colorless solid. MS (ESI) calculated for (C₁₉H₁₆F₃N₅OS) [M+H]⁺, 420; found, 420. ¹H NMR (400 MHz, DMSO-d₆) δ 8.54 (d, J=1.6 Hz, 2H), 8.40-8.39 (m, 1H), 8.14-8.10 (m, 2H), 7.86-7.77 (m, 1H), 7.19-7.18 (m, 1H), 5.25 (d, J=2.0 Hz, 2H), 4.77 (q, J=7.2 Hz, 1H), 3.46 (s, 3H), 1.68 (d, J=7.2 Hz, 3H).

Step 5: (R)-2-(4-(1-hydroxyethyl)pyridin-2-yl)-4-(trifluoromethyl)isoindolin-1-one: Followed Step 3 with (R)-1-(2-bromopyridin-4-yl)ethan-1-ol as a substrate to afford 172b (150 mg, 47%) as a light yellow solid. Step 6: (S)-2-(4-(1-((4-methyl-4H-1,2,4-triazol-3-yl)thio)ethyl)pyridin-2-yl)-4-(trifluoromethyl)isoindolin-1-one. Followed Step 4 to afford with (R)-2-(4-(1-hydroxyethyl)pyridin-2-yl)-4-(trifluoromethyl)isoindolin-1-one to afford 172a as colorless solid (90 mg, 50%). MS (ESI) calculated for (C₁₉H₁₆F₃N₅OS) [M+H]⁺, 420.1; found, 420.1 ¹H NMR (400 MHz, DMSO-d₆) δ 8.54 (d, J=3.6 Hz, 2H), 8.39 (d, J=5.2 Hz, 1H), 8.17-8.11 (m, 2H), 7.92-7.70 (m, 1H), 7.18 (d, J=5.2 Hz, 1H), 5.25 (s, 2H), 4.78-4.77 (m, 1H), 3.47 (d, J=3.6 Hz, 3H), 1.76-1.58 (m, 3H).

### Example 173: N-methyl-2-(4-[1-[(4-methyl-4H-1,2,4-triazol-3-yl)sulfanyl]ethyl]pyridin-2-yl)-3-oxo-2,3-dihydro-1H-isoindole-5-carboxamide (173)

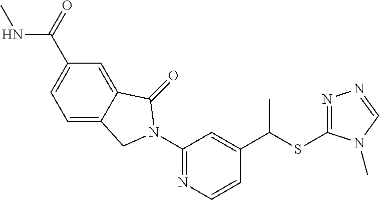 [SMILES: CNC(=O)c1ccc2c(c1)C(=O)N(c1cc(C(C)Sc3nncn3C)ccn1)C2]

Compound 173 (4.5 mg, 3%) was obtained according to procedures disclosed herein as a colorless solid using standard chromatography purification methods. MS (ESI) calculated for (C₂₀H₂₀N₆O₂S) [M+H]⁺, 409, found, 409. ¹H NMR (400 MHz, DMSO-d₆) δ 8.71 (s, 1H), 8.57 (s, 2H), 8.37 (d, J=5.6 Hz, 1H), 8.28 (s, 1H), 8.18 (d, J=6.8 Hz, 1H), 7.79 (d, J=8.4 Hz, 1H), 7.16 (d, J=5.2 Hz, 1H), 5.14 (s, 2H), 4.80-4.74 (m, 1H), 3.47 (s, 3H), 2.83 (d, J=4.0 Hz, 3H), 1.68 (d, J=7.2 Hz, 3H).

### Example 174: 2-[6-(2-hydroxyethylamino)-4-[1-[(4-methyl-1,2,4-triazol-3-yl)sulfanyl]ethyl]-2-pyridyl]-4-(trifluoromethyl)isoindolin-1-one (174)

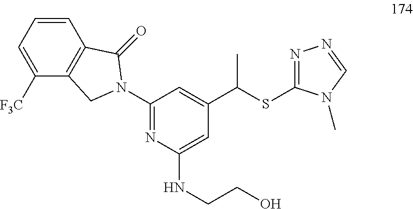 [SMILES: CC(Sc1nncn1C)c1cc(NCCO)nc(N2Cc3c(cccc3C(F)(F)F)C2=O)c1]

Compound 174 (12 mg, 0.03 mmol, 38% yield) was obtained according to procedures disclosed herein as a yellow oil: 1H NMR (500 MHz, DMSO-d₆) δ 8.57 (s, 1H), 8.06 (dd, J=22.0, 7.7 Hz, 2H), 7.79 (t, J=7.7 Hz, 1H), 7.70 (d, J=1.3 Hz, 1H), 6.24 (d, J=1.3 Hz, 2H), 5.18 (s, 2H), 4.56 (q, J=6.9 Hz, 1H), 3.56 (t, J=6.0 Hz, 2H), 3.48 (s, 3H), 3.32 (t, J=6.0 Hz, 2H), 1.60 (d, J=7.0 Hz, 3H). LCMS: C₂₁H₂₁F₃N₆O₂S requires: 478 found: m/z=479 [M+H]⁺

### Example 175: 2-[6-amino-4-[1-[(4-methyl-1,2,4-triazol-3-yl)sulfanyl]ethyl]-2-pyridyl]-4-(trifluoromethyl)isoindolin-1-one (175)

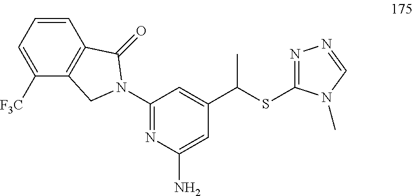 [SMILES: CC(Sc1nncn1C)c1cc(N)nc(N2Cc3c(cccc3C(F)(F)F)C2=O)c1]

Compound 175 (12 mg, 13% yield) was obtained according to procedures disclosed herein as a yellow oil: 1H NMR (500 MHz, DMSO-d6) δ 8.58 (s, 1H), 8.08 (d, J=7.6 Hz, 1H), 8.04 (d, J=7.7 Hz, 1H), 7.79 (t, J=7.7 Hz, 1H), 7.73 (d, J=1.4 Hz, 1H), 6.20 (d, J=1.3 Hz, 1H), 5.16 (s, 2H), 4.55 (q, J=6.9 Hz, 1H), 3.50 (s, 3H), 1.60 (d, J=6.9 Hz, 3H). LCMS: C₁₉H₁₇F₃N₆OS requires: 434 found: m/z=435 [M+H]⁺

### Example 176: 2-[6-[(2-hydroxy-1-methyl-ethyl)amino]-4-[1-[(4-methyl-1,2,4-triazol-3-yl)sulfanyl]ethyl]-2-pyridyl]-4-(trifluoromethyl)isoindolin-1-one (176)

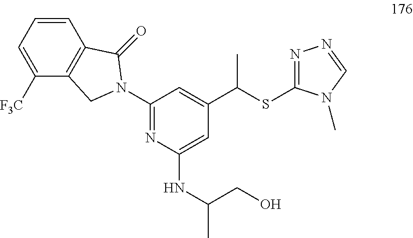 [SMILES: CC(CO)Nc1cc(C(C)Sc2nncn2C)cc(N2Cc3c(cccc3C(F)(F)F)C2=O)n1]

To a stirring solution of 2-[6-chloro-4-[1-[(4-methyl-1,2,4-triazol-3-yl)sulfanyl]-ethyl]-2-pyridyl]-4-(trifluoromethyl)iso-indolin-1-one (30 mg, 0.07 mmol) in dimethylsulfoxide (1 mL) was added DL-alaninol (0.1 mL, 1.32 mmol). The mixture was allowed to stir at 150° C. for about 6 h in a sealed vial and then cooled to rt. The residue was purified by HPLC to afford the title compound (1.6 mg, 5% yield) as a yellow oil: 1H NMR (500 MHz, methanol-d4) δ 8.57 (s, 1H), 8.20-8.05 (m, 1H), 8.03-7.90 (m, 1H), 7.76 (q, J=7.7, 6.8 Hz, 1H), 7.52 (s, 1H), 6.24 (d, J=1.2 Hz, 1H), 5.27-5.14 (m, 2H), 4.57 (q, J=7.1 Hz, 1H), 4.07-3.94 (m, 1H), 3.59 (d, J=6.5 Hz, 6H), 1.71 (d, J=7.0 Hz, 3H), 1.28-1.19 (m, 3H). LCMS: C₂₂H₂₃F₃N₆O₂S requires: 492 found: m/z=493 [M+H]⁺

### Example 177: 2-[6-[[(2S)-2-hydroxypropyl]amino]-4-[1-[(4-methyl-1,2,4-triazol-3-yl)sulfanyl]ethyl]-2-pyridyl]-4-(trifluoromethyl)iso-indolin-1-one (177)

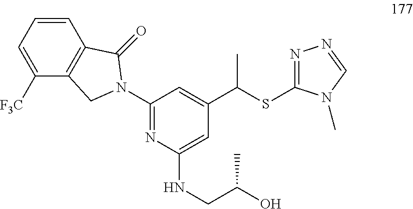 [SMILES: CC(Sc1nncn1C)c1cc(NC[C@H](C)O)nc(N2Cc3c(cccc3C(F)(F)F)C2=O)c1]

Compound 177 (23 mg, 71% yield) was obtained according to procedures disclosed herein as a yellow oil: 1H NMR (500 MHz, methanol-d4) δ 8.65 (s, 1H), 8.09 (d, J=7.7 Hz, 1H), 7.98 (d, J=7.7 Hz, 1H), 7.76 (t, J=7.8 Hz, 1H), 7.47 (d, J=1.3 Hz, 1H), 6.30 (t, J=1.3 Hz, 1H), 5.25-5.15 (m, 2H), 4.60 (p, J=7.4 Hz, 1H), 3.99 (pd, J=6.4, 4.3 Hz, 1H), 3.61 (s, 3H), 3.42 (ddd, J=13.6, 4.3, 1.7 Hz, 1H), 3.23 (ddd, J=13.6, 7.2, 4.4 Hz, 1H), 1.79-1.66 (m, 3H), 1.29-1.17 (m, 3H). LCMS: C₂₂H₂₃F₃N₆O₂S requires: 492 found: m/z=493 [M+H]⁺

### Example 178: 2-[6-(ethylamino)-4-[1-[(4-methyl-1,2,4-triazol-3-yl)sulfanyl]ethyl]-2-pyridyl]-4-(trifluoromethyl)isoindolin-1-one (178)

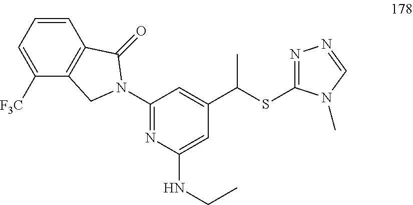 [SMILES: CCNc1cc(C(C)Sc2nncn2C)cc(N2Cc3c(cccc3C(F)(F)F)C2=O)n1]

The displacement reaction was carried out according to example 176 using 2-[6-chloro-4-[1-[(4-methyl-1,2,4-triazol-3-yl)sulfanyl]ethyl]-2-pyridyl]-4-(trifluoromethyl)isoindolin-1-one (30 mg, 0.07 mmol) and ethylamine (1 M in ethanol, 1 mL) to afforded the title compound 10 mg, 33% yield) as a yellow oil: 1H NMR (500 MHz, methanol-d4) δ 8.62 (s, 1H), 8.19-8.06 (m, 1H), 7.99 (d, J=7.7 Hz, 1H), 7.82-7.71 (m, 1H), 7.42 (s, 1H), 6.24 (s, 1H), 5.22 (s, 2H), 4.60 (q, J=7.0 Hz, 1H), 3.60 (s, 3H), 3.41-3.31 (m, 2H), 1.73 (d, J=7.0 Hz, 3H), 1.26 (t, J=7.2 Hz, 3H). LCMS: C₂₁H₂₁F₃N₆OS requires: 462 found: m/z=463 [M+H]⁺

### Example 179: 2-(4-(1-((4-methyl-4H-1,2,4-triazol-3-yl)thio)ethyl)-6-((tetrahydrofuran-3-yl)amino)pyridin-2-yl)-4-(trifluoromethyl)isoindolin-1-one (179)

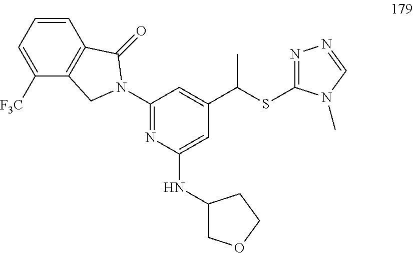 [SMILES: CC(Sc1nncn1C)c1cc(NC2CCOC2)nc(N2Cc3c(cccc3C(F)(F)F)C2=O)c1]

Compound 179 (5.6 mg, 17% yield) was obtained according to procedures disclosed herein as a yellow oil: 1H NMR (500 MHz, methanol-d4) δ 8.62 (d, J=3.4 Hz, 1H), 8.09 (d, J=7.7 Hz, 1H), 8.00-7.92 (m, 1H), 7.76 (dt, J=8.5, 6.0 Hz, 1H), 7.68 (d, J=11.3 Hz, 1H), 6.27-6.15 (m, 1H), 5.29-5.17 (m, 2H), 4.61 (dq, J=28.4, 7.1 Hz, 1H), 4.40 (ddt, J=7.6, 5.6, 3.8 Hz, 1H), 4.02 (dd, J=9.0, 5.8 Hz, 1H), 3.95 (q, J=7.6 Hz, 1H), 3.89-3.82 (m, 1H), 3.71 (ddd, J=8.9, 3.7, 1.8 Hz, 1H), 3.58 (m, 4H), 2.30 (dq, J=12.8, 7.6 Hz, 1H), 1.92 (dddd, J=12.9, 9.3, 4.8, 2.0 Hz, 1H), 1.73 (d, J=15.5, 7.0 Hz, 3H). LCMS: C₂₃H₂₃F₃N₆O₂S requires: 504 found: m/z=505 [M+H]⁺

### Example 180: 2-[6-(methylamino)-4-[1-[(4-methyl-1,2,4-triazol-3-yl)sulfanyl]ethyl]-2-pyridyl]-4-(trifluoromethyl)isoindolin-1-one (180)

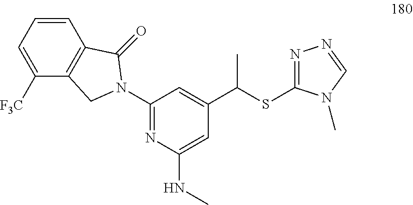 [SMILES: CNc1cc(C(C)Sc2nncn2C)cc(N2Cc3c(cccc3C(F)(F)F)C2=O)n1]

Compound 180 (28 mg, 90% yield) was obtained according to procedures disclosed herein as a yellow oil: 1H NMR (500 MHz, methanol-d4) δ 8.69 (s, 1H), 8.10 (d, J=7.6 Hz, 1H), 7.99 (dt, J=7.6, 0.9 Hz, 1H), 7.83-7.68 (m, 1H), 7.37 (s, 1H), 6.28 (d, J=1.3 Hz, 1H), 5.22 (s, 2H), 4.63 (q, J=7.1 Hz, 1H), 3.62 (s, 3H), 2.92 (s, 3H), 1.74 (d, J=7.0 Hz, 3H). LCMS: C₂₀H₁₉F₃N₆OS requires: 448 found: m/z=449 [M+H]⁺

### Example 181: N-(3-(3-hydroxy-1-((4-methyl-4H-1,2,4-triazol-3-yl)thio)propyl)phenyl)-6-(trifluoromethyl)picolinamide (181)

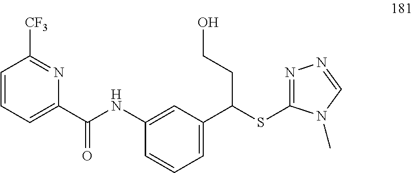 [SMILES: Cn1cnnc1SC(CCO)c1cccc(NC(=O)c2cccc(C(F)(F)F)n2)c1]

Compound 180 (9.1 mg, 18% yield) was obtained according to procedures disclosed herein. ¹H NMR (500 MHz, Methanol-d₄) δ 8.66 (d, J=2.1 Hz, 1H), 8.45 (d, J=7.9 Hz, 1H), 8.29 (t, J=7.9 Hz, 1H), 8.05 (dd, J=7.9, 1.0 Hz, 1H), 7.73 (t, J=2.0 Hz, 1H), 7.67 (ddd, J=8.1, 2.2, 1.0 Hz, 1H), 7.32 (t, J=7.9 Hz, 1H), 7.04 (dt, J=7.7, 1.3 Hz, 1H), 4.71 (t, J=7.6 Hz, 1H), 3.73 (dt, J=11.0, 6.0 Hz, 1H), 3.61-3.52 (m, 1H), 3.41 (s, 3H), 2.41-2.21 (m, 2H). LCMS: C₁₉H₁₈F₃N₅O₂S requires: 437 found: m/z=438 [M+H]⁺.

### Example 182: (R)-N-(3-(1-(4-methyl-4H-1,2,4-triazol-3-yl)propan-2-yl)phenyl)isoquinoline-3-carboxamide (182)

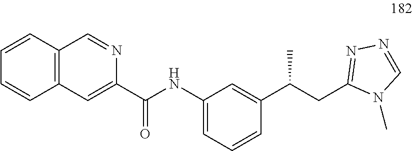 [SMILES: C[C@H](Cc1nncn1C)c1cccc(NC(=O)c2cc3ccccc3cn2)c1]

Step 1: Followed General procedure 6 (see Example 183) using D-1 to afford N-(3-(1-(4-methyl-4H-1,2,4-triazol-3-yl)propan-2-yl)phenyl)isoquinoline-3-carboxamide (37.2 mg, 22%) as a yellow semi-solid. ¹H NMR (300 MHz, DMSO-d₆) δ 10.67 (s, 1H), 9.48 (s, 1H), 8.71 (d, J=0.9 Hz, 1H), 8.37-8.22 (m, 3H), 7.99-7.81 (m, 4H), 7.30 (t, J=7.8 Hz, 1H), 7.02 (t, J=7.2 Hz, 1H), 3.47 (s, 3H), 3.31-3.18 (m, 1H), 3.00 (d, J=7.4 Hz, 2H), 1.31 (d, J=6.9 Hz, 3H). MS (ESI) calculated for (C₂₂H₂₁N₅O) [M+H]⁺, 372; found, 372.

Step 2: The racemic product was separated by chiral-SFC to afford 5.3 mg of 182. ¹H NMR (500 MHz, MeOH-d4) δ 9.35 (s, 1H), 8.65 (s, 1H), 8.26 (s, 1H), 8.21 (d, J=8.3 Hz, 1H), 8.12 (d, J=7.9 Hz, 1H), 7.88 (ddd, J=1.2, 7.0, 8.1 Hz, 1H), 7.82 (ddd, J=1.1, 7.0, 8.1 Hz, 1H), 7.72 (bt, J=1.8 Hz, 1H), 7.70 (ddd, J=0.9, 2.0, 8.0 Hz, 1H), 7.31 (t, J=7.8 Hz, 1H), 7.00 (bd, J=11 Hz, 1H), 3.43 (s, 3H), 3.33-3.36 (m, 1H), 3.15 (dd, 6.8, 14.9 Hz, 1H), 3.09 (dd, J=8.1, 14.9 Hz, 1H), 1.44 (d, J=6.9 Hz, 3H).

### Example 183: (R)-N-(3-(1-(4-methyl-4H-1,2,4-triazol-3-yl)propan-2-yl)phenyl)-6-(trifluoromethyl)picolinamide (183a)

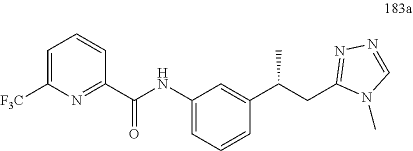 [SMILES: C[C@H](Cc1nncn1C)c1cccc(NC(=O)c2cccc(C(F)(F)F)n2)c1]

The amide bond formation reaction was carried out in a similar fashion as for 74 using D-a (1.87 g, 8.64 mmol) and 6-(trifluoromethyl)-4-(trifluoromethyl)pyridine-2-carboxylic acid (2.04 g, 10.4 mmol) to afforded the title compound (2.39 g, 71%). ¹H NMR (500 MHz, DMSO-d6) δ 10.33 (s, 1H), 8.45-8.32 (m, 2H), 8.30 (s, 1H), 8.19 (dd, J=7.5, 1.3 Hz, 1H), 7.79-7.70 (m, 2H), 7.31 (t, J=7.8 Hz, 1H), 7.05 (dt, J=7.7, 1.3 Hz, 1H), 3.46 (s, 3H), 3.28 (p, J=7.2 Hz, 1H), 3.00 (d, J=7.4 Hz, 2H), 1.30 (d, J=6.9 Hz, 3H). LCMS: C₁₉H₁₈F₃N₅O requires: 389, found: m/z=390 [M+H]⁺.

### (S)-N-(3-(1-(4-methyl-4H-1,2,4-triazol-3-yl)propan-2-yl)phenyl)-6-(trifluoromethyl)picolinamide (183b)

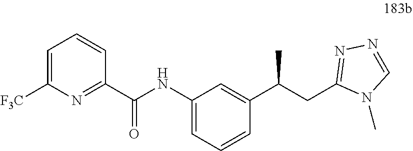 [SMILES: C[C@@H](Cc1nncn1C)c1cccc(NC(=O)c2cccc(C(F)(F)F)n2)c1]

Followed General procedure 6 using D-b (25 mg, 99%). ¹H NMR (500 MHz, Methanol-d₄) δ 8.83 (s, 1H), 8.45 (d, J=7.8 Hz, 1H), 8.29 (t, J=7.9 Hz, 1H), 8.06 (dd, J=7.8, 0.9 Hz, 1H), 7.69 (t, J=2.0 Hz, 1H), 7.62 (ddd, J=8.1, 2.2, 1.0 Hz, 1H), 7.34 (t, J=7.9 Hz, 1H), 7.06 (dt, J=7.7, 1.3 Hz, 1H), 3.61 (s, 3H), 3.43-3.21 (m, 3H), 1.48 (d, J=6.8 Hz, 3H). LCMS: C₁₉H₁₈F₃N₅O requires: 389, found: m/z=390 [M+H]⁺

### General Procedure 6:

To a mixture of D-a (100 mg, 0.46 mmol), acid (0.56 mmol), HATU (262 mg, 0.69 mmol) and N,N-diisopropylethylamine (118 mg, 0.92 mmol) in N,N-dimethylformamide (3 mL) was stirred at 25° C. for 2-24 h. The mixture was diluted by the addition of water, followed by General Work-up Procedure 1. The residue was purified by prep-HPLC or flash column chromatography to afford the desired product.

### Example 184: (R)-N-(3-(1-(4-methyl-4H-1,2,4-triazol-3-yl)propan-2-yl)phenyl)-4-(trifluoromethyl)picolinamide (184)

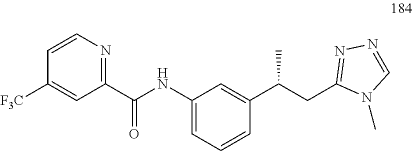 [SMILES: C[C@H](Cc1nncn1C)c1cccc(NC(=O)c2cc(C(F)(F)F)ccn2)c1]

Followed General procedure 6 to afford compound 184 (118 mg, 65%) as a colorless solid. ¹H NMR (300 MHz, DMSO-d₆) δ 10.74 (s, 1H), 9.05 (d, J=5.1 Hz, 1H), 8.37 (s, 1H), 8.30 (s, 1H), 8.13-8.11 (m, 1H), 7.84-7.81 (m, 2H), 7.33-7.28 (m, 1H), 7.05 (d, J=7.5 Hz, 1H), 3.46 (s, 3H), 3.30-3.23 (m, 1H), 2.98 (d, J=1.2 Hz, 2H), 1.30 (d, J=6.6 Hz, 3H). MS (ESI) calculated for (C₁₉H₁₈F₃N₅O) [M+H]⁺, 390; found, 390.

### Example 185: (R)-N-(3-(1-(4-methyl-4H-1,2,4-triazol-3-yl)propan-2-yl)phenyl)-6-(methylsulfonyl)picolinamide (185)

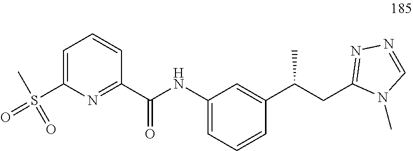 [SMILES: C[C@H](Cc1nncn1C)c1cccc(NC(=O)c2cccc(S(C)(=O)=O)n2)c1]

Followed General procedure 6 to afford compound 185 (101 mg, 55%) as a colorless solid. ¹H NMR (300 MHz, DMSO-d₆) δ 10.48 (s, 1H), 8.45-8.38 (m, 2H), 8.30-8.26 (m, 2H), 7.75-7.72 (m, 2H), 7.35-7.30 (m, 1H), 7.08 (d, J=7.8 Hz, 1H), 3.61 (s, 3H), 3.46 (s, 3H), 3.34-3.22 (m, 1H), 2.99 (d, J=7.5 Hz, 2H), 1.30 (d, J=6.9 Hz, 3H). MS (ESI) calculated for (C₁₉H₂₁N₅O₃S) [M+H]⁺, 400; found, 400.

### Example 186: (R)-5-chloro-4-(2-hydroxypropan-2-yl)-N-(3-(1-(4-methyl-4H-1,2,4-triazol-3-yl)propan-2-yl)phenyl)picolinamide (186)

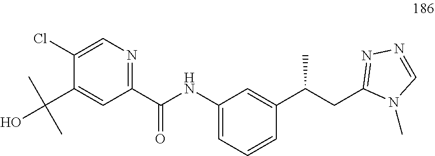 [SMILES: C[C@H](Cc1nncn1C)c1cccc(NC(=O)c2cc(C(C)(C)O)c(Cl)cn2)c1]

Followed General procedure 6 to afford compound 186 (104 mg, 54%) as a colorless solid. MS (ESI) calc'd for (C₂₁H₂₄ClN₅O₂) [M+H]⁺, 414; found, 414. ¹H NMR (300 MHz, DMSO-d₆) δ 10.55 (s, 1H), 8.65 (s, 1H), 8.58 (s, 1H), 8.32 (s, 1H), 7.80-7.78 (m, 2H), 7.27 (t, J=7.8 Hz, 1H), 7.02 (t, J=7.5 Hz, 1H), 5.79 (s, 1H), 3.46 (s, 3H), 3.24 (q, J=7.2 Hz, 1H), 2.98 (d, J=1.5 Hz, 2H), 1.62 (s, 6H), 1.29 (d, J=6.9 Hz, 3H).

### Example 187: (R)-N-(3-(1-(4-methyl-4H-1,2,4-triazol-3-yl)propan-2-yl)phenyl)-2-(trifluoromethyl)pyrimidine-4-carboxamide (187)

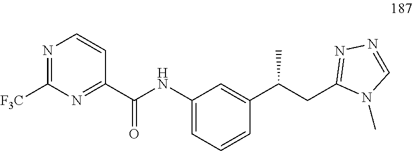 [SMILES: C[C@H](Cc1nncn1C)c1cccc(NC(=O)c2ccnc(C(F)(F)F)n2)c1]

Followed General procedure 6 to afford compound 187 (52 mg, 29%) as a yellow oil. MS (ESI) calculated for (C₁₈H₁₇F₃N₆O) [M+H]⁺, 391; found, 391. ¹H NMR (400 MHz, DMSO-d₆) δ 10.62 (s, 1H), 9.36 (d, J=5.2 Hz, 1H), 8.37 (d, J=5.2 Hz, 1H), 8.29 (s, 1H), 7.76-7.73 (m, 2H), 7.34-7.30 (m, 1H), 7.08 (d, J=7.6 Hz, 1H), 3.45 (s, 3H), 3.26-3.24 (m, 1H), 2.98 (d, J=7.6 Hz, 2H), 1.29 (d, J=6.8 Hz, 3H).

### Example 188: (R)-6-(1,1-difluoro-2-hydroxyethyl)-N-(3-(1-(4-methyl-4H-1,2,4-triazol-3-yl)propan-2-yl)phenyl)picolinamide (188)

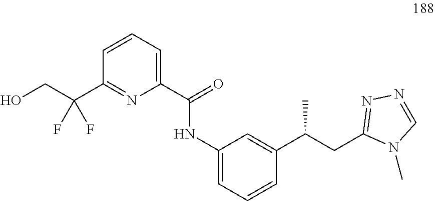 [SMILES: C[C@H](Cc1nncn1C)c1cccc(NC(=O)c2cccc(C(F)(F)CO)n2)c1]

Followed General procedure 6 to afford compound 188 (108 mg, 46%) as a light yellow solid. 1H NMR (400 MHz, DMSO-d₆) δ 10.29 (s, 1H), 8.34 (s, 1H), 8.31-8.20 (m, 2H), 7.98-7.95 (m, 1H), 7.79-7.69 (m, 2H), 7.33-7.29 (m, 1H), 7.06 (d, J=7.6 Hz, 1H), 5.56 (s, 1H), 4.31-4.24 (m, 2H), 3.47 (s, 3H), 3.32-3.23 (m, 1H), 3.01 (d, J=7.2 Hz, 2H), 1.30 (d, J=6.8 Hz, 3H). MS (ESI) calculated for (C₂₀H₂₁F₂N₅O₂) [M+H]⁺, 402, found, 402.

### Example 189: (R)-N-(3-(1-(4-methyl-4H-1,2,4-triazol-3-yl)propan-2-yl)phenyl)-6-(trifluoromethyl)pyrazine-2-carboxamide (189)

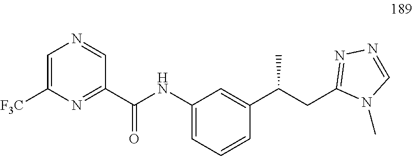 [SMILES: C[C@H](Cc1nncn1C)c1cccc(NC(=O)c2cncc(C(F)(F)F)n2)c1]

Followed General procedure 6 to afford compound 189 (93.7 mg, 57%) as an off-white solid. ¹H NMR (400 MHz, DMSO-d₆) δ 10.57 (s, 1H), 9.56 (s, 1H), 9.46 (s, 1H), 8.42 (s, 1H), 7.79-7.70 (m, 2H), 7.32 (t, J=7.8 Hz, 1H), 7.11-7.04 (m, 1H), 3.48 (s, 3H), 3.33-3.24 (m, 1H), 3.02 (d, J=7.4 Hz, 2H), 1.30 (d, J=6.9 Hz, 3H). MS (ESI) calc'd for (C₁₈H₁₇F₃N₆O) [M+H]⁺, 390; found, 390.

### Example 190: (R)-4-(3-hydroxyoxetan-3-yl)-N-(3-(1-(4-methyl-4H-1,2,4-triazol-3-yl)propan-2-yl)phenyl)-6-(trifluoromethyl)picolinamide (190)

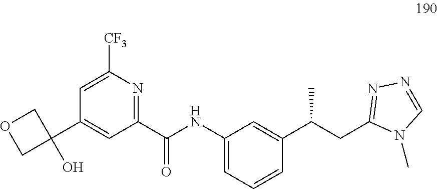 [SMILES: C[C@H](Cc1nncn1C)c1cccc(NC(=O)c2cc(C3(O)COC3)cc(C(F)(F)F)n2)c1]

Followed General procedure 6 to afford compound 190 (50 mg, 29%) as an off-white solid. 1H NMR (300 MHz, DMSO-d₆) δ 10.38 (s, 1H), 8.61 (s, 1H), 8.34 (s, 1H), 8.23 (d, J=1.5 Hz, 1H), 7.79-7.73 (m, 2H), 7.34-7.28 (m, 1H), 7.07-7.05 (m, 2H), 4.88 (d, J=7.2 Hz, 2H), 4.74 (d, J=6.9 Hz, 2H), 3.47 (s, 3H), 3.23-3.21 (m, 1H), 3.00 (d, J=7.5 Hz, 2H), 1.30 (d, J=6.9 Hz, 3H). MS (ESI) calculated for (C₂₂H₂₂F₃N₅O₃) [M+H]⁺, 462; found, 462.

### Example 191: (R)-4-cyclopropyl-N-(3-(1-(4-methyl-4H-1,2,4-triazol-3-yl)propan-2-yl)phenyl)-6-(trifluoromethyl)picolinamide (191)

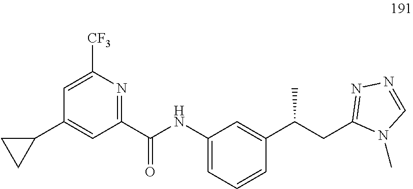 [SMILES: C[C@H](Cc1nncn1C)c1cccc(NC(=O)c2cc(C3CC3)cc(C(F)(F)F)n2)c1]

Step 1: 2-carboxy-6-(trifluoromethyl)pyridine 1-oxide. To a solution of 6-(trifluoromethyl)pyridine-2-carboxylic acid (1.0 g, 5.23 mmol) in trifluoroacetic acid (10 mL) was added hydrogen peroxide (0.9 g, 26.4 mmol, 30% in water) dropwise at rt. The mixture was stirred at 70° C. for 16 h and then concentrated to afford the title compound (0.7 g, crude) as a light yellow solid. Step 2: 2-(methoxycarbonyl)-6-(trifluoromethyl)pyridine 1-oxide. A mixture of 2-carboxy-6-(trifluoromethyl)pyridine 1-oxide (500 mg, 2.41 mmol) in hydrochloric acid solution (saturated in methanol, 5 mL) was stirred at rt for 16 h. The mixture was concentrated to afford the title compound (600 mg, crude) as a yellow oil. Step 3: methyl 4-chloro-6-(trifluoromethyl)-picolinate. A mixture of 2-(methoxycarbonyl)-6-(trifluoro-methyl)pyridine 1-oxide (600 mg, 2.71 mmol) and POCl₃ (3 mL) was stirred at 125° C. for 16 h. The mixture was concentrated. The residue was diluted with saturated sodium bicarbonate solution at 0° C. General Work-up Procedure 1 was followed. The residue was purified by flash chromatography on silica gel with 0-10% EtOAc in petroleum ether to afford the title compound (200 mg, 31%) as a light yellow oil. Step 4: 4-cyclopropyl-6-(trifluoromethyl)picolinic acid. To a solution of methyl 4-chloro-6-(trifluoromethyl)picolinate (2.0 g, 8.3 mmol) in toluene (20 mL) and water (0.2 mL) were added cyclopropylboronic acid (1.4 g, 16.7 mmol), Pd(AcO)₂ (94 mg, 0.42 mmol), tricyclohexyl phosphine (234 mg, 0.83 mmol) and potassium phosphate (7.1 g, 33.3 mmol). The mixture was stirred at reflux for 16 h under nitrogen, and then diluted with water. The aqueous layer was adjusted to pH ˜6 by 1 M hydrochloric acid. General Work-up Procedure 1 was followed. The residue was purified by flash chromatography on silica gel with 0-40% EtOAc in petroleum ether to afford the title compound (1.2 g, 61%) as a light yellow solid. Step 5: Synthesis of 191. Followed General procedure 6. After completion of the reaction, The mixture was purified by Prep-HPLC to afford compound 191 (202 mg, 54%) as a light yellow solid. 1H NMR (300 MHz, DMSO-d₆) δ 10.27 (s, 1H), 8.52 (s, 1H), 8.02-7.96 (m, 1H), 7.84-7.81 (m, 1H), 7.72-7.63 (m, 2H), 7.29 (t, J=7.8 Hz, 1H), 7.03 (d, J=7.8 Hz, 1H), 3.49 (s, 3H), 3.30-3.22 (m, 1H), 3.07-3.04 (m, 2H), 2.26-2.21 (m, 1H), 1.28 (d, J=6.9 Hz, 3H), 1.25-1.21 (m, 2H), 1.03-0.95 (m, 2H). MS (ESI) calc'd for (C₂₂H₂₂F₃N₅O) [M+H]⁺, 430; found 430.

### Example 192: (R)-4-methoxy-N-(3-(1-(4-methyl-4H-1,2,4-triazol-3-yl)propan-2-yl)phenyl)-6-(trifluoromethyl)picolinamide (192)

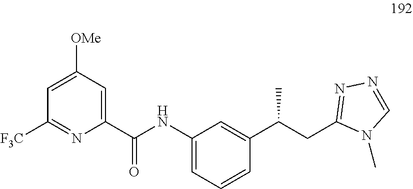 [SMILES: COc1cc(C(=O)Nc2cccc([C@H](C)Cc3nncn3C)c2)nc(C(F)(F)F)c1]

Step 1: Synthesis of 4-methoxy-6-(trifluoromethyl)picolinic acid. 4-chloro-6-(trifluoromethyl)picolinic acid (56 mg, 0.26 mmol) was dissolved in methanol (0.6 mL) sodium hydride (ca. 10 mg) was added and microwaved at 120° C. for 15 minutes and then at 110° C. for 15 minutes. 1 N Hydrochloric acid was added and the product was extracted with dichloromethane 3 times. The combined organic layers were dried. The solvent was removed under reduced pressure, to afford the title compound (57 mg) which was used without purification.

Step 2: Synthesis of 192. Followed General procedure 6 using D-a and the foregoing crude product to afford compound 192. ¹H NMR (500 MHz, DMSO-d₆) δ 10.27 (s, 1H), 8.86 (s, 1H), 7.85 (d, J=2.4 Hz, 1H), 7.78-7.74 (m, 1H), 7.72 (dd, J=6.6, 2.1 Hz, 2H), 7.31 (t, J=7.9 Hz, 1H), 7.06 (dd, J=7.8, 1.5 Hz, 1H), 4.04 (s, 3H), 3.59 (s, 3H), 3.30 (q, J=7.1 Hz, 1H), 3.15 (dd, J=7.4, 2.4 Hz, 2H), 1.32 (d, J=6.8 Hz, 3H). LCMS: C₂₀H₂₀F₃N₅O₂ requires: 419, found: m/z 420 [M+H]⁺.

### Example 193: (R)-2,6-dicyclopropyl-N-(3-(1-(4-methyl-4H-1,2,4-triazol-3-yl)propan-2-yl)phenyl)pyrimidine-4-carboxamide (193)

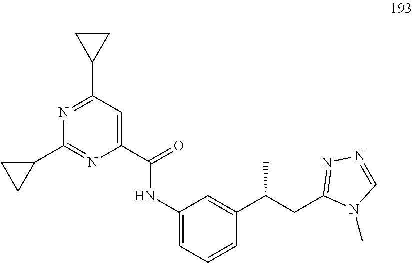 [SMILES: C[C@H](Cc1nncn1C)c1cccc(NC(=O)c2cc(C3CC3)nc(C3CC3)n2)c1]

Step 1: Synthesis of Methyl 2,6-dicyclopropylpyrimidine-4-carboxylate. To a solution of methyl 2,6-dichloropyrimidine-4-carboxylate (500 mg, 2.42 mmol) and tetrakis(triphenylphosphine)palladium(0) (0.28 g, 0.24 mmol) in THF (10 mL) was added bromo(cyclopropyl)zinc (14.5 mL of 0.5 M solution in THF, 7.3 mmol). The solution was heated at 60-65° C. for 19 h. The solution was allowed to cool to rt, then poured into saturated aqueous ammonium chloride (50 mL) and extracted with EtOAc. The combined organic phases were dried, filtered and concentrated onto Celite. Purification was by silica gel chromatography using 0-50% EtOAc/hex to afford the title compound (218 mg) as a yellow solid.

Step 2: Synthesis of 2,6-dicyclopropylpyrimidine-4-carboxylic acid. A mixture of methyl 2,6-dicyclopropylpyrimidine-4-carboxylate (218 mg, 1.0 mmol), lithium hydroxide monohydrate (130 mg, 3.0 mmol) in THF (3 mL) and water (0.3 mL) was stirred for 4 h. The mixture was poured into 0.2 N aqueous hydrochloric acid. General Work-up Procedure 1 was followed to afford the title compound (130 mg) as a yellow solid.

Step 3: Synthesis of 193e. Followed General procedure 6. The mixture was purified by HPLC to afford the title compound as a yellow solid: ¹H NMR (500 MHz, DMSO-d₆) δ 10.37 (s, 1H), 8.99 (s, 1H), 7.81-7.67 (m, 3H), 7.37-7.26 (m, 1H), 7.07 (dd, J=7.8, 1.4 Hz, 1H), 3.63 (s, 3H), 3.36-3.25 (m, 1H), 3.19 (dd, J=7.5, 3.3 Hz, 2H), 2.31-2.19 (m, 2H), 1.33 (d, J=6.8 Hz, 3H), 1.18-0.98 (m, 8H); LCMS: C₂₃H₂₆N₆O requires: 402, found:403 [M+H]⁺.

### Example 194: (R)-6-methyl-N-(3-(1-(4-methyl-4H-1,2,4-triazol-3-yl)propan-2-yl)phenyl)-2-(trifluoromethyl)pyrimidine-4-carboxamide (194)

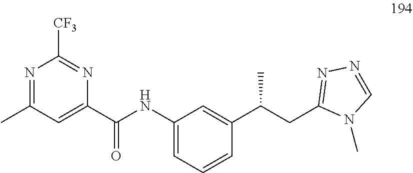 [SMILES: Cc1cc(C(=O)Nc2cccc([C@H](C)Cc3nncn3C)c2)nc(C(F)(F)F)n1]

Followed Example 193, step 3 to afford the title compound as a colorless solid: ¹H NMR (500 MHz, DMSO-d₆) δ 10.54 (s, 1H), 8.98 (s, 1H), 8.27 (s, 1H), 7.75 (ddd, J=8.1, 2.1, 1.0 Hz, 1H), 7.71 (t, J=1.9 Hz, 1H), 7.33 (t, J=7.9 Hz, 1H), 7.10 (dt, J=7.6, 1.3 Hz, 1H), 3.63 (s, 3H), 3.30 (p, J=7.0 Hz, 1H), 3.25-3.10 (m, 2H), 2.73 (s, 3H), 1.33 (d, J=6.9 Hz, 3H); LCMS: C₁₉H₁₉N₆O requires: 404, found: m/z=405 [M+H]⁺.

### Example 195: (R)-2-cyclopropyl-6-methyl-N-(3-(1-(4-methyl-4H-1,2,4-triazol-3-yl)propan-2-yl)phenyl)pyrimidine-4-carboxamide (195)

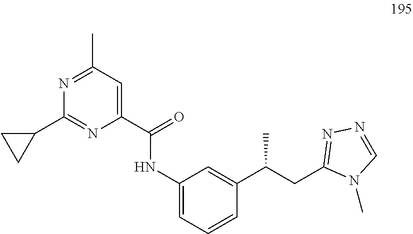 [SMILES: Cc1cc(C(=O)Nc2cccc([C@H](C)Cc3nncn3C)c2)nc(C2CC2)n1]

Followed Example 193, step 3 using 2-cyclopropyl-6-methyl-pyrimidine-4-carboxylic acid to afford the title compound as a colorless solid: 1H NMR (500 MHz, DMSO-d₆) δ 10.40 (s, 1H), 9.05 (s, 1H), 7.74 (dd, J=8.8, 7.3 Hz, 3H), 7.35-7.26 (m, 1H), 7.08 (dt, J=7.6, 1.4 Hz, 1H), 3.65 (s, 3H), 3.32 (h, J=6.9 Hz, 1H), 3.26-3.16 (m, 2H), 2.53 (s, 3H), 2.34 (tt, J=8.1, 4.8 Hz, 1H), 1.34 (d, J=6.8 Hz, 3H), 1.18 (dt, J=4.8, 2.9 Hz, 2H), 1.15-1.09 (m, 2H); LCMS: C₂₁H₂₄N₆O requires: 376, found: m/z=377 [M+H]⁺.

### Example 196: N-[3-[(1R)-1-methyl-2-(4-methyl-1,2,4-triazol-3-yl)ethyl]phenyl]-4-(trifluoromethyl)-1H-benzimidazole-2-carboxamide (196)

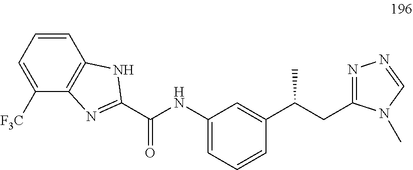 [SMILES: C[C@H](Cc1nncn1C)c1cccc(NC(=O)c2nc3c(C(F)(F)F)cccc3[nH]2)c1]

Step 1: Synthesis of 4-(trifluoromethyl)-1H-benzo[d]imidazole-2-carboxylic acid. A mixture of methyl 4-(trifluoromethyl)-1H-benzimidazole-2-carboxylate (600 mg, 2.5 mmol), lithium hydroxide monohydrate (320 mg, 7.4 mmol) THF (5 mL) and water (2 mL) was stirred for 6 h. The mixture was poured into 1.0 N aqueous hydrochloric acid and the resulting precipitate was collected to afford 512 mg of the title compound as a colorless solid.

Step 2: Synthesis of 196. Followed Example 193, step 3 to afford the title compound as a colorless solid: ¹H NMR (500 MHz, DMSO-d₆) δ 10.50 (s, 1H), 8.99 (s, 1H), 7.93 (d, J=7.3 Hz, 1H), 7.83 (ddd, J=8.1, 2.1, 1.0 Hz, 1H), 7.77 (t, J=1.9 Hz, 1H), 7.70 (d, J=7.5 Hz, 1H), 7.53 (t, J=7.9 Hz, 1H), 7.33 (t, J=7.9 Hz, 1H), 7.08 (dt, J=7.7, 1.3 Hz, 1H), 3.64 (s, 3H), 3.37-3.27 (m, 1H), 3.25-3.16 (m, 2H), 1.35 (d, J=6.8 Hz, 3H); LCMS: C₂₁H₁₉F₃N₆O requires: 428, found: m/z=429 [M+H]⁺.

### Example 197: (R)-1-methyl-N-(3-(1-(4-methyl-4H-1,2,4-triazol-3-yl)propan-2-yl)phenyl)-1H-pyrazole-3-carboxamide (197)

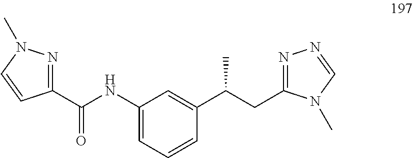 [SMILES: C[C@H](Cc1nncn1C)c1cccc(NC(=O)c2ccn(C)n2)c1]

Followed General procedure 6 to afford the title compound (43 mg, 95%) as an off-white solid. ¹H NMR (500 MHz, DMSO-d₆) δ 9.91 (s, 1H), 8.97 (s, 1H), 7.86 (d, J=2.3 Hz, 1H), 7.75-7.63 (m, 2H), 7.25 (t, J=7.8 Hz, 1H), 6.98 (dt, J=7.7, 1.3 Hz, 1H), 6.75 (d, J=2.3 Hz, 1H), 3.97 (s, 3H), 3.61 (d, J=4.3 Hz, 3H), 3.27 (dt, J=14.1, 7.0 Hz, 1H), 3.23-3.10 (m, 2H), 1.32 (d, J=6.8 Hz, 3H). LCMS: C₁₇H₂₀N₆O requires: 324, found: m/z=325 [M+H]⁺.

### Example 198: 6-cyclopropyl-N-[3-[(1R)-1-methyl-2-(4-methyl-1,2,4-triazol-3-yl)ethyl]phenyl]-4-methylsulfonyl-pyridine-2-carboxamide (198)

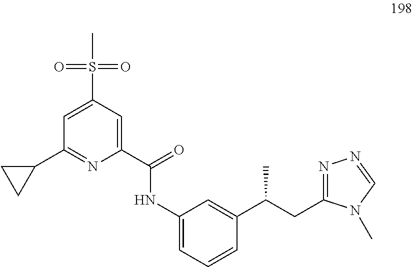 [SMILES: C[C@H](Cc1nncn1C)c1cccc(NC(=O)c2cc(S(C)(=O)=O)cc(C3CC3)n2)c1]

Step 1: Methyl 4-chloro-6-cyclopropylpicolinate. To a solution of methyl 4,6-dichloropyridine-2-carboxylate (1.0 g, 4.9 mmol) and tetrakis(triphenylphosphine)palladium(0) (0.34 g, 0.29 mmol) in THF (11 mL) was added bromo(cyclopropyl)zinc (11 mL of 0.5 M solution in THF, 5.6 mmol). The solution was heated at 60-65° C. for 24 h. The solution was allowed to cool to rt, then poured into saturated aqueous ammonium chloride and extracted with EtOAc. The combined organic phases were dried, filtered and concentrated onto Celite. Purification was by silica gel chromatography using 0-15% EtOAc/hex to afford the title compound (600 mg) as a colorless solid.

Step 2: Methyl 6-cyclopropyl-4-(methylsulfonyl)picolinate. A mixture of methyl 4-chloro-6-cyclopropyl-pyridine-2-carboxylate (270 mg, 1.3 mmol), sodium methane sulfonate (232 mg, 1.9 mmol), tetrabutyl ammonium chloride (100 mg, 0.38 mmol) in dimethylacetamide (1 mL) was heated at 100° C. for 19 h. The solution was allowed to cool to rt, then poured into saturated aqueous ammonium chloride (25 mL) and extracted with EtOAc. The combined organic phases were dried, filtered and concentrated onto Celite. Purification by silica gel chromatography using 0-100% EtOAc/hex to afford the title compound (159 mg) as a colorless solid.

Step 3: Synthesis of 6-cyclopropyl-4-(methylsulfonyl)picolinic acid. A mixture of methyl 6-cyclopropyl-4-methylsulfonyl-pyridine-2-carboxylate (155 mg, 0.61 mmol), lithium hydroxide monohydrate (78 mg, 1.8 mmol) THF (3 mL) and water (0.3 mL) was stirred at rt for 4 h. The mixture was poured into 0.2 N aqueous hydrochloric acid (20 mL), followed by General Work-up Procedure 1 to afford the title compound (150 mg) as a tan solid.

Step 4: Synthesis of 198. Followed Example 193, step 3 using D-a and the product of Step 3 to afford the title compound as a colorless solid. 1H NMR (500 MHz, DMSO-d₆) δ 10.26 (s, 1H), 9.02 (s, 1H), 8.23 (d, J=1.6 Hz, 1H), 8.06 (d, J=1.6 Hz, 1H), 7.77-7.68 (m, 2H), 7.38-7.30 (m, 1H), 7.10 (dt, J=7.7, 1.4 Hz, 1H), 3.65 (s, 3H), 3.41 (s, 3H), 3.36-3.31 (m, 1H), 3.21 (dd, J=7.4, 3.1 Hz, 2H), 2.46 (dt, J=8.2, 4.8 Hz, 1H), 1.35 (d, J=6.9 Hz, 3H), 1.31 (dt, J=6.3, 3.2 Hz, 2H), 1.19-1.13 (m, 2H); LCMS: C₂₂H₂₅N₅O₃S requires: 439, found: m/z=440 [M+H]⁺.

### Example 199: (R)-2-cyclopropyl-N-(3-(1-(4-methyl-4H-1,2,4-triazol-3-yl)propan-2-yl)phenyl)pyrimidine-4-carboxamide (199)

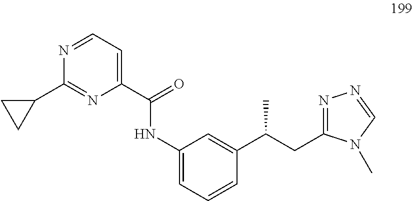 [SMILES: C[C@H](Cc1nncn1C)c1cccc(NC(=O)c2ccnc(C3CC3)n2)c1]

Followed Example 193, step 3 to afford the title compound as a yellow solid: ¹H NMR (500 MHz, DMSO-d₆) δ 10.46 (s, 1H), 9.10 (s, 1H), 8.93 (d, J=4.9 Hz, 1H), 7.82 (d, J=5.0 Hz, 1H), 7.78-7.67 (m, 2H), 7.33 (t, J=7.9 Hz, 1H), 7.08 (dt, J=7.6, 1.3 Hz, 1H), 3.66 (s, 3H), 3.35-3.28 (m, 1H), 3.24-3.19 (m, 2H), 2.38 (ddd, J=8.1, 5.6, 3.2 Hz, 1H), 1.34 (d, J=6.8 Hz, 3H), 1.23-1.17 (m, 2H), 1.17-1.12 (m, 2H); LCMS: C₂₀H₂₂N₆O requires: 362, found: m/z=363 [M+H]⁺.

### Example 200: (R)-N-(3-(1-(4-methyl-4H-1,2,4-triazol-3-yl)propan-2-yl)phenyl)-4-sulfamoyl-6-(trifluoromethyl)picolinamide (200)

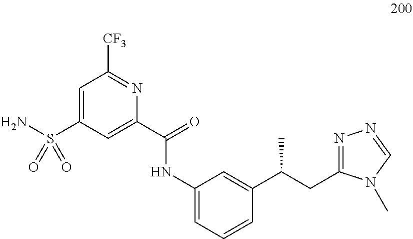 [SMILES: C[C@H](Cc1nncn1C)c1cccc(NC(=O)c2cc(S(N)(=O)=O)cc(C(F)(F)F)n2)c1]

Step 1: methyl 4-(benzylthio)-6-(trifluoromethyl)picolinate. Methyl 4-chloro-6-(trifluoromethyl)picolinate (500 mg, 2.1 mmol) was dissolved in N,N-dimethylformamide (2 mL). 1,8-Diazabicyclo[5.4.0]undec-7-ene (344 μL, 2.3 mmol) was added followed by benzyl mercaptan (244 μL, 2.1 mmol). Methyltert-butyl ether and water was added and the product was extracted with methyltert-butyl ether three times. The combined organic layers were dried, and concentrated to give title compound (686 mg) which was used without purification.

Step 2: methyl 4-(chlorosulfonyl)-6-(trifluoromethyl)picolinate. Methyl 4-(benzylthio)-6-(trifluoromethyl)picolinate (686 mg) was dissolved in dichloromethane (7 mL) at 0° C. Acetic acid (1 mL) and water (2.1 mL) was added. Dichlorodimethylhydantoin (826 mg, 4.2 mmol) was added in portions and the reaction was warmed up to rt. An additional portion of dichlorodimethylhydantoin (263 mg, 1.3 mmol) was added and the reaction was stirred overnight. Water was added and the product was extracted with dichloromethane. The combined organic layers were dried and concentrated under reduced pressure. The crude product was purified by silica gel column chromatography using a gradient of EtOAc in dichloromethane (0 to 50%) to give the title compound (473 mg, 75% yield over 2 steps).

Step 3: methyl 4-sulfamoyl-6-(trifluoromethyl)picolinate. Methyl 4-(chloro-sulfonyl)-6-(trifluoromethyl)picolinate (62 mg, 0.21 mmol) was dissolved in THF (0.3 mL). Ammonia solution (200 μL, 7 M in methanol, 1.4 mmol) was added followed by pyridine (40 μL, 0.49 mmol). The reaction was stirred for 2 h. 1 N hydrochloric acid and dichloromethane was added and the product was extracted with dichloromethane. The combined organic layers were dried and solvent removed under reduced pressure. The crude material was purified by silica gel column chromatography using a gradient of EtOAc in dichloromethane (0 to 50%) to give the title compound (33 mg, 57% yield).

Step 4: 4-sulfamoyl-6-(trifluoromethyl)picolinic acid lithium salt. Methyl 4-sulfamoyl-6-(trifluoromethyl)picolinate (33 mg, 0.12 mmol) was dissolved in THF (0.45 mL). 1 N lithium hydroxide solution (140 μL, 0.14 mmol) was added followed by methanol (0.14 mmol). The reaction was stirred for several hours and then the reaction mixture was lyophilized to give crude title compound.

Step 5: (R)-N-(3-(1-(4-methyl-4H-1,2,4-triazol-3-yl)propan-2-yl)phenyl)-4-sulfamoyl-6-(trifluoromethyl) picolinamide. This compound was synthesized in the same manner as 118 using crude 4-sulfamoyl-6-(trifluoromethyl)picolinic acid lithium salt (<0.12 mmol), D-a (24 mg, 0.11 mmol), HATU (49 mg, 0.13 mmol) and N,N-diisopropylethylamine (61 μL, 0.35 mmol) in N,N-dimethylformamide (0.45 mL). Compound 200 (1.7 mg, 3%) was obtained as a trifluoroacetate salt. ¹H NMR (500 MHz, Methanol-d₄) δ 10.29 (s, 1H), 8.82 (d, J=1.5 Hz, 1H), 8.67 (s, 1H), 8.40 (d, J=1.5 Hz, 1H), 7.75-7.68 (m, 1H), 7.68-7.62 (m, 1H), 7.35 (t, J=7.9 Hz, 1H), 7.07 (d, J=7.7 Hz, 1H), 3.57 (s, 3H), 1.47 (d, J=6.9 Hz, 3H). LCMS: C₁₉H₁₉F₃N₆O₃S requires: 468, found: m/z 469 [M+H]⁺. (−5H)

### Example 201: (R)-N-(3-(1-(4-methyl-4H-1,2,4-triazol-3-yl)propan-2-yl)phenyl)-4-(N-methylsulfamoyl)-6-(trifluoromethyl)picolinamide (201)

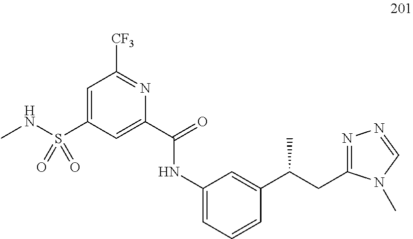 [SMILES: CNS(=O)(=O)c1cc(C(=O)Nc2cccc([C@H](C)Cc3nncn3C)c2)nc(C(F)(F)F)c1]

Step 1: Synthesis of methyl 4-(N-methylsulfamoyl)-6-(trifluoromethyl)picolinate. Methyl 4-(chlorosulfonyl)-6-(trifluoromethyl)picolinate (54 mg, 0.18 mmol) was dissolved in THF (0.5 mL). Methylamine solution (100 μL, 2 M in THF, 0.20 mmol) was added followed by addition of pyridine (40 μL, 0.49 mmol). The reaction was stirred overnight. 1 N hydrochloric acid and dichloromethane was added and the product was extracted with dichloromethane three times. The combined organic layers were dried, followed by removal of solvent under reduced pressure. The crude material was purified by silica gel column chromatography using a gradient of EtOAc in dichloromethane (0 to 30%) to give the title compound (30 mg, 57% yield).

Step 2: Synthesis of 4-(N-methylsulfamoyl)-6-(trifluoromethyl)picolinic acid lithium salt. This compound was synthesized in the same manner as step 4 of 200.

Step 3: (R)-N-(3-(1-(4-methyl-4H-1,2,4-triazol-3-yl)propan-2-yl)phenyl)-4-(N-methylsulfamoyl)-6-(trifluoromethyl)picolinamide. This compound was synthesized in the same manner as step 1 of 118 using crude 4-(N-methylsulfamoyl)-6-(trifluoromethyl)picolinic acid lithium salt (<0.10 mmol), D-a (22 mg, 0.10 mmol), HATU (43 mg, 0.11 mmol) and N,N-diisopropylethylamine (53 μL, 0.30 mmol) in N,N-dimethylformamide (0.45 mL). Compound 201 was obtained as a trifluoroacetate salt (27 mg, 45%). ¹H N M R (500 MHz, Methanol-d₄) δ 10.31 (s, OH), 8.81 (s, 1H), 8.74 (d, J=1.4 Hz, 1H), 8.34 (d, J=1.5 Hz, 1H), 7.71 (q, J=1.8 Hz, 1H), 7.67-7.61 (m, 1H), 7.36 (t, J=7.9 Hz, 1H), 7.09 (d, J=8.0 Hz, 1H), 3.61 (s, 3H), 2.66 (s, 3H), 1.49 (d, J=6.8 Hz, 3H). LCMS: C₂₀H₂₁F₃N₆O₃S requires: 482, found: m/z 483 [M+H]⁺. (−5H)

### Example 202: 6-(2-hydroxyethyl)-N-[3-[(2R)-1-(4-methyl-4H-1,2,4-triazol-3-yl)propan-2-yl]phenyl]pyridine-2-carboxamide (202)

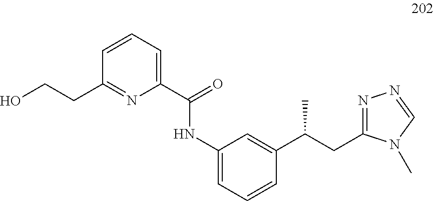 [SMILES: C[C@H](Cc1nncn1C)c1cccc(NC(=O)c2cccc(CCO)n2)c1]

Step 1: 6-(2-hydroxyethyl)pyridine-2-carboxylic acid. A solution of 6-(2-hydroxyethyl)pyridine-2-carbonitrile (Brito, J. A. et. al., Cur. Inorg. Chem., 2011, 1, 131) (6.0 g, 40.50 mmol) in conc, hydrochloric acid (60 mL, 12 M) was stirred at 110° C. for 16 h., The mixture was cooled to rt and evaporated in vacuo to afford the title compound (4.5 g, crude) as a colorless solid, which was used without purification. MS (ESI) calculated for (C₈H₉NO₃) [M+H]⁺, 168.1; found, 167.8.

Step 2: 6-(2-hydroxyethyl)-N-[3-[(2R)-1-(4-methyl-4H-1,2,4-triazol-3-yl)propan-2-yl]phenyl]pyridine-2-carboxamide. Followed General procedure 6. The crude product was purified by HPLC to afford compound 202 (98 mg, 9%) as a colorless solid. 1H NMR (300 MHz, DMSO-d₆) δ 10.35 (s, 1H), 8.30 (s, 1H), 8.00-7.93 (m, 2H), 7.77-7.74 (m, 2H), 7.58 (d, J=6.3 Hz, 1H), 7.29 (t, J=7.8 Hz, 1H), 7.04 (d, J=7.8 Hz, 1H), 4.71 (s, 1H), 3.90-3.85 (m, 2H), 3.45 (s, 3H), 3.38-3.22 (m, 1H), 3.07-2.98 (m, 4H), 1.29 (d, J=6.9 Hz, 3H). MS (ESI) calculated for (C₂₀H₂₃N₅O₂) [M+H]⁺, 366; found, 366.

### Example 203: 6-cyclopropyl-5-(hydroxymethyl)-N-[3-[(2R)-1-(4-methyl-4H-1,2,4-triazol-3-yl)propan-2-yl]phenyl]pyridine-2-carboxamide (203)

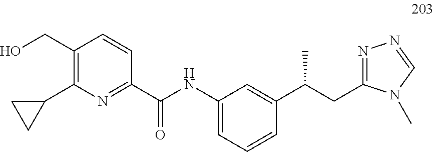 [SMILES: C[C@H](Cc1nncn1C)c1cccc(NC(=O)c2ccc(CO)c(C3CC3)n2)c1]

Step 1: Synthesis of 203. Followed General procedure 6. The mixture was purified by reverse phase flash column chromatography to afford compound 203 (32 mg, 10%) as an off-white solid. ¹H NMR (400 MHz, DMSO-d₆) δ 10.00 (s, 1H), 8.28 (s, 1H), 7.95-7.88 (m, 2H), 7.68-7.66 (m, 2H), 7.31-7.27 (m, 1H), 7.02 (d, J=7.6 Hz, 1H), 5.50-5.47 (m, 1H), 4.78 (d, J=5.2 Hz, 2H), 3.46 (s, 3H), 3.26-3.20 (m, 1H), 2.98 (d, J=7.2 Hz, 2H), 2.24-2.19 (m, 1H), 1.30-1.24 (m, 5H), 1.04-0.99 (m, 2H). MS (ESI) calculated for (C₂₂H₂₅N₅O₂) [M+H]⁺, 392, found, 392.

### Example 204: 4-((1R,3S)-3-hydroxycyclopentylamino)-N-(3-((R)-1-(4-methyl-4H-1,2,4-triazol-3-yl)propan-2-yl)phenyl)picolinamide (204)

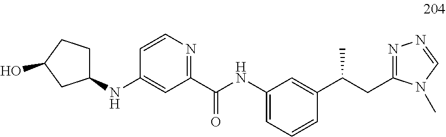 [SMILES: C[C@H](Cc1nncn1C)c1cccc(NC(=O)c2cc(N[C@@H]3CC[C@H](O)C3)ccn2)c1]

Compound 204 (18 mg, 2%) was obtained according to procedures disclosed herein as an off-white solid. ¹H NMR (300 MHz, DMSO-d₆) δ 8.27 (s, 2H), 8.14 (d, J=6.0 Hz, 1H), 7.77-7.75 (m, 2H), 7.31-7.22 (m, 2H), 7.04-6.96 (m, 2H), 6.68-6.65 (m, 1H), 4.54 (s, 1H), 4.16-4.12 (m, 1H), 3.88-3.77 (m, 1H), 3.44 (s, 3H), 3.26-3.19 (m, 1H), 2.96 (d, J=7.5 Hz, 1H), 2.31-2.27 (m, 1H), 2.00-1.97 (m, 1H), 1.73-1.59 (m, 3H), 1.42-1.37 (m, 1H), 1.27 (d, J=6.9 Hz, 3H). MS (ESI) calculated for (C₂₃H₂₈N₆O₂) [M+H]⁺, 421; found, 421.

### Example 205: trans-4-(3-hydroxycyclopentyloxy)-N-(3-((R)-1-(4-methyl-4H-1,2,4-triazol-3-yl)propan-2-yl)phenyl)picolinamide (205)

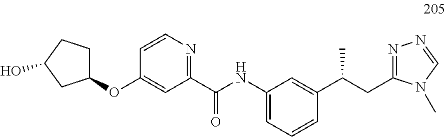 [SMILES: C[C@H](Cc1nncn1C)c1cccc(NC(=O)c2cc(O[C@@H]3CC[C@@H](O)C3)ccn2)c1]

Step 1: trans-methyl 4-(3-hydroxycyclopentyloxy)picolinate. To a solution of methyl 4-hydroxypicolinate (800 mg, 5.22 mmol) in THF (10 mL) were added cis-cyclopentane-1,3-diol (533 mg, 5.22 mmol), triphenylphosphine (2.7 g, 10.45 mmol) and diisopropyl azodicarboxylate (2.1 g, 10.44 mmol). The mixture was stirred at rt for 16 h. The mixture was diluted with water, followed by General Work-up Procedure 1. The residue was purified by reverse phase flash column chromatography with 5-100% acetonitrile in water to the title compound (380 mg, 31%) as a yellow oil.

Step 2: trans-4-(3-hydroxycyclopentyloxy)picolinic acid. To a solution of trans-methyl 4-(3-hydroxycyclopentyloxy)picolinate (380 mg, 1.60 mmol) in THF/water (10/5 mL) was added lithium hydroxide (77 mg, 3.21 mmol). The mixture was stirred at rt for 4 h. The mixture was diluted with water and the pH was adjusted to 6 with 1 N hydrochloric acid and then evaporated in vacuo to afford the title compound (400 mg, crude) as a yellow solid, which was used without purification.

Step 3: trans-4-(3-hydroxycyclopentyloxy)-N-(3-((R)-1-(4-methyl-4H-1,2,4-triazol-3-yl)propan-2-yl)phenyl)picolinamide. Followed General procedure 6 to afford compound 205 (54.2 mg, 14%) as a light yellow solid. 1H NMR (300 MHz, DMSO-d₆) δ 10.51 (s, 1H), 8.51 (d, J=5.7 Hz, 1H), 8.28 (s, 1H), 7.81-7.77 (m, 2H), 7.59 (d, J=2.4 Hz, 1H), 7.29 (s, 1H), 7.19-7.17 (m, 1H), 6.99 (d, J=7.8 Hz, 1H), 5.13-5.11 (m, 1H), 4.70 (d, J=3.6 Hz, 1H), 4.30 (d, J=3.6 Hz, 1H), 3.45 (s, 3H), 3.25-3.21 (m, 1H), 2.97 (d, J=7.2 Hz, 2H), 2.28-2.22 (m, 1H), 2.04-2.01 (m, 1H), 1.96-1.85 (m, 2H), 1.72-1.71 (m, 1H), 1.60-1.57 (m, 1H) 1.28 (d, J=6.9 Hz, 3H). MS (ESI) calculated for (C₂₃H₂₇N₅O₃) [M+H]⁺, 422; found, 422.

### Example 206 and Example 207: 4-((1S,3R)-3-hydroxycyclopentyloxy)-N-(3-((R)-1-(4-methyl-4H-1,2,4-triazol-3-yl)propan-2-yl)phenyl)picolinamide (206) and 4-((1R,3S)-3-hydroxycyclopentyloxy)-N-(3-((R)-1-(4-methyl-4H-1,2,4-triazol-3-yl)propan-2-yl)phenyl)picolinamide (207)

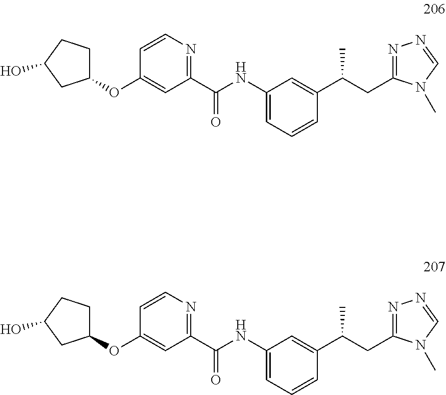 [SMILES: C[C@H](Cc1nncn1C)c1cccc(NC(=O)c2cc(O[C@H]3CC[C@@H](O)C3)ccn2)c1 ; C[C@H](Cc1nncn1C)c1cccc(NC(=O)c2cc(O[C@@H]3CC[C@@H](O)C3)ccn2)c1]

Step 1: cis-methyl 4-(3-hydroxycyclopentyloxy)picolinate. To a solution of methyl 4-hydroxypicolinate (2.3 g, 5.0 mmol) in THF (30 mL) were added trans-cyclopentane-1,3-diol (1.0 g, 9.7 mmol), triphenylphosphine (5.1 g, 19.5 mmol) and diisopropyl azodicarboxylate (4.0 g, 19.5 mmol). The mixture was stirred at rt for 16 h. The mixture was diluted with water, followed by General Work-up Procedure 1. The residue was purified by reverse phase flash column chromatography with 5-100% acetonitrile in water to afford the title compound (400 mg, 17%) as a yellow oil.

Step 2: Synthesis of cis-4-(3-hydroxycyclopentyloxy)picolinic acid. To a solution of cis-methyl 4-(3-hydroxycyclopentyloxy)picolinate (380 mg, 1.60 mmol) in THF/water (10/5 mL) was added lithium hydroxide (77 mg, 3.21 mmol). The mixture was stirred at rt for 4 h. The mixture was diluted with water (100 mL). The pH value of the mixture was adjusted to 6 with hydrochloric acid (1M) and then evaporated in vacuo to afford the title compound (450 mg, crude) as a yellow solid.

Step 3: Synthesis of cis-4-(3-hydroxycyclopentyloxy)-N-(3-((K)-1-(4-methyl-4H-1,2,4-triazol-3-yl)propan-2-yl)phenyl)picolinamide. Followed General procedure 6 to afford the title compound (140 mg, 25%) as a colorless solid.

Step 4: Synthesis of 206 and 207. The enantiomers were separated using chiralpak IF column with CO₂ and methanol as mobile phase to afford 206 (26 mg) as a colorless solid and 207 (34 mg) as a colorless solid.

206: ¹H NMR (300 MHz, DMSO-d₆) δ 10.51 (s, 1H), 8.51 (d, J=5.4 Hz, 1H), 8.28 (s, 1H), 7.82-7.77 (m, 2H), 7.60 (d, J=1.8 Hz, 1H), 7.29-7.24 (m, 1H), 7.19-7.16 (m, 1H), 7.00 (d, J=7.8 Hz, 1H), 4.98-4.95 (m, 1H), 4.71 (d, J=4.2 Hz, 1H), 4.16 (d, J=5.4 Hz, 1H), 3.45 (s, 3H), 3.25-3.21 (m, 1H), 2.97 (d, J=7.5 Hz, 2H), 2.49-2.37 (m, 1H), 2.05-1.91 (m, 1H), 1.79-1.58 (m, 4H), 1.28 (d, J=6.9 Hz, 3H). MS (ESI) calculated for (C₂₃H₂₇N₅O₃) [M+H]⁺, 422; found, 422.

207: ¹H NMR (300 MHz, DMSO-d₆) δ 10.51 (s, 1H), 8.51 (d, J=5.4 Hz, 1H), 8.28 (s, 1H), 7.82-7.76 (m, 2H), 7.60 (d, J=2.7 Hz, 1H), 7.29-7.24 (m, 1H), 7.19-7.16 (m, 1H), 7.00 (d, J=7.8 Hz, 1H), 4.98-4.95 (m, 1H), 4.70 (d, J=4.2 Hz, 1H), 4.15 (d, J=5.7 Hz, 1H), 3.45 (s, 3H), 3.25-3.20 (m, 1H), 2.97 (d, J=7.8 Hz, 2H), 2.51-2.37 (m, 1H), 2.05-2.00 (m, 1H), 1.79-1.57 (m, 4H), 1.28 (d, J=6.6 Hz, 3H). MS (ESI) calculated for (C₂₃H₂₇N₅O₃) [M+H]⁺, 422; found, 422.

### Example 208: cis-4-(3-hydroxycyclopentylamino)-N-(3-((R)-1-(4-methyl-4H-1,2,4-triazol-3-yl)propan-2-yl)phenyl)picolinamide (208)

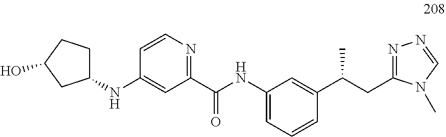 [SMILES: C[C@H](Cc1nncn1C)c1cccc(NC(=O)c2cc(N[C@H]3CC[C@@H](O)C3)ccn2)c1]

Followed Example 204, step 2 to afford the title compound (2 mg, 0.2%) as an off-white solid. ¹H NMR (300 MHz, DMSO-d₆) δ 10.39 (s, 1H), 8.29 (s, 1H), 8.14 (s, 1H), 7.77 (s, 2H), 7.30-7.22 (m, 2H), 7.06-6.67 (m, 2H), 6.67 (s, 1H), 4.67 (d, J=3.6 Hz, 1H), 4.15-4.12 (m, 1H), 3.79-3.71 (m, 1H), 3.45 (s, 3H), 3.24-3.22 (m, 1H), 2.97 (d, J=7.2 Hz, 2H), 2.33-2.24 (m, 1H), 2.00-1.97 (m, 1H), 1.75-1.59 (m, 3H), 1.41-1.37 (m, 1H), 1.27 (d, J=6.9 Hz, 3H). MS (ESI) calculated for (C₂₃H₂₈N₆O₂) [M+H]⁺, 421; found, 421.

### Example 209: (R)-6-(4-methyl-3-oxopiperazin-1-yl)-N-(3-(1-(4-methyl-4H-1,2,4-triazol-3-yl)propan-2-yl)phenyl)isoquinoline-3-carboxamide 2,2,2-trifluoroacetate (209)

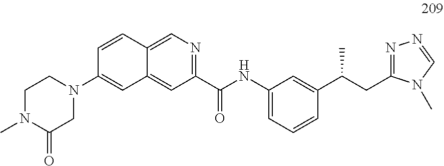 [SMILES: C[C@H](Cc1nncn1C)c1cccc(NC(=O)c2cc3cc(N4CCN(C)C(=O)C4)ccc3cn2)c1]

Step 1: methyl 6-bromoisoquinoline-3-carboxylate. To a solution of 6-bromoisoquinoline-3-carboxylic acid (0.67 g, 2.7 mmol) in 30 mL of methanol was added 1-2 mL of concentrated sulfuric acid and stirred at 60-70° C. for 24 h. The cooled reaction mixture was concentrated, partitioned between water and EtOAc, the organic layer was washed with aqueous sodium bicarbonate, dried, filtered, concentrated and dried to give titled compound (0.42 g 59%).

Step 2: Methyl 6-(4-methyl-3-oxo-piperazin-1-yl)isoquinoline-3-carboxylate. To a vial were added methyl 6-bromoisoquinoline-3-carboxylate (83 mg, 0.31 mmol), 1-methylpiperazin-2-one (85 mg, 0.74 mmol), potassium phosphate, tribasic (175 mg, 0.81 mmol) and dioxane (2.5 mL). The mixture was degassed by sparging with argon, RuPhos palladacycle Gen 4 pre-catalyst (22 mg, 8 mol %) was added and the vial was sealed and stirred at 100° C. overnight. The reaction mixture was filtered, collected solids washed with EtOAc and purified on SiO₂ (0-10% methanol/dichloromethane) to give titled compound titled compound (20 mg 16%) as a yellow solid.

Step 3: Lithium 6-(4-methyl-3-oxopiperazin-1-yl)isoquinoline-3-carboxylate. To a warm solution of methyl 6-(4-methyl-3-oxopiperazin-1-yl)isoquinoline-3-carboxylate (20 mg, 0.067 mmol) in 1 mL of THF and 1 mL of methanol was added a solution of lithium hydroxide monohydrate (4.5 mg, 0.10 mmol). The mixture was stirred at rt for 2-3 h, then concentrated to dryness to the title compound as an off-white solid which was used without purification.

Step 4: 209. Followed General procedure 6 to give titled compound (50 mg 20%) as a light-yellow solid. 1H NMR (500 MHz, Methanol-d₄) δ 9.14 (s, 1H), 8.79 (s, 1H), 8.53 (s, 1H), 8.17 (d, J=9.2 Hz, 1H), 7.73 (t, J=1.9 Hz, 1H), 7.67 (dd, J=9.3, 2.5 Hz, 1H), 7.61 (ddd, J=8.0, 2.2, 1.0 Hz, 1H), 7.35 (t, J=7.9 Hz, 1H), 7.32 (d, J=2.5 Hz, 1H), 7.07 (dt, J=7.7, 1.3 Hz, 1H), 4.17 (s, 2H), 3.88 (dd, J=6.4, 4.5 Hz, 2H), 3.68-3.56 (m, 5H), 3.42-3.33 (m, 2H), 3.27-3.22 (m, 1H), 3.07 (s, 3H), 1.48 (d, J=6.8 Hz, 3H). LCMS: C₂₇H₂₉N₇O₂ requires: 483, found: m/z=484 [M+H]⁺

### Example 210: (S)-6-(4-methyl-3-oxopiperazin-1-yl)-N-(3-(1-(4-methyl-4H-1,2,4-triazol-3-yl)propan-2-yl)phenyl)isoquinoline-3-carboxamide 2,2,2-trifluoroacetate (210)

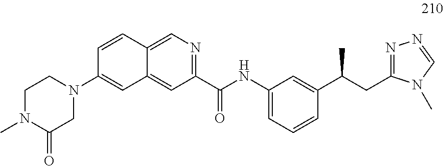 [SMILES: C[C@@H](Cc1nncn1C)c1cccc(NC(=O)c2cc3cc(N4CCN(C)C(=O)C4)ccc3cn2)c1]

The title compound (18 mg 42%) was prepared in a similar manner as Example 209. ¹H NMR (500 MHz, Methanol-d₄) δ 9.16 (s, 1H), 8.87 (s, 1H), 8.56 (s, 1H), 8.21 (d, J=9.3 Hz, 1H), 7.72 (t, J=2.0 Hz, 1H), 7.70 (dd, J=9.3, 2.5 Hz, 1H), 7.61 (ddd, J=8.1, 2.1, 0.9 Hz, 1H), 7.36 (t, J=7.9 Hz, 1H), 7.33 (d, J=2.5 Hz, 1H), 7.09 (dt, J=7.7, 1.4 Hz, 1H), 4.20 (s, 2H), 3.97-3.86 (m, 2H), 3.68-3.59 (m, 5H), 3.49-3.22 (m, 3H), 3.07 (s, 3H), 1.49 (d, J=6.8 Hz, 3H). LCMS:C27H29N7O2 requires: 483, found: m/z=484 [M+H]⁺

### Example 211: (R)-6-(4-acetylpiperazin-1-yl)-N-(3-(1-(4-methyl-4H-1,2,4-triazol-3-yl)propan-2-yl)phenyl)isoquinoline-3-carboxamide 2,2,2-trifluoroacetate (211)

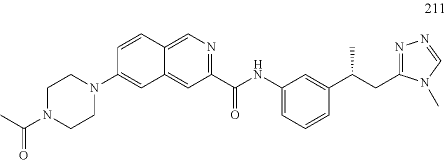 [SMILES: CC(=O)N1CCN(c2ccc3cnc(C(=O)Nc4cccc([C@H](C)Cc5nncn5C)c4)cc3c2)CC1]

Step 1: Methyl 6-(4-acetylpiperazin-1-yl)isoquinoline-3-carboxylate. To a solution of methyl 6-bromoisoquinoline-3-carboxylate (117 mg, 0.44 mmol), 1-acetylpiperazine (64 mg, 0.49 mmol) in dioxane (2 mL) was added potassium phosphate tribasic (285 mg, 3.0 mmol). The mixture was degassed with argon, then added RuPhos palladacycle Gen 4 pre-catalyst (35 mg, 9 mol %) and the vial sealed and heated to 80° C. overnight. The cooled reaction mixture was diluted with EtOAc, filtered through Celite™, washed solids with EtOAc, concentrated filtrate and purified on SiO₂ (0-5% methanol/dichloromethane) to give the title compound (125 mg, 90%) as a yellow solid.

Step 2: Lithium 6-(4-methyl-3-oxopiperazin-1-yl)isoquinoline-3-carboxylate. Followed Example 209, step 3.

Step 3: (R)-6-(4-acetylpiperazin-1-yl)-N-(3-(1-(4-methyl-4H-1,2,4-triazol-3-yl)propan-2-yl)phenyl)isoquinoline-3-carboxamide 2,2,2-trifluoroacetate. Followed Example 197 to give titled compound (29 mg, 44%). ¹H NMR (500 MHz, Methanol-d₄) δ 9.14 (s, 1H), 8.89 (s, 1H), 8.56 (s, 1H), 8.20 (d, J=9.3 Hz, 1H), 7.79-7.69 (m, 2H), 7.61 (dd, J=7.8, 2.0 Hz, 1H), 7.42-7.28 (m, 2H), 7.12-7.05 (m, 1H), 3.81 (dq, J=8.4, 3.6, 2.6 Hz, 4H), 3.73 (dd, J=6.8, 3.6 Hz, 2H), 3.68 (dd, J=6.7, 4.1 Hz, 2H), 3.65 (s, 3H), 3.44-3.26 (m, 3H), 2.17 (s, 3H), 1.49 (d, J=6.7 Hz, 3H). LCMS: C₂₈H₃₁N₇O₂ requires: 497, found: m/z=498 [M+H]⁺

### Example 212: (R)-N-(3-(1-(4H-1,2,4-triazol-3-yl)propan-2-yl)phenyl)-6-cyclopropyl-5-methylpicolinamide (212)

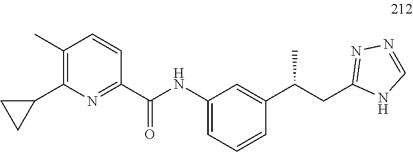 [SMILES: Cc1ccc(C(=O)Nc2cccc([C@H](C)Cc3nnc[nH]3)c2)nc1C1CC1]

Followed General procedure 6 using (R)-3-(1-(4H-1,2,4-triazol-3-yl)propan-2-yl)aniline to afford compound 212 (27 mg, 13%) as a colorless solid. ¹H NMR (400 MHz, Chloroform-d) δ 9.92 (s, 1H), 7.96-7.90 (m, 2H), 7.81 (t, J=2.0 Hz, 1H), 7.60 (d, J=7.6 Hz, 1H), 7.35-7.25 (m, 2H), 6.94-6.93 (m, 1H), 3.36-3.31 (m, 1H), 3.29-3.14 (m, 2H), 2.53 (s, 3H), 2.23-2.16 (m, 1H), 1.38 (d, J=6.8 Hz, 3H), 1.17-1.04 (m, 4H). MS (ESI) calc'd for (C₂₁H₂₃N₅O) [M+H]⁺, 362; found, 362.

### Example 213: (R)-N-(3-(1-(4-methylisoxazol-3-yl)propan-2-yl)phenyl)-2-(trifluoromethyl)pyrimidine-4-carboxamide (213a)

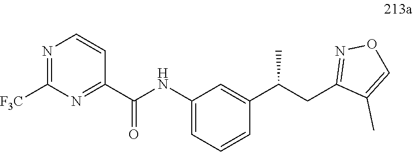 [SMILES: Cc1conc1C[C@@H](C)c1cccc(NC(=O)c2ccnc(C(F)(F)F)n2)c1]

The amide bond formation reaction was carried out in a similar fashion as for 74 using (R)-3-(1-(4-methylisoxazol-3-yl)propan-2-yl)aniline (50 mg, 0.23) mmol and 2-(trifluoromethyl)pyrimidine-4-carboxylic acid (53 mg, 0.277 mmol) to afforded the title compound (82 mg, 90%) as a yellow oil. 1H NMR (500 MHz, DMSO-d6) δ 10.60 (s, 1H), 9.36 (d, J=5.0 Hz, 1H), 8.50 (d, J=1.3 Hz, 1H), 8.37 (d, J=5.0 Hz, 1H), 7.82-7.67 (m, 2H), 7.32 (t, J=7.8 Hz, 1H), 7.09 (dt, J=7.7, 1.3 Hz, 1H), 3.15 (h, J=6.9 Hz, 1H), 2.95-2.82 (m, 2H), 1.86 (d, J=1.1 Hz, 3H), 1.27 (d, J=6.9 Hz, 3H). LCMS: C₁₉H₁₇F₃N₄O₂ requires: 390, found: m/z=391 [M+H]⁺.

### (S)-N-(3-(1-(4-methylisoxazol-3-yl)propan-2-yl)phenyl)-2-(trifluoromethyl)pyrimidine-4-carboxamide (213b)

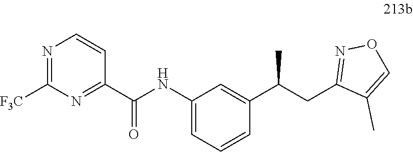 [SMILES: Cc1conc1C[C@H](C)c1cccc(NC(=O)c2ccnc(C(F)(F)F)n2)c1]

The amide bond formation reaction was carried out in a similar fashion as for 74 using (S)-3-(1-(4-methylisoxazol-3-yl)propan-2-yl)aniline (50 mg, 0.23) mmol and 2-(trifluoromethyl)pyrimidine-4-carboxylic acid (53 mg, 0.277 mmol) to afforded the title compound (106 mg, 118%, product contained acetonitrile and water) as a yellow oil. 1H NMR (500 MHz, DMSO-d6) δ 10.60 (s, 1H), 9.36 (d, J=5.0 Hz, 1H), 8.50 (d, J=1.2 Hz, 1H), 8.37 (d, J=5.0 Hz, 1H), 7.82-7.67 (m, 2H), 7.32 (t, J=7.8 Hz, 1H), 7.09 (dt, J=7.7, 1.3 Hz, 1H), 3.15 (h, J=6.8 Hz, 1H), 2.95-2.82 (m, 2H), 1.86 (d, J=1.1 Hz, 3H), 1.27 (d, J=7.0 Hz, 3H). LCMS: C₁₉H₁₇F₃N₄O₂ requires: 390, found: m/z=391 [M+H]⁺.

### Example 214: 6-Cyclopropyl-5-methyl-N-(3-(1-(1-methyl-1H-imidazol-2-yl)propan-2-yl)phenyl)picolinamide (214)

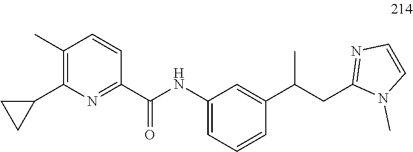 [SMILES: Cc1ccc(C(=O)Nc2cccc(C(C)Cc3nccn3C)c2)nc1C1CC1]

Followed General procedure 1-G using H-1 to afford 214 (150 mg, 74%) as a yellow oil. ¹H NMR (400 MHz, DMSO-d₆) δ 8.26 (s, 1H), 7.77-7.65 (m, 2H), 7.50-7.48 (m, 2H), 7.28 (t, J=8.4 Hz, 1H), 7.16-7.15 (m, 2H), 6.97 (d, J=7.6 Hz, 1H), 3.46 (s, 3H), 3.29-2.96 (m, 3H), 2.42 (s, 3H), 2.23-2.19 (m, 1H), 1.27 (d, J=6.8 Hz, 3H), 1.10-1.01 (m, 4H). MS (ESI) calc'd for (C₂₃H₂₆N₄O) [M+H]⁺, 375; found, 375.

### Example 215 and Example 216: N-[3-[1-methyl-2-(5-methylpyrazol-1-yl)ethyl]phenyl]-6-(trifluoromethyl)pyridine-2-carboxamide (215) and N-(3-(1-(3-methyl-1H-pyrazol-1-yl)propan-2-yl)phenyl)-6-(trifluoromethyl)picolinamide (216)

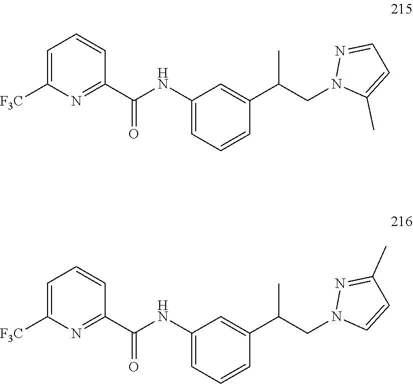 [SMILES: Cc1ccnn1CC(C)c1cccc(NC(=O)c2cccc(C(F)(F)F)n2)c1 ; Cc1ccn(CC(C)c2cccc(NC(=O)c3cccc(C(F)(F)F)n3)c2)n1]

Step 1: N-(3-acetylphenyl)-6-(trifluoromethyl)picolinamide. To a solution of 6-(trifluoromethyl)picolinic acid (4.08 g, 20.7 mmol) in 25 mL of N,N-di methylformamide was added HATU (8.7 g, 21.8 mmol) and N,N-diisopropylethylamine (8.5 mL, 49 mmol). The mixture turned yellow in color and was continued to stir for 10-15 min, then 3′-aminoacetophenone (3.0 g, 21.8 mmol) was added and stirred at rt under a nitrogen atmosphere overnight. The mixture was partitioned between water and EtOAc, the separated organic layer was washed with water, dried, filtered through a pad of silica gel, concentrated, then purified on SiO₂ (20-70% EtOAc/Hexanes) to give the title compound (6.2, 96%) as an off-white solid.

Step 2: N-(3-(2-bromoacetyl)phenyl)-6-(trifluoromethyl)picolinamide. To a rt solution of N-(3-acetylphenyl)-6-(trifluoromethyl)pyridine-2-carboxamide (5.1 g, 16.5 mmol) in 80 mL of chloroform was slowly added bromine (0.52 g, 3.2 mmol) in 10 mL of chloroform, The mixture was stirred at rt. The resulting suspension was filtered and collected solids washed with dichloromethane and dried to give the titled compound (1.32 g). The mother liquor was diluted with dichloromethane, washed with dilute aqueous sodium bicarbonate, dried, filtered and concentrated until a suspension formed. The solid was filtered and washed with dichloromethane/hexanes and dried to give the titled compound (2.25 g), giving a combined amount of the title compound (3.6, 56%) as a colorless solid.

Step 3: N-[3-[2-(5-methylpyrazol-1-yl)acetyl]phenyl]-6-(trifluoromethyl)pyridine-2-carboxamide. To a solution of N-[3-(2-bromoacetyl)phenyl]-6-(trifluoromethyl)pyridine-2-carboxamide (0.27 g, 0.70 mmol) in 4 mL of N,N-dimethylformamide was added potassium carbonate (0.32 g, 2.3 mmol) and a solution of 3-methyl-1H-pyrazole (0.082 g, 0.99 mmol). The mixture was heated to 60° C. for 2-3 h then cooled to rt and stirred for an additional 2 days. The resulting o mixture was filtered, partitioned between water, neutralized with dilute aqueous hydrochloric acid, followed by General Work-up Procedure 1. The crude yellow material was twice purified on SiO₂ (20-50% EtOAc/hexanes) to give a mixture of N-[3-[2-(5-methylpyrazol-1-yl)acetyl]phenyl]-6-(trifluoromethyl)pyridine-2-carboxamide and N-[3-[2-(3-methylpyrazol-1-yl)acetyl]phenyl]-6-(trifluoromethyl)pyridine-2-carboxamide (60 mg, 22%).

Step 4: Synthesis of N-(3-(3-(5-methyl-1H-pyrazol-1-yl)prop-1-en-2-yl)phenyl)-6-(trifluoromethyl)picolinamide/N-(3-(3-(3-methyl-1H-pyrazol-1-yl)prop-1-en-2-yl)phenyl)-6-(trifluoromethyl)picolinamide. To a 0° C. suspension of potassium tert-butoxide (74 mg, 0.65 mmol) in 3 mL of methyl-THF was added methyl triphenyl phosphonium bromide (220 mg, 0.61 mmol) under an argon atmosphere. The suspension was stirred in an ice bath for 40-45 min, then slowly a solution of Step 3 (60 mg, 0.15 mmol) was added in 3 mL of 2-Methyltetrahydrofuran. The resulting suspension slowly warmed to rt and stirred overnight. The mixture was partitioned between EtOAc and water, dried, filtered and concentrated to give a crude red liquid which was purified on SiO₂ (20-50% EtOAc/hexanes) to give a mixture of N-(3-(3-(5-methyl-1H-pyrazol-1-yl)prop-1-en-2-yl)phenyl)-6-(trifluoromethyl)picolinamide and N-(3-(3-(3-methyl-1H-pyrazol-1-yl)prop-1-en-2-yl)phenyl)-6-(trifluoromethyl)picolinamide (16 mg, 27%)

Step 5: N-(3-(1-(5-methyl-1H-pyrazol-1-yl)propan-2-yl)phenyl)-6-(trifluoromethyl)picolinamide and N-(3-(1-(3-methyl-1H-pyrazol-1-yl)propan-2-yl)phenyl)-6-(trifluoromethyl)picolinamide. To a vial was added a mixture of N-(3-(3-(5-methyl-1H-pyrazol-1-yl)prop-1-en-2-yl)phenyl)-6-(trifluoromethyl)picolinamide and N-(3-(3-(3-methyl-1H-pyrazol-1-yl)prop-1-en-2-yl)phenyl)-6-(trifluoromethyl)picolinamide (16 mg, 0.041 mmol), palladium on carbon (23 mg, 10% wet, ˜50%) and 2 mL of ethanol. The vial was degassed with argon then charged with hydrogen and stirred under 1 atm of hydrogen for 2-3 h. The mixture was filtered through a pad of Celite™, concentrated and purified on silica gel followed by lyophilization in acetonitrile/water (1:1 v/v) to afford the title compound (12 mg, 76%) as a colorless amorphous film. The isomers were separated by SFC to give:

N-(3-(1-(5-methyl-1H-pyrazol-1-yl)propan-2-yl)phenyl)-6-(trifluoromethyl)picolinamide as a colorless amorphous solid. Yield: 6.8 mg (43%). ¹H NMR (500 MHz, Methanol-d₄) δ 8.45 (d, J=7.9 Hz, 1H), 8.28 (t, J=7.8 Hz, 1H), 8.04 (dd, J=7.8, 1.0 Hz, 1H), 7.69 (ddd, J=8.1, 2.2, 1.0 Hz, 1H), 7.53 (t, J=1.9 Hz, 1H), 7.36 (d, J=1.8 Hz, 1H), 7.27 (t, J=7.9 Hz, 1H), 6.91 (dt, J=7.7, 1.3 Hz, 1H), 5.92 (dd, J=1.9, 0.9 Hz, 1H), 4.17 (dd, J=12.9, 7.6 Hz, 2H), 3.37 (m, 1H), 1.96 (s, 3H), 1.33 (d, J=7.1 Hz, 3H). LCMS: C₂₀H₁₉F₃N₄O requires: 388, found: m/z=389 [M+H]⁺

N-(3-(1-(3-methyl-1H-pyrazol-1-yl)propan-2-yl)phenyl)-6-(trifluoromethyl)picolinamide as a colorless film. Yield: 0.9 mg (5.6%). ¹H NMR (500 MHz, Methanol-d₄) δ 8.46 (d, J=7.9 Hz, 1H), 8.29 (td, J=7.9, 0.8 Hz, 1H), 8.05 (dd, J=7.8, 1.0 Hz, 1H), 7.69 (ddd, J=8.1, 2.1, 1.0 Hz, 1H), 7.60 (t, J=1.9 Hz, 1H), 7.30 (t, J=7.9 Hz, 1H), 7.24 (d, J=2.2 Hz, 1H), 7.00 (dt, J=7.6, 1.4 Hz, 1H), 5.92 (d, J=2.1 Hz, 1H), 4.20 (dd, J=7.7, 1.9 Hz, 2H), 3.35 (m, 1H), 2.22 (s, 3H), 1.25 (d, J=7.0 Hz, 3H). LCMS: C₂₀H₁₉F₃N₄O requires: 388, found: m/z=389 [M+H]⁺

### Example 217: N-[3-[1-methyl-2-(5-methyltetrazol-1-yl)ethyl]phenyl]-6-(trifluoromethyl)pyridine-2-carboxamide (217)

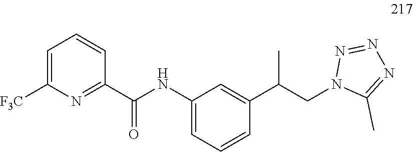 [SMILES: Cc1nnnn1CC(C)c1cccc(NC(=O)c2cccc(C(F)(F)F)n2)c1]

Prepared in a similar manner to 215./216 from N-[3-(2-bromoacetyl)phenyl]-6-(trifluoromethyl)pyridine-2-carboxamide using 5-methyl-1H-tetrazoleto give the title compound (38 mg, 33% two steps). ¹H NMR (500 MHz, CDCl₃) δ 9.77 (s, 1H), 8.50 (d, J=7.8 Hz, 1H), 8.19-8.09 (m, 1H), 7.90 (dd, J=7.8, 1.1 Hz, 1H), 7.79 (t, J=2.0 Hz, 1H), 7.58-7.49 (m, 1H), 7.29 (t, J=7.9 Hz, 1H), 6.77 (dt, J=7.6, 1.3 Hz, 1H), 4.51 (dd, J=13.8, 6.2 Hz, 1H), 4.29 (dd, J=13.8, 8.5 Hz, 1H), 3.55-3.42 (m, 1H), 2.12 (s, 3H), 1.46 (d, J=7.0 Hz, 3H). LCMS: C₁₈H₁₇F₃N₆O requires: 390, found: m/z=391 [M+H]⁺

### Example 218: N-[3-[1-methyl-2-(5-methyltriazol-1-yl)ethyl]phenyl]-6-(trifluoromethyl)pyridine-2-carboxamide (218)

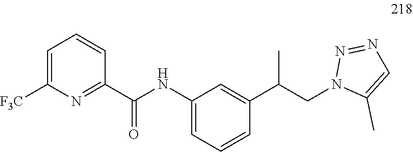 [SMILES: Cc1cnnn1CC(C)c1cccc(NC(=O)c2cccc(C(F)(F)F)n2)c1]

Step 1: 2-(3-nitrophenyl)propyl methanesulfonate. To a 0° C. solution of 2-(3-nitrophenyl)propan-1-ol (WO2005014552) (0.18 g, 0.99 mmol) in 4 mL of THF was added methane sulfonyl chloride (0.10 mL, 1.3 mmol) and triethylamine (0.15 mL, 1.1 mmol) dropwise under an nitrogen atmosphere which immediately formed a white suspension. The mixture was warmed to rt, stirred for 2 h then partitioned between water and EtOAc, the organic layer was washed with brine, dried, filtered and concentrated to the title compound as a light-brown liquid which was used without purification.

Step 2: 1-(2-azido-1-methyl-ethyl)-3-nitro-benzene. To a solution of 2-(3-nitrophenyl)propyl methanesulfonate (0.25 g, 0.96 mmol) in 3 mL of N, N-dimethylformamide was added sodium azide (0.12 mg, 1.8 mmol) which was heated to 80° C. for 4-5 h. The cooled reaction mixture was partitioned between EtOAc and water, dried, filtered and purified by flash column chromatography (0-50% EtOAc/hexanes) to give the title compound (140 mg, 72%).

Step 3: 3-[1-methyl-2-(5-methyltriazol-1-yl)ethyl]aniline. To a solution of 1-(2-azido-1-methyl-ethyl)-3-nitro-benzene (0.10 g, 0.52 mmol) in 1 mL of dioxanes was added 1-dimethoxyphosphorylpropan-2-one (72 μL, 0.52 mmol) and crushed potassium hydroxide pellets (0.11 g, 2.0 mmol). The mixture thickened within minutes as heating was applied, so an additional amount of dioxanes (1 mL) was added and stirred at 60° C. for 18 h. The cooled reaction mixture was partitioned between water and EtOAc, and the separated organic layer was dried, filtered, concentrated was purified on silica gel (50-100% EtOAc/hexanes) to give the title compound (58 mg, 48%) as a pale-yellow liquid.

Step 4: 3-[1-methyl-2-(5-methyltriazol-1-yl)ethyl]aniline. To a vial was added 10% palladium on carbon (33 mg, 0.031 mmol, 10 mol %), and a solution of 5-methyl-1-[2-(3-nitrophenyl)propyl]triazole (74 mg, 0.30 mmol) in ethanol (4 mL). The mixture was degassed with nitrogen and then charged with hydrogen. The mixture was stirred under 1 atmosphere of hydrogen for 6 h. The mixture was filtered through a pad of Celite™, and concentrated to the title compound as a colorless film which was used without purification.

Step 5: N-[3-[1-methyl-2-(5-methyltriazol-1-yl)ethyl]phenyl]-6-(trifluoromethyl)pyridine-2-carboxamide. Followed General procedure 1-G to provide the title compound (72 mg, 84%) as an off-white solid. ¹H NMR (500 MHz, DMSO-d₆) δ 10.32 (s, 1H), 8.53-8.32 (m, 2H), 8.18 (dd, J=7.4, 1.4 Hz, 1H), 7.78 (ddd, J=8.2, 2.2, 1.0 Hz, 1H), 7.70 (t, J=1.9 Hz, 1H), 7.38 (d, J=1.0 Hz, 1H), 7.29 (t, J=7.9 Hz, 1H), 6.98 (dt, J=7.6, 1.4 Hz, 1H), 4.42 (qd, J=13.9, 7.5 Hz, 2H), 3.34 (q, J=7.2 Hz, 1H), 2.07 (d, J=0.8 Hz, 3H), 1.27 (d, J=7.0 Hz, 3H). LCMS: C₁₉H₁₈F₃N₅O requires: 389, found: m/z=390 [M+H]⁺.

### Example 219: N-[3-[1-methyl-2-(3-oxo-1H-1,2,4-triazol-2-yl)ethyl]phenyl]-6-(trifluoromethyl)pyridine-2-carboxamide (219)

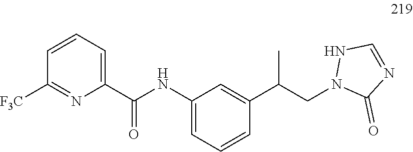 [SMILES: CC(Cn1[nH]cnc1=O)c1cccc(NC(=O)c2cccc(C(F)(F)F)n2)c1]

Compound 219 (21.6 mg, yield 33%) was obtained according to procedures disclosed herein. ¹H NMR (500 MHz, Acetone-d6) δ 10.26 (s, 1H), 8.53 (d, J=7.8 Hz, 1H), 8.45-8.38 (m, 1H), 8.14 (dd, J=7.8, 1.0 Hz, 1H), 7.88 (ddd, J=8.1, 2.1, 1.0 Hz, 1H), 7.82 (t, J=1.9 Hz, 1H), 7.45 (s, 1H), 7.36 (t, J=7.9 Hz, 1H), 7.10 (dt, J=7.6, 1.4 Hz, 1H), 3.90-3.78 (m, 2H), 3.35-3.27 (m, 1H), 1.33 (d, J=7.0 Hz, 3H). LCMS: C₁₈H₁₆F₃N₅O₂ requires: 391, found: m/z, 392 [M+H]⁺.

### Example 220: 4-((S)-3-hydroxypyrrolidine-1-carbonyl)-N-(3-((R-1-(4-methyl-4H-1,2,4-triazol-3-yl)propan-2-yl)phenyl)-6-(trifluoromethyl)picolinamide (220)

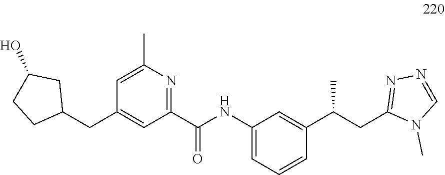 [SMILES: Cc1cc(CC2CC[C@H](O)C2)cc(C(=O)Nc2cccc([C@H](C)Cc3nncn3C)c2)n1]

Step 1: Methyl 6-(trifluoromethyl)-4-vinylpicolinate. A solution of vinylboronic anhydride pyridine complex (1.57 g, 6.51 mmol), methyl 4-chloro-6-(trifluoromethyl)pyridine-2-carboxylate (1.30 g, 5.43 mmol), tris(dibenzylideneacetone)dipalladium(0) (0.248 g, 0.271 mmol), potassium carbonate (3.04 g, 21.7 mmol) and 2-dicyclohexylphosphino-2′,6′-dimethoxybiphenyl (0.469 g, 1.09 mmol) in dioxane/water (10:1 v/v, 13.6 mL) was evacuated and back filled with nitrogen. The reaction was stirred for 1 h at 60° C. The mixture was diluted with water and EtOAc. The phases were separated and the aqueous layer was extracted with EtOAc 2×. Purification by flash column chromatography eluting with 0-25% EtOAc/hexanes to give the title compound as an off-white solid. Yield: 0.736 g (58.7%).

Step 2: 2-(Methoxycarbonyl)-6-(trifluoromethyl)isonicotinic acid. A solution of methyl 6-(trifluoromethyl)-4-vinylpicolinate (0.736 g, 3.19 mmol) and sodium periodate (2.74 g, 12.7 mmol) in acetonitrile/water/chloroform (1:1:1 v/v/v, 15.9 mL) was treated with ruthenium(III) chloride hydrate (0.0341 g, 0.159 mmol) at rt. The reaction was stirred at rt for 2 h, quenched with a mixture of sodium bisulfate and saturated aqueous sodium thiosulfate, filtered and extracted with EtOAc twice. The combined organic layers were washed with brine, dried, filtered and concentrated to dryness. The residue was used without purification.

Step 3: Methyl (S)-4-(3-hydroxypyrrolidine-1-carbonyl)-6-(trifluoromethyl)picolinate. A mixture of 2-(methoxycarbonyl)-6-(trifluoromethyl)isonicotinic acid (0.794 g, 3.19 mmol), triethylamine (0.889 mL, 6.37 mmol) and EDC (3.70 g, 19.1 mmol) and 4-(dimethylamino)pyridine (0.039 g, 0.319 mmol) in dichloromethane (15.9 mL) was stirred at rt for 14 h. Purification on silica gel (methanol in dichloromethane 0% to 10%) afforded the title compound as a brown oil. Yield: 0.130 g (12.8%).

Step 4: 4-((S)-3-hydroxypyrrolidine-1-carbonyl)-N-(3-((R)-1-(4-methyl-4H-1,2,4-triazol-3-yl)propan-2-yl)phenyl)-6-(trifluoromethyl)picolinamide. Followed Example 103 using D-a and methyl 4-[(3S)-3-hydroxypyrrolidine-1-carbonyl]-6-(trifluoromethyl)pyridine-2-carboxylate (0.130 g, 0.409 mmol) to afforded the title compound. Yield: 0.0718 g (34.9%). ¹H NMR (500 MHz, DMSO-d6) δ 10.41 (s, 1H), 8.38 (dd, J=5.4, 1.3 Hz, 1H), 8.34 (s, 1H), 8.25 (dd, J=3.1, 1.4 Hz, 1H), 7.80-7.71 (m, 2H), 7.31 (t, J=7.8 Hz, 1H), 7.07 (dt, J=7.6, 1.3 Hz, 1H), 5.06 (m, 1H), 4.32 (m, 1H), 3.66-3.50 (m, 2H), 3.46 (s, 3H), 3.45-3.22 (m, 2H), 3.18 (m, 1H), 3.10 (m, 1H), 3.01 (m, 1H), 2.04-1.75 (m, 2H), 1.30 (d, J=6.9 Hz, 3H). LCMS: C₂₄H₂₅F₃N₆O₃ requires: 502, found: m/z=503 [M+H]⁺.

### Example 221: 6-Cyclopropyl-4-[(3S)-3-hydroxypyrrolidine-1-carbonyl]-N-[3-[(1S)-1-methyl-2-(4-methyl-1,2,4-triazol-3-yl)ethyl]phenyl]pyridine-2-carboxamide (221)

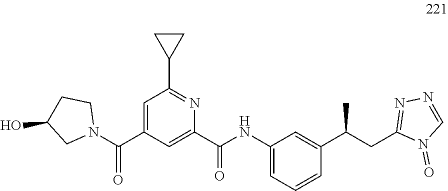

Step 1: Methyl 2-chloro-6-vinyl-pyridine-4-carboxylate. To a stirring solution of vinylboronic anhydride pyridine complex (2.3 g, 9.7 mmol) and methyl 2,6-dichloropyridine-4-carboxylate (4 g, 19.4 mmol) in dimethoxyethane (100 mL) was added tetrakis(triphenylphosphine)palladium(0) (1.12 g, 0.97 mmol) and potassium carbonate (2.24 mL, 38.8 mmol). The reaction was allowed to proceed at 100° C. for about 4 h under nitrogen, cooled to rt, and then concentrated. The reaction was diluted with 1 M hydrochloric acid, followed by General Work-up Procedure 1. The crude was purified using flash column chromatography 20-60% EtOAc/hexanes to the title compound (1.85 g, 48% yield) as a colorless solid.

Step 2: 6-Chloro-4-(methoxycarbonyl)picolinic acid. To a solution of methyl 2-chloro-6-vinyl-pyridine-4-carboxylate (1.85 g, 9.36 mmol, 1 eq.) in water (50 mL) and acetone (50 mL), was added potassium permanganate (7.4 g, 46.8 mmol, 5 eq.). The reaction was allowed to proceed at rt for about 3 h. The reaction was quenched with sodium thiosulfate, the pH was adjusted to 2 using 1 M hydrochloric acid, followed by General Work-up Procedure 1. The crude material was used

Step 3: Dimethyl 6-chloropyridine-2,4-dicarboxylate. To a solution 6-chloro-4-(methoxycarbonyl)picolinic acid in methanol (150 mL) was added sulfuric acid (2 mL). The reaction was allowed to proceed at 70° C. for about 18 h. The reaction was cooled to rt, diluted with water followed by General Work-up Procedure 1. The remaining residue was purified using flash column chromatography eluting from 0-40% EtOAc/hexanes to give the title compound (130 mg, 6% yield)

Step 4: Dimethyl 6-cyclopropylpyridine-2,4-dicarboxylate. To a solution of dimethyl 6-chloropyridine-2,4-dicarboxylate (700 mg, 3.05 mmol), potassium carbonate (1.30 g, 9.15 mmol), cyclopropylboronic acid pinacol ester (1.67 mL, 9.15 mmol) in dioxane (15 mL) and water (1 mL) under nitrogen was added 1,1′-bis(diphenylphosphino)ferrocene-palladium(II)dichloride dichloromethane complex (258 mg, 0.30 mmol). The reaction was allowed to proceed at 100° C. for about 4 h, cooled to rt, and then concentrated. The reaction was diluted with water, followed by General Work-up Procedure 1. The crude was purified using flash column chromatography eluting from 0-50% EtOAc/hexanes to give the title compound (130 mg, 18% yield) as a colorless solid.

Step 5: 6-Cyclopropyl-4-methoxycarbonyl-pyridine-2-carboxylic acid. To a solution of dimethyl 6-cyclopropylpyridine-2,4-dicarboxylate (130 mg, 0.55 mmol) in methanol (3.2 mL) was added sodium hydroxide (15 mg, 0.61 mmol) in water (3.2 mL). The mixture was stirred at rt for 12 h. The reaction was diluted with 1 M hydrochloric acid, followed by General Work-up Procedure 1. The crude was used.

Step 6: Methyl (S)-2-cyclopropyl-6-((3-(1-(4-methyl-4H-1,2,4-triazol-3-yl)propan-2-yl)phenyl)carbamoyl)isonicotinate: followed General procedure 6 using D-b to afford the title compound (122 mg, 0.29 mmol, 53% yield) as a yellow solid.

Step 7: (S)-2-Cyclopropyl-6-((3-(1-(4-methyl-4H-1,2,4-triazol-3-yl)propan-2-yl)phenyl)carbamoyl)isonicotinic acid. To a solution of methyl (S)-2-cyclopropyl-6-((3-(1-(4-methyl-4H-1,2,4-triazol-3-yl)propan-2-yl)phenyl)carbamoyl)isonicotinate (16 mg, 0.04 mmol) in THF (0.4 mL) was added water (0.4 mL) and lithium hydroxide (10 mg, 0.40 mmol). The mixture was stirred at rt for 12 h. The reaction was diluted with 1 M hydrochloric acid, followed by General Work-up Procedure 1. The crude was used without purification.

Step 8: 6-Cyclopropyl-4-[(3S)-3-hydroxypyrrolidine-1-carbonyl]-N-[3-[(1S)-1-methyl-2-(4-methyl-1,2,4-triazol-3-yl)ethyl]phenyl]pyridine-2-carboxamide. Followed General procedure 1-G using (5)-3-hydroxypyrrolidine to afford the title compound (5.8 mg, 31% yield). ¹H NMR (500 MHz, methanol-d4) δ 8.86 (s, 1H), 8.00 (dd, J=7.6, 1.4 Hz, 1H), 7.69 (t, J=1.9 Hz, 1H), 7.54 (td, J=7.1, 1.3 Hz, 2H), 7.33 (t, J=7.9 Hz, 1H), 7.05 (d, J=7.7 Hz, 1H), 4.55-4.35 (m, 1H), 3.85-3.55 (m, 6H), 3.50-3.33 (m, 6H), 2.30 (th, J=6.5, 1.6 Hz, 1H), 2.17-1.97 (m, 2H), 1.48 (d, J=6.7 Hz, 3H), 1.20 (tt, J=5.2, 2.2 Hz, 2H), 1.15 (dt, J=8.2, 2.9 Hz, 2H). LCMS: C₂₆H₃₀N₆O₃ requires: 474, found: m/z=475 [M+H]⁺

### Example 222: 4-((S)-3-hydroxy-3-methylpyrrolidine-1-carbonyl)-N-(3-((R)-1-(4-methyl-4H-1,2,4-triazol-3-yl)propan-2-yl)phenyl)picolinamide (222)

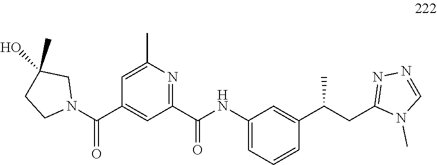 [SMILES: Cc1cc(C(=O)N2CC[C@](C)(O)C2)cc(C(=O)Nc2cccc([C@H](C)Cc3nncn3C)c2)n1]

Followed General procedure 7 to afford the title compound (14.3 mg, 9%) as a colorless solid. MS (ESI) calculated for (C₂₄H₂₈N₆O₃) [M+H]⁺, 449; found, 449. ¹H NMR (300 MHz, DMSO-4, (contain D₂O)) δ 8.85 (d, J=4.8 Hz, 1H), 8.29 (s, 1H), 8.16-8.14 (m, 1H), 7.77-7.73 (m, 3H), 7.34-7.28 (m, 1H), 7.04 (d, J=7.5 Hz, 1H), 3.68-3.11 (m, 5H), 3.41 (s, 3H), 3.00 (d, J=7.5 Hz, 2H), 1.92-1.86 (m, 2H), 1.38-1.20 (m, 6H).

### General Procedure 7:

To a mixture of J-a·HCl (250 mg, 0.68 mmol), Amine (0.82 mmol), HATU (390 mg, 1.03 mmol) and N,N-diisopropylethylamine (265 mg, 2.05 mmol) in N,N-dimethylformamide (5 mL) was stirred at rt for 2-18 h and monitored by LCMS. The mixture was diluted by the addition of water, followed by General Work-up Procedure 1. The residue was purified by flash column chromatography to afford the crude product. The crude product was purified by Prep-HPLC to afford the desired product.

### Example 223: 4-((2S,4S)-4-hydroxy-2-methylpyrrolidine-1-carbonyl)-N-(3-((R)-1-(4-methyl-4H-1,2,4-triazol-3-yl)propan-2-yl)phenyl)picolinamide (223)

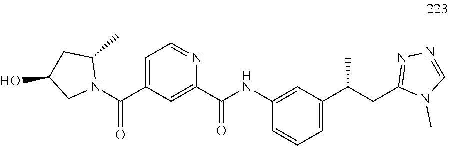 [SMILES: C[C@H](Cc1nncn1C)c1cccc(NC(=O)c2cc(C(=O)N3C[C@@H](O)C[C@@H]3C)ccn2)c1]

Followed General procedure 7 to afford 223 (58 mg, 35%) as a colorless solid. 1H NMR (300 MHz, DMSO-d₆) δ 10.62 (s, 1H), 8.83 (d, J=4.8 Hz, 1H), 8.36 (s, 1H), 8.13 (s, 1H), 7.82-7.74 (m, 3H), 7.30 (t, J=7.8 Hz, 1H), 7.02 (d, J=7.8 Hz, 1H), 5.03 (s, 1H), 4.40-4.03 (m, 2H), 3.73-3.54 (m, 2H), 3.47-3.22 (m, 4H), 3.14-3.09 (m, 1H), 3.01-2.98 (m, 2H), 2.16-2.03 (m, 1H), 1.73-1.68 (m, 1H), 1.33-1.28 (m, 5H). MS (ESI) calculated for (C₂₄H₂₈N₆O₃) [M+H]⁺, 449; found, 449.

### Example 224: 4-((2R,4S)-4-hydroxy-2-methylpyrrolidine-1-carbonyl)-N-(3-((R)-1-(4-methyl-4H-1,2,4-triazol-3-yl)propan-2-yl)phenyl)picolinamide (224)

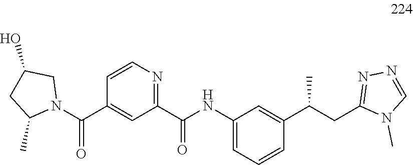 [SMILES: C[C@H](Cc1nncn1C)c1cccc(NC(=O)c2cc(C(=O)N3C[C@@H](O)C[C@H]3C)ccn2)c1]

Step 1: Synthesis of (3S,5R)-5-methylpyrrolidin-3-ol hydrochloride. A mixture of tert-butyl (2R,4S)-4-hydroxy-2-methylpyrrolidine-1-carboxylate (500.0 mg, 2.48 mmol) in hydrochloric acid (10 mL, 4 M in dioxane) was stirred at rt for 2 h. The mixture was evaporated in vacuo to afford the title compound (450.0 mg, crude) as a colorless solid, which was used without purification.

Step 2: Synthesis of 4-((2R,4S)-4-hydroxy-2-methylpyrrolidine-1-carbonyl)-N-(3-((R)-1-(4-methyl-4H-1,2,4-triazol-3-yl)propan-2-yl)phenyl)picolinamide. Followed General procedure 7 to afford 4-((2R,4S)-4-hydroxy-2-methylpyrrolidine-1-carbonyl)-N-(3-((R)-1-(4-methyl-4H-1,2,4-triazol-3-yl)propan-2-yl)phenyl)picolinamide (68 mg, 43%) as a colorless solid. ¹H NMR (400 MHz, DMSO-d₆) δ 10.62 (s, 1H), 8.83 (d, J=4.8 Hz, 1H), 8.29 (s, 1H), 8.13 (s, 1H), 7.83-7.74 (m, 3H), 7.30-7.26 (m, 1H), 7.02 (d, J=7.6 Hz, 1H), 5.14 (s, 1H), 4.46-4.13 (m, 2H), 3.44-3.15 (m, 6H), 2.98 (d, J=7.2 Hz, 2H), 2.31-2.24 (m, 1H), 1.64-1.54 (m, 1H), 1.40 (d, J=6.4 Hz, 2H), 1.29 (d, J=6.8 Hz, 3H), 1.00 (d, J=6.4 Hz, 1H). MS (ESI) calculated for (C₂₄H₂₈N₆O₃) [M+H]⁺, 449; found, 449.

### Example 225: 4-(3-methanesulfonylazetidine-1-carbonyl)-N-[3-[(2R)-1-(4-methyl-4H-1,2,4-triazol-3-yl)propan-2-yl]phenyl]pyridine-2-carboxamide (225)

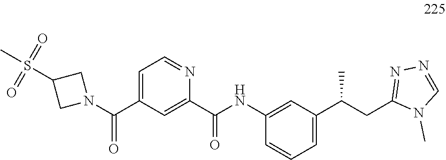 [SMILES: C[C@H](Cc1nncn1C)c1cccc(NC(=O)c2cc(C(=O)N3CC(S(C)(=O)=O)C3)ccn2)c1]

Followed General procedure 7 to afford 225 (47 mg, 27%) as a colorless solid. 1H NMR (300 MHz, DMSO-d₆) δ 10.62 (s, 1H), 8.87 (d, J=4.8 Hz, 1H), 8.28-8.25 (m, 2H), 7.92-7.76 (m, 3H), 7.31-7.25 (m, 1H), 7.02 (d, J=7.8 Hz, 1H), 4.80-4.61 (m, 1H), 4.58-4.48 (m, 1H), 4.47-4.29 (m, 3H), 3.45 (s, 3H), 3.28-3.20 (m, 1H), 3.09 (s, 3H), 2.97 (d, J=7.5 Hz, 2H), 1.29 (d, J=6.9 Hz, 3H). MS (ESI) calculated for (C₂₃H₂₆N₆O₄S) [M+H]⁺, 483; found, 483.

### Example 226: (R)-N⁴-(2-hydroxy-2-methylpropyl)-N²-(3-(1-(4-methyl-4H-1,2,4-triazol-3-yl)propan-2-yl)phenyl)pyridine-2,4-dicarboxamide (226)

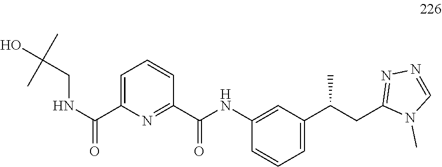 [SMILES: C[C@H](Cc1nncn1C)c1cccc(NC(=O)c2cccc(C(=O)NCC(C)(C)O)n2)c1]

Followed General procedure 7 to afford 226 (71 mg, 47%) as a colorless solid. ¹H NMR (300 MHz, DMSO-d₆) δ 10.63 (s, 1H), 8.88-8.82 (m, 2H), 8.55 (d, J=0.9 Hz, 1H), 8.34 (s, 1H), 8.06-8.03 (m, 1H), 7.83-7.81 (m, 2H), 7.31-7.26 (m, 1H), 7.02 (d, J=7.8 Hz, 1H), 4.58 (s, 1H), 3.47 (s, 3H), 3.35-3.22 (m, 3H), 2.99 (d, J=7.2 Hz, 2H), 1.29 (d, J=6.6 Hz, 3H), 1.13 (s, 6H). MS (ESI) calc'd for (C₂₃H₂₈N₆O₃) [M+H]⁺, 437; found, 437.

### Example 227: (R)-4-(4-hydroxy-4-methylpiperidine-1-carbonyl)-N-(3-(1-(4-methyl-4H-1,2,4-triazol-3-yl)propan-2-yl)phenyl)picolinamide (227)

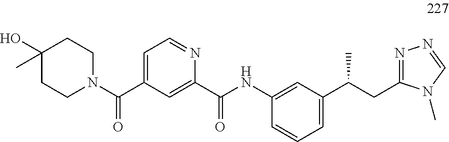 [SMILES: C[C@H](Cc1nncn1C)c1cccc(NC(=O)c2cc(C(=O)N3CCC(C)(O)CC3)ccn2)c1]

Followed General procedure 7 to afford 227 (59 mg, 35%) as a colorless solid. 1H NMR (400 MHz, DMSO-d₆+D₂O) δ 8.80 (d, J=4.2 Hz, 1H), 8.31 (s, 1H), 8.12-8.01 (m, 1H), 7.71-7.69 (m, 2H), 7.63-7.61 (m, 1H), 7.30-7.25 (m, 1H), 7.01 (d, J=7.6 Hz, 1H), 4.10-4.02 (m, 1H), 3.43 (s, 3H), 3.36-3.16 (m, 4H), 2.99 (d, J=7.2 Hz, 2H), 1.62-1.40 (m, 4H), 1.27 (d, J=6.8 Hz, 3H), 1.16 (s, 3H). MS (ESI) calculated for (C₂₅H₃₀N₆O₃) [M+H]⁺, 463; found, 463.

### Example 228: N2-(3-((R)-1-(4-methyl-4H-1,2,4-triazol-3-yl)propan-2-yl)phenyl)-N4-(tetrahydrofuran-3-yl)pyridine-2,4-dicarboxamide (228)

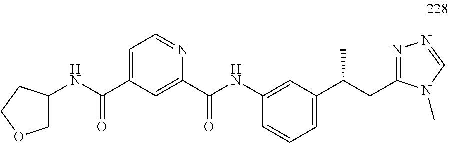 [SMILES: C[C@H](Cc1nncn1C)c1cccc(NC(=O)c2cc(C(=O)NC3CCOC3)ccn2)c1]

Followed General procedure 7 to afford 228 (62 mg, 38%) as a colorless solid. ¹H NMR (300 MHz, DMSO-d₆) δ 10.63 (s, 1H), 9.13 (d, J=6.6 Hz, 1H), 8.88 (d, J=5.1 Hz, 1H), 8.58 (d, J=1.2 Hz, 1H), 8.34 (s, 1H), 8.06-8.03 (m, 1H), 7.83-7.81 (m, 2H), 7.32-7.26 (m, 1H), 7.02 (d, J=7.5 Hz, 1H), 4.59-4.45 (m, 1H), 3.90-3.84 (m, 2H), 3.79-3.63 (m, 2H), 3.46 (s, 3H), 3.26-3.21 (m, 1H), 2.99 (d, J=7.5 Hz, 2H), 2.22-2.15 (m, 1H), 2.08-1.96 (m, 1H), 1.29 (d, J=6.9 Hz, 3H). MS (ESI) calculated for (C₂₃H₂₆N₆O₃) [M+H]⁺, 435; found, 435.

### Example 229: 4-((S)-3-amino-3-methylpyrrolidine-1-carbonyl)-N-(3-((R)-1-(4-methyl-4H-1,2,4-triazol-3-yl)propan-2-yl)phenyl)picolinamide (229)

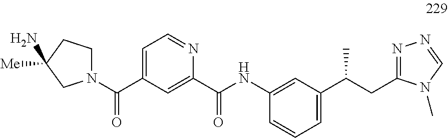 [SMILES: C[C@H](Cc1nncn1C)c1cccc(NC(=O)c2cc(C(=O)N3CC[C@](C)(N)C3)ccn2)c1]

Compound 229 (34.5 mg, 28%) was obtained according to procedures disclosed herein as a colorless solid. ¹H NMR (300 MHz, DMSO-d₆) δ 10.61 (s, 1H), 8.84-8.82 (m, 1H), 8.30 (s, 1H), 8.19-8.14 (m, 1H), 7.82-7.71 (m, 3H), 7.30-7.25 (m, 1H), 7.02 (d, J=7.8 Hz, 1H), 3.76-3.50 (m, 2H), 3.41 (s, 3H), 3.32-3.06 (m, 3H), 2.97 (d, J=7.5 Hz, 2H), 1.95-1.70 (m, 4H), 1.34-1.24 (m, 4H), 1.16 (s, 2H). MS (ESI) calculated for (C₂₄H₂₉N₇O₂) [M+H]⁺, 448; found, 448.

### Example 230: 4-((R)-3-amino-3-methylpyrrolidine-1-carbonyl)-N-(3-((R)-1-(4-methyl-4H-1,2,4-triazol-3-yl)propan-2-yl)phenyl)picolinamide 2,2,2-trifluoroacetate (230)

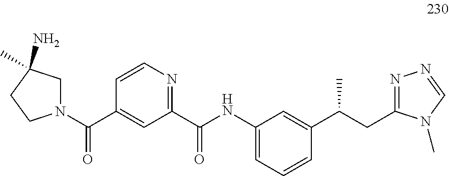 [SMILES: C[C@H](Cc1nncn1C)c1cccc(NC(=O)c2cc(C(=O)N3CC[C@@](C)(N)C3)ccn2)c1]

Compound 230 (17 mg, 26%) was obtained according to procedures disclosed herein as a colorless solid. ¹H NMR (500 MHz, DMSO-d₆) δ 10.63 (s, 1H), 8.92-8.78 (m, 2H), 8.33-8.05 (m, 4H), 7.86-7.71 (m, 3H), 7.30 (t, J=7.8 Hz, 1H), 7.05 (dd, J=7.7, 1.4 Hz, 1H), 3.89-3.62 (m, 2H), 3.59 (s, 3H), 3.57-3.43 (m, 2H), 3.28 (d, J=7.0 Hz, 1H), 3.20-3.08 (m, 2H), 2.28-2.01 (m, 2H), 1.41 (d, J=59.2 Hz, 3H), 1.32 (d, J=6.8 Hz, 3H). LCMS: C₂₄H₂₉N₇O₂ requires: 447, found: m/z=448 [M+H]⁺.

### Example 231: 4-(3-hydroxy-2,2-dimethylpyrrolidine-1-carbonyl)-N-(3-((R)-1-(4-methyl-4H-1,2,4-triazol-3-yl)propan-2-yl)phenyl)picolinamide (231)

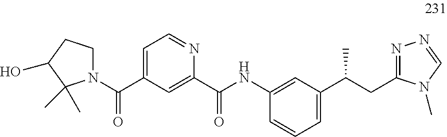 [SMILES: C[C@H](Cc1nncn1C)c1cccc(NC(=O)c2cc(C(=O)N3CCC(O)C3(C)C)ccn2)c1]

Followed General procedure 7 to 231 (65 mg, 10%) as a colorless solid. 1H NMR (300 MHz, DMSO-d₆) δ 10.59 (s, 1H), 8.79 (d, J=4.8 Hz, 1H), 8.27 (s, 1H), 8.06 (s, 1H), 7.81-7.79 (m, 2H), 7.68-7.65 (m, 1H), 7.30-7.25 (m, 1H), 7.01 (d, J=7.8 Hz, 1H), 5.19 (s, 1H), 3.83-3.81 (m, 1H), 3.44 (s, 3H), 3.40-3.32 (m, 1H), 3.27-3.21 (m, 2H), 2.97 (d, J=7.2 Hz, 2H), 2.07-1.93 (m, 1H), 1.70-1.63 (m, 1H), 1.47 (s, 3H), 1.40 (s, 3H), 1.28 (d, J=6.9 Hz, 3H). MS (ESI) calculated for (C₂₅H₃₀N₆O₃) [M+H]⁺, 463; found, 463.

### Example 232: 4-[3-(hydroxymethyl)azetidine-1-carbonyl]-N-[3-[(2R)-1-(4-methyl-4H-1,2,4-triazol-3-yl)propan-2-yl]phenyl]pyridine-2-carboxamide (232)

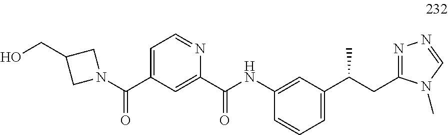 [SMILES: C[C@H](Cc1nncn1C)c1cccc(NC(=O)c2cc(C(=O)N3CC(CO)C3)ccn2)c1]

Followed General procedure 7 to afford 232 (36 mg, 22%) as a colorless solid. 1H NMR (400 MHz, DMSO-d₆) δ 10.63 (s, 1H), 8.85 (d, J=4.8 Hz, 1H), 8.32 (s, 1H), 8.25 (s, 1H), 7.85-7.76 (m, 3H), 7.30-7.26 (m, 1H), 7.02 (t, J=7.6 Hz, 1H), 4.87 (s, 1H), 4.38 (t, J=8.4 Hz, 1H), 4.13-4.06 (m, 2H), 3.87-3.83 (m, 1H), 3.57 (d, J=6.0 Hz, 2H), 3.46 (s, 3H), 3.29-3.20 (m, 1H), 2.99 (d, J=7.6 Hz, 2H), 2.78-2.75 (m, 1H), 1.29 (d, J=6.8 Hz, 3H). MS (ESI) calculated for (C₂₃H₂₆N₆O₃) [M+H]⁺, 435; found, 435.

### Example 233: N-[3-[(2R)-1-(4-methyl-4H-1,2,4-triazol-3-yl)propan-2-yl]phenyl]-4-[2-oxa-6-azaspiro[3.4]octane-6-carbonyl]pyridine-2-carboxamide (233)

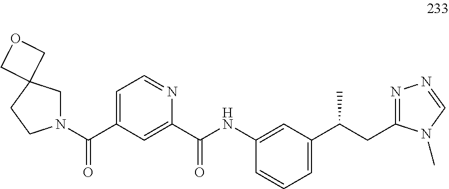 [SMILES: C[C@H](Cc1nncn1C)c1cccc(NC(=O)c2cc(C(=O)N3CCC4(COC4)C3)ccn2)c1]

Followed General procedure 7 to afford 233 (21 mg, 44%) as a colorless solid. 1H NMR (300 MHz, DMSO-d₆) δ 10.61 (d, J=4.8 Hz, 1H), 8.85-8.81 (m, 1H), 8.28 (s, 1H), 8.17 (s, 1H), 7.89-7.70 (m, 3H), 7.27 (t, J=7.8 Hz, 1H), 7.01 (d, J=7.2 Hz, 1H), 4.64 (d, J=6.0 Hz, 1H), 4.51-4.43 (m, 3H), 3.75 (s, 1H), 3.64 (s, 1H), 3.53 (t, J=7.2 Hz, 1H), 3.44 (s, 3H), 3.41-3.31 (m, 1H), 3.31-3.19 (m, 1H), 3.08-2.88 (m, 2H), 2.25-2.15 (m, 2H), 1.28 (d, J=6.3 Hz, 3H). MS (ESI) calculated for (C₂₅H₂₈N₆O₃) [M+H]⁺, 461; found, 461.

### Example 234: 4-[(1R,5S,6R)-6-hydroxy-3-azabicyclo[3.1.0]hexane-3-carbonyl]-N-[3-[(2R)-1-(4-methyl-4H-1,2,4-triazol-3-yl)propan-2-yl]phenyl]pyridine-2-carboxamide (234)

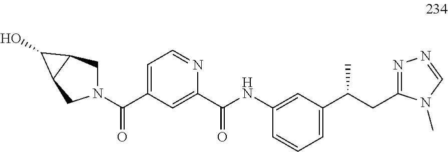 [SMILES: C[C@H](Cc1nncn1C)c1cccc(NC(=O)c2cc(C(=O)N3C[C@@H]4[C@@H](O)[C@@H]4C3)ccn2)c1]

Followed General procedure 7 to afford 234 (18 mg, 11%) as a colorless solid. 1H NMR (300 MHz, DMSO-d₆) δ 10.59 (s, 1H), 8.81-8.80 (m, 1H), 8.28 (s, 1H), 8.10 (s, 1H), 7.81-7.75 (m, 2H), 7.72-7.70 (m, 1H), 7.30-7.25 (m, 1H), 7.01 (d, J=7.8 Hz, 1H), 5.45 (d, J=1.2 Hz, 1H), 3.84-3.80 (m, 1H), 3.64-3.59 (m, 1H), 3.53-3.51 (m, 1H), 3.45 (s, 3H), 3.32-3.19 (m, 2H), 3.03-2.94 (m, 3H), 1.66-1.61 (m, 2H), 1.29 (d, J=6.9 Hz, 3H). MS (ESI) calculated for (C₂₄H₂₆N₆O₃) [M+H]⁺, 447; found, 447.

### Example 235: 4-[(3S,4S)-3-fluoro-4-hydroxypyrrolidine-1-carbonyl]-N-[3-[(2R)-1-(4-methyl-4H-1,2,4-triazol-3-yl)propan-2-yl]phenyl]pyridine-2-carboxamide (235)

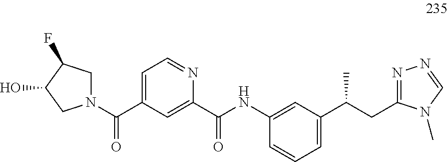 [SMILES: C[C@H](Cc1nncn1C)c1cccc(NC(=O)c2cc(C(=O)N3C[C@H](O)[C@@H](F)C3)ccn2)c1]

Followed General procedure 7 to afford 235 (18 mg, 12%) as a colorless solid. ¹H NMR (300 MHz, DMSO-d₆) δ 10.65 (s, 1H), 8.97 (s, 1H), 8.85 (d, J=5.1 Hz, 1H), 8.16-8.14 (m, 1H), 7.84-7.77 (m, 3H), 7.33-7.27 (m, 1H), 7.05 (d, J=7.5 Hz, 1H), 6.00-5.40 (br, 1H), 5.20-4.90 (m, 1H), 4.25-4.20 (m, 1H), 4.00-3.50 (m, 6H), 3.31-3.21 (m, 1H), 3.21-3.11 (m, 2H), 1.33 (d, J=6.6 Hz, 3H). MS (ESI) calculated for (C₂₃H₂₅FN₆O₃) [M+H]⁺, 453; found, 453.

### Example 236: 4-[(3S)-3-cyanopyrrolidine-1-carbonyl]-N-[3-[(2R)-1-(4-methyl-4H-1,2,4-triazol-3-yl)propan-2-yl]phenyl]pyridine-2-carboxamide (236)

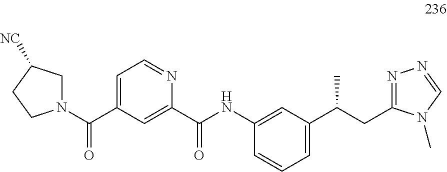 [SMILES: C[C@H](Cc1nncn1C)c1cccc(NC(=O)c2cc(C(=O)N3CC[C@H](C#N)C3)ccn2)c1]

Followed General procedure 7 to afford 236 (53 mg, 32%) as a colorless solid. 1H NMR (300 MHz, DMSO-d₆) δ 10.62 (s, 1H), 8.86-8.84 (m, 1H), 8.46 (s, 1H), 8.20 (s, 1H), 7.82-7.75 (m, 3H), 7.32-7.26 (m, 1H), 7.04-7.01 (m, 1H), 3.92-3.43 (m, 8H), 3.30-3.23 (m, 1H), 3.03 (d, J=7.2 Hz, 2H), 2.36-2.07 (m, 2H), 1.29 (d, J=6.9 Hz, 3H). MS (ESI) calculated for (C₂₄H₂₅N₇O₂) [M+H]⁺, 444; found, 444.

### Example 237: N-[3-[(2R)-1-(4-methyl-4H-1,2,4-triazol-3-yl)propan-2-yl]phenyl]-4-[1-oxa-6-azaspiro[3.4]octane-6-carbonyl]pyridine-2-carboxamide (237)

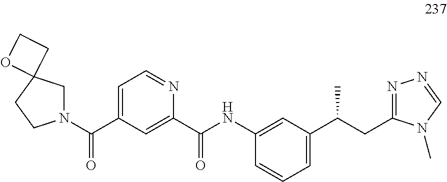 [SMILES: C[C@H](Cc1nncn1C)c1cccc(NC(=O)c2cc(C(=O)N3CCC4(CCO4)C3)ccn2)c1]

Followed General procedure 7 to afford 237 (2 mg, 1%) as a colorless solid. 1H NMR (300 MHz, DMSO-d₆) δ 10.61 (d, J=4.8 Hz, 1H), 8.86-8.81 (m, 1H), 8.28 (s, 1H), 8.14 (d, J=6.0 Hz, 1H), 7.82-7.77 (m, 3H), 7.28-7.25 (m, 1H), 7.02 (d, J=7.8 Hz, 1H), 4.43-4.40 (m, 2H), 3.65-3.60 (m, 2H), 3.47-3.45 (m, 5H), 3.30-3.20 (m, 1H), 2.97 (d, J=7.2 Hz, 2H), 2.98-2.60 (m, 2H), 2.32-2.20 (m, 1H), 2.15-2.00 (m, 1H), 1.29 (d, J=6.9 Hz, 3H). MS (ESI) calculated for (C₂₅H₂₈N₆O₃) [M+H]⁺, 461; found, 461.

### Example 238: 4-((R)-3-cyanopyrrolidine-1-carbonyl)-N-(3-((R)-1-(4-methyl-4H-1,2,4-triazol-3-yl)propan-2-yl)phenyl)picolinamide (238)

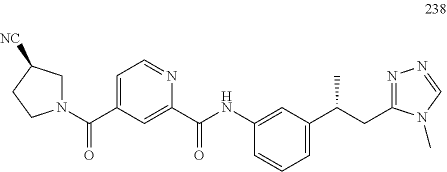 [SMILES: C[C@H](Cc1nncn1C)c1cccc(NC(=O)c2cc(C(=O)N3CC[C@@H](C#N)C3)ccn2)c1]

Followed General procedure 7 to afford 238 (7 mg, 6%) as a colorless solid. 1H NMR (300 MHz, DMSO-d₆) δ 10.63 (s, 1H), 8.87-8.85 (m, 1H), 8.29 (s, 1H), 8.20 (d, J=0.6 Hz, 1H), 7.83-7.78 (m, 3H), 7.32-7.26 (m, 1H), 7.03 (d, J=7.8 Hz, 1H), 3.89-3.58 (m, 5H), 3.49 (s, 3H), 3.46-3.23 (m, 1H), 2.98 (d, J=7.5 Hz, 2H), 2.35-2.18 (m, 2H), 1.30 (d, J=6.9 Hz, 3H). MS (ESI) calculated for (C₂₄H₂₅N₇O₂) [M+H]⁺, 444; found, 444.

### Example 239: 4-(4-hydroxy-3,3-dimethylpyrrolidine-1-carbonyl)-N-(3-((R)-1-(4-methyl-4H-1,2,4-triazol-3-yl)propan-2-yl)phenyl)picolinamide (239)

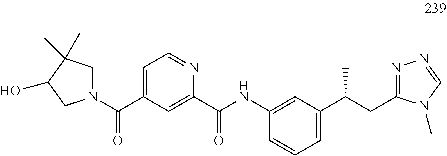 [SMILES: C[C@H](Cc1nncn1C)c1cccc(NC(=O)c2cc(C(=O)N3CC(O)C(C)(C)C3)ccn2)c1]

Followed General procedure 7 to afford 239 (65 mg, 11%) as a colorless solid. 1H NMR (300 MHz, Chloroform-d) δ 9.95 (s, 1H), 8.72-8.70 (m, 1H), 8.34-8.31 (m, 1H), 7.93 (s, 1H), 7.63-7.58 (m, 3H), 7.31-7.26 (m, 1H), 6.95-6.92 (m, 1H), 4.04-3.53 (m, 2H), 3.49-3.42 (m, 2H), 3.42-3.34 (m, 1H), 3.23 (s, 3H), 3.15-3.07 (m, 2H), 2.98-2.93 (m, 1H), 1.47 (d, J=6.9 Hz, 3H), 1.17-0.98 (m, 6H). MS (ESI) calculated for (C₂₅H₃₀N₆O₃) [M+H]⁺, 463; found, 463.

### Example 240: 4-((3R,4R)-3-fluoro-4-hydroxypyrrolidine-1-carbonyl)-N-(3-((K)-1-(4-methyl-4H-1,2,4-triazol-3-yl)propan-2-yl)phenyl)picolinamide (240)

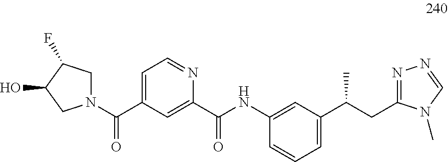 [SMILES: C[C@H](Cc1nncn1C)c1cccc(NC(=O)c2cc(C(=O)N3C[C@@H](O)[C@H](F)C3)ccn2)c1]

Followed General procedure 7 to afford 240 (24 mg, 14%) as a colorless solid. 1H NMR (300 MHz, DMSO-d₆) δ 10.62 (s, 1H), 8.85 (d, J=4.8 Hz, 1H), 8.28 (s, 1H), 8.16 (d, J=0.9 Hz, 1H), 7.82-7.79 (m, 3H), 7.31-7.25 (m, 1H), 7.02 (d, J=7.5 Hz, 1H), 5.70-5.61 (m, 1H), 5.15-4.89 (m, 1H), 4.32-4.24 (m, 1H), 3.98-3.92 (m, 1H), 3.83-3.70 (m, 2H), 3.65-3.52 (m, 1H), 3.45 (s, 3H), 3.29-3.21 (m, 1H), 2.97 (d, J=7.5 Hz, 2H), 1.32 (d, J=6.6 Hz, 3H). MS (ESI) calculated for (C₂₃H₂₅FN₆O₃) [M+H]⁺, 453; found, 453.

### Example 241: 4-(3-(1-hydroxyethyl)azetidine-1-carbonyl)-N-(3-((R)-1-(4-methyl-4H-1,2,4-triazol-3-yl)propan-2-yl)phenyl)picolinamide (241)

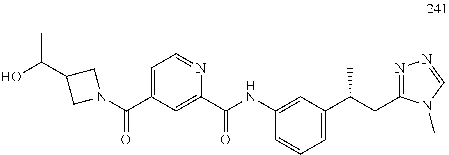 [SMILES: CC(O)C1CN(C(=O)c2ccnc(C(=O)Nc3cccc([C@H](C)Cc4nncn4C)c3)c2)C1]

Compound 241 (3 mg, 1.2%) was obtained according to procedures disclosed herein as a colorless solid. ¹H NMR (300 MHz, DMSO-d₆) δ 10.61 (s, 1H), 8.86-8.84 (m, 1H), 8.33 (s, 1H), 8.24 (s, 1H), 7.90-7.75 (m, 3H), 7.30-7.25 (m, 1H), 7.02 (d, J=7.8 Hz, 1H), 4.80 (br, 1H), 4.35-4.29 (m, 1H), 4.18-3.73 (m, 4H), 3.46 (s, 3H), 3.31-3.20 (m, 1H), 2.99 (d, J=7.2 Hz, 2H), 2.66-2.54 (m, 1H), 1.30 (d, J=6.9 Hz, 3H), 1.02-0.99 (m, 3H). MS (ESI) calculated for (C₂₄H₂₈N₆O₃) [M+H]⁺, 449; found, 449.

### Example 242: (R)-N-(3-(1-(4-methyl-4H-1,2,4-triazol-3-yl)propan-2-yl)phenyl)-4-(piperazine-1-carbonyl)picolinamide (242)

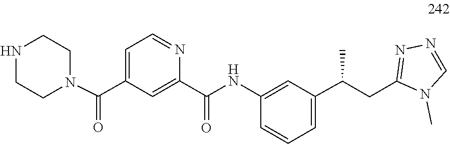 [SMILES: C[C@H](Cc1nncn1C)c1cccc(NC(=O)c2cc(C(=O)N3CCNCC3)ccn2)c1]

Compound 242 (7 mg, 6%) was obtained according to procedures disclosed herein. ¹H NMR (300 MHz, DMSO-d₆) δ 10.61 (s, 1H), 8.81 (d, J=4.8 Hz, 1H), 8.28 (s, 1H), 8.04 (s, 1H), 7.81-7.78 (m, 2H), 7.68-7.66 (m, 1H), 7.30-7.25 (m, 1H), 7.01 (d, J=7.5 Hz, 1H), 3.60-3.45 (m, 2H), 3.33 (s, 3H), 3.25-3.21 (m, 3H), 2.97 (d, J=7.2 Hz, 2H), 2.80-2.70 (m, 2H), 2.70-2.60 (m, 2H), 1.28 (d, J=6.9 Hz, 3H). MS (ESI) calculated for (C₂₃H₂₇N₇O₂) [M+H]⁺, 434; found, 434.

### Example 243: 4-(3,3-difluoro-4-hydroxypyrrolidine-1-carbonyl)-N-[3-[(2R)-1-(4-methyl-4H-1,2,4-triazol-3-yl)propan-2-yl]phenyl]pyridine-2-carboxamide (243)

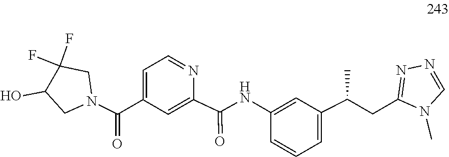 [SMILES: C[C@H](Cc1nncn1C)c1cccc(NC(=O)c2cc(C(=O)N3CC(O)C(F)(F)C3)ccn2)c1]

Step 1: Synthesis of 4,4-difluoropyrrolidin-3-ol hydrochloride. A mixture of tert-butyl 3,3-difluoro-4-hydroxypyrrolidine-1-carboxylate (250 mg, 1.12 mmol) in hydrochloric acid (5 mL, 4 M) was stirred at rt for 2 h. The mixture was evaporated in vacuo to afford the title compound (200 mg) as a yellow solid, which was used.

Step 2: Synthesis of 4-(3,3-difluoro-4-hydroxypyrrolidine-1-carbonyl)-N-[3-[(2R)-1-(4-methyl-4H-1,2,4-triazol-3-yl)propan-2-yl]phenyl]pyridine-2-carboxamide. Followed General procedure 7 to afford the title compound (107 mg, 31%). ¹H NMR (300 MHz, DMSO-d₆) δ 8.88-8.86 (m, 1H), 8.29 (s, 1H), 8.18-8.16 (m, 1H), 7.80-7.75 (m, 3H), 7.34-7.28 (m, 1H), 7.04 (d, J=7.5 Hz, 1H), 4.32-4.25 (m, 1H), 4.00-3.76 (m, 3H), 3.57-3.38 (m, 4H), 3.30-3.23 (m, 1H), 2.90 (d, J=7.2 Hz, 2H), 1.29 (d, J=6.9 Hz, 3H). MS (ESI) calculated for (C_{23C}H₂₄F₂N₆O₃) [M+H]⁺, 471; found, 471.

### Example 244: (R)-N2-(3-(1-(4-methyl-4H-1,2,4-triazol-3-yl)propan-2-yl)phenyl)-N4-(2-(methylamino)ethyl)pyridine-2,4-dicarboxamide (244)

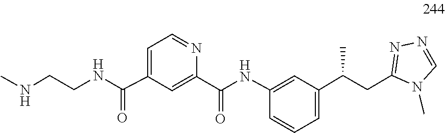 [SMILES: CNCCNC(=O)c1ccnc(C(=O)Nc2cccc([C@H](C)Cc3nncn3C)c2)c1]

Compound 244 (31 mg, 23%) was obtained according to procedures disclosed herein. ¹H NMR (300 MHz, DMSO-d₆) δ 10.61 (s, 1H), 8.99 (s, 1H), 8.85 (d, J=5.1 Hz, 1H), 8.55-8.53 (m, 1H), 8.28 (s, 1H), 8.04-8.02 (m, 1H), 7.83-7.80 (m, 2H), 7.31-7.26 (m, 1H), 7.02 (d, J=7.8 Hz, 1H), 3.50-3.45 (m, 4H), 3.26-3.20 (m, 1H), 2.98 (d, J=6.9 Hz, 2H), 2.78-2.60 (m, 2H), 2.32 (s, 2H), 1.29 (d, J=6.9 Hz, 3H). MS (ESI) calculated for (C₂₂H₂₇N₇O₂) [M+H]⁺, 422; found, 422.

### Example 245: 4-(3-(hydroxymethyl)pyrrolidine-1-carbonyl)-N-(3-((R)-1-(4-methyl-4H-1,2,4-triazol-3-yl)propan-2-yl)phenyl)picolinamide (245)

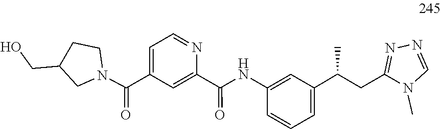 [SMILES: C[C@H](Cc1nncn1C)c1cccc(NC(=O)c2cc(C(=O)N3CCC(CO)C3)ccn2)c1]

Compound 245 (71 mg, 44%) was obtained according to procedures disclosed herein as a colorless solid. 1H NMR (300 MHz, DMSO-D₆+D₂O) δ 8.84 (s, 1H), 8.29 (s, 1H), 8.16 (s, 1H), 7.85-7.70 (m, 3H), 7.32-7.29 (m, 1H), 7.03 (d, J=6.9 Hz, 1H), 3.61-3.45 (m, 7H), 3.40-3.20 (m, 3H), 2.99 (d, J=7.2 Hz, 2H), 2.41-2.32 (m, 1H), 2.05-1.90 (m, 1H), 1.72-1.65 (m, 1H), 1.29 (d, J=6.9 Hz, 3H). MS (ESI) calculated for (C₂₄H₂₈N₆O₃) [M+H]⁺, 449; found, 449.

### Example 246: cis-4-(3-fluoro-4-hydroxypyrrolidine-1-carbonyl)-N-(3-((R)-1-(4-methyl-4H-1,2,4-triazol-3-yl)propan-2-yl)phenyl)picolinamide (246)

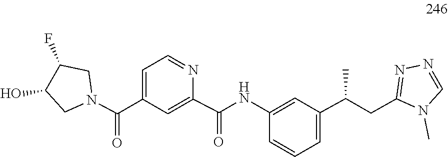 [SMILES: C[C@H](Cc1nncn1C)c1cccc(NC(=O)c2cc(C(=O)N3C[C@@H](F)[C@@H](O)C3)ccn2)c1]

Compound 246 (23 mg, 14%) was obtained according to procedures disclosed herein as a colorless solid. ¹H NMR (300 MHz, DMSO-d₆) δ 10.65 (s, 1H), 8.85 (d, J=4.8 Hz, 1H), 8.28 (s, 1H), 8.18-8.16 (m, 1H), 7.82-7.77 (m, 3H), 7.31-7.26 (m, 1H), 7.04-7.01 (m, 1H), 5.59-5.56 (m, 1H), 5.18-4.82 (m, 1H), 4.48-4.12 (m, 1H), 3.87-3.70 (m, 2H), 3.61-3.51 (m, 1H), 3.45 (s, 3H), 3.39-3.35 (m, 1H), 3.29-3.22 (m, 1H), 2.98 (d, J=7.2 Hz, 2H), 1.29 (d, J=6.9 Hz, 3H). MS (ESI) calculated for (C₂₃H₂₅FN₆O₃) [M+H]⁺, 453; found, 453.

### Example 247: N2-[3-[(2R)-1-(4-methyl-4H-1,2,4-triazol-3-yl)propan-2-yl]phenyl]-N4-[(3R)-pyrrolidin-3-yl]pyridine-2,4-dicarboxamide (247)

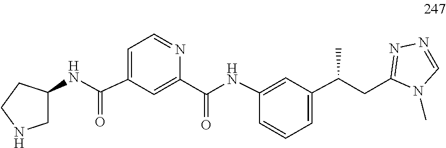 [SMILES: C[C@H](Cc1nncn1C)c1cccc(NC(=O)c2cc(C(=O)N[C@@H]3CCNC3)ccn2)c1]

Compound 247 (59 mg, 36%) was obtained according to procedures disclosed herein. ¹H NMR (300 MHz, DMSO-d₆+D₂O) δ 8.88 (d, J=5.1 Hz, 1H), 8.46 (s, 1H), 8.29 (s, 1H), 7.98-7.96 (m, 1H), 7.71-7.69 (m, 2H), 7.37-7.31 (m, 1H), 7.08-7.05 (m, 1H), 4.23-4.19 (m, 1H), 3.45 (s, 3H), 3.35-3.20 (m, 1H), 3.12-2.92 (m, 4H), 2.91-2.70 (m, 2H), 2.22-2.10 (m, 1H), 1.90-1.83 (m, 1H) 1.31 (d, J=6.9 Hz, 3H). MS (ESI) calculated for (C₂₃H₂₇N₇O₂) [M+H]⁺, 434; found, 434.

### Example 248: N2-(3-((R)-1-(4-methyl-4H-1,2,4-triazol-3-yl)propan-2-yl)phenyl)-N4-((s)-pyrrolidin-3-yl)pyridine-2,4-dicarboxamide (248)

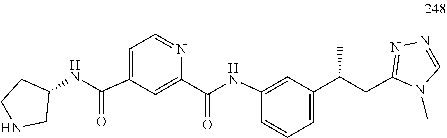 [SMILES: C[C@H](Cc1nncn1C)c1cccc(NC(=O)c2cc(C(=O)N[C@H]3CCNC3)ccn2)c1]

Compound 248 (35 mg, 21%) was obtained according to procedures disclosed herein. ¹H NMR (300 MHz, DMSO-d₆) δ 10.61 (s, 1H), 9.11-8.99 (m, 1H), 8.87 (d, J=4.8 Hz, 1H), 8.57 (s, 1H), 8.28 (s, 1H), 8.04 (d, J=4.5 Hz, 1H), 7.83-7.81 (m, 2H), 7.31-7.25 (m, 1H), 7.02 (d, J=7.5 Hz, 1H), 4.47-4.37 (m, 1H), 3.81-3.68 (m, 4H), 3.28-3.18 (m, 2H), 3.08-2.73 (m, 5H), 2.12-1.71 (m, 2H), 1.29 (d, J=6.9 Hz, 3H). MS (ESI) calculated for (C₂₃H₂₇N₇O₂) [M+H]⁺, 434; found, 434.

### Example 249: N-(3-((R)-1-(4-methyl-4H-1,2,4-triazol-3-yl)propan-2-yl)phenyl)-4-((R)-3-(methylamino)pyrrolidine-1-carbonyl)picolinamide (249)

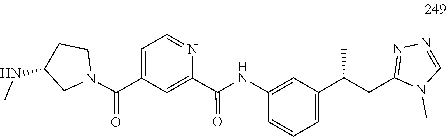 [SMILES: CN[C@@H]1CCN(C(=O)c2ccnc(C(=O)Nc3cccc([C@H](C)Cc4nncn4C)c3)c2)C1]

Compound 249 (40 mg, 25%) was obtained according to procedures disclosed herein as a colorless solid. ¹H NMR (300 MHz, DMSO-d₆) δ 10.60 (s, 1H), 8.84-8.81 (m, 1H), 8.28 (s, 1H), 8.15-8.14 (m, 1H), 7.81-7.75 (m, 3H), 7.30-7.25 (m, 1H), 7.02 (d, J=7.8 Hz, 1H), 3.68-3.45 (m, 6H), 3.36-3.33 (m, 1H), 3.23-3.12 (m, 3H), 2.97 (d, J=7.5 Hz, 2H), 2.29-2.18 (m, 3H), 2.02-1.91 (m, 1H), 1.80-1.64 (m, 1H), 1.29 (d, J=6.9 Hz, 3H). MS (ESI) calculated for (C₂₄H₂₉N₇O₂) [M+H]⁺, 448; found, 448.

### Example 250: N-[3-[(2R)-1-(4-methyl-4H-1,2,4-triazol-3-yl)propan-2-yl]phenyl]-4-[(3S)-3-(methylamino)pyrrolidine-1-carbonyl]pyridine-2-carboxamide (250)

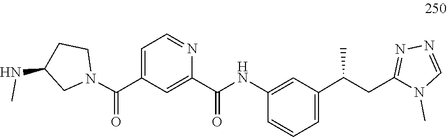 [SMILES: CN[C@H]1CCN(C(=O)c2ccnc(C(=O)Nc3cccc([C@H](C)Cc4nncn4C)c3)c2)C1]

Compound 250 (43 mg, 31%) was obtained according to procedures disclosed herein. ¹H NMR (300 MHz, DMSO-d₆) δ 10.60 (s, 1H), 8.84-8.83 (m, 1H), 8.27 (s, 1H), 8.16-8.14 (m, 1H), 7.81-7.73 (m, 3H), 7.30-7.25 (m, 1H), 7.03-7.00 (m, 1H), 3.63-3.47 (m, 6H), 3.44-3.40 (m, 1H), 3.30-3.19 (m, 2H), 3.18-3.11 (m, 1H), 2.97 (d, J=7.5 Hz, 2H), 2.30-2.19 (m, 3H), 2.00-1.90 (m, 1H), 1.78-1.70 (m, 1H), 1.28 (d, J=6.9 Hz, 3H). MS (ESI) calculated for (C₂₄H₂₉N₇O₂) [M+H]⁺, 448; found, 448.

### Example 251: N-(3-((R)-1-(4-ethyl-4H-1,2,4-triazol-3-yl)propan-2-yl)phenyl)-4-((2R,4S)-4-hydroxy-2-methylpyrrolidine-1-carbonyl)picolinamide (251)

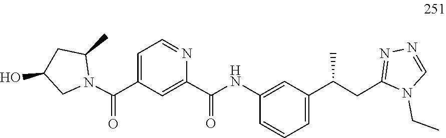 [SMILES: CCn1cnnc1C[C@@H](C)c1cccc(NC(=O)c2cc(C(=O)N3C[C@@H](O)C[C@H]3C)ccn2)c1]

Step 1: (R)-3-(3-nitrophenyl)butanehydrazide. To a solution of (R)-3-((R)-3-(3-nitrophenyl)butanoyl)-4-phenyloxazolidin-2-one (intermediate D-a) (20.0 g, 56.51 mmol) in THF (200 mL) was added hydrazine hydrate (10.6 g, 169.53 mmol, 80%) at rt. After stirred for 16 h, the mixture was concentrated to afford the title compound (22.0 g) as yellow solid, which was used without purification.

Step 2: (R)-4-ethyl-5-(2-(3-nitrophenyl)propyl)-4H-1,2,4-triazole-3-thiol. To a solution of (R)-3-(3-nitrophenyl)butanehydrazide (22.0 g, 98.71 mmol) in anhydrous THF (200 mL) was added isothiocyanatoethane (8.6 g, 98.71 mmol) at rt. After stirred for 16 h, the mixture was concentrated. Sodium hydroxide (200 mL, 1 M) was added to the above residue and stirred for another 2 h. The mixture was acidified by hydrochloric acid (aqueous, 3 A) to pH 56. General Work-up Procedure 1 was followed to afford the title compound (24 g) as a yellow solid, which was used without purification

Step 3: (R)-4-ethyl-3-(2-(3-nitrophenyl)propyl)-4H-1,2,4-triazole. To a solution of (R)-4-ethyl-5-(2-(3-nitrophenyl)propyl)-4H-1,2,4-triazole-3-thiol (24.0 g, 82.2 mmol) in dichloromethane (240 mL) and acetic acid (120 mL) was added hydrogen peroxide (28.0 g, 24.6 mmol, 30%) dropwise at 0° C. After stirred at rt for 1 h, the mixture was concentrated. The residue was purified by flash column chromatography with 0-50% EtOAc in petroleum ether to afford the title compound (14 g, 65%) as a yellow oil.

Step 4: (R)-3-(1-(4-ethyl-4H-1,2,4-triazol-3-yl)propan-2-yl)aniline. A mixture of (R)-4-ethyl-3-(2-(3-nitrophenyl)propyl)-4H-1,2,4-triazole (14 g, 53.7 mmol) and palladium on carbon (4 g, wet, 10%) in methanol (140 mL) was stirred at rt for 16 h under a hydrogen atmosphere. The solids were filtered off. The filtrate was concentrated to afford the title compound (11.5 g, 93%) as a light yellow oil.

Step 5: tert-butyl (R)-2-((3-(1-(4-ethyl-4H-1,2,4-triazol-3-yl)propan-2-yl)phenyl)carbamoyl)isonicotinate. Followed General procedure 1-G using (R)-3-(1-(4-ethyl-4H-1,2,4-triazol-3-yl)propan-2-yl)aniline and 4-(tert-butoxycarbonyl)picolinic acid to afford the title compound (300 mg, 79%).

Step 6: (R)-2-((3-(1-(4-ethyl-4H-1,2,4-triazol-3-yl)propan-2-yl)phenyl)carbamoyl)isonicotinic acid. A solution of tert-butyl (R)-2-((3-(1-(4-ethyl-4H-1,2,4-triazol-3-yl)propan-2-yl)phenyl)carbamoyl)isonicotinate (300 mg, 0.22 mmol) in hydrochloric acid (5 mL, 4 M in dioxane) was stirred at rt for 16 h. The mixture was concentrated to afford the title compound as a yellow oil, which was used without purification.

Step 7: N-(3-((K)-1-(4-ethyl-4H-1,2,4-triazol-3-yl)propan-2-yl)phenyl)-4-((2R,4S)-4-hydroxy-2-methylpyrrolidine-1-carbonyl)picolinamide. Followed General procedure 1-G using (R)-2-((3-(1-(4-ethyl-4H-1,2,4-triazol-3-yl)propan-2-yl)phenyl)carbamoyl)isonicotinic acid and (3S,5R)-5-methylpyrrolidin-3-ol to afford the title compound (22 mg, 18%). ¹H NMR (300 MHz, DMSO-d₆) δ 10.60 (s, 1H), 8.83 (d, J=4.8 Hz, 1H), 8.38 (s, 1H), 8.13 (s, 1H), 7.89-7.67 (m, 3H), 7.28 (t, J=7.8 Hz, 1H), 7.04 (d, J=7.8 Hz, 1H), 5.11 (d, J=3.6 Hz, 1H), 4.44-3.78 (m, 4H), 3.48-3.43 (m, 1H), 3.26-3.24 (m, 2H), 2.98 (d, J=7.2 Hz, 2H), 2.29-2.25 (m, 1H), 1.69-1.50 (m, 1H), 1.41 (d, J=6.3 Hz, 2H), 1.37-1.10 (m, 6H), 1.01 (d, J=6.6 Hz, 1H). MS (ESI) calc'd for (C₂₅H₃₀N₆O₃) [M+H]⁺, 463; found, 463.

### Example 252: 4-((S)-3-hydroxypyrrolidine-1-carbonyl)-N-(3-(1-(1-methyl-s1H-imidazol-2-yl)propan-2-yl)phenyl)picolinamide (252)

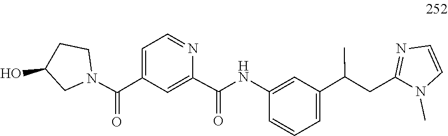 [SMILES: CC(Cc1nccn1C)c1cccc(NC(=O)c2cc(C(=O)N3CC[C@H](O)C3)ccn2)c1]

Followed General procedure 1-G using (5)-pyrrolidin-3-ol to afford the title compound (17 mg, 14%). ¹H NMR (300 MHz, DMSO-d₆+D₂O) δ (8.82 (d, J=4.8 Hz, 1H), 8.13 (s, 1H), 7.73-7.71 (m, 3H), 7.29-7.24 (m, 1H), 6.99-6.97 (m, 1H), 6.92 (d, J=1.2 Hz, 1H), 6.74 (d, J=1.2 Hz, 1H), 4.35-4.28 (m, 1H), 3.72-3.50 (m, 3H), 3.46-3.38 (m, 1H), 3.34 (s, 3H), 3.21-3.12 (m, 1H), 2.88-8.85 (m, 2H), 1.92-1.83 (m, 2H), 1.22 (d, J=6.9 Hz, 3H). MS (ESI) calc'd for (C₂₄H₂₇N₅O₃) [M+H]⁺, 434.2; found, 434.5.

### 4-((S)-3-hydroxypyrrolidine-1-carbonyl)-N-(3-((R)-1-(1-methyl-1H-imidazol-2-yl)propan-2-yl)phenyl)picolinamide (252a)

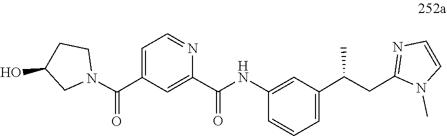 [SMILES: C[C@H](Cc1nccn1C)c1cccc(NC(=O)c2cc(C(=O)N3CC[C@H](O)C3)ccn2)c1]

The racemic 252 (50 mg) was separated by prep chiral-HPLC to afford 4-((S)-3-hydroxypyrrolidine-1-carbonyl)-N-(3-(R)-1-(1-methyl-1H-imidazol-2-yl)propan-2-yl)phenyl)picolinamide (17.9 mg) and 4-(S)-3-hydroxypyrrolidine-1-carbonyl)-N-(3-((S)-1-(1-methyl-1H-imidazol-2-yl)propan-2-yl)phenyl)picolinamide (11 mg), ¹H NMR (300 MHz, Chloroform-d) δ 9.96 (s, 1H), 8.84-8.61 (m, 1H), 8.48-8.29 (m, 1H), 7.67-7.62 (m, 3H), 7.33-7.30 (m, 1H), 7.03-6.91 (m, 2H), 6.73 (s, 1H), 4.64-4.54 (m, 1H), 3.97-3.56 (m, 3H), 3.53-3.34 (m, 2H), 3.32 (s, 3H), 2.99-2.91 (m, 2H), 2.11-2.03 (m, 2H), 1.42 (d, J=6.9 Hz, 3H). MS (ESI) calc'd for (C₂₄H₂₇N₅O₃) [M+H]⁺, 434.2; found, 434.5.

### Example 253: 4-((2R,4S)-4-hydroxy-2-methylpyrrolidine-1-carbonyl)-N-(3-((R)-1-(1-methyl-1H-imidazol-2-yl)propan-2-yl)phenyl)picolinamide (253)

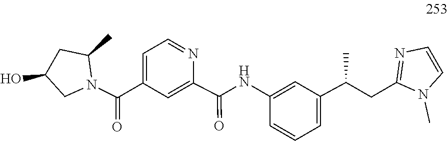 [SMILES: C[C@H](Cc1nccn1C)c1cccc(NC(=O)c2cc(C(=O)N3C[C@@H](O)C[C@H]3C)ccn2)c1]

Compound 253 (5.2 mg) was obtained according to procedures disclosed herein as a colorless solid. ¹H NMR (400 MHz, Chloroform-d) δ 9.94 (s, 1H), 8.71 (d, J=4.8 Hz, 1H), 8.34 (s, 1H), 7.64-7.58 (m, 3H), 7.32-7.28 (m, 1H), 7.06-6.84 (m, 2H), 6.71 (d, J=1.2 Hz, 1H), 4.46-4.41 (m, 2H), 4.06-4.02 (m, 1H), 3.60-3.56 (m, 2H), 3.38-3.35 (m, 1H), 3.30 (s, 3H), 3.07-3.01 (m, 1H), 2.97-2.91 (m, 1H), 2.47-2.40 (m, 1H), 1.89-1.67 (m, 1H), 1.54 (d, J=6.4 Hz, 3H), 1.42 (d, J=6.9 Hz, 3H), 1.17 (d, J=6.4 Hz, 1H). MS (ESI) calc'd for (C₂₅H₂₉N₅O₃) [M+H]⁺, 448.2; found, 448.5.

### Example 254: 2-fluoro-5-((S)-3-hydroxypyrrolidine-1-carbonyl)-N-(3-((R)-1-(4-methyl-4H-1,2,4-triazol-3-yl)propan-2-yl)phenyl)benzamide (254)

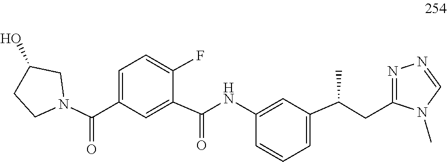 [SMILES: C[C@H](Cc1nncn1C)c1cccc(NC(=O)c2cc(C(=O)N3CC[C@H](O)C3)ccc2F)c1]

Compound 254 (48 mg, 11%) was obtained according to procedures disclosed herein. ¹H NMR (400 MHz, DMSO-d₆) δ 10.44 (s, 1H), 8.40 (s, 1H), 7.79-7.71 (m, 2H), 7.61-7.56 (m, 2H), 7.44-7.39 (m, 1H), 7.29-7.25 (m, 1H), 7.03 (d, J=7.6 Hz, 1H), 5.00 (s, 1H), 4.39-4.19 (m, 1H), 3.64-3.50 (m, 3H), 3.48-3.37 (m, 3H), 3.27-3.21 (m, 2H), 2.99 (d, J=7.2 Hz, 2H), 1.99-1.89 (m, 1H), 1.88-1.78 (m, 1H), 1.28 (d, J=6.8 Hz, 3H). MS (ESI) calculated for (C₂₄H₂₆FN₅O₃) [M+H]⁺, 452.2; found, 452.0.

### Example 255: (R)-2-(3-(1-(4-methyl-4H-1,2,4-triazol-3-yl)propan-2-yl)phenyl)-4-(trifluoromethyl)isoindolin-1-one (255a)

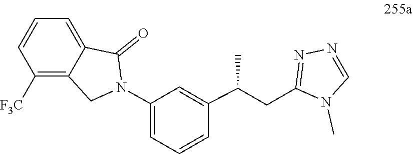 [SMILES: C[C@H](Cc1nncn1C)c1cccc(N2Cc3c(cccc3C(F)(F)F)C2=O)c1]

In a 250 mL round bottom flask was added D-a (9.00 g, 41.6 mmol, 1 eq.) and methyl 2-(bromomethyl)-3-(trifluoromethyl)benzoate (13.6 g, 45.8 mmol, 1.1 eq.). Methanol (40 mL) was added at rt and stirred for several minutes to completely dissolve the substrates. Silver nitrate (9.19 g, 54.1 mmol, 1.3 eq.) in water (10 mL) was added dropwise to the reaction with a slight exotherm. After the addition, the reaction was stirred for five minutes. The reaction was stirred at 60° C. overnight. The mixture was cooled to rt, and 1 N HCl (40 mL) was added. The reaction was filtered through celite and rinsed with minimum amount of methanol. The solution was transferred into a separatory funnel and washed with a mixture of hexanes and EtOAc (1:1, 40 mL×3). The product was extracted using a mixture of chloroform and isopropyl alcohol (2:1, 40 mL×5). The combined organic layers were washed with water (40 mL×1). This water was back extracted with DCM (40 mL×2). The combined organic layers were filtered through a pad of celite and silica gel (50 mL) and rinsed with chloroform:isopropyl alcohol (2:1, 50 mL×6). The solvents were evaporated and dried under high vacuum. EtOAc (40 mL) was added to the crude material and the material was sonicated until solids formed. The solid was allowed to develop fully and then filtered. The obtained solid was rinsed thoroughly with EtOAc (30 mL total). After drying overnight under high vacuum, the title compound was obtained as the hydrochloride salt as an off-white solid. Yield: 11.3 g (66%). ¹H NMR (500 MHz, DMSO-d6) δ 9.08 (s, 1H), 8.06 (dd, J=19.3, 7.7 Hz, 2H), 7.88-7.71 (m, 3H), 7.38 (dd, J=8.9, 7.6 Hz, 1H), 7.13 (dt, J=7.7, 1.3 Hz, 1H), 5.20 (dd, J=3.9, 1.6 Hz, 2H), 3.65 (s, 3H), 3.35 (d, J=7.0 Hz, 1H), 3.30-3.18 (m, 2H), 1.35 (d, J=6.8 Hz, 3H). LCMS: C₂₁H₁₉F₃N₄O requires: 400.4, found: m/z 401.4 [M+H]+.

### Example 256: (R)-4-bromo-2-(3-(1-(4-methyl-4H-1,2,4-triazol-3-yl)propan-2-yl)phenyl)isoindolin-1-one (256)

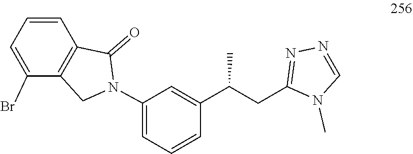 [SMILES: C[C@H](Cc1nncn1C)c1cccc(N2Cc3c(Br)cccc3C2=O)c1]

A solution of D-a (470 mg, 2.2 mmol, 1 eq), methyl 3-bromo-2-(bromomethyl)benzoate (700 mg, 2.2 mmol, 1 eq), triethylamine (0.32 mL, 2.3 mmol, 1.1 eq) and ethanol (4 mL) was heated at 60° C. for 16 h. The solution was partitioned between saturated aqueous ammonium chloride (25 mL) and extracted with EtOAc (3×10 mL). The combined organic phases were dried (sodium sulfate), filtered and concentrated onto Celite. The title compound (550 mg) was obtained as a colorless solid using standard chromatography purification methods. ¹H NMR (500 MHz, DMSO-d₆) δ 8.88 (s, 1H), 7.91 (dd, J=7.9, 0.9 Hz, 1H), 7.86-7.72 (m, 3H), 7.54 (t, J=121 Hz, 1H), 7.37 (t, J=7.8 Hz, 1H), 7.12 (dt, J=7.8, 1.2 Hz, 1H), 5.03-4.88 (m, 2H), 3.61 (s, 3H), 3.35 (h, J=6.9 Hz, 1H), 3.19 (dd, J=7.6, 1.8 Hz, 2H), 1.35 (d, J=6.9 Hz, 3H); LCMS: C₂₀H₁₉BrN₄O requires: 410, found: m/z=411 [M+H]⁺.

### Example 257: 4-cyclopropyl-2-[3-[(1R)-1-methyl-2-(4-methyl-1,2,4-triazol-3-yl)ethyl]phenyl]-3H-pyrrolo[3,4-c]pyridin-1-one (257)

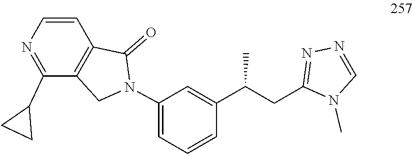 [SMILES: C[C@H](Cc1nncn1C)c1cccc(N2Cc3c(ccnc3C3CC3)C2=O)c1]

Step 1: Methyl 2-cyclopropyl-3-methyl-pyridine-4-carboxylate. To a stirred solution of methyl 2-chloro-3-methyl-pyridine-4-carboxylate (1 g, 5.39 mmol, 1 eq.) and tetrakis(triphenylphosphine)palladium(0) (0.62 g, 0.54 mmol, 0.1 eq.) in THF (55 mL) was added bromo(cyclopropyl)zinc (12.3 mL, 6.2 mmol, 1.2 eq.). The solution was heated at about 60° C. for 14 h. The reaction was quenched with saturated aqueous ammonium chloride. After General Work-up Procedure 1, The mixture was purified by flash column chromatography eluting with 0-50% EtOAc in hexanes to afford the title compound (560 mg, 2.93 mmol, 54% yield).

Step 2: Methyl 3-(bromomethyl)-2-cyclopropyl-pyridine-4-carboxylate. To a stirred solution of methyl 2-cyclopropyl-3-methyl-pyridine-4-carboxylate (700 mg, 3.66 mmol, 1 eq.) and N-bromosuccinimide (717 mg, 4.03 mmol, 1.1 eq.) in carbontetrachloride (35 mL) was added benzoyl peroxide (177 mg, 0.73 mmol, 0.2 eq.). The mixture was stirred at 80° C. for about 14 h. The mixture was concentrated then purified by flash column chromatography eluting with 0-50% EtOAc in petroleum ether to afford the title compound (632 mg, 2.34 mmol, 64% yield).

Step 3: 4-cyclopropyl-2-[3-[(1R)-1-methyl-2-(4-methyl-1,2,4-triazol-3-yl)ethyl]phenyl]-3H-pyrrolo[3,4-c]pyridin-1-one To a stirred solution of methyl 3-(bromomethyl)-2-cyclopropyl-pyridine-4-carboxylate (0.26 g, 0.98 mmol, 1 eq.) and D-a (0.21 g, 0.98 mmol, 1 eq.) in dimethylsulfoxide (9.7 mL) was added and triethylamine (0.14 mL, 1.03 mmol, 1.05 eq.). The mixture was heated at about 60° C. for about 14 h. The mixture was cooled to about 25° C., concentrated, and residue was purified by flash column chromatography to afford the title compound (32 mg, 0.09 mmol, 8.7% yield). ¹H NMR (500 MHz, DMSO-d6) δ 8.61 (d, J=4.9 Hz, 1H), 8.29 (s, 1H), 7.87 (ddd, J=8.2, 2.3, 1.0 Hz, 1H), 7.83 (t, J=1.9 Hz, 1H), 7.49 (d, J=4.9 Hz, 1H), 7.38 (t, J=7.9 Hz, 1H), 7.13 (dt, J=7.8, 1.3 Hz, 1H), 5.21 (s, 2H), 3.46 (s, 3H), 3.17 (d, J=5.2 Hz, 1H), 3.02 (dd, J=7.4, 2.7 Hz, 2H), 2.26 (tt, J=7.9, 4.8 Hz, 1H), 1.32 (d, J=6.9 Hz, 3H), 1.17-1.06 (m, 4H). LCMS: C₂₂H₂₃N₅O requires: 373.2, found: m/z=374.4 [M+H]⁺.

### Example 258: (R)-4-cyclobutyl-2-(3-(1-(4-methyl-4H-1,2,4-triazol-3-yl)propan-2-yl)phenyl)-2,3-dihydropyrrolo[3,4-c]pyridin-1-one (258)

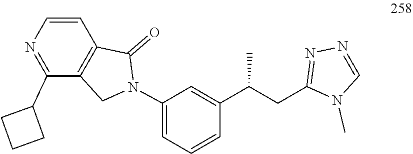 [SMILES: C[C@H](Cc1nncn1C)c1cccc(N2Cc3c(ccnc3C3CCC3)C2=O)c1]

Step 1: Synthesis of methyl 3-(bromomethyl)-2-chloroisonicotinate. The radical bromination reaction was performed in a similar fashion to Example 257 Step 2 to afford methyl 3-(bromomethyl)-2-chloroisonicotinate (930 mg, 89%) as a yellow oil. MS (ESI) calc'd for (C₈H₇BrClNO₂) [M+H]⁺, 263.9; found 263.9.

Step 2: Synthesis of (R)-4-chloro-2-(3-(1-(4-methyl-4H-1,2,4-triazol-3-yl)propan-2-yl)phenyl)-2,3-dihydropyrrolo[3,4-c]pyridin-1-one. Using methyl 3-(bromomethyl)-2-chloroisonicotinate (300 mg, 1.13 mmol) and D-a (245 mg, 1.13 mmol) the reaction was performed in a similar fashion to Example 256 to afford the title compound (360 mg, 86%) as a yellow solid, which was used without purification. MS (ESI) calc'd for (C₁₉H₁₈ClN₅O) [M+H]⁺, 368.1; found, 368.1.

Step 3: Synthesis of (R)-4-cyclobutyl-2-(3-(1-(4-methyl-4H-1,2,4-triazol-3-yl)propan-2-yl)phenyl)-2,3-dihydropyrrolo[3,4-c]pyridin-1-one. To a mixture of (R)-4-chloro-2-(3-(1-(4-methyl-4H-1,2,4-triazol-3-yl)propan-2-yl)phenyl)-2,3-dihydropyrrolo[3,4-c]pyridin-1-one (190 mg, 0.52 mmol), Pd(dppf)Cl₂ (20 mg, 0.03 mmol) in THF (0.3 mL) and toluene (1 mL) was added bromo(cyclobutyl)zinc (0.5 M in THF, 1.6 mL, 0.80 mmol) at 80° C. The mixture was stirred at 90° C. for 16 h under nitrogen. The reaction was then quenched by the addition of saturated ammonium chloride solution, followed by General Work-up Procedure 1. The residue was purified by chromatography C to the title compound (23.4 mg, 11%) as an off-white solid. 1H NMR (300 MHz, DMSO-d₆) δ 8.76 (d, J=4.8 Hz, 1H), 8.30 (s, 1H), 7.91-7.76 (m, 2H), 7.58 (d, J=4.8 Hz, 1H), 7.37 (t, J=7.8 Hz, 1H), 7.13 (d, J=7.8 Hz, 1H), 5.06 (s, 2H), 4.04-3.79 (m, 1H), 3.46 (s, 3H), 3.30-3.28 m, 1H), 3.03-3.00 (m, 2H), 2.49-2.44 (m, 2H), 2.41-2.30 (m, 2H), 2.15-2.02 (m, 1H), 1.96-1.90 (m, 1H), 1.32 (d, J=6.9 Hz, 3H). MS (ESI) calc'd for (C₂₃H₂₅N₅O) [M+H]⁺, 388.2; found, 388.2.

### Example 259: (R)-4-isopropyl-2-(3-(1-(4-methyl-4H-1,2,4-triazol-3-yl)propan-2-yl)phenyl)-2,3-dihydro-1H-pyrrolo[3,4-c]pyridin-1-one (259)

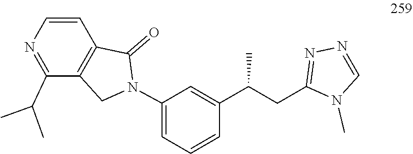 [SMILES: CC(C)c1nccc2c1CN(c1cccc([C@H](C)Cc3nncn3C)c1)C2=O]

Step 1: Synthesis of (R)-2-(3-(1-(4-methyl-4H-1,2,4-triazol-3-yl)propan-2-yl)phenyl)-4-(prop-1-en-2-yl)-2,3-dihydropyrrolo[3,4-c]pyridin-1-one. To a solution of (R)-4-chloro-2-(3-(1-(4-methyl-4H-1,2,4-triazol-3-yl)propan-2-yl)phenyl)-2,3-dihydropyrrolo[3,4-c]pyridin-1-one (step 3, synthesis 258) (1.0 g, 2.72 mmol) in THF (10 mL) and water (10 mL) were added potassium trifluoro(prop-1-en-2-yl)-λ4-borane (0.8 g, 5.43 mmol), Pd(dppf)Cl₂ (200 mg, 0.27 mmol) and cesium carbonate (2.7 g, 8.15 mmol). The mixture was stirred at 100° C. for 16 h under nitrogen and then filtered. The filtrate was concentrated. The residue was purified by reverse phase flash column chromatography with 5-40% acetonitrile in water to afford the title compound (400 mg, 39%) as a colorless solid. MS (ESI) calc'd for (C₂₂H₂₃N₅O) [M+H]⁺, 374.2; found, 374.2.

Step 2: Synthesis of (R)-4-isopropyl-2-(3-(1-(4-methyl-4H-1,2,4-triazol-3-yl)propan-2-yl)phenyl)-2,3-dihydropyrrolo[3,4-c]pyridin-1-one. To a solution of (R)-2-(3-(1-(4-methyl-4H-1,2,4-triazol-3-yl)propan-2-yl)phenyl)-4-(prop-1-en-2-yl)-2,3-dihydropyrrolo[3,4-c]pyridin-1-one (40 mg, 0.11 mmol) in methanol (0.5 mL) was added palladium on carbon (10%, 10 mg). The mixture was stirred at rt for 3 h under hydrogen and then filtered. The filtrate was purified by Prep-HPLC to afford the title compound (20.1 mg, 50%) as an off-white solid, ¹H NMR (300 MHz, DMSO-d₆) δ 8.73 (d, J=4.8 Hz, 1H), 8.37 (s, 1H), 8.00-7.74 (m, 2H), 7.59 (d, J=4.8 Hz, 1H), 7.38 (t, J=7.8 Hz, 1H), 7.13 (d, J=7.8 Hz, 1H), 5.16 (s, 2H), 3.47 (s, 3H), 3.36-3.26 (m, 2H), 3.06-3.03 (m, 2H), 1.33 (d, J=6.8 Hz, 9H). MS (ESI) calc'd for (C₂₂H₂₅N₅O) [M+H]⁺, 376.3; found 376.3.

### Example 260: (R)-6-cyclopropyl-2-(3-(1-(4-methyl-4H-1,2,4-triazol-3-yl)propan-2-yl)phenyl)-4-(trifluoromethyl)isoindolin-1-one (260)

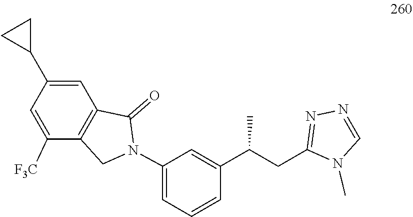 [SMILES: C[C@H](Cc1nncn1C)c1cccc(N2Cc3c(cc(C4CC4)cc3C(F)(F)F)C2=O)c1]

Step 1: Synthesis of methyl 5-bromo-2-(bromomethyl)-3-(trifluoromethyl)benzoate. Following the procedure in Example 257 Step 2 the reaction was performed to give the title compound (402 mg) which was used without purification.

Step 2: Synthesis of (R)-6-bromo-2-(3-(1-(4-methyl-4H-1,2,4-triazol-3-yl)propan-2-yl)phenyl)-4-(trifluoromethyl)isoindolin-1-one. Using D-a (138 mg, 0.638 mmol) (182 mg, 0.843 mmol) and methyl 5-bromo-2-(bromomethyl)-3-(trifluoromethyl)benzoate (398 mg, 1.06 mmol) the reaction was performed in a similar fashion to Example 255 to give the title compound (250 mg, 52% yield).

Step 3: Synthesis of (R)-6-cyclopropyl-2-(3-(1-(4-methyl-4H-1,2,4-triazol-3-yl)propan-2-yl)phenyl)-4-(trifluoromethyl)isoindolin-1-one. (R)-6-bromo-2-(3-(1-(4-methyl-4H-1,2,4-triazol-3-yl)propan-2-yl)phenyl)-4-(trifluoromethyl)isoindolin-1-one (61 mg, 0.13 mmol), potassium phosphate hydrate (140 mg, 0.66 mmol), cyclopropyl boronic acid (17 mg, 0.20 mmol) and tricyclohexylphosphine (3.8 mg, 0.014 mmol) were suspended in a mixture of toluene (1 mL) and water (0.1 mL). The reaction was purged with nitrogen. Palladium acetate (1.5 mg, 0.0067 mmol) was added and the reaction was stirred at 100° C. for 2 h and then at 80° C. overnight. The title compound (21 mg, 31% yield) was obtained using standard chromatography purification methods, ¹H NMR (500 MHz, DMSO-d₆) δ 8.86 (s, 1H), 7.88-7.61 (m, 4H), 7.36 (t, J=7.9 Hz, 1H), 7.16-7.06 (m, 1H), 5.19-5.04 (m, 2H), 3.59 (s, 3H), 3.33 (q, J=7.9 Hz, 1H), 3.18 (dd, J=7.6, 1.7 Hz, 2H), 2.23 (tt, J=8.5, 5.0 Hz, 1H), 1.33 (d, J=6.9 Hz, 3H), 1.15-1.02 (m, 2H), 0.86 (dt, J=6.7, 4.6 Hz, 2H). LCMS: C₂₄H₂₃F₃N₄O requires: 440.5, found: m/z 441.5 [M+H]⁺.

### Example 261: (R)-2-(3-(1-(oxazol-2-yl)propan-2-yl)phenyl)-4-(trifluoromethyl)isoindolin-1-one (261)

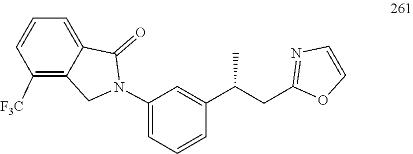 [SMILES: C[C@H](Cc1ncco1)c1cccc(N2Cc3c(cccc3C(F)(F)F)C2=O)c1]

Compound 261 (187.8 mg, 49%) was obtained according to procedures disclosed herein as a colorless solid. ¹H NMR (300 MHz, DMSO-d₆) δ 8.13-8.03 (m, 2H), 7.97 (s, 1H), 7.82-7.77 (m, 3H), 7.37 (t, J=7.8 Hz, 1H), 7.14-7.08 (m, 2H), 5.22 (s, 2H), 3.39-3.25 (m, 1H), 3.08 (d, J=7.5 Hz, 2H), 1.28 (d, J=6.9 Hz, 3H). MS (ESI) calc'd for (C₂₁H₁₇F₃N₂O₂) [M+H]⁺, 387.1; found, 387.1.

### Example 262: (R)-2-(3-(1-(4H-1,2,4-triazol-3-yl)propan-2-yl)phenyl)-4-(trifluoromethyl)isoindolin-1-one (262)

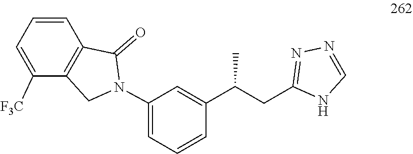 [SMILES: C[C@H](Cc1nnc[nH]1)c1cccc(N2Cc3c(cccc3C(F)(F)F)C2=O)c1]

Using E (200 mg, 0.99 mmol) and methyl 2-(bromomethyl)-3-(trifluoromethyl)benzoate (294 mg, 0.99 mmol) the reaction was performed in a similar fashion to Example 256 to afford the title compound (103.6 mg, 27%) as a colorless solid. 1H NMR (400 MHz, DMSO-d₆) δ 13.61 (s, 1H), 8.09-8.03 (m, 2H), 7.84-7.75 (m, 4H), 7.38-7.34 (m, 1H), 7.11 (t, J=8.0 Hz, 1H), 5.22 (s, 2H), 3.30-3.28 (m, 1H), 3.07-2.86 (m, 2H), 1.25-1.21 (m, 3H). MS (ESI) calc'd for (C₂₀H₁₇F₃N₄O) [M+H]⁺, 387.1; found, 387.1.

### Example 263 and Example 264: (R)-2-(3-(1-(1-methyl-1H-1,2,4-triazol-3-yl)propan-2-yl)phenyl)-4-(trifluoromethyl)isoindolin-1-one (263) and (R)-2-(3-(1-(2-methyl-2H-1,2,4-triazol-3-yl)propan-2-yl)phenyl)-4-(trifluoromethyl)isoindolin-1-one (264)

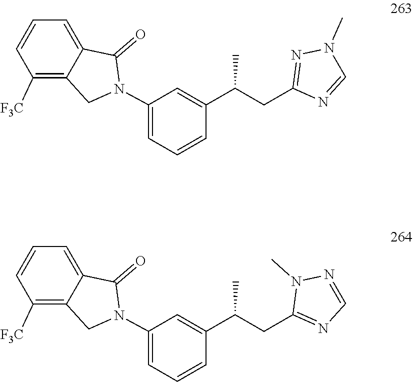 [SMILES: C[C@H](Cc1ncn(C)n1)c1cccc(N2Cc3c(cccc3C(F)(F)F)C2=O)c1 ; C[C@H](Cc1ncnn1C)c1cccc(N2Cc3c(cccc3C(F)(F)F)C2=O)c1]

Compound 263 (59.2 mg, 13%) was obtained as a light yellow oil and Compound 264 (76.6 mg, 16%) was obtained as a colorless solid according to procedures disclosed herein.

(R)-2-(3-(1-(1-methyl-1H-1,2,4-triazol-3-yl)propan-2-yl)phenyl)-4-(trifluoromethyl)isoindolin-1-one: ¹H NMR (300 MHz, DMSO-d₆) δ 8.32 (s, 1H), 8.10-8.02 (m, 2H), 7.83-7.76 (m, 3H), 7.37 (t, J=7.8 Hz, 1H), 7.14-7.11 (m, 1H), 5.22 (s, 2H), 3.78 (s, 3H), 3.27-3.21 (m, 1H), 2.95-2.81 (m, 2H), 1.24 (d, J=6.9 Hz, 3H). MS (ESI) calc'd for (C₂₁H₁₉F₃N₄O) [M+H]⁺, 401.1; found, 401.1.

(R)-2-(3-(1-(2-methyl-2H-1,2,4-triazol-3-yl)propan-2-yl)phenyl)-4-(trifluoromethyl)isoindolin-1-one: ¹H NMR (300 MHz, DMSO-d₆) δ 8.10-8.03 (m, 2H), 7.82-7.77 (m, 4H), 7.35 (t, J=7.8 Hz, 1H), 7.09 (d, J=7.8 Hz, 1H), 5.20 (s, 2H), 3.62 (s, 3H), 3.29-3.27 (m, 1H), 3.05 (d, J=7.5 Hz, 2H), 1.24 (d, J=6.9 Hz, 3H). MS (ESI) calc'd for (C₂₁H₁₉F₃N₄O) [M+H]⁺, 401.1; found, 401.1.

### Example 265: (R)-2-(3-(1-(4-cyclopropyl-4H-1,2,4-triazol-3-yl)propan-2-yl)phenyl)-4-(trifluoromethyl)isoindolin-1-one (265)

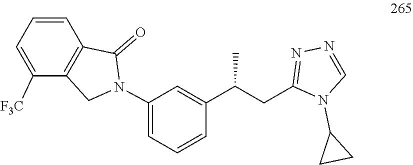 [SMILES: C[C@H](Cc1nncn1C1CC1)c1cccc(N2Cc3c(cccc3C(F)(F)F)C2=O)c1]

Step 1: Synthesis of (R)-4-cyclopropyl-3-(2-(3-nitrophenyl)propyl)-4H-1,2,4-triazole. To a solution of (R)-N,N-dimethyl-N′-(3-(3-nitrophenyl)butanoyl)formohydrazonamide (5.0 g, 18.0 mmol) in acetonitrile (72 mL) and acetic acid (18 mL) was added cyclopropanamine (5.1 g, 89.8 mmol) at 0° C. The mixture was stirred at 95° C. for 16 h. The mixture was concentrated. The residue was diluted with water. The mixture was basified by saturated sodium carbonate aqueous solution to pH 8, followed by General Work-up Procedure 1. The residue was purified by flash column chromatography with 0-8% methanol in EtOAc to afford the title compound (600 mg, 12%) as a light brown oil. MS (ESI) calc'd for (C₁₄H₁₆N₄O₂) [M+H]⁺, 273.1; found, 273.2.

Step 2: Synthesis of (R)-3-(1-(4-cyclopropyl-4H-1,2,4-triazol-3-yl)propan-2-yl)aniline. To a solution of (R)-4-cyclopropyl-3-(2-(3-nitrophenyl)propyl)-4H-1,2,4-triazole (600 mg, 2.20 mmol) in ethanol (20 mL) was added palladium (60.0 mg, wet, 5% on carbon) at rt. The flask was evacuated and backfilled with hydrogen. The mixture was stirred at rt for 16 h under hydrogen atmosphere (2 atm). The solids were filtered off. The filtrate was concentrated to afford the title compound (450 mg, 84%) as a brown oil. MS (ESI) calc'd for (C₁₄H₁₈N₄) [M+H]⁺, 243.3; found, 243.3.

Step 3: Synthesis of (R)-2-(3-(1-(4-cyclopropyl-4H-1,2,4-triazol-3-yl)propan-2-yl)phenyl)-4-(trifluoromethyl)isoindolin-1-one. Using (R)-3-(1-(4-cyclopropyl-4H-1,2,4-triazol-3-yl)propan-2-yl)aniline (200 mg, 0.83 mmol) and methyl 2-(bromomethyl)-3-(trifluoromethyl)benzoate (247 mg, 0.83 mmol) the reaction was performed in a similar fashion to Example 256 to afford the title compound (58.1 mg, 16%) as a light yellow solid. ¹H NMR (300 MHz, DMSO-d₆) δ 8.39 (s, 1H), 8.10-8.03 (m, 2H), 7.89-7.83 (m, 1H), 7.80-7.71 (m, 2H), 7.36 (t, J=7.8 Hz, 1H), 7.13 (d, J=7.8 Hz, 1H), 5.22 (s, 2H), 3.46-3.41 (m, 1H), 3.23-3.09 (m, 3H), 1.32 (d, J=6.9 Hz, 3H), 1.05-0.75 (m, 4H). MS (ESI) calc'd for (C₂₃H₂₁F₃N₄O) [M+H]⁺, 427.3; found, 427.3.

### Example 266: (R)-2-(3-(1-(1,3,4-oxadiazol-2-yl)propan-2-yl)phenyl)-4-(trifluoromethyl)isoindolin-1-one (266)

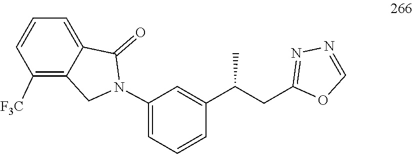 [SMILES: C[C@H](Cc1nnco1)c1cccc(N2Cc3c(cccc3C(F)(F)F)C2=O)c1]

Compound 266 (251 mg, 66%) was obtained according to procedures disclosed herein as a colorless solid. ¹H NMR (400 MHz, DMSO-d₆) δ 9.11 (s, 1H), 8.10-8.04 (m, 2H), 7.85-7.78 (m, 3H), 7.38 (t, J=8.0 Hz, 1H), 7.14 (d, J=7.6 Hz, 1H), 5.22 (s, 2H), 3.37-3.31 (m, 1H), 3.25-3.23 (m, 2H), 1.32 (d, J=6.8 Hz, 3H). MS (ESI) calc'd for (C₂₀H₁₆F₃N₃O₂) [M+H]⁺, 388.1; found, 388.2.

### Example 267: 2-(3-(1-(1-methyl-1H-imidazol-2-yl)propan-2-yl)phenyl)-4-(trifluoromethyl)isoindolin-1-one (267)

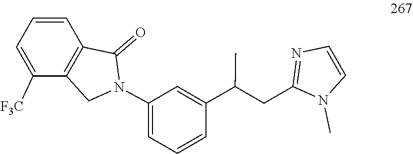 [SMILES: CC(Cc1nccn1C)c1cccc(N2Cc3c(cccc3C(F)(F)F)C2=O)c1]

Compound 267 (150 mg, 29%) was obtained according to procedures disclosed herein as a yellow oil. MS (ESI) calc'd for (C₂₂H₂₀F₃N₃O) [M+H]⁺, 400.1; found, 400.1. ¹H NMR (400 MHz, DMSO-d₆) δ 8.10-8.03 (m, 2H), 7.85-7.77 (m, 2H), 7.73 (t, J=2.0 Hz, 1H), 7.36 (t, J=8.0 Hz, 1H), 7.11 (d, J=8.0 Hz, 1H), 6.94 (d, J=0.8 Hz, 1H), 6.75 (d, J=0.8 Hz, 1H), 5.19 (s, 2H), 3.41 (s, 3H), 3.31-3.26 (m, 1H), 2.90 (d, J=7.2 Hz, 2H), 1.29 (d, J=6.8 Hz, 3H).

### (R)-2-(3-(1-(1-methyl-1H-imidazol-2-yl)propan-2-yl)phenyl)-4-(trifluoromethyl)isoindolin-1-one (267a)

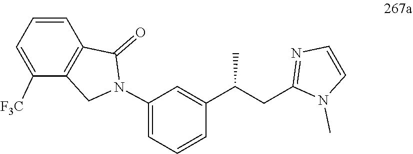 [SMILES: C[C@H](Cc1nccn1C)c1cccc(N2Cc3c(cccc3C(F)(F)F)C2=O)c1]

Compound 267a was separated by chiral HPLC. ¹H NMR (300 MHz, DMSO-d₆) δ 8.11-8.03 (m, 2H), 7.85-7.76 (m, 2H), 7.74 (s, 1H), 7.36 (t, J=7.8 Hz, 1H), 7.11 (d, J=7.8 Hz, 1H), 6.95 (d, J=1.2 Hz, 1H), 6.77 (d, J=1.2 Hz, 1H), 5.19 (s, 2H), 3.41 (s, 3H), 3.33-3.23 (m, 1H), 2.90 (d, J=13 Hz, 2H), 1.28 (d, J=6.9 Hz, 3H). MS (ESI) calc'd for (C₂₅H₂₄F₃N₅O₃) [M+H]⁺, 400.2; found, 399.9.

### Example 268: 2-(3-(1-(4-methylisoxazol-3-yl)propan-2-yl)phenyl)-4-(trifluoromethyl)isoindolin-1-one (268)

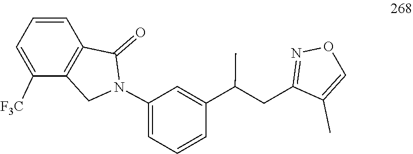 [SMILES: Cc1conc1CC(C)c1cccc(N2Cc3c(cccc3C(F)(F)F)C2=O)c1]

Synthesis of 2-(3-(1-(4-methylisoxazol-3-yl)propan-2-yl)phenyl)-4-(trifluoromethyl)isoindolin-1-one. Using G (500 mg, 2.30 mmol) and methyl 2-(bromomethyl)-3-(trifluoromethyl)benzoate (680 mg, 2.30 mmol) to the reaction was performed in a similar fashion to Example 256 to afford the title compound (400 mg, 43%). ¹H NMR (300 MHz, DMSO-d₆) δ 8.51 (d, J=0.9 Hz, 1H), 8.10-8.02 (m, 2H), 7.90-7.73 (m, 3H), 7.38 (t, J=7.8 Hz, 1H), 7.14 (d, J=7.8 Hz, 1H), 5.21 (s, 2H), 3.23-3.16 (m, 1H), 2.92 (d, J=7.2 Hz, 2H), 1.88 (s, 3H), 1.28 (d, J=6.9 Hz, 3H). MS (ESI) calc'd for (C₂₂H₁₉F₃N₂O₂) [M+H]⁺, 401.1; found, 401.0.

### Example 269: 2-(3-(1-(4-(hydroxymethyl)isoxazol-3-yl)propan-2-yl)phenyl)-4-(trifluoromethyl)isoindolin-1-one (269)

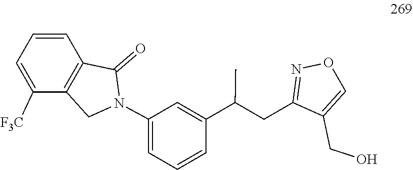 [SMILES: CC(Cc1nocc1CO)c1cccc(N2Cc3c(cccc3C(F)(F)F)C2=O)c1]

Compound 269 (300 mg, 26%) was obtained according to procedures disclosed herein as a colorless oil. ¹H NMR (300 MHz, DMSO-d₆) δ 8.63 (s, 1H), 8.10-8.02 (m, 2H), 7.90-7.73 (m, 3H), 7.36 (t, J=7.8 Hz, 1H), 7.12 (d, J=7.8 Hz, 1H), 5.22 (s, 2H), 5.10 (t, J=5.1 Hz, 1H), 4.30 (d, J=5.1 Hz, 2H), 3.32-3.20 (m, 1H), 3.08-2.88 (m, 2H), 1.29 (d, J=6.9 Hz, 3H). MS (ESI) calc'd for (C₂₂H₁₉F₃N₂O₃) [M+H]⁺, 417.1; found, 417.0.

### Example 270: (S)-2-(3-(1-(4-methyl-1H-pyrazol-3-yl)propan-2-yl)phenyl)-4-(trifluoromethyl)isoindolin-1-one (270a) and (R)-2-(3-(1-(4-methyl-1H-pyrazol-3-yl)propan-2-yl)phenyl)-4-(trifluoromethyl)isoindolin-1-one (270b)

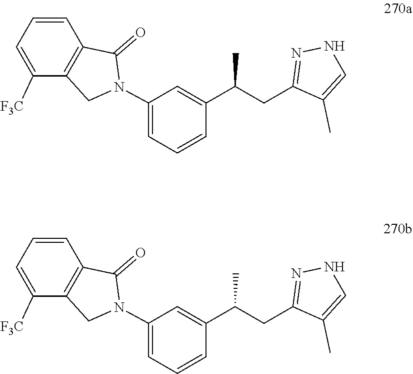 [SMILES: Cc1c[nH]nc1C[C@H](C)c1cccc(N2Cc3c(cccc3C(F)(F)F)C2=O)c1 ; Cc1c[nH]nc1C[C@@H](C)c1cccc(N2Cc3c(cccc3C(F)(F)F)C2=O)c1]

Step 1: Synthesis of 2-[3-[1-(4-methyl-1H-pyrazol-3-yl)propan-2-yl]phenyl]-4-(trifluoromethyl)isoindolin-1-one. Using F (350 mg, 1.63 mmol) and methyl 2-(bromomethyl)-3-(trifluoromethyl)benzoate (483 mg, 1.63 mmol) the reaction was performed in a similar fashion to Example 256 to afford the title compound (160 mg, 25%) as a colorless solid. MS (ESI) calc'd for (C₂₂H₂₀F₃N₃O) [M+H]⁺, 400.2; found, 400.1.

Step 2: Synthesis of (S)-2-(3-(1-(4-methyl-1H-pyrazol-3-yl)propan-2-yl)phenyl)-4-(trifluoromethyl)isoindolin-1-one and (R)-2-(3-(1-(4-methyl-1H-pyrazol-3-yl)propan-2-yl)phenyl)-4-(trifluoromethyl)isoindolin-1-one. The racemic 2-[3-[1-(4-methyl-1H-pyrazol-3-yl)propan-2-yl]phenyl]-4-(trifluoromethyl)isoindolin-1-one (140 mg) was separated by Chiral-Prep-HPLC:[Column: Chiralpak IA, 2×25 cm, 5 um; Mobile Phase A: Hex (8 mmol/L NH₃.methanol), Mobile Phase B: ethanol; Flow rate: 20 mL/min; Gradient: 20 B to 20 B in 18 min; 254/220 nm] to afford the title compounds as colorless solids:

(S)-2-(3-(1-(4-methyl-1H-pyrazol-3-yl)propan-2-yl)phenyl)-4-(trifluoromethyl)isoindolin-1-one: First eluting product, 40 mg: MS (ESI) calc'd for (C₂₂H₂₀F₃N₃O) [M+H]⁺, 400.2; found, 400.1. ¹H NMR (400 MHz, DMSO-d₆) δ 12.20 (s, 1H), 8.10 (d, J=7.8 Hz, 1H), 8.05 (d, J=7.8 Hz, 1H), 7.87-7.69 (m, 3H), 7.36 (t, J=7.8 Hz, 1H), 7.22 (s, 1H), 7.10 (d, J=7.8 Hz, 1H), 5.21 (s, 2H), 3.16-3.09 (m, 1H), 2.86-2.74 (m, 2H), 1.87 (s, 3H), 1.22 (d, J=6.9 Hz, 3H).

(R)-2-(3-(1-(4-methyl-1H-pyrazol-3-yl)propan-2-yl)phenyl)-4-(trifluoromethyl)isoindolin-1-one Later eluting product, 36 mg:MS (ESI) calc'd for (C₂₂H₂₀F₃N₃O) [M+H]⁺, 400.2; found, 400.1. ¹H NMR (400 MHz, DMSO-d₆) δ 12.24 (s, 1H), 8.10 (d, J=7.8 Hz, 1H), 8.05 (d, J=7.8 Hz, 1H), 7.85-7.71 (m, 3H), 7.36 (t, J=7.8 Hz, 1H), 7.22 (s, 1H), 7.10 (d, J=7.8 Hz, 1H), 5.21 (s, 2H), 3.16-3.09 (m, 1H), 2.86-2.74 (m, 2H), 1.87 (s, 3H), 1.22 (d, J=6.9 Hz, 3H).

### Example 271: 2-(3-(1-(4-(hydroxymethyl)-1H-pyrazol-3-yl)propan-2-yl)phenyl)-4-(trifluoromethyl)isoindolin-1-one (271)

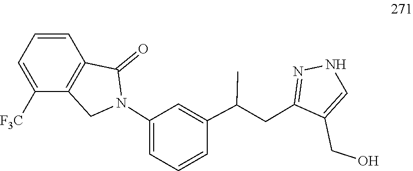 [SMILES: CC(Cc1n[nH]cc1CO)c1cccc(N2Cc3c(cccc3C(F)(F)F)C2=O)c1]

Compound 271 (60 mg, 17%) was obtained according to procedures disclosed herein as a colorless solid. ¹H NMR (400 MHz, DMSO-d₆) δ 12.32 (s, 1H), 8.10 (d, J=7.8 Hz, 1H), 8.05 (d, J=7.8 Hz, 1H), 7.85-7.72 (m, 3H), 7.48-7.29 (m, 2H), 7.10 (d, J=7.8 Hz, 1H), 5.22 (s, 2H), 4.62 (t, J=5.4 Hz, 1H), 4.26 (d, J=5.4 Hz, 2H), 3.21-3.15 (m, 1H), 2.92-2.79 (m, 2H), 1.21 (d, J=6.9 Hz, 3H). MS (ESI) calc'd for (C₂₂H₂₀F₃N₃O₂) [M+H]⁺, 416.2; found, 416.1.

### (S)-2-(3-(1-(4-(hydroxymethyl)-1H-pyrazol-3-yl)propan-2-yl)phenyl)-4-(trifluoromethyl)isoindolin-1-one (271a) and (R)-2-(3-(1-(4-(hydroxymethyl)-1H-pyrazol-3-yl)propan-2-yl)phenyl)-4-(trifluoromethyl)isoindolin-1-one (271b)

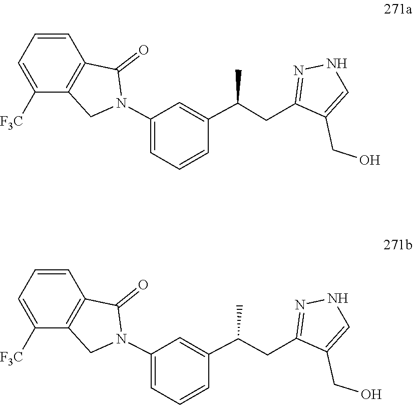 [SMILES: C[C@@H](Cc1n[nH]cc1CO)c1cccc(N2Cc3c(cccc3C(F)(F)F)C2=O)c1 ; C[C@H](Cc1n[nH]cc1CO)c1cccc(N2Cc3c(cccc3C(F)(F)F)C2=O)c1]

Compounds 271a and 271b were separated via chiral-HPLC.

(S)-2-(3-(1-(4-(hydroxymethyl)-1H-pyrazol-3-yl)propan-2-yl)phenyl)-4-(trifluoromethyl)isoindolin-1-one: MS (ESI) calc'd for (C₂₂H₂₀F₃N₃O₂) [M+H]⁺, 416.2; found, 416.1. ¹H NMR (400 MHz, DMSO-d₆) δ 12.32 (s, 1H), 8.10 (d, J=7.8 Hz, 1H), 8.05 (d, J=7.8 Hz, 1H), 7.85-7.72 (m, 3H), 7.48-7.29 (m, 2H), 7.10 (d, J=7.8 Hz, 1H), 5.22 (s, 2H), 4.62 (t, J=5.4 Hz, 1H), 4.26 (d, J=5.4 Hz, 2H), 3.21-3.15 (m, 1H), 2.92-2.79 (m, 2H), 1.21 (d, J=6.9 Hz, 3H).

(R)-2-(3-(1-(4-(hydroxymethyl)-1H-pyrazol-3-yl)propan-2-yl)phenyl)-4-(trifluoromethyl)isoindolin-1-one: MS (ESI) calc'd for (C₂₂H₂₀F₃N₃O₂) [M+H]⁺, 416.2; found, 416.1. ¹H NMR (400 MHz, DMSO-d₆) δ 12.39 (s, 1H), 8.10 (d, J=7.8 Hz, 1H), 8.05 (d, J=7.8 Hz, 1H), 7.85-7.72 (m, 3H), 7.48-7.28 (m, 2H), 7.10 (d, J=7.8 Hz, 1H), 5.22 (s, 2H), 4.62 (t, J=5 A Hz, 1H), 4.26 (d, J=5.4 Hz, 2H), 3.21-3.15 (m, 1H), 2.92-2.79 (m, 2H), 1.21 (d, J=6.9 Hz, 3H).

### Example 272: Synthesis of 2-[3-[1-(5-methyl-1H-imidazol-4-yl)propan-2-yl]phenyl]-4-(trifluoromethyl)isoindolin-1-one (272)

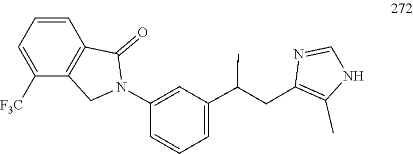 [SMILES: Cc1[nH]cnc1CC(C)c1cccc(N2Cc3c(cccc3C(F)(F)F)C2=O)c1]

Step 1: Synthesis of (4E)-5-(3-nitrophenyl)hex-4-ene-2,3-dion. A solution of (E)-3-(3-nitrophenyl)but-2-enoyl chloride (WO2010047372) (48.3 mmol, 1 eq) and anhydrous toluene (20 mL) were added to a degassed mixture of tributyl(l-ethoxyethenyl)stannane (20.9 g, 57.92 mol) and Pd(PPh₃)₂Cl₂ (3.4 g, 4.83 mmol) in anhydrous toluene (150 mL). The mixture was heated at 110° C. for 4 h under nitrogen atmosphere. The mixture was concentrated. The residue was dissolved in acetone and hydrochloric acid (aqueous, 4 N) was added thereto. The mixture was heated at 60° C. for 2 h, followed by General Work-up Procedure 1, the residue was purified by flash column chromatography with 0-25% EtOAc in petroleum ether to afford the title compound (700 mg, 6%) as a yellow semi-solid. MS (ESI) calc'd for (C₁₂H₁₁NO₄) [M+H]⁺, 234.1; found, 234.0.

Step 2: Synthesis of 5-methyl-4-[(1E)-2-(3-nitrophenyl)prop-1-en-1-yl]-1H-imidazole. A mixture of (4E)-5-(3-nitrophenyl)hex-4-ene-2,3-dione (700 mg, 3.00 mol), ammonium acetate (640 mg, 8.30 mmol), paraformaldehyde (160 mg) and acetic acid (1 mL) in ethanol (10 mL) was heated at reflux for 16 h. The mixture was diluted with sat. NaHCO₃, followed by General Work-up Procedure 1. The residue was purified by flash column chromatography with 0-10% methanol in DCM to afford the title compound (98 mg, 13%) as a yellow solid. MS (ESI) calc'd for (C₁₃H₁₃N₃O₂) [M+H]⁺, 244.1; found, 244.1.

Step 3: Synthesis of 3-[1-(5-methyl-1H-imidazol-4-yl)propan-2-yl]aniline. To a solution of 5-methyl-4-[(1E)-2-(3-nitrophenyl)prop-1-en-1-yl]-1H-imidazole (98 mg, 0.40 mmol) in methanol (10 mL) was added palladium on carbon (10%, 30 mg). The mixture was stirred at rt for 16 h under hydrogen atmosphere. The solids were filtered off. The filtrate was concentrated to afford the title compound (60 mg, crude) as a yellow solid, which was used without purification. MS (ESI) calc'd for (C₁₃H₁₇N₃) [M+H]⁺, 216.1; found, 216.2.

Step 4: Synthesis of 2-[3-[1-(5-methyl-1H-imidazol-4-yl)propan-2-yl]phenyl]-4-(trifluoromethyl)isoindolin-1-one. Using 3-[1-(5-methyl-1H-imidazol-4-yl)propan-2-yl]aniline (60 mg, crude) and methyl 2-(bromomethyl)-3-(trifluoromethyl)benzoate (82.8 mg, 0.28 mmol) to the reaction was performed in a similar fashion to Example 256 to afford the title compound (19.5 mg, 17%) as a light brown solid. ¹H NMR (300 MHz, DMSO-d₆) δ 8.09-8.02 (m, 2H), 7.82-7.72 (m, 3H), 7.53-7.51 (m, 1H), 7.34 (t, J=7.8 Hz, 1H), 7.07 (d, J=7.8 Hz, 1H), peak 5.2 ppm is missing 3.11-3.04 (m, 1H), 2.878-2.64 (m, 2H), 1.94 (s, 3H), 1.20 (d, J=6.9 Hz, 3H). MS (ESI) calc'd for (C₂₂H₂₀F₃N₃O) [M+H]⁺, 400.2; found, 400.1.

### 2-[3-[(2R)-1-(5-methyl-1H-imidazol-4-yl)propan-2-yl]phenyl]-4-(trifluoromethyl)isoindolin-1-one (272a)

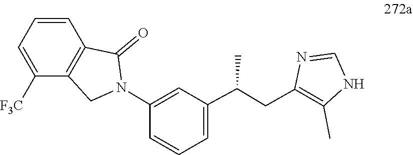 [SMILES: Cc1[nH]cnc1C[C@@H](C)c1cccc(N2Cc3c(cccc3C(F)(F)F)C2=O)c1]

The racemic 2-[3-[1-(5-methyl-1H-imidazol-4-yl)propan-2-yl]phenyl]-4-(trifluoromethyl)isoindolin-1-one (15 mg) was separated by chiral-prep-HPLC with the following conditions: [Column: CHIRAL ART Cellulose-SB, 2×25 cm, 5 um; Mobile Phase A: MTBE (0.1% DEA)-HPLC, Mobile Phase B: ethanol-HPLC; Flow rate: 20 mL/min; Gradient: 5 B to 5 B in 14 min; 220/254 nm] to afford 2-[3-[(2S)-1-(5-methyl-1H-imidazol-4-yl)propan-2-yl]phenyl]-4-(trifluoromethyl)isoindolin-1-one (2.1 mg) as a colorless solid with shorter retention time on chiral-HPLC and 2-[3-[(2R)-1-(5-methyl-1H-imidazol-4-yl)propan-2-yl]phenyl]-4-(trifluoromethyl)isoindolin-1-one (1.7 mg) as a colorless solid with longer retention time on chiral-HPLC. ¹H NMR (300 MHz, DMSO-d₆) δ 11.79 (br, 1H), 8.10-8.02 (m, 2H), 7.82-7.72 (m, 3H), 7.40-7.32 (m, 2H), 7.06 (d, J=7.8 Hz, 1H), 5.19 (s, 2H), 3.11-3.04 (m, 1H), 2.91-2.71 (m, 2H), 1.93 (s, 3H), 1.18 (d, J=6.9 Hz, 3H). MS (ESI) calc'd for (C₂₂H₂₀F₃N₃O) [M+H]⁺, 400.2; found, 400.1.

### Example 273: (R)-2-(3-(1-(4-methyl-4H-1,2,4-triazol-3-yl)propan-2-yl)phenyl)-3-oxo-7-(trifluoromethyl)isoindoline-5-carboxylic acid (273)

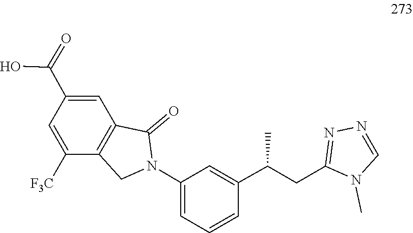 [SMILES: C[C@H](Cc1nncn1C)c1cccc(N2Cc3c(cc(C(=O)O)cc3C(F)(F)F)C2=O)c1]

Compound 273 (58 mg, 20% yield) was obtained according to procedures disclosed herein. ¹H NMR (500 MHz, DMSO-d₆) δ 13.83 (s, 1H), 8.66 (s, 1H), 8.45 (d, J=1.3 Hz, 1H), 8.41 (d, J=1.4 Hz, 1H), 7.82 (t, J=1.9 Hz, 1H), 7.82-7.76 (m, 1H), 7.38 (t, J=7.9 Hz, 1H), 7.17-7.11 (m, 1H), 5.29 (s, 2H), 3.55 (s, 3H), 3.34 (q, J=7.1 Hz, 1H), 3.12 (dd, J=7.5, 1.7 Hz, 2H), 1.33 (d, J=6.9 Hz, 3H). LCMS: C₂₂H₁₉F₃N₄O₃ requires: 444.4, found: m/z 445.2 [M+H]⁺.

### Example 274: 6-((S)-3-hydroxypyrrolidine-1-carbonyl)-2-(3-((R)-1-(4-methyl-4H-1,2,4-triazol-3-yl)propan-2-yl)phenyl)-4-(trifluoromethyl)isoindolin-1-one (274)

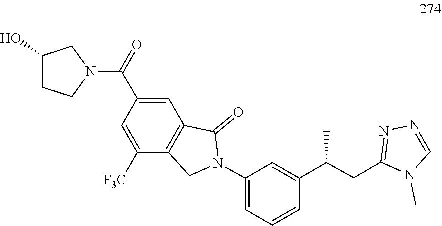 [SMILES: C[C@H](Cc1nncn1C)c1cccc(N2Cc3c(cc(C(=O)N4CC[C@H](O)C4)cc3C(F)(F)F)C2=O)c1]

Followed Example 108 to afford 6-((5)-3-hydroxypyrrolidine-1-carbonyl)-2-(3-((R)-1-(4-methyl-4H-1,2,4-triazol-3-yl)propan-2-yl)phenyl)-4-(trifluoromethyl)isoindolin-1-one as a trifluoroacetate salt. Yield: 25 mg (60%). ¹H NMR (500 MHz, DMSO-d₆) δ 8.86 (s, 1H), 8.17-8.05 (m, 2H), 7.85-7.76 (m, 2H), 7.38 (t, J=7.9 Hz, 1H), 7.17-7.11 (m, 1H), 5.25 (s, 2H), 4.30 (d, J=43.9 Hz, 1H), 3.60 (s, 3H), 3.49-3.39 (m, 1H), 3.35 (q, J=7.1 Hz, 1H), 3.18 (d, J=8.2 Hz, 2H), 2.04-1.72 (m, 2H), 1.34 (d, J=6.9 Hz, 3H). LCMS: C₂₆H₂₆F₃N₅O₃ requires: 513.5, found: m/z 514.4 [M+H]⁺.

### Example 275: 6-((2R,4S)-4-hydroxy-2-methylpyrrolidine-1-carbonyl)-2-(3-((R)-1-(4-methyl-4H-1,2,4-triazol-3-yl)propan-2-yl)phenyl)-4-(trifluoromethyl)isoindolin-1-one (275)

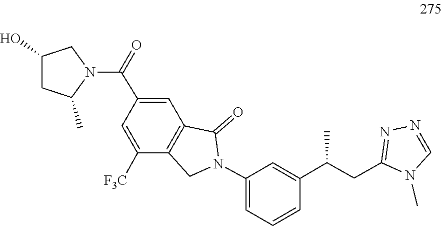 [SMILES: C[C@H](Cc1nncn1C)c1cccc(N2Cc3c(cc(C(=O)N4C[C@@H](O)C[C@H]4C)cc3C(F)(F)F)C2=O)c1]

Followed Example 108 to afford the title compound as a trifluoroacetate salt. Yield: 25 mg (60%). ¹H NMR (500 MHz, DMSO-d₆) δ 8.84 (s, 1H), 8.09 (d, J=6.2 Hz, 2H), 7.81 (t, J=3.7 Hz, 2H), 7.39 (dd, J=8.7, 7.6 Hz, 1H), 7.14 (d, J=7.8 Hz, 1H), 5.25 (s, 2H), 4.17 (dt, J=11.2, 6.3 Hz, 1H), 3.60 (s, 3H), 3.48 (dd, J=10.3, 6.0 Hz, 1H), 3.35 (q, J=7.2 Hz, 2H), 3.18 (d, J=7.0 Hz, 2H), 2.28 (dt, J=13.4, 7.0 Hz, 1H), 1.55 (dt, J=12.1, 6.2 Hz, 1H), 1.40 (d, J=6.3 Hz, 3H), 1.35 (d, J=6.9 Hz, 3H). LCMS: C₂₇H₂₈F₃N₅O₃ requires: 527.6, found: m/z 528.4 [M+H]⁺.

### Example 276: (R)-6-(3-hydroxyazetidine-1-carbonyl)-2-(3-(1-(4-methyl-4H-1,2,4-triazol-3-yl)propan-2-yl)phenyl)-4-(trifluoromethyl)isoindolin-1-one (276)

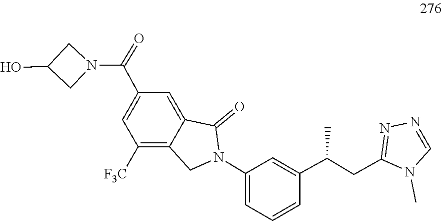 [SMILES: C[C@H](Cc1nncn1C)c1cccc(N2Cc3c(cc(C(=O)N4CC(O)C4)cc3C(F)(F)F)C2=O)c1]

Followed General procedure 1-G using azetidin-3-ol to afford the title compound. ¹H NMR (300 MHz, DMSO-d₆) δ 8.34 (s, 1H), 8.16-8.13 (m, 2H), 7.83-7.77 (m, 2H), 7.37 (t, J=7.8 Hz, 1H), 7.15 (d, J=7.8 Hz, 1H), 5.80 (br, 1H), 5.26 (s, 2H), 4.51 (s, 2H), 4.34-4.29 (m, 1H), 4.11-4.02 (m, 1H), 3.87-3.79 (m, 1H), 3.47 (s, 3H), 3.43-3.23 (m, 1H), 3.02 (d, J=7.2 Hz, 2H), 1.32 (d, J=6.9 Hz, 3H). MS (ESI) calc'd for (C₂₅H₂₄F₃N₅O₃) [M+H]⁺, 500.2; found, 500.0.

### Example 277: 4-cyclopropyl-6-((S)-3-hydroxypyrrolidine-1-carbonyl)-2-(3-((R)-1-(4-methyl-4H-1,2,4-triazol-3-yl)propan-2-yl)phenyl)isoindolin-1-one (277)

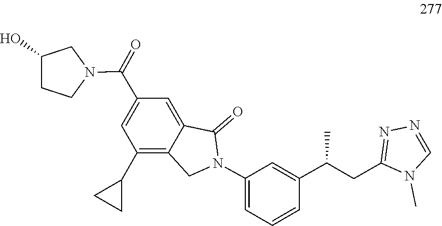 [SMILES: C[C@H](Cc1nncn1C)c1cccc(N2Cc3c(cc(C(=O)N4CC[C@H](O)C4)cc3C3CC3)C2=O)c1]

Step 1: Synthesis of methyl 5-bromo-3-iodo-2-methylbenzoate. To a stirred suspension of methyl 3-amino-5-bromo-2-methylbenzoate (49.0 g, 0.20 mol) in 6 N aqueous hydrochloric acid (115 mL) was added sodium nitrite (15.3 g, 0.2 mol, in water (80 mL)) dropwise at −5° C. over 20 min. After The mixture was stirred at −5° C. for 20 min, a solution of potassium iodide (33.2 g, 0.20 mol) in water (68 mL) and a small crystal of iodine were added slowly. The mixture was warmed to rt and heated to 90° C. for 1 h. The mixture was cooled to rt, followed by General Work-up Procedure 1. The residue was purified by flash column chromatography to afford the title compound (62.1 g, 78%) as a yellow oil. MS (ESI) calc'd for (C₉H₈BrIO₂) [M+H]⁺, 354.9; found, 355.0.

Step 2: Synthesis of methyl 5-bromo-2-(bromomethyl)-3-iodobenzoate. Using methyl 5-bromo-3-iodo-2-methylbenzoate (50.0 g, 140.86 mmol) the reaction was performed in a similar fashion to Example 247 Step 2 to afford the title compound as yellow oil (50.5 g, 83% yield). MS (ESI) calc'd for (C₉H₇Br₂IO₂) [M+H]⁺, 432.8; found, 432.8.

Step 3: Synthesis of 6-bromo-4-iodo-2,3-dihydro-1H-isoindol-1-one. To a stirred solution of methyl 5-bromo-2-(bromomethyl)-3-iodobenzoate in THF (160 mL) were added ammonia (7 M in methanol, 60 mL). The mixture was stirred at rt for 16 h. The crude product was collected by filtration and recrystallized with methanol/ethyl acetate (1/10) to afford the title compound (18.1 g, 53%) as a colorless solid: MS (ESI) calc'd for (C₈H₅BrINO) [M+H]⁺, 337.9; found, 338.0.

Step 4: Synthesis of 6-bromo-4-cyclopropyl-2,3-dihydro-1H-isoindol-1-one. To a degassed solution of 6-bromo-4-iodo-2,3-dihydro-1H-isoindol-1-one (8.5 g, 22.15 mmol) in dioxane (150 mL) and water (15 mL) were added Pd(dppf)Cl₂ (1.84 g, 2.51 mmol), potassium phosphate (10.7 g, 50.41 mmol) and cyclopropylboronic acid (2.17 g, 25.26 mmol). The solution was stirred at 65° C. for 2 days under nitrogen. The mixture was diluted with water, followed by General Work-up Procedure 1. The residue was purified by reverse phase flash chromatography to afford the title compound (2.7 g, 43%) as a brown solid. MS (ESI) calc'd for (C₁₁H₁₀BrNO) [M+H]+, 252.0; found, 252.0.

Step 5: Synthesis of methyl 7-cyclopropyl-3-oxo-2,3-dihydro-1H-isoindole-5-carboxylate. To a solution of 6-bromo-4-cyclopropyl-2,3-dihydro-1H-isoindol-1-one (3.4 g, 13.49 mmol) in methanol (150 mL) were added Pd(dppf)Cl₂ (989.0 mg, 13.93 mmol) and triethylamine (2.7 g, 26.98 mmol). The resulting system was evacuated and backfilled with CO for 3 times. The mixture was stirred for 16 h at 80° C. under CO (1 atm.). The mixture was concentrated and diluted with water, followed by General Work-up Procedure 1. The residue was purified by chromatography B to afford the title compound as a brown solid (860 mg, 28% yield). MS (ESI) calc'd for (C₁₃H₁₃NO₃) [M+H]+, 232.1; found, 232.1.

Step 6: Synthesis of (R)-methyl 7-cyclopropyl-2-(3-(1-(4-methyl-4H-1,2,4-triazol-3-yl)propan-2-yl)phenyl)-3-oxoisoindoline-5-carboxylate. To a mixture of methyl 7-cyclopropyl-3-oxoisoindoline-5-carboxylate (100 mg, 0.46 mmol) in dioxane (5 mL) were added Q (258 mg, 0.92 mmol), Pd(AcO)₂ (10 mg, 0.05 mmol), Xantphos (53 mg, 0.09 mmol) and cesium carbonate (300 mg, 0.92 mmol). The mixture was stirred at 100° C. for 16 h under nitrogen. The reaction was quenched by the addition of water, followed by General Work-up Procedure 1. The residue was purified by flash column chromatography on silica gel to afford the title compound (126 mg, 65%) as a colorless oil. MS (ESI) calc'd for (C₂₅H₂₆N₄O₃) [M+H]⁺, 431.2; found, 431.2.

Step 7: Synthesis of (R)-7-cyclopropyl-2-(3-(1-(4-methyl-4H-1,2,4-triazol-3-yl)propan-2-yl)phenyl)-3-oxoisoindoline-5-carboxylic acid. To a solution of (R)-methyl 7-cyclopropyl-2-(3-(1-(4-methyl-4H-1,2,4-triazol-3-yl)propan-2-yl)phenyl)-3-oxoisoindoline-5-carboxylate (985 mg, 2.29 mmol) in THF (10 mL) was added a solution of lithium hydroxide (109 mg, 4.58 mmol) in water (10 mL) at 0° C. The mixture was stirred at rt for 2 h. The aqueous layer was acidified with hydrochloric acid (1 N) to pH 34, followed by General Work-up Procedure 1. The residue was purified by reverse phase flash column chromatography to afford the title compound (48 mg, 5%) as a colorless solid. MS (ESI) calc'd for (C₂₄H₂₄N₄O₃) [M+H]⁺, 417.2; found, 417.2.

Step 8: Synthesis of 4-cyclopropyl-6-((S)-3-hydroxypyrrolidine-1-carbonyl)-2-(3-((R)-1-(4-methyl-4H-1,2,4-triazol-3-yl)propan-2-yl)phenyl)isoindolin-1-one. Followed Example 108 with addition of reagents at 0° C. and stirring at rt for 3 h to afford the title compound (8.5 mg, 36%) as a colorless solid. 1H NMR (300 MHz, DMSO-d₆+D₂O) δ 8.28 (s, 1H), 7.97-7.76 (m, 2H), 7.61-7.59 (m, 1H), 7.36 (t, J=8.1 Hz, 1H), 7.25 (s, 1H), 7.09 (d, J=7.5 Hz, 1H), 5.12 (s, 2H), 4.33-4.23 (m, 1H), 3.58-3.50 (m, 2H), 3.44 (s, 3H), 3.37-3.27 (m, 2H), 3.15-3.13 (m, 1H), 3.01 (d, J=7.2 Hz, 2H), 2.13-2.07 (m, 1H), 2.02-1.71 (m, 2H), 1.31 (d, J=6.9 Hz, 3H), 1.16-1.01 (m, 2H), 0.95-0.79 (m, 2H). MS (ESI) calc'd for (C₂₈H₃₁N₅O₃) [M+H]⁺, 486.2; found, 486.2.

### Example 278: (R)-4-cyclopropyl-6-(3-hydroxyazetidine-1-carbonyl)-2-(3-(1-(4-methyl-4H-1,2,4-triazol-3-yl)propan-2-yl)phenyl)isoindolin-1-one (278)

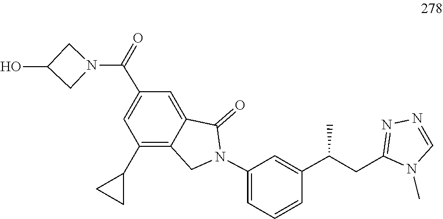 [SMILES: C[C@H](Cc1nncn1C)c1cccc(N2Cc3c(cc(C(=O)N4CC(O)C4)cc3C3CC3)C2=O)c1]

Followed Example 277, step 8 to afford the title compound (5.0 mg, 22%). MS (ESI) calc'd for (C₂₇H₂₉N₅O₃) [M+H]⁺, 472.2; found, 472.2. ¹H NMR (300 MHz, DMSO-d₆+D₂O) δ 8.53 (s, 1H), 7.90-7.75 (m, 2H), 7.70-7.68 (m, 1H), 7.53-7.26 (m, 2H), 7.12-7.09 (m, 1H), 5.13 (s, 2H), 4.52-4.47 (m, 2H), 4.29-4.26 (m, 1H), 4.04-4.01 (m, 1H), 3.84-3.80 (m, 1H), 3.51 (s, 3H), 3.39-3.27 (m, 1H), 3.09 (d, J=7.5 Hz, 2H), 2.16-2.04 (m, 1H), 1.33 (d, J=6.9 Hz, 3H), 1.11-1.07 (m, 2H), 0.90-0.85 (m, 2H).

### Example 279: (R)-2-(3-(1-(4-methyl-4H-1,2,4-triazol-3-yl)propan-2-yl)phenyl)-6-(1H-pyrazol-5-yl)-4-(trifluoromethyl)isoindolin-1-one (279)

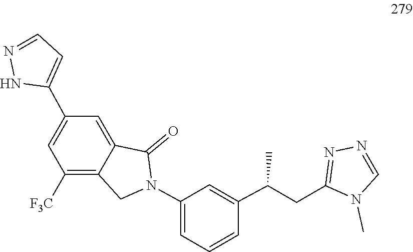 [SMILES: C[C@H](Cc1nncn1C)c1cccc(N2Cc3c(cc(-c4ccn[nH]4)cc3C(F)(F)F)C2=O)c1]

Step 1: Synthesis of 6-(1-(tetrahydro-2H-pyran-2-yl)-1H-pyrazol-5-yl)-4-(trifluoromethyl)isoindolin-1-one. To a degassed solution of 6-bromo-4-(trifluoromethyl)isoindolin-1-one (1.0 g, 3.57 mmol) in dioxane (20 mL) and water (2 mL) were added 1-(oxan-2-yl)-5-(4,4,5,5-tetramethyl-1,3,2-dioxaborolan-2-yl)-1H-pyrazole (1.5 g, 5.39 mmol), sodium carbonate (760 mg, 7.17 mmol) and Pd(dppf)Cl₂ (30 mg, 0.04 mmol). The mixture was stirred at 100° C. for 16 h. The mixture was diluted with water and extracted with EtOAc. The organic layers were dried, filtered and concentrated. The residue was purified by flash column chromatography with 0-10% methanol in DCM to afford the title compound (1.0 g, 80%) as a yellow solid. MS (ESI) calc'd for C₁₇H₁₆F₃N₃O₂, 352.1; found, 268.0 [M-THP+H]⁺.

Step 2: Synthesis of 2-(3-((R)-1-(4-methyl-4H-1,2,4-triazol-3-yl)propan-2-yl)phenyl)-6-(1-(tetrahydro-2H-pyran-2-yl)-1H-pyrazol-5-yl)-4-(trifluoromethyl)isoindolin-1-one. Using 6-[1-(oxan-2-yl)-1H-pyrazol-5-yl]-4-(trifluoromethyl)isoindolin-1-one (900 mg, 2.56 mmol) and Q (858 mg, 3.06 mmol) the reaction was performed in a similar fashion to Example 277 Step 6 to afford the title compound (380 mg, 27%) as a yellow oil. MS (ESI) calc'd for (C₂₉H₂₉F₃N₆O₂) [M+H]⁺, 551.2; found, 551.1.

Step 3: Synthesis of 2-[3-[(2R)-1-(4-methyl-4H-1,2,4-triazol-3-yl)propan-2-yl]phenyl]-6-(1H-pyrazol-5-yl)-4-(trifluoromethyl)isoindolin-1-one. A mixture of 2-[3-[(2R)-1-(4-methyl-4H-1,2,4-triazol-3-yl)propan-2-yl]phenyl]-6-[1-(oxan-2-yl)-1H-pyrazol-5-yl]-4-(trifluoromethyl)isoindolin-1-one (360 mg, 0.65 mmol) and hydrochloric acid (4 M in dioxane) (5 mL) was stirred at rt for 2 h. The mixture was concentrated. The crude product was purified by Prep-HPLC to afford the title compound (33.9 mg, 11%) as a colorless solid. 1H NMR (300 MHz, DMSO-d₆) δ 13.18 (br, 1H), 8.44-8.42 (m, 3H), 7.85-7.81 (m, 3H), 7.41-7.35 (m, 1H), 7.14-7.08 (m, 1H), 7.07 (d, J=2.4 Hz, 1H), 5.21 (s, 2H), 3.48 (s, 3H), 3.32-3.25 (m, 1H), 3.06 (d, J=7.5 Hz, 2H), 1.33 (d, J=6.8 Hz, 3H). MS (ESI) calc'd for (C₂₄H₂₁F₃N₆O) [M+H]⁺, 467.2; found, 467.1.

### Example 280: 2-[4-fluoro-3-[(1R)-1-methyl-2-(4-methyl-1,2,4-triazol-3-yl)ethyl]phenyl]-4-(trifluoromethyl)isoindolin-1-one (280)

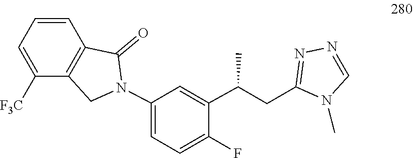 [SMILES: C[C@H](Cc1nncn1C)c1cc(N2Cc3c(cccc3C(F)(F)F)C2=O)ccc1F]

Step 1: Ethyl (E)-3-(5-bromo-2-fluoro-phenyl)but-2-enoate. To a stirring solution of sodium hydride (1.97 g, 49.2 mmol, 11 eq.) in tetrahydrofuran (40 mL) at 0° C., was added triethyl phosphonoacetate (1.52 mL, 7.37 mmol, 1.6 eq.). The reaction was allowed to proceed for 30 minutes at room temperature and then a solution of l-(5-bromo-2-fluoro-phenyl)ethanone (1 g, 4.61 mmol, 1 eq.) in tetrahydrofuran (20 mL) was added. The reaction was allowed to proceed for 12 h at room temperature. The solution was concentrated then purified by flash column chromatography to give ethyl (E)-3-(5-bromo-2-fluoro-phenyl)but-2-enoate (1.2 g, 4.18 mmol, 91% yield) as a clear oil.

Step 2: (E)-3-[2-fluoro-3-[1-oxo-4-(trifluoromethyl)isoindolin-2-yl]phenyl]but-2-enoate. Using ethyl (E)-3-(5-bromo-2-fluoro-phenyl)but-2-enoate (1.2 g, 4.18 mmol, 1 eq.), and 4-(trifluoromethyl)isoindolin-1-one (1.7 g, 8.36 mmol, 2 eq.) the reaction was performed in a similar fashion to Example 277 Step 6 to give ethyl (E)-3-[2-fluoro-5-[1-oxo-4-(trifluoromethyl)isoindolin-2-yl]phenyl]but-2-enoate (700 mg, 1.72 mmol, 41% yield) as a white solid.

Step 3: Ethyl (E)-3-[2-fluoro-5-[1-oxo-4-(trifluoromethyl)isoindolin-2-yl]phenyl]but-2-enoate. To a solution of ethyl (E)-3-[2-fluoro-3-[1-oxo-4-(trifluoromethyl)isoindolin-2-yl]phenyl]but-2-enoate (700 mg, 1.72 mmol, 1 eq.) in ethanol (11 mL) under nitrogen was added 10% palladium on carbon (50 mg). The reaction was put under a hydrogen balloon and stirred for 3 h. The suspension was then filtered over celite and concentrated. The crude product was used on the next step.

Step 4: 3-[2-Fluoro-5-[1-oxo-4-(trifluoromethyl)isoindolin-2-yl]phenyl]butanoic acid. To a solution of ethyl 3-[2-fluoro-5-[1-oxo-4-(trifluoromethyl)isoindolin-2-yl]phenyl]butanoate (700 mg, 1.71 mmol, 1.0 eq.) in tetrahydrofuran/ethanol/water (2:2:2, 10 mL) was added lithium hydroxide (0.72 g, 17.1 mmol, 10 eq.). The reaction was diluted with 1 M hydrochloric acid and the aqueous layer was extracted with ethyl acetate. The combined organic layers were washed were dried over sodium sulfate, filtered, and concentrated in vacuo. The crude product was used on the next step.

Step 5: 1-[3-[2-Fluoro-5-[1-oxo-4-(trifluoromethyl)isoindolin-2-yl]phenyl]butanoylamino]-3-methyl-thiourea. To a stirring solution of 3-[2-fluoro-5-[1-oxo-4-(trifluoromethyl)isoindolin-2-yl]phenyl]butanoic acid (200 mg, 0.52 mmol, 1 eq.), EDC (121 mg, 0.63 mmol, 1.2 eq.), methylaminothiourea (105 mg, 0.79 mmol, 1.5 eq.), and 1-hydroxybenzotriazole hydrate (96 mg, 0.63 mmol, 1.2 eq) in DMF (4 mL) was added 4-methylmorpholine (0.29 mL, 2.62 mmol, 5 eq.). The reaction mixture was stirred for about 24 h at room temperature. The reaction was diluted with water and extracted with ethyl acetate. The combined organic layers were washed were dried over sodium sulfate, filtered, and concentrated under reduced pressure. The crude was used in the next step.

Step 6: 2-[4-Fluoro-3-[1-methyl-2-(4-methyl-5-thioxo-1H-1,2,4-triazol-3-yl)ethyl]phenyl]-4-(trifluoromethyl)-isoindolin-1-one. To a stirring solution of l-[3-[2-fluoro-5-[1-oxo-4-(trifluoromethyl)isoindolin-2-yl]phenyl]butanoylamino]-3-methyl-thiourea in methanol (3.8 mL) was added sodium methoxide (36 mg, 0.64 mmol, 1 eq.). The mixture was stirred at 60° C. for about 2 h and then cooled to room temperature. The reaction was diluted with 1 M hydrochloric acid and extracted with ethyl acetate. The combined organic layers were washed were dried over sodium sulfate, filtered, and concentrated under reduced pressure. The crude was used in the next step.

Step 7: 2-[4-Fluoro-3-[1-methyl-2-(4-methyl-1,2,4-triazol-3-yl)ethyl]phenyl]-4-(trifluoromethyl)isoindolin-1-one. To a stirring solution of 2-[4-fluoro-3-[1-methyl-2-(4-methyl-5-thioxo-1H-1,2,4-triazol-3-yl)ethyl]phenyl]-4-(trifluoromethyl)^{_}, isoindolin-1-one in tetrahydrofuran (6 mL) and nitric acid (1.2 mL, 6.14 mmol, 10 eq.) was added sodium nitrite (423 mg, 6.13 mmol, 10 eq.). The mixture was stirred at 0° C. for about 1 h. The reaction was diluted with water and extracted with ethyl acetate. The combined organic layers were washed were dried over sodium sulfate, filtered, and concentrated under reduced pressure. The reaction mixture was purified by HPLC to afford 2-[4-fluoro-3-[1-methyl-2-(4-methyl-1,2,4-triazol-3-yl)ethyl]phenyl]-4-(trifluoromethyl)isoindolin-1-one (30 mg, 0.07 mmol, 12% yield).

Step 8: 2-[4-Fluoro-3-[(1R)-1-methyl-2-(4-methyl-1,2,4-triazol-3-yl)ethyl]phenyl]-4-(trifluoromethyl)isoindolin-1-one. The racemic compound was separated by chiral-SFC to afford the title compound: 1H NMR (500 MHz, methanol-d4) δ 8.30 (s, 1H), 8.12-8.08 (m, 1H), 8.00-7.94 (m, 1H), 7.86 (dd, J=6.5, 2.8 Hz, 1H), 7.77 (t, J=7.7 Hz, 1H), 7.71-7.64 (m, 1H), 7.13 (dd, J=10.0, 9.0 Hz, 1H), 5.17 (s, 2H), 3.67 (dt, J=15.1, 7.5 Hz, 1H), 3.60 (s, 3H), 3.17 (d, J=7.6 Hz, 2H), 1.43 (d, J=7.0 Hz, 3H). LCMS: C₂₁H₁₈F₄N₄O requires: 418.4, found: m/z=419.4 [M+H]⁺.

### Example 281: 2-[3-Fluoro-5-[(1R)-1-methyl-2-(4-methyl-1,2,4-triazol-3-yl)ethyl]phenyl]-4-(trifluoromethyl)isoindolin-1-one (281a) and 2-[3-Fluoro-5-[(1S)-1-methyl-2-(4-methyl-1,2,4-triazol-3-yl)ethyl]phenyl]-4-(trifluoromethyl)isoindolin-1-one (281b)

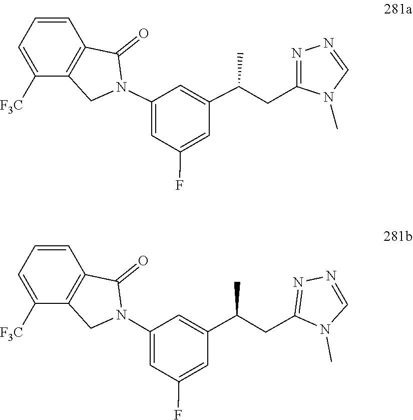 [SMILES: C[C@H](Cc1nncn1C)c1cc(F)cc(N2Cc3c(cccc3C(F)(F)F)C2=O)c1 ; C[C@@H](Cc1nncn1C)c1cc(F)cc(N2Cc3c(cccc3C(F)(F)F)C2=O)c1]

Step 1: Ethyl (E)-3-(5-bromo-3-fluoro-phenyl)but-2-enoate. Followed Example 280, step 1 to the title compound (4.01 g, 14.0 mmol, 87% yield) as a clear oil.

Step 2: Ethyl (E)-3-(3-fluoro-5-(1-oxo-4-(trifluoromethyl)isoindolin-2-yl)phenyl)but-2-enoate. Followed Example 168, step 1 to give the title compound (5.6 g, 13.75 mmol, 94% yield) as a colorless solid.

Step 3: Ethyl 3-(3-fluoro-5-(1-oxo-4-(trifluoromethyl)isoindolin-2-yl)phenyl)butanoate. To a solution of ethyl (E)-3-[3-fluoro-5-[1-oxo-4-(trifluoromethyl)isoindolin-2-yl]phenyl]but-2-enoate (5.6 g, 13.75 mmol, 1 eq.) in ethanol (70 mL) and DCM (70 mL) under nitrogen was added 10% palladium on carbon (250 mg). The reaction was put under a hydrogen balloon and stirred for 3 h. The suspension is then filtered over celite and concentrated. The crude product was used.

Step 4: 3-[3-Fluoro-5-[1-oxo-4-(trifluoromethyl)isoindolin-2-yl]phenyl]butanehydrazide. To a stirring solution of ethyl 3-[3-fluoro-5-[1-oxo-4-(trifluoromethyl) isoindolin-2-yl]phenyl]butanoate (2.0 g, 4.89 mmol, 1 eq.) in ethanol (48 mL) was added hydrazine (1.23 mL, 9.77 mmol, 2 eq.). The mixture was stirred at 80° C. for about 12 h, cooled to rt, and then concentrated. The reaction was diluted with water and extracted with EtOAc. The combined organic layers were washed were dried, filtered, and concentrated. The crude was used without purification

Step 5: 2-[3-Fluoro-5-[1-methyl-2-(4-methyl-5-sulfanyl-1,2,4-triazol-3-yl)ethyl]phenyl]-4-(trifluoromethyl)isoindolin-1-one. To a stirring solution of 3-[3-fluoro-5-[l-oxo-4-(trifluoromethyl) isoindolin-2-yl]phenyl]butanehydrazide in THF (60 mL) was added methylimino (thioxo)methane (0.4 mL, 5.82 mmol, 1 eq.). The mixture was stirred at rt for about 16 h. Added 60 mL of 2 M potassium hydroxide (60 mL) and stirred the solution for 2 h. The reaction was diluted with 1 M hydrochloric acid and the aqueous layer was extracted with EtOAc. The combined organic layers were washed were dried, filtered, and concentrated under reduced pressure. The crude was used without purification.

Step 6: 2-[3-Fluoro-5-[1-methyl-2-(4-methyl-1,2,4-triazol-3-yl)ethyl]phenyl]-4-(trifluoro methyl)isoindolin-1-one. To a stirring solution of 2-[3-fluoro-5-[1-methyl-2-(4-methyl-5-sulfanyl-1,2,4-triazol-3-yl)ethyl]phenyl]-4-(trifluoromethyl)isoindolin-1-one in DCM (20 mL) and acetic acid (20 mL) was added hydrogen peroxide (0.7 mL). The mixture was stirred at rt for about 14 h. The mixture was concentrated then purified by flash column chromatography to give the title compound (0.65 g, 1.55 mmol, 32% yield) as a colorless solid.

Step 7: 2-[3-fluoro-5-[(1R)-1-methyl-2-(4-methyl-1,2,4-triazol-3-yl)ethyl]phenyl]-4-(trifluoromethyl)isoindolin-1-one. The racemic product was separated by chiral-SFC to afford 111 mg of the title compound. 1H NMR (500 MHz, methanol-d4) δ 8.28 (s, 1H), 8.07 (d, J=7.6 Hz, 1H), 7.98-7.94 (m, 1H), 7.75 (t, J=7.7 Hz, 1H), 7.70 (dt, J=11.0, 2.2 Hz, 1H), 7.47 (t, J=1.6 Hz, 1H), 6.88 (ddd, J=9.4, 2.4, 1.2 Hz, 1H), 5.13 (d, J=6.3 Hz, 2H), 3.54 (s, 3H), 3.39 (q, J=7.2 Hz, 1H), 3.12 (dd, J=7.6, 4.5 Hz, 2H), 1.41 (d, J=6.9 Hz, 3H). LCMS: C₂₁H₁₈F₄N₄O requires: 418.3, found: m/z=419.4 [M+H]⁺

2-[3-fluoro-5-[(1S)-1-methyl-2-(4-methyl-1,2,4-triazol-3-yl)ethyl]phenyl]-4-(trifluoromethyl) isoindolin-1-one. The racemic product was separated by chiral-SFC to afford 96 mg the title compound. ¹H NMR (500 MHz, methanol-d4) δ 8.28 (s, 1H), 8.07 (d, J=7.6 Hz, 1H), 7.98-7.94 (m, 1H), 7.75 (t, J=7.7 Hz, 1H), 7.70 (dt, J=11.0, 2.2 Hz, 1H), 7.47 (t, J=1.6 Hz, 1H), 6.88 (ddd, J=9.4, 2.4, 1.2 Hz, 1H), 5.13 (d, J=6.3 Hz, 2H), 3.54 (s, 3H), 3.39 (q, J=7.2 Hz, 1H), 3.12 (dd, J=7.6, 4.5 Hz, 2H), 1.41 (d, J=6.9 Hz, 3H). LCMS: C₂₁H₁₈F₄N₄O requires: 418.3, found: m/z=419.4 [M+H]⁺

### Example 282: 2-(3-((1S,2R)-2-(4-methyl-4H-1,2,4-triazol-3-yl)cyclopropyl)phenyl)-4-(trifluoromethyl)isoindolin-1-one (282)

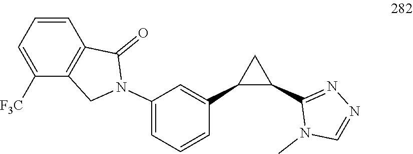 [SMILES: Cn1cnnc1[C@@H]1C[C@@H]1c1cccc(N2Cc3c(cccc3C(F)(F)F)C2=O)c1]

Using 3-((1S,2R)-2-(4-methyl-4H-1,2,4-triazol-3-yl)cyclopropyl)benzeneamine P1(70.0 mg, 0.24 mmol) and methyl 2-(bromomethyl)-3-(trifluoromethyl)benzoate (50.4 mg, 0.24 mmol) to the reaction was performed in a similar fashion to Example 256 to afford the title compound (50.7 mg, 54%) as a light yellow solid. MS (ESI) calculated for (C₂₁H₁₇F₃N₄O) [M+H]⁺, 399.1; found, 399.0. ¹H NMR (400 MHz, DMSO-d₆) δ 8.15 (s, 1H), 8.08-8.02 (m, 2H), 7.81-7.77 (m, 1H), 7.72-7.70 (m, 1H), 7.57 (s, 1H), 7.18-7.14 (m, 1H), 6.74 (d, J=8.0 Hz, 1H), 5.13-5.02 (m, 2H), 3.42 (s, 3H), 2.69-2.63 (m, 1H), 2.55-2.53 (m, 1H), 1.92-1.88 (m, 1H), 1.62-1.59 (m, 1H).

### Example 283: cis-2-(3-(2-(4-Methyl-4H-1,2,4-triazol-3-yl)cyclobutyl)phenyl)-4-(trifluoromethyl)isoindolin-1-one (283)

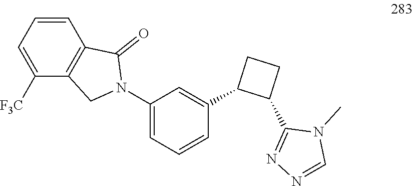 [SMILES: Cn1cnnc1[C@H]1CC[C@H]1c1cccc(N2Cc3c(cccc3C(F)(F)F)C2=O)c1]

Step 1: Synthesis of 1,1-dimethyl 2-(3-nitrophenyl)cyclobutane-1,1-dicarboxylate. To a solution of 1,3-dimethyl propanedioate (14.2 g, 107.5 mmol) in DMF (500 mL) was added sodium hydride (4.4 g, 110.01 mmol, 60%) at 10° C. under nitrogen. The mixture was stirred at 10° C. for 30 min. To the above solution was added a solution of l-(1,3-dibromopropyl)-3-nitrobenzene (Nair, V. et. al., Res. Chem. Intermediates, 2003, 29, 227-231) (29.8 g, 92.27 mmol) in DMF (100 mL). The mixture was stirred at 50° C. for 1 h. Another batch of sodium hydride (5.1 g, 127.51 mmol, 60%) was added to the mixture at 50° C. and stirred at 50° C. for another 1 h. The mixture was diluted with EtOAc, washed with brine, dried, and filtered. The filtrate was evaporated in vacuo. The residue was purified by flash column chromatography using 5-30% EtOAc in petroleum ether to afford the title compound (25.4 g, 56%) as a light yellow solid: MS (ESI) calculated for (C₁₄H₁₅NO₆) [M+H]⁺, 294.1, found, 293.9.

Step 2: Synthesis of methyl 2-(3-nitrophenyl)cyclobutane-1-carboxylate. A mixture of 1,1-dimethyl 2-(3-nitrophenyl)cyclobutane-1,1-dicarboxylate (25.4 g, 86.61 mmol) and lithium chloride (9.7 g, 228.81 mmol) in DMSO (400 mL) was heated to 140° C. for 7 h. The mixture was cooled to rt, and then diluted with water, extracted with EtOAc. The combined organic layers were washed with brine, dried, and filtered. The filtrate was evaporated in vacuo. The residue was purified by flash column chromatography with 20% EtOAc in petroleum ether to afford the title compound (13.5 g, 66%) as a yellow oil.

Step 3: Synthesis of 2-(3-nitrophenyl)cyclobutane-1-carbohydrazide. A mixture of methyl 2-(3-nitrophenyl)cyclobutane-1-carboxylate (13.5 g, 57.39 mmol) and hydrazine (100 mL, 80%) in ethanol (100 mL) was heated to 80° C. for 16 h. The mixture was evaporated in vacuo to afford the title compound (15.3 g, crude) as a yellow oil, which was used without purification. MS (ESI) calculated for (C₁₁H₁₃N₃O₃) [M+H]⁺, 236.1, found, 235.9.

Step 4: Synthesis of (E)-N,N-dimethyl-N′-(2-(3-nitrophenyl)cyclobutanecarbonyl)formohydrazonamide. To a solution of 2-(3-nitrophenyl)cyclobutanecarbohydrazide (15.3 g, crude) in DCM (150 mL) was added dimethylformamide dimethyl acetal (30 mL, 224.00 mmol). The mixture was stirred at rt for 16 h. The mixture was evaporated in vacuo to afford the title compound (18.0 g, crude) as a brown oil. MS (ESI) calculated for (C₁₄H₁₈N₄O₃) [M+H]⁺, 291.1; found, 290.9.

Step 5: Synthesis of 4-methyl-3-(2-(3-nitrophenyl)cyclobutyl)-4H-1,2,4-triazole. To a solution of (E)-N,N-dimethyl-N′-(2-(3-nitrophenyl)cyclobutanecarbonyl)formohydrazonamide (1.0 g, 3.44 mmol) in acetic acid (15 mL) was added methylamine (15 mL, 2 M in THF). The mixture was stirred at 90° C. for 3 h. The solvent was evaporated under reduced pressure. The residue was diluted with water, adjusted to pH 8-9 with aqueous sodium bicarbonate and extracted with EtOAc. The combined organic layers were was washed with brine, dried, and filtered. The filtrate was evaporated in vacuo. The residue was purified by flash column chromatography with 0-10% methanol in DCM to the title compound (275 mg, 31%) as a yellow solid. MS (ESI) calculated for (C₁₃H₁₄N₄O₂) [M+H]⁺, 259.1; found, 259.1.

Step 6: Synthesis of 3-(2-(4-methyl-4H-1,2,4-triazol-3-yl)cyclobutyl)aniline. To a solution of 4-methyl-3-(2-(3-nitrophenyl)cyclobutyl)-4H-1,2,4-triazole (600.0 mg, 2.32 mmol) in methanol (30 mL) was added palladium on carbon (200 mg). The mixture was stirred at rt for 16 h under hydrogen. The mixture was filtered. The filtrate was concentrated to afford the title compound (500.0 mg, crude) as a yellow oil. MS (ESI) calculated for (C₁₃H₁₆N₄) [M+H]⁺, 229.1; found, 229.0.

Step 7: trans-2-(3-(2-(4-methyl-4H-1,2,4-triazol-3-yl)cyclobutyl)phenyl)-4-(trifluoromethyl)isoindolin-1-one and cis-2-(3-(2-(4-methyl-4H-1,2,4-triazol-3-yl)cyclobutyl)phenyl)-4-(trifluoromethyl)isoindolin-1-one. To a solution of 3-(2-(4-methyl-4H-1,2,4-triazol-3-yl)cyclobutyl)aniline (400.0 mg, 1.75 mmol) in ethanol (10 mL) were added methyl 2-(bromomethyl)-3-(trifluoromethyl)benzoate (521.0 mg, 1.75 mmol) and triethylamine (532.0 mg, 5.25 mmol). The mixture was stirred at 80° C. for 16 h before concentration under vacuum. The residue was purified by Prep-HPLC to afford:

trans-2-(3-(2-(4-methyl-4H-1,2,4-triazol-3-yl)cyclobutyl)phenyl)-4-(trifluoromethyl)isoindolin-1-one (211 mg, 29%, colorless solid): ¹H NMR (300 MHz, Chloroform-d) δ 8.35 (s, 1H), 8.11-8.03 (m, 2H), 7.91 (s, 1H), 7.83-7.78 (m, 2H), 7.43-7.38 (m, 1H), 7.23 (d, J=7.8 Hz, 1H), 5.24 (s, 2H), 3.99-3.90 (m, 1H), 3.82-3.73 (m, 1H), 3.45 (s, 3H), 2.38-2.21 (m, 4H). MS (ESI) calculated for (C₂₂H₁₉F₃N₄O) [M+H]⁺, 413.2; found, 413.0. cis-2-(3-(2-(4-methyl-4H-1,2,4-triazol-3-yl)cyclobutyl)phenyl)-4-(trifluoromethyl)isoindolin-1-one (10.0 mg, 1%, colorless solid): 1H NMR (300 MHz, Chloroform-d) δ 8.15 (s, 1H), 8.12-8.03 (m, 2H), 7.82-7.78 (m, 2H), 7.46 (d, J=1.5 Hz, 1H), 7.23-7.18 (m, 1H), 6.80 (d, J=7.8 Hz, 1H), 5.10 (s, 2H), 4.15-4.03 (m, 2H), 3.05 (s, 3H), 2.80-2.72 (m, 1H), 2.66-2.57 (m, 1H), 2.47-2.32 (m, 2H). MS (ESI) calculated for (C₂₂H₁₉F₃N₄O) [M+H]⁺, 413.2; found, 413.0.

### 2-(3-((1R,2S)-2-(4-methyl-4H-1,2,4-triazol-3-yl)cyclobutyl)phenyl)-4-(trifluoromethyl) isoindolin-1-one (283a) and 2-(3-((1S,2R)-2-(4-methyl-4H-1,2,4-triazol-3-yl)cyclobutyl)phenyl)-4-(trifluoromethyl)isoindolin-1-one (283b)

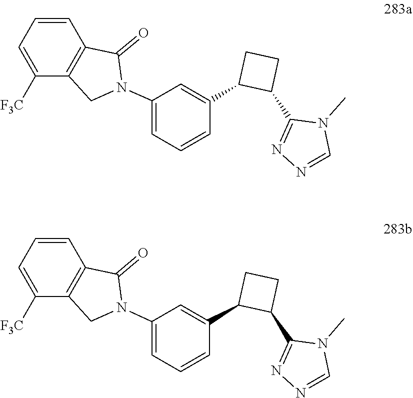 [SMILES: Cn1cnnc1[C@H]1CC[C@H]1c1cccc(N2Cc3c(cccc3C(F)(F)F)C2=O)c1 ; Cn1cnnc1[C@@H]1CC[C@@H]1c1cccc(N2Cc3c(cccc3C(F)(F)F)C2=O)c1]

Racemic cis-2-(3-(2-(4-methyl-4H-1,2,4-triazol-3-yl)cyclobutyl)phenyl)-4-(trifluoromethyl)isoindolin-1-one (150.0 mg, 0.36 mmol) was separated by chiral-SFC to afford:

2-(3-((1R,2S)-2-(4-methyl-4H-1,2,4-triazol-3-yl)cyclobutyl)phenyl)-4-(trifluoromethyl)isoindolin-1-one (shorter retention time, 24.9 mg, 33%): MS (ESI) calculated for (C₂₂H₁₉F₃N₄O) [M+H]⁺, 413.2; found, 413.0. ¹H NMR (400 MHz, DMSO-d₆) δ 8.13 (s, 1H), 8.08-8.03 (m, 2H), 7.81-7.78 (m, 2H), 7.45 (s, 1H) 7.22-7.18 (m, 1H), 6.80 (d, J=8.0 Hz, 1H), 5.12-5.01 (m, 2H), 4.11-4.03 (m, 2H), 3.05 (s, 3H), 2.77-2.72 (m, 1H), 2.62-2.51 (m, 1H), 2.50-2.4 (m, 1H), 2.40-2.36 (m, 1H).

2-(3-((1S,2R)-2-(4-methyl-4H-1,2,4-triazol-3-yl)cyclobutyl)phenyl)-4-(trifluoromethyl)isoindolin-1-one (longer retention time, 37.6 mg, 50% yield, colorless solid): MS (ESI) calculated for C₂₂H₁₉F₃N₄O) [M+H]⁺, 413.2; found, 413.0. ¹H NMR (400 MHz, DMSO-d₆) δ 8.13 (s, 1H), 8.06-8.03 (m, 2H), 7.81-7.77 (m, 2H), 7.45 (s, 1H) 7.22-7.18 (m, 1H), 6.80 (d, J=7.6 Hz, 1H), 5.12-5.06 (m, 2H), 4.11-4.05 (m, 2H), 3.05 (s, 3H), 2.77-2.72 (m, 1H), 2.62-2.51 (m, 1H), 2.50-2.44 (m, 1H), 2.40-2.35 (m, 1H).

### Example 284: N-(3-(2-methyl-1-(4-methyl-4H-1,2,4-triazol-3-yl)propan-2-yl)phenyl)-6-(trifluoromethyl)picolinamide (284)

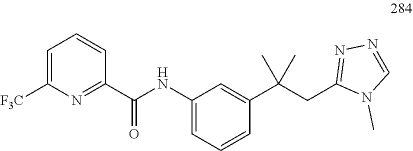 [SMILES: Cn1cnnc1CC(C)(C)c1cccc(NC(=O)c2cccc(C(F)(F)F)n2)c1]

Followed General procedure 6 to afford the title compound (49.0 mg, 28%) as a light yellow solid. ¹H NMR (300 MHz, DMSO-d₆) δ 10.34 (s, 1H), 8.47 (d, J=3.3 Hz, 1H), 8.41-8.36 (m, 2H), 8.20-8.14 (m, 1H), 7.80-7.74 (m, 2H), 7.33-7.28 (m, 1H), 7.10 (d, J=7.8 Hz, 1H), 3.19 (d, J=6.9 Hz, 3H), 3.06 (s, 2H), 1.44 (s, 6H). MS (ESI) calculated for (C₂₀H₂₀F₃N₅O) [M+H]⁺, 404.2; found, 403.9.

### Example 285: N-(3-(2-methyl-1-(4-methyl-4H-1,2,4-triazol-3-yl)propan-2-yl)phenyl)-2-(trifluoromethyl)pyrimidine-4-carboxamide (285)

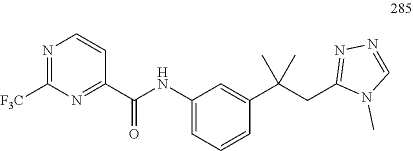 [SMILES: Cn1cnnc1CC(C)(C)c1cccc(NC(=O)c2ccnc(C(F)(F)F)n2)c1]

Followed General procedure 6 to afford the title compound (105 mg, 60%) as a light yellow solid. ¹H NMR (300 MHz, DMSO-d₆) δ 10.61 (s, 1H), 9.36 (d, J=5.1 Hz, 1H), 8.43 (s, 1H), 8.37 (d, J=5.1 Hz, 1H), 7.79-7.77 (m, 2H), 7.34-7.29 (m, 1H), 7.13 (d, J=7.5 Hz, 1H), 3.19 (s, 3H), 3.04 (d, J=5.1 Hz, 2H), 1.44 (s, 6H). MS (ESI) calculated for (C₁₉H₁₉F₃N₆O) [M+H]⁺, 405.2; found, 404.9.

### Example 286: 6-cyclopropyl-N-(3-(1,1-difluoro-1-(4-methyl-4H-1,2,4-triazol-3-yl)propan-2-yl)phenyl)-5-methylpicolinamide (286)

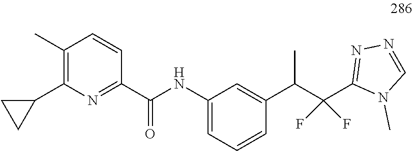 [SMILES: Cc1ccc(C(=O)Nc2cccc(C(C)C(F)(F)c3nncn3C)c2)nc1C1CC1]

Followed General procedure 6 with addition of reagents at 0° C. to afford the title compound (13.9 mg, 28%) as a colorless solid. 1H NMR (300 MHz, DMSO-d₆) δ 10.04 (s, 1H), 8.59 (s, 1H), 7.82-7.72 (m, 4H), 7.33 (t, J=7.8 Hz, 1H), 7.07 (d, J=7.8 Hz, 1H), 4.08-3.95 (m, 1H), 3.60 (s, 3H), 2.50 (s, 3H), 2.31-2.22 (m, 1H), 1.51 (d, J=7.2 Hz, 3H), 1.27-1.22 (m, 2H), 1.06-1.00 (m, 2H). ¹⁹F NMR (282 MHz, DMSO-d₆) δ-95.50, −96.45, −99.85, −100.80. MS (ESI) calc'd for (C₂₂H₂₃F₂N₅O) [M+H]⁺, 412.2; found, 412.2.

### Example 287: 6-cyclopropyl-N-(3-(1,1-difluoro-1-(4-methyl-4H-1,2,4-triazol-3-yl)propan-2-yl)phenyl)-5-(hydroxymethyl)picolinamide (287)

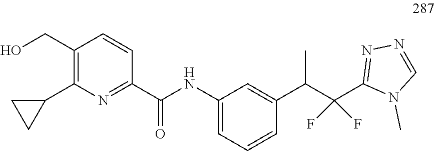 [SMILES: CC(c1cccc(NC(=O)c2ccc(CO)c(C3CC3)n2)c1)C(F)(F)c1nncn1C]

Followed General procedure 6 with addition of reagents at 0° C. to afford the title compound (9.3 mg, 18%) as a colorless solid. ¹H NMR (300 MHz, DMSO-d₆) δ 10.06 (s, 1H), 8.59 (s, 1H), 7.96-7.88 (m, 2H), 7.82 (s, 1H), 7.76-7.73 (m, 1H), 7.34 (t, J=7.8 Hz, 1H), 7.07 (d, J=7.8 Hz, 1H), 5.49 (t, J=5.4 Hz, 1H), 4.79 (d, J=5.4 Hz, 2H), 4.08-3.95 (m, 1H), 3.60 (s, 3H), 2.27-2.18 (m, 1H), 1.51 (d, J=7.2 Hz, 3H), 1.29-1.25 (m, 2H), 1.04-0.99 (m, 2H). ¹⁹F NMR (282 MHz, DMSO-d₆) δ −95.49, −96.44, −99.85, −100.81. MS (ESI) calc'd for (C₂₂H₂₃F₂N₅O₂) [M+H]⁺, 428.2; found, 428.2.

### Example 288: Trans-6-cyclopropyl-N-(3-(1-hydroxy-1-(4-methyl-4H-1,2,4-triazol-3-yl)propan-2-yl)phenyl)-5-methylpicolinamide (288)

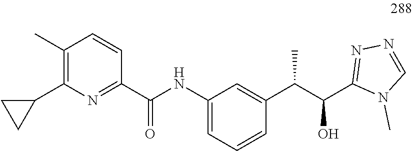 [SMILES: Cc1ccc(C(=O)Nc2cccc([C@H](C)[C@H](O)c3nncn3C)c2)nc1C1CC1]

Compound 288 (36.6 mg, 54%) was obtained according to procedures disclosed herein as a colorless solid. ¹H NMR (300 MHz, DMSO-d₆) δ 10.00 (s, 1H), 8.37 (s, 1H), 7.83-7.70 (m, 4H), 7.31 (t, J=7.8 Hz, 1H), 7.07 (d, J=7.5 Hz, 1H), 5.65 (d, J=5.7 Hz, 1H), 4.95-4.90 (m, 1H), 3.61 (s, 3H), 3.32-3.22 (m, 1H), 2.51 (s, 3H), 2.30-2.25 (m, 1H), 1.28-1.23 (m, 3H), 1.10-1.02 (m, 4H). MS (ESI) calculated for (C₂₂H₂₅N₅O₂) [M+H]⁺, 392.2; found, 392.1.

### Example 289: 2-(3-(1,1,1-Trifluoro-3-(4-methyl-4H-1,2,4-triazol-3-yl)propan-2-yl)phenyl)-4-(trifluoromethyl)isoindolin-1-one (289)

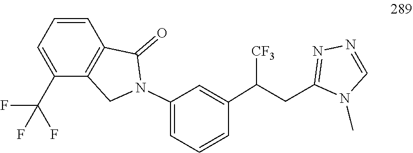 [SMILES: Cn1cnnc1CC(c1cccc(N2Cc3c(cccc3C(F)(F)F)C2=O)c1)C(F)(F)F]

Step 1: Synthesis of methyl (Z)-3-(3-bromophenyl)-4,4,4-trifluorobut-2-enoate. A solution of lithium chloride (4.42 g, 101 mmol) in acetonitrile (38.7 mL) was treated with trimethyl phosphonoacetate (4.00 mL, 24.2 mmol), 1,8-diazabicyclo[5.4.0]undec-7-ene (3.69 mL, 24.2 mmol) and 1-(3-bromophenyl)-2,2,2-trifluoro-ethanone (5.10 g, 20.2 mmol) at rt. The reaction was stirred for 4 h at reflux. The mixture was diluted water and EtOAc. The phases were separated and the aqueous layer was extracted with EtOAc (2×). The combined organic layers were washed with brine, dried, filtered and concentrated in vacuo. Purification by flash column chromatography eluting with 0-50% EtOAc/hexanes to give the title compound as a clear oil. Yield: 5.80 g (93.1%).

Step 2: Synthesis of methyl (Z)-4,4,4-trifluoro-3-(3-(1-oxo-4-(trifluoromethyl)isoindolin-2-yl)phenyl)but-2-enoate. Using methyl (Z)-3-(3-bromophenyl)-4,4,4-trifluorobut-2-enoate (0.500 g, 1.61 mmol) and 4-(trifluoromethyl)isoindolin-1-one (0.325 g, 1.61 mmol) the reaction was performed in a similar fashion to Example 277 Step 6 to give the title compound as a colorless powder. Yield: 0.466 g (67%).

Step 3: Synthesis of methyl 4,4,4-trifluoro-3-(3-(1-oxo-4-(trifluoromethyl)isoindolin-2-yl)phenyl)butanoate. A mixture of methyl (Z)-4,4,4-trifluoro-3-(3-(1-oxo-4-(trifluoromethyl)isoindolin-2-yl)phenyl)but-2-enoate (0.466 g, 1.086 mmol) and palladium on carbon (0.0466 g) was purged with nitrogen and dissolved in EtOAc/THF (1:1 v/v, 7.20 mL). The mixture was exposed to hydrogen (1 atm) and stirred at rt for 2 h. The mixture was filtered through celite and the filtrate was concentrated to dryness to provide the crude product (used without purification).

Step 4: Synthesis of 4,4,4-trifluoro-3-(3-(l-oxo-4-(trifluoromethyl)isoindolin-2-yl)phenyl)butanehydrazide. A solution of methyl 4,4,4-trifluoro-3-(3-(l-oxo-4-(trifluoromethyl)isoindolin-2-yl)phenyl)butanoate (1.059 mmol) and hydrazine (50% solution in water, 0.666 mL, 10.6 mmol) in ethanol (7.07 mL) was heated at 80° C. for 12 h. The reaction was concentrated and diluted with water. The mixture was extracted with EtOAc (2×), washed with brine, dried), filtered and evaporated to dryness. The crude was used without purification.

Step 5: Synthesis of 2-(3-(1,1,1-trifluoro-3-(5-mercapto-4-methyl-4H-1,2,4-triazol-3-yl)propan-2-yl)phenyl)-4-(trifluoromethyl)isoindolin-1-one. Using crude 4,4,4-trifluoro-3-(3-(l-oxo-4-(trifluoromethyl)isoindolin-2-yl)phenyl)butanehydrazide (1.06 mmol) the reaction was performed in a similar fashion to Example 281 Step 5 to five the title compound. The crude was used without purification.

Step 6: Synthesis of 2-(3-(1,1,1-trifluoro-3-(4-methyl-4H-1,2,4-triazol-3-yl)propan-2-yl)phenyl)-4-(trifluoromethyl)isoindolin-1-one. Using crude 2-(3-(1,1,1-trifluoro-3-(5-mercapto-4-methyl-4H-1,2,4-triazol-3-yl)propan-2-yl)phenyl)-4-(trifluoromethyl)isoindolin-1-one (1.06 mmol) the reaction was performed in a similar fashion to Example 281 Step 6 to give the title compound as an off-white solid. Yield: 0.158 g (33% over four steps). ¹H NMR (500 MHz, DMSO-d6) δ 8.31 (s, 1H), 8.14-8.02 (m, 3H), 7.88 (ddd, J=8.2, 2.3, 1.0 Hz, 1H), 7.81 (t, J=7.7 Hz, 1H), 7.44 (t, J=8.0 Hz, 1H), 7.32 (d, J=7.7 Hz, 1H), 5.22 (d, J=1.6 Hz, 2H), 4.37 (td, J=9.7, 5.2 Hz, 1H), 3.58 (s, 3H), 3.56-3.43 (m, 2H). LCMS: C₂₁H₁₆F₆N₄O requires: 454, found: m/z=455 [M+H]⁺.

### Example 290: 2-(3-(1-methoxy-2-(4-methyl-4H-1,2,4-triazol-3-yl)ethyl)phenyl)-4-(trifluoromethyl)isoindolin-1-one (290)

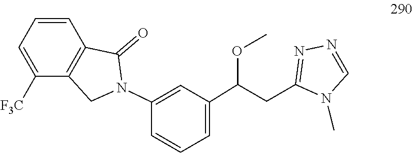 [SMILES: COC(Cc1nncn1C)c1cccc(N2Cc3c(cccc3C(F)(F)F)C2=O)c1]

Compound 290 (181.5 mg, 34%) was obtained according to procedures disclosed herein as a colorless solid. ¹H NMR (300 MHz, DMSO-d₆) δ 8.32 (s, 1H), 8.15-8.04 (m, 2H), 7.94-7.92 (m, 2H), 7.80 (t, J=7.8 Hz, 1H), 7.49-7.43 (m, 1H), 7.17 (d, J=7.8 Hz, 1H), 5.30-5.17 (m, 2H), 4.68-4.63 (m, 1H), 3.50 (s, 3H), 3.30-3.02 (m, 5H). MS (ESI) calc'd for (C₂₁H₁₉F₃N₄O₂) [M+H]⁺, 417.1; found, 417.1.

### Example 291: 2-(3-(1-ethoxy-2-(4-methyl-4H-1,2,4-triazol-3-yl)ethyl)phenyl)-4-(trifluoromethyl)isoindolin-1-one (291)

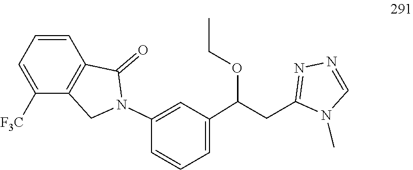 [SMILES: CCOC(Cc1nncn1C)c1cccc(N2Cc3c(cccc3C(F)(F)F)C2=O)c1]

Compound 291 (125.6 mg, 36%) was obtained according to procedures disclosed herein as a light yellow solid. ¹H NMR (300 MHz, DMSO-d₆) δ 8.32 (s, 1H), 8.15-8.04 (m, 2H), 7.94-7.83 (m, 2H), 7.80 (t, J=7.8 Hz, 1H), 7.45 (t, J=7.8 Hz, 1H), 7.18 (d, J=7.8 Hz, 1H), 5.30-5.16 (m, 2H), 4.76-4.72 (m, 1H), 3.51 (s, 3H), 3.32-3.03 (m, 4H), 1.05 (t, J=6.9 Hz, 3H). MS (ESI) calc'd for (C₂₂H₂₁F₃N₄O₂) [M+H]⁺, 431.2; found, 431.1.

### Example 292: cis-2-(3-(1-Hydroxy-1-(4-methyl-4H-1,2,4-triazol-3-yl)propan-2-yl)phenyl)-4-(trifluoromethyl)isoindolin-1-one (292)

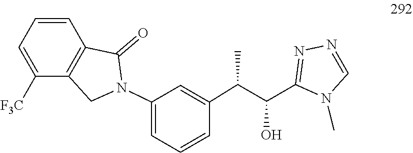 [SMILES: C[C@@H](c1cccc(N2Cc3c(cccc3C(F)(F)F)C2=O)c1)[C@@H](O)c1nncn1C]

Step 1: Synthesis of ethyl 1,3-dithiane-2-carboxylate. To a mixture of boron trifluoride diethyl etherate (230 mL) in chloroform (640 mL) was added propane-1,3-dithiol (98.3 g, 0.91 mol) dropwise at −70° C. And then to the above solution was added ethyl 2,2-diethoxyacetate (160.0 g, 0.91 mol, in chloroform (160 mL)) dropwise at 70° C. over 30 min. The resulting mixture was stirred at 70° C. for 30 min. The reaction mixture was quenched by the addition of water (1500 mL), and the aqueous layer was extracted with DCM. The combined organic layer was washed with saturated sodium bicarbonate and brine, dried over anhydrous sodium sulfate and filtered. The filtrate was concentrated under vacuum. The residue was purified by flash column chromatography with 0-15% ethyl acetate in petroleum ether to afford ethyl 1,3-dithiane-2-carboxylate (82.6 g, 47%) as a yellow oil.

Step 2: Synthesis of ethyl 2-(1-(3-nitrophenyl)ethyl)-1,3-dithiane-2-carboxylate. To a solution of ethyl 1,3-dithiane-2-carboxylate (40.1 g, 0.21 mol) in anhydrous DMF (200 mL) was added sodium hydride (10.1 g, 0.25 mol, 60% purity) in portions at 0° C. under nitrogen. The reaction mixture was stirred at 25° C. for 1.5 h. Then a solution of l-(bromomethyl)-3-nitrobenzene (45.2 g, 0.21 mol) in DMF (200 mF) was added dropwise to the above mixture at about 25° C. during 1 h. The mixture was stirred at 25° C. for 16 h, and then quenched by the addition of water. The mixture was extracted with ethyl acetate, washed with brine, dried over anhydrous sodium sulfate and filtered. The filtrate was concentrated under reduced pressure. The residue was purified by flash column chromatography with 5-34% of ethyl acetate in petroleum ether to afford ethyl 2-(1-(3-nitrophenyl)ethyl)-1,3-dithiane-2-carboxylate (40.0 g, 56%) as a yellow oil. MS (ESI) calculated for (C₁₅H₁₉NO₄S₂) [M+H]⁺, 342.1; found, 342.2.

Step 3: Synthesis of ethyl 3-(3-nitrophenyl)-2-oxobutanoate. To a mixture of ethyl 2-(1-(3-nitrophenyl)ethyl)-1,3-dithiane-2-carboxylate (20.1 g, 58.94 mmol) in acetonitrile (500 mL) and water (125 mL) was added N-bromosuccinimide (62.6 g, 0.35 mol) in portions at 0° C. The reaction mixture was stirred at 25° C. for 1.5 h, and then quenched by the addition of aqueous saturated sodium sulfite (250 mL). The mixture was extracted with ethyl acetate, washed with brine, dried over sodium sulfate and filtered. The filtrate was concentrated under vacuum. The residue was purified by flash column chromatography with 10-35% ethyl acetate in petroleum ether to afford ethyl 3-(3-nitrophenyl)-2-oxobutanoate (8.0 g, 54%) as a yellow oil.

Step 4: Synthesis of ethyl 3-(3-aminophenyl)-2-hydroxybutanoate. To a solution of ethyl 3-(3-nitrophenyl)-2-oxobutanoate (1.8 g, 7.21 mmol) in ethanol (50 mL) was added palladium on carbon (0.5 g, 10%, dry). The reaction mixture was stirred at 25° C. for 36 h under hydrogen (2 atm) atmosphere. The reaction mixture was monitored by LCMS. After the reaction was completed, the mixture was filtered. The filtrate was concentrated under reduced pressure to afford ethyl 3-(3-aminophenyl)-2-hydroxybutanoate (800 mg, 56%) as a racemic mixture of cis-isomers as a colorless oil. MS (ESI) calculated for (C₁₂H₁₇NO₃) [M+H]⁺, 224.1; found, 224.2.

Step 5: Synthesis of cis-ethyl 2-hydroxy-3-(3-(1-oxo-4-(trifluoromethyl)isoindolin-2-yl)phenyl)butanoate. Using cis-ethyl 3-(3-aminophenyl)-2-hydroxybutanoate (0.8 g, 3.61 mmol) and methyl 2-(bromomethyl)-3-(trifluoromethyl)benzoate (1.1 g, 3.61 mmol) the reaction was performed in a similar fashion to Example 256 to afford a mixture of cis-ethyl 2-hydroxy-3-(3-(l-oxo-4-(trifluoromethyl)isoindolin-2-yl)phenyl)butanoate (1.1 g, 80%) as a yellow oil. MS (ESI) calculated for (C₂₁H₂₀F₃NO₄) [M+H]⁺, 407.1; found, 407.2.

Step 6: Synthesis cis-2-hydroxy-3-(3-(1-oxo-4-(trifluoromethyl)isoindolin-2-yl)phenyl)butanoic acid. To a solution of cis-ethyl 2-hydroxy-3-(3-(l-oxo-4-(trifluoromethyl)isoindolin-2-yl)phenyl)butanoate (1.2 g, 2.81 mmol) in tetrahydrofuran (20 mL) and water (15 mL) was added lithium hydroxide (200 mg, 8.41 mmol) at 0° C. The resulting mixture was stirred at 25° C. for 2 h. The pH value of the mixture was adjusted to 3 with hydrochloric acid (3 M). The solids were collected by filtration and dried to afford cis-2-hydroxy-3-(3-(1-oxo-4-(trifluoromethyl)isoindolin-2-yl)phenyl)butanoic acid (800 mg, crude) as a white solid, which was used in next step without further purification. MS (ESI) calculated for (C₁₉H₁₆F₃NO₄) [M+H]⁺, 379.1; found, 379.2.

Step 7: Synthesis of cis-N-(hydrazinecarbonothioyl)-2-hydroxy-N-methyl-3-(3-(1-oxo-4-(trifluoromethyl)isoindolin-2-yl)phenyl)butanamide. Followed Step 3 of Example 288 to afford cis-N-(hydrazinecarbonothioyl)-2-hydroxy-N-methyl-3-(3-(1-oxo-4-(trifluoromethyl)isoindolin-2-yl)phenyl)butanamide (800 mg, 78% over 2 steps) as a white solid.

Step 8: Synthesis of cis-2-(3-(1-hydroxy-1-(5-mercapto-4-methyl-4H-1,2,4-triazol-3-yl)propan-2-yl)phenyl)-4-(trifluoromethyl)isoindolin-1-one. To a solution of cis-N-(hydrazinecarbonothioyl)-2-hydroxy-N-methyl-3-(3-(1-oxo-4-(trifluoromethyl)isoindolin-2-yl)phenyl)butanamide (0.5 g, 1.11 mmol) in ethanol (5.5 mF) was added sodium hydroxide (5.5 mF, 11.00 mmol, 2 M) at 25° C. The mixture was stirred at 100° C. for 1 h. After the reaction was completed, the pH value of the mixture was adjusted to 3 with hydrochloric acid (2 M). The solid was collected and dried to afford cis-2-(3-(1-hydroxy-1-(5-mercapto-4-methyl-4H-1,2,4-triazol-3-yl)propan-2-yl)phenyl)-4-(trifluoromethyl)isoindolin-1-one (250 mg, 68%) as a white solid.

Step 9: Synthesis of cis-2-(3-(1-hydroxy-1-(4-methyl-4H-1,2,4-triazol-3-yl)propan-2-yl)phenyl)-4-(trifluoromethyl)isoindolin-1-one. To a mixture of cis-2-(3-(1-hydroxy-1-(5-mercapto-4-methyl-4H-1,2,4-triazol-3-yl)propan-2-yl)phenyl)-4-(trifluoro-methyl)isoindolin-1-one (250 mg, 0.56 mmol) and sodium nitrite (400 mg, 5.61 mmol) was added nitric acid (1 M, 5.6 mF, 5.60 mmol) at 0° C. The mixture was stirred at 25° C. for 90 min. The reaction was quenched by the addition of saturated aqueous sodium bicarbonate solution, and then extracted with dichloromethane, washed with brine, dried over sodium sulfate and filtered. The filtrate was evaporated in vacuo. The residue was purified by Prep-HPLC to afford cis-2-(3-(1-hydroxy-1-(4-methyl-4H-1,2,4-triazol-3-yl)propan-2-yl)phenyl)-4-(trifluoromethyl)-isoindolin-1-one (100.0 mg, 50%) as a white solid. MS (ESI) calculated for (C₂₁H₁₉F₃N₄O₂) [M+H]⁺, 417.2; found, 417.0. ¹H NMR (400 MHz, DMSO-d₆) δ 8.21 (s, 1H), 8.08 (d, J=7.6 Hz, 1H), 8.03 (d, J=7.6 Hz, 1H), 7.80 (q, J=7.6 Hz, 1H), 7.73-7.69 (m, 2H), 7.27 (t, J=8.0 Hz, 1H), 7.01 (d, J=8.0 Hz, 1H), 5.90 (d, J=6.0 Hz, 1H), 5.15 (s, 2H), 4.90 (dd, J=8.4, 6.0 Hz, 1H), 3.60 (s, 3H), 3.44-3.40 (m, 1H), 1.44 (d, J=7.0 Hz, 3H). ¹⁹F NMR (400 MHz, DMSO-d₆) δ-59.76, −59.96, −59.97, −59.99.

### Example 293: trans-2-(3-(1-fluoro-1-(4-methyl-4H-1,2,4-triazol-3-yl)propan-2-yl)phenyl)-4-(trifluoromethyl)isoindolin-1-one (293)

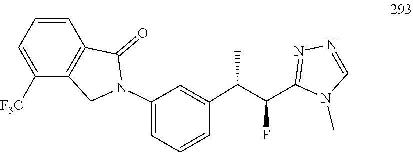 [SMILES: C[C@@H](c1cccc(N2Cc3c(cccc3C(F)(F)F)C2=O)c1)[C@H](F)c1nncn1C]

To a solution of cis-2-(3-(1-hydroxy-1-(4-methyl-4H-1,2,4-triazol-3-yl)propan-2-yl)phenyl)-4-(trifluoromethyl)isoindolin-1-one 292 (40 mg, 0.10 mmol) in anhydrous DCM (2 mL) was added DAST (124 mg, 0.77 mmol) at −78° C. The mixture was warmed slowly to rt and stirred for 16 h. The reaction was then quenched by saturated aqueous sodium bicarbonate and extracted with DCM. The organic layers were dried, filtered and concentrated under reduced pressure. The residue was purified by prep-HPLC to afford the title compound (12.4 mg, 31%) as a colorless solid. MS (ESI) calc'd for (C₂₁H₁₈F₄N₄O) [M+H]⁺, 419.1; found, 419.3. ¹H NMR (300 MHz, DMSO-d₆) δ 8.40 (s, 1H), 8.09-8.02 (m, 2H), 7.82-7.77 (m, 3H), 7.36-7.31 (m, 1H), 7.12 (d, J=7.8 Hz, 1H), 6.03 (dd, J=46.9, 8.0 Hz, 1H), 5.17 (s, 2H), 3.90-3.64 (m, 1H), 3.58 (s, 3H), 1.52 (d, J=6.0 Hz, 3H). ¹⁹F NMR (300 MHz, DMSO-d₆) δ-60.01, −180.53.

### Example 294: 2-(3-(1,1-difluoro-1-(4-methyl-4H-1,2,4-triazol-3-yl)propan-2-yl)phenyl)-4-(trifluoromethyl)isoindolin-1-one (294)

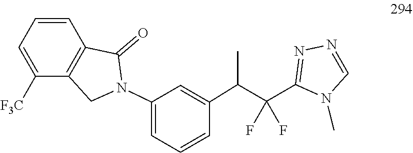 [SMILES: CC(c1cccc(N2Cc3c(cccc3C(F)(F)F)C2=O)c1)C(F)(F)c1nncn1C]

Step 1: Synthesis of 3-(3-nitrophenyl)-2-oxobutanehydrazide. To a stirring solution of ethyl 3-(3-nitrophenyl)-2-oxobutanoate (8.1 g, 31.87 mmol, step 3, synthesis of 292) in methanol (80 mF) was added hydrazine (18.0 g, 10 equiv.) dropwise at 0° C. The solution was stirred at rt for 3 h, and then concentrated. The residue was purified by flash column chromatography with 0-9% methanol in DCM to afford the title compound (6.1 g, 80%) as a yellow solid. MS (ESI) calc'd for (C₁₀H₁₁N₃O₄) [M+H]⁺, 238.1; found, 238.2.

Step 2: Synthesis of A-methyl-2-(3-(3-nitrophenyl)-2-oxobutanoyl)hydrazinecarbothioamide. To a solution of 3-(3-nitrophenyl)-2-oxobutanehydrazide (6.1 g, 25.63 mmol) in THF (250 mF) was added isothiocyanatomethane (2.3 g, 31.51 mmol). The solution was stirred at 55° C. for 2 h, and then concentrated. The residue was purified by flash column chromatography with 0-5% methanol in DCM to afford the title compound (7.5 g, 92%) as a yellow syrup. MS (ESI) calc'd for (C₁₂H₁₄N₄O₄S) [M+H]⁺, 311.1; found, 311.1.

Step 3: Synthesis of l-(5-mercapto-4-methyl-4H-1,2,4-triazol-3-yl)-2-(3-nitrophenyl)propan-1-one. A solution of N-methyl-2-(3-(3-nitrophenyl)-2-oxobutanoyl)hydrazinecarbothioamide (7.5 g, 24.19 mmol) in sodium hydroxide (1 N, 242 mF) was stirred at 45° C. for 3 h. The pH value was adjusted to 3 by the addition of hydrochloric acid (3 N). The solution was extracted with DCM. All the organic layers were combined, washed with brine, dried, and concentrated. The residue was purified by flash column chromatography with 0-7% methanol in DCM to afford the title compound (4.0 g, 56%) as a yellow solid. MS (ESI) calc'd for (C₁₂H₁₂N₄O₃S) [M+H]⁺, 293.1; found, 293.1.

Step 4: Synthesis of l-(4-methyl-4H-1,2,4-triazol-3-yl)-2-(3-nitrophenyl)propan-1-one. Using 1-(5-mercapto-4-methyl-4H-1,2,4-triazol-3-yl)-2-(3-nitrophenyl)propan-1-one (4.0 g, 13.70 mmol) the reaction was performed in a similar fashion to Example 281 Step 6 to afford the title compound (1.8 g, 51%) as a brown syrup. MS (ESI) calc'd for (C₁₂H₁₂N₄O₃) [M+H]⁺, 261.1; found, 261.2.

Step 5: Synthesis of 3-(1,1-difluoro-2-(3-nitrophenyl)propyl)-4-methyl-4H-1,2,4-triazole. A mixture of 1-(4-methyl-4H-1,2,4-triazol-3-yl)-2-(3-nitrophenyl)propan-1-one (1.8 g, 6.92 mmol) and DAST (15 mL) was stirred at 60° C. for 6 h. the reaction was quenched by the addition of aqueous saturated sodium bicarbonate at 0° C. The solution was extracted with dichloromethane. All the organic layers were combined, washed with brine, dried, and concentrated. The residue was purified by flash column chromatography with 0-20% methanol in dichloromethane then by Prep-HPLC to afford the title compound (330 mg, 17%) as a colorless solid. MS (ESI) calc'd for (C₁₂H₁₂F₂N₄O₂) [M+H]⁺, 283.1; found, 283.1.

Step 6: Synthesis of 3-(1,1-difluoro-1-(4-methyl-4H-1,2,4-triazol-3-yl)propan-2-yl)aniline. To a solution of 3-(1,1-difluoro-2-(3-nitrophenyl)propyl)-4-methyl-4H-1,2,4-triazole (150 mg, 0.53 mmol) in methanol (5 mL) was added palladium on carbon (10%, 50 mg). The solution was stirred at 25° C. for 16 h under hydrogen (2 atm). The solids were removed by filtration. The mixture was concentrated to afford the title compound (120.2 mg, crude) as a yellow syrup, which was used without purification. MS (ESI) calc'd for (C₁₂H₁₄F₂N₄) [M+H]⁺, 253.1; found, 253.2.

Step 7: Synthesis of 2-(3-(1,1-difluoro-1-(4-methyl-4H-1,2,4-triazol-3-yl)propan-2-yl)phenyl)-4-(trifluoromethyl)isoindolin-1-one. Using 3-(1,1-difluoro-1-(4-methyl-4H-1,2,4-triazol-3-yl)propan-2-yl)aniline (45.1 mg, 0.18 mmol) and methyl 2-(bromomethyl)-3-(trifluoromethyl)benzoate (56.1 mg, 0.21 mmol) the reaction was performed in a similar fashion to Example 256 to afford the title compound (9.1 mg, 26%) as a colorless solid. ¹H NMR (300 MHz, DMSO-d₆) δ 8.59 (s, 1H), 8.11-8.04 (m, 2H), 7.97 (s, 1H), 7.87-7.78 (m, 2H), 7.43 (t, J=8.1 Hz, 1H), 7.38 (d, J=7.5 Hz, 1H), 5.21 (s, 2H), 4.10-4.09 (m, 1H), 3.60 (s, 3H), 1.53 (d, J=6.6 Hz, 3H). ¹⁹F NMR (282 MHz, DMSO) 5-59.64, −60.00, −95.22, −96.17, −100.02, −100.98. MS (ESI) calc'd for (C₂₁H₁₇F₅N₄O) [M+H]⁺, 437.1; found, 436.9.

### Example 295: 2-(3-(1,1-difluoro-2-hydroxy-1-(4-methyl-4H-1,2,4-triazol-3-yl)propan-2-yl)phenyl)-4-(trifluoromethyl)isoindolin-1-one (295)

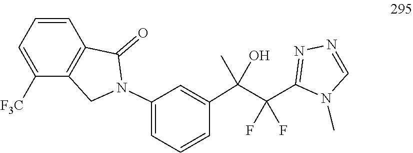 [SMILES: Cn1cnnc1C(F)(F)C(C)(O)c1cccc(N2Cc3c(cccc3C(F)(F)F)C2=O)c1]

Compound 295 (22.2 mg, 26%) was obtained according to procedures disclosed herein as a colorless solid. ¹H NMR (400 MHz, DMSO-d₆) δ 8.59 (s, 1H), 8.10-8.04 (m, 3H), 7.83-7.79 (m, 2H), 7.44 (t, J=8.0 Hz, 1H), 7.33 (d, J=8.0 Hz, 1H), 6.53-6.34 (m, 1H), 5.20 (s, 2H), 3.62 (s, 3H), 1.72 (s, 3H). ¹⁹F NMR (376 MHz, DMSO-d₆) δ-59.96, −74.76, −102.96, −103.66, −105.09, −105.80. MS (ESI) calc'd for (C₂₁H₁₇F₅N₄O₂) [M+H]⁺, 453.1; found, 453.2.

### Example 296: 2-(3-(1,1,2-trifluoro-1-(4-methyl-4H-1,2,4-triazol-3-yl)propan-2-yl)phenyl)-4-(trifluoromethyl)isoindolin-1-one (296)

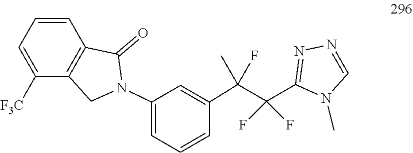 [SMILES: Cn1cnnc1C(F)(F)C(C)(F)c1cccc(N2Cc3c(cccc3C(F)(F)F)C2=O)c1]

To a stirring solution of 2-(3-(1,1-difluoro-2-hydroxy-1-(4-methyl-4H-1,2,4-triazol-3-yl)propan-2-yl)phenyl)-4-(trifluoromethyl)isoindolin-1-one 295 (120.0 mg, 0.27 mmol) in DCM (5.0 mL), was added DAST (434.1 mg, 2.70 mmol) dropwise at 0° C. The solution was stirred at 25° C. for 16 h. The reaction was then quenched by the addition of aqueous saturated sodium bicarbonate and extracted with DCM (3×50 mL). The combined organic layers were washed with brine, dried, and concentrated. The residue was purified by Prep-HPLC to afford the title compound (24.0 mg, 20%) as colorless solid. ¹H NMR (400 MHz, DMSO-d₆) δ 8.63 (s, 1H), 8.10 (d, J=7.6 Hz, 1H), 8.07-8.04 (m, 2H), 7.93-7.90 (m, 1H), 7.81 (t, J=7.8 Hz, 1H), 7.52 (t, J=8.0 Hz, 1H), 7.22 (d, J=8.0 Hz, 1H), 5.22 (s, 2H), 3.45 (s, 3H), 1.99 (d, J=24.0 Hz, 3H). ¹⁹F NMR (376 MHz, DMSO) δ−59.98, −74.57, −90.20, −105.68, −158.16. MS (ESI) calc'd for (C₂₁H₁₆F₆N₄O) [M+H]⁺, 455.1; found, 454.9.

### (R)-2-(3-(1,1,2-trifluoro-1-(4-methyl-4H-1,2,4-triazol-3-yl)propan-2-yl)phenyl)-4-(trifluoromethyl)isoindolin-1-one (296a) and (S)-2-(3-(1,1,2-trifluoro-1-(4-methyl-4H-1,2,4-triazol-3-yl)propan-2-yl)phenyl)-4-(trifluoromethyl)isoindolin-1-one (296b)

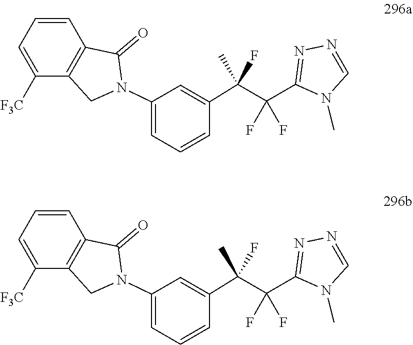 [SMILES: Cn1cnnc1C(F)(F)[C@](C)(F)c1cccc(N2Cc3c(cccc3C(F)(F)F)C2=O)c1 ; Cn1cnnc1C(F)(F)[C@@](C)(F)c1cccc(N2Cc3c(cccc3C(F)(F)F)C2=O)c1]

The racemic compound 2-(3-(1,1,2-trifluoro-1-(4-methyl-4H-1,2,4-triazol-3-yl)propan-2-yl)phenyl)-4-(trifluoromethyl)isoindolin-1-one 296 (130 mg, 85% purity) was separated by Chiral-HPLC to afford:

(R)-2-(3-(1,1,2-trifluoro-1-(4-methyl-4H-1,2,4-triazol-3-yl)propan-2-yl)phenyl)-4-(trifluoromethyl)isoindolin-1-one (shorter retention, 28.7 mg, colorless solid): MS (ESI) calc'd for (C₂₁H₁₆F₆N₄O) [M+H]⁺, 455.1; found, 454.8. ¹H NMR (300 MHz, DMSO-d₆) δ 8.63 (s, 1H), 8.12-8.05 (m, 3H), 7.94-7.90 (m, 1H), 7.82 (t, J=7.8 Hz, 1H), 7.53 (t, J=8.1 Hz, 1H), 7.23 (d, J=8.1 Hz, 1H), 5.22 (s, 2H), 3.46 (s, 3H), 2.00 (d, J=24.3 Hz, 3H). ¹⁹F NMR (282 MHz, DMSO) δ-59.98, −74.57, −90.20, −105.68, −158.16.

(S)-2-(3-(1,1,2-trifluoro-1-(4-methyl-4H-1,2,4-triazol-3-yl)propan-2-yl)phenyl)-4-trifluoromethyl)isoindolin-1-one (longer retention, 22.9 mg, colorless solid): MS (ESI) calc'd for (C₂₁H₁₆F₆N₄O) [M+H]⁺, 455.1; found, 454.8. ¹H NMR (300 MHz, DMSO-d₆) δ 8.63 (s, 1H), 8.12-8.05 (m, 3H), 7.94-7.90 (m, 1H), 7.82 (t, J=7.8 Hz, 1H), 7.53 (t, J=8.1 Hz, 1H), 7.23 (d, J=8.4 Hz, 1H), 5.22 (s, 2H), 3.46 (s, 3H), 2.00 (d, J=24.3 Hz, 3H). ¹⁹F NMR (282 MHz, DMSO) δ-59.98, −74.57, −90.20, −105.68, −158.16.

### Example 297: 2-(3-(2-hydroxy-1-(4-methyl-4H-1,2,4-triazol-3-yl)propan-2-yl)phenyl)-4-(trifluoromethyl)isoindolin-1-one (297)

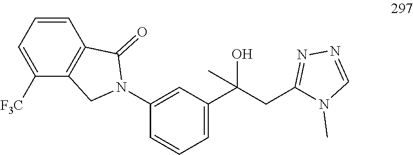 [SMILES: Cn1cnnc1CC(C)(O)c1cccc(N2Cc3c(cccc3C(F)(F)F)C2=O)c1]

Compound 297 (32.0 mg, 1.8%) was obtained according to procedures disclosed herein as a colorless solid. ¹H NMR (400 MHz, DMSO-d₆) δ 8.30 (s, 1H), 8.10-8.04 (m, 2H), 7.97 (t, J=2.0 Hz, 1H), 7.85-7.79 (m, 2H), 7.39 (t, J=8.0 Hz, 1H), 7.29 (t, J=8.0 Hz, 1H), 5.51 (s, 1H), 5.26-5.16 (m, 2H), 3.44 (s, 3H), 3.19-3.11 (m, 2H), 1.56 (s, 3H). MS (ESI) calc'd for (C₂₁H₁₉F₃N₄O₂) [M+H]⁺, 417.2; found, 417.1.

### Example 298: 2-(3-(2-fluoro-1-(4-methyl-4H-1,2,4-triazol-3-yl)propan-2-yl)phenyl)-4-(trifluoromethyl)isoindolin-1-one (298)

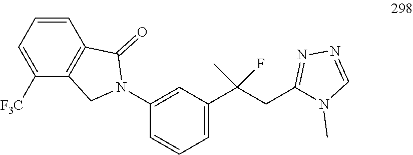 [SMILES: Cn1cnnc1CC(C)(F)c1cccc(N2Cc3c(cccc3C(F)(F)F)C2=O)c1]

To a solution of 2-[3-[2-hydroxy-1-(4-methyl-4H-1,2,4-triazol-3-yl)propan-2-yl]phenyl]-4-(trifluoromethyl)isoindolin-1-one (300 mg, 0.72 mmol) in DCM (5.0 mL) was added DAST (232.0 mg, 1.44 mmol) at −78° C. The mixture was warmed to rt and stirred for 2 h. The reaction was then quenched by the addition of aqueous sodium bicarbonate. The combined organic layers were washed with brine, dried, and filtered. The filtrate was concentrated. The product was purified by Prep-HPLC to afford the title compound (60.4 mg, 20%) as a colorless solid. ¹H NMR (300 MHz, DMSO-d₆) δ 8.35 (s, 1H), 8.11-8.04 (m, 2H), 7.94-7.90 (m, 2H), 7.80 (t, J=7.8 Hz, 1H), 7.44 (t, J=7.8 Hz, 1H), 7.24 (d, J=7.5 Hz, 1H), 5.24 (s, 2H), 3.63-3.46 (m, 5H), 1.74 (d, J=23.1 Hz, 3H). ¹⁹F NMR (300 MHz, DMSO-d₆) δ-59.98, −142.44. MS (ESI) calc'd for (C₂₁H₁₈F₄N₄O) [M+H]⁺, 419.1; found, 419.1.

### (R)-2-(3-(2-fluoro-1-(4-methyl-4H-1,2,4-triazol-3-yl)propan-2-yl)phenyl)-4-(trifluoromethyl)isoindolin-1-one (298a) and (S)-2-(3-(2-fluoro-1-(4-methyl-4H-1,2,4-triazol-3-yl)propan-2-yl)phenyl)-4-(trifluoromethyl)isoindolin-1-one (298b)

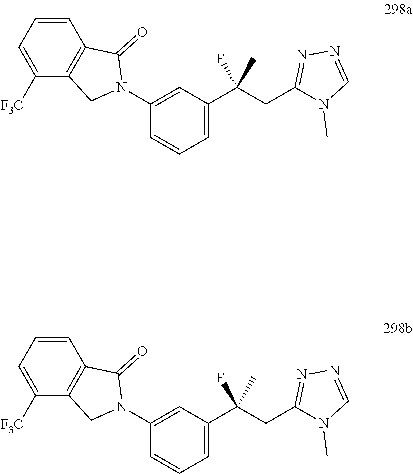 [SMILES: Cn1cnnc1C[C@@](C)(F)c1cccc(N2Cc3c(cccc3C(F)(F)F)C2=O)c1 ; Cn1cnnc1C[C@](C)(F)c1cccc(N2Cc3c(cccc3C(F)(F)F)C2=O)c1]

The racemic compound 2-[3-[2-fluoro-1-(4-methyl-4H-1,2,4-triazol-3-yl)propan-2-yl]phenyl]-4-(trifluoromethyl)isoindolin-1-one 298 (50 mg) was separated by Chiral-Prep-HPLC to afford:

(R)-2-(3-(2-fluoro-1-(4-methyl-4H-1,2,4-triazol-3-yl)propan-2-yl)phenyl)-4-(trifluoromethyl)isoindolin-1-one (shorter retention, 17.8 mg, colorless solid): MS (ESI) calc'd for (C₂₁H₁₈F₄N₄O) [M+H]⁺, 419.1; found, 418.9. ¹H NMR (300 MHz, DMSO-d₆) δ 8.36 (s, 1H), 8.11-8.04 (m, 2H), 7.94-7.91 (m, 2H), 7.81 (t, J=7.5 Hz, 1H), 7.46 (t, J=7.8 Hz, 1H), 7.23 (d, J=7.8 Hz, 1H), 5.24 (s, 2H), 3.59-3.45 (m, 5H), 1.77 (d, J=23.1 Hz, 3H). ¹⁹F NMR (300 MHz, DMSO-d₆) δ-59.974, −142.331.

(S)-2-(3-(2-fluoro-1-(4-methyl-4H-1,2,4-triazol-3-yl)propan-2-yl)phenyl)-4-(trifluoromethyl)isoindolin-1-one (longer retention, 24.9 mg, colorless solid): MS (ESI) calc'd for (C₂₁H₁₈F₄N₄O) [M+H]⁺, 419.1; found, 418.9. ¹H NMR (300 MHz, DMSO-d₆) δ 8.36 (s, 1H), 8.11-8.04 (m, 2H), 7.94-7.91 (m, 2H), 7.81 (t, J=7.5 Hz, 1H), 7.46 (t, J=7.8 Hz, 1H), 7.23 (d, J=7.8 Hz, 1H), 5.24 (s, 2H), 3.59-3.45 (m, 5H), 1.77 (d, J=23.1 Hz, 3H). ¹⁹F NMR (300 MHz, DMSO-d₆) δ-59.975, −142.325.

### Example 299: 2-(3-(1-((4-methyl-4H-1,2,4-triazol-3-yl)methyl)cyclopropyl)phenyl)-4-(trifluoromethyl)isoindolin-1-one (299)

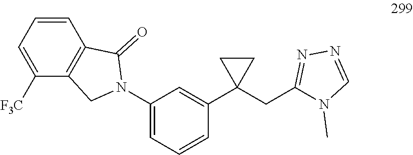 [SMILES: Cn1cnnc1CC1(c2cccc(N3Cc4c(cccc4C(F)(F)F)C3=O)c2)CC1]

Step 1 Synthesis of l-bromo-3-[1-(bromomethyl)cyclopropyl]benzene. To a solution of [1-(3-bromophenyl)cyclopropyl]methanol (WO2014201073) (10.0 g, 44.03 mmol) in DCM (100 mL) was added carbon tetrabromide (22.0 g, 66.34 mmol) and triphenylphosphine (17.4 g, 66.34 mmol). The mixture was stirred at rt for 16 h. The mixture was concentrated. The residue was purified by flash column chromatography with 0-10% EtOAc in petroleum ether to afford the title compound (6.5 g, 51%) as a light yellow oil.

Step 2: Synthesis of 2-[1-(3-bromophenyl)cyclopropyl]acetonitrile. To a solution of l-bromo-3-[1-(bromomethyl)cyclopropyl]benzene (6.0 g, 20.69 mmol) in DMSO (60 mL) was added sodium cyanide (3.1 g, 62.07 mmol). The mixture was stirred at 60° C. for 3 h. The mixture was diluted with water, followed by General Work-up Procedure 1 to afford the title compound (5.0 g, crude) as a light red oil, which was used without purification.

Step 3: Synthesis of 2-[1-(3-bromophenyl)cyclopropyl]acetic acid. To a solution of 2-[1-(3-bromophenyl)cyclopropyl]acetonitrile (5.0 g, 21.18 mmol) in ethanol (50 mL) and water (10 mL) was added potassium hydroxide (11.9 g, 212.08 mmol). The mixture was stirred at 80° C. for 16 h. The mixture was acidified with hydrochloric acid (conc.) to pH=6, followed by General Work-up Procedure 1 to afford the title compound (5.2 g, 96%) as a light brown solid.

Step 4: Synthesis of 2-[1-(3-bromophenyl)cyclopropyl]-N-[(methylcarbamothioyl)amino]acetamide. To a mixture of 1-amino-3-methylthiourea (2.3 g, 21.59 mmol) and 2-[1-(3-bromophenyl)cyclopropyl]acetic acid (5.0 g, 19.60 mmol) in DMF (50 mL) were added EDC (4.5 g, 23.63 mmol), HOBt (3.5 g, 25.53 mmol) and 4-methylmorpholine (19.9 g, 196.74 mmol). The mixture was stirred at rt for 16 h. The mixture was diluted with water, followed by General Work-up Procedure 1 to afford the title compound (5.8 g, 86%, crude) as a yellow solid, which was used without purification. MS (ESI) calc'd for (C₁₃H₁₆BrN₃OS) [M+H]⁺, 342.0; found, 342.0.

Step 5: Synthesis of 5-[[1-(3-bromophenyl)cyclopropyl]methyl]-4-methyl-4H-1,2,4-triazole-3-thiol. To a solution of 2-[1-(3-bromophenyl)cyclopropyl]-N-[(methylcarbamothioyl)amino]acetamide (5.8 g, 16.95 mmol) in THE (40 mL) was added aqueous sodium hydroxide (40 mL, 1 M). The mixture was stirred at rt for 5 h. The mixture was acidified with hydrochloric acid (conc.) to pH=6, followed by General Work-up Procedure 1. The residue was purified by flash column chromatography with 0-60% EtOAc in petroleum ether to afford the title compound (2.9 g, 53%) as a light yellow solid. MS (ESI) calc'd for (C₁₃H₁₄BrN₃S) [M+H]⁺, 324.0; found, 324.0.

Step 6: Synthesis of 3-((1-(3-bromophenyl)cyclopropyl)methyl)-4-methyl-4H-1,2,4-triazole. To a solution of 5-[[1-(3-bromophenyl)cyclopropyl]methyl]-4-methyl-4H-1,2,4-triazole-3-thiol (2.9 g, 8.94 mmol) in acetic acid (10 mL) and dichloromethane (20 mL) was added hydrogen peroxide (3.1 g, 26.82 mmol, 30%). The mixture was stirred at rt for 1 h. The mixture was diluted with water, followed by General Work-up Procedure 1. The residue purified by flash column chromatography with 0-40% EtOAc in petroleum ether to afford the title compound (2.0 g, 77%) as light yellow oil. MS (ESI) calc'd for (C₁₃H₁₄BrN₃) [M+H]⁺, 324; found, 324.

Step 7: Synthesis of 2-(3-[1-[(4-methyl-4H-1,2,4-triazol-3-yl)methyl]cyclopropyl]phenyl)-4-(trifluoromethyl)isoindolin-1-one. Using 3-[[1-(3-bromophenyl)cyclopropyl]methyl]-4-methyl-4H-1,2,4-triazole (500 mg, 1.71 mmol) and 4-(trifluoromethyl)isoindolin-1-one (345 mg, 1.72 mmol) the reaction was performed in a similar fashion to Example 277 Step 6 to afford the title compound (73.5 mg, 10%) as a colorless solid. ¹H NMR (300 MHz, DMSO-d₆) δ 8.24 (s, 1H), 8.15-8.02 (m, 2H), 7.82-7.75 (m, 3H), 7.32 (t, J=7.8 Hz, 1H), 7.05-7.03 (m, 1H), 5.15 (s, 2H), 3.24 (s, 3H), 3.11 (s, 2H), 0.97-0.87 (m, 4H). MS (ESI) calc'd for (C₂₂H₁₉F₃N₄O) [M+H]⁺, 413.1; found, 412.9.

### Example 300: 2-(3-(2-methyl-1-(4-methyl-4H-1,2,4-triazol-3-yl)propan-2-yl)phenyl)-4-(trifluoromethyl)isoindolin-1-one (300)

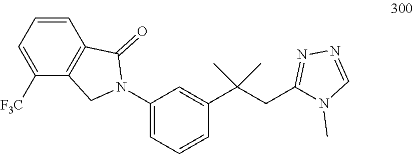 [SMILES: Cn1cnnc1CC(C)(C)c1cccc(N2Cc3c(cccc3C(F)(F)F)C2=O)c1]

Using 3-(2-(3-bromophenyl)-2-methylpropyl)-4-methyl-4H-1,2,4-triazole (200 mg, 0.68 mmol, step 6, intermediate I) and 4-(trifluoromethyl)isoindolin-1-one (136.7 mg, 0.68 mmol) the reaction was performed in a similar fashion to Example 277 Step 6 to afford the title compound (49.3 mg, 17%) as a colorless solid. ¹H NMR (300 MHz, DMSO-d₆) δ 8.23 (s, 1H), 8.10-8.03 (m, 2H), 7.90 (s, 1H), 7.84-7.74 (m, 2H), 7.40-7.33 (m, 1H), 7.14 (d, J=8.1 Hz, 1H), 5.19 (s, 2H), 3.15 (s, 3H), 3.02 (s, 2H), 1.46 (s, 3H), 1.43 (s, 3H). MS (ESI) calculated for (C₂₂H₂₁F₃N₄O) [M+H]⁺, 415.2; found, 415.0.

### Example 301: 2-[3-[3-[(4-methyl-1,2,4-triazol-3-yl)methyl]oxetan-3-yl]phenyl]-4-(trifluoromethyl)isoindolin-1-one (301)

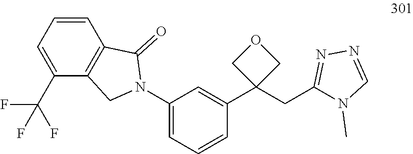 [SMILES: Cn1cnnc1CC1(c2cccc(N3Cc4c(cccc4C(F)(F)F)C3=O)c2)COC1]

Step 1: Ethyl 2-[3-[3-[1-oxo-4-(trifluoromethyl)isoindolin-2-yl]phenyl]oxetan-3-yl]acetate. Followed Example 168, step 1 using ethyl 2-[3-(3-bromophenyl)oxetan-3-yl]acetate (WO 2009073300) to give the title compound (700 mg, 1.72 mmol, 41% yield).

Step 2: 2-(3-(3-(l-oxo-4-(trifluoromethyl)isoindolin-2-yl)phenyl)oxetan-3-yl)acetohydrazide. To a stirring solution of ethyl 2-[3-[3-[1-oxo-4-(trifluoromethyl)isoindolin-2-yl]phenyl]oxetan-3-yl]acetate (200 mg, 0.48 mmol, 1 eq.) in ethanol (4.8 mL) was added hydrazine (0.3 mL, 4.77 mmol, 10 eq.). The mixture was stirred at 80° C. for about 12 h, cooled to rt, and then concentrated. The reaction was diluted with water, followed by General Work-up Procedure 1. The crude was used without purification.

Step 3: 2-[3-[3-[(4-methyl-5-sulfanyl-1,2,4-triazol-3-yl)methyl]oxetan-3-yl]phenyl]-4-(trifluoromethyl)isoindolin-1-one. To a stirring solution of 2-[3-[3-[1-oxo-4-(trifluoromethyl)isoindolin-2-yl]phenyl]oxetan-3-yl]acetohydrazide (193 mg, 0.48 mmol, 1 eq.) in THF (2.4 mL) was added methyl isocyanate (0.1 mL, 1.43 mmol, 3 eq.). The mixture was stirred at rt for about 16 h. 2 M potassium hydroxide (2.4 mL) was added and the solution was stirred for 2 h, followed by General Work-up Procedure 1. The crude was used without purification.

Step 4: 2-[3-[3-[(4-methyl-1,2,4-triazol-3-yl)methyl]oxetan-3-yl]phenyl]-4-(trifluoromethyl) isoindolin-1-one. Using 2-[3-fluoro-5-[1-methyl-2-(4-methyl-5-sulfanyl-1,2,4-triazol-3-yl)ethyl]phenyl]-4-(trifluoromethyl)isoindolin-1-one the reaction was performed in a similar fashion to Example 281 Step 6 to give the title compound (25 mg, 0.06 mmol, 12% yield) as a colorless solid. 1H NMR (500 MHz, methanol-d4) δ 8.18 (s, 1H), 8.08 (d, J=7.7 Hz, 1H), 7.97 (d, J=7.7 Hz, 1H), 7.81-7.72 (m, 2H), 7.43-7.36 (m, 2H), 6.78-6.70 (m, 1H), 5.10 (s, 2H), 5.07 (q, J=6.1 Hz, 4H), 3.65 (s, 3H), 2.89 (s, 2H). LCMS: C21H18F4N4O requires: 428.4, found: m/z=429.4 [M+H]⁺.

### Example 302: (R)-N-(4-(1-(4-methyl-4H-1,2,4-triazol-3-yl)propan-2-yl)pyridin-2-yl)-6-(trifluoromethyl)picolinamide (302)

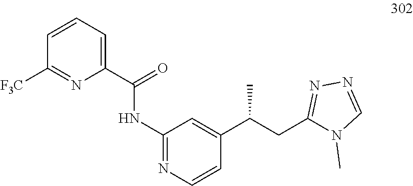 [SMILES: C[C@H](Cc1nncn1C)c1ccnc(NC(=O)c2cccc(C(F)(F)F)n2)c1]

Step 1: Synthesis of ethyl (E)-3-(2-((tert-butoxycarbonyl)amino)pyridin-4-yl)but-2-enoate. To a stirred solution of ethyl 2-(diethoxyphosphoryl)acetate (77.8 g, 347 mmol) in THF (2000 mL) was added potassium tert-butoxide (38.9 g, 347 mmol) in batches at 0° C. under nitrogen. After 30 min, a solution of tert-butyl (4-acetylpyridin-2-yl)carbamate (20.0 g, 84.7 mmol) in THF (100 mL) was added dropwise. The mixture was stirred at 20° C. for 16 h. When the reaction was complete, the solution was diluted with EtOAc (2000 mL) and washed with brine (2000 mL). The organic solution was dried, filtered, and concentrated. The residue was purified by flash column chromatography with 20-50% EtOAc in n-hexane to afford the title compound (25.0 g, 24%) as a colorless solid. MS (ESI) calculated for (C₁₆H₂₂N₂O₄) [M+H]⁺, 307.1; found, 307.1.

Step 2: Synthesis of ethyl 3-(2-((tert-butoxycarbonyl)amino)pyridin-4-yl)butanoate. To a solution of (E)-3-(2-((tert-butoxycarbonyl)amino)pyridin-4-yl)but-2-enoate (32.8 g, 107.0 mmol) in ethanol (1000 mL) and THF (500 mL) was added palladium on carbon (26.0 g, wet, 10%). The mixture was degassed and stirred at 20° C. for 16 h under hydrogen. When the reaction was complete, the mixture was filtered and concentrated. The residue was purified by flash column chromatography with 20-50% EtOAc in n-hexanes to afford the title compound (28.0 g, 85%) as a colorless solid. MS (ESI) calculated for C₁₆H₂₄N₂O₄) [M+H]⁺, 309.1; found, 309.1.

Step 3: Synthesis of tert-butyl (4-(4-hydrazineyl-4-oxobutan-2-yl)pyridin-2-yl)carbamate. To a solution of ethyl 3-(2-((tert-butoxycarbonyl)amino)pyridin-4-yl)butanoate (28.0 g, 90.90 mmol) in ethanol (500 mL) was added hydrazine (68.2 g, 1.36 mol). The solution was stirred at 80° C. for 16 h. When the reaction was complete, the reaction was quenched by the addition of water (1000 mL), followed by General Work-up Procedure 1 to give the title compound (28.0 g, crude) as a colorless solid. MS (ESI) calculated for (C₁₄H₂₂N₄O₃) [M+H]⁺, 295.1; found, 295.1.

Step 4: Synthesis of tert-butyl (E)-(4-(4-(2-((dimethylamino)methylene)hydrazineyl)-4-oxobutan-2-yl)pyridin-2-yl)carbamate. To a solution of tert-butyl (4-(4-hydrazineyl-4-oxobutan-2-yl)pyridin-2-yl)carbamate (28.0 g, crude) in DCM (350 mL) was added dimethylformamide dimethyl acetal (17.0 g, 143.00 mmol). The solution was stirred at 40° C. for 2 h. When the reaction was complete, the mixture was concentrated under reduced pressure. The residue was purified by flash column chromatography with 0-10% methanol in DCM to afford the title compound (26.0 g, 78%) as a colorless solid: MS (ESI) calculated for (C₁₇H₂₇N₅O₃) [M+H]⁺, 350.1; found, 350.1.

Step 5: Synthesis of tert-butyl (4-(1-(4-methyl-4H-1,2,4-triazol-3-yl)propan-2-yl)pyridin-2-yl)carbamate. To a solution of tert-butyl (E)-(4-(4-(2-((dimethylamino)methylene)hydrazineyl)-4-oxobutan-2-yl)pyridin-2-yl)carbamate (1.0 g, 2.86 mmol) in acetic acid (10 mL) was added methylamine (6 mL, 11.50 mmol, 2 M in THF). The solution was stirred at 80° C. for 2 h. The reaction was quenched by the addition of 20 mL water. The aqueous solution was basified with saturated sodium bicarbonate solution to pH=9, followed by General Work-up Procedure 1, and the residue was purified by flash column chromatography with 0-10% methanol in DCM to afford the title compound (450 mg, crude) as a colorless oil, which was used without purification. MS (ESI) calculated for (C₁₆H₂₃N₅O₂) [M+H]⁺, 318.1; found, 318.1.

Step 6: Synthesis of 4-(1-(4-methyl-4H-1,2,4-triazol-3-yl)propan-2-yl)pyridin-2-amine. To a stirred solution of tert-butyl (4-(1-(4-methyl-4H-1,2,4-triazol-3-yl)propan-2-yl)pyridin-2-yl)carbamate (300 mg, 0.94 mmol) in DCM (3 mL) was added trifluoroacetic acid (1 mL). The solution was stirred at rt for 1.5 h. The solution was concentrated to give the crude product, which was purified by prep-HPLC to afford the title compound (210 mg, >99%) as a colorless solid. MS (ESI) calculated for (C₁₁H₁₅N₅) [M+H]⁺, 218.1; found, 218.1.

Step 7: Synthesis of (R)-N-(4-(1-(4-methyl-4H-1,2,4-triazol-3-yl)propan-2-yl)pyridin-2-yl)-6-(trifluoromethyl)picolinamide. Followed General procedure 1-G to afford N-(4-(1-(4-methyl-4H-1,2,4-triazol-3-yl)propan-2-yl)pyridin-2-yl)-6-(trifluoromethyl)-picolinamide (90 mg, 50%). The racemic product was separated by prep-chiral-HPLC to afford:

(S)-N-(4-(1-(4-methyl-4H-1,2,4-triazol-3-yl)propan-2-yl)pyridin-2-yl)-6-(trifluoromethyl)picolinamide (shorter retention, 36 mg, colorless semi-solid): MS (ESI) calculated for (C₁₈H₁₇F₃N₆O) [M+H]⁺, 391.2; found, 391.2. ¹H NMR (400 MHz, DMSO-d₆) δ 10.20 (s, 1H), 8.50-8.38 (m, 2H), 8.30 (d, J=4.4 Hz, 2H), 8.25-8.24 (m, 1H), 8.21-8.14 (m, 1H), 7.20-7.19 (m, 1H), 3.54 (s, 3H), 3.43-3.36 (m, 1H), 3.12-2.98 (m, 2H), 1.32 (d, J=6.8 Hz, 3H).

(R)-N-(4-(1-(4-methyl-4H-1,2,4-triazol-3-yl)propan-2-yl)pyridin-2-yl)-6-(trifluoromethyl)picolinamide (longer retention, 44 mg, colorless semi-solid): MS (ESI) calculated for (C₁₈H₁₇F₃N₆O) [M+H]⁺, 391.2; found, 391.2. ¹H NMR (400 MHz, DMSO-d₆) δ 10.20 (s, 1H), 8.48-8.44 (m, 2H), 8.30 (t, J=2.4 Hz, 2H), 8.24 (d, J=7.6 Hz, 1H), 8.20 (s, 1H), 7.20-7.19 (m, 1H), 3.54 (s, 3H), 3.38 (d, J=7.2 Hz, 1H), 3.11-2.97 (m, 2H), 1.33 (d, J=6.8 Hz, 3H).

### Example 303: (R)-N-(4-(1-(4-methyl-4H-1,2,4-triazol-3-yl)propan-2-yl)pyridin-2-yl)quinoline-2-carboxamide (303)

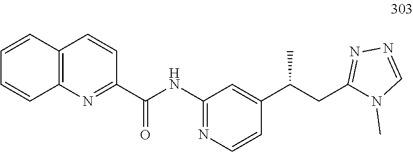 [SMILES: C[C@H](Cc1nncn1C)c1ccnc(NC(=O)c2ccc3ccccc3n2)c1]

Followed Example 302, step 7 using quinoline-2-carboxylic acid to afford:

(S)-N-(4-(1-(4-methyl-4H-1,2,4-triazol-3-yl)propan-2-yl)pyridin-2-yl)quinoline-2-carboxamide (shorter retention, 19 mg, colorless semi-solid): 1H NMR (400 MHz, DMSO-d₆) δ 10.61 (s, 1H), 8.70 (d, J=8.4 Hz, 1H), 8.36-8.26 (m, 5H), 8.16-8.15 (m, 1H), 7.96-7.94 (m, 1H), 7.80-7.79 (m, 1H), 7.20-7.19 (m, 1H), 3.55 (s, 3H), 3.44-3.36 (m, 1H), 3.14-2.99 (m, 2H), 1.34 (d, J=6.8 Hz, 3H). MS (ESI) calculated for (C₂₁H₂₀N₆O) [M+H]⁺, 373.2; found, 373.2.

(R)-N-(4-(1-(4-methyl-4H-1,2,4-triazol-3-yl)propan-2-yl)pyridin-2-yl)quinoline-2-carboxamide (longer retention, 20 mg, colorless semi-solid): ¹H NMR (400 MHz, DMSO-d₆) δ 10.61 (s, 1H), 8.70 (d, J=8.4 Hz, 1H), 8.37-8.23 (m, 5H), 8.17-8.15 (m, 1H), 7.96-7.94 (m, 1H), 7.80-7.79 (m, 1H), 7.20-7.19 (m, 1H), 3.55 (s, 3H), 3.44-3.36 (m, 1H), 3.14-2.98 (m, 2H), 1.34 (d, J=6.8 Hz, 3H). MS (ESI) calculated for (C₂₁H₂₀N₆O) [M+H]⁺, 373.2; found, 373.2.

### Example 304: (S)-2-(4-(1-(4-methyl-4H-1,2,4-triazol-3-yl)propan-2-yl)pyridin-2-yl)-4-(trifluoromethyl)isoindolin-1-one (304a) and (K)-2-(4-(1-(4-methyl-4H-1,2,4-triazol-3-yl)propan-2-yl)pyridin-2-yl)-4-(trifluoromethyl)isoindolin-1-one (304b)

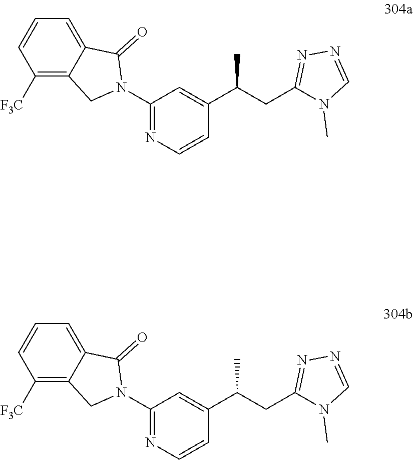 [SMILES: C[C@@H](Cc1nncn1C)c1ccnc(N2Cc3c(cccc3C(F)(F)F)C2=O)c1 ; C[C@H](Cc1nncn1C)c1ccnc(N2Cc3c(cccc3C(F)(F)F)C2=O)c1]

A mixture of 2-chloro-4-[1-(4-methyl-4H-1,2,4-triazol-3-yl)propan-2-yl]pyridine (675.0 mg, 2.85 mmol), 4-(trifluoromethyl)isoindolin-1-one (500.0 mg, 2.49 mmol), tris(dibenzylideneacetone)dipalladium(0) (531.0 mg, 0.58 mmol), XPhos (593.0 mg, 1.24 mmol) and cesium carbonate (2.5 g, 7.58 mmol) in 1,4-dioxane (25 mL) was heated to 100° C. for 16 h. The mixture was filtered. The filtrate was evaporated in vacuo. The residue was purified by reverse phase flash column chromatography to afford racemic title compound (130.0 mg, 11%) as a colorless solid. The racemic product was separated by prep-chiral-HPLC to afford:

(S)-2-(4-(1-(4-methyl-4H-1,2,4-triazol-3-yl)propan-2-yl)pyridin-2-yl)-4-(trifluoromethyl)isoindolin-1-one (shorter retention, 53 mg, colorless solid): MS (ESI) calculated for (C₂₀H₁₈F₃N₅O) [M+H]⁺, 402.1; found, 402.0. ¹H NMR (300 MHz, DMSO-d₆) δ 8.47 (s, 1H), 8.36 (d, J=5.1 Hz, 1H), 8.31 (s, 1H), 8.13-8.06 (m, 2H), 7.83-7.78 (m, 1H), 7.21-7.19 (m, 1H), 5.25 (s, 2H), 3.55 (s, 3H), 3.42-3.34 (m, 1H), 3.07-3.03 (m, 2H) 1.32 (d, J=6.9 Hz, 3H).

(R)-2-(4-(1-(4-methyl-4H-1,2,4-triazol-3-yl)propan-2-yl)pyridin-2-yl)-4-(trifluoromethyl)isoindolin-1-one (longer retention, 48 mg, colorless solid): MS (ESI) calculated for (C₂₀H₁₈F₃N₅O) [M+H]⁺, 402.1; found, 402.0. ¹H NMR (300 MHz, DMSO-d₆) δ 8.47 (s, 1H), 8.36 (d, J=5.4 Hz, 1H), 8.31 (s, 1H), 8.13-8.06 (m, 2H), 7.83-7.78 (m, 1H), 7.21-7.19 (m, 1H), 5.25 (s, 2H), 3.55 (s, 3H), 3.42-3.34 (m, 1H), 3.07-3.03 (m, 2H) 1.32 (d, J=6.9 Hz, 3H).

### Example 305: (S)-2-((3-((3-(1-((4-methyl-4H-1,2,4-triazol-3-yl)thio)ethyl)phenyl)-carbamoyl)isoquinolin-6-yl)oxy)acetic acid (305)

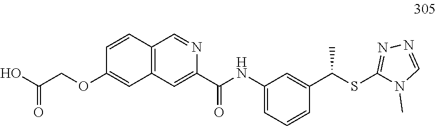 [SMILES: C[C@H](Sc1nncn1C)c1cccc(NC(=O)c2cc3cc(OCC(=O)O)ccc3cn2)c1]

Step 1: Synthesis of methyl 6-[2-(tert-butoxy)-2-oxoethoxy]isoquinoline-3-carboxylate. To a solution of methyl 6-hydroxyisoquinoline-3-carboxylate (WO2014014874) (1.45 g, 7.14 mmol) in acetonitrile (50 mL) was added potassium carbonate (1.48 g, 10.71 mmol) and tert-butyl 2-bromoacetate (1.67 g, 8.56 mmol). The mixture was stirred at 85° C. for 16 h. The mixture was cooled to rt and then filtered. The filtrate was evaporated in vacuo. The residue was purified similar to the last step to afford the title compound (1.3 g, 57%) as a colorless solid. MS (ESI) calculated for (C₁₇H₁₉NO₅) [M+H]⁺, 318.1; found, 318.1.

Step 2: Synthesis of 6-[2-(tert-butoxy)-2-oxoethoxy]isoquinoline-3-carboxylic acid. To a solution of methyl 6-[2-(tert-butoxy)-2-oxoethoxy]isoquinoline-3-carboxylate (700.0 mg, 2.21 mmol) in THF/water (5/5 mL) was added lithium hydroxide (53.0 mg, 2.21 mmol). The mixture was stirred at 0° C. for 1 h. The pH value of the mixture was adjusted to 7 with hydrochloric acid (1 mol/L). The mixture was evaporated in vacuo. The residue was purified by reverse phase flash column chromatography with 5-100% acetonitrile in water to afford the title compound (130.0 mg, 19%) as a yellow solid. MS (ESI) calculated for (C₁₆H₁₇NO₅) [M+H]⁺, 304.1; found, 304.2.

Step 3: Synthesis of tert-butyl 2-[[3-([3-[(1S)-1-[(4-methyl-4H-1,2,4-triazol-3-yl)sulfanyl]ethyl]phenyl]carbamoyl)isoquinolin-6-yl]oxy]acetate. Followed General procedure 2 with addition of reagents at 0° C. under N₂ to afford the title compound (210.0 mg, 72%). MS (ESI) calculated for (C₂₇H29N₅O₄S) [M+H]⁺, 520.2; found, 520.1.

Step 4: Synthesis of 2-[[3-([3-[(1S)-1-[(4-methyl-4H-1,2,4-triazol-3-yl)sulfanyl]ethyl]phenyl]carbamoyl)isoquinolin-6-yl]oxy]acetic acid. To a solution of the product from the previous step (260 mg, 0.50 mmol) in DCM (2 mL) was added trifluoroacetic acid (2 mL). The mixture was stirred at 0° C. for 1 h. The mixture was evaporated in vacuo. The residue was dissolved with water. The pH value of the mixture was adjusted to 6 with sodium bicarbonate (1 mol/L). The mixture was evaporated in vacuo. The residue was purified by Prep-HPLC to afford the title compound (160 mg, 62%) as a colorless solid. MS (ESI) calculated for (C₂₃H₂₁N₅O₄S) [M+H]⁺, 464.1; found, 464.2. ¹H NMR (400 MHz, DMSO-d₆) δ 13.20 (s, 1H), 10.74 (s, 1H), 9.33 (s, 1H), 8.57-8.55 (m, 2H), 8.24 (d, J=9.2 Hz, 1H), 8.00 (s, 1H), 7.90-7.88 (m, 1H), 7.64 (d, J=2.4 Hz, 1H), 7.52-7.49 (m, 1H), 7.33-7.29 (m, 1H), 7.04 (d, J=8.0 Hz, 1H), 4.93 (s, 2H), 4.71-4.66 (m, 1H), 3.40 (s, 3H), 1.68 (d, J=6.8 Hz, 3H).

### Example 306: (S)-N-(3-(1-(4-methyl-4H-1,2,4-triazol-3-ylthio)ethyl)phenyl)-6-(2-(methylamino)-2-oxoethoxy)isoquinoline-3-carboxamide (306a)

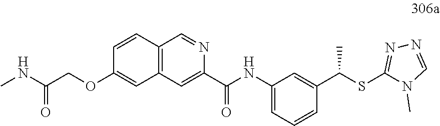 [SMILES: CNC(=O)COc1ccc2cnc(C(=O)Nc3cccc([C@H](C)Sc4nncn4C)c3)cc2c1]

Step 1: Synthesis of methyl 6-(2-(methylamino)-2-oxoethoxy)isoquinoline-3-carboxylate. To a solution of methyl 6-hydroxyisoquinoline-3-carboxylate (6.7 g, 33.00 mmol) in DMF (100 mL) was added 2-chloro-N-methylacetamide (4.3 g, 39.80 mmol) and potassium carbonate (6.8 g, 49.40 mmol). The mixture was stirred at 80° C. for 2 h. General Work-up Procedure 1 was followed and the resulting residue was purified by flash column chromatography with 0-100% EtOAc in petroleum ether to afford the title compound (2.6 g, 29%) as a yellow solid. MS (ESI) calculated for (C₁₄H₁₄N₂O₄) [M+H]⁺, 275.1; found, 274.9.

Step 2: Synthesis of 6-(2-(methylamino)-2-oxoethoxy)isoquinoline-3-carboxylic acid. To a solution of methyl 6-(2-(methylamino)-2-oxoethoxy)isoquinoline-3-carboxylate (2.6 g, 9.48 mmol) in THF (30 mL) and water (30 mL) was added lithium hydroxide (683.0 mg, 28.50 mmol). The mixture was stirred at rt for 4 h. The mixture was diluted with water. The pH value of the mixture was adjusted to 5 with hydrochloric acid (1 mol/L). The solid was collected by filtration and dried to afford the title compound (1.9 g, crude) as a yellow solid, which was used without purification. MS (ESI) calculated for (C₁₃H₂N₂O₄) [M+H]⁺, 261.1; found, 260.9.

Step 3: Synthesis of 306a. Followed General procedure 2 using product from the previous step (900.0 mg, crude) and A-a with stirring at 0° C. to afford the title compound (1.1 g, 64%). MS (ESI) calculated for (C₂₄H₂₄N₆O₃S) [M+H]⁺, 477.2; found, 477.0. H NMR (300 MHz, DMSO-d₆) δ 10.75 (s, 1H), 9.34 (s, 1H), 8.57 (d, J=2.7 Hz, 2H), 8.27-8.20 (m, 2H), 8.00 (s, 1H), 7.90-7.87 (m, 1H), 7.63 (d, J=2.4 Hz, 1H), 7.56-7.52 (m, 1H), 7.33-7.29 (m, 1H), 7.04 (d, J=7.8 Hz, 1H), 4.70-4.65 (m, 3H), 3.39 (s, 3H), 2.70 (d, J=4.2 Hz, 3H), 1.68 (d, J=6.9 Hz, 3H).

(R)-N-(3-(1-(4-methyl-4H-1,2,4-triazol-3-ylthio)ethyl)phenyl)-6-(2-(methylamino)-2-oxoethoxy)isoquinoline-3-carboxamide (306b)

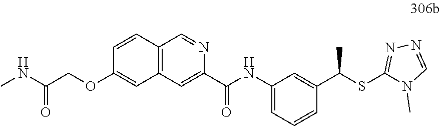 [SMILES: CNC(=O)COc1ccc2cnc(C(=O)Nc3cccc([C@@H](C)Sc4nncn4C)c3)cc2c1]

Followed General procedure 3 using 6-(2-(methylamino)-2-oxoethoxy)isoquinoline-3-carboxylic acid and A-b with stirring at 0° C. to afford the title compound (1.2 g, 63%). MS (ESI) calculated for (C₂₄H₂₄N₆O₃S) [M+H]⁺, 477.2; found, 477.0. H NMR (400 MHz, DMSO-d₆) δ 10.74 (s, 1H), 9.33 (s, 1H), 8.56 (d, J=3.6 Hz, 2H), 8.26-8.20 (m, 2H), 8.00 (s, 1H), 7.90-7.87 (m, 1H), 7.63 (d, J=2.4 Hz, 1H), 7.56-7.53 (m, 1H), 7.33-7.29 (m, 1H), 7.04 (d, J=8.0 Hz, 1H), 4.70-4.65 (m, 3H), 3.40 (s, 3H), 2.70 (d, J=4.8 Hz, 3H), 1.68 (d, J=7.2 Hz, 3H).

### Example 307: (S)-6-((1H-pyrazol-5-yl)methoxy)-N-(3-(1-((4-methyl-4H-1,2,4-triazol-3-yl)thio)ethyl)phenyl)isoquinoline-3-carboxamide (307)

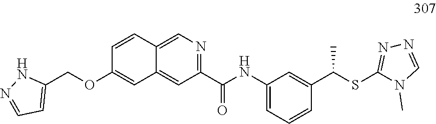 [SMILES: C[C@H](Sc1nncn1C)c1cccc(NC(=O)c2cc3cc(OCc4ccn[nH]4)ccc3cn2)c1]

Step 1: Synthesis of methyl 6-((1H-pyrazol-5-yl)methoxy)isoquinoline-3-carboxylate. To a solution of methyl 6-hydroxyisoquinoline-3-carboxylate (500.0 mg, 2.46 mmol) in acetonitrile (10 mL) was added 5-(chloromethyl)-1H-pyrazole hydrochloride (754.0 mg, 4.93 mmol) and cesium carbonate (2.41 g, 7.39 mmol). The solution was stirred at 80° C. for 4 h. General Work-up Procedure 1 was followed and the resulting residue was purified by reverse phase flash column chromatography with 5-100% acetonitrile in water to afford the title compound (160.0 mg, 23%) as a yellow solid. MS (ESI) calculated for (C₁₅H₁₃N₃O₃) [M+H]⁺, 284.1; found, 283.9.

Step 2: Synthesis of 6-((1H-pyrazol-5-yl)methoxy)isoquinoline-3-carboxylic acid. To a solution of methyl 6-((1H-pyrazol-5-yl)methoxy)isoquinoline-3-carboxylate (160.0 mg, 0.56 mmol) in THF/water (2/2 mL) was added lithium hydroxide (40.7 mg, 1.70 mmol). The mixture was stirred at rt for 4 h then concentrated. The mixture was diluted with water. The mixture was adjusted to pH 5 with hydrochloric acid (1 mol/L). The solid was collected by filtration and dried to afford the title compound (1.40 g, crude) as a yellow solid, which was used without purification. MS (ESI) calculated for (C₁₄H₁₁N₃O₃) [M+H]⁺, 270.1; found, 269.9.

Step 3: Synthesis of (S)-6-((1H-pyrazol-5-yl)methoxy)-N-(3-(1-(4-methyl-4H-1,2,4-triazol-3-ylthio)ethyl)phenyl)isoquinoline-3-carboxamide. Followed General procedure 2 using A-a and with stirring at 0° C. to afford the title compound (11.9 mg, 5%). MS (ESI) calculated for (C₂₅H₂₃N₇O₂S) [M+H]⁺, 486.2; found, 486.1. ¹H NMR (300 MHz, DMSO-d₆) δ 10.75 (s, 1H), 9.32 (s, 1H), 8.58 (s, 2H), 8.22 (d, J=9.0 Hz, 1H), 8.00 (s, 1H), 7.90 (d, J=8.1 Hz, 1H), 7.83 (d, J=2.1 Hz, 1H), 7.72 (s, 1H), 7.51-7.47 (m, 1H), 7.34-729 (m, 1H), 7.08-7.03 (m, 1H), 6.46 (d, J=2.4 Hz, 1H), 5.28 (s, 2H), 4.72-4.65 (m, 1H), 3.41 (s, 3H), 1.68 (d, J=6.9 Hz, 3H).

### Example 308: (S)-2-((3-((3-(1-((4-methyl-4H-1,2,4-triazol-3-yl)thio)ethyl)phenyl)carbamoyl)isoquinolin-7-yl)oxy)acetic acid (308)

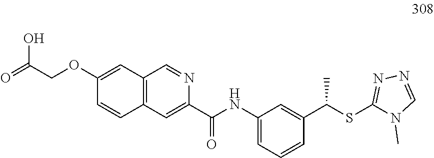 [SMILES: C[C@H](Sc1nncn1C)c1cccc(NC(=O)c2cc3ccc(OCC(=O)O)cc3cn2)c1]

Compound 308 (49.0 mg, 27%) was obtained according to procedures disclosed herein as a colorless solid. MS (ESI) calculated for (C₂₃H₂₁N₅O₄S) [M+H]⁺, 464.1; found, 464.2. ¹H NMR (400 MHz, DMSO-d₆) δ13.30 (s, 1H), 10.70 (s, 1H), 9.35 (s, 1H), 8.64 (s, 1H), 8.55 (s, 1H), 8.20 (d, J=9.2 Hz, 1H), 7.99 (s, 1H), 7.91-7.89 (m, 1H), 7.68 (d, J=2.4 Hz, 1H), 7.62-7.59 (m, 1H), 7.33-7.29 (m, 1H), 7.03 (d, J=7.6 Hz, 1H), 4.91 (s, 2H), 4.71-4.65 (m, 1H), 3.40 (s, 3H), 1.68 (d, J=6.8 Hz, 3H).

### Example 309: (S)-2-(2-(3-(1-(4-methyl-4H-1,2,4-triazol-3-ylthio)ethyl)phenylcarbamoyl)quinolin-5-yloxy)acetic acid (309)

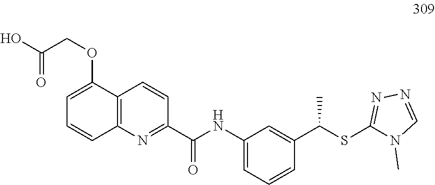 [SMILES: C[C@H](Sc1nncn1C)c1cccc(NC(=O)c2ccc3c(OCC(=O)O)cccc3n2)c1]

Compound 309 (21.7 mg, 24%) was obtained according to procedures disclosed herein as a colorless solid. MS (ESI) calc'd for (C₂₃H₂₁N₅O₄S) [M+H]⁺, 464.1; found, 463.9. ¹H NMR (300 MHz, DMSO-d₆) δ 13.21 (s, 1H), 10.76 (s, 1H), 8.83 (d, J=8.7 Hz, 1H), 8.55 (s, 1H), 8.27 (d, J=8.7 Hz, 1H), 7.99 (s, 1H), 7.95-7.79 (m, 3H), 7.34 (t, J=7.8 Hz, 1H), 7.15 (d, J=7.5 Hz, 1H), 7.06 (d, J=7.8 Hz, 1H), 5.00 (s, 2H), 4.70 (d, J=6.9 Hz, 1H), 3.41 (s, 3H), 1.70 (d, J=6.9 Hz, 3H).

### Example 310: (S)-N-(3-(1-((4-methyl-4H-1,2,4-triazol-3-yl)thio)ethyl)phenyl)-5-(2-(methylamino)-2-oxoethoxy)quinoline-2-carboxamide (310)

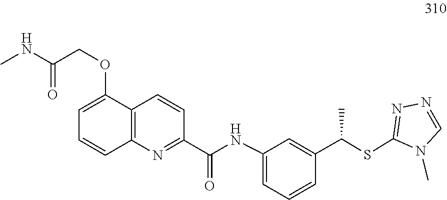 [SMILES: CNC(=O)COc1cccc2nc(C(=O)Nc3cccc([C@H](C)Sc4nncn4C)c3)ccc12]

To a solution of compound 309 (170 mg, 0.37 mmol), methanamine hydrochloride (37 mg, 0.55 mmol) in DMF (5 mL) were added N,N-diisopropylethylamine (189 mg, 1.46 mmol) and HATU (167 mg, 0.44 mmol) at 0° C. The solution was stirred at rt for 16 h, and then quenched by the addition of water (20 mL). Following General Work-up Procedure 1, the resulting crude product was purified by Prep-HPLC to afford compound 310 (27.7 mg, 16%) as a colorless solid. MS (ESI) calc'd for (C₂₄H₂₄N₆O₃S) [M+H]⁺, 477.2; found, 477.0. ¹H-NMR (300 MHz, DMSO-d₆) δ 10.77 (s, 1H), 9.10 (d, J=8.5 Hz, 1H), 8.55 (s, 1H), 8.26-8.20 (m, 2H), 8.00 (s, 1H), 7.95-7.77 (m, 3H), 7.34 (t, J=7.9 Hz, 1H), 7.16 (d, J=7.5 Hz, 1H), 7.06 (d, J=7.7 Hz, 1H), 4.76 (s, 2H), 4.67 (d, J=6.9 Hz, 1H), 3.41 (s, 3H), 2.74 (d, J=4.6 Hz, 3H), 1.70 (d, J=6.9 Hz, 3H).

### Example 311: 6-[2-(2-aminoethylamino)-2-oxo-ethoxy]-N-[3-[(1S)-1-[(4-methyl-1,2,4-triazol-3-yl)sulfanyl]ethyl]phenyl]isoquinoline-3-carboxamide hydrochloride (311)

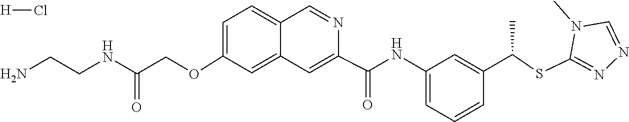 [SMILES: Cl ; C[C@H](Sc1nncn1C)c1cccc(NC(=O)c2cc3cc(OCC(=O)NCCN)ccc3cn2)c1]

Step 1: Synthesis of tert-butyl N-[2-[[2-[[3-[[3-[(1S)-1-[(4-methyl-1,2,4-triazol-3-yl)sulfanyl]ethyl]phenyl]carbamoyl]-6-isoquinolyl]oxy]acetyl]amino]ethyl]carbamate. To a solution of HATU (9.7 mg, 0.026 mmol) in 0.5 mL of DMF was added (S)-2-((3-((3-(1-((4-methyl-4H-1,2,4-triazol-3-yl)thio)ethyl)phenyl)=carbamoyl)=isoquinolin-6-yl)oxy)acetic acid (305, 10 mg, 0.022 mmol) and 2 drops of DIEA, stirred for 5 min at rt then added N-Boc-ethylenediamine (15 mg, 0.095 mmol) and stirred The mixture overnight at rt. The mixture was filtered and directly purified by HPLC followed by lyophilization in acetonitrile/water (1:1 v/v) afforded the title compound (6.9 mg, 44%) as a colorless solid after lyophilization. LCMS: C₃₀H₃₅N₇O₅S requires: 605.2, found: m/z=606.3 [M+H]⁺.

Step 2: Synthesis of 6-[2-(2-aminoethylamino)-2-oxo-ethoxy]-N-[3-[(1S)-1-[(4-methyl-1,2,4-triazol-3-yl)sulfanyl]ethyl]phenyl]isoquinoline-3-carboxamide hydrochloride. To a solution of tert-butyl (S)-(2-(2-((3-((3-(1-((4-methyl-4H-1,2,4-triazol-3-yl)thio)ethyl)phenyl)carbamoyl)isoquinolin-6-yl)oxy)acetamido)ethyl)carbamate (45 mg, 0.074 mmol) in THF (1 mL) was added HCl (1 mL, 4 N in dioxane) and methanol (1 mL). Stir at rt for 12 h. The resulting suspension was concentrated to give the title compound (40 mg, 100%) as an off-white solid. 1H NMR (500 MHz, Methanol-d₄) δ 9.53 (s, 1H), 9.46 (s, 1H), 9.01 (s, 1H), 8.46 (d, J=8.9 Hz, 1H), 7.96 (t, J=2.0 Hz, 1H), 7.87-7.77 (m, 2H), 7.75 (ddd, J=8.1, 2.2, 1.0 Hz, 1H), 7.41 (t, J=7.9 Hz, 1H), 7.26 (dt, J=7.6, 1.3 Hz, 1H), 4.97 (q, J=7.0 Hz, 1H), 4.93 (s, 2H), 3.70 (s, 3H), 3.62 (t, J=5.9 Hz, 2H), 3.15 (t, J=5.9 Hz, 2H), 1.87 (d, J=7.0 Hz, 3H). LCMS: m/z, 506.3 (M+1). LCMS: C₂₅H₂₇N₇O₃S requires: 505.2, found: m/z=506.3 [M+H]⁺.

### Example 312a and 312b: (S)-N-(3-(1-(5-methyl-1H-1,2,3-triazol-1-yl)propan-2-yl)phenyl)-6-(trifluoromethyl)picolinamide (312a) and (R)-N-(3-(1-(5-methyl-1H-1,2,3-triazol-1-yl)propan-2-yl)phenyl)-6-(trifluoromethyl)picolinamide (312b)

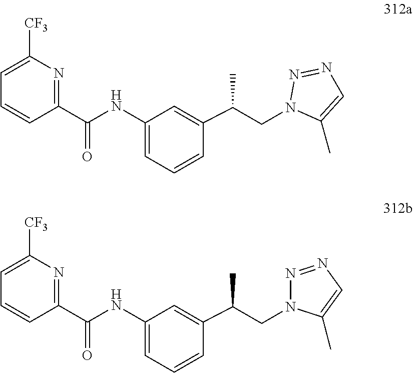 [SMILES: Cc1cnnn1C[C@@H](C)c1cccc(NC(=O)c2cccc(C(F)(F)F)n2)c1 ; Cc1cnnn1C[C@H](C)c1cccc(NC(=O)c2cccc(C(F)(F)F)n2)c1]

The enantiomers of the racemic mixture of Example 218 (54 mg) were separated using chiral chromatography on a IG column with CO₂ and methanol as mobile phase to afford (S)-N-(3-(1-(5-methyl-1H-1,2,3-triazol-1-yl)propan-2-yl)phenyl)-6-(trifluoromethyl)picolinamide (312a, 20 mg, 38%) and (R)-N-(3-(1-(5-methyl-1H-1,2,3-triazol-1-yl)propan-2-yl)phenyl)-6-(trifluoromethyl)picolinamide (312b, 16 mg, 30%) as off-white solids.

(S)-N-(3-(1-(5-methyl-1H-1,2,3-triazol-1-yl)propan-2-yl)phenyl)-6-(trifluoro-methyl)picolinamide (312a): ¹H NMR (500 MHz, Chloroform-d) δ 9.78 (s, 1H), 8.53 (d, J=8.0 Hz, 1H), 8.17 (td, J=7.9, 0.7 Hz, 1H), 7.92 (dd, J=7.7, 1.0 Hz, 1H), 7.74 (t, J=1.9 Hz, 1H), 7.65-7.55 (m, 1H), 7.43 (s, 1H), 7.32 (t, J=7.9 Hz, 1H), 6.87 (dt, J=7.6, 1.3 Hz, 1H), 4.51 (dd, J=13.7, 6.9 Hz, 1H), 4.33 (dd, J=13.7, 8.0 Hz, 1H), 3.52 (q, J=7.2 Hz, 1H), 2.01 (d, J=0.8 Hz, 3H), 1.45 (d, J=7.0 Hz, 3H). LCMS: C₁₉H₁₈F₃N₅O requires: 389.2, found: 390.4 [M+H]+

(R)-N-(3-(1-(5-methyl-1H-1,2,3-triazol-1-yl)propan-2-yl)phenyl)-6-(trifluoro-methyl)picolinamide (312b): ¹H NMR (500 MHz, Chloroform-d) δ 9.77 (s, 1H), 8.53 (d, J=1 & Hz, 1H), 8.17 (t, J=7.8 Hz, 1H), 7.92 (dd, J=7.8, 1.0 Hz, 1H), 7.73 (t, J=2.0 Hz, 1H), 7.60 (ddd, J=8.1, 2.2, 1.0 Hz, 1H), 7.41 (s, 1H), 7.32 (t, J=7.9 Hz, 1H), 6.87 (dt, J=7.6, 1.4 Hz, 1H), 4.50 (dd, J=3.1, 7.0 Hz, 1H), 4.32 (dd, J=13.7, 7.9 Hz, 1H), 3.52 (q, J=2 Hz, 1H), 2.00 (s, 3H), 1.44 (d, J=7.0 Hz, 3H). LCMS: C₁₉H₁₈F₃N₅O requires: 389.2, found: 390.4 [M+H]+

### Example 313: (R)-N-(3-(1-(4-Methyl-4H-1,2,4-triazol-3-yl)propan-2-yl)phenyl)-1,4,5,6-tetrahydrocyclopenta[c]pyrazole-3-carboxamide (313)

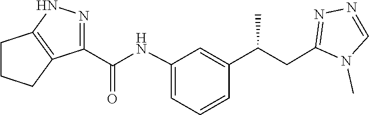 [SMILES: C[C@H](Cc1nncn1C)c1cccc(NC(=O)c2n[nH]c3c2CCC3)c1]

The amide bond formation reaction was carried out in a similar fashion as for 184 using 1,4,5,6-tetrahydrocyclopenta[c]pyrazole-3-carboxylic acid (77 mg, 0.51 mmol, 1.1 eq.) and D-a (100 mg, 0.46 mmol, 1 eq.) as reactants afforded the title compound (81 mg, 51%) as an off-white solid: ¹H NMR (500 MHz, DMSO-d₆) 12.95 (s, 1H), 9.72 (s, 1H), 8.28 (s, 1H), 7.75 (s, 1H), 7.65 (s, 1H), 7.21 (q, J=4.7 Hz, 1H), 6.94 (s, 1H), 3.21 (q, J=7.2 Hz, 1H), 2.95 (d, J=7.4 Hz, 2H), 2.71 (s, 4H), 2.57-2.41 (m, 2H), 1.27 (d, J=6.9 Hz, 3H); LCMS: C₁₉H₂₂N₆O requires: 351, found: m/z=352 [M+H]⁺.

### Example 314: (R)-N-(3-(1-(4-Methyl-4H-1,2,4-triazol-3-yl)propan-2-yl)phenyl)-3-(trifluoromethyl)-1H-pyrazole-5-carboxamide (314)

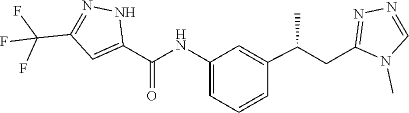 [SMILES: C[C@H](Cc1nncn1C)c1cccc(NC(=O)c2cc(C(F)(F)F)n[nH]2)c1]

The amide bond formation reaction was carried out in a similar fashion as for 184 using 3-(trifluoromethyl)-1H-pyrazole-5-carboxylic acid (61 mg, 0.34 mmol, 1.5 eq.) and D-a (50 mg, 0.23 mmol, 1 eq.) as reactants afforded the title compound (trifluoroacetic acid salt, 63 mg, 56%) as an off-white solid: 1H NMR (500 MHz, DMSO-d₆) 14.58 (d, J=32.8 Hz, 1H), 10.26 (s, 1H), 8.78 (s, 1H), 7.54 (s, 2H), 7.47 (s, 1H), 7.25 (t, J=7.8 Hz, 1H), 7.03-6.97 (m, 1H), 3.52 (s, 3H), 3.22 (h, J=6.8 Hz, 1H), 3.12-3.02 (m, 2H), 1.25 (d, J=6.9 Hz, 3H); LCMS: C₁₇H₁₇F₃N₆O requires: 378, found: m/z=379 [M+H]⁺.

### Example 315: (R)-N-(3-(1-(4-Methyl-4H-1,2,4-triazol-3-yl)propan-2-yl)phenyl)-2-(trifluoromethyl)oxazole-4-carboxamide (315)

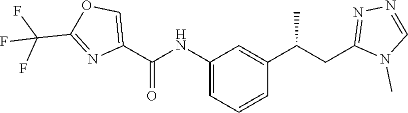 [SMILES: C[C@H](Cc1nncn1C)c1cccc(NC(=O)c2coc(C(F)(F)F)n2)c1]

The amide bond formation reaction was carried out in a similar fashion as for 184 using 2-(trifluoromethyl)oxazole-4-carboxylic acid (61 mg, 0.34 mmol, 1.5 eq.) and D-a (50 mg, 0.23 mmol, 1.0 eq.) as reactants afforded the title compound (trifluoroacetic acid salt, 29 mg, 26%) as an off-white solid: ¹H NMR (500 MHz, DMSO-d₆) δ 10.37 (s, 1H), 9.15 (s, 1H), 8.94 (s, 1H), 7.75-7.62 (m, 2H), 7.29 (t, J=7.8 Hz, 1H), 7.05 (dt, J=7.7, 1.3 Hz, 1H), 3.60 (s, 3H), 3.28 (h, J=6.8 Hz, 1H), 3.22-3.08 (m, 2H), 1.32 (d, J=6.8 Hz, 3H); LCMS: C₁₇H₁₆F₃N₅O₂ requires: 379, found: m/z=380 [M+H]⁺.

### Example 316: (R)-N-(3-(1-(4-Methyl-4H-1,2,4-triazol-3-yl)propan-2-yl)phenyl)pyrazolo[1,5-a]pyrimidine-5-carboxamide (316)

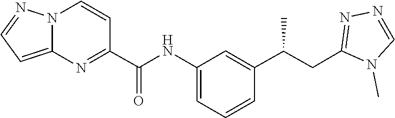 [SMILES: C[C@H](Cc1nncn1C)c1cccc(NC(=O)c2ccn3nccc3n2)c1]

The amide bond formation reaction was carried out in a similar fashion as for 184 using pyrazolo[1,5-a]pyrimidine-5-carboxylic acid (61 mg, 0.37 mmol, 1.6 eq.) and D-a (50 mg, 0.23 mmol, 1.0 eq.) as reactants afforded the title compound (trifluoroacetic acid salt, 52 mg, 48%) as an off-white solid: ¹H NMR (500 MHz, DMSO-d₆) δ 10.70 (s, 1H), 9.35 (dd, J=7.2, 1.0 Hz, 1H), 8.98 (s, 1H), 8.43 (d, J=2.4 Hz, 1H), 7.83 (t, J=1.9 Hz, 1H), 7.80 (ddd, J=8.1, 2.1, 1.0 Hz, 1H), 7.64 (d, J=7.2 Hz, 1H), 7.32 (t, J=7.8 Hz, 1H), 7.06 (dt, J=7.7, 1.3 Hz, 1H), 6.99 (dd, J=2.4, 0.9 Hz, 1H), 3.63 (s, 3H), 3.31 (h, J=6.8 Hz, 1H), 3.25-3.14 (m, 2H), 1.34 (d, J=6.9 Hz, 3H); LCMS: C₁₉H₁₉N₇O requires: 361, found: m/z=362 [M+H]⁺.

### Example 317: (R)-N-(3-(1-(4-Methyl-4H-1,2,4-triazol-3-yl)propan-2-yl)phenyl)pyrrolo[1,2-c]pyrimidine-3-carboxamide (317)

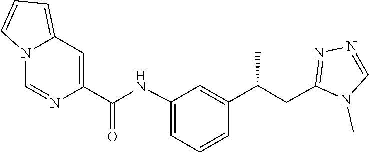 [SMILES: C[C@H](Cc1nncn1C)c1cccc(NC(=O)c2cc3cccn3cn2)c1]

The amide bond formation reaction was carried out in a similar fashion as for 184 using pyrazolo[1,5-a]pyrimidine-5-carboxylic acid (61 mg, 0.37 mmol, 1.6 eq.) and D-a (50 mg, 0.23 mmol, 1.0 eq.) as reactants afforded the title compound (trifluoroacetic acid salt, 56 mg, 51%) as an off-white solid: ¹H NMR (500 MHz, DMSO-d₆) δ 10.26 (s, 1H), 9.31 (t, J=1.2 Hz, 1H), 9.00 (s, 1H), 8.22 (d, J=1.4 Hz, 1H), 7.95-7.90 (m, 1H), 7.80 (tt, J=3.1, 1.5 Hz, 2H), 7.33-7.21 (m, 1H), 7.07 (dd, J=3.8, 2.8 Hz, 1H), 7.01 (dt, J=7.7, 1.4 Hz, 1H), 6.86 (dd, J=3.9, 1.1 Hz, 1H), 3.64 (s, 3H), 3.35-3.13 (m, 3H), 1.34 (d, J=6.8 Hz, 3H); LCMS: C₂₀H₂₀N₆O requires: 360, found: m/z=361 [M+H]⁺.

### Example 318: (R)-N-(3-(1-(4-Methyl-4H-1,2,4-triazol-3-yl)propan-2-yl)phenyl)imidazo[1,2-a]pyrazine-6-carboxamide (318)

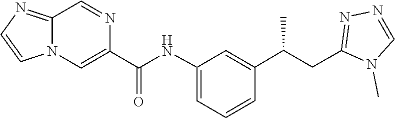 [SMILES: C[C@H](Cc1nncn1C)c1cccc(NC(=O)c2cn3ccnc3cn2)c1]

The amide bond formation reaction was carried out in a similar fashion as for 184 using imidazo[1,2-a]pyrazine-6-carboxylic acid (61 mg, 0.37 mmol, 1.6 eq.) and D-a (50 mg, 0.23 mmol, 1 eq.) as reactants afforded the title compound (trifluoroacetic acid salt, 70 mg, 69%) as an off-white solid: ¹H NMR (500 MHz, DMSO-d₆) δ 10.48 (s, 1H), 9.42 (d, J=1.5 Hz, 1H), 9.25-9.16 (m, 1H), 9.06 (s, 1H), 8.37 (s, 1H), 7.99 (d, J=1.1 Hz, 1H), 7.86-7.72 (m, 2H), 7.31 (t, J=7.9 Hz, 1H), 7.05 (dt, J=7.7, 1.3 Hz, 1H), 3.66 (s, 3H), 3.36-3.16 (m, 3H), 1.35 (d, J=6.8 Hz, 3H); LCMS: C₁₉H₁₉N₇O requires: 361, found: m/z=362 [M+H]⁺.

### Example 319: 4-cyclopropyl-N-[3-[(1R)-1-methyl-2-(4-methyl-1,2,4-triazol-3-yl)ethyl]phenyl]-1H-benzimidazole-2-carboxamide (319)

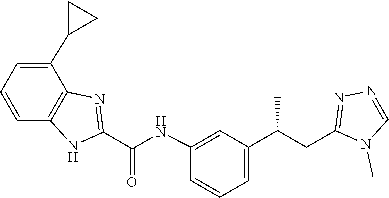 [SMILES: C[C@H](Cc1nncn1C)c1cccc(NC(=O)c2nc3c(C4CC4)cccc3[nH]2)c1]

Step 1. Synthesis of 4-bromo-2-(trichloromethyl)-1H-benzo[d]imidazole. Benzyl 2,2,2-trichloroacetimidate (2.2 mL, 11.7 mmol, 1.1 eq) was added over 2 min to a solution of 3-bromobenzene-1,2-diamine (2.0 g, 10.7 mmol, 1 eq) and acetic acid (20 mL) at 0° C. The cooling bath was removed and the solution maintained at rt for 2.5 h. The solution was added into ice-water mixture (200 mL) over 10 min with vigorous stirring and stirred for an additional 10 min. The resulting dark brown ppt was collected by vacuum filtration and dried to afford 3.3 g of the title compound.

Step 2. Synthesis of methyl 4-bromo-1H-benzo[d]imidazole-2-carboxylate. A solution of Na₂CO₃ (0.80 g, 7.5 mmol, 1 eq.) and water (7.3 mL) was added to a solution of 4-bromo-2-(trichloromethyl)-1H-benzo[d]imidazole (2.4 g, 7.5 mmol, 1.0 eq) and methanol (100 mL). The solution was heated at reflux for 24 h, then cooled to rt and the volume reduced by half under rotary evaporation. The solution was then added to well-stirred water (200 mL) over 10 min. The resulting black ppt was removed by filtration, and the solution extracted with EtOAc (3×30 mL). The combined organic phases were washed with brine (1×20 mL), dried and the residue purified by flash chromatography with EtOAc/hexanes to afford 1.56 g of the title compound as a tan solid.

Step 3. Synthesis of methyl 4-cyclopropyl-1H-benzo[d]imidazole-2-carboxylate. Cyclopropyl zinc bromide (4.0 mL of 0.5M solution in THF, 2.0 mmol, 2 eq) was added to a solution of methyl 4-bromo-1H-benzo[d]imidazole-2-carboxylate (0.25 g, 1.0 mmol, 1 eq), tetrakistriphenylphosphinepalladium(O) (110 mg, 0.1 mmol, 0.1 eq.) and THF (4 mL). The resulting black solution was heated at reflux for 4 h. The reaction was allowed to cool to rt, then poured into sat NH₄Cl (50 mL) and EtOAc (50 mL). The phases were separated and the aq. extracted with EtOAc (2×20 mL). The combined organic phases were dried, filtered, and concentrated onto Celite. The residue was purified by chromatography over SiO₂ with EtOAc/hexanes to afford an inseparable mixture of starting material and product. The mixture was dissolved in methanol (5 mL) and stirred with 10% Pd·C (30 mg) under 1 atm of H₂ for 3 h. The mixture was filtered through Celite, then purified by flash chromatography with EtOAc/hexanes to afford 24 mg of the title compound.

Step 4. Synthesis of 4-cyclopropyl-1H-benzo[d]imidazole-2-carboxylic acid. A mixture of 4-cyclopropyl-1H-benzo[d]imidazole-2-carboxylate (54 mg, 0.25 mmol, 1 eq), LiOH H₂O (32 mg, 0.75 mmol, 3 eq), THF (5 mL) and water (2 mL) was stirred vigorously for 24 h. The mixture was poured into 0.2N HCl (15 mL) and the resulting ppt was collected to afford 25 mg of the title compound as a colorless solid.

Step 5. The amide bond formation reaction was carried out in a similar fashion as for 74 using 4-cyclopropyl-1H-benzimidazole-2-carboxylic acid (24 mg, 0.12 mmol, 1.0 eq.) and D-a (28 mg, 0.13 mmol, 1.1 eq.) as reactants afford the title compound (39 mg) as an off-white solid: ¹H NMR (500 MHz, Acetonitrile-d₃) δ 9.65 (s, 1H), 8.51 (s, 1H), 7.73 (ddd, J=8.1, 2.2, 1.0 Hz, 1H), 7.65 (t, J=2.0 Hz, 1H), 7.46 (dd, J=8.1, 1.0 Hz, 1H), 7.34 (t, J=7.9 Hz, 1H), 7.32-7.22 (m, 1H), 7.06 (dt, J=7.7, 1.3 Hz, 1H), 6.91 (d, J=7.4 Hz, 1H), 3.54 (s, 3H), 3.43-3.33 (m, 1H), 3.33-3.20 (m, 2H), 2.55 (ddd, J=13.7, 8.6, 5.1 Hz, 1H), 1.42 (d, J=6.8 Hz, 3H), 1.17-1.06 (m, 2H), 1.03-0.93 (m, 2H); LCMS: C₂₃H₂₄N₆O requires: 400, found: m/z=401 [M+H]⁺.

### Example 320: 4-(2-hydroxypropan-2-yl)-N-[3-[(2R)-1-(4-methyl-4H-1,2,4-triazol-3-yl)propan-2-yl]phenyl]-6-(trifluoromethyl)pyridine-2-carboxamide (320)

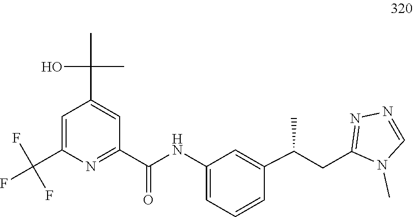 [SMILES: C[C@H](Cc1nncn1C)c1cccc(NC(=O)c2cc(C(C)(C)O)cc(C(F)(F)F)n2)c1]

Step 1: Synthesis of 2-[2-chloro-6-(trifluoromethyl)pyridin-4-yl]propan-2-ol. To a solution of 2-chloro-4-iodo-6-(trifluoromethyl)pyridine (4.3 g, 14.0 mmol) in THF (60 mL) was added i-PrMgCl (9.0 mL, 2 mol/L) at 0° C. under N₂. The mixture was stirred at 0° C. for 40 min. Then acetone (1.0 g, 17.2 mmol) was added to the above mixture and stirred at rt for another 2 h. The mixture was quenched by saturated aqueous NH₄Cl (100 mL) and the aqueous phase was extracted with EtOAc. The combined organic layers were washed with brine, dried, and filtered. The filtrate was evaporated under vacuum. The residue was purified by flash column chromatography with 20˜40% EtOAc in petroleum ether to afford the title compound (2.2 g, 65%) as a light yellow solid. MS (ESI) calculated for (C₉H₉ClF₃NO) [M+H]⁺, 240.0, found, 239.8.

Step 2: Synthesis of methyl 4-(2-hydroxypropan-2-yl)-6-(trifluoromethyl)pyridine-2-carboxylate. A mixture of 2-[2-chloro-6-(trifluoromethyl)pyridin-4-yl]propan-2-ol (988 mg, 4.12 mmol), Pd(dppf)Cl₂ (600.9 mg, 0.82 mmol) and TEA (3.0 mL, 21.58 mmol) in CH₃OH (20.0 mL) was heated at 70° C. for 16 h under CO (20 atm). The mixture was evaporated under vacuum. The residue was purified by flash column chromatography with 20-50% EtOAc in petroleum ether to afford the title compound (567 mg, 52%) as a light green solid. MS (ESI) calculated for (C₁₁H₁₂F₃NO₃) [M+H]⁺, 264.1, found, 263.8.

Step 3: Synthesis of 4-(2-hydroxypropan-2-yl)-6-(trifluoromethyl)pyridine-2-carboxylic acid. To a solution of methyl 4-(2-hydroxypropan-2-yl)-6-(trifluoromethyl)pyridine-2-carboxylate (567 mg, 2.15 mmol) in THF (8.0 mL) and water (4.0 mL) was added LiOH (200 mg, 8.35 mmol). The mixture was stirred at rt for 16 h. The pH value of the mixture was adjusted to 6-7 with HCl (1N). The mixture was evaporated under vacuum to afford the title compound (780 mg, crude) as an off-white solid, which was used without purification. MS (ESI) calculated for (C₁₀H₁₀F₃NO₃) [M+H]⁺, 250.1, found, 249.7.

Step 4: Synthesis of 4-(2-hydroxypropan-2-yl)-A-[3-[(2R)-1-(4-methyl-4H-1,2,4-triazol-3-yl)propan-2-yl]phenyl]-6-(trifluoromethyl)pyridine-2-carboxamide. The amide bond formation reaction was carried out in a similar fashion as for 184 using D-a (171.2 mg, 0.79 mmol) and 4-(2-hydroxypropan-2-yl)-6-(trifluoromethyl)pyridine-2-carboxylic acid (204.0 mg, 0.82 mmol) to afford the title compound (111 mg, 31%) as a light pink solid. MS (ESI) calculated for (C₂₂H₂₄F₃N₅O₂) [M+H]⁺, 448.2, found, 447.9. ¹H NMR (400 MHz, DMSO-d₆) δ 10.30 (s, 1H), 8.46 (d, J=1.2 Hz, 1H), 8.38 (s, 1H), 8.17 (d, J=1.2 Hz, 1H), 7.77-7.72 (m, 2H), 7.32-7.28 (m, 1H), 7.05 (d, J=7.6 Hz, 1H), 5.67 (s, 1H), 3.48 (s, 3H), 3.30-3.24 (m, 1H), 3.01 (d, J=7.2 Hz, 2H), 1.52 (s, 6H), 1.30 (d, J=7.2 Hz, 3H).

### Example 321: (R)-6-(Tert-butyl)-N-(3-(1-(4-methyl-4H-1,2,4-triazol-3-yl)propan-2-yl)phenyl)picolinamide (321)

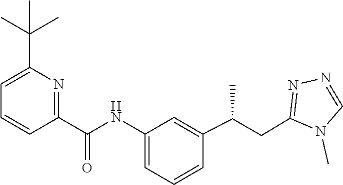 [SMILES: C[C@H](Cc1nncn1C)c1cccc(NC(=O)c2cccc(C(C)(C)C)n2)c1]

The amide bond formation reaction was carried out in a similar fashion as for 184 using 6-(tert-butyl)picolinic acid (46 mg, 0.25 mmol, 1.1 eq.) and (R)-3-(1-(4-methyl-4H-1,2,4-triazol-3-yl)propan-2-yl)aniline (50 mg, 0.23 mmol, 1.0 eq.) as reactants afforded the title compound (60 mg, 68%) as an off-white solid: ¹H NMR (500 MHz, DMSO-d₆) δ 10.19 (s, 1H), 8.41 (s, 1H), 8.04-7.94 (m, 2H), 7.77-7.67 (m, 3H), 7.36-7.28 (m, 1H), 7.06 (dt, J=7.8, 1.4 Hz, 1H), 3.49 (s, 3H), 3.32-3.25 (m, 1H), 3.04 (d, J=7.4 Hz, 2H), 1.43 (s, 9H), 1.37-1.30 (m, 3H); LCMS: C₂₂H₂₇N₅O requires: 377, found: m/z=378 [M+H]⁺.

### Example 322: (S)-4-Cyclopropyl-N-(3-(1-(4-methyl-4H-1,2,4-triazol-3-yl)propan-2-yl)phenyl)-6-(trifluoromethyl)picolinamide (322)

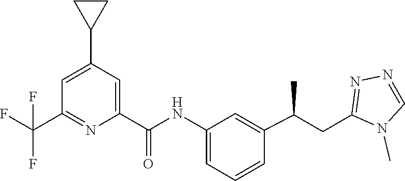 [SMILES: C[C@@H](Cc1nncn1C)c1cccc(NC(=O)c2cc(C3CC3)cc(C(F)(F)F)n2)c1]

Step 1: Synthesis of methyl 4-cyclopropyl-6-(trifluoromethyl)picolinate A mixture of methyl 4-chloro-6-(trifluoromethyl)picolinate (1.0 g, 4.2 mmol, 1 eq.), cyclopropylboronic acid pinacol ester (1.5 mL, 8.2 mmol, 2 eq.), [1,1′-Bis(diphenylphosphino)ferrocene]dichloropalladium(II), complex with dichloromethane (0.35 g, 0.42 mmol, 0.1 eq.) and potassium carbonate (1.8 g, 13 mmol, 3 eq.) in dioxane-water (10:1 v/v, 5.7 mL) was degassed and heated to 70° C. for 45 min. Water was added and extracted with EtOAc x2. The combined organic layers were washed with brine, dried (sodium sulfate), filtered and concentrated in vacuo. Purification on SiO₂ (ethyl acetate in hexanes) afforded the title compound as an off-white solid (0.78 g, 76% yield).

Step 2: Synthesis of (S)-4-cyclopropyl-N-(3-(1-(4-methyl-4H-1,2,4-triazol-3-yl)propan-2-yl)phenyl)-6-(trifluoromethyl)picolinamide. The trimethylaluminum amide bond formation reaction was carried out in a similar fashion as for 63 using methyl 4-cyclopropyl-6-(trifluoromethyl)picolinate (50 mg, 0.20 mmol, 1 eq.), and (S)-3-(1-(4-methyl-4H-1,2,4-triazol-3-yl)propan-2-yl)aniline (44 mg, 0.20 mmol, 1 eq.) as reactants afforded the title compound (84 mg, 96%) as an off-white solid: ¹H NMR (500 MHz, DMSO-d₆) δ 10.25 (s, 1H), 8.28 (s, 1H), 8.05 (d, J=1.6 Hz, 1H), 7.87 (d, J=1.6 Hz, 1H), 7.79-7.70 (m, 2H), 7.30 (t, J=7.9 Hz, 1H), 7.04 (dt, J=7.7, 1.3 Hz, 1H), 3.45 (s, 3H), 3.26 (q, J=7.1 Hz, 1H), 2.98 (d, J=7.3 Hz, 2H), 2.29 (tt, J=8.3, 4.9 Hz, 1H), 1.29 (d, J=6.9 Hz, 3H), 1.27-1.18 (m, 2H), 1.08-1.01 (m, 2H); LCMS: C₂₂H₂₂F₃N₅O requires: 429, found: m/z=430 [M+H]⁺.

### Example 323: 6-Methyl-N-(3-((R)-1-(4-methyl-4H-1,2,4-triazol-3-yl)propan-2-yl)phenyl)-2,4,5,6-tetrahydrocyclopenta[c]pyrazole-3-carboxamide (323)

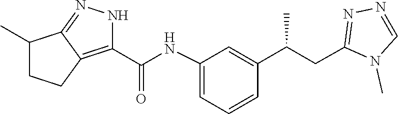 [SMILES: CC1CCc2c1n[nH]c2C(=O)Nc1cccc([C@H](C)Cc2nncn2C)c1]

The amide bond formation reaction was carried out in a similar fashion as for 184 using 6-methyl-1H, 4H, 5H, 6H-cyclopenta[c]pyrazole-3-carboxylic acid (42 mg, 0.25 mmol, 1.1 eq.) and 3-(2R)-1-(4-methyl-4H-1,2,4-triazol-3-yl)propan-2-yl aniline (50 mg, 0.23 mmol, 1 eq.) as reactants afforded the title compound (trifluoroacetic acid salt, 8.0 mg, 12%) as an off-white solid: 1H NMR (500 MHz, DMSO-d₆) δ 9.72 (s, 1H), 9.01 (s, 1H), 7.72 (s, 1H), 7.62 (dd, J=7.7, 2.0 Hz, 1H), 7.24 (t, J=7.9 Hz, 1H), 6.97 (dt, J=7.7, 1.3 Hz, 1H), 3.63 (s, 3H), 3.25 (tq, J=13.0, 7.1 Hz, 1H), 3.17 (ddd, J=14.2, 7.0, 4.3 Hz, 2H), 2.80-2.61 (m, 4H), 2.04 (ddt, J=13.7, 10.9, 6.2 Hz, 1H), 1.32 (d, J=6.8 Hz, 3H), 1.23 (d, J=6.9 Hz, 3H); LCMS: C₂₀H₂₄N₆O requires: 364, found: m/z=365 [M+H]⁺.

### Example 324: (R)-N-(3-(1-(4-Methyl-4H-1,2,4-triazol-3-yl)propan-2-yl)phenyl)pyrazolo[1,5-a]pyrimidine-3-carboxamide (324)

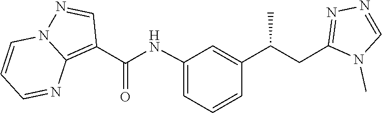 [SMILES: C[C@H](Cc1nncn1C)c1cccc(NC(=O)c2cnn3cccnc23)c1]

The amide bond formation reaction was carried out in a similar fashion as for 184 using pyrazolo[1,5-a]pyrimidine-3-carboxylic acid (42 mg, 0.25 mmol, 1.1 eq.) and 3-[(2R)-1-(4-methyl-4H-1,2,4-triazol-3-yl)propan-2-yl]aniline (50 mg, 0.23 mmol, 1 eq.) as reactants afforded the title compound (trifluoroacetic acid salt, 29 mg, 35%) as an off-white solid: ¹H NMR (500 MHz, DMSO-d₆) δ 9.92 (s, 1H), 9.40 (dd, J=7.0, 1.7 Hz, 1H), 8.93 (dd, J=4.3, 1.7 Hz, 1H), 8.72 (s, 1H), 7.69-7.53 (m, 2H), 7.39-7.21 (m, 3H), 7.03 (d, J=7.7 Hz, 1H), 3.56 (s, 3H), 3.30 (m, 1H), 3.11 (m, 2H), 1.33 (d, J=6.9 Hz, 3H); LCMS: C₁₋₉H₁₉N₇O requires: 361, found: m/z=362 [M+H]⁺.

### Example 325: (S)-2-Cyclopropyl-6-methyl-N-(3-(1-(4-methyl-4H-1,2,4-triazol-3-yl)propan-2-yl)phenyl)pyrimidine-4-carboxamide (325)

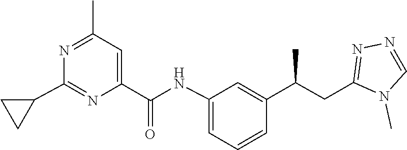 [SMILES: Cc1cc(C(=O)Nc2cccc([C@@H](C)Cc3nncn3C)c2)nc(C2CC2)n1]

The amide bond formation reaction was carried out in a similar fashion as for 184 using 2-cyclopropyl-6-methylpyrimidine-4-carboxylic acid (50 mg, 0.28 mmol, 1 eq.) and (S)-3-(1-(4-methyl-4H-1,2,4-triazol-3-yl)propan-2-yl)aniline (61 mg, 0.28 mmol, 1 eq.) as reactants afforded the title compound (46 mg, 44%) as an off-white solid: ¹H NMR (500 MHz, DMSO-d₆) δ 9.72 (s, 1H), 9.01 (s, 1H), 7.72 (s, 1H), 7.62 (dd, J=7.7, 2.0 Hz, 1H), 7.24 (t, J=7.9 Hz, 1H), 6.97 (dt, J=7.7, 1.3 Hz, 1H), 3.63 (s, 3H), 3.25 (tq, J=13.0, 7.1 Hz, 1H), 3.17 (ddd, J=14.2, 7.0, 4.3 Hz, 2H), 2.80-2.61 (m, 4H), 2.04 (ddt, J=13.7, 10.9, 6.2 Hz, 1H), 1.32 (d, J=6.8 Hz, 3H), 1.23 (d, J=6.9 Hz, 3H); LCMS: C₂₀H₂₄N₆O requires: 376, found: m/z=377 [M+H]⁺.

### Example 326: (R)-N-(3-(1-(4-methyl-4H-1,2,4-triazol-3-yl)propan-2-yl)phenyl)-4-(trifluoromethyl)-1H-imidazole-2-carboxamide (326)

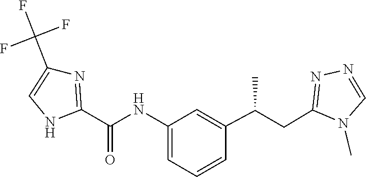 [SMILES: C[C@H](Cc1nncn1C)c1cccc(NC(=O)c2nc(C(F)(F)F)c[nH]2)c1]

Step 1: Synthesis of (R)-N-(3-(1-(4-methyl-4H-1,2,4-triazol-3-yl)propan-2-yl)phenyl)-4-(trifluoromethyl)-1H-imidazole-2-carboxamide. The amide bond formation reaction was carried out in a similar fashion as for 74 to afford the title compound (46.2 mg, 22%) as a colorless solid. MS (ESI) calculated for (C₁₇H₁₇F₃N₆O) [M+H]⁺, 379.1; found, 379.1. ¹H NMR (300 MHz, DMSO-d₆) δ 13.91 (s, 1H), 10.46 (s, 1H), 8.28 (s, 1H), 8.04 (s, 1H), 7.75-7.72 (m, 2H), 7.26 (t, J=7.8 Hz, 1H), 7.01 (d, J=7.8 Hz, 1H), 3.44 (s, 3H), 3.24-3.17 (m, 1H), 2.96 (d, J=12 Hz, 2H), 1.28 (d, J=6.9 Hz, 3H).

### Example 327: (R)-N-(3-(1-(4-Methyl-4H-1,2,4-triazol-3-yl)propan-2-yl)phenyl)-4,5,6,7-tetrahydro-2H-indazole-3-carboxamide (327)

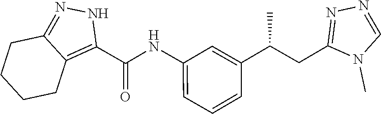 [SMILES: C[C@H](Cc1nncn1C)c1cccc(NC(=O)c2[nH]nc3c2CCCC3)c1]

The amide bond formation reaction was carried out in a similar fashion as for 184 using 4,5,6,7-tetrahydro-2H-indazole-3-carboxylic acid (46 mg, 0.28 mmol, 1.2 eq.) and 3-[(2R)-1-(4-methyl-4H-1,2,4-triazol-3-yl)propan-2-yl]aniline (50 mg, 0.23 mmol, 1 eq.) as reactants afforded the title compound (trifluoroacetic acid salt, 50 mg, 60%) as an off-white solid: ¹H NMR (500 MHz, DMSO-d₆) δ 9.74 (s, 1H), 9.08 (s, 1H), 7.74 (t, J=1.9 Hz, 1H), 7.70-7.62 (m, 1H), 7.23 (t, J=7.9 Hz, 1H), 6.96 (dt, J=7.6, 1.3 Hz, 1H), 3.65 (s, 3H), 3.31-3.22 (m, 1H), 3.18 (dt, J=15.0, 7.3 Hz, 2H), 2.69 (t, J=6.0 Hz, 2H), 2.67-2.60 (m, 2H), 1.79-1.65 (m, 4H), 1.32 (d, J=6.7 Hz, 3H); LCMS: C₂₀H₂₄N₆O requires: 364, found: m/z=365 [M+H]⁺.

### Example 328: (R)-N-(3-(1-(4-Methyl-4H-1,2,4-triazol-3-yl)propan-2-yl)phenyl)pyrazolo[1,5-a]pyrimidine-7-carboxamide (328)

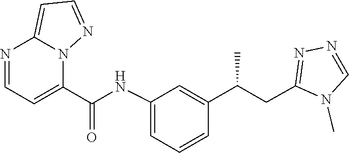 [SMILES: C[C@H](Cc1nncn1C)c1cccc(NC(=O)c2ccnc3ccnn23)c1]

The amide bond formation reaction was carried out in a similar fashion as for 184 using pyrazolo[1,5-a]pyrimidine-7-carboxylic acid (45 mg, 0.28 mmol, 1.2 eq.) and 3-[(2R)-1-(4-methyl-4H-1,2,4-triazol-3-yl)propan-2-yl]aniline (50 mg, 0.23 mmol, 1 eq.) as reactants afforded the title compound (trifluoroacetic acid salt, 77 mg, 92%) as an off-white solid: ¹H NMR (500 MHz, DMSO-d₆) δ 11.90 (s, 1H), 9.00 (s, 1H), 8.72 (d, J=4.2 Hz, 1H), 8.39 (d, J=2.5 Hz, 1H), 7.64-7.54 (m, 3H), 7.31 (t, J=7.9 Hz, 1H), 7.09 (dt, J=7.7, 1.3 Hz, 1H), 6.95 (d, J=2.5 Hz, 1H), 3.61 (s, 3H), 3.28 (p, J=7.1 Hz, 1H), 3.21-3.12 (m, 2H), 1.28 (d, J=6.8 Hz, 3H); LCMS: C₁₉H₁₉N₇O requires: 361, found: m/z=362 [M+H]⁺.

### Example 329: (R)-7-Methyl-N-(3-(1-(4-methyl-4H-1,2,4-triazol-3-yl)propan-2-yl)phenyl)pyrazolo[1,5-a]pyrimidine-5-carboxamide (329)

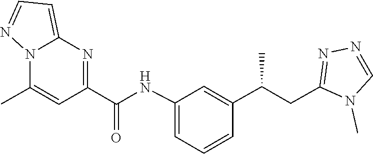 [SMILES: Cc1cc(C(=O)Nc2cccc([C@H](C)Cc3nncn3C)c2)nc2ccnn12]

The amide bond formation reaction was carried out in a similar fashion as for 184 using 7-methylpyrazolo[1,5-a]pyrimidine-5-carboxylic acid (49 mg, 0.28 mmol, 1.2 eq.) and 3-(2R)-1-(4-methyl-4H-1,2,4-triazol-3-yl)propan-2-yl aniline (50 mg, 0.23 mmol, 1 eq.) as reactants afforded the title compound (trifluoroacetic acid salt, 76 mg, 87%) as an off-white solid: ¹H NMR (500 MHz, DMSO-d₆) δ 10.67 (s, 1H), 9.12 (s, 1H), 8.44 (d, J=2.4 Hz, 1H), 7.85-7.79 (m, 2H), 7.67 (d, J=1.1 Hz, 1H), 7.36-7.28 (m, 1H), 7.06 (dt, J=7.6, 1.4 Hz, 1H), 6.99 (d, J=2.4 Hz, 1H), 3.67 (s, 3H), 3.30 (dd, J=15.2, 8.2 Hz, 1H), 3.23 (h, J=8.1 Hz, 2H), 2.87 (d, J=0.9 Hz, 3H), 1.35 (d, J=6.8 Hz, 3H); LCMS: C₂₀H₂₁N₇O requires: 375, found: m/z=376 [M+H]⁺.

### Example 330: (R)-2-cyclopropyl-6-methyl-N-(3-(1-(4-methylisoxazol-3-yl)propan-2-yl)phenyl)pyrimidine-4-carboxamide (330)

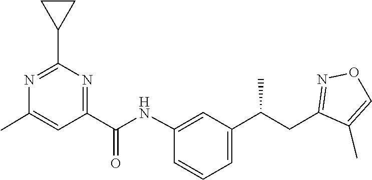 [SMILES: Cc1cc(C(=O)Nc2cccc([C@H](C)Cc3nocc3C)c2)nc(C2CC2)n1]

The amide bond formation reaction was carried out in a similar fashion as for 184 using 2-cyclopropyl-6-methyl-pyrimidine-4-carboxylic acid (82 mg, 0.46 mmol, 1.25 eq.) and 3-[(1R)-1-methyl-2-(4-methylisoxazol-3-yl)ethyl]aniline (80 mg, 0.37 mmol, 1 eq.) as reactants afford the title compound (54 mg) as an off-white solid: ¹H NMR (500 MHz, DMSO-d₆) δ 10.37 (s, 1H), 8.49 (d, J=1.2 Hz, 1H), 7.80-7.66 (m, 3H), 7.29 (t, J=7.7 Hz, 1H), 7.05 (dt, J=7.7, 1.3 Hz, 1H), 3.19-3.08 (m, 1H), 2.93-2.82 (m, 2H), 2.52 (s, 3H), 2.33 (td, J=8.1, 4.0 Hz, 1H), 1.90-1.82 (m, 3H), 1.26 (d, J=7.0 Hz, 3H), 1.18 (dt, J=4.7, 2.9 Hz, 2H), 1.14-1.07 (m, 2H); LCMS: C₂₂H₂₄N₄O₂ requires: 376, found: m/z=377 [M+H]⁺.

### Example 331: (R)-6-Cyclopropyl-5-methyl-N-(3-(1-(1-methyl-1H-imidazol-2-yl)propan-2-yl)phenyl)picolinamide (331a) and (S)-6-cyclopropyl-5-methyl-N-(3-(1-(1-methyl-1H-imidazol-2-yl)propan-2-yl)phenyl)picolinamide (331b)

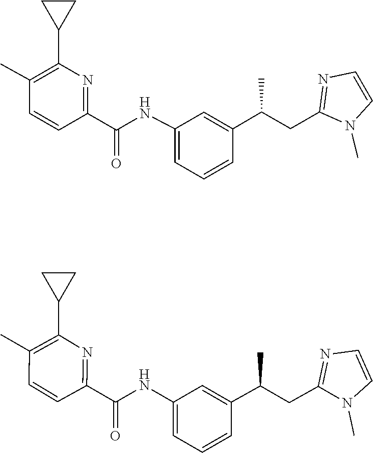 [SMILES: Cc1ccc(C(=O)Nc2cccc([C@H](C)Cc3nccn3C)c2)nc1C1CC1 ; Cc1ccc(C(=O)Nc2cccc([C@@H](C)Cc3nccn3C)c2)nc1C1CC1]

The amide bond formation reaction was carried out in a similar fashion as for 184. The enantiomers (89 mg) were separated using chiral chromatography on a CHIRALPAK IF column with CO₂ and methanol as mobile phase to afford:

(R)-6-Cyclopropyl-5-methyl-N-(3-(1-(1-methyl-1H-imidazol-2-yl)propan-2-yl)phenyl)picolinamide (25 mg, colorless solid): 1H NMR (500 MHz, DMSO-d₆) δ 9.89 (s, 1H), 7.76-7.65 (m, 2H), 7.63-7.57 (m, 1H), 7.54 (s, 1H), 7.20 (t, J=7.8 Hz, 1H), 6.95-6.91 (m, 1H), 6.89 (d, J=1.2 Hz, 1H), 6.71 (d, J=1.2 Hz, 1H), 3.35 (s, 3H), 3.23-3.13 (m, 1H), 2.82 (dd, J=7.3, 1.6 Hz, 2H), 2.42 (s, 3H), 2.19 (tt, J=8.1, 4.8 Hz, 1H), 1.23-1.13 (m, 5H), 0.96 (dq, J=8.1, 3.5 Hz, 2H); LCMS: C₂₃H₂₆N₄O requires: 374, found: m/z=375 [M+H]⁺.

(S)-6-cyclopropyl-5-methyl-N-(3-(1-(1-methyl-1H-imidazol-2-yl)propan-2-yl)phenyl)picolinamide (24 mg colorless solid): 1H NMR (500 MHz, DMSO-d₆) δ 9.97 (s, 1H), 7.83-7.73 (m, 2H), 7.67 (ddd, J=8.1, 2.2, 1.1 Hz, 1H), 7.61 (t, J=1.9 Hz, 1H), 7.28 (t, J=7.8 Hz, 1H), 7.01 (dt, J=7.6, 1.3 Hz, 1H), 6.94 (d, J=1.2 Hz, 1H), 6.75 (d, J=1.2 Hz, 1H), 3.42 (s, 3H), 3.24 (h, J=7.5, 7.0 Hz, 1H), 2.87 (dd, J=7.3, 2.1 Hz, 2H), 2.50 (s, 3H), 2.27 (tt, J=8.2, 4.8 Hz, 1H), 1.29-1.21 (m, 5H), 1.04 (dt, J=8.2, 3.2 Hz, 2H); LCMS: C₂₃H₂₆N₄O requires: 374, found: m/z=375 [M+H]⁺.

### Example 332: N-(3-((1S,2R)-2-(4-methyl-4H-1,2,4-triazol-3-yl)cyclopropyl)phenyl)-4-(trifluoromethyl)-1H-benzo[d]imidazole-2-carboxamide (332)

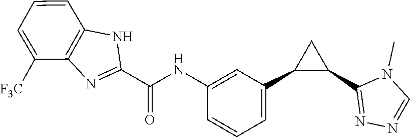 [SMILES: Cn1cnnc1[C@@H]1C[C@@H]1c1cccc(NC(=O)c2nc3c(C(F)(F)F)cccc3[nH]2)c1]

The amide bond formation reaction was carried out in a similar fashion as for 184 using 4-(trifluoromethyl)-1H-1,3-benzodiazole-2-carboxylic acid (59 mg, 0.26 mmol, 1.1 eq.) and 3-[(1S,2R)-2-(4-methyl-4H-1,2,4-triazol-3-yl)cyclopropyl]aniline (50 mg, 0.23 mmol, 1 eq.) as reactants afford the title compound (71 mg) as an off-white solid: ¹H NMR (500 MHz, DMSO-d₆) δ 13.91 (s, 1H), 10.38 (s, 1H), 8.14 (s, 1H), 7.87 (d, J=8.1 Hz, 1H), 7.73-7.60 (m, 3H), 7.50 (t, J=7.9 Hz, 1H), 7.09 (t, J=7.9 Hz, 1H), 6.68 (d, J=7.8 Hz, 1H), 3.44 (s, 3H), 2.65-2.60 (m, 2H), 1.89 (q, J=6.1 Hz, 1H), 1.56 (td, J=8.5, 4.9 Hz, 1H); LCMS: C₂₁H₁₇F₃N₆O requires: 426, found: m/z=427 [M+H]⁺.

### Example 333: 2-cyclopropyl-6-methyl-N-{3-[(1 S,2R)-2-(4-methyl-1,2,4-triazol-3-yl)cyclopropyl]phenyl}pyrimidine-4-carboxamide (333)

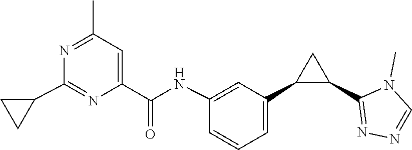 [SMILES: Cc1cc(C(=O)Nc2cccc([C@H]3C[C@H]3c3nncn3C)c2)nc(C2CC2)n1]

The amide bond formation reaction was carried out in a similar fashion as for 63 using methyl 2-cyclopropyl-6-methylpyrimidine-4-carboxylate (50 mg, 0.26 mmol, 1 eq.) and 3-[(1S,2R)-2-(4-methyl-4H-1,2,4-triazol-3-yl)cyclopropyl]aniline (111 mg, 0.23 mmol, 1 eq.) as reactants afford the TFA salt of the title compound (102 mg) as an off-white solid: ¹H NMR (500 MHz, DMSO-d₆) δ 10.33 (s, 1H), 8.84 (s, 1H), 7.69 (s, 1H), 7.64 (t, J=1.9 Hz, 1H), 7.57 (dd, J=8.0, 2.1 Hz, 1H), 7.17 (t, J=7.9 Hz, 1H), 6.80 (d, J=7.7 Hz, 1H), 2.86 (q, J=8.1 Hz, 1H), 2.77-2.66 (m, 1H), 2.35-2.27 (m, 1H), 1.98 (q, J=6.1 Hz, 1H), 1.77-1.65 (m, 1H), 1.24-1.13 (m, 2H), 1.13-0.99 (m, 2H); LCMS: C₂₁H₂₂N₆O requires: 374, found: m/z=375 [M+H]⁺.

### Example 334: (R)-N-(3-(1-(1,3,4-thiadiazol-2-yl)propan-2-yl)phenyl)-2-cyclopropyl-6-methylpyrimidine-4-carboxamide (334)

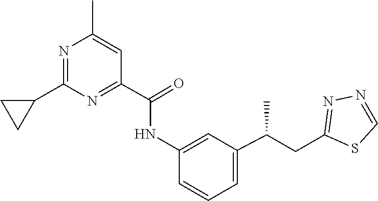 [SMILES: Cc1cc(C(=O)Nc2cccc([C@H](C)Cc3nncs3)c2)nc(C2CC2)n1]

The amide bond formation reaction was carried out in a similar fashion as for 63 using methyl 2-cyclopropyl-6-methylpyrimidine-4-carboxylate (50 mg, 0.26 mmol, 1 eq.) and 3-[(2R)-1-(1,3,4-thiadiazol-2-yl)propan-2-yl]aniline (98 mg, 0.45 mmol, 1 eq.) as reactants afford the TFA salt of the title compound (82 mg) as an off-white solid: ¹H NMR (500 MHz, DMSO-d₆) δ 10.37 (s, 1H), 9.40 (s, 1H), 7.80-7.67 (m, 3H), 7.30 (t, J=7.8 Hz, 1H), 7.08 (dt, J=7.6, 1.3 Hz, 1H), 3.45 (d, J=7.5 Hz, 2H), 3.28-3.16 (m, 1H), 2.52 (s, 3H), 2.33 (tt, J=8.1, 4.8 Hz, 1H), 1.30 (d, J=6.9 Hz, 3H), 1.18 (dt, J=4.6, 3.0 Hz, 2H), 1.13-1.06 (m, 2H); LCMS: C₂₀H₂₁N₅OS requires: 379, found: m/z=380 [M+H]⁺.

### Example 335: N-(3-{3-[(4-methyl-1,2,4-triazol-3-yl)methyl]oxetan-3-yl}phenyl)-4-(trifluoromethyl)-1H-1,3-benzodiazole-2-carboxamide (335)

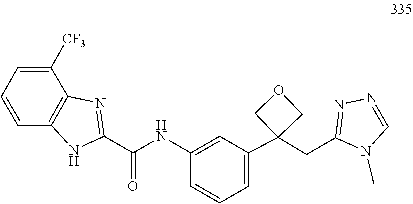 [SMILES: Cn1cnnc1CC1(c2cccc(NC(=O)c3nc4c(C(F)(F)F)cccc4[nH]3)c2)COC1]

The amide bond formation reaction was carried out in a similar fashion as for 74 using 4-(trifluoromethyl)-1H-1,3-benzodiazole-2-carboxylic acid (59 mg, 0.26 mmol, 1.1 eq.) and 3-(3-((4-methyl-4H-1,2,4-triazol-3-yl)methyl)oxetan-3-yl)aniline (50 mg, 0.23 mmol, 1 eq.) as reactants afford the title compound (46 mg) as an off-white solid: ¹H NMR (500 MHz, DMSO-d₆) δ 13.93 (s, 1H), 10.49 (s, 1H), 8.21 (s, 1H), 7.93-7.84 (m, 2H), 7.69 (d, J=7.5 Hz, 1H), 7.56-7.47 (m, 2H), 7.30 (t, J=7.9 Hz, 1H), 6.71 (d, J=7.7 Hz, 1H), 4.95 (d, J=6.0 Hz, 2H), 4.87 (d, J=6.0 Hz, 2H), 3.50 (s, 2H), 2.94 (s, 3H); LCMS: C₂₂H₁₉F₃N₆O₂ requires: 456, found: m/z=457 [M+H]⁺.

### Example 336: 2-cyclopropyl-6-methyl-N-(3-(3-((4-methyl-4H-1,2,4-triazol-3-yl)methyl)oxetan-3-yl)phenyl)pyrimidine-4-carboxamide (336)

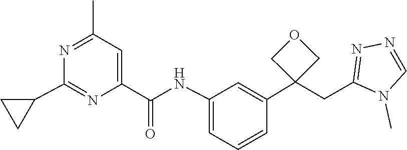 [SMILES: Cc1cc(C(=O)Nc2cccc(C3(Cc4nncn4C)COC3)c2)nc(C2CC2)n1]

The amide bond formation reaction was carried out in a similar fashion as for 74 using 3-{3-[(4-methyl-4H-1,2,4-triazol-3-yl)methyl]oxetan-3-yl}aniline (10.0 g, 40.9 mmol, 1.0 eq.) and 2-cyclopropyl-6-methylpyrimidine-4-carboxylic acid (7.3 g, 40.9 mmol, 1.0 eq.) to provide the title compound (13.8 g, 83%) as a colorless solid: 1H NMR (500 MHz, DMSO-d₆) δ 10.41 (s, 1H), 8.21 (s, 1H), 7.81 (ddd, J=8.2, 2.1, 1.0 Hz, 1H), 7.71 (s, 1H), 7.48 (t, J=2.0 Hz, 1H), 7.30 (t, J=7.9 Hz, 1H), 6.70 (dt, J=7.8, 1.2 Hz, 1H), 4.95 (d, J=6.0 Hz, 2H), 4.87 (d, J=6.0 Hz, 2H), 3.50 (s, 2H), 2.94 (s, 3H), 2.34 (tt, J=8.0, 4.7 Hz, 1H), 1.17 (dt, J=4.7, 2.9 Hz, 2H), 1.11 (ddd, J=10.3, 5.0, 2.9 Hz, 2H); LCMS: requires: 404, found: m/z=405 [M+H]+.

### Example 337: N-(3-(3-((4-methyl-4H-1,2,4-triazol-3-yl)methyl)oxetan-3-yl)phenyl)-2-(trifluoromethyl)pyrimidine-4-carboxamide (337)

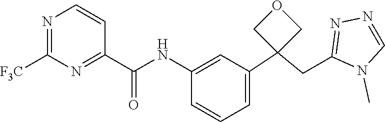 [SMILES: Cn1cnnc1CC1(c2cccc(NC(=O)c3ccnc(C(F)(F)F)n3)c2)COC1]

3-(3-((4-methyl-4H-1,2,4-triazol-3-yl)methyl)oxetan-3-yl)aniline (100 mg, 0.41 mmol, 1 eq) and 2-(trifluoromethyl)pyrimidine-4-carboxylic acid (85 mg, 0.45 mmol, 1.1 eq) were coupled in a similar fashion as for 74 to afford 136 mg of the title compound as an off-white solid: 1H NMR (500 MHz, DMSO-d₆) δ 10.64 (s, 1H), 9.36 (d, J=5.0 Hz, 1H), 8.75 (s, 1H), 8.36 (d, J=5.0 Hz, 1H), 7.83-7.73 (m, 1H), 7.50 (t, J=2.0 Hz, 1H), 7.34 (t, J=7.9 Hz, 1H), 6.81 (dt, J=7.7, 1.2 Hz, 1H), 4.92 (d, J=6.1 Hz, 2H), 4.88 (d, J=6.1 Hz, 2H), 3.63 (s, 2H), 3.07 (s, 3H); LCMS: C₁₉H₁₇F₃N₆O₂ requires: 418, found: m/z=419 [M+H]⁺.

### Example 338: 6-methyl-N-(3-(3-((4-methyl-4H-1,2,4-triazol-3-yl)methyl)oxetan-3-yl)phenyl)-2-(trifluoromethyl)pyrimidine-4-carboxamide (338)

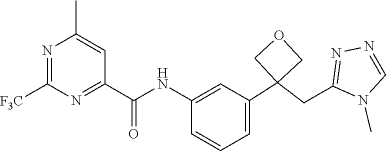 [SMILES: Cc1cc(C(=O)Nc2cccc(C3(Cc4nncn4C)COC3)c2)nc(C(F)(F)F)n1]

3-(3-((4-methyl-4H-1,2,4-triazol-3-yl)methyl)oxetan-3-yl)aniline (100 mg, 0.41 mmol, 1 eq) and 6-methyl-2-(trifluoromethyl)pyrimidine-4-carboxylic acid (93 mg, 0.45 mmol, 1.1 eq) were coupled in a similar fashion as for 74 to afford 136 mg of the title compound as an off-white solid: 1H NMR (500 MHz, DMSO-d₆) δ 10.57 (s, 1H), 8.68 (s, 1H), 8.26 (s, 1H), 7.80 (dd, J=8.2, 2.0 Hz, 1H), 7.48 (t, J=1.9 Hz, 1H), 7.33 (t, J=7.9 Hz, 1H), 6.80 (dt, J=7.7, 1.2 Hz, 1H), 4.92 (d, J=6.1 Hz, 2H), 4.87 (d, J=6.2 Hz, 2H), 3.61 (s, 2H), 3.05 (s, 3H), 2.73 (s, 3H); LCMS: C₂₀H₁₉F₃N₆O₂ requires: 432, found: m/z=433 [M+H]⁺.

### Example 339: 6-cyclopropyl-4-methyl-N-(3-(3-((4-methyl-4H-1,2,4-triazol-3-yl)methyl)oxetan-3-yl)phenyl)picolinamide (339)

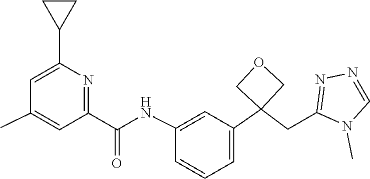 [SMILES: Cc1cc(C(=O)Nc2cccc(C3(Cc4nncn4C)COC3)c2)nc(C2CC2)c1]

Step 1. Synthesis of methyl 4-cyclopropyl-1H-benzo[d]imidazole-2-carboxylate. Cyclopropyl zinc bromide (16.2 mL of 0.5M solution in THF, 8.1 mmol, 1.5 eq) was added to a solution of methyl 2-chloro-6-methylpyrimidine-4-carboxylate (1.0 g, 5.4 mmol, 1 eq), tetrakistriphenylphosphinepalladium(O) (500 mg, 0.43 mmol, 0.08 eq) and THF (30 mL). The resulting black solution was heated at reflux for 24 h. The reaction was allowed to cool to rt, then poured into sat NH₄Cl (75 mL) and EtOAc (50 mL). The phases were separated and the aq extracted with EtOAc (2×10 mL). The combined organic phases were dried (sodium sulfate) filtered, and concentrated onto Celite. The residue was purified by chromatography over SiO₂ with EtOAc/hexanes to afford 510 mg of the title compound as a colorless solid.

Step 2. Synthesis of 2-cyclopropyl-6-methylpyrimidine-4-carboxylic acid. A mixture of methyl 2-cyclopropyl-6-methylpyrimidine-4-carboxylate (500 mg, 2.6 mmol, 1 eq), LiOH H₂O (330 mg, 7.8 mmol, 3 eq), THF (6 mL) and water (2 mL) was stirred vigorously for 17 h. The mixture was poured into 0.1 N HCl (50 mL) saturated with sodium sulfate. The product was extracted with 15% iPrOH/CHCl₃ (3×10 mL) and concentrated to afford 242 mg of the title compound as a colorless solid.

Step 3. Synthesis of 6-cyclopropyl-4-methyl-N-(3-(3-((4-methyl-4H-1,2,4-triazol-3-yl)methyl)oxetan-3-yl)phenyl)picolinamide. 3-(3-((4-methyl-4H-1,2,4-triazol-3-yl)methyl)oxetan-3-yl)aniline (50 mg, 0.2 mmol, 1 eq) and 6-cyclopropyl-4-methylpyridine-2-carboxylic acid (69 mg, 0.28 mmol, 1 eq) were coupled in a similar fashion as for 74 to afford 36 mg of the title compound as an off-white solid: 1H NMR (500 MHz, DMSO-d₆) δ 10.12 (s, 1H), 8.19 (s, 1H), 7.75 (ddd, J=8.2, 2.2, 1.0 Hz, 1H), 7.71 (d, J=1.4 Hz, 1H), 7.41 (t, J=1.9 Hz, 1H), 7.32 (t, J=1.2 Hz, 1H), 7.27 (t, J=7.9 Hz, 1H), 6.65 (dt, J=8.0, 1.1 Hz, 1H), 4.94 (d, J=6.0 Hz, 2H), 4.86 (d, J=6.0 Hz, 2H), 3.49 (s, 2H), 2.92 (s, 3H), 2.38 (s, 3H), 2.19 (tt, J=8.2, 4.8 Hz, 1H), 1.15-1.09 (m, 2H), 1.05-0.98 (m, 2H); LCMS: C₂₃H₂₅N₅O₂ requires: 403, found: m/z=404 [M+H]⁺.

### Example 340: 2-cyclobutyl-6-methyl-N-(3-(3-((4-methyl-4H-1,2,4-triazol-3-yl)methyl)oxetan-3-yl)phenyl)pyrimidine-4-carboxamide (340)

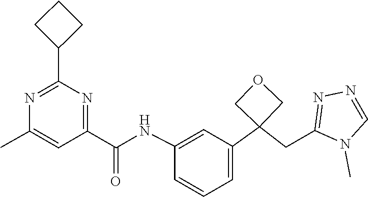 [SMILES: Cc1cc(C(=O)Nc2cccc(C3(Cc4nncn4C)COC3)c2)nc(C2CCC2)n1]

Step 1. Synthesis of methyl 2-cyclobutyl-6-methylpyrimidine-4-carboxylate. Synthesized according to the procedure of 339 Step 1 using cyclobutyl zinc bromide (16.1 mL of 0.5M solution in THF, 8.0 mmol, 1.5 eq) and methyl 2-chloro-6-methylpyrimidine-4-carboxylate (1.0 g, 5.4 mmol, 1 eq), to afford 510 mg of the title compound as a yellow solid.

Step 2. Synthesis of 2-cyclobutyl-6-methylpyrimidine-4-carboxylic acid. Methyl 2-cyclobutyl-6-methylpyrimidine-4-carboxylate (220 mg, 1.1 mmol, 1 eq) was hydrolyzed according to the procedure of 339 Step 2, to afford 177 mg the title compound as a yellow solid.

Step 3. Synthesis of 6-cyclopropyl-4-methyl-N-(3-(3-((4-methyl-4H-1,2,4-triazol-3-yl)methyl)oxetan-3-yl)phenyl)picolinamide. 3-(3-((4-methyl-4H-1,2,4-triazol-3-yl)methyl)oxetan-3-yl)aniline (63 mg, 0.26 mmol, 1 eq) 2-cyclobutyl-6-methylpyrimidine-4-carboxylic acid (50 mg, 0.26 mmol, 1 eq) were coupled in a similar fashion to 63 to afford 91 mg of the title compound as a yellow solid: 1H NMR (500 MHz, DMSO-d₆) δ 10.44 (s, 1H), 9.08 (s, 1H), 7.84-7.73 (m, 2H), 7.50 (t, J=1.9 Hz, 1H), 7.34 (t, J=7.9 Hz, 1H), 6.85 (dt, J=7.8, 1.3 Hz, 1H), 4.98-4.83 (m, 4H), 3.86 (pd, J=8.6, 1.1 Hz, 1H), 3.71 (s, 2H), 3.14 (s, 3H), 2.59 (s, 3H), 2.41-2.26 (m, 2H), 2.13-1.99 (m, 1H), 1.97-1.83 (m, 1H); LCMS: C₂₃H₂₆N₆O₂ requires: 418, found: m/z=419 [M+H]⁺.

### Example 341: 6-cyclopropyl-N-(3-(3-((4-methyl-4H-1,2,4-triazol-3-yl)methyl)oxetan-3-yl)phenyl)-2-(trifluoromethyl)pyrimidine-4-carboxamide (341)

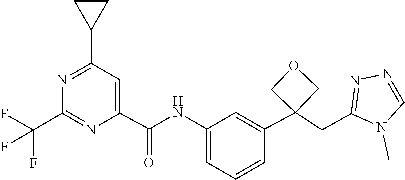 [SMILES: Cn1cnnc1CC1(c2cccc(NC(=O)c3cc(C4CC4)nc(C(F)(F)F)n3)c2)COC1]

The amide bond formation reaction was carried out in a similar fashion as for 74 using 6-cyclopropyl-2-(trifluoromethyl)pyrimidine-4-carboxylic acid (47 mg, 0.20 mmol, 1.0 eq.) and 3-(3-((4-methyl-4H-1,2,4-triazol-3-yl)methyl)oxetan-3-yl)aniline (50 mg, 0.20 mmol, 1.0 eq.) as reactants to afford the title compound (65 mg, 70%) as a colorless solid: ¹H NMR (500 MHz, DMSO-d6) δ 10.49 (s, 1H), 8.26 (s, 1H), 8.19 (s, 1H), 7.78 (ddd, J=8.2, 2.1, 1.0 Hz, 1H), 7.45 (t, J=2.0 Hz, 1H), 7.29 (t, J=7.9 Hz, 1H), 6.75-6.67 (m, 1H), 4.93 (d, J=5.9 Hz, 2H), 4.84 (d, J=6.0 Hz, 2H), 3.48 (s, 2H), 2.92 (s, 3H), 1.28 (dt, J=7.9, 3.4 Hz, 2H), 1.17 (dt, J=4.4, 3.2 Hz, 2H); LCMS: requires: 458, found: m/z=459 [M+H]⁺.

### Example 342: 2-cyclopropyl-6-methoxy-N-(3-(3-((4-methyl-4H-1,2,4-triazol-3-yl)methyl)oxetan-3-yl)phenyl)pyrimidine-4-carboxamide (342)

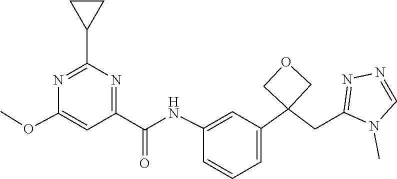 [SMILES: COc1cc(C(=O)Nc2cccc(C3(Cc4nncn4C)COC3)c2)nc(C2CC2)n1]

Step 1. Synthesis of methyl 2-cyclopropyl-6-methoxypyrimidine-4-carboxylate. methyl 2-chloro-6-methoxypyrimidine-4-carboxylate (960 mg, 4.7 mmol, 1 eq) and coupled with cyclopropyl zinc bromide as in 339 Step 1 to afford 402 mg of the title compounds as an off-white solid.

Step 2. Synthesis of 2-cyclopropyl-6-methoxypyrimidine-4-carboxylic acid. Methyl 2-cyclopropyl-6-methoxypyrimidine-4-carboxylate (400 mg, 1 mmol, 1 eq) was hydrolyzed according to the procedure of 339 Step 2, to afford 192 mg the title compound as a yellow solid.

Step 3. Synthesis of 2-cyclopropyl-6-methoxy-N-(3-(3-((4-methyl-4H-1,2,4-triazol-3-yl)methyl)oxetan-3-yl)phenyl)pyrimidine-4-carboxamide. 3-(3-((4-methyl-4H-1,2,4-triazol-3-yl)methyl)oxetan-3-yl)aniline (63 mg, 0.26 mmol, 1 eq) and 2-cyclopropyl-6-methoxypyrimidine-4-carboxylic acid (50 mg, 0.26 mmol, 1 eq) were coupled in a similar fashion as for 74 to afford 65 mg of the title compound as a colorless solid: ¹H NMR (500 MHz, DMSO-d₆) δ 10.36 (s, 1H), 8.20 (s, 1H), 7.78 (ddd, J=8.1, 2.1, 1.0 Hz, 1H), 7.48 (t, J=2.0 Hz, 1H), 7.28 (t, J=7.9 Hz, 1H), 7.17 (s, 1H), 6.68 (ddd, J=7.7, 1.8, 1.0 Hz, 1H), 4.93 (d, J=5.9 Hz, 2H), 4.85 (d, J=6.0 Hz, 2H), 3.95 (s, 3H), 3.48 (s, 2H), 2.92 (s, 3H), 2.29 (tt, J=8.0, 4.7 Hz, 1H), 1.23-1.15 (m, 2H), 1.15-1.06 (m, 2H); LCMS: C₂₂H₂₄N₆O₃ requires: 420, found: m/z=421 [M+H]⁺.

### Example 343: N-(3-(3-((4-Methyl-4H-1,2,4-triazol-3-yl)methyl)oxetan-3-yl)phenyl)-2,5,6,7-tetrahydropyrano[3,2-c]pyrazole-3-carboxamide (343)

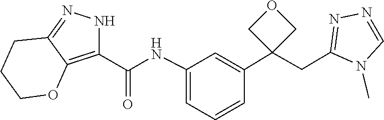 [SMILES: Cn1cnnc1CC1(c2cccc(NC(=O)c3[nH]nc4c3OCCC4)c2)COC1]

Step 1: Synthesis of 1,5,6,7-tetrahydropyrano[3,2-c]pyrazole-3-carboxylic acid A solution of ethyl 1,5,6,7-tetrahydropyrano[3,2-c]pyrazole-3-carboxylate (50 mg, 0.25 mmol, 1 eq.) in THF (0.89 mL) was treated with an aqueous solution of lithium hydroxide (1M, 0.96 mL, 0.96 mmol, 3.75 eq.) at rt. The mixture was stirred for 2 h at rt and quenched with hydrochloric acid (2 M, 0.48 mL, 0.96 mmol, 3.75 eq.). THF was removed in vacuo and the mixture was dried on a lyophilizer to yield the crude title compound.

Step 2: Synthesis of 2-cyclopropyl-N-(3-(3-((4-methyl-4H-1,2,4-triazol-3-yl)methyl)oxetan-3-yl)phenyl)-6-(4-methylpiperazin-1-yl)pyrimidine-4-carboxamide The amide bond formation reaction was carried out in a similar fashion as for 184 using the crude 1,5,6,7-tetrahydropyrano[3,2-c]pyrazole-3-carboxylic acid, (0.16 mmol, 1 eq.) and 3-(3-((4-methyl-4H-1,2,4-triazol-3-yl)methyl)oxetan-3-yl)aniline (50 mg, 0.16 mmol, 1 eq.) as reactants afforded the title compound (12.5 mg, 16%) as an off-white solid: ¹H NMR (500 MHz, DMSO-d₆) δ 12.87 (s, 1H), 8.12 (s, 1H), 7.58 (d, J=8.1 Hz, 1H), 7.24 (s, 1H), 7.14 (t, J=7.9 Hz, 1H), 6.51 (d, J=7.7 Hz, 1H), 4.84 (d, J=5.9 Hz, 2H), 4.76 (d, J=6.1 Hz, 2H), 4.08 (s, 2H), 3.39 (s, 3H), 2.83 (s, 2H), 2.64 (t, J=6.4 Hz, 2H), 1.88 (s, 2H); LCMS: C₂₀H₂₂N₆O₃ requires: 394, found: m/z=395 [M+H]⁺.

### Example 344: (R)-2-Cyclopropyl-6-methyl-N-(3-(1,1,2-trifluoro-1-(4-methyl-4H-1,2,4-triazol-3-yl)propan-2-yl)phenyl)pyrimidine-4-carboxamide (344)

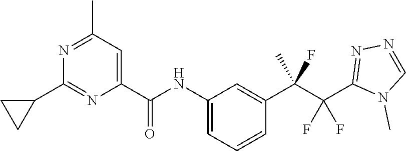 [SMILES: Cc1cc(C(=O)Nc2cccc([C@@](C)(F)C(F)(F)c3nncn3C)c2)nc(C2CC2)n1]

The amide bond formation reaction was carried out in a similar fashion as for 184 using 2-cyclopropyl-6-methylpyrimidine-4-carboxylic acid (98 mg, 0.44 mmol, 1.2 eq.) and (R)-3-(1,1,2-trifluoro-1-(4-methyl-4H-1,2,4-triazol-3-yl)propan-2-yl)aniline (100 mg, 0.37 mmol, 1 eq.) as reactants to afford the title compound (110 mg, 69%) as an off-white solid: ¹H NMR (500 MHz, DMSO-d₆) δ 10.59 (s, 1H), 8.64 (s, 1H), 8.02-7.94 (m, 2H), 7.73 (s, 1H), 7.45 (t, J=7.9 Hz, 1H), 7.13 (d, J=7.9 Hz, 1H), 3.46 (s, 3H), 2.53 (s, 3H), 2.39-2.31 (m, 1H), 1.97 (d, J=24.3 Hz, 3H), 1.27-1.18 (m, 2H), 1.12 (dt, J=8.2, 3.1 Hz, 2H); LCMS: C₂₁H₂₁F₃N₆O requires: 430, found: m/z=431 [M+H]⁺.

### Example 345: (R)-N-(3-(1,1,2-trifluoro-1-(4-methyl-4H-1,2,4-triazol-3-yl)propan-2-yl)phenyl)-4-(trifluoromethyl)-1H-benzo[d]imidazole-2-carboxamide (345)

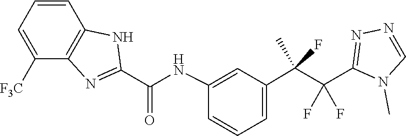 [SMILES: Cn1cnnc1C(F)(F)[C@](C)(F)c1cccc(NC(=O)c2nc3c(C(F)(F)F)cccc3[nH]2)c1]

(R)-3-(1,1,2-trifluoro-1-(4-methyl-4H-1,2,4-triazol-3-yl)propan-2-yl)aniline (65 mg, 0.24 mmol, 1 eq) and 4-(trifluoromethyl)-1H-benzo[d]imidazole-2-carboxylic acid (61 mg, 0.26 mmol, 1.1 eq) were coupled in a similar fashion as for 74 to afford 36 mg of the title compound as an off-white solid: ¹H NMR (500 MHz, DMSO-d₆) δ 13.94 (s, 1H), 10.73 (s, 1H), 8.63 (s, 1H), 8.15-7.96 (m, 2H), 7.89 (s, 1H), 7.68 (d, J=7.3 Hz, 1H), 7.51 (t, J=7.8 Hz, 1H), 7.45 (t, J=8.0 Hz, 1H), 7.13 (d, J=7.9 Hz, 1H), 3.44 (s, 3H), 1.97 (d, J=24.1 Hz, 3H); LCMS: C₂₁H₁₆F₆N₆O requires: 482, found: m/z=483 [M+H]⁺.

### Example 346: 2-cyclopropyl-N-(3-{1-[difluoro(4-methyl-4H-1,2,4-triazol-3-yl)methyl]cyclopropyl}phenyl)-6-methylpyrimidine-4-carboxamide (346)

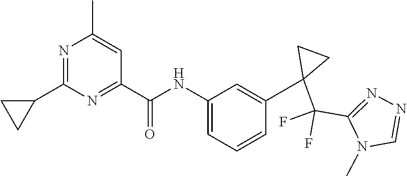 [SMILES: Cc1cc(C(=O)Nc2cccc(C3(C(F)(F)c4nncn4C)CC3)c2)nc(C2CC2)n1]

Step 1: Synthesis of ethyl 3-(3-((tert-butoxycarbonyl)amino)phenyl)-2,2-difluoro-3-hydroxypropanoate. To a mixture of ethyl 2-bromo-2,2-difluoroacetate (181.4 g, 893.6 mmol, 114.8 mL, 1.0 eq.), Zn (210.3 g, 3.22 mol, 3.6 eq.) and THF (1200 mL) was added DIBAL-H (1 M, 35.7 mL, 0.04 eq.) at 10-15° C. Then the mixture was stirred at 30° C. for 1 hr. A solution of ethyl 2-bromo-2,2-difluoroacetate (18.1 g, 89.3 mmol, 11.4 mL, 1.0 eq.), tert-butyl (3-formylphenyl)carbamate (WO2017072196) (208.0 g, 893.6 mmol, 1.0 eq.) and THF (300 mL) was added drop-wise to the mixture at 30° C. After the addition, the mixture was stirred at 30-50° C. for 6 hours. The mixture was quenched with saturated aqueous ammonium chloride and extracted with EtOAc. The combined organic phases were washed with brine, dried (sodium sulfate), and concentrated. The residue was purified by silica gel chromatography eluting with petroleum ether/ethyl acetate to give the title compound (545.0 g, 1.58 mol, 88%) as a yellow oil.

Step 2: Synthesis of ethyl 3-(3-((tert-butoxycarbonyl)amino)phenyl)-2,2-difluoro-3-oxopropanoate. To a solution of ethyl 3-(3-((tert-butoxycarbonyl)amino)phenyl)-2,2-difluoro-3-hydroxypropanoate (150 g, 434.3 mmol, 1.0 eq.) in dichloromethane (1500 mL) was added Dess-Martin periodinane (368.4 g, 868.7 mmol, 2.0 eq.) at 20-25° C. Then the mixture was stirred at 20-25° C. for 2 hours, Then quenched with saturated aqueous Na₂CO₃ to make pH=8-9 and then extracted with dichloromethane. The combined organic phase was washed with saturated aqueous sodium sulfite. The organic phase was concentrated. The residue was purified by silica gel chromatography eluting with petroleum ether/ethyl acetate to the title compound (120 g, 349.5 mmol, 80%) as a yellow oil.

Step 3: Synthesis of ethyl 3-(3-((tert-butoxycarbonyl)amino)phenyl)-2,2-difluorobut-3-enoate. To a solution of Ph₃PMeBr (62.4 g, 174.7 mmol, 1.2 eq.) in THF (750 mL) was added t-BuOK (19.6 g, 174.7 mmol, 1.2 eq.) at 0° C. Then the mixture was stirred at 0° C. for 2 hours. A solution of ethyl 3-(3-((tert-butoxycarbonyl)amino)phenyl)-2,2-difluoro-3-oxopropanoate (50.0 g, 145.6 mmol, 1.0 eq.) in THF (200 mL) was added drop-wise to the mixture at 0° C. After the addition, the mixture was stirred at 10-15° C. for 6 hours. The mixture was diluted with ice water and extracted with EtOAc. The organic phase was washed with brine, dried (sodium sulfate), and concentrated. The residue was purified by silica gel chromatography, eluting with petroleum ether/ethyl acetate to afford the title compound (300.0 g, 812.6 mmol, 70%) as a light yellow oil.

Step 4: Synthesis of ethyl 2-(3-(3-((tert-butoxycarbonyl)amino)phenyl)-4,5-dihydro-3H-pyrazol-3-yl)-2,2-difluoroacetate. To a solution of ethyl 3-(3-((tert-butoxycarbonyl)amino)phenyl)-2,2-difluorobut-3-enoate (57.0 g, 164.7 mmol, 1.0 eq.) and TBAF (1 M, 14.2 mL, 0.09 eq) in dichloromethane (100 mL) was added trimethylsilyldiazomethane (2 M, 142.5 mL, 1.73 eq.) at 0° C. After the addition, the mixture was stirred at 10° C. for 16 hours. Trimethylsilyldiazomethane (2 M, 57.0 mL) and TBAF (1 M, 14.2 mL) was added to the mixture at 0° C. The mixture was stirred at 10-15° C. for 3 hours. The mixture was quenched with saturated aqueous citric acid and extracted with dichloromethane. The organic phase was dried, concentrated. The residue was purified by silica gel chromatography eluting with petroleum ether/ethyl acetate to give the title compound (70.0 g, 182.5 mmol, 37%) as a light yellow oil.

Step 5: Synthesis of ethyl 2-(1-(3-((tert-butoxycarbonyl)amino)phenyl)cyclopropyl)-2,2-difluoroacetate. A solution of ethyl 2-(3-(3-((tert-butoxycarbonyl)amino)phenyl)-4,5-dihydro-3H-pyrazol-3-yl)-2,2-difluoroacetate (70.0 g, 182.5 mmol, 1.0 eq.) in o-xylene (300 mL) was stirred at 140-150° C. for 16 hours. The mixture was concentrated to give the title compound as a yellow oil which was used to next step without purification.

Step 6: Synthesis of tert-butyl (3-(l-(1,1-difluoro-2-hydrazinyl-2-oxoethyl)cyclopropyl)phenyl)carbamate. To a solution of ethyl 2-(1-(3-((tert-butoxycarbonyl)amino)phenyl)cyclopropyl)-2,2-difluoroacetate (64.9 g, 182.6 mmol, 1.0 eq.) in THF (1000 mL) was added hydrazine hydrate (46.6 g, 912.9 mmol, 45 mL, 98% purity, 5 eq.) at 10-15° C. Then the mixture was stirred at 20-25° C. for 16 hours. The mixture was diluted with EtOAc and then washed with water. The organic phase was dried, concentrated to give the title compound as a yellow oil, which was used to next step without purification.

Step 7: Synthesis of tert-butyl (3-(1-(1,1-difluoro-2-(2-(methylcarbamothioyl)hydrazinyl)-2-oxoethyl)cyclopropyl)phenyl)carbamate. To a solution of tert-butyl (3-(l-(1,1-difluoro-2-hydrazinyl-2-oxoethyl)cyclopropyl)phenyl)carbamate (62.3 g, 182.6 mmol, 1.0 eq.) in THF (500 mL) was added methylimino(thioxo)methane (26.7 g, 365.2 mmol, 24.96 mL, 2.0 eq.) at 20-25° C. Then the mixture was stirred at 70° C. for 1 hr. The mixture was diluted with EtOAc and then washed with water. The organic phase was dried, and concentrated to give the title compound as a yellow oil.

Step 8: Synthesis of tert-butyl (3-(1-(difluoro(5-mercapto-4-methyl-4H-1,2,4-triazol-3-yl)methyl)cyclopropyl)phenyl)carbamate. To a mixture of tert-butyl (3-(l-(1,1-difluoro-2-(2-(methylcarbamothioyl)hydrazinyl)-2-oxoethyl)cyclopropyl)phenyl)carbamate (75.6 g, 182.6 mmol, 1.0 eq.) in water (500 mL) was added NaOH (29.2 g, 730.4 mmol, 4.0 eq.) at 20-25° C. Then the mixture was stirred at 50° C. for 2 hours The mixture was acidified with saturated aqueous citric acid to pH=4-5 and then extracted with EtOAc. The combined organic phase was washed with brine, dried, and concentrated to the title compound as a yellow oil.

Step 9: Synthesis of tert-butyl (3-(1-(difluoro(4-methyl-4H-1,2,4-triazol-3-yl)methyl)cyclopropyl)phenyl)carbamate. To a solution of tert-butyl (3-(1-(difluoro(5-mercapto-4-methyl-4H-1,2,4-triazol-3-yl)methyl)cyclopropyl)phenyl)carbamate (72.3 g, 182.5 mmol, 1.0 eq.) in methylene chloride (400 mL) was added a solution of H₂O₂ (103.4 g, 912.9 mmol, 87.70 mL, 30% purity, 5.0 eq.) in AcOH (16.4 g, 273.8 mmol, 15.6 mL, 1.5 eq.) at 20-25° C. After the addition, the mixture was stirred at 20-25° C. for 2 hours. The mixture was adjusted to pH=8-9 with saturated aqueous sodium bicarbonate and then extracted with methylene chloride. The combined organic phase was washed with saturated aqueous sodium bicarbonate, dried, concentrated. The residue was purified by silica gel chromatography eluting with petroleum ether/ethyl acetate to give the title compound (25.0 g, 63.8 mmol, 35%) as a colorless solid.

Step 10: Synthesis of 3-(1-(difluoro(4-methyl-4H-1,2,4-triazol-3-yl)methyl)cyclopropyl)aniline. A mixture of tert-butyl (3-(l-(difluoro(4-methyl-4H-1,2,4-triazol-3-yl)methyl)cyclopropyl)phenyl)carbamate (35.0 g, 96.0 mmol, 1.0 eq.) in HCl/dioxane (4 M, 150 mL, 6.3 eq.) was stirred at 20-25° C. for 3 hours. The mixture was filtered and the filtered cake was dried under vacuum. The filtered cake was collected and dried under vacuum and then lyophilized to the title compound (27.4 g, 85.6 mmol, 89%, HCl salt):

Step 11: Synthesis of 2-cyclopropyl-N-(3-{l-[difluoro(4-methyl-4H-1,2,4-triazol-3-yl)methyl]cyclopropyl}phenyl)-6-methylpyrimidine-4-carboxamide. The amide bond formation reaction was carried out in a similar fashion as for 74 using 3-{1-[difluoro(4-methyl-4H-1,2,4-triazol-3-yl)methyl]cyclopropyl}aniline (23 mg, 0.09 mmol, 1.0 eq.) and 2-cyclopropyl-6-methylpyrimidine-4-carboxylic acid (16 mg, 0.09 mmol, 1.0 eq.) as reactants afford the title compound (11 mg, 30%) as an off-white solid: ¹H NMR (500 MHz, DMSO-d6) δ 10.38 (s, 1H), 8.44 (s, 1H), 7.81 (t, J=2.0 Hz, 1H), 7.74 (ddd, J=8.1, 2.2, 1.0 Hz, 1H), 7.63 (s, 1H), 7.22 (t, J=7.9 Hz, 1H), 7.00 (dt, J=7.7, 1.3 Hz, 1H), 3.22 (s, 3H), 2.45 (s, 3H), 2.32-2.22 (m, 1H), 1.37-1.31 (m, 2H), 1.14-0.98 (m, 6H); LCMS: C₂₂H₂₂F₂N₆O requires: 424, found: m/z=425 [M+H]⁺.

### Example 347: 2,6-dicyclopropyl-N-(3-(3-((4-methyl-4H-1,2,4-triazol-3-yl)methyl)oxetan-3-yl)phenyl)pyrimidine-4-carboxamide (347)

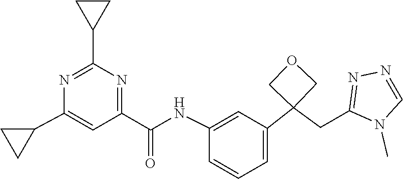 [SMILES: Cn1cnnc1CC1(c2cccc(NC(=O)c3cc(C4CC4)nc(C4CC4)n3)c2)COC1]

3-(3-((4-methyl-4H-1,2,4-triazol-3-yl)methyl)oxetan-3-yl)aniline (50 mg, 0.2 mmol, 1 eq) and 2,6-dicyclopropylpyrimidine-4-carboxylic acid (46 mg, 0.23 mmol, 1.1 eq) were coupled in a similar fashion as for 74 to afford 66 mg of the title compound as an off-white solid: ¹H NMR (500 Mz, DMSO-d₆) δ 10.40 (s, 1H), 8.64 (s, 1H), 7.79 (dd, J=8.1, 2.0 Hz, 1H), 7.73 (s, 1H), 7.49 (t, J=1.9 Hz, 1H), 7.31 (t, J=7.9 Hz, 1H), 6.76 (d, J=7.6 Hz, 1H), 4.92 (d, J=6.0 Hz, 2H), 4.87 (d, J=6.1 Hz, 2H), 3.60 (s, 2H), 3.04 (s, 3H), 2.31-2.15 (m, 2H), 1.18-0.98 (m, 8H); LCMS: C₂₄H₂₆N₆O₂ requires: 430, found: m/z=431 [M+H]⁺.

### Example 348: 2-Cyclopropyl-N-(3-(3-((4-methyl-4H-1,2,4-triazol-3-yl)methyl)oxetan-3-yl)phenyl)-6-(4-methylpiperazin-1-yl)pyrimidine-4-carboxamide (348)

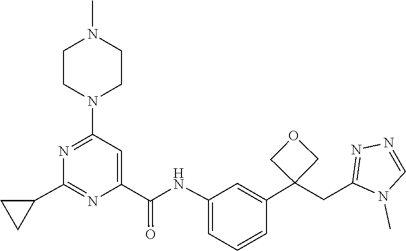 [SMILES: CN1CCN(c2cc(C(=O)Nc3cccc(C4(Cc5nncn5C)COC4)c3)nc(C3CC3)n2)CC1]

Step 1: Synthesis of 2-cyclopropyl-6-(4-methylpiperazin-1-yl)pyrimidine-4-carboxylic acid. A solution of methyl 2-cyclopropyl-6-(4-methylpiperazin-1-yl)pyrimidine-4-carboxylate (45 mg, 0.16 mmol, 1 eq.) in THF (0.58 mL) was treated with an aqueous solution of lithium hydroxide (1M, 0.20 mL, 0.20 mmol, 1.25 eq.) at rt. The mixture was stirred for 2 h at rt and quenched with hydrochloric acid (2M, 0.10 mL, 0.20 mmol, 1.25 eq.). THF was removed in vacuo and the mixture was dried on a lyophilizer to yield the title compound.

Step 2: Synthesis of 2-cyclopropyl-N-(3-(3-((4-methyl-4H-1,2,4-triazol-3-yl)methyl)oxetan-3-yl)phenyl)-6-(4-methylpiperazin-1-yl)pyrimidine-4-carboxamide. The amide bond formation reaction was carried out in a similar fashion as for 184 using the crude 2-cyclopropyl-6-(4-methylpiperazin-1-yl)pyrimidine-4-carboxylic acid, (0.16 mmol, 1 eq.) and 3-(3-((4-methyl-4H-1,2,4-triazol-3-yl)methyl)oxetan-3-yl)aniline (50 mg, 0.16 mmol, 1 eq.) as reactants afforded the title compound (12.5 mg, 16%) as an off-white solid: ¹H NMR (500 MHz, DMSO-d₆) δ 10.17 (s, 1H), 8.13 (s, 1H), 7.72 (dd, J=7.7, 1.9 Hz, 1H), 7.38 (t, J=2.0 Hz, 1H), 7.20 (t, J=7.9 Hz, 1H), 7.09 (s, 1H), 6.60 (dt, J=7.7, 1.3 Hz, 1H), 4.92-4.74 (m, 4H), 3.71-3.49 (m, 4H), 3.41 (s, 3H), 2.85 (s, 2H), 2.56-2.22 (m, 4H), 2.15 (s, 3H), 2.05 (td, J=8.2, 4.2 Hz, 1H), 1.00 (p, J=3.7, 3.3 Hz, 2H), 0.91 (dt, J=8.3, 3.2 Hz, 2H); LCMS: C₂₆H₃₂N₈O₂ requires: 488, found: m/z=489 [M+H]⁺.

### General Procedure 8:

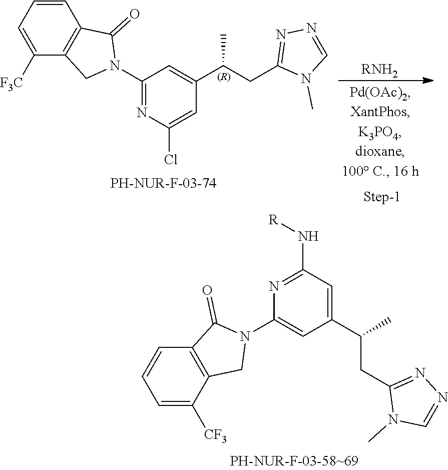

To a degassed solution of (R)-2-(6-chloro-4-(1-(4-methyl-4H-1,2,4-triazol-3-yl)propan-2-yl)pyridin-2-yl)-4-(trifluoromethyl)isoindolin-1-one (150 mg, 0.34 mmol) in dioxane (2 mL) were added RNH₂ (1.02 mmol), XantPhos (20 mg, 0.03 mmol), K₃PO₄ (146 mg, 0.69 mmol) and Pd(OAc)₂ (8 mg, 0.03 mmol). The mixture was stirred at 100° C. for 12 h under N₂ atmosphere. The solvent was removed under vacuum to afford residue, which was purified by Prep-HPLC to give the desired products.

### Example 349: 2-{4-[(2R)-1-(4-methyl-4H-1,2,4-triazol-3-yl)propan-2-yl]-6-[(3-methyloxetan-3-yl)amino]pyridin-2-yl}-4-(trifluoromethyl)-2,3-dihydro-1H-isoindol-1-one (349)

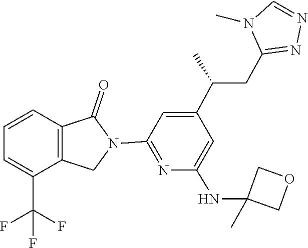 [SMILES: C[C@H](Cc1nncn1C)c1cc(NC2(C)COC2)nc(N2Cc3c(cccc3C(F)(F)F)C2=O)c1]

The coupling reaction was carried out according to General Procedure 8 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (100 mg, 0.23 mmol, 1.0 eq.) and 3-methyloxetan-3-amine (30 mg, 0.34 mmol, 1.5 eq.) as reactants to afford the title compound (7 mg, 6%) as an off-white solid: ¹H NMR (500 MHz, DMSO-d6) δ 8.36 (d, J=1.4 Hz, 1H), 8.16 (t, J=7.1 Hz, 2H), 7.87 (t, J=7.7 Hz, 1H), 6.90 (s, 1H), 6.65 (s, 1H), 5.36-5.22 (m, 2H), 5.14 (d, J=29.0 Hz, 1H), 4.39-4.28 (m, 2H), 4.08 (d, J=12.2 Hz, 1H), 3.86-3.72 (m, 1H), 3.59 (s, 3H), 3.39-3.36 (m, 1H), 3.02 (qd, J=15.5, 7.2 Hz, 2H), 1.27 (t, J=3.5 Hz, 3H), 1.04 (d, J=6.1 Hz, 3H); LCMS: C₂₄H₂₅F₃N₆O₂ requires: 486, found: m/z=487 [M+H]⁺.

### Example 350: 2-{3-[(2R)-1-(4-methyl-4H-1,2,4-triazol-3-yl)propan-2-yl]-5-[(oxetan-3-yl)amino]phenyl}-4-(trifluoromethyl)-2,3-dihydro-1H-isoindol-1-one (350)

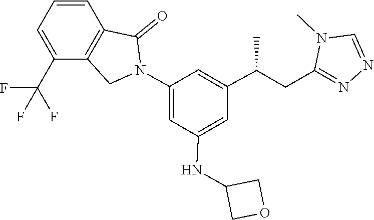 [SMILES: C[C@H](Cc1nncn1C)c1cc(NC2COC2)cc(N2Cc3c(cccc3C(F)(F)F)C2=O)c1]

Step 1: The enantiomers of Step 7 of Example 476 (0.35 g) were separated using chiral chromatography on a CHIRALPAK AD-H column with CO₂ and methanol as mobile phase to afford (R)-2-(3-bromo-5-(1-(4-methyl-4H-1,2,4-triazol-3-yl)propan-2-yl)phenyl)-4-(trifluoromethyl)isoindolin-1-one (0.15 g) and (S)-2-(3-bromo-5-(1-(4-methyl-4H-1,2,4-triazol-3-yl)propan-2-yl)phenyl)-4-(trifluoromethyl)isoindolin-1-one (0.14 g) as off-white solids.

Step 2: Synthesis of 2-{3-[(2R)-1-(4-methyl-1,2,4-triazol-3-yl)propan-2-yl]-5-(oxetan-3-ylamino)phenyl}-4-(trifluoromethyl)-3H-isoindol-1-one. To a stirring solution of 2-{3-bromo-5-[(2R)-1-(4-methyl-4H-1,2,4-triazol-3-yl)propan-2-yl]phenyl}-4-(trifluoromethyl)-2,3-dihydro-1H-isoindol-1-one (100 mg, 0.21 mmol) and oxetan-3-amine (31 mg, 0.42 mmol) in dioxane (1 mL) was added sodium 2-methylpropan-2-olate (60 mg, 0.63 mmol) and [2-(2-aminoethyl)phenyl](chloro)palladium; di-tert-butyl[2′,4′,6′-tris(propan-2-yl)-[1,1′-biphenyl]-2-yl]phosphane (29 mg, 0.04 mmol). The mixture was stirred at 120° C. for about 1 h in a sealed vial and then cooled to rt. The residue was purified by HPLC (15-98% acetonitrile in water with 0.1% trifluoroacetic acid). The solution was partitioned between 15% IPA/chloroform and saturated sodium bicarbonate. The organic layer was dried, filtered, and concentrated to give the title compound (20 mg, 20%): ¹H NMR (500 MHz, DMSO-d6) δ 8.29 (s, 1H), 8.05 (dd, J=19.8, 7.7 Hz, 2H), 7.79 (t, J=7.7 Hz, 1H), 7.02 (d, J=1.7 Hz, 2H), 6.49 (d, J=6.5 Hz, 1H), 6.18 (t, J=1.8 Hz, 1H), 5.13 (s, 2H), 4.88-4.77 (m, 2H), 4.55 (h, J=6.5 Hz, 1H), 4.41 (dt, J=8.4, 6.1 Hz, 2H), 3.44 (s, 2H), 3.15 (h, J=7.0 Hz, 1H), 3.01-2.86 (m, 2H), 1.27 (d, J=6.9 Hz, 3H); LCMS: C₂₄H₂₄F₃N₅O₂ requires: 471, found: m/z=472 [M+H]⁺.

### Example 351: 2-[6-(cyclobutylamino)-4-[(2R)-1-(4-methyl-4H-1,2,4-triazol-3-yl)propan-2-yl]pyridin-2-yl]-4-(trifluoromethyl)-2,3-dihydro-1H-isoindol-1-one (351)

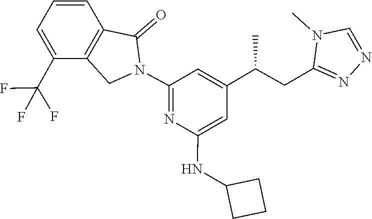 [SMILES: C[C@H](Cc1nncn1C)c1cc(NC2CCC2)nc(N2Cc3c(cccc3C(F)(F)F)C2=O)c1]

The coupling reaction was carried out according to General Procedure 8 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (100 mg, 0.23 mmol, 1.0 eq.) and cyclobutylamine (26 mg, 0.46 mmol, 2.0 eq.) as reactants to afford the title compound (8.0 mg, 7%) as an off-white solid: ¹H NMR (500 MHz, Chloroform-d) δ 8.01 (d, J=7.6 Hz, 1H), 7.88 (s, 1H), 7.82-7.73 (m, 2H), 7.62-7.49 (m, 1H), 5.82 (d, J=1.1 Hz, 1H), 5.11 (q, J=19.1 Hz, 2H), 4.61 (d, J=6.8 Hz, 1H), 4.01 (h, J=7.3 Hz, 1H), 3.38 (s, 3H), 3.28 (h, J=7.1 Hz, 1H), 2.97 (qd, J=14.9, 7.4 Hz, 2H), 2.43-2.27 (m, 2H), 1.77 (ddddd, J=22.0, 18.5, 13.1, 9.5, 7.5 Hz, 4H), 1.34 (d, J=6.9 Hz, 3H); LCMS: C₂₄H₂₅F₃N₆O requires: 470, found: m/z=471 [M+H]⁺.

### Example 352: 2-{4-[(2R)-1-(4-methyl-4H-1,2,4-triazol-3-yl)propan-2-yl]-6-[(oxetan-3-yl)amino]pyridin-2-yl}-4-(trifluoromethyl)-2,3-dihydro-1H-isoindol-1-one (352)

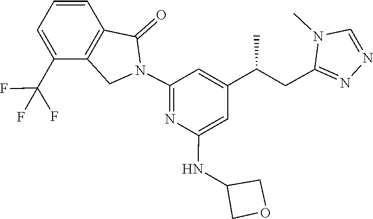 [SMILES: C[C@H](Cc1nncn1C)c1cc(NC2COC2)nc(N2Cc3c(cccc3C(F)(F)F)C2=O)c1]

The coupling reaction was carried out according to General Procedure 8 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (100 mg, 0.23 mmol, 1.0 eq.) and oxetan-3-amine (17 mg, 0.23 mmol, 1.0 eq.) as reactants to afford the title compound (12.0 mg, 11%) as an off-white solid: 1H NMR (500 MHz, Chloroform-d) δ 8.01 (d, J=7.6 Hz, 1H), 7.90 (s, 1H), 7.82 (d, J=1.1 Hz, 1H), 7.79 (dt, J=7.7, 0.9 Hz, 1H), 7.62-7.55 (m, 1H), 5.98 (d, J=1.1 Hz, 1H), 5.10 (d, J=5.9 Hz, 2H), 4.92 (td, J=6.7, 1.4 Hz, 2H), 4.86 (ddt, J=13.1, 7.0, 5.7 Hz, 1H), 4.52 (td, J=6.0, 2.1 Hz, 2H), 3.43 (s, 3H), 3.29 (p, J=7.1 Hz, 1H), 2.97 (dd, J=7.4, 1.4 Hz, 2H), 1.33 (d, J=6.9 Hz, 3H); LCMS: C₂₃H₂₃F₃N₆O₂ requires: 472, found: m/z=473 [M+H]⁺.

### Example 353: 2-{4-[(2R)-1-(4-methyl-4H-1,2,4-triazol-3-yl)propan-2-yl]-6-[(propan-2-yl)amino]pyridin-2-yl}-4-(trifluoromethyl)-2,3-dihydro-1H-isoindol-1-one (353)

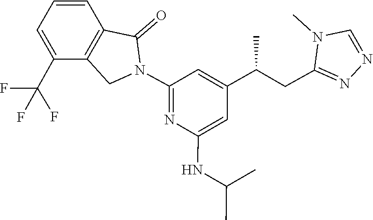 [SMILES: CC(C)Nc1cc([C@H](C)Cc2nncn2C)cc(N2Cc3c(cccc3C(F)(F)F)C2=O)n1]

The coupling reaction was carried out according to General Procedure 8 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (200 mg, 0.46 mmol, 1.0 eq.) and propan-2-amine (41 mg, 0.69 mmol, 1.5 eq.) as reactants to afford the title compound (30.0 mg, 14%) as an off-white solid: ¹H NMR (500 MHz, DMSO-d6) δ 8.31 (s, 1H), 8.06 (dd, J=22.3, 7.7 Hz, 2H), 7.79 (t, J=7.6 Hz, 1H), 7.61 (d, J=1.1 Hz, 1H), 6.41 (d, J=7.2 Hz, 1H), 6.16 (d, J=1.2 Hz, 1H), 5.19 (s, 2H), 3.94 (h, J=6.6 Hz, 1H), 3.32 (s, 3H), 3.16 (h, J=6.9 Hz, 1H), 2.95 (qd, J=15.2, 7.4 Hz, 2H), 1.26 (d, J=6.9 Hz, 3H), 1.19 (dd, J=6.4, 1.5 Hz, 6H); LCMS: C₂₃H₂₅F₃N₆O requires: 458, found: m/z=459 [M+H]⁺.

### Example 354: 2-{4-[(2R)-1-(4-methyl-4H-1,2,4-triazol-3-yl)propan-2-yl]-6-(oxetan-3-yloxy)pyridin-2-yl}-4-(trifluoromethyl)-2,3-dihydro-1H-isoindol-1-one (354)

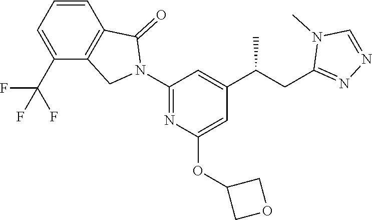 [SMILES: C[C@H](Cc1nncn1C)c1cc(OC2COC2)nc(N2Cc3c(cccc3C(F)(F)F)C2=O)c1]

The coupling reaction was carried according to General Procedure 8 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (150 mg, 0.34 mmol, 1.0 eq.) and oxetan-3-ol (76 mg, 1.03 mmol, 3.0 eq.) as reactants afford the title compound (52 mg, 32%) as an off-white solid: 1H NMR (500 MHz, DMSO-d6) δ 8.32 (s, 1H), 8.15-8.06 (m, 3H), 7.82 (t, J=7.7 Hz, 1H), 6.70 (s, 1H), 5.59 (p, J=5.8 Hz, 1H), 5.19 (s, 2H), 4.92 (t, J=6.9 Hz, 2H), 4.66 (ddd, J=7.4, 5.5, 1.5 Hz, 2H), 4.35 (d, J=4.2 Hz, 1H), 3.78 (pd, J=6.1, 4.2 Hz, 1H), 3.58 (s, 2H), 3.11-2.96 (m, 2H), 1.05 (d, J=6.1 Hz, 3H); LCMS: C₂₃H₂₂F₃N₅O₃ requires: 473, found: m/z=474 [M+H]⁺.

### Example 355: 2-(4-((R)-1-(4-methyl-4H-1,2,4-triazol-3-yl)propan-2-yl)-6-((2-methyloxetan-3-yl)oxy)pyridin-2-yl)-4-(trifluoromethyl)isoindolin-1-one (355)

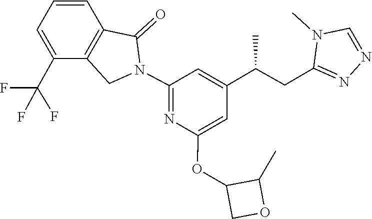 [SMILES: CC1OCC1Oc1cc([C@H](C)Cc2nncn2C)cc(N2Cc3c(cccc3C(F)(F)F)C2=O)n1]

The coupling reaction was carried out according to General Procedure 8 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (250 mg, 0.57 mmol, 1.0 eq.) and 2-methyloxetan-3-ol (51 mg, 0.57 mmol, 1.0 eq.) as reactants afford the title compound (110 mg, 39%) as an off-white solid: 1H NMR (500 MHz, Chloroform-d) δ 8.18 (dd, J=4.5, 1.1 Hz, 1H), 8.12 (d, J=7.6 Hz, 1H), 8.02 (d, J=1.9 Hz, 1H), 7.90 (d, J=7.7 Hz, 1H), 7.70 (t, J=7.7 Hz, 1H), 6.56-6.47 (m, 1H), 5.70-5.58 (m, 1H), 5.28-5.07 (m, 3H), 5.05-4.94 (m, 1H), 4.92-4.84 (m, 1H), 4.64 (dd, J=7.2, 5.9 Hz, 1H), 3.58 (dd, J=6.5, 1.2 Hz, 2H), 3.50 (p, J=7.1 Hz, 1H), 3.14-3.02 (m, 2H), 1.62-1.60 (m, 3H), 1.49-1.40 (m, 3H); LCMS: C₂₄H₂₄F₃N₅O₃ requires: 487, found: m/z=488 [M+H]⁺.

### Example 356: (R)-2-(6-(2,2-difluoroethoxy)-4-(1-(4-methyl-4H-1,2,4-triazol-3-yl)propan-2-yl)pyridin-2-yl)-4-(trifluoromethyl)isoindolin-1-one (356)

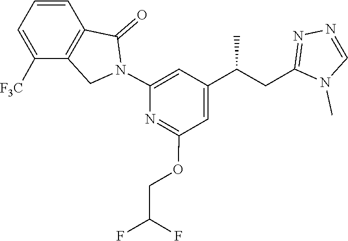 [SMILES: C[C@H](Cc1nncn1C)c1cc(OCC(F)F)nc(N2Cc3c(cccc3C(F)(F)F)C2=O)c1]

(R)-2-(6-chloro-4-(1-(4-methyl-4H-1,2,4-triazol-3-yl)propan-2-yl)pyridin-2-yl)-4-(trifluoromethyl)isoindolin-1-one (50 mg, 0.11 mmol, 1 eq) and 2,2-difluoroethan-1-ol (50 mg, 0.57 mmol, 5 eq) were coupled according to General Procedure 8 to afford 12 mg of the title compound as an off-white solid: ¹H NMR (500 MHz, DMSO-d₆) δ 8.88 (s, 1H), 8.36-7.92 (m, 3H), 7.82 (t, J=7.7 Hz, 1H), 6.77 (d, J=1.2 Hz, 1H), 6.49 (tt, J=54.7, 3.5 Hz, 1H), 5.34-5.20 (m, 2H), 4.62 (td, J=15.0, 3.5 Hz, 2H), 3.39 (h, J=7.0 Hz, 1H), 3.29-3.09 (m, 2H), 1.33 (d, J=6.9 Hz, 3H); LCMS: C₂₂H₂₀F₅N₅O₂ requires: 481, found: m/z=482 [M+H]⁺.

### Example 357: (R)-2-(6-(2-hydroxyethylamino)-4-(1-(4-methyl-4H-1,2,4-triazol-3-yl)propan-2-yl)pyridin-2-yl)-4-(trifluoromethyl)isoindolin-1-one (357)

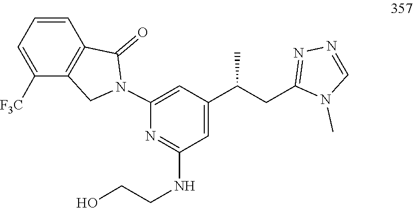 [SMILES: C[C@H](Cc1nncn1C)c1cc(NCCO)nc(N2Cc3c(cccc3C(F)(F)F)C2=O)c1]

Step 1: Synthesis of (R)-2-(6-(2-(benzyloxy)ethylamino)-4-(1-(4-methyl-4H-1,2,4-triazol-3-yl)propan-2-yl)pyridin-2-yl)-4-(trifluoromethyl)isoindolin-1-one. Followed General procedure 8 with 2-(benzyloxy)ethan-1-amine as a substrate to afford crude product, which was purified by flash column chromatography with 0˜5% methanol in dichloromethane to afford the title compound (233 mg, 61%) as a yellow foam. MS (ESI) calculated for (C₂₉H₂₉F₃N₆O₂) [M+H]⁺, 551.2; found, 551.1.

Step 2: Synthesis of (R)-2-(6-(2-hydroxyethylamino)-4-(1-(4-methyl-4H-1,2,4-triazol-3-yl)propan-2-yl)pyridin-2-yl)-4-(trifluoromethyl)isoindolin-1-one (357). To a mixture of (R)-2-(6-(2-(benzyloxy)ethylamino)-4-(1-(4-methyl-4H-1,2,4-triazol-3-yl)propan-2-yl)pyridin-2-yl)-4-(trifluoromethyl)isoindolin-1-one (233 mg, 0.42 mmol) in methanol (3 mL) was added Pd(OH)₂/C (80 mg). The mixture was stirred at 50° C. for 16 h under H₂ atmosphere. The solids were filtered out. The filtrate was concentrated. The residue was purified by Prep-HPLC to the title compound (45 mg, 23%) as an off-white solid. MS (ESI) calculated for (C₂₂H₂₃F₃N₅O₂) [M+H]⁺, 461.2; found, 461.1. ¹H NMR (300 MHz, DMSO-d₆) δ 8.31 (s, 1H), 8.13-8.00 (m, 2H), 7.79 (t, J=7.8 Hz, 1H), 7.64 (s, 1H), 6.52 (t, J=5.7 Hz, 1H), 6.22 (s, 1H), 5.18 (s, 2H), 4.67 (t, J=5.4 Hz, 1H), 3.58 (s, 3H), 3.64-3.51 (m, 2H), 3.35 (d, J=5.7 Hz, 1H), 3.31 (d, J=6.0 Hz, 1H), 3.22-3.10 (m, 1H), 3.06-2.86 (m, 2H), 1.26 (d, J=6.9 Hz, 3H).

### Example 358: (R)-2-(6-ethoxy-4-(1-(4-methyl-4H-1,2,4-triazol-3-yl)propan-2-yl)pyridin-2-yl)-4-(trifluoromethyl)isoindolin-1-one (358)

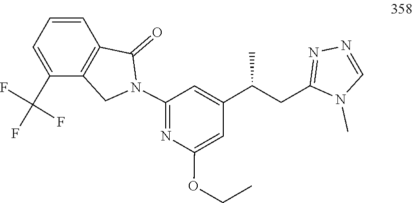 [SMILES: CCOc1cc([C@H](C)Cc2nncn2C)cc(N2Cc3c(cccc3C(F)(F)F)C2=O)n1]

Followed General procedure 8 with ethanol as a substrate to afford crude product, which was purified by Prep-HPLC to afford the title compound (15 mg, 15%) as an off-white solid. MS (ESI) calculated for (C₂₂H₂₂F₃N₅O₂) [M+H]⁺, 446.2, found, 446.2. ¹H NMR (300 MHz, CDCl₃) δ 8.31 (s, 1H), 8.16-7.94 (m, 3H), 7.80 (t, J=7.8 Hz, 1H), 6.58 (s, 1H), 5.23 (s, 2H), 4.34 (q, J=6.9 Hz, 2H), 3.57 (s, 3H), 3.36-3.29 (m, 1H), 3.05-3.01 (m, 2H), 1.64-1.12 (m, 6H).

### Example 359: (R)-2-(6-(2-hydroxyethoxy)-4-(1-(4-methyl-4H-1,2,4-triazol-3-yl)propan-2-yl)pyridin-2-yl)-4-(trifluoromethyl)isoindolin-1-one (359)

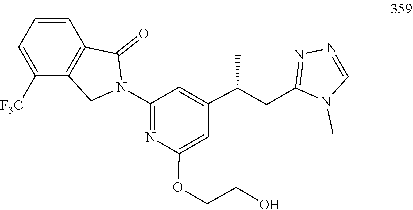 [SMILES: C[C@H](Cc1nncn1C)c1cc(OCCO)nc(N2Cc3c(cccc3C(F)(F)F)C2=O)c1]

Followed General procedure 8 with ethane-1,2-diol as a substrate to afford crude product, which was purified by Prep-HPLC to afford the title compound (24 mg, 23%) as a colorless solid. MS (ESI) calculated for (C₂₂H₂₂F₃N₅O₃) [M+H]⁺, 462.2; found, 462.1. ¹H NMR (300 MHz, DMSO-d₆) δ 8.31 (s, 1H), 8.14-8.01 (m, 3H), 7.81-7.78 (m, 1H), 6.60 (s, 1H), 5.22 (s, 2H), 4.87 (br, 1H), 4.30 (t, J=5.1 Hz, 2H), 3.76 (t, J=5.1 Hz, 2H), 3.58 (s, 3H), 3.31 (d, J=12 Hz, 1H), 3.13-2.93 (m, 2H), 1.30 (d, J=6.9 Hz, 3H).

### Example 360: (R)-2-(6-(ethylamino)-4-(1-(4-methyl-4H-1,2,4-triazol-3-yl)propan-2-yl)pyridin-2-yl)-4-(trifluoromethyl)isoindolin-1-one (360)

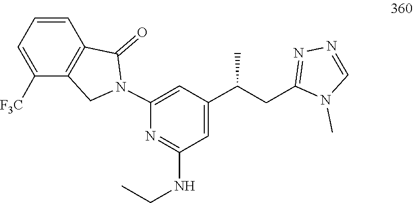 [SMILES: CCNc1cc([C@H](C)Cc2nncn2C)cc(N2Cc3c(cccc3C(F)(F)F)C2=O)n1]

Followed General procedure 8 with ethylamine as a substrate to afford crude product, which was purified by Prep-HPLC to the title compound (20 mg, 14%) as an off-white solid. MS (ESI) calculated for (C₂₂H₂₃F₃N₆O) [M+H]⁺, 445.2; found, 445.5. ¹H NMR (300 MHz, DMSO-d₆) δ 8.31 (s, 1H), 8.09-8.02 (m, 2H), 7.79 (t, J=6.0 Hz, 1H), 7.63 (s, 1H), 6.55 (t, J=3.0 Hz, 1H), 6.15 (s, 1H), 5.19 (s, 2H), 3.55 (s, 3H), 3.38-3.09 (m, 3H), 3.06-2.86 (m, 2H), 1.26 (d, J=6.9 Hz, 3H), 1.17 (t, J=6.9 Hz, 3H).

### Example 361: 2-(4-((R)-1-(4-methyl-4H-1,2,4-triazol-3-yl)propan-2-yl)-6-((R)-THF-3-ylamino)pyridin-2-yl)-4-(trifluoromethyl)isoindolin-1-one (361)

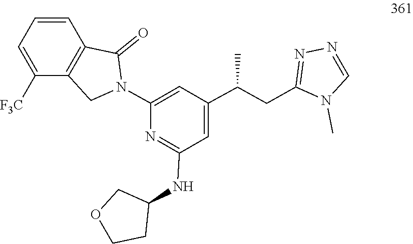 [SMILES: C[C@H](Cc1nncn1C)c1cc(N[C@H]2CCOC2)nc(N2Cc3c(cccc3C(F)(F)F)C2=O)c1]

Followed General procedure 8 with (R)-THF-3-amine as a substrate to afford crude product, which was purified by Prep-HPLC to the title compound (24 mg, 14%) as a colorless solid. MS (ESI) calculated for (C₂₄H₂₅F₃N₆O₂) [M+H]⁺, 487.2; found, 487.5. ¹H NMR (300 MHz, DMSO-d₆) δ 8.31 (s, 1H), 8.10-8.03 (m, 2H), 7.79 (t, J=9.0 Hz, 1H), 1 FI (s, 1H), 6.85 (d, J=3.0 Hz, 1H), 6.22 (s, 1H), 5.20 (s, 2H), 4.34-4.24 (m, 1H), 3.97-3.92 (m, 1H), 3.90-3.68 (m, 2H), 3.65-3.47 (m, 1H), 3.59 (s, 3H), 3.20-3.13 (m, 1H), 3.06-2.86 (m, 2H), 2.29-2.05 (m, 1H), 1.94-1.78 (m, 1H), 1.27 (d, J=6.0 Hz, 3H).

### Example 362: 2-(4-((R)-1-(4-methyl-4H-1,2,4-triazol-3-yl)propan-2-yl)-6-((S)-THF-3-ylamino)pyridin-2-yl)-4-(trifluoromethyl)isoindolin-1-one (362)

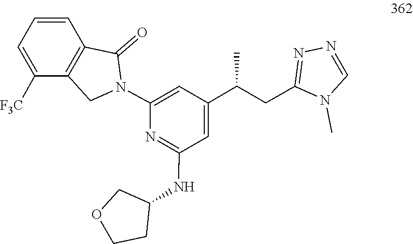 [SMILES: C[C@H](Cc1nncn1C)c1cc(N[C@@H]2CCOC2)nc(N2Cc3c(cccc3C(F)(F)F)C2=O)c1]

Followed General procedure 8 with (S)-THF-3-amine as a substrate to afford crude product, which was purified by Prep-HPLC to afford the title compound (58 mg, 35%) as a colorless solid. MS (ESI) calculated for (C₂₄H₂₅F₃N₆O₂) [M+H]⁺, 487.2; found, 487.4. ¹H NMR (300 MHz, DMSO-d₆) δ 8.31 (s, 1H), 8.10-8.02 (m, 2H), 7.85-7.74 (m, 1H), 7.67 (s, 1H), 6.85 (d, J=6.0 Hz, 1H), 6.21 (d, J=3.0 Hz, 1H), 5.27-5.13 (m, 2H), 4.33-4.24 (m, 1H), 3.97-3.92 (m, 1H), 3.90-3.68 (m, 2H), 3.62-3.51 (m, 1H), 3.59 (s, 3H), 3.24-3.11 (m, 1H), 3.06-2.83 (m, 2H), 2.29-2.05 (m, 1H), 1.94-1.78 (m, 1H), 1.27 (d, J=6.6 Hz, 3H).

### Example 363: (R)-2-(4-(1-(4-methyl-4H-1,2,4-triazol-3-yl)propan-2-yl)-6-(methylamino)pyridin-2-yl)-4-(trifluoromethyl)isoindolin-1-one (363)

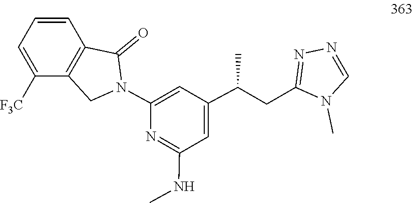 [SMILES: CNc1cc([C@H](C)Cc2nncn2C)cc(N2Cc3c(cccc3C(F)(F)F)C2=O)n1]

Followed General procedure 8 with methylamine as a substrate to afford crude product, which was purified by Prep-HPLC to afford the title compound (33 mg, 22%) as an off-white solid. MS (ESI) calculated for (C₂₁H₂₁F₃N₆O) [M+H]⁺, 431.2; found, 431.1. ¹H NMR (300 MHz, DMSO-d₆) δ 8.31 (s, 1H), 8.10-8.02 (m, 2H), 7.82-7.76 (m, 1H), 7.65 (d, J=1.2 Hz, 1H), 6.57-6.61 (m, 1H), 6.14 (d, J=1.5 Hz, 1H), 5.20 (s, 2H), 3.54 (s, 3H), 3.23-3.12 (m, 1H), 3.06-2.88 (m, 2H), 2.79 (d, J=4.8 Hz, 3H), 1.27 (d, J=9.0 Hz, 3H).

### Example 364: 2-(6-((S)-2-hydroxypropylamino)-4-((R)-1-(4-methyl-4H-1,2,4-triazol-3-yl)propan-2-yl)pyridin-2-yl)-4-(trifluoromethyl)isoindolin-1-one (364)

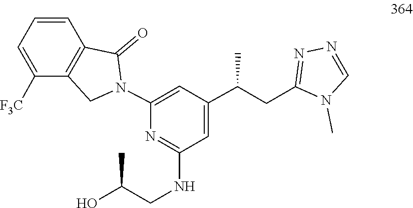 [SMILES: C[C@H](O)CNc1cc([C@H](C)Cc2nncn2C)cc(N2Cc3c(cccc3C(F)(F)F)C2=O)n1]

Followed General procedure 8 with (S)-1-aminopropan-2-ol as a substrate to afford crude product, which was purified by Prep-HPLC to afford the title compound (28 mg, 13%) as a colorless solid. MS (ESI) calculated for (C₂₃H₂₅F₃N₆O₂) [M+H]⁺, 475.2; found, 475.1. ¹H NMR (300 MHz, DMSO-d₆) δ 8.31 (s, 1H), 8.10-7.99 (m, 2H), 7.81-7.78 (m, 1H), 7.62 (s, 1H), 6.56 (t, J=6.0 Hz, 1H), 6.24 (s, 1H), 5.18 (s, 2H), 4.67 (d, J=4.5 Hz, 1H), 3.85 (s, 1H), 3.55 (s, 3H), 3.33 (s, 1H), 3.28-3.04 (m, 2H), 3.03-2.85 (m, 2H), 1.26 (d, J=6.6 Hz, 3H), 1.11 (d, J=6.9 Hz, 3H).

### Example 365: 2-(6-((S)-1-hydroxypropan-2-ylamino)-4-((R)-1-(4-methyl-4H-1,2,4-triazol-3-yl)propan-2-yl)pyridin-2-yl)-4-(trifluoromethyl)isoindolin-1-one (365)

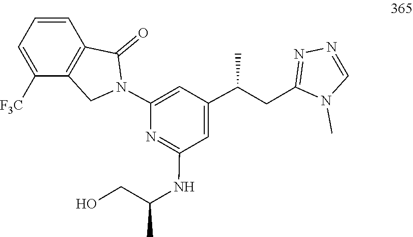 [SMILES: C[C@H](Cc1nncn1C)c1cc(N[C@@H](C)CO)nc(N2Cc3c(cccc3C(F)(F)F)C2=O)c1]

Followed General procedure 8 with (S)-2-aminopropan-1-ol as a substrate to afford crude product, which was purified by Prep-HPLC to afford the title compound (21 mg, 9%) as an off-white solid. MS (ESI) calculated for (C₂₃H₂₅F₃N₆O₂) [M+H]⁺, 475.2; found, 475.1. ¹H NMR (300 MHz, DMSO-d₆) δ 8.31 (s, 1H), 8.11-8.02 (m, 2H), 7.79 (t, J=8.1 Hz, 1H), 7.62 (s, 1H), 6.30 (d, J=7.8 Hz, 1H), 6.22 (s, 1H), 5.18 (s, 2H), 4.67 (t, J=5.4 Hz, 1H), 3.93-3.82 (m, 1H), 3.56 (s, 3H), 3.53-3.45 (m, 1H), 3.42-3.34 (m, 1H), 3.22-3.11 (m, 1H), 3.06-2.85 (m, 2H), 1.26 (d, J=7.2 Hz, 3H), 1.16 (d, J=6.9 Hz, 3H).

### Example 366: 2-(6-((R)-1-hydroxypropan-2-ylamino)-4-((R)-1-(4-methyl-4H-1,2,4-triazol-3-yl)propan-2-yl)pyridin-2-yl)-4-(trifluoromethyl)isoindolin-1-one (366)

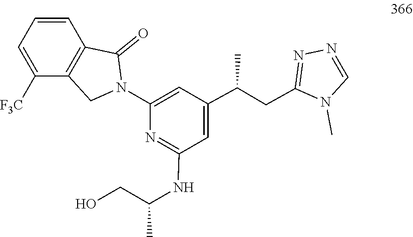 [SMILES: C[C@H](CO)Nc1cc([C@H](C)Cc2nncn2C)cc(N2Cc3c(cccc3C(F)(F)F)C2=O)n1]

Followed General procedure 8 with (R)-2-aminopropan-1-ol as a substrate to afford crude product, which was purified by Prep-HPLC to afford the title compound (6.6 mg, 3%) as an off-white solid. MS (ESI) calculated for (C₂₃H₂₅F₃N₆O₂) [M+H]⁺, 475.2; found, 475.1. ¹H NMR (300 MHz, DMSO-d₆) δ 8.31 (s, 1H), 8.13-7.99 (m, 2H), 7.81-7.77 (m, 1H), 7.65-7.60 (m, 1H), 6.30 (d, J=7.5 Hz, 1H), 6.21 (d, J=1.2 Hz, 1H), 5.18 (s, 2H), 4.68 (t, J=5.7 Hz, 1H), 3.93-3.83 (m, 1H), 3.56 (s, 3H), 3.54-3.45 (m, 1H), 3.43-3.32 (m, 1H), 3.21-3.10 (m, 1H), 3.06-2.87 (m, 2H), 1.26 (d, J=6.9 Hz, 3H), 1.15 (d, J=6.9 Hz, 3H).

### Example 367: (R)-2-(3-(1-(4-Methylisoxazol-3-yl)propan-2-yl)phenyl)-4-(trifluoromethyl)isoindolin-1-one (367a) and (S)-2-(3-(1-(4-methylisoxazol-3-yl)propan-2-yl)phenyl)-4-(trifluoromethyl)isoindolin-1-one (367b)

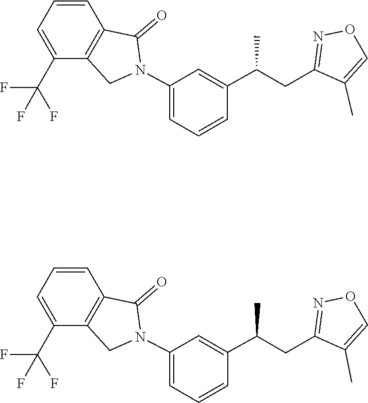 [SMILES: Cc1conc1C[C@@H](C)c1cccc(N2Cc3c(cccc3C(F)(F)F)C2=O)c1 ; Cc1conc1C[C@H](C)c1cccc(N2Cc3c(cccc3C(F)(F)F)C2=O)c1]

The enantiomers of racemate 268 (0.35 g) were separated using chiral chromatography on a CHIRALPAK AD-H column with CO₂ and methanol as mobile phase to afford.

(R)-2-(3-(1-(4-Methylisoxazol-3-yl)propan-2-yl)phenyl)-4-(trifluoromethyl)isoindolin-1-one (150 mg, colorless solid): ¹H NMR (500 MHz, DMSO-d₆) δ 8.50 (d, J=1.2 Hz, 1H), 8.12-8.01 (m, 2H), 7.89-7.74 (m, 3H), 7.37 (t, J=7.9 Hz, 1H), 7.13 (dt, J=7.7, 1.3 Hz, 1H), 5.22 (d, J=1.7 Hz, 2H), 3.20 (p, J=7.1 Hz, 1H), 2.92 (d, J=7.5 Hz, 2H), 1.89 (d, J=1.2 Hz, 3H), 1.29 (d, J=7.0 Hz, 3H); LCMS: C₂₂H₁₉F₃N₂O₂ requires: 400, found: m/z=401 [M+H]⁺.

(S)-2-(3-(1-(4-methylisoxazol-3-yl)propan-2-yl)phenyl)-4-(trifluoromethyl)isoindolin-1-one (140 mg, colorless solid): ¹H NMR (500 MHz, DMSO-d₆) δ 8.50 (d, J=1.2 Hz, 1H), 8.12-8.02 (m, 2H), 7.91-7.74 (m, 3H), 7.37 (t, J=7.9 Hz, 1H), 7.13 (dt, J=7.7, 1.2 Hz, 1H), 5.22 (d, J=1.6 Hz, 2H), 3.21 (h, J=7.1 Hz, 1H), 2.92 (d, J=7.5 Hz, 2H), 1.89 (d, J=1.1 Hz, 3H), 1.29 (d, J=6.9 Hz, 3H); LCMS: C₂₂H₁₉F₃O₂ requires: 400, found: m/z=401 [M+H]⁺.

### Example 368: (R)-2-(3-(1-(1,3,4-thiadiazol-2-yl)propan-2-yl)phenyl)-4-(trifluoromethyl)isoindolin-1-one (368)

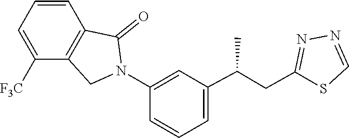 [SMILES: C[C@H](Cc1nncs1)c1cccc(N2Cc3c(cccc3C(F)(F)F)C2=O)c1]

3-[(2R)-1-(1,3,4-thiadiazol-2-yl)propan-2-yl]aniline (65 mg, 0.30 mmol, 1 eq) and methyl 2-(bromomethyl)-3-(trifluoromethyl)benzoate (88 mg, 0.30 mmol, 1 eq) were coupled in a manner similar to 260, step 2 to afford 33 mg of the title compound as an off-white solid: ¹H NMR (500 MHz, DMSO-d₆) δ 9.41 (s, 1H), 8.08 (d, J=7.6 Hz, 1H), 8.04 (d, J=7.7 Hz, 1H), 7.88-7.76 (m, 3H), 7.37 (t, J=7.9 Hz, 1H), 7.15 (dt, J=7.7, 1.2 Hz, 1H), 5.21 (s, 2H), 3.29 (q, J=7.2 Hz, 1H), 1.32 (d, J=7.0 Hz, 3H); LCMS: C₂₀H₁₆F₃N₃O requires: 403, found: m/z=404 [M+H]⁺.

### Example 369: 2-{3-[(1S,2R)-2-(4-methyl-4H-1,2,4-triazol-3-yl)cyclopropyl]phenyl}-4-(trifluoromethyl)-1H,2H,3H-pyrrolo[3,4-c]pyridin-1-one (369)

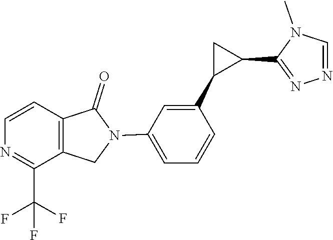 [SMILES: Cn1cnnc1[C@@H]1C[C@@H]1c1cccc(N2Cc3c(ccnc3C(F)(F)F)C2=O)c1]

The isoindolone formation reaction was carried out in a manner similar to 260, step 2 using 3-[(1S,2R)-2-(4-methyl-4H-1,2,4-triazol-3-yl)cyclopropyl]aniline (100 mg, 0.47 mmol, 1.0 eq.) and methyl 2-(bromomethyl)-3-(trifluoromethyl)benzoate (140 mg, 0.47 mmol, 1.0 eq.) as reactants afford the title compound (120 mg, 64%) as an off-white solid: ¹H NMR (500 MHz, Chloroform-d) δ 8.86 (d, J=4.8 Hz, 1H), 7.92 (d, J=4.8 Hz, 1H), 7.77 (s, 1H), 7.57-7.39 (m, 2H), 7.14 (t, J=7.9 Hz, 1H), 6.81 (dt, J=7.8, 1.2 Hz, 1H), 4.94 (d, J=1.8 Hz, 2H), 3.37 (s, 3H), 2.59 (td, J=8.6, 6.7 Hz, 1H), 2.29 (td, J=8.7, 5.9 Hz, 1H), 2.15 (q, J=6.0 Hz, 1H), 1.65 (td, J=8.7, 5.9 Hz, 1H); LCMS: C₂₀H₁₆F₃N₅O requires: 399, found: m/z=400 [M+H]⁺.

### Example 370: 4,6-dicyclopropyl-2-{3-[(2R)-1-(4-methyl-1,2,4-triazol-3-yl)propan-2-yl]phenyl}-3H-pyrrolo[3,4-c]pyridin-1-one (370)

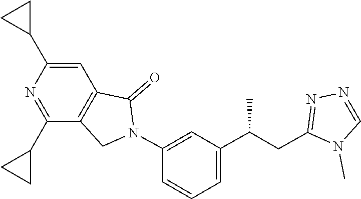 [SMILES: C[C@H](Cc1nncn1C)c1cccc(N2Cc3c(cc(C4CC4)nc3C3CC3)C2=O)c1]

Step 1. Synthesis of 4,6-dicyclopropyl-2H,3H-pyrrolo[3,4-c]pyridin-1-one. A mixture of 4,6-dichloro-1H,2H,3H-pyrrolo[3,4-c]pyridin-1-one (200 mg, 1.0 mmol, 1 eq), bromo(cyclopropyl)zinc (5.9 mL of 0.5M solution in THF, 2.7 mmol, 3 eq), palladium (II) acetate (22 mg, 0.1 mmol, 0.1 eq), 2′-(dicyclohexylphosphanyl)-N2,N2,N6,N6-tetramethyl-[1,1′-biphenyl]-2,6-diamine (86 mg, 0.2 mmol, 0.2 eq) and THF (2 mL) was heated at 65-75° C. for 18 h. After cooling to rt, the reaction was subject to aqueous work-up and chromatography on silica gel to afford 91 mg of the title compound as an off-white solid.

Step 2. Synthesis of 4,6-dicyclopropyl-2-{3-[(2R)-1-(4-methyl-1,2,4-triazol-3-yl)propan-2-yl]phenyl}-3H-pyrrolo[3,4-c]pyridin-1-one. 4,6-dicyclopropyl-1H,2H,3H-pyrrolo[3,4-c]pyridin-1-one (40 mg, 0.14 mmol, 1 eq.) and 3-[(2R)-2-(3-bromophenyl)propyl]-4-methyl-4H-1,2,4-triazole (30 mg, 0.14 mmol, 1 eq.) were coupled in a manner similar to 168, step 1 to afford the title compound (39 mg) as an off-white solid: ¹H NMR (500 MHz, DMSO-d₆)δ 9.01 (s, 1H), 7.88-7.78 (m, 2H), 7.44-7.30 (m, 2H), 7.13 (dt, J=7.8, 1.2 Hz, 1H), 5.19-5.02 (m, 3H), 3.40-3.28 (m, 1H), 3.28-3.15 (m, 2H), 2.24-2.10 (m, 2H), 1.36 (d, J=6.8 Hz, 3H), 1.10-0.99 (m, 4H), 0.99-0.83 (m, 4H); LCMS: C₂₅H₂₇N₅O requires: 413, found: m/z=414 [M+H]⁺.

### Example 371: (R)-2-(3-(1-(4-methyl-4H-1,2,4-triazol-3-yl)propan-2-yl)phenyl)-4-(trifluoromethyl)-2,3-dihydro-1H-pyrrolo[3,4-c]pyridin-1-one (371)

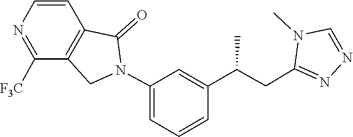 [SMILES: C[C@H](Cc1nncn1C)c1cccc(N2Cc3c(ccnc3C(F)(F)F)C2=O)c1]

The indolone formation reaction was carried out in a manner similar to 260, step 2 using (R)-3-(1-(4-methyl-4H-1,2,4-triazol-3-yl)propan-2-yl)aniline (250 mg, 1.16 mmol) and (Example X) (378 mg, 1.27 mmol) to afford the title compound (156 mg, 34%) as a light yellow solid. MS (ESI) calculated for (C₂₀H₁₈F₃N₅O) [M+H]⁺, 402.1; found, 402.1. ¹H NMR (400 MHz, DMSO-d₆) δ 8.98 (d, J=4.8 Hz, 1H), 8.28 (s, 1H), 8.11 (d, J=4.8 Hz, 1H), 7.86-7.76 (m, 2H), 7.39 (t, J=8.0 Hz, 1H), 7.17 (d, J=7.6 Hz, 1H), 5.32 (s, 2H), 3.46 (s, 3H), 3.39-3.33 (m, 1H), 3.07-2.97 (m, 2H), 1.32 (d, J=6.8 Hz, 3H).

### Example 372: 4-cyclopropyl-6-methyl-2-{3-[(2R)-1-(4-methyl-4H-1,2,4-triazol-3-yl)propan-2-yl]phenyl}-1H,2H,3H-pyrrolo[3,4-c]pyridin-1-one (372)

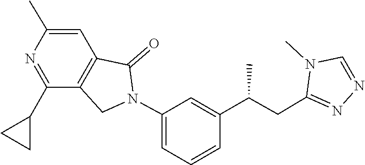 [SMILES: Cc1cc2c(c(C3CC3)n1)CN(c1cccc([C@H](C)Cc3nncn3C)c1)C2=O]

Step 1: Synthesis of ethyl 3-cyano-2-cyclopropyl-6-methylpyridine-4-carboxylate. A solution of ethyl 2-chloro-3-cyano-6-methylpyridine-4-carboxylate (5.0 g, 22.2 mmol) and tetrakis(triphenylphosphane) palladium (2.57 g, 2.23 mmol, 0.1 eq) in THF (150 mL) was added bromo(cyclopropyl)zinc (33 mL of 0.5 M solution in THF, 66.7 mmol). The solution was heated at 60-65° C. for 19 h. The solution was allowed to cool to rt, then poured into saturated aqueous ammonium chloride and extracted with EtOAc. The combined organic phases were dried, filtered and concentrated onto Celite. The crude material was purified by silica gel column chromatography using EtOAc in hexanes 0-60% to give the title compound (4.6 g, 90%).

Step 2: Synthesis of 4-cyclopropyl-6-methyl-2H,3H-pyrrolo[3,4-c]pyridin-1-one. To a solution of ethyl 3-cyano-2-cyclopropyl-6-methylpyridine-4-carboxylate (1.0 g, 4.34 mmol) in ethanol (50 mL) under nitrogen was added raney nickel (slurry, 0.2 mL). The reaction was put under a hydrogen balloon and stirred for 3 hours. The suspension is then filtered over celite and concentrated. The crude material was purified by silica gel column chromatography using methanol in dichloromethane 0-10% to give the title compound (620 mg, 76%).

Step 3: 4-cyclopropyl-6-methyl-2-{3-[(2R)-1-(4-methyl-4H-1,2,4-triazol-3-yl)propan-2-yl]phenyl}-1H,2H,3H-pyrrolo[3,4-c]pyridin-1-one. The coupling reaction was carried out in a manner similar to 168, step 1 using 3-[(2R)-2-(3-bromophenyl)propyl]-4-methyl-4H-1,2,4-triazole (240 mg, 0.86 mmol, 1.0 eq.) and 4-cyclopropyl-6-methyl-1H,2H,3H-pyrrolo[3,4-c]pyridin-1-one (241 mg, 1.28 mmol, 1.5 eq.) as reactants afford the title compound (120 mg, 36%) as an off-white solid: ¹H NMR (500 MHz, Methanol-d4) δ 8.25 (s, 1H), 7.72 (d, J=1.3 Hz, 1H), 7.37 (t, J=7.8 Hz, 1H), 7.25 (t, J=7.5 Hz, 1H), 7.20-7.14 (m, 1H), 7.10 (d, J=7.7 Hz, 1H), 5.07 (s, 2H), 3.43 (s, 3H), 3.20-3.05 (m, 3H), 2.55 (s, 3H), 2.21-2.11 (m, 1H), 1.43 (d, J=7.0 Hz, 3H), 1.22-1.15 (m, 2H), 1.08 (dt, J=8.2, 3.2 Hz, 2H); LCMS: C₂₃H₂₅N₅O requires: 387, found: m/z=388 [M+H]⁺.

### Example 373: 4-(2-fluoropropan-2-yl)-2-[3-[(2R)-1-(4-methyl-4H-1,2,4-triazol-3-yl)propan-2-yl]phenyl]-1H,2H,3H-pyrrolo[3,4-c]pyridin-1-one (373)

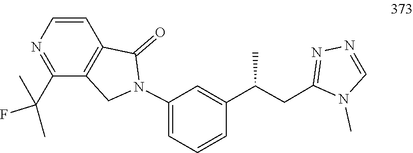 [SMILES: C[C@H](Cc1nncn1C)c1cccc(N2Cc3c(ccnc3C(C)(C)F)C2=O)c1]

Step 1: Synthesis of (R)-4-chloro-2-(3-(1-(4-methyl-4H-1,2,4-triazol-3-yl)propan-2-yl)phenyl)-2,3-dihydropyrrolo[3,4-c]pyridin-1-one. To a solution of methyl 3-(bromomethyl)-2-chloroisonicotinate (Example 258-1) (300 mg, 1.13 mmol) in EtOH (3 mL) were added (R)-3-(1-(4-methyl-4H-1,2,4-triazol-3-yl)propan-2-yl)aniline (245 mg, 1.13 mmol) and Et₃N (350 mg, 3.46 mmol). The mixture was stirred at 80° C. for 4 h. After cooled to rt, ethyl ether (10 mL) was added thereto. The solid was collected by filtration, washed with diethyl ether, dried to afford the title compound (360 mg, crude) as a yellow solid, which was used without purification. MS (ESI) calculated for (C₁₉H₁₈ClN₅O) [M+H]⁺, 368.1; found, 368.1.

Step 2: Synthesis of methyl 2-[3-[(2R)-1-(4-methyl-4H-1,2,4-triazol-3-yl)propan-2-yl]phenyl]-1-oxo-1H,2H,3H-pyrrolo[3,4-c]pyridine-4-carboxylate. To a solution of 4-chloro-2-[3-[(2R)-1-(4-methyl-4H-1,2,4-triazol-3-yl)propan-2-yl]phenyl]-1H,2H,3H-pyrrolo[3,4-c]pyridin-1-one (100 mg, 0.27 mmol) in methanol (10 mL) were added Pd(dppf)Cl₂ (20 mg, 0.03 mmol), triethylamine (556 mg, 5.49 mmol). The mixture was stirred at 70° C. for 12 h under CO (2 atm). The solids were filtered out. The filtrate was concentrated to afford the title compound (100 mg, crude) as a yellow oil, which was used without purification. MS (ESI) calculated for (C₂₁H₂₁N₅O₃) [M+H]⁺, 392.2; found, 392.0.

Step 3: Synthesis of 4-acetyl-2-[3-[(2R)-1-(4-methyl-4H-1,2,4-triazol-3-yl)propan-2-yl]phenyl]-1H,2H,3H-pyrrolo[3,4-c]pyridin-1-one. To a solution of methyl 2-[3-[(2R)-1-(4-methyl-4H-1,2,4-triazol-3-yl)propan-2-yl]phenyl]-1-oxo-1H,2H,3H-pyrrolo[3,4-c]pyridine-4-carboxylate (300 mg, 0.77 mmol) in THF (10 mL) was added chloro(methyl)magnesium (0.9 mL, 0.91 mmol) at −78° C. The mixture was stirred at −78° C. for 1 h. The mixture was quenched by the addition of water (30 mL) and the aqueous phase was extracted with EtOAc (100 mL×3). The combined organic layers were washed with brine, dried, filtered and concentrated. The residue was purified by flash column chromatography with 0-40% EtOAc in petroleum ether to afford the title compound (100 mg, 35%) as a colorless solid. MS (ESI) calculated for (C₂₁H₂₁N₅O₂) [M+H]⁺, 376.2; found, 376.0.

Step 4: Synthesis of 4-(2-hydroxypropan-2-yl)-2-[3-[(2R)-1-(4-methyl-4H-1,2,4-triazol-3-yl)propan-2-yl]phenyl]-1H,2H,3H-pyrrolo[3,4-c]pyridin-1-one. To a solution of 4-acetyl-2-[3-[(2R)-1-(4-methyl-4H-1,2,4-triazol-3-yl)propan-2-yl]phenyl]-1H,2H,3H-pyrrolo[3,4-c]pyridin-1-one (60 mg, 0.16 mmol) in THF (10 mL) was added bromo(methyl)magnesium (0.3 mL, 0.3 mmol). The solution was stirred at −78° C. for 1 h. The mixture was quenched by the addition of water (30 mL) and the aqueous phase was extracted with EtOAc (50 mL×3). The combined organic layers were washed with water, dried, filtered and concentrated. The residue was purified by prep-HPLC to afford the title compound (13.0 mg, 21%) as a colorless solid. MS (ESI) calculated for (C₂₂H₂₅N₅O₂) [M+H]⁺, 392.3; found, 392.0.

Step 5: Synthesis of 4-(2-fluoropropan-2-yl)-2-[3-[(2R)-1-(4-methyl-4H-1,2,4-triazol-3-yl)propan-2-yl]phenyl]-1H,2H,3H-pyrrolo[3,4-c]pyridin-1-one (373). To a solution of 4-(2-hydroxypropan-2-yl)-2-[3-[(2R)-1-(4-methyl-4H-1,2,4-triazol-3-yl)propan-2-yl]phenyl]-1H,2H,3H-pyrrolo[3,4-c]pyridin-1-one (10 mg, 0.03 mmol) in dichloromethane (10 mL) was added DAST (6.31 mg, 0.43 mmol). The solution was stirred at −78° C. for 1 h. The reaction was quenched by the addition of saturated NaHCO₃ aqueous. The aqueous phase was extracted with dichloromethane (20 mL×3). The combined organic layers were washed with brine, dried, filtered and concentrated. The residue was purified by prep-HPLC to the title compound (2.2 mg, 22%) as a colorless solid. ¹H NMR (400 MHz, Chloroform-d) δ 8.75 (d, J=4.8 Hz, 1H), 8.49 (s, 1H), 7.83 (s, 1H), 7.71 (d, J=4.8 Hz, 1H), 7.49 (d, J=8.0 Hz, 1H), 7.39-7.37 (m, 1H), 7.09-7.06 (m, 1H), 5.11 (s, 2H), 3.62-3.40 (m, 5H), 3.19-3.16 (m, 1H), 1.86 (s, 3H), 1.80 (s, 3H), 1.54 (d, J=6.8 Hz, 3H). MS (ESI) calculated for (C₂₂H₂₄FN₅O) [M+H]⁺, 394.2; found, 394.1.

### Example 374: (R)-6-(2-hydroxypropan-2-yl)-2-(3-(1-(4-methyl-4H-1,2,4-triazol-3-yl)propan-2-yl)phenyl)-4-(trifluoromethyl)isoindolin-1-one (374)

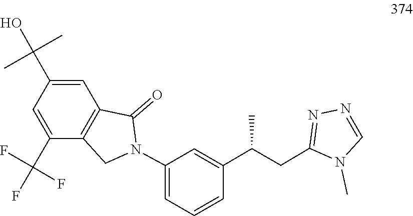 [SMILES: C[C@H](Cc1nncn1C)c1cccc(N2Cc3c(cc(C(C)(C)O)cc3C(F)(F)F)C2=O)c1]

Step 1: Synthesis of methyl 3-oxo-7-(trifluoromethyl)isoindoline-5-carboxylate. A mixture of 6-bromo-4-(trifluoromethyl)isoindolin-1-one (1.0 g, 3.58 mmol), Pd(dppf)Cl₂ (262.0 mg, 0.36 mmol) and triethylamine (1.09 g, 10.74 mmol) in methanol (20 mL) was stirred at 100° C. for 16 h under CO (20 atm). The reaction was then diluted with water, and the aqueous phase was extracted with EtOAc. The combined organic layers were washed with brine, dried, and filtered. The filtrate was concentrated and the residue was purified by reverse phase flash column chromatography with 0-10% methanol in dichloromethane to the title compound (600 mg, 64%) as a brown solid. MS (ESI) calculated for (C₁₁H₈F₃NO₃) [M+H]⁺, 260.0; found, 260.1.

Step 2: Synthesis of 6-(2-hydroxypropan-2-yl)-4-(trifluoromethyl)isoindolin-1-one. To a solution of methyl 3-oxo-7-(trifluoromethyl)isoindoline-5-carboxylate (500.0 mg, 1.93 mmol) in THF (10 mL) was added methylmagnesium bromide (1.93 mL, 3M) at 0° C. under N₂. The solution was stirred at 0° C. for 3 h. The reaction was then quenched by the addition of ammonium chloride and extracted with EtOAc. The combined organic layers were washed with brine, dried, and filtered. The filtrate was concentrated and the residue was purified by flash column chromatography with 0-10% methanol in dichloromethane to afford the title compound (100 mg, 20%) as a yellow solid. MS (ESI) calculated for (C₁₂H₁₂F₃NO₂) [M+H]⁺, 260.1; found, 260.2.

Step 3: Synthesis of (R)-6-(2-hydroxypropan-2-yl)-2-(3-(1-(4-methyl-4H-1,2,4-triazol-3-yl)propan-2-yl)phenyl)-4-(trifluoromethyl)isoindolin-1-one (374). To a degassed solution of 6-(2-hydroxypropan-2-yl)-4-(trifluoromethyl)isoindolin-1-one (100.0 mg, 0.39 mmol) in dioxane (5 mL) were added (R)-3-(2-(3-bromophenyl)propyl)-4-methyl-4H-1,2,4-triazole (108.1 mg, 0.39 mmol), Pd(OAc)₂ (17.3 mg, 0.08 mmol), XantPhos (89.3 mg, 0.15 mmol), Cs₂CO₃ (251.7 mg, 0.77 mmol). The solution was stirred at 100° C. for 16 h. The reaction was quenched by the addition of water, and then extracted with EtOAc. The combined organic layers were washed with brine, dried, and filtered. The filtrate was concentrated and the residue was purified by Prep-HPLC to the title compound (18.8 mg, 11%) as an off-white solid. 1H NMR (300 MHz, DMSO-d₆) δ 8.29 (s, 1H), 8.11 (d, J=8.7 Hz, 2H), 7.83-7.79 (m, 2H), 7.39-7.34 (m, 1H), 7.12 (d, J=7.5 Hz, 1H), 5.49 (s, 1H), 5.17 (s, 2H), 3.46 (s, 3H), 3.33-3.28 (m, 1H), 3.01 (d, J=7.5 Hz, 2H), 1.53 (s, 6H), 1.32 (d, J=6.9 Hz, 3H). MS (ESI) calculated for (C₂₄H₂₅F₃N₂) [M+H]⁺, 459.2; found, 458.8.

### Example 375: 2-[3-[(1 S,2S,3R)-2-methyl-3-(4-methyl-4H-1,2,4-triazol-3-yl)cyclopropyl]phenyl]-4-(trifluoromethyl)-2,3-dihydro-1H-isoindol-1-one (375)

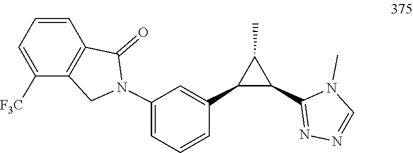 [SMILES: C[C@@H]1[C@@H](c2nncn2C)[C@H]1c1cccc(N2Cc3c(cccc3C(F)(F)F)C2=O)c1]

Step 1: Synthesis of ethyl (2R,3R and 2S,3S)-2-(3-bromophenyl)-3-methylcyclopropane-1-carboxylate. A mixture of l-bromo-3-[(1E)-prop-1-en-1-yl]benzene (McGuigan, C. et. al., Bioorg. Med. Chem., 2009, 17, 3025-3027) (17.1 g, 86.77 mmol) and CuCl (3.5 g, 35.35 mmol) in toluene (250 mL) was stirred at rt for 20 min under N₂, and then heated to 50° C. for 10 min. Ethyl 2-diazoacetate (49.0 g, 429.43 mmol) was added dropwise to the above mixture within 3.5 h at 50° C. The mixture was then stirred at 50° C. for 16 h before filtered. The filtrate was evaporated under vacuum. The residue was purified by flash column chromatography with 08% EtOAc in petroleum ether to afford the title compound (24.1 g, 98%) as a colorless oil.

Step 2: Synthesis of (2R,3R and 2S,3S)-2-(3-bromophenyl)-3-methylcyclopropane-1-carbohydrazide. A mixture of ethyl (2R,3R and 2S,3S)-2-(3-bromophenyl)-3-methylcyclopropane-1-carboxylate (24.1 g, 85.11 mmol) and hydrazine hydrate (100 mL, 80%) in EtOH (70 mL) was heated at 90° C. for 16 h. The mixture was evaporated under vacuum to afford racemic the title compound (25.4 g, crude) as a light yellow semi-solid, which was used without purification. MS (ESI) calculated for (C₁₁H₁₃BrN₂O) [M+H]⁺, 269.0, found, 268.8.

Step 3: Synthesis of (2R,3R and 2S,3S)-2-(3-bromophenyl)-3-methyl-N-[(methylcarbamothioyl)amino]cyclopropane-1-carboxamide. To a mixture of (2R,3R and 2S,3S)-2-(3-bromophenyl)-3-methylcyclopropane-1-carbohydrazide (25.4 g, 94.37 mmol) in THF (350 mL) was added isothiocyanatomethane (45.0 g, 615.51 mmol). The mixture was stirred at rt for 16 h. When the reaction was completed, the solvent was evaporated under vacuum. The residue was dissolved with diethyl ether (200 mL) and filtered. The solid was collected and dried under oven to afford the title compound (25.3 g, 78%) as a colorless solid. MS (ESI) calculated for (C₁₃H₁₆BrN₃OS) [M+H]⁺, 342.0, found, 341.9.

Step 4: Synthesis of 5-[(2R,3R and 2S,3S)-2-(3-bromophenyl)-3-methylcyclopropyl]-4-methyl-4H-1,2,4-triazole-3-thiol. A mixture of (2R,3R and 2S,3S)-2-(3-bromophenyl)-3-methyl-N-[(methylcarbamothioyl)amino]cyclopropane-1-carboxamide (21.8 g, 63.69 mmol) in NaOH (600 mL, 1 mol/L) was stirred at rt for 16 h. When the reaction was completed, the pH value of the mixture was adjusted to 4 with HCl (1 N). The mixture was extracted with dichloromethane. The combined organic layers were dried, and filtered. The filtrate was evaporated under vacuum to afford the title compound (17.0 g, crude) as a light yellow solid, which was used without purification. MS (ESI) calculated for (C₁₃H₁₄BrN₃S) [M+H]⁺, 324.0, found, 323.8.

Step 5: Synthesis of 3-[(2R,3R and 2S,3S)-2-(3-bromophenyl)-3-methylcyclopropyl]-4-methyl-4H-1,2,4-triazole. To a solution of 5-[(2R,3R and 2S,3S)-2-(3-bromophenyl)-3-methylcyclopropyl]-4-methyl-4H-1,2,4-triazole-3-thiol (17.0 g, 52.43 mmol) in CH₂Cl₂ (350 mL) was added HOAc (55 mL) and H₂O₂ (20.0 mL, 30%). The mixture was stirred at rt for 1.5 h. The mixture was then diluted with water (200 mL). The pH value of the mixture was adjusted to 7 with NaHCO₃ aqueous and extracted with dichloromethane. The combined organic layers were washed with brine, dried, and filtered. The filtrate was evaporated under vacuum. The residue was purified by flash column chromatography with 0˜5% methanol in dichloromethane, and then purified by reverse phase flash column chromatography with 45-55% acetonitrile in water to afford the title compound (13.0 g, 84%) as a brown oil. MS (ESI) calculated for (C₁₃H₁₄BrN₃) [M+H]⁺, 292.0, found, 291.8.

Step 6: Synthesis of 2-[3-[(2R,3R and 2S,3S)-2-methyl-3-(4-methyl-4H-1,2,4-triazol-3-yl)cyclopropyl]phenyl]-4-(trifluoromethyl)-2,3-dihydro-1H-isoindol-1-one. A degassed mixture of 3-[(2R,3R and 2S,3S)-2-(3-bromophenyl)-3-methylcyclopropyl]-4-methyl-4H-1,2,4-triazole (2.0 g, 6.85 mmol), 4-(trifluoromethyl)-2,3-dihydro-1H-isoindol-1-one (1.7 g, 7.81 mmol), Pd₂(dba)₃ (1.4 g, 1.49 mmol), XPhos (1.5 g, 3.15 mmol) and Cs₂CO₃ (6.7 g, 20.66 mmol) in 1,4-dioxane (70 mL) was heated at 100° C. for 16 h under N₂. The mixture was cooled to rt and then filtered. The filtrate was evaporated under vacuum. The residue was purified by flash column chromatography with 08% methanol in dichloromethane, and then purified by reverse phase flash column chromatography 5-50% acetonitrile in water (0.1% NH₄HCO₃) to afford the title compound (820.0 mg, 29%) as a light yellow solid. MS (ESI) calculated for (C₂₂H₁₉F₃N₄O) [M+H]⁺, 413.2, found, 412.9.

Step 7: Synthesis of 2-[3-[(1R,2R,3S and 1S,2S,3R)-2-methyl-3-(4-methyl-4H-1,2,4-triazol-3-yl)cyclopropyl]phenyl]-4-(trifluoromethyl)-2,3-dihydro-1H-isoindol-1-one (rac-375) and 2-[3-[((1R,2R,3R) and (1S,2S,3S))-2-methyl-3-(4-methyl-4H-1,2,4-triazol-3-yl)cyclopropyl]phenyl]-4-(trifluoromethyl)-2,3-dihydro-1H-isoindol-1-one (racemic iso-375). The mixture of 2-[3-[(2R,3R and 2S,3S)-2-methyl-3-(4-methyl-4H-1,2,4-triazol-3-yl)cyclopropyl]phenyl]-4-(trifluoromethyl)-2,3-dihydro-1H-isoindol-1-one (820.0 mg, 1.99 mmol) was separated by Prep-HPLC to afford:

2-[3-[(1R,2R,3S and 1S,2S,3R)-2-methyl-3-(4-methyl-4H-1,2,4-triazol-3-yl)cyclopropyl]phenyl]-4-(trifluoromethyl)-2,3-dihydro-1H-isoindol-1-one (rac-375): (80.0 mg, 9%, colorless solid) MS (ESI) calculated for (C₂₂H₉F₃N₄O) [M+H]⁺, 413.2, found, 412.9. ¹H NMR (400 MHz, DMSO-d₆) δ 8.14 (s, 1H), 8.08-8.03 (m, 2H), 7.81-7.72 (m, 2H), 7.48 (s, 1H), 7.17-7.13 (m, 1H), 6.71 (d, J=8.0 Hz, 1H), 5.12-4.99 (m, 2H), 3.38 (s, 3H), 2.43-2.40 (m, 1H), 2.33-2.25 (m, 2H), 1.37 (d, J=5.6 Hz, 3H).

2-[3-[(1R,2R,3R) and (1S,2S,3S)-2-methyl-3-(4-methyl-4H-1,2,4-triazol-3-yl)cyclopropyl]phenyl]-4-(trifluoromethyl)-2,3-dihydro-1H-isoindol-1-one (racemic iso-375): (310 mg, 37%, colorless solid) MS (ESI) calculated for (C₂₂H₁₉F₃N₄O) [M+H]⁺, 413.2, found, 412.9. ¹H NMR (400 MHz, DMSO-d₆) δ 8.40 (s, 1H), 8.11-8.04 (m, 2H), 7.82-7.78 (m, 3H), 7.41-7.37 (m, 1H), 7.08 (d, J=8.0 Hz, 1H), 5.25 (s, 2H), 3.68 (s, 3H), 2.58-2.55 (m, 1H), 2.50-2.48 (m, 1H), 1.80-1.75 (m, 1H), 1.02 (d, J=6.4 Hz, 3H).

Step 9: Synthesis of 2-[3-[(1R,2R,3S)-2-methyl-3-(4-methyl-4H-1,2,4-triazol-3-yl)cyclopropyl]phenyl]-4-(trifluoromethyl)-2,3-dihydro-1H-isoindol-1-one (ent-375) and 2-[3-[(1S,2S,3R)-2-methyl-3-(4-methyl-4H-1,2,4-triazol-3-yl)cyclopropyl]phenyl]-4-(trifluoromethyl)-2,3-dihydro-1H-isoindol-1-one (375). The racemic compound of 2-[3-[(1R,2R,3S and 1S,2S,3R)-2-methyl-3-(4-methyl-4H-1,2,4-triazol-3-yl)cyclopropyl]phenyl]-4-(trifluoromethyl)-2,3-dihydro-1H-isoindol-1-one (80.0 mg, 190.00 mmol) was separated by chiral-Prep-HPLC with the following conditions: [Column: CHIRALPAK IE, 2*25 cm, 5 um; Mobile Phase A:Hex-HPLC, Mobile Phase B: EtOH-HPLC; Flow rate: 15 mL/min; Gradient: 50 B to 50 B in 29 min; 220/254 nm] to afford:

2-[3-[(1R,2R,3S)-2-methyl-3-(4-methyl-4H-1,2,4-triazol-3-yl)cyclopropyl]phenyl]-4-(trifluoromethyl)-2,3-dihydro-1H-isoindol-1-one (ent-375): (38 mg, shorter retention time) MS (ESI) calculated for (C₂₂H₁₉F₃N₄O) [M+H]⁺, 413.2, found, 412.9. ¹H NMR (400 MHz, DMSO-d₆) δ 8.14 (s, 1H), 8.08-8.02 (m, 2H), 7.81-7.72 (m, 2H), 7.48 (s, 1H), 7.17-7.13 (m, 1H), 6.71 (d, J=8.0 Hz, 1H), 5.12-4.99 (m, 2H), 3.38 (s, 3H), 2.43-2.40 (m, 1H), 2.33-2.25 (m, 2H), 1.37 (d, J=5.6 Hz, 3H).

2-[3-[(1S,2S,3R)-2-methyl-3-(4-methyl-4H-1,2,4-triazol-3-yl)cyclopropyl]phenyl]-4-(trifluoromethyl)-2,3-dihydro-1H-isoindol-1-one (375): (25 mg, longer retention time) MS (ESI) calculated for (C₂₂H₁₉F₃N₄O) [M+H]⁺, 413.2, found, 412.9. ¹H NMR (300 MHz, DMSO-d₆) δ 8.16 (s, 1H), 8.08-8.02 (m, 2H), 7.81-7.72 (m, 2H), 7.48 (s, 1H), 7.17-7.12 (m, 1H), 6.71 (d, J=7.8 Hz, 1H), 5.14-4.98 (m, 2H), 3.38 (s, 3H), 2.44-2.39 (m, 1H), 2.34-2.24 (m, 2H), 1.37 (d, J=5.7 Hz, 3H).

### Example 376: (R)-6-cyclopropyl-N-(3-(1-(4-methyl-4H-1,2,4-triazol-3-yl)propan-2-yl)phenyl)-5-((methylamino)methyl)picolinamide (376)

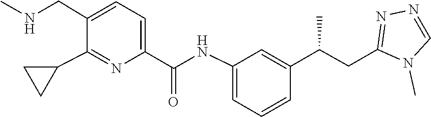 [SMILES: CNCc1ccc(C(=O)Nc2cccc([C@H](C)Cc3nncn3C)c2)nc1C1CC1]

The reductive amination was carried out in a similar fashion as for 447, step 4 using 6-cyclopropyl-5-formyl-N-{3-[(2R)-1-(4-methyl-4H-1,2,4-triazol-3-yl)propan-2-yl]phenyl}pyridine-2-carboxamide (20 mg, 0.05 mmol, 1 eq) and methylamine (7 mg, 0.21 mmol, 4 eq) as reactants afford title compound (14 mg, 67%) as colorless oil. ¹H NMR (500 MHz, Chloroform-d) δ 9.78 (s, 1H), 8.00 (d, J=7.8 Hz, 1H), 7.93 (s, 1H), 7.83 (d, J=7.9 Hz, 1H), 7.64 (s, 1H), 7.45 (d, J=8.4 Hz, 1H), 7.27 (m, 1H), 6.91 (d, J=1.1 Hz, 1H), 4.10 (s, 2H), 3.38 (q, J=7.1 Hz, 1H), 3.25 (s, 3H), 3.10 (dd, J=14.7, 6.8 Hz, 1H), 2.95 (dd, J=14.7, 7.7 Hz, 1H), 2.59 (s, 3H), 2.31-2.22 (m, 1H), 1.45 (d, J=7.0 Hz, 4H), 1.20-1.16 (m, 2H), 1.15-1.10 (m, 2H). LCMS: C₂₃H₂₈N₆O requires: 404, found: m/z=405 [M+H]⁺.

### Example 377: 6-(4-methyl-1H-pyrazol-3-yl)-2-{3-[(2R)-1-(4-methyl-4H-1,2,4-triazol-3-yl)propan-2-yl]phenyl}-4-(trifluoromethyl)-2,3-dihydro-1H-isoindol-1-one (377)

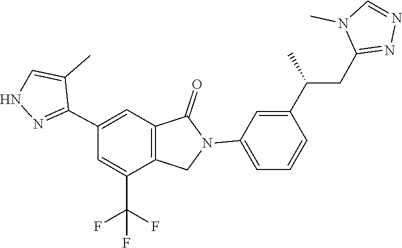 [SMILES: Cc1c[nH]nc1-c1cc2c(c(C(F)(F)F)c1)CN(c1cccc([C@H](C)Cc3nncn3C)c1)C2=O]

To a degassed solution of 6-bromo-2-{3-[(2R)-1-(4-methyl-4H-1,2,4-triazol-3-yl)propan-2-yl]phenyl}-4-(trifluoromethyl)-2,3-dihydro-1H-isoindol-1-one (550 mg, 1.15 mmol) in dioxane (10 mL) and water (2 mL) were added tert-butyl 4-methyl-3-(4,4,5,5-tetramethyl-1,3,2-dioxaborolan-2-yl)-1H-pyrazole-1-carboxylate (707 mg, 2.30 mmol), disodium carbonate (364 mg, 3.44 mmol), and Pd(dppf)Cl₂ (93 mg, 0.11 mmol). The mixture was stirred at 120° C. for 16 h. The mixture was diluted with water and 15% IPA/chloroform. The organic layer was separated, dried, filtered and concentrated. The residue was purified by HPLC. The solution was partitioned between 15% IPA/chloroform and saturated sodium bicarbonate. The organic layer was dried, filtered, and concentrated to give the title compound (250 mg, 45%): ¹H NMR (500 MHz, DMSO-d₆) δ 12.96 (s, 1H), 8.27 (d, J=18.8 Hz, 3H), 7.92-7.78 (m, 2H), 7.72 (s, 1H), 7.38 (t, J=7.8 Hz, 1H), 7.13 (d, J=7.5 Hz, 1H), 5.24 (s, 2H), 3.45 (s, 3H), 3.34 (d, J=7.1 Hz, 1H), 3.18 (d, J=5.2 Hz, 3H), 3.03 (dd, J=7.4, 2.6 Hz, 2H), 1.33 (d, J=6.9 Hz, 3H); LCMS: C₂₅H₂₃F₃N₆O requires: 480, found: m/z=481 [M+H]⁺.

### Example 378: 6-(3,5-dimethyl-1H-pyrazol-4-yl)-2-{3-[(2R)-1-(4-methyl-4H-1,2,4-triazol-3-yl)propan-2-yl]phenyl}-4-(trifluoromethyl)-2,3-dihydro-1H-isoindol-1-one (378)

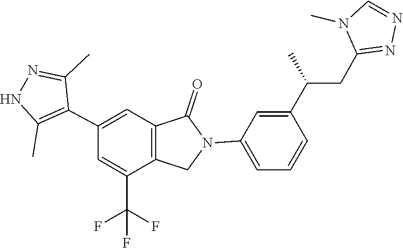 [SMILES: Cc1n[nH]c(C)c1-c1cc2c(c(C(F)(F)F)c1)CN(c1cccc([C@H](C)Cc3nncn3C)c1)C2=O]

The coupling reaction was carried out in a similar fashion as for 377 using 6-bromo-2-{3-[(2R)-1-(4-methyl-4H-1,2,4-triazol-3-yl)propan-2-yl]phenyl}-4-(trifluoromethyl)-2,3-dihydro-1H-isoindol-1-one (500 mg, 1.04 mmol, 1.0 eq.) and tert-butyl 3,5-dimethyl-4-(4,4,5,5-tetramethyl-1,3,2-dioxaborolan-2-yl)-1H-pyrazole-1-carboxylate (670 mg, 2.09 mmol, 2.0 eq.) as reactants to afford the title compound (145 mg, 28%) as an off-white solid: ¹H NMR (500 MHz, DMSO-d6) δ 12.52 (s, 1H), 8.29 (s, 1H), 8.01-7.68 (m, 4H), 7.38 (t, J=7.9 Hz, 1H), 7.24-7.03 (m, 1H), 5.22 (s, 2H), 3.47 (s, 3H), 3.38-3.29 (m, 2H), 3.13-2.90 (m, 1H), 2.26 (s, 6H), 1.33 (d, J=6.9 Hz, 3H); LCMS: C₂₆H₂₅F₃N₆O requires: 494, found: m/z=495 [M+H]⁺.

### Example 379: 2-(3-((R)-1-(4-methyl-4H-1,2,4-triazol-3-yl)propan-2-yl)phenyl)-6-(((S)-pyrrolidin-3-yl)amino)-4-(trifluoromethyl)isoindolin-1-one (379)

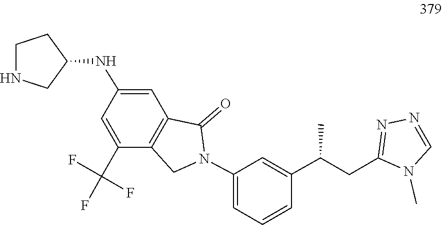 [SMILES: C[C@H](Cc1nncn1C)c1cccc(N2Cc3c(cc(N[C@H]4CCNC4)cc3C(F)(F)F)C2=O)c1]

Step 1: Synthesis of tert-butyl (S)-3-((2-(3-((R)-1-(4-methyl-4H-1,2,4-triazol-3-yl)propan-2-yl)phenyl)-3-oxo-7-(trifluoromethyl)isoindolin-5-yl)amino)pyrrolidine-1-carboxylate. A degassed solution of (R)-6-bromo-2-(3-(1-(4-methyl-4H-1,2,4-triazol-3-yl)propan-2-yl)phenyl)-4-(trifluoromethyl)isoindolin-1-one (180 mg, 0.38 mmol), tert-butyl (S)-3-aminopyrrolidine-1-carboxylate (70 mg, 0.38 mmol), XPhos (18 mg, 0.04 mmol), XPhos Pd G₃ (32 mg, 0.04 mmol) and Cs₂CO₃ (245 mg, 0.75 mmol) in dioxane (4 mL) was stirred at 90° C. for 16 h under nitrogen. When the reaction was completed, the reaction was quenched by the addition of water (20 mL). The aqueous solution was extracted with EtOAc (20 mL×3). The combined organic solution was dried, and concentrated.the residue, which was purified by flash column chromatography with 0˜5% methanol in dichloromethane to afford the title compound (170 mg, ˜70% purity) as a yellow oil, which was used without purification. MS (ESI) calculated for (C₃₀H₃₅F₃N₆O₃) [M+H]⁺, 585.3; found, 585.3.

Step 2: Synthesis of 2-(3-((R)-1-(4-methyl-4H-1,2,4-triazol-3-yl)propan-2-yl)phenyl)-6-(((S)-pyrrolidin-3-yl)amino)-4-(trifluoromethyl)isoindolin-1-one formate (379). To a stirred solution of tert-butyl (S)-3-((2-(3-((R)-1-(4-methyl-4H-1,2,4-triazol-3-yl)propan-2-yl)phenyl)-3-oxo-7-(trifluoromethyl)isoindolin-5-yl)amino)pyrrolidine-1-carboxylate (170 mg, 0.20 mmol, 70% purity) in dichloromethane (5 mL) was added TFA (1 mL). The solution was stirred at rt for 2 h. When the reaction was completed, the solution was concentrated to give the residue, which was purified by Prep-HPLC to the title compound (54.4 mg, 51%) as a colorless solid. MS (ESI) calculated for (C₂₅H₂₇F₃N₆O) [M+H]⁺, 485.2; found, 485.3. ¹H NMR (400 MHz, DMSO-d₆) δ 8.28 (s, 1H), 7.81-7.73 (m, 2H), 7.38-7.31 (m, 1H), 7.18 (d, J=2.0 Hz, 1H), 7.15-7.05 (m, 2H), 6.87 (d, J=6.4 Hz, 1H), 5.00 (s, 2H), 4.27-4.16 (m, 1H), 3.45 (s, 3H), (3.41-3.34 (m, 1H), 3.34-3.26 (m, 1H), 3.25-3.12 (m, 2H), 3.03-2.93 (m, 3H), 2.27-2.14 (m, 1H), 1.89-1.74 (m, 1H), 1.31 (d, J=6.9 Hz, 3H).

### Example 380: 6-(methyl((S)-pyrrolidin-3-yl)amino)-2-(3-((R)-1-(4-methyl-4H-1,2,4-triazol-3-yl)propan-2-yl)phenyl)-4-(trifluoromethyl)isoindolin-1-one (380)

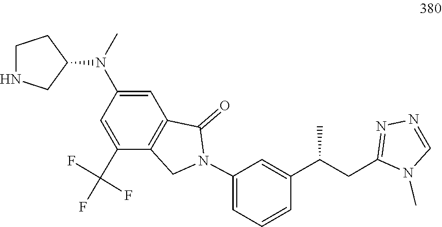 [SMILES: C[C@H](Cc1nncn1C)c1cccc(N2Cc3c(cc(N(C)[C@H]4CCNC4)cc3C(F)(F)F)C2=O)c1]

Step 1: Synthesis of tert-butyl (S)-3-(methyl(2-(3-((R)-1-(4-methyl-4H-1,2,4-triazol-3-yl)propan-2-yl)phenyl)-3-oxo-7-(trifluoromethyl)isoindolin-5-yl)amino)pyrrolidine-1-carboxylate. A degassed mixture of (R)-6-bromo-2-(3-(1-(4-methyl-4H-1,2,4-triazol-3-yl)propan-2-yl)phenyl)-4-(trifluoromethyl)isoindolin-1-one (200 mg, 0.42 mmol), tert-butyl (S)-3-(methylamino)pyrrolidine-1-carboxylate (84 mg, 0.42 mmol), XPhos (20 mg, 0.04 mmol), XPhos Pd G₃ (35 mg, 0.04 mmol) and Cs₂CO₃ (273 mg, 0.84 mmol) in dioxane (4 mL) was stirred at 90° C. for 16 h under nitrogen. When the reaction was completed, the reaction was quenched by the addition of 20 mL water. The aqueous solution was extracted with EtOAc (20 mL×3). The combined organic solution was dried, and concentrated to give the residue, which was purified by chromatography B to afford the title compound (129 mg, 65% purity) as a yellow oil, which was used without purification. MS (ESI) calculated for (C₃₁H₃₇F₃N₆O₃) [M+H]⁺, 599.3; found, 599.3.

Step 2: Synthesis of 6-(methyl((S)-pyrrolidin-3-yl)amino)-2-(3-((R)-1-(4-methyl-4H-1,2,4-triazol-3-yl)propan-2-yl)phenyl)-4-(trifluoromethyl)isoindolin-1-one formate (380). To a stirred solution of tert-butyl (S)-3-(methyl(2-(3-((R)-1-(4-methyl-4H-1,2,4-triazol-3-yl)propan-2-yl)phenyl)-3-oxo-7-(trifluoromethyl)isoindolin-5-yl)amino)pyrrolidine-1-carboxylate (129 mg, 0.14 mmol, 65% purity) in dichloromethane (5 mL) was added TFA (1 mL). The solution was stirred at rt for 2 h. When the reaction was completed, the solution was concentrated to give the residue, which was purified by Prep-HPLC to afford the title compound (27.4 mg, 36%) as a light yellow solid. MS (ESI) calculated for (C₂₆H₂₉F₃N₆O) [M+H]⁺, 499.2; found, 499.2. ¹H NMR (400 MHz, Methanol-d₄) δ 8.54 (s, 1H), 8.27 (s, 1H), 7.74-7.65 (m, 2H), 7.56-7.50 (m, 1H), 7.47-7.43 (m, 1H), 7.42-7.35 (m, 1H), 7.17-7.09 (m, 1H), 5.02 (s, 2H), 4.89-4.86 (m, 1H), 3.63-3.50 (m, 2H), 3.47 (s, 3H), 3.42-3.34 (m, 2H), 3.30-3.24 (m, 1H), 3.21-3.07 (m, 2H), 3.01 (s, 3H), 2.40-2.29 (m, 1H), 2.26-2.14 (m, 1H), 1.45 (d, J=6.8 Hz, 3H).

### Example 381: 6-(5-methyl-1H-pyrazol-4-yl)-2-{3-[(2R)-1-(4-methyl-4H-1,2,4-triazol-3-yl)propan-2-yl]phenyl}-4-(trifluoromethyl)-2,3-dihydro-1H-isoindol-1-one (381)

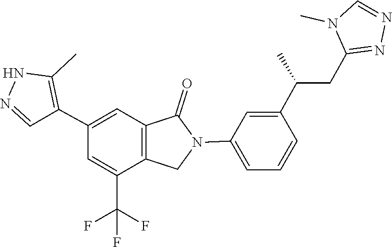 [SMILES: Cc1[nH]ncc1-c1cc2c(c(C(F)(F)F)c1)CN(c1cccc([C@H](C)Cc3nncn3C)c1)C2=O]

The coupling reaction was carried out in a similar fashion as for 377 using 6-bromo-2-{3-[(2R)-1-(4-methyl-4H-1,2,4-triazol-3-yl)propan-2-yl]phenyl}-4-(trifluoromethyl)-2,3-dihydro-1H-isoindol-1-one (500 mg, 1.04 mmol, 1.0 eq.) and tert-butyl 3-methyl-4-(4,4,5,5-tetramethyl-1,3,2-dioxaborolan-2-yl)-1H-pyrazole-1-carboxylate (640 mg, 2.09 mmol, 2.0 eq.) as reactants afford the title compound (250 mg, 50%) as an off-white solid: 1H NMR (500 MHz, DMSO-d6) δ 12.86 (d, J=31.9 Hz, 1H), 8.29 (s, 1H), 8.05 (d, J=13.0 Hz, 2H), 7.82 (dt, J=7.3, 1.8 Hz, 2H), 7.46-7.32 (m, 1H), 7.13 (dt, J=7.6, 1.3 Hz, 1H), 5.20 (s, 2H), 3.46 (d, J=4.5 Hz, 3H), 3.32 (d, J=4.8 Hz, 1H), 3.12-2.95 (m, 2H), 2.46 (s, 3H), 1.33 (d, J=6.9 Hz, 3H); LCMS: C₂₅H₂₃F₃N₆O requires: 480, found: m/z=481 [M+H]⁺.

### Example 382 and 383: 2-(3-((R)-1-(4-methyl-4H-1,2,4-triazol-3-yl)propan-2-yl)phenyl)-6-((S)-piperidin-3-yl)-4-(trifluoromethyl)isoindolin-1-one (382) and 2-(3-((R)-1-(4-methyl-4H-1,2,4-triazol-3-yl)propan-2-yl)phenyl)-6-((R)-piperidin-3-yl)-4-(trifluoromethyl)isoindolin-1-one (383)

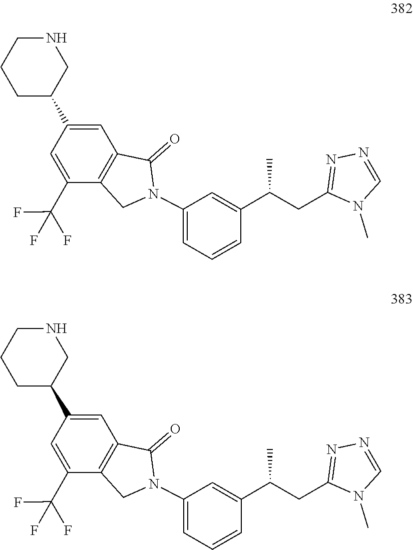 [SMILES: C[C@H](Cc1nncn1C)c1cccc(N2Cc3c(cc([C@@H]4CCCNC4)cc3C(F)(F)F)C2=O)c1 ; C[C@H](Cc1nncn1C)c1cccc(N2Cc3c(cc([C@H]4CCCNC4)cc3C(F)(F)F)C2=O)c1]

Step 1: Synthesis of 6-bromo-4-(trifluoromethyl)-2,3-dihydro-1H-isoindol-1-one. To a stirred solution of methyl 5-bromo-2-(bromomethyl)-3-(trifluoromethyl)benzoate (Example L & Example M, Step 2) (11.2 g, 29.79 mmol) in THF (50 mL) was added NH₃ (7M in methanol, 50 mL). The mixture was stirred at rt for 16 h before concentrated. The residue was diluted with water and extracted with EtOAc for three times. The organic layers were washed with brine, dried, filtered and concentrated. The residue was purified by flash column chromatography with 0-30% EtOAc in petroleum ether to afford the title compound (8.1 g, 53%) as a colorless solid. MS (ESI) calculated for (C₉H₅BrF₃NO) [M+H]⁺, 280.0; found, 280.1.

Step 2: Synthesis of tert-butyl 5-(3-oxo-7-(trifluoromethyl)isoindolin-5-yl)-3,6-dihydropyridine-1(2H)-carboxylate. To a degassed solution of 6-bromo-4-(trifluoromethyl)-2,3-dihydro-1H-isoindol-1-one (1.0 g, 3.57 mmol) in toluene (30 mL) and ethanol (15 mL) were added tert-butyl 5-(4,4,5,5-tetramethyl-1,3,2-dioxaborolan-2-yl)-1,2,3,6-tetrahydropyridine-1-carboxylate (1.3 g, 4.30 mmol), Na₂CO₃ (3.0 g, 28.68 mmol) and Pd(PPh₃)₄ (290 mg, 0.25 mmol). The mixture was stirred at 80° C. for 16 h under nitrogen. When the reaction was completed, the reaction was quenched by the addition of water (50 mL). The aqueous solution was extracted with EtOAc (30 mL×3). The combined organic solution was dried, and concentrated to give the residue, which was purified by reverse phase flash column chromatography with 5-50% acetonitrile in water to afford the title compound (900 mg, 66%) as a light yellow solid. MS (ESI) calculated for (C₁₉H₂₁F₃N₂O₃) [M+H]⁺, 383.2; found, 383.2.

Step 3: Synthesis of tert-butyl 3-(3-oxo-7-(trifluoromethyl)isoindolin-5-yl)piperidine-1-carboxylate. To a solution of tert-butyl 5-(3-oxo-7-(trifluoromethyl)isoindolin-5-yl)-3,6-dihydropyridine-1(2H)-carboxylate (900 mg, 2.36 mmol) in methanol (20 mL) was added Pd/C (400 mg). The mixture was stirred at 20° C. for 16 h under hydrogen. When the reaction was completed, the solids were filtered out. The filtrate was concentrated to give the residue, which was purified by reverse phase flash column chromatography with 5-50% acetonitrile in water to the title compound (550 mg, 61%) as a light yellow solid. MS (ESI) calculated for (C₁₉H₂₃F₃N₂O₃) [M+H]⁺, 385.2; found, 385.2.

Step 4: Synthesis of tert-butyl 3-(2-(3-((R)-1-(4-methyl-4H-1,2,4-triazol-3-yl)propan-2-yl)phenyl)-3-oxo-7-(trifluoromethyl)isoindolin-5-yl)piperidine-1-carboxylate. A degassed mixture of tert-butyl 3-(3-oxo-7-(trifluoromethyl)isoindolin-5-yl)piperidine-1-carboxylate (120 mg, 0.31 mmol), (R)-3-(2-(3-bromophenyl)propyl)-4-methyl-4H-1,2,4-triazole (87 mg, 0.31 mmol), XPhos Pd G3 (26 mg, 0.03 mmol), XPhos (15 mg, 0.03 mmol) and Cs₂CO₃ (204 mg, 0.62 mmol) in dioxane (5 mL) was stirred at 90° C. for 4 h under nitrogen. When the reaction was completed, the reaction was quenched by the addition of water (20 mL). The aqueous solution was extracted with EtOAc (20 mL×3). The combined organic layers were dried, and concentrated to give the residue, which was purified by flash column chromatography with 0-10% methanol in dichloromethane to the title compound (60 mg, 33%) as a light yellow oil. MS (ESI) calculated for (C₃₁H₃₆F₃N₅O₃) [M+H]⁺, 584.3; found, 584.3.

Step 5: Synthesis of 2-(3-((R)-1-(4-methyl-4H-1,2,4-triazol-3-yl)propan-2-yl)phenyl)-6-((R)-piperidin-3-yl)-4-(trifluoromethyl)isoindolin-1-one (382) and 2-(3-((R)-1-(4-methyl-4H-1,2,4-triazol-3-yl)propan-2-yl)phenyl)-6-((S)-piperidin-3-yl)-4-(trifluoromethyl)isoindolin-1-one (383). To a stirred solution of tert-butyl 3-(2-(3-((R)-1-(4-methyl-4H-1,2,4-triazol-3-yl)propan-2-yl)phenyl)-3-oxo-7-(trifluoromethyl)isoindolin-5-yl)piperidine-1-carboxylate (60 mg, 0.10 mmol) in dichloromethane (4 mL) was added TFA (1 mL). The solution was stirred at 10° C. for 1.5 h The solution was concentrated to give the residue, which was purified by Prep-HPLC with the following conditions: [Column: XBridge Prep OBD C18 Column 30×150 mm 5 um; Mobile Phase A: Water(10 mmol/L NH₄HCO₃), Mobile Phase B: ACN; Flow rate: 60 mL/min; Gradient: 18% B to 39% B in 7 min; 254/220 nm] to afford the diastereomeric mixture of 2-(3-((R)-1-(4-methyl-4H-1,2,4-triazol-3-yl)propan-2-yl)phenyl)-6-(piperidin-3-yl)-4-(trifluoromethyl)isoindolin-1-one (32.6 mg, 66%) as colorless solid. The diastereomeric mixture was separated by prep-Chiral-HPLC with the following conditions: [Column: CHIRALPAK IG, 20*250 mm, 5 um; Mobile Phase A: Hex: methylene chloride=1:1(10 mM NH₃-MEOH)-, Mobile Phase B: EtOH-; Flow rate: 18 mL/min; Gradient: 50 B to 50 B in 14 min; 220/254 nm] to afford:

2-(3-((R)-1-(4-methyl-4H-1,2,4-triazol-3-yl)propan-2-yl)phenyl)-6-((R)-piperidin-3-yl)-4-(trifluoromethyl)isoindolin-1-one (382): (7.9 mg, colorless solid, shorter retention time) MS (ESI) calculated for (C₂₆H₂₈F₃N₅O) [M+H]⁺, 484.2; found, 484.2. ¹H NMR (400 MHz, DMSO-d₆) δ 8.28 (s, 1H), 7.93 (s, 1H), 7.89 (s, 1H), 7.83-7.75 (m, 2H), 7.36 (t, J=7.6 Hz, 1H), 7.11 (d, J=7.6 Hz, 1H), 5.15 (s, 2H), 3.45 (s, 3H), 3.08-2.86 (m, 5H), 2.71-2.64 (m, 1H), 2.62-2.53 (m, 1H), 1.99-1.89 (m, 1H), 1.74-1.64 (m, 2H), 1.58-1.47 (m, 1H), 1.31 (d, J=6.8 Hz, 3H).

2-(3-((R)-1-(4-methyl-4H-1,2,4-triazol-3-yl)propan-2-yl)phenyl)-6-((S)-piperidin-3-yl)-4-(trifluoromethyl)isoindolin-1-one (383): (10.4 mg, yellow solid, longer retention time)MS (ESI) calculated for (C₂₆H₂₈F₃N₅O) [M+H]⁺, 484.2; found, 484.2. ¹H NMR (400 MHz, DMSO-d₆) δ 8.28 (s, 1H), 8.00 (s, 1H), 7.95 (s, 1H), 7.83-7.76 (m, 2H), 7.37 (t, J=7.6 Hz, 1H), 7.12 (d, J=7.6 Hz, 1H), 5.17 (s, 2H), 3.46 (s, 3H), 3.41-3.06 (m, 5H), 3.06-2.98 (m, 3H), 2.83-2.74 (m, 1H), 1.99-1.91 (m, 1H), 1.85-1.71 (m, 2H), 1.31 (d, J=6.8 Hz, 3H).

### Example 384: (R)-2-(3-(1-(4-methyl-4H-1,2,4-triazol-3-yl)propan-2-yl)phenyl)-6-(1H-pyrazol-1-yl)-4-(trifluoromethyl)isoindolin-1-one (384)

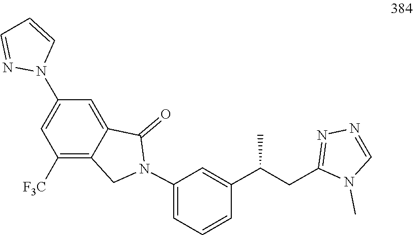 [SMILES: C[C@H](Cc1nncn1C)c1cccc(N2Cc3c(cc(-n4cccn4)cc3C(F)(F)F)C2=O)c1]

Step 1: Synthesis of 6-bromo-2-[3-[(2R)-1-(4-methyl-4H-1,2,4-triazol-3-yl)propan-2-yl]phenyl]-4-(trifluoromethyl)-2,3-dihydro-1H-isoindol-1-one. To a solution of methyl 5-bromo-2-(bromomethyl)-3-(trifluoromethyl)benzoate (15 g, 39.90 mmol) in methanol (150 mL) were added 3-[(2R)-1-(4-methyl-4H-1,2,4-triazol-3-yl)propan-2-yl]aniline (8.7 g, 40.22 mmol) and AgNO₃ (7.45 g, 44.08 mmol) at rt. The mixture was stirred at 50° C. for 12 h. The mixture was diluted with water (120 mL) and extracted with EtOAc (60 mL×2). The combined organic layers were dried, filtered and concentrated. The residue was purified by flash column chromatography with 0-10% methanol in dichloromethane to afford the title compound (16 g, 80%) as a light yellow solid. MS (ESI) calculated for (C₂₁H₁₈BrF₃N₄O) [M+H]⁺, 479.1; found, 479.0.

Step 2: Synthesis of (R)-2-(3-(1-(4-methyl-4H-1,2,4-triazol-3-yl)propan-2-yl)phenyl)-6-(1H-pyrazol-1-yl)-4-(trifluoromethyl)isoindolin-1-one (384). A degassed mixture of (R)-6-bromo-2-(3-(1-(4-methyl-4H-1,2,4-triazol-3-yl)propan-2-yl)phenyl)-4-(trifluoromethyl)isoindolin-1-one (350 mg, 0.73 mmol), 1H-pyrazole (75 mg, 1.10 mmol), N¹,N²-dimethylethane-1,2-diamine (13 mg, 0.15 mmol), CuI (14 mg, 0.07 mmol) and Cs₂CO₃ (477 mg, 1.46 mmol) in dioxane (5 mL) was stirred at 120° C. for 16 h under nitrogen. The reaction was diluted with water (20 mL). and extracted with EtOAc (20 mL×3). The combined organic solution was dried, filtered and concentrated to give the residue, which was purified by chromatography B then purified by Prep-HPLC with the following conditions: [(Column: SunFire Prep C18 OBD Column 19×150 mm 5 um 10 nm; Mobile Phase A: Water(0.1% FA), Mobile Phase B: ACN; Flow rate: 25 mL/min; Gradient: 32% B to 45% B in 10 min; 254/220 nm] to the title compound (30.6 mg, 9%) as a light yellow solid. MS (ESI) calculated for (C₂₄H₂₁F₃N₆O) [M+H]⁺, 467.2; found, 467.1. ¹H NMR (400 MHz, DMSO-d₆) δ 8.90 (d, J=2.4 Hz, 1H), 8.53-8.46 (m, 2H), 8.28 (s, 1H), 7.87 (d, J=1.6 Hz, 1H), 7.85-7.78 (m, 2H), 7.42-7.34 (m, 1H), 7.17-7.10 (m, 1H), 6.65 (d, J=2.4 Hz, 1H), 5.23 (s, 2H), 3.46 (s, 3H), 3.38-3.28 (m, 1H), 3.02 (d, J=7.2 Hz, 2H), 1.33 (d, J=6.8 Hz, 3H).

### Example 385: 6-(3,5-dimethyl-1H-pyrazol-4-yl)-2-(3-{3-[(4-methyl-4H-1,2,4-triazol-3-yl)methyl]oxetan-3-yl}phenyl)-4-(trifluoromethyl)-2,3-dihydro-1H-isoindol-1-one (385)

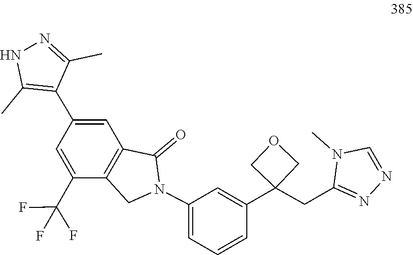 [SMILES: Cc1n[nH]c(C)c1-c1cc2c(c(C(F)(F)F)c1)CN(c1cccc(C3(Cc4nncn4C)COC3)c1)C2=O]

The coupling reaction was carried out in a similar fashion as for 377 using 6-bromo-2-(3-(3-((4-methyl-4H-1,2,4-triazol-3-yl)methyl)oxetan-3-yl)phenyl)-4-(trifluoromethyl)isoindolin-1-one (330 mg, 0.65 mmol, 1.0 eq.) and tert-butyl 3,5-dimethyl-4-(4,4,5,5-tetramethyl-1,3,2-dioxaborolan-2-yl)-1H-pyrazole-1-carboxylate (420 mg, 1.30 mmol, 2.0 eq.) as reactants afford the title compound (120 mg, 35%) as an off-white solid: ¹H NMR (500 MHz, DMSO-d6) δ 12.53 (s, 1H), 8.20 (s, 2H), 7.98-7.80 (m, 3H), 7.45 (t, J=2.0 Hz, 1H), 7.37 (t, J=7.9 Hz, 1H), 6.78 (dt, J=7.7, 1.2 Hz, 1H), 5.19-5.07 (m, 2H), 4.98 (d, J=6.0 Hz, 2H), 4.90 (d, J=6.1 Hz, 2H), 3.52 (s, 2H), 2.91 (s, 3H), 2.26 (d, J=26.3 Hz, 5H); LCMS: C₂₇H₂₅F₃N₅O₂ requires: 522, found: m/z=523 [M+H]⁺.

### Example 386: 2-(3-(3-((4-Methyl-4H-1,2,4-triazol-3-yl)methyl)oxetan-3-yl)phenyl)-6-(3-methyl-3,8-diazabicyclo[3.2.1]octan-8-yl)-4-(trifluoromethyl)isoindolin-1-one (386)

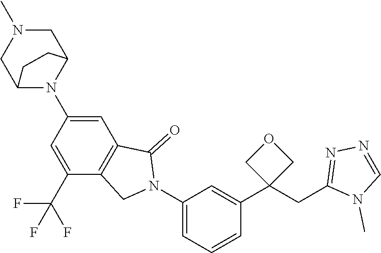 [SMILES: CN1CC2CCC(C1)N2c1cc2c(c(C(F)(F)F)c1)CN(c1cccc(C3(Cc4nncn4C)COC3)c1)C2=O]

A solution of 6-bromo-2-(3-(3-((4-methyl-4H-1,2,4-triazol-3-yl)methyl)oxetan-3-yl)phenyl)-4-(trifluoromethyl)isoindolin-1-one (785 mg, 1.6 mmol, 1.0 eq.), 3-methyl-3,8-diazabicyclo[3.2.1]octane dihydrochloride (370 mg, 1.9 mmol, 1.2 eq.), palladium acetate (69 mg, 0.31 mmol, 0.2 eq.), [5-(diphenylphosphanyl)-9,9-dimethyl-9H-xanthen-4-yl]diphenylphosphane (360 mg, 0.62 mmol, 0.4 eq.) and cesium carbonate (2.5 g, 7.7 mmol, 5 eq.) in 1,4-dioxane (6.5 mL) was stirred at 95° C. for 16 h under nitrogen atmosphere. The mixture was filtered and concentrated to dryness. The residue was purified by prep-HPLC to afford the title compound (4.5 mg, 12%) as an off-white solid: ¹H NMR (500 MHz, DMSO-d₆) δ 8.17 (s, 1H), 7.89 (dd, J=7.9, 2.1 Hz, 1H), 7.39-7.28 (m, 4H), 6.79-6.68 (m, 1H), 5.00-4.77 (m, 6H), 4.46 (s, 2H), 3.49 (s, 3H), 3.37-3.22 (m, 2H), 2.88 (s, 2H), 2.23 (d, J=10.9 Hz, 2H), 2.06 (s, 3H), 1.99-1.86 (m, 4H); LCMS: C₂₉H₃₁F₃N₆O₂ requires: 552, found: m/z=553 [M+H]⁺.

### Example 387: 2-(3-(3-((4-Methyl-4H-1,2,4-triazol-3-yl)methyl)oxetan-3-yl)phenyl)-6-(5-methyl-2,5-diazabicyclo[2.2.2]octan-2-yl)-4-(trifluoromethyl)isoindolin-1-one (387)

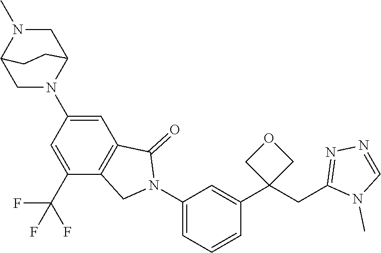 [SMILES: CN1CC2CCC1CN2c1cc2c(c(C(F)(F)F)c1)CN(c1cccc(C3(Cc4nncn4C)COC3)c1)C2=O]

The C—N coupling reaction with potassium phosphate was carried out in a similar fashion as for 386 using 6-bromo-2-(3-(3-((4-methyl-4H-1,2,4-triazol-3-yl)methyl)oxetan-3-yl)phenyl)-4-(trifluoromethyl)isoindolin-1-one (50 mg, 0.10 mmol, 1 eq.) and 2-methyl-2,5-diazabicyclo[2.2.2]octane dihydrochloride (24 mg, 0.12 mmol, 1.2 eq.) as reactants afforded the title compound (13 mg, 23%) as an off-white solid: ¹H NMR (500 MHz, DMSO-d₆) δ 8.12 (s, 1H), 7.82 (dd, J=8.0, 2.1 Hz, 1H), 7.30-7.23 (m, 2H), 7.09 (s, 2H), 6.70-6.65 (m, 1H), 4.94-4.75 (m, 6H), 4.04 (s, 1H), 3.64 (d, J=9.9 Hz, 1H), 3.44 (s, 2H), 3.27-3.21 (m, 1H), 3.20-3.15 (m, 1H), 2.83 (s, 3H), 2.78-2.69 (m, 2H), 2.26 (s, 3H), 1.95 (s, 1H), 1.73 (dd, J=24.3, 11.9 Hz, 2H), 1.54-1.48 (m, 1H); LCMS: C₂₉H₃₁F₃N₆O₂ requires: 552, found: m/z=553 [M+H]⁺.

### Example 388: 2-(3-(3-((4-Methyl-4H-1,2,4-triazol-3-yl)methyl)oxetan-3-yl)phenyl)-6-(8-methyl-3,8-diazabicyclo[3.2.1]octan-3-yl)-4-(trifluoromethyl)isoindolin-1-one (388)

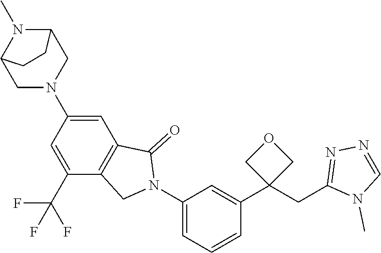 [SMILES: CN1C2CCC1CN(c1cc3c(c(C(F)(F)F)c1)CN(c1cccc(C4(Cc5nncn5C)COC4)c1)C3=O)C2]

The C—N coupling reaction with potassium phosphate was carried out in a similar fashion as for 386 using 6-bromo-2-(3-(3-((4-methyl-4H-1,2,4-triazol-3-yl)methyl)oxetan-3-yl)phenyl)-4-(trifluoromethyl)isoindolin-1-one (50 mg, 0.10 mmol, 1 eq.) and rac-(1R,5S)-8-methyl-3,8-diazabicyclo[3.2.1]octane dihydrochloride (24 mg, 0.12 mmol, 1.2 eq.) as reactants afforded the title compound (15 mg, 27%) as an off-white solid: ¹H NMR (500 MHz, DMSO-d₆) δ 8.19 (s, 1H), 7.90 (dd, J=8.1, 2.1 Hz, 1H), 7.43-7.23 (m, 4H), 6.79-6.73 (m, 1H), 5.10-4.79 (m, 6H), 3.54 (dd, J=10.8, 2.4 Hz, 2H), 3.52 (s, 3H), 3.39-3.23 (m, 3H), 2.95 (dd, J=10.6, 2.3 Hz, 1H), 2.91 (s, 2H), 2.25 (s, 3H), 2.01-1.90 (m, 2H), 1.66 (d, J=7.6 Hz, 2H); LCMS: C₂₉H₃₁F₃N₆O₂ requires: 552, found: m/z=553 [M+H]⁺.

### Example 389: (S)-6-(2,4-Dimethylpiperazin-1-yl)-2-(3-(3-((4-methyl-4H-1,2,4-triazol-3-yl)methyl)oxetan-3-yl)phenyl)-4-(trifluoromethyl)isoindolin-1-one (389)

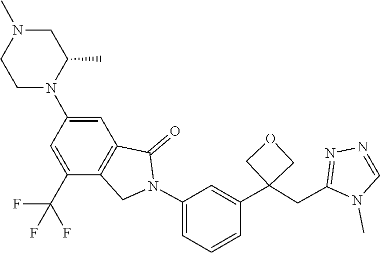 [SMILES: C[C@H]1CN(C)CCN1c1cc2c(c(C(F)(F)F)c1)CN(c1cccc(C3(Cc4nncn4C)COC3)c1)C2=O]

The C—N coupling reaction with potassium phosphate was carried out in a similar fashion as for 386 using 6-bromo-2-(3-(3-((4-methyl-4H-1,2,4-triazol-3-yl)methyl)oxetan-3-yl)phenyl)-4-(trifluoromethyl)isoindolin-1-one (50 mg, 0.10 mmol, 1 eq.) and (3S)-1,3-dimethylpiperazine (11 mg, 0.10 mmol, 1 eq.) as reactants afforded the title compound (11 mg, 20%) as an off-white solid: 1H NMR (500 MHz, DMSO-d₆) δ 8.19 (s, 1H), 7.92-7.86 (m, 1H), 7.46-7.29 (m, 4H), 6.76 (d, J=7.6 Hz, 1H), 5.02-4.83 (m, 6H), 4.26 (s, 1H), 3.52 (s, 3H), 3.49 (s, 1H), 3.11-3.06 (m, 1H), 2.90 (s, 2H), 2.87 (d, J=10.6 Hz, 1H), 2.71 (d, J=11.0 Hz, 1H), 2.29-2.24 (m, 1H), 2.23 (s, 3H), 2.09-2.03 (m, 1H), 1.08 (d, J=6.5 Hz, 3H); LCMS: C₂₈H₃₁F₃N₆O₂ requires: 540, found: m/z=541 [M+H]⁺.

### Example 390: (R)-6-(2,4-Dimethylpiperazin-1-yl)-2-(3-(3-((4-methyl-4H-1,2,4-triazol-3-yl)methyl)oxetan-3-yl)phenyl)-4-(trifluoromethyl)isoindolin-1-one (390)

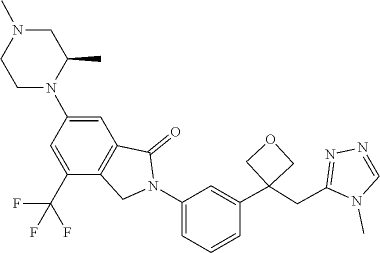 [SMILES: C[C@@H]1CN(C)CCN1c1cc2c(c(C(F)(F)F)c1)CN(c1cccc(C3(Cc4nncn4C)COC3)c1)C2=O]

The C—N coupling reaction with potassium phosphate was carried out in a similar fashion as for 386 using 6-bromo-2-(3-(3-((4-methyl-4H-1,2,4-triazol-3-yl)methyl)oxetan-3-yl)phenyl)-4-(trifluoromethyl)isoindolin-1-one (50 mg, 0.10 mmol, 1 eq.) and OR)-1,3-dimethylpiperazine (11 mg, 0.10 mmol, 1 eq.) as reactants afforded the title compound (11 mg, 20%) as an off-white solid: ¹H NMR (500 MHz, DMSO-d₆) δ 8.19 (s, 1H), 7.89 (dd, J=8.0, 2.1 Hz, 1H), 7.47-7.27 (m, 4H), 6.79-6.73 (m, 1H), 5.10-4.78 (m, 6H), 4.26 (s, 1H), 3.52 (s, 3H), 3.49 (s, 1H), 3.08 (td, J=11.7, 3.2 Hz, 1H), 2.90 (s, 2H), 2.87 (d, J=11.0 Hz, 1H), 2.71 (d, J=10.9 Hz, 1H), 2.29-2.24 (m, 1H), 2.23 (s, 3H), 2.10-2.03 (m, 1H), 1.08 (d, J=6.5 Hz, 3H); LCMS: C₂₈H₃₁F₃N₆O₂ requires: 540, found: m/z=541 [M+H]⁺.

### Example 391: 6-(4-Methyl-2-oxopiperazin-1-yl)-2-(3-(3-((4-methyl-4H-1,2,4-triazol-3-yl)methyl)oxetan-3-yl)phenyl)-4-(trifluoromethyl)isoindolin-1-one (391)

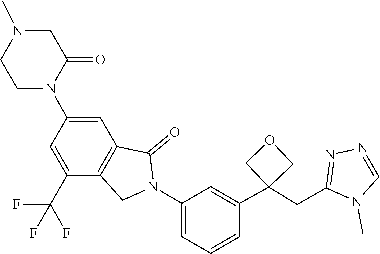 [SMILES: CN1CCN(c2cc3c(c(C(F)(F)F)c2)CN(c2cccc(C4(Cc5nncn5C)COC4)c2)C3=O)C(=O)C1]

The C—N coupling reaction with potassium phosphate was carried out in a similar fashion as for 386 using 6-bromo-2-(3-(3-((4-methyl-4H-1,2,4-triazol-3-yl)methyl)oxetan-3-yl)phenyl)-4-(trifluoromethyl)isoindolin-1-one (50 mg, 0.10 mmol, 1 eq.) and 4-methylpiperazin-2-one (14 mg, 0.12 mmol, 1.2 eq.) as reactants afforded the title compound (19 mg, 36%) as an off-white solid: ¹H NMR (500 MHz, DMSO-d₆) δ 8.20 (s, 1H), 8.07 (dd, J=17.6, 1.8 Hz, 2H), 7.93-7.87 (m, 1H), 7.43-7.33 (m, 2H), 6.79 (dt, J=7.7, 1.2 Hz, 1H), 5.13 (d, J=1.6 Hz, 2H), 4.98 (d, J=6.0 Hz, 2H), 4.90 (d, J=6.1 Hz, 2H), 3.83 (dd, J=6.2, 4.5 Hz, 2H), 3.52 (s, 3H), 3.20 (s, 2H), 2.92 (s, 2H), 2.79 (t, J=5.4 Hz, 2H), 2.32 (s, 3H); LCMS: C₂₇H₂₇F₃N₆O₃ requires: 540, found: m/z=541 [M+H]⁺.

### Example 392 and Example 393: (R)-6-(hydroxymethyl)-2-(3-(1-(4-methyl-4H-1,2,4-triazol-3-yl)propan-2-yl)phenyl)-4-(trifluoromethyl)isoindolin-1-one (392) and (R)-6-((3-hydroxyazetidin-1-yl)methyl)-2-(3-(1-(4-methyl-4H-1,2,4-triazol-3-yl)propan-2-yl)phenyl)-4-(trifluoromethyl)isoindolin-1-one (393)

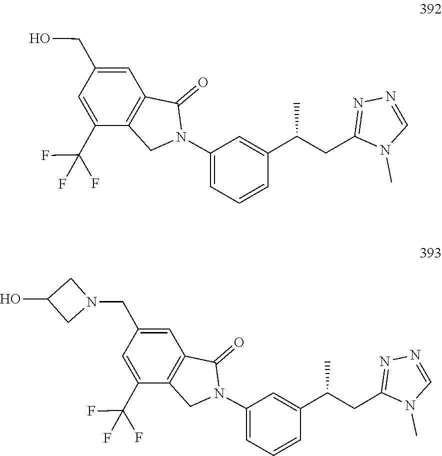 [SMILES: C[C@H](Cc1nncn1C)c1cccc(N2Cc3c(cc(CO)cc3C(F)(F)F)C2=O)c1 ; C[C@H](Cc1nncn1C)c1cccc(N2Cc3c(cc(CN4CC(O)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (48 mg, 0.11 mmol, 1.0 eq.) and azetidin-3-ol (15 mg, 0.21 mmol, 1.8 eq.) as reactants which 392 (18 mg, 37% yield) and 393 (8.0 mg, 15% yield) as colorless solids.

(R)-6-(hydroxymethyl)-2-(3-(1-(4-methyl-4H-1,2,4-triazol-3-yl)propan-2-yl)phenyl)-4-(trifluoromethyl)isoindolin-1-one (392): ¹H NMR (500 MHz, DMSO-d6) δ 8.27 (s, 1H), 8.00 (s, 1H), 7.94 (s, 1H), 7.79 (dd, J=7.8, 1.1 Hz, 2H), 7.35 (td, J=7.6, 0.9 Hz, 1H), 7.10 (dt, J=7.4, 1.3 Hz, 1H), 5.57 (t, J=5.8 Hz, 1H), 5.17 (s, 2H), 4.70 (d, J=5.7 Hz, 2H), 3.45 (s, 3H), 3.00 (dd, J=7.4, 2.3 Hz, 2H), 1.30 (d, J=7.0 Hz, 3H); LCMS: C₂₂H₂₁F₃N₄O₂ requires: 430, found: m/z=431 [M+H]⁺.

(R)-6-((3-hydroxyazetidin-1-yl)methyl)-2-(3-(1-(4-methyl-4H-1,2,4-triazol-3-yl)propan-2-yl)phenyl)-4-(trifluoromethyl)isoindolin-1-one (393): ¹H NMR (500 MHz, DMSO-d6) δ 8.27 (s, 1H), 7.93 (s, 1H), 7.87 (s, 1H), 7.77 (dt, J=6.9, 1.8 Hz, 2H), 7.35 (dd, J=9.0, 7.5 Hz, 1H), 7.10 (dt, J=7.6, 1.3 Hz, 1H), 5.34 (d, J=6.4 Hz, 1H), 5.16 (s, 2H), 4.23 (h, J=6.2 Hz, 1H), 3.76 (s, 2H), 3.55-3.47 (m, 2H), 3.44 (s, 3H), 3.00 (dd, J=7.4, 2.8 Hz, 2H), 2.82 (td, J=6.1, 2.0 Hz, 2H), 1.30 (d, J=6.9 Hz, 3H); LCMS: C₂₅H26F3N₅O₂ requires: 485, found: m/z=486 [M+H]⁺.

### Example 394: 6-(((S)-3-hydroxypyrrolidin-1-yl)methyl)-2-(3-((R)-1-(4-methyl-4H-1,2,4-triazol-3-yl)propan-2-yl)phenyl)-4-(trifluoromethyl)isoindolin-1-one (394)

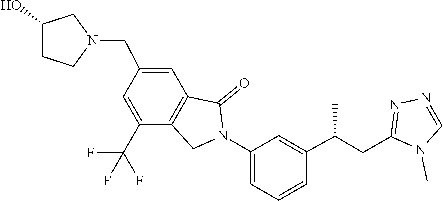 [SMILES: C[C@H](Cc1nncn1C)c1cccc(N2Cc3c(cc(CN4CC[C@H](O)C4)cc3C(F)(F)F)C2=O)c1]

Step 1: Synthesis of (R)-6-bromo-2-(3-(1-(4-methyl-4H-1,2,4-triazol-3-yl)propan-2-yl)phenyl)-4-(trifluoromethyl)isoindolin-1-one: The indolone formation reaction was carried out in a manner similar to Example 260, step 2 using (R)-3-(1-(4-methyl-4H-1,2,4-triazol-3-yl)propan-2-yl)aniline (1.28 g, 5.93 mmol, 1.1 eq.) and methyl 5-bromo-2-(bromomethyl)-3-(trifluoromethyl)benzoate (2.03 g, 5.39 mmol, 1.0 eq.) as reactants to afford the title compound in 1.24 g (48%) yield.

Step 2: Synthesis of (R)-2-(3-(1-(4-methyl-4H-1,2,4-triazol-3-yl)propan-2-yl)phenyl)-4-(trifluoromethyl)-6-vinylisoindolin-1-one: Installation of the vinyl substituent was carried out in a similar fashion as for Example 220, step 1 using (R)-6-bromo-2-(3-(l-(4-methy 1-4H-1,2,4-triazol-3-yl)propan-2-yl)phenyl)-4-(trifluoromethyl)isoindolin-1-one (1.24 g, 2.58 mmol, 1.0 eq.) as the reactant to afford the title compound in 801 mg (73%) yield.

Step 3: Synthesis of (R)-2-(3-(1-(4-methyl-4H-1,2,4-triazol-3-yl)propan-2-yl)phenyl)-3-oxo-7-(trifluoromethyl)isoindoline-5-carbaldehyde: The vinyl group was converted to the aldehyde according to the procedure described for Example 447, step 3 using (R)-2-(3-(1-(4-methyl-4H-1,2,4-triazol-3-yl)propan-2-yl)phenyl)-4-(trifluoromethyl)-6-vinylisoindolin-1-one (448 mg, 1.05 mmol, 1.0 eq.) as the reactant to afford the title compound in 329 mg (67%) yield.

Step 4: Synthesis of 6-(((S)-3-hydroxypyrrolidin-1-yl)methyl)-2-(3-((R)-1-(4-methyl-4H-1,2,4-triazol-3-yl)propan-2-yl)phenyl)-4-(trifluoromethyl)isoindolin-1-one: The reductive animation was carried out in a similar fashion as for Example 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (93 mg, 0.22 mmol, 1.0 eq.) and (S)-pyrrolidin-3-ol (35 μL, 0.42 mmol, 1.9 eq.) as reactants to afford the title compound (55 mg, 50% yield) as a colorless solid: ¹H NMR (500 MHz, DMSO-d6) δ 8.27 (s, 1H), 7.98 (s, 1H), 7.92 (s, 1H), 7.81-7.76 (m, 2H), 7.35 (dd, J=8.8, 7.6 Hz, 1H), 7.13-7.08 (m, 1H), 5.16 (s, 2H), 4.72 (d, J=4.5 Hz, 1H), 4.21 (tq, J=7.4, 3.7 Hz, 1H), 3.77 (q, J=13.7 Hz, 2H), 3.44 (s, 3H), 3.00 (dd, J=7.4, 2.7 Hz, 2H), 2.69 (dd, J=9.7, 6.1 Hz, 1H), 2.63 (q, J=8.1 Hz, 1H), 2.43 (td, J=8.3, 5.7 Hz, 1H), 2.38-2.33 (m, 1H), 2.01 (dq, J=14.2, 7.4 Hz, 1H), 1.56 (qd, J=8.7, 8.2, 4.4 Hz, 1H), 1.30 (d, J=6.9 Hz, 3H); LCMS: C₂₆H₂₈F₃N₅O₂ requires: 499, found: m/z=500 [M+H]⁺.

### Example 395: 6-(((3R,4R)-3-fluoro-4-hydroxypyrrolidin-1-yl)methyl)-2-(3-((R)-1-(4-methyl-4H-1,2,4-triazol-3-yl)propan-2-yl)phenyl)-4-(trifluoromethyl)isoindolin-1-one (395)

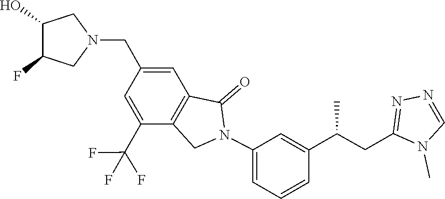 [SMILES: C[C@H](Cc1nncn1C)c1cccc(N2Cc3c(cc(CN4C[C@@H](O)[C@H](F)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (21 mg, 0.048 mmol, 1.0 eq.) and (3R,4R)-4-fluoropyrrolidin-3-ol hydrochloride (43 mg, 0.31 mmol, 6.3 eq.) as reactants to afford the title compound (11 mg, 45% yield) as a colorless solid: ¹H NMR (500 MHz, DMSO-d6) δ 8.27 (s, 1H), 7.98 (s, 1H), 7.92 (s, 1H), 7.82-7.74 (m, 2H), 7.35 (dd, J=8.8, 7.6 Hz, 1H), 7.14-7.08 (m, 1H), 5.35 (d, J=4.7 Hz, 1H), 5.17 (s, 2H), 4.83 (dd, J=53.1, 5.0 Hz, 1H), 4.25-4.11 (m, 1H), 3.91-3.75 (m, 2H), 3.44 (s, 3H), 3.07 (dd, J=9.6, 6.4 Hz, 1H), 3.00 (dd, J=7.4, 2.7 Hz, 2H), 2.89-2.68 (m, 2H), 2.23 (dd, J=9.7, 5.4 Hz, 1H), 1.30 (d, J=6.9 Hz, 3H); LCMS: C₂₆H₂₇F₄N₅O₂ requires: 517, found: m/z=518 [M+H]⁺.

### Example 396: (R)-6-((3,3-difluoropyrrolidin-1-yl)methyl)-2-(3-(1-(4-methyl-4H-1,2,4-triazol-3-yl)propan-2-yl)phenyl)-4-(trifluoromethyl)isoindolin-1-one (396)

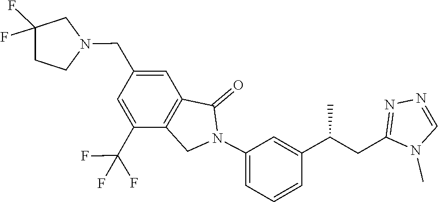 [SMILES: C[C@H](Cc1nncn1C)c1cccc(N2Cc3c(cc(CN4CCC(F)(F)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (26 mg, 0.060 mmol, 1.0 eq.) and 3,3-difluoropyrrolidine hydrochloride (95 mg, 0.66 mmol, 11 eq.) as reactants to afford the title compound (14 mg, 45% yield) as a colorless solid: 1H NMR (500 MHz, DMSO-d₆) δ 8.21 (s, 1H), 7.94 (s, 1H), 7.88 (s, 1H), 7.75-7.70 (m, 2H), 7.30 (dd, J=8.8, 7.7 Hz, 1H), 7.08-7.02 (m, 1H), 5.12 (s, 2H), 3.79 (s, 2H), 3.38 (s, 3H), 2.94 (dd, J=7.4, 2.8 Hz, 2H), 2.85 (t, J=13.3 Hz, 2H), 2.69 (t, J=7.0 Hz, 2H), 2.22 (tt, J=15.0, 7.0 Hz, 2H), 1.24 (d, J=6.9 Hz, 3H); LCMS: C₂₆H₂₆F₅N₅O requires: 519, found: m/z=520 [M+H]⁺.

### Example 397: (R)-6-((3-hydroxy-3-methylazetidin-1-yl(methyl)-2-(3-(1-(4-methyl-4H-1,2,4-triazol-3-yl)propan-2-yl)phenyl)-4-(trifluoromethyl)isoindolin-1-one (397)

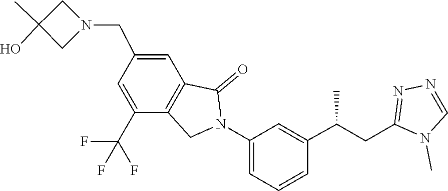 [SMILES: C[C@H](Cc1nncn1C)c1cccc(N2Cc3c(cc(CN4CC(C)(O)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (50 mg, 0.12 mmol, 1.0 eq.) and 3-methylazetidin-3-ol hydrochloride (50 mg, 0.41 mmol, 3.5 eq.) as reactants to afford the title compound (29 mg, 50% yield) as a colorless solid: ¹H NMR (500 MHz, DMSO-d6) δ 8.27 (s, 1H), 7.94 (s, 1H), 7.88 (s, 1H), 7.83-7.74 (m, 2H), 7.35 (t, J=7.8 Hz, 1H), 7.10 (d, J=7.8 Hz, 1H), 5.20 (s, 1H), 5.16 (s, 2H), 3.79 (s, 2H), 3.44 (s, 3H), 3.20 (s, 2H), 3.01 (dd, J=7.4, 2.8 Hz, 2H), 2.98-2.92 (m, 2H), 3.05-2.86 (m, 2H), 1.38 (s, 3H), 1.30 (d, J=6.9 Hz, 3H); LCMS: C₂₆H₂₈F₃N₅O₂ requires: 499, found: m/z=500 [M+H]⁺.

### Example 398: 6-(((S)-3-hydroxy-3-methylpyrrolidin-1-yl)methyl)-2-(3-((R)-1-(4-methyl-4H-1,2,4-triazol-3-yl)propan-2-yl)phenyl)-4-(trifluoromethyl)isoindolin-1-one (398)

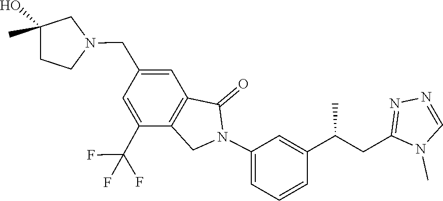 [SMILES: C[C@H](Cc1nncn1C)c1cccc(N2Cc3c(cc(CN4CC[C@](C)(O)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (50 mg, 0.12 mmol, 1.0 eq.) and (S)-3-methylpyrrolidin-3-ol hydrochloride (51 mg, 0.37 mmol, 3.2 eq.) as reactants to afford the title compound (34 mg, 57% yield) as a colorless solid: ¹H NMR (500 MHz, DMSO-d6) δ 8.27 (s, 1H), 7.98 (s, 1H), 7.92 (s, 1H), 7.82-7.75 (m, 2H), 7.35 (dd, J=8.8, 7.6 Hz, 1H), 7.10 (dd, J=7.5, 1.4 Hz, 1H), 5.16 (s, 2H), 4.58 (s, 1H), 3.84-3.71 (m, 2H), 3.44 (s, 3H), 3.08-2.94 (m, 2H), 2.73-2.64 (m, 1H), 2.43 (d, J=9.2 Hz, 1H), 1.82-1.66 (m, 2H), 1.30 (d, J=6.9 Hz, 3H), 1.24 (s, 3H); LCMS: C₂₇H₃₀F₃N₅O₂ requires: 513, found: m/z=514 [M+H]⁺. LCMS: C₂₇H₃₀F₃N₅O₂ requires: 513, found: m/z=514 [M+H]⁺.

### Example 399: 6-(((S)-3-fluoropyrrolidin-1-yl)methyl)-2-(3-((R)-1-(4-methyl-4H-1,2,4-triazol-3-yl)propan-2-yl)phenyl)-4-(trifluoromethyl)isoindolin-1-one (399)

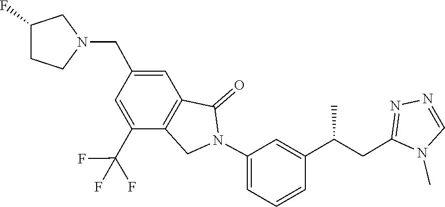 [SMILES: C[C@H](Cc1nncn1C)c1cccc(N2Cc3c(cc(CN4CC[C@H](F)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (49 mg, 0.11 mmol, 1.0 eq.) and (S)-3-fluoropyrrolidine hydrochloride (44 mg, 0.35 mmol, 3.1 eq.) as reactants to afford the title compound (34 mg, 60% yield) as a colorless solid: ¹H NMR (500 MHz, DMSO-d6) δ 8.27 (s, 1H), 8.00 (s, 1H), 7.94 (s, 1H), 7.82-7.76 (m, 2H), 7.38-7.32 (m, 1H), 7.11 (dt, J=7.7, 1.3 Hz, 1H), 5.28-5.15 (m, 1H), 5.17 (s, 2H), 3.83 (s, 2H), 3.44 (s, 3H), 3.05-2.95 (m, 2H), 2.87-2.75 (m, 2H), 2.72-2.58 (m, 1H), 2.42-2.34 (m, 1H), 2.16 (ddq, J=27.9, 14.2, 6.7 Hz, 1H), 1.90 (ddt, J=29.6, 14.3, 7.0 Hz, 1H), 1.30 (d, J=6.9 Hz, 3H); LCMS: C₂₆H₂₇F₄N₅O requires: 501, found: m/z=502 [M+H]⁺.

### Example 400: 6-(((R)-3-fluoropyrrolidin-1-yl)methyl)-2-(3-((R)-1-(4-methyl-4H-1,2,4-triazol-3-yl)propan-2-yl)phenyl)-4-(trifluoromethyl)isoindolin-1-one (400)

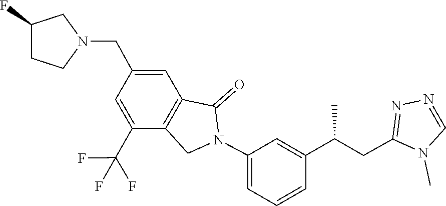 [SMILES: C[C@H](Cc1nncn1C)c1cccc(N2Cc3c(cc(CN4CC[C@@H](F)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (49 mg, 0.11 mmol, 1.0 eq.) and (R)-3-fluoropyrrolidine hydrochloride (44 mg, 0.35 mmol, 3.1 eq.) as reactants to afford the title compound (37 mg, 65% yield) as a colorless solid: ¹H NMR (500 MHz, DMSO-d6) δ 8.28 (s, 1H), 8.01 (d, J=1.3 Hz, 1H), 7.95 (s, 1H), 7.80 (dp, J=5.4, 1.9 Hz, 2H), 7.40-7.34 (m, 1H), 7.12 (d, J=7.7 Hz, 1H), 5.30-5.16 (m, 1H), 5.19 (s, 2H), 3.85 (s, 2H), 3.46 (s, 3H), 3.10-2.95 (m, 2H), 2.92-2.76 (m, 2H), 2.76-2.61 (m, 1H), 2.43-2.34 (m, 1H), 2.18 (ddq, J=27.9, 14.4, 6.9 Hz, 1H), 1.92 (ddt, J=29.7, 14.3, 7.0 Hz, 1H), 1.32 (d, J=6.9 Hz, 3H); LCMS: C₂₆H₂₇F₄N₅O requires: 501, found: m/z=502 [M+H]⁺.

### Example 401: (R)-6-((3-fluoroazetidin-1-yl)methyl)-2-(3-(1-(4-methyl-4H-1,2,4-triazol-3-yl)propan-2-yl)phenyl)-4-(trifluoromethyl)isoindolin-1-one (401)

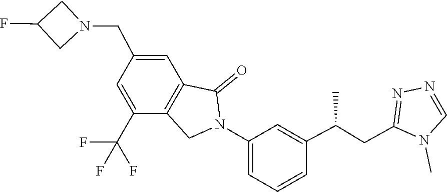 [SMILES: C[C@H](Cc1nncn1C)c1cccc(N2Cc3c(cc(CN4CC(F)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (49 mg, 0.11 mmol, 1.0 eq.) and 3-fluoroazetidine hydrochloride (40 mg, 0.36 mmol, 3.1 eq.) as reactants to afford the title compound (16 mg, 29% yield) as a colorless solid: ¹H NMR (500 MHz, DMSO-d6) δ 8.27 (s, 1H), 7.96 (s, 1H), 7.90 (s, 1H), 7.81-7.76 (m, 2H), 7.39-7.31 (m, 1H), 7.10 (d, J=7.8 Hz, 1H), 5.28-5.13 (m, 1H), 5.15 (d, J=6.7 Hz, 2H), 3.84 (s, 2H), 3.67-3.53 (m, 2H), 3.44 (s, 3H), 3.26-3.21 (m, 1H), 3.21-3.15 (m, 1H), 3.06-2.94 (m, 2H), 1.30 (d, J=6.9 Hz, 3H); LCMS: C₂₅H₂₅F₄N₅O requires: 487, found: m/z=488 [M+H]⁺.

### Example 402: (R)-6-((3,3-difluoroazetidin-1-yl)methyl)-2-(3-(1-(4-methyl-4H-1,2,4-triazol-3-yl)propan-2-yl)phenyl)-4-(trifluoromethyl)isoindolin-1-one (402)

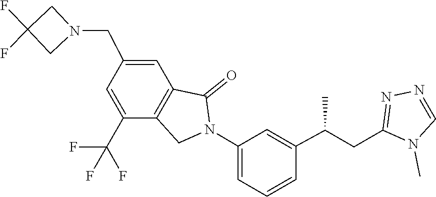 [SMILES: C[C@H](Cc1nncn1C)c1cccc(N2Cc3c(cc(CN4CC(F)(F)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (13 mg, 0.030 mmol, 1.0 eq.) and 3,3-difluoroazetidine hydrochloride (12 mg, 0.090 mmol, 3.0 eq.) as reactants to afford the title compound (7.0 mg, 46% yield) as a colorless solid: ¹H NMR (500 MHz, DMSO-d6) δ 8.27 (s, 1H), 8.00 (s, 1H), 7.93 (s, 1H), 7.81-7.76 (m, 2H), 7.39-7.32 (m, 1H), 7.11 (d, J=7.6 Hz, 1H), 5.17 (s, 2H), 3.94 (s, 2H), 3.67 (t, J=12.5 Hz, 4H), 3.44 (s, 3H), 3.07-2.92 (m, 2H), 1.30 (d, J=6.9 Hz, 3H); LCMS: C₂₅H₂₄F₅N₅O requires: 505, found: m/z=506 [M+H]⁺.

### Example 403 and Example 404: 6-((R)-1-((S)-3-fluoropyrrolidin-1-yl)ethyl)-2-(3-((R)-1-(4-methyl-4H-1,2,4-triazol-3-yl)propan-2-yl)phenyl)-4-(trifluoromethyl)isoindolin-1-one (403) and 6-((S)-1-((S)-3-fluoropyrrolidin-1-yl)ethyl)-2-(3-((R)-1-(4-methyl-4H-1,2,4-triazol-3-yl)propan-2-yl)phenyl)-4-(trifluoromethyl)isoindolin-1-one (404)

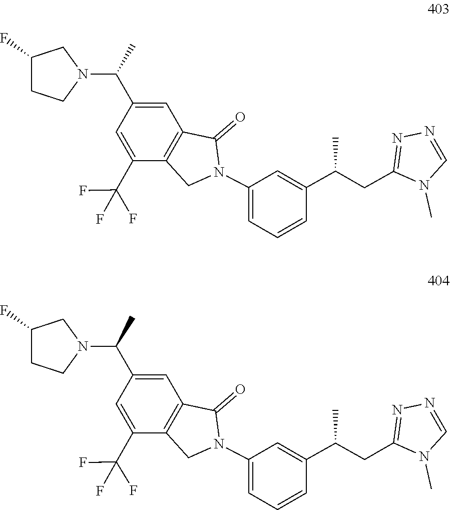 [SMILES: C[C@H](Cc1nncn1C)c1cccc(N2Cc3c(cc([C@@H](C)N4CC[C@H](F)C4)cc3C(F)(F)F)C2=O)c1 ; C[C@H](Cc1nncn1C)c1cccc(N2Cc3c(cc([C@H](C)N4CC[C@H](F)C4)cc3C(F)(F)F)C2=O)c1]

Step 1: Synthesis of (R)-6-acetyl-2-(3-(1-(4-methyl-4H-1,2,4-triazol-3-yl)propan-2-yl)phenyl)-4-(trifluoromethyl)isoindolin-1-one: The methyl ketone was synthesized according to the procedure for 459, step 1 using (R)-6-bromo-2-(3-(1-(4-methyl-4H-1,2,4-triazol-3-yl)propan-2-yl)phenyl)-4-(trifluoromethyl)isoindolin-1-one (821 mg, 1.21 mmol, 1.0 eq.) as the reactant to afford the title compound in 216 mg (40%) yield.

Step 2: Synthesis of 6-(1-((S)-3-fluoropyrrolidin-1-yl)ethyl)-2-(3-((R)-1-(4-methyl-4H-1,2,4-triazol-3-yl)propan-2-yl)phenyl)-4-(trifluoromethyl)isoindolin-1-one: Reductive amination of the methyl ketone was carried out according to the procedure of the procedure for 459, step 2 using (R)-6-acetyl-2-(3-(1-(4-methyl-4H-1,2,4-triazol-3-yl)propan-2-yl)phenyl)-4-(trifluoromethyl)isoindolin-1-one (40 mg, 0.090 mmol, 1.0 eq.) and (S)-3-fluoropyrrolidine hydrochloride (61 mg, 0.49 mmol, 5.4 eq.) as reactants to afford the title compound in 18 mg (38%) yield.

Step 3: 6-((R)-1-((S)-3-fluoropyrrolidin-1-yl)ethyl)-2-(3-((R)-1-(4-methyl-4H-1,2,4-triazol-3-yl)propan-2-yl)phenyl)-4-(trifluoromethyl)isoindolin-1-one and 6-((S)-1-((S)-3-fluoropyrrolidin-1-yl)ethyl)-2-(3-((R)-1-(4-methyl-4H-1,2,4-triazol-3-yl)propan-2-yl)phenyl)-4-(trifluoromethyl)isoindolin-1-one: The diastereomers of 6-(1-((S)-3-fluoropyrrolidin-1-yl)ethyl)-2-(3-((R)-1-(4-methyl-4H-1,2,4-triazol-3-yl)propan-2-yl)phenyl)-4-(trifluoromethyl)isoindolin-1-one (50 mg) was separated using column IG with CO₂ and methanol as mobile phase to afford:

6-((R)-1-((S)-3-fluoropyrrolidin-1-yl)ethyl)-2-(3-((R)-1-(4-methyl-4H-1,2,4-triazol-3-yl)propan-2-yl)phenyl)-4-(trifluoromethyl)isoindolin-1-one: (16 mg)¹H NMR (500 MHz, Acetone-d6) δ 8.09 (s, 1H), 8.06 (s, 1H), 7.98 (s, 1H), 7.94-7.86 (m, 2H), 7.36 (t, J=7.7 Hz, 1H), 7.11 (d, J=7.6 Hz, 1H), 5.28-5.10 (m, 1H), 5.16 (s, 2H), 3.64 (d, J=7.0 Hz, 1H), 3.50 (s, 3H), 3.42 (d, J=7.1 Hz, 1H), 3.06 (t, J=7.1 Hz, 2H), 2.93 (dd, J=27.3, 11.5 Hz, 1H), 2.73 (dd, J=37.0, 6.7 Hz, 2H), 2.53 (d, J=6.3 Hz, 1H), 2.23-2.11 (m, 1H), 2.01-1.90 (m 1H), 1.44 (d, J=6.6 Hz, 3H), 1.40 (d, J=7.0 Hz, 3H); LCMS: C₂₇H29F₄N₅O requires: 515, found: m/z=516 [M+H]⁺.

6-((S)-1-((S)-3-fluoropyrrolidin-1-yl)ethyl)-2-(3-((R)-1-(4-methyl-4H-1,2,4-triazol-3-yl)propan-2-yl)phenyl)-4-(trifluoromethyl)isoindolin-1-one: (11 mg)¹H NMR (500 MHz, Acetone-d6) δ 8.09 (s, 1H), 8.06 (s, 1H), 7.98 (s, 1H), 7.91-7.89 (m, 2H), 7.36 (dd, J=8.8, 7.6 Hz, 1H), 7.11 (d, J=7.7 Hz, 1H), 5.26-5.07 (m, 3H), 3.61 (d, J=6.6 Hz, 1H), 3.49 (s, 3H), 3.42 (d, J=7.1 Hz, 1H), 3.06 (t, J=7.1 Hz, 2H), 2.97 (d, J=6.3 Hz, 1H), 2.78-2.65 (m, 2H), 2.36 (d, J=8.2 Hz, 1H), 2.20 (dd, J=20.3, 6.2 Hz, 1H), 1.97 (dd, J=31.2, 7.2 Hz, 1H), 1.44 (d, J=6.6 Hz, 3H), 1.40 (d, J=6.9 Hz, 3H); LCMS: C₂₇H29F₄N₅O requires: 515, found: m/z=516 [M+H]⁺.

### Example 405 and Example 406: 6-((R)-1-(3-hydroxyazetidin-1-yl)ethyl)-2-(3-((R)-1-(4-methyl-4H-1,2,4-triazol-3-yl)propan-2-yl)phenyl)-4-(trifluoromethyl)isoindolin-1-one (405) and 6-((S)-1-(3-hydroxyazetidin-1-yl)ethyl)-2-(3-((R)-1-(4-methyl-4H-1,2,4-triazol-3-yl)propan-2-yl)phenyl)-4-(trifluoromethyl)isoindolin-1-one (406)

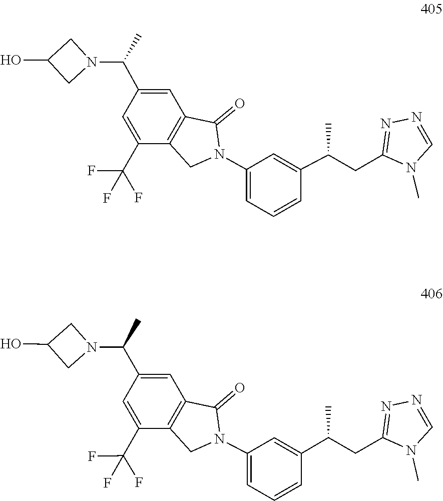 [SMILES: C[C@H](Cc1nncn1C)c1cccc(N2Cc3c(cc([C@@H](C)N4CC(O)C4)cc3C(F)(F)F)C2=O)c1 ; C[C@H](Cc1nncn1C)c1cccc(N2Cc3c(cc([C@H](C)N4CC(O)C4)cc3C(F)(F)F)C2=O)c1]

Step 1: Synthesis of 6-(1-(3-hydroxyazetidin-1-yl)ethyl)-2-(3-((R)-1-(4-methyl-4H-1,2,4-triazol-3-yl)propan-2-yl)phenyl)-4-(trifluoromethyl)isoindolin-1-one: Reductive amination of the methyl ketone was carried out according to the procedure for 459, step 2 using (R)-6-acetyl-2-(3-(1-(4-methyl-4H-1,2,4-triazol-3-yl)propan-2-yl)phenyl)-4-(trifluoromethyl)isoindolin-1-one (24 mg, 0.054 mmol, 1.0 eq.) and azetidin-3-ol (42 mg, 0.58 mmol, 11 eq.) as reactants to afford the title compound in 18 mg (68%) yield.

Step 2: Synthesis of 6-((R)-1-(3-hydroxyazetidin-1-yl)ethyl)-2-(3-((R)-1-(4-methyl-4H-1,2,4-triazol-3-yl)propan-2-yl)phenyl)-4-(trifluoromethyl)isoindolin-1-one and 6-((S)-1-(3-hydroxyazetidin-1-yl)ethyl)-2-(3-((R)-1-(4-methyl-4H-1,2,4-triazol-3-yl)propan-2-yl)phenyl)-4-(trifluoromethyl)isoindolin-1-one: The diastereomers of 6-(1-(3-hydroxyazetidin-1-yl)ethyl)-2-(3-((R)-1-(4-methyl-4H-1,2,4-triazol-3-yl)propan-2-yl)phenyl)-4-(trifluoromethyl)isoindolin-1-one (18 mg) was separated using column IA with CO₂ and methanol/acetonitrile (7:3) as mobile phase to afford:

6-((R)-1-(3-hydroxyazetidin-1-yl)ethyl)-2-(3-((R)-1-(4-methyl-4H-1,2,4-triazol-3-yl)propan-2-yl)phenyl)-4-(trifluoromethyl)isoindolin-1-one: (4.6 mg)¹H NMR (500 MHz, DMSO-d6) δ 8.26 (s, 1H), 7.97 (s, 1H), 7.90 (s, 1H), 7.77 (dt, J=7.3, 1.6 Hz, 2H), 7.38-7.31 (m, 1H), 7.13-7.08 (m, 1H), 5.29 (s, 2H), 5.15 (s, 2H), 4.18 (p, J=6.1 Hz, 1H), 3.61 (t, J=6.3 Hz, 1H), 3.54 (q, J=6.4 Hz, 1H), 3.44 (s, 3H), 3.28-3.14 (m, 1H), 3.04-2.92 (m, 2H), 2.82 (t, J=6.5 Hz, 1H), 2.69-2.58 (m, 1H), 1.30 (d, J=6.9 Hz, 3H), 1.16 (d, J=6.4 Hz, 3H); LCMS: C₂₆H₂₈F₃N₅O₂ requires: 499, found: m/z=500 [M+H]⁺.

6-((S)-1-(3-hydroxyazetidin-1-yl)ethyl)-2-(3-((R)-1-(4-methyl-4H-1,2,4-triazol-3-yl)propan-2-yl)phenyl)-4-(trifluoromethyl)isoindolin-1-one: (5.1 mg)¹H NMR (500 MHz, DMSO-d6) δ 8.26 (s, 1H), 7.97 (s, 1H), 7.90 (s, 1H), 7.77 (dt, J=6.9, 1.7 Hz, 2H), 7.35 (dd, J=8.9, 7.5 Hz, 1H), 7.10 (d, J=7.7 Hz, 1H), 5.15 (s, 2H), 4.18 (p, J=6.2 Hz, 1H), 3.61 (t, J=6.3 Hz, 1H), 3.54 (q, J=6.4 Hz, 1H), 3.44 (s, 3H), 3.16 (s, 1H), 3.06-2.94 (m, 2H), 2.82 (t, J=6.5 Hz, 1H), 2.68-2.58 (m, 1H), 1.30 (d, J=6.9 Hz, 3H), 1.16 (d, J=6.4 Hz, 3H); LCMS: C₂₆H₂₈F₃N₅O₂ requires: 499, found: m/z=500 [M+H]⁺.

### Example 407: 6-((S)-3-Hydroxypyrrolidine-1-carbonyl)-2-(3-((R)-1-(4-methylisoxazol-3-yl)propan-2-yl)phenyl)-4-(trifluoromethyl)isoindolin-1-one (407)

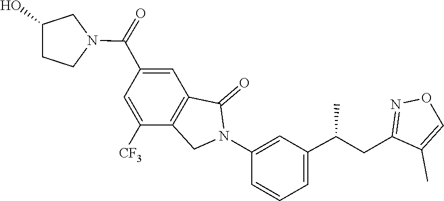 [SMILES: Cc1conc1C[C@@H](C)c1cccc(N2Cc3c(cc(C(=O)N4CC[C@H](O)C4)cc3C(F)(F)F)C2=O)c1]

Step 1: Synthesis of (R)-2-(3-(1-(4-methylisoxazol-3-yl)propan-2-yl)phenyl)-3-oxo-7-(trifluoromethyl)isoindoline-5-carboxylic acid. The indolone formation reaction was carried out in a manner similar to 260, step 2 using 4-(bromomethyl)-3-(methoxycarbonyl)-5-(trifluoromethyl)benzoic acid (76 mg, 0.22 mmol, 1 eq.) and (R)-3-(1-(4-methylisoxazol-3-yl)propan-2-yl)aniline (48 mg, 0.22 mmol, 1 eq.) as reactants afforded the title compound (trifluoroacetic acid salt, 29 mg, 30%) as an off-white solid.

Step 2: Synthesis of 6-((S)-3-Hydroxypyrrolidine-1-carbonyl)-2-(3-((R)-1-(4-methylisoxazol-3-yl)propan-2-yl)phenyl)-4-(trifluoromethyl)isoindolin-1-one. The amide bond formation reaction was carried out in a similar fashion as for 184 using (R)-2-(3-(1-(4-methylisoxazol-3-yl)propan-2-yl)phenyl)-3-oxo-7-(trifluoromethyl)isoindoline-5-carboxylic acid (29 mg, 0.066 mmol, 1 eq.) and (3S)-pyrrolidin-3-ol (17 mg, 0.20 mmol, 3 eq.) as reactants afforded the title compound (24 mg, 70%) as an off-white solid: ¹H NMR (500 MHz, DMSO-d₆) δ 8.50 (d, J=1.3 Hz, 1H), 8.15-8.09 (m, 2H), 7.86 (t, J=2.0 Hz, 1H), 7.78 (dd, J=8.1, 2.2 Hz, 1H), 7.38 (t, J=7.9 Hz, 1H), 7.14 (dt, J=7.7, 1.2 Hz, 1H), 5.27 (s, 2H), 5.10-4.92 (m, 1H), 4.38-4.22 (s, 1H), 3.69-3.54 (m, 2H), 3.44 (d, J=12.3 Hz, 1H), 3.21 (h, J=8.0, 7.6 Hz, 1H), 2.92 (d, J=7.5 Hz, 3H), 2.05-1.72 (m, 4H), 1.30 (d, J=6.9 Hz, 3H); LCMS: C₂₇H₂₆F₃N₃O₄ requires: 513, found: m/z=514 [M+H]⁺.

### Example 408: 2-(3-((1S,2R)-2-(4-methyl-4H-1,2,4-triazol-3-yl)cyclopropyl)phenyl)-3-oxo-7-(trifluoromethyl)isoindoline-5-carbonitrile (408)

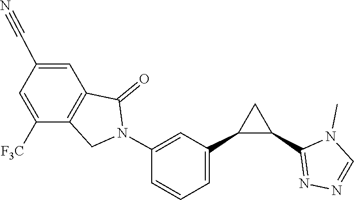 [SMILES: Cn1cnnc1[C@@H]1C[C@@H]1c1cccc(N2Cc3c(cc(C#N)cc3C(F)(F)F)C2=O)c1]

3-(1S,2R)-2-(4-methyl-4H-1,2,4-triazol-3-yl)cyclopropyl aniline (50 mg, 0.23 mmol, 1 eq.) and methyl 2-(bromomethyl)-5-cyano-3-(trifluoromethyl)benzoate (75 mg, 0.23 mmol, 1 eq.) were coupled (see Example 260, Step 2) to afford 58 mg of the title compound as an off-white solid: ¹H NMR (500 MHz, DMSO-d₆) δ 8.81-8.69 (m, 1H), 8.63 (s, 1H), 8.56 (s, 1H), 7.70 (dd, J=8.2, 2.2 Hz, 1H), 7.66 (t, J=2.0 Hz, 1H), 7.24 (td, J=7.9, 2.0 Hz, 1H), 6.91-6.83 (m, 1H), 5.27-5.12 (m, 2H), 3.62 (s, 3H), 2.94-2.81 (m, 1H), 2.77-2.64 (m, 1H), 2.07-1.95 (m, 1H), 1.78-1.65 (m, 1H); LCMS: C₂₂H₁₆F₃N₅O₂ requires: 423, found: m/z=424 [M+H]⁺.

### Example 409: 6-(((S)-3-fluoropyrrolidin-1-yl)methyl)-2-(3-((1S,2R)-2-(4-methyl-4H-1,2,4-triazol-3-yl)cyclopropyl)phenyl)-4-(trifluoromethyl)isoindolin-1-one (409)

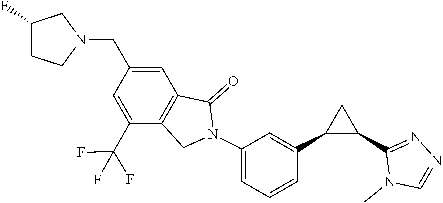 [SMILES: Cn1cnnc1[C@@H]1C[C@@H]1c1cccc(N2Cc3c(cc(CN4CC[C@H](F)C4)cc3C(F)(F)F)C2=O)c1]

Step 1: Synthesis of 6-bromo-2-(3-((1S,2R)-2-(4-methyl-4H-1,2,4-triazol-3-yl)cyclopropyl)phenyl)-4-(trifluoromethyl)isoindolin-1-one: The indolone formation reaction was carried out in a manner similar to 260, step 2 using 3-((1S,2R)-2-(4-methyl-4H-1,2,4-triazol-3-yl)cyclopropyl)aniline (767 mg, 3.58 mmol, 1.0 eq.) and methyl 5-bromo-2-(bromomethyl)-3-(trifluoromethyl)benzoate (1.37 g, 3.65 mmol, 1.0 eq.) as reactants to afford the title compound (1.26 g, 74%).

Step 2: Synthesis of 2-(3-((1S,2R)-2-(4-methyl-4H-1,2,4-triazol-3-yl)cyclopropyl)phenyl)-4-(trifluoromethyl)-6-vinylisoindolin-1-one: Installation of the vinyl substituent was carried out in a similar fashion as for 220, step 1 using 6-bromo-2-(3-((1S,2R)-2-(4-methyl-4H-1,2,4-triazol-3-yl)cyclopropyl)phenyl)-4-(trifluoromethyl)isoindolin-1-one (752 mg, 1.58 mmol, 1.0 eq.) as the reagent to afford the title compound (543 mg, 81%).

Step 3: Synthesis of 2-(3-((1S,2R)-2-(4-methyl-4H-1,2,4-triazol-3-yl)cyclopropyl)phenyl)-3-oxo-7-(trifluoromethyl)isoindoline-5-carbaldehyde: The vinyl group was converted to the aldehyde according to the procedure described for 447, step 3 using 2-(3-((1S,2R)-2-(4-methyl-4H-1,2,4-triazol-3-yl (cyclopropyl)phenyl)-4-(trifluoromethyl)-6-vinylisoindolin-1-one (543 mg, 1.28 mmol, 1.0 eq.) as the reactant to afford the title compound (365 mg, 67%).

Step 4: Synthesis of 6-(((S)-3-fluoropyrrolidin-1-yl)methyl)-2-(3-((1S,2R)-2-(4-methyl-4H-1,2,4-triazol-3-yl)cyclopropyl)phenyl)-4-(trifluoromethyl)isoindolin-1-one: The reductive animation was carried out in a similar fashion as for 447, step 4 using 2-(3-((1S,2R)-2-(4-methyl-4H-1,2,4-triazol-3-yl)cyclopropyl)phenyl)-3-oxo-7-(trifluoromethyl)isoindoline-5-carbaldehyde (41 mg, 0.095 mmol, 1.0 eq.) and (S)-3-fluoropyrrolidine hydrochloride (37 mg, 0.29 mmol, 3.1 eq.) as reactants to afford the title compound (28 mg, 59% yield) as a colorless solid: ¹H NMR (500 MHz, DMSO-d6) δ 8.13 (s, 1H), 7.98 (s, 1H), 7.93 (s, 1H), 7.70 (ddd, J=8.1, 2.4, 1.0 Hz, 1H), 7.54 (t, J=1.9 Hz, 1H), 7.14 (t, J=8.0 Hz, 1H), 6.73 (d, J=7.7 Hz, 1H), 5.21 (dt, J=55.6, 6.0 Hz, 1H), 5.11-4.93 (m, 2H), 3.82 (s, 2H), 3.41 (s, 3H), 2.89-2.73 (m, 2H), 2.73-2.59 (m, 2H), 2.41-2.32 (m, 1H), 2.17 (ddt, J=27.8, 13.7, 7.0 Hz, 1H), 1.99-1.77 (m, 2H), 1.58 (td, J=8.5, 5.0 Hz, 1H); LCMS: C₂₆H₂₅F₄N₅O requires: 499, found: m/z=500 [M+H]⁺.

### Example 410: 6-{[(3S)-3-fluoropyrrolidin-1-yl]methyl}-2-(3-{3-[(4-methyl-4H-1,2,4-triazol-3-yl)methyl]oxetan-3-yl}phenyl)-4-(trifluoromethyl)-2,3-dihydro-1H-isoindol-1-one (410)

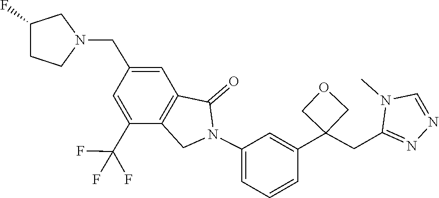 [SMILES: Cn1cnnc1CC1(c2cccc(N3Cc4c(cc(CN5CC[C@H](F)C5)cc4C(F)(F)F)C3=O)c2)COC1]

The reductive amination was carried out in a similar fashion as for 447, step 4 using 2-(3-{3-[(4-methyl-4H-1,2,4-triazol-3-yl)methyl]oxetan-3-yl}phenyl)-3-oxo-7-(trifluoromethyl)-2,3-dihydro-1H-isoindole-5-carbaldehyde (40 mg, 0.09 mmol, 1.0 eq.) and (3R)-pyrrolidine-3-carbonitrile hydrochloride (50 mg, 0.23 mmol, 1.0 eq.) as reactants afford the title compound (12 mg, 26%) as an off-white solid: 1H NMR (500 MHz, Chloroform-d) δ 8.07 (s, 1H), 7.90 (d, J=13.0 Hz, 2H), 7.57-7.48 (m, 2H), 7.35 (t, J=7.9 Hz, 1H), 6.64 (dd, J=7.6, 1.6 Hz, 1H), 5.28 (s, 1H), 5.17 (d, J=6.1 Hz, 2H), 5.10 (d, J=6.1 Hz, 2H), 4.95 (s, 2H), 3.86 (s, 2H), 3.62 (s, 2H), 2.89 (s, 3H), 2.54 (s, 2H), 2.30-1.94 (m, 4H); LCMS: C₂₇H₂₇F₄N₅O₂ requires: 529, found: m/z=530 [M+H]⁺.

### Example 411: 6-((3,3-difluoropyrrolidin-1-yl)methyl)-2-(3-(3-((4-methyl-4H-1,2,4-triazol-3-yl)methyl)oxetan-3-yl)phenyl)-4-(trifluoromethyl)isoindolin-1-one (411)

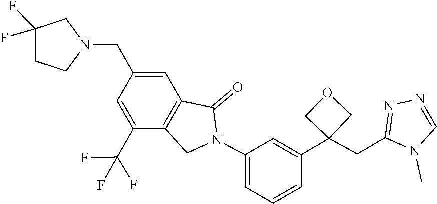 [SMILES: Cn1cnnc1CC1(c2cccc(N3Cc4c(cc(CN5CCC(F)(F)C5)cc4C(F)(F)F)C3=O)c2)COC1]

Step 1: Synthesis of 6-((3,3-difluoropyrrolidin-1-yl)methyl)-2-(3-(3-((4-methyl-4H-1,2,4-triazol-3-yl)methyl)oxetan-3-yl)phenyl)-4-(trifluoromethyl)isoindolin-1-one: 3,3-Difluoropyrrolidine hydrochloride (78 mg, 0.54 mmol, 4.9 eq.) and triethylamine (70 μL, 0.50 mmol, 4.5 eq.) was suspended to dichloromethane (0.5 mL) and the mixture was stirred and sonicated to obtain a uniform suspension. This mixture was added to Example Z (51 mg, 0.11 mmol, 1.0 eq.) at rt using dichloromethane (0.5 mL). The mixture was stirred for 1 h before sodium triacetoxyborohydride (58 mg, 0.27 mmol) was added. The reaction was diluted with methanol (0.5 mL) and sodium cyanoborohydride (14 mg, 0.22 mmol, 2.0 eq.) was added. After stirring for 1 hour, the reaction was quenched with a few drops of 1N-hydrochloric acid. After the bubbling had ceased, the reaction was basified with sat. sodium bicarbonate and the product was extracted using chloroform: isopropyl alcohol (2:1) mixture (×3). The combined organic layers were dried, filtered and concentrated. The residue was purified by reverse phase HPLC using a gradient of acetonitrile in water with 0.1% trifluoroacetic acid to afford the title compound as a colorless solid 32 mg (52%) yield: 1H NMR (500 MHz, DMSO-d₆) δ 8.18 (s, 1H), 8.00 (s, 1H), 7.95 (s, 1H), 7.88 (ddd, J=8.2, 2.3, 1.0 Hz, 1H), 7.40 (t, J=2.0 Hz, 1H), 7.35 (t, J=8.0 Hz, 1H), 6.77 (dt, J=7.9, 1.1 Hz, 1H), 5.10 (s, 2H), 4.96 (d, J=6.0 Hz, 2H), 4.89 (d, J=6.0 Hz, 2H), 3.86 (s, 2H), 3.51 (s, 2H), 2.92 (t, J=6.5 Hz, 2H), 2.91 (s, 3H), 2.75 (t, J=6.9 Hz, 2H), 2.29 (tt, J=14.9, 6.9 Hz, 2H); LCMS: C₂₇H₂₆F₃N₅O₂ requires: 547, found: m/z=548 [M+H]⁺.

### Example 412 and Example 413: 6-((S)-1-((S)-3-fluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one (412) and 6-((R)-1-((S)-3-fluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one (413)

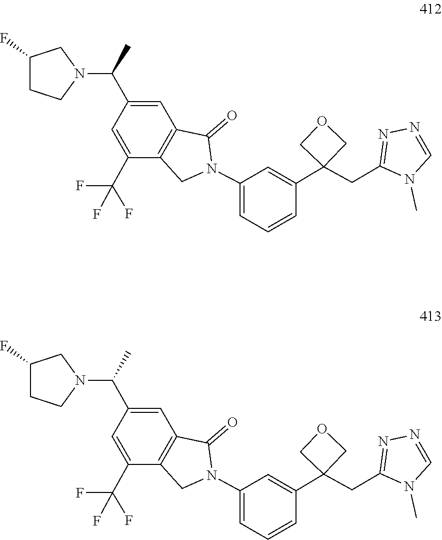 [SMILES: C[C@@H](c1cc2c(c(C(F)(F)F)c1)CN(c1cccc(C3(Cc4nncn4C)COC3)c1)C2=O)N1CC[C@H](F)C1 ; C[C@H](c1cc2c(c(C(F)(F)F)c1)CN(c1cccc(C3(Cc4nncn4C)COC3)c1)C2=O)N1CC[C@H](F)C1]

Step 1: Synthesis of 6-acetyl-2-(3-(3-((4-methyl-4H-1,2,4-triazol-3-yl)methyl)oxetan-3-yl)phenyl)-4-(trifluoromethyl)isoindolin-1-one. The indolone formation reaction was carried out in a manner similar to 260, step 2 using 3-(3-((4-methyl-4H-1,2,4-triazol-3-yl)methyl)oxetan-3-yl)aniline (3.18 g, 13.0 mmol, 1.0 eq.) and methyl 5-acetyl-2-(bromomethyl)-3-(trifluoromethyl)benzoate (4.41 g, 13.0 mmol, 1.0 eq.) as reactants to afford the title compound (4.96 g, 81%) as a yellow solid.

Step 2: Synthesis of 6-(1-((S)-3-fluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one. Reductive amination of the methyl ketone was carried out according to the procedure for 459, step 2 using 6-acetyl-2-(3-(3-((4-methyl-4H-1,2,4-triazol-3-yl)methyl)oxetan-3-yl)phenyl)-4-(trifluoromethyl)isoindolin-1-one (41 mg, 0.088 mmol, 1.0 eq.) and (S)-3-fluoropyrrolidine hydrochloride (63 mg, 0.44 mmol, 5.0 eq.) as reactants to afford the title compound (25 mg, 51%) as a colorless solid.

Step 3: Synthesis of 6-((S)-1-((S)-3-fluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one and 6-((R)-1-((S)-3-fluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one. The diastereomers of 6-(1-((S)-3-fluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one (25 mg) were separated using IG column with CO₂ and methanol as mobile phase to afford).

6-((S)-1-((S)-3-fluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one: (7.8 mg)¹H NMR (500 MHz, DMSO-d6) δ 8.17 (s, 1H), 7.99 (s, 1H), 7.94 (s, 1H), 7.87 (dd, J=8.1, 2.2 Hz, 1H), 7.39-7.29 (m, 2H), 6.79-6.72 (m, 1H), 5.28-5.05 (m, 3H), 4.95 (d, J=6.0 Hz, 2H), 4.88 (d, J=6.1 Hz, 2H), 3.59 (q, J=6.5 Hz, 1H), 3.50 (s, 2H), 2.94-2.90 (m, 1H), 2.88 (s, 3H), 2.59 (dd, J=30.2, 4.3 Hz, 2H), 2.28-2.06 (m, 2H), 1.90 (ddt, J=30.2, 14.3, 6.9 Hz, 1H), 1.36 (d, J=6.6 Hz, 3H); LCMS: C₂₈H₂₉F₄N₅O₂ requires: 543, found: m/z=544 [M+H]⁺.

6-((R)-1-((S)-3-Fluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one: (9.2 mg)¹H NMR (500 MHz, DMSO-d6) δ 8.17 (s, 1H), 8.00 (s, 1H), 7.94 (s, 1H), 7.87 (ddd, J=8.3, 2.3, 0.9 Hz, 1H), 7.39-7.31 (m, 2H), 6.76 (dt, J=7.7, 1.3 Hz, 1H), 5.30-5.11 (m, 1H), 5.11-5.06 (m, 2H), 4.95 (d, J=6.0 Hz, 2H), 4.88 (d, J=6.0 Hz, 2H), 3.62 (q, J=6.5 Hz, 1H), 3.50 (s, 2H), 2.92-2.86 (m, 1H), 2.89 (s, 3H), 2.71-2.54 (m, 2H), 2.43-2.32 (m, 1H), 2.11 (ddt, J=28.9, 14.0, 6.9 Hz, 1H), 1.96-1.79 (m, 1H), 1.35 (d, J=6.6 Hz, 3H); LCMS: C₂₈H₂₉F₄N₅O₂ requires: 543, found: m/z=544 [M+H]⁺.

### Example 414: 6-((cis-3,4-Difluoropyrrolidin-1-yl)methyl)-2-(3-(3-((4-methyl-4H-1,2,4-triazol-3-yl)methyl)oxetan-3-yl)phenyl)-4-(trifluoromethyl)isoindolin-1-one (414)

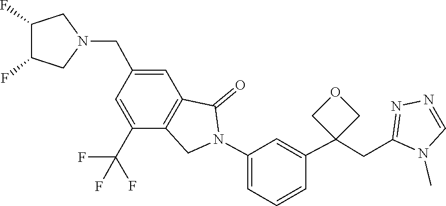 [SMILES: Cn1cnnc1CC1(c2cccc(N3Cc4c(cc(CN5C[C@@H](F)[C@@H](F)C5)cc4C(F)(F)F)C3=O)c2)COC1]

The reductive amination was carried out in a similar fashion as for 447, step 4 using Example Z (29 mg, 0.063 mmol, 1.0 eq.) and cis-3,4-difluoropyrrolidine hydrochloride (28 mg, 0.19 mmol, 3.1 eq.) as reactants to afford the title compound (13 mg, 37% yield) as a colorless solid. ¹H NMR (500 MHz, Methanol-d4) δ 8.18 (s, 1H), 8.05 (s, 1H), 7.96 (d, J=1.3 Hz, 1H), 7.75 (ddd, J=8.2, 2.3, 0.9 Hz, 1H), 7.42-7.35 (m, 2H), 6.74 (ddd, J=7.6, 1.8, 0.9 Hz, 1H), 5.20-4.97 (m, 7H), 3.90 (s, 2H), 3.65 (s, 2H), 3.05-2.90 (m, 3H), 2.89 (s, 3H); LCMS: C₂₇H₂₆F₅N₅O₂ requires: 547, found: m/z=548 [M+H]⁺.

### Example 415: 6-((3-fluoroazetidin-1-yl)methyl)-2-(3-(3-((4-methyl-4H-1,2,4-triazol-3-yl)methyl)oxetan-3-yl)phenyl)-4-(trifluoromethyl)isoindolin-1-one (415)

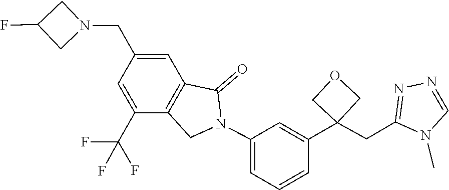 [SMILES: Cn1cnnc1CC1(c2cccc(N3Cc4c(cc(CN5CC(F)C5)cc4C(F)(F)F)C3=O)c2)COC1]

The reductive amination was carried out in a similar fashion as for 411, step 2 using Example Z (32 mg, 0.070 mmol, 1.0 eq.) and 3-fluoroazetidine hydrochloride (23 mg, 0.21 mmol, 3.0 eq.) as reactants to afford the title compound (23 mg, 64% yield) as a colorless solid. ¹H NMR (500 MHz, DMSO-d6) δ 8.17 (s, 1H), 7.95 (s, 1H), 7.90 (s, 1H), 7.87 (ddd, J=8.2, 2.2, 0.9 Hz, 1H), 7.38 (t, J=2.0 Hz, 1H), 7.34 (t, J=8.0 Hz, 1H), 6.75 (dt, J=7.7, 1.3 Hz, 1H), 5.20 (dt, J=57.7, 5.1 Hz, 1H), 5.08 (s, 2H), 4.95 (d, J=6.1 Hz, 2H), 4.87 (d, J=6.0 Hz, 2H), 3.84 (s, 2H), 3.58 (dt, J=15.5, 7.3 Hz, 2H), 3.50 (s, 2H), 3.25-3.14 (m, 1H), 2.89 (s, 3H); LCMS: C₂₆H₂₅F₄N₅O₂ requires: 515, found: m/z=516 [M+H]⁺.

### Example 416: 6-(1-((S)-3-hydroxypyrrolidin-1-yl)ethyl)-2-(3-((R)-1-(4-methyl-4H-1,2,4-triazol-3-yl)propan-2-yl)phenyl)-4-(trifluoromethyl)isoindolin-1-one (416)

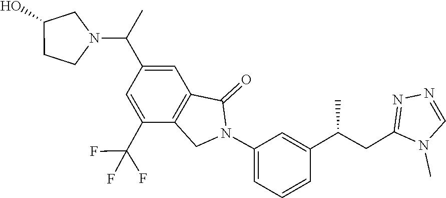 [SMILES: CC(c1cc2c(c(C(F)(F)F)c1)CN(c1cccc([C@H](C)Cc3nncn3C)c1)C2=O)N1CC[C@H](O)C1]

Reductive amination of the methyl ketone was carried out according to the procedure for 459, step 2 using (R)-6-acetyl-2-(3-(1-(4-methyl-4H-1,2,4-triazol-3-yl)propan-2-yl)phenyl)-4-(trifluoromethyl)isoindolin-1-one (200 mg, 0.21 mmol, 1.0 eq.) and (S)-pyrrolidin-3-ol (26 mg, 0.29 mmol, 1.4 eq.) as reactants to afford the title compound (3.5 mg, 3% yield) as a colorless solid: ¹H NMR (500 MHz, DMSO-d6) δ 8.27 (s, 1H), 8.02-7.97 (m, 1H), 7.92 (s, 1H), 7.82-7.74 (m, 2H), 7.35 (t, J=7.8 Hz, 1H), 7.10 (d, J=7.7 Hz, 1H), 5.16 (s, 2H), 4.68 (dd, J=4.5, 2.1 Hz, 1H), 4.17 (s, 1H), 3.58-3.47 (m, 1H), 3.44 (s, 3H), 3.00 (dd, J=7.4, 3.1 Hz, 2H), 2.77-2.62 (m, 1H), 2.30-2.19 (m, 1H), 1.98 (dt, J=13.3, 6.5 Hz, 1H), 1.61-1.49 (m, 1H), 1.39-1.26 (m, 6H); LCMS: C₂₇H₃₀F₃N₅O₂ requires: 513, found: m/z=514 [M+H]⁺.

### Example 417: 6-((3,3-difluoro-4-hydroxypyrrolidin-1-yl)methyl)-2-(3-((R)-1-(4-methyl-4H-1,2,4-triazol-3-yl)propan-2-yl)phenyl)-4-(trifluoromethyl)isoindolin-1-one (417)

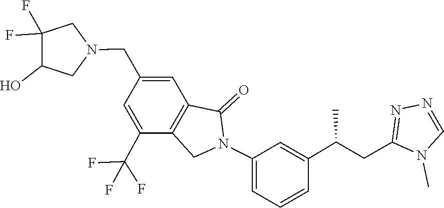 [SMILES: C[C@H](Cc1nncn1C)c1cccc(N2Cc3c(cc(CN4CC(O)C(F)(F)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (27 mg, 0.062 mmol, 1.0 eq.) and 4,4-difluoropyrrolidin-3-ol hydrochloride (28 mg, 0.18 mmol, 2.9 eq.) as reactants to afford the title compound (19 mg, 47% yield) as a colorless solid. ¹H NMR (500 MHz, DMSO-d6) δ 8.28 (s, 1H), 8.01 (s, 1H), 7.94 (s, 1H), 7.84-7.76 (m, 2H), 7.37 (dd, J=8.8, 7.6 Hz, 1H), 7.16-7.09 (m, 1H), 5.74 (d, J=5.8 Hz, 1H), 5.19 (s, 2H), 4.11 (dd, J=13.4, 8.1 Hz, 1H), 3.85 (q, J=13.6 Hz, 2H), 3.46 (s, 3H), 3.14 (ddd, J=22.2, 8.3, 5.3 Hz, 2H), 3.07-2.95 (m, 2H), 2.78 (ddd, J=17.7, 14.9, 11.2 Hz, 1H), 2.43-2.35 (m, 2H), 1.32 (d, J=6.9 Hz, 3H); LCMS: C₂₆H₂₆F₃N₅O₂ requires: 535, found: m/z=536 [M+H]⁺.

### Example 418: 6-((cis-3-fluoro-4-hydroxypyrrolidin-1-yl(methyl)-2-(3-((R)-1-(4-methyl-4H-1,2,4-triazol-3-yl)propan-2-yl)phenyl)-4-(trifluoromethyl)isoindolin-1-one (418)

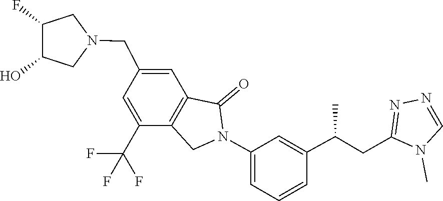 [SMILES: C[C@H](Cc1nncn1C)c1cccc(N2Cc3c(cc(CN4C[C@@H](F)[C@@H](O)C4)cc3C(F)(F)F)C2=O)c1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (50 mg, 0.12 mmol, 1 eq.) and cis-4-fluoropyrrolidin-3-ol hydrochloride (44 mg, 0.31 mmol, 2.7 eq.) as reactants to afford the title compound (26 mg, 43% yield) as a colorless solid. 1H NMR (500 MHz, DMSO-d6) δ 8.27 (s, 1H), 7.98 (s, 1H), 7.91 (s, 1H), 7.82-7.75 (m, 2H), 7.38-7.31 (m, 1H), 7.10 (d, J=7.6 Hz, 1H), 5.17 (s, 2H), 5.10 (d, J=6.5 Hz, 1H), 4.99-4.76 (m, 1H), 4.10 (dq, J=19.3, 6.7 Hz, 1H), 3.91-3.79 (m, 2H), 3.44 (s, 3H), 3.14-2.94 (m, 3H), 2.85 (dd, J=9.0, 6.9 Hz, 1H), 2.77-2.60 (m, 1H), 1.30 (d, J=6.9 Hz, 3H); LCMS: C₂₆H₂₇F₄N₅O₂ requires: 517, found: m/z=518 [M+H]⁺.

### Example 419 and Example 420: (R)-6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one (419) and (S)-6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one (420)

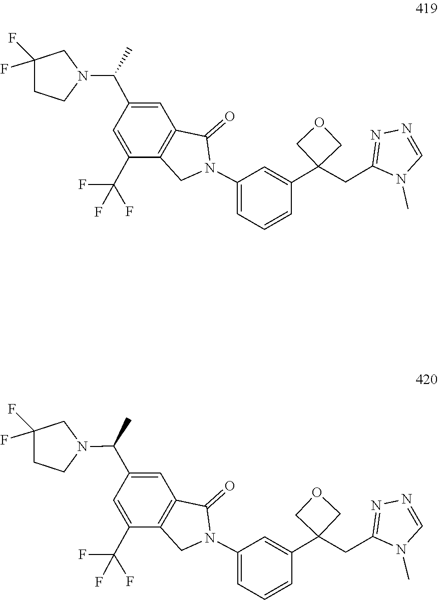 [SMILES: C[C@H](c1cc2c(c(C(F)(F)F)c1)CN(c1cccc(C3(Cc4nncn4C)COC3)c1)C2=O)N1CCC(F)(F)C1 ; C[C@@H](c1cc2c(c(C(F)(F)F)c1)CN(c1cccc(C3(Cc4nncn4C)COC3)c1)C2=O)N1CCC(F)(F)C1]

Step 1: Synthesis of 6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one. Reductive amination of the methyl ketone was carried out according to the procedure for 459, step 2 using 6-acetyl-2-(3-(3-((4-methyl-4H-1,2,4-triazol-3-yl)methyl)oxetan-3-yl)phenyl)-4-(trifluoromethyl)isoindolin-1-one (41 mg, 0.088 mmol, 1.0 eq.) and 3,3-difluoropyrrolidine hydrochloride (63 mg, 0.44 mmol, 5.0 eq.) as reactants to afford the title compound (25 mg, 51% yield) as a colorless solid.

Step 2: Synthesis of (R)-6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one and (S)-6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one. The diastereomers of 6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one (24 mg) were separated using IG column with CO₂ and methanol as mobile phase to afford:

(R)-6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one: (7.3 mg)¹H NMR (500 MHz, DMSO-d6) δ 8.17 (s, 1H), 8.00 (s, 1H), 7.94 (s, 1H), 7.87 (ddd, J=8.3, 2.3, 0.9 Hz, 1H), 7.40-7.31 (m, 2H), 6.76 (dt, J=7.7, 1.3 Hz, 1H), 5.08 (s, 2H), 4.95 (d, J=6.0 Hz, 2H), 4.88 (d, J=6.1 Hz, 2H), 3.70 (q, J=6.6 Hz, 1H), 3.50 (s, 2H), 2.97 (q, J=12.8 Hz, 1H), 2.89 (s, 3H), 2.84-2.69 (m, 1H), 2.25 (tt, J=14.9, 7.0 Hz, 2H), 1.35 (d, J=6.6 Hz, 3H); LCMS: C₂₈H₂₈F₅N₅O₂ requires: 561, found: m/z=562 [M+H]⁺.

(S)-6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one: (7.5 mg)¹H NMR (500 MHz, DMSO-d6) δ 8.17 (s, 1H), 8.00 (s, 1H), 7.94 (s, 1H), 7.87 (ddd, J=8.2, 2.2, 0.9 Hz, 1H), 7.40-7.30 (m, 2H), 6.76 (dt, J=8.0, 1.1 Hz, 1H), 5.12-5.05 (m, 2H), 4.95 (d, J=6.0 Hz, 2H), 4.88 (d, J=6.1 Hz, 2H), 3.70 (q, J=6.6 Hz, 1H), 3.50 (s, 2H), 2.97 (q, J=12.7 Hz, 1H), 2.89 (s, 3H), 2.83-2.66 (m, 2H), 2.31-2.13 (m, 2H), 1.35 (d, J=6.5 Hz, 3H); LCMS: C₂₈H₂₈F₅N₅O₂ requires: 561, found: m/z=562 [M+H]⁺.

### Example 421: (R)-1-((2-(3-(3-((4-methyl-4H-1,2,4-triazol-3-yl)methyl)oxetan-3-yl)phenyl)-3-oxo-7-(trifluoromethyl)isoindolin-5-yl)methyl)pyrrolidine-3-carbonitrile (421)

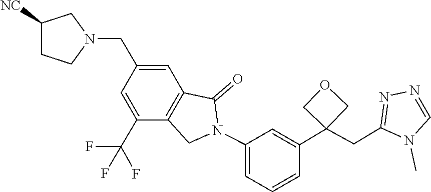 [SMILES: Cn1cnnc1CC1(c2cccc(N3Cc4c(cc(CN5CC[C@@H](C#N)C5)cc4C(F)(F)F)C3=O)c2)COC1]

The reductive amination was carried out in a similar fashion as for 447, step 4 using Example Z (30 mg, 0.065 mmol, 1.0 eq.) and (R)-pyrrolidine-3-carbonitrilehydrochloride (27 mg, 0.20 mmol, 3.1 eq.) as reactants to afford the title compound (21 mg, 59% yield) as a colorless solid. ¹H NMR (500 MHz, DMSO-d6) δ 8.17 (s, 1H), 7.99 (s, 1H), 7.94 (s, 1H), 7.87 (dd, J=8.1, 2.2 Hz, 1H), 7.39 (t, J=2.0 Hz, 1H), 7.34 (t, J=8.0 Hz, 1H), 6.75 (d, J=7.8 Hz, 1H), 5.09 (s, 2H), 4.95 (d, J=6.0 Hz, 2H), 4.88 (d, J=6.0 Hz, 2H), 3.84 (s, 2H), 3.50 (s, 2H), 2.89 (s, 3H), 2.84-2.66 (m, 3H), 2.27-2.15 (m, 1H), 2.02-1.91 (m, 1H); LCMS: C₂₈H₂₇F₃N₅O₂ requires: 536, found: m/z=537 [M+H]⁺.

### Example 422: (S)-1-((2-(3-(3-((4-methyl-4H-1,2,4-triazol-3-yl)methyl)oxetan-3-yl)phenyl)-3-oxo-7-(trifluoromethyl)isoindolin-5-yl)methyl)pyrrolidine-3-carbonitrile (422)

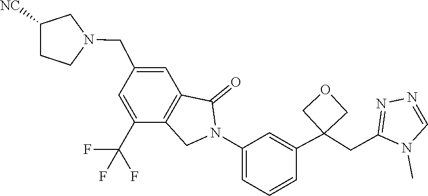 [SMILES: Cn1cnnc1CC1(c2cccc(N3Cc4c(cc(CN5CC[C@H](C#N)C5)cc4C(F)(F)F)C3=O)c2)COC1]

The reductive amination was carried out in a similar fashion as for 447, step 4 using Example Z (31 mg, 0.067 mmol, 1.0 eq.) and (S)-pyrrolidine-3-carbonitrile hydrochloride (28 mg, 0.21 mmol, 3.1 eq.) as reactants to afford the title compound (21 mg, 58% yield) as a colorless solid. ¹H NMR (500 MHz, DMSO-d6) δ 8.17 (s, 1H), 7.99 (s, 1H), 7.94 (s, 1H), 7.87 (ddd, J=8.3, 2.2, 0.9 Hz, 1H), 7.39 (t, J=2.0 Hz, 1H), 7.34 (t, J=7.9 Hz, 1H), 6.78-6.73 (m, 1H), 5.09 (s, 2H), 4.95 (d, J=6.0 Hz, 2H), 4.88 (d, J=6.0 Hz, 2H), 3.84 (s, 2H), 3.50 (s, 2H), 2.89 (s, 3H), 2.83-2.67 (m, 3H), 2.26-2.15 (m, 1H), 2.03-1.91 (m, 1H); LCMS: C₂₈H₂₇F₃N₆O₂ requires: 536, found: m/z=537 [M+H]⁺.

### Example 423: 1-((2-(3-(3-((4-methyl-4H-1,2,4-triazol-3-yl)methyl)oxetan-3-yl)phenyl)-3-oxo-7-(trifluoromethyl)isoindolin-5-yl)methyl)azetidine-3-carbonitrile (423)

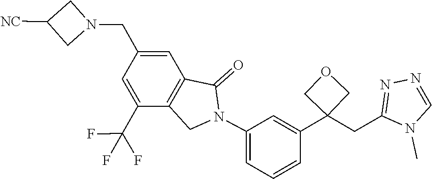 [SMILES: Cn1cnnc1CC1(c2cccc(N3Cc4c(cc(CN5CC(C#N)C5)cc4C(F)(F)F)C3=O)c2)COC1]

The reductive amination was carried out in a similar fashion as for 411, step 2 using Example Z (31 mg, 0.068 mmol, 1.0 eq.) and azetidine-3-carbonitrile hydrochloride (24 mg, 0.20 mmol, 3.0 eq.) as reactants to afford the title compound (15 mg, 42% yield) as a colorless solid. ¹H NMR (500 MHz, DMSO-d6) δ 8.17 (s, 1H), 7.94 (s, 1H), 7.89 (s, 1H), 7.87 (ddd, J=8.3, 2.3, 1.0 Hz, 1H), 7.38 (t, J=2.0 Hz, 1H), 7.34 (t, J=7.9 Hz, 1H), 6.75 (dt, J=7.6, 1.3 Hz, 1H), 5.08 (s, 2H), 4.95 (d, J=6.0 Hz, 2H), 4.87 (d, J=6.1 Hz, 2H), 3.79 (s, 2H), 3.60-3.44 (m, 5H), 2.89 (s, 3H); LCMS: C₂₇H₂₅F₃N₆O₂ requires: 522, found: m/z=523 [M+H]⁺.

### Example 424: 6-((3-fluoro-3-methylazetidin-1-yl)methyl)-2-(3-(3-((4-methyl-4H-1,2,4-triazol-3-yl)methyl)oxetan-3-yl)phenyl)-4-(trifluoromethyl)isoindolin-1-one (424)

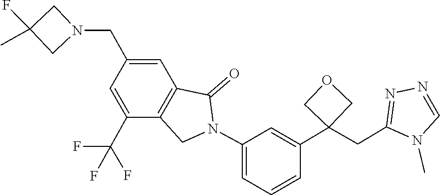 [SMILES: Cn1cnnc1CC1(c2cccc(N3Cc4c(cc(CN5CC(C)(F)C5)cc4C(F)(F)F)C3=O)c2)COC1]

The reductive amination was carried out in a similar fashion as for 411, step 2 using Example Z (30 mg, 0.065 mmol, 1.0 eq.) and 3-fluoro-3-methylazetidine hydrochloride (44 mg, 0.35 mmol, 5.5 eq.) as reactants to afford the title compound (16 mg, 47% yield) as a colorless solid. ¹H NMR (500 MHz, DMSO-d6) δ 8.17 (s, 1H), 7.95 (s, 1H), 7.90 (s, 1H), 7.86 (ddd, J=8.2, 2.2, 1.0 Hz, 1H), 7.39 (t, J=2.0 Hz, 1H), 7.34 (t, J=8.0 Hz, 1H), 6.79-6.73 (m, 1H), 5.08 (s, 2H), 4.95 (d, J=6.0 Hz, 2H), 4.88 (d, J=6.1 Hz, 2H), 3.85 (s, 2H), 3.50 (s, 2H), 2.89 (s, 3H), 1.54 (d, J=22.4 Hz, 3H); LCMS: requires: 529, found: m/z=530 [M+H]⁺.

### Example 425: 3-methyl-1-((2-(3-(3-((4-methyl-4H-1,2,4-triazol-3-yl)methyl)oxetan-3-yl)phenyl)-3-oxo-7-(trifluoromethyl)isoindolin-5-yl)methyl)azetidine-3-carbonitrile (425)

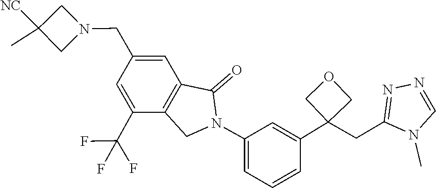 [SMILES: Cn1cnnc1CC1(c2cccc(N3Cc4c(cc(CN5CC(C)(C#N)C5)cc4C(F)(F)F)C3=O)c2)COC1]

The reductive amination was carried out in a similar fashion as for 411, step 2 using Example Z (30 mg, 0.065 mmol, 1.0 eq.) and 3-methylazetidine-3-carbonitrile hydrochloride (47 mg, 0.35 mmol, 5.4 eq.) as reactants to afford the title compound (14 mg, 39% yield) as a colorless solid. ¹H NMR (500 MHz, DMSO-d6) δ 8.17 (s, 1H), 7.94 (d, J=1.4 Hz, 1H), 7.90 (s, 1H), 7.87 (ddd, J=8.2, 2.2, 1.0 Hz, 1H), 7.39 (t, J=2.0 Hz, 1H), 7.34 (t, J=7.9 Hz, 1H), 6.75 (dt, J=7.9, 1.2 Hz, 1H), 5.09 (s, 2H), 4.95 (d, J=6.1 Hz, 2H), 4.88 (d, J=6.1 Hz, 2H), 3.81 (s, 2H), 3.58-3.44 (m, 4H), 3.20 (d, J=7.1 Hz, 2H), 2.89 (s, 3H), 1.55 (s, 3H); LCMS: C₂₈H₂₇F₃N₅O₂ requires: 536, found: m/z=537 [M+H]⁺.

### Example 426: 2-(3-(3-((4-methyl-4H-1,2,4-triazol-3-yl)methyl)oxetan-3-yl)phenyl)-6-((2-oxopyrrolidin-1-yl)methyl)-4-(trifluoromethyl)isoindolin-1-one (426)

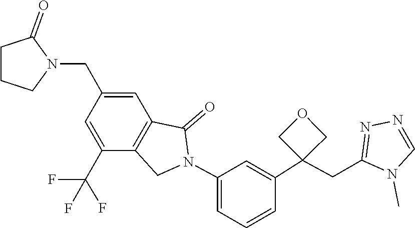 [SMILES: Cn1cnnc1CC1(c2cccc(N3Cc4c(cc(CN5CCCC5=O)cc4C(F)(F)F)C3=O)c2)COC1]

The reductive amination was carried out in a similar fashion as for 411, step 2 using Example Z (30 mg, 0.067 mmol, 1.0 eq.) and methyl 5-aminopentanoate hydrochloride (54 mg, 0.35 mmol, 5.3 eq.) as reactants to afford the title compound (19 mg, 54% yield) as a colorless solid. ¹H NMR (500 MHz, DMSO-d6) δ 8.17 (s, 1H), 7.91-7.82 (m, 3H), 7.38 (t, J=2.0 Hz, 1H), 7.34 (t, J=8.0 Hz, 1H), 6.76 (dt, J=7.8, 1.1 Hz, 1H), 5.10 (s, 2H), 4.95 (d, J=6.0 Hz, 2H), 4.87 (d, J=6.0 Hz, 2H), 4.58 (s, 2H), 3.50 (s, 2H), 2.89 (s, 3H), 2.32 (t, J=8.1 Hz, 2H), 1.94 (p, J=7.5 Hz, 2H); LCMS: C₂₇H₂₆F₃N₅O₃ requires: 525, found: m/z=526 [M+H]⁺.

### Example 427: (S)-6-((2-methyl-2,5-dihydro-1H-pyrrol-1-yl)methyl)-2-(3-(3-((4-methyl-4H-1,2,4-triazol-3-yl(methyl)oxetan-3-yl(phenyl)-4-(trifluoromethyl)isoindolin-1-one (427)

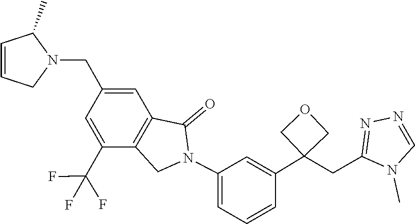 [SMILES: C[C@H]1C=CCN1Cc1cc2c(c(C(F)(F)F)c1)CN(c1cccc(C3(Cc4nncn4C)COC3)c1)C2=O]

Step 1: Synthesis of tert-butyl (S)-2-methyl-2,5-dihydro-1H-pyrrole-1-carboxylate. tert-Butyl (2S,3R)-3-hydroxy-2-methylpyrrolidine-1-carboxylate (101 mg, 0.502 mmol, 1.0 eq.) was dissolved in dichloromethane (2 mL) and cooled to 0° C. Bis(2-methoxyethyl)aminosulfur trifluoride (2.7 M in toluene, 280 μL, 0.76 mmol, 1.5 eq.) was added and the reaction was gradually warmed to ambient temperature and stirred overnight. Sat. sodium bicarbonate solution was added, and the product was extracted with dichloromethane (×3). After drying with anhydrous sodium sulfate and removal of solvent under reduced pressure, the crude material was purified with silica gel column chromatography using a gradient of EtOAc in dichloromethane (0-20%) to afford the desired product in 51 mg yield.

Step 2: Synthesis of (S)-2-methyl-2,5-dihydro-1H-pyrrole hydrochloride, tert-Butyl (S)-2-methyl-2,5-dihydro-1H-pyrrole-1-carboxylate (49 mg, 0.27 mmol, 1.0 eq.) was dissolved in dichloromethane (0.5 mL) at rt. 4M-Hydrochloric acid in dioxane (0.5 mL) was added and the reaction stirred for 1 hour. After removal of solvents under reduced pressure, the material was lyophilized to obtain the title compound in 26 mg yield.

Step 3: Synthesis of (S)-6-((2-methyl-2,5-dihydro-1H-pyrrol-1-yl)methyl)-2-(3-(3-((4-methyl-4H-1,2,4-triazol-3-yl)methyl)oxetan-3-yl)phenyl)-4-(trifluoromethyl)isoindolin-1-one. The reductive amination was carried out in a similar fashion as for 447, step 4 using Example Z (25 mg, 0.10 mmol, 1.0 eq.) and (S)-2-methyl-2,5-dihydro-1H-pyrrole hydrochloride (26 mg, 0.22 mmol, 2.2 eq.) as reactants to afford the title compound (18 mg, 32% yield) as a colorless solid. ¹H NMR (500 MHz, DMSO-d6) δ 8.18 (s, 1H), 7.99 (s, 1H), 7.93 (s, 1H), 7.88 (dd, J=8.0, 2.1 Hz, 1H), 7.38 (t, J=2.0 Hz, 1H), 7.34 (t, J=7.9 Hz, 1H), 6.75 (d, J=7.7 Hz, 1H), 5.78-5.71 (m, 2H), 5.09 (s, 2H), 4.95 (d, J=6.0 Hz, 2H), 4.88 (d, J=6.1 Hz, 2H), 4.16 (d, J=14.0 Hz, 1H), 3.78 (d, J=14.1 Hz, 1H), 3.72-3.61 (m, 1H), 3.57-3.45 (m, 2H), 3.24-3.11 (m, 2H), 2.89 (s, 3H), 1.14 (d, J=6.4 Hz, 3H); LCMS: C₂₈H₂₈F₃N₅O₂ requires: 523, found: m/z=524 [M+H]⁺.

### Example 428: 1-((2-(3-(3-((4-methyl-4H-1,2,4-triazol-3-yl)methyl)oxetan-3-yl(phenyl)-3-oxo-7-(trifluoromethyl)isoindolin-5-yl)methyl)azetidine-3-carboxylic acid (428)

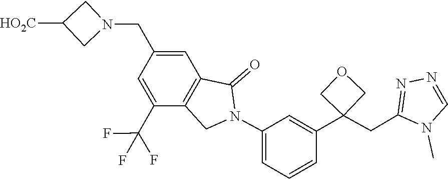 [SMILES: Cn1cnnc1CC1(c2cccc(N3Cc4c(cc(CN5CC(C(=O)O)C5)cc4C(F)(F)F)C3=O)c2)COC1]

The reductive amination was carried out in a similar fashion as for 447, step 4 using Example Z (29 mg, 0.064 mmol, 1.0 eq.) and azetidine-3-carboxylic acid (31 mg, 0.30 mmol, 4.7 eq.) as reactants to afford the title compound (15 mg, 34% yield) as a trifluoroacetate salt, ¹H NMR (500 MHz, DMSO-d6) δ 10.41 (s, 1H), 8.36 (s, 1H), 8.24 (s, 1H), 8.18 (d, J=12.4 Hz, 1H), 7.87 (dd, J=8.0, 2.2 Hz, 1H), 7.42 (t, J=1.9 Hz, 1H), 7.37 (t, J=8.0 Hz, 1H), 6.83 (d, J=7.7 Hz, 1H), 5.16 (s, 2H), 4.95 (d, J=6.1 Hz, 2H), 4.88 (d, J=6.1 Hz, 2H), 4.60 (s, 2H), 4.31 (s, 2H), 4.17 (s, 2H), 3.62 (d, J=9.4 Hz, 1H), 3.55 (s, 2H), 2.97 (s, 3H); LCMS: C₂₇H26F₃N₅O₄ requires: 541, found: m/z=542 [M+H]⁺.

### Example 429: 6-(((1R,5S)-3-azabicyclo[3.1.0]hexan-3-yl)methyl)-2-(3-(3-((4-methyl-4H-1,2,4-triazol-3-yl)methyl)oxetan-3-yl)phenyl)-4-(trifluoromethyl)isoindolin-1-one (429)

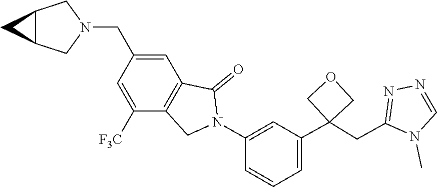 [SMILES: Cn1cnnc1CC1(c2cccc(N3Cc4c(cc(CN5C[C@H]6C[C@H]6C5)cc4C(F)(F)F)C3=O)c2)COC1]

The reductive amination was carried out in a similar fashion as for 447, step 4 using Example Z (50 mg, 0.1 mmol, 1 eq) and 3-azabicyclo[3.1.0]hexane hydrochloride (65 mg, 0.55 mmol, 5 eq) to afford the title compound as an off-white solid: ¹H NMR (500 MHz, DMSO-d₆) δ 8.18 (s, 1H), 7.92 (s, 1H), 7.90-7.82 (m, 2H), 7.40 (s, 1H), 7.35 (t, J=8.0 Hz, 1H), 6.76 (d, J=7.7 Hz, 1H), 5.09 (s, 2H), 4.96 (d, J=6.0 Hz, 2H), 4.89 (d, J=6.1 Hz, 2H), 3.79 (s, 2H), 3.51 (s, 2H), 2.95-2.85 (m, 4H), 2.37 (d, J=7.0 Hz, 2H), 1.38 (s, 2H), 0.68 (q, J=3.9 Hz, 1H), 0.40-0.31 (m, 1H); LCMS: C₂₈H₂₈F₃N₅O₂ requires: 523, found: m/z=524 [M+H]⁺.

### Example 430: 2-(3-(3-((4-methyl-4H-1,2,4-triazol-3-yl)methyl)oxetan-3-yl)phenyl)-6-(l-(3-(methylsulfonyl)azetidin-1-yl)ethyl)-4-(trifluoromethyl)isoindolin-1-one (430)

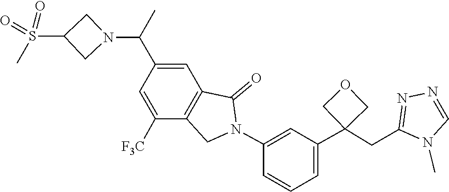 [SMILES: CC(c1cc2c(c(C(F)(F)F)c1)CN(c1cccc(C3(Cc4nncn4C)COC3)c1)C2=O)N1CC(S(C)(=O)=O)C1]

Reductive animation of the methyl ketone was carried out according to the procedure for 459, step 2 using 6-acetyl-2-(3-(3-((4-methyl-4H-1,2,4-triazol-3-yl)methyl)oxetan-3-yl)phenyl)-4-(trifluoromethyl)isoindolin-1-one (70 mg, 0.15 mmol) and 3-methanesulfonylazetidine (80 mg, 0.60 mmol) as reactants afford the title compound (8.0 mg, 9%) as an off-white solid: 1H NMR (500 MHz, DMSO-d6) δ 8.11 (s, 1H), 7.89 (d, J=23.9 Hz, 2H), 7.81 (ddd, J=8.3, 2.3, 1.0 Hz, 1H), 7.34-7.24 (m, 2H), 6.70 (dt, J=7.7, 1.1 Hz, 1H), 5.02 (s, 2H), 4.89 (d, J=6.1 Hz, 2H), 4.82 (d, J=6.1 Hz, 2H), 4.09 (p, J=7.4 Hz, 2H), 3.63 (q, J=6.5 Hz, 1H), 3.50 (t, J=8.1 Hz, 1H), 3.44 (s, 2H), 3.36-3.28 (m, 2H), 2.88 (s, 3H), 2.83 (s, 3H), 1.11 (d, J=6.4 Hz, 3H); LCMS: C₂₈H₃₀F₃N₅O₄S requires: 589, found: m/z=590 [M+H]+.

### Example 431: 2-(3-{3-[(4-methyl-4H-1,2,4-triazol-3-yl)methyl]oxetan-3-yl}phenyl)-6-[(morpholin-4-yl)methyl]-4-(trifluoromethyl)-2,3-dihydro-1H-isoindol-1-one (431)

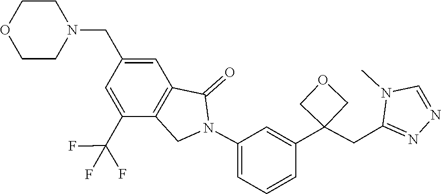 [SMILES: Cn1cnnc1CC1(c2cccc(N3Cc4c(cc(CN5CCOCC5)cc4C(F)(F)F)C3=O)c2)COC1]

The reductive amination was carried out in a similar fashion as for 447, step 4 using 2-(3-{3-[(4-methyl-4H-1,2,4-triazol-3-yl)methyl]oxetan-3-yl}phenyl)-3-oxo-7-(trifluoromethyl)-2,3-dihydro-1H-isoindole-5-carbaldehyde (40 mg, 0.09 mmol, 1.0 eq.) and morpholine (30 mg, 0.35 mmol, 3.9 eq.) as reactants to afford the title compound (9 mg, 20%) as an off-white solid: ¹H NMR (500 MHz, DMSO-d6) δ 8.19 (s, 1H), 8.01 (s, 1H), 7.96 (s, 1H), 7.93-7.86 (m, 1H), 7.41 (t, J=2.0 Hz, 1H), 7.36 (t, J=7.9 Hz, 1H), 6.82-6.74 (m, 1H), 5.11 (s, 2H), 4.97 (d, J=6.1 Hz, 2H), 4.90 (d, J=6.1 Hz, 2H), 3.70 (s, 2H), 3.61 (t, J=4.6 Hz, 4H), 3.52 (s, 2H), 2.91 (s, 3H), 2.45-2.36 (m, 4H); LCMS: C₂₇H₂₈F₃N₅O₃ requires: 527, found: m/z=528 [M+H]⁺.

### Example 432: 6-{[(3S)-3-fluoropiperidin-1-yl]methyl}-2-(3-{3-[(4-methyl-4H-1,2,4-triazol-3-yl)methyl]oxetan-3-yl}phenyl)-4-(trifluoromethyl)-2,3-dihydro-1H-isoindol-1-one (432)

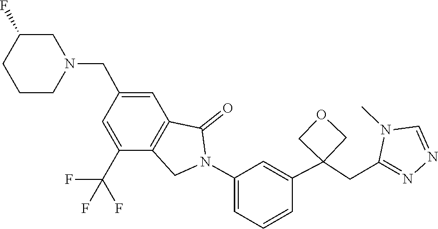 [SMILES: Cn1cnnc1CC1(c2cccc(N3Cc4c(cc(CN5CCC[C@H](F)C5)cc4C(F)(F)F)C3=O)c2)COC1]

The reductive amination was carried out in a similar fashion as for 447, step 4 using 2-(3-{3-[(4-methyl-4H-1,2,4-triazol-3-yl)methyl]oxetan-3-yl}phenyl)-3-oxo-7-(trifluoromethyl)-2,3-dihydro-1H-isoindole-5-carbaldehyde (50 mg, 0.11 mmol, 1.0 eq.) and (3S)-3-fluoropiperidine (45 mg, 0.44 mmol, 4.0 eq.) as reactants afford the title compound (11 mg, 19%) as an off-white solid: ¹H NMR (500 MHz, DMSO-d6) δ 8.20 (s, 1H), 8.00 (s, 1H), 7.94 (s, 1H), 7.89 (ddd, J=8.2, 2.3, 0.9 Hz, 1H), 7.40 (t, J=1.9 Hz, 1H), 7.36 (t, J=7.9 Hz, 1H), 6.77 (dt, J=7.9, 1.2 Hz, 1H), 5.11 (s, 2H), 4.94 (dd, J=38.5, 6.0 Hz, 4H), 4.67 (dt, J=48.2, 3.6 Hz, 1H), 3.75 (s, 2H), 3.52 (s, 2H), 3.30 (s, 3H), 2.70 (dd, J=20.8, 11.6 Hz, 1H), 2.48-2.35 (m, 3H), 1.78 (dt, J=32.9, 7.8 Hz, 2H), 1.63-1.44 (m, 2H); LCMS: C₂₈H₂₉F₄N₅O₂ requires: 543, found: m/z=544 [M+H]⁺.

### Example 433: 2-(3-{3-[(4-methyl-4H-1,2,4-triazol-3-yl)methyl]oxetan-3-yl}phenyl)-6-({2-oxa-5-azaspiro[3,4]octan-5-yl}methyl)-4-(trifluoromethyl)-2,3-dihydro-1H-isoindol-1-one (433)

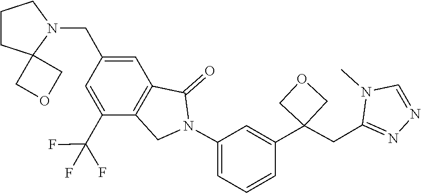 [SMILES: Cn1cnnc1CC1(c2cccc(N3Cc4c(cc(CN5CCCC56COC6)cc4C(F)(F)F)C3=O)c2)COC1]

The reductive amination was carried out in a similar fashion as for 447, step 4 using 2-(3-{3-[(4-methyl-4H-1,2,4-triazol-3-yl)methyl]oxetan-3-yl}phenyl)-3-oxo-7-(trifluoromethyl)-2,3-dihydro-1H-isoindole-5-carbaldehyde (30 mg, 0.07 mmol, 1.0 eq.) and bis(2-oxa-5-azaspiro[3,4]octane); oxalic acid (44 mg, 0.14 mmol, 2.0 eq.) as reactants afford the title compound (1 mg, 3%) as an off-white solid: ¹H NMR (500 MHz, DMSO-d6) δ 8.20 (s, 1H), 8.03 (s, 1H), 7.98 (s, 1H), 7.93-7.88 (m, 1H), 7.40 (t, J=2.0 Hz, 1H), 7.36 (t, J=7.9 Hz, 1H), 6.78 (dd, J=7.6, 1.2 Hz, 1H), 5.11 (s, 2H), 4.98 (d, J=6.0 Hz, 2H), 4.90 (d, J=6.0 Hz, 2H), 4.84 (d, J=6.6 Hz, 2H), 4.48 (d, J=6.7 Hz, 2H), 4.20 (s, 2H), 3.52 (s, 2H), 2.92 (s, 3H), 2.22-2.11 (m, 3H), 1.67 (p, J=7.2 Hz, 3H); LCMS: C₂₉H₃₀F₃N₅O₃ requires: 553, found: m/z=554 [M+H]⁺.

### Example 434: 4-{[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]methyl}-1λ⁶-thiomorpholine-1,1-dione (434)

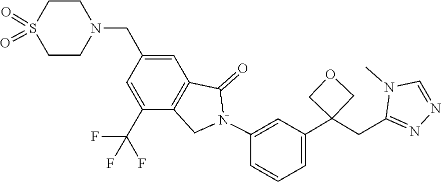 [SMILES: Cn1cnnc1CC1(c2cccc(N3Cc4c(cc(CN5CCS(=O)(=O)CC5)cc4C(F)(F)F)C3=O)c2)COC1]

The reductive amination was carried out in a similar fashion as for 447, step 4 using 2-(3-{3-[(4-methyl-4H-1,2,4-triazol-3-yl)methyl]oxetan-3-yl}phenyl)-3-oxo-7-(trifluoromethyl)-2,3-dihydro-1H-isoindole-5-carbaldehyde (50 mg, 0.11 mmol, 1.0 eq.) and thiomorpholine 1,1-dioxide (59 mg, 0.44 mmol, 4.0 eq.) as reactants afford the title compound (26 mg, 41%) as an off-white solid: ¹H NMR (500 MHz, DMSO-d₆) δ 8.19 (d, J=2.2 Hz, 1H), 8.00 (dd, J=26.6, 20.7 Hz, 2H), 7.89 (ddd, J=8.3, 2.3, 1.0 Hz, 1H), 7.44-7.30 (m, 2H), 6.77 (tt, J=7.9, 1.2 Hz, 1H), 5.59 (t, J=5.8 Hz, 1H), 5.11 (s, 2H), 4.97 (d, J=6.1 Hz, 2H), 4.89 (d, J=6.1 Hz, 2H), 4.71 (d, J=5.9 Hz, 2H), 3.91 (s, 2H), 3.52 (s, 3H), 3.14 (d, J=5.6 Hz, 2H), 2.97-2.89 (m, 5H); LCMS: C₂₇H₂₈F₃N₅O₄S requires: 575, found: m/z=576 [M+H]⁺.

### Example 435: 6-((3-fluoro-3-methylpyrrolidin-1-yl)methyl)-2-(3-(3-((4-methyl-4H-1,2,4-triazol-3-yl)methyl)oxetan-3-yl)phenyl)-4-(trifluoromethyl)isoindolin-1-one (435)

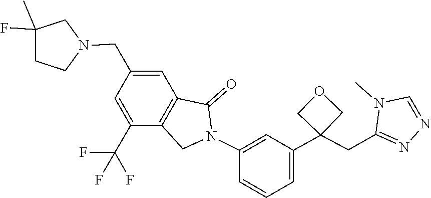 [SMILES: Cn1cnnc1CC1(c2cccc(N3Cc4c(cc(CN5CCC(C)(F)C5)cc4C(F)(F)F)C3=O)c2)COC1]

The reductive amination was carried out in a similar fashion as for 447, step 4 using 2-(3-{3-[(4-methyl-4H-1,2,4-triazol-3-yl)methyl]oxetan-3-yl}phenyl)-3-oxo-7-(trifluoromethyl)-2,3-dihydro-1H-isoindole-5-carbaldehyde (50 mg, 0.11 mmol) and 3-fluoro-3-methylpyrrolidine hydrochloride (61 mg, 0.44 mmol) as reactants afford the title compound (31 mg, 52%) as an off-white solid: ¹H NMR (500 MHz, DMSO-d6) δ 8.20 (s, 1H), 8.00 (s, 1H), 7.95 (s, 1H), 7.89 (dd, J=7.8, 2.1 Hz, 1H), 7.41 (t, J=2.0 Hz, 1H), 7.36 (t, J=7.9 Hz, 1H), 6.78 (dd, J=7.7, 1.3 Hz, 1H), 5.11 (s, 2H), 4.98 (d, J=6.1 Hz, 2H), 4.90 (d, J=6.1 Hz, 2H), 3.84 (s, 2H), 3.52 (s, 2H), 2.91 (s, 3H), 2.90-2.78 (m, 3H), 2.14-1.85 (m, 4H), 1.45 (d, J=21.3 Hz, 3H); LCMS: C₂₈H₂₉F₄N₅O₂ requires: 543, found: m/z=544 [M+H]+.

### Example 436: 6-(1-hydroxyethyl)-2-(3-((R)-1-(4-methyl-4H-1,2,4-triazol-3-yl)propan-2-yl)phenyl)-4-(trifluoromethyl)isoindolin-1-one (436)

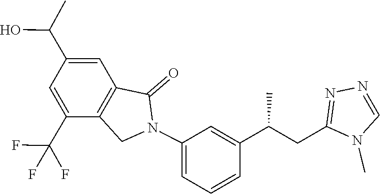 [SMILES: CC(O)c1cc2c(c(C(F)(F)F)c1)CN(c1cccc([C@H](C)Cc3nncn3C)c1)C2=O]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)isoindoline-5-carbaldehyde (24 mg, 0.055 mmol, 1.0 eq.) and azetidin-3-ol (21 mg, 0.28 mmol, 5.1 eq.) as reactants which afforded the title compound (1.7 mg, 7% yield) as a byproduct. ¹H NMR (500 MHz, DMSO-d6) δ 8.27 (s, 1H), 8.02 (s, 1H), 7.96 (s, 1H), 7.81-7.79 (m, 2H), 7.39-7.32 (m, 1H), 7.10 (d, J=7.7 Hz, 1H), 5.55 (d, J=4.6 Hz, 1H), 5.16 (s, 1H), 4.96 (p, J=6.4 Hz, 1H), 3.44 (s, 3H), 3.06-2.93 (m, 2H), 1.39 (d, J=6.4 Hz, 3H), 1.30 (d, J=6.9 Hz, 3H); LCMS: C₂₃H₂₃F₃N₄O₂ requires: 444, found: m/z=445 [M+H]⁺.

### Example 437: 6-(hydroxymethyl)-2-(3-(3-((4-methyl-4H-1,2,4-triazol-3-yl)methyl)oxetan-3-yl)phenyl)-4-(trifluoromethyl)isoindolin-1-one (437)

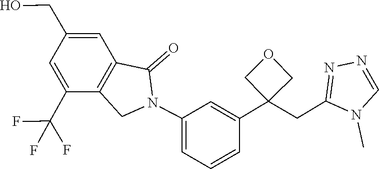 [SMILES: Cn1cnnc1CC1(c2cccc(N3Cc4c(cc(CO)cc4C(F)(F)F)C3=O)c2)COC1]

The reductive amination was carried out in a similar fashion as for 447, step 4 using Example Z (30 mg, 0.066 mmol, 1.0 eq.) and azetidine-3-carboxylic acid (22 mg, 0.21 mmol, 3.2 eq.) as reactants which afforded the title compound (6.4 mg, 21% yield) as a byproduct. ¹H NMR (500 MHz, DMSO-d6) δ 8.18 (s, 1H), 7.99 (s, 1H), 7.94 (s, 1H), 7.88 (ddd, J=8.3, 2.3, 0.9 Hz, 1H), 7.40 (t, J=2.0 Hz, 1H), 7.34 (t, J=8.0 Hz, 1H), 6.75 (dt, J=7.9, 1.1 Hz, 1H), 5.57 (t, J=5.8 Hz, 1H), 5.09 (s, 2H), 4.95 (d, J=6.0 Hz, 2H), 4.88 (d, J=6.0 Hz, 2H), 4.70 (d, J=5.7 Hz, 2H), 3.50 (s, 2H), 2.90 (s, 3H); LCMS: C₂₃H₂₁F₃N₄O₃ requires: 458, found: m/z=459 [M+H]⁺.

### Example 438: 6-((S)-3-hydroxypyrrolidine-1-carbonyl)-2-(3-((R)-1,1,2-trifluoro-1-(4-methyl-4H-1,2,4-triazol-3-yl)propan-2-yl)phenyl)-4-(trifluoromethyl)isoindolin-1-one (438)

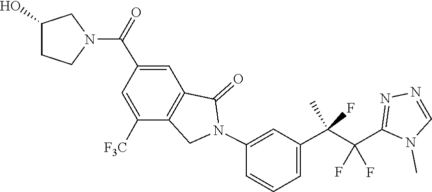 [SMILES: Cn1cnnc1C(F)(F)[C@](C)(F)c1cccc(N2Cc3c(cc(C(=O)N4CC[C@H](O)C4)cc3C(F)(F)F)C2=O)c1]

(R)-3-oxo-2-(3-(1,1,2-trifluoro-1-(4-methyl-4H-1,2,4-triazol-3-yl)propan-2-yl)phenyl)-7-(trifluoromethyl)isoindoline-5-carboxylic acid (130 mg, 0.26 mmol, 1.0 eq) and (S)-3-hydroxypyrrolidine (28 mg, 0.33 mmol, 1.25 eq) were coupled in a manner similar to 184 to afford the title compound (122 mg) as an off-white solid: ¹H NMR (500 MHz, DMSO-d₆) (mixture of retainers) δ 8.63 (s, 1H), 8.14 (d, J=2.6 Hz, 1H), 8.12 (s, 1H), 8.05 (d, J=1.9 Hz, 1H), 7.91 (ddd, J=8.3, 2.2, 0.9 Hz, 1H), 7.53 (t, J=8.0 Hz, 1H), 7.24 (dd, J=8.0, 1.6 Hz, 1H), 5.32-5.20 (m, 2H), 5.06 (d, J=4.2 Hz, 1H), 4.99 (d, J=3.6 Hz, 1H), 4.35 (t, J=4.6 Hz, 1H), 4.26 (s, 1H), 3.68-3.55 (m, 2H), 3.46 (s, 3H), 3.23-3.16 (m, 1H), 2.05-1.92 (m, 3H), 1.90-1.76 (m, 1H); LCMS: C₂₆H₂₃F₆N₅O₃ requires: 567, found: m/z=568 [M+H]⁺.

### Example 439: (R)-6-(3-(difluoromethyl)azetidine-1-carbonyl)-2-(3-(1,1,2-trifluoro-1-(4-methyl-4H-1,2,4-triazol-3-yl)propan-2-yl)phenyl)-4-(trifluoromethyl)isoindolin-1-one (439)

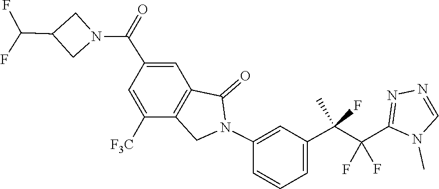 [SMILES: Cn1cnnc1C(F)(F)[C@](C)(F)c1cccc(N2Cc3c(cc(C(=O)N4CC(C(F)F)C4)cc3C(F)(F)F)C2=O)c1]

(R)-3-oxo-2-(3-(1,1,2-trifluoro-1-(4-methyl-4H-1,2,4-triazol-3-yl)propan-2-yl)-phenyl)-7-(trifluoromethyl)isoindoline-5-carboxylic acid (50 mg, 0.1 mmol, 1 eq) and 3-(difluoromethyl)azetidine hydrochloride (15 mg, 0.11 mmol, 1.1 eq) were coupled in a manner similar to 184 to afford 22 mg of the title compound as an off-white solid: ¹H NMR (500 MHz, DMSO-d₆) δ 8.64 (s, 1H), 8.26-8.15 (m, 2H), 8.07 (t, J=2.0 Hz, 1H), 7.92 (dd, J=8.0, 2.2 Hz, 1H), 7.54 (t, J=8.0 Hz, 1H), 7.25 (d, J=7.7 Hz, 1H), 6.40 (td, J=56.3, 4.6 Hz, 1H), 5.32 (s, 2H), 4.59-4.42 (m, 1H), 4.38-4.30 (m, 1H), 4.30-4.17 (m, 1H), 4.10-3.99 (m, 1H), 3.47 (s, 3H), 3.30-3.09 (m, 1H), 2.00 (d, J=24.3 Hz, 3H); LCMS: C₂₆H₂₁F₈N₅O₂ requires: 587, found: m/z=588 [M+H]⁺.

### Example 440: (R)-6-(5-fluoropyridin-3-yl)-2-(3-(1,1,2-trifluoro-1-(4-methyl-4H-1,2,4-triazol-3-yl)propan-2-yl)phenyl)-4-(trifluoromethyl)isoindolin-1-one (440)

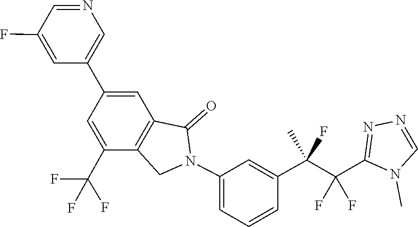 [SMILES: Cn1cnnc1C(F)(F)[C@](C)(F)c1cccc(N2Cc3c(cc(-c4cncc(F)c4)cc3C(F)(F)F)C2=O)c1]

6-Bromo-2-(3-(1,1,2-trifluoro-1-(4-methyl-4H-1,2,4-triazol-3-yl)propan-2-yl)phenyl)-4-(trifluoromethyl)isoindolin-1-one (51 mg, 0.095 mmol, 1.0 eq.), (5-fluoropyridin-3-yl)boronic acid (28 mg, 0.20 mmol, 2.1 eq.) and [1,1′-bis(diphenylphosphino)ferrocene]dichloropalladium(II) dichloromethane adduct (5.0 mg, 0.061 mmol, 6.4 mol %) were added to a reaction vessel followed by dioxane (1 mL). 2M-Potassium carbonate solution (150 μL. 0.30 mmol, 3.2 eq.) was added and the reaction was purged with nitrogen. The reaction was heated in a microwave at 110° C. for 15 minutes. After cooling, water and chloroform: isopropyl alcohol (2:1) were added and the reaction partitioned. The product was extracted with chloroform:isopropyl alcohol (2:1, 2×). The combined organic layers were dried, filtered and concentrated. The crude material was purified by silica gel column chromatography using a gradient of methanol in EtOAc (0-20%) to afford the title compound (44 mg, 85%) as a colorless solid. ¹H NMR (500 MHz, DMSO-d6) δ 8.99 (t, J=1.8 Hz, 1H), 8.67 (d, J=2.7 Hz, 1H), 8.62 (s, 1H), 8.49-8.42 (m, 2H), 8.37 (dt, J=10.3, 2.3 Hz, 1H), 8.04 (d, J=2.0 Hz, 1H), 7.99-7.92 (m, 1H), 7.53 (t, J=8.0 Hz, 1H), 7.23 (d, J=7.9 Hz, 1H), 5.27 (d, J=2.3 Hz, 2H), 3.46 (s, 3H), 2.06-1.94 (m, 3H); LCMS: C₂₆H₁₈F₇N₅O requires: 549, found: m/z=550 [M+H]⁺.

### Example 441: (R)-6-(4-Methylpiperazin-1-yl)-2-(3-(1,1,2-trifluoro-1-(4-methyl-4H-1,2,4-triazol-3-yl)propan-2-yl)phenyl)-4-(trifluoromethyl)isoindolin-1-one (441)

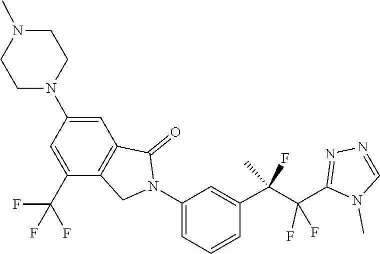 [SMILES: CN1CCN(c2cc3c(c(C(F)(F)F)c2)CN(c2cccc([C@@](C)(F)C(F)(F)c4nncn4C)c2)C3=O)CC1]

The C—N coupling reaction was carried out in a similar fashion as for 386 using (R)-6-bromo-2-(3-(1,1,2-trifluoro-1-(4-methyl-4H-1,2,4-triazol-3-yl)propan-2-yl)phenyl)-4-(trifluoromethyl)isoindolin-1-one (50 mg, 0.094 mmol, 1 eq.) and 1-methylpiperazine (19 mg, 0.19 mmol, 2 eq.) as reactants afforded the title compound (6 mg, 12%) as an off-white solid: ¹H NMR (500 MHz, DMSO-d₆) δ 8.55 (s, 1H), 7.91 (d, J=2.2 Hz, 1H), 7.88-7.81 (m, 1H), 7.50-7.35 (m, 3H), 7.17-7.08 (m, 1H), 5.04-4.87 (m, 2H), 3.37 (s, 3H), 3.30-3.20 (m, 4H), 2.61-2.24 (m, 4H), 2.17 (s, 3H), 1.92 (d, J=24.3 Hz, 3H); LCMS: C₂₆H₂₆F₆N₆O requires: 552, found: m/z=553 [M+H]⁺.

### Example 442: (R)-6-(Piperazin-1-yl)-2-(3-(1,1,2-trifluoro-1-(4-methyl-4H-1,2,4-triazol-3-yl)propan-2-yl)phenyl)-4-(trifluoromethyl)isoindolin-1-one (442)

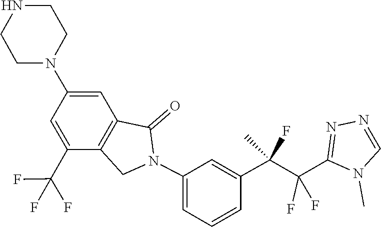 [SMILES: Cn1cnnc1C(F)(F)[C@](C)(F)c1cccc(N2Cc3c(cc(N4CCNCC4)cc3C(F)(F)F)C2=O)c1]

The C—N coupling reaction was carried out in a similar fashion as for 386 using (R)-6-bromo-2-(3-(1,1,2-trifluoro-1-(4-methyl-4H-1,2,4-triazol-3-yl)propan-2-yl)phenyl)-4-(trifluoromethyl)isoindolin-1-one (100 mg, 0.19 mmol, 1 eq.) and piperazine (81 mg, 0.94 mmol, 5 eq.) as reactants afforded the title compound (37 mg, 37%) as an off-white solid: ¹H NMR (500 MHz, DMSO-d₆) δ 8.63 (s, 1H), 8.00 (d, J=2.2 Hz, 1H), 7.92 (ddd, J=8.5, 2.4, 1.0 Hz, 1H), 7.58-7.49 (m, 2H), 7.45 (d, J=2.7 Hz, 1H), 7.21 (d, J=8.6 Hz, 1H), 5.13-5.01 (m, 2H), 3.45 (s, 3H), 3.26-3.16 (m, 4H), 2.97-2.84 (m, 4H), 2.05-1.89 (m, 3H); LCMS: C₂₅H₂₄F₆N₆O requires: 538, found: m/z=539 [M+H]⁺.

### Example 443: 6-((R)-2,4-Dimethylpiperazin-1-yl)-2-(3-((R)-1,1,2-trifluoro-1-(4-methyl-4H-1,2,4-triazol-3-yl)propan-2-yl)phenyl)-4-(trifluoromethyl)isoindolin-1-one (443)

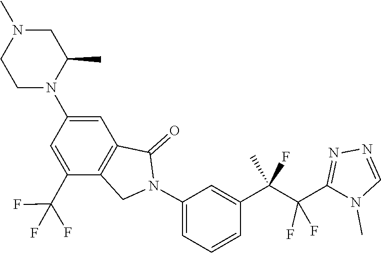 [SMILES: C[C@@H]1CN(C)CCN1c1cc2c(c(C(F)(F)F)c1)CN(c1cccc([C@@](C)(F)C(F)(F)c3nncn3C)c1)C2=O]

The C—N coupling reaction was carried out in a similar fashion as for 386 using (R)-6-bromo-2-(3-(1,1,2-trifluoro-1-(4-methyl-4H-1,2,4-triazol-3-yl)propan-2-yl)phenyl)-4-(trifluoromethyl)isoindolin-1-one (100 mg, 0.19 mmol, 1 eq.) and (3R)-1,3-dimethylpiperazine (26 mg, 0.23 mmol, 1.2 eq.) as reactants afforded the title compound (17 mg, 16%) as an off-white solid: ¹H NMR (500 MHz, DMSO-d₆) δ 8.63 (s, 1H), 7.99 (t, J=1.9 Hz, 1H), 7.91 (dd, J=8.0, 2.2 Hz, 1H), 7.52 (t, J=8.1 Hz, 1H), 7.43 (dd, J=18.8, 2.3 Hz, 2H), 7.21 (d, J=7.8 Hz, 1H), 5.12-4.99 (m, 2H), 4.26 (s, 1H), 3.51 (d, J=12.0 Hz, 1H), 3.44 (s, 3H), 3.09 (td, J=11.8, 3.4 Hz, 1H), 2.87 (d, J=11.2 Hz, 1H), 2.75-2.68 (m, 1H), 2.26 (dd, J=11.2, 3.6 Hz, 1H), 2.23 (s, 3H), 2.10-2.03 (m, 1H), 2.00 (d, J=24.3 Hz, 3H), 1.08 (d, J=6.6 Hz, 3H); LCMS: C₂₇H₂₈F₆N₆O requires: 566, found: m/z=567 [M+H]⁺.

### Example 444: 6-((S)-2,4-Dimethylpiperazin-1-yl)-2-(3-((R)-1,1,2-trifluoro-1-(4-methyl-4H-1,2,4-triazol-3-yl)propan-2-yl)phenyl)-4-(trifluoromethyl)isoindolin-1-one (444)

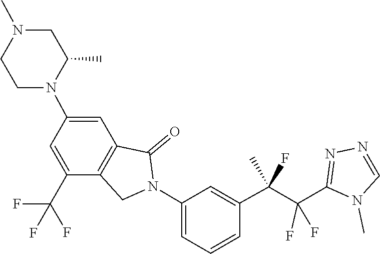 [SMILES: C[C@H]1CN(C)CCN1c1cc2c(c(C(F)(F)F)c1)CN(c1cccc([C@@](C)(F)C(F)(F)c3nncn3C)c1)C2=O]

The C—N coupling reaction was carried out in a similar fashion as for 386 using (R)-6-bromo-2-(3-(1,1,2-trifluoro-1-(4-methyl-4H-1,2,4-triazol-3-yl)propan-2-yl)phenyl)-4-(trifluoromethyl)isoindolin-1-one (100 mg, 0.19 mmol, 1 eq.) and (3S)-1,3-dimethylpiperazine (26 mg, 0.23 mmol, 1.2 eq.) as reactants afforded the title compound (4.3 mg, 4%) as an off-white solid: ¹H NMR (500 MHz, DMSO-d₆) δ 8.55 (s, 1H), 7.90 (s, 1H), 7.84 (dd, J=7.6, 2.1 Hz, 1H), 7.44 (t, J=8.1 Hz, 1H), 7.35 (dd, J=19.4, 2.0 Hz, 2H), 7.16-7.10 (m, 1H), 4.98 (s, 2H), 4.19 (s, 1H), 3.43 (d, J=11.9 Hz, 1H), 3.36 (s, 3H), 3.05-2.96 (m, 1H), 2.80 (d, J=11.1 Hz, 1H), 2.64 (d, J=10.9 Hz, 1H), 2.18 (dd, J=11.1, 3.7 Hz, 1H), 2.15 (s, 3H), 2.02-1.94 (m, 1H), 1.92 (d, J=24.3 Hz, 3H), 1.06-0.96 (m, 3H); LCMS: C₂₇H₂₈F₆N₆O requires: 566, found: m/z=567 [M+H]⁺.

### Example 445: 6-(3-Methyl-3,8-diazabicyclo[3.2.1]octan-8-yl)-2-(3-((R)-1,1,2-trifluoro-1-(4-methyl-4H-1,2,4-triazol-3-yl)propan-2-yl)phenyl)-4-(trifluoromethyl)isoindolin-1-one (445)

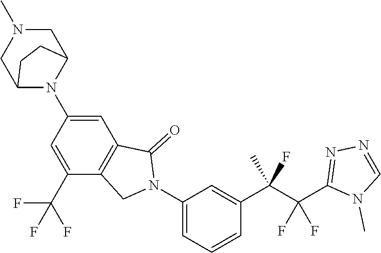 [SMILES: CN1CC2CCC(C1)N2c1cc2c(c(C(F)(F)F)c1)CN(c1cccc([C@@](C)(F)C(F)(F)c3nncn3C)c1)C2=O]

The C—N coupling reaction was carried out in a similar fashion as for 386 using (R)-6-bromo-2-(3-(1,1,2-trifluoro-1-(4-methyl-4H-1,2,4-triazol-3-yl)propan-2-yl)phenyl)-4-(trifluoromethyl)isoindolin-1-one (100 mg, 0.19 mmol, 1 eq.) and 3-methyl-3,8-diazabicyclo[3.2.1]octane dihydrochloride (45 mg, 0.23 mmol, 1.2 eq.) as reactants afforded the title compound (trifluoroacetic acid salt, 1.6 mg, 2%) as an off-white solid: ¹H NMR (500 MHz, DMSO-d₆) δ 9.30 (s, 1H), 8.56 (s, 1H), 7.91 (t, J=2.0 Hz, 1H), 7.85 (dd, J=8.1, 2.2 Hz, 1H), 7.49-7.42 (m, 2H), 7.16 (d, J=8.4 Hz, 1H), 6.46 (s, 1H), 5.01 (s, 2H), 4.69 (d, J=4.0 Hz, 2H), 3.02 (t, J=11.2 Hz, 2H), 2.65 (d, J=4.2 Hz, 3H), 2.50-2.24 (m, 2H), 2.09-1.96 (m, 4H), 1.92 (d, J=24.3 Hz, 3H); LCMS: C₂₈H₂₈F₆N₆O requires: 578, found: m/z=579 [M+H]⁺.

### Example 446: 6-(1-hydroxyethyl)-2-(3-((R)-1,1,2-trifluoro-1-(4-methyl-4H-1,2,4-triazol-3-yl)propan-2-yl)phenyl)-4-(trifluoromethyl)isoindolin-1-one (446)

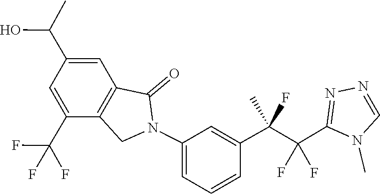 [SMILES: CC(O)c1cc2c(c(C(F)(F)F)c1)CN(c1cccc([C@@](C)(F)C(F)(F)c3nncn3C)c1)C2=O]

Reductive amination of the methyl ketone was carried out according to the procedure for 459, step 2 using (R)-6-(1-ethoxyvinyl)-2-(3-(1,1,2-trifluoro-1-(4-methyl-4H-1,2,4-triazol-3-yl)propan-2-yl)phenyl)-4-(trifluoromethyl)isoindolin-1-one (51 mg, 0.10 mmol, 1.0 eq.) and (S)-3-fluoropyrrolidine hydrochloride (60 mg, 0.48 mmol, 4.9 eq.) as reactants which afforded the title compound (9.5 mg, 20% yield). ¹H NMR (500 MHz, DMSO-d6) δ 8.61 (s, 1H), 8.03 (s, 1H), 8.01-7.97 (m, 2H), 7.91 (dd, J=8.3, 2.1 Hz, 1H), 7.51 (t, J=8.0 Hz, 1H), 7.20 (d, J=7.9 Hz, 1H), 5.26-5.10 (m, 2H), 4.96 (q, J=6.5 Hz, 1H), 1.98 (d, J=24.2 Hz, 3H), 1.39 (d, J=6.5 Hz, 3H); LCMS: C₂₃H₂₀F₆N₄O₂ requires: 498, found: m/z=499 [M+H]⁺.

### Example 447: 6-(((S)-3-fluoropyrrolidin-1-yl)methyl)-2-(3-((R)-1,1,2-trifluoro-1-(4-methyl-4H-1,2,4-triazol-3-yl)propan-2-yl)phenyl)-4-(trifluoromethyl)isoindolin-1-one (447)

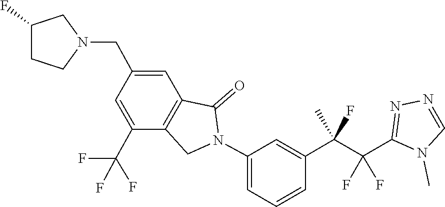 [SMILES: Cn1cnnc1C(F)(F)[C@](C)(F)c1cccc(N2Cc3c(cc(CN4CC[C@H](F)C4)cc3C(F)(F)F)C2=O)c1]

Step 1: Synthesis of (R)-6-bromo-2-(3-(1,1,2-trifluoro-1-(4-methyl-4H-1,2,4-triazol-3-yl)propan-2-yl)phenyl)-4-(trifluoromethyl)isoindolin-1-one. The indolone formation reaction was carried out in a manner similar to 260, step 2 using (R)-3-(1,1,2-trifluoro-1-(4-methyl-4H-1,2,4-triazol-3-yl)propan-2-yl)aniline (741 mg, 1.97 mmol, 1.0 eq.) and methyl 5-bromo-2-(bromomethyl)-3-(trifluoromethyl)benzoate (529 mg, 1.96 mmol, 1.0 eq.) to afford the product in 579 mg (55%) yield.

Step 2: Synthesis of (R)-2-(3-(1,1,2-trifluoro-1-(4-methyl-4H-1,2,4-triazol-3-yl)propan-2-yl)phenyl)-4-(trifluoromethyl)-6-vinylisoindolin-1-one. A mixture of (R)-6-bromo-2-(3-(1,1,2-trifluoro-1-(4-methyl-4H-1,2,4-triazol-3-yl)propan-2-yl)phenyl)-4-(trifluoromethyl)isoindolin-1-one (579 mg, 1.09 mmol, 1.0 eq.), 2,4,6-trivinylcylotriboroxane pyridine complex (318 mg, 1.32 mmol, 1.2 eq.), potassium carbonate (458 mg, 3.31 mmol, 3.0 eq.), SPhos ligand (45 mg, 0.11 mmol, 10 mol %) and dimethoxyethane (2.5 mL) and water (0.25 mL) at rt, was purged with nitrogen. Tris(dibenzylideneacetone)dipalladium(0) (27 mg, 0.029 mmol, 2.6 mol %) was added, purged with nitrogen. The mixture was heated at 80° C. for 2 hours. After cooling to rt, the mixture was diluted with dichloromethane and dried. The mixture was concentrated, and the residue was purified by silica gel column chromatography eluting with EtOAc in hexanes (50-100%) to give the desired product in 456 mg (87%) yield.

Step 3: Synthesis of (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde. (R)-2-(3-(1,1,2-Trifluoro-1-(4-methyl-4H-1,2,4-triazol-3-yl)propan-2-yl)phenyl)-4-(trifluoromethyl)-6-vinylisoindolin-1-one (456 mg, 0.948 mmol, 1 eq.) was dissolved in THF (7.5 mL) and water (2.5 mL) at rt. Potassium osmate(VI) dihydrate (7.5 mg, 0.020 mmol, 2.0 mol %) was added, followed by A-methylmorpholine N-oxide (334 mg, 2.85 mmol, 3.0 eq.). The mixture was stirred at 45° C. overnight. After cooling to rt, solid sodium bisulfite (581 mg, 5.48 mmol, 5.8 eq.) was added and the reaction was diluted with water and dichloromethane. After filtration using Celite, the crude 6-(1,2-dihydroxyethyl)-2-(3-((R)-1,1,2-trifluoro-1-(4-methyl-4H-1,2,4-triazol-3-yl)propan-2-yl)phenyl)-4-(trifluoromethyl)isoindolin-1-one was extracted with dichloromethane three times and the combined dichloromethane layers were dried. Solids were filtered and to the filtrate was added sodium periodate on silica gel (prepared by mixing 11.6 g silica gel, 1.32 g sodium periodate and 5.8 mL water) (5.71 g, 1.90 mmol, 2.0 eq.) and the mixture was stirred vigorously for 15 minutes. After filtration and rinsing the silica gel with dichloromethane:methanol (10:1) (44 mL), all solvents were removed under reduced pressure and the crude material was purified by silica gel column chromatography using a gradient of EtOAc in dichloromethane (50-100%) to afford the desired aldehyde in 377 mg (82%) yield.

Step 4: 6-(((S)-3-fluoropyrrolidin-1-yl)methyl)-2-(3-((R)-1,1,2-trifluoro-1-(4-methyl-4H-1,2,4-triazol-3-yl)propan-2-yl)phenyl)-4-(trifluoromethyl)isoindolin-1-one. (S)-3-fluoropyrrolidine hydrochloride (439 mg, 0.31 mmol, 3.0 eq.), and triethylamine (44 μL, 0.32 mmol, 3.0 eq.) were suspended in dichloromethane (0.5 mL). The mixture was stirred and sonicated to form a uniform suspension. (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (50 mg, 0.10 mmil, 1.0 eq.) was added in dichloromethane (0.3 mL) and the reaction was stirred for 40 minutes. After addition of sodium triacetoxyborohydride (44 mg, 0.21 mmol, 2.0 eq.) the reaction was stirred at rt overnight. After removal of solvent, the residue was purified by reverse phase to afford the title compound as a colorless solid (36 mg, 62% yield). ¹H NMR (500 MHz, DMSO-d6) δ 8.61 (s, 1H), 8.00 (d, J=3.0 Hz, 2H), 7.95 (s, 1H), 7.90 (ddd, J=8.3, 2.3, 0.9 Hz, 1H), 7.51 (t, J=8.1 Hz, 1H), 7.20 (dd, J=7.9, 1.6 Hz, 1H), 5.33-5.10 (m, 3H), 3.84 (s, 2H), 3.43 (s, 3H), 2.91-2.75 (m, 2H), 2.74-2.58 (m, 1H), 2.42-2.32 (m, 1H), 2.17 (ddt, J=21.9, 13.8, 7.0 Hz, 1H), 1.98 (d, J=24.4 Hz, 3H), 1.88 (ddd, J=21.5, 15.4, 7.2 Hz, 1H); LCMS: C₂₆H₂₄F₇N₅O requires: 555, found: m/z=556 [M+H]⁺.

### Example 448: (R)-6-((3,3-difluoropyrrolidin-1-yl)methyl)-2-(3-(1,1,2-trifluoro-1-(4-methyl-4H-1,2,4-triazol-3-yl)propan-2-yl)phenyl)-4-(trifluoromethyl)isoindolin-1-one (448)

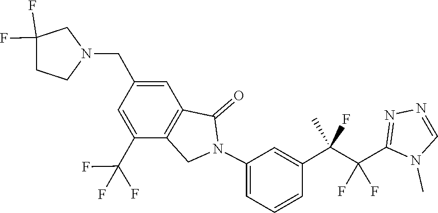 [SMILES: Cn1cnnc1C(F)(F)[C@](C)(F)c1cccc(N2Cc3c(cc(CN4CCC(F)(F)C4)cc3C(F)(F)F)C2=O)c1]

The reductive amination was carried out in a similar fashion as for 411, step 2 using (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (201 mg, 0.416 mmol, 1.0 eq.) and 3,3-difluoropyrrolidine hydrochloride (299 mg, 2.08 mmol, 5.0 eq.) as reactants to afford the title compound (185 mg, 77% yield) as a colorless solid. ¹H NMR (500 MHz, DMSO-d6) δ 8.63 (s, 1H), 8.02 (d, J=3.5 Hz, 2H), 1.91 (s, 1H), 7.92 (ddd, J=8.2, 2.3, 0.9 Hz, 1H), 7.53 (t, J=8.0 Hz, 1H), 7.22 (d, J=7.9 Hz, 1H), 5.20 (d, J=2.7 Hz, 2H), 3.87 (s, 2H), 3.45 (s, 3H), 2.93 (t, J=13.3 Hz, 2H), 2.76 (t, J=7.0 Hz, 2H), 2.30 (tt, J=14.9, 6.9 Hz, 2H), 2.00 (d, J=24.3 Hz, 3H); LCMS: C₂₆H₂₃F₈N₅O requires: 573, found: m/z=574 [M+H]⁺.

### Example 449: 6-(((S)-3-fluoropyrrolidin-1-yl)methyl)-2-(3-((S)-1,1,2-trifluoro-1-(4-methyl-4H-1,2,4-triazol-3-yl)propan-2-yl)phenyl)-4-(trifluoromethyl)isoindolin-1-one (449)

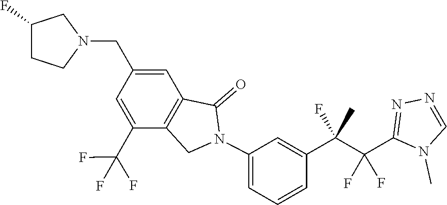 [SMILES: Cn1cnnc1C(F)(F)[C@@](C)(F)c1cccc(N2Cc3c(cc(CN4CC[C@H](F)C4)cc3C(F)(F)F)C2=O)c1]

Step 1: Synthesis of (S)-6-bromo-2-(3-(1,1,2-trifluoro-1-(4-methyl-4H-1,2,4-triazol-3-yl)propan-2-yl)phenyl)-4-(trifluoromethyl)isoindolin-1-one. The indolone formation reaction was carried out in a manner similar to 260, step 2 using (S)-3-(1,1,2-trifluoro-1-(4-methyl-4H-1,2,4-triazol-3-yl)propan-2-yl)aniline (537 mg, 1.99 mmol, 1.0 eq.) and methyl 5-bromo-2-(bromomethyl)-3-(trifluoromethyl)benzoate (753 mg, 2.00 mmol, 1.0 eq.) as reactants to afford the title compound in 634 mg (60%) yield.

Step 2: Synthesis of (S)-2-(3-(1,1,2-trifluoro-1-(4-methyl-4H-1,2,4-triazol-3-yl)propan-2-yl)phenyl)-4-(trifluoromethyl)-6-vinylisoindolin-1-one. The vinyl group was installed according to the procedure for 447, step 2 using (S)-6-bromo-2-(3-(1,1,2-trifluoro-1-(4-methyl-4H-1,2,4-triazol-3-yl)propan-2-yl)phenyl)-4-(trifluoromethyl)isoindolin-1-one (634 mg, 1.19 mmol, 1.0 eq.) as the reactant to afford the title compound in 493 mg (86%) yield.

Step 3: Synthesis of (S)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde. The vinyl group was converted to the aldehyde according to the procedure described for 447, step 3 using (S)-2-(3-(1,1,2-trifluoro-1-(4-methyl-4H-1,2,4-triazol-3-yl)propan-2-yl)phenyl)-4-(trifluoromethyl)-6-vinylisoindolin-1-one (493 mg, 1.03 mmol, 1.0 eq.) as the reactant to afford the title compound in 401 mg (81%) yield.

Step 4: Synthesis of 6-(((S)-3-fluoropyrrolidin-1-yl)methyl)-2-(3-((S)-1,1,2-trifluoro-1-(4-methyl-4H-1,2,4-triazol-3-yl)propan-2-yl)phenyl)-4-(trifluoromethyl)isoindolin-1-one. The reductive amination was carried out in a similar fashion as for 447, step 4 using of (S)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (50 mg, 0.10 mmol, 1.0 eq.) and (S)-3-fluoropyrrolidine hydrochloride (40 mg, 0.32 mmol, 3.1 eq.) as reactants to afford the title compound (42 mg, 72% yield) as a colorless solid. ¹H NMR (500 MHz, DMSO-d6) δ 8.61 (s, 1H), 8.00 (d, J=3.6 Hz, 2H), 7.95 (s, 1H), 7.90 (ddd, J=8.2, 2.3, 0.9 Hz, 1H), 7.51 (t, J=8.0 Hz, 1H), 7.20 (d, J=8.1 Hz, 1H), 5.33-5.10 (m, 3H), 3.84 (s, 2H), 3.43 (s, 3H), 2.89-2.76 (m, 2H), 2.74-2.61 (m, 1H), 2.40-2.33 (m, 1H), 2.17 (ddt, J=27.8, 13.8, 6.9 Hz, 1H), 1.98 (d, J=24.3 Hz, 3H), 1.88 (ddd, J=21.2, 15.0, 7.0 Hz, 1H); LCMS: C₂₆H₂₄F₇N₅O requires: 555, found: m/z=556 [M+H]⁺.

### Example 450: 6-(((R)-3-fluoropyrrolidin-1-yl)methyl)-2-(3-((R)-1,1,2-trifluoro-1-(4-methyl-4H-1,2,4-triazol-3-yl)propan-2-yl(phenyl)-4-(trifluoromethyl)isoindolin-1-one (450)

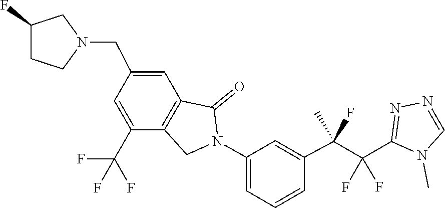 [SMILES: Cn1cnnc1C(F)(F)[C@](C)(F)c1cccc(N2Cc3c(cc(CN4CC[C@@H](F)C4)cc3C(F)(F)F)C2=O)c1]

The reductive amination was carried out in a similar fashion as for 447, step 4 using (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (49 mg, 0.10 mmol, 1.0 eq.) and (R)-3-fluoropyrrolidine hydrochloride (64 mg, 0.51 mmol, 5.0 eq.) as reactants to afford the title compound (39 mg, 68% yield) as a colorless solid. ¹H NMR (500 MHz, DMSO-d6) δ 8.61 (s, 1H), 8.00 (q, J=1.7 Hz, 2H), 7.95 (s, 1H), 7.90 (dd, J=7.9, 2.2 Hz, 1H), 7.51 (t, J=8.1 Hz, 1H), 7.21 (dd, J=7.7, 1.5 Hz, 1H), 5.34-5.10 (m, 3H), 3.84 (s, 2H), 3.43 (s, 3H), 2.89-2.76 (m, 2H), 2.73-2.59 (m, 1H), 2.42-2.32 (m, 1H), 2.17 (ddq, J=28.0, 14.5, 7.0 Hz, 1H), 1.98 (d, J=24.3 Hz, 3H), 1.94-1.81 (m, 1H); LCMS: C₂₆H₂₄F₇N₅O requires: 555, found: m/z=556 [M+H]⁺.

### Example 451: (R)-6-((3-fluoroazetidin-1-yl)methyl)-2-(3-(1,1,2-trifluoro-1-(4-methyl-4H-1,2,4-triazol-3-yl)propan-2-yl)phenyl)-4-(trifluoromethyl)isoindolin-1-one (451)

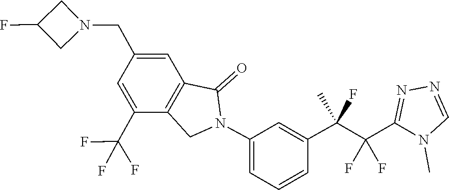 [SMILES: Cn1cnnc1C(F)(F)[C@](C)(F)c1cccc(N2Cc3c(cc(CN4CC(F)C4)cc3C(F)(F)F)C2=O)c1]

The reductive amination was carried out in a similar fashion as for 447, step 4 using of (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (50 mg, 0.10 mmol, 1.0 eq.) and 3-fluoroazetidine hydrochloride (60 mg, 0.54 mmol, 5.1 eq.) as reactants to afford the title compound (11 mg, 19% yield) as a colorless solid. ¹H NMR (500 MHz, DMSO-d6) δ 8.62 (s, 1H), 8.01 (d, J=2.1 Hz, 1H), 7.98 (s, 1H), 7.94-7.88 (m, 2H), 7.52 (t, J=8.0 Hz, 1H), 7.24-7.18 (m, 1H), 5.33-5.10 (m, 3H), 3.86 (s, 2H), 3.68-3.54 (m, 2H), 3.44 (s, 3H), 3.26-3.14 (m, 1H), 1.99 (d, J=24.3 Hz, 3H); LCMS: C₂₅H₂₂F₇N₅O requires: 541, found: m/z=542 [M+H]⁺.

### Example 452: (R)-6-((3-methoxyazetidin-1-yl)methyl)-2-(3-(1,1,2-trifluoro-1-(4-methyl-4H-1,2,4-triazol-3-yl)propan-2-yl)phenyl)-4-(trifluoromethyl)isoindolin-1-one (452)

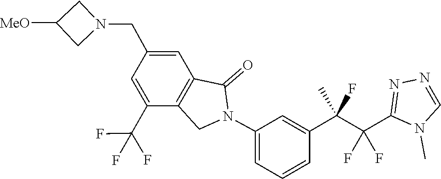 [SMILES: COC1CN(Cc2cc3c(c(C(F)(F)F)c2)CN(c2cccc([C@@](C)(F)C(F)(F)c4nncn4C)c2)C3=O)C1]

The reductive amination was carried out in a similar fashion as for 447, step 4 using of (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (50 mg, 0.10 mmol, 1.0 eq.) and 3-methoxyazetidine hydrochloride (105 mg, 0.85 mmol, 8.2 eq.) as reactants to afford the title compound (30 mg, 53% yield) as a colorless solid. ¹H NMR (500 MHz, DMSO-d6) δ 8.62 (s, 1H), 8.01 (d, J=2.0 Hz, 1H), 7.96 (s, 1H), 7.93-7.87 (m, 2H), 7.52 (t, J=8.0 Hz, 1H), 7.21 (d, J=7.8 Hz, 1H), 5.18 (s, 2H), 4.02 (p, J=5.7 Hz, 1H), 3.80 (s, 2H), 3.51 (td, J=6.1, 1.9 Hz, 2H), 3.44 (s, 3H), 3.16 (s, 3H), 2.97-2.90 (m, 2H), 1.99 (d, J=24.2 Hz, 3H); LCMS: C₂₆H₂₅F₆N₅O₂ requires: 553, found: m/z=554 [M+H]⁺.

### Example 453: (R)-6-((3-(difluoromethyl)azetidin-1-yl)methyl)-2-(3-(1,1,2-trifluoro-1-(4-methyl-4H-1,2,4-triazol-3-yl)propan-2-yl)phenyl)-4-(trifluoromethyl)isoindolin-1-one (453)

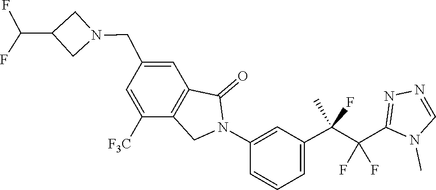 [SMILES: Cn1cnnc1C(F)(F)[C@](C)(F)c1cccc(N2Cc3c(cc(CN4CC(C(F)F)C4)cc3C(F)(F)F)C2=O)c1]

The reductive amination was carried out in a similar fashion as for 447, step 4 using (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (49 mg, 0.1 mmol, 1 eq) and 3-(difluoromethyl)azetidine hydrochloride (73 mg, 0.5 mmol, 5 eq) to afford the title compound (38 mg) as an off-white solid: ¹H NMR (500 MHz, DMSO-d₆) δ 10.57-10.33 (m, 1H), 8.63 (s, 1H), 8.35-8.15 (m, 2H), 8.02 (d, J=2.0 Hz, 1H), 7.93 (dd, J=7.9, 2.2 Hz, 1H), 7.53 (t, J=8.0 Hz, 1H), 7.24 (d, J=7.9 Hz, 1H), 6.34 (t, J=55.9 Hz, 1H), 5.26 (s, 2H), 4.76-4.48 (m, 2H), 4.43-4.06 (m, 4H), 3.48 (s, 3H), 3.39 (s, 1H), 1.99 (d, J=24.3 Hz, 3H); LCMS: C₂₆H₂₃F₈N₅O requires: 573, found: m/z=574 [M+H]⁺.

### Example 454: (S)-1-((3-oxo-2-(3-((R)-1,1,2-trifluoro-1-(4-methyl-4H-1,2,4-triazol-3-yl)propan-2-yl)phenyl)-7-(trifluoromethyl)isoindolin-5-yl)methyl)pyrrolidine-3-carbonitrile (454)

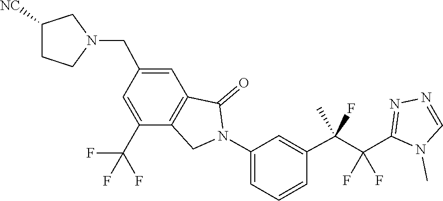 [SMILES: Cn1cnnc1C(F)(F)[C@](C)(F)c1cccc(N2Cc3c(cc(CN4CC[C@H](C#N)C4)cc3C(F)(F)F)C2=O)c1]

The reductive amination was carried out in a similar fashion as for 447, step 4 using of (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (27 mg, 0.057 mmol, 1.0 eq.) and (S)-pyrrolidine-3-carbonitrile hydrochloride (39 mg, 0.29 mmol, 5.2 eq.) as reactants to afford the title compound (18 mg, 57% yield) as a colorless solid. ¹H NMR (500 MHz, DMSO-d6) δ 8.61 (s, 1H), 8.00 (d, J=3.7 Hz, 2H), 7.95 (s, 1H), 7.93-7.87 (m, 1H), 7.51 (t, J=8.0 Hz, 1H), 7.21 (d, J=7.6 Hz, 1H), 5.18 (d, J=2.4 Hz, 2H), 3.85 (s, 2H), 3.44 (s, 3H), 2.85-2.67 (m, 3H), 2.21 (dddd, J=13.2, 9.9, 8.1, 5.5 Hz, 1H), 2.06-1.89 (m, 4H); LCMS: C₂₇H₂₄F₆N₆O requires: 562, found: m/z=563 [M+H]⁺.

### Example 455: (R)-1-((3-oxo-2-(3-((R)-1,1,2-trifluoro-1-(4-methyl-4H-1,2,4-triazol-3-yl)propan-2-yl)phenyl)-7-(trifluoromethyl)isoindolin-5-yl)methyl)pyrrolidine-3-carbonitrile (455)

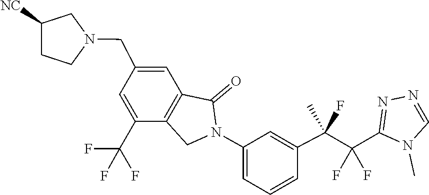 [SMILES: Cn1cnnc1C(F)(F)[C@](C)(F)c1cccc(N2Cc3c(cc(CN4CC[C@@H](C#N)C4)cc3C(F)(F)F)C2=O)c1]

The reductive amination was carried out in a similar fashion as for 447, step 4 using of (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (31 mg, 0.064 mmol, 1.0 eq.) and (R)-pyrrolidine-3-carbonitrile hydrochloride (42 mg, 0.32 mmol, 5.0 eq.) as reactants to afford the title compound (30 mg, 83% yield) as a colorless solid. ¹H NMR (500 MHz, DMSO-d6) δ 8.61 (s, 1H), 8.00 (d, J=3.0 Hz, 2H), 7.95 (s, 1H), 7.93-7.88 (m, 1H), 7.51 (t, J=8.1 Hz, 1H), 7.21 (d, J=8.1 Hz, 1H), 5.18 (s, 2H), 3.85 (s, 2H), 3.44 (s, 3H), 2.83-2.65 (m, 3H), 2.27-2.15 (m, 1H), 2.04-1.91 (m, 4H); LCMS: C₂₇H₂₄F₆N₆O requires: 562, found: m/z=563 [M+H]⁺.

### Example 456: (R)-1-((3-oxo-2-(3-(1,1,2-trifluoro-1-(4-methyl-4H-1,2,4-triazol-3-yl)propan-2-yl)phenyl)-7-(trifluoromethyl)isoindolin-5-yl)methyl)azetidine-3-carbonitrile (456)

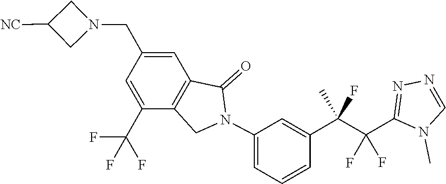 [SMILES: Cn1cnnc1C(F)(F)[C@](C)(F)c1cccc(N2Cc3c(cc(CN4CC(C#N)C4)cc3C(F)(F)F)C2=O)c1]

The reductive amination was carried out in a similar fashion as for 411, step 2 using (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (31 mg, 0.064 mmol, 1.0 eq.) and azetidine-3-carbonitrile hydrochloride (73 mg, 0.62 mmol, 9.6 eq.) as reactants to afford the title compound (13 mg, 37% yield) as a colorless solid. ¹H NMR (500 MHz, DMSO-d6) δ 8.61 (s, 1H), 8.00 (s, 1H), 7.96 (s, 1H), 7.93-7.87 (m, 2H), 7.51 (t, J=8.0 Hz, 1H), 7.20 (d, J=8.0 Hz, 1H), 5.17 (s, 2H), 3.80 (s, 2H), 3.59-3.45 (m, 2H), 3.44 (s, 3H), 3.34 (dd, J=6.5, 5.0 Hz, 2H), 1.98 (d, J=24.2 Hz, 3H); LCMS: C₂₆H₂₂F₆N₆O requires: 548, found: m/z=549 [M+H]⁺.

### Example 457: 6-(((S)-3-(Methylsulfonyl)pyrrolidin-1-yl)methyl)-2-(3-((R)-1,1,2-trifluoro-1-(4-methyl-4H-1,2,4-triazol-3-yl)propan-2-yl)phenyl)-4-(trifluoromethyl)isoindolin-1-one (457)

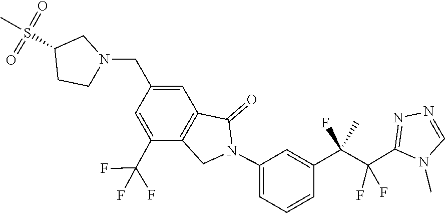 [SMILES: Cn1cnnc1C(F)(F)[C@](C)(F)c1cccc(N2Cc3c(cc(CN4CC[C@H](S(C)(=O)=O)C4)cc3C(F)(F)F)C2=O)c1]

The reductive amination was carried out in a similar fashion as for 447, step 4 using (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (50 mg, 0.10 mmol, 1 eq.) and (3 S)-3-methanesulfonylpyrrolidine (77 mg, 0.52 mmol, 5 eq.) as reactants afforded the title compound (16 mg, 24%) as an off-white solid: ¹H NMR (500 MHz, DMSO-d₆) δ 8.64 (s, 1H), 8.34 (s, 1H), 8.26 (s, 1H), 8.04 (d, J=2.0 Hz, 1H), 7.95 (dd, J=8.3, 2.2 Hz, 1H), 7.55 (t, J=8.0 Hz, 1H), 7.26 (d, J=8.0 Hz, 1H), 5.28 (s, 2H), 4.64 (s, 2H), 3.13 (s, 3H), 2.60-2.20 (m, 10H), 2.01 (d, J=24.3 Hz, 3H); LCMS: C₂₇H₂₇F₆N₅O₃S requires: 615, found: m/z=616 [M+H]⁺.

### Example 458: 6-(((R)-3-(Methylsulfonyl)pyrrolidin-1-yl)methyl)-2-(3-((R)-1,1,2-trifluoro-1-(4-methyl-4H-1,2,4-triazol-3-yl)propan-2-yl)phenyl)-4-(trifluoromethyl)isoindolin-1-one (458)

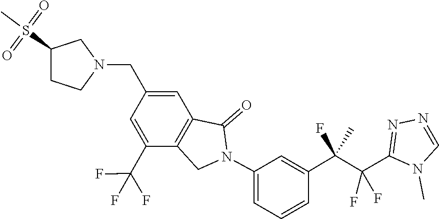 [SMILES: Cn1cnnc1C(F)(F)[C@](C)(F)c1cccc(N2Cc3c(cc(CN4CC[C@@H](S(C)(=O)=O)C4)cc3C(F)(F)F)C2=O)c1]

The reductive amination was carried out in a similar fashion as for 447, step 4 using (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (50 mg, 0.10 mmol, 1 eq.) and (3R)-3-methanesulfonylpyrrolidine (77 mg, 0.52 mmol, 5 eq.) as reactants afforded the title compound (16 mg, 24%) as an off-white solid: ¹H NMR (500 MHz, DMSO-d₆) δ 8.64 (s, 1H), 8.34 (s, 1H), 8.27 (s, 1H), 8.03 (t, J=1.9 Hz, 1H), 7.95 (dd, J=8.0, 2.1 Hz, 1H), 7.55 (t, J=8.1 Hz, 1H), 7.26 (d, J=7.7 Hz, 1H), 5.28 (s, 2H), 4.65 (s, 2H), 3.13 (s, 3H), 2.60-2.20 (m, 10H), 2.01 (d, J=24.3 Hz, 3H); LCMS: C₂₇H₂₇F₆N₅O₃S requires: 615, found: m/z=616 [M+H]⁺.

### Examples 459 and 460: 6-((R)-1-((S)-3-fluoropyrrolidin-1-yl)ethyl)-2-(3-((R)-1,1,2-trifluoro-1-(4-methyl-4H-1,2,4-triazol-3-yl)propan-2-yl)phenyl)-4-(trifluoromethyl)isoindolin-1-one (459) and 6-((S)-1-((S)-3-fluoropyrrolidin-1-yl)ethyl)-2-(3-((R)-1,1,2-trifluoro-1-(4-methyl-4H-1,2,4-triazol-3-yl)propan-2-yl)phenyl)-4-(trifluoromethyl)isoindolin-1-one (460)

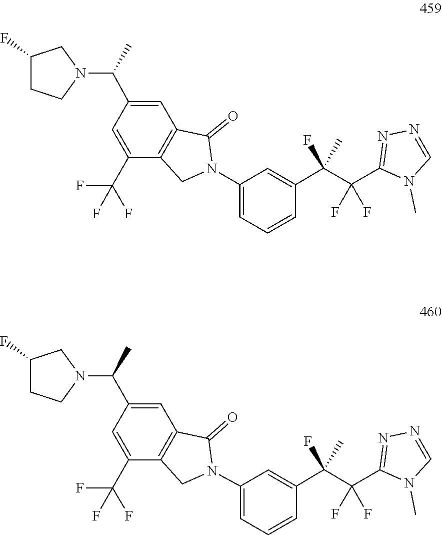 [SMILES: C[C@H](c1cc2c(c(C(F)(F)F)c1)CN(c1cccc([C@@](C)(F)C(F)(F)c3nncn3C)c1)C2=O)N1CC[C@H](F)C1 ; C[C@@H](c1cc2c(c(C(F)(F)F)c1)CN(c1cccc([C@@](C)(F)C(F)(F)c3nncn3C)c1)C2=O)N1CC[C@H](F)C1]

Step 1: Synthesis of (R)-6-acetyl-2-(3-(1,1,2-trifluoro-1-(4-methyl-4H-1,2,4-triazol-3-yl)propan-2-yl)phenyl)-4-(trifluoromethyl)isoindolin-1-one. (R)-6-Bromo-2-(3-(1,1,2-trifluoro-1-(4-methyl-4H-1,2,4-triazol-3-yl)propan-2-yl)phenyl)-4-(trifluoromethyl)isoindolin-1-one (314 mg, 0.589 mmol, 1.0 eq.) was dissolved in toluene (2 mL) at rt. Tributyl(1-ethoxyvinyl)stannane (236 μL, 0.706 mmol, 1.2 eq.) was added, followed by the addition of bis(triphenylphosphine)palladium(II) dichloride (41 mg, 0.058 mmol, 10 mol %). After purging with nitrogen, the reaction was heated at 100° C. overnight. The cooled reaction mixture was loaded directly onto a silica gel column and purified using a gradient of EtOAc in hexanes (50-100%) to yield (R)-6-(1-ethoxyvinyl)-2-(3-(1,1,2-trifluoro-1-(4-methyl-4H-1,2,4-triazol-3-yl)propan-2-yl)phenyl)-4-(trifluoromethyl)isoindolin-1-one (276 mg, 0.525 mmol, 89% yield). This vinyl ether product was stirred under a heterogeneous mixture of dichloromethane and 1N-hydrochloric acid for several hours at rt to hydrolyze to the ketone product. After separation of layers, dichloromethane extraction of the aqueous layer (×2), drying of the combined organic layer with anhydrous sodium sulfate, filtration and removal of solvent under reduced pressure afforded the title compound (216 mg, 0.436 mmol).

Step 2: Synthesis of 6-(1-((S)-3-fluoropyrrolidin-1-yl)ethyl)-2-(3-((R)-1,1,2-trifluoro-1-(4-methyl-4H-1,2,4-triazol-3-yl)propan-2-yl)phenyl)-4-(trifluoromethyl)isoindolin-1-one. (R)-6-acetyl-2-(3-(1,1,2-trifluoro-1-(4-methyl-4H-1,2,4-triazol-3-yl)propan-2-yl)phenyl)-4-(trifluoromethyl)isoindolin-1-one (51 mg, 0.10 mmol, 1.0 eq.), (S)-3-fluoropyrrolidine hydrochloride (66 mg, 0.52 mmol, 5.1 eq.) and triethylamine (70 μL, 0.50 mmol, 4.9 eq.) were dissolved in methanol (0.5 mL). The mixture was heated in a microwave for 1 minute at 100° C. Sodium cyanoborohydride (12 mg, 0.19 mmol, 1.8 eq.) was added and the reaction was microwaved at 80° C. for 30 minutes. The reaction was quenched with a few drops of 1N-hydrochloric acid. After the bubbling had ceased, the reaction was basified with sat. sodium bicarbonate and the product was extracted using chloroform:isopropyl alcohol (2:1) mixture (×3). The combined organic layers were dried. The crude material obtained after removal of solvent under reduced pressure was purified by reverse phase HPLC using a gradient of acetonitrile in water with 0.1% trifluoroacetic acid. The desired product was obtained (34 mg, 57%) as a colorless solid.

Step 3: Synthesis of 6-((R)-1-((S)-3-fluoropyrrolidin-1-yl)ethyl)-2-(3-((S)-1,1,2-trifluoro-1-(4-methyl-4H-1,2,4-triazol-3-yl)propan-2-yl)phenyl)-4-(trifluoromethyl)isoindolin-1-one and 6-((S)-1-((S)-3-fluoropyrrolidin-1-yl)ethyl)-2-(3-((S)-1,1,2-trifluoro-1-(4-methyl-4H-1,2,4-triazol-3-yl)propan-2-yl)phenyl)-4-(trifluoromethyl)isoindolin-1-one. The diastereomers of 6-(1-((S)-3-Fluoropyrrolidin-1-yl)ethyl)-2-(3-((R)-1,1,2-trifluoro-1-(4-methyl-4H-1,2,4-triazol-3-yl)propan-2-yl)phenyl)-4-(trifluoromethyl)isoindolin-1-one (33 mg) were separated using IA column with CO₂ and methanol as mobile phase to afford:

6-((R)-1-((S)-3-fluoropyrrolidin-1-yl)ethyl)-2-(3-((S)-1,1,2-trifluoro-1-(4-methyl-4H-1,2,4-triazol-3-yl)propan-2-yl)phenyl)-4-(trifluoromethyl)isoindolin-1-one (459): (13 mg) ¹H NMR (500 MHz, DMSO-d6) δ 8.61 (s, 1H), 8.02 (s, 1H), 7.98 (t, J=1.9 Hz, 1H), 7.95 (s, 1H), 7.90 (ddd, J=8.1, 2.2, 0.9 Hz, 1H), 7.51 (t, J=8.0 Hz, 1H), 7.21 (d, J=7.8 Hz, 1H), 5.31-5.07 (m, 3H), 3.63 (q, J=6.6 Hz, 1H), 3.43 (s, 3H), 2.88 (dd, J=26.9, 11.5 Hz, 1H), 2.69-2.54 (m, 2H), 2.43-2.32 (m, 1H), 2.11 (ddt, J=28.6, 14.0, 6.9 Hz, 1H), 1.98 (d, J=24.2 Hz, 3H), 1.88 (ddd, J=31.1, 14.4, 7.2 Hz, 1H), 1.36 (d, J=6.5 Hz, 3H); LCMS: C₂₇H₂₆F₇N₅O requires: 569, found: m/z=570 [M+H]⁺.

6-((S)-1-((S)-3-fluoropyrrolidin-1-yl)ethyl)-2-(3-((S)-1,1,2-trifluoro-1-(4-methyl-4H-1,2,4-triazol-3-yl)propan-2-yl)phenyl)-4-(trifluoromethyl)isoindolin-1-one (460): (16 mg) ¹H NMR (500 MHz, DMSO-d6) δ 8.61 (s, 1H), 8.02 (s, 1H), 7.99 (d, J=2.1 Hz, 1H), 7.96 (s, 1H), 7.90 (dt, J=8.0, 1.4 Hz, 1H), 7.51 (t, J=8.0 Hz, 1H), 7.21 (d, J=8.1 Hz, 1H), 5.32-5.07 (m, 3H), 3.60 (q, J=6.5 Hz, 1H), 3.43 (s, 3H), 2.92 (td, J=8.2, 5.1 Hz, 1H), 2.71-2.55 (m, 2H), 2.32-2.08 (m, 2H), 1.98 (d, J=24.3 Hz, 3H), 1.93-1.82 (m, 1H), 1.36 (d, J=6.5 Hz, 3H); LCMS: C₂₇H₂₆F₇N₅O requires: 569, found: m/z=570 [M+H]⁺.

### Example 461: 6-(((3R,7S)-3,4-difluoropyrrolidin-1-yl)methyl)-2-(3-((R)-1,1,2-trifluoro-1-(4-methyl-4H-1,2,4-triazol-3-yl)propan-2-yl)phenyl)-4-(trifluoromethyl)isoindolin-1-one (461)

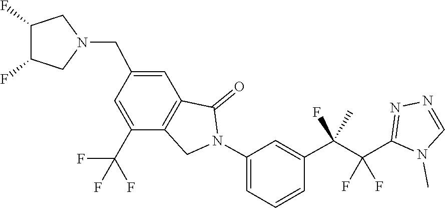 [SMILES: Cn1cnnc1C(F)(F)[C@](C)(F)c1cccc(N2Cc3c(cc(CN4C[C@@H](F)[C@@H](F)C4)cc3C(F)(F)F)C2=O)c1]

The reductive amination was carried out in a similar fashion as for 447, step 4 using (R)-3-oxo-2-(3-(1,1,2-trifluoro-1-(4-methyl-4H-1,2,4-triazol-3-yl)propan-2-yl)phenyl)-7-(trifluoromethyl)isoindoline-5-carbaldehyde (41 mg, 0.085 mmol, 1.0 eq.) and cis-3,4-difluoropyrrolidine hydrochloride (55 mg, 0.38 mmol, 4.5 eq.) as reactants to afford the title compound (30 mg, 62% yield) as a colorless solid. ¹H NMR (500 MHz, DMSO-d₆) δ 8.61 (s, 1H), 8.00 (t, J=2.4 Hz, 2H), 7.95 (s, 1H), 7.93-7.87 (m, 1H), 7.51 (t, J=8.0 Hz, 1H), 7.21 (d, J=7.9 Hz, 1H), 5.30-5.06 (m, 4H), 3.88 (s, 2H), 3.44 (s, 3H), 2.97-2.79 (m, 4H), 1.98 (d, J=24.2 Hz, 3H); LCMS: C₂₆H₂₃F₈N₅O requires: 573, found: m/z=574 [M+H]⁺.

### Example 462: (S)-2-(3-(1-(difluoro(4-methyl-4H-1,2,4-triazol-3-yl)methyl)cyclopropyl)phenyl)-6-((3-fluoropyrrolidin-1-yl)methyl)-4-(trifluoromethyl)isoindolin-1-one (462)

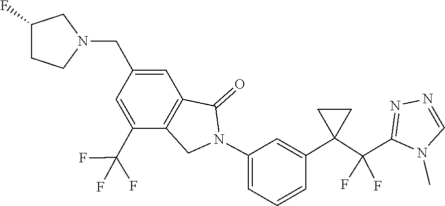 [SMILES: Cn1cnnc1C(F)(F)C1(c2cccc(N3Cc4c(cc(CN5CC[C@H](F)C5)cc4C(F)(F)F)C3=O)c2)CC1]

Step 1: Synthesis of 2-(3-(l-(difluoro(4-methyl-4H-1,2,4-triazol-3-yl)methyl)cyclopropyl)phenyl)-3-oxo-7-(trifluoromethyl)isoindoline-5-carbaldehyde. The indolone formation reaction was carried out in a manner similar to 260, step 2 using 3-(1-(difluoro(4-methyl-4H-1,2,4-triazol-3-yl)methyl)cyclopropyl)aniline (53 mg, 0.20 mmol, 1.0 eq.) and methyl 2-(bromomethyl)-5-formyl-3-(trifluoromethyl)benzoate (65 mg, 0.20 mmol, 1.0 eq.) as reactants to afford the title compound in 20 mg (21%) yield.

Step 2: Synthesis of (S)-2-(3-(l-(difluoro(4-methyl-4H-1,2,4-triazol-3-yl)methyl)cyclopropyl)phenyl)-6-((3-fluoropyrrolidin-1-yl)methyl)-4-(trifluoromethyl)isoindolin-1-one. The reductive animation was carried out in a similar fashion as for 447, step 4 using 2-(3-(1-(difluoro(4-methyl-7H-1,2,4-triazol-3-yl)methyl)cyclopropyl)phenyl)-3-oxo-7-(trifluoromethyl)isoindoline-5-carbaldehyde (20 mg, 0.041 mmol, 1.0 eq.) and (S)-3-fluoropyrrolidine hydrochloride (24 mg, 0.19 mmol, 4.6 eq.) as reactants to afford the title compound (14 mg, 59% yield) as a colorless solid, ¹H NMR (500 MHz, DMSO-d6) δ 8.49 (s, 1H), 7.99 (s, 1H), 7.94 (s, 1H), 7.87 (t, J=2.0 Hz, 1H), 7.83 (ddd, J=8.2, 2.3, 1.0 Hz, 1H), 7.36 (t, J=7.9 Hz, 1H), 7.18-7.12 (m, 1H), 5.21 (dt, J=55.7, 6.0 Hz, 1H), 5.11 (s, 2H), 3.83 (s, 2H), 3.37 (d, J=1.2 Hz, 3H), 2.88-2.74 (m, 2H), 2.74-2.59 (m, 1H), 2.42-2.33 (m, 1H), 2.16 (ddq, J=27.9, 14.1, 6.8 Hz, 1H), 1.90 (ddt, J=29.4, 14.3, 7.0 Hz, 1H), 1.48-1.38 (m, 2H), 1.14 (s, 2H); LCMS: C₂₇H₂₅F₆N₅O requires: 549, found: m/z=550 [M+H]⁺.

### Example 463: 6-((S)-3-hydroxypyrrolidine-1-carbonyl)-2-(3-((R)-1-(4-methyl-1H-pyrazol-3-yl)propan-2-yl)phenyl)-4-(trifluoromethyl)isoindolin-1-one (463)

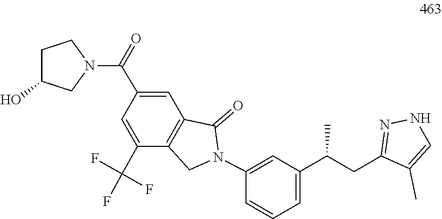 [SMILES: Cc1c[nH]nc1C[C@@H](C)c1cccc(N2Cc3c(cc(C(=O)N4CC[C@@H](O)C4)cc3C(F)(F)F)C2=O)c1]

Step 1: Synthesis of (R)-2-(3-(1-(4-methyl-1H-pyrazol-3-yl)propan-2-yl)phenyl)-3-oxo-7-(trifluoromethyl)isoindoline-5-carboxylic acid. The indolone formation reaction was carried out in a manner similar to 260, step 2 using 4-(bromomethyl)-3-(methoxycarbonyl)-5-(trifluoromethyl)benzoic acid (350 mg, 1.03 mmol) in methanol (5 mL) and (R)-3-(1-(4-methyl-1H-pyrazol-3-yl)propan-2-yl)aniline (220 mg, 1.03 mmol) to afford the title compound (250 mg, 55%) as a yellow oil. MS (ESI) calculated for (C₂₃H₂₀F₃N₃O₃) [M+H]⁺, 444.1; found, 444.2.

Step 2: Synthesis of 6-((S)-3-hydroxypyrrolidine-1-carbonyl)-2-(3-((R)-1-(4-methyl-1H-pyrazol-3-yl)propan-2-yl)phenyl)-4-(trifluoromethyl)isoindolin-1-one (463). (R)-2-(3-(1-(4-methyl-1H-pyrazol-3-yl)propan-2-yl)phenyl)-3-oxo-7-(trifluoromethyl)isoindoline-5-carboxylic acid (100 mg, 0.23 mmol) and (S)-pyrrolidin-3-ol hydrochloride (42.0 mg, 0.34 mmol) were reacted in a manner similar to 184 to afford the title compound (463) (9.2 mg, 8%) as an off-white solid. MS (ESI) calculated for (C₂₇H₂₇F₃N₄O₃) [M+H]⁺, 513.2; found, 512.9. ¹H NMR (300 MHz, DMSO-d₆) δ 12.42 (s, 1H), 8.12-8.07 (m, 2H), 7.82 (s, 1H), 7.75 (d, J=7.8 Hz, 1H), 7.39-7.34 (m, 1H), 7.22 (s, 1H), 7.10 (d, J=7.5 Hz, 1H), 5.25 (s, 2H), 5.04 (br, 1H), 4.35-4.27 (m, 1H), 3.67-3.57 (m, 3H), 3.45-3.11 (m, 2H), 2.87-2.72 (m, 2H), 2.01-1.86 (m, 5H), 1.22 (d, J=6.9 Hz, 3H).

### Example 464: 6-((2R,4S)-4-hydroxy-2-methylpyrrolidine-1-carbonyl)-2-(3-((R)-1-(4-methyl-1H-pyrazol-3-yl)propan-2-yl)phenyl)-4-(trifluoromethyl)isoindolin-1-one (464)

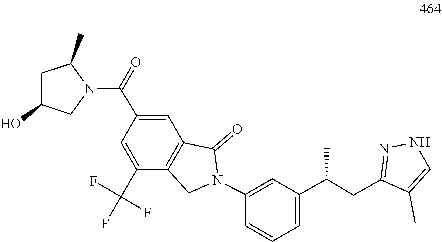 [SMILES: Cc1c[nH]nc1C[C@@H](C)c1cccc(N2Cc3c(cc(C(=O)N4C[C@@H](O)C[C@H]4C)cc3C(F)(F)F)C2=O)c1]

Step 1: Synthesis of (3S,5R)-5-methylpyrrolidin-3-ol hydrochloride. A mixture of (2R,4S)-tert-butyl 4-hydroxy-2-methylpyrrolidine-1-carboxylate (400 mg, 1.99 mmol) in HCl (10 mL, 4 M in dioxane) was stirred at rt for 3 h. The solvents were removed under vacuum to afford (3S, 5R)-5-methylpyrrolidin-3-ol hydrochloride (350 mg, crude) as a colorless solid, which was used without purification. MS (ESI) calculated for (C₅H₁₁NO) [M+H]⁺, 102.1; found, 102.0.

Step 2: Synthesis of 6-((2R,4S)-4-hydroxy-2-methylpyrrolidine-1-carbonyl)-2-(3-((R)-1-(4-methyl-1H-pyrazol-3-yl)propan-2-yl)phenyl)-4-(trifluoromethyl)isoindolin-1-one (464). (R)-2-(3-(1-(4-methyl-1H-pyrazol-3-yl)propan-2-yl)phenyl)-3-oxo-7-(trifluoromethyl)isoindoline-5-carboxylic acid (463, step 1) and (3S,5R)-5-methylpyrrolidin-3-ol hydrochloride (46.4 mg, 0.34 mmol) were reacted in a manner similar to 184 to give the title compound (36.1 mg, 30%) as an off-white solid. ¹H NMR (300 MHz, DMSO-d₆) δ 8.09-8.07 (m, 2H), 7.81 (s, 1H), 7.76-7.74 (m, 1H), 7.39-7.34 (m, 1H), 7.29 (s, 1H), 7.10 (d, J=7.8 Hz, 1H), 5.25 (s, 2H), 4.38-3.75 (m, 3H), 3.51-3.33 (m, 2H), 3.16-3.09 (m, 1H), 2.89-2.74 (m, 2H), 2.32-2.26 (m, 1H), 1.87 (s, 3H), 1.60-1.53 (m, 1H), 1.40 (d, J=6.3 Hz, 3H), 1.22 (d, J=6.9 Hz, 3H). MS (ESI) calculated for (C₂₈H₂₉F₃N₄O₃) [M+H]⁺, 527.2; found, 526.9.

### Example 465: 6-((S)-3-hydroxypyrrolidine-1-carbonyl)-4-(trifluoromethyl)-2-(3-((R)-1-(4-(trifluoromethyl)-1H-pyrazol-3-yl)propan-2-yl)phenyl)isoindolin-1-one (465)

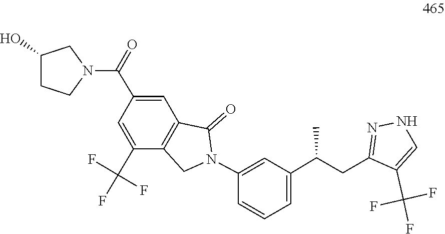 [SMILES: C[C@H](Cc1n[nH]cc1C(F)(F)F)c1cccc(N2Cc3c(cc(C(=O)N4CC[C@H](O)C4)cc3C(F)(F)F)C2=O)c1]

Step 1: Synthesis of (3R)-3-(3-nitrophenyl)butan-1-ol. To a mixture of LiAlH₄ (320 mg, 8.43 mmol) in THF (30 mL) was added a solution of ethyl (3H)-3-(3-nitrophenyl)butanoate (2.0 g, 8.4 mmol) in THF (5 mF) dropwise at 0° C. The mixture was stirred at 0° C. for 2 h. The reaction was then quenched by the addition of water (0.3 mF) and 15% NaOH (0.9 mF) and then filtered. The filtrate was evaporated under vacuum. The residue was purified by flash column chromatography with 20-50% EtOAc in petroleum ether to afford the title compound (1.0 g, 60%) as a light yellow oil.

Step 2: Synthesis of (3R)-3-(3-nitrophenyl)butanal. A mixture of (3R)-3-(3-nitrophenyl)butan-1-ol (2.5 g, 13 mmol) and IBX (9.5 g, 34 mmol) in EtOAc (120 mF) was heated at 80° C. for 4 h. The solids were filtered out. The filtrate was evaporated under vacuum. The residue was purified by flash column chromatography with 10-30% EtOAc in petroleum ether to afford the title compound (2.3 g, 92%) as a light yellow oil.

Step 3: Synthesis of (5R)-1,1,1-trifluoro-5-(3-nitrophenyl)hexan-3-one. To a sealed tube was added a mixture of 2,2,2-trifluoroethan-1-amine hydrochloride (2.6 g, 19 mmol) and NaNO₂ (1.7 g, 26 mmol) in CH₂Cl₂ (90 mF) and water (3.0 mF). The mixture was stirred at 0° C. for 1 h and then cooled to −70° C. for 10 min. Then a mixture of (3R)-3-(3-nitrophenyl)butanal (1.7 g, 8.8 mmol) and tetrachlorozirconium (3.3 g, 14 mmol) was added to the mixture. The mixture was stirred at −70° C. for 1.5 h before quenched by the addition of methanol (15 mL). The mixture was evaporated under vacuum. The residue was purified by flash column chromatography with 5-30% EtOAc in petroleum ether to afford the title compound (1.8 g, 74%) as a light yellow solid.

Step 4: Synthesis of (1Z,5R)-1-(dimethylamino)-5-(3-nitrophenyl)-2-(trifluoromethyl)hex-1-en-3-one. A mixture of (5R)-1,1,1-trifluoro-5-(3-nitrophenyl)hexan-3-one (1.88 g, 6.83 mmol) and DMF-DMA (1.90 g, 15.9 mmol) in toluene (50.0 mL) was heated at 100° C. for 4 h. The mixture was evaporated under vacuum. The residue was purified by flash column chromatography with 20-50% EtOAc in petroleum ether to give the title compound (1.3 g, 57%) as a light yellow oil. MS (ESI) calculated for (C₁₅H₁₇F₃N₂O₃) [M+H]⁺, 331.1, found, 330.8.

Step 5: Synthesis of 3-[(2R)-2-(3-nitrophenyl)propyl]-4-(trifluoromethyl)-1H-pyrazole. A mixture of (1Z,5R)-1-(dimethylamino)-5-(3-nitrophenyl)-2-(trifluoromethyl)hex-1-en-3-one (1.3 g, 4.0 mol) and hydrazine hydrate (820 mg, 13.1 mmol, 80%) in 1,4-dioxane (20.0 mL) was stirred at 70° C. for 16 h. The solid was filtered out. The filtrate was concentrated and purified by reverse phase flash column chromatography with 5-44% acetonitrile in water to afford the title compound (390 mg, 32%) as a light yellow oil. MS (ESI) calculated for (C₁₃H₁₂F₃N₃O₂) [M+H]⁺, 300.1, found, 299.7.

Step 6: Synthesis of 3-[(2R)-1-[4-(trifluoromethyl)-1H-pyrazol-3-yl]propan-2-yl]aniline. A mixture of 3-[(2R)-2-(3-nitrophenyl)propyl]-4-(trifluoromethyl)-1H-pyrazole (174 mg, 0.58 mmol) and Pd/C (70.0 mg, 0.66 mmol) in methanol (8.0 mL) was stirred at rt for 16 h under H₂. The solids were filtered out. The filtrate was evaporated under vacuum to afford the title compound (130 mg, crude) as a light yellow oil. MS (ESI) calculated for (C₁₃H₁₄F₃N₃) [M+H]⁺, 270.0, found, 269.8.

Step 7: Synthesis of 3-oxo-7-(trifluoromethyl)-2-[3-[(2R)-1-[4-(trifluoromethyl)-1H-pyrazol-3-yl]propan-2-yl]phenyl]-2,3-dihydro-1H-isoindole-5-carboxylic acid. The indolone formation reaction was carried out in a manner similar to 260, step 2 using 3-[(2R)-1-[4-(trifluoromethyl)-1H-pyrazol-3-yl]propan-2-yl]aniline (130 mg, 0.48 mmol) and 4-(bromomethyl)-3-(methoxycarbonyl)-5-(trifluoromethyl)benzoic acid (174 mg, 0.51 mmol) to afford the title compound (100 mg, 41%) as a colorless solid. MS (ESI) calculated for (C₂₃H₁₇F₆N₃O₃) [M+H]⁺, 498.1, found, 497.8.

Step 8: Synthesis of 6-[(3S)-3-hydroxypyrrolidine-1-carbonyl]-4-(trifluoromethyl)-2-[3-[(2R)-1-[4-(trifluoromethyl)-1H-pyrazol-3-yl]propan-2-yl]phenyl]-2,3-dihydro-1H-isoindol-1-one (465). 3-oxo-7-(trifluoromethyl)-2-[3-[(2R)-1-[4-(trifluoromethyl)-1H-pyrazol-3-yl]propan-2-yl]phenyl]-2,3-dihydro-1H-isoindole-5-carboxylic acid (110 mg, 0.22 mmol), (3S)-pyrrolidin-3-ol hydrochloride (59.8 mg, 0.48 mmol) were coupled in a manner similar to 184 to afford the title compound (37.0 mg, 29%) as a colorless solid. MS (ESI) calculated for (C₂₇H₂₄F₆N₄O₃) [M+H]⁺, 567.2, found, 566.8. ¹H NMR (400 MHz, DMSO-d₆) δ 8.14-8.09 (m, 2H), 7.96 (s, 1H), 7.86 (s, 1H), 7.75 (d, J=8.0 Hz, 1H), 7.40-7.36 (m, 1H), 7.09 (d, J=7.6 Hz, 1H), 5.25 (s, 2H), 4.35-4.27 (m, 1H), 3.66-3.55 (m, 2H), 3.45-3.42 (m, 2H), 3.24-3.20 (m, 2H), 3.05-2.91 (m, 2H), 2.01-1.83 (m, 2H), 1.24 (d, J=6.8 Hz, 3H).

### Example 466: 6-(1-((S)-3-fluoropyrrolidin-1-yl)ethyl)-2-(3-((1S,2R)-2-(4-methyl-4H-1,2,4-triazol-3-yl)cyclopropyl)phenyl)-4-(trifluoromethyl)isoindolin-1-one (466)

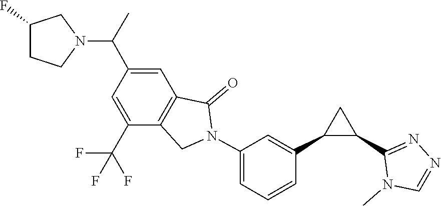 [SMILES: CC(c1cc2c(c(C(F)(F)F)c1)CN(c1cccc([C@H]3C[C@H]3c3nncn3C)c1)C2=O)N1CC[C@H](F)C1]

Step 1: Synthesis of 6-acetyl-2-(3-((1S,2R)-2-(4-methyl-4H-1,2,4-triazol-3-yl)cyclopropyl)phenyl)-4-(trifluoromethyl)isoindolin-1-one. The methyl ketone was synthesized according to the procedure for 459, step 1 using 6-bromo-2-(3-((1S,2R)-2-(4-methyl-4H-1,2,4-triazol-3-yl)cyclopropyl)phenyl)-4-(trifluoromethyl)isoindolin-1-one (508 mg, 1.06 mmol, 1.0 eq.) as the reactant to afford the title compound in 416 mg (89%) yield.

Step 2: Synthesis of 6-(1-((S)-3-fluoropyrrolidin-1-yl)ethyl)-2-(3-((1S,2R)-2-(4-methyl-4H-1,2,4-triazol-3-yl)cyclopropyl)phenyl)-4-(trifluoromethyl)isoindolin-1-one. Reductive amination of the methyl ketone was carried out according to the procedure for 459, step 2 using 6-acetyl-2-(3-((1S,2R)-2-(4-methyl-4H-1,2,4-triazol-3-yl)cyclopropyl)phenyl)-4-(trifluoromethyl)isoindolin-1-one (50 mg, 0.11 mmol, 1.0 eq.) and (S)-3-fluoropyrrolidine hydrochloride (58 mg, 0.46 mmol, 4.1 eq.) as reactants to afford the title compound (31 mg, 53% yield) as a colorless solid. ¹H NMR (500 MHz, DMSO-d6) δ 8.13 (s, 1H), 7.98 (s, 1H), 7.93 (d, J=2.0 Hz, 1H), 7.69 (ddd, J=8.1, 2.3, 1.0 Hz, 1H), 7.53 (s, 1H), 7.14 (t, J=7.9 Hz, 1H), 6.73 (d, J=7.7 Hz, 1H), 5.30-5.09 (m, 1H), 5.09-4.95 (m, 2H), 3.60 (dq, J=13.1, 6.7 Hz, 1H), 3.41 (s, 3H), 2.98-2.88 (m, 1H), 2.71-2.60 (m, 2H), 2.41-2.32 (m, 1H), 2.29-2.01 (m, 1H), 1.89 (q, J=5.9 Hz, 1H), 1.58 (td, J=8.5, 5.0 Hz, 1H), 1.35 (dd, J=6.6, 2.2 Hz, 3H); LCMS: C₂₇H₂₇F₄N₅O requires: 513, found: m/z=514 [M+H]⁺.

### Example 467: (S)-2-(3-(1,1-difluoro-1-(4-methyl-4H-1,2,4-triazol-3-yl)propan-2-yl)phenyl)-4-(trifluoromethyl)isoindolin-1-one (467a)

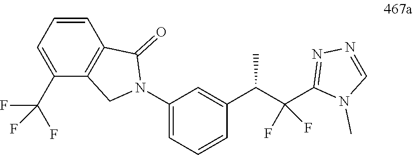 [SMILES: C[C@@H](c1cccc(N2Cc3c(cccc3C(F)(F)F)C2=O)c1)C(F)(F)c1nncn1C]

Step 1: Synthesis of 3-(3-nitrophenyl)-2-oxobutanehydrazide. To a solution of ethyl 3-(3-nitrophenyl)-2-oxobutanoate (8.1 g, 31.87 mmol; Example 292 step 3) in ethanol (80 mL) was added hydrazine hydrate (18.0 g, 0.32 mol) dropwise at 0° C. The solution was stirred at rt for 3 h, and then concentrated. The residue was purified by flash column chromatography with 0˜9% methanol in dichloromethane to afford the title compound (6.1 g, 80%) as a yellow solid. MS (ESI) calculated for (C₁₀H₁₁N₃O₄) [M+H]⁺, 238.1; found, 238.2.

Step 2: Synthesis of N-methyl-2-(3-(3-nitrophenyl)-2-oxobutanoyl)hydrazinecarbothioamide. To a solution of 3-(3-nitrophenyl)-2-oxobutanehydrazide (6.1 g, 25.63 mmol) in THF (250 mL) was added isothiocyanatomethane (2.3 g, 31.51 mmol). The solution was stirred at 55° C. for 2 h, and then concentrated. The residue was purified by flash column chromatography with 0˜5% methanol in dichloromethane to afford the title compound (7.5 g, 92%) as a yellow syrup. MS (ESI) calculated for (C₁₂H₁₄N₄O₄S) [M+H]⁺, 311.1; found, 311.1.

Step 3: Synthesis 1-(5-mercapto-4-methyl-4H-1,2,4-triazol-3-yl)-2-(3-nitrophenyl)propan-1-one. A solution of N-methyl-2-(3-(3-nitrophenyl)-2-oxobutanoyl)hydrazinecarbothioamide (7.5 g, 24.19 mmol) in sodium hydroxide (1 N, 242 mL) was stirred at 45° C. for 3 h. The pH value was adjusted to 3 by the addition of HCl (3 N). The solution was extracted with dichloromethane. All the organic layers were combined, washed with brine, dried, and concentrated. The residue was purified by flash column chromatography with 07% methanol in dichloromethane to afford the title compound (4.0 g, 56%) as a yellow solid. MS (ESI) calculated for (C₁₂H₁₂N₄O₃S) [M+H]⁺, 293.1; found, 293.1.

Step 4: Synthesis of 1-(4-methyl-4H-1,2,4-triazol-3-yl)-2-(3-nitrophenyl)propan-1-one. To a solution of l-(5-mercapto-4-methyl-4H-1,2,4-triazol-3-yl)-2-(3-nitrophenyl)propan-1-one (4.0 g, 13.70 mmol) in dichloromethane (14 mL) and AcOH (7 mL) was added H₂O₂ (28% aq., 5.0 g) dropwise at 0° C. The mixture was stirred at 25° C. for 1.5 h. the reaction was quenched by the addition of saturated NaHCO₃ aqueous solution and extracted with dichloromethane. All the organic layers were combined, washed with brine, dried, and concentrated. The residue was purified by flash column chromatography with 0˜13% methanol in dichloromethane to afford the title compound (1.8 g, 51%) as a brown syrup. MS (ESI) calculated for (C₁₂H₁₂N₄O₃) [M+H]⁺, 261.1; found, 261.2.

Step 5: Synthesis of 3-(1,1-difluoro-2-(3-nitrophenyl)propyl)-4-methyl-4H-1,2,4-triazole. A mixture of 1-(4-methyl-4H-1,2,4-triazol-3-yl)-2-(3-nitrophenyl)propan-1-one (1.8 g, 6.92 mmol) and DAST (15 mL) was stirred at 60° C. for 6 h. the reaction was quenched by the addition of saturated sodium bicarbonate aqueous solution at 0° C. The solution was extracted with dichloromethane. All the organic layers were combined, washed with brine, dried, and concentrated. The residue was purified by flash column chromatography with 0-20% methanol in dichloromethane to afford title compound (1.4 g), which was purified by Prep-HPLC to afford the title compound (330 mg, 17%) as a colorless solid. MS (ESI) calculated for (C₁₂H₁₂F₂N₄O₂) [M+H]⁺, 283.1; found, 283.1.

Step 6: Synthesis of 3-(1,1-difluoro-1-(4-methyl-4H-1,2,4-triazol-3-yl)propan-2-yl)aniline. To a solution of 3-(1,1-difluoro-2-(3-nitrophenyl)propyl)-4-methyl-4H-1,2,4-triazole (150 mg, 0.53 mmol) in methanol (5 mL) was added Pd/C (10%, 50 mg). The solution was stirred at 25° C. for 16 h under H₂ (2 atm). The solids were filtered out. The mixture was concentrated to afford the title compound (120.2 mg, crude) as a yellow syrup, which was used without purification. MS (ESI) calculated for (C₁₂H₁₄F₂N₄) [M+H]⁺, 253.1; found, 253.2.

Step 7: Synthesis of 2-(3-(1,1-difluoro-1-(4-methyl-4H-1,2,4-triazol-3-yl)propan-2-yl)phenyl)-4-(trifluoromethyl)isoindolin-1-one. To a solution of 3-(1,1-difluoro-1-(4-methyl-4H-1,2,4-triazol-3-yl)propan-2-yl)aniline (45.1 mg, 0.18 mmol) and methyl 2-(bromomethyl)-3-(trifluoromethyl)benzoate (56.1 mg, 0.21 mmol) in ethanol (15 mL) was added TEA (115.1 mg, 1.14 mmol). The solution was stirred at 80° C. for 3 h, and then concentrated. The residue was purified by Prep-HPLC to afford the title compound (9.1 mg, 26%) as a colorless solid. MS (ESI) calculated for (C₂₁H₁₇F₅N₄O) [M+H]⁺, 437.1; found, 436.9.

Step 8: (S)-2-(3-(1,1-difluoro-1-(4-methyl-4H-1,2,4-triazol-3-yl)propan-2-yl)phenyl)-4-(trifluoromethyl)isoindolin-1-one (467a) and (R)-2-(3-(1,1-difluoro-1-(4-methyl-4H-1,2,4-triazol-3-yl)propan-2-yl)phenyl)-4-(trifluoromethyl)isoindolin-1-one (467b). Racemic 2-[3-[1,1-difluoro-1-(4-methyl-4H-1,2,4-triazol-3-yl)propan-2-yl]phenyl]-4-(trifluoromethyl)-2,3-dihydro-1H-isoindol-1-one (150 mg) was purified by Chiral-Prep-HPLC with the following conditions: [ChiralPak IE, 2*25 cm, 5 pm; Mobile Phase A: Hex (8 mmol/L, NH₃.methanol), Mobile Phase B: EtOH; Flow rate: 15 mL/min; Gradient: 50% B to 50% B in 24 min] to afford:

(S)-2-(3-(1,1-difluoro-1-(4-methyl-4H-1,2,4-triazol-3-yl)propan-2-yl)phenyl)-4-(trifluoromethyl)isoindolin-1-one (467a): (53.5 mg, colorless solid, shorter retention time) MS (ESI) calculated for (C₂₁H₁₇F₅N₄O), 436; found, 437 [M+H]⁺. ¹H NMR (400 MHz, Methanol-d₄) δ 8.44 (s, 1H), 8.12 (d, J=8.0 Hz, 1H), 7.99 (d, J=8.0 Hz, 1H), 7.85 (s, 1H), 7.81-7.77 (m, 2H), 7.44 (t, J=8.0 Hz, 1H), 7.20 (d, J=7.6 Hz, 1H), 5.16 (s, 2H), 4.07-3.94 (m, 1H), 3.57 (s, 3H), 1.64 (d, J=7.2 Hz, 3H). ¹⁹F NMR (376 MHz, MeOD) δ-63.00, −98.70, −99.42, −102.69, −103.41.

(R)-2-(3-(1,1-difluoro-1-(4-methyl-4H-1,2,4-triazol-3-yl)propan-2-yl)phenyl)-4-(trifluoromethyl)isoindolin-1-one (467b): (53.8 mg, colorless solid, longer retention time) MS (ESI) calculated for (C₂₁H₁₇F₅N₄O), 436; found, 437 [M+H]⁺. ¹H NMR (400 MHz, Methanol-d₄) δ 8.44 (s, 1H), 8.12 (d, J=8.0 Hz, 1H), 7.99 (d, J=7.6 Hz, 1H), 7.85 (s, 1H), 7.81-7.77 (m, 2H), 7.44 (t, J=8.0 Hz, 1H), 7.20 (d, J=7.6 Hz, 1H), 5.16 (s, 2H), 4.08-3.94 (m, 1H), 3.57 (s, 3H), 1.64 (d, J=7.2 Hz, 3H). ¹⁹F NMR (376 MHz, MeOD) 5-63.00, −98.70, −99.42, −102.69, −103.42.

### Example 468: 2-(3-ethoxy-5-{3-[(4-methyl-4H-1,2,4-triazol-3-yl)methyl]oxetan-3-yl}phenyl)-6-{[(3×)-3-fluoropyrrolidin-1-yl]methyl}-4-(trifluoromethyl)-2,3-dihydro-1H-isoindol-1-one (468)

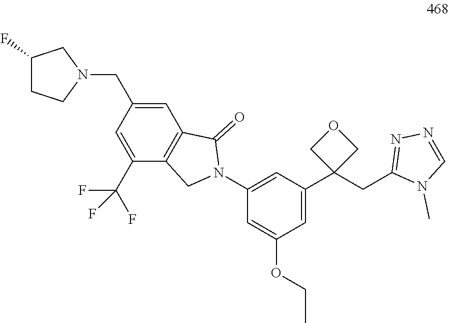 [SMILES: CCOc1cc(N2Cc3c(cc(CN4CC[C@H](F)C4)cc3C(F)(F)F)C2=O)cc(C2(Cc3nncn3C)COC2)c1]

Step 1: Synthesis of ethyl 2-[3-(3-bromo-5-ethoxyphenyl)oxetan-3-yl]acetate. To a stirring solution of bis((1Z, 5Z)-cycloocta-1,5-diene): bis(chlororhodium) (173 mg, 0.35 mmol) and ethyl 2-(oxetan-3-ylidene)acetate (1.0 g, 7.0 mmol) in dioxane (4 mL) was added potassium hydroxide (0.79 g, 14.0 mmol) in water (0.6 mL). The solution was stirred for 5 min and then (3-bromo-5-ethoxyphenyl)boronic acid (1.7 g, 7.0 mmol) was added. The reaction was heated to 40° C. for about 18 hours under nitrogen, cooled to rt, and then concentrated. The reaction was diluted with water and the aqueous layer was extracted with EtOAc. The combined organic layers were washed, dried, filtered, and concentrated in vacuo. The crude was purified using flash column chromatography 20-60% EtOAc/hexanes to yield the title compound (1.4 g, 58%).

Step 2: Synthesis of 2-[3-(3-bromo-5-ethoxyphenyl)oxetan-3-yl]acetohydrazide. To a stirring solution of ethyl 2-[3-(3-bromo-5-ethoxyphenyl)oxetan-3-yl]acetate (1.36 g, 3.95 mmol) in ethanol (40 mL) was added hydrazine hydrate (4.0 mL, 40.0 mmol). The mixture was stirred at 80° C. for about 12 hours, cooled to rt, and then concentrated. The reaction was diluted with water and extracted with EtOAc. The combined organic layers were washed were dried, filtered, and concentrated under reduced pressure. The crude was used in the next step without further purification.

Step 3: Synthesis of 5-{[3-(3-bromo-5-ethoxyphenyl)oxetan-3-yl]methyl}-4-methyl-1,2,4-triazole-3-thiol. To a solution of 2-[3-(3-bromo-5-ethoxyphenyl)oxetan-3-yl]acetohydrazide (1.30 g, 3.95 mmol) in THF (40 mL, g, 3.95 mmol) was added isothiocyanatomethane (0.4 mL, 433 mg, 5.92 mmol). The mixture was stirred at rt for 4 h. potassium hydroxide (1 M, 30 mL) was added and the reaction was stirred overnight. The solution was partitioned between dichloromethane and water. The layers were dried, filtered, and concentrated. The crude material was purified by silica gel column chromatography using methanol in dichloromethane 0-10% to the title compound (1.10 g, 73%) as a clear oil.

Step 4: Synthesis of 3-{[3-(3-bromo-5-ethoxyphenyl)oxetan-3-yl]methyl}-4-methyl-1,2,4-triazole. A solution of crude 5-{[3-(3-bromo-5-ethoxyphenyl)oxetan-3-yl]methyl}-4-methyl-4H-1,2,4-triazole-3-thiol (1.10 g, 2.86 mmol) in dichloromethane (5 mL) and acetic acid (2.5 mL) was treated with hydrogen peroxide (30% aqueous solution, 1 mL) dropwise. The mixture was stirred at rt for 16 h and evaporated to dryness. The solution was partitioned between dichloromethane and sodium bicarbonate. The aqueous layer was extracted with dichloromethane. The combined organic layers were dried, filtered, and concentrated under reduced pressure. The crude material was purified by silica gel column chromatography using methanol in dichloromethane 0-10% to give crude material which was purified by silica gel column chromatography using methanol in dichloromethane 0-10% to give the title compound (820 mg, 81%).

Step 5: Synthesis of 2-(3-ethoxy-5-{3-[(4-methyl-4H-1,2,4-triazol-3-yl)methyl]oxetan-3-yl}phenyl)-6-{[(3×)-3-fluoropyrrolidin-1-yl]methyl}-4-(trifluoromethyl)-2,3-dihydro-1H-isoindol-1-one. The coupling reaction was carried out in a similar fashion to Example 168, step 1 using 6-{(3S)-3-fluoropyrrolidin-1-yl methyl}-4-(trifluoromethyl)-2,3-dihydro-1H-isoindol-1-one (85 mg, 0.28 mmol) and 3-{[3-(3-bromo-5-ethoxyphenyl)oxetan-3-yl]methyl}-4-methyl-4H-1,2,4-triazole (99 mg, 0.28 mmol) as reactants afford the title compound (43.00 mg, 27%) as an off-white solid: ¹H NMR (500 MHz, DMSO-d6) δ 8.22 (s, 1H), 7.98 (d, J=20.3 Hz, 2H), 7.55 (t, J=2.1 Hz, 1H), 6.94 (t, J=1.6 Hz, 1H), 6.29 (t, J=1.8 Hz, 1H), 5.23 (d, J=55.8 Hz, 1H), 5.09 (s, 2H), 4.91 (dd, J=31.1, 6.0 Hz, 4H), 3.96 (q, J=6.9 Hz, 2H), 3.85 (s, 2H), 2.95 (s, 3H), 2.88-2.77 (m, 2H), 2.73-2.60 (m, 1H), 2.39 (q, J=7.8 Hz, 1H), 2.18 (ddq, J=27.8, 14.2, 6.9 Hz, 2H), 1.92 (ddt, J=29.8, 14.2, 7.1 Hz, 2H), 1.32 (t, J=7.0 Hz, 3H); LCMS: C₂₉H₃₁F₄N₅O₃ requires: 573, found: m/z=574 [M+H]⁺.

### Example 469: 2-cyclopropyl-N-{6-ethoxy-4-[(2R)-1,1,2-trifluoro-1-(4-methyl-4H-1,2,4-triazol-3-yl)propan-2-yl]pyridin-2-yl}-6-methylpyrimidine-4-carboxamide (469)

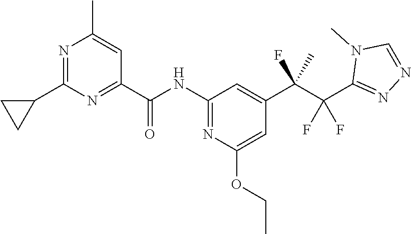 [SMILES: CCOc1cc([C@@](C)(F)C(F)(F)c2nncn2C)cc(NC(=O)c2cc(C)nc(C3CC3)n2)n1]

Step 1: Synthesis of ethyl 3-(2,6-dichloropyridin-4-yl)-2,2-difluoro-3-hydroxybutanoate. To a solution of Zn (148 g, 2.27 mol, 3.6 eq.) and ethyl 2-bromo-2,2-difluoro-acetate (12.8 g, 63.1 mmol, 8.1 mL, 0.1 eq.) in THF (1200 mL) was added DIBAL-H (1 M, 25 mL, 0.04 eq.). The mixture was stirred at 30° C. for 1 h, then a solution of 1-(2,6-dichloropyridin-4-yl)ethan-1-one (WO2015026792) (120 g, 631 mmol, 1.0 eq) and ethyl 2-bromo-2,2-difluoro-acetate (128 g, 631 mmol, 81 mL, 1.0 eq.) in THF (300 mL) was added drop-wise at 40° C. and stirred at 40° C. for 3 hours. Four batches of this reaction were prepared in parallel and combined prior to the workup. The combined mixture was filtered and the filtrate was poured into ammonium chloride. The aqueous layer was extracted with EtOAc. The organic layer was combined and concentrated to give the crude product. The crude product was purified by silica gel chromatography 0-10% petroleum ether/ethyl acetate to give the title compound (415 g, 1.32 mol, 61%, 3.4 eq.) as a yellow oil.

Step 2: Synthesis of 3-(2,6-dichloropyridin-4-yl)-2,2-difluoro-3-hydroxybutanehydrazide. To a solution of 3-(2,6-dichloropyridin-4-yl)-2,2-difluoro-3-hydroxybutanoate (200 g, 636 mmol, 1.0 eq.) in THF (1000 mL) was added hydrazine hydrate (338 g, 6.62 mol, 98% purity, 10.4 eq.). The mixture was stirred at 25° C. for 16 hr. The mixture was poured into water and extracted with EtOAc. The organic layers were washed with brine, dried, and concentrated to afford the title compound, which was used without purification.

Step 3: Synthesis of N′-((I2-azanyl)carbonothioyl)-3-(2,6-dichloropyridin-4-yl)-2,2-difluoro-3-hydroxybutanehydrazide. To a solution of 3-(2,6-dichloropyridin-4-yl)-2,2-difluoro-3-hydroxybutanehydrazide (230 g, 766 mmol, 1.0 eq.) in THF (1100 mL) was added methylimino(thioxo)methane (112 g, 1.53 mol, 105 mL, 2.0 eq.). The mixture was stirred at 70° C. for 2 hr. The mixture was concentrated and filtered. The filter cake was collected to give the title compound (280 g, crude) as a colorless solid.

Step 4: Synthesis of 2-(2,6-dichloropyridin-4-yl)-1,1-difluoro-1-(5-mercapto-4-methyl-4H-1,2,4-triazol-3-yl)propan-2-ol. A solution of N′-((I2-azanyl)carbonothioyl)-3-(2,6-dichloropyridin-4-yl)-2,2-difluoro-3-hydroxybutanehydrazide in NaOH (1 M, 2.80 L, 3.7 eq.) was stirred at 50° C. for 2 hours. The mixture was poured into HCl (1 M, 3000 mL) and filtered. The filter cake was collected to give the title compound (350.0 g, crude) as a colorless solid.

Step 5: Synthesis of 2-(2,6-dichloropyridin-4-yl)-1,1-difluoro-1-(4-methyl-4H-1,2,4-triazol-3-yl)propan-2-ol. To a solution of 2-(2,6-dichloropyridin-4-yl)-1,1-difluoro-1-(5-mercapto-4-methyl-4H-1,2,4-triazol-3-yl)propan-2-ol (350 g, 985 mmol, 1.0 eq.) in dichloromethane (1500 mL) was added a solution of H₂O₂ (335 g, 2.9 mol, 284 mL, 30.0% purity, 3.0 eq.) in HOAc (88 g, 1.48 mol, 84.5 mL, 1.5 eq.) at 35° C. and stirred for 16 hours. The mixture was poured into saturated sodium bicarbonate solution and filtered. The filter cake was collected and washed with saturated sodium sulfate solution, the title compound (110 g, crude) was obtained as a yellow solid:

Step 6: Synthesis of (R)-2,6-dichloro-4-(1,1,2-trifluoro-1-(4-methyl-4H-1,2,4-triazol-3-yl)propan-2-yl)pyridine. 2-(2,6-dichloropyridin-4-yl)-1,1-difluoro-1-(4-methyl-4H-1,2,4-triazol-3-yl)propan-2-ol (71 g, 219 mmol, 1.0 eq.) and HF (71.5 g, 3.57 mmol, 65.0 mL, 100% purity, 16.2 eq.) was kept in a stainless steel autoclave. Tetrafluoro-sulfane (71.2 g, 659 mmol, 3.0 eq.) was added in a stainless steel autoclave at −78° C. The mixture was stirred at 10-20° C. for 14 hours. The reaction was reacted in 0.9 MPa. To the reaction mixture was added saturate aqueous sodium bicarbonate to adjust the pH to 6-8, then extracted with EtOAc, dried, and concentrated under to give the residue. The residue was purified by silica column chromatography 0-10% petroleum ether/ethyl acetate to give 2,6-dichloro-4-(1,1,2-trifluoro-1-(4-methyl-4H-1,2,4-triazol-3-yl)propan-2-yl)pyridine as a yellow solid. The racemic product was separated by prep-chiral-HPLC with the following condition: daicel chiralcel od (250 mm*30 mm, 10 um); mobile phase: [0.1% NH₃, water, IPA]; B %: 40%-40%, 3 min; 2000 min afforded:

(S)-2,6-dichloro-4-(1,1,2-trifluoro-1-(4-methyl-4H-1,2,4-triazol-3-yl)propan-2-yl)pyridine (11.8 g): ¹H NMR (400 MHz, DMSO-d6) δ: 8.71 (s, 1H), 7.68 (s, 2H), 3.75 (s, 3H), 1.99 (d, J=24.4 Hz, 3H).

(R)-2,6-dichloro-4-(1,1,2-trifluoro-1-(4-methyl-4H-1,2,4-triazol-3-yl)propan-2-yl)pyridine (12.1 g): ¹H NMR (400 MHz, DMSO-d6) δ: 8.72 (s, 1H), 7.69 (s, 2H), 3.75 (s, 3H), 1.99 (d, J=24.4 Hz, 3H).

Step 7: Synthesis of (R)-2-chloro-6-ethoxy-4-(1,1,2-trifluoro-1-(4-methyl-4H-1,2,4-triazol-3-yl)propan-2-yl)pyridine. To a stirring solution of ethanol (15 mL, 140 mg, 3.08 mmol) at 0° C. was added sodium hydride (246 mg, 6.15 mmol). The solution was stirred for 10 min and then (R)-2,6-dichloro-4-(1,1,2-trifluoro-1-(4-methyl-4H-1,2,4-triazol-3-yl)propan-2-yl)pyridine (1.0 g, 3.1 mmol) was added. The reaction was refluxed for about 2 h and then cooled to rt. The crude material was concentrated and purified by silica gel column chromatography using methanol in dichloromethane 0-10% to give the title compound (800 mg, 78%) as a colorless solid.

Step 8: Synthesis of 2-cyclopropyl-6-methylpyrimidine-4-carboxamide. To a solution of 2-cyclopropyl-6-methylpyrimidine-4-carboxylic acid (2.0 g, 11.2 mmol) in dichloromethane (20 mL, 0.95 g, 11.2 mmol) at 0° C. was added 2-methylpropyl carbonochloridate (1.6 mL, 1.7 g, 12.3 mmol). The solution was stirred for 1 h at 0° C. NH₄OH was added and the solution was stirred at rt for 12 h. The solution was partitioned between water and dichloromethane. The organic layer was dried, filtered, and concentrated to give the title compound (492 mg, 25%).

Step 9: Synthesis of 2-cyclopropyl-N-{6-ethoxy-4-[(2R)-1,1,2-trifluoro-1-(4-methyl-4H-1,2,4-triazol-3-yl)propan-2-yl]pyridin-2-yl}-6-methylpyrimidine-4-carboxamide. The coupling reaction was carried out in a similar fashion as for Example 168, step 1 using 2-cyclopropyl-6-methylpyrimidine-4-carboxamide (50 mg, 0.28 mmol, 1.0 eq.) and 2-chloro-6-ethoxy-4-[1,1,2-trifluoro-1-(4-methyl-4H-1,2,4-triazol-3-yl)propan-2-yl]pyridine (95 mg, 0.28 mmol, 1.0 eq.) as reactants afford the title compound (63 mg, 47%) as an off-white solid: ¹H NMR (500 MHz, DMSO-d6) δ 10.19 (s, 1H), 8.61 (s, 2H), 7.80 (s, 1H), 7.70 (s, 1H), 6.60 (d, J=1.2 Hz, 1H), 4.28 (q, J=7.0 Hz, 2H), 3.60 (s, 3H), 2.48 (s, 2H), 2.26 (td, J=7.3, 3.7 Hz, 2H), 1.88 (d, J=24.3 Hz, 3H), 1.28 (t, J=7.0 Hz, 3H), 1.13-0.94 (m, 4H); LCMS: C₂₂H₂₄F₃N₇O₂ requires: 475, found: m/z=476 [M+H]⁺.

### Example 470: 2-{3-[(1R,3R)-3-(hydroxymethyl)-1-[(4-methyl-4H-1,2,4-triazol-3-yl)methyl]cyclobutyl]phenyl}-4-(trifluoromethyl)-2,3-dihydro-1H-isoindol-1-one (470)

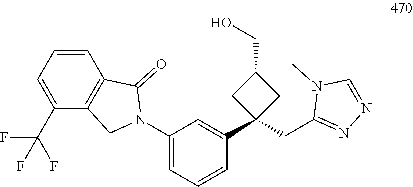 [SMILES: Cn1cnnc1C[C@]1(c2cccc(N3Cc4c(cccc4C(F)(F)F)C3=O)c2)C[C@H](CO)C1]

Step 1: Synthesis of ethyl 2-[3-(hydroxymethyl)cyclobutylidene]acetate. To a stirring solution of ethyl 2-(diethoxyphosphoryl)acetate (30 mL, 33.6 g, 149 mmol) in THF (500 mF) at 0° C., was added sodium hydride (5.0 g, 124 mmol). The reaction stirred at rt for 30 min, then a solution of 3-(hydroxymethyl)cyclobutan-1-one (5.00 g, 50 mmol) in THF (50 mF) was added and the reaction was allowed to proceed for 12 h. The mixture was concentrated, then purified by flash column chromatography eluting with 0-40% EtOAc/hexanes to give the title compound (5.50 g, 65%).

Step 2: Synthesis of ethyl 2-[3-(hydroxymethyl)-1-(3-nitrophenyl)cyclobutyl]acetate. Ethyl 2-[3-(hydroxymethyl)cyclobutylidene]acetate (4.00 g, 23.5 mmol) and bis((7Z, 5Z)-cycloocta-1,5-diene); bis(chlororhodium) (0.58 g, 1.18 mmol) were added to a mixture of (3-nitrophenyl)boronic acid (7.9 g, 47 mmol) in nitrogen sparged dioxane (150 mL) and 1 M potassium hydroxide (50 mL) under nitrogen atmosphere. The reaction was allowed to proceed at 40° C. for about 18 hours under nitrogen, cooled to rt, and then concentrated. The reaction was diluted with water and the aqueous layer was extracted with EtOAc. The combined organic layers were washed with brine and dried, filtered, and concentrated in vacuo. The crude was purified using flash column chromatography 20-80% EtOAc/hexanes to give the title compound (3.20 g, 46%).

Step 3: Synthesis of ethyl 2-[1-(3-aminophenyl)-3-(hydroxymethyl)cyclobutyl]acetate. To a stirred solution of ethyl 2-[3-(hydroxymethyl)-1-(3-nitrophenyl)cyclobutyl]acetate (700 mg, 2.3 mmol) and ammonium hydrochloride (1.28 g, 23.8 mmol) in ethanol (20 mL) and water (5 mL) was added iron (0.67 g, 11.9 mmol) in portions. The mixture was heated to about 80° C. for 12 h. The solution was filtered and concentrated to afford the title compound.

Step 4: Synthesis of ethyl 2-[3-(hydroxymethyl)-1-{3-[1-oxo-4-(trifluoromethyl)-3H-isoindol-2-yl]phenyl}cyclobutyl]acetate. Ethyl 2-[1-(3-aminophenyl)-3-(hydroxymethyl)cyclobutyl]acetate (628 mg, 2.38 mmol) and silver nitrate (810 mg, 4.77 mmol) were dissolved in methanol (20 mL) and water (5 mL). The solution was cooled to 0° C. and then methyl 2-(bromomethyl)-3-(trifluoromethyl)benzoate (708 mg, 2.38 mmol) in methanol was added dropwise. The solution was stirred for 2 h at 0° C. and then 12 hours at rt. The solution was partitioned between 15% IPA/chloroform and saturated sodium bicarbonate. The layers were filtered through Celite and then separated. The filtrate was extracted with 15% IPA/chloroform. The combined layers were dried, filtered, and concentrated. The crude material was purified by silica gel column chromatography using methanol in dichloromethane 0-10% to give the title compound (520 mg, 49% over two steps).

Step 5: Synthesis of 2-[3-(hydroxymethyl)-1-{3-[1-oxo-4-(trifluoromethyl)-2,3-dihydro-1H-isoindol-2-yl]phenyl}cyclobutyl]acetohydrazide. To a stirring solution of ethyl 2-[3-(hydroxymethyl)-1-{3-[1-oxo-4-(trifluoromethyl)-2,3-dihydro-1H-isoindol-2-yl]phenyl}cyclobutyl]acetate (400 mg, 0.89 mmol) in ethanol (10 mL) was added hydrazine hydrate (0.1 mL, 90 mg, 0.89 mmol). The mixture was stirred at 80° C. for about 12 hours, cooled to rt, and then concentrated. The reaction was diluted with water and extracted with EtOAc. The combined organic layers were washed were dried, filtered, and concentrated under reduced pressure.

Step 6: Synthesis of 2-{3-[3-(hydroxymethyl)-1-[(4-methyl-5-sulfanyl-4H-1,2,4-triazol-3-yl)methyl]-cyclobutyl]phenyl}-4-(trifluoromethyl)-2,3-dihydro-1H-isoindol-1-one. To a solution of 2-[3-(hydroxymethyl)-1-{3-[1-oxo-4-(trifluoromethyl)-2,3-dihydro-1H-isoindol-2-yl]phenyl}cyclobutyl]-aceto-hydrazide (385 mg, 0.89 mmol) in THE (10 mL) was added isothiocyanatomethane (0.12 mL, 1.78 mmol). The mixture was stirred for 4 h, and then evaporated in vacuo. The crude was dissolved in THF (10 mL) and potassium hydroxide (1M, 10 mL), and stirred for 12 h. The solution was partitioned between 15% IPA/chloroform and saturated ammonium chloride. The organic layer was dried, filtered, and concentrated.

Step 7: Synthesis of 2-{3-[(1r,3r)-3-(hydroxymethyl)-1-[(4-methyl-1,2,4-triazol-3-yl)methyl]cyclobutyl]phenyl}-4-(trifluoromethyl)-3H-isoindol-1-one. A solution of crude 2-{3-[3-(hydroxymethyl)-1-[(4-methyl-5-sulfanyl-4H-1,2,4-triazol-3-yl)methyl]cyclobutyl]phenyl}-4-(trifluoromethyl)-2,3-dihydro-1H-isoindol-1-one (434 mg, 0.89 mmol) in dichloromethane (5 mL) and acetic acid (2.5 mL) was treated with hydrogen peroxide (30% aqueous solution, 1 mL) dropwise. The mixture was stirred at rt for 16 h and evaporated to dryness. The crude material was purified by silica gel column chromatography using methanol in dichloromethane 0-10% to give the title compound (28 mg, 6.9% over 3 steps). ¹H NMR (500 MHz, DMSO-d6) δ 8.15 (s, 1H), 8.07 (dd, J=19.3, 7.7 Hz, 2H), 7.84-7.77 (m, 2H), 7.63 (t, J=2.0 Hz, 1H), 7.34 (t, J=7.9 Hz, 1H), 6.93 (dt, J=7.8, 1.2 Hz, 1H), 5.15 (s, 2H), 4.55 (t, J=5.3 Hz, 1H), 3.44 (t, J=5.7 Hz, 2H), 3.32 (s, 3H), 2.79 (s, 2H), 2.49-2.45 (m, 2H), 2.35-2.26 (m, 2H), 2.18 (h, J=7.3 Hz, 1H); LCMS: C₂₄H₂₃F₃N₄O₂ requires: 456, found: m/z=457 [M+H],

### Example 471: (R)-2-(3-(1,1,1-trifluoro-3-(4-methyl-4H-1,2,4-triazol-3-yl)propan-2-yl)phenyl)-4-(trifluoromethyl)isoindolin-1-one (471a) and (S)-2-(3-(1,1,1-trifluoro-3-(4-methyl-4H-1,2,4-triazol-3-yl)propan-2-yl)phenyl)-4-(trifluoromethyl)isoindolin-1-one (471b)

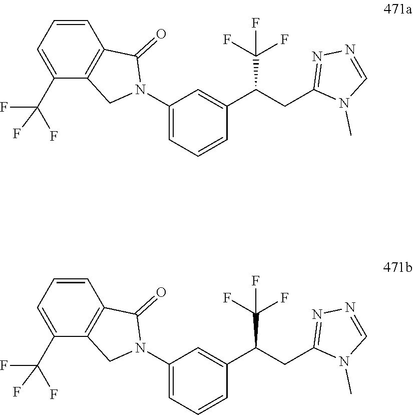 [SMILES: Cn1cnnc1C[C@H](c1cccc(N2Cc3c(cccc3C(F)(F)F)C2=O)c1)C(F)(F)F ; Cn1cnnc1C[C@@H](c1cccc(N2Cc3c(cccc3C(F)(F)F)C2=O)c1)C(F)(F)F]

Racemate 289 (160 mg) was separated using chiral chromatography on a CHIRALPAK IF column with CO₂ and methanol as mobile phase to afford:

(R)-2-(3-(1,1,1-trifluoro-3-(4-methyl-4H-1,2,4-triazol-3-yl)propan-2-yl)phenyl)-4-(trifluoromethyl)isoindolin-1-one: (55 mg, off-white solid) ¹H NMR (500 MHz, DMSO-d₆) δ 8.22 (s, 1H), 8.06-8.00 (m, 2H), 7.98 (d, J=7.8 Hz, 1H), 7.81 (ddd, J=8.2, 2.2, 0.9 Hz, 1H), 7.73 (t, J=7.9 Hz, 1H), 7.37 (t, J=8.0 Hz, 1H), 7.24 (d, J=7.8 Hz, 1H), 5.14 (d, J=1.7 Hz, 2H), 4.30 (td, J=9.7, 5.2 Hz, 1H), 3.50 (s, 3H), 3.47-3.33 (m, 2H); LCMS: C₂₁H₁₆F₆N₄O requires: 454, found: m/z=455 [M+H]⁺.

(S)-2-(3-(1,1,1-trifluoro-3-(4-methyl-4H-1,2,4-triazol-3-yl)propan-2-yl)phenyl)-4-(trifluoromethyl)isoindolin-1-one: (15 mg, off-white solid)¹H NMR (500 MHz, DMSO-d₆) δ 8.22 (s, 1H), 8.06-7.98 (m, 2H), 7.98-7.95 (m, 1H), 7.81 (ddd, J=8.2, 2.3, 0.9 Hz, 1H), 7.77-7.70 (m, 1H), 7.37 (t, J=8.0 Hz, 1H), 7.24 (d, J=7.7 Hz, 1H), 5.14 (d, J=1.7 Hz, 2H), 4.30 (td, J=9.6, 5.2 Hz, 1H), 3.50 (s, 3H), 3.42-3.35 (m, 2H); LCMS: C₂₁H₁₆F₆N₄O requires: 454, found: m/z=455 [M+H]⁺.

### Example 472: N-{3-[(2R)-1-(4-methyl-4H-1,2,4-triazol-3-yl)propan-2-yl]phenyl}-6-(piperazin-1-yl)isoquinoline-3-carboxamide (472)

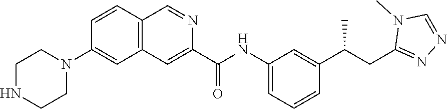 [SMILES: C[C@H](Cc1nncn1C)c1cccc(NC(=O)c2cc3cc(N4CCNCC4)ccc3cn2)c1]

Step 1: Synthesis of ethyl 6-(4-tert-butoxycarbonylpiperazin-1-yl)isoquinoline-3-carboxylate. To a stirring solution of ethyl 6-bromoisoquinoline-3-carboxylate (1.00 g, 3.57 mmol, 1.0 eq.) and palladium(II) acetate (80 mg, 0.36 mmol, 1.0 eq.) in dioxane (20 mL) was added potassium phosphate tribasic (2.35 g, 10.7 mmol, 3.0 eq.), 1-Boc-piperazine (0.82 g, 4.28 mmol, 1.2 eq.), and RuPhos (0.33 g, 0.71 mmol, 0.2 eq.). The reaction was heated to 120° C. for 4 h. The mixture was diluted with water and EtOAc. The phases were separated and the aqueous layer was extracted with EtOAc. Purification by flash column chromatography eluting with 0-40% EtOAc/hexanes gave the title compound (1.1 g, 2.85 mmol, 80%) as a colorless solid.

Step 2: Synthesis of tert-butyl 4-[3-({3-[(2R)-1-(4-methyl-1,2,4-triazol-3-yl)propan-2-yl]phenyl}carbamoyl)isoquinolin-6-yl]piperazine-1-carboxylate. A suspension of 3-[(2R)-1-(4-methyl-4H-1,2,4-triazol-3-yl)propan-2-yl]aniline (3.93 g, 18.1 mmol) in THF (100 mL) was cooled to 0° C. and treated with a solution of trimethylaluminum (2 M in toluene, 13.6 mL, 27.2 mmol) dropwise. After the addition was complete the solution was stirred at rt for 15 min. The mixture was added ethyl 6-{4-[(tert-butoxy)carbonyl]piperazin-1-yl}isoquinoline-3-carboxylate (3.50 g, 9.1 mmol) dropwise to the solution of in THF (5 mL). The solution was stirred at 40° C. for 16 h. The reaction was quenched with saturated aqueous Rochelle's salt and stirred for 1 h. The mixture was extracted twice with EtOAc, washed with brine, dried with sodium sulfate, filtered and evaporated to dryness. Purification by flash column chromatography eluting with 0-10% methanol in dichloromethane to afford the title compound (3.00 g, 60%).

Step 3: Synthesis of N-{3-[(2R)-1-(4-methyl-1,2,4-triazol-3-yl)propan-2-yl]phenyl}-6-(piperazin-1-yl)isoquinoline-3-carboxamide. To a mixture of THF (50 mL) and tert-butyl 4-[3-({3-[(2R)-1-(4-methyl-4H-1,2,4-triazol-3-yl)propan-2-yl]phenyl}carbamoyl)iso-quinolin-6-yl]piperazine-1-carboxylate (3.00 g, 5.40 mmol) was added hydrogen chloride (20 mL, 4 M in 1,4-dioxane). The mixture was allowed to stir at about 22° C. for 2 h. The volatiles were removed to afford the title compound (HCl salt, 2.20 g, 89%): ¹H NMR (500 MHz, DMSO-d₆) δ 10.58 (s, 1H), 9.13 (s, 1H), 8.41 (s, 1H), 8.29 (s, 1H), 8.06 (d, J=9.1 Hz, 1H), 7.89-7.76 (m, 2H), 7.63 (dd, J=9.2, 2.5 Hz, 1H), 7.36 (d, J=2.4 Hz, 1H), 7.29 (t, J=7.8 Hz, 1H), 7.01 (d, J=7.7 Hz, 1H), 3.46 (s, 3H), 3.38-3.33 (m, 4H), 3.32 (s, 3H), 3.26 (q, J=7.1 Hz, 1H), 2.99 (d, J=7.4 Hz, 2H), 2.91-2.85 (m, 3H), 1.30 (d, J=7.0 Hz, 3H), 1.04 (d, J=6.1 Hz, 1H); LCMS: C₂₆H₂₉N₇O requires: 455, found: m/z=456 [M+H]⁺.

### Example 473, 474a and 474b: 2-(3-((1R,2S)-1-fluoro-1-(4-methyl-4H-1,2,4-triazol-3-yl)propan-2-yl)phenyl)-4-(trifluoromethyl)isoindolin-1-one (racemic, 473) and 2-(3-((1S, 2S)-1-fluoro-1-(4-methyl-4H-1,2,4-triazol-3-yl)propan-2-yl)phenyl)-4-(trifluoromethyl)isoindolin-1-one (474)

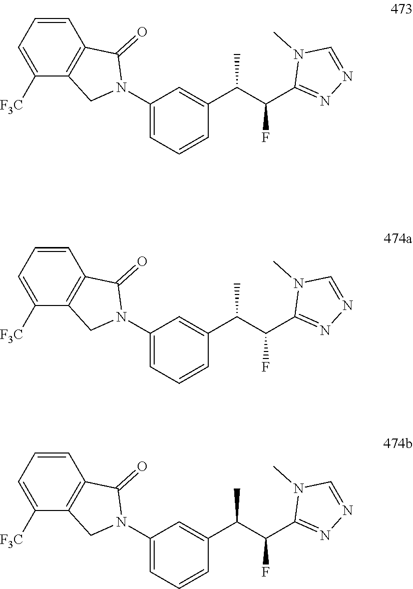 [SMILES: C[C@@H](c1cccc(N2Cc3c(cccc3C(F)(F)F)C2=O)c1)[C@H](F)c1nncn1C ; C[C@@H](c1cccc(N2Cc3c(cccc3C(F)(F)F)C2=O)c1)[C@@H](F)c1nncn1C ; C[C@H](c1cccc(N2Cc3c(cccc3C(F)(F)F)C2=O)c1)[C@H](F)c1nncn1C]

Step 1: Synthesis of (2S,3S and 2R,3R)-ethyl 2-hydroxy-3-(3-nitrophenyl)butanoate. To a solution of ethyl 3-(3-nitrophenyl)-2-oxobutanoate (2.0 g, 7.97 mmol) in THF (50 mL) was added L-selectride (9.6 mL, 9.60 mmol, 1 M in THF) dropwise at −78° C. under N₂. The mixture was stirred at −78° C. for 1.5 h under N₂. The mixture was quenched by the addition of saturated NH₄Cl (aq. 100 mL). The mixture was extracted with EtOAc. The combined organic layers were washed by brine, dried, and concentrated to afford (2S,3S and 2R, 3R)-ethyl 2-hydroxy-3-(3-nitrophenyl)butanoate (1.9 g, crude) as a yellow oil, which was used without purification. MS (ESI) calculated for (C₁₂H₁₅NO₅) [M+H]⁺, 254.2; found, 254.2.

Step 2: Synthesis of (2S,3S and 2R,3R)-2-hydroxy-3-(3-nitrophenyl)butanoic acid. To a solution of (2S,3S and 2R,3R)-ethyl 2-hydroxy-3-(3-nitrophenyl)butanoate (1.9 g, 7.51 mmol) in THF (20 mL) and water (20 mL) was added LiOH (361.5 mg, 15.10 mmol) at 0° C. The mixture was stirred at 25° C. for 2 h. The pH value was adjusted to 3 by HCl (2 N). The solids were collected by filtration and dried to afford (2S,3S and 2R 3R)-2-hydroxy-3-(3-nitrophenyl)butanoic acid (1.5 g, crude) as a colorless solid, which was used without purification.

Step 3: Synthesis of 2-((2S,3S and 2R,3R)-2-hydroxy-3-(3-nitrophenyl)butanoyl)-N-methylhydrazinecarbo thioamide. To a mixture of (2S,3S and 2R, 3R)-2-hydroxy-3-(3-nitrophenyl)butanoic acid (1.5 g, 6.67 mmol), N-(3-dimethylaminopropyl)-N′-ethylcarbodimide hydrochloride (1.4 g, 7.34 mmol), N-methylhydrazinecarbothioamide (0.8 g, 8.00 mmol) and HOBt (1.2 g, 8.67 mmol) in DMF (20 mL) was added triethylamine (6.7 g, 66.70 mmol) at 0° C. The mixture was stirred at 25° C. for 16 h. The mixture was diluted with water and then filtered. The solids were collected and dried to afford the title compound (1.1 g, crude) as a colorless solid, which was used without purification. MS (ESI) calculated for (Cl₂H₁₆N₄O₄S) [M+H]⁺, 313.1; found, 313.2.

Step 4: Synthesis of 5-((1S,2S and 1R,2R)-1-hydroxy-2-(3-nitrophenyl)propyl)-4-methyl-2H-1,2,4-triazole-3 (4H)-thione. To a solution of 2-((2S,3S and 2R,3R)-2-hydroxy-3-(3-nitrophenyl)butanoyl)-N-methylhydrazinecarbothioamide (1.1 g, 3.52 mmol) in ethanol (17 mL) was added NaOH (2M, 17 mL, 34.00 mmol) at 25° C. The mixture was stirred at 100° C. for 1 h. The pH value of the mixture was adjusted to 3 by HCl (2 N). The solids were collected by filtration and dried to afford the title compound (4H)-thione (800 mg, crude) as a colorless solid. MS (ESI) calculated for (C₁₂H₁₄N₄O₃S) [M+H]⁺, 295.1; found, 295.2.

Step 5: Synthesis of (1S,2S and 1R,2R)-1-(4-methyl-4H-1,2,4-triazol-3-yl)-2-(3-nitrophenyl)propan-1-ol. To a mixture of 5-((1S,2S and 1R, 2R)-1-hydroxy-2-(3-nitrophenyl)propyl)-4-methyl-2H-1,2,4-triazole-3 (4H)-thione (800 mg, 2.72 mmol) and NaNO₂ (1.9 g, 27.20 mmol) was added HNO₃ (aq. 1 M, 27 mL, 27 mmol) at 0° C. The mixture was stirred at 25° C. for 90 minutes. The reaction was then quenched by the addition of saturated NaHCO₃ solution and extracted by methylene chloride, washed with brine. The combined organic layers were dried, dried, and filtered. The filtrated was concentrated. The residue was purified by flash column chromatography with 07% of methanol in dichloromethane to afford the title compound (500 mg, 25% over 5 steps) as a yellow oil. MS (ESI) calculated for (C₁₂H₁₄N₄O₃) [M+H]⁺, 263.1; found, 263.2.

Step 6: Synthesis of (1S,2S and 1R,2R)-2-(3-aminophenyl)-1-(4-methyl-4H-1,2,4-triazol-3-yl)propan-1-ol. To a solution of ((1S, 2S and 1R,2R)-1-(4-methyl-4H-1,2,4-triazol-3-yl)-2-(3-nitrophenyl)propan-1-ol (500 mg, 1.91 mmol) in methanol (20 mL) was added anhydrous Pd/C (100 mg, 10%). The mixture was stirred at 25° C. for 2 h under H₂. The mixture was filtered. The filtrate was concentrated to the title compound (400 mg, crude) as a colorless oil. MS (ESI) calculated for (C₁₂H₁₆N₄O) [M+H]⁺, 233.2; found, 233.2.

Step 7: Synthesis of 2-(3-((1S,2S and 1R,2R)-1-hydroxy-1-(4-methyl-4H-1,2,4-triazol-3-yl)propan-2-yl)phenyl)-4-(trifluoromethyl)isoindolin-1-one. The indolone formation reaction was carried out in a manner similar to 260, step 2 using (1S,2S and 1R,2R)-2-(3-aminophenyl)-1-(4-methyl-4H-1,2,4-triazol-3-yl)propan-1-ol (800 mg, 3.44 mmol) and methyl 2-(bromomethyl)-3-(trifluoromethyl)benzoate (1.22 g, 4.14 mmol) in methanol (10 mL) to afford the title compound (400 mg, 28%) as a colorless solid. MS (ESI) calculated for (C₂₁H₁₉F₃N₄O₂) [M+H]⁺, 417.1; found, 417.1.

Step 8: Synthesis of 2-(3-((1R,2S)-1-fluoro-1-(4-methyl-4H-1,2,4-triazol-3-yl)propan-2-yl)phenyl)-4-(trifluoromethyl)isoindolin-1-one (474a) and 2-(3-((1S,2R)-1-fluoro-1-(4-methyl-4H-1,2,4-triazol-3-yl)propan-2-yl)phenyl)-4-(trifluoromethyl)isoindolin-1-one (474b) and 2-(3-((1S,2S and 7R,2R)-1-fluoro-1-(4-methyl-4H-1,2,4-triazol-3-yl)propan-2-yl)phenyl)-4-(trifluoromethyl)isoindolin-1-one (racemic 473). To a solution of racemic 2-(3-((1S,2S)-1-hydroxy-1-(4-methyl-4H-1,2,4-triazol-3-yl)propan-2-yl)phenyl)-4-(trifluoromethyl)isoindolin-1-one (400.0 mg, 0.96 mmol) in dichloromethane (20 mL) was added DAST (1.5 g, 9.6 mmol) at 0° C. The solution was stirred at 25° C. for 16 h. The reaction was then quenched by the addition of saturated sodium bicarbonate aqueous (50 mL), and extracted with dichloromethane (80 mL×3). The organic layers were combined, washed with brine, dried, and concentrated. The crude product was purified by Prep-HPLC to afford a mixture of 2-(3-(l-fluoro-1-(4-methyl-4H-1,2,4-triazol-3-yl)propan-2-yl)phenyl)-4-(trifluoromethyl)isoindolin-1-one (150 mg, 30%) as a colorless solid. The mixture was then separated by Chiral-Prep-HPLC with following conditions: [Column: CHIRAL ART Cellulose-SB, 2*25 cm,5 um; Mobile Phase A: Hex (8 mmol/L NH₃.methanol); Mobile Phase B: EtOH; Flow rate: 20 mL/min; Gradient: 30% B to 30% B in 18 min; 254/220 nm] to afford:

2-(3-((1R,2S)-1-fluoro-1-(4-methyl-4H-1,2,4-triazol-3-yl)propan-2-yl)phenyl)-4-(trifluoromethyl)isoindolin-1-one (474a): (first peak, 41 mg, colorless solid) MS (ESI) calculated for (C₂₁H₁₉F₄N₄O) [M+H]⁺, 419.1; found, 418.8. ¹H NMR (400 MHz, DMSO-d₆) δ 8.40 (s, 1H), 8.09-8.03 (m, 2H), 7.82-7.78 (m, 3H), 7.36-7.32 (m, 1H), 7.12 (d, J=7.6 Hz, 1H), 6.09-5.96 (m, 1H), 5.17 (s, 2H), 3.83-3.76 (m, 1H), 3.58 (s, 3H), 1.53-1.51 (m, 3H). ¹⁹F NMR (300 MHz, DMSO) δ-60.01, −180.58.

2-(3-((1S,2R)-1-fluoro-1-(4-methyl-4H-1,2,4-triazol-3-yl)propan-2-yl)phenyl)-4-(trifluoromethyl)isoindolin-1-one (474b): (second peak, 55 mg, colorless solid) MS (ESI) calculated for (C₂₁H₁₉F₄N₄O) [M+H]⁺, 419.1; found, 418.9. ¹H NMR (400 MHz, DMSO-d₆) δ 8.40 (s, 1H), 8.09-8.03 (m, 2H), 7.82-7.78 (m, 3H), 7.36-7.32 (m, 1H), 7.12 (d, J=7.6 Hz, 1H), 6.09-5.96 (m, 1H), 5.17 (s, 2H), 3.83-3.76 (m, 1H), 3.58 (s, 3H), 1.53 (d, J=6.1 Hz, 3H). ¹⁹F NMR (300 MHz, DMSO) δ-60.50, −180.58.

2-(3-((1S,2S and 1R,2R)-1-fluoro-1-(4-methyl-4H-1,2,4-triazol-3-yl)propan-2-yl)phenyl)-4-(trifluoromethyl)isoindolin-1-one (racemic 473): (third peak, 8.1 mg, colorless solid) MS (ESI) calculated for (C₂₁H₁₉F₄O) [M+H]+, 419.1; found, 418.8. ¹H NMR (400 MHz, DMSO-d₆) δ 8.55 (s, 1H), 8.10 (d, J=7.6 Hz, 1H), 8.05 (d, J=7.6 Hz, 1H), 7.95 (s, 1H), 7.90-7.88 (m, 1H), 7.81 (t, J=7.6 Hz, 1H), 7.45 (t, J=8.0 Hz, 1H), 7.30 (d, J=8.0 Hz, 1H), 6.09-5.95 (m, 1H), 5.25 (s, 2H), 3.80-3.77 (m, 1H), 3.71 (s, 3H), 1.22 (d, J=7.2 Hz, 3H). ¹⁹F NMR (300 MHz, DMSO) δ-59.98, −173.65.

### Example 475: (S)-4-(3-hydroxypyrrolidine-1-carbonyl)-N-(3-(2-methyl-1-(4-methyl-4H-1,2,4-triazol-3-yl)propan-2-yl)phenyl)-6-(trifluoromethyl)picolinamide (475)

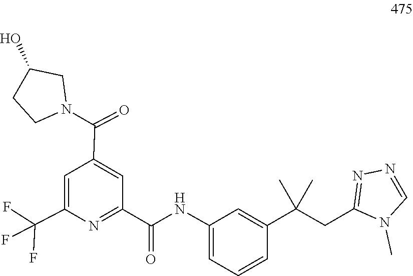 [SMILES: Cn1cnnc1CC(C)(C)c1cccc(NC(=O)c2cc(C(=O)N3CC[C@H](O)C3)cc(C(F)(F)F)n2)c1]

Step 1: Synthesis of 2-chloro-6-(trifluoromethyl)isonicotinic acid. A mixture of 2-chloro-6-(trifluoromethyl)isonicotinonitrile (15.0 g, 72.62 mmol) in concentrated HCl (150 mL) was stirred at 70° C. for 5 h. The reaction was diluted with water and the solids were collected by filtration to afford 2-chloro-6-(trifluoromethyl)isonicotinic acid (10 g, crude) as a white solid, which was used in the next step without purification. MS (ESI) calculated for (C₇H₃ClF₃NO₂) [M−H]⁻, 224.0; found, 223.8.

Step 2: Synthesis of (S)-(2-chloro-6-(trifluoromethyl)pyridin-4-yl)(3-hydroxypyrrolidin-1-yl)methanone. To a solution of 2-chloro-6-(trifluoromethyl)isonicotinic acid (3.8 g, 16.85 mmol) in DMF (50 mF) were added (S)-pyrrolidin-3-ol hydrochloride (3.1 g, 25.30 mmol), DIEA (10.8 g, 83.91 mmol) and HATU (12.8 g, 33.61 mmol). The solution was stirred at rt for 3 h. The reaction was diluted with water and the aqueous phase was extracted with EtOAc. The combined organic layers were washed with brine, dried, and filtered. The filtrate was evaporated under vacuum. The residue was purified by reverse phase flash column chromatography with 5-100% CH₃CN in water to the title compound (2.5 g, 50%) as a yellow solid. MS (ESI) calculated for (C₁₁H₁₀ClF₃N₂O₂) [M+H]⁺, 295.0; found, 294.8.

Step 3: Synthesis of (S)-4-(3-hydroxypyrrolidine-1-carbonyl)-6-(trifluoromethyl)picolinic acid. To a solution of (S)-(2-chloro-6-(trifluoromethyl)pyridin-4-yl)(3-hydroxypyrrolidin-1-yl)methanone (2.0 g, 6.79 mmol) in dioxane/water (20/5 mL) were added Pd(OAc)₂ (153.1 mg, 0.68 mmol), XantPhos (786.4 mg, 1.36 mmol) and TEA (6.9 g, 67.90 mmol). The solution was stirred at 90° C. for 16 h under CO (20 atm). The reaction was diluted with water and the aqueous phase was extracted with EtOAc. The combined organic layers were combined, washed with brine, dried, and filtered. The filtrate was evaporated under vacuum. The residue was purified by reverse phase flash column chromatography with 5-35% acetonitrile in water to the title compound (974 mg, 47%) as an off-white solid. MS (ESI) calculated for (C₁₂H₁₁F₃N₂O₄) [M+H]⁺, 305.1; found, 304.8.

Step 4: Synthesis of (S)-4-(3-hydroxypyrrolidine-1-carbonyl)-N-(3-(2-methyl-1-(4-methyl-4H-1,2,4-triazol-3-yl)propan-2-yl)phenyl)-6-(trifluoromethyl)picolinamide (475). To a solution of (S)-4-(3-hydroxypyrrolidine-1-carbonyl)-6-(trifluoromethyl)picolinic acid (100.0 mg, 0.33 mmol) in DMF (5 mL) were added 3-(2-methyl-1-(4-methyl-4H-1,2,4-triazol-3-yl)propan-2-yl)aniline (75.7 mg, 0.33 mmol), DIEA (212.2 mg, 1.64 mmol) and HATU (250.0 g, 0.66 mmol). The solution was stirred at rt for 3 h. The reaction was diluted with water and the aqueous phase was extracted with EtOAc. The combined organic layers were washed with brine, dried, and filtered. The filtrate was evaporated under vacuum. The residue was purified by Prep-HPLC to afford the title compound (29.3 mg, 17%) as an off-white solid. MS (ESI) calculated for (C₂₅H₂₇F₃N₆O₃) [M+H]⁺, 517.2; found, 516.9. H NMR (300 MHz, DMSO-d₆) δ 10.42 (s, 1H), 8.38 (d, J=2.4 Hz, 1H), 8.25-8.22 (m, 2H), 7.79-7.73 (m, 2H), 7.32-7.27 (m, 1H), 7.09 (d, J=7.8 Hz, 1H), 5.10-5.03 (m, 1H), 4.41-4.21 (m, 1H), 3.63-3.42 (m, 4H), 3.25 (s, 3H), 3.08-2.97 (m, 2H), 2.01-1.83 (m, 2H), 1.43 (s, 6H).

### Example 476: 2-(3-cyclobutyl-5-(1-(4-methyl-4H-1,2,4-triazol-3-yl)propan-2-yl)phenyl)-4-(trifluoromethyl)isoindolin-1-one (476)

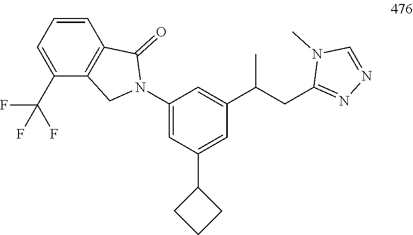 [SMILES: CC(Cc1nncn1C)c1cc(C2CCC2)cc(N2Cc3c(cccc3C(F)(F)F)C2=O)c1]

Step 1: Synthesis of ethyl (E)-3-(3,5-dibromophenyl)but-2-enoate. To a stirring solution of sodium hydride (4.32 g, 108 mmol, 2.0 eq.) in THF (360 mL) at 0° C., was added triethyl phosphonoacetate (24.5 mL, 118.7 mmol, 2.2 eq.). The reaction was allowed to proceed for 30 min at rt. This solution was added to a stirring solution of l-(3,5-dibromophenyl)ethanone (15 g, 54.0 mmol, 1.0 eq.) in THF (360 mL). The reaction was allowed to proceed for 12 hours. The mixture was concentrated then purified by flash column chromatography eluting with 0-30% EtOAc/hexanes to give the title compound (14.2 g, 40.8 mmol, 76% yield) as a colorless solid.

Step 2: Synthesis of ethyl (E)-3-[3-bromo-5-[1-oxo-4-(trifluoromethyl)isoindolin-2-yl]phenyl]but-2-enoate. To a stirring solution of ethyl (E)-3-[3-bromo-5-[1-oxo-4-(trifluoromethyl)isoindolin-2-yl]phenyl]but-2-enoate (10.1 g, 28.7 mmol, 1.0 eq.), 4-(trifluoromethyl)isoindolin-1-one (5.8 g, 28.7 mmol, 1.0 eq.), and cesium carbonate (19.0 g, 57.47 mmol, 2.0 eq.) in dioxane (290 mL) was added palladium(ii) acetate (0.65 g, 2.87 mmol, 0.1 eq.) and 4,5-bis(diphenylphosphino)-9,9-dimethylxanthene (3.43 g, 5.75 mmol, 0.2 eq.). The reaction was allowed to proceed for 14 h at 120° C. The mixture was added to a separatory funnel that contained a mixture of a 1 M HCl and EtOAc. The phases were separated and the aqueous layer was extracted with EtOAc. Purification by flash column chromatography eluting with 0-40% EtOAc/hexanes to give ethyl the title compound (6.5 g, 13.88 mmol, 48% yield) as a colorless solid.

Step 3: Synthesis of ethyl 3-[3-bromo-5-[1-oxo-4-(trifluoromethyl)isoindolin-2-yl]phenyl]butanoate. To a solution of ethyl (E)-3-[3-bromo-5-[1-oxo-4-(trifluoromethyl)isoindolin-2-yl]phenyl]but-2-enoate (4.5 g, 9.61 mmol, 1.0 eq.) in EtOAc (250 mL) under nitrogen was added platinum(IV) oxide (218 mg, 0.96 mmol, 0.1 eq.). The reaction was put under a hydrogen balloon and stirred for 5 days. The suspension was filtered through Celite and concentrated. The crude product was used on the next step.

Step 4: Synthesis of 3-[3-bromo-5-[1-oxo-4-(trifluoromethyl)isoindolin-2-yl]phenyl]butanoic acid. To a stirring solution of ethyl 3-[3-bromo-5-[1-oxo-4-(trifluoromethyl)isoindolin-2-yl]phenyl]butanoate (3.4 g, 7.23 mmol, 1.0 eq.) in ethanol (70 mL) and water (10 mL) was added lithium hydroxide (1.7 g, 72.3 mmol, 10 eq.). The mixture was stirred at 80° C. for 12 h. The reaction was concentrated, diluted water, and extracted with EtOAc. The organic layer was dried with sodium sulfate, filtered, and concentrated under reduced pressure. The crude product was used without purification.

Step 5: Synthesis of l-[3-[3-bromo-5-[1-oxo-4-(trifluoromethyl)isoindolin-2-yl]phenyl]butanoylamino]-3-methyl-thiourea. HOBT (1.32 g, 8.66 mmol, 1.2 eq.) was added to a mixture of 3-[3-bromo-5-[1-oxo-4-(trifluoromethyl)isoindolin-2-yl]phenyl]butanoic acid (3.19 g, 7.21 mmol, 1 eq.), N-methylhydrazinecarbothioamide (1.85 g, 7.21 mmol, 1 eq.), EDC-HCl (1.66 g, 8.66 mmol, 1.2 eq.), and 4-methylmorpholine (4.0 mL, 36.1 mmol, 5 eq.) in dimethylformamide (36 mL). The mixture was allowed to stir at rt for 1h. EtOAc and water were added. The organic layer was dried, filtered, concentrated and carried into the next step.

Step 6: Synthesis of 2-[3-bromo-5-[1-methyl-2-(4-methyl-5-sulfanyl-1,2,4-triazol-3-yl)ethyl]phenyl]-4-(trifluoromethyl)isoindolin-1-one. To a stirring solution of 1-[3-[3-bromo-5-[1-oxo-4-(trifluoromethyl)isoindolin-2-yl]phenyl]butanoylamino]-3-methyl-thiourea (3.81 g, 7.2 mmol, 1.0 eq.) in THE (36 mL) was added KOH (10 mL, 2M) and the mixture was stirred for 2 h. The solution was added to a separatory funnel and extracted with EtOAc. The combined organic layers were dried, filtered, and concentrate. The crude material was used without purification.

Step 7: Synthesis of 2-[3-bromo-5-[1-methyl-2-(4-methyl-1,2,4-triazol-3-yl)ethyl]phenyl]-4-(trifluoromethyl)isoindolin-1-one. To a stirring solution of 2-[3-bromo-5-[1-methyl-2-(4-methyl-5-sulfanyl-1,2,4-triazol-3-yl)ethyl]phenyl]-4-(trifluoromethyl)isoindolin-1-one (3.68 g, 7.2 mmol, 1.0 eq.) in dichloromethane (20 mL) and acetic acid (20 mL) was added hydrogen peroxide (7.35 mL, 71.96 mmol, 10 eq.). The mixture was stirred at rt for about 14 h. The mixture was concentrated then purified by flash column chromatography eluting with 0-10% methanol/methylene chloride to give the title compound (1.56 g, 3.25 mmol, 45% yield) as a colorless solid.

Step 8: Synthesis of 2-(3-cyclobutyl-5-(1-(4-methyl-4H-1,2,4-triazol-3-yl)propan-2-yl)phenyl)-4-(trifluoromethyl)isoindolin-1-one. To a solution of 2-[3-bromo-5-[1-methyl-2-(4-methyl-1,2,4-triazol-3-yl)ethyl]phenyl]-4-(trifluoromethyl)isoindolin-1-one (100 mg, 0.21 mmol, 1.0 eq.), tetrakis(triphenylphosphine) palladium(O) (20 mg, 0.02 mmol, 0.1 eq.), in THF (2 mL) under nitrogen was added bromo(cyclobutyl)zinc (1.43 mL, 0.71 mmol, 3.4 eq.). The reaction was allowed to proceed for 4 h at 100° C. The reaction was quenched with saturated aqueous ammonium chloride and extracted with EtOAc. The combined organic layers were washed with brine and dried, filtered. The residue was purified by flash column chromatography eluting with 0-10% methanol in dichloromethane to afford the title compound (16 mg, 0.04 mmol, 17% yield): ¹H NMR (500 MHz, DMSO-d6) δ 8.28 (s, 1H), 8.08 (d, J=7.6 Hz, 1H), 8.05 (d, J=7.7 Hz, 1H), 7.81 (t, J=7.7 Hz, 1H), 7.68 (d, J=2.0 Hz, 1H), 7.61 (t, J=1.8 Hz, 1H), 5.22 (s, 2H), 3.53 (p, J=8.7 Hz, 1H), 3.43 (s, 2H), 3.31-3.25 (m, 1H), 3.07-2.91 (m, 2H), 2.30 (dtt, J=12.1, 8.0, 2.5 Hz, 2H), 2.17-2.05 (m, 2H), 1.99 (qt, J=9.9, 8.1 Hz, 2H), 1.88-1.80 (m, 2H), 1.31 (d, J=6.9 Hz, 3H); LCMS: requires: 454, found: m/z=455 [M+H]⁺.

### Example 477: 6-cyclopropyl-2-(3-(2-methyl-1-(4-methyl-4H-1,2,4-triazol-3-yl)propan-2-yl)phenyl)-4-(trifluoromethyl)isoindolin-1-one (477)

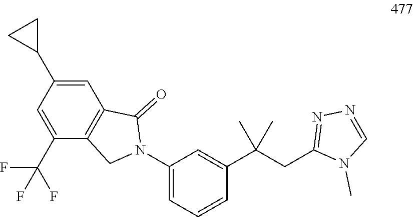 [SMILES: Cn1cnnc1CC(C)(C)c1cccc(N2Cc3c(cc(C4CC4)cc3C(F)(F)F)C2=O)c1]

Step 1: Synthesis of 6-cyclopropyl-4-(trifluoromethyl)isoindolin-1-one. To a solution of 6-bromo-4-(trifluoromethyl)isoindolin-1-one (500.0 mg, 1.79 mmol) in toluene/water(10/1 mL) were added potassium cyclopropyltrifluoroboranuide (528.6 mg, 3.57 mmol), Pd(dppf)Cl₂(130.5 mg, 0.18 mmol) and K₃PO₄ (1.1 g, 5.35 mmol). The solution was stirred at 100° C. for 4 h under nitrogen. The reaction was diluted with water and the aqueous phase was extracted with EtOAc. The combined organic layers were washed with brine, dried, and filtered. The filtrate was evaporated under vacuum. The residue was purified by flash column chromatography with 0˜10% methanol in methylene chloride to afford the title compound (300.0 mg, 70%) as a yellow solid. MS (ESI) calculated for (C₁₂H₁₀F₃NO) [M+H]⁺, 242.1; found, 241.9.

Step 2: Synthesis of 6-cyclopropyl-2-(3-(2-methyl-1-(4-methyl-4H-1,2,4-triazol-3-yl)propan-2-yl)phenyl)-4-(trifluoromethyl)isoindolin-1-one (477). A degassed solution of 6-cyclopropyl-4-(trifluoromethyl)isoindolin-1-one (100.0 mg, 0.41 mmol), 3-(2-(3-bromophenyl)-2-methylpropyl)-4-methyl-4H-1,2,4-triazole (183.0 mg, 0.62 mmol), Pd(OAc)₂ (28.0 mg, 0.12 mmol), XantPhos (143.9 mg, 0.25 mmol), Cs₂CO₃ (405.8 mg, 1.25 mmol) in dioxane (5 mL) was stirred at 100° C. for 16 h under nitrogen. The reaction was quenched by the addition of water, and the aqueous phase was extracted with EtOAc. The combined organic layers were washed with brine, dried, and filtered. The filtrate was evaporated under vacuum. The residue was purified by Prep-HPLC to afford the title compound (9.5 mg, 5%) as a colorless solid. MS (ESI) calculated for (C₂₅H₂₅F₃N₄O) [M+H]⁺, 455.2; found, 455.1. ¹H NMR (300 MHz, DMSO-d₆) δ 8.22 (s, 1H), 7.96-7.58 (m, 4H), 7.33 (d, J=7.5 Hz, 1H), 7.13 (d, J=6.3 Hz, 1H), 5.11 (s, 2H), 3.14 (s, 3H), 3.01 (s, 2H), 2.24-2.21 (m, 1H), 1.46 (s, 6H), 1.09-1.06 (m, 2H), 0.89-0.84 (m, 2H).

### Examples 478a, 478b, 479, 480: 2-(3-((1S,2R)-1,2-difluoro-1-(4-methyl-4H-1,2,4-triazol-3-yl)propan-2-yl)phenyl)-4-(trifluoromethyl)isoindolin-1-one (478a) and 2-(3-((1R,2S)-1,2-difluoro-1-(4-methyl-4H-1,2,4-triazol-3-yl)propan-2-yl)phenyl)-4-(trifluoromethyl)isoindolin-1-one (478b)

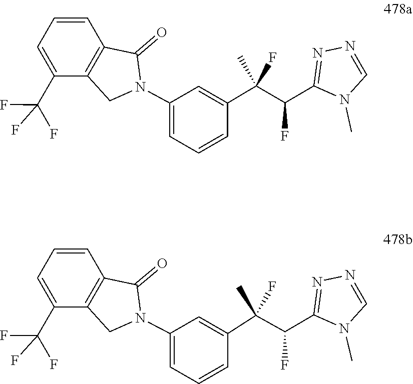 [SMILES: Cn1cnnc1[C@@H](F)[C@](C)(F)c1cccc(N2Cc3c(cccc3C(F)(F)F)C2=O)c1 ; Cn1cnnc1[C@H](F)[C@@](C)(F)c1cccc(N2Cc3c(cccc3C(F)(F)F)C2=O)c1]

### 2-(3-((1S,2S)-1,2-difluoro-1-(4-methyl-4H-1,2,4-triazol-3-yl)propan-2-yl)phenyl)-4-(trifluoromethyl)isoindolin-1-one (479) and 2-(3-((1R,2R)-1,2-difluoro-1-(4-methyl-4H-1,2,4-triazol-3-yl)propan-2-yl)phenyl)-4-(trifluoromethyl)isoindolin-1-one (480)

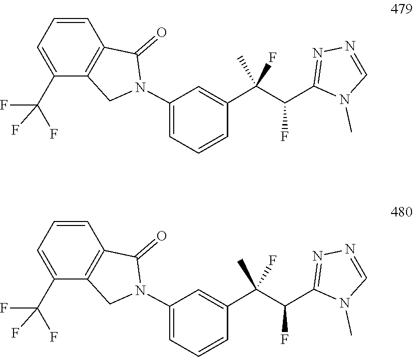 [SMILES: Cn1cnnc1[C@H](F)[C@](C)(F)c1cccc(N2Cc3c(cccc3C(F)(F)F)C2=O)c1 ; Cn1cnnc1[C@@H](F)[C@@](C)(F)c1cccc(N2Cc3c(cccc3C(F)(F)F)C2=O)c1]

Step 1: Synthesis of ethyl 2,3-dihydroxy-3-(3-nitrophenyl)butanoate. To a mixture of ethyl (2E)-3-(3-nitrophenyl)but-2-enoate (25.0 g, 106.27 mmol), NMO (13.7 g, 116.90 mmol), citric acid (5.1 g, 26.57 mmol) in tBuOH (15 mL) and water (15 mL) was added tetraoxoosmium (27.0 mg, 0.11 mmol). The solution was stirred at 25° C. for 16 h. The reaction was then quenched by the addition of 750 mL of HCl (1N) and the aqueous phase was extracted with EtOAc (500 mL×3). The combined organic layers were washed with brine, dried, filtered and concentrated. The residue was purified by flash column chromatography with 10-55% EtOAc in petroleum ether to afford the title compound (30 g, ˜80% purity) as a brown syrup, which was used without purification. MS (ESI) calculated for (C₁₂H₁₅NO₆) [M+H]⁺, 270.0; found, 270.0.

Step 2: Synthesis of 2,3-dihydroxy-3-(3-nitrophenyl)butanehydrazide. To a solution of ethyl 2,3-dihydroxy-3-(3-nitrophenyl)butanoate (60.0 g, 222.8 mmol) in methanol (600 mL) was added hydrazine hydrate (130.0 g) at 0° C. The solution was stirred at 25° C. for 16 h. The mixture was concentrated to afford the title compound (60.0 g, crude) as a yellow solid, which was used without purification. MS (ESI) calculated for (C₁₀H₁₃N₃O₅) [M+H]⁺, 256.2; found, 256.1.

Step 3: Synthesis of 2-(2,3-dihydroxy-3-(3-nitrophenyl)butanoyl)-N-methylhydrazine-1-carbothioamide. To a solution of 2,3-dihydroxy-3-(3-nitrophenyl)butanehydrazide (33.0 g, 129.3 mmol) in methanol (350.0 mL) was added isothiocyanatomethane (16.0 g, 218.9 mmol). The solution was stirred at 80° C. for 19 h. When the reaction was completed, the solids were filtered out. The filtrate was concentrated to afford residue, which was purified by trituration with methanol/dichloromethane (1/10) three times to afford the title compound (31.0 g, crude) as a colorless solid, which was used without purification. MS (ESI) calculated for (C₁₂H₁₆N₄O₅S) [M+H]⁺, 329.3; found, 329.0.

Step 4: Synthesis of 1-(5-mercapto-4-methyl-4H-1,2,4-triazol-3-yl)-2-(3-nitrophenyl)propane-1,2-diol. A mixture of 2,3-dihydroxy-N-[(methylcarbamothioyl)amino]-3-(3-nitrophenyl)butanamide (31.0 g, crude) in NaOH aqueous (950 mL, 1 N) was stirred at 25° C. for 48 h. When the reaction was completed, the pH value of the mixture was adjusted to 6 by HCl (6 N). The solids were collected by filtration and dried to afford the title compound (20.0 g, crude) as a yellow solid, which was used without purification. MS (ESI) calculated for (C₁₂H₁₄N₄O₄S) [M+H]⁺, 311.1; found, 311.0.

Step 5: Synthesis of l-(4-methyl-4H-1,2,4-triazol-3-yl)-2-(3-nitrophenyl)propane-1,2-diol. To a mixture of l-(4-methyl-5-sulfanyl-4H-1,2,4-triazol-3-yl)-2-(3-nitrophenyl)propane-1,2-diol (10.0 g, 32.22 mmol) in acetic acid (40 mL) and methylene chloride (120 mL) was added hydrogen peroxide (13.4 g, 118.19 mmol) at 0° C. The solution was stirred at 25° C. for 1.5 h. When the reaction was completed, the pH value of the aqueous phase was adjusted to 9 with NaOH (4 N). The solids were collected by filtration and dried to afford the title compound (10.4 g, crude) as a yellow solid, which was used without purification. MS (ESI) calculated for (C₁₂H₁₄N₄O₄) [M+H]⁺, 279.2; found, 279.0.

Step 6: Synthesis of 3-(1,2-difluoro-2-(3-nitrophenyl)propyl)-4-methyl-4H-1,2,4-triazole. To a solution of l-(4-methyl-4H-1,2,4-triazol-3-yl)-2-(3-nitrophenyl)propane-1,2-diol (7.0 g, 25.16 mmol) in methylene chloride (120 mL) was added DAST (21.0 g, 130.28 mmol) dropwise at −40° C. The solution was stirred below 0° C. for 3 h. The mixture was then poured into NaHCO₃ (sat. aq. 1600 mL) at 0° C., and then extracted with dichloromethane (500 mL×3). The combined organic layer was washed with brine, dried, filtered and concentrated. The residue was purified by flash column chromatography with 0-10% methanol in dichloromethane to afford 3-[1,2-difluoro-2-(3-nitrophenyl)propyl]-4-methyl-4H-1,2,4-triazole (6.0 g, 84%) as a yellow solid. MS (ESI) calculated for (C₁₂H₁₂F₂N₄O₂) [M+H]⁺, 283.2; found, 283.0.

Step 7: Synthesis of 3-(1,2-difluoro-1-(4-methyl-4H-1,2,4-triazol-3-yl)propan-2-yl)aniline. To a solution of 3-[1,2-difluoro-2-(3-nitrophenyl)propyl]-4-methyl-4H-1,2,4-triazole (3.4 g, 12.05 mmol) in methanol (100 mL) was added Pd/C (dry, 1.0 g) which was stirred at 25° C. for 18 h under H₂ (2 atm). The solids were filtered out. The filtrate was concentrated to afford 3-[1,2-difluoro-1-(4-methyl-4H-1,2,4-triazol-3-yl)propan-2-yl]anilin (2.6 g, crude) as a colorless syrup, which was used without purification. MS (ESI) calculated for (C₁₂H₁₄F₂N₄) [M+H]⁺, 253.2; found, 253.0.

Step 9 & 10: Synthesis of racemic 2-(3-((1R,2R)-1,2-difluoro-1-(4-methyl-4H-1,2,4-triazol-3-yl)propan-2-yl)phenyl)-4-(trifluoromethyl)isoindolin-1-one and racemic 2-[3-[(1S,2R)-1,2-difluoro-1-(4-methyl-4H-1,2,4-triazol-3-yl)propan-2-yl]phenyl]-4-(trifluoromethyl)-2,3-dihydro-1H-isoindol-1-one. A mixture of 3-[1,2-difluoro-1-(4-methyl-4H-1,2,4-triazol-3-yl)propan-2-yl]aniline (990 mg, crude), methyl 2-(bromomethyl)-3-(trifluoromethyl)benzoate (700 mg, 2.36 mmol) and AgNO₃ (570 mg, 3.37 mmol) in methanol (30 mL) was stirred at 25° C. for 16 h. The solids were filtered out. The filtrate was concentrated to give the residue, which was purified by flash column chromatography with 50-65% acetonitrile in water to afford the mixture containing four isomers. The mixture was then separated by Prep-HPLC to afford:

Racemic 2-(3-((1R,2R)-1,2-difluoro-1-(4-methyl-4H-1,2,4-triazol-3-yl)propan-2-yl)phenyl)-4-(trifluoromethyl)isoindolin-1-one: (90 mg, 16%, colorless solid, shorter retention time) MS (ESI) calculated for (C₂₁H₁₇F₅N₄O) [M+H]⁺, 436.4; found, 436.8. ¹H NMR (300 MHz, DMSO-d₆) δ 8.44 (s, 1H), 8.11-8.04 (m, 2H), 7.94-7.89 (m, 2H), 7.81 (t, J=7.5 Hz, 1H), 7.44 (t, J=8.1 Hz, 1H), 7.17 (d, J=8.1 Hz, 1H), 6.45-6.23 (m, 1H), 5.19 (s, 2H), 3.54 (s, 3H), 2.00-1.92 (m, 3H), ¹⁹F NMR (300 MHz, DMSO) δ-60.00, −157.46, −157.54, −192.46, −192.54.

Racemic 2-[3-[(1S,2R)-1,2-difluoro-1-(4-methyl-4H-1,2,4-triazol-3-yl)propan-2-yl]phenyl]-4-(trifluoromethyl)-2,3-dihydro-1H-isoindol-1-one: (65 mg, 11%, colorless solid, longer retention time)MS (ESI) calculated for (C₂₁H₁₇F₅N₄O) [M+H]⁺, 436.4; found, 436.8. ¹H NMR (300 MHz, DMSO-d₆) δ 8.45 (s, 1H), 8.12-8.05 (m, 2H), 8.02-7.99 (m, 2H), 7.82 (t, J=7.5 Hz, 1H), 7.50 (t, J=8.1 Hz, 1H), 7.28 (d, J=8.1 Hz, 1H), 6.53-6.31 (m, 1H), 5.25 (s, 2H), 3.51 (s, 3H), 1.87-1.80 (m, 3H), ¹⁹F NMR (300 MHz, DMSO) δ-59.94, −59.98, −73.40, −152.74,-189.49

Step 11: 2-(3-((1R,2R)-1,2-difluoro-1-(4-methyl-4H-1,2,4-triazol-3-yl)propan-2-yl)phenyl)-4-(trifluoromethyl)isoindolin-1-one (478a) and 2-(3-((1S,2S)-1,2-difluoro-1-(4-methyl-4H-1,2,4-triazol-3-yl)propan-2-yl)phenyl)-4-(trifluoromethyl)isoindolin-1-one (478b). The racemic mixture of 2-(3-((1R,2R)-1,2-difluoro-1-(4-methyl-4H-1,2,4-triazol-3-yl)propan-2-yl)phenyl)-4-(trifluoromethyl)isoindolin-1-one (90 mg) was separated by Prep-Chiral-HPLC with the following conditions: [Column: CHIRALPAK IG, 20*250 mm,5 um; Mobile Phase A: MTBE (10 mM NH₃-MeOH)-HPLC, Mobile Phase B: methanol-HPLC; Flow rate: 20 mL/min; Gradient: 50 B to 50 B in 12 min; 220/254 nm] to afford:

2-(3-((1R,2R)-1,2-difluoro-1-(4-methyl-4H-1,2,4-triazol-3-yl)propan-2-yl)phenyl)-4-(trifluoromethyl)isoindolin-1-one (478a): (25.2 mg, colorless solid, longer retention time) MS (ESI) calculated for (C₂₁H₁₇F₅N₄O) [M+H]⁺, 437.1; found, 436.7. ¹H NMR (400 MHz, DMSO-d₆) δ 8.43 (s, 1H), 8.10-8.04 (m, 2H), 7.92-7.89 (m, 2H), 7.80 (t, J=8.0 Hz, 1H), 7.43 (t, J=8.0 Hz, 1H), 7.17 (d, J=7.6 Hz, 1H), 6.42-6.25 (m, 1H), 5.19 (s, 2H), 3.54 (s, 3H), 1.99-1.93 (m, 3H), ¹⁹F NMR (282 MHz, DMSO) δ-59.98, −157.38, −157.41, −192.45, −192.48.

2-(3-((1S,2S)-1,2-difluoro-1-(4-methyl-4H-1,2,4-triazol-3-yl)propan-2-yl)phenyl)-4-(trifluoromethyl)isoindolin-1-one (478b): (19.4 mg, colorless solid, shorter retention time) MS (ESI) calculated for (C₂₁H₁₇F₅N₄O) [M+H]⁺, 437.1; found, 436.7. ¹H NMR (400 MHz, DMSO-d₆) δ 8.43 (s, 1H), 8.10-8.04 (m, 2H), 7.92-7.88 (m, 2H), 7.80 (t, J=8.0 Hz, 1H), 7.443 (t, J=7.6 Hz, 1H), 7.17 (d, J=8.0 Hz, 1H), 6.42-6.25 (m, 1H), 5.19 (s, 2H), 3.53 (s, 3H), 1.99-1.93 (m, 3H), ¹⁹F NMR (282 MHz, DMSO) δ-60.00, −157.37, −157.41, −192.44, −192.48.

Step 12: 2-(3-((1S,2R)-1,2-difluoro-1-(4-methyl-4H-1,2,4-triazol-3-yl)propan-2-yl)phenyl)-4-(trifluoromethyl)isoindolin-1-one (479) and 2-(3-((1R,2S)-1,2-difluoro-1-(4-methyl-4H-1,2,4-triazol-3-yl)propan-2-yl)phenyl)-4-(trifluoromethyl)isoindolin-1-one (480). The racemic mixture of 2-[3-[(1S,2R)-1,2-difluoro-1-(4-methyl-4H-1,2,4-triazol-3-yl)propan-2-yl]phenyl]-4-(trifluoromethyl)-2,3-dihydro-1H-isoindol-1-one (75 mg) was purified by Prep-Chiral-HPLC with following conditions: [Column: CHIRALPAK IG, 20*250 mm,5 um; Mobile Phase A: MTBE (10 mM NH₃-methanol)HPLC, Mobile Phase B: methanol-HPLC; Flow rate: 20 mL/min; Gradient: 50 B to 50 B in 12 min; 220/254 nm] to afford:

2-(3-((1S,2R)-1,2-difluoro-1-(4-methyl-4H-1,2,4-triazol-3-yl)propan-2-yl)phenyl)-4-(trifluoromethyl)isoindolin-1-one (479): (10 mg, colorless solid, shorter retention time)MS (ESI) calculated for (C₂₁H₁₇F₅N₄O) [M+H]⁺, 437.1; found, 437.3. ¹H NMR (400 MHz, DMSO-d₆) δ 8.51 (s, 1H), 8.11-8.05 (m, 2H), 8.00-7.98 (m, 2H), 7.81 (t, J=8.0 Hz, 1H), 7.49 (t, J=8.0 Hz, 1H), 7.28 (d, J=8.0 Hz, 1H), 6.49-6.33 (m, 1H), 5.29-5.19 (m, 2H), 3.51 (s, 3H), 1.86-1.80 (m, 3H), ¹⁹F NMR (376 MHz, DMSO) δ-59.99, −152.65, −189.40, −189.44

2-(3-((1R,2S)-1,2-difluoro-1-(4-methyl-4H-1,2,4-triazol-3-yl)propan-2-yl)phenyl)-4-(trifluoromethyl)isoindolin-1-one (480): MS (11.8 mg, colorless solid, longer retention time) (ESI) calculated for (C₂₁H₁₇F₅N₄O) [M+H]⁺, 437.1; found, 437.2. ¹H NMR (400 MHz, DMSO-d₆) δ 8.51 (s, 1H), 8.11-8.05 (m, 2H), 8.01-7.98 (m, 2H), 7.81 (t, J=8.0 Hz, 1H), 7.49 (t, J=8.0 Hz, 1H), 7.28 (d, J=8.0 Hz, 1H), 6.49-6.33 (m, 1H), 5.29-5.19 (m, 2H), 3.51 (s, 3H), 1.86-1.80 (m, 3H), ¹⁹F NMR (376 MHz, DMSO) δ-59.99, −152.65, −189.40, −189.44

### Examples 481a, 481b, 482, 483: 6-cyclopropyl-2-[3-[(1S,2R)-1,2-difluoro-1-(4-methyl-4H-1,2,4-triazol-3-yl) propan-2-yl]phenyl]-4-(trifluoromethyl)-2,3-dihydro-1H-isoindol-1-one (481a) and 6-cyclopropyl-2-[3-[(1R,2S)-1,2-difluoro-1-(4-methyl-4H-1,2,4-triazol-3-yl) propan-2-yl]phenyl]-4-(trifluoromethyl)-2,3-dihydro-1H-isoindol-1-one (481b)

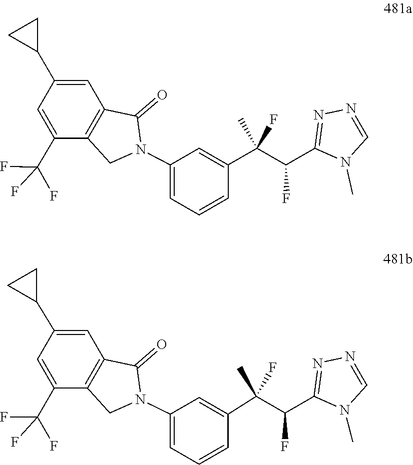 [SMILES: Cn1cnnc1[C@H](F)[C@](C)(F)c1cccc(N2Cc3c(cc(C4CC4)cc3C(F)(F)F)C2=O)c1 ; Cn1cnnc1[C@@H](F)[C@@](C)(F)c1cccc(N2Cc3c(cc(C4CC4)cc3C(F)(F)F)C2=O)c1]

### 6-cyclopropyl-2-[3-[(1R,2R)-1,2-difluoro-1-(4-methyl-4H-1,2,4-triazol-3-yl) propan-2-yl]phenyl]-4-(trifluoromethyl)-2,3-dihydro-1H-isoindol-1-one (482) and 6-cyclopropyl-2-[3-[(1S,2S)-1,2-difluoro-1-(4-methyl-4H-1,2,4-triazol-3-yl) propan-2-yl]phenyl]-4-(trifluoromethyl)-2,3-dihydro-1H-isoindol-1-one (483)

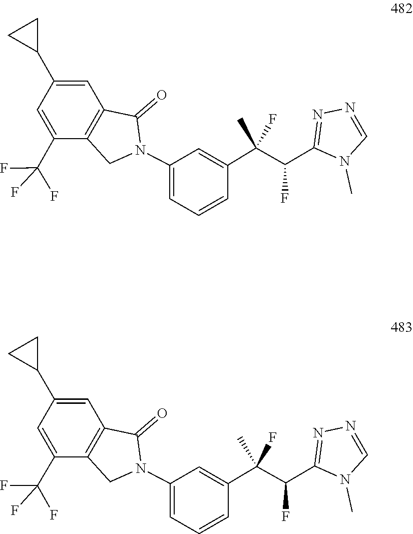 [SMILES: Cn1cnnc1[C@H](F)[C@@](C)(F)c1cccc(N2Cc3c(cc(C4CC4)cc3C(F)(F)F)C2=O)c1 ; Cn1cnnc1[C@@H](F)[C@](C)(F)c1cccc(N2Cc3c(cc(C4CC4)cc3C(F)(F)F)C2=O)c1]

Step 1: Synthesis of 6-bromo-2-(3-(1,2-difluoro-1-(4-methyl-4H-1,2,4-triazol-3-yl)propan-2-yl)phenyl)-4-(trifluoromethyl)isoindolin-1-one. A mixture of methyl 5-bromo-2-(bromomethyl)-3-(trifluoromethyl)benzoate (750 mg, 1.99 mmol), 3-[1,2-difluoro-1-(4-methyl-4H-1,2,4-triazol-3-yl)propan-2-yl]aniline (503 mg, 1.99 mmol) and triethylamine (605.3 mg, 5.98 mmol) in methanol (50 mL) was stirred at 80° C. for 16 h. The solvent was removed under vacuum to give residue, which was purified by reverse phase flash column chromatography with 10-64% acetonitrile in water to afford the title compound (300 mg, 29%) as a yellow solid. MS (ESI) calculated for (C₂₁H₁₆BrF₅N₄O) [M+H]⁺, 515.0; found, 515.0.

Step 2: Synthesis of trans-6-cyclopropyl-2-(3-((1S,2R)-1,2-difluoro-1-(4-methyl-4H-1,2,4-triazol-3-yl)propan-2-yl)phenyl)-4-(trifluoromethyl)isoindolin-1-one and cis-6-cyclopropyl-2-(3-((1R,2R)-1,2-difluoro-1-(4-methyl-4H-1,2,4-triazol-3-yl)propan-2-yl)phenyl)-4-(trifluoromethyl)isoindolin-1-one. To a degassed solution of 6-bromo-2-[3-[1,2-difluoro-1-(4-methyl-4H-1,2,4-triazol-3-yl)propan-2-yl]phenyl]-4-(trifluoromethyl)-2,3-dihydro-1H-isoindol-1-one (340 mg, 0.66 mmol) in toluene (10 mL) and water (1 mL) were added cyclopropylboronic acid (90.7 mg, 1.06 mmol), tricyclohexylphosphane (20.4 mg, 0.07 mmol), tripotassium phosphate (700.3 mg, 3.30 mmol) and palladium acetate (7.4 mg, 0.03 mmol). The solution was stirred at 100° C. for 16 h then concentrated. The residue was diluted with water (30 mL) and the aqueous phase was extracted with EtOAc (30 mL×3). The combined organic layers were washed with brine, dried, filtered and concentrated. The residue was purified by reverse phase flash column chromatography with 5-55% acetonitrile in water to afford a mixture of containing four isomers of 6-cyclopropyl-2-[3-[1,2-difluoro-1-(4-methyl-4H-1,2,4-triazol-3-yl)propan-2-yl]phenyl]-4-(trifluoromethyl)-2,3-dihydro-1H-isoindol-1-one (180 mg, 57%) as a colorless solid. The mixture was then separated by prep-HPLC to afford trans-6-cyclopropyl-2-[3-[(1 S,2R)-1,2-difluoro-1-(4-methyl-4H-1,2,4-triazol-3-yl) propan-2-yl]phenyl]-4-(trifluoromethyl)-2,3-dihydro-1H-isoindol-1-one (70 mg) as a colorless solid with shorter retention time on HPLC and cis-6-cyclopropyl-2-[3-[(1R,2R)-1,2-difluoro-1-(4-methyl-4H-1,2,4-triazol-3-yl) propan-2-yl]phenyl]-4-(trifluoromethyl)-2,3-dihydro-1H-isoindol-1-one (70 mg) as a colorless solid with longer retention time on HPLC. MS (ESI) calculated for (C₂₄H₂₁F₅N40) [M+H]⁺, 477.2; found, 476.8.

Step 3: 6-cyclopropyl-2-[3-[(1S,2R)-1,2-difluoro-1-(4-methyl-4H-1,2,4-triazol-3-yl) propan-2-yl]phenyl]-4-(trifluoromethyl)-2,3-dihydro-1H-isoindol-1-one (481a) and 6-cyclopropyl-2-[3-[(1R,2S)-1,2-difluoro-1-(4-methyl-4H-1,2,4-triazol-3-yl) propan-2-yl]phenyl]-4-(trifluoromethyl)-2,3-dihydro-1H-isoindol-1-one (481b). The trans-isomer of 6-cyclopropyl-2-[3-[(1 S,2R)-1,2-difluoro-1-(4-methyl-4H-1,2,4-triazol-3-yl)propan-2-yl]phenyl]-4-(trifluoromethyl)-2,3-dihydro-1H-isoindol-1-one (70 mg) was separated by Chiral-Prep-HPLC with the following conditions: [Column: CHIRALPAK IC, 2*25 cm,5 um; Mobile Phase A: MTBE (10 mM NH₃-MeOH)-HPLC, Mobile Phase B: EtOH-HPLC; Flow rate: 20 mL/min; Gradient: 30% B to 30% B in 14 min; 220/254 nm] to afford:

6-cyclopropyl-2-[3-[(1S,2R)-1,2-difluoro-1-(4-methyl-4H-1,2,4-triazol-3-yl)propan-2-yl]phenyl]-4-(trifluoromethyl)-2,3-dihydro-1H-isoindol-1-one (481a): (11.2 mg, colorless solid, shorter retention time) MS (ESI) calculated for (C₂₄H₂₁F₅N₄O) [M+H]⁺, 477.2; found, 476.8. ¹H NMR (300 MHz, DMSO-d6) δ 8.43 (s, 1H), 7.93-7.90 (m, 1H), 7.85 (s, 1H), 7.77 (s, 1H), 7.71 (s, 1H), 7.43 (t, J=8.1 Hz, 1H), 7.16 (d, J=8.1 Hz, 1H), 6.53-6.23 (m, 1H), 5.11 (s, 2H), 3.54 (s, 3H), 2.28-2.22 (m, 1H), 1.99-1.91 (m, 3H), 1.13-1.06 (m, 2H), 0.91-0.84 (m, 2H). ¹⁹F NMR (282 MHz, DMSO-d6) 5-59.91, −157.49, −157.54, −192.47, −192.52.

6-cyclopropyl-2-[3-[(1R,2S)-1,2-difluoro-1-(4-methyl-4H-1,2,4-triazol-3-yl)propan-2-yl]phenyl]-4-(trifluoromethyl)-2,3-dihydro-1H-isoindol-1-one (481b): (7.4 mg, colorless solid, longer retention time) MS (ESI) calculated for (C₂₄H₂₁F₅N₄O) [M+H]⁺, 477.2; found, 476.8. ¹H NMR (300 MHz, DMSO-d6) δ 8.43 (s, 1H), 7.93-7.90 (m, 1H), 7.85 (s, 1H), 7.77 (s, 1H), 7.71 (s, 1H), 7.43 (t, J=8.1 Hz, 1H), 7.16 (d, J=7.8 Hz, 1H), 6.53-6.23 (m, 1H), 5.11 (s, 2H), 3.54 (s, 3H), 2.28-2.20 (m, 1H), 2.00-1.91 (m, 3H), 1.12-1.07 (m, 2H), 0.92-0.85 (m, 2H). ¹⁹F NMR (282 MHz, DMSO) δ-59.91, −157.49, −157.54, −192.48, −192.53.

Step 4 6-cyclopropyl-2-[3-[(1R,2R)-1,2-difluoro-1-(4-methyl-4H-1,2,4-triazol-3-yl) propan-2-yl]phenyl]-4-(trifluoromethyl)-2,3-dihydro-1H-isoindol-1-one (483) and 6-cyclopropyl-2-[3-[(1S,2S)-1,2-difluoro-1-(4-methyl-4H-1,2,4-triazol-3-yl) propan-2-yl]phenyl]-4-(trifluoromethyl)-2,3-dihydro-1H-isoindol-1-one (482). The cis-isomer of 6-cyclopropyl-2-[3-[(1R,2R)-1,2-difluoro-1-(4-methyl-4H-1,2,4-triazol-3-yl)propan-2-yl]phenyl]-4-(trifluoromethyl)-2,3-dihydro-1H-isoindol-1-one (70 mg) was separated by chiral-Prep-HPLC with the following conditions: [Column: Chiralpak IC, 2*25 cm, 5 um; Mobile Phase A: MTBE (10 mM NH₃-methanol)-HPLC, Mobile Phase B: EtOH-HPLC; Flow rate: 20 mL/min; Gradient: 30 B to 30 B in 15 min; 220/254 nm] to afford:

6-cyclopropyl-2-[3-[(1R,2R)-1,2-difluoro-1-(4-methyl-4H-1,2,4-triazol-3-yl)propan-2-yl]phenyl]-4-(trifluoromethyl)-2,3-dihydro-1H-isoindol-1-one (483): (21.6 mg, colorless solid, shorter retention time) MS (ESI) calculated for (C₂₄H₂₁F₅N₄O) [M+H]⁺, 477.2; found, 476.8. ¹H NMR (300 MHz, DMSO-d₆) δ 8.52 (s, 1H), 8.02-7.99 (m, 2H), 7.77-7.73 (m, 2H), 7.48 (t, J=7.8 Hz, 1H), 7.27 (d, J=7.8 Hz, 1H), 6.52-6.31 (m, 1H), 5.17 (s, 2H), 3.51 (s, 3H), 2.29-2.22 (m, 1H), 1.89-1.79 (m, 3H), 1.13-1.09 (m, 2H), 0.89-0.82 (m, 2H). ¹⁹F NMR (300 MHz, DMSO) δ-59.89, −152.70, −189.41, −189.46.

6-cyclopropyl-2-[3-[(1S,2S)-1,2-difluoro-1-(4-methyl-4H-1,2,4-triazol-3-yl)propan-2-yl]phenyl]-4-(trifluoromethyl)-2,3-dihydro-1H-isoindol-1-one (482): (16 mg, colorless solid, longer retention time) MS (ESI) calculated for (C₂₄H₂₁F₅N₄O) [M+H]⁺, 477.2; found, 476.8. ¹H NMR (300 MHz, DMSO-d₆) δ 8.52 (s, 1H), 8.02-7.99 (m, 2H), 7.77 (s, 1H), 7.73 (s, 1H), 7.48 (t, J=7.8 Hz, 1H), 7.27 (d, J=7.8 Hz, 1H), 6.52-6.31 (m, 1H), 5.17 (s, 2H), 3.37 (s, 3H), 2.29-2.22 (m, 1H), 1.87-1.79 (m, 3H), 1.13-1.08 (m, 2H), 0.91-0.86 (m, 2H). ¹⁹F NMR (300 MHz, DMSO) δ-59.89, −152.66, −189.42, −189.48.

### Example 484: 6-[(3S)-3-hydroxypyrrolidine-1-carbonyl]-4-(trifluoromethyl)-2-[3-[(2R)-1-[4-(trifluoromethyl)-1,2-oxazol-3-yl]propan-2-yl]phenyl]-2,3-dihydro-1H-isoindol-1-one

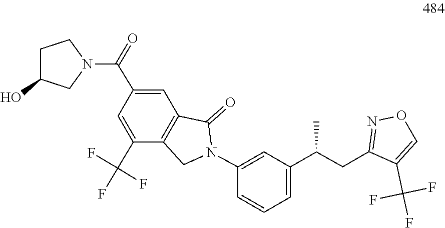 [SMILES: C[C@H](Cc1nocc1C(F)(F)F)c1cccc(N2Cc3c(cc(C(=O)N4CC[C@H](O)C4)cc3C(F)(F)F)C2=O)c1]

(484)

Step 1: Synthesis of tert-butyl N-[3-[(2R)-4-hydroxybutan-2-yl]phenyl]carbamate. To a solution of LiAlH₄ (39 mL, 2.5 M in THF) in THF (100 mL) was added a solution of ethyl (3R)-3-(3-[[(tert-butoxy)carbonyl]amino]phenyl)butanoate (20.0 g, 65.1 mmol) in THF (150 mL) slowly at −30° C. under nitrogen. The mixture was stirred at −30° C. for 1 h. The reaction was quenched by the addition of water carefully and then diluted by EtOAc. The solids were filtered off. The filtrate was washed with water, dried, filtered and concentrated to afford the title compound (18.0 g, crude) as a yellow oil, which was used without purification. MS (ESI) calculated for (C₁₅H₂₃NO₃) [M+Na]⁺, 288.3; found, 288.2.

Synthesis of tert-butyl N-[3-[(2R)-4-oxobutan-2-yl]phenyl]carbamate. To a solution of tert-butyl N-[3-[(2R)-4-hydroxybutan-2-yl]phenyl]carbamate (10.0 g, 37.7 mmol) in EtOAc (500 mL) was added IBX (21.0 g, 75.0 mmol) at rt. The mixture was heated at reflux for 16 h. The solids were filtered off. The filtrate was concentrated to afford the title compound (12.0 g, crude) as brown oil, which was used without purification. MS (ESI) calculated for (C₄₅H₂₁NO₃) [M+Na]⁺, 286.3; found, 286.2.

Step 3: Synthesis of tert-butyl N-[3-[(2R)-4-(hydroxyimino)butan-2-yl]phenyl]carbamate. To a solution of tert-butyl N-[3-[(2R)-4-oxobutan-2-yl]phenyl]carbamate (12.0 g, 45.6 mmol) in methanol (120 mL) were added water (50 mL), NH₂OH.HCl (4.7 g, 68.3 mmol) and Na₂CO₃ (7.2 g, 68.3 mmol) at rt. The mixture was stirred at rt for 3 h. The mixture was diluted with water and extracted with EtOAc. The organic layers were dried, and concentrated to afford the title compound (12.6 g, crude) as brown oil, which was used without purification. MS (ESI) calculated for (C₁₅H₂₂N₂O₃) [M+H]⁺, 279.2; found, 279.0.

Step 4: Synthesis of tert-butyl N-[3-[(2R)-4-chloro-4-(hydroxyimino)butan-2-yl]phenyl]carbamate. To a solution of tert-butyl N-[3-[(2R)-4-(hydroxyimino)butan-2-yl]phenyl]carbamate (1.0 g, 3.6 mmol) in DMF (10 mL) was added NCS (481 mg, 3.6 mmol) at 0° C. The mixture was stirred at rt for 2 h. The mixture was diluted with water and extracted with EtOAc. The organic layers were dried, filtered and concentrated to afford the title compound (1.2 g, crude) as a yellow oil, which was used without purification. MS (ESI) calculated for (C₁₅H₂₁ClN₂O₃) [M+H]⁺, 313.1; found, 313.1.

Step 5: Synthesis of tert-butyl N-[3-[(2R)-1-[5-methoxy-4-(trifluoromethyl)-4,5-dihydro-1,2-oxazol-3-yl]propan-2-yl]phenyl]carbamate. To a solution of tert-butyl N-[3-[(2R)-4-chloro-4-(hydroxyimino)butan-2-yl]phenyl]carbamate (1.6 g, crude) and (1E)-3,3,3-trifluoro-1-methoxyprop-1-ene (0.6 g, 5.1 mmol) in i-PrOH (20 mL) was added NaHCO₃ (0.9 g, 10.2 mmol) at rt. The mixture was stirred at rt for 16 h. The mixture was diluted with water and extracted with EtOAc. The organic layers were dried, filtered and concentrated. The residue was purified by flash column chromatography with 0-30% EtOAc in petroleum ether to afford the title compound (700 mg, 34%) as a light yellow syrup. MS (ESI) calculated for (C₁₉H₂₅F₃N₂O₄) [M+H]⁺, 403.2; found, 403.3.

Step 6: Synthesis of 3-[(2R)-1-[4-(trifluoromethyl)-1,2-oxazol-3-yl]propan-2-yl]aniline. To a solution of tert-butyl N-[3-[(2R)-1-[5-methoxy-4-(trifluoromethyl)-4,5-dihydro-1,2-oxazol-3-yl]propan-2-yl]phenyl]carbamate (650 mg, 1.6 mmol) in methanol (10 mL) was added HCl (conc., 2 mL). Then the mixture was heated at 70° C. for 4 h. The mixture was basified by NaOH (aq.) to pH 8, and then extracted with EtOAc. The combined organic layers were dried, filtered and concentrated. The residue was purified by reverse phase flash column chromatography with 10-70% MeCN in water (0.5% formic acid) to afford the title compound (180 mg, 41%) as a yellow oil. MS (ESI) calculated for (C₁₃H₁₃F₃N₂O) [M+H]⁺, 271.1; found, 271.1.

Step 7: Synthesis of 3-oxo-7-(trifluoromethyl)-2-[3-[(2R)-1-[4-(trifluoromethyl)-1,2-oxazol-3-yl]propan-2-yl]phenyl]-2,3-dihydro-1H-isoindole-5-carboxylic acid. The indolone formation reaction was carried out in a manner similar to 260, step 2 using 3-[(2R)-1-[4-(trifluoromethyl)-1,2-oxazol-3-yl]propan-2-yl]aniline (90 mg, 0.3 mmol) and 4-(bromomethyl)-3-(methoxycarbonyl)-5-(trifluoromethyl)benzoic acid (120 mg, 0.4 mmol) to afford the title compound (70 mg, 42%) as a yellow syrup. MS (ESI) calculated for (C₂₃H₁₆F₆N₂O₄) [M+H]⁺, 499.1; found, 499.3.

Step 8: Synthesis of 6-[(3S)-3-hydroxypyrrolidine-1-carbonyl]-4-(trifluoromethyl)-2-[3-[(2R)-1-[4-(trifluoromethyl)-1,2-oxazol-3-yl]propan-2-yl]phenyl]-2,3-dihydro-1H-isoindol-1-one (484). 3-oxo-7-(trifluoromethyl)-2-[3-[(2R)-1-[4-(trifluoromethyl)-1,2-oxazol-3-yl]propan-2-yl]phenyl]-2,3-dihydro-1H-isoindole-5-carboxylic acid (70 mg, 0.2 mmol) and (3S)-pyrrolidin-3-ol hydrochloride (21 mg, 0.2 mmol) were coupled in a manner similar to 184 to the title compound (20 mg, 25%) as a colorless solid. MS (ESI) calculated for (C₂₇H₂₃F₆N₃O₄) [M+H]⁺, 568.2; found, 568.2. ¹H NMR (400 MHz, DMSO-d₆) δ 9.68 (s, 1H), 8.13-8.11 (m, 2H), 7.89 (s, 1H), 7.82-7.74 (m, 1H), 7.39 (t, J=7.8 Hz, 1H), 7.14 (d, J=7.8 Hz, 1H), 5.27 (s, 2H), 5.08-5.01 (m, 1H), 4.36-4.27 (m, 1H), 3.69-3.54 (m, 2H), 3.47-3.42 (m, 1H), 3.29-3.22 (m, 2H), 3.10-3.04 (m, 2H), 1.99-1.82 (m, 2H), 1.33 (d, J=6.9 Hz, 3H). ¹⁹F NMR (400 MHz, DMSO-d₆) δ-56.118, 60.093.

### Example 485: 6-[[(3S)-3-fluoropyrrolidin-1-yl]methyl]-4-(trifluoromethyl)-2-[3-[(2R)-1-[4-(trifluoromethyl)-1,2-oxazol-3-yl]propan-2-yl]phenyl]-2,3-dihydro-1H-isoindol-1-one (485)

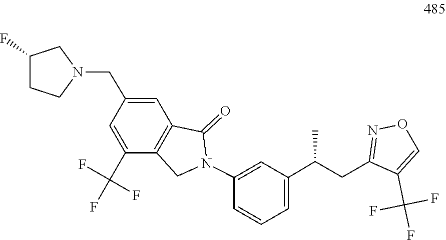 [SMILES: C[C@H](Cc1nocc1C(F)(F)F)c1cccc(N2Cc3c(cc(CN4CC[C@H](F)C4)cc3C(F)(F)F)C2=O)c1]

Step 1: Synthesis of methyl 2-(bromomethyl)-5-[[(3S)-3-fluoropyrrolidin-1-yl]methyl]-3-(trifluoromethyl)benzoate. To a solution of methyl 2-(bromomethyl)-5-formyl-3-(trifluoromethyl)benzoate (150 mg, 0.5 mmol) and (3S)-3-fluoropyrrolidine (45 mg, 0.5 mmol) in methylene chloride (5 mL) were added NaBH(OAc)₃ (195 mg, 0.9 mmol) and AcOH (0.5 mL). The mixture was stirred at rt for 16 h. The mixture was diluted with water and extracted with methylene chloride (20 mL×3). The combined organic layers were washed with water, dried, filtered and concentrated to afford the title compound (105 mg, crude) as a yellow oil, which was used without purification. MS (ESI) calculated for (C₁₅H₁₆BrF₄NO₂) [M+H]⁺, 398.0, 400.0; found, 398.1, 400.1.

Step 2: Synthesis of 6-[[(3S)-3-fluoropyrrolidin-1-yl]methyl]-4-(trifluoromethyl)-2-[3-[(2R)-1-[4-(trifluoromethyl)-1,2-oxazol-3-yl]propan-2-yl]phenyl]-2,3-dihydro-1H-isoindol-1-one (485): A mixture of methyl 2-(bromomethyl)-5-[[(3S)-3-fluoropyrrolidin-1-yl]methyl]-3-(trifluoromethyl)benzoate (105 mg, 0.3 mmol), 3-[(2R)-1-[4-(trifluoromethyl)-1,2-oxazol-3-yl]propan-2-yl]aniline (90 mg, 0.3 mmol) and AgNO₃ (134 mg, 0.8 mmol) in methanol (5 mL) was heated at 60° C. for 2 h. The mixture was filtered, and concentrated The residue was purified by Prep-HPLC to afford the title compound (9.5 mg, 6%) as a light yellow semi-solid. MS (ESI) calculated for (C₂₇H₂₄F₇N₃O₂) [M+H]⁺, 556.2; found, 556.2. ¹H NMR (300 MHz, Methanol-d₄) δ 9.17 (s, 1H), 8.08 (s, 1H), 7.98 (s, 1H), 7.83 (s, 1H), 7.67-7.64 (m, 1H), 7.39 (t, J=7.8 Hz, 1H), 7.14 (d, J=7.8 Hz, 1H), 5.31-5.21 (m, 1H), 5.18-5.05 (m, 2H), 3.93-3.89 (m, 2H), 3.41-3.30 (m, 1H), 3.21-3.01 (m, 2H), 3.01-2.66 (m, 3H), 2.56-2.48 (m, 1H), 2.52-2.18 (m, 2H), 1.39 (d, J=6.9 Hz, 3H). ¹⁹F NMR (282 MHz, Methanol-d₄) δ-58.779, −62.936, −169.480.

### Example 486a and 486b: 6-[(1R,5S)-2-azabicyclo[3.1.0]hexan-2-ylmethyl]-2-(3-{3-[(4-methyl-1,2,4-triazol-3-yl)methyl]oxetan-3-yl}phenyl)-4-(trifluoromethyl)-3H-isoindol-1-one and 6-[(1S,5R)-2-azabicyclo[3.1.0]hexan-2-ylmethyl]-2-(3-{3-[(4-methyl-1,2,4-triazol-3-yl)methyl]oxetan-3-yl}phenyl)-4-(trifluoromethyl)-3H-isoindol-1-one

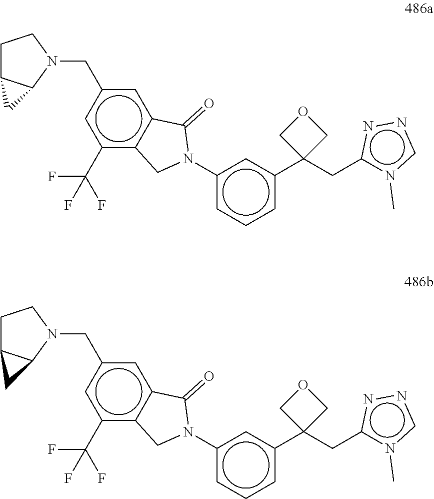

Example Z (80 mg, 0.1 mmol) and 3-azabicyclo[3.1.0]hexane hydrochloride (105 mg, 0.88 mmol, were coupled in a similar fashion as for 447, step 4 to afford racemic title compound which was resolved by SFC: First peak: ¹H NMR (500 MHz, DMSO-d₆) δ 8.18 (s, 1H), 7.99 (d, J=18.2 Hz, 2H), 7.93-7.84 (m, 1H), 7.40 (t, J=2.0 Hz, 1H), 7.35 (t, J=7.9 Hz, 1H), 6.76 (dt, J=7.6, 1.3 Hz, 1H), 5.10 (s, 2H), 4.96 (d, J=6.0 Hz, 2H), 4.89 (d, J=6.0 Hz, 2H), 3.86 (d, J=13.5 Hz, 1H), 3.75 (d, J=13.5 Hz, 1H), 3.51 (s, 2H), 2.90 (s, 3H), 2.76 (t, J=8.4 Hz, 1H), 2.59 (td, J=5.8, 2.6 Hz, 2H), 2.01-1.84 (m, 2H), 1.79 (dd, J=11.6, 6.9 Hz, 1H), 1.43-1.33 (m, 1H), 0.73 (ddd, J=5.5, 4.2, 2.6 Hz, 1H), 0.08 (dt, J=8.1, 5.5 Hz, 1H); LCMS: C₂₈H₂₈F₃N₅O₂ requires: 524, found: m/z=525 [M+H]⁺.

Second Peak ¹H NMR (500 MHz, DMSO-d₆) δ 8.18 (s, 1H), 8.00 (s, 1H), 7.97 (s, 1H), 7.88 (ddd, J=8.2, 2.3, 0.9 Hz, 1H), 7.40 (t, J=2.0 Hz, 1H), 7.35 (t, J=7.9 Hz, 1H), 6.76 (dt, J=7.8, 1.1 Hz, 1H), 5.10 (s, 2H), 4.96 (d, J=6.1 Hz, 2H), 4.89 (d, J=6.0 Hz, 2H), 3.86 (d, J=13.5 Hz, 1H), 3.75 (d, J=13.5 Hz, 1H), 3.51 (s, 2H), 2.90 (s, 3H), 2.76 (t, J=8.8 Hz, 1H), 2.59 (td, J=5.8, 2.6 Hz, 1H), 2.01-1.84 (m, 2H), 1.79 (dd, J=11.6, 6.8 Hz, 1H), 1.39 (ddd, J=10.2, 8.3, 4.3 Hz, 1H), 0.73 (ddd, J=5.6, 4.1, 2.6 Hz, 1H), 0.08 (dt, J=8.2, 5.6 Hz, 1H); LCMS: C₂₈H₂₈F₃N₅O₂ requires: 524, found: m/z=525 [M+H]⁺.

### Example 487: 6-{5-azaspiro[2.3]hexan-5-ylmethyl}-2-(3-{3-[(4-methyl-1,2,4-triazol-3-yl)methyl]oxetan-3-yl}phenyl)-4-(trifluoromethyl)-3H-isoindol-1-one

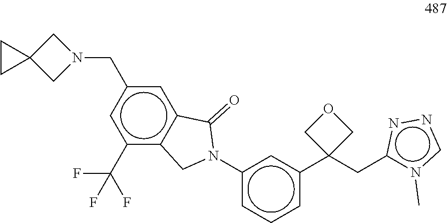

Example Z (115 mg, 0.25 mmol) and 5-azaspiro[2.3]hexane hydrochloride (30 mg, 0.25 mmol) were coupled in a similar fashion as for 447, step 4 to afford the title compound as an off-white solid: ¹H NMR (500 MHz, DMSO-d₆) δ 8.18 (s, 1H), 7.97 (s, 1H), 7.92 (s, 1H), 7.90-7.85 (m, 1H), 7.39 (t, J=1.9 Hz, 1H), 7.35 (t, J=8.0 Hz, 1H), 6.76 (dt, J=7.9, 1.2 Hz, 1H), 5.09 (s, 2H), 4.96 (d, J=6.1 Hz, 2H), 4.88 (d, J=6.1 Hz, 2H), 3.88 (s, 2H), 3.51 (s, 2H), 2.90 (s, 3H), 0.52 (s, 4H); LCMS: C₂₈H₂₈F₃N₅O₂ requires: 524, found: m/z=525 [M+H]⁺.

### Example 488: 6-({6,6-difluoro-3-azabicyclo[3.1.0]hexan-3-yl}methyl)-2-(3-{3-[(4-methyl-1,2,4-triazol-3-yl)methyl]oxetan-3-yl}phenyl)-4-(trifluoromethyl)-3H-isoindol-1-one

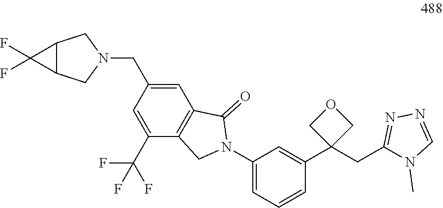 [SMILES: Cn1cnnc1CC1(c2cccc(N3Cc4c(cc(CN5CC6C(C5)C6(F)F)cc4C(F)(F)F)C3=O)c2)COC1]

Example Z (35 mg, 0.08 mmol) and 6,6-difluoro-3-azabicyclo[3.1.0]hexane hydrochloride (47 mg, 0.31 mmol) were coupled in a similar fashion as for 447, step 4 to afford the title compound as an off-white solid: ¹H NMR (500 MHz, DMSO-d₆) δ 8.19 (s, 1H), 8.09-7.90 (m, 2H), 7.88 (dd, J=8.0, 2.2 Hz, 1H), 7.40 (t, J=2.0 Hz, 1H), 7.35 (t, J=7.9 Hz, 1H), 6.77 (d, J=7.6 Hz, 1H), 5.10 (s, 2H), 4.96 (d, J=6.0 Hz, 2H), 4.89 (d, J=6.1 Hz, 2H), 3.87 (br s, 2H), 3.51 (s, 2H), 3.05 (s, 2H), 3.11-2.97 (br s, 2H), 2.91 (s, 3H), 2.42-2.26 (br s, 2H); LCMS: C₂₈H₂₆F₅N₅O₂ requires: 560, found: m/z=561 [M+H]⁺.

### Example 489: 6-[(4-fluoropiperidin-1-yl)methyl]-2-(3-{3-[(4-methyl-1,2,4-triazol-3-yl)methyl]oxetan-3-yl}phenyl)-4-(trifluoromethyl)-3H-isoindol-1-one

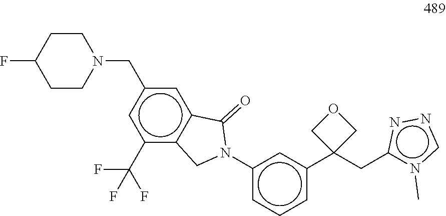

Example Z (100 mg, 0.25 mmol) and 4-fluoropiperidine hydrochloride (47 mg, 0.31 mmol) were coupled in a similar fashion as for 447, step 4 to afford the title compound as an off-white solid: ¹H NMR (500 MHz, DMSO-d₆) δ 8.18 (s, 1H), 7.99 (s, 1H), 7.93 (s, 1H), 7.88 (ddd, J=8.3, 2.2, 1.0 Hz, 1H), 7.39 (t, J=2.0 Hz, 1H), 7.35 (t, J=8.0 Hz, 1H), 6.76 (dt, J=7.8, 1.2 Hz, 1H), 5.10 (s, 2H), 4.96 (d, J=6.0 Hz, 2H), 4.89 (d, J=6.1 Hz, 2H), 4.79-4.60 (m, 1H), 3.70 (s, 2H), 3.51 (s, 2H), 2.90 (s, 3H), 2.35 (s, 3H), 1.93-1.79 (m, 2H), 1.79-1.66 (m, 2H); LCMS: C₂₈H₂₉F₄N₅O₂ requires: 543, found: m/z=544 [M+H]⁺.

### Example 490: 6-((2-azabicyclo[4.1.0]heptan-2-yl)methyl)-2-(3-(3-((4-methyl-4H-1,2,4-triazol-3-yl)methyl)oxetan-3-yl)phenyl)-4-(trifluoromethyl)isoindolin-1-one

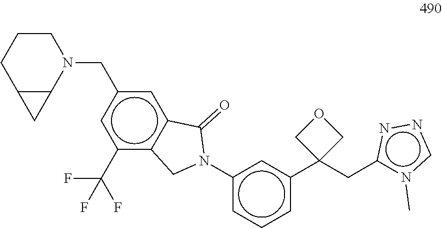

Example Z (50 mg, 0.11 mmol) and 2-azabicyclo[4.1.0]heptane hydrochloride (30 mg, 0.22 mmol) were coupled in a similar fashion as for 447, step 4 to afford the title compound as an off-white solid: ¹H NMR (500 MHz, DMSO-d₆) δ 8.18 (s, 1H), 8.01 (s, 1H), 7.97 (s, 1H), 7.88 (ddd, J=8.2, 2.3, 0.9 Hz, 1H), 7.40 (t, J=2.0 Hz, 1H), 7.35 (t, J=7.9 Hz, 1H), 6.76 (dt, J=7.9, 1.2 Hz, 1H), 5.10 (s, 2H), 4.96 (d, J=6.0 Hz, 2H), 4.89 (d, J=6.0 Hz, 2H), 3.88 (d, J=13.9 Hz, 1H), 3.81 (d, J=13.8 Hz, 1H), 3.51 (s, 2H), 2.90 (s, 3H), 2.47-2.43 (m, 1H), 2.22-2.10 (m, 2H), 1.96-1.86 (m, 1H), 1.56-1.44 (m, 1H), 1.44-1.32 (m, 2H), 1.00 (tt, J=8.3, 6.2 Hz, 1H), 0.35-0.23 (m, 2H); LCMS: C₂₉H₃₀F₃N₅O₂ requires: 538, found: m/z=539 [M+H]⁺.

### Example 491: 6-((1-methyl-3-azabicyclo[3.1.0]hexan-3-yl)methyl)-2-(3-(3-((4-methyl-4H-1,2,4-triazol-3-yl)methyl)oxetan-3-yl)phenyl)-4-(trifluoromethyl)isoindolin-1-one

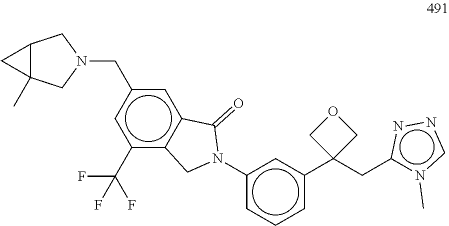

Example Z (50 mg, 0.11 mmol) and 1-methyl-3-azabicyclo[3.1.0]hexane hydrochloride (37 mg, 0.27 mmol) were coupled in a similar fashion as for 447, step 4 to afford the title compound as an off-white solid: ¹H NMR (500 MHz, DMSO-d₆) δ 8.18 (s, 1H), 7.92 (s, 1H), 7.90-7.84 (m, 2H), 7.40 (t, J=1.9 Hz, 1H), 7.35 (t, J=8.0 Hz, 1H), 6.76 (d, J=7.7 Hz, 1H), 5.09 (s, 2H), 4.96 (d, J=6.0 Hz, 2H), 4.89 (d, J=6.1 Hz, 2H), 3.84-3.75.74172 (m, 2H), 3.51 (s, 2H), 2.90 (s, 3H), 2.84 (t, J=8.8 Hz, 2H), 2.42 (dd, J=8.6, 3.5 Hz, 1H), 2.21 (d, J=8.4 Hz, 1H), 1.18 (s, 3H), 1.09 (dt, J=7.3, 3.5 Hz, 1H), 0.86 (t, J=3.7 Hz, 1H), 0.28 (dd, J=7.7, 3.7 Hz, 1H); LCMS: C₂₉H₃₀F₃N₅O₂ requires: 538, found: m/z=539 [M+H]⁺.

### Example 492: N-(3-{3-[(4-methyl-1,2,4-triazol-3-yl)methyl]oxetan-3-yl}phenyl)-2-(trifluoromethyl)-1,3-thiazole-4-carboxamide

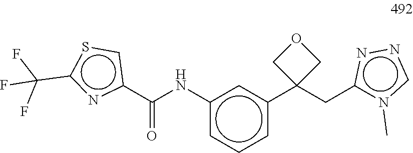

3-{3-[(4-methyl-1,2,4-triazol-3-yl)methyl]oxetan-3-yl}aniline (30 mg, 0.12 mmol) and 2-(trifluoromethyl)-1,3-thiazole-4-carboxylic acid (24 mg, 0.12 mmol) were coupled in a similar fashion as for 74, to afford 24 mg (48%) of the title compound as an off-white solid: ¹H NMR (500 MHz, DMSO-d₆) δ 10.36 (s, 1H), 8.83 (s, 1H), 8.20 (s, 1H), 7.83-7.73 (m, 1H), 7.42 (t, J=1.9 Hz, 1H), 7.26 (t, J=8.0 Hz, 1H), 6.67 (dt, J=7.8, 1.3 Hz, 1H), 4.93 (d, J=5.9 Hz, 2H), 4.84 (d, J=5.9 Hz, 2H), 3.48 (s, 2H), 2.90 (s, 3H); LCMS: C₁₈H₁₆F₃N₅O₂S requires: 423, found: m/z=424 [M+H]⁺.

### Example 493: 4-cyclopropyl-N-(3-{3-[(4-methyl-1,2,4-triazol-3-yl)methyl]oxetan-3-yl}phenyl)-1,3-thiazole-2-carboxamide

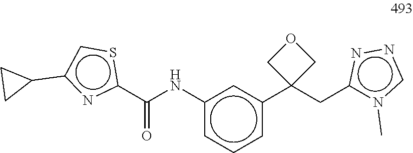

3-{3-[(4-methyl-1,2,4-triazol-3-yl)methyl]oxetan-3-yl}aniline (30 mg, 0.12 mmol) and sodium 4-cyclopropyl-1,3-thiazole-2-carboxylate (24 mg, 0.12 mmol) were coupled in a similar fashion as for 74, to afford 21 mg (43%) of the title compound as an off-white solid: ¹H NMR (500 MHz, DMSO-d₆) δ 10.35 (s, 1H), 8.20 (s, 1H), 7.74 (dd, J=8.1, 2.0 Hz, 1H), 7.65 (s, 1H), 7.45 (t, J=1.9 Hz, 1H), 7.26 (t, J=7.9 Hz, 1H), 6.67 (dd, J=7.8, 1.3 Hz, 1H), 4.93 (d, J=5.9 Hz, 2H), 4.84 (d, J=6.0 Hz, 2H), 3.47 (s, 2H), 2.91 (s, 3H), 2.19 (tt, J=8.1, 5.1 Hz, 1H), 1.04-0.87 (m, 4H); LCMS: C₂₀H₂₁N₅O₂S requires: 395, found: m/z=396 [M+H]⁺.

### Example 494: 2-cyclopropyl-N-(3-{3-[(4-methyl-1,2,4-triazol-3-yl)methyl]oxetan-3-yl}phenyl)-1,3-thiazole-4-carboxamide

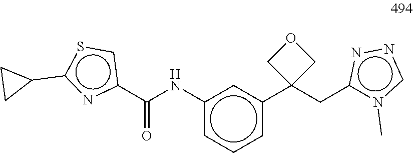

3-{3-[(4-methyl-1,2,4-triazol-3-yl)methyl]oxetan-3-yl}aniline (30 mg, 0.12 mmol) and 2-cyclopropyl-1,3-thiazole-4-carboxylic acid (21 mg, 0.12 mmol) were coupled in a similar fashion as for 74, to afford 27 mg (56%) of the title compound as an off-white solid: ¹H NMR (500 MHz, DMSO-d₆) δ 9.93 (s, 1H), 8.19 (s, 1H), 8.16 (s, 1H), 7.75 (ddd, J=8.1, 2.1, 0.9 Hz, 1H), 7.41 (t, J=1.9 Hz, 1H), 7.24 (t, J=7.9 Hz, 1H), 6.62 (ddd, J=7.8, 1.8, 1.0 Hz, 1H), 4.92 (d, J=5.9 Hz, 2H), 4.84 (d, J=5.9 Hz, 2H), 3.47 (s, 2H), 2.90 (s, 3H), 1.25-1.14 (m, 2H), 1.14-1.03 (m, 2H); LCMS: C₂₀H₂₁N₅O₂S requires: 395, found: m/z=396 [M+H]⁺.

### Example 495: N-(3-{3-[(4-methyl-1,2,4-triazol-3-yl)methyl]oxetan-3-yl}phenyl)-4-(trifluoromethyl)-1,3-thiazole-2-carboxamide

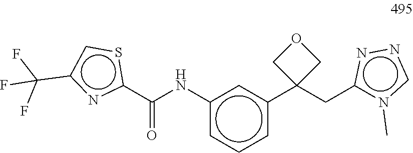

3-{3-[(4-methyl-1,2,4-triazol-3-yl)methyl]oxetan-3-yl}aniline (30 mg, 0.12 mmol) and 2-cyclopropyl-1,3-thiazole-4-carboxylic acid (21 mg, 0.12 mmol) were coupled in a similar fashion as for 74, to afford 20 mg (38%) of the title compound as an off-white solid: ¹H NMR (500 MHz, DMSO-d₆) δ 10.83 (s, 1H), 8.86 (s, 1H), 8.20 (s, 1H), 7.83-7.73 (m, 1H), 7.47 (t, J=2.0 Hz, 1H), 7.28 (t, J=7.9 Hz, 1H), 6.71 (dt, J=7.8, 1.3 Hz, 1H), 4.93 (d, J=6.0 Hz, 2H), 4.84 (d, J=6.0 Hz, 2H), 3.48 (s, 2H), 2.91 (s, 3H); LCMS: C₁₈H₁₆F₃N₅O₂S requires: 423, found: m/z=424 [M+H]⁺.

### Example 496: 6-cyclopropyl-4-{[(3S)-3-fluoropyrrolidin-1-yl]methyl}-N-(3-{3-[(4-methyl-1,2,4-triazol-3-yl)methyl]oxetan-3-yl}phenyl)pyridine-2-carboxamide

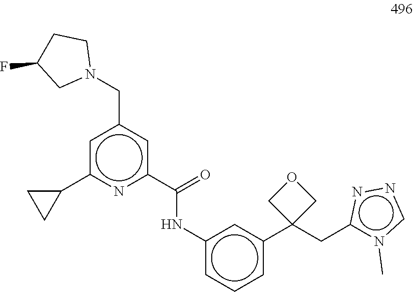

Step 1: Synthesis of methyl 4-(bromomethyl)-6-chloropicolinate. A mixture of methyl 6-chloro-4-methylpyridine-2-carboxylate (1.72 g, 9.25 mmol, 1.0 eq), NBS (1.65 g, 9.3 mmol, 1.0 eq), BPO (224 mg, 0.92 mmol, 0.1 eq) and trifluoromethyl benzene (34 mL) was heated at 80° C. for 28 h. The mixture was diluted with hexanes (50 mL), filtered, concentrated onto Celite and purified by chromatography over silica gel to afford 690 mg (28%) of the title compound.

Step 2: Synthesis of methyl (S)-6-chloro-4-((3-fluoropyrrolidin-1-yl)methyl)picolinate. A solution of methyl 4-(bromomethyl)-6-chloropyridine-2-carboxylate (690 mg, 2.6 mmol, 1.0 eq.), (3S)-3-fluoropyrrolidine hydrochloride (330 mg, 2.6 mmol, 1 eq.) DIPEA (1.4 mL, 7.8 mmol, 3 eq.), and MeCN (20 mL) was maintained at rt for 4h. The solution was concentrated onto Celite and purified by chromatography C to afford 550 mg (77%) of the title compound as a brown solid.

Step 3: Synthesis of methyl (S)-6-cyclopropyl-4-((3-fluoropyrrolidin-1-yl)methyl)picolinate. bromo(cyclopropyl)zinc (0.5M in THF, 10 mL, 5.0 mmol, 2.5 eq) was added to a cool (ice-water bath) solution of methyl 6-chloro-4-{[(3S)-3-fluoropyrrolidin-1-yl]methyl}pyridine-2-carboxylate (550 mg, 2.0 mmol, 1 eq.), 2′-(dicyclohexylphosphanyl)-N2,N2,N6,N6-tetramethyl-[1,1′-biphenyl]-2,6-diamine (176 mg, 0.40 mmol, 0.2 eq.), Pd(OAc)₂ (45 mg, 0.2 mmol, 0.1 eq.) and THF (10 mL). The cooling bath was removed upon completion of addition, and the solution maintained for 18 h. The solution was poured into sat. aq. NH₄Cl (50 mL), followed by General Work-up Procedure 1. Purification by chromatography A afforded 300 mg (53%) the title compound as a purple solid.

Step 4: Synthesis of (S)-6-cyclopropyl-4-((3-fluoropyrrolidin-1-yl)methyl)picolinic acid. A mixture of methyl 6-cyclopropyl-4-{[(3S)-3-fluoropyrrolidin-1-yl]methyl}pyridine-2-carboxylate (150 mg, 0.54 mmol) LiOH H₂O (68 mg, 1.62 mmol), THF (2 mL) and water (0.5 mL) was stirred vigorously for 4.5 h. Hydrochloric acid (2M, 60 μL.) was added and the THF removed under reduced pressure. The water was then removed by lyophilization to afford the title compound as a brown solid, which was carried on without purification.

Step 5: Synthesis of 6-cyclopropyl-4-{[(3S)-3-fluoropyrrolidin-1-yl]methyl}-N-(3-{3-[(4-methyl-1,2,4-triazol-3-yl)methyl]oxetan-3-yl}phenyl)pyridine-2-carboxamide. 3-{3-[(4-methyl-1,2,4-triazol-3-yl)methyl]oxetan-3-yl}aniline (132 mg, 0.54 mmol) and (S)-6-cyclopropyl-4-((3-fluoropyrrolidin-1-yl)methyl)picolinic acid (142 mg, 0.54 mmol) were coupled in a similar fashion as for 74, to afford the title compound as an off-white solid: ¹H NMR (500 MHz, DMSO-d₆) δ 10.12 (s, 1H), 8.20 (s, 1H), 7.83 (d, J=1.4 Hz, 1H), 7.73 (ddd, J=8.2, 2.1, 1.0 Hz, 1H), 7.47-7.37 (m, 2H), 7.27 (t, J=7.9 Hz, 1H), 6.69-6.61 (m, 1H), 5.34-5.13 (m, 1H), 4.94 (d, J=5.9 Hz, 2H), 4.86 (d, J=6.0 Hz, 2H), 3.78-3.62 (m, 2H), 3.49 (s, 2H), 2.92 (s, 3H), 2.86-2.75 (m, 2H), 2.66 (ddd, J=31.4, 11.4, 4.9 Hz, 1H), 2.41-2.33 (m, 1H), 2.28-2.21 (m, 1H), 2.21-2.09 (m, 1H), 1.98-1.84 (m, 1H), 1.20-1.10 (m, 2H), 1.08-0.99 (m, 2H); LCMS: C₂₇H₃₁FN₆O₂ requires: 490, found: m/z=491 [M+H]⁺.

### Example 497: 4-{2-azabicyclo[3.1.0]hexan-2-ylmethyl}-6-cyclopropyl-N-(3-{3-[(4-methyl-1,2,4-triazol-3-yl)methyl]oxetan-3-yl}phenyl)pyridine-2-carboxamide

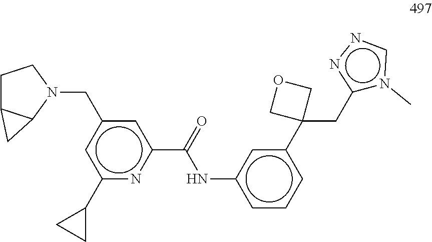

Step 1: Synthesis of methyl 6-cyclopropyl-4-formylpicolinate. A mixture of methyl 6-cyclopropyl-4-methylpyridine-2-carboxylate (1.2 g, 6.1 mmol), SeO₂ (880 mg, 7.9 mmol), acetic anhydride (2.4 mL, 25 mmol) and acetic acid (10 mL) was heated at reflux for 23 h, then cooled to rt. EtOAc (50 mL) was added, filtered through Celite, and concentrated to a film. The residue was dissolved in EtOAc and concentrated onto Celite and purified by chromatography over silica gel to afford 690 mg (55%) of the title compound.

Step 2: Synthesis of methyl 4-((2-azabicyclo[3.1.0]hexan-2-yl)methyl)-6-cyclopropylpicolinate. A mixture of methyl 6-cyclopropyl-4-formylpyridine-2-carboxylate (230 mg, 1.1 mmol), 2-azabicyclo[3.1.0]hexane hydrochloride (400 mg, 3.3 mmol), TEA (540 μL, 3.9 mmol) and DCM (3 mL) was sonicated for 1 minute. Sodium triacetoxyborohydride (490 mg, 2.3 mmol) was added and the mixture heated with stirring in a sealed vial at 45-50° C. for 3 h. The mixture was diluted with MeOH and EtOAc and concentrated onto Celite. Purification using Chromatography D afforded 170 mg (56%) of the title compound.

Step 3: Synthesis of 4-((2-azabicyclo[3.1.0]hexan-2-yl)methyl)-6-cyclopropylpicolinic acid. The title compound was prepared from methyl 4-((2-azabicyclo[3.1.0]hexan-2-yl)methyl)-6-cyclopropylpicolinate (170 mg, 0.62 mmol) according to the procedure of 496, Step 4.

Step 4: Synthesis of 4-{2-azabicyclo[3.1.0]hexan-2-ylmethyl}-6-cyclopropyl-N-(3-{3-[(4-methyl-1,2,4-triazol-3-yl)methyl]oxetan-3-yl}phenyl)pyridine-2-carboxamide. 3-{3-[(4-methyl-1,2,4-triazol-3-yl)methyl]oxetan-3-yl}aniline (152 mg, 0.62 mmol) and 4-{2-azabicyclo[3.1.0]hexan-2-ylmethyl}-6-cyclopropylpyridine-2-carboxylic acid (160 mg, 0.62 mmol) were coupled in a similar fashion as for 74, to afford the title compound as an off-white solid: ¹H NMR (500 MHz, DMSO-d₆) δ 10.12 (s, 1H), 8.20 (s, 1H), 7.86 (s, 1H), 7.74 (dd, J=8.2, 2.1 Hz, 1H), 7.46 (d, J=1.4 Hz, 1H), 7.41 (t, J=1.9 Hz, 1H), 7.27 (t, J=7.9 Hz, 1H), 6.66 (dt, J=7.9, 1.2 Hz, 1H), 4.94 (d, J=5.9 Hz, 2H), 4.86 (d, J=6.0 Hz, 2H), 3.73 (d, J=14.1 Hz, 1H), 3.62 (d, J=14.2 Hz, 1H), 3.49 (s, 2H), 2.92 (s, 3H), 2.83-2.74 (m, 1H), 2.60 (td, J=5.8, 2.6 Hz, 1H), 2.25 (tt, J=8.1, 4.9 Hz, 1H), 1.98-1.84 (m, 2H), 1.84-1.74 (m, 1H), 1.39 (ddd, J=10.3, 8.1, 4.3 Hz, 1H), 1.19-1.10 (m, 2H), 1.07-0.96 (m, 2H), 0.77-0.67 (m, 1H), 0.07 (dd, J=9.8, 4.1 Hz, 1H); LCMS: C₂₈H₃₂N₆O₂ requires: 484.6, found: m/z=485 [M+H]⁺.

### Example 498: 4-{3-azabicyclo[3.1.0]hexan-3-ylmethyl}-6-cyclopropyl-N-(3-{3-[(4-methyl-1,2,4-triazol-3-yl)methyl]oxetan-3-yl}phenyl)pyridine-2-carboxamide

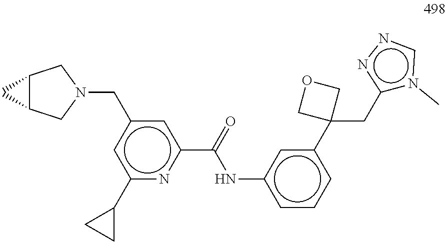

Step 1: Synthesis of methyl 4-{3-azabicyclo[3.1.0]hexan-3-ylmethyl}-6-cyclo-propylpyridine-2-carboxylate. The title compound (253 mg, 84%) was prepared from methyl 6-cyclopropyl-4-formylpyridine-2-carboxylate (226 mg, 1.1 mmol) and 3-azabicyclo[3.1.0]-hexane hydrochloride (395 mg, 3.3 mmol) according the procedure of 497, Step 2.

Step 2: Synthesis of 4-{3-azabicyclo[3.1.0]hexan-3-ylmethyl}-6-cyclopropyl-pyridine-2-carboxylic acid. The title compound was prepared from methyl 4-{3-azabicyclo[3.1.0]hexan-3-ylmethyl}-6-cyclopropylpyridine-2-carboxylate (253 mg, 0.93 mmol) according to the procedure of 496, Step 4.

Step 3: Synthesis of 4-{3-azabicyclo[3.1.0]hexan-3-ylmethyl}-6-cyclopropyl-N-(3-{3-[(4-methyl-1,2,4-triazol-3-yl)methyl]oxetan-3-yl}phenyl)pyridine-2-carboxamide. 3-{3-[(4-methyl-1,2,4-triazol-3-yl)methyl]oxetan-3-yl}aniline (227 mg, 0.93 mmol) and 4-{3-azabicyclo[3.1.0]hexan-3-ylmethyl}-6-cyclopropylpyridine-2-carboxylic acid (240 mg, 0.93 mmol) were coupled in a similar fashion as for 74, to afford 172 mg (38%) of the title compound as an off-white solid: ¹H NMR (500 MHz, DMSO-d₆) δ 10.11 (s, 1H), 8.19 (s, 1H), 7.76 (d, J=1.4 Hz, 1H), 7.72 (dd, J=8.2, 1.7 Hz, 1H), 7.42 (t, J=1.9 Hz, 1H), 7.36 (d, J=1.4 Hz, 1H), 7.27 (t, J=7.9 Hz, 1H), 6.65 (dt, J=8.0, 1.2 Hz, 1H), 4.94 (d, J=5.9 Hz, 2H), 4.86 (d, J=6.0 Hz, 2H), 3.66 (s, 2H), 3.49 (s, 2H), 2.92 (s, 3H), 2.88 (d, J=8.5 Hz, 2H), 2.38-2.29 (m, 2H), 2.23 (tt, J=8.1, 4.8 Hz, 1H), 1.43-1.33 (m, 2H), 1.13 (dt, J=5.9, 3.1 Hz, 2H), 1.03 (dt, J=8.2, 3.1 Hz, 2H), 0.71 (q, J=3.8 Hz, 1H), 0.36 (td, J=7.7, 3.9 Hz, 1H); LCMS: C₂₈H₃₂N₆O₂ requires: 484, found: m/z=485 [M+H]⁺.

### Example 499: 1-(6-cyclopropyl-4-((6,6-difluoro-3-azabicyclo[3.1.0]hexan-3-yl)methyl)-pyridin-2-yl)-2-(3-(3-((4-methyl-4H-1,2,4-triazol-3-yl)methyl)oxetan-3-yl)phenyl)ethan-1-one

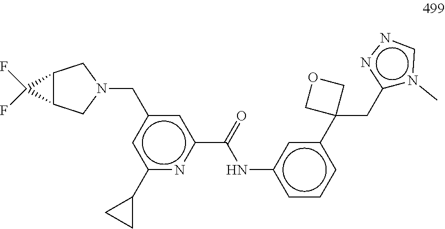

Step 1: Synthesis of methyl 6-cyclopropyl-4-({6,6-difluoro-3-azabicyclo[3.1.0]hexan-3-yl}methyl)pyridine-2-carboxylate. The title compound (249 mg, 73%) was prepared from methyl 6-cyclopropyl-4-formylpyridine-2-carboxylate (226 mg, 1.1 mmol) and 6,6-difluoro-3-azabicyclo[3.1.0]hexane hydrochloride (514 mg, 3.3 mmol) according the procedure of 497, Step 2.

Step 2: Synthesis of 6-cyclopropyl-4-({6,6-difluoro-3-azabicyclo[3.1.0]hexan-3-yl}methyl)pyridine-2-carboxylic acid. The title compound was prepared from methyl 6-cyclopropyl-4-({6,6-difluoro-3-azabicyclo[3.1.0]hexan-3-yl}methyl)pyridine-2-carboxylate (249 mg, 0.81 mmol) according to the procedure of 496, Step 4.

Step 3: Synthesis of l-(6-cyclopropyl-4-((6,6-difluoro-3-azabicyclo[3.1.0]hexan-3-yl)methyl)pyridin-2-yl)-2-(3-(3-((4-methyl-4H-1,2,4-triazol-3-yl)methyl)oxetan-3-yl)phenyl)ethan-1-one. 3-{3-[(4-methyl-1,2,4-triazol-3-yl)methyl]oxetan-3-yl}aniline (198 mg, 0.81 mmol) and 6-cyclopropyl-4-({6,6-difluoro-3-azabicyclo[3.1.0]hexan-3-yl}methyl)pyridine-2-carboxylic acid (238 mg, 0.81 mmol) were coupled in a similar fashion as for 74, to afford 153 mg (36%) of the title compound as an off-white solid: ¹H NMR (500 MHz, DMSO-d₆) δ 10.12 (s, 1H), 8.19 (s, 1H), 7.80 (d, J=1.4 Hz, 1H), 7.73 (dd, J=8.1, 2.1 Hz, 1H), 7.42 (t, J=1.9 Hz, 1H), 7.37 (d, J=1.3 Hz, 1H), 7.27 (t, J=7.9 Hz, 1H), 6.65 (dt, J=7.7, 1.3 Hz, 1H), 4.94 (d, J=6.0 Hz, 2H), 4.86 (d, J=6.0 Hz, 2H), 3.71 (s, 2H), 3.49 (s, 2H), 2.99 (dd, J=9.8, 2.6 Hz, 2H), 2.92 (s, 3H), 2.82 (d, J=9.5 Hz, 2H), 2.36 (dt, J=13.5, 2.1 Hz, 3H), 2.22 (tt, J=8.1, 4.8 Hz, 1H), 1.17-1.08 (m, 2H), 1.08-0.97 (m, 2H); LCMS: C₂₈H₃₀N₆O₂ requires: 520, found: m/z=521 [M+H]⁺.

### Example 500: 4-{5-azaspiro[2.4]heptan-5-ylmethyl}-6-cyclopropyl-N-(3-{3-[(4-methyl-1,2-oxazol-3-yl)methyl]oxetan-3-yl}phenyl)pyridine-2-carboxamide

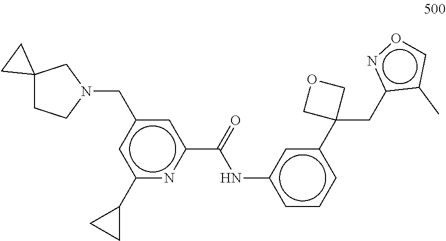

Step 1: Synthesis of ethyl 4-{5-azaspiro[2.4]heptan-5-ylmethyl}-6-cyclopropyl-pyridine-2-carboxylate. The title compound (284 mg, 93%) was prepared from ethyl 6-cyclo-propyl-4-formylpyridine-2-carboxylate (200 mg, 0.91 mmol) and 5-azaspiro[2.4]heptane hydrochloride (250 mg, 1.9 mmol) according the procedure of 497, Step 2.

Step 2: Synthesis of 4-{5-azaspiro[2.4]heptan-5-ylmethyl}-6-cyclopropylpyridine-2-carboxylic acid. Prepared according to the procedure from 496, Step 2 using 5-azaspiro[2.4]-heptane hydrochloride (60 mg, 0.20 mmol).

Step 3: Synthesis of 4-{5-azaspiro[2.4]heptan-5-ylmethyl}-6-cyclopropyl-N-(3-{3-[(4-methyl-1,2-oxazol-3-yl)methyl]oxetan-3-yl}phenyl)pyridine-2-carboxamide. 4-{5-azaspiro[2.4]heptan-5-ylmethyl}-6-cyclopropylpyridine-2-carboxylic acid (54 mg, 0.2 mmol) and 3-{3-[(4-methyl-1,2-oxazol-3-yl)methyl]oxetan-3-yl}aniline (49 mg, 0.20 mmol) were coupled using the procedure from 74 to afford 38 mg (39%) of the title compound as an off-white solid: ¹H NMR (500 MHz, DMSO-d₆) δ 10.12 (s, 1H), 8.42 (s, 1H), 7.83 (d, J=1.3 Hz, 1H), 7.72 (dd, J=8.2, 2.0 Hz, 1H), 7.47 (t, J=1.9 Hz, 1H), 7.44 (d, J=1.3 Hz, 1H), 7.29 (t, J=7.9 Hz, 1H), 6.78-6.68 (m, 1H), 4.91-4.78 (m, 4H), 3.67 (s, 2H), 2.68 (t, J=6.8 Hz, 2H), 2.25 (tt, J=8.4, 4.8 Hz, 1H), 1.77 (t, J=6.8 Hz, 2H), 1.30 (d, J=1.1 Hz, 3H), 1.14 (dt, J=6.0, 3.1 Hz, 2H), 1.03 (dt, J=8.2, 3.2 Hz, 2H), 0.51 (dt, J=8.5, 1.9 Hz, 4H); LCMS: C₃₀H₃₄N₄O₃ requires: 498, found: m/z=499 [M+H]⁺.

### Example 501: 6-cyclopropyl-N-(3-{3-[(4-methyl-1,2,4-triazol-3-yl)methyl]oxetan-3-yl}phenyl)-4-{[(2R)-2-methylmorpholin-4-yl]methyl}pyridine-2-carboxamide

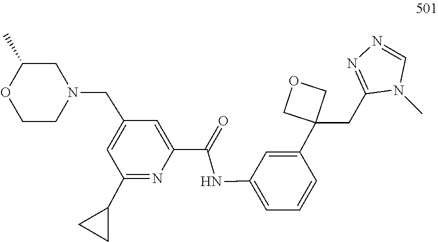

Step 1: Synthesis of ethyl 6-cyclopropyl-4-{[(2R)-2-methylmorpholin-4-yl]methyl}pyridine-2-carboxylate. The title compound (452 mg, 93%) was prepared from ethyl 6-cyclopropyl-4-formylpyridine-2-carboxylate (500 mg, 0.91 mmol) and (2R)-2-methylmorpholine (460 mg, 1.9 mmol) according the procedure of Example 500 Step 1.

Step 2: Synthesis of (R)-6-cyclopropyl-4-((2-methylmorpholino)methyl)picolinic acid. Prepared according to the procedure 496, Step 2 using 5-azaspiro[2.4]heptane hydrochloride (60 mg, 0.20 mmol).

Step 3: Synthesis of 6-cyclopropyl-N-(3-{3-[(4-methyl-1,2,4-triazol-3-yl)methyl]oxetan-3-yl}phenyl)-4-{[(2R)-2-methylmorpholin-4-yl]methyl}pyridine-2-carboxamide. 3-{3-[(4-methyl-1,2,4-triazol-3-yl)methyl]oxetan-3-yl}aniline (105 mg, 0.43 mmol) and 6-cyclopropyl-4-{[(2R)-2-methylmorpholin-4-yl]methyl}pyridine-2-carboxylate (120 mg, 0.43 mmol) were coupled using procedure 74 to afford 160 mg (71%) of the title compound as an off-white solid: ¹H NMR (500 MHz, DMSO-d₆) δ 10.11 (d, J=2.0 Hz, 1H), 8.19 (d, J=1.9 Hz, 1H), 7.83 (s, 1H), 7.72 (d, J=8.1 Hz, 1H), 7.47-7.38 (m, 2H), 7.27 (td, J=8.0, 1.9 Hz, 1H), 6.66 (d, J=7.7 Hz, 1H), 4.94 (dd, J=6.1, 1.9 Hz, 2H), 4.86 (dd, J=6.1, 1.9 Hz, 2H), 3.74 (d, J=11.4 Hz, 1H), 3.59-3.52 (m, 3H), 3.49 (d, J=2.0 Hz, 2H), 2.92 (d, J=1.9 Hz, 3H), 2.67 (dd, J=11.3, 2.2 Hz, 1H), 2.60 (d, J=11.4 Hz, 1H), 2.24 (ddt, J=11.6, 8.8, 4.5 Hz, 1H), 2.12-2.03 (m, 2H), 1.86-1.68 (m, 1H), 1.19-1.11 (m, 2H), 1.07-0.98 (m, 5H); LCMS: C₃₀H₃₄N₆O₃ requires: 502, found: m/z=503 [M+H]⁺.

### Example 502: 6-cyclopropyl-4-(difluoromethoxy)-N-(3-{3-[(4-methyl-1,2,4-triazol-3-yl)methyl]oxetan-3-yl}phenyl)pyridine-2-carboxamide

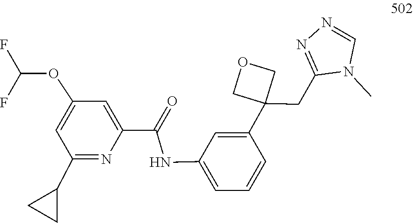 [SMILES: Cn1cnnc1CC1(c2cccc(NC(=O)c3cc(OC(F)F)cc(C4CC4)n3)c2)COC1]

Step 1: Synthesis of methyl 6-cyclopropyl-4-[(4-methoxyphenyl)methoxy]-pyridine-2-carboxylate: To a solution of (4-methoxyphenyl)methanol (3.2 g, 23.46 mmol) in DMF (40.0 mL) was added NaH (1.3 g, 32.00 mmol, 60%) in portions at 0° C. The resulting mixture was stirred for 30 min at 0° C., and then a solution of methyl 4-chloro-6-cyclopropylpyridine-2-carboxylate (4.5 g, 21.33 mmol) in DMF (30.0 mL) was added to the above mixture dropwise at 0° C. The mixture was stirred at rt for 16 h. The mixture was poured into saturated NH₄Cl aqueous solution followed by General Work-up Procedure 1. The residue was purified by chromatography A to the title compound (2.6 g, 70% purity, 27%) as a colorless syrup, which was used in the next step without purification.

Step 2: Synthesis of methyl 6-cyclopropyl-4-hydroxypyridine-2-carboxylate: To a mixture of methyl 6-cyclopropyl-4-[(4-methoxyphenyl)methoxy]pyridine-2-carboxylate (2.6 g, 5.81 mmol, 70% purity) in dichloromethane (30.0 mL) was added trifluoracetic acid (5.0 mL). The mixture was stirred at room temperature for 2 h. The resulting mixture was concentrated under vacuum. The residue was purified by chromatography C to afford the title compound (800 mg, 71%) as a colorless solid.

Step 3: Synthesis of methyl 6-cyclopropyl-4-(difluoromethoxy)pyridine-2-carboxylate: A mixture of methyl 6-cyclopropyl-4-hydroxypyridine-2-carboxylate (800.0 mg, 4.12 mmol), ethyl 2-chloro-2,2-difluoroacetate (1.3 g, 8.24 mmol) and Cs₂CO₃ (2.8 g, 8.24 mmol) in butan-2-one (25 mL) was stirred at 80° C. for 16 h under N₂ atmosphere. The solids were filtered off and the filtrate was concentrated under vacuum. The residue was purified by chromatography A to afford the title compound (400.0 mg, 40%) as a white solid.

Step 5: Synthesis of 6-cyclopropyl-4-(difluoromethoxy)picolinic acid: A mixture of methyl 6-cyclopropyl-4-(difluoromethoxy)pyridine-2-carboxylate (400.0 mg, 1.65 mmol), THF/water (12 mL, v/v=l/l) and LiOH (78.7 mg, 3.28 mmol) was stirred at rt for 3 h. The organic solvent was removed by evaporation. The residue was diluted with water and acidified to pH 3 by HCl (3 N). The solids were collected by filtration and further purified by chromatography C to afford the title compound (177.5 mg, 47%) as a colorless solid.

Step 6: Synthesis of 6-cyclopropyl-4-(difluoromethoxy)-N-(3-{3-[(4-methyl-1,2,4-triazol-3-yl)methyl]oxetan-3-yl}phenyl)pyridine-2-carboxamide. 3-{3-[(4-methyl-1,2,4-triazol-3-yl)methyl]oxetan-3-yl}aniline (53 mg, 0.22 mmol) and 6-cyclopropyl-4-(difluoromethoxy)picolinic acid (50 mg, 0.22 mmol) were coupled using procedure from 74 to afford 12 mg (12%) of the title compound as an off-white solid: ¹H NMR (500 MHz, DMSO-d₆) δ 10.17 (s, 1H), 8.20 (s, 1H), 7.72 (dd, J=7.6, 1.8 Hz, 1H), 7.60 (s, 1H), 7.59 (t, J=72.6 Hz, 1H), 7.44 (t, J=1.9 Hz, 1H), 7.32 (d, J=2.3 Hz, 1H), 7.28 (t, J=7.9 Hz, 1H), 6.68 (dt, J=7.7, 1.3 Hz, 1H), 4.94 (d, J=6.0 Hz, 2H), 4.86 (d, J=6.0 Hz, 2H), 3.49 (s, 2H), 2.93 (s, 3H), 2.27 (tt, J=8.0, 4.8 Hz, 1H), 1.20 (dt, J=6.1, 3.2 Hz, 2H), 1.13-1.00 (m, 2H); LCMS: C₂₃H₂₃N₅O₃ requires: 455, found: m/z=456 [M+H]⁺.

### Example 503: 2-cyclopropyl-6-[(dimethylamino)methyl]-N-(3-{3-[(4-methyl-1,2,4-triazol-3-yl)methyl]oxetan-3-yl}phenyl)pyrimidine-4-carboxamide

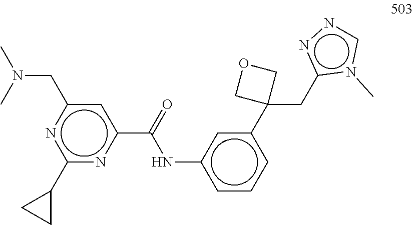

Step 1: Synthesis of ethyl 2-cyclopropyl-6-methylpyrimidine-4-carboxylate. To a solution of ethyl 2,4-dioxopentanoate (10.0 g, 63.29 mmol) and cyclopropanecarboximidamide (6.9 g, 57.5 mmol) in DMF (200 mL) was added pTsOH.H₂O (1.1 g, 5.8 mmol). The solution was stirred at 100° C. for 72 h. The mixture was cooled to rt and diluted with water, followed by General Work-up Procedure 1. The residue was purified by chromatography A to afford the title compound (4.8 g, 40%) as a light-yellow oil.

Step 2: Synthesis of ethyl 6-(bromomethyl)-2-cyclopropylpyrimidine-4-carboxylate. To a solution of ethyl 2-cyclopropyl-6-methylpyrimidine-4-carboxylate (5.0 g, 24.2 mmol) in HOAc (50 mL) was added bromine (3.86 g, 24.2 mmol). The solution was stirred at 80° C. for 1 h. The solvent was removed under vacuum. The residue was purified by chromatography A to afford the title compound (2.0 g, 30%) as a red solid.

Step 3: Synthesis of ethyl 2-cyclopropyl-6-[(dimethylamino)methyl]pyrimidine-4-carboxylate. A solution of ethyl 6-(bromomethyl)-2-cyclopropylpyrimidine-4-carboxylate (50 mg, 0.18 mmol), dimethylamine hydrochloride (16 mg, 0.18 mmol), DIPEA (61 μL, 0.35 mmol) and MeCN (0.5 mL) was maintained at rt for 2h. The solution was concentrated to an oil under reduced pressure and carried on without purification.

Step 4: Synthesis of 2-cyclopropyl-6-[(dimethylamino)methyl]pyrimidine-4-carboxylic acid. The title compound was prepared from ethyl 2-cyclopropyl-6-[(dimethylamino)methyl]pyrimidine-4-carboxylate (45 mg, 0.18 mmol.) according to the procedure of 496, Step 4.

Step 5: Synthesis of 2-cyclopropyl-6-[(dimethylamino)methyl]-N-(3-{3-[(4-methyl-1,2,4-triazol-3-yl)methyl]oxetan-3-yl}phenyl)pyrimidine-4-carboxamide. 3-{3-[(4-methyl-1,2,4-triazol-3-yl)methyl]oxetan-3-yl}aniline (44 mg, 0.18 mmol) and 2-cyclopropyl-6-[(dimethylamino)methyl]pyrimidine-4-carboxylic acid (40 mg, 0.18 mmol, 1 eq) were coupled in a similar fashion as for 74, to afford the title compound as an off-white solid: ¹H NMR (500 MHz, Acetonitrile-d₃) δ 9.95 (s, 1H), 7.98 (s, 1H), 7.91 (s, 1H), 7.75 (dd, J=8.2, 2.0 Hz, 1H), 7.36-7.26 (m, 2H), 6.73 (dd, J=7.8, 1.6 Hz, 1H), 4.99 (d, J=6.0 Hz, 2H), 4.94 (d, J=6.0 Hz, 2H), 3.50 (s, 2H), 2.89 (s, 3H), 2.55 (s, 6H), 2.35 (tt, J=8.2, 4.4 Hz, 1H), 1.01-0.88 (m, 4H); LCMS: C₂₄H₂₉N₇O₂ requires: 447, found: m/z=448 [M+H]⁺.

### Example 504: 6-(azetidin-1-ylmethyl)-2-cyclopropyl-N-(3-{3-[(4-methyl-1,2,4-triazol-3-yl)methyl]oxetan-3-yl}phenyl)pyrimidine-4-carboxamide

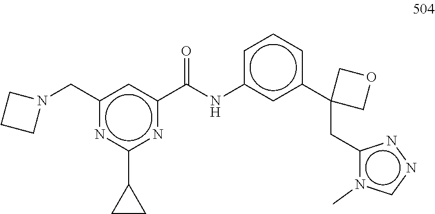

Followed Example 503 using azetidine in Step 3 to afford compound 504 (6 mg). ¹H NMR (500 MHz, DMSO-d₆) δ 10.44 (s, 1H), 8.20 (s, 1H), 7.85-7.69 (m, 2H), 7.47 (t, J=1.9 Hz, 1H), 7.29 (t, J=7.9 Hz, 1H), 6.71 (d, J=7.9 Hz, 1H), 4.94 (d, J=6.0 Hz, 2H), 4.86 (d, J=6.0 Hz, 2H), 2.94 (s, 3H), 2.34 (d, J=8.5 Hz, 3H), 0.97-0.87 (m, 4H); LCMS: C₂₅H₂₉N₇O₂ requires: 459, found: m/z=460 [M+H]⁺.

### Example 505: 2-cyclopropyl-6-{[(3S)-3-hydroxypyrrolidin-1-yl]methyl}-N-(3-{3-[(4-methyl-1,2,4-triazol-3-yl)methyl]oxetan-3-yl}phenyl)pyrimidine-4-carboxamide

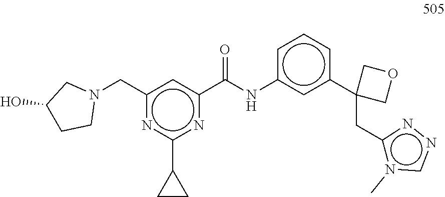

Followed example 503 using (3S)-pyrrolidin-3-ol in Step 3 to afford compound 505 (11 mg). ¹H NMR (500 MHz, Acetonitrile-d₃) δ 9.97 (s, 1H), 7.99 (s, 1H), 7.91 (s, 1H), 7.73 (dd, J=8.1, 2.0 Hz, 1H), 7.34 (d, J=2.0 Hz, 1H), 7.30 (t, J=7.9 Hz, 1H), 6.73 (dd, J=7.6, 1.6 Hz, 1H), 4.98 (d, J=6.0 Hz, 2H), 4.94 (d, J=6.1 Hz, 2H), 4.40 (s, 1H), 4.19 (s, 2H), 3.50 (s, 2H), 2.89 (s, 3H), 2.35 (tt, J=8.3, 4.5 Hz, 1H), 1.89 (s, 1H), 0.96-0.85 (m, 4H); LCMS: C₂₆H₃₁N₇O₃ requires: 489, found: m/z=490 [M+H]⁺.

### Example 506: 2-cyclopropyl-N-(3-{3-[(4-methyl-1,2,4-triazol-3-yl)methyl]oxetan-3-yl}phenyl)-6-(1,2,3-triazol-1-ylmethyl)pyrimidine-4-carboxamide

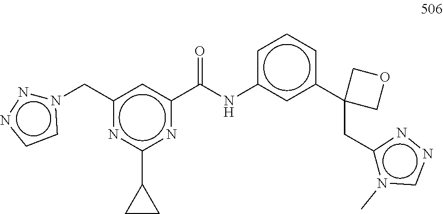

Followed Example 503 using 1,2,3-triazole in Step 3 (127 mg). ¹H NMR (500 MHz, DMSO-d₆) δ 10.46 (s, 1H), 8.28 (d, J=1.0 Hz, 1H), 8.19 (s, 1H), 7.85 (d, J=1.0 Hz, 1H), 7.80-7.72 (m, 1H), 7.50-7.41 (m, 2H), 7.28 (t, J=7.9 Hz, 1H), 6.69 (dt, J=7.8, 1.3 Hz, 1H), 5.87 (s, 2H), 4.93 (d, J=6.0 Hz, 2H), 4.85 (d, J=6.0 Hz, 2H), 3.48 (s, 2H), 2.92 (s, 3H), 2.38-2.30 (m, 1H), 1.15-1.07 (m, 4H); LCMS: C₂₄H₂₅N₉O₂ requires: 471, found: m/z=472 [M+H]⁺.

### Example 507: 2-cyclopropyl-6-{[(3S)-3-hydroxypyrrolidin-1-yl]methyl}-N-(3-{3-[(4-methyl-1,2-oxazol-3-yl)methyl]oxetan-3-yl}phenyl)pyrimidine-4-carboxamide

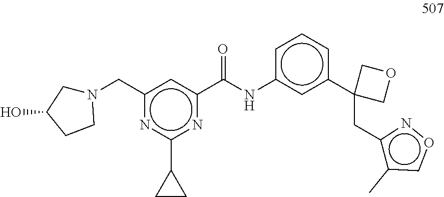

Synthesized according to the procedure of 503 using 3-{3-[(4-methyl-1,2-oxazol-3-yl)methyl]oxetan-3-yl}aniline (49 mg, 0.2 mmol, 1 eq.) and 2-cyclopropyl-6-{[(3S)-3-hydroxypyrrolidin-1-yl]methyl}pyrimidine-4-carboxylic acid (53 mg, 0.2 mmol, 1 eq.) to afford the title compound (15 mg) as an off-white solid: ¹H NMR (500 MHz, DMSO-d₆) δ 10.41 (s, 1H), 8.42 (s, 1H), 7.84 (s, 1H), 7.78 (dd, J=7.9, 2.0 Hz, 1H), 7.53 (t, J=1.9 Hz, 1H), 7.30 (t, J=7.9 Hz, 1H), 6.77 (d, J=7.7 Hz, 1H), 4.90-4.80 (m, 4H), 4.75 (d, J=4.4 Hz, 1H), 4.28-4.19 (m, 1H), 3.79-3.67 (m, 2H), 3.36 (s, 2H), 2.76 (dd, J=9.7, 6.1 Hz, 1H), 2.69 (q, J=7.7 Hz, 1H), 2.42 (dd, J=9.7, 3.6 Hz, 1H), 2.03 (dq, J=14.2, 7.4 Hz, 1H), 1.65-1.55 (m, 1H), 1.30 (s, 3H), 1.17 (dt, J=5.5, 3.0 Hz, 2H), 1.12 (dt, J=8.4, 3.2 Hz, 2H); LCMS: C₂₇H₃₁N₅O₄ requires: 489, found: m/z=490 [M+H]⁺.

### Example 508: 6-{5-azaspiro[2.4]heptan-5-ylmethyl}-2-cyclopropyl-N-(3-{3-[(4-methyl-1,2-oxazol-3-yl)methyl]oxetan-3-yl}phenyl)pyrimidine-4-carboxamide

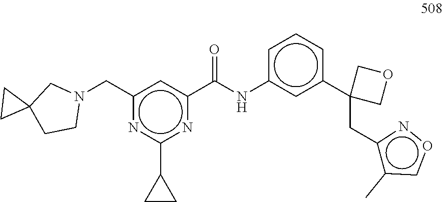

Step 1: Synthesis of 6-{5-azaspiro[2.4]heptan-5-ylmethyl}-2-cyclopropyl-pyrimidine-4-carboxylic acid. Followed Example 503 using 5-azaspiro[2.4]heptane hydrochloride in Step 3 to afford the title compound (60 mg).

Step 2: Synthesis of 6-{5-azaspiro[2.4]heptan-5-ylmethyl}-2-cyclopropyl-N-(3-{3-[(4-methyl-1,2-oxazol-3-yl)methyl]oxetan-3-yl}phenyl)pyrimidine-4-carboxamide. 3-{3-[(4-methyl-1,2-oxazol-3-yl)methyl]oxetan-3-yl}aniline (49 mg, 0.2 mmol) and 6-{5-azaspiro[2.4]-heptan-5-ylmethyl}-2-cyclopropylpyrimidine-4-carboxylic acid (55 mg, 0.2 mmol) were coupled according to the procedure of 74 to afford 12 mg (12%) of the title compound as an off-white solid: ¹H NMR (500 MHz, DMSO-d₆) δ 10.41 (s, 1H), 8.47-8.40 (m, 1H), 7.86 (s, 1H), 7.78 (dd, J=8.2, 2.0 Hz, 1H), 7.53 (d, J=2.0 Hz, 1H), 7.30 (t, J=7.9 Hz, 1H), 6.77 (d, J=7.8 Hz, 1H), 4.84 (d, J=1.3 Hz, 4H), 3.75 (s, 2H), 3.36 (s, 2H), 2.75 (t, J=6.8 Hz, 2H), 2.34 (dt, J=8.3, 4.6 Hz, 2H), 1.78 (t, J=6.8 Hz, 2H), 1.30 (d, J=1.2 Hz, 3H), 1.17 (dt, J=5.4, 3.0 Hz, 2H), 1.11 (dt, J=8.2, 3.1 Hz, 2H), 0.57-0.46 (m, 4H); LCMS: C₂₉H₃₃N₅O₃ requires: 499, found: m/z=500 [M+H]⁺.

### Example 509: 2-cyclopropyl-6-methyl-N-(3-{3-[(4-methyl-1,2-oxazol-3-yl)methyl]oxetan-3-yl}phenyl)pyrimidine-4-carboxamide

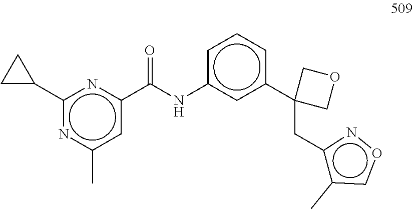

3-{3-[(4-methyl-1,2-oxazol-3-yl)methyl]oxetan-3-yl}aniline (28 mg, 0.11 mmol) and 2-cyclopropyl-6-methylpyrimidine-4-carboxylic acid (26 mg, 0.15 mmol) were coupled in a similar fashion as for 74, to afford 31 mg (67%) of the title compound as an off-white solid: ¹H NMR (500 MHz, DMSO-d₆) δ 10.39 (s, 1H), 8.42 (d, J=1.3 Hz, 1H), 7.79 (dd, J=8.0, 2.0 Hz, 1H), 7.71 (s, 1H), 7.51 (t, J=1.9 Hz, 1H), 7.30 (t, J=8.0 Hz, 1H), 6.77 (dt, J=7.6, 1.3 Hz, 1H), 4.87-4.81 (m, 4H), 3.36 (s, 2H), 2.52 (s, 3H), 2.33 (ddd, J=9.4, 6.5, 4.0 Hz, 1H), 1.30 (s, 3H), 1.20-1.06 (m, 4H); LCMS: C₂₃H₂₄N₄O₃ requires: 404, found: m/z=405 [M+H]⁺.

### Example 510: 2-cyclopropyl-6-methyl-N-(3-{3-[(4-methyl-1H-pyrazol-3-yl)methyl]oxetan-3-yl}phenyl)pyrimidine-4-carboxamide

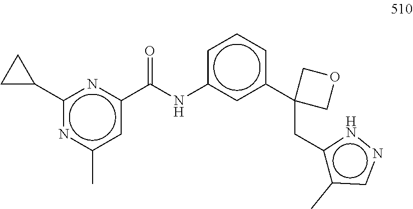

3-{3-[(4-methyl-1H-pyrazol-3-yl)methyl]oxetan-3-yl}aniline (30 mg, 0.12 mmol) and 2-cyclopropyl-6-methylpyrimidine-4-carboxylic acid (26 mg, 0.15 mmol) were coupled in a similar fashion as for 74, to afford 37 mg (74%) of the title compound as an off-white solid: ¹H NMR (500 MHz, Acetone-d₆) δ 11.49 (s, 1H), 10.16 (s, 1H), 7.83 (ddd, J=8.1, 2.2, 1.0 Hz, 1H), 7.76 (s, 1H), 7.42 (s, 1H), 7.27 (t, J=7.9 Hz, 1H), 7.19 (s, 1H), 6.70-6.65 (m, 1H), 4.94 (d, J=5.6 Hz, 2H), 4.88 (d, J=5.6 Hz, 2H), 3.31 (s, 2H), 2.55 (s, 3H), 2.29 (tt, J=8.1, 4.7 Hz, 1H), 1.38 (s, 3H), 1.19-1.13 (m, 2H), 1.13-1.04 (m, 2H); LCMS: C₂₃H₂₅N₅O₂ requires: 403, found: m/z=404 [M+H]⁺.

### Example 511: 2-cyclopropyl-6-methyl-N-{3-[2-methyl-1-(4-methyl-1,2,4-triazol-3-yl)propan-2-yl]phenyl}pyrimidine-4-carboxamide

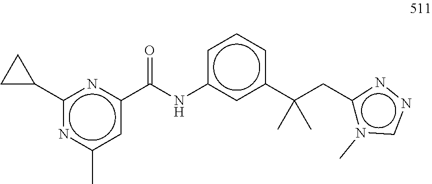

3-[2-methyl-1-(4-methyl-1,2,4-triazol-3-yl)propan-2-yl]aniline (32 mg, 0.14 mmol) and 2-cyclopropyl-6-methylpyrimidine-4-carboxylic acid (25 mg, 0.14 mmol) were coupled in a similar fashion as for 74, to afford 46 mg (84%) of the title compound as an off-white solid: ¹H NMR (500 MHz, DMSO-d₆) δ 10.39 (s, 1H), 8.22 (s, 1H), 7.82-7.74 (m, 2H), 7.71 (s, 1H), 7.28 (t, J=7.8 Hz, 1H), 7.11-7.02 (m, 1H), 3.14 (s, 3H), 2.98 (s, 2H), 2.38-2.30 (m, 1H), 1.43 (s, 6H), 1.17 (dt, J=5.6, 2.9 Hz, 2H), 1.10 (dq, J=10.3, 4.1, 3.6 Hz, 2H); LCMS: C₂₂H₂₆N₆O requires: 390, found: m/z=391 [M+H]⁺.

### Example 512: 2-cyclopropyl-6-{[(3S)-3-fluoropyrrolidin-1-yl]methyl}-N-{3-[(2R)-1,1,2-trifluoro-1-(4-methyl-1,2,4-triazol-3-yl)propan-2-yl]phenyl}pyrimidine-4-carboxamide

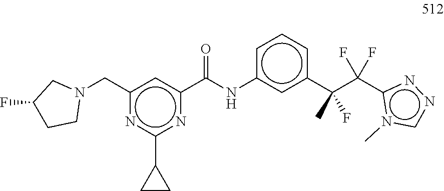

3-[(2R)-1,1,2-trifluoro-1-(4-methyl-1,2,4-triazol-3-yl) propan-2-yl]aniline (111 mg, 0.41 mmol) and 2-cyclopropyl-6-{[(3S)-3-fluoropyrrolidin-1-yl]methyl}pyrimidine-4-carboxylic acid (109 mg, 0.41 mmol) were coupled in a similar fashion as for 74, to afford 105 mg (50%) of the title compound as an off-white solid: 1H NMR (500 MHz, DMSO-d₆) δ 10.60 (s, 1H), 8.63 (s, 1H), 8.05-7.91 (m, 2H), 7.85 (s, 1H), 7.44 (t, J=8.3 Hz, 1H), 7.12 (d, J=7.9 Hz, 1H), 5.24 (d, J=55.6 Hz, 1H), 3.81 (s, 2H), 3.45 (s, 3H), 3.02-2.82 (m, 2H), 2.82-2.67 (m, 1H), 2.37 (tt, J=8.3, 4.7 Hz, 1H), 2.19 (ddd, J=28.0, 14.0, 7.1 Hz, 1H), 1.96 (d, J=24.2 Hz, 4H), 1.29-1.04 (m, 4H); LCMS: C₂₅H₂₇F₄N₇O requires: 517, found: m/z=518 [M+H]⁺.

### Example 513: 6-cyclopropoxy-2-(3-{3-[(4-methyl-1,2,4-triazol-3-yl)methyl]oxetan-3-yl}-phenyl)-4-(trifluoromethyl)-3H-isoindol-1-one

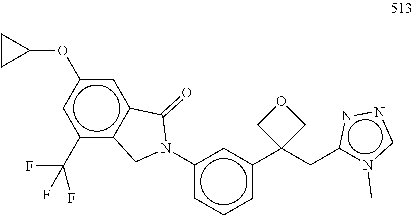

Step 1: Synthesis of methyl 2-methyl-3-(trifluoromethyl)-5-(vinyloxy)benzoate. A mixture of methyl 5-hydroxy-2-methyl-3-(trifluoromethyl)benzoate (1.0 g, 4.27 mmol), CU(OAC)₂ (775.6 mg, 4.27 mmol), pyridine (3.4 g, 42.73 mmol) and triethenyl-1,3,5,2,4,6-trioxatriborinane pyridine complex (1.03 g, 4.27 mmol) in DCM (24.0 mL) was stirred at rt for 16 h under O₂ atmosphere. The reaction was then quenched by the addition of saturated aqueous NH₄OAC and extracted with dichloromethane followed by General Work-up Procedure 1. The residue was purified by chromatography A to afford the title compound (774 mg, 69%) as a colorless oil.

Step 2: Synthesis of methyl 5-cyclopropoxy-2-methyl-3-(trifluoromethyl)benzoate. To a degassed solution of diethylzinc (5.2 mL, 1M in Et₂O) in DCM (5.0 mL, 78.7 mmol) was added TFA (569 mg, 5.81 mmol) dropwise below −5° C. After stirring for 10 min at −5° C., diiodomethane (1.43 g, 5.0 mmol) was added dropwise below −5° C. and stirred for another 10 min at −5° C. A solution of methyl 5-(ethenyloxy)-2-methyl-3-(trifluoromethyl)benzoate (500.0 mg, 1.92 mmol) in DCM (5.0 mL) added to the above mixture dropwise below −5° C. The mixture was allowed to warm up to rt and stirred for 40 min. The reaction was quenched by addition of saturated NH₄Cl aqueous solution and extracted with dichloromethane followed by General Work-up Procedure 1. The residue was purified by chromatography A to afford the title compound (500 mg, 95%) as colorless syrup.

Step 2: Synthesis of methyl 2-(bromomethyl)-5-cyclopropoxy-3-(trifluoromethyl)benzoate. A mixture of methyl 5-cyclopropoxy-2-methyl-3-(trifluoromethyl)benzoate (750 mg, 2.7 mmol), NBS (727.1 mg, 4.1 mmol) and BPO (199 mg, 0.78 mmol) in tetrachloromethane (20 mL) was stirred at 80° C. for 16 h. The solids were removed by filtration and the filtrate was concentrated under vacuum. The residue was purified by chromatography A to afford the title compound (176.8 mg, 18%) as a colorless oil.

Step 4: Synthesis of 6-cyclopropoxy-2-(3-{3-[(4-methyl-1,2,4-triazol-3-yl)methyl]oxetan-3-yl}phenyl)-4-(trifluoromethyl)-3H-isoindol-1-one. Methyl 2-(bromomethyl)-5-cyclopropoxy-3-(trifluoromethyl)benzoate (53 mg, 0.15 mmol) and 3-{3-[(4-methyl-1,2,4-triazol-3-yl)methyl]oxetan-3-yl}aniline (42 mg, 0.17 mmol) were coupled following the procedure from Example 260 step 2 to afford 44 mg (60%) of the title compound as an off-white solid: ¹H NMR (500 MHz, DMSO-d₆) δ 8.19 (s, 1H), 7.87 (ddd, J=8.2, 2.2, 0.9 Hz, 1H), 7.71 (d, J=2.2 Hz, 1H), 7.59 (d, J=2.2 Hz, 1H), 7.39 (t, J=2.0 Hz, 1H), 7.35 (t, J=8.0 Hz, 1H), 6.77 (dt, J=7.8, 1.2 Hz, 1H), 5.04 (d, J=1.7 Hz, 2H), 4.96 (d, J=6.1 Hz, 2H), 4.89 (d, J=6.0 Hz, 2H), 4.13 (tt, J=6.0, 2.9 Hz, 1H), 3.51 (s, 2H), 2.90 (s, 3H), 0.91-0.85 (m, 2H), 0.77-0.70 (m, 2H); LCMS: C₂₅H₂₃F₃N₄O₃ requires: 484, found: m/z=485 [M+H]⁺.

### Example 514: 6-(azetidin-3-yloxy)-2-(3-{3-[(4-methyl-1,2,4-triazol-3-yl)methyl]oxetan-3-yl}phenyl)-4-(trifluoromethyl)-3H-isoindol-1-one

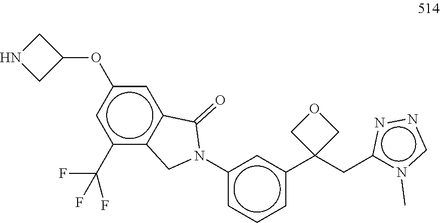

Step 1: Synthesis of methyl 2-methyl-5-(4,4,5,5-tetramethyl-1,3,2-dioxaborolan-2-yl)-3-(trifluoromethyl) benzoate. A degassed solution of methyl 5-bromo-2-methyl-3-(trifluoromethyl)benzoate (10.0 g, 33.7 mmol), B₂Pin₂ (9.4 g, 37.3 mmol), KOAc (9.91 g, 100 mmol) and Pd(dppf)Cl₂ (2.46 g, 3.36 mmol) in dioxane (100.0 mL) was stirred at 100° C. for 16 h under N₂ atmosphere. The mixture was cooled to rt and filtered. The filtrate was concentrated under vacuum. The residue was diluted with water followed by General Work-up Procedure 1. The residue was purified by chromatography A to afford the title compound (6.9 g, 59%) as an off-white solid.

Step 2: Synthesis of methyl 5-hydroxy-2-methyl-3-(trifluoromethyl)benzoate. To a suspension of methyl 2-methyl-5-(4,4,5,5-tetramethyl-1,3,2-dioxaborolan-2-yl)-3-(trifluoromethyl)benzoate (3.50 g, 10.2 mmol) and Oxone (5.6 g, 33.2 mmol) in acetone (35 mL) and water (50 mL) was added a solution of NaHCO₃ (6.28 g, 74.8 mmol) in water (100 mL). The mixture was stirred at rt for 16 h. The mixture was diluted water followed by General Work-up Procedure 1. The residue was purified by chromatography A to afford the title compound (1.25 g, 51%) as an off-white solid.

Step 3: Synthesis of tert-butyl 3-(3-(methoxycarbonyl)-4-methyl-5-(trifluoromethyl)phenoxy)azetidine-1-carboxylate. A mixture of methyl 5-hydroxy-2-methyl-3-(trifluoromethyl)benzoate (1.0 g, 4.27 mmol), tert-butyl 3-((methylsulfonyl)oxy)azetidine-1-carboxylate (1.3 g, 5.12 mmol), DMF (10 mL) and Cs₂CO₃ (2.0 g, 8.54 mmol). The mixture was stirred at 80° C. for 16 h. The reaction was quenched by the addition of water followed by General Work-up Procedure 1. The residue was purified by chromatography A to afford the title compound (1.1 g, 67%) as a colorless oil.

Step 4: Synthesis of tert-butyl 3-(4-(bromomethyl)-3-(methoxycarbonyl)-5-(trifluoromethyl)phenoxy)azetidine-1-carboxylate. To a solution of tert-butyl 3-(3-(methoxycarbonyl)-4-methyl-5-(trifluoromethyl)phenoxy)azetidine-1-carboxylate (650 mg, 1.67 mmol) in CCl₄ (20 mL) were added NBS (443 mg, 2.51 mmol) and BPO (121 mg, 0.50 mmol). The mixture was refluxed at 80° C. for 16 h. The mixture was concentrated under vacuum. The residue was purified by chromatography A and further purified by HPLC to afford the title compound (150 mg, 19%) as a light yellow semi-solid.

Step 5: Synthesis of tert-butyl 3-{[2-(3-{3-[(4-methyl-1,2,4-triazol-3-yl)methyl]-oxetan-3-yl}phenyl)-3-oxo-7-(trifluoromethyl)-1H-isoindol-5-yl]oxy}azetidine-1-carboxylate. tert-butyl 3-[4-(bromomethyl)-3-(methoxycarbonyl)-5-(trifluoromethyl)phenoxy]azetidine-1-carboxylate (50 mg, 0.11 mmol) and 3-{3-[(4-methyl-1,2,4-triazol-3-yl)methyl]oxetan-3-yl}aniline (26 mg, 0.11 mmol) were coupled following the procedure from Example 260 step 2 to afford 44 mg (60%) of the title compound as an off-white solid.

Step 6: Synthesis of 6-(azetidin-3-yloxy)-2-(3-{3-[(4-methyl-1,2,4-triazol-3-yl)methyl]oxetan-3-yl}phenyl)-4-(trifluoromethyl)-3H-isoindol-1-one. A solution of tert-butyl 3-{[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]oxy}azetidine-1-carboxylate (15 mg, 0.03 mmol) and HFIPA (1.0 mL) was heated at 100° C. in a microwave reactor for 4 h. The mixture was concentrated onto Celite and purified by chromatography C to afford 4 mg (32%) of the title compound as an off-white solid: ¹H NMR (500 MHz, DMSO-d₆) δ 8.18 (s, 1H), 7.85 (dd, J=8.2, 2.1 Hz, 1H), 7.49 (d, J=2.2 Hz, 1H), 7.39 (t, J=1.9 Hz, 1H), 7.38-7.30 (m, 2H), 6.77 (d, J=8.0 Hz, 1H), 5.26 (p, J=5.9 Hz, 1H), 5.03 (s, 2H), 4.96 (d, J=6.0 Hz, 2H), 4.88 (d, J=6.0 Hz, 2H), 4.01-3.91 (m, 2H), 3.63 (dd, J=9.7, 5.3 Hz, 2H), 3.51 (s, 2H), 2.90 (s, 3H); LCMS: C₂₅H₂₄F₃N₅O₃ requires: 499, found: m/z=500 [M+H]⁺.

### Example 515 and 516: 6-[(1R)-1-{5-azaspiro[2.4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one and 6-[(1S)-1-{5-azaspiro[2.4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one

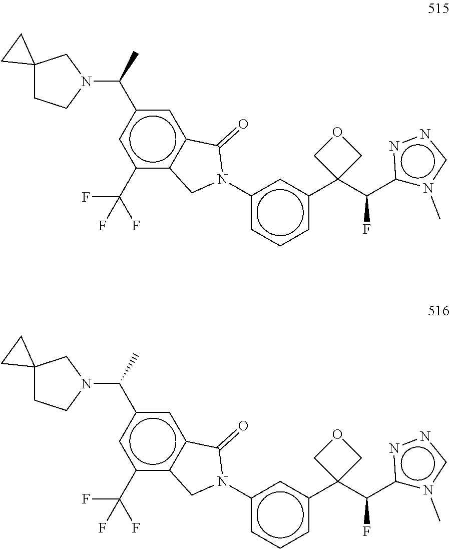

Step 1: Synthesis of 6-acetyl-2-(3-{3-[(S)-fluoro(4-methyl-1,2,4-triazol-3-yl)methyl]oxetan-3-yl}phenyl)-4-(trifluoromethyl)-3H-isoindol-1-one. The condensation between 3-{3-[(S)-fluoro(4-methyl-1,2,4-triazol-3-yl)methyl]oxetan-3-yl}aniline (75 mg, 0.29 mmol) and methyl 5-acetyl-2-(bromomethyl)-3-(trifluoromethyl)benzoate (97 mg, 0.29 mmol) was carried out according to the procedure from Example 260 step 2 to afford 115 mg (82%) of the title compound as a yellow solid.

Step 2: Synthesis of 6-[(1R)-1-{5-azaspiro[2.4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one and 6-[(1S)-1-{5-azaspiro[2.4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one. The reductive amination between 6-acetyl-2-(3-{3-[(S)-fluoro(4-methyl-1,2,4-triazol-3-yl)-methyl]oxetan-3-yl}phenyl)-4-(trifluoromethyl)-3H-isoindol-1-one (115 mg, 0.24 mmol) and 5-azaspiro[2.4]heptane hydrochloride (94 mg, 0.71 mmol) was carried out according to the procedure from Example 459 step 2 to afford (89 mg, 66%) of a mixture of diastereomers. The diastereomers (88 mg) were separated over an AS column eluting with 15% of (7:3MeOH:MeCN) 0.1% DEA in CO₂ to afford the first isomer (30 mg, 34%) as a colorless solid: ¹H NMR (500 MHz, DMSO-d₆) δ 8.35 (s, 1H), 8.01 (s, 1H), 7.97-7.90 (m, 2H), 7.54 (t, J=2.0 Hz, 1H), 7.38 (t, J=8.0 Hz, 1H), 6.97 (d, J=7.8 Hz, 1H), 6.28 (d, J=45.8 Hz, 1H), 5.37 (d, J=6.8 Hz, 1H), 5.21 (dd, J=6.3, 1.4 Hz, 1H), 5.16-5.03 (m, 3H), 4.83 (dd, J=6.3, 3.9 Hz, 1H), 3.54 (q, J=6.5 Hz, 1H), 3.29 (s, 2H), 3.18 (s, 3H), 2.74 (q, J=7.7 Hz, 1H), 2.32 (d, J=8.8 Hz, 1H), 1.74 (tt, J=12.1, 5.6 Hz, 2H), 1.34 (d, J=6.5 Hz, 3H), 0.54-0.42 (m, 4H); LCMS: C₃₀H₃₁F₄N₅O₂ requires: 569, found: m/z=570 [M+H]⁺. And Peak 2 (26 mg, 29%) as a colorless solid: ¹H NMR (500 MHz, DMSO-d₆) δ 8.36 (s, 1H), 8.02 (s, 1H), 7.99-7.90 (m, 2H), 7.56 (t, J=1.9 Hz, 1H), 7.39 (t, J=8.0 Hz, 1H), 6.98 (d, J=7.3 Hz, 1H), 6.29 (d, J=45.8 Hz, 1H), 5.38 (d, J=6.7 Hz, 1H), 5.26-5.20 (m, 1H), 5.17-5.03 (m, 3H), 4.84 (dd, J=6.2, 3.9 Hz, 1H), 3.56 (q, J=6.5 Hz, 1H), 3.19 (s, 3H), 2.75 (q, J=7.7 Hz, 1H), 2.33 (d, J=8.8 Hz, 1H), 1.81-1.67 (m, 2H), 1.35 (d, J=6.5 Hz, 3H), 0.59-0.41 (m, 4H); LCMS: C₃₀H₃₁F₄N₅O₂ requires: 569, found: m/z=570 [M+H]⁺.

### Example 517a and 517b: 6-{5-azaspiro[2.4]heptan-5-ylmethyl}-2-{3-[(3S)-3-[(4-methyl-1,2,4-triazol-3-yl)methyl]oxolan-3-yl]phenyl}-4-(trifluoromethyl)-3H-isoindol-1-one and 6-{5-azaspiro[2.4]heptan-5-ylmethyl}-2-{3-[(3R)-3-[(4-methyl-1,2,4-triazol-3-yl)methyl]oxolan-3-yl]phenyl}-4-(trifluoromethyl)-3H-isoindol-1-one

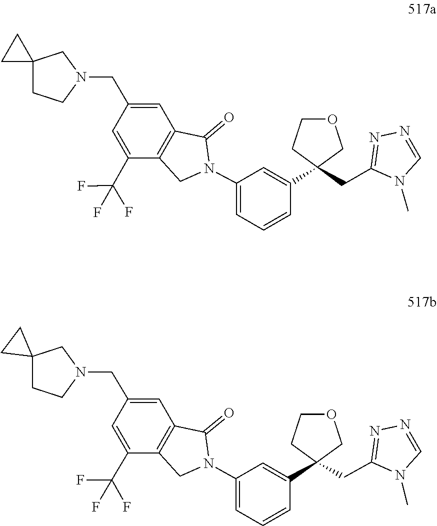 [SMILES: Cn1cnnc1C[C@@]1(c2cccc(N3Cc4c(cc(CN5CCC6(CC6)C5)cc4C(F)(F)F)C3=O)c2)CCOC1 ; Cn1cnnc1C[C@]1(c2cccc(N3Cc4c(cc(CN5CCC6(CC6)C5)cc4C(F)(F)F)C3=O)c2)CCOC1]

Step 1: Synthesis of ethyl 2-[3-(3-aminophenyl)oxolan-3-yl]-2-cyanoacetate. To a suspension of CuI (10.5 g, 55.2 mmol) in Et₂O (150 mL) was added 3-(chloromagnesio)-N,N-bis(trimethylsilyl)aniline (61.0 mL, 61.0 mmol, 2 M in THF) slowly at 0˜5° C. under nitrogen atmosphere. After stirring for 20 min, a solution of ethyl (E)-2-cyano-2-(dihydrofuran-3(2H)-ylidene)acetate (10.0 g, 55.2 mmol) in Et₂O (50 mL) was added dropwise to the above mixture at 0˜5° C. The mixture was warmed to rt and stirred for 16 h. The reaction was quenched by the addition of saturated NH₄Cl aqueous solution followed by General Work-up Procedure 1. The residue was purified by chromatography A to afford the title compound (6.2 g, 41%) as a brown oil.

Step 2: Synthesis of 2-[3-(3-[[(benzyloxy)carbonyl]amino]phenyl)oxolan-3-yl]acetic acid: A mixture of ethyl 2-[3-(3-aminophenyl)oxolan-3-yl]-2-cyanoacetate (8.5 g, 31.0 mmol) and KOH (19.0 g, 338.6 mmol) in ethane-1,2-diol (100 mL) was stirred at 150° C. for 7 h. The mixture was acidified by concentrated HCl to pH 6, and then basified by NaHCO₃ (sat) to pH 89. Then dioxane (100.0 mL) was added to the above mixture. The mixture was cooled to 0˜10° C. This was followed by the addition of Cbz-Cl (6.2 g) slowly at 0˜10° C. The mixture was stirred at rt for 2 h. The mixture was acidified by HCl (4 N) to pH 6 followed by General Work-up Procedure 1. The residue was purified by chromatography C to afford the title compound (5.5 g, 50%) as a brown oil.

Step 3: Synthesis of benzyl N-[3-[3-([[(methylcarbamothioyl)amino]carbamoyl]-methyl)oxolan-3-yl]phenyl]carbamate. To a mixture of 2-[3-(3-[[(benzyloxy)carbonyl]amino]-phenyl)oxolan-3-yl]acetic acid (4.5 g, 12.6 mmol) and 1-amino-3-methylthiourea (1.6 g, 15.2 mmol) were added EDCI (3.67 g, 19.1 mmol), HOAt (2.61 g, 19.2 mmol) and TEA (3.9 g, 38.5 mmol) sequentially at 0° C. The mixture was stirred at rt for 4 h. The mixture was diluted by water followed by General Work-up Procedure 1 to afford the title compound (5.3 g) as a brown syrup, which was used in the next step without purification.

Step 4: Synthesis of benzyl N-(3-[3-[(4-methyl-5-sulfanyl-4H-1,2,4-triazol-3-yl)methyl]oxolan-3-yl]phenyl)carbamate. To a solution of benzyl N-[3-[3-([[(methylcarbamothioyl)amino]carbamoyl]methyl)oxolan-3-yl]phenyl]carbamate (5.3 g, 12.0 mmol) in THF (100 mL) was added NaOH (aq, IN) (120 mL) slowly at 0˜10° C. The mixture was stirred at rt for 16 h. The mixture was acidified by HCl (4 N) to pH 6, followed by General Work-up Procedure 1 to afford the title compound (4.0 g) as a yellow syrup, which was used in the next step without purification.

Step 5: Synthesis of benzyl N-(3-[3-[(4-methyl-4H-1,2,4-triazol-3-yl)methyl]oxolan-3-yl]phenyl)carbamate. To a solution of benzyl N-(3-[3-[(4-methyl-5-sulfanyl-4H-1,2,4-triazol-3-yl)methyl]oxolan-3-yl]phenyl)carbamate (4.0 g, 9.4 mmol) in DCM (100 mL) was added AcOH (10 mL), then H₂O₂ (aq., 30%) (3.2 g, 94.1 mmol) was added slowly at 0˜10° C. The mixture was stirred at rt for 4 h. The reaction was quenched by Na₂S₂O₄ aqueous solution at 0 20° C. The mixture was then basified by saturated NaHCO₃ aqueous solution followed by General Work-up Procedure 1. The residue was purified by flash column chromatography with 0-20% MeOH in EtOAc to afford the title compound (1.2 g, 32%) as a white solid

Step 6: Synthesis of 3-(3-((4-methyl-4H-1,2,4-triazol-3-yl)methyl)THF-3-yl)aniline. A mixture of benzyl N-(3-[3-[(4-methyl-4H-1,2,4-triazol-3-yl)methyl]oxolan-3-yl]phenyl)carbamate (1.1 g, 2.8 mmol), MeOH (20 mL) and Pd/C (300 mg, 2.8 mmol, 10%) was stirred at rt for 2 h under hydrogen atmosphere. The solids were removed by filtration and the filtrate was concentrated under vacuum. The residue was purified by chromatography C to afford the title compound (655 mg, 90%) as a brown solid.

Step 7: Synthesis of 2-(3-{3-[(4-methyl-1,2,4-triazol-3-yl)methyl]oxolan-3-yl}phenyl)-3-oxo-7-(trifluoromethyl)-1H-isoindole-5-carbaldehyde. The condensation between 3-{3-[(4-methyl-1,2,4-triazol-3-yl)methy 1]oxolan-3-yl}aniline (250 mg, 0.97 mmol) and methyl 2-(bromomethyl)-5-formyl-3-(trifluoromethyl)benzoate (315 mg, 0.97 mmol) was carried out according to the procedure from Example 260 step 2 to afford 152 mg (33%) of the title compound as a yellow solid.

Step 8: Synthesis of 6-{5-azaspiro[2.4]heptan-5-ylmethyl}-2-(3-{3-[(4-methyl-1,2,4-triazol-3-yl)methyl]oxolan-3-yl}phenyl)-4-(trifluoromethyl)-3H-isoindol-1-one. The reductive amination between 2-(3-{3-[(4-methyl-1,2,4-triazol-3-yl)methyl]oxolan-3-yl}phenyl)-3-oxo-7-(trifluoromethyl)-1H-isoindole-5-carbaldehyde (150 mg, 0.32 mmol) and 5-azaspiro[2.4]heptane hydrochloride (130 mg, 0.96 mmol) was carried out in a similar fashion as for 447, step 4 to afford 108 mg of racemic material as a colorless solid. The enantiomers were resolved by SFC over an AZ column, eluting with 50% MeOH with 0.1% DEA in CO₂ to afford 41 mg (38%) of Peak 1 and 43 mg (41%) of Peak 2 as colorless solids. Peak 1: ¹H NMR (500 MHz, DMSO-d₆)δ 8.15 (s, 1H), 7.99 (s, 1H), 7.94 (s, 1H), 7.81 (dd, J=8.2, 2.0 Hz, 1H), 7.50 (d, J=2.0 Hz, 1H), 7.31 (t, J=8.0 Hz, 1H), 6.79 (d, J=7.7 Hz, 1H), 5.10 (s, 2H), 4.38 (d, J=8.3 Hz, 1H), 4.03 (q, J=7.9 Hz, 1H), 3.90 (td, J=8.8, 4.2 Hz, 1H), 3.83-3.77 (m, 3H), 3.10 (s, 2H), 2.82 (s, 3H), 2.70 (t, J=6.8 Hz, 2H), 2.68-2.60 (m, 2H), 2.19 (dt, J=12.3, 8.7 Hz, 1H), 1.77 (t, J=6.8 Hz, 2H), 0.50 (d, J=4.8 Hz, 4H); LCMS: C₃₀H₃₂F₃N₅O₂ requires: 552, found: m/z=553 [M+H]⁺. Peak 2: ¹H NMR (500 MHz, DMSO-d₆) δ 8.15 (s, 1H), 7.99 (s, 1H), 7.95 (s, 1H), 7.81 (dd, J=8.0, 2.1 Hz, 1H), 7.49 (t, J=1.9 Hz, 1H), 7.31 (t, J=8.0 Hz, 1H), 6.79 (d, J=7.8 Hz, 1H), 5.10 (s, 2H), 4.38 (d, J=8.3 Hz, 1H), 4.03 (q, J=7.9 Hz, 1H), 3.90 (td, J=8.7, 4.2 Hz, 1H), 3.85-3.75 (m, 3H), 3.10 (s, 2H), 2.82 (s, 3H), 2.70 (s, 2H), 2.68-2.59 (m, 2H), 2.19 (dt, J=12.4, 8.7 Hz, 1H), 1.77 (s, 2H), 0.58-0.44 (m, 4H); LCMS: C₃₀H₃₂F₃N₅O₂ requires: 552, found: m/z=553 [M+H]⁺.

### Example 518: 2-cyclopropyl-6-methyl-N-(3-(2-(4-methyl-4H-1,2,4-triazol-3-yl)cyclopent-1-en-1-yl)phenyl)pyrimidine-4-carboxamide

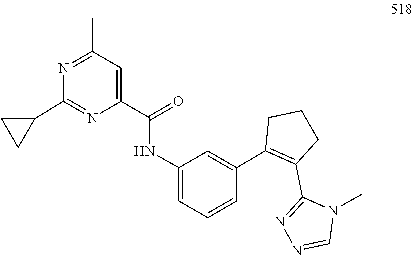 [SMILES: Cc1cc(C(=O)Nc2cccc(C3=C(c4nncn4C)CCC3)c2)nc(C2CC2)n1]

Step 1: Synthesis of methyl 2-(3-nitrophenyl)cyclopent-1-ene-1-carboxylate. A 100-mL flask was charged with methyl 2-(trifluoromethanesulfonyloxy)cyclopent-1-ene-1-carboxylate (0.946 mg, 3.45 mmol) (Logan, A. W., et al, Org. Lett., 2012, 14(12), 2940), 3-nitrophenylboronic acid (501 mg, 3.00 mmol), lithium chloride (509 mg, 12.0 mmol), tetrakis(triphenylphosphine)palladium (208 mg, 0.18 mmol) before the addition of 1,2-dimethoxyethane (25 mL) and aqueous sodium carbonate (3 mL of 1M solution). This reaction was then heated at 100° C. for 2 days before being cooled to room temperature. The mixture was diluted with water (50 mL) and worked up following General Work-up Procedure 1. The crude residue was purified by Chromatography A to provide the title compound as a colorless solid (325 mg, 44% yield).

Step 2: Synthesis of 2-(3-nitrophenyl)cyclopent-1-ene-1-carbohydrazide. The hydrazide formation reaction was carried out in a manner similar to Example D, Step 2, using methyl 2-(3-nitrophenyl)cyclopent-1-ene-1-carboxylate (325 mg, 1.31 mmol) and performing the reaction at 80° C. to provide the title compound (300 mg, 92% yield).

Step 3: Synthesis of 4-methyl-5-[2-(3-nitrophenyl)cyclopent-1-en-1-yl]-1,2,4-triazole-3-thiol. The following reaction was carried out in a manner similar to Example S using 2-(3-nitrophenyl)cyclopent-1-ene-1-carbohydrazide (300 mg, 1.2 mmol) and heating at 70° C. for 16 h, before addition of sodium hydroxide. After following General Work-up Procedure 1, the crude residue was purified Chromatography B to afford the title compound (125 mg, 31% yield).

Step 4: Synthesis of 4-methyl-3-[2-(3-nitrophenyl)cyclopent-1-en-1-yl]-1,2,4-triazole. The triazole formation was carried out in a manner similar to Example S using 4-methyl-5-[2-(3-nitrophenyl)cyclopent-1-en-1-yl]-1,2,4-triazole-3-thiol (125 mg, 0.41 mmol). The reaction was then diluted with dichloromethane and poured into saturated sodium bicarbonate. The aqueous layer was subsequently extracted with a dichloromethane:isopropanol (9:1) mixture (3×15 mL) and combined organics were dried over sodium sulfate and concentrated under reduced pressure. The crude residue was purified by Chromatography B to yield the title compound (51 mg, 45% yield).

Step 5: Synthesis of 3-[2-(4-methyl-1,2,4-triazol-3-yl)cyclopent-1-en-1-yl]aniline. The nitro reduction was carried out in a manner similar to Example 470, Step 3, using 4-methyl-3-[2-(3-nitrophenyl)cyclopent-1-en-1-yl]-1,2,4-triazole (100 mg, 0.37 mmol) to provide the title compound (45 mg, 51% yield).

Step 6: Synthesis of 2-cyclopropyl-6-methyl-N-(3-(2-(4-methyl-4H-1,2,4-triazol-3-yl)cyclopent-1-en-1-yl)phenyl)pyrimidine-4-carboxamide. The amide bond formation reaction was carried out in a similar fashion as for Example 74 using 3-[2-(4-methyl-1,2,4-triazol-3-yl)cyclopent-1-en-1-yl]aniline (21 mg, 0.08 mmol) and 2-cyclopropyl-6-methylpyrimidine-4-carboxylic acid (15 mg, 0.08 mmol) in acetonitrile (0.67 mL) and dimethylformamide (0.16 mL) as solvents. The title compound was obtained as a colorless film (4.5 mg, 13% yield) after using Chromatography C. LCMS: C₂₃H₂₄N₆O requires 400.2, found 401.5 [M+H]⁺. ¹H NMR (500 MHz, DMSO-d6) δ 10.45 (s, 1H), 8.87 (s, 1H), 7.83-7.70 (m, 1H), 7.67 (d, J=2.9 Hz, 2H), 7.32 (t, J=7.9 Hz, 1H), 6.84 (dt, J=7.9, 1.3 Hz, 1H), 3.24 (s, 3H), 3.09-2.97 (m, 2H), 2.96-2.83 (m, 2H), 2.52 (s, 3H), 2.31 (tt, J=8.0, 4.7 Hz, 1H), 2.13 (p, J=7.7 Hz, 2H), 1.22-1.04 (m, 4H).

### Example 519: 4-((5-azaspiro[2.4]heptan-5-yl)methyl)-6-cyclopropyl-N-(3-(3-((4-methyl-4H-1,2,4-triazol-3-yl)methyl)oxetan-3-yl)phenyl)picolinamide

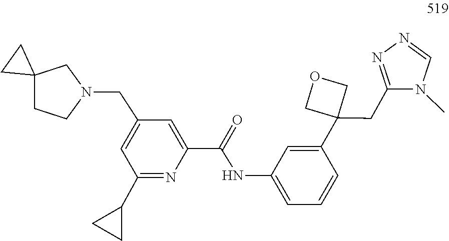 [SMILES: Cn1cnnc1CC1(c2cccc(NC(=O)c3cc(CN4CCC5(CC5)C4)cc(C4CC4)n3)c2)COC1]

Step 1: Synthesis of 4-((5-azaspiro[2.4]heptan-5-yl)methyl)-6-cyclopropyl-N-(3-(3-((4-methyl-4H-1,2,4-triazol-3-yl)methyl)oxetan-3-yl)phenyl)picolinamide. The reaction was carried out in a similar fashion as for Example 74 using 4-((5-azaspiro[2.4]heptan-5-yl)methyl)-6-cyclopropylpicolinic acid (500, step 2) (210 mg, 0.77 mmol) and 3-(3-[(4-methyl-1,2,4-triazol-3-yl)methyl]oxetan-3-yl)aniline (Example R) (188 mg, 0.77 mol) in DMF (1.5 mL) and acetonitrile (6.1 mL) as solvents. Reaction was heated at 50° C. for one hour before stirring at RT overnight. Reaction was then diluted with acetonitrile and water, concentrated onto Celite and purified using Chromatography C. The title compound was isolated as a white solid (187 mg, 49% yield). LCMS: C₂₉H₃₄N₆O₂ requires 498.3, found 499.6 [M+H]⁺. ¹H NMR (500 MHz, DMSO-d6) δ 10.11 (s, 1H), 8.20 (s, 1H), 7.83 (d, J=1.3 Hz, 1H), 7.73 (ddd, J=8.1, 2.1, 1.0 Hz, 1H), 7.50-7.37 (m, 2H), 7.27 (t, J=7.9 Hz, 1H), 6.69-6.60 (m, 1H), 4.94 (d, J=5.9 Hz, 2H), 4.86 (d, J=6.0 Hz, 2H), 3.67 (s, 2H), 3.49 (s, 2H), 2.92 (s, 3H), 2.68 (t, J=6.8 Hz, 2H), 2.44 (s, 2H), 2.24 (tt, J=8.2, 4.8 Hz, 1H), 1.77 (t, J=6.8 Hz, 2H), 1.21-1.09 (m, 2H), 1.09-0.97 (m, 2H), 0.51 (dt, J=8.4, 2.0 Hz, 4H). 2-cyclopropyl-6-methyl-N-(3-(3-methyl-5-(4-methyl-4H-1,2,4-triazol-3-yl)-1H-pyrazol-1-yl)phenyl)pyrimidine-4-carboxamide

### Example 520: 6-((5-azaspiro[2.4]heptan-5-yl)methyl)-2-(3-(3-((4-methyl-4H-1,2,4-triazol-3-yl)methyl)oxetan-3-yl)phenyl)-4-(trifluoromethyl)isoindolin-1-one

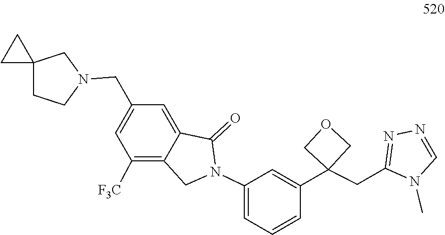 [SMILES: Cn1cnnc1CC1(c2cccc(N3Cc4c(cc(CN5CCC6(CC6)C5)cc4C(F)(F)F)C3=O)c2)COC1]

Step 1: Synthesis of 2-(3-{3-[(4-methyl-1,2,4-triazol-3-yl)methyl]oxetan-3-yl}phenyl)-3-oxo-7-(trifluoromethyl)-1H-isoindole-5-carbaldehyde, The indolone formation reaction was carried out analogously to 260, Step 2 by combining 3-(3-((4-methyl-4H-1,2,4-triazol-3-yl)methyl)oxetan-3-yl)aniline (Example R) (11.3 g, 46.1 mmol) with methyl 2-(bromomethyl)-5-formyl-3-(trifluoromethyl)benzoate (Example V) (15.0 g, 46.1 mmol) in acetonitrile (410 mL) and water (205 mL). This was cooled to 0° C. before the addition of silver nitrate (10.2 g, 60.0 mmol) dissolved in 58 mL water. The reaction was stirred for 40 h at room temperature at which point solid sodium bicarbonate was added until solution was pH8. The mixture was then filtered through Celite, rinsing with acetonitrile (300 mL) followed by a dichloromethane:ethyl acetate mixture (300 mL, 9:1). The organic layer was separated and dried over sodium sulfate. The crude residue was purified by Chromatography B. This oil obtained was then azeotroped with toluene (3×150 mL) to afford the title compound (10.5 g, 50%) as a light-yellow solid.

Step 2: Synthesis of 6-((5-azaspiro[2.4]heptan-5-yl)methyl)-2-(3-(3-((4-methyl-4H-1,2,4-triazol-3-yl)methyl)oxetan-3-yl)phenyl)-4-(trifluoromethyl)isoindolin-1-one. To a solution of 2-(3-{3-[(4-methyl-1,2,4-triazol-3-yl)methyl]oxetan-3-yl}phenyl)-3-oxo-7-(trifluoromethyl)-1H-isoindole-5-carbaldehyde (7.50 g, 16.4 mmol) in dichloroethane (60 mL) was added 5-azaspiro[2.4]heptane hydrochloride (4.39 g, 32.9 mmol) followed by triethylamine (3.21 mL, 23.0 mmol). This mixture was stirred for 15 min at room temperature before the addition of sodium triacetoxyborohydride (5.22 g, 24.7 mmol, added in 3 equal portions over 2 hours). The reaction was stirred vigorously overnight before being quenched with saturated aqueous sodium bicarbonate. This was extracted with dichloromethane (3×200 mL) and the combined organics were washed with brine and dried over sodium sulfate. The crude residue was purified by silica gel column chromatography using Chromatography B (containing 0.1% conc, ammonium hydroxide). Fractions containing the desired material were concentrated onto Celite and further purified Chromatography C to afford pure title compound as a white solid (5.29 g, 60% yield). LCMS: (C₂₉H₃₀F₃N₅O₂) requires 537.2, found: 538.3 [M+H]⁺. ¹H NMR (500 MHz, DMSO-d6) δ 8.20 (s, 1H), 8.00 (s, 1H), 7.95 (s, 1H), 7.89 (dd, J=8.2, 2.2 Hz, 1H), 7.41 (t, J=2.0 Hz, 1H), 7.36 (t, J=7.9 Hz, 1H), 6.77 (d, J=7.7 Hz, 1H), 5.11 (s, 2H), 4.98 (d, J=6.0 Hz, 2H), 4.90 (d, J=6.0 Hz, 2H), 3.81 (s, 2H), 3.52 (s, 2H), 2.91 (s, 3H), 2.71 (t, J=6.8 Hz, 2H), 2.46 (s, 2H), 1.78 (t, J=6.8 Hz, 2H), 0.59-0.44 (m, 4H).

### Example 521: 6-{[2-(2-fluoroethyl)morpholin-4-yl]methyl}-2-(3-{3-[(4-methyl-1,2,4-triazol-3-yl)methyl]oxetan-3-yl}phenyl)-4-(trifluoromethyl)-3H-isoindol-1-one

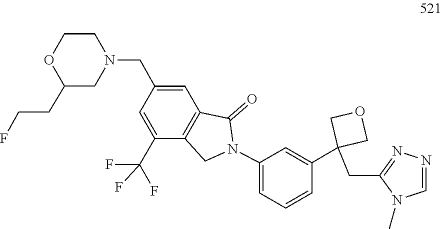 [SMILES: Cn1cnnc1CC1(c2cccc(N3Cc4c(cc(CN5CCOC(CCF)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)-isoindoline-5-carbaldehyde (100 mg, 0.22 mmol) and 2-(2-fluoroethyl)morpholine (88 mg, 0.66 mmol) as reactants to afford the title compound (84 mg, 64%) as a white solid. ¹H NMR (500 MHz, Acetonitrile-d₃) δ 8.21 (s, 1H), 8.16 (d, J=7.5 Hz, 2H), 7.92 (dd, J=8.1, 2.2 Hz, 1H), 7.42 (dd, J=7.9 Hz, 1H), 7.40-7.33 (m, 1H), 6.85 (d, J=7.8, 1.4 Hz, 1H), 5.06 (s, 2H), 5.04-5.00 (m, 4H), 4.65-4.56 (m, 1H), 4.56-4.47 (m, J=4.2 Hz, 1H), 4.45-4.36 (m, 2H), 4.03 (dd, J=13.1, 3.8 Hz, 1H), 3.96 (s, 1H), 3.93-3.85 (m, 1H), 3.66 (s, 2H), 3.34 (t, J=12.0 Hz, 2H), 3.04 (d, J=11.6 Hz, 1H), 2.96 (s, 3H), 2.80 (t, J=11.6 Hz, 1H), 1.95-1.77 (m, 2H); LCMS: C₂₉H₃₁F₄N₅O₃ requires: 573, found: m/z=574 [M+H]⁺.

### Example 522: 2-cyclopropyl-6-methyl-N-(3-(l-methyl-4-(4-methyl-4H-1,2,4-triazol-3-yl)-1H-pyrazol-5-yl)phenyl)pyrimidine-4-carboxamide

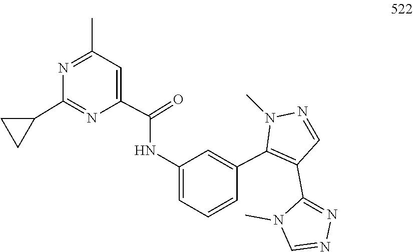 [SMILES: Cc1cc(C(=O)Nc2cccc(-c3c(-c4nncn4C)cnn3C)c2)nc(C2CC2)n1]

Step 1: Synthesis of ethyl 1-methyl-5-(3-nitrophenyl)pyrazole-4-carboxylate. A solution of ethyl 5-bromo-1-methylpyrazole-4-carboxylate (1.0 g, 4.29 mmol), 3-nitrophenylboronic acid (859 mg, 5.15 mmol), sodium carbonate (909 mg, 8.58 mmol dissolved in 4.3 mL water), and bis(triphenylphosphine)palladium(II) dichloride (151 mg, 0.21 mmol) in DME (43 mL) was heated to 90° C. overnight. After this time, General Work-up Procedure 1 was followed, and the crude residue was purified by Chromatography A to afford the title compound (710 mg, 60% yield).

Step 2: Synthesis of l-methyl-5-(3-nitrophenyl)pyrazole-4-carboxylic acid. Lithium hydroxide hydrate (119 mg, 2.84 mmol) was added to a solution of ethyl 1-methyl-5-(3-nitrophenyl)pyrazole-4-carboxylate (710 mg, 2.58 mmol) in THF (10.3 mL), water (10.3 mL), and methanol (2.0 mL). This solution was heated to 50° C. for 5 hr. before being concentrated to dryness. This residue was dissolved in EtOAc and washed with 0.1M HCl and the aqueous phase extracted 3× with EtOAc. The combined organics were dried, and concentrated to a yellow solid (600 mg, 2.43 mmol) which was used without purification.

Step 3: Synthesis of 4-methyl-5-[1-methyl-5-(3-nitrophenyl)pyrazol-4-yl]-1,2,4-triazole-3-thiol. To a solution of l-methyl-5-(3-nitrophenyl)pyrazole-4-carboxylic acid (600 mg, 2.43 mmol), 4-methyl-3-thiosemicarbazide (319 mg, 3.03 mmol), and N,N-diisopropylethylamine (0.42 mL, 2.43 mmol) in DMF (6.4 mL) was added HATU (1.10 g, 2.91 mmol). The reaction was stirred for 4 hr. before the addition of 1M sodium hydroxide (5.1 mL). This solution was stirred at 50° C. overnight. After cooling to RT, saturated ammonium chloride (30 mL) was added and the mixture stirred 15 min. The solids were filtered, rinsed with water, and dried under vacuum to provide the title compound (600 mg, 1.90 mmol).

Step 4: Synthesis of 3-[2-methyl-4-(4-methyl-1,2,4-triazol-3-yl)pyrazol-3-yl]aniline. The reduction was carried out similar to Example 521, Step 3, using 4-methyl-5-[1-methyl-5-(3-nitrophenyl)pyrazol-4-yl]-1,2,4-triazole-3-thiol (200 mg, 0.63 mmol) to afford the title compound as a yellow solid.

Step 5: Synthesis of 2-cyclopropyl-6-methyl-N-(3-(l-methyl-4-(4-methyl-4H-1,2,4-triazol-3-yl)-1H-pyrazol-5-yl)phenyl)pyrimidine-4-carboxamide. The amide formation was carried out like in Example 168, Step 1, using 3-[2-methyl-4-(4-methyl-1,2,4-triazol-3-yl)pyrazol-3-yl]aniline (46 mg, 0.18 mmol) and 2-cyclopropyl-6-methylpyrimidine-4-carboxylic acid (32 mg, 0.18 mmol). The title compound was obtained after Chromatography B as a white solid (37 mg, 0.09 mmol). LCMS: C₂₂H₂₂N₈O requires 414.2, found 415.3 [M+H]⁺. ¹H NMR (500 MHz, Acetonitrile-d₃) δ 10.02 (s, 1H), 8.11 (s, 1H), 7.95-7.86 (m, 1H), 7.86-7.79 (m, 1H), 7.73 (d, J=1.1 Hz, 2H), 7.46 (t, J=7.9 Hz, 1H), 7.16 (dt, J=7.5, 1.3 Hz, 1H), 3.87 (s, 3H), 3.33 (s, 3H), 2.52 (s, 3H), 2.30 (tt, J=8.1, 4.7 Hz, 1H), 1.23-1.15 (m, 2H), 1.15-1.01 (m, 2H).

### Example 523: 2-(3-(l-methyl-4-(4-methyl-4H-1,2,4-triazol-3-yl)-1H-pyrazol-5-yl)phenyl)-4-(trifluoromethyl)isoindolin-1-one

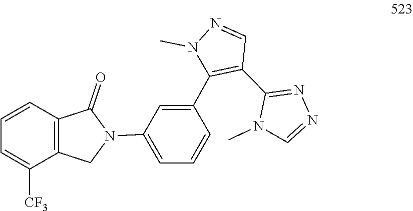 [SMILES: Cn1cnnc1-c1cnn(C)c1-c1cccc(N2Cc3c(cccc3C(F)(F)F)C2=O)c1]

Synthesis of 2-(3-(l-methyl-4-(4-methyl-4H-1,2,4-triazol-3-yl)-1H-pyrazol-5-yl)phenyl)-4-(trifluoromethyl)isoindolin-1-one. The cyclization was carried out similar to 521, Step 1, using 3-[2-methyl-4-(4-methyl-1,2,4-triazol-3-yl)pyrazol-3-yl]aniline (51 mg, 0.20 mmol) and methyl 2-(bromomethyl)-3-(trifluoromethyl)benzoate (60 mg, 0.20 mmol)). The title compound was obtained after Chromatography B as a white solid (17 mg, 0.04 mmol). LCMS: C₂₂H₁₇F₃N₆O requires 438.1, found 439.3 [M+H]⁺. ¹H NMR (500 MHz, Acetonitrile-d₃) δ 8.12 (s, 1H), 8.05 (d, J=7.6 Hz, 1H), 8.01 (ddd, J=8.3, 2.3, 1.0 Hz, 1H), 7.97 (t, J=2.0 Hz, 1H), 7.96-7.92 (m, 1H), 7.77-7.70 (m, 2H), 7.52 (t, J=8.0 Hz, 1H), 7.21 (dt, J=7.7, 1.1 Hz, 1H), 5.06 (s, 2H), 3.90 (s, 3H), 3.36 (s, 3H).

### Example 524: (R)-2-(3-(l-methyl-4-(4-methyl-4H-1,2,4-triazol-3-yl)-1H-pyrazol-5-yl)phenyl)-6-((2-methylmorpholino)methyl)-4-(trifluoromethyl)isoindolin-1-one

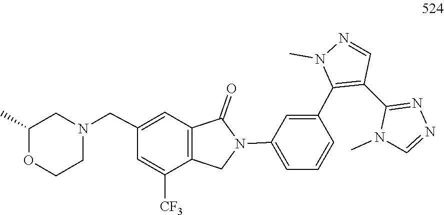 [SMILES: C[C@@H]1CN(Cc2cc3c(c(C(F)(F)F)c2)CN(c2cccc(-c4c(-c5nncn5C)cnn4C)c2)C3=O)CCO1]

Synthesis of (R)-2-(3-(1-methyl-4-(4-methyl-4H-1,2,4-triazol-3-yl)-1H-pyrazol-5-yl)phenyl)-6-((2-methylmorpholino)methyl)-4-(trifluoromethyl)isoindolin-1-one. The reductive amination was carried out like in Example 447, Step 4, using 2-(3-(1-methyl-4-(4-methyl-4H-1,2,4-triazol-3-yl)-1H-pyrazol-5-yl)phenyl)-3-oxo-7-(trifluoromethyl)isoindoline-5-carbaldehyde (35 mg, 0.08 mmol) and (R)-2-methylmorpholine hydrochloride (16 mg, 0.11 mmol). The title compound was obtained after Chromatography C as a white solid (17 mg, 0.03 mmol). LCMS: C₂₈H₂₈F₃N₇O₂ requires 551.2, found 552.4 [M+H]⁺. ¹H NMR (500 MHz, Acetonitrile-d3) δ 8.11 (s, 1H), 7.98 (tt, J=11.1, 2.2 Hz, 3H), 7.90 (s, 1H), 7.74 (s, 1H), 7.51 (t, J=8.0 Hz, 1H), 7.20 (dt, J=7.7, 1.3 Hz, 1H), 5.03 (d, J=1.6 Hz, 2H), 3.90 (s, 3H), 3.77 (ddd, J=11.5, 3.3, 1.6 Hz, 1H), 3.70-3.52 (m, 4H), 3.36 (s, 3H), 2.70 (dt, J=11.1, 2.1 Hz, 1H), 2.62 (dt, J=11.3, 2.0 Hz, 1H), 2.19-2.09 (m, 4H), 1.85-1.79 (m, 2H), 1.05 (d, J=6.2 Hz, 3H).

### Example 525: 6-(((2S,6S)-2,6-dimethylmorpholino)methyl)-2-(3-(l-methyl-4-(4-methyl-4H-1,2,4-triazol-3-yl)-1H-pyrazol-5-yl)phenyl)-4-(trifluoromethyl)isoindolin-1-one

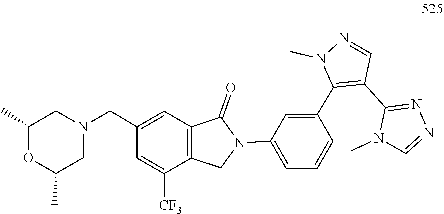 [SMILES: C[C@@H]1CN(Cc2cc3c(c(C(F)(F)F)c2)CN(c2cccc(-c4c(-c5nncn5C)cnn4C)c2)C3=O)C[C@H](C)O1]

Synthesis of 6-(((2R,6S)-2,6-dimethylmorpholino)methyl)-2-(3-(l-methyl-4-(4-methyl-4H-1,2,4-triazol-3-yl)-1H-pyrazol-5-yl)phenyl)-4-(trifluoromethyl)isoindolin-1-one. The reductive amination was carried out like in Example 447, Step 4, using 2-(3-(1-methyl-4-(4-methyl-4H-1,2,4-triazol-3-yl)-1H-pyrazol-5-yl)phenyl)-3-oxo-7-(trifluoromethyl)isoindoline-5-carbaldehyde (40 mg, 0.09 mmol), (2R,6S)-2,6-dimethyl morpholine (20 mg, 0.17 mmol)), and acetic acid in place of triethylamine. The title compound was obtained after Chromatography C as a white solid (20 mg, 0.04 mmol). LCMS: C₂₉H₃₀F₃N₇O₂ requires 565.2, found 566.5 [M+H]⁺. ¹H NMR (500 MHz, Acetonitrile-d3) δ 8.11 (s, 1H), 8.05-7.93 (m, 3H), 7.89 (s, 1H), 7.74 (s, 1H), 7.52 (t, J=7.9 Hz, 1H), 7.21 (dt, J=7.7, 1.2 Hz, 1H), 5.04 (s, 2H), 3.90 (s, 3H), 3.68-3.58 (m, 4H), 3.36 (s, 3H), 2.69 (dt, J=10.5, 1.9 Hz, 2H), 1.76 (t, J=10.7 Hz, 2H), 1.06 (d, J=6.2 Hz, 6H).

### Example 526: 2-cyclopropyl-N-(3-(3,3-difluoro-1-((4-methyl-4H-1,2,4-triazol-3-yl)methyl)cyclobutyl)phenyl)-6-methylpyrimidine-4-carboxamide

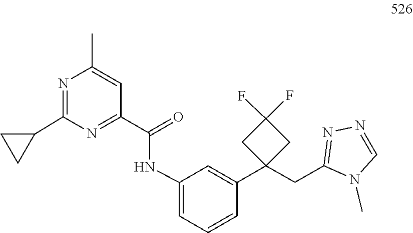 [SMILES: Cc1cc(C(=O)Nc2cccc(C3(Cc4nncn4C)CC(F)(F)C3)c2)nc(C2CC2)n1]

Step 1: Synthesis of ethyl 2-[3-(acetyloxy)-1-(3-bromophenyl)cyclobutyl]acetate. Aqueous KOH (22 mL, 1.5 M, 58.38 mmol) was added to a suspension of [Rh(COD)CI]₂ (850.0 mg, 1.73 mmol) in dioxane (15 ml) and the mixture was stirred for 30 min. before (3-bromophenyl)boronic acid (14.3 g, 71.04 mmol) and successively ethyl 2-[3-(acetyloxy)cyclobutylidene]acetate (5.7 g, 28.76 mmol) (Liang, Yin, et al., US Patent 20170290800 A1, Oct. 12, 2017) in dioxane (45 mL) were added and the reaction mixture was stirred at room temperature for 16 h. The reaction was quenched by the addition of HCl (1 A) to pH=67. After this time, General Work-up Procedure 1 was followed and the crude residue was purified by Chromatography A to afford the title compound (5.9 g, 57%) as a yellow oil.

Step 2: Synthesis of ethyl 2-[1-(3-bromophenyl)-3-hydroxycyclobutyl]acetate. A mixture of ethyl 2-[3-(acetyloxy)-1-(3-bromophenyl)cyclobutyl]acetate (4.0 g, 11.27 mmol) and K₂CO₃ (3.1 g, 22.54 mmol) in ethanol (50.0 mL) was stirred at room temperature for 3 h. The solvent was removed under vacuum. General Work-up Procedure 1 was followed and volatiles were concentrated under vacuum to afford the title compound (2.7 g, crude) as a colorless oil, which was used without purification.

Step 3: Synthesis of ethyl 2-[1-(3-bromophenyl)-3-oxocyclobutyl]acetate. A mixture of ethyl 2-[1-(3-bromophenyl)-3-hydroxycyclobutyl]acetate (2.7 g, 8.65 mmol) and IBX (3.6 g, 12.98 mmol) in EtOAc (30 mL) was stirred at 80° C. for 24 h. The solids were filtered and the filtrate was concentrated under vacuum. The crude residue was purified by Chromatography A to afford the title compound (1.7 g, 48% over two steps) as a yellow solid.

Step 4: Synthesis of ethyl 2-[1-(3-bromophenyl)-3,3-difluorocyclobutyl]acetate. To a solution of ethyl 2-[1-(3-bromophenyl)-3-oxocyclobutyl]acetate (1.7 g, 5.48 mmol) in dichloromethane (40.0 mL) was added diethylaminosulfur trifluoride (13.2 g, 82.25 mmol) dropwise at room temperature. The reaction was stirred at room temperature for 16h. The reaction was quenched by the addition of aqueous saturated sodium bicarbonate and General Work-up Procedure 1 was followed using dichloromethane. crude residue was purified by Chromatography A to afford the title compound (860.0 mg, 47%) as a yellow syrup.

Step 5: Synthesis of 2-[1-(3-bromophenyl)-3,3-difluorocyclobutyl]acetohydrazide. The hydrazide formation reaction was carried out in a manner like Example D, Step 2, using ethyl 2-[1-(3-bromophenyl)-3,3-difluorocyclobutyl]acetate (860.0 mg, 2.59 mmol) performing the reaction at 80° C. to provide the title compound.

Step 6: 5-[[1-(3-bromophenyl)-3,3-difluorocyclobutyl]methyl]-4-methyl-4H-1,2,4-triazole-3-thiol. The following reaction was carried out in a manner similar to Example S using 2-[1-(3-bromophenyl)-3,3-difluorocyclobutyl]acetohydrazide (890.0 mg, crude) to afford the title compound as a white solid (290.0 mg, crude).

Step 7: Synthesis of 3-((1-(3-bromophenyl)-3,3-difluorocyclobutyl)methyl)-4-methyl-4H-1,2,4-triazole. To a mixture of 5-[[1-(3-bromophenyl)-3,3-difluorocyclobutyl]methyl]-4-methyl-4H-1,2,4-triazole-3-thiol (290.0 mg, crude) and NaNO₂ (534.6 mg, 7.75 mmol) was added HNO₃ (1 N, 8.00 mL). The resulting mixture was stirred at room temperature for 1 h. The reaction was quenched with saturated aqueous sodium bicarbonate solution and General Work-up Procedure 1 was followed and the crude residue was purified by Chromatography C to afford the title compound as a white solid (143 mg).

Step 8: Synthesis of 2-cyclopropyl-N-(3-(3,3-difluoro-1-((4-methyl-4H-1,2,4-triazol-3-yl)methyl)cyclobutyl)phenyl)-6-methylpyrimidine-4-carboxamide. The reaction was performed in a similar fashion to Example 168, Step 1, using 3-((1-(3-bromophenyl)-3,3-difluorocyclobutyl)methyl)-4-methyl-4H-1,2,4-triazole (35 mg, 0.10 mmol) and 2-cyclopropyl-6-methylpyrimidine-4-carboxamide (23 mg, 0.13 mmol) to provide the title compound after Chromatography B with 0.1% ammonium hydroxide additive. LCMS: C₂₃H₂₄N₆O requires 438.2, found 439.4 [M+H]⁺. ¹H NMR (500 MHz, Chloroform-d) δ 9.81 (s, 1H), 7.84 (s, 1H), 7.75 (s, 1H), 7.57 (dd, J=8.1, 1.2 Hz, 1H), 7.41 (t, J=2.0 Hz, 1H), 7.26 (d, J=15.8 Hz, 1H), 6.61 (dt, J=8.1, 1.1 Hz, 1H), 3.42-3.30 (m, 2H), 3.27 (s, 2H), 3.00 (td, J=15.4, 12.7 Hz, 2H), 2.70 (s, 3H), 2.57 (s, 3H), 2.33 (tt, J=7.9, 4.7 Hz, 1H), 1.24-1.17 (m, 2H), 1.15 (dtd, J=7.7, 5.5, 4.8, 2.8 Hz, 2H).

### Example 527: 2-(3-(3,3-difluoro-1-((4-methyl-4H-1,2,4-triazol-3-yl)methyl)cyclobutyl)-phenyl)-4-(trifluoromethyl)isoindolin-1-one

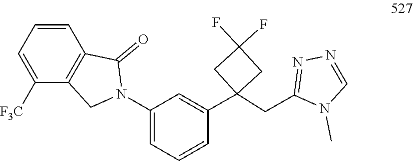 [SMILES: Cn1cnnc1CC1(c2cccc(N3Cc4c(cccc4C(F)(F)F)C3=O)c2)CC(F)(F)C1]

Synthesis of 2-(3-(3,3-difluoro-1-((4-methyl-4H-1,2,4-triazol-3-yl)methyl)cyclo-butyl)phenyl)-4-(trifluoromethyl)isoindolin-1-one. The reaction was performed in a similar fashion to Example 168, Step 1, using 3-((1-(3-bromophenyl)-3,3-difluorocyclobutyl)methyl)-4-methyl-4H-1,2,4-triazole (30 mg, 0.09 mmol) and 4-(trifluoromethyl)isoindolin-1-one (22 mg, 0.11 mmol) to provide the title compound after Chromatography B as a white solid (12 mg, 0.03 mmol). LCMS: C₂₃H₁₉F₅N₄O requires 462.1, found 463.5 [M+H]⁺. ¹H NMR (500 MHz, Chloroform-d) δ 8.10 (d, J=7.6 Hz, 1H), 7.94-7.80 (m, 2H), 7.68 (t, J=7.7 Hz, 1H), 7.63 (t, J=1.9 Hz, 1H), 7.51 (dd, J=8.2, 2.1 Hz, 1H), 7.31 (t, J=7.9 Hz, 1H), 6.68 (d, J=8.1 Hz, 1H), 4.95 (s, 2H), 3.37 (td, J=14.3, 6.3 Hz, 2H), 3.29 (s, 2H), 3.01 (td, J=15.3, 12.7 Hz, 2H), 2.75 (s, 3H).

### Example 528: 6-((4,4-difluoroazepan-1-yl)methyl)-2-(3-(3-((4-methyl-4H-1,2,4-triazol-3-yl)methyl)oxetan-3-yl)phenyl)-4-(trifluoromethyl)isoindolin-1-one

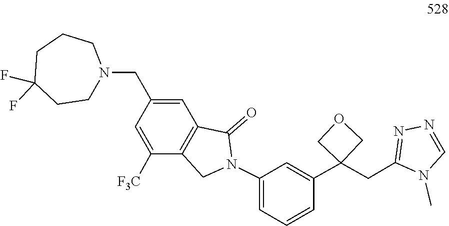 [SMILES: Cn1cnnc1CC1(c2cccc(N3Cc4c(cc(CN5CCCC(F)(F)CC5)cc4C(F)(F)F)C3=O)c2)COC1]

Synthesis of 6-((4,4-difluoroazepan-1-yl)methyl)-2-(3-(3-((4-methyl-4H-1,2,4-triazol-3-yl)methyl)oxetan-3-yl)phenyl)-4-(trifluoromethyl)isoindolin-1-one. The reductive animation was carried out in a similar fashion to Example 447, Step 4, using 2-(3-{3-[(4-methyl-1,2,4-triazol-3-yl)methyl]oxetan-3-yl}phenyl)-3-oxo-7-(trifluoromethyl)-1H-isoindole-5-carbaldehyde (50 mg, 0.11 mmol) and 4,4-difluoroazepane hydrochloride (47 mg, 0.27 mmol) to provide the title compound following Chromatography C. Isolated as a white solid (37 mg, 0.06 mmol). LCMS: C₂₉H₃₀F₅N₅O₂ requires 575.2, found 576.5 [M+H]⁺. ¹H NMR (500 MHz, DMSO-d₆) δ 8.18 (s, 1H), 8.01 (s, 1H), 7.96 (s, 1H), 7.93-7.85 (m, 1H), 7.40 (t, J=2.0 Hz, 1H), 7.35 (t, J=7.9 Hz, 1H), 6.76 (dt, J=7.9, 1.2 Hz, 1H), 5.10 (s, 2H), 4.96 (d, J=6.0 Hz, 2H), 4.89 (d, J=6.1 Hz, 2H), 3.84 (s, 2H), 3.51 (s, 2H), 2.90 (s, 3H), 2.68 (t, J=5.8 Hz, 2H), 2.65-2.59 (m, 2H), 2.14 (dtd, J=16.8, 11.3, 5.4 Hz, 4H), 1.75-1.62 (m, 2H).

### Example 529: 2-(3-(3-((4-methyl-4H-1,2,4-triazol-3-yl)methyl)oxetan-3-yl)phenyl)-6-((THF-2-yl)methyl)-4-(trifluoromethyl)isoindolin-1-one

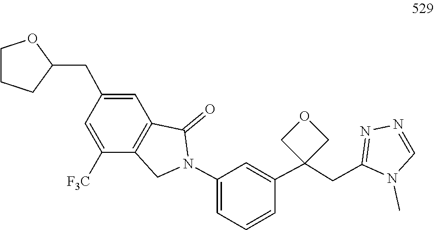 [SMILES: Cn1cnnc1CC1(c2cccc(N3Cc4c(cc(CC5CCCO5)cc4C(F)(F)F)C3=O)c2)COC1]

A solution of Example Y, step 3, (138 mg, 0.27 mmol), bis[(2-diphenylphosphino)-phenyl] ether (5.9 mg, 0.011 mmol), tris(dibenzylideneacetone)dipalladium(0) (5.0 mg, 0.005 mmol), sodium t-butoxide (52 mg, 0.54 mmol), and 4-penten-1-ol (23 mg, 0.27 mmol) in THF (1.1 ml) was stirred at 75° C. for 7 hr. After following General Work-up Procedure 1, the crude residue was purified Chromatography C to provide the title compound as a white solid (6 mg, 0.01 mmol). LCMS: C₂₇H₂₇F₃N₄O₃ requires 512.2, found 513.4 [M+H]⁺. ¹H NMR (500 MHz, Acetonitrile-d₃) δ 7.98-7.88 (m, 3H), 7.84 (s, 1H), 7.37 (t, J=8.0 Hz, 1H), 7.33 (t, J=2.0 Hz, 1H), 6.82-6.75 (m, 1H), 5.04 (d, J=6.1 Hz, 2H), 4.99 (d, J=6.1 Hz, 2H), 4.96 (s, 2H), 4.10 (qd, J=7.5, 5.0 Hz, 1H), 3.89-3.81 (m, 1H), 3.69 (td, J=7.9, 6.4 Hz, 1H), 3.58-3.49 (m, 2H), 3.03 (dd, J=13.9, 4.8 Hz, 1H), 2.97 (dd, J=13.9, 7.9 Hz, 1H), 2.88 (s, 3H), 2.05-1.99 (m, 1H), 1.93-1.83 (m, 2H), 1.60 (ddt, J=12.0, 8.7, 7.4 Hz, 1H).

### Example 530: 2-(3-(3-((4-methyl-4H-1,2,4-triazol-3-yl)methyl)oxetan-3-yl)phenyl)-6-((4-oxopiperidin-3-yl)methyl)-4-(trifluoromethyl)isoindolin-1-one

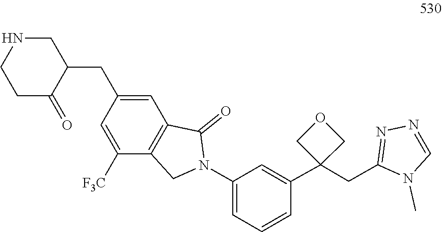 [SMILES: Cn1cnnc1CC1(c2cccc(N3Cc4c(cc(CC5CNCCC5=O)cc4C(F)(F)F)C3=O)c2)COC1]

Step 1: Synthesis of benzyl (E)-3-((2-(3-(3-((4-methyl-4H-1,2,4-triazol-3-yl)methyl)oxetan-3-yl)phenyl)-3-oxo-7-(trifluoromethyl)isoindolin-5-yl)methylene)-4-oxopiperidine-1-carboxylate. Benzyl 4-oxopiperidine-1-carboxylate (200 mg, 0.86 mmol) was combined with pyrrolidine (0.06 g, 0.86 mmol) in toluene (3.5 mL) and heated to 100° C. for 2 hr. The mixture was cooled to RT and Example Z (300 mg, 0.66 mmol) was added before heating 2 hr. at 100° C. After following General Work-up Procedure 1, the crude residue was purified using Chromatography B to afford the title compound (100 mg, 0.11 mmol).

Step 2: Synthesis of 2-(3-(3-((4-methyl-4H-1,2,4-triazol-3-yl)methyl)oxetan-3-yl)phenyl)-6-((4-oxopiperidin-3-yl)methyl)-4-(trifluoromethyl)isoindolin-1-one. To a vial containing benzyl (E)-3-((2-(3-(3-((4-methyl-4H-1,2,4-triazol-3-yl)methyl)oxetan-3-yl)phenyl)-3-oxo-7-(trifluoromethyl)isoindolin-5-yl)methylene)-4-oxopiperidine-1-carboxylate (100 mg, 0.15 mmol) was added 10% palladium on carbon (10 mg) followed by methanol (4 mL). Hydrogen gas was bubbled through the mixture for 10 min with stirring, before continuing to stir for 3 hr under hydrogen atmosphere. The reaction was purged with nitrogen gas for 15 min and then filtered through a pad of Celite, rinsing with dichloromethane and methanol. The title compound was obtained after purification on silica gel (1 to 40% methanol in dichloromethane with 0.1% ammonium hydroxide additive) as a yellow foam. LCMS: C₂₈H₂₈F₃N₅O₃ requires 539.2, found 540.5 [M+H]⁺. ¹H NMR (500 MHz, Acetonitrile-d₃) δ 7.88 (d, J=3.9 Hz, 3H), 7.79 (s, 1H), 7.34 (t, J=8.0 Hz, 1H), 7.29 (q, J=2.0 Hz, 1H), 6.76 (d, J=7.8 Hz, 1H), 5.01 (d, J=6.1 Hz, 2H), 4.96 (d, J=6.1 Hz, 2H), 4.93 (s, 2H), 3.52 (s, 2H), 3.35-3.14 (m, 3H), 2.91-2.76 (m, 5H), 2.62 (dd, J=14.2, 7.2 Hz, 1H), 2.54 (dd, J=12.6, 10.5 Hz, 1H), 2.49-2.37 (m, 1H), 2.27 (dt, J=13.3, 3.1 Hz, 1H).

### Example 531: 6-((l-methyl-4-oxopiperidin-3-yl)methyl)-2-(3-(3-((4-methyl-4H-1,2,4-triazol-3-yl)methyl)oxetan-3-yl)phenyl)-4-(trifluoromethyl)isoindolin-1-one

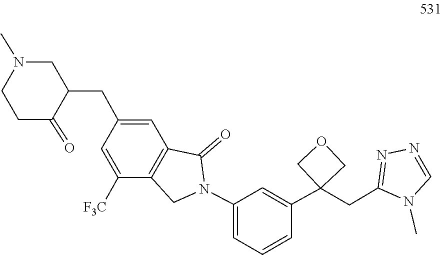 [SMILES: CN1CCC(=O)C(Cc2cc3c(c(C(F)(F)F)c2)CN(c2cccc(C4(Cc5nncn5C)COC4)c2)C3=O)C1]

Step 1: Synthesis of (E)-6-((l-methyl-4-oxopiperidin-3-ylidene)methyl)-2-(3-(3-((4-methyl-4H-1,2,4-triazol-3-yl)methyl)oxetan-3-yl)phenyl)-4-(trifluoromethyl)isoindolin-1-one. The reaction was carried out in a similar fashion to Example 530, Step 1, using Example Z (958 mg, 2.1 mmol) and 1-methylpiperidin-4-one (237 mg, 2.10 mmol) to provide the title compound as a yellow foam (240 mg).

Step 2: Synthesis of 6-((l-methyl-4-oxopiperidin-3-yl)methyl)-2-(3-(3-((4-methyl-4H-1,2,4-triazol-3-yl)methyl)oxetan-3-yl)phenyl)-4-(trifluoromethyl)isoindolin-1-one. The reaction was carried out in a similar fashion to Example 530, Step 2, using (E)-6-((l-methyl-4-oxopiperidin-3-ylidene)methyl)-2-(3-(3-((4-methyl-4H-1,2,4-triazol-3-yl)methyl)oxetan-3-yl)phenyl)-4-(trifluoromethyl)isoindolin-1-one (240 mg, 0.44 mmol) and reacting under hydrogen for 24 hr. The title compound was obtained after Chromatography B as a white solid. LCMS: C₂₈H₂₈F₃N₅O₃ requires 553.2, found 554.5 [M+H]⁺. ¹H NMR (500 MHz, Chloroform-d) δ 7.88 (d, J=4.8 Hz, 2H), 7.71 (s, 1H), 7.53 (ddd, J=8.1, 2.3, 0.9 Hz, 1H), 7.50 (t, J=2.0 Hz, 1H), 7.34 (t, J=7.9 Hz, 1H), 6.64 (dt, J=8.0, 1.1 Hz, 1H), 5.16 (dd, J=6.1, 2.0 Hz, 2H), 5.09 (dd, J=6.2, 1.6 Hz, 2H), 4.92 (d, J=1.5 Hz, 2H), 3.61 (s, 2H), 3.44-3.36 (m, 1H), 3.08 (ddt, J=12.6, 6.9, 3.4 Hz, 1H), 2.95 (tdd, J=8.8, 5.7, 2.0 Hz, 2H), 2.89 (s, 3H), 2.76-2.63 (m, 2H), 2.54-2.41 (m, 2H), 2.37 (s, 3H), 2.24-2.12 (m, 1H).

### Example 532 and 533: 6-(((cis)-4-hydroxy-1-methylpiperidin-3-yl)methyl)-2-(3-(3-((4-methyl-4H-1,2,4-triazol-3-yl)methyl)oxetan-3-yl)phenyl)-4-(trifluoromethyl)isoindolin-1-one and 6-(((3S,4S)-4-hydroxy-1-methylpiperidin-3-yl)methyl)-2-(3-(3-((4-methyl-4H-1,2,4-triazol-3-yl)methyl)oxetan-3-yl)phenyl)-4-(trifluoromethyl)isoindolin-1-one

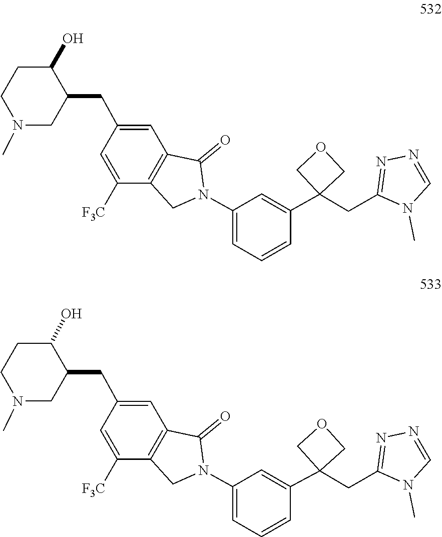 [SMILES: CN1CC[C@@H](O)[C@@H](Cc2cc3c(c(C(F)(F)F)c2)CN(c2cccc(C4(Cc5nncn5C)COC4)c2)C3=O)C1 ; CN1CC[C@H](O)[C@@H](Cc2cc3c(c(C(F)(F)F)c2)CN(c2cccc(C4(Cc5nncn5C)COC4)c2)C3=O)C1]

The 6-((1-methyl-4-oxopiperidin-3-yl)methyl)-2-(3-(3-((4-methyl-4H-1,2,4-triazol-3-yl)methyl)oxetan-3-yl)phenyl)-4-(trifluoromethyl)isoindolin-1-one (530, 45 mg, 0.08 mmol) was dissolved in ethanol (1.0 mL) before the addition of sodium borohydride (4.6 mg, 0.12 mmol). The reaction was stirred for 1 hr. before following General Work-up Procedure 1, the crude residue was purified using Chromatography C to afford separable racemic mixtures of the title compounds, PEAK 1-LCMS: C₂₈H₂₈F₃N₅O₃ requires 555.2, found 556.5 [M+H]⁺. ¹H NMR (500 MHz, Acetonitrile-d₃) δ 7.91-7.86 (m, 2H), 7.83 (s, 1H), 7.75 (s, 1H), 7.34 (t, J=8.0 Hz, 1H), 7.31 (t, J=2.0 Hz, 1H), 6.75 (dt, J=7.8, 1.2 Hz, 1H), 5.01 (d, J=6.0 Hz, 2H), 4.96 (d, J=6.1 Hz, 2H), 4.93 (s, 2H), 3.52 (s, 2H), 3.26 (dd, J=13.8, 4.0 Hz, 1H), 3.18 (td, J=9.7, 4.5 Hz, 1H), 2.85 (s, 3H), 2.71-2.64 (m, 1H), 2.56 (dd, J=13.7, 9.4 Hz, 1H), 2.50 (ddd, J=11.4, 3.8, 2.0 Hz, 1H), 2.08 (s, 3H), 1.93-1.73 (m, 4H), 1.64 (t, J=10.9 Hz, 1H), 1.51 (tdd, J=12.0, 10.1, 4.1 Hz, 1H) and PEAK 2-LCMS: C₂₈H28F₃N₅O₃ requires 555.2, found 556.5 [M+H]⁺. ¹H NMR (500 MHz, Acetonitrile-d₃) δ 7.94-7.71 (m, 4H), 7.40-7.25 (m, 2H), 6.75 (dt, J=7.9, 1.3 Hz, 1H), 5.03-4.90 (m, 6H), 3.64 (q, J=3.8 Hz, 1H), 3.52 (s, 2H), 2.91 (dd, J=13.5, 7.0 Hz, 1H), 2.85 (s, 3H), 2.80-2.44 (m, 3H), 2.15 (s, 3H), 2.01-1.96 (m, 1H), 1.84-1.73 (m, 1H), 1.66 (dt, J=9.1, 3.9 Hz, 2H).

### Example 534a and 534b: 2-cyclopropyl-6-methyl-N-{3-[(3S)-3-[(4-methyl-1,2,4-triazol-3-yl)methyl]oxolan-3-yl]phenyl}pyrimidine-4-carboxamide and 2-cyclopropyl-6-methyl-N-{3-[(3R)-3-[(4-methyl-1,2,4-triazol-3-yl)methyl]oxolan-3-yl]phenyl}pyrimidine-4-carboxamide

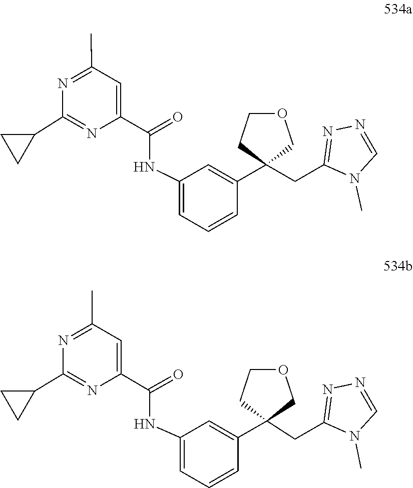 [SMILES: Cc1cc(C(=O)Nc2cccc([C@]3(Cc4nncn4C)CCOC3)c2)nc(C2CC2)n1 ; Cc1cc(C(=O)Nc2cccc([C@@]3(Cc4nncn4C)CCOC3)c2)nc(C2CC2)n1]

3-(3-((4-methyl-4H-1,2,4-triazol-3-yl)methyl)THF-3-yl)aniline (75 mg, 0.30 mmol) and 2-cyclopropyl-6-methylpyrimidine-4-carboxylic acid (54 mg, 0.30 mmol, were coupled in a similar fashion as for 74 to afford racemic title compound which was resolved by SFC over an AZ column with MeOH/CO₂ to afford: 34 mg (36%) of first peak: 2-cyclopropyl-6-methyl-N-{3-[(3S)-3-[(4-methyl-1,2,4-triazol-3-yl)methyl]oxolan-3-yl]phenyl}pyrimidine-4-carboxamide: ¹H NMR (500 MHz, Acetonitrile-d₃) δ 9.88 (s, 1H), 7.86 (s, 1H), 7.78-7.69 (m, 2H), 7.39 (t, J=2.0 Hz, 1H), 7.26 (t, J=7.9 Hz, 1H), 6.76 (dt, J=7.9, 1.3 Hz, 1H), 4.39 (d, J=8.4 Hz, 1H), 4.10 (q, J=8.0 Hz, 1H), 3.99-3.91 (m, 1H), 3.82 (d, J=8.4 Hz, 1H), 3.09 (s, 2H), 2.79 (s, 3H), 2.65 (ddd, J=12.3, 7.9, 4.3 Hz, 1H), 2.52 (s, 3H), 2.31 (tt, J=8.1, 4.7 Hz, 1H), 2.24 (ddd, J=12.4, 9.4, 8.1 Hz, 1H), 1.22-1.15 (m, 2H), 1.15-1.05 (m, 3H); LCMS: C₂₃H₂₆N₆O₂ requires: 418, found: m/z=419 [M+H]⁺ and 34 mg (36%) of second peak, with identical ¹H NMR and LCMS spectra.

### Example 535: 6-{5-azaspiro[2.4]heptan-5-ylmethyl}-2-(3-{3-[(4-methyl-1,2-oxazol-3-yl)methyl]oxetan-3-yl}phenyl)-4-(trifluoromethyl)-3H-isoindol-1-one

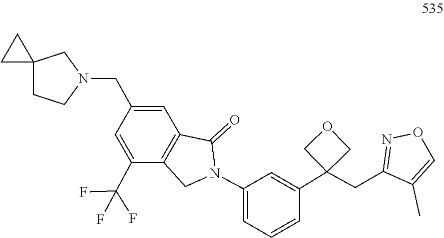 [SMILES: Cc1conc1CC1(c2cccc(N3Cc4c(cc(CN5CCC6(CC6)C5)cc4C(F)(F)F)C3=O)c2)COC1]

Step 1. Synthesis of 2-(3-{3-[(4-methyl-1,2-oxazol-3-yl)methyl]oxetan-3-yl}phenyl)-3-oxo-7-(trifluoromethyl)-1H-isoindole-5-carbaldehyde. The condensation between 3 3-{3-[(4-methyl-1,2-oxazol-3-yl)methyl]oxetan-3-yl}aniline (46 mg, 0.19 mmol) and methyl 2-(bromomethyl)-5-formyl-3-(trifluoromethyl)benzoate (64 mg, 0.20 mmol) was carried out according to the procedure from Example 260 step 2 to afford 51 mg (60%) of the title compound as a yellow solid.

Step 2. Synthesis of 6-{5-azaspiro[2.4]heptan-5-ylmethyl}-2-(3-{3-[(4-methyl-1,2-oxazol-3-yl)methyl]oxetan-3-yl}phenyl)-4-(trifluoromethyl)-3H-isoindol-1-one. 2-(3-{3-[(4-methyl-1,2-oxazol-3-yl)methyl]oxetan-3-yl}phenyl)-3-oxo-7-(trifluoromethyl)-1H-isoindole-5-carbaldehyde (50 mg, 0.11 mmol) and 5-azaspiro[2.4]heptane hydrochloride (44 mg, 0.33 mmol) were coupled in a similar fashion as for 447, step 4 to afford the title compound as an off-white solid: ¹H NMR (500 MHz, DMSO-d₆) δ 8.41 (s, 1H), 7.99 (s, 1H), 7.94 (s, 1H), 7.83 (dd, J=8.1, 2.1 Hz, 1H), 7.52 (t, J=1.9 Hz, 1H), 7.36 (t, J=7.9 Hz, 1H), 6.84 (d, J=7.7 Hz, 1H), 5.12 (s, 2H), 4.93-4.79 (m, 4H), 3.80 (s, 2H), 3.38 (s, 2H), 2.70 (t, 7=6.8 Hz, 2H), 2.45 (s, 2H), 1.77 (t, J=6.8 Hz, 2H), 1.31 (d, J=1.0 Hz, 3H), 0.50 (d, 7=4.8 Hz, 4H); LCMS: C₃₀H₃₀F₃N₃O₃ requires: 537, found: m/z=538 [M+H]⁺.

### Example 536: 6-((3-fluoro-2,2-dimethylazetidin-1-yl)methyl)-2-(3-(3-((4-methyl-4H-1,2,4-triazol-3-yl)methyl)oxetan-3-yl)phenyl)-4-(trifluoromethyl)isoindolin-1-one

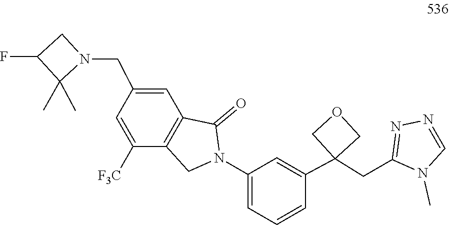 [SMILES: Cn1cnnc1CC1(c2cccc(N3Cc4c(cc(CN5CC(F)C5(C)C)cc4C(F)(F)F)C3=O)c2)COC1]

Step 1: Synthesis of l-benzhydryl-3-fluoro-2,2-dimethylazetidine. 1-Benzhydryl-2,2-dimethylazetidin-3-ol (100 mg, 0.37 mmol) was dissolved in DCM (2 mL) at 0° C. Deoxo-Fluor (2.7 M in DCM, 0.21 mL, 0.56 mmol) was added slowly and the reaction was gradually warmed to ambient temperature. Sat. NaHCO₃ was added and the product was extracted with DCM (×3). The combined organic layer was dried. After concentration, the crude was purified by chromatography B to afford 98 mg (98%) yield of the title compound.

Step 2: Synthesis of 3-fluoro-2,2-dimethylazetidine. Ammonium formate (76 mg, 1.2 mmol) in methanol (1 mL) was added to1-benzhydryl-3-fluoro-2,2-dimethylazetidine (98 mg, 0.36 mmol) at ambient temperature. Palladium on carbon (10%, wet, 8.8 mg) was added and the reaction was heated at 80° C. for 3 hours. Heat was reduced to 64° C. and more ammonium formate (55 mg, 0.87 mmol) was added. After the disappearance of the starting material the reaction was cooled to ambient temperature. Palladium on carbon was filtered off and sat. NaHCO₃ was added. The product was extracted with DCM (×3) followed by chloroform: isopropyl alcohol (2:1) (×3). The combined organic layer was dried, and concentrated to 2 mL and used as is in the next step.

Step 3: Synthesis of 6-((3-fluoro-2,2-dimethylazetidin-1-yl)methyl)-2-(3-(3-((4-methyl-4H-1,2,4-triazol-3-yl)methyl)oxetan-3-yl)phenyl)-4-(trifluoromethyl)isoindolin-1-one. To the 3-fluoro-2,2-dimethylazetidine (<0.36 mmol) solution was added Example Z (31 mg, 0.067 mmol) at ambient temperature. After stirring for 5 minutes, NaBH(OAc)₃ (30 mg, 0.14 mmol) was added and the reaction was stirred overnight. Only trace amount of desired product was observed by LCMS. After removal of solvent, methanol (2 mL) and acetic acid (4 μL) was added to the reaction. Palladium on carbon (10%, wet, 8.5 mg) was added and the reaction was stirred under hydrogen balloon overnight. After filtering off palladium, the reaction was evaporated and re-suspended in a mixture of methanol and DCM. More Example Z (13 mg, 0.029 mmol) and NaHB(OAc)₃ (13 mg, 0.059 mmol) was added and stirring continued at ambient temperature for 2 hours. The mixture was purified by chromatography B. The product was further purified by reverse phase HPLC to give 1.9 mg of 6-((3-fluoro-2,2-dimethylazetidin-1-yl)methyl)-2-(3-(3-((4-methyl-4H-1,2,4-triazol-3-yl)methyl)oxetan-3-yl)phenyl)-4-(trifluoromethyl)isoindolin-1-one (3.4% yield). ¹H NMR (500 MHz, Methanol-d₄) δ 8.19 (s, 1H), 8.06 (s, 1H), 7.96 (d, J=1.4 Hz, 1H), 7.76 (ddd, J=8.1, 2.2, 0.9 Hz, 1H), 7.43-7.36 (m, 2H), 6.74 (ddd, J=7.7, 1.8, 0.9 Hz, 1H), 5.13-5.01 (m, 6H), 4.77 (ddd, J=57.9, 6.0, 4.8 Hz, 1H), 3.89 (d, J=13.8 Hz, 1H), 3.77 (d, J=13.7 Hz, 1H), 3.65 (s, 2H), 3.52 (ddd, J=14.0, 8.3, 5.9 Hz, 1H), 3.20 (ddd, J=21.6, 8.3, 4.7 Hz, 1H), 2.89 (s, 3H), 1.32-1.21 (m, 6H); LCMS: C₂₈H₂₉F₄N₅O₂ requires: 543, found: m/z=544 [M+H]⁺.

### Example 537: 3-methyl-1-((2-(3-(3-((4-methyl-4H-1,2,4-triazol-3-yl)methyl)oxetan-3-yl)phenyl)-3-oxo-7-(trifluoromethyl)isoindolin-5-yl)methyl)azetidine-3-carboxylic acid

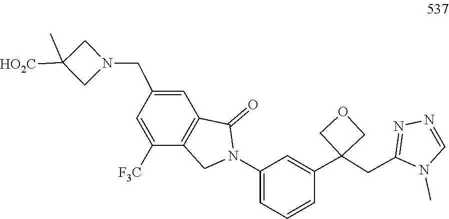 [SMILES: Cn1cnnc1CC1(c2cccc(N3Cc4c(cc(CN5CC(C)(C(=O)O)C5)cc4C(F)(F)F)C3=O)c2)COC1]

The reductive amination was carried out in a similar fashion as for 447, step 4 using 2-(3-{3-[(4-methyl-4H-1,2,4-triazol-3-yl)methyl]oxetan-3-yl}phenyl)-3-oxo-7-(trifluoromethyl)-2,3-dihydro-1H-isoindole-5-carbaldehyde (33 mg, 0.072 mmol) and 3-methylazetidine-3-carboxylic acid (28 mg, 0.25 mmol) as reactants to afford the title compound (11 mg, 23%) as a white solid. ¹H NMR (500 MHz, DMSO-d₆) δ 10.45 (s, 1H), 8.34 (s, 1H), 8.26 (d, J=10.1 Hz, 1H), 8.19 (s, 1H), 7.88 (d, J=8.2 Hz, 1H), 7.42 (s, 1H), 7.37 (t, J=8.0 Hz, 1H), 6.83 (d, J=7.7 Hz, 1H), 5.16 (s, 2H), 4.95 (d, J=6.1 Hz, 2H), 4.88 (d, J=6.1 Hz, 2H), 4.61 (s, 2H), 4.43 (m, 1H), 4.21 (d, J=41.9 Hz, 2H), 3.55 (s, 2H), 2.96 (s, 3H), 1.51 (s, 3H); LCMS: C₂₈H₂₈F₃N₅O₄ requires: 555, found: m/z=556 [M+H]⁺.

### Example 538: 2-cyclopropyl-6-(1-(3-fluoroazetidin-1-yl)ethyl)-N-(3-(3-((4-methyl-4H-1,2,4-triazol-3-yl)methyl)oxetan-3-yl)phenyl)pyrimidine-4-carboxamide

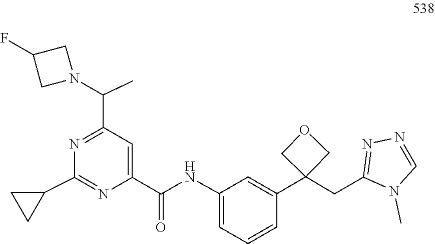 [SMILES: CC(c1cc(C(=O)Nc2cccc(C3(Cc4nncn4C)COC3)c2)nc(C2CC2)n1)N1CC(F)C1]

Step 1: Synthesis of ethyl 2-cyclopropyl-6-hydroxypyrimidine-4-carboxylate: To a solution of diethyl oxalacetate sodium salt (20.0 g, 106.28 mmol) in EtOH (200 mL) were added K₂CO₃ (36.7 g, 265.70 mmol) and cyclopropanecarboximidamide (8.9 g, 106.28 mmol). The resulting solution was stirred at 80° C. for 4 h before being concentrated. The residue was purified by chromatography B to afford the title compound (10.0 g, 45%) as a yellow solid. MS (ESI) calculated for (C₁₀H₁₂N₂O₃) [M+H]⁺, 209.1; found, 209.0.

Step 2: Synthesis of ethyl 6-chloro-2-cyclopropylpyrimidine-4-carboxylate: A mixture of ethyl 2-ethyl-6-hydroxypyrimidine-4-carboxylate (6.0 g, 28.84 mmol) in phosphoroyl trichloride (100 mL) was stirred at 110° C. for 2 h before being concentrated. The resulting mixture was poured into cold water cautiously and then extracted with EtOAc. The combined organic layer was washed with brine, dried, filtered and concentrated. The crude was purified by chromatography A to afford the title compound (1.3858 g, 6% over two steps) as a light yellow semi-solid. MS (ESI) calculated for (C₁₀H₁₁ClN₂O₂) [M+H]⁺, 227.1; found, 227.2.

Step 3: Synthesis of ethyl 2-cyclopropyl-6-(l-ethoxyethenyl)pyrimidine-4-carboxylate: To a degassed solution of tributyl(l-ethoxyethenyl)stannane (841. mg, 2.33 mmol) in dioxane (10 mL) were added ethyl 6-chloro-2-cyclopropylpyrimidine-4-carboxylate (480 mg, 2.12 mmol) and Pd(PPh₃)₂Cl₂ (148 mg, 0.212 mmol). The resulting solution was stirred at 100° C. for 4 h. The reaction was quenched by the addition of potassium iodide solution and extracted with EtOAc. The combined organic layer was washed with brine, dried, filtered and concentrated. The crude was purified by chromatography A to afford the title compound (400 mg, 72%) as an off-white solid. MS (ESI) calculated for (C₁₄H₁₈N₂O₃) [M+H]⁺, 263.1; found, 263.0.

Step 4: Synthesis of ethyl 6-acetyl-2-cyclopropylpyrimidine-4-carboxylate: To a solution of ethyl 2-cyclopropyl-6-(l-ethoxyethenyl)pyrimidine-4-carboxylate (2.3 g, 8.768 mmol) in acetone (30 mL) was added pTsOH (1.51 g, 8.768 mmol). The resulting solution was stirred at 60° C. for 4 h. The reaction was concentrated and purified by chromatography A to afford the title compound (932.9 mg, 45%) as a white solid. MS (ESI) calculated for (C₁₂H₁₄N₂O₃) [M+H]⁺, 235.1; found, 235.0.

Step 5: Synthesis of 6-acetyl-2-cyclopropylpyrimidine-4-carboxylic acid. Ethyl 6-acetyl-2-cyclopropylpyrimidine-4-carboxylate (100 mg, 0.43 mmol) was dissolved in THF (1.2 mL) at ambient temperature. Lithium hydroxide (1M in water, 0.43 mL, 0.43 mmol) was added and the reaction was stirred for 1 h. More lithium hydroxide (1M in water, 30 μL, 0.030 mmol) was added and the hydrolysis was complete. After removal of THF the mixture was diluted with water and lyophilized to give the title compound as a lithium salt.

Step 6: Synthesis of 2-cyclopropyl-6-(1-(3-fluoroazetidin-1-yl)ethyl)pyrimidine-4-carboxylic acid. Crude lithium salt of 6-acetyl-2-cyclopropylpyrimidine-4-carboxylic acid (27 mg, <0.13 mmol) and 3-fluoroazetidine hydrochloride (28 mg, 0.25 mmol) was dissolved in MeOH (0.5 mL) at ambient temperature. The mixture was microwaved at 100° C. for 1 min. NaBH₃CN (13 mg, 0.20 mmol) was added and the reaction was microwaved at 80° C. for 30 minutes. 1N-HCl was added and the mixture was washed with DCM Sat. NaHCO₃ was added until the mixture was neutral followed by washing with DCM (×3). The remaining aqueous layer was purified by reverse phase HPLC to give the desired product in 5.5 mg.

Step 7: Synthesis of 2-cyclopropyl-6-(1-(3-fluoroazetidin-1-yl)ethyl)-N-(3-(3-((4-methyl-4H-1,2,4-triazol-3-yl)methyl)oxetan-3-yl)phenyl)pyrimidine-4-carboxamide. The aminde bond was constructed in a similar fashion as to 74 using 2-cyclopropyl-6-(1-(3-fluoroazetidin-1-yl)ethyl)pyrimidine-4-carboxylic acid (5.5 mg, 0.018 mmol) and 3-(3-((4-methyl-4H-1,2,4-triazol-3-yl)methyl)oxetan-3-yl)aniline (9.5 mg, 0.039 mmol) to afford the title compound as a white solid (1.5 mg, 17% yield). ¹H NMR (500 MHz, Methanol-d4) δ 8.19 (s, 1H), 7.88 (s, 1H), 7.71 (ddd, J=8.2, 2.2, 0.9 Hz, 1H), 7.37-7.30 (m, 2H), 6.67 (dt, J=7.5, 1.3 Hz, 1H), 5.24-5.08 (m, 1H), 5.08-5.03 (m, 4H), 3.77-3.54 (m, 5H), 2.87 (s, 3H), 2.39 (tt, J=8.2, 4.7 Hz, 1H), 1.28 (d, J=6.7 Hz, 3H), 1.20 (ddd, J=51.9, 8.2, 4.1 Hz, 4H); LCMS: C₂₆H₃₀FN₇O₂ requires: 491, found: m/z=492 [M+H]⁺.

### Example 539: (S)-2-methyl-1-((2-(3-(3-((4-methyl-4H-1,2,4-triazol-3-yl)methyl)oxetan-3-yl)phenyl)-3-oxo-7-(trifluoromethyl)isoindolin-5-yl)methyl)pyrrolidine-2-carboxylic acid

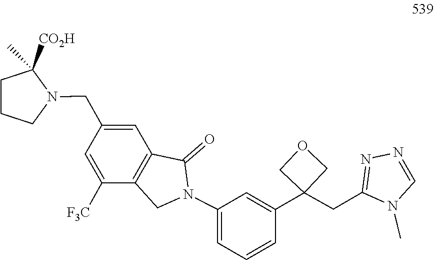 [SMILES: Cn1cnnc1CC1(c2cccc(N3Cc4c(cc(CN5CCC[C@@]5(C)C(=O)O)cc4C(F)(F)F)C3=O)c2)COC1]

The reductive amination was carried out in a similar fashion as for 447, step 4 using (R)-2-(3-(1-(4-methyl-4H-1,2,4-triazol-3-yl)propan-2-yl)phenyl)-3-oxo-7-(trifluoromethyl)-soindoline-5-carbaldehyde (30 mg, 0.065 mmol) and (S)-2-methylpyrrolidine-2-carboxylic acid (26 mg, 0.20 mmol) as reactants to afford the title compound (5.5 mg, 15% yield) as a white solid. ¹H NMR (500 MHz, DMSO-d6) δ 8.30 (s, 1H), 8.18 (s, 1H), 8.10 (s, 1H), 7.88 (dd, J=8.0, 2.2 Hz, 1H), 7.41 (t, J=2.0 Hz, 1H), 7.37 (t, J=8.0 Hz, 1H), 6.82 (d, J=8.3 Hz, 1H), 5.16 (s, 2H), 4.95 (d, J=6.1 Hz, 2H), 4.89 (d, J=6.0 Hz, 2H), 2.95 (s, 3H), 2.24-1.74 (m, 4H); LCMS: C₂₉H₃₀F₃N₅O₄ requires: 569, found: m/z=570 [M+H]⁺.

### Example 540: 6-(l-hydroxyethyl)-2-(3-(3-((4-methyl-4H-1,2,4-triazol-3-yl)methyl)oxetan-3-yl)phenyl)-4-(trifluoromethyl)isoindolin-1-one

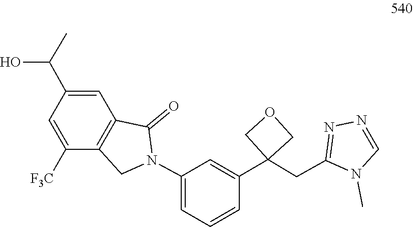 [SMILES: CC(O)c1cc2c(c(C(F)(F)F)c1)CN(c1cccc(C3(Cc4nncn4C)COC3)c1)C2=O]

6-Acetyl-2-(3-(3-((4-methyl-4H-1,2,4-triazol-3-yl)methyl)oxetan-3-yl)phenyl)-4-(trifluoromethyl)isoindolin-1-one (413, step 1, 107 mg, 0.23 mmol) was dissolved in MeOH (1 mL) at 0° C. NaBH₄ (17 mg, 0.45 mmol) was added and the reaction was slowly warmed to ambient temperature and stirred for one hour. Water and DCM were added to the reaction mixture and the product was extracted with DCM five times. The combined organic layer was dried and concentrated. The crude that solidified was washed with minimum amount of DCM and dried under vacuum to give the title compound as a white solid (110 mg, 0.23 mmol). ¹H NMR (500 MHz, DMSO-d6) δ 8.17 (s, 1H), 8.01 (d, J=1.3 Hz, 1H), 7.97 (s, 1H), 7.88 (ddd, J=8.3, 2.2, 0.9 Hz, 1H), 7.38 (t, J=2.0 Hz, 1H), 7.34 (t, J=7.9 Hz, 1H), 6.75 (dt, J=7.8, 1.1 Hz, 1H), 5.55 (d, J=4.6 Hz, 1H), 5.08 (s, 2H), 4.95 (d, J=6.1 Hz, 2H), 4.88 (d, J=6.1 Hz, 2H), 3.50 (s, 2H), 2.90 (s, 3H), 1.38 (d, J=6.4 Hz, 3H); LCMS: C₂₄H₂₃F₃N₄O₃ requires: 472, found: m/z=473 [M+H]⁺.

### Example 541a and 541b: 6-((1S)-1-(3-azabicyclo[3.1.0]hexan-3-yl)ethyl)-2-(3-(3-((4-methyl-4H-1,2,4-triazol-3-yl)methyl)oxetan-3-yl)phenyl)-4-(trifluoromethyl)isoindolin-1-one and 6-((1R)-1-(3-azabicyclo[3.1.0]hexan-3-yl)ethyl)-2-(3-(3-((4-methyl-4H-1,2,4-triazol-3-yl)methyl)oxetan-3-yl)phenyl)-4-(trifluoromethyl)isoindolin-1-one

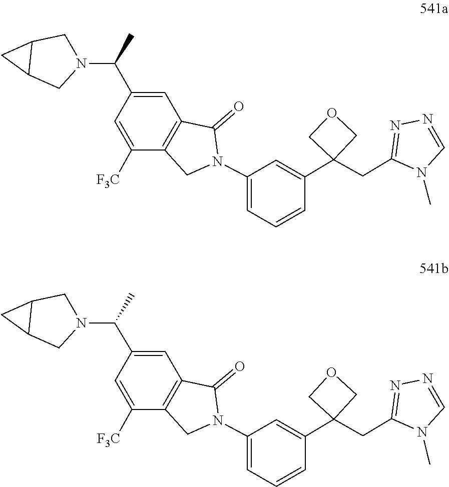 [SMILES: C[C@@H](c1cc2c(c(C(F)(F)F)c1)CN(c1cccc(C3(Cc4nncn4C)COC3)c1)C2=O)N1CC2CC2C1 ; C[C@H](c1cc2c(c(C(F)(F)F)c1)CN(c1cccc(C3(Cc4nncn4C)COC3)c1)C2=O)N1CC2CC2C1]

Step 1: Synthesis of 6-(1-(3-azabicyclo[3.1.0]hexan-3-yl)ethyl)-2-(3-(3-((4-methyl-4H-1,2,4-triazol-3-yl)methyl)oxetan-3-yl)phenyl)-4-(trifluoromethyl)isoindolin-1-one. Reductive amination of the methyl ketone was carried out according to the procedure of the procedure for 459, step 2 using (R)-6-acetyl-2-(3-(1-(4-methyl-4H-1,2,4-triazol-3-yl)propan-2-yl)phenyl)-4-(trifluoromethyl)isoindolin-1-one (130 mg, 0.28 mmol) and 3-azabicyclo[3.1.0]-hexane hydrochloride (100 mg, 0.84 mmol) as reactants to afford the title compound in 101 mg (66%) yield.

Step 2: 6-((1S)-1-(3-azabicyclo[3.1.0]hexan-3-yl)ethyl)-2-(3-(3-((4-methyl-4H-1,2,4-triazol-3-yl)methyl)oxetan-3-yl)phenyl)-4-(trifluoromethyl)isoindolin-1-one and 6-((1R)-1-(3-azabicyclo[3.1.0]hexan-3-yl)ethyl)-2-(3-(3-((4-methyl-4H-1,2,4-triazol-3-yl)-methyl)oxetan-3-yl)phenyl)-4-(trifluoromethyl)isoindolin-1-one. 6-(1-(3-azabicyclo[3.1.0]-hexan-3-yl)ethyl)-2-(3-(3-((4-methyl-4H-1,2,4-triazol-3-yl)methyl)oxetan-3-yl)phenyl)-4-(trifluoromethyl)isoindolin-1-one (100 mg) was separated using column IG with CO₂ and mixture of methanol:acetonitrile (7:3) with 0.1% diethylamine as mobile phase to afford 6-((1S)-1-(3-azabicyclo[3.1.0]hexan-3-yl)ethyl)-2-(3-(3-((4-methyl-4H-1,2,4-triazol-3-yl)methyl)oxetan-3-yl)phenyl)-4-(trifluoromethyl)isoindolin-1-one (43 mg) and 6-((1R)-1-(3-azabicyclo[3.1.0]-hexan-3-yl)ethyl)-2-(3-(3-((4-methyl-4H-1,2,4-triazol-3-yl)methyl)oxetan-3-yl)phenyl)-4-(trifluoromethyl)isoindolin-1-one (44 mg).

¹H NMR (500 MHz, DMSO-d6) δ 8.19 (s, 1H), 7.93 (d, J=1.3 Hz, 1H), 7.92-7.86 (m, 2H), 7.39 (t, J=2.0 Hz, 1H), 7.36 (t, J=8.0 Hz, 1H), 6.77 (dt, J=7.8, 1.2 Hz, 1H), 5.09 (s, 2H), 4.97 (d, J=6.0 Hz, 2H), 4.89 (d, J=6.1 Hz, 2H), 3.62 (q, J=6.6 Hz, 1H), 3.52 (s, 2H), 3.13 (d, J=8.5 Hz, 1H), 2.90 (s, 3H), 2.44 (dd, J=8.5, 3.7 Hz, 1H), 2.17 (dd, J=8.7, 3.7 Hz, 1H), 1.53-1.25 (m, 5H), 0.68 (q, J=3.7 Hz, 1H), 0.35 (td, J=7.6, 3.7 Hz, 1H); LCMS: C₂₉H₃₀F₃N₅O₂ requires: 537, found: m/z=538 [M+H]⁺.

¹H NMR (500 MHz, DMSO-d6) δ 8.19 (s, 1H), 7.93 (s, 1H), 7.88 (q, J=2.8 Hz, 2H), 7.39 (t, J=1.9 Hz, 1H), 7.36 (t, J=7.9 Hz, 1H), 6.77 (dt, J=7.9, 1.2 Hz, 1H), 5.09 (s, 2H), 4.97 (d, J=5.9 Hz, 2H), 4.89 (d, J=6.0 Hz, 2H), 3.62 (q, J=6.6 Hz, 1H), 3.52 (s, 2H), 3.13 (d, J=8.5 Hz, 1H), 2.90 (s, 3H), 2.44 (dd, J=8.4, 3.7 Hz, 1H), 2.17 (dd, J=8.7, 3.7 Hz, 1H), 1.47-1.25 (m, 5H), 0.68 (q, J=3.7 Hz, 1H), 0.35 (td, J=7.7, 3.7 Hz, 1H); LCMS: C₂₉H₃₀F₃N₅O₂ requires: 537, found: m/z=538 [M+H]⁺.

### Example 542: 6-(1-(1H-imidazol-1-yl)ethyl)-2-(3-(3-((4-methyl-4H-1,2,4-triazol-3-yl)methyl)oxetan-3-yl)phenyl)-4-(trifluoromethyl)isoindolin-1-one

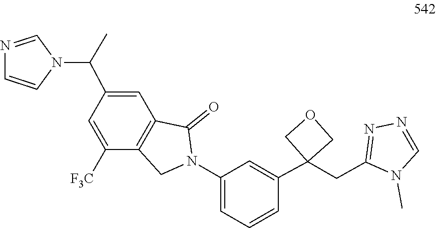 [SMILES: CC(c1cc2c(c(C(F)(F)F)c1)CN(c1cccc(C3(Cc4nncn4C)COC3)c1)C2=O)n1ccnc1]

6-(1-hydroxyethyl)-2-(3-(3-((4-methyl-4H-1,2,4-triazol-3-yl)methyl)oxetan-3-yl)phenyl)-4-(trifluoromethyl)isoindolin-1-one (13 mg, 0.027 mmol) and 1,1′-carbonyldiimidazol (8.7 mg, 0.054 mmol) were dissolved in NMP (0.5 mL) and microwaved at 170° C. for 3 hours. The reaction was diluted with MeOH and purified by reverse phase HPLC to give the title compound in 3.9 mg (28%) yield. ¹H NMR (500 MHz, DMSO-d6) δ 8.16 (s, 1H), 7.95 (d, J=1.4 Hz, 1H), 7.94-7.90 (m, 2H), 7.87 (ddd, J=8.1, 2.3, 1.0 Hz, 1H), 7.40 (t, J=1.2 Hz, 1H), 7.37-7.29 (m, 2H), 6.94 (t, J=1.1 Hz, 1H), 6.77 (dt, J=7.5, 1.3 Hz, 1H), 5.81 (q, J=7.1 Hz, 1H), 5.07 (s, 2H), 4.94 (d, J=6.0 Hz, 2H), 4.87 (d, J=6.1 Hz, 2H), 3.49 (s, 2H), 2.89 (s, 3H), 1.89 (d, J=7.1 Hz, 3H); LCMS: C₂₇H₂₅F₃N₆O₂ requires: 522, found: m/z=523 [M+H]⁺.

### Example 543a and 543b: (S)-6-(1-(3-fluoro-3-methylazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one and (R)-6-(1-(3-fluoro-3-methylazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one

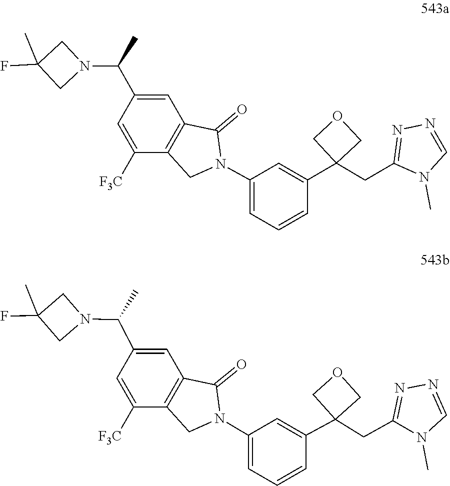 [SMILES: C[C@@H](c1cc2c(c(C(F)(F)F)c1)CN(c1cccc(C3(Cc4nncn4C)COC3)c1)C2=O)N1CC(C)(F)C1 ; C[C@H](c1cc2c(c(C(F)(F)F)c1)CN(c1cccc(C3(Cc4nncn4C)COC3)c1)C2=O)N1CC(C)(F)C1]

Step 1: Synthesis of 6-(1-(3-fluoro-3-methylazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one. Reductive amination of the methyl ketone was carried out according to the procedure of the procedure for 459, step 2 using (R)-6-acetyl-2-(3-(1-(4-methyl-4H-1,2,4-triazol-3-yl)propan-2-yl)phenyl)-4-(trifluoromethyl)isoindolin-1-one (43 mg, 0.092 mmol) and 3-fluoro-3-methyl-azetidine hydrochloride (36 mg, 0.29 mmol) as reactants to afford the title compound in 35 mg (70%) yield.

Step 2: Synthesis of (S)-6-(1-(3-fluoro-3-methylazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one and (R)-6-(1-(3-fluoro-3-methylazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one. 6-(1-(3-fluoro-3-methylazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one (30 mg) was separated using column OZ followed by (7?, 7?) Whelk-O with CO₂ and mixture of methanol:acetonitrile (7:3) with 0.1% diethylamine as mobile phase to afford (S)-6-(1-(3-fluoro-3-methylazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one (12 mg) and (R)-6-(l-(3-fluoro-3-methylazetidin-1-yl)ethyl)-2-(3-(3-((4-methyl-4H-1,2,4-triazol-3-yl)methyl)-oxetan-3-yl)phenyl)-4-(trifluoromethyl)isoindolin-1-one (11 mg).

¹H NMR (500 MHz, DMSO-d6) δ 8.17 (s, 1H), 7.98 (s, 1H), 7.92 (s, 1H), 7.86 (ddd, J=8.3, 2.2, 1.0 Hz, 1H), 7.37 (t, J=2.0 Hz, 1H), 7.34 (t, J=8.0 Hz, 1H), 6.76 (dt, J=7.9, 1.3 Hz, 1H), 5.07 (s, 2H), 4.95 (d, J=6.0 Hz, 2H), 4.87 (d, J=6.0 Hz, 2H), 3.69 (q, J=6.5 Hz, 1H), 3.50 (s, 2H), 3.43-3.04 (m, 4H), 2.88 (s, 3H), 1.53 (d, J=22.4 Hz, 3H), 1.19 (d, J=6.5 Hz, 3H); LCMS: C₂₈H₂₉F₄N₅O₂ requires: 543, found: m/z=544 [M+H]⁺.

¹H NMR (500 MHz, DMSO-d6) δ 8.17 (s, 1H), 7.98 (s, 1H), 7.92 (s, 1H), 7.86 (ddd, J=8.3, 2.2, 1.0 Hz, 1H), 7.37 (t, J=2.0 Hz, 1H), 7.34 (t, J=8.0 Hz, 1H), 6.76 (dt, J=7.9, 1.3 Hz, 1H), 5.07 (s, 2H), 4.95 (d, J=6.0 Hz, 2H), 4.87 (d, J=6.0 Hz, 2H), 3.69 (q, J=6.5 Hz, 1H), 3.50 (s, 2H), 3.43-3.04 (m, 4H), 2.88 (s, 3H), 1.53 (d, J=22.4 Hz, 3H), 1.19 (d, J=6.5 Hz, 3H); LCMS: C₂₈H₂₉F₄N₅O₂ requires: 543, found: m/z=544 [M+H]⁺.

### Example 544: 2,2-difluoro-2-(2-(3-(3-((4-methyl-4H-1,2,4-triazol-3-yl)methyl)oxetan-3-yl)phenyl)-3-oxo-7-(trifluoromethyl)isoindolin-5-yl)acetic acid

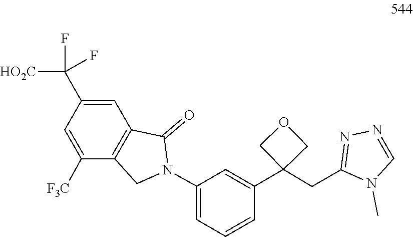 [SMILES: Cn1cnnc1CC1(c2cccc(N3Cc4c(cc(C(F)(F)C(=O)O)cc4C(F)(F)F)C3=O)c2)COC1]

Step 1: Synthesis of methyl 5-iodo-2-methyl-3-(trifluoromethyl)benzoate: To a solution of methyl 2-methyl-3-(trifluoromethyl)benzoate (23.4 g, crude) in TfOH (200 mL) was added NIS (29 g, 0.13 mol) in portions at 10-20° C. Then the mixture was heated at 60° C. for 16 h. The mixture was diluted with water and extracted with EtOAc. The combined organic layer was washed with water, NaHCO₃ (sat.) and Na₂S₂O₃ (sat.), dried, filtered and concentrated. The residue was purified by chromatography A to afford the title compound (12.4 g, 36% over two steps) as a yellow oil. ¹H NMR (400 MHz, CDCl₃) δ 8.26 (d, J=2.0 Hz, 1H), 8.08 (d, J=2.0 Hz, 1H), 3.95 (s, 3H), 2.60 (s, 3H).

Step 2: Synthesis of methyl 5-(2-ethoxy-1,1-difluoro-2-oxoethyl)-2-methyl-3-(trifluoromethyl)benzoate: A mixture of methyl 5-iodo-2-methyl-3-(trifluoromethyl)benzoate (12.4 g, 36.0 mmol), ethyl 2-bromo-2,2-difluoroacetate (18.2 g, 89.6 mmol) and Cu powder (11.4 g, 179 mmol) in DMSO (120 mL) was heated at 60° C. for 24 h. The mixture was diluted with sat. NH₄Cl and extracted with EtOAc. The combined organic layer was dried, filtered and concentrated. The residue was purified by chromatography A to afford the title compound (10.6 g, 86%) as a yellow oil.

Step 3: Synthesis of methyl 2-(bromomethyl)-5-(2-ethoxy-1,1-difluoro-2-oxoethyl)-3-(trifluoromethyl)benzoate: A mixture of methyl 5-(2-ethoxy-1,1-difluoro-2-oxoethyl)-2-methyl-3-(trifluoromethyl)benzoate (10.6 g, 30.9 mmol), NBS (8.3 g, 46.7 mmol) and BPO (2.2 g, 9.4 mmol) in CCl₄ (150 mL) was heated at 80° C. for 16 h. The solids were filtered out and the filtrate was concentrated. The residue was purified by chromatography A to afford the title compound (5.2 g, 40%) as a light yellow oil.

Step 4: Synthesis of 2,2-difluoro-2-(2-(3-(3-((4-methyl-4H-1,2,4-triazol-3-yl)methyl)oxetan-3-yl)phenyl)-3-oxo-7-(trifluoromethyl)isoindolin-5-yl)acetic acid. The indolone formation reaction was carried out in a manner similar to 260, step 2 using 3-(3-((4-methyl-4H-1,2,4-triazol-3-yl)methyl)oxetan-3-yl)aniline (150 mg, 0.63 mmol) and methyl 2-(bromomethyl)-5-(2-ethoxy-1,1-difluoro-2-oxoethyl)-3-(trifluoromethyl)benzoate (260 mg, 0.63 mmol) as reactants. The product partially hydrolyzed in the reaction mixture. After reverse phase HPLC purification, the title compound was obtained in 2.7 mg (2.5%) yield. ¹H NMR (500 MHz, DMSO-d6) δ 8.27 (s, 1H), 8.12 (d, J=10.0 Hz, 2H), 7.93-7.85 (m, 1H), 7.44 (t, J=2.0 Hz, 1H), 7.37 (t, J=8.0 Hz, 1H), 6.80 (dd, J=7.6, 1.6 Hz, 1H), 5.20 (s, 2H), 4.96 (d, J=6.0 Hz, 2H), 4.89 (d, J=6.1 Hz, 2H), 3.54 (s, 2H), 2.93 (s, 3H); LCMS: C₂₄H₁₉F₅N₄O₄ requires: 522, found: m/z=523 [M+H]⁺.

### Example 545 and 546: 6-((S)-1-((S)-3-fluoropyrrolidin-1-yl)propyl)-2-(3-(3-((4-methyl-4H-1,2,4-triazol-3-yl(methyl)oxetan-3-yl(phenyl)-4-(trifluoromethyl)isoindolin-1-one and 6-((R)-1-((S)-3-fluoropyrrolidin-1-yl)propyl)-2-(3-(3-((4-methyl-4H-1,2,4-triazol-3-yl)methyl)oxetan-3-yl)phenyl)-4-(trifluoromethyl)isoindolin-1-one

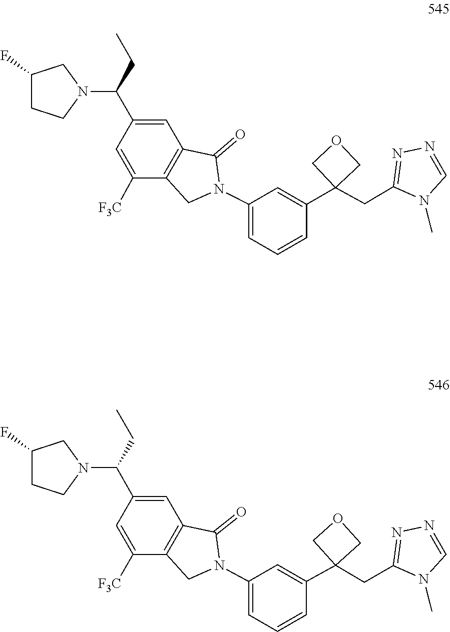 [SMILES: CC[C@@H](c1cc2c(c(C(F)(F)F)c1)CN(c1cccc(C3(Cc4nncn4C)COC3)c1)C2=O)N1CC[C@H](F)C1 ; CC[C@H](c1cc2c(c(C(F)(F)F)c1)CN(c1cccc(C3(Cc4nncn4C)COC3)c1)C2=O)N1CC[C@H](F)C1]

Step 1: Synthesis of 3-(methoxycarbonyl)-4-methyl-5-(trifluoromethyl)-benzoic acid: To a degassed solution of methyl 5-bromo-2-methyl-3-(trifluoromethyl)benzoate (18.0 g, 60.82 mmol), oxalic acid (11.5 g, 91.23 mmol), acetic anhydride (9.31 g, 91.23 mmol) and N-ethyl-N-isopropylpropan-2-amine (11.78 g, 91.23 mmol) in DMF (200 mL) were added Pd(OAc)₂ (1.36 g, 6.08 mmol) and XantPhos (1.76 g, 3.04 mmol). The mixture was stirred at 100° C. for 16 h under nitrogen. When the reaction was completed, the reaction was quenched by the addition of HCl (1 N) to pH 3. The aqueous solution was extracted with EtOAc (250 mL×3). The combined organic layer was washed with water and brine, dried and concentrated. The crude was purified by chromatography C to afford the title compound (7.5 g, 47%) as a light yellow solid.

Step 2: Synthesis of 4-(bromomethyl)-3-(methoxycarbonyl)-5-(trifluoromethyl)-benzoic acid: To a stirred solution of 4-methyl-3-(methoxycarbonyl)-5-(trifluoromethyl)benzoic acid (8 g, 30.5 mmol) and NBS (8.2 g, 46 mmol) in CCl₄ (160 mL) was added benzoyl peroxide (2.2 g, 9 mmol). The solution was stirred at 80° C. for 16 h. After concentration, the crude was purified by chromatography B to afford the title compound (8 g, 77%) as a yellow solid. MS (ESI) calculated for (C₁₁H₈BrF₃O₄) [M−H]339.0; found, 339.1. ¹H NMR (300 MHz, DMSO-d₆) δ 13.94 (s, 1H), 8.58 (d, J=1.8 Hz, 1H), 8.35 (d, J=1.8 Hz, 1H), 5.08 (s, 2H), 3.95 (s, 3H).

Step 3: Synthesis of 3-oxo-7-(trifluoromethyl)isoindoline-5-carboxylic acid. Ammonia (7M in MeOH, 2.5 mL) was added to 4-(bromomethyl)-3-(methoxycarbonyl)-5-(trifluoromethyl)benzoic acid (520 mg, 1.5 mmol) at ambient temperature. The reaction was stirred at the same temperature for 5 h. After removal of MeOH, 1N-HCl and water were added. The remaining solid was filtered and washed water. After drying under vacuum, the title compound was obtained in 380 mg (1.5 mmol, 100%) yield.

Step 4: Synthesis of N-methoxy-N-methyl-3-oxo-7-(trifluoromethyl)isoindoline-5-carboxamide. 3-Oxo-7-(trifluoromethyl)isoindoline-5-carboxylic acid (330 mg, 1.3 mmol), N,O-dimethylhydroxylamine hydrochloride (170 mg, 1.8 mmol), l-ethyl-3-(3-dimethylaminopropyl)carbodiimide hydrochloride (280 mg, 1.5 mmol) and hydroxybenzotriazole (14% wt water, 107 mg, 0.79 mmol) were dissolved in DMF (3 mL) at ambient temperature. Diisopropylethylamine (700 μL, 4.0 mmol) was added and the reaction was stirred at the same temperature overnight. The reaction was quenched by the addition of 1N-HCl and the product was extracted with DCM four times. The combined organic layer was dried and purified by silica gel column chromatography using methanol in EtOAc (0 to 40%) to afford the title compound in 246 mg (0.85 mmol, 64%) yield.

Step 5: Synthesis of 6-propionyl-4-(trifluoromethyl)isoindolin-1-one. A-methoxy-N-methyl-3-oxo-7-(trifluoromethyl)isoindoline-5-carboxamide (246 mg, 0.85 mmol) was dissolved in THF (4 mL). EtMgCl solution (2M in THF, 2.1 mL, 4.2 mmol) was added at 0° C. and the reaction was slowly warmed to ambient temperature. The reaction was quenched by the addition of sat. NH₄Cl solution. The layers were separated, and the aqueous layer was extracted twice more with EtOAc. The combined organic layer was dried and concentrated, the crude was purified by silica gel column chromatography using methanol in EtOAc (0 to 40%) to give the title compound in 166 mg (0.65 mmol, 76%) yield.

Step 6: Synthesis of 2-(3-(3-((4-methyl-4H-1,2,4-triazol-3-yl)methyl)oxetan-3-yl)phenyl)-6-propionyl-4-(trifluoromethyl)isoindolin-1-one. The N-arylation reaction was carried out in in a manner similar to 168, step 1 using 6-propionyl-4-(trifluoromethyl)isoindolin-1-one (166 mg, 0.65 mmol) and 3-((3-(3-bromophenyl)oxetan-3-yl)methyl)-4-methyl-4H-1,2,4-triazole (199 mg, 0.65 mmol) as reactants to afford the title compound in 158 mg (0.33 mmol, 50%) yield.

Step 7: Synthesis of 6-(l-((S)-3-fluoropyrrolidin-1-yl)propyl)-2-(3-(3-((4-methyl-4H-1,2,4-triazol-3-yl)methyl)oxetan-3-yl)phenyl)-4-(trifluoromethyl)isoindolin-1-one. Reductive amination of the methyl ketone was carried out according to the procedure of the procedure for 459, step 2 using 2-(3-(3-((4-methyl-4H-1,2,4-triazol-3-yl)methyl)oxetan-3-yl)phenyl)-6-propionyl-4-(trifluoromethyl)isoindolin-1-one (108 mg, 0.22 mmol) and (S)-3-fluoropyrrolidine hydrochloride (86 mg, 0.68 mmol) as reactants to afford the title compound in 58 mg (47%) yield.

Step 8: Synthesis of 6-((S)-1-((S)-3-fluoropyrrolidin-1-yl)propyl)-2-(3-(3-((4-methyl-4H-1,2,4-triazol-3-yl)methyl)oxetan-3-yl)phenyl)-4-(trifluoromethyl)isoindolin-1-one and 6-((R)-1-((S)-3-fluoropyrrolidin-1-yl)propyl)-2-(3-(3-((4-methyl-4H-1,2,4-triazol-3-yl)methyl)oxetan-3-yl)phenyl)-4-(trifluoromethyl)isoindolin-1-one. 6-(l-((S)-3-fluoro-pyrrolidin-1-yl)propyl)-2-(3-(3-((4-methyl-4H-1,2,4-triazol-3-yl)methyl)oxetan-3-yl)phenyl)-4-(trifluoromethyl)isoindolin-1-one (58 mg) was separated using column IG with CO₂ and methanol as mobile phase to afford 6-((S)-1-((S)-3-fluoropyrrolidin-1-yl)propyl)-2-(3-(3-((4-methyl-4H-1,2,4-triazol-3-yl)methyl)oxetan-3-yl)phenyl)-4-(trifluoromethyl)isoindolin-1-one (14 mg) and 6-((R)-1-((S)-3-fluoropyrrolidin-1-yl)propyl)-2-(3-(3-((4-methyl-4H-1,2,4-triazol-3-yl)methyl)oxetan-3-yl)phenyl)-4-(trifluoromethyl)isoindolin-1-one (18 mg).

¹H NMR (500 MHz, DMSO-d6) δ 8.20 (s, 1H), 7.97 (s, 1H), 7.94-7.85 (m, 2H), 7.41-7.33 (m, 2H), 6.78 (dt, J=7.6, 1.2 Hz, 1H), 5.29-5.09 (m, 3H), 4.98 (d, J=6.0 Hz, 2H), 4.90 (d, J=6.0 Hz, 2H), 3.52 (s, 2H), 3.48 (dt, J=9.3, 4.3 Hz, 1H), 2.90 (s, 3H), 2.19-2.01 (m, 1H), 1.90 (tdd, J=30.6, 13.4, 9.6 Hz, 2H), 1.71 (dt, J=14.5, 7.6 Hz, 1H), 0.65 (t, J=13 Hz, 3H); LCMS: C₂₉H₃₁F₄N₅O₂ requires: 557, found: m/z=558 [M+H]⁺.

¹H NMR (500 MHz, DMSO-d6) δ 8.20 (s, 1H), 7.97 (s, 1H), 7.94-7.85 (m, 2H), 7.41-7.33 (m, 2H), 6.78 (dt, J=7.6, 1.2 Hz, 1H), 5.29-5.09 (m, 3H), 4.98 (d, J=6.0 Hz, 2H), 4.90 (d, J=6.0 Hz, 2H), 3.52 (s, 2H), 3.48 (dt, J=9.3, 4.3 Hz, 1H), 2.90 (s, 3H), 2.19-2.01 (m, 1H), 1.90 (tdd, J=30.6, 13.4, 9.6 Hz, 2H), 1.71 (dt, J=14.5, 7.6 Hz, 1H), 0.65 (t, J=13 Hz, 3H); LCMS: C₂₉H₃₁F₄N₅O₂ requires: 557, found: m/z=558 [M+H]⁺.

### Example 547: 6-(1,1-difluoro-2-hydroxyethyl)-2-(3-(3-((4-methyl-4H-1,2,4-triazol-3-yl)methyl)oxetan-3-yl)phenyl)-4-(trifluoromethyl)isoindolin-1-one

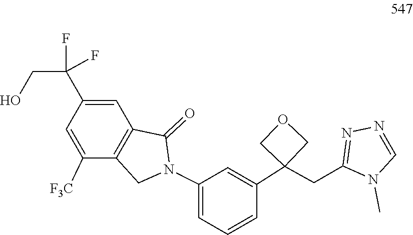 [SMILES: Cn1cnnc1CC1(c2cccc(N3Cc4c(cc(C(F)(F)CO)cc4C(F)(F)F)C3=O)c2)COC1]

Step 1: Synthesis of ethyl 2,2-difluoro-2-(2-(3-(3-((4-methyl-4H-1,2,4-triazol-3-yl)methyl)oxetan-3-yl)phenyl)-3-oxo-7-(trifluoromethyl)isoindolin-5-yl)acetate. The indolone formation reaction was carried out in a manner similar to 260, step 2 using Example R (165 mg, 0.67 mmol) and methyl 2-(bromomethyl)-5-(2-ethoxy-1,1-difluoro-2-oxoethyl)-3-(trifluoromethyl)benzoate (step-4 of Example 544) (283 mg, 0.68 mmol) as reactants to afford the title compound in 166 mg (45%) yield.

Step 2: Synthesis of 6-(1,1-difluoro-2-hydroxyethyl)-2-(3-(3-((4-methyl-4H-1,2,4-triazol-3-yl)methyl)oxetan-3-yl)phenyl)-4-(trifluoromethyl)isoindolin-1-one. Ethyl 2,2-difluoro-2-(2-(3-(3-((4-methyl-4H-1,2,4-triazol-3-yl)methyl)oxetan-3-yl)phenyl)-3-oxo-7-(trifluoromethyl)isoindolin-5-yl)acetate (166 mg, 0.30 mmol) was dissolved in MeOH (1 mL) at ambient temperature. NaBH₄ (25 mg, 0.67 mmol) was added and the reaction was stirred for 30 min. Another portion of NaBH₄ (18 mg, 0.48 mmol) was added and the reaction was stirred for another 45 min. The mixture was directly purified by chromatography B. The desired product was further purified using reverse phase HPLC to afford the title compound in 15 mg (9.5%) yield. ¹H NMR (500 MHz, DMSO-d6) δ 8.18 (s, 1H), 8.13 (s, 1H), 8.09 (s, 1H), 7.88 (ddd, J=8.2, 2.3, 1.0 Hz, 1H), 7.41 (t, J=1.9 Hz, 1H), 7.36 (t, J=7.9 Hz, 1H), 6.82-6.76 (m, 1H), 5.80-5.72 (m, 1H), 5.19 (s, 2H), 4.96 (d, J=6.0 Hz, 2H), 4.88 (d, J=6.1 Hz, 2H), 4.00 (td, J=13.4, 5.8 Hz, 2H), 3.51 (s, 2H), 2.91 (s, 3H); LCMS: C₂₄H₂₁F₅N₄O₃ requires: 508, found: m/z=509 [M+H]⁺.

### Example 548: 6-(((3R,4S)-4-fluoropyrrolidin-3-yl)(methyl)amino)-2-(3-((R)-1,1,2-trifluoro-1-(4-methyl-4H-1,2,4-triazol-3-yl)propan-2-yl)phenyl)-4-(trifluoro-methyl)isoindolin-1-one

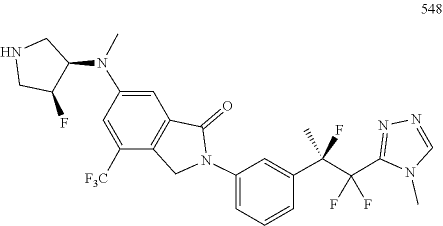 [SMILES: CN(c1cc2c(c(C(F)(F)F)c1)CN(c1cccc([C@@](C)(F)C(F)(F)c3nncn3C)c1)C2=O)[C@@H]1CNC[C@@H]1F]

Step 1: Synthesis of tert-butyl (3R,4S)-3-(((benzyloxy)carbonyl)amino)-4-fluoropyrrolidine-1-carboxylate: To a stirred solution of tert-butyl (3R,4S)-3-amino-4-fluoropyrrolidine-1-carboxylate (500 mg, 2.45 mmol) and DIEA (949 mg, 7.35 mmol) in DCM (10 mL) was added Cbz-Cl (833 mg, 4.90 mmol) at 0° C. The solution was stirred at 0-15° C. for 16 h. When the reaction was completed, the solvent was removed. The residue was purified by chromatography A to afford the title compound (670 mg, 81%) as a colorless oil. MS (ESI) calculated for (C₁₇H₂₃FN₂O₄) [M+H]⁺, 339.2; found, 339.0.

Step 2: Synthesis of tert-butyl (3R,4S)-3-(((benzyloxy)carbonyl)(methyl)amino)-4-fluoropyrrolidine-1-carboxylate: To a stirred solution of tert-butyl (3R,4S)-3-(((benzyloxy)carbonyl)amino)-4-fluoropyrrolidine-1-carboxylate (670 mg, 1.98 mmol) and Cs₂CO₃ (1.3 g, 3.96 mmol) in DMF (10 mL) was added Mel (562 mg, 3.96 mmol) at 0° C. The mixture was stirred at this temperature for 4 h. When the reaction was completed, the reaction was quenched by the addition of water. The aqueous solution was extracted with EtOAc. The combined organic solution was dried, filtered and concentrated. The residue was purified by chromatography A to afford the title compound (650 mg, 93%) as a colorless oil. MS (ESI) calculated for (C₁₈H₂₅FN₂O₄) [M+H]⁺, 353; found, 353.

Step 3: Synthesis of tert-butyl (3S,4R)-3-fluoro-4-(methylamino)pyrrolidine-1-carboxylate: To a solution of tert-butyl (3R,4S)-3-(((benzyloxy)carbonyl)(methyl)amino)-4-fluoropyrrolidine-1-carboxylate (650 mg, 1.85 mmol) in MeOH (15 mL) was added Pd/C (10%, 300 mg). The mixture was stirred at 20° C. for 16 h under hydrogen (2 atm). When the reaction was completed, the mixture was filtered. The filtrate was concentrated to give the residue which was purified by chromatography B to afford the title compound (320 mg, 79%) as a colorless oil. MS (ESI) calculated for (C₁₀H₁₉FN₂O₂) [M+H]⁺, 219; found, 219.

Step 4: Synthesis of tert-butyl (3S,4R)-3-fluoro-4-(methyl(3-oxo-2-(3-((R)-1,1,2-trifluoro-1-(4-methyl-4H-1,2,4-triazol-3-yl)propan-2-yl)phenyl)-7-(trifluoromethyl)iso-indolin-5-yl)amino)pyrrolidine-1-carboxylate. The C—N coupling reaction with potassium phosphate was carried out in a similar fashion as for 386 using (R)-6-Bromo-2-(3-(1,1,2-trifluoro-1-(4-methyl-4H-1,2,4-triazol-3-yl)propan-2-yl)phenyl)-4-(trifluoromethyl)-isoindolin-1-one (51 mg, 0.096 mmol) and tert-butyl (3S,4R)-3-fluoro-4-(methylamino)pyrrolidine-1-carboxylate (26 mg, 0.12 mmol) as reactants to afford the title compound in 18 mg (28%) yield.

Step 5: Synthesis of 6-(((3R,4S)-4-fluoropyrrolidin-3-yl)(methyl)amino)-2-(3-((R)-1,1,2-trifluoro-1-(4-methyl-4H-1,2,4-triazol-3-yl)propan-2-yl)phenyl)-4-(trifluoro-methyl)isoindolin-1-one. tert-butyl (3S,4R)-3-fluoro-4-(methyl(3-oxo-2-(3-((R)-1,1,2-trifluoro-1-(4-methyl-4H-1,2,4-triazol-3-yl)propan-2-yl)phenyl)-7-(trifluoromethyl)isoindolin-5-yl)amino)pyrrolidine-1-carboxylate (18 mg, 0.027 mmol) was dissolved in DCM (0.8 mL) at ambient temperature. TFA (200 μL) was added and the reaction was stirred for 2 h. Sat. NaHCO₃ solution was added and the desired product was extracted with DCM: MeOH (4:1) (×4). The combined organic layer was dried, concentrated and purified using reverse phase HPLC to give the title compound in 5.0 mg (32%) yield. ¹H NMR (500 MHz, DMSO-d6) δ 8.63 (s, 1H), 8.00 (d, J=2.0 Hz, 1H), 7.96-7.89 (m, 1H), 7.52 (t, J=8.0 Hz, 1H), 7.42-7.34 (m, 2H), 7.21 (d, J=7.5 Hz, 1H), 5.27 (dt, J=56.5, 4.6 Hz, 1H), 5.05 (s, 2H), 4.48-4.34 (m, 1H), 3.45 (s, 3H), 3.26-2.94 (m, 4H), 3.01 (s, 3H), 2.00 (d, J=24.3 Hz, 3H); LCMS: C₂₆H₂₅F₇N₆O requires: 570, found: m/z=571 [M+H]⁺.

### Example 549: 6-(((3S,4R)-4-fluoropyrrolidin-3-yl)(methyl)amino)-2-(3-((R)-1,1,2-trifluoro-1-(4-methyl-4H-1,2,4-triazol-3-yl)propan-2-yl)phenyl)-4-(trifluoromethyl)iso-indolin-1-one

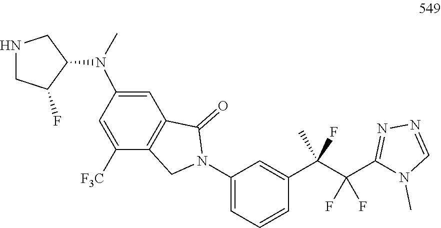 [SMILES: CN(c1cc2c(c(C(F)(F)F)c1)CN(c1cccc([C@@](C)(F)C(F)(F)c3nncn3C)c1)C2=O)[C@H]1CNC[C@H]1F]

Following Step 1 through Step 5 in the synthesis of 548 using tert-butyl (3S,4R)-3-amino-4-fluoropyrrolidine-1-carboxylate instead of tert-butyl (3R,4S)-3-amino-4-fluoro-pyrrolidine-1-carboxylate, the title compound was obtained in 2.5 mg yield. ¹H NMR (500 MHz, Methanol-d4) δ 8.50 (s, 1H), 7.95 (t, J=1.9 Hz, 1H), 7.83 (ddd, J=8.2, 2.3, 0.9 Hz, 1H), 7.53-7.46 (m, 2H), 7.38 (d, J=2.4 Hz, 1H), 7.24 (d, J=7.8 Hz, 1H), 5.28 (dt, J=56.2, 4.3 Hz, 1H), 5.01 (dd, J=3.5, 1.5 Hz, 2H), 4.39 (dtd, J=26.6, 8.5, 4.4 Hz, 1H), 3.12-3.23 (m, 2H), 3.09 (d, J=1.5 Hz, 3H), 2.02 (dt, J=24.1, 1.6 Hz, 3H); LCMS: C₂₆H₂₅F₇N₆O requires: 570, found: m/z=571 [M+H]⁺.

### Example 550: 6-(((3S,4S)-4-fluoropyrrolidin-3-yl)(methyl)amino)-2-(3-((R)-1,1,2-trifluoro-1-(4-methyl-4H-1,2,4-triazol-3-yl)propan-2-yl)phenyl)-4-(trifluoromethyl)iso-indolin-1-one

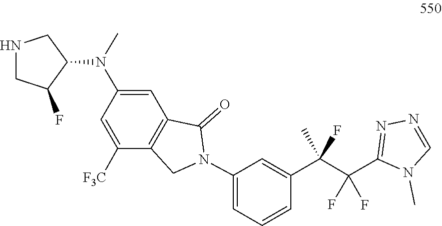 [SMILES: CN(c1cc2c(c(C(F)(F)F)c1)CN(c1cccc([C@@](C)(F)C(F)(F)c3nncn3C)c1)C2=O)[C@H]1CNC[C@@H]1F]

Following Step 1 through Step 5 in the synthesis of 548 using tert-butyl (3S,4S)-3-amino-4-fluoropyrrolidine-1-carboxylate instead of tert-butyl (3R,4S)-3-amino-4-fluoro-pyrrolidine-1-carboxylate, the title compound was obtained in 5.8 mg yield. ¹H NMR (500 MHz, DMSO-d6) δ 8.55 (s, 1H), 7.93 (t, J=1.9 Hz, 1H), 7.86-7.80 (m, 1H), 7.44 (t, J=8.0 Hz, 1H), 7.34-7.28 (m, 2H), 7.16-7.11 (m, 1H), 5.22-5.07 (m, 1H), 5.04-4.90 (m, 2H), 4.42 (dt, J=26.1, 7.0 Hz, 1H), 3.38 (s, 3H), 2.99 (dd, J=27.5, 4.0 Hz, 2H), 2.87 (s, 3H), 2.72 (dd, J=11.7, 6.5 Hz, 1H), 1.92 (d, J=24.3 Hz, 3H); LCMS: C₂₆H₂₅F₇N₆O requires: 570, found: m/z=571 [M+H]⁺.

### Example 551: 6-(((3R,4R)-4-fluoropyrrolidin-3-yl)(methyl)amino)-2-(3-((R)-1,1,2-trifluoro-1-(4-methyl-4H-1,2,4-triazol-3-yl)propan-2-yl)phenyl)-4-(trifluoromethyl)iso-indolin-1-one

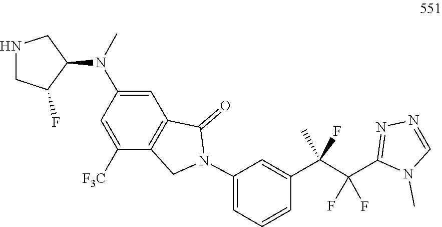 [SMILES: CN(c1cc2c(c(C(F)(F)F)c1)CN(c1cccc([C@@](C)(F)C(F)(F)c3nncn3C)c1)C2=O)[C@@H]1CNC[C@H]1F]

Following Step 1 through Step 5 in the synthesis of 548 using tert-butyl (3R,4R)-3-amino-4-fluoropyrrolidine-1-carboxylate instead of tert-butyl (3R,4S)-3-amino-4-fluoro-pyrrolidine-1-carboxylate, the title compound was obtained in 4.9 mg yield, ¹H NMR (500 MHz, DMSO-d6) δ 8.63 (s, 1H), 8.01 (s, 1H), 7.92 (dd, J=8.2, 2.2 Hz, 1H), 7.52 (t, J=8.0 Hz, 1H), 7.42-7.35 (m, 2H), 7.21 (d, J=7.8 Hz, 1H), 5.30-5.14 (m, 1H), 5.11-4.98 (m, 2H), 4.49 (d, J=26.4 Hz, 1H), 3.45 (s, 3H), 3.06 (dd, J=27.6, 3.9 Hz, 2H), 2.95 (s, 3H), 2.79 (dd, J=11.7, 6.4 Hz, 1H), 1.99 (d, J=24.2 Hz, 3H); LCMS: C₂₆H₂₅F₇N₆O requires: 570, found: m/z=571 [M+H]⁺.

### Example 552: 4-cyclopropyl-6-((4-fluoropiperidin-1-yl)methyl)-2-(3-(3-((4-methyl-4H-1,2,4-triazol-3-yl)methyl)oxetan-3-yl)phenyl)isoindolin-1-one

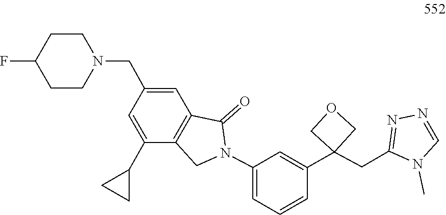 [SMILES: Cn1cnnc1CC1(c2cccc(N3Cc4c(cc(CN5CCC(F)CC5)cc4C4CC4)C3=O)c2)COC1]

Step 1: Synthesis of methyl 3-bromo-2-(bromomethyl)-5-iodobenzoate. Methyl 3-bromo-5-iodo-2-methylbenzoate (2.0 g, 5.6 mmol) was dissolved in CCl₄ (25 mL) at ambient temperature. NBS (1.2 g, 6.8 mmol) was added followed by benzoyl peroxide (26 mg, 0.11 mmol). The reaction was heated at 80° C. for two days. After cooling to ambient temperature, insoluble materials were filtered off and the filtrate was concentrated to afford the crude material which was used without purification.

Step 2: Synthesis of 4-bromo-6-iodo-2-(3-(3-((4-methyl-4H-1,2,4-triazol-3-yl)methyl)oxetan-3-yl)phenyl)isoindolin-1-one. The indolone formation reaction was carried out in a manner similar to 260, step 2 using Example R (1.24 g, 5.1 mmol) and crude methyl 3-bromo-2-(bromomethyl)-5-iodobenzoate (<5.6 mmol) as reactants to afford the title compound in 1.41 g (49%) yield.

Step 3: Synthesis of 4-bromo-2-(3-(3-((4-methyl-4H-1,2,4-triazol-3-yl)methyl)oxetan-3-yl)phenyl)-6-vinylisoindolin-1-one. 4-bromo-6-iodo-2-(3-(3-((4-methyl-4H-1,2,4-triazol-3-yl)methyl)oxetan-3-yl)phenyl)isoindolin-1-one (100 mg, 0.18 mmol), 2,4,6-trivinylboroxin-pyridine complex (14 mg, 0.056 mmol), tetrakis(triphenylphosphine)palladium(0) (8.9 mg, 0.0077 mmol) and K₂CO₃ (74 mg, 0.54 mmol) were added to a vial followed by DME (1 mL) and water (0.1 mL). The reaction mixture was purged with nitrogen and heated at 80° C. for 2 h. Upon cooling, the reaction mixture purifiec by chromatography to afford the title compound 57 mg (69%) yield.

Step 4: Synthesis of 4-cyclopropyl-2-(3-(3-((4-methyl-4H-1,2,4-triazol-3-yl)-methyl)oxetan-3-yl)phenyl)-6-vinylisoindolin-1-one. 4-bromo-2-(3-(3-((4-methyl-4H-1,2,4-triazol-3-yl)methyl)oxetan-3-yl)phenyl)-6-vinylisoindolin-1-one (57 mg, 0.12 mmol), 2-(2-dicyclohexylphosphanylphenyl)-N1,N1,N3,N3-tetramethyl-benzene-1,3-diamine (5.3 mg, 0.012 mmol), Pd(OAc)₂ (1.8 mg, 0.0081 mmol) were added to a vial. THF (0.5 mL) was added at ambient temperature followed by cyclopropylzinc bromide (0.5 M in THF, 0.61 mL, 0.31 mmol). The reaction was stirred at the same temperature for 2 h. More cyclopropylzinc bromide (0.5 M in THF, 0.61 mL, 0.31 mmol) was added and the reaction was allowed to stir overnight. Upon purification of product using chromatography B, the title compound was obtained in 41 mg (<79%) yield as an inseparable mixture with the starting material bromide.

Step 5: Synthesis of 7-cyclopropyl-2-(3-(3-((4-methyl-4H-1,2,4-triazol-3-yl)methyl)oxetan-3-yl)phenyl)-3-oxoisoindoline-5-carbaldehyde. The vinyl group was converted to the aldehyde according to the procedure described for 447, step 3 using the mixture of 4-cyclopropyl-2-(3-(3-((4-methyl-4H-1,2,4-triazol-3-yl)methyl)oxetan-3-yl)phenyl)-6-vinylisoindolin-1-one and 4-bromo-2-(3-(3-((4-methyl-4H-1,2,4-triazol-3-yl)methyl)oxetan-3-yl)phenyl)-6-vinylisoindolin-1-one (41 mg, <0.097 mmol) to give the title aldehyde in 25 mg (60%) yield as an inseparable mixture with 7-bromo-2-(3-(3-((4-methyl-4H-1,2,4-triazol-3-yl)methyl)oxetan-3-yl)phenyl)-3-oxoisoindoline-5-carbaldehyde.

Step 6: Synthesis of 4-cyclopropyl-6-((4-fluoropiperidin-1-yl)methyl)-2-(3-(3-((4-methyl-4H-1,2,4-triazol-3-yl(methyl)oxetan-3-yl)phenyl)isoindolin-1-one The reductive amination was carried out in a similar fashion as for 447, step 4 using a mixture of 7-cyclopropyl-2-(3-(3-((4-methyl-4H-1,2,4-triazol-3-yl)methyl)oxetan-3-yl)phenyl)-3-oxoisoindoline-5-carbaldehyde and 7-bromo-2-(3-(3-((4-methyl-4H-1,2,4-triazol-3-yl)methyl)oxetan-3-yl)phenyl)-3-oxoisoindoline-5-carbaldehyde (25 mg, 0.057 mmol) and 4-fluoropiperidine hydrochloride (20 mg, 0.14 mmol) as reactants to afford 4-cyclopropyl-6-((4-fluoropiperidin-1-yl)methyl)-2-(3-(3-((4-methyl-4H-1,2,4-triazol-3-yl)methyl)oxetan-3-yl)phenyl)isoindolin-1-one (5.8 mg).

¹H NMR (500 MHz, DMSO-d6) δ 8.21 (s, 1H), 7.95 (dd, J=8.3, 2.1 Hz, 1H), 7.49 (s, 1H), 7.38 (d, J=2.0 Hz, 1H), 7.35 (t, J=7.9 Hz, 1H), 7.11 (s, 1H), 6.74 (d, J=7.7 Hz, 1H), 5.00-4.94 (m, 4H), 4.91 (d, J=6.0 Hz, 2H), 4.69 (d, J=49.0 Hz, 1H), 3.54 (d, J=19.7 Hz, 4H), 2.90 (s, 3H), 2.30 (s, 1H), 2.03 (td, J=8.4, 4.2 Hz, 1H), 1.88-1.72 (m, 3H), 1.11-1.03 (m, 2H), 0.91-0.78 (m, 2H); LCMS: C₃₀H₃₄FN₅O₂ requires: 515, found: m/z=516 [M+H]⁺.

### Example 553a and 553b: (S)-6-(1-(4-fluoro-4-methylpiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one and (R)-6-(1-(4-fluoro-4-methylpiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one

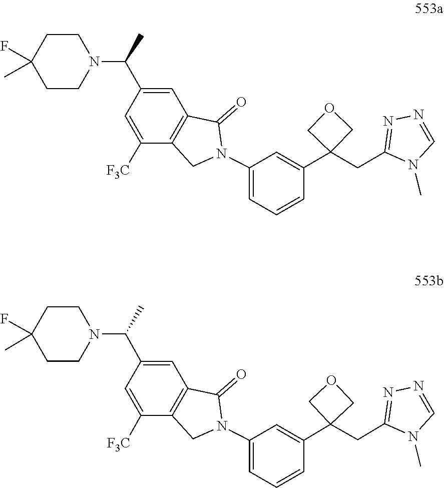 [SMILES: C[C@@H](c1cc2c(c(C(F)(F)F)c1)CN(c1cccc(C3(Cc4nncn4C)COC3)c1)C2=O)N1CCC(C)(F)CC1 ; C[C@H](c1cc2c(c(C(F)(F)F)c1)CN(c1cccc(C3(Cc4nncn4C)COC3)c1)C2=O)N1CCC(C)(F)CC1]

Step 1: Synthesis of l-(2-(3-(3-((4-methyl-4H-1,2,4-triazol-3-yl)methyl)oxetan-3-yl)phenyl)-3-oxo-7-(trifluoromethyl)isoindolin-5-yl)ethyl methanesulfonate. 6-(l-Hydroxyethyl)-2-(3-(3-((4-methyl-4H-1,2,4-triazol-3-yl)methyl)oxetan-3-yl)phenyl)-4-(trifluoromethyl)isoindolin-1-one (720 mg, 1.5 mmol) was suspended in DCM (12 mL) at 0° C. DMAP (370 mg, 3.0 mmol) was added followed by dropwise addition of methane sulfonic anhydride (320 mg, 1.9 mmol) in DCM (3 mL). The reaction was warmed to rt overnight. The reaction was quenched by addition of water and the product was extracted with DCM (×3). The combined organic layer was dried. Another batch of reaction was run using 170 mg (0.36 mmol) alcohol with diisopropylamine as the base. The reactions were combined and purified by chromatography B to afford the title compound in 784 mg (81%) yield.

Step 2: Synthesis of 6-(1-(4-fluoro-4-methylpiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one. 1-(2-(3-(3-((4-methyl-4H-1,2,4-triazol-3-yl)methyl)oxetan-3-yl)phenyl)-3-oxo-7-(trifluoromethyl)isoindolin-5-yl)ethyl methanesulfonate (390 mg, 0.71 mmol), K₂CO₃ (295 mg, 2.1 mmol) and 4-fluoro-4-methylpiperidine hydrochloride (215 mg, 1.4 mmol) were mixed together in DMF (1 mL) at ambient temperature overnight. Addition of water and extraction of products with DCM: MeOH (4:1) (×3), drying, followed by purification using chromatography B afforded 350 mg product which still contained unreacted mesylate. The mixture was re-subjected to reaction using K₂CO₃ (102 mg, 0.73 mmol) and 4-fluoro-4-methylpiperidine hydrochloride (57 mg, 0.37 mmol) in DMF (1.5 mL). The reaction was heated to 50° C. until complete conversion. Similar work up followed by similar purification afforded the title compound in 268 mg (66%) yield.

Step 3: Separation of (S)-6-(1-(4-fluoro-4-methylpiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one and (R)-6-(1-(4-fluoro-4-methylpiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one. 6-(1-(4-fluoro-4-methylpiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one (268 mg) was separated using column IC with CO₂ and mixture of methanol:acetonitrile (7:3) with 0.1% diethylamine as mobile phase to afford (S)-6-(1-(4-fluoro-4-methylpiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one (106 mg) and (R)-6-(1-(4-fluoro-4-methylpiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one (105 mg).

¹H NMR (500 MHz, DMSO-d6) δ 8.18 (s, 1H), 7.99 (s, 1H), 7.93 (s, 1H), 7.88 (ddd, J=8.2, 2.3, 0.9 Hz, 1H), 7.40-7.32 (m, 2H), 6.77 (dt, J=8.0, 1.2 Hz, 1H), 5.09 (s, 2H), 4.96 (d, J=6.0 Hz, 2H), 4.88 (d, J=6.1 Hz, 2H), 3.82 (q, J=6.7 Hz, 1H), 3.51 (s, 2H), 2.89 (s, 3H), 2.69 (d, J=11.1 Hz, 1H), 2.23 (q, J=6.9, 5.3 Hz, 2H), 1.81-1.53 (m, 4H), 1.37 (d, J=6.8 Hz, 3H), 1.29 (d, J=21.5 Hz, 3H); LCMS: C₃₀H₃₃F₄N₅O₂ requires: 571, found: m/z=572 [M+H]⁺.

¹H NMR (500 MHz, DMSO-d6) δ 8.18 (s, 1H), 7.99 (s, 1H), 7.93 (s, 1H), 7.88 (ddd, J=8.2, 2.2, 1.0 Hz, 1H), 7.39-7.32 (m, 2H), 6.77 (dt, J=8.1, 1.1 Hz, 1H), 5.13-5.05 (m, 2H), 4.96 (d, J=6.0 Hz, 2H), 4.88 (d, J=6.0 Hz, 2H), 3.82 (q, J=6.8 Hz, 1H), 3.51 (s, 2H), 2.89 (s, 3H), 2.69 (d, J=11.4 Hz, 1H), 2.28-2.16 (m, 2H), 1.81-1.54 (m, 4H), 1.37 (d, J=6.7 Hz, 3H), 1.29 (d, J=21.5 Hz, 3H); LCMS: C₃₀H₃₃F₄N₅O₂ requires: 571, found: m/z=572 [M+H]⁺.

### Example 554: (S)-4-cyclopropyl-6-((3-fluoropyrrolidin-1-yl)methyl)-2-(3-(3-((4-methyl-4H-1,2,4-triazol-3-yl)methyl)oxetan-3-yl)phenyl)isoindolin-1-one and (S)-4-bromo-6-((3-fluoropyrrolidin-1-yl)methyl)-2-(3-(3-((4-methyl-4H-1,2,4-triazol-3-yl)methyl)oxetan-3-yl)phenyl)isoindolin-1-one

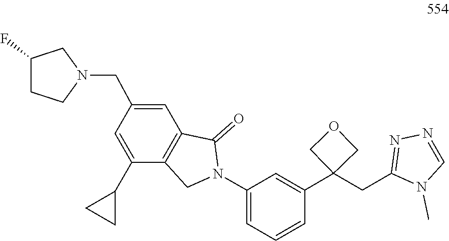 [SMILES: Cn1cnnc1CC1(c2cccc(N3Cc4c(cc(CN5CC[C@H](F)C5)cc4C4CC4)C3=O)c2)COC1]

The reductive amination was carried out in a similar fashion as for 447, step 4 using 7-cyclopropyl-2-(3-(3-((4-methyl-4H-1,2,4-triazol-3-yl)methyl)oxetan-3-yl)phenyl)-3-oxoisoindoline-5-carbaldehyde (41 mg, 0.095 mmol) and (S)-3-fluoropyrrolidine hydrochloride (38 mg, 0.30 mmol) as reactants to afford (S)-4-cyclopropyl-6-((3-fluoropyrrolidin-1-yl)methyl)-2-(3-(3-((4-methyl-4H-1,2,4-triazol-3-yl)methyl)oxetan-3-yl)phenyl)isoindolin-1-one (10 mg).

¹H NMR (500 MHz, Acetonitrile-d3) δ 7.96-7.91 (m, 1H), 7.89 (s, 1H), 7.51 (s, 1H), 7.37-7.27 (m, 2H), 7.13 (s, 1H), 6.72 (d, J=7.7 Hz, 1H), 5.16 (dt, J=55.9, 6.1 Hz, 1H), 5.02 (d, J=5.9 Hz, 2H), 4.97 (d, J=6.0 Hz, 2H), 4.82 (s, 2H), 3.67 (s, 2H), 3.52 (s, 2H), 2.90-2.74 (m, 2H), 2.87 (s, 3H), 2.60 (ddd, J=31.3, 11.6, 5.0 Hz, 1H), 2.34 (q, J=8.0 Hz, 1H), 2.30-2.08 (m, 1H), 2.05-1.80 (m, 1H), 1.13-1.00 (m, 2H), 0.79 (p, J=4.8, 4.2 Hz, 2H); LCMS: C₂₉H₃₂FN₅O₂ requires: 501, found: m/z=502 [M+H]⁺.

### Example 555: 6-((7-azabicyclo[2.2.1]heptan-7-yl)methyl)-2-(3-(3-((4-methyl-4H-1,2,4-triazol-3-yl)methyl)oxetan-3-yl)phenyl)-4-(trifluoromethyl)isoindolin-1-one

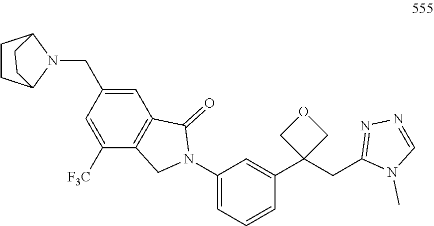 [SMILES: Cn1cnnc1CC1(c2cccc(N3Cc4c(cc(CN5C6CCC5CC6)cc4C(F)(F)F)C3=O)c2)COC1]

The reductive amination was carried out in a similar fashion as for 447, step 4 using Example Z (49 mg, 0.11 mmol) and 7-azabicyclo[2.2.1]heptane hydrochloride (29 mg, 0.22 mmol) as reactants to afford the title compound (14 mg, 23% yield) as a white solid, ¹H NMR (500 MHz, Methanol-d4) δ 8.19 (s, 1H), 8.10 (s, 1H), 8.04 (s, 1H), 7.80-7.74 (m, 1H), 7.44-7.37 (m, 2H), 6.78-6.72 (m, 1H), 5.14-5.02 (m, 6H), 3.80 (s, 2H), 3.66 (s, 2H), 2.90 (d, J=0.9 Hz, 3H), 1.89 (d, J=8.1 Hz, 5H), 1.44 (d, J=7.6 Hz, 5H); LCMS: C₂₉H₃₀F₃N₅O₂ requires: 537, found: m/z=538 [M+H]⁺.

### Example 556: 6-((8-azabicyclo[3.2.1]octan-8-yl)methyl)-2-(3-(3-((4-methyl-4H-1,2,4-triazol-3-yl)methyl)oxetan-3-yl)phenyl)-4-(trifluoromethyl)isoindolin-1-one

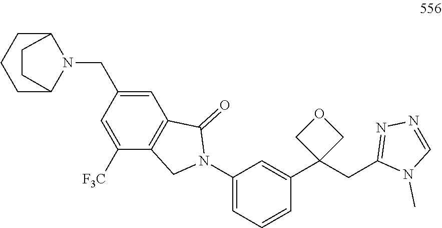 [SMILES: Cn1cnnc1CC1(c2cccc(N3Cc4c(cc(CN5C6CCCC5CC6)cc4C(F)(F)F)C3=O)c2)COC1]

The reductive amination was carried out in a similar fashion as for 447, step 4 using Example Z (50 mg, 0.11 mmol) and 8-azabicyclo[3.2.1]octane hydrochloride (33 mg, 0.22 mmol) as reactants to afford the title compound (36 mg, 60% yield) as a white solid. ¹H NMR (500 MHz, Methanol-d4) δ 8.19 (s, 1H), 8.11 (s, 1H), 8.05 (s, 1H), 7.80-7.74 (m, 1H), 7.43-7.37 (m, 2H), 6.75 (dt, J=7.5, 1.1 Hz, 1H), 5.13-5.03 (m, 6H), 3.75 (s, 2H), 3.66 (s, 2H), 3.19 (s, 2H), 2.90 (s, 3H), 2.13 (d, J=9.4 Hz, 2H), 1.79 (d, J=13.9 Hz, 2H), 1.68 (dq, J=34.1, 6.0 Hz, 3H), 1.55-1.46 (m, 1H), 1.41 (d, J=13.2 Hz, 2H); LCMS: C₃₀H₃₂F₃N₅O₂ requires: 551, found: m/z=552 [M+H]⁺.

### Example 557: 6-(((1R,3r,5S)-3-fluoro-8-azabicyclo[3.2.1]octan-8-yl)methyl)-2-(3-(3-((4-methyl-4H-1,2,4-triazol-3-yl)methyl)oxetan-3-yl)phenyl)-4-(trifluoromethyl)isoindolin-1-one

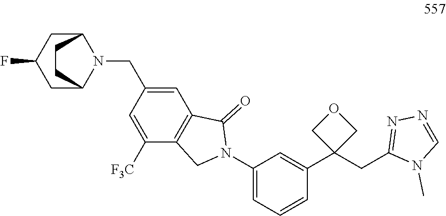 [SMILES: Cn1cnnc1CC1(c2cccc(N3Cc4c(cc(CN5[C@@H]6CC[C@H]5C[C@@H](F)C6)cc4C(F)(F)F)C3=O)c2)COC1]

The reductive amination was carried out in a similar fashion as for 447, step 4 using Example Z (50 mg, 0.11 mmol) and exo-3-fluoro-8-azabicyclo[3.2.1]octane hydrochloride (35 mg, 0.21 mmol) as reactants to afford the title compound (25 mg, 39% yield) as a white solid. ¹H NMR (500 MHz, Methanol-d4) δ 8.19 (s, 1H), 8.13 (s, 1H), 8.03 (s, 1H), 7.77 (ddd, J=8.2, 2.3, 0.9 Hz, 1H), 7.44-7.36 (m, 2H), 6.75 (dt, J=7.0, 1.0 Hz, 1H), 5.11-5.04 (m, 6H), 3.84 (s, 2H), 3.66 (s, 2H), 2.90 (s, 3H), 2.11 (dd, J=8.4, 4.2 Hz, 2H), 1.99 (ddd, J=10.0, 6.3, 2.9 Hz, 2H), 1.85 (q, J=13.8, 12.7 Hz, 2H), 1.73-1.63 (m, 2H); LCMS: C₃₀H₃₁F₄N₅O₂ requires: 569, found: m/z=570 [M+H]⁺.

### Example 558a and 558b: (S)-6-(1-(6-azaspiro[2.5]octan-6-yl)ethyl)-2-(3-(3-((4-methyl-4H-1,2,4-triazol-3-yl(methyl)oxetan-3-yl(phenyl)-4-(trifluoromethyl)isoindolin-1-one and (R)-6-(1-(6-azaspiro[2.5]octan-6-yl)ethyl)-2-(3-(3-((4-methyl-4H-1,2,4-triazol-3-yl)methyl)-oxetan-3-yl)phenyl)-4-(trifluoromethyl)isoindolin-1-one

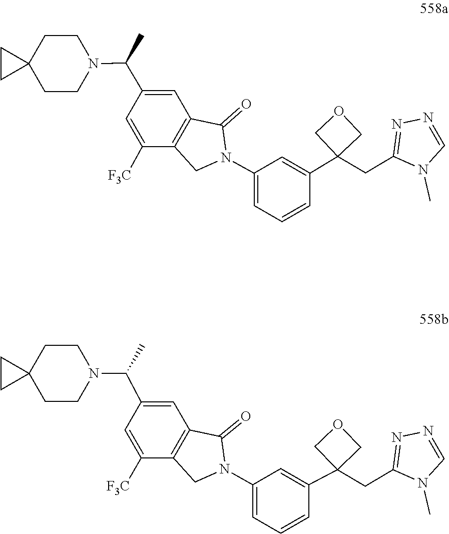 [SMILES: C[C@@H](c1cc2c(c(C(F)(F)F)c1)CN(c1cccc(C3(Cc4nncn4C)COC3)c1)C2=O)N1CCC2(CC1)CC2 ; C[C@H](c1cc2c(c(C(F)(F)F)c1)CN(c1cccc(C3(Cc4nncn4C)COC3)c1)C2=O)N1CCC2(CC1)CC2]

Step 1: Synthesis of 6-(1-(6-azaspiro[2.5]octan-6-yl)ethyl)-2-(3-(3-((4-methyl-4H-1,2,4-triazol-3-yl)methyl)oxetan-3-yl)phenyl)-4-(trifluoromethyl)isoindolin-1-one. Reductive amination of the methyl ketone was carried out according to the procedure for 459, step 2 using 6-acetyl-2-(3-(3-((4-methyl-4H-1,2,4-triazol-3-yl)methyl)oxetan-3-yl)phenyl)-4-(trifluoromethyl)isoindolin-1-one (52 mg, 0.11 mmol) and 6-azaspiro[2.5]octane hydrochloride (46 mg, 0.31 mmol) as reactants to afford the title compound in 32 mg (52%) yield.

Step 2: Separation of (S)-6-(1-(6-azaspiro[2.5]octan-6-yl)ethyl)-2-(3-(3-((4-methyl-4H-1,2,4-triazol-3-yl)methyl)oxetan-3-yl)phenyl)-4-(trifluoromethyl)isoindolin-1-one and (R)-6-(1-(6-azaspiro[2.5]octan-6-yl)ethyl)-2-(3-(3-((4-methyl-4H-1,2,4-triazol-3-yl)methyl)oxetan-3-yl)phenyl)-4-(trifluoromethyl)isoindolin-1-one. 6-(1-(6-azaspiro 2.5 octan-6-yl)ethyl)-2-(3-(3-((4-methyl-4H-1,2,4-triazol-3-yl)methyl)oxetan-3-yl)phenyl)-4-(trifluoromethyl)isoindolin-1-one (30 mg) was separated using column (R, R) Whelk-0 with CO₂ and 25% methanol with 0.1% ammonium hydroxide as mobile phase to afford (S)-6-(1-(6-azaspiro[2.5]octan-6-yl)ethyl)-2-(3-(3-((4-methyl-4H-1,2,4-triazol-3-yl)methyl)oxetan-3-yl)phenyl)-4-(trifluoromethyl)isoindolin-1-one (3 mg) and (R)-6-(1-(6-azaspiro 2.5 octan-6-yl)ethyl)-2-(3-(3-((4-methyl-4H-1,2,4-triazol-3-yl)methyl)oxetan-3-yl)phenyl)-4-(trifluoromethyl)isoindolin-1-one (2 mg).

¹H NMR (500 MHz, Acetonitrile-d3) δ 8.01 (s, 1H), 7.95 (s, 1H), 7.93-7.88 (m, 2H), 7.37 (t, J=8.0 Hz, 1H), 7.33 (t, J=2.0 Hz, 1H), 6.78 (dt, J=7.8, 1.2 Hz, 1H), 5.04 (d, J=6.1 Hz, 2H), 5.01-4.94 (m, 4H), 3.77 (q, J=6.8 Hz, 1H), 3.54 (s, 2H), 2.88 (s, 3H), 2.59-2.37 (m, 4H), 1.42 (d, J=6.8 Hz, 3H), 1.40-1.30 (m, 4H), 0.25 (s, 4H); LCMS: C₃₁H₃₄F₃N₅O₂ requires: 565, found: m/z=566 [M+H]⁺.

¹H NMR (500 MHz, Acetonitrile-d3) δ 8.01 (s, 1H), 7.95 (s, 1H), 7.93-7.88 (m, 2H), 7.37 (t, J=8.0 Hz, 1H), 7.33 (t, J=2.0 Hz, 1H), 6.78 (dt, J=7.8, 1.2 Hz, 1H), 5.04 (d, J=6.1 Hz, 2H), 5.01-4.92 (m, 4H), 3.77 (q, J=6.8 Hz, 1H), 3.54 (s, 2H), 2.88 (s, 3H), 2.58-2.36 (m, 4H), 1.42 (d, J=6.8 Hz, 3H), 1.40-1.30 (m, 4H), 0.25 (s, 4H); LCMS: C₃₁H₃₄F₃N₅O₂ requires: 565, found: m/z=566 [M+H]⁺.

### Example 559: 6-(l-azabicyclo[2.2.2]oct-2-en-3-yl)-2-(3-(3-((4-methyl-4H-1,2,4-triazol-3-yl)methyl)oxetan-3-yl)phenyl)-4-(trifluoromethyl)isoindolin-1-one

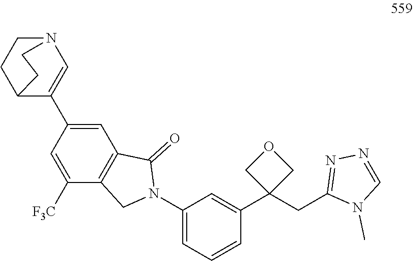 [SMILES: Cn1cnnc1CC1(c2cccc(N3Cc4c(cc(C5=CN6CCC5CC6)cc4C(F)(F)F)C3=O)c2)COC1]

A dioxane (2 mL) solution of l-azabicyclo[2.2.2]oct-2-en-3-yl trifluoromethane-sulfonate (102 mg, 0340 mmol) (PCT Int. Appl., 2017188287) was added to a capped vial, followed by addition of KOAc (84 mg, 0.86 mmol), 4,4,4′,4′,5,5,5′,5′-octamethyl-2,2′-bi(1,3,2-dioxaborolane) (38 mg, 0.15 mmol), dppf (7.3 mg, 0.013 mmol) and PdCl₂dppf (21 mg, 0.029 mmol). Vial was purged with N₂, sealed and heated to 95° C. for 4 h. More 4,4,4′,4′,5,5,5′,5′-octamethyl-2,2′-bi(1,3,2-dioxaborolane) (97 mg, 0.38 mmol), PdCl₂dppf (16 mg, 0.021 mmol) and KOAc (not weighed) were added and further heated at 95° C. for 2 h. Upon cooling, K₂CO₃ (86 mg, 0.62 mmol), 6-bromo-2-(3-(3-((4-methyl-4H-1,2,4-triazol-3-yl)methyl)oxetan-3-yl)phenyl)-4-(trifluoromethyl)isoindolin-1-one (194 mg, 0.38 mmol), PdCl₂dppf (16 mg, 0.022 mmol) and water (60 μL) were added. After purging with N₂, the sealed vial was heated at 95° C. overnight. After cooling, the mixture was diluted with water and DCM then filtered. The product was extracted using DCM: MeOH (4:1) (×2). After drying and concentrating, the product was purified by chromatography B to afford the title compound in 43 mg (21%) yield. Further HPLC purification gave the analytically pure sample. ¹H NMR (500 MHz, Acetonitrile-d3) δ 8.07 (s, 1H), 7.99 (s, 1H), 7.91-7.90 (m, 2H), 7.37 (t, J=8.0 Hz, 1H), 7.34 (t, J=2.0 Hz, 1H), 7.01 (d, J=1.8 Hz, 1H), 6.79 (d, J=7.5 Hz, 1H), 5.04 (d, J=6.1 Hz, 2H), 5.02-4.95 (m, 4H), 3.54 (s, 2H), 3.27 (s, 1H), 2.98 (ddd, J=13.4, 9.1, 4.7 Hz, 2H), 2.88 (s, 3H), 2.56 (td, J=11.2, 4.8 Hz, 2H), 1.84 (m, 2H), 1.60 (m, 2H); LCMS: C₂₉H₂₈F₃N₅O₂ requires: 535, found: m/z=536 [M+H]⁺.

### Example 560: 2-(3-(3-((4-methyl-4H-1,2,4-triazol-3-yl)methyl)oxetan-3-yl)phenyl)-6-(quinuclidin-3-yl)-4-(trifluoromethyl)isoindolin-1-one

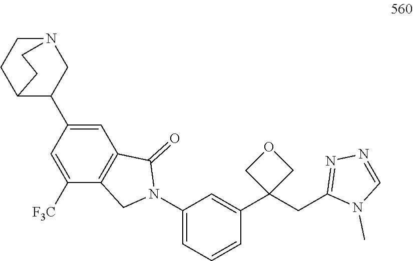 [SMILES: Cn1cnnc1CC1(c2cccc(N3Cc4c(cc(C5CN6CCC5CC6)cc4C(F)(F)F)C3=O)c2)COC1]

6-(1-Azabicyclol[2.2.2]oct-2-en-3-yl)-2-(3-(3-((4-methyl-4H-1,2,4-triazol-3-yl)methyl)oxetan-3-yl)phenyl)-4-(trifluoromethyl)isoindolin-1-one (21 mg, 0.039 mmol) was dissolved in MeOH (1 mL). Pd/C (10% wet, 6.0 mg) was added and hydrogenated over balloon overnight. After filtering off Pd/C and concentration, the residue was purified by HPLC to afford the title compound in 19 mg (90%) yield. 1H NMR (500 MHz, Acetonitrile-d3) δ 7.99 (d, J=1.5 Hz, 1H), 7.94-7.89 (m, 2H), 7.85 (s, 1H), 7.41-7.31 (m, 2H), 6.78 (dt, J=7.8, 1.2 Hz, 1H), 5.04 (d, J=6.1 Hz, 2H), 5.02-4.92 (m, 4H), 3.54 (s, 2H), 3.36 (ddd, J=13.2, 10.1, 2.4 Hz, 1H), 3.21 (t, J=8.7 Hz, 1H), 3.13-3.03 (m, 1H), 3.02-2.74 (m, 5H), 1.86-1.68 (m, 1H), 1.64-1.51 (m, 1H), 1.42 (ddd, J=13.6, 10.0, 4.9 Hz, 1H); LCMS: C₂₉H₃₀F₃N₅O₂ requires: 537, found: m/z=538 [M+H]⁺.

### Example 561: 3-(2-(3-(3-((4-Methyl-4H-1,2,4-triazol-3-yl)methyl)oxetan-3-yl)phenyl)-3-oxo-7-(trifluoromethyl)isoindolin-5-yl)oxazolidin-2-one

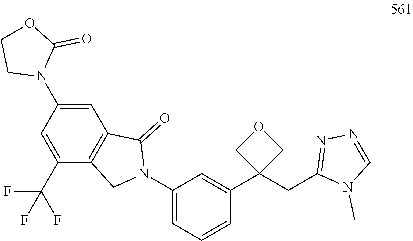 [SMILES: Cn1cnnc1CC1(c2cccc(N3Cc4c(cc(N5CCOC5=O)cc4C(F)(F)F)C3=O)c2)COC1]

The C—N coupling reaction with potassium phosphate was carried out in a similar fashion as for Example 386 using 6-bromo-2-(3-{3-[(4-methyl-4H-1,2,4-triazol-3-yl)methyl]-oxetan-3-yl}phenyl)-4-(trifluoromethyl)-2,3-dihydro-1H-isoindol-1-one (50 mg, 0.099 mmol, 1 eq.) and 1,3-oxazolidin-2-one (10 mg, 0.12 mmol, 1.2 eq.) as reactants afforded the title compound (28 mg, 55%) as a pink solid: ¹H NMR (500 MHz, DMSO-d₆) δ 8.20 (d, J=2.0 Hz, 1H), 8.12 (s, 1H), 8.05 (d, J=2.1 Hz, 1H), 7.86-7.80 (m, 1H), 7.35-7.25 (m, 2H), 6.71 (dt, J=7.7, 1.3 Hz, 1H), 5.08-4.97 (m, 2H), 4.90 (d, J=6.0 Hz, 2H), 4.82 (d, J=6.2 Hz, 2H), 4.48-4.41 (m, 2H), 4.17 (dd, J=9.0, 6.9 Hz, 2H), 3.45 (s, 2H), 2.84 (s, 3H); LCMS: C₂₅H₂₂F₃N₅O₄ requires: 513, found: m/z=514 [M+H]⁺.

### Example 562: 6-((1R,4R)-5-Methyl-2,5-diazabicyclo[2.2.1]heptan-2-yl)-2-(3-((R)-1,1,2-trifluoro-1-(4-methyl-4H-1,2,4-triazol-3-yl)propan-2-yl)phenyl)-4-(trifluoromethyl)-isoindolin-1-one

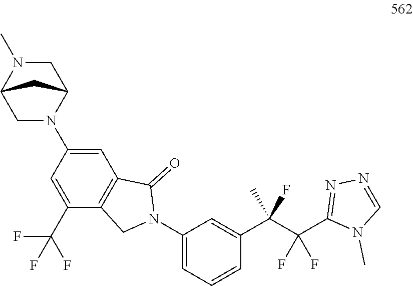 [SMILES: CN1C[C@H]2C[C@@H]1CN2c1cc2c(c(C(F)(F)F)c1)CN(c1cccc([C@@](C)(F)C(F)(F)c3nncn3C)c1)C2=O]

The C—N coupling reaction with potassium phosphate was carried out in a similar fashion as for Example 386 using 6-bromo-2-{3-[(2R)-1,1,2-trifluoro-1-(4-methyl-4H-1,2,4-triazol-3-yl)propan-2-yl]phenyl}-4-(trifluoromethyl)-2,3-dihydro-1H-isoindol-1-one (50 mg, 0.094 mmol, 1 eq.) and (1R,4R)-2-methyl-2,5-diazabicyclo[2.2.1]heptane dihydrobromide (31 mg, 0.11 mmol, 1.2 eq.) as reactants afforded the title compound (22 mg, 41%) as an off-white solid: ¹H NMR (500 MHz, DMSO-d₆) δ 8.63 (s, 1H), 7.98 (d, J=2.0 Hz, 1H), 7.91 (dd, J=8.1, 2.1 Hz, 1H), 7.51 (t, J=8.1 Hz, 1H), 7.21 (d, J=8.1 Hz, 1H), 7.15-7.09 (m, 2H), 5.08-4.97 (m, 2H), 4.56 (s, 1H), 3.47 (d, J=10.0 Hz, 1H), 3.44 (s, 3H), 3.26 (d, J=9.0 Hz, 1H), 2.85 (dd, J=9.6, 2.0 Hz, 1H), 2.45 (d, J=9.6 Hz, 2H), 2.27 (s, 3H), 2.00 (d, J=24.3 Hz, 3H), 1.92 (d, J=9.4 Hz, 1H), 1.81 (d, J=9.4 Hz, 1H); LCMS: C₂₇H₂₆F₆N₆O requires: 564, found: m/z=565 [M+H]⁺.

### Example 563: 6-((1S,4S)-5-Methyl-2,5-diazabicyclo[2.2.1]heptan-2-yl)-2-(3-((R)-1,1,2-trifluoro-1-(4-methyl-4H-1,2,4-triazol-3-yl)propan-2-yl)phenyl)-4-(trifluoromethyl)-isoindolin-1-one

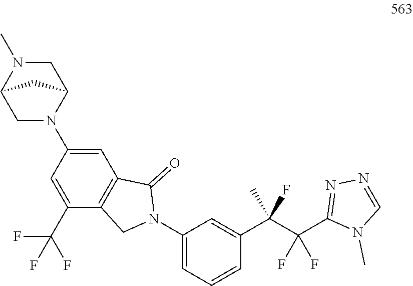

The C—N coupling reaction with potassium phosphate was carried out in a similar fashion as for Example 386 using 6-bromo-2-{3-[(2R)-1,1,2-trifluoro-1-(4-methyl-4H-1,2,4-triazol-3-yl)propan-2-yl]phenyl}-4-(trifluoromethyl)-2,3-dihydro-1H-isoindol-1-one (50 mg, 0.094 mmol, 1 eq.) and (1S,4S)-2-methyl-2,5-diazabicyclo[2.2.1]heptane dihydrobromide (31 mg, 0.11 mmol, 1.2 eq.) as reactants afforded the title compound (25 mg, 46%) as an off-white solid: 1H NMR (500 MHz, DMSO-d₆) δ 8.63 (s, 1H), 7.99 (d, J=2.1 Hz, 1H), 7.91 (dd, J=8.1, 2.1 Hz, 1H), 7.51 (t, J=8.0 Hz, 1H), 7.21 (d, J=7.9 Hz, 1H), 7.15-7.09 (m, 2H), 5.08-4.96 (m, 2H), 4.56 (s, 1H), 3.47 (d, J=10.3 Hz, 1H), 3.44 (s, 3H), 3.26 (d, J=9.1 Hz, 1H), 2.85 (dd, J=9.6, 2.0 Hz, 1H), 2.45 (d, J=9.5 Hz, 2H), 2.27 (s, 3H), 2.00 (d, J=24.2 Hz, 3H), 1.92 (d, J=9.5 Hz, 1H), 1.84-1.79 (m, 1H); LCMS: C₂₇H₂₆F₆N₆O requires: 564, found: m/z=565 [M+H]⁺.

### Example 564: 6-(8-Methyl-3,8-diazabicyclo[3.2.1]octan-3-yl)-2-(3-((R)-1,1,2-trifluoro-1-(4-methyl-4H-1,2,4-triazol-3-yl)propan-2-yl)phenyl)-4-(trifluoromethyl)isoindolin-1-one

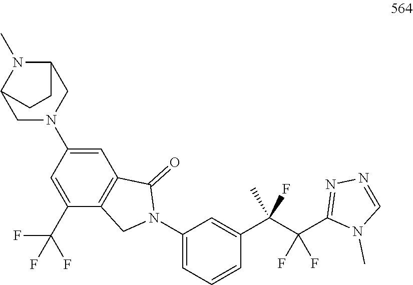 [SMILES: CN1C2CCC1CN(c1cc3c(c(C(F)(F)F)c1)CN(c1cccc([C@@](C)(F)C(F)(F)c4nncn4C)c1)C3=O)C2]

The C—N coupling reaction with potassium phosphate was carried out in a similar fashion as for Example 386 using 6-bromo-2-{3-[(2R)-1,1,2-trifluoro-1-(4-methyl-4H-1,2,4-triazol-3-yl)propan-2-yl]phenyl}-4-(trifluoromethyl)-2,3-dihydro-1H-isoindol-1-one (50 mg, 0.094 mmol, 1 eq.) and (1R,5S)-8-methyl-3,8-diazabicyclo[3.2.1]octane dihydrobromide (31 mg, 0.11 mmol, 1.2 eq.) as reactants afforded the title compound (25 mg, 46%) as an off-white solid: ¹H NMR (500 MHz, DMSO-d₆) δ 8.63 (s, 1H), 8.00 (d, J=2.1 Hz, 1H), 7.94-7.91 (m, 1H), 7.51 (t, J=8.0 Hz, 1H), 7.34 (dd, J=15.3, 2.3 Hz, 2H), 7.23-7.19 (m, 1H), 5.10-4.99 (m, 2H), 3.55 (dd, J=10.9, 2.3 Hz, 2H), 3.46 (s, 3H), 3.29-3.21 (m, 2H), 2.96 (dd, J=10.6, 2.3 Hz, 2H), 2.25 (s, 3H), 2.04-1.91 (m, 5H), 1.66 (q, J=6.2 Hz, 2H); LCMS: C₂₈H₂₈F₆N₆O requires: 578, found: m/z=579 [M+H]⁺.

### Example 565: 6-(5-Methyl-2,5-diazabicyclo[2.2.2]octan-2-yl)-2-(3-((R)-1,1,2-trifluoro-1-(4-methyl-4H-1,2,4-triazol-3-yl)propan-2-yl)phenyl)-4-(trifluoromethyl)isoindolin-1-one

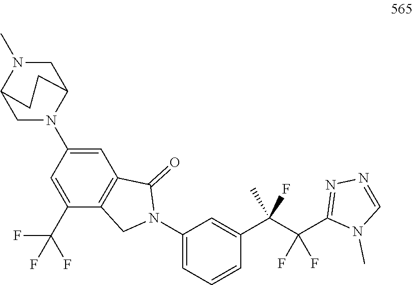 [SMILES: CN1CC2CCC1CN2c1cc2c(c(C(F)(F)F)c1)CN(c1cccc([C@@](C)(F)C(F)(F)c3nncn3C)c1)C2=O]

The C—N coupling reaction with potassium phosphate was carried out in a similar fashion as for Example 386 using 6-bromo-2-{3-[(2R)-1,1,2-trifluoro-1-(4-methyl-4H-1,2,4-triazol-3-yl)propan-2-yl]phenyl}-4-(trifluoromethyl)-2,3-dihydro-1H-isoindol-1-one (50 mg, 0.094 mmol, 1 eq.) and 2-methyl-2,5-diazabicyclo[2.2.2]octane dihydrochloride (22 mg, 0.11 mmol, 1.2 eq.) as reactants afforded the title compound (15 mg, 27%) as an off-white solid: ¹H NMR (500 MHz, DMSO-d₆) δ 8.63 (s, 1H), 8.00 (s, 1H), 7.92 (dd, J=8.2, 2.2 Hz, 1H), 7.51 (t, J=8.0 Hz, 1H), 7.20 (d, J=15.9 Hz, 3H), 5.08-4.96 (m, 2H), 4.12 (s, 1H), 3.73 (dd, J=10.2, 2.6 Hz, 1H), 3.45 (s, 3H), 3.25 (dd, J=10.1, 2.1 Hz, 1H), 2.91 (dd, J=10.3, 2.2 Hz, 1H), 2.87-2.77 (m, 2H), 2.34 (s, 3H), 2.00 (d, J=24.3 Hz, 3H), 1.90-1.70 (m, 3H), 1.62-1.54 (m, 1H); LCMS: C₂₈H₂₈F₆N₆O requires: 578, found: m/z=579 [M+H]⁺.

### Example 566: 6-(3-Methyl-3,6-diazabicyclo[3.1.1]heptan-6-yl)-2-(3-((R)-1,1,2-trifluoro-1-(4-methyl-4H-1,2,4-triazol-3-yl)propan-2-yl)phenyl)-4-(trifluoromethyl)isoindolin-1-one

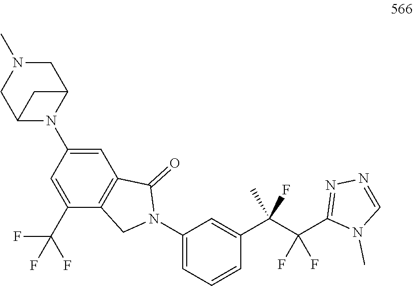 [SMILES: CN1CC2CC(C1)N2c1cc2c(c(C(F)(F)F)c1)CN(c1cccc([C@@](C)(F)C(F)(F)c3nncn3C)c1)C2=O]

The C—N coupling reaction with potassium phosphate was carried out in a similar fashion as for Example 386 using 6-bromo-2-{3-[(2R)-1,1,2-trifluoro-1-(4-methyl-4H-1,2,4-triazol-3-yl)propan-2-yl]phenyl}-4-(trifluoromethyl)-2,3-dihydro-1H-isoindol-1-one (50 mg, 0.094 mmol, 1 eq.) and 3-methyl-3,6-diazabicyclo[3.1.1]heptane (13 mg, 0.11 mmol, 1.2 eq.) as reactants afforded the title compound (5.2 mg, 10%) as an off-white solid: ¹H NMR (500 MHz, DMSO-d₆) δ 8.63 (d, J=3.3 Hz, 1H), 8.03-7.93 (m, 1H), 7.93-7.85 (m, 1H), 7.51 (td, J=8.0, 4.3 Hz, 1H), 7.24-7.13 (m, 2H), 7.11-7.05 (m, 1H), 5.08-4.96 (m, 2H), 4.49 (d, J=5.8 Hz, 1H), 3.95-3.80 (m, 1H), 3.43 (d, J=9.9 Hz, 3H), 2.98 (d, J=11.2 Hz, 1H), 2.76 (d, J=11.1 Hz, 1H), 2.32 (s, 1H), 2.20 (s, 1H), 2.12 (s, 3H), 2.04-1.93 (m, 3H), 1.92 (d, J=7.5 Hz, 1H), 1.71 (dd, J=13.0, 7.0 Hz, 1H); LCMS: C₂₇H₂₆F₆N₆O requires: 564, found: m/z=565 [M+H]⁺.

### Example 567: 2-(3-(3-((4-Methyl-4H-1,2,4-triazol-3-yl)methyl)oxetan-3-yl)phenyl)-6-morpholino-4-(trifluoromethyl)isoindolin-1-one

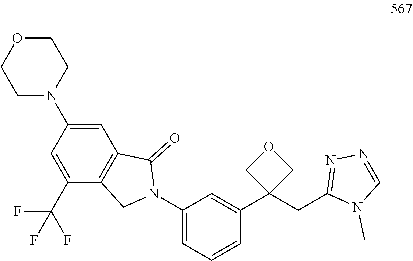 [SMILES: Cn1cnnc1CC1(c2cccc(N3Cc4c(cc(N5CCOCC5)cc4C(F)(F)F)C3=O)c2)COC1]

The C—N coupling reaction with potassium phosphate was carried out in a similar fashion as for Example 386 using Example Y (50 mg, 0.094 mmol, 1 eq.) and morpholine (10 μL, 0.11 mmol, 1.2 eq.) as reactants afforded the title compound (21 mg, 42%) as an off-white solid: ¹H NMR (500 MHz, DMSO-d₆) δ 8.20 (s, 1H), 7.90 (ddd, J=8.2, 2.2, 0.9 Hz, 1H), 7.54 (d, J=2.3 Hz, 1H), 7.48 (d, J=2.3 Hz, 1H), 7.39-7.32 (m, 2H), 6.76 (dt, J=7.9, 1.2 Hz, 1H), 5.13-4.94 (m, 4H), 4.90 (d, J=6.1 Hz, 2H), 3.78 (dd, J=6.1, 3.6 Hz, 4H), 3.52 (s, 2H), 3.29 (s, 4H), 2.91 (s, 3H); LCMS: C₂₆H₂₆F₃N₅O₃ requires: 513, found: m/z=514 [M+H]⁺.

### Example 568: 4,6-Dicyclopropyl-2-(3-(3-((4-methyl-4H-1,2,4-triazol-3-yl)methyl)oxetan-3-yl)phenyl)-2,3-dihydro-1H-pyrrolo[3,4-c]pyridin-1-one

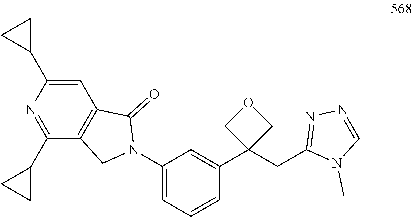 [SMILES: Cn1cnnc1CC1(c2cccc(N3Cc4c(cc(C5CC5)nc4C4CC4)C3=O)c2)COC1]

Step 1: Synthesis of 4,6-dicyclopropyl-2,3-dihydro-1H-pyrrolo[3,4-c]pyridin-1-one A solution of 4,6-dichloro-1H,2H,3H-pyrrolo[3,4-c]pyridin-1-one (500 mg, 2.5 mmol, 1 eq.), palladium acetate (55 mg, 0.25 mmol, 0.1 eq.) and 2′-(dicyclohexylphosphanyl)-N2,N2,N6,N6-tetramethyl-[1,1′-biphenyl]-2,6-diamine (220 mg, 0.50 mmol, 0.2 eq.) in THF (6.0 mL) was treated with a solution of bromo(cyclopropyl)zinc in THF (0.5 M, 15 mL, 7.4 mmol, 3 eq.) at rt. The mixture was stirred at rt for 2 hours, quenched with sat ammonium chloride and extracted with EtOAc (3×). The combined organic layers were washed with brine (lx), dried (Na₂SO₄), filtered and concentrated. Purification on SiO₂ (ethanol in EtOAc) followed by recrystallization from DCM afforded the title compound as a white solid.

Step 2: Synthesis of 4,6-dicyclopropyl-2-(3-(3-((4-methyl-4H-1,2,4-triazol-3-yl)methyl)oxetan-3-yl)phenyl)-2,3-dihydro-1H-pyrrolo[3,4-c]pyridin-1-one. The C—N coupling reaction with cesium carbonate was carried out in a similar fashion as for Example 386 using Example N (100 mg, 0.32 mmol, 1 eq.) and 4,6-dicyclopropyl-2,3-dihydro-1H-pyrrolo[3,4-c]pyridin-1-one (83 mg, 0.39 mmol, 1.2 eq.) as reactants afforded the title compound (52 mg, 36%) as an off-white solid: ¹H NMR (500 MHz, DMSO-d₆) δ 8.21 (s, 1H), 7.96 (dd, J=8.3, 2.1 Hz, 1H), 7.42 (t, J=1.9 Hz, 1H), 7.40-7.33 (m, 2H), 6.78 (dt, J=7.8, 1.2 Hz, 1H), 5.05 (s, 2H), 4.97 (d, J=6.0 Hz, 2H), 4.90 (d, J=6.0 Hz, 2H), 3.52 (s, 2H), 2.93 (s, 3H), 2.21 (tt, J=7.8, 4.8 Hz, 1H), 2.15 (tt, J=7.7, 4.9 Hz, 1H), 1.11-1.00 (m, 4H), 0.99-0.89 (m, 4H); LCMS: C₂₆H₂₇N₅O₂ requires: 441, found: m/z=442 [M+H]⁺.

### Example 569: 2-(3-(3-((4-Methyl-4H-1,2,4-triazol-3-yl)methyl)oxetan-3-yl)phenyl)-6-((1R,4R)-5-methyl-2,5-diazabicyclo[2.2.1]heptan-2-yl)-4-(trifluoromethyl)isoindolin-1-one

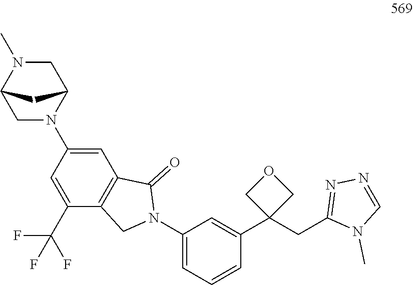

The C—N coupling reaction with potassium phosphate was carried out in a similar fashion as for Example 386 using Example Y (50 mg, 0.094 mmol, 1 eq.) and (1R,4R)-2-methyl-2,5-diazabicyclo[2.2.1]heptane dihydrobromide (32 mg, 0.12 mmol, 1.2 eq.) as reactants afforded the title compound (13 mg, 25%) as an off-white solid: ¹H NMR (500 MHz, DMSO-d₆) δ 8.19 (s, 1H), 7.92-7.86 (m, 1H), 7.38-7.31 (m, 2H), 7.10 (t, J=2.4 Hz, 2H), 6.75 (d, J=7.6 Hz, 1H), 5.08-4.84 (m, 6H), 4.55 (s, 1H), 3.52 (s, 2H), 3.50-3.41 (m, 2H), 3.25 (d, J=9.0 Hz, 1H), 2.90 (s, 3H), 2.84 (dd, J=9.6, 2.1 Hz, 1H), 2.45 (d, J=9.3 Hz, 1H), 2.27 (s, 3H), 1.92 (d, J=9.6 Hz, 1H), 1.81 (d, J=9.2 Hz, 1H); LCMS: C₂₈H₂₉F₃N₆O₂ requires: 538, found: m/z=539 [M+H]⁺.

### Example 570 and 571: 2-(3-(3-(Difluoro(4-methyl-4H-1,2,4-triazol-3-yl)methyl)oxetan-3-yl)phenyl)-6-((S)-1-((S)-3-fluoropyrrolidin-1-yl)ethyl)-4-(trifluoromethyl)isoindolin-1-one and 2-(3-(3-(difluoro(4-methyl-4H-1,2,4-triazol-3-yl)methyl)oxetan-3-yl)phenyl)-6-((R)-1-((S)-3-fluoropyrrolidin-1-yl)ethyl)-4-(trifluoromethyl)isoindolin-1-one

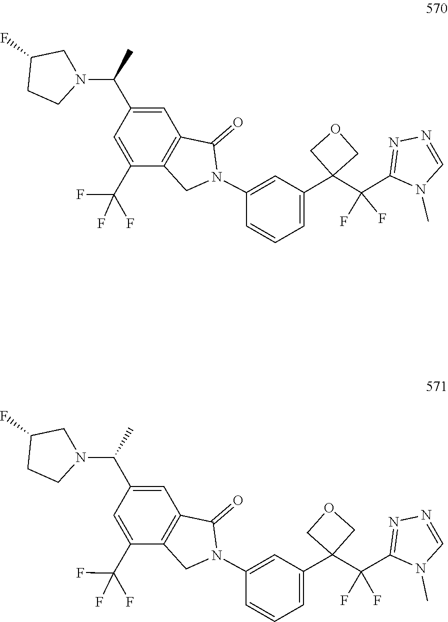 [SMILES: C[C@@H](c1cc2c(c(C(F)(F)F)c1)CN(c1cccc(C3(C(F)(F)c4nncn4C)COC3)c1)C2=O)N1CC[C@H](F)C1 ; C[C@H](c1cc2c(c(C(F)(F)F)c1)CN(c1cccc(C3(C(F)(F)c4nncn4C)COC3)c1)C2=O)N1CC[C@H](F)C1]

Step 1: Synthesis of 2-(3-(3-(difluoro(4-methyl-4H-1,2,4-triazol-3-yl)methyl)-oxetan-3-yl)phenyl)-6-(l-((S)-3-fluoropyrrolidin-1-yl)ethyl)-4-(trifluoromethyl)isoindolin-1-one. The C—N coupling reaction with potassium phosphate was carried out in a similar fashion as for Example 386 using 3-{[3-(3-bromophenyl)oxetan-3-yl]difluoromethyl}-4-methyl-1,2,4-triazole (145 mg, 0.42 mmol, 1 eq.) and 6-{l-[(3S)-3-fluoropyrrolidin-1-yl]ethyl}-4-(trifluoromethyl)-2,3-dihydroisoindol-1-one (147 mg, 0.46 mmol, 1.1 eq.) as reactants afforded the title compound (134 mg, 55%) as an off-white solid.

Step 2: The enantiomers (0.150 g) were separated using chiral chromatography on a CHIRALPAK OZ column with CO₂ and methanol as mobile phase to afford 2-(3-(3-(difluoro(4-methyl-4H-1,2,4-triazol-3-yl)methyl)oxetan-3-yl)phenyl)-6-((S)-1-((S)-3-fluoropyrrolidin-1-yl)ethyl)-4-(trifluoromethyl)isoindolin-1-one (82 mg) and 2-(3-(3-(difluoro(4-methyl-4H-1,2,4-triazol-3-yl)methyl)oxetan-3-yl)phenyl)-6-((R)-1-((S)-3-fluoropyrrolidin-1-yl)ethyl)-4-(trifluoromethyl)isoindolin-1-one (68 mg) as off-white solids.

2-(3-(3-(difluoro(4-methyl-4H-1,2,4-triazol-3-yl)methyl)oxetan-3-yl)phenyl)-6-((S)-1-((S)-3-fluoropyrrolidin-1-yl)ethyl)-4-(trifluoromethyl)isoindolin-1-one: ¹H NMR (500 MHz, DMSO-d₆) δ 8.48 (s, 1H), 8.00 (d, J=27.5 Hz, 2H), 7.93 (dd, J=8.1, 2.1 Hz, 1H), 7.52 (t, J=2.0 Hz, 1H), 7.44 (t, J=8.0 Hz, 1H), 6.95 (d, J=7.7 Hz, 1H), 5.33 (d, J=6.9 Hz, 2H), 5.30-5.14 (m, 1H), 5.13-5.08 (m, 2H), 5.05 (d, J=6.8 Hz, 2H), 3.64 (q, J=6.6 Hz, 1H), 3.11 (s, 3H), 2.90 (dd, J=26.9, 11.5 Hz, 1H), 2.70-2.55 (m, 2H), 2.41 (q, J=7.9 Hz, 1H), 2.15 (ddd, J=22.6, 14.6, 7.3 Hz, 1H), 1.95-1.85 (m, 1H), 1.37 (d, J=6.6 Hz, 3H); LCMS: C₂₈H₂₇F₅N₅O₂ requires: 579, found: m/z=580 [M+H]⁺.

(S)-2-(3-(1-(4-methylisoxazol-3-yl)propan-2-yl)phenyl)-4-(trifluoromethyl)isoindolin-1-one: 1H NMR (500 MHz, DMSO-d₆) δ 8.48 (s, 1H), 8.02 (s, 1H), 7.97 (s, 1H), 7.95-7.91 (m, 1H), 7.53 (t, J=2.0 Hz, 1H), 7.44 (t, J=8.0 Hz, 1H), 6.95 (d, J=8.0 Hz, 1H), 5.33 (d, J=7.0 Hz, 2H), 5.26-5.12 (m, 1H), 5.12-5.08 (m, 2H), 5.05 (d, J=6.8 Hz, 2H), 3.62 (q, J=6.6 Hz, 1H), 3.11 (s, 3H), 2.94 (td, J=8.2, 5.0 Hz, 1H), 2.68-2.56 (m, 2H), 2.31-2.22 (m, 1H), 2.17 (dp, J=20.2, 7.2, 6.7 Hz, 1H), 1.92 (ddt, J=29.7, 14.5, 7.0 Hz, 1H), 1.38 (d, J=6.6 Hz, 3H); LCMS: C₂₈H₂₇F₆N₅O₂ requires: 579, found: m/z=580 [M+H]⁺.

### Example 572: 2-Cyclopropyl-N-(3-(3-((4-methyl-4H-1,2,4-triazol-3-yl)methyl)oxetan-3-yl)phenyl)-6-(3-methyl-3,6-diazabicyclo[3.1.1]heptan-6-yl)pyrimidine-4-carboxamide

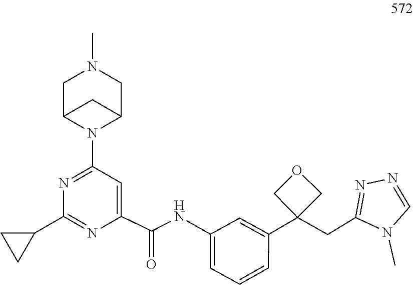 [SMILES: CN1CC2CC(C1)N2c1cc(C(=O)Nc2cccc(C3(Cc4nncn4C)COC3)c2)nc(C2CC2)n1]

Step 1: Synthesis of methyl 2-cyclopropyl-6-(3-methyl-3,6-diazabicyclo[3.1.1]-heptan-6-yl)pyrimidine-4-carboxylate The C—N coupling reaction with potassium phosphate was carried out in a similar fashion as for Example 386 using ethyl 6-chloro-2-cyclopropyl-pyrimidine-4-carboxylate (50 mg, 0.22 mmol, 1 eq.) and 3-methyl-3,6-diazabicyclo[3.1.1]-heptane (30 mg, 0.26 mmol, 1.2 eq.) as reactants afforded the title compound (33 mg, 53%) as an off-white solid.

Step 2: Synthesis of 2-cyclopropyl-6-(3-methyl-3,6-diazabicyclo[3.1.1]heptan-6-yl)pyrimidine-4-carboxylic acid A solution of methyl 2-cyclopropyl-6-(3-methyl-3,6-diazabicyclo[3.1.1]heptan-6-yl)pyrimidine-4-carboxylate (33 mg, 0.12 mmol, 1 eq.) in THF (0.41 mL) was treated with an aqueous solution of lithium hydroxide (1M, 0.17 mL, 0.17 mmol, 1.5 eq.) at rt. The mixture was stirred for 72 h at rt and quenched with hydrochloric acid (2M, 0.085 mL, 0.17 mmol, 1.5 eq.). THF was removed and the mixture was dried on a lyophilizer to yield the title compound.

Step 3: 2-cyclopropyl-N-(3-(3-((4-methyl-4H-1,2,4-triazol-3-yl)methyl)oxetan-3-yl)phenyl)-6-(3-methyl-3,6-diazabicyclo[3.1.1]heptan-6-yl)pyrimidine-4-carboxamide. The amide bond formation reaction was carried out in a similar fashion as for Example 56 using the 2-cyclopropyl-6-(3-methyl-3,6-diazabicyclo[3.1.1]heptan-6-yl)pyrimidine-4-carboxylic acid (0.12 mmol, 1 eq.) and Example R (29 mg, 0.16 mmol, 1 eq.) as reactants afforded the title compound (6.8 mg, 11%) as an off-white solid: ¹H NMR (500 MHz, DMSO-d₆) δ 10.24 (s, 1H), 8.21 (s, 1H), 7.85-7.72 (m, 1H), 7.45 (d, J=2A Hz, 1H), 7.28 (t, J=8.0 Hz, 1H), 6.86 (s, 1H), 6.67 (dt, J=7.8, 1.3 Hz, 1H), 4.94 (d, J=6.2 Hz, 2H), 4.86 (d, J=6.4 Hz, 2H), 4.60-4.40 (m, 2H), 3.49 (s, 2H), 2.92 (s, 3H), 2.88-2.83 (m, 1H), 2.18 (s, 3H), 2.14-2.06 (m, 1H), 1.91 (d, J=7.9 Hz, 1H), 1.55-1.40 (m, 4H), 0.95-0.85 (m, 4H); LCMS: C₂₇H₃₂N₈O₂ requires: 500, found: m/z=501 [M+H]⁺.

### Example 573: 2-Cyclopropyl-N-(3-(3-((4-methyl-4H-1,2,4-triazol-3-yl)methyl)oxetan-3-yl)phenyl)-6-((1R,5S)-3-methyl-3,8-diazabicyclo[3.2.1]octan-8-yl)pyrimidine-4-carboxamide

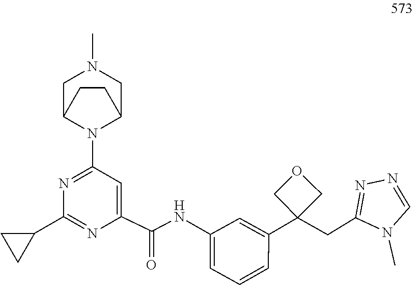 [SMILES: CN1CC2CCC(C1)N2c1cc(C(=O)Nc2cccc(C3(Cc4nncn4C)COC3)c2)nc(C2CC2)n1]

Step 1: Synthesis of methyl 2-cyclopropyl-6-((1R,5S)-3-methyl-3,8-diazabicyclo-[3.2.1]octan-8-yl)pyrimidine-4-carboxylate The C—N coupling reaction with potassium phosphate was carried out in a similar fashion as for Example 386 using ethyl 6-chloro-2-cyclopropylpyrimidine-4-carboxylate (50 mg, 0.22 mmol, 1 eq.) and 3-methyl-3,8-diazabicyclo-[3.2.1]octane dihydrochloride (53 mg, 0.26 mmol, 1.2 eq.) as reactants afforded the title compound (27 mg, 39%) as an off-white solid.

Step 2: Synthesis of 2-cyclopropyl-6-((1R,5S)-3-methyl-3,8-diazabicyclo[3.2.1]octan-8-yl)pyrimidine-4-carboxylic acid A solution of methyl 2-cyclopropyl-6-((1R,5S)-3-methyl-3,8-diazabicyclo[3.2.1]octan-8-yl)pyrimidine-4-carboxylate (37 mg, 0.086 mmol, 1 eq.) in THF (0.31 mL) was treated with an aqueous solution of lithium hydroxide (1M, 0.13 mL, 0.13 mmol, 1.5 eq.) at rt. The mixture was stirred for 72 h at rt and quenched with hydrochloric acid (2M, 0.065 mL, 0.13 mmol, 1.5 eq.). THF was removed, and the mixture was dried on a lyophilizer to yield the title compound.

Step 3: Synthesis of 2-cyclopropyl-N-(3-(3-((4-methyl-4H-1,2,4-triazol-3-yl)methyl)oxetan-3-yl)phenyl)-6-((1R,5S)-3-methyl-3,8-diazabicyclo[3.2.1]octan-8-yl)pyrimidine-4-carboxamide. The amide bond formation reaction was carried out in a similar fashion as for Example 56 using the 2-cyclopropyl-6-((1R,5S)-3-methyl-3,8-diazabicyclo[3.2.1]octan-8-yl)pyrimidine-4-carboxylic acid (0.086 mmol, 1 eq.) and Example R (21 mg, 0.086 mmol, 1 eq.) as reactants afforded the title compound (27 mg, 60%) as an off-white solid: ¹H NMR (500 MHz, DMSO-d₆) δ 10.15 (s, 1H), 8.13 (s, 1H), 7.71 (d, J=8.2 Hz, 1H), 7.37 (t, J=1.9 Hz, 1H), 7.20 (t, J=7.9 Hz, 1H), 6.99 (s, 1H), 6.63-6.57 (m, 1H), 4.86 (d, J=6.0 Hz, 2H), 4.79 (d, J=6.0 Hz, 2H), 3.41 (s, 2H), 2.85 (s, 3H), 2.60-2.54 (m, 2H), 2.48-2.44 (m, 4H), 2.10-1.99 (m, 4H), 1.81 (d, J=39.0 Hz, 4H), 0.99 (s, 2H), 0.90 (dd, J=7.9, 4.3 Hz, 2H); LCMS: C₂₈H₃₄N₈O₂ requires: 514, found: m/z=515 [M+H]⁺.

### Example 574: (S)-2-Cyclopropyl-6-((3-fluoropyrrolidin-1-yl)methyl)-N-(3-(3-((4-methyl-4H-1,2,4-triazol-3-yl)methyl)oxetan-3-yl)phenyl)pyrimidine-4-carboxamide

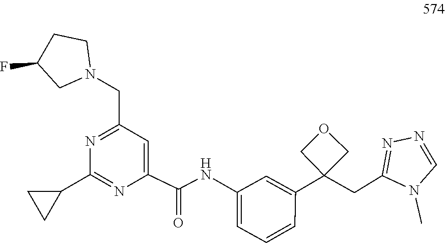 [SMILES: Cn1cnnc1CC1(c2cccc(NC(=O)c3cc(CN4CC[C@H](F)C4)nc(C4CC4)n3)c2)COC1]

Step 1: Synthesis of (S)-2-cyclopropyl-6-((3-fluoropyrrolidin-1-yl)methyl)-pyrimidine-4-carboxylic acid A solution of ethyl (S)-2-cyclopropyl-6-((3-fluoropyrrolidin-1-yl)methyl)pyrimidine-4-carboxylate (200 mg, 0.68 mmol, 1 eq.) in THF (2.4 mL) was treated with an aqueous solution of lithium hydroxide (1M, 1.0 mL, 1.0 mmol, 1.5 eq.) at rt. The mixture was stirred for 2 h at rt and quenched with hydrochloric acid (2M, 0.5 mL, 1.0 mmol, 1.5 eq.). THF was removed and the mixture was dried on a lyophilizer to yield the title compound.

Step 2: Synthesis of (S)-2-cyclopropyl-6-((3-fluoropyrrolidin-1-yl)methyl)-N-(3-(3-((4-methyl-4H-1,2,4-triazol-3-yl)methyl)oxetan-3-yl)phenyl)pyrimidine-4-carboxamide. The amide bond formation reaction was carried out in a similar fashion as for Example 56 using the (S)-2-cyclopropyl-6-((3-fluoropyrrolidin-1-yl)methyl)pyrimidine-4-carboxylic acid (0.68 mmol, 1 eq.) and Example R (0.18 g, 0.68 mmol, 1 eq.) as reactants afforded the title compound (44 mg, 13%) as an off-white solid: ¹H NMR (500 MHz, DMSO-d₆) δ 10.41 (s, 1H), 8.19 (s, 1H), 7.83 (s, 1H), 7.81-7.74 (m, 1H), 7.47 (q, J=2.3 Hz, 1H), 7.28 (t, J=7.9 Hz, 1H), 6.68 (dt, J=7.7, 1.3 Hz, 1H), 5.30-5.15 (m, 1H), 4.93 (d, J=6.0 Hz, 2H), 4.85 (d, J=6.0 Hz, 2H), 3.79 (d, J=1.8 Hz, 2H), 3.48 (s, 3H), 2.92 (s, 2H), 2.92-2.82 (m, 2H), 2.72 (ddd, J=31.9, 11.6, 4.9 Hz, 1H), 2.47-2.38 (m, 1H), 2.25-2.09 (m, 1H), 2.37-2.30 (m, 1H), 2.00-1.84 (m, 1H), 1.20-1.07 (m, 4H); LCMS: C₂₆H₃₀FN₇O₂ requires: 491, found: m/z=492 [M+H]⁺.

### Example 575 and 576: 6-((R)-1-(5-Azaspiro[2.4]heptan-5-yl)ethyl)-2-(3-(3-((S)-fluoro(4-methyl-4H-1,2,4-triazol-3-yl)methyl)oxetan-3-yl)phenyl)-4-(trifluoromethyl)isoindolin-1-one and 6-((R)-1-(5-azaspiro[2.4]heptan-5-yl)ethyl)-2-(3-(3-((R)-fluoro(4-methyl-4H-1,2,4-triazol-3-yl)methyl)oxetan-3-yl)phenyl)-4-(trifluoromethyl)isoindolin-1-one

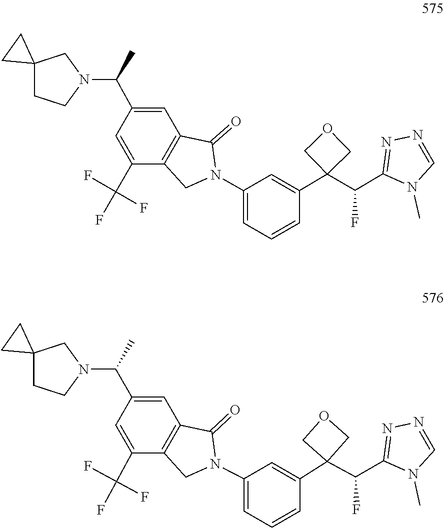 [SMILES: C[C@@H](c1cc2c(c(C(F)(F)F)c1)CN(c1cccc(C3([C@@H](F)c4nncn4C)COC3)c1)C2=O)N1CCC2(CC2)C1 ; C[C@H](c1cc2c(c(C(F)(F)F)c1)CN(c1cccc(C3([C@@H](F)c4nncn4C)COC3)c1)C2=O)N1CCC2(CC2)C1]

Step 1: The enantiomers of 3-{[3-(3-bromophenyl)oxetan-3-yl](fluoro)methyl}-4-methyl-1,2,4-triazole (2.0 g) were separated using chiral chromatography on a CHIRALPAK IC column with CO₂ and methanol as mobile phase to afford 3-[(R)-[3-(3-bromophenyl)oxetan-3-yl](fluoro)methyl]-4-methyl-1,2,4-triazole (830 mg, shorter retention time)) and 3-[(S)-[3-(3-bromophenyl)oxetan-3-yl](fluoro)methyl]-4-methyl-1,2,4-triazole (844 mg, longer retention time) as off-white solids.

Step 2: Synthesis of 6-acetyl-4-(trifluoromethyl)-2,3-dihydroisoindol-1-one A solution of ammonia in methanol (7 M, 59 mmol, 10 eq.) was mixed with methyl 5-acetyl-2-(bromomethyl)-3-(trifluoromethyl)benzoate (2.0 g, 5.9 mmol, 1 eq.) and stirred at rt for 3h. The solvent was evaporated to give the product.

Step 3: Synthesis of 6-acetyl-2-(3-{3-[(R)-fluoro(4-methyl-1,2,4-triazol-3-yl)methyl]oxetan-3-yl}phenyl)-4-(trifluoromethyl)-3H-isoindol-1-one. The C—N coupling reaction with cesium carbonate was carried out in a similar fashion as for Example 386 using 3-[(R)-[3-(3-bromophenyl)oxetan-3-yl](fluoro)methyl]-4-methyl-1,2,4-triazole (780 mg, 2.39 mmol, 1 eq.) and 6-acetyl-4-(trifluoromethyl)-2,3-dihydroisoindol-1-one (698 mg, 2.9 mmol, 1.2 eq.) as reactants afforded the title compound (348 mg, 30%) as a brown solid.

Step 4: Synthesis of 6-(l-{5-azaspiro[2.4]heptan-5-yl}ethyl)-2-(3-{3-[(R)-fluoro(4-methyl-1,2,4-triazol-3-yl)methyl]oxetan-3-yl}phenyl)-4-(trifluoromethyl)-3H-isoindol-1-one. The reductive amination reaction with sodium cyanoborohydride was carried out in a similar fashion as for Example 459 using 3-[(R)-[3-(3-bromophenyl)oxetan-3-yl](fluoro)methyl]-4-methyl-1,2,4-triazole (780 mg, 2.39 mmol, 1 eq.) and 6-acetyl-4-(trifluoromethyl)-2,3-dihydroisoindol-1-one (698 mg, 2.9 mmol, 1.2 eq.) as reactants afforded the title compound (348 mg, 30%) as a brown solid.

Step 5: The enantiomers (0.25 g) were separated using chiral chromatography on a CHIRALPAK IE column with CO₂ and methanol as mobile phase to afford 6-[(1S)-1-{5-azaspiro[2,4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one (75 mg, shorter retention time) and 6-[(1R)-1-{5-azaspiro[2.4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one (41 mg, longer retention time) as off-white solids.

6-[(1S)-1-{5-azaspiro[2.4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one: ¹H NMR (500 MHz, DMSO-d₆) δ 8.36 (s, 1H), 8.02 (s, 1H), 7.99-7.91 (m, 2H), 7.56 (d, J=2.0 Hz, 1H), 7.39 (t, J=8.0 Hz, 1H), 6.99 (d, J=7.6 Hz, 1H), 6.28 (d, J=45 Hz, 1H), 5.38 (d, J=6.7 Hz, 1H), 5.23 (d, J=6.2 Hz, 1H), 5.18-5.04 (m, 3H), 4.84 (dd, J=6.2, 3.9 Hz, 1H), 3.56 (q, J=6.6 Hz, 1H), 3.19 (s, 3H), 2.75 (q, J=7.7 Hz, 1H), 2.60-2.40 (m, 2H), 2.33 (d, J=8.8 Hz, 1H), 1.75 (qt, J=12.3, 6.7 Hz, 2H), 1.35 (d, J=6.5 Hz, 3H), 0.59-0.40 (m, 4H); LCMS: C₃₀H₃₁F₄N₅O₂ requires: 569, found: m/z=570 [M+H]⁺.

6-[(1R)-1-{5-azaspiro[2.4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one: ¹H NMR (500 MHz, DMSO-d₆) δ 8.36 (s, 1H), 8.02 (s, 1H), 7.99-7.91 (m, 2H), 7.56 (t, J=2.0 Hz, 1H), 7.39 (t, J=8.0 Hz, 1H), 6.99 (d, J=7.6 Hz, 1H), 6.29 (d, J=45 Hz, 1H), 5.38 (d, J=6.7 Hz, 1H), 5.23 (d, J=6.4 Hz, 1H), 5.17-5.04 (m, 3H), 4.84 (dd, J=6.2, 3.9 Hz, 1H), 3.56 (q, J=6.5 Hz, 1H), 3.19 (s, 3H), 2.79-2.72 (m, 1H), 2.60-2.40 (m, 2H), 2.33 (d, J=8.8 Hz, 1H), 1.75 (ddq, J=19.4, 12.5, 6.8 Hz, 2H), 1.35 (d, J=6.5 Hz, 3H), 0.58-0.41 (m, 4H); LCMS: C₃₀H₃₁F₄N₅O₂ requires: 569, found: m/z=570 [M+H]⁺.

### Example 577: 4-Cyclopropyl-5-fluoro-2-(3-(3-((4-methyl-4H-1,2,4-triazol-3-yl)methyl)oxetan-3-yl)phenyl)isoindolin-1-one

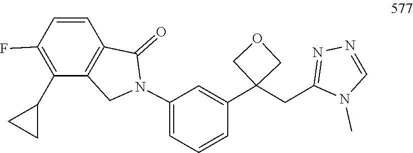 [SMILES: Cn1cnnc1CC1(c2cccc(N3Cc4c(ccc(F)c4C4CC4)C3=O)c2)COC1]

Step 1: Synthesis of ethyl 3-bromo-2-(bromomethyl)-4-fluorobenzoate A solution of ethyl 3-bromo-4-fluoro-2-methylbenzoate (1.0 g, 3.83 mmol, 1 eq.) in carbon tetrachloride (19 mL) was treated with BPO (93 mg, 0.38 mmol, 0.1 eq.) and N-bromosuccinimide (0.82 g, 4.62 mmol, 1.20 eq.) at rt. The reaction was heated at 80° C. for 12 hours. After cooling the mixture, the solvents were removed and the product was purified by chromatography A to afford the title compound (1.3 g, 100%) as a clear oil.

Step 2: Synthesis of 4-bromo-5-fluoro-2-(3-{3-[(4-methyl-1,2,4-triazol-3-yl)methyl]oxetan-3-yl}phenyl)-3H-isoindol-1-one The isoindoline formation with silver nitrate was carried out in a similar fashion as for Example Y using ethyl 3-bromo-2-(bromomethyl)-4-fluorobenzoate (240 mg, 0.71 mmol, 1 eq.) and Example R Example R (173 mg, 0.71 mmol, 1 eq.) as reactants afforded the title compound (92 mg, 28%) as a yellow solid.

Step 3: Synthesis of 4-cyclopropyl-5-fluoro-2-(3-{3-[(4-methyl-1,2,4-triazol-3-yl)methyl]oxetan-3-yl}phenyl)-3H-isoindol-1-one. 4-bromo-5-fluoro-2-(3-{3-[(4-methyl-1,2,4-triazol-3-yl)methyl]oxetan-3-yl}phenyl)-3H-isoindol-1-one (92 mg, 0.20 mmol, 1 eq) was coupled with cyclopropyl zinc bromide as in Step 1 of Example 339 to afford 25 mg (30%) of the title compounds as a yellow solid. 1H NMR (500 MHz, DMSO-d₆) δ 8.14 (s, 1H), 7.82-7.76 (m, 1H), 7.55 (dd, J=8.3, 4.5 Hz, 1H), 7.32-7.20 (m, 3H), 6.69 (dt, J=7.7, 1.1 Hz, 1H), 4.90 (d, J=6.2 Hz, 4H), 4.82 (d, J=6.1 Hz, 2H), 3.44 (s, 2H), 2.83 (s, 3H), 1.85 (tt, J=8.5, 5.5 Hz, 1H), 1.00-0.87 (m, 4H); LCMS: C₂₄H₂₃FN₄O₂ requires: 418, found: m/z=419 [M+H]⁺.

### Example 578: (S)-4-Cyclopropyl-6-((3-fluoropyrrolidin-1-yl)methyl)-2-(3-(3-((4-methyl-4H-1,2,4-triazol-3-yl)methyl)oxetan-3-yl)phenyl)-2,3-dihydro-1H-pyrrolo[3,4-c]pyri din-1-one

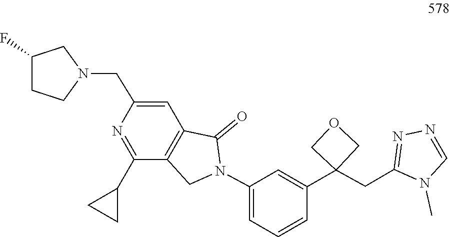 [SMILES: Cn1cnnc1CC1(c2cccc(N3Cc4c(cc(CN5CC[C@H](F)C5)nc4C4CC4)C3=O)c2)COC1]

Step 1: Synthesis of 6-chloro-4-cyclopropyl-2H,3H-pyrrolo[3,4-c]pyridin-1-one A solution of 4,6-dichloro-2H,3H-pyrrolo[3,4-c]pyridin-1-one (1.0 g, 4.9 mmol, 1 eq.), 2-cyclopropyl-4,4,5,5-tetramethyl-1,3,2-dioxaborolane (2.0 mL, 11 mmol, 2.2 eq.), [1,1′-bis(diphenylphosphino)ferrocene]dichloropalladium(II), complex with DCM (0.80 g, 1.0 mmol, 0.2 eq.) and potassium carbonate (3.4 g, 25 mmol, 5 eq.) in dioxane-water (10:1 v/v, 15 mL) was degassed and heated at 80° C. for 16 hours. After cooling the mixture, it was diluted with water and extracted with 15% isopropanol in chloroform (3×). The combined organic layers were dried, filtered and concentrated. The product was purified chromatography B to afford the title compound (0.44 g, 42%) as a white solid.

Step 2: 6-chloro-4-cyclopropyl-2-(3-{3-[(4-methyl-1,2,4-triazol-3-yl)methyl]oxetan-3-yl}phenyl)-3H-pyrrolo[3,4-c]pyridin-1-one. The C—N coupling reaction with potassium phosphate was carried out in a similar fashion as for Example 386 using 3-{[3-(3-bromophenyl)oxetan-3-yl]methyl}-4-methyl-1,2,4-triazole (2.8 g, 9.2 mmol, 1 eq.) and 6-chloro-4-cyclopropyl-2H,3H-pyrrolo[3,4-c]pyridin-1-one (1.9 g, 9.2 mmol, 1 eq.) as reactants afforded the title compound (2.3 g, 58%) as a brown solid.

Step 3: Synthesis of 4-cyclopropyl-6-ethenyl-2-(3-{3-[(4-methyl-1,2,4-triazol-3-yl)methyl]oxetan-3-yl}phenyl)-3H-pyrrolo[3,4-c]pyridin-1-one. The Stille coupling reaction was carried out in a similar fashion as for Example 459 using 6-chloro-4-cyclopropyl-2-(3-{3-[(4-methyl-1,2,4-triazol-3-yl)methyl]oxetan-3-yl}phenyl)-3H-pyrrolo[3,4-c]pyridin-1-one (0.70 g, 1.60 mmol, 1 eq.) as reactant afforded the title compound (0.38 g, 56%) as a clear oil.

Step 4: Synthesis of 4-cyclopropyl-2-(3-{3-[(4-methyl-1,2,4-triazol-3-yl)methyl]-oxetan-3-yl}phenyl)-1-oxo-3H-pyrrolo[3,4-c]pyridine-6-carbaldehyde. A solution of 4-cyclo-propyl-6-ethenyl-2-(3-{3-[(4-methyl-1,2,4-triazol-3-yl)methyl]oxetan-3-yl}phenyl)-3H-pyrrolo-[3,4-c]pyridin-1-one (0.38 g, 0.90 mmol, 1 eq.), 4-methylmorpholin-4-ium-4-olate (0.63 g, 5.4 mmol, 6 eq.), dipotassium dioxoosmiumbis(olate) dihydrate (33 mg, 0.090 mmol, 0.1 eq.) in THF (6.6 mL) and water (2.3 mL) was stirred at 50° C. for 4 h. The reaction was treated with sodium periodate (0.38 g, 1.8 mmol, 2 eq.) and stirred for 3 h at rt. The suspension was filtered and purified by chromatography C to afford the title compound (0.10 g, 27%) as a white solid.

Step 5: Synthesis of (S)-4-cyclopropyl-6-((3-fluoropyrrolidin-1-yl)methyl)-2-(3-(3-((4-methyl-4H-1,2,4-triazol-3-yl)methyl)oxetan-3-yl)phenyl)-2,3-dihydro-1H-pyrrolo[3,4-c]pyridin-1-one. The reductive animation reaction with sodium triacetoxyborohydride was carried out in a similar fashion as for Example 447 using 4-cyclopropyl-2-(3-{3-[(4-methyl-1,2,4-triazol-3-yl)methyl]oxetan-3-yl}phenyl)-1-oxo-3H-pyrrolo[3,4-c]pyridine-6-carbaldehyde (50 mg, 0.12 mmol, 1 eq.) and (3S)-3-fluoropyrrolidine hydrochloride (58 mg, 0.47 mmol, 4 eq.) as reactants afforded the title compound (38 mg, 65%) as an off-white solid, ¹H NMR (500 MHz, DMSO-d₆) δ 8.21 (s, 1H), 7.95 (dd, J=8.1, 2.1 Hz, 1H), 7.50-7.42 (m, 2H), 7.37 (t, J=8.0 Hz, 1H), 6.78 (dd, J=7.3, 1.5 Hz, 1H), 5.31-5.15 (m, 1H), 5.11 (s, 2H), 4.97 (d, J=6.0 Hz, 2H), 4.91 (d, J=6.1 Hz, 2H), 3.79 (s, 2H), 3.52 (s, 2H), 2.93 (s, 3H), 2.90-2.82 (m, 1H), 2.76-2.62 (m, 1H), 2.42 (q, J=8.2 Hz, 2H), 2.26-2.11 (m, 2H), 1.91 (ddt, J=30.0, 14.1, 6.9 Hz, 1H), 1.15-1.05 (m, 4H); LCMS: C₂₈H₃₁FN₆O₂ requires: 502, found: m/z=503 [M+H]⁺.

### Example 579: 4-Cyclopropyl-2-(3-(3-((4-methyl-4H-1,2,4-triazol-3-yl)methyl)oxetan-3-yl)phenyl)-6-(pyrrolidin-1-ylmethyl)-2,3-dihydro-1H-pyrrolo[3,4-c]pyridin-1-one

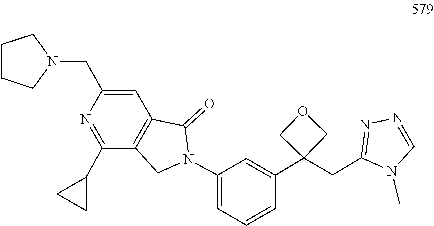 [SMILES: Cn1cnnc1CC1(c2cccc(N3Cc4c(cc(CN5CCCC5)nc4C4CC4)C3=O)c2)COC1]

The reductive amination was carried out in a similar fashion as for Example 447 using 4-cyclopropyl-2-(3-{3-[(4-methyl-1,2,4-triazol-3-yl)methyl]oxetan-3-yl}phenyl)-1-oxo-3H-pyrrolo[3,4-c]pyridine-6-carbaldehyde (50 mg, 0.12 mmol, 1 eq.) and pyrrolidine (25 mg, 0.35 mmol, 3 eq.) as reactants afforded the title compound (14 mg, 26%) as an off-white solid, ¹H NMR (500 MHz, DMSO-d₆) δ 8.21 (s, 1H), 7.95 (dd, J=8.3, 2.2 Hz, 1H), 7.49-7.42 (m, 2H), 7.37 (t, J=7.9 Hz, 1H), 6.80-6.76 (m, 1H), 5.10 (s, 2H), 4.97 (d, J=6.0 Hz, 2H), 4.91 (d, J=6.1 Hz, 2H), 3.75 (s, 2H), 3.52 (s, 2H), 2.93 (s, 3H), 2.60-2.30 (m, 4H), 2.23-2.18 (m, 1H), 1.73 (p, J=3.0 Hz, 4H), 1.10 (tt, J=8.0, 2.8 Hz, 4H); LCMS: C₂₈H₃₂N₆O₂ requires: 484, found: m/z=485 [M+H]⁺.

### Example 580: 2-(3-(3-((4-Methyl-4H-1,2,4-triazol-3-yl)methyl)oxetan-3-yl)phenyl)-6-(2,2,2-trifluoro-1-hydroxyethyl)-4-(trifluoromethyl)isoindolin-1-one

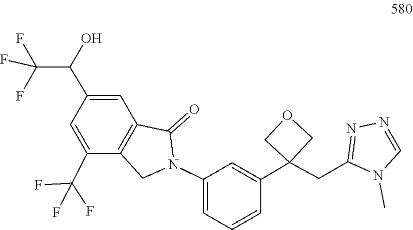 [SMILES: Cn1cnnc1CC1(c2cccc(N3Cc4c(cc(C(O)C(F)(F)F)cc4C(F)(F)F)C3=O)c2)COC1]

A solution of Example Z (29 mg, 0.064 mmol, 1 eq.) and potassium carbonate (4.5 mg, 0.032 mmol, 0.5 eq.) in DMF (0.33 mL) was treated with trifluoromethyltrimethylsilane (0.11 mL, 0.077 mmol, 1.2 eq.) at rt. The mixture was stirred for 16 hours at rt and then neutralized with saturated NH₄Cl solution and extracted with 15% isopropanol in chloroform (3×). The combined organic layers were dried on magnesium sulfate, filtered and concentrated. Purification by chromatography B afforded the product as an off-white solid, ¹H NMR (500 MHz, DMSO-d₆) δ 8.20 (d, J=3.5 Hz, 2H), 8.14 (s, 1H), 7.89 (ddd, J=8.2, 2.2, 1.0 Hz, 1H), 7.42 (t, J=2.0 Hz, 1H), 7.37 (t, J=8.0 Hz, 1H), 7.31 (d, J=5.8 Hz, 1H), 6.79 (dt, J=7.8, 1.2 Hz, 1H), 5.60 (q, J=6.9 Hz, 1H), 5.18-5.14 (m, 2H), 4.98 (d, J=6.0 Hz, 2H), 4.90 (d, J=6.1 Hz, 2H), 3.53 (s, 2H), 2.92 (s, 3H); LCMS: C₂₄H₂₀F₆N₄O₃ requires: 526, found: m/z=527 [M+H]⁺.

### Example 581: 2-(3-(3-((4-Methyl-4H-1,2,4-triazol-3-yl)methyl)oxetan-3-yl)phenyl)-6-(2,2,2-trifluoro-1-(pyrrolidin-1-yl)ethyl)-4-(trifluoromethyl)isoindolin-1-one

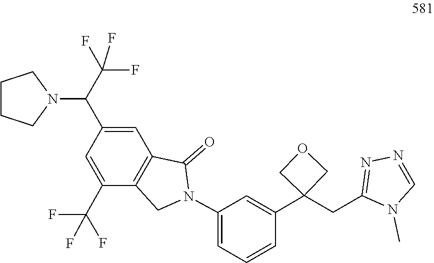 [SMILES: Cn1cnnc1CC1(c2cccc(N3Cc4c(cc(C(N5CCCC5)C(F)(F)F)cc4C(F)(F)F)C3=O)c2)COC1]

A solution of 2-(3-{3-[(4-methyl-1,2,4-triazol-3-yl)methyl]oxetan-3-yl}phenyl)-6-(2,2,2-trifluoro-1-hydroxyethyl)-4-(trifluoromethyl)-3H-isoindol-1-one (54 mg, 0.10 mmol, 1 eq.) and N,N-diisopropylethylamine (0.053 mL, 0.30 mmol, 3 eq.) in DCM (0.60 mL) was treated with methanesulfonyl chloride (0.0157 mL, 0.20 mmol, 2 eq.). The mixture was stirred at rt for 45 minutes, quenched with sat aq. sodium bicarbonate and extracted with DCM (3×). The combined organic layers were washed with sat. aq. sodium bicarbonate and water, dried (magnesium sulfate), filtered and concentrated to dryness. The residue was mixed with triethylamine (0.071 mL, 0.51 mmol, 5 eq.) and pyrrolidine (0.042 mL, 0.51 mml, 5 eq.) in DMF (0.40 mL). The mixture was stirred for 16 hours at rt and filtered. Purification by chromatography C afforded the product as an off-white solid. ¹H NMR (500 MHz, DMSO-d₆) δ 8.19 (s, 1H), 8.14 (s, 1H), 8.09 (s, 1H), 7.88 (dd, J=8.1, 2.1 Hz, 1H), 7.43-7.33 (m, 2H), 6.79 (dt, J=7.7, 1.2 Hz, 1H), 5.18-5.10 (m, 2H), 4.98 (d, J=6.0 Hz, 2H), 4.90 (d, J=6.1 Hz, 2H), 4.74 (q, J=8.0 Hz, 1H), 3.52 (s, 2H), 2.91 (s, 3H), 2.68-2.31 (m, 4H), 1.71 (p, J=3.4 Hz, 4H); LCMS: C₂₈H₂₇F₆N₅O₂ requires: 579, found: m/z=580 [M+H]⁺.

### Example 582: 2-(3-(3-((4-Methyl-4H-1,2,4-triazol-3-yl)methyl)oxetan-3-yl)phenyl)-6-(4,7-diazaspiro[2.5]octan-7-yl)-4-(trifluoromethyl)isoindolin-1-one

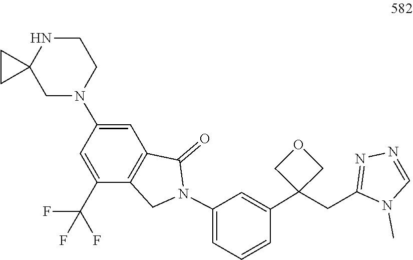 [SMILES: Cn1cnnc1CC1(c2cccc(N3Cc4c(cc(N5CCNC6(CC6)C5)cc4C(F)(F)F)C3=O)c2)COC1]

Step 1: Synthesis of tert-butyl 7-[2-(3-{3-[(4-methyl-1,2,4-triazol-3-yl)methyl]oxetan-3-yl}phenyl)-3-oxo-7-(trifluoromethyl)-1H-isoindol-5-yl]-4,7-diazaspiro[2.5]octane-4-carboxylate. The C—N coupling reaction with cesium carbonate was carried out in a similar fashion as for Example 386 using 6-bromo-2-(3-{3-[(4-methyl-1,2,4-triazol-3-yl)methyl]oxetan-3-yl}phenyl)-4-(trifluoromethyl)-3H-isoindol-1-one (400 mg, 0.79 mmol, 1 eq.) and 4,7-diaza-spiro[2.5]octane-4-carboxylic acid tert-butyl ester (202 mg, 0.95 mmol, 1.2 eq.) as reactants afforded the title compound (360 mg, 72%) as an off-white solid.

Step 2: Synthesis of 6-{4,7-diazaspiro[2.5]octan-7-yl}-2-(3-{3-[(4-methyl-1,2,4-triazol-3-yl)methyl]oxetan-3-yl}phenyl)-4-(trifluoromethyl)-3H-isoindol-1-one. The deprotection reaction was carried out in a similar fashion as for Step 10 of Example G using tert-butyl 7-[2-(3-{3-[(4-methyl-1,2,4-triazol-3-yl)methyl]oxetan-3-yl}phenyl)-3-oxo-7-(trifluoromethyl)-1H-isoindol-5-yl]-4,7-diazaspiro[2.5]octane-4-carboxylate (360 mg, 0.57 mmol, 1 eq.) as reactant afforded the title compound (200 mg, 64%) as an off-white solid: ¹H NMR (500 MHz, DMSO-d₆) δ 8.19 (s, 1H), 7.93-7.87 (m, 1H), 7.48 (d, J=2.2 Hz, 1H), 7.41 (d, J=2.3 Hz, 1H), 7.38-7.31 (m, 2H), 6.76 (dt, J=7.9, 1.2 Hz, 1H), 4.99-4.92 (m, 4H), 4.89 (d, J=6.0 Hz, 2H), 3.52 (s, 2H), 3.24 (t, J=5.1 Hz, 2H), 3.14 (s, 2H), 2.91 (s, 5H), 2.32 (s, 1H), 0.59 (q, J=4.1 Hz, 2H), 0.50 (t, J=3.0 Hz, 2H); LCMS: C₂₈H₂₉F₃N₆O₂ requires: 538, found: m/z=539 [M+H]⁺.

### Example 583: 2-(3-(3-((4-Methyl-4H-1,2,4-triazol-3-yl)methyl)oxetan-3-yl)phenyl)-6-(4-methyl-4,7-diazaspiro[2.5]octan-7-yl)-4-(trifluoromethyl)isoindolin-1-one

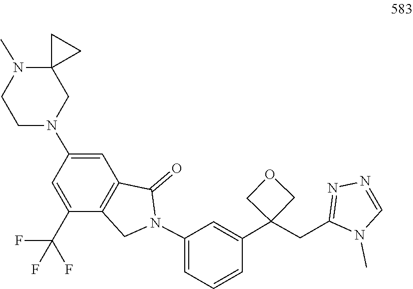 [SMILES: CN1CCN(c2cc3c(c(C(F)(F)F)c2)CN(c2cccc(C4(Cc5nncn5C)COC4)c2)C3=O)CC12CC2]

The reductive amination was carried out in a similar fashion as for Example 447 using 2-(3-(3-((4-methyl-4H-1,2,4-triazol-3-yl)methyl)oxetan-3-yl)phenyl)-6-(4,7-diazaspiro[2.5]-octan-7-yl)-4-(trifluoromethyl)isoindolin-1-one (76 mg, 0.14 mmol, 1 eq.) and formaldehyde (50% in water, 0.11 mL, 1.41 mmol, 10 eq.) as reactants afforded the title compound (56 mg, 74%) as an off-white solid. ¹H NMR (500 MHz, DMSO-d₆) δ 8.19 (s, 1H), 7.91 (dd, J=8.2, 2.2 Hz, 1H), 7.49 (d, J=2.2 Hz, 1H), 7.45 (d, J=2.3 Hz, 1H), 7.38-7.31 (m, 2H), 6.76 (dt, J=7.8, 1.2 Hz, 1H), 4.97 (m, 4H), 4.89 (d, J=6.0 Hz, 2H), 3.52 (s, 2H), 3.30 (s, 2H), 3.16 (s, 2H), 2.97 (s, 2H), 2.91 (s, 3H), 2.31 (s, 3H), 0.65 (q, J=4.3, 3.7 Hz, 2H), 0.62-0.54 (m, 2H); LCMS: C₂₉H₃₁F₃N₆O₂ requires: 552, found: m/z=553 [M+H]⁺.

### Example 584: 4-Cyclopropyl-2-(3-(3-((4-methyl-4H-1,2,4-triazol-3-yl)methyl)oxetan-3-yl)phenyl)-6-(l-(pyrrolidin-1-yl)ethyl)-2,3-dihydro-1H-pyrrolo[3,4-c]pyridin-1-one

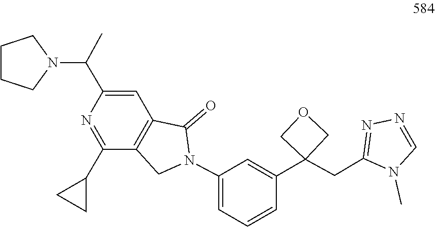 [SMILES: CC(c1cc2c(c(C3CC3)n1)CN(c1cccc(C3(Cc4nncn4C)COC3)c1)C2=O)N1CCCC1]

Step 1: Synthesis of 6-acetyl-4-cyclopropyl-2-(3-{3-[(4-methyl-1,2,4-triazol-3-yl)methyl]oxetan-3-yl}phenyl)-3H-pyrrolo[3,4-c]pyridin-1-one. The Stille coupling reaction was carried out in a similar fashion as Step 4 of Examples B using 6-chloro-4-cyclopropyl-2-(3-{3-[(4-methyl-1,2,4-triazol-3-yl)methyl]oxetan-3-yl}phenyl)-3H-pyrrolo[3,4-c]pyridin-1-one (0.50 g, 1.2 mmol, 1 eq.) as reactant afforded the title compound (0.37 g, 73%) as an off-white solid.

Step 2: Synthesis of (4-cyclopropyl-2-(3-(3-((4-methyl-4H-1,2,4-triazol-3-yl)methyl)oxetan-3-yl)phenyl)-6-(l-(pyrrolidin-1-yl)ethyl)-2,3-dihydro-1H-pyrrolo[3,4-c]pyridin-1-one. The reductive amination reaction with sodium cyanoborohydride was carried out in a similar fashion as for Example 459 using 6-acetyl-4-cyclopropyl-2-(3-{3-[(4-methyl-1,2,4-triazol-3-yl)methyl]oxetan-3-yl}phenyl)-3H-pyrrolo[3,4-c]pyridin-1-one (46 mg, 0.10 mmol, 1 eq.) and pyrrolidine (15 mg, 0.21 mmol, 2 eq.) as reactants afforded the title compound (20 mg, 39%) as an off-white solid. ¹H NMR (500 MHz, DMSO-d₆) δ 8.21 (s, 1H), 7.95 (dd, J=8.0, 2.1 Hz, 1H), 7.45 (s, 1H), 7.42 (d, J=2.0 Hz, 1H), 7.37 (t, J=8.0 Hz, 1H), 6.79 (dt, J=7.8, 1.3 Hz, 1H), 5.09 (s, 2H), 4.97 (d, J=6.0 Hz, 2H), 4.90 (d, J=6.2 Hz, 2H), 3.52 (s, 2H), 3.50 (d, J=6.7 Hz, 1H), 2.92 (s, 3H), 2.38 (s, 4H), 2.23-2.17 (m, 1H), 1.68 (t, J=4.1 Hz, 4H), 1.32 (d, J=6.6 Hz, 3H), 1.15-1.04 (m, 4H); LCMS: C₂₉H₃₄N₆O₂ requires: 498, found: m/z=499 [M+H]⁺.

### Example 585: 6-(1-(5-Azaspiro[2.4]heptan-5-yl)ethyl)-4-cyclopropyl-2-(3-(3-((4-methyl-4H-1,2,4-triazol-3-yl)methyl)oxetan-3-yl)phenyl)-2,3-dihydro-1H-pyrrolo[3,4-c]pyri din-1-one

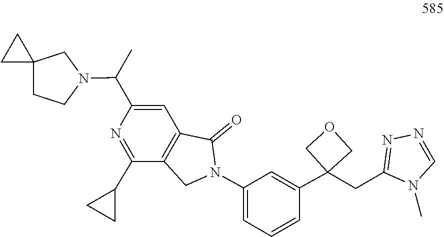 [SMILES: CC(c1cc2c(c(C3CC3)n1)CN(c1cccc(C3(Cc4nncn4C)COC3)c1)C2=O)N1CCC2(CC2)C1]

The reductive amination reaction with sodium cyanoborohydride was carried out in a similar fashion as for Example 459 using 6-acetyl-4-cyclopropyl-2-(3-{3-[(4-methyl-1,2,4-triazol-3-yl)methyl]oxetan-3-yl}phenyl)-3H-pyrrolo[3,4-c]pyridin-1-one (100 mg, 0.23 mmol, 1 eq.) and 5-azaspiro[2.4]heptane hydrochloride (60 mg, 0.45 mmol, 2 eq.) as reactants afforded the title compound (50 mg, 43%) as an off-white solid. 1H NMR (500 MHz, DMSO-d₆) δ 8.21 (s, 1H), 7.95 (dd, J=8.1, 2.1 Hz, 1H), 7.48 (s, 1H), 7.43 (t, J=1.9 Hz, 1H), 7.37 (t, J=8.0 Hz, 1H), 6.79 (d, J=7.5 Hz, 1H), 5.09 (s, 2H), 4.97 (d, J=6.0 Hz, 2H), 4.91 (d, J=6.1 Hz, 2H), 3.52 (s, 3H), 2.92 (s, 3H), 2.73 (q, J=7.7 Hz, 1H), 2.60-2.45 (m, 2H), 2.39 (d, J=8.8 Hz, 2H), 2.23-2.16 (m, 1H), 1.73 (ddd, J=18.2, 12.4, 6.1 Hz, 2H), 1.31 (d, J=6.7 Hz, 3H), 1.10 (td, J=10.9, 10.3, 3.7 Hz, 4H), 0.49 (d, J=8.0 Hz, 4H); LCMS: C₃₁H₃₆N₆O₂ requires: 524, found: m/z=525 [M+H]⁺.

### Example 586: 6-(1-(5-Azaspiro[2.3]hexan-5-yl)ethyl)-4-cyclopropyl-2-(3-(3-((4-methyl-4H-1,2,4-triazol-3-yl)methyl)oxetan-3-yl)phenyl)-2,3-dihydro-1H-pyrrolo[3,4-c]pyri din-1-one

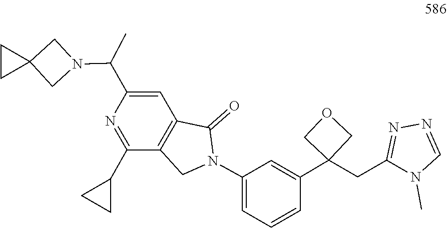 [SMILES: CC(c1cc2c(c(C3CC3)n1)CN(c1cccc(C3(Cc4nncn4C)COC3)c1)C2=O)N1CC2(CC2)C1]

The reductive amination reaction with sodium cyanoborohydride was carried out in a similar fashion as for Example 459 using 6-acetyl-4-cyclopropyl-2-(3-{3-[(4-methyl-1,2,4-triazol-3-yl)methyl]oxetan-3-yl}phenyl)-3H-pyrrolo[3,4-c]pyridin-1-one (100 mg, 0.23 mmol, 1 eq.) and 5-azaspiro[2.3]hexane (37 mg, 0.45 mmol, 2 eq.) as reactants afforded the title compound (46 mg, 39%) as an off-white solid, ¹H NMR (500 MHz, DMSO-d₆) δ 8.21 (s, 1H), 7.94 (dd, J=8.2, 2.1 Hz, 1H), 7.43 (d, J=2.5 Hz, 2H), 7.37 (t, J=7.9 Hz, 1H), 6.78 (dt, J=7.8, 1.2 Hz, 1H), 5.08 (s, 2H), 4.97 (d, J=6.1 Hz, 2H), 4.90 (d, J=6.0 Hz, 2H), 3.59 (q, J=6.6 Hz, 1H), 3.52 (s, 2H), 3.24 (s, 4H), 2.92 (s, 3H), 2.23-2.15 (m, 1H), 1.21-0.99 (m, 7H), 0.54-0.47 (m, 4H); LCMS: C₃₀H₃₄N₆O₂ requires: 510, found: m/z=511 [M+H]⁺.

### Example 587: 4-Cyclopropyl-2-(3-(3-((4-methyl-4H-1,2,4-triazol-3-yl)methyl)oxetan-3-yl)phenyl)-6-(l-(piperidin-1-yl)ethyl)-2,3-dihydro-1H-pyrrolo[3,4-c]pyridin-1-one

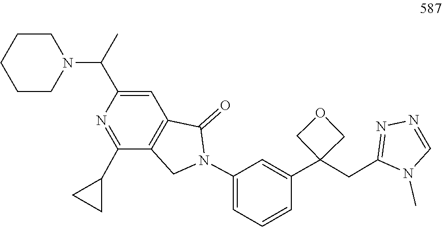 [SMILES: CC(c1cc2c(c(C3CC3)n1)CN(c1cccc(C3(Cc4nncn4C)COC3)c1)C2=O)N1CCCCC1]

The reductive amination reaction with sodium cyanoborohydride was carried out in a similar fashion as for Example 459 using 6-acetyl-4-cyclopropyl-2-(3-{3-[(4-methyl-1,2,4-triazol-3-yl)methyl]oxetan-3-yl}phenyl)-3H-pyrrolo[3,4-c]pyridin-1-one (100 mg, 0.23 mmol, 1 eq.) and piperidine (38 mg, 0.45 mmol, 2 eq.) as reactants afforded the title compound (14.3 mg, 12%) as an off-white solid. ¹H NMR (500 MHz, DMSO-d₆) δ 8.21 (s, 1H), 7.94 (dd, J=8.2, 2.1 Hz, 1H), 7.43 (d, J=2.5 Hz, 2H), 7.37 (t, J=7.9 Hz, 1H), 6.78 (dt, J=7.8, 1.2 Hz, 1H), 5.08 (s, 2H), 4.97 (d, J=6.1 Hz, 2H), 4.90 (d, J=6.0 Hz, 2H), 3.59 (q, J=6.6 Hz, 1H), 3.52 (s, 2H), 3.24 (s, 4H), 2.92 (s, 3H), 2.23-2.15 (m, 1H), 1.21-0.99 (m, 7H), 0.54-0.47 (m, 4H); LCMS: C₃₀H₃₄N₆O₂ requires: 510, found: m/z=511 [M+H]⁺.

### Example 588: 6-((2-Methyl-3-(trifluoromethyl)pyrrolidin-1-yl)methyl)-2-(3-(3-((4-methyl-4H-1,2,4-triazol-3-yl)methyl)oxetan-3-yl)phenyl)-4-(trifluoromethyl)isoindolin-1-one

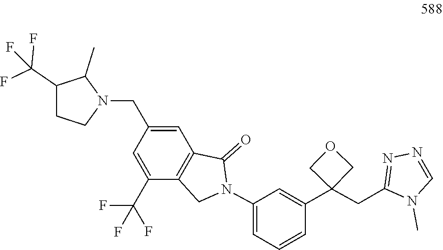 [SMILES: CC1C(C(F)(F)F)CCN1Cc1cc2c(c(C(F)(F)F)c1)CN(c1cccc(C3(Cc4nncn4C)COC3)c1)C2=O]

The reductive amination reaction with sodium cyanoborohydride was carried out in a similar fashion as for Example 459 using 2-(3-{3-[(4-methyl-1,2,4-triazol-3-yl)methyl]oxetan-3-yl}phenyl)-3-oxo-7-(trifluoromethyl)-1H-isoindole-5-carbaldehyde (100 mg, 0.22 mmol, 1 eq.) and 2-methyl-3-(trifluoromethyl)pyrrolidine hydrochloride (83 mg, 0.44 mmol, 2 eq.) as reactants afforded the title compound (70 mg, 54%) as an off-white solid. 1H NMR (500 MHz, DMSO-d₆) δ 8.20 (s, 1H), 8.00 (s, 1H), 7.94 (s, 1H), 7.92-7.86 (m, 1H), 7.43-7.33 (m, 2H), 6.78 (dt, J=7.6, 1.3 Hz, 1H), 5.11 (s, 2H), 4.97 (d, J=6.0 Hz, 2H), 4.90 (d, J=6.1 Hz, 2H), 4.10 (d, J=14.0 Hz, 1H), 3.55-3.45 (m, 1H), 3.52 (s, 2H), 3.08 (dt, J=19.1, 8.8 Hz, 1H), 2.91 (s, 3H), 2.87-2.76 (m, 1H), 2.25 (q, J=8.8 Hz, 1H), 2.06-1.98 (m, 1H), 1.78 (ddd, J=16.9, 12.4, 8.1 Hz, 1H), 1.27 (d, J=6.0 Hz, 1H), 1.23-1.12 (m, 3H); LCMS: C29H29F₆N₅O₂ requires: 593, found: m/z=594 [M+H]⁺.

### Example 589: 2-Cyclopropyl-N-(3-(3-((4-methyl-4H-1,2,4-triazol-3-yl)methyl)oxetan-3-yl)phenyl)-6-(piperidin-1-ylmethyl)pyrimidine-4-carboxamide

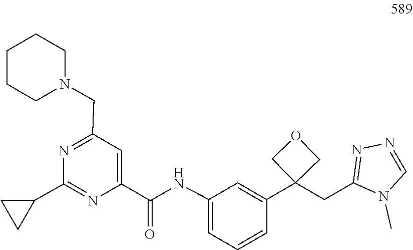 [SMILES: Cn1cnnc1CC1(c2cccc(NC(=O)c3cc(CN4CCCCC4)nc(C4CC4)n3)c2)COC1]

Step 1: Synthesis of ethyl 2-cyclopropyl-6-(piperidin-1-ylmethyl)pyrimidine-4-carboxylate A solution of ethyl 6-(bromomethyl)-2-cyclopropylpyrimidine-4-carboxylate (77 mg, 0.27 mmol, 1 eq.) and N,N-diisopropylethylamine (0.070 g, 0.54 mmol, 2 eq.) in acetonitrile (0.77 mL) was treated with piperidine (0.025 g, 0.30 mmol, 1.1 eq.) at rt. The mixture was stirred for 2 h at rt. Solvent was removed to yield the title compound.

Step 2: Synthesis of 2-cyclopropyl-6-(piperidin-1-ylmethyl)pyrimidine-4-carboxylic acid A solution of ethyl 2-cyclopropyl-6-(piperidin-1-ylmethyl)pyrimidine-4-carboxylate (0.27 mmol, 1 eq.) in THF (1.0 mL) was treated with an aqueous solution of lithium hydroxide (1M, 0.54 mL, 0.54 mmol, 2 eq.) at rt. The mixture was stirred for 2 h at rt and quenched with hydrochloric acid (2M, 0.27 mL, 0.54 mmol, 2 eq.). THF was removed and the mixture was dried on a lyophilizer to yield the title compound.

Step 3: Synthesis of 2-cyclopropyl-N-(3-(3-((4-methyl-4H-1,2,4-triazol-3-yl)methyl)oxetan-3-yl)phenyl)-6-(piperidin-1-ylmethyl)pyrimidine-4-carboxamide. The amide bond formation reaction was carried out in a similar fashion as for Example 56 using the 2-cyclopropyl-6-(piperidin-1-ylmethyl)pyrimidine-4-carboxylic acid (0.27 mmol, 1 eq.) and Example R (0.067 g, 0.27 mmol, 1 eq.) as reactants afforded the title compound (44 mg, 33%) as an off-white solid: ¹H NMR (500 MHz, DMSO-d₆) δ 10.42 (s, 1H), 8.21 (s, 1H), 7.86 (s, 1H), 7.79 (dd, J=7.9, 2.0 Hz, 1H), 7.50 (t, J=1.9 Hz, 1H), 7.30 (t, J=7.9 Hz, 1H), 6.70 (dt, J=7.7, 1.3 Hz, 1H), 4.95 (d, J=6.0 Hz, 2H), 4.87 (d, J=6.0 Hz, 2H), 3.60 (s, 2H), 3.50 (s, 2H), 2.94 (s, 3H), 2.41 (t, J=5.2 Hz, 5H), 1.55 (p, J=5.6 Hz, 4H), 1.43 (d, J=6.5 Hz, 2H), 1.18 (tt, J=5.4, 2.7 Hz, 2H), 1.17-1.08 (m, 2H); LCMS: C₂₇H₃₃N₇O₂ requires: 487, found: m/z=488 [M+H]⁺.

### Example 590: (R)-6-(Hexahydropyrrolo[1,2-a]pyrazin-2(1H)-yl)-2-(3-(3-((4-methyl-4H-1,2,4-triazol-3-yl)methyl)oxetan-3-yl)phenyl)-4-(trifluoromethyl)isoindolin-1-one

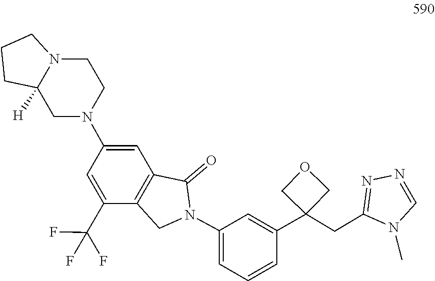 [SMILES: Cn1cnnc1CC1(c2cccc(N3Cc4c(cc(N5CCN6CCC[C@@H]6C5)cc4C(F)(F)F)C3=O)c2)COC1]

The C—N coupling reaction with cesium carbonate was carried out in a similar fashion as for Example 386 using 6-bromo-2-(3-{3-[(4-methyl-1,2,4-triazol-3-yl)methyl]oxetan-3-yl}phenyl)-4-(trifluoromethyl)-3H-isoindol-1-one (90 mg, 0.18 mmol, 1 eq.) and (R)-1,4-diazabicyclo[4.3.0]nonane (27 mg, 0.21 mmol, 1.2 eq.) as reactants afforded the title compound (49 mg, 50%) as an off-white solid: ¹H NMR (500 MHz, DMSO-d₆) δ 8.20 (s, 1H), 7.91 (dd, J=8.3, 2.0 Hz, 1H), 7.54 (d, J=2.3 Hz, 1H), 7.46 (d, J=2.3 Hz, 1H), 7.38-7.31 (m, 2H), 6.76 (d, J=10.0 Hz, 1H), 4.97 (d, J=5.9 Hz, 4H), 4.90 (d, J=6.1 Hz, 2H), 4.05-3.99 (m, 1H), 3.89-3.82 (m, 1H), 3.52 (s, 2H), 3.10 (d, J=11.1 Hz, 1H), 3.05 (td, J=8.2, 2.5 Hz, 1H), 2.91 (s, 3H), 2.86 (dd, J=11.7, 3.4 Hz, 1H), 2.29-2.24 (m, 1H), 2.09 (q, J=8.9 Hz, 2H), 1.89-1.85 (m, 1H), 1.79-1.69 (m, 2H), 1.47-1.35 (m, 2H); LCMS: C₂₉H₃₁F₃N₆O₂ requires: 553, found: m/z=554 [M+H]⁺.

### Example 591: (S)-6-(Hexahydropyrrolo[1,2-a]pyrazin-2(1H)-yl)-2-(3-(3-((4-methyl-4H-1,2,4-triazol-3-yl)methyl)oxetan-3-yl)phenyl)-4-(trifluoromethyl)isoindolin-1-one

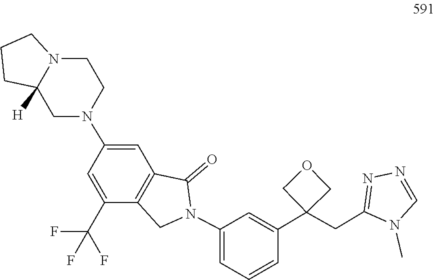 [SMILES: Cn1cnnc1CC1(c2cccc(N3Cc4c(cc(N5CCN6CCC[C@H]6C5)cc4C(F)(F)F)C3=O)c2)COC1]

The C—N coupling reaction with cesium carbonate was carried out in a similar fashion as for Example 386 using 6-bromo-2-(3-{3-[(4-methyl-1,2,4-triazol-3-yl)methyl]oxetan-3-yl}phenyl)-4-(trifluoromethyl)-3H-isoindol-1-one (80 mg, 0.16 mmol, 1 eq.) and (S)-1,4-diazabicyclo[4.3.0]nonane (24 mg, 0.19 mmol, 1.2 eq.) as reactants afforded the title compound (34 mg, 38%) as an off-white solid: ¹H NMR (500 MHz, DMSO-d₆) δ 8.20 (s, 1H), 7.91 (dd, J=8.1, 2.1 Hz, 1H), 7.54 (d, J=2.2 Hz, 1H), 7.46 (d, J=2.3 Hz, 1H), 7.38-7.31 (m, 2H), 6.76 (dt, J=7.4, 1.3 Hz, 1H), 4.97 (d, J=5.9 Hz, 4H), 4.90 (d, J=6.1 Hz, 2H), 4.05-3.99 (m, 1H), 3.85 (d, J=11.6 Hz, 1H), 3.52 (s, 2H), 3.14-3.08 (m, 1H), 3.05 (td, J=8.2, 2.5 Hz, 1H), 2.91 (s, 3H), 2.90-2.83 (m, 1H), 2.24 (dd, J=11.3, 3.3 Hz, 1H), 2.09 (q, J=8.8 Hz, 2H), 1.91-1.83 (m, 1H), 1.72 (dtd, J=16.4, 9.4, 8.2, 3.4 Hz, 2H), 1.47-1.35 (m, 2H); LCMS: C₂₉H₃₁F₃N₆O₂ requires: 553, found: m/z=554 [M+H]⁺.

### Example 592a and 592b: (R)-6-(1-(5-azaspiro[2.4]heptan-5-yl)ethyl)-2-cyclopropyl-N-(3-(3-((4-methyl-4H-1,2,4-triazol-3-yl)methyl)oxetan-3-yl)phenyl)pyrimidine-4-carboxamide and (S)-6-(1-(5-azaspiro[2.4]heptan-5-yl)ethyl)-2-cyclopropyl-N-(3-(3-((4-methyl-4H-1,2,4-triazol-3-yl)methyl)oxetan-3-yl)phenyl)pyrimidine-4-carboxamide

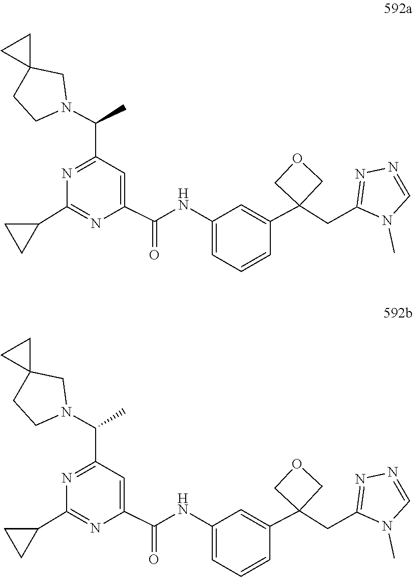 [SMILES: C[C@@H](c1cc(C(=O)Nc2cccc(C3(Cc4nncn4C)COC3)c2)nc(C2CC2)n1)N1CCC2(CC2)C1 ; C[C@H](c1cc(C(=O)Nc2cccc(C3(Cc4nncn4C)COC3)c2)nc(C2CC2)n1)N1CCC2(CC2)C1]

Step 1: Synthesis of 6-acetyl-2-cyclopropylpyrimidine-4-carboxylic acid. A solution of ethyl 6-acetyl-2-cyclopropylpyrimidine-4-carboxylate (0.16 g, 0.68 mmol, 1 eq.) in THF (2.4 mL) was treated with an aqueous solution of lithium hydroxide (1M, 1.0 mL, 1.0 mmol, 1.5 eq.) at rt. The mixture was stirred for 2 h at rt and quenched with hydrochloric acid (2M, 0.51 mL, 1.0 mmol, 1.5 eq.). THF was removed and the mixture was dried on a lyophilizer to yield the title compound.

Step 2: Synthesis of 2-cyclopropyl-N-(3-(3-((4-methyl-4H-1,2,4-triazol-3-yl)-methyl)oxetan-3-yl)phenyl)-6-(piperidin-1-ylmethyl)pyrimidine-4-carboxamide. The amide bond formation reaction was carried out in a similar fashion as for Example 56 using 6-acetyl-2-cyclopropylpyrimidine-4-carboxylic acid (0.68 mmol, 1 eq.) and Example R (0.18 g, 0.75 mmol, 1.1 eq.) as reactants afforded the title compound (75 mg, 26%) as an off-white solid.

Step 3: Synthesis of 6-(1-(5-azaspiro[2.4]heptan-5-yl)ethyl)-2-cyclopropyl-N-(3-(3-((4-methyl-4H-1,2,4-triazol-3-yl)methyl)oxetan-3-yl)phenyl)pyrimidine-4-carboxamide. The reductive amination reaction with sodium cyanoborohydride was carried out in a similar fashion as for Example 459 using 6-acetyl-2-cyclopropyl-N-(3-{3-[(4-methyl-1,2,4-triazol-3-yl)methyl]oxetan-3-yl}phenyl)pyrimidine-4-carboxamide (75 mg, 0.17 mmol, 1 eq.) and 5-azaspiro[2.4]heptane hydrochloride (46 mg, 0.35 mmol, 2 eq.) as reactants afforded the title compound (26 mg, 29%) as an off-white solid.

Step 4: The enantiomers (0.026 g) were separated using chiral chromatography on a CHIRALPAK AZ column with CO₂ and methanol as mobile phase to afford (R)-6-(1-(5-azaspiro[2,4]heptan-5-yl)ethyl)-2-cyclopropyl-N-(3-(3-((4-methyl-4H-1,2,4-triazol-3-yl)methyl)oxetan-3-yl)phenyl)pyrimidine-4-carboxamide (7.0 mg, shorter retention time) and (S)-6-(1-(5-azaspiro[2.4]heptan-5-yl)ethyl)-2-cyclopropyl-N-(3-(3-((4-methyl-4H-1,2,4-triazol-3-yl)methyl)oxetan-3-yl)phenyl)pyrimidine-4-carboxamide (3.0 mg, longer retention time) as off-white solids.

6-[(1S)-1-{5-azaspiro[2.4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one: ¹H NMR (500 MHz, DMSO-d₆) δ 8.21 (s, 1H), 7.99 (s, 1H), 7.73 (dd, J=7.9, 2.0 Hz, 1H), 7.35 (s, 2H), 6.69 (d, J=7.7 Hz, 1H), 5.12-5.03 (m, 4H), 3.66 (s, 2H), 3.59 (q, J=6.7 Hz, 1H), 2.93 (d, J=8.0 Hz, 1H), 2.90 (s, 3H), 2.73 (q, J=7.8 Hz, 1H), 2.66 (d, J=9.1 Hz, 1H), 2.53 (d, J=9.0 Hz, 1H), 2.42 (tt, J=8.3, 4.6 Hz, 1H), 1.87 (q, J=6.7 Hz, 2H), 1.45 (d, J=6.6 Hz, 3H), 1.29 (p, J=4.0 Hz, 2H), 1.21-1.14 (m, 2H), 0.58 (q, J=7.9, 6.0 Hz, 4H); LCMS: C₂₉H₃₅N₇O₂ requires: 513, found: m/z=514 [M+H]⁺.

6-[(1R)-1-{5-azaspiro[2.4]heptan-5-yl}ethyl]-2-(3-{3-[(R)-fluoro(4-methyl-1,2,4-triazol-3-yl)methyl]oxetan-3-yl}phenyl)-4-(trifluoromethyl)-3H-isoindol-1-one: ¹H NMR (500 MHz, DMSO-d₆) δ 8.21 (s, 1H), 7.99 (s, 1H), 7.73 (dd, J=7.9, 2.0 Hz, 1H), 7.39-7.32 (m, 2H), 6.69 (d, J=7.8 Hz, 1H), 5.12-5.04 (m, 4H), 3.66 (s, 2H), 3.59 (q, J=6.7 Hz, 1H), 2.94-2.87 (m, 1H), 2.90 (s, 3H), 2.73 (q, J=7.7 Hz, 1H), 2.66 (d, J=9.1 Hz, 1H), 2.52 (d, J=9.1 Hz, 1H), 2.42 (tt, J=8.3, 4.6 Hz, 1H), 1.86 (hept, J=6.7, 6.2 Hz, 2H), 1.45 (d, J=6.7 Hz, 3H), 1.34-1.25 (m, 2H), 1.17 (dq, J=7.1, 3.7 Hz, 2H), 0.59 (d, J=4.5 Hz, 4H); LCMS: C₂₉H₃₅N₇O₂ requires: 513, found: m/z=514 [M+H]⁺.

### Example 593: 2-Cyclopropyl-N-(3-(3-((4-methyl-4H-1,2,4-triazol-3-yl)methyl)oxetan-3-yl)phenyl)-6-(l-(pyrrolidin-1-yl)ethyl)pyrimidine-4-carboxamide

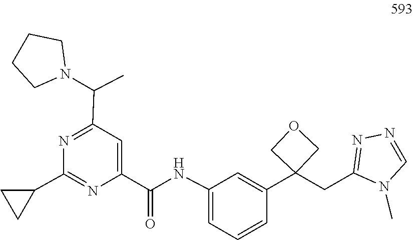 [SMILES: CC(c1cc(C(=O)Nc2cccc(C3(Cc4nncn4C)COC3)c2)nc(C2CC2)n1)N1CCCC1]

The reductive amination reaction with sodium cyanoborohydride was carried out in a similar fashion as for Example 459 using 6-acetyl-2-cyclopropyl-N-(3-{3-[(4-methyl-1,2,4-triazol-3-yl)methyl]oxetan-3-yl}phenyl)pyrimidine-4-carboxamide (22 mg, 0.051 mmol, 1 eq.) and pyrrolidine (7.3 mg, 0.10 mmol, 2 eq.) as reactants afforded the title compound (5.1 mg, 21%) as an off-white solid. ¹H NMR (500 MHz, DMSO-d₆) δ 10.41 (s, 1H), 8.21 (s, 1H), 7.83 (s, 1H), 7.81-7.75 (m, 1H), 7.47 (t, J=2.0 Hz, 1H), 7.30 (t, J=7.9 Hz, 1H), 6.74-6.68 (m, 1H), 4.95 (d, J=5.9 Hz, 2H), 4.87 (d, J=6.0 Hz, 2H), 3.50 (s, 2H), 3.44 (q, J=6.8 Hz, 1H), 2.93 (s, 3H), 2.44-2.32 (m, 5H), 1.71 (p, J=3.2 Hz, 4H), 1.33 (d, J=6.7 Hz, 3H), 1.19 (td, J=5.1, 2.2 Hz, 2H), 1.17-1.10 (m, 2H); LCMS: C₂₇H₃₃N₇O₂ requires: 487, found: m/z=488 [M+H]⁺.

### Example 594: 6-(3-(Difluoromethyl)azetidin-1-yl)-2-(3-(3-((4-methyl-4H-1,2,4-triazol-3-yl)methyl)oxetan-3-yl)phenyl)-4-(trifluoromethyl)isoindolin-1-one

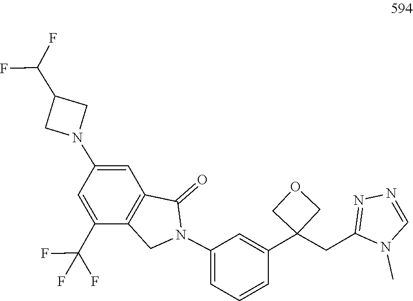 [SMILES: Cn1cnnc1CC1(c2cccc(N3Cc4c(cc(N5CC(C(F)F)C5)cc4C(F)(F)F)C3=O)c2)COC1]

The C—N coupling reaction with cesium carbonate was carried out in a similar fashion as for Example 386 using 6-bromo-2-(3-{3-[(4-methyl-1,2,4-triazol-3-yl)methyl]oxetan-3-yl}phenyl)-4-(trifluoromethyl)-3H-isoindol-1-one (50 mg, 0.099 mmol, 1 eq.) and 3-(difluoromethyl)azetidine hydrochloride (24 mg, 0.17 mmol, 1.7 eq.) as reactants afforded the title compound (29 mg, 55%) as an off-white solid: 1H NMR (500 MHz, DMSO-d₆) δ 8.20 (s, 1H), 7.90 (dd, J=8.1, 2.1 Hz, 1H), 7.40-7.31 (m, 2H), 7.02 (q, J=2.1 Hz, 2H), 6.76 (d, J=7.9 Hz, 1H), 6.41 (dt, J=55, 4.5 Hz, 1H), 5.00-4.86 (m, 6H), 4.11 (t, J=8.3 Hz, 2H), 3.94 (dd, J=8.1, 5.4 Hz, 2H), 3.52 (s, 2H), 3.40-3.22 (m, 1H), 2.91 (s, 3H); LCMS: C₂₆H₂₄F₅N₅O₂ requires: 533, found: m/z=534 [M+H]⁺.

### Example 595: 6-(Difluoromethoxy)-2-(3-(3-((4-methyl-4H-1,2,4-triazol-3-yl)methyl)oxetan-3-yl)phenyl)-4-(trifluoromethyl)isoindolin-1-one

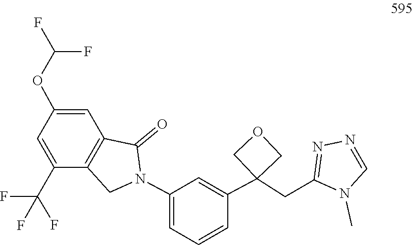 [SMILES: Cn1cnnc1CC1(c2cccc(N3Cc4c(cc(OC(F)F)cc4C(F)(F)F)C3=O)c2)COC1]

Step 1: Synthesis of methyl 5-(difluoromethoxy)-2-methyl-3-(trifluoromethyl)-benzoate A mixture of methyl 5-hydroxy-2-methyl-3-(trifluoromethyl)benzoate (770 mg, 3.3 mmol, 1 eq.), methyl 2-chloro-2,2-difluoroacetate (1.0 g, 7.2 mmol, 2.2 eq.) and cesium carbonate (2.4 g, 7.2 mmol, 2.2 eq.) in butan-2-one (4.0 mL) was heated at 80° C. for 48 h. The mixture was cooled to rt and filtered. The filtrate was concentrated under vacuum. The residue was purified by chromatography A to afford the title compound (520 mg, 55%) as a colorless oil.

Step 2: Synthesis of methyl 2-(bromomethyl)-5-(difluoromethoxy)-3-(trifluoromethyl)benzoate A mixture of methyl 5-(difluoromethoxy)-2-methyl-3-(trifluoromethyl)benzoate (520 mg, 1.8 mmol), N-bromosuccinimide (420 mg, 2.4 mmol) and BPO (131 mg, 0.51 mmol) in carbon tetrachloride (15 mL) was heated at 80° C. for 16 h. The solids were filtered out and the filtrate was concentrated under vacuum. The residue was purified by chromatography A to afford the title compound (330 mg, 49%) as a colorless syrup.

Step 3: Synthesis of 6-(difluoromethoxy)-4-(trifluoromethyl)isoindolin-1-one A mixture of 2-(bromomethyl)-5-(difluoromethoxy)-3-(trifluoromethyl)benzoate (330 mg, 0.91 mmol) in ammonia in methanol (7M, 5.0 mL) was stirred for 2 h at 25° C. The resulting mixture was concentrated under vacuum. The residue was purified by chromatography A to afford the title compound (200 mg, 84%) as an off-white solid.

Step 4: Synthesis of 6-(difluoromethoxy)-2-(3-(3-((4-methyl-4H-1,2,4-triazol-3-yl)methyl)oxetan-3-yl)phenyl)-4-(trifluoromethyl)isoindolin-1-one. The C—N coupling reaction with cesium carbonate was carried out in a similar fashion as for Example 386 using 3-{[3-(3-bromophenyl)oxetan-3-yl]methyl}-4-methyl-1,2,4-triazole (50 mg, 0.16 mmol, 1 eq.) and 6-(difluoromethoxy)-4-(trifluoromethyl)-2,3-dihydroisoindol-1-one (48 mg, 0.18 mmol, 1.1 eq.) as reactants afforded the title compound (15 mg, 18%) as an off-white solid: ¹H NMR (500 MHz, DMSO-d₆) δ 1H NMR (500 MHz, DMSO-d6) δ 8.20 (s, 1H), 7.92-7.83 (m, 3H), 7.71-7.33 (m, 2H), 6.80 (d, J=7.6 Hz, 1H), 5.13 (s, 2H), 4.98 (d, J=6.0 Hz, 2H), 4.90 (d, J=6.1 Hz, 2H), 3.53 (s, 2H), 2.93 (s, 3H); LCMS: C₂₃H₁₉F₅N₅O₃ requires: 494, found: m/z=495 [M+H]⁺.

### Example 596: 5-Methyl-N-(3-(3-((4-methyl-4H-1,2,4-triazol-3-yl)methyl)oxetan-3-yl)phenyl)pyrazolo[1,5-a]pyrimidine-7-carboxamide

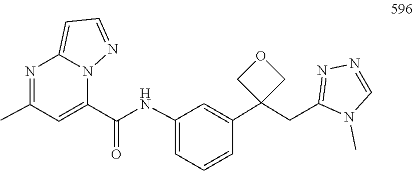 [SMILES: Cc1cc(C(=O)Nc2cccc(C3(Cc4nncn4C)COC3)c2)n2nccc2n1]

The amide bond formation reaction was carried out in a similar fashion as for Example 56 using 5-methylpyrazolo[1,5-a]pyrimidine-7-carboxylic acid (0.12 g, 0.65 mmol, 1.2 eq.) and Example R (0.13 g, 0.54 mmol, 1 eq.) as reactants afforded the title compound (60 mg, 28%) as an off-white solid: ¹H NMR (500 MHz, DMSO-d₆) 11.98 (s, 1H), 8.38 (d, J=2.4 Hz, 1H), 8.22 (s, 1H), 7.72 (dd, J=8.2, 2.0 Hz, 1H), 7.55 (s, 1H), 7.39-7.32 (m, 2H), 6.85-6.77 (m, 2H), 4.96 (d, J=6.0 Hz, 2H), 4.87 (d, J=6.0 Hz, 2H), 3.53 (s, 2H), 2.99 (s, 3H), 2.65 (s, 3H); LCMS: C₂₁H₂₁N₇O₂ requires: 403, found: m/z=404 [M+H]⁺.

### Example 597: 2,5-dimethyl-N-(3-(3-((4-methyl-4H-1,2,4-triazol-3-yl)methyl)oxetan-3-yl)phenyl)pyrazolo[1,5-a]pyrimidine-7-carboxamide

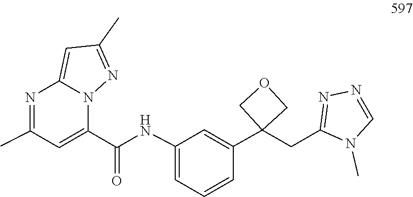 [SMILES: Cc1cc(C(=O)Nc2cccc(C3(Cc4nncn4C)COC3)c2)n2nc(C)cc2n1]

The amide bond formation reaction was carried out in a similar fashion as for Example 56 using 2,5-dimethylpyrazolo[1,5-a]pyrimidine-7-carboxylic acid (0.094 g, 0.49 mmol, 1.2 eq.) and Example R (0.10 g, 0.41 mmol, 1 eq.) as reactants afforded the title compound (22 mg, 13%) as an off-white solid: 1H NMR (500 MHz, acetonitrile-d₃) δ 12.76 (s, 1H), 7.92 (s, 1H), 7.76-7.70 (m, 1H), 7.66 (s, 1H), 7.41-7.34 (m, 2H), 6.81 (dt, J=7.9, 1.1 Hz, 1H), 6.57 (s, 1H), 5.04 (d, J=6.1 Hz, 2H), 4.99 (d, J=6.1 Hz, 2H), 3.55 (s, 2H), 2.92 (s, 3H), 2.66 (s, 3H), 2.62 (s, 3H); LCMS: C₂₁H₂₁N₇O₂ requires: 417, found: m/z=418 [M+H]⁺.

### Example 598: 5-Methyl-N-(3-(3-((4-methyl-4H-1,2,4-triazol-3-yl)methyl)oxetan-3-yl)phenyl)-2-(trifluoromethyl)pyrazolo[1,5-a]pyrimidine-7-carboxamide

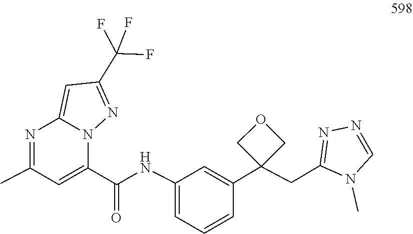 [SMILES: Cc1cc(C(=O)Nc2cccc(C3(Cc4nncn4C)COC3)c2)n2nc(C(F)(F)F)cc2n1]

The amide bond formation reaction was carried out in a similar fashion as for Example 56 using 5-methyl-2-(trifluoromethyl)pyrazolo[1,5-a]pyrimidine-7-carboxylic acid (0.12 g, 0.49 mmol, 1.2 eq.) and Example R (0.10 g, 0.41 mmol, 1 eq.) as reactants afforded the title compound (50 mg, 61%) as an off-white solid: 1H NMR (500 MHz, acetonitrile-d₃) δ 11.74 (s, 1H), 7.91 (d, J=9.4 Hz, 2H), 7.58 (ddd, J=8.1, 2.2, 1.0 Hz, 1H), 7.47 (t, J=2.0 Hz, 1H), 7.38 (t, J=7.9 Hz, 1H), 7.17 (s, 1H), 6.84 (dt, J=7.5, 1.3 Hz, 1H), 5.04 (d, J=6.0 Hz, 2H), 4.98 (d, J=6.0 Hz, 2H), 3.55 (s, 2H), 2.96 (s, 3H), 2.74 (s, 3H); LCMS: C₂₂H₂₀F₃N₇O₂ requires: 471, found: m/z=472 [M+H]⁺.

### Example 599: 3-methyl-N-(3-(3-((4-methyl-4H-1,2,4-triazol-3-yl)methyl)oxetan-3-yl)phenyl)-5-(trifluoromethyl)pyrazolo[1,5-a]pyrimidine-7-carboxamide

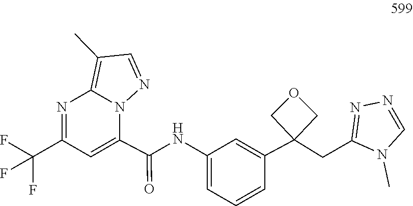 [SMILES: Cc1cnn2c(C(=O)Nc3cccc(C4(Cc5nncn5C)COC4)c3)cc(C(F)(F)F)nc12]

The amide bond formation reaction was carried out in a similar fashion as for Example 56 using 3-methyl-5-(trifluoromethyl)pyrazolo[1,5-a]pyrimidine-7-carboxylic acid (0.12 g, 0.49 mmol, 1.2 eq.) and Example R (0.10 g, 0.41 mmol, 1 eq.) as reactants afforded the title compound (64 mg, 33%) as an off-white solid: ¹H NMR (500 MHz, acetonitrile-d₃) δ 9.89 (s, 1H), 8.31 (s, 1H), 8.01 (s, 1H), 7.93 (s, 1H), 7.83 (ddd, J=8.1, 2.2, 1.0 Hz, 1H), 7.40 (t, J=2.0 Hz, 1H), 7.36 (t, J=7.9 Hz, 1H), 6.78 (dt, J=7.6, 1.3 Hz, 1H), 5.06-4.96 (m, 4H), 3.55 (s, 2H), 2.93 (s, 3H), 2.56 (s, 3H); LCMS: C₂₂H₂₀F₃N₇O₂ requires: 471, found: m/z=472 [M+H]⁺.

### Example 600: 2-(3-(3-((4-methyl-4H-1,2,4-triazol-3-yl)methyl)oxetan-3-yl)phenyl)-4-(trifluoromethyl)-6-(3-(trifluoromethyl)azetidin-1-yl)isoindolin-1-one

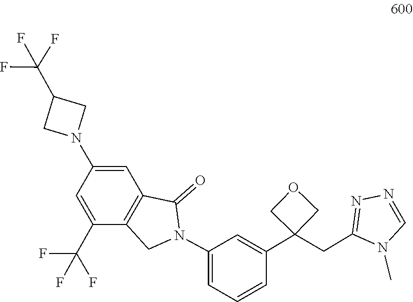 [SMILES: Cn1cnnc1CC1(c2cccc(N3Cc4c(cc(N5CC(C(F)(F)F)C5)cc4C(F)(F)F)C3=O)c2)COC1]

The C—N coupling reaction with potassium phosphate was carried out in a similar fashion as for Example 386 using 6-bromo-2-(3-{3-[(4-methyl-4H-1,2,4-triazol-3-yl)methyl]oxetan-3-yl}phenyl)-4-(trifluoromethyl)-2,3-dihydro-1H-isoindol-1-one (75 mg, 0.15 mmol, 1 eq.) and 3-(trifluoromethyl)azetidine hydrochloride (29 mg, 0.18 mmol, 1.2 eq.) as reactants afforded the title compound (53 mg, 65%) as a pink solid: 1H NMR (500 MHz, DMSO-d₆) δ 8.19 (s, 1H), 7.90 (dd, J=8.2, 2.1 Hz, 1H), 7.35 (s, 2H), 7.10-7.04 (m, 2H), 6.77 (d, J=7.5 Hz, 1H), 4.97 (d, J=6.3 Hz, 4H), 4.89 (d, J=6.1 Hz, 2H), 4.22 (t, J=8.5 Hz, 2H), 4.05 (dd, J=8.6, 5.2 Hz, 2H), 3.82-3.74 (m, 1H), 3.52 (s, 2H), 2.91 (s, 3H); LCMS: C₂₆H23F₆N₅O₂ requires: 551, found: m/z=552 [M+H]⁺.

### Example 601: 6-(3-(difluoromethyl)pyrrolidin-1-yl)-2-(3-(3-((4-methyl-4H-1,2,4-triazol-3-yl)methyl)oxetan-3-yl)phenyl)-4-(trifluoromethyl)isoindolin-1-one

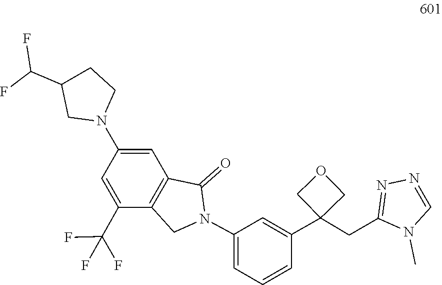 [SMILES: Cn1cnnc1CC1(c2cccc(N3Cc4c(cc(N5CCC(C(F)F)C5)cc4C(F)(F)F)C3=O)c2)COC1]

The C—N coupling reaction with potassium phosphate was carried out in a similar fashion as for Example 386 using 6-bromo-2-(3-{3-[(4-methyl-4H-1,2,4-triazol-3-yl)methyl]-oxetan-3-yl}phenyl)-4-(trifluoromethyl)-2,3-dihydro-1H-isoindol-1-one (75 mg, 0.15 mmol, 1 eq.) and 3-(difluoromethyl)pyrrolidine hydrochloride (23 mg, 0.15 mmol, 1.0 eq.) as reactants afforded the title compound (46 mg, 57%) as a pink solid: ¹H NMR (500 MHz, DMSO-d₆) δ 8.20 (s, 1H), 7.90 (dd, J=8.2, 2.1 Hz, 1H), 7.40-7.31 (m, 2H), 7.08 (q, J=2.4 Hz, 2H), 6.75 (d, J=8.1 Hz, 1H), 6.20 (dt, J=55, 5.0 Hz, 1H), 4.97 (d, J=6.1 Hz, 4H), 4.90 (d, J=6.0 Hz, 2H), 3.57 (t, J=9.1 Hz, 1H), 3.52 (s, 2H), 3.49 (dd, J=8.6, 5.1 Hz, 1H), 3.45-3.36 (m, 2H), 2.95 (dd, J=14.1, 6.9 Hz, 1H), 2.91 (s, 3H), 2.24-2.17 (m, 1H), 2.05 (dd, J=12.8, 7.4 Hz, 1H); LCMS: C₂₇H₂₆F₅N₅O₂ requires: 547, found: m/z=548 [M+H]⁺.

### Example 602: 6-(6,6-difluoro-2-azaspiro[3.3]heptan-2-yl)-2-(3-(3-((4-methyl-4H-1,2,4-triazol-3-yl)methyl)oxetan-3-yl)phenyl)-4-(trifluoromethyl)isoindolin-1-one

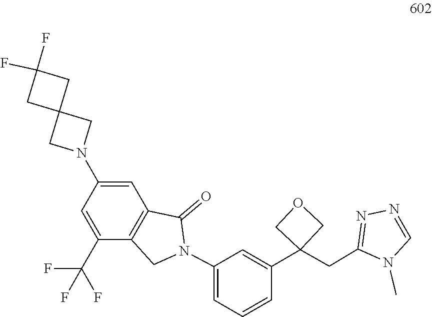 [SMILES: Cn1cnnc1CC1(c2cccc(N3Cc4c(cc(N5CC6(C5)CC(F)(F)C6)cc4C(F)(F)F)C3=O)c2)COC1]

The C—N coupling reaction with potassium phosphate was carried out in a similar fashion as for Example 386 using 6-bromo-2-(3-{3-[(4-methyl-4H-1,2,4-triazol-3-yl)methyl]oxetan-3-yl}phenyl)-4-(trifluoromethyl)-2,3-dihydro-1H-isoindol-1-one (75 mg, 0.15 mmol, 1 eq.) and 3-(difluoromethyl)pyrrolidine hydrochloride (30 mg, 0.18 mmol, 1.2 eq.) as reactants afforded the title compound (45 mg, 55%) as a pink solid: ¹H NMR (500 MHz, DMSO-d₆) δ 8.19 (s, 1H), 7.89 (dd, J=8.0, 2.1 Hz, 1H), 7.39-7.31 (m, 2H), 7.00-6.94 (m, 2H), 6.76 (d, J=7.6 Hz, 1H), 4.97 (d, J=5.9 Hz, 4H), 4.89 (d, J=6.1 Hz, 2H), 4.08 (s, 4H), 3.52 (s, 2H), 2.99-2.83 (m, 7H); LCMS: C₂₈H₂₆F₅N₅O₂ requires: 559, found: m/z=560 [M+H]⁺.

### Example 603: 6-(5,5-difluoro-2-azaspiro[3.3]heptan-2-yl)-2-(3-(3-((4-methyl-4H-1,2,4-triazol-3-yl)methyl)oxetan-3-yl)phenyl)-4-(trifluoromethyl)isoindolin-1-one

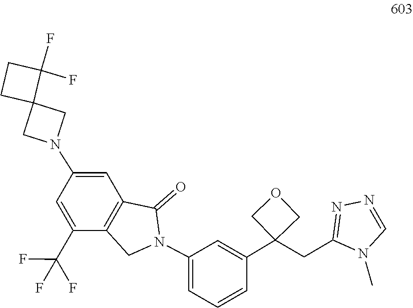 [SMILES: Cn1cnnc1CC1(c2cccc(N3Cc4c(cc(N5CC6(CCC6(F)F)C5)cc4C(F)(F)F)C3=O)c2)COC1]

The C—N coupling reaction with potassium phosphate was carried out in a similar fashion as for Example 386 using 6-bromo-2-(3-{3-[(4-methyl-4H-1,2,4-triazol-3-yl)methyl]oxetan-3-yl}phenyl)-4-(trifluoromethyl)-2,3-dihydro-1H-isoindol-1-one (75 mg, 0.15 mmol, 1 eq.) and 5,5-difluoro-2-azaspiro[3.3]heptane; trifluoroacetic acid (36 mg, 0.15 mmol, 1.0 eq.) as reactants afforded the title compound (30 mg, 36%) as a pink solid: ¹H NMR (500 MHz, DMSO-d₆) δ 8.19 (s, 1H), 7.89 (dd, J=8.4, 2.2 Hz, 1H), 7.38-7.31 (m, 2H), 7.05 (d, J=2.8 Hz, 2H), 6.76 (d, J=8.0 Hz, 1H), 5.05-4.94 (m, 4H), 4.89 (d, J=6.0 Hz, 2H), 4.24 (d, J=8.4 Hz, 2H), 3.92 (d, J=8.5 Hz, 2H), 3.52 (s, 2H), 2.91 (s, 3H), 2.61-2.52 (m, 2H), 2.09 (t, J=8.7 Hz, 2H); LCMS: C₂₈H₂₆F₅N₅O₂ requires: 559, found: m/z=560 [M+H]⁺.

### Example 604: 5-cyclopropyl-N-(3-(3-((4-methyl-4H-1,2,4-triazol-3-yl)methyl)oxetan-3-yl)phenyl)pyrazolo[1,5-a]pyrimidine-7-carboxamide

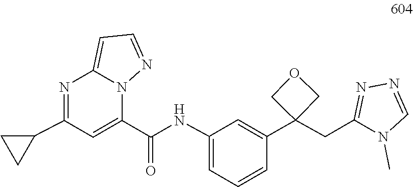 [SMILES: Cn1cnnc1CC1(c2cccc(NC(=O)c3cc(C4CC4)nc4ccnn34)c2)COC1]

The amide bond formation reaction was carried out in a similar fashion as for Example 56 using 5-cyclopropylpyrazolo[1,5-a]pyrimidine-7-carboxylic acid (0.10 g, 0.49 mmol, 1.2 eq.) and Example R (0.10 g, 0.41 mmol, 1 eq.) as reactants afforded the title compound (134 mg, 76%) as an off-white solid: ¹H NMR (500 MHz, acetonitrile-d₃) δ 12.69 (s, 1H), 8.28 (d, J=2.5 Hz, 1H), 7.92 (s, 1H), 7.79-7.73 (m, 2H), 7.41-7.33 (m, 2H), 6.80 (dt, J=7.7, 1.3 Hz, 1H), 6.68 (d, J=2.5 Hz, 1H), 5.04 (d, J=6.1 Hz, 2H), 4.99 (d, J=6.1 Hz, 2H), 3.55 (s, 2H), 2.93 (s, 3H), 2.34 (tt, J=7.6, 5.1 Hz, 1H), 1.19 (ddt, J=7.3, 5.2, 2.7 Hz, 4H); LCMS: C₂₃H₂₃N₇O₂ requires: 429, found: m/z=430 [M+H]⁺.

### Example 605: N-(3-(3-((4-methyl-4H-1,2,4-triazol-3-yl)methyl)oxetan-3-yl)phenyl)pyrazolo[1,5-a]pyrimidine-7-carboxamide

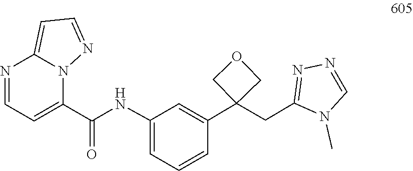 [SMILES: Cn1cnnc1CC1(c2cccc(NC(=O)c3ccnc4ccnn34)c2)COC1]

The amide bond formation reaction was carried out in a similar fashion as for Example 56 using pyrazolo[1,5-a]pyrimidine-7-carboxylic acid (0.050 g, 0.30 mmol, 1.2 eq.) and Example R (0.062 g, 0.25 mmol, 1 eq.) as reactants afforded the title compound (84 mg, 85%) as an off-white solid: ¹H NMR (500 MHz, acetonitrile-d₃) δ 12.64 (s, 1H), 8.76 (d, J=4.2 Hz, 1H), 8.40 (d, J=2.5 Hz, 1H), 7.92 (s, 1H), 7.85 (d, J=4.2 Hz, 1H), 7.78 (ddd, J=8.1, 2.1, 0.9 Hz, 1H), 7.42-7.32 (m, 2H), 6.96 (d, J=2.5 Hz, 1H), 6.81 (dt, J=7.7, 1.3 Hz, 1H), 5.04 (d, J=6.0 Hz, 2H), 5.00 (d, J=6.1 Hz, 2H), 3.55 (s, 2H), 2.94 (s, 3H); LCMS: C₂₀H₁₉N₇O₂ requires: 389, found: m/z=390 [M+H]⁺.

### Example 606: 3,5-dimethyl-N-(3-(3-((4-methyl-4H-1,2,4-triazol-3-yl)methyl)oxetan-3-yl)phenyl)pyrazolo[1,5-a]pyrimidine-7-carboxamide

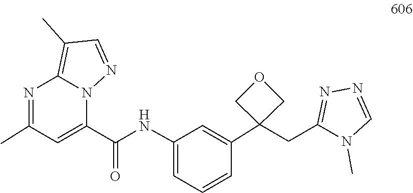 [SMILES: Cc1cc(C(=O)Nc2cccc(C3(Cc4nncn4C)COC3)c2)n2ncc(C)c2n1]

The amide bond formation reaction was carried out in a similar fashion as for Example 56 using 3,5-dimethylpyrazolo[1,5-a]pyrimidine-7-carboxylic acid (0.10 g, 0.49 mmol, 1.2 eq.) and Example R (0.10 g, 0.41 mmol, 1 eq.) as reactants afforded the title compound (156 mg, 91%) as an off-white solid: 1H NMR (500 MHz, acctonitrile-d₃) δ 9.96 (s, 1H), 8.17 (s, 1H), 8.06 (s, 1H), 7.83 (ddd, J=8.1, 2.2, 1.0 Hz, 1H), 7.62-7.56 (m, 1H), 7.42-7.32 (m, 2H), 6.75 (dt, J=7.6, 1.3 Hz, 1H), 5.03 (d, J=6.0 Hz, 2H), 4.99 (d, J=6.1 Hz, 2H), 3.59 (s, 2H), 2.94 (s, 3H), 2.83 (d, J=0.9 Hz, 3H), 2.51 (s, 3H); LCMS: C₂₂H₂₃N₇O₂ requires: 417, found: m/z=418 [M+H]⁺.

### Example 607: 2-(3-(3-((4-methyl-4H-1,2,4-triazol-3-yl)methyl)oxetan-3-yl)phenyl)-6-(3-methylazetidin-1-yl)-4-(trifluoromethyl)isoindolin-1-one

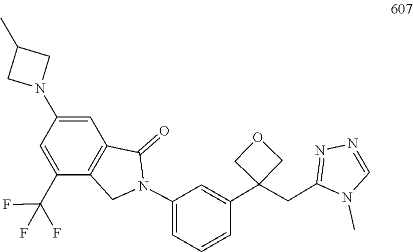 [SMILES: CC1CN(c2cc3c(c(C(F)(F)F)c2)CN(c2cccc(C4(Cc5nncn5C)COC4)c2)C3=O)C1]

The C—N coupling reaction with cesium carbonate was carried out in a similar fashion as for Example 386 using 6-bromo-2-(3-{3-[(4-methyl-1,2,4-triazol-3-yl)methyl]oxetan-3-yl}phenyl)-4-(trifluoromethyl)-3H-isoindol-1-one (50 mg, 0.099 mmol, 1 eq.) and 3-methylazetidine hydrochloride (13 mg, 0.12 mmol, 1.2 eq.) as reactants afforded the title compound (23 mg, 47%) as an off-white solid: ¹H NMR (500 MHz, DMSO-d₆) δ 8.19 (s, 1H), 7.89 (dd, J=8.2, 2.1 Hz, 1H), 7.38-7.31 (m, 2H), 6.95-6.89 (m, 2H), 6.76 (d, J=7.3 Hz, 1H), 5.03-4.93 (m, 4H), 4.89 (d, J=6.1 Hz, 2H), 4.11 (t, J=7.6 Hz, 2H), 3.61-3.47 (m, 4H), 2.91 (s, 3H), 2.88-2.80 (m, 1H), 1.27 (d, J=6.8 Hz, 3H); LCMS: C₂₆H₂₆F₃N₅O₂ requires: 497, found: m/z=498 [M+H]⁺

### Example 608: 6-(2,3-dimethylazetidin-1-yl)-2-(3-(3-((4-methyl-4H-1,2,4-triazol-3-yl)methyl)oxetan-3-yl)phenyl)-4-(trifluoromethyl)isoindolin-1-one

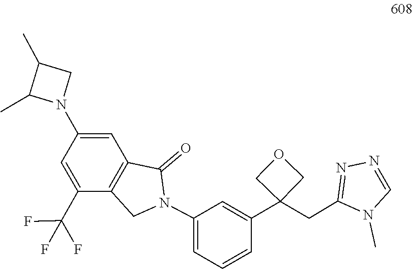 [SMILES: CC1CN(c2cc3c(c(C(F)(F)F)c2)CN(c2cccc(C4(Cc5nncn5C)COC4)c2)C3=O)C1C]

The C—N coupling reaction with cesium carbonate was carried out in a similar fashion as for Example 386 using 6-bromo-2-(3-{3-[(4-methyl-1,2,4-triazol-3-yl)methyl]oxetan-3-yl}phenyl)-4-(trifluoromethyl)-3H-isoindol-1-one (50 mg, 0.099 mmol, 1 eq.) and 2,3-dimethylazetidine hydrochloride (14 mg, 0.12 mmol, 1.2 eq.) as reactants afforded the title compound (26 mg, 51%, mixture of 3.6:1 diasteoreomers) as an off-white solid: ¹H NMR (major isomer, 500 MHz, DMSO-d₆) δ 8.19 (s, 1H), 7.91-7.85 (m, 1H), 7.40-7.31 (m, 2H), 6.99-6.89 (m, 2H), 6.76 (dd, J=7.8, 1.5 Hz, 1H), 5.04-4.92 (m, 4H), 4.89 (d, J=6.1 Hz, 2H), 4.19 (t, J=7.6 Hz, 1H), 3.79 (p, J=6.1 Hz, 1H), 3.52 (s, 2H), 3.26 (t, J=7.1 Hz, 1H), 2.91 (d, J=2.6 Hz, 3H), 2.45-2.39 (m, 1H), 1.45 (d, J=6.1 Hz, 3H), 1.18 (d, J=6.8 Hz, 3H); LCMS: C₂₇H₂₈F₃N₅O₂ requires: 511, found: m/z=512 [M+H]⁺.

### Example 609: 4-cyclopropyl-6-(3-(difluoromethyl)azetidin-1-yl)-2-(3-(3-((4-methyl-4H-1,2,4-triazol-3-yl)methyl)oxetan-3-yl)phenyl)-2,3-dihydro-1H-pyrrolo[3,4-c]pyridin-1-one

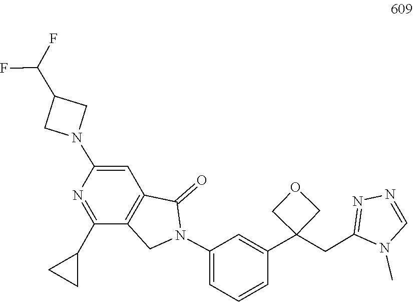 [SMILES: Cn1cnnc1CC1(c2cccc(N3Cc4c(cc(N5CC(C(F)F)C5)nc4C4CC4)C3=O)c2)COC1]

The C—N coupling reaction with cesium carbonate was carried out in a similar fashion as for Example 386 using 6-chloro-4-cyclopropyl-2-(3-{3-[(4-methyl-1,2,4-triazol-3-yl)methyl]oxetan-3-yl}phenyl)-3H-pyrrolo[3,4-c]pyridin-1-one (60 mg, 0.14 mmol, 1 eq.) and 3-(difluoromethyl)azetidine hydrochloride (40 mg, 0.28 mmol, 2 eq.) as reactants afforded the title compound (26 mg, 37%) as an off-white solid: ¹H NMR (500 MHz, DMSO-d₆) δ 8.21 (s, 1H), 7.96 (dd, J=8.1, 2.1 Hz, 1H), 7.40-7.32 (m, 2H), 6.80-6.75 (m, 1H), 6.47 (s, 1H), 6.51-6.24 (m, 1H), 5.05-4.94 (m, 4H), 4.90 (d, J=6.1 Hz, 2H), 4.12-4.01 (m, 2H), 3.89 (dd, J=8.6, 5.5 Hz, 2H), 3.51 (s, 2H), 3.18 (d, J=5.0 Hz, 1H), 2.92 (s, 3H), 2.11-2.05 (m, 1H), 1.09 (dt, J=5.2, 2.8 Hz, 2H), 1.02 (dt, J=8.3, 3.1 Hz, 2H); LCMS: C₂₇H₂₈F₂N₆O₂ requires: 506, found: m/z=507 [M+H]⁺.

### Example 610: 2-cyclopropyl-6-(3-(difluoromethyl)azetidin-1-yl)-N-(3-(3-((4-methyl-4H-1,2,4-triazol-3-yl)methyl)oxetan-3-yl)phenyl)pyrimidine-4-carboxamide

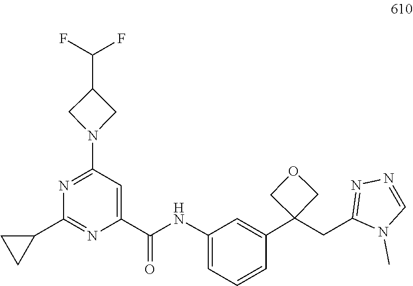 [SMILES: Cn1cnnc1CC1(c2cccc(NC(=O)c3cc(N4CC(C(F)F)C4)nc(C4CC4)n3)c2)COC1]

Step 1: Synthesis of ethyl 2-cyclopropyl-6-[3-(difluoromethyl)azetidin-1-yl]pyrimidine-4-carboxylate. The C—N coupling reaction with potassium phosphate was carried out in a similar fashion as for Example 386 using ethyl 6-chloro-2-cyclopropylpyrimidine-4-carboxylate (50 mg, 0.22 mmol, 1 eq.) and 3-(difluoromethyl)azetidine hydrochloride (38 mg, 0.26 mmol, 1.2 eq.) as reactants afforded the title compound (41 mg, 63%) as an off-white solid.

Step 2: Synthesis of 2-cyclopropyl-6-[3-(difluoromethyl)azetidin-1-yl]pyrimidine-4-carboxylic acid. A solution of ethyl 2-cyclopropyl-6-[3-(difluoromethyl)azetidin-1-yl]pyrimidine-4-carboxylate (41 mg, 0.14 mmol, 1 eq.) in THF (0.49 mL) was treated with an aqueous solution of lithium hydroxide (1M, 0.21 mL, 0.21 mmol, 1.5 eq.) at rt. The mixture was stirred for 72 h at rt and quenched with hydrochloric acid (2M, 0.11 mL, 0.21 mmol, 1.5 eq.). THF was removed and the mixture was dried on a lyophilizer to yield the title compound.

Step 3: 2-cyclopropyl-6-(3-(difluoromethyl)azetidin-1-yl)-N-(3-(3-((4-methyl-4H-1,2,4-triazol-3-yl)methyl)oxetan-3-yl)phenyl)pyrimidine-4-carboxamide. The amide bond formation reaction was carried out in a similar fashion as for Example 56 using the 2-cyclo-propyl-6-[3-(difluoromethyl)azetidin-1-yl]pyrimidine-4-carboxylic acid (0.14 mmol, 1 eq.) and Example R (40 mg, 0.17 mmol, 1.2 eq.) as reactants afforded the title compound (37 mg, 54%) as an off-white solid: ¹H NMR (500 MHz, DMSO-d₆) δ 10.24 (s, 1H), 8.21 (s, 1H), 7.79 (dd, J=8.1, 2.0 Hz, 1H), 7.45 (t, J=2.0 Hz, 1H), 7.28 (t, J=7.9 Hz, 1H), 6.81 (s, 1H), 6.71-6.65 (m, 1H), 6.54-6.27 (m, 1H), 4.94 (d, J=5.9 Hz, 2H), 4.86 (d, J=6.0 Hz, 2H), 4.21 (t, J=9.0 Hz, 2H), 4.04 (d, J=7.2 Hz, 2H), 3.49 (s, 2H), 3.30 (d, J=4.8 Hz, 1H), 2.93 (s, 3H), 2.18-2.11 (m, 1H), 1.09 (dt, J=5.9, 3.1 Hz, 2H), 0.99 (dq, J=10.1, 3.5 Hz, 2H); LCMS: C₂₅H₂₇F₂N₇O₂ requires: 495, found: m/z=496 [M+H]⁺.

### Example 611: 6-cyclobutyl-2-(3-(3-((4-methyl-4H-1,2,4-triazol-3-yl)methyl)oxetan-3-yl)phenyl)-4-(trifluoromethyl)isoindolin-1-one

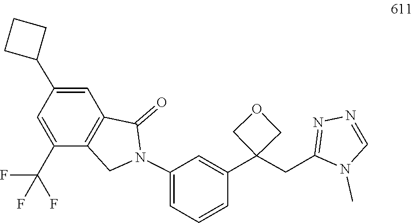 [SMILES: Cn1cnnc1CC1(c2cccc(N3Cc4c(cc(C5CCC5)cc4C(F)(F)F)C3=O)c2)COC1]

6-bromo-2-(3-(3-((4-methyl-4H-1,2,4-triazol-3-yl)methyl)oxetan-3-yl)phenyl)-4-(trifluoromethyl)isoindolin-1-one (50 mg, 0.10 mmol, 1 eq) was coupled with cyclobutyl zinc bromide as in Step 1 of Example 339 to afford 9.8 mg (21%) of the title compounds as an off-white solid. ¹H NMR (500 MHz, DMSO-d₆) δ 8.14-8.20 (s, 1H), 7.91 (dd, J=8.2, 1.9 Hz, 2H), 7.84 (s, 1H), 7.42-7.32 (m, 2H), 6.78 (d, J=13 Hz, 1H), 5.09 (s, 2H), 4.97 (d, J=6.0 Hz, 2H), 4.90 (d, J=6.1 Hz, 2H), 3.78 (p, J=8.8 Hz, 1H), 3.52 (s, 2H), 2.92 (s, 3H), 2.42-2.36 (m, 1H), 2.19 (pd, J=9.2, 2.7 Hz, 2H), 2.10-1.97 (m, 2H), 1.92-1.84 (m, 1H); LCMS: C₂₆H₂₅F₃N₄O₂ requires: 482, found: m/z=482 [M+H]⁺.

### Example 612: 6-(2,5-dihydrofuran-3-yl)-2-(3-(3-((4-methyl-4H-1,2,4-triazol-3-yl)methyl)oxetan-3-yl)phenyl)-4-(trifluoromethyl)isoindolin-1-one

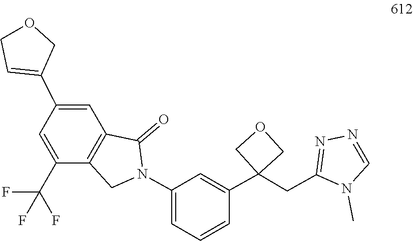 [SMILES: Cn1cnnc1CC1(c2cccc(N3Cc4c(cc(C5=CCOC5)cc4C(F)(F)F)C3=O)c2)COC1]

The Suzuki coupling reaction was carried out in a similar fashion as Example 67 using 6-bromo-2-(3-{3-[(4-methyl-4H-1,2,4-triazol-3-yl)methyl]oxetan-3-yl}phenyl)-4-(trifluoromethyl)-2,3-dihydro-1H-isoindol-1-one (65 mg, 0.13 mmol, 1 eq.) and 2-(2,5-dihydrofuran-3-yl)-4,4,5,5-tetramethyl-1,3,2-dioxaborolane (50 mg, 0.26 mmol, 2.0 eq.) as reactants afforded the title compound (58 mg, 92%) as a grey solid: 1H NMR (500 MHz, DMSO-dd) δ 8.20 (d, J=2.1 Hz, 1H), 8.08 (s, 1H), 8.02 (s, 1H), 7.91 (dd, J=8.1, 2.1 Hz, 1H), 7.41-7.33 (m, 2H), 6.95 (t, J=2.1 Hz, 1H), 6.79 (d, J=7.7 Hz, 1H), 5.14 (s, 2H), 5.03 (td, J=5.0, 2.1 Hz, 2H), 4.98 (d, J=5.9 Hz, 2H), 4.90 (d, J=6.0 Hz, 2H), 4.83-4.76 (m, 2H), 3.53 (s, 2H), 2.92 (s, 3H); LCMS: C₂₆H₂₃F₃N₄O₃ requires: 496, found: m/z=497 [M+H]⁺.

### Example 613: 2-(3-(3-((4-methyl-4H-1,2,4-triazol-3-yl)methyl)oxetan-3-yl)phenyl)-6-(THF-3-yl)-4-(trifluoromethyl)isoindolin-1-one

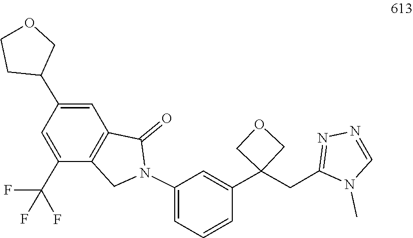 [SMILES: Cn1cnnc1CC1(c2cccc(N3Cc4c(cc(C5CCOC5)cc4C(F)(F)F)C3=O)c2)COC1]

The hydrogenation reaction was carried out in a similar fashion as Step 9 in Example B using 6-(2,5-dihydrofuran-3-yl)-2-(3-(3-((4-methyl-4H-1,2,4-triazol-3-yl)methyl)oxetan-3-yl)phenyl)-4-(trifluoromethyl)isoindolin-1-one (55 mg, 0.11 mmol) as reactant afforded the title compound (7.6 mg, 14%) as an off-white solid: ¹H NMR (500 MHz, DMSO-d₆) δ 8.19 (s, 1H), 7.99-7.87 (m, 3H), 7.41-7.33 (m, 2H), 6.78 (d, J=7.7 Hz, 1H), 5.09 (s, 2H), 4.97 (d, J=6.0 Hz, 2H), 4.90 (d, J=6.0 Hz, 2H), 4.10-4.05 (m, 1H), 4.02 (dt, J=8.4, 4.2 Hz, 1H), 3.84 (q, J=7.8 Hz, 1H), 3.68 (dd, J=6.7, 4.3 Hz, 1H), 3.52 (s, 2H), 2.92 (s, 3H), 2.42-2.40 (m, 1H), 2.05-1.96 (m, 2H); LCMS: C₂₆H₂₅F₃N₄O₃ requires: 497, found: m/z=498 [M+H]⁺.

### Example 614: 6-(2,5-dihydrofuran-3-yl)-2-(3-(3-((4-methyl-4H-1,2,4-triazol-3-yl)methyl)oxetan-3-yl)phenyl)-4-(trifluoromethyl)isoindolin-1-on

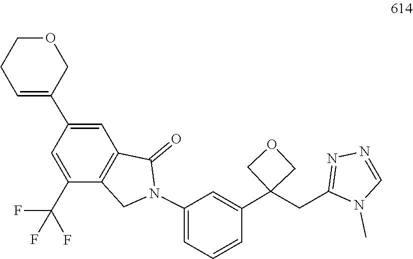 [SMILES: Cn1cnnc1CC1(c2cccc(N3Cc4c(cc(C5=CCCOC5)cc4C(F)(F)F)C3=O)c2)COC1]

The Suzuki coupling reaction was carried out in a similar fashion as Example 67 using 6-bromo-2-(3-{3-[(4-methyl-4H-1,2,4-triazol-3-yl)methyl]oxetan-3-yl}phenyl)-4-(trifluoromethyl)-2,3-dihydro-1H-isoindol-1-one (100 mg, 0.20 mmol, 1 eq.) and 2-(5,6-dihydro-2H-pyran-3-yl)-4,4,5,5-tetramethyl-1,3,2-dioxaborolane (62 mg, 0.30 mmol, 1.5 eq.) as reactants afforded the title compound (90 mg, 90%) as a grey solid: ¹H NMR (500 MHz, DMSO-d₆) δ 8.20 (s, 1H), 7.97 (d, J=10.2 Hz, 2H), 7.90 (dd, J=8.1, 2.2 Hz, 1H), 7.43-7.33 (m, 2H), 6.78 (d, J=7.4 Hz, 1H), 6.62 (td, J=4.2, 2.1 Hz, 1H), 5.12 (s, 2H), 4.97 (d, J=6.0 Hz, 2H), 4.90 (d, J=6.0 Hz, 2H), 4.55 (q, J=2.5 Hz, 2H), 3.78 (t, J=5.5 Hz, 2H), 3.52 (s, 2H), 2.92 (s, 3H), 2.32 (tt, J=5.3, 2.8 Hz, 2H); LCMS: C₂₇H₂₅F₃N₄O₃ requires: 510, found: m/z=511 [M+H]⁺.

### Example 615: 2-(3-(3-((4-methyl-4H-1,2,4-triazol-3-yl)methyl)oxetan-3-yl)phenyl)-6-(tetrahydro-2H-pyran-3-yl)-4-(trifluoromethyl)isoindolin-1-one

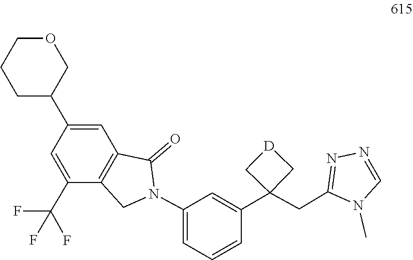

The hydrogenation reaction was carried out in a similar fashion as Step 9 in Example B using 6-(2,5-dihydrofuran-3-yl)-2-(3-(3-((4-methyl-4H-1,2,4-triazol-3-yl)methyl)oxetan-3-yl)phenyl)-4-(trifluoromethyl)isoindolin-1-one (90 mg, 0.18 mmol) as reactant afforded the title compound (24 mg, 26%) as an off-white solid: ¹H NMR (500 MHz, DMSO-d₆) δ 8.19 (d, J=2.2 Hz, 1H), 7.98 (d, J=12.6 Hz, 2H), 7.90 (dd, J=8.0, 2.1 Hz, 1H), 7.40-7.30 (m, 2H), 6.78 (d, J=7.8 Hz, 1H), 5.10 (d, J=17.8 Hz, 2H), 4.97 (d, J=6.0 Hz, 2H), 4.90 (d, J=6.1 Hz, 2H), 3.95-3.85 (m, 2H), 3.58-3.44 (m, 3H), 3.09 (d, J=11.1 Hz, 1H), 2.92 (d, J=2.1 Hz, 3H), 2.33 (s, 1H), 1.99 (s, 1H), 1.87 (d, J=9.5 Hz, 1H), 1.71-1.62 (m, 2H); LCMS: C₂₇H₂₇F₃N₄O₃ requires: 512, found: m/z=513 [M+H]⁺.

### Example 616: 6-(2-methyl-4,5-dihydrofuran-3-yl)-2-(3-(3-((4-methyl-4H-1,2,4-triazol-3-yl)methyl)oxetan-3-yl)phenyl)-4-(trifluoromethyl)isoindolin-1-one

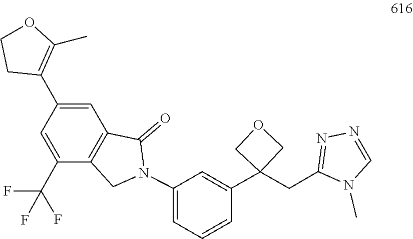 [SMILES: CC1=C(c2cc3c(c(C(F)(F)F)c2)CN(c2cccc(C4(Cc5nncn5C)COC4)c2)C3=O)CCO1]

The Suzuki coupling reaction was carried out in a similar fashion as Example 67 using 6-bromo-2-(3-{3-[(4-methyl-4H-1,2,4-triazol-3-yl)methyl]oxetan-3-yl}phenyl)-4-(trifluoromethyl)-2,3-dihydro-1H-isoindol-1-one (200 mg, 0.39 mmol, 1 eq.) and 2,3-dihydro-5-methylfuran-4-boronic acid pinacol ester (124 mg, 0.59 mmol, 1.5 eq.) as reactants afforded the title compound (47 mg, 23%) as a grey solid: ¹H NMR (500 MHz, DMSO-d₆) δ 8.20 (s, 1H), 7.89 (dd, J=8.0, 2.1 Hz, 1H), 7.82 (d, J=1.5 Hz, 1H), 7.76 (s, 1H), 7.42 (t, J=2.0 Hz, 1H), 7.36 (t, J=7.9 Hz, 1H), 6.77 (d, J=7.6 Hz, 1H), 5.10 (s, 2H), 4.97 (d, J=6.0 Hz, 2H), 4.90 (d, J=6.1 Hz, 2H), 4.40 (t, J=9.4 Hz, 2H), 3.52 (s, 2H), 3.12 (td, J=9.4, 2.0 Hz, 2H), 2.92 (s, 3H), 2.10 (d, J=1.7 Hz, 3H); LCMS: C₂₇H₂₅F₃N₄O₃ requires: 510, found: m/z=511 [M+H]⁺.

### Example 617: (R)-2-(3-(1,1-difluoro-3-(4-methyl-4H-1,2,4-triazol-3-yl)propan-2-yl)phenyl)-4-(trifluoromethyl)isoindolin-1-one

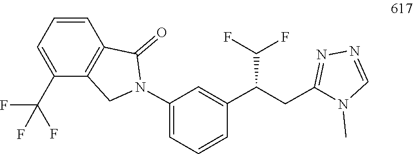 [SMILES: Cn1cnnc1C[C@H](c1cccc(N2Cc3c(cccc3C(F)(F)F)C2=O)c1)C(F)F]

Step 1: Synthesis of l-(3-aminophenyl)-2,2-difluoroethanone. To a solution of ethyl 2,2-difluoroacetate (6.0 g, 48 mmol) in THF (100 mL) was added 3-(chloromagnesio)-N,N-bis(trimethylsilyl)aniline (1 M in THF, 60.0 mL, 60 mmol) dropwise at −78° C. under nitrogen atmosphere. The resulting mixture was stirred at −78° C. for 4 h. The reaction was quenched by addition of saturated NH₄Cl aqueous solution and extracted with EtOAc. The combined organic layers were washed with brine, dried, and concentrated under vacuum. The residue was purified by chromatography A to afford the title compound (3.0 g, 36%) as a yellow oil.

Step 2: Synthesis of tert-butyl N-[3-(2,2-difluoroacetyl)phenyl]carbamate. A solution of l-(3-aminophenyl)-2,2-difluoroethanone (3.0 g, 18 mmol) and tert-butyl 2-methylpropan-1-ylium-2-yl dicarbonate (7.6 g, 35 mmol) in THF (50 mL) was stirred at 70° C. for 16 h. The solvent was removed under vacuum and the residue was purified by chromatography A to afford the title compound (3.6 g, 75%) as a yellow solid.

Step 3: Synthesis of ethyl (2Z)-3-[3-[(tert-butoxycarbonyl)amino]phenyl]-4,4-di-fluorobut-2-enoate. A solution of ethyl 2-(triphenyl-lambda5-phosphanylidene)acetate (6.0 g, 17 mmol) and tert-butyl N-[3-(2,2-difluoroacetyl)phenyl]carbamate (3.6 g, 13 mmol) in toluene (100 mL) was stirred at 110° C. for 16 h. The solvent was removed, and the residue was purified by chromatography A to afford the title compound (3.7 g, 81%) as a yellow oil.

Step 4: Synthesis of ethyl 3-[3-[(tert-butoxycarbonyl)amino]phenyl]-4,4-difluorobutanoate. To a solution of ethyl (2Z)-3-[3-[(tert-butoxycarbonyl)amino]phenyl]-4,4-difluorobut-2-enoate (3.7 g, 11 mmol) in ethanol (40 mL) was added Pd/C (300 mg) under nitrogen. The resulting mixture was stirred at rt for 4 h under H₂ (2 atm) atmosphere. The solids were filtered off and the filtrate was concentrated under vacuum to afford the title compound as a colorless syrup, which was used without purification.

Step 5: Synthesis of tert-butyl N-[3-[1,1-difluoro-3-(hydrazinecarbonyl)propan-2-yl]phenyl]carbamate. A solution of ethyl 3-[3-[(tert-butoxycarbonyl)amino]phenyl]-4,4-difluorobutanoate obtained in the previous step and N₂H₄-H₂O (10 mL) in ethanol (40 mL) was stirred at rt for 16 h. The solvent was removed to afford the title compound as a white solid, which was used without purification.

Step 6: Synthesis of tert-butyl N-(3-[1,1-difluoro-3-[(methylcarbamothioyl)-aminocarbamoyl] propan-2-yl]phenyl)carbamate. A solution of tert-butyl N-[3-[1,1-difluoro-3-(hydrazinecarbonyl)propan-2-yl]phenyl] carbamate obtained in the previous step and methyl isothiocyanate (830 mg, 11 mmol) in THF (100 mL) was stirred at rt for 16 h. The solvent was removed under vacuum to afford the title compound as a white solid, which was used without purification.

Step 7: Synthesis of tert-butyl N-[3-[1,1-difluoro-3-(4-methyl-5-sulfanyl-1,2,4-triazol-3-yl)propan-2-yl] phenyl]carbamate. A solution of tert-butyl N-(3-[1,1-difluoro-3-[(methylcarbamothioyl)aminocarbamoyl] propan-2-yl]phenyl)carbamate in NaOH (1 M, 50 mL, 50 mmol) was stirred at rt for 4 h. The mixture was acidified to pH 5 with HCl (3 N) and extracted with EtOAc. The combined organic layers were washed with brine, dried, and concentrated under vacuum. The residue was purified chromatography A to afford the title compound (2.3 g, 55% over four steps) as a white solid.

Step 8: Synthesis of tert-butyl N-[3-[1,1-difluoro-3-(4-methyl-1,2,4-triazol-3-yl)propan-2-yl] phenyl]carbamate. To a solution of tert-butyl N-[3-[1,1-difluoro-3-(4-methyl-5-sulfanyl-1,2,4-triazol-3-yl)propan-2-yl]phenyl]carbamate (2.3 g, 6.0 mmol) in DCM (30 mL) was added acetic acid (6.0 mL) and successively H₂O₂ (28% w/w, 4.5 g) dropwise at 0° C. The resulting mixture was stirred at rt for 1 h. The reaction was then quenched by saturated sodium bicarbonate aqueous solution and extracted with EtOAc. The combined organic layers were washed with brine, dried over Na₂SO₄ and concentrated under vacuum. The residue was purified chromatography A to afford the title compound (1.5 g, 71%) as a yellow solid.

Step 9: Synthesis of (S)-3-(1,1-difluoro-3-(4-methyl-4H-1,2,4-triazol-3-yl)propan-2-yl)aniline and (R)-3-(1,1-difluoro-3-(4-methyl-4H-1,2,4-triazol-3-yl)propan-2-yl)aniline. A solution of tert-butyl N-[3-[1,1-difluoro-3-(4-methyl-1,2,4-triazol-3-yl)propan-2-yl]phenyl]-carbamate (1.0 g, 2.8 mmol) in HCl (4 M in 1,4-dioxane, 10 mL, 40 mmol) was stirred at rt for 1 h. The solvent was removed under vacuum. The residue was purified by chromatography C to afford racemic 3-(1,1-difluoro-3-(4-methyl-4H-1,2,4-triazol-3-yl)propan-2-yl)aniline (940 mg) as a colorless semi-solid. The racemic product was separated by prep-chiral-SFC with CHIRALPAK IH along with CO₂ and isopropanol (2 mM NH₃-MeOH) to afford (S)-3-(1,1-difluoro-3-(4-methyl-4H-1,2,4-triazol-3-yl)propan-2-yl)aniline (354.5 mg) with shorter retention time on chiral-SFC as a yellow solid and (R)-3-(l, l-difluoro-3-(4-methyl-4H-1,2,4-triazol-3-yl)propan-2-yl)aniline (364.1 mg) with longer retention time on chiral-SFC as a yellow solid.

Step 10: Synthesis of (R)-2-(3-(1,1-difluoro-3-(4-methyl-4H-1,2,4-triazol-3-yl)propan-2-yl)phenyl)-4-(trifluoromethyl)isoindolin-1-one. The indolone formation reaction was carried out in a manner similar to EXAMPLE 260, step 2 using 3-[(2R)-1,1-difluoro-3-(4-methyl-1,2,4-triazol-3-yl)propan-2-yl]aniline (47 mg, 0.19 mmol, 1 eq.) and methyl 2-(bromomethyl)-3-(trifluoromethyl)benzoate (55 mg, 0.19 mmol, 1 eq.) as reactants to afford the title compound (14 mg, 17%) as an off-white solid: ¹H NMR (500 MHz, DMSO-d6) δ 8.30 (s, 1H), 8.08 (dd, J=24.0, 7.7 Hz, 2H), 7.97-7.86 (m, 2H), 7.81 (t, J=7.7 Hz, 1H), 7.42 (t, J=7.9 Hz, 1H), 7.22 (d, J=7.7 Hz, 1H), 6.45 (td, J=56.2, 4.1 Hz, 1H), 5.21 (s, 2H), 3.85 (s, 2H), 3.53 (s, 3H), 3.28 (dd, J=15.9, 9.0 Hz, 1H); LCMS: C₂₁H₁₇F₅N₄O requires: 436, found: m/z=437 [M+H]⁺.

### Example 618: (S)-6-((5-azaspiro[2.4]heptan-5-yl)methyl)-2-(3-(3-(fluoro(4-methyl-4H-1,2,4-triazol-3-yl)methyl)oxetan-3-yl)phenyl)-4-(trifluoromethyl)isoindolin-1-one

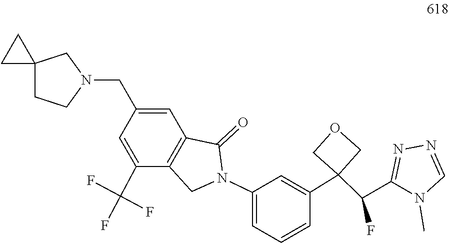 [SMILES: Cn1cnnc1[C@@H](F)C1(c2cccc(N3Cc4c(cc(CN5CCC6(CC6)C5)cc4C(F)(F)F)C3=O)c2)COC1]

The reductive amination reaction with sodium triacetoxyborohydride was carried out in a similar fashion as for EXAMPLE 447 using 2-(3-{3-[(S)-fluoro(4-methyl-1,2,4-triazol-3-yl)methyl]oxetan-3-yl}phenyl)-3-oxo-7-(trifluoromethyl)-1H-isoindole-5-carbaldehyde (100 mg, 0.21 mmol, 1 eq.) and 5-azaspiro[2.4]heptane hydrochloride (113 mg, 0.84 mmol, 4 eq.) as reactants afforded the title compound (2.9 mg, 2.5%) as an off-white solid: ¹H NMR (500 MHz, DMSO-d₆) δ 8.28 (s, 1H), 7.94-7.84 (m, 3H), 7.49 (t, J=1.9 Hz, 1H), 7.31 (t, J=8.0 Hz, 1H), 6.91 (d, J=8.0 Hz, 1H), 6.21 (d, J=45.5 Hz, 1H), 5.30 (d, J=6.6 Hz, 1H), 5.15 (d, J=6.1 Hz, 1H), 5.11-4.97 (m, 3H), 4.76 (dd, J=6.3, 4.0 Hz, 1H), 3.73 (s, 2H), 3.11 (s, 3H), 2.63 (t, J=6.8 Hz, 2H), 2.56-2.21 (m, 2H), 1.70 (t, J=6.8 Hz, 2H), 0.44 (dt, J=5.4, 1.8 Hz, 4H); LCMS: C₂₉H₂₉F₄N₅O₂ requires: 555, found: m/z=556 [M+H]⁺.

### Example 619: 6-((2-oxa-5-azabicyclo[2.2.1]heptan-5-yl)methyl)-2-(3-(3-((4-methyl-4H-1,2,4-triazol-3-yl)methyl)oxetan-3-yl)phenyl)-4-(trifluoromethyl)isoindolin-1-one

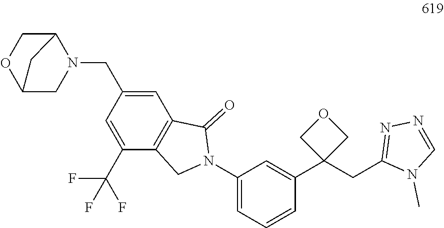 [SMILES: Cn1cnnc1CC1(c2cccc(N3Cc4c(cc(CN5CC6CC5CO6)cc4C(F)(F)F)C3=O)c2)COC1]

The reductive amination was carried out in a similar fashion as for 411, step 2 using Example Z (40 mg, 0.09 mmol, 1.0 eq.) and 2-oxa-5-azabicyclo[2.2.1]heptane (35 mg, 0.35 mmol) as reactants to afford the title compound (16 mg, 34%) as a white solid. ¹H NMR (500 MHz, DMSO-d₆) δ 8.20 (s, 1H), 8.03 (s, 1H), 7.98 (s, 1H), 7.89 (ddd, J=8.2, 2.3, 0.9 Hz, 1H), 7.40 (t, J=2.0 Hz, 1H), 7.36 (t, J=8.0 Hz, 1H), 6.77 (dt, J=7.7, 1.3 Hz, 1H), 5.10 (s, 2H), 4.94 (dd, J=38.5, 6.0 Hz, 4H), 4.12-3.72 (m, 4H), 3.57 (dd, J=7.6, 1.8 Hz, 1H), 3.52 (d, J=4.4 Hz, 2H), 2.91 (s, 2H), 2.76 (dd, J=9.9, 1.8 Hz, 1H), 2.47-2.43 (m, 1H), 1.87 (dd, J=9.7, 2.1 Hz, 1H), 1.64 (dt, J=9.8, 1.6 Hz, 1H); LCMS: C₂₈H₂₆F₃N₅O₃ requires: 539, found: m/z=540 [M+H]⁺.

### Example 620: 6-((4-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

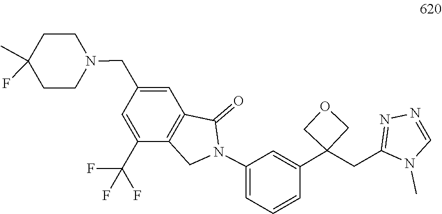 [SMILES: Cn1cnnc1CC1(c2cccc(N3Cc4c(cc(CN5CCC(C)(F)CC5)cc4C(F)(F)F)C3=O)c2)COC1]

The reductive amination was carried out in a similar fashion as for 411, step 2 using Example Z (136 mg, 0.30 mmol) and 4-fluoro-4-methylpiperidine hydrochloride (137 mg, 0.89 mmol) as reactants to afford the title compound (118 mg, 71%) as a white solid. ¹H NMR (500 MHz, Acetonitrile-d3) δ 8.43 (s, 1H), 8.06 (d, J=15.1 Hz, 2H), 7.81 (ddd, J=8.3, 2.2, 0.9 Hz, 1H), 7.33 (t, J=8.0 Hz, 1H), 7.29 (t, J=2.0 Hz, 1H), 6.78 (ddd, J=7.7, 1.9, 1.0 Hz, 1H), 4.98 (d, J=1.4 Hz, 2H), 4.90 (q, J=6.3 Hz, 4H), 4.33 (s, 2H), 3.66 (s, 2H), 3.28 (d, J=12.1 Hz, 2H), 3.08 (s, 2H), 2.94 (s, 3H), 2.04-1.91 (m, 4H), 1.32 (d, J=21.6 Hz, 3H); LCMS: C₂₉H₃₁F₄N₅O₂ requires: 557, found: m/z=558 [M+H]⁺.

### Example 621: 6-(((cis)-3-fluoro-4-methoxypiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

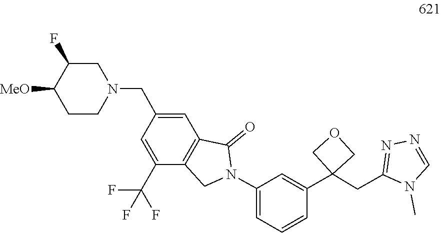 [SMILES: CO[C@@H]1CCN(Cc2cc3c(c(C(F)(F)F)c2)CN(c2cccc(C4(Cc5nncn5C)COC4)c2)C3=O)C[C@@H]1F]

The reductive amination was carried out in a similar fashion as for 411, step 2 using Example Z (50 mg, 0.11 mmol) and (cis)-3-fluoro-4-methoxypiperidine hydrochloride (19 mg, 0.11 mmol) as reactants to afford the title compound (12 mg, 19%) as a white solid. ¹H NMR (500 MHz, DMSO-d₆) δ 8.20 (s, 1H), 7.97 (d, J=24.9 Hz, 2H), 7.92-7.86 (m, 1H), 7.40 (t, J=1.9 Hz, 1H), 7.36 (t, J=8.0 Hz, 1H), 6.82-6.75 (m, 1H), 5.11 (s, 2H), 4.97 (d, J=6.0 Hz, 2H), 4.90 (d, J=6.1 Hz, 2H), 4.86 (s, 1H), 3.80-3.72 (m, 2H), 3.52 (s, 2H), 3.30 (s, 3H), 2.91 (s, 3H), 2.20 (d, J=37.2 Hz, 3H), 1.87-1.66 (m, 4H); LCMS: C₂₉H₃₁F₄N₅O₃ requires: 573, found: m/z=574 [M+H]⁺.

### Example 622a and 622b: (S)-6-(1-(4-fluoropiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one and (R)-6-(1-(4-fluoropiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one

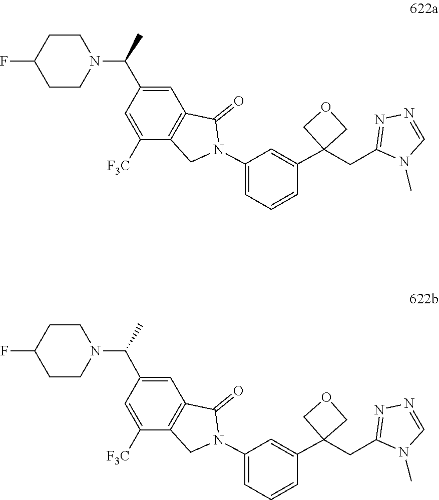 [SMILES: C[C@@H](c1cc2c(c(C(F)(F)F)c1)CN(c1cccc(C3(Cc4nncn4C)COC3)c1)C2=O)N1CCC(F)CC1 ; C[C@H](c1cc2c(c(C(F)(F)F)c1)CN(c1cccc(C3(Cc4nncn4C)COC3)c1)C2=O)N1CCC(F)CC1]

Step 1: Synthesis of 6-(1-(4-fluoropiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one. Amine displacement reaction was performed in a similar manner as (S)-6-(1-(4-fluoro-4-methyl-piperidin-1-yl)ethyl)-2-(3-(3-((4-methyl-4H-1,2,4-triazol-3-yl)methyl)oxetan-3-yl)phenyl)-4-(trifluoromethyl)isoindolin-1-one synthesis step-2 using 1-(2-(3-(3-((4-methyl-4H-1,2,4-triazol-3-yl)methyl)oxetan-3-yl)phenyl)-3-oxo-7-(trifluoromethyl)isoindolin-5-yl)ethyl methane-sulfonate (393 mg, 0.71 mmol) and 4-fluoropiperidine hydrochloride (196 mg, 1.4 mmol) to afford the title compound in 275 mg (69%) yield.

Step 2: Synthesis of (S)-6-(1-(4-fluoropiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one and (R)-6-(l-(4-fluoropiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one. 6-(1-(4-fluoropiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one (275 mg) was separated using column OZ with CO₂ and methanol as mobile phase to afford (S)-6-(1-(4-fluoropiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one (116 mg) and (R)-6-(1-(4-fluoropiperidin-1-yl)ethyl)-2-(3-(3-((4-methyl-4H-1,2,4-triazol-3-yl)methyl)oxetan-3-yl)phenyl)-4-(trifluoromethyl)isoindolin-1-one (109 mg).

¹H NMR (500 MHz, DMSO-d6) δ 8.18 (s, 1H), 7.98 (d, J=1.3 Hz, 1H), 7.93 (s, 1H), 7.88 (ddd, J=8.2, 2.2, 0.9 Hz, 1H), 7.39-7.32 (m, 2H), 6.80-6.74 (m, 1H), 5.13-5.06 (m, 2H), 4.96 (d, J=6.1 Hz, 2H), 4.89 (d, J=6.0 Hz, 2H), 4.72-4.53 (m, 1H), 3.83 (q, J=6.8 Hz, 1H), 3.51 (s, 2H), 2.89 (s, 3H), 2.35-2.23 (m, 1H), 1.94-1.57 (m, 3H), 1.37 (d, J=6.8 Hz, 3H); LCMS: C₂₉H₃₁F₄N₅O₂ requires: 557, found: m/z=558 [M+H]⁺.

¹H NMR (500 MHz, DMSO-d6) δ 8.18 (s, 1H), 7.98 (d, J=1.3 Hz, 1H), 7.93 (s, 1H), 7.88 (ddd, J=8.3, 2.2, 1.0 Hz, 1H), 7.40-7.32 (m, 2H), 6.77 (dt, J=7.9, 1.1 Hz, 1H), 5.09 (s, 2H), 4.96 (d, J=6.0 Hz, 2H), 4.89 (d, J=6.0 Hz, 2H), 4.75-4.52 (m, 1H), 3.83 (q, J=6.8 Hz, 1H), 3.51 (s, 2H), 2.89 (s, 3H), 2.35-2.27 (m, 1H), 1.89-1.69 (m, 2H), 1.37 (d, J=6.7 Hz, 3H); LCMS: C₂₉H₃₁F₄N₅O₂ requires: 557, found: m/z=558 [M+H]⁺.

### Example 623: (R)-6-((3-fluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

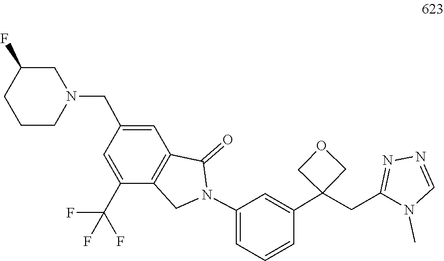 [SMILES: Cn1cnnc1CC1(c2cccc(N3Cc4c(cc(CN5CCC[C@@H](F)C5)cc4C(F)(F)F)C3=O)c2)COC1]

The reductive amination was carried out in a similar fashion as for 411, step 2 using Example Z (50 mg, 0.11 mmol) and (3R)-3-fluoropiperidine hydrochloride (46 mg, 0.33 mmol) as reactants to afford the title compound (28 mg, 12%) as a white solid. ¹H NMR (500 MHz, DMSO-d₆) δ 8.20 (s, 1H), 8.00 (s, 1H), 7.95 (s, 1H), 7.89 (ddd, J=8.3, 2.2, 1.0 Hz, 1H), 7.40 (t, J=2.0 Hz, 1H), 7.36 (t, J=7.9 Hz, 1H), 6.81-6.74 (m, 1H), 5.11 (s, 2H), 4.98 (d, J=6.0 Hz, 2H), 4.90 (d, J=6.0 Hz, 2H), 4.75-4.60 (m, 1H), 3.75 (s, 2H), 3.52 (s, 2H), 2.91 (s, 3H), 2.70 (dd, J=20.4, 11.4 Hz, 1H), 2.47-2.32 (m, 3H), 1.86-1.71 (m, 2H), 1.61-1.45 (m, 2H); LCMS: C₂₈H₂₉F₄N₅O₂ requires: 543, found: m/z=544 [M+H]⁺.

### Example 624: 6-((4-ethylpiperazin-1-yl)methyl)-2-(3-(3-((4-methyl-4H-1,2,4-triazol-3-yl)methyl)oxetan-3-yl)phenyl)-4-(trifluoromethyl)isoindolin-1-one

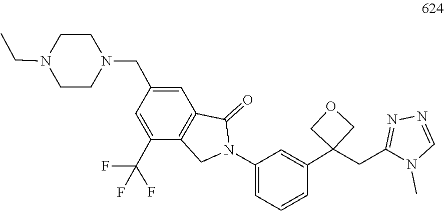 [SMILES: CCN1CCN(Cc2cc3c(c(C(F)(F)F)c2)CN(c2cccc(C4(Cc5nncn5C)COC4)c2)C3=O)CC1]

The reductive amination was carried out in a similar fashion as for 411, step 2 using Example Z (50 mg, 0.11 mmol) and (1-ethylpiperazine (38 mg, 0.33 mmol) as reactants to afford the title compound (15 mg, 25%) as a white solid. 1H NMR (500 MHz, DMSO-d₆) δ 8.19 (s, 1H), 7.99 (s, 1H), 7.94 (s, 1H), 7.89 (ddd, J=8.2, 2.2, 1.0 Hz, 1H), 7.40 (t, J=2.0 Hz, 1H), 7.36 (t, J=8.0 Hz, 1H), 6.78 (dt, J=7.8, 1.1 Hz, 1H), 5.11 (s, 2H), 4.97 (d, J=6.0 Hz, 2H), 4.90 (d, J=6.1 Hz, 2H), 3.69 (s, 2H), 3.52 (s, 2H), 2.91 (s, 3H), 2.38 (d, J=51.4 Hz, 10H), 0.99 (t, J=7.1 Hz, 3H); LCMS: C₂₉H₃₃F₃N₆O₂ requires: 554, found: m/z=555 [M+H]⁺.

### Example 625: 2-(3-(3-((4-methyl-4H-1,2,4-triazol-3-yl)methyl)oxetan-3-yl)phenyl)-6-((4-methylpiperazin-1-yl)methyl)-4-(trifluoromethyl)isoindolin-1-one

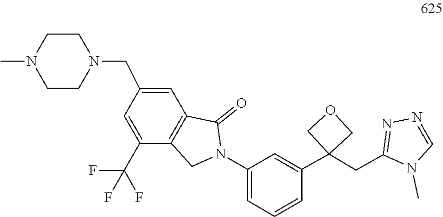 [SMILES: CN1CCN(Cc2cc3c(c(C(F)(F)F)c2)CN(c2cccc(C4(Cc5nncn5C)COC4)c2)C3=O)CC1]

The reductive amination was carried out in a similar fashion as for 411, step 2 using Example Z (50 mg, 0.11 mmol) and 1-methyl-piperazine (33 mg, 0.33 mmol) as reactants to afford the title compound (16 mg, 27%) as a white solid. ¹H NMR (500 MHz, DMSO-d₆) δ 8.20 (s, 1H), 7.99 (s, 1H), 7.93 (s, 1H), 7.92-7.87 (m, 1H), 7.40 (t, J=2.0 Hz, 1H), 7.36 (t, J=7.9 Hz, 1H), 6.78 (dt, J=7.6, 1.3 Hz, 1H), 5.11 (s, 2H), 4.97 (d, J=6.0 Hz, 2H), 4.90 (d, J=6.0 Hz, 2H), 3.69 (s, 2H), 3.52 (s, 2H), 2.91 (s, 3H), 2.42 (s, 8H), 2.17 (s, 3H); LCMS: C₂₈H₃₁F₃N₆O₂ requires: 540, found: m/z=541 [M+H]⁺.

### Example 626a and 626b: 6-(((3S,4S)-3,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one and 6-(((3R,4R)-3,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

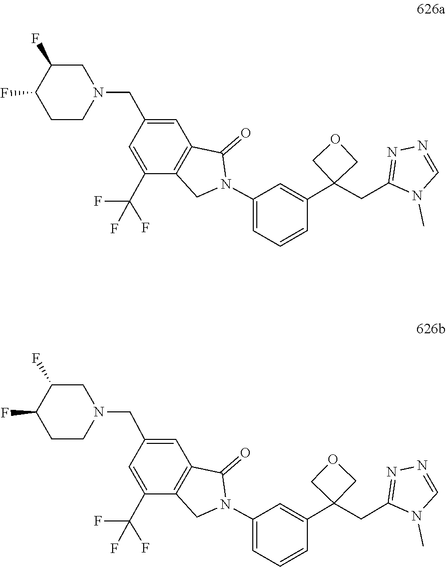 [SMILES: Cn1cnnc1CC1(c2cccc(N3Cc4c(cc(CN5CC[C@H](F)[C@@H](F)C5)cc4C(F)(F)F)C3=O)c2)COC1 ; Cn1cnnc1CC1(c2cccc(N3Cc4c(cc(CN5CC[C@@H](F)[C@H](F)C5)cc4C(F)(F)F)C3=O)c2)COC1]

Step 1: Synthesis of 6-(((trans)-3,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one. The reductive amination was carried out in a similar fashion as for 411, step 2 using Example Z (200 mg, 0.44 mmol) and (trans)-3,4-difluoropiperidine hydrochloride (207 mg, 1.31 mmol) as reactants to afford the title compound (8.9 mg, 3.6%) as a white solid.

Step 2: Separation of 6-(((3S,4S)-3,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one and 6-(((3R,4R)-3,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one. 6-(((trans)-3,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one was separated using IG column with CO₂ and methanol as mobile phase to afford the title compounds:6-(((3S,4S)-3,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one (2.6 mg): ¹H NMR (500 MHz, Acetonitrile-d₃) δ 7.92 (s, 1H), 7.89 (s, 1H), 7.83 (s, 1H), 7.79 (ddd, J=8.2, 2.2, 0.9 Hz, 1H), 7.28 (t, J=8.0 Hz, 1H), 7.22 (t, J=2.0 Hz, 1H), 6.66 (ddd, J=7.7, 1.7, 0.9 Hz, 1H), 4.94 (d, J=6.1 Hz, 2H), 4.93-4.88 (m, 4H), 4.82-4.71 (m, 1H), 4.67-4.57 (m, 1H), 3.67 (s, 2H), 3.47 (s, 2H), 2.76 (s, 3H), 2.48 (s, 1H), 2.37 (d, J=5.6 Hz, 1H), 2.02-1.93 (m, 2H), 1.83-1.71 (m, 2H); LCMS: C₂₈H₂₈F₅N₅O₂ requires: 561, found: m/z=562 [M+H]⁺.

6-(((3R,4R)-3,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one (4.1 mg): ¹H NMR (500 MHz, Acetonitrile-d₃) δ 7.92 (s, 1H), 7.89 (s, 1H), 7.83 (s, 1H), 7.79 (ddd, J=8.2, 2.2, 0.9 Hz, 1H), 7.28 (t, J=8.0 Hz, 1H), 7.22 (t, J=2.0 Hz, 1H), 6.66 (ddd, J=7.7, 1.7, 0.9 Hz, 1H), 4.94 (d, J=6.1 Hz, 2H), 4.92-4.87 (m, 4H), 4.81-4.70 (m, 1H), 4.66-4.56 (m, 1H), 3.67 (s, 2H), 3.47 (s, 2H), 2.76 (s, 3H), 2.48 (s, 1H), 2.37 (d, J=5.6 Hz, 1H), 2.02-1.93 (m, 2H), 1.83-1.71 (m, 2H); LCMS: C₂₈H₂₈F₅N₅O₂ requires: 561, found: m/z=562 [M+H]⁺.

### Example 627: 6-((3-fluoroazepan-1-yl)methyl)-2-(3-(3-((4-methyl-4H-1,2,4-triazol-3-yl)methyl)oxetan-3-yl)phenyl)-4-(trifluoromethyl)isoindolin-1-one

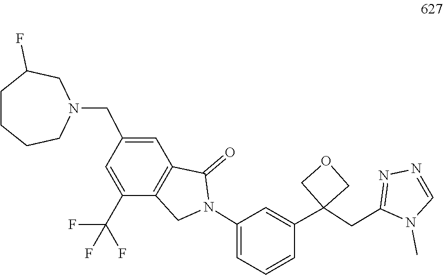 [SMILES: Cn1cnnc1CC1(c2cccc(N3Cc4c(cc(CN5CCCCC(F)C5)cc4C(F)(F)F)C3=O)c2)COC1]

The reductive amination was carried out in a similar fashion as for 411, step 2 using Example Z (50 mg, 0.11 mmol) and 3-fluoroazepane hydrochloride (51 mg, 0.33 mmol) as reactants to afford the title compound (7 mg, 11%) as a white solid. ¹H NMR (500 MHz, DMSO-dd) δ 8.19 (s, 1H), 8.03 (s, 1H), 8.00 (s, 1H), 7.88 (ddd, J=8.2, 2.2, 1.0 Hz, 1H), 7.41 (t, J=2.0 Hz, 1H), 7.36 (t, J=7.9 Hz, 1H), 6.77 (dt, J=8.0, 1.1 Hz, 1H), 5.11 (s, 2H), 4.97 (d, J=6.0 Hz, 2H), 4.90 (d, J=6.0 Hz, 2H), 4.72 (dd, J=46.9, 5.9 Hz, 1H), 3.97-3.86 (m, 2H), 3.52 (s, 2H), 2.97-2.83 (m, 3H), 2.65 (ddt, J=48.0, 12.9, 6.3 Hz, 3H), 2.01 (tt, J=10.2, 6.2 Hz, 1H), 1.90-1.77 (m, 1H), 1.77-1.55 (m, 3H), 1.51-1.39 (m, 2H); LCMS: C₂₉H₃₁F₄N₅O₂ requires: 557, found: m/z=558 [M+H]⁺.

### Example 628: 1-((2-(3-(3-((4-methyl-4H-1,2,4-triazol-3-yl)methyl)oxetan-3-yl)phenyl)-3-oxo-7-(trifluoromethyl)isoindolin-5-yl)methyl)piperidine-4-carbonitrile

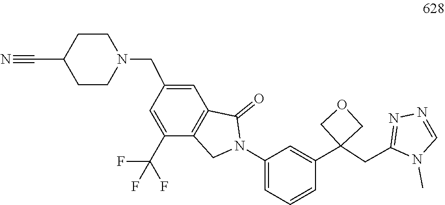 [SMILES: Cn1cnnc1CC1(c2cccc(N3Cc4c(cc(CN5CCC(C#N)CC5)cc4C(F)(F)F)C3=O)c2)COC1]

The reductive amination was carried out in a similar fashion as for 411, step 2 using Example Z (50 mg, 0.11 mmol) and piperidine-4-carbonitrile hydrochloride (48 mg, 0.33 mmol) as reactants to afford the title compound (13 mg, 21%) as a white solid. ¹H NMR (500 MHz, DMSO-d₆) δ 8.20 (s, 1H), 7.99 (s, 1H), 7.94 (s, 1H), 7.92-7.86 (m, 1H), 7.40 (t, J=2.0 Hz, 1H), 7.36 (t, J=8.0 Hz, 1H), 6.78 (d, J=7.8 Hz, 1H), 5.11 (s, 2H), 4.98 (d, J=6.0 Hz, 2H), 4.90 (d, J=6.1 Hz, 2H), 3.71 (s, 2H), 3.52 (s, 2H), 2.91 (s, 3H), 2.34 (s, 5H), 1.92-1.83 (m, 2H), 1.74 (dt, J=9.6, 5.1 Hz, 2H); LCMS: C₂₉H₂₉F₃N₆O₂ requires: 550, found: m/z=551 [M+H]⁺.

### Example 629: 6-((3-ethoxypiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

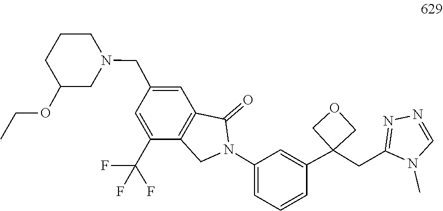 [SMILES: CCOC1CCCN(Cc2cc3c(c(C(F)(F)F)c2)CN(c2cccc(C4(Cc5nncn5C)COC4)c2)C3=O)C1]

The reductive amination was carried out in a similar fashion as for 411, step 2 using Example Z (50 mg, 0.11 mmol) and 3-ethoxypiperidine hydrochloride (54.4 mg, 0.33 mmol) as reactants to afford the title compound (5 mg, 9%) as a white solid. ¹H NMR (500 MHz, DMSO-d₆)δ 8.20 (s, 1H), 7.96 (d, J=22.2 Hz, 1H), 7.90 (d, J=8.0 Hz, 1H), 7.46-7.31 (m, 2H), 6.77 (d, J=7.6 Hz, 1H), 6.53 (d, J=4.0 Hz, 1H), 5.11 (s, 2H), 4.98 (d, J=5.9 Hz, 2H), 4.90 (d, J=6.0 Hz, 2H), 3.72 (s, 2H), 3.52 (s, 2H), 3.44 (dd, J=12.7, 7.0 Hz, 2H), 2.93 (d, J=18.2 Hz, 4H), 1.97 (d, J=47.5 Hz, 4H), 1.67 (s, 2H), 1.44 (s, 1H), 1.11 (dt, J=33.5, 6.8 Hz, 4H); LCMS: C₃₀H₃₄F₃N₅O₃ requires: 569, found: m/z=570 [M+H]⁺.

### Example 630 6-{[2-(difluoromethyl)morpholin-4-yl]methyl}-2-(3-{3-[(4-methyl-1,2,4-triazol-3-yl)methyl]oxetan-3-yl}phenyl)-4-(trifluoromethyl)-3H-isoindol-1-one

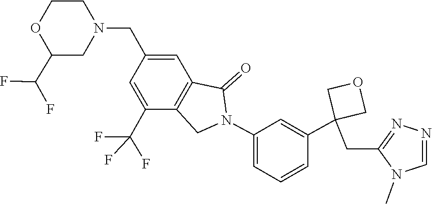 [SMILES: Cn1cnnc1CC1(c2cccc(N3Cc4c(cc(CN5CCOC(C(F)F)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)isoindoline-5-carbaldehyde (100 mg, 0.22 mmol) and 2-(difluoromethyl)morpholine hydrochloride (114 mg, 0.66 mmol) as reactants to afford the title compound (12 mg, 8%) as a white solid. ¹H NMR (500 MHz, Acetonitrile-d₃) δ 8.03 (s, 1H), 7.97-7.88 (m, 3H), 7.38 (dd, J=8.0 Hz, 1H), 7.34 (dd, J=2.0 Hz, 1H), 6.86-6.74 (m, 1H), 5.83 (td, J=55.2, 4.2 Hz, 1H), 5.05 (d, J=6.0 Hz, 2H), 5.03-4.95 (m, 4H), 3.98-3.89 (m, 1H), 3.75 (s, 2H), 3.68 (td, J=11.3, 2.6 Hz, 1H), 3.55 (s, 2H), 2.89 (s, 3H), 2.81 (d, J=11.2 Hz, 1H), 2.69 (d, J=11.8 Hz, 1H), 2.30 (td, J=11.3, 3.3 Hz, 1H), 2.24-2.19 (m, 1H), 2.14-2.11 (m, 1H); LCMS: C₂₈H28F₅N₅O₃ requires: 577, found: m/z=578 [M+H]⁺.

### Example 631: 6-((trans-3-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

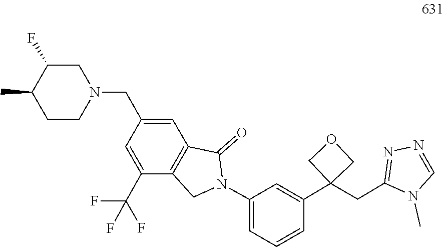 [SMILES: C[C@@H]1CCN(Cc2cc3c(c(C(F)(F)F)c2)CN(c2cccc(C4(Cc5nncn5C)COC4)c2)C3=O)C[C@H]1F]

The reductive amination was carried out in a similar fashion as for 411, step 2 using Example Z (50 mg, 0.11 mmol) and trans-3-fluoro-4-methylpiperidine hydrochloride (17 mg, 0.11 mmol) as reactants to afford the title compound (8 mg, 13%) as a white solid. ¹H NMR (500 MHz, DMSO-d₆) δ 8.20 (s, 1H), 8.00 (s, 1H), 7.94 (s, 1H), 7.92-7.87 (m, 1H), 7.40 (t, J=1.9 Hz, 1H), 7.36 (t, J=8.0 Hz, 1H), 6.78 (d, J=7.7 Hz, 1H), 5.11 (s, 2H), 4.98 (d, J=6.1 Hz, 2H), 4.90 (d, J=6.1 Hz, 2H), 4.20 (d, J=49.3 Hz, 1H), 3.77 (s, 2H), 3.52 (s, 2H), 3.09 (s, 1H), 2.91 (s, 3H), 2.71 (s, 1H), 2.06 (d, J=25.6 Hz, 2H), 1.70 (s, 1H), 1.52 (s, 1H), 1.25 (d, J=12.0 Hz, 1H), 1.01 (d, J=6.4 Hz, 3H); LCMS: C₂₉H₃₁F₄N₅O₂ requires: 557, found: m/z=558 [M+H]⁺.

### Example 632: 6-((4-methoxypiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

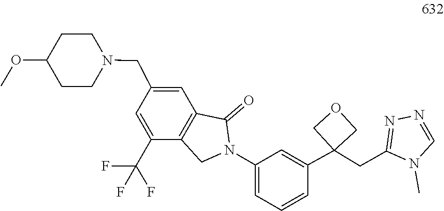 [SMILES: COC1CCN(Cc2cc3c(c(C(F)(F)F)c2)CN(c2cccc(C4(Cc5nncn5C)COC4)c2)C3=O)CC1]

The reductive amination was carried out in a similar fashion as for 411, step 2 using Example Z (50 mg, 0.11 mmol) and 4-methoxypiperidine hydrochloride (50 mg, 0.33 mmol) as reactants to afford the title compound (11 mg, 17%) as a white solid. ¹H NMR (500 MHz, DMSO-d₆) δ 8.20 (s, 1H), 7.98 (s, 1H), 7.93 (s, 1H), 7.89 (dd, J=8.1, 2.2 Hz, 1H), 7.41 (t, J=2.0 Hz, 1H), 7.36 (t, J=7.9 Hz, 1H), 6.77 (d, J=7.7 Hz, 1H), 5.10 (s, 2H), 4.98 (d, J=6.0 Hz, 2H), 4.90 (d, J=6.1 Hz, 2H), 3.68 (s, 2H), 3.52 (s, 2H), 3.20 (s, 3H), 3.21-3.16 (m, 1H) 2.91 (s, 3H), 2.66 (s, 2H), 2.17 (t, J=10.4 Hz, 2H), 1.88-1.79 (m, 2H), 1.51-1.40 (m, 2H); LCMS: C₂₉H32F3N5O3 requires: 556, found: m/z=557 [M+H]⁺.

### Example 633: 6-((4,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

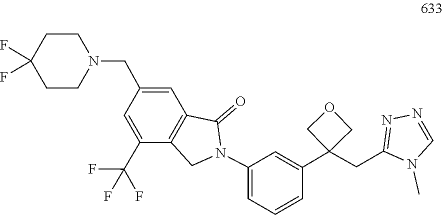 [SMILES: Cn1cnnc1CC1(c2cccc(N3Cc4c(cc(CN5CCC(F)(F)CC5)cc4C(F)(F)F)C3=O)c2)COC1]

The reductive amination was carried out in a similar fashion as for 411, step 2 using Example Z (50 mg, 0.11 mmol) and 4,4-difluoropiperidine hydrochloride (52 mg, 0.33 mmol) as reactants to afford the title compound (3 mg, 5%) as a white solid. ¹H NMR (500 MHz, DMSO-d₆) δ 8.20 (s, 1H), 8.03 (s, 1H), 7.98 (s, 1H), 7.89 (dd, J=8.1, 2.2 Hz, 1H), 7.41 (t, J=2.0 Hz, 1H), 7.36 (t, J=8.0 Hz, 1H), 6.79 (d, J=7.5 Hz, 1H), 5.12 (s, 2H), 4.98 (d, J=6.0 Hz, 2H), 4.90 (d, J=6.1 Hz, 2H), 3.80 (s, 2H), 3.52 (s, 2H), 2.92 (s, 3H), 2.55 (d, J=9.4 Hz, 4H), 2.07-1.92 (m, 4H); LCMS: C₂₈H28F5N5O2 requires: 561, found: m/z=562 [M+H]⁺.

### Example 634: 6-((cis-3,4-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

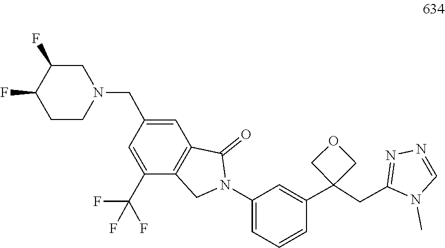 [SMILES: Cn1cnnc1CC1(c2cccc(N3Cc4c(cc(CN5CC[C@@H](F)[C@@H](F)C5)cc4C(F)(F)F)C3=O)c2)COC1]

The reductive amination was carried out in a similar fashion as for 411, step 2 using Example Z (50 mg, 0.11 mmol) and cis-3,4-difluoropiperidine hydrochloride (52 mg, 0.33 mmol) as reactants to afford the title compound (10 mg, 16%) as a white solid. ¹H NMR (500 MHz, Acetonitrile-d₃) δ 7.90 (d, J=1.3 Hz, 1H), 7.83-7.79 (m, 3H), 7.27 (t, J=8.0 Hz, 1H), 7.23 (t, J=2.0 Hz, 1H), 6.68 (ddd, J=7.7, 1.8, 1.0 Hz, 1H), 4.94 (d, J=6.1 Hz, 2H), 4.91-4.85 (m, 4H), 4.60-4.44 (m, 2H), 3.66 (s, 2H), 3.44 (s, 2H), 3.03-2.95 (m, 1H), 2.78 (s, 3H), 2.70-2.63 (m, 1H), 2.22 (ddd, J=11.2, 8.9, 6.4 Hz, 1H), 2.19-2.11 (m, 1H), 2.03-1.98 (m, 1H), 1.73-1.65 (m, 1H); LCMS: C₂₈H₂₈F₅N₅O₂ requires: 561, found: m/z=562 [M+H]⁺.

### Example 635: 6-((3,3-difluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

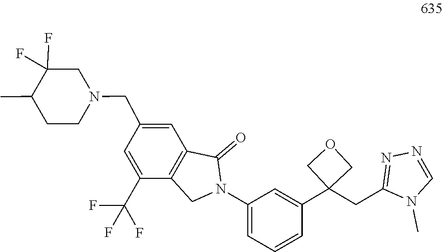 [SMILES: CC1CCN(Cc2cc3c(c(C(F)(F)F)c2)CN(c2cccc(C4(Cc5nncn5C)COC4)c2)C3=O)CC1(F)F]

The reductive amination was carried out in a similar fashion as for 411, step 2 using Example Z (50 mg, 0.11 mmol) and 3,3-difluoro-4-methylpiperidine hydrochloride (20 mg, 0.11 mmol) as reactants to afford the title compound (28 mg, 44%) as a white solid. 1H NMR (500 MHz, Acetonitrile-d₃) δ 7.88 (s, 1H), 7.84-7.76 (m, 3H), 7.26 (t, J=8.0 Hz, 1H), 7.21 (t, J=2.0 Hz, 1H), 6.67 (ddd, J=7.7, 1.8, 1.0 Hz, 1H), 4.93 (d, J=6.1 Hz, 2H), 4.88 (d, J=6.2 Hz, 4H), 3.66 (q, J=14.0 Hz, 2H), 3.43 (s, 2H), 2.91 (dddd, J=11.7, 9.9, 5.9, 1.6 Hz, 1H), 2.77 (s, 3H), 2.75-2.70 (m, 1H), 2.26 (ddd, J=28.1, 11.8, 2.3 Hz, 1H), 2.12 (tt, J=11.7, 2.2 Hz, 1H), 1.80-1.75 (m, 1H), 1.66-1.59 (m, 1H), 1.41 (qd, J=12.1, 4.1 Hz, 1H), 0.94 (d, J=6.7 Hz, 3H); LCMS: C₂₉H₃₀F₅N₅O₂ requires: 575, found: m/z=576 [M+H]⁺.

### Example 636: 6-((4,4-difluoro-6-azaspiro[2.5]octan-6-yl)methyl)-2-(3-(3-((4-methyl-4H-1,2,4-triazol-3-yl)methyl)oxetan-3-yl)phenyl)-4-(trifluoromethyl)isoindolin-1-one

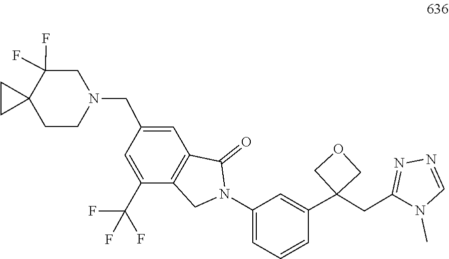 [SMILES: Cn1cnnc1CC1(c2cccc(N3Cc4c(cc(CN5CCC6(CC6)C(F)(F)C5)cc4C(F)(F)F)C3=O)c2)COC1]

The reductive amination was carried out in a similar fashion as for 411, step 2 using Example Z (50 mg, 0.11 mmol) and 4,4-difluoro-6-azaspiro[2.5]octane hydrochloride (60 mg, 0.33 mmol) as reactants to afford the title compound (27 mg, 43%) as a white solid. ¹H NMR (500 MHz, Acetonitrile-d₃) δ 7.91 (d, J=1.2 Hz, 1H), 7.87-7.75 (m, 3H), 7.27 (t, J=8.0 Hz, 1H), 7.23 (t, J=2.0 Hz, 1H), 6.68 (ddd, J=7.7, 1.7, 1.0 Hz, 1H), 4.94 (d, J=6.1 Hz, 2H), 4.92-4.83 (m, 4H), 3.72 (s, 2H), 3.44 (s, 2H), 2.78 (s, 3H), 2.71-2.63 (m, 2H), 2.52 (t, J=5.5 Hz, 2H), 1.55 (s, 2H), 0.79-0.72 (m, 2H), 0.41-0.33 (m, 2H); LCMS: C₃₀H₃₀F₅N₅O₂ requires: 587, found: m/z=588 [M+H]⁺.

### Example 637: 6-((3-methoxypiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

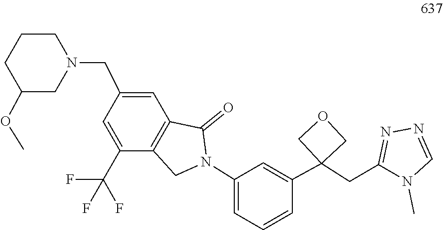 [SMILES: COC1CCCN(Cc2cc3c(c(C(F)(F)F)c2)CN(c2cccc(C4(Cc5nncn5C)COC4)c2)C3=O)C1]

The reductive amination was carried out in a similar fashion as for 411, step 2 using Example Z (25 mg, 0.05 mmol) and 3-methoxypiperidine hydrochloride (24 mg, 0.16 mmol) as reactants to afford the title compound (12 mg, 40%) as a white solid. ¹H NMR (500 MHz, Acetonitrile-d₃) δ 7.99 (s, 1H), 7.97-7.85 (m, 3H), 7.38 (t, J=8.0 Hz, 1H), 7.33 (t, J=2.0 Hz, 1H), 6.79 (ddd, J=7.7, 1.8, 1.0 Hz, 1H), 5.05 (d, J=6.1 Hz, 2H), 5.02-4.92 (m, 4H), 3.73-3.66 (m, 2H), 3.55 (s, 2H), 3.28 (s, 3H), 2.89 (s, 3H), 2.68-2.60 (m, 1H), 2.03 (t, J=9.7 Hz, 2H), 1.78-1.68 (m, 2H), 1.60-1.44 (m, 2H), 1.31-1.14 (m, 2H); LCMS: C₂₉H32F₃N₅O₃ requires: 555, found: m/z=556 [M+H]⁺.

### Example 638: 6-((3,4-dihydro-2,7-naphthyridin-2(1H)-yl)methyl)-2-(3-(3-((4-methyl-4H-1,2,4-triazol-3-yl)methyl)oxetan-3-yl)phenyl)-4-(trifluoromethyl)isoindolin-1-one

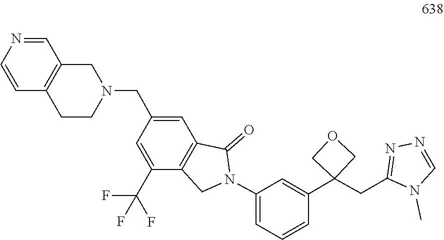 [SMILES: Cn1cnnc1CC1(c2cccc(N3Cc4c(cc(CN5CCc6ccncc6C5)cc4C(F)(F)F)C3=O)c2)COC1]

The reductive amination was carried out in a similar fashion as for 411, step 2 using Example Z (100 mg, 0.22 mmol) and 1,2,3,4-tetrahydro-2,6-naphthyridine (88 mg, 0.66 mmol) as reactants to afford the title compound (53 mg, 42%) as a white solid. ¹H NMR (500 MHz, Acetonitrile-d₃) δ 8.36 (s, 1H), 8.28 (d, J=5.0 Hz, 1H), 8.06 (s, 1H), 7.99 (s, 1H), 7.95-7.90 (m, 2H), 7.38 (dd, J=8.0 Hz, 1H), 7.34 (dd, J=2.0 Hz, 1H), 7.00 (d, J=5.2 Hz, 1H), 6.79 (dd, J=7.7, 1.8, 0.9 Hz, 1H), 5.05 (d, J=6.1 Hz, 2H), 5.00 (d, J=6.0 Hz, 4H), 3.89 (s, 2H), 3.67 (s, 2H), 3.55 (s, 2H), 2.92 (s, 2H), 2.89 (s, 3H), 2.84 (s, 2H); LCMS: C₃₁H₂₉F₃N₆O₃ requires: 575, found: m/z=576 [M+H]⁺.

### Example 639a and 639b: 6-(((3R,4R)-3-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one and 6-(((3S,4S)-3-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

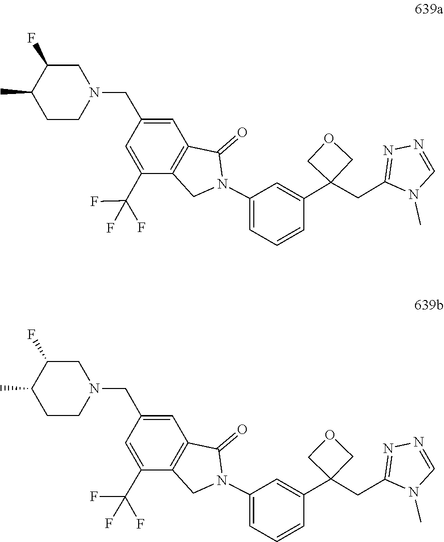 [SMILES: C[C@@H]1CCN(Cc2cc3c(c(C(F)(F)F)c2)CN(c2cccc(C4(Cc5nncn5C)COC4)c2)C3=O)C[C@@H]1F ; C[C@H]1CCN(Cc2cc3c(c(C(F)(F)F)c2)CN(c2cccc(C4(Cc5nncn5C)COC4)c2)C3=O)C[C@H]1F]

Step 1: Synthesis of 6-(cis-3-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one. The reductive animation was carried out in a similar fashion as for 411, step 2 using Example Z (50 mg, 0.11 mmol) and cis-3-fluoro-4-methylpiperidine hydrochloride (16 mg, 0.11 mmol) as reactants to afford the title compound (13 mg, 21%) as a white solid.

Step 2: 6-(((3R,4R)-3-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one and 6-(((3S,4S)-3-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one 6-(cis-3-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one was separated using IG column with CO₂ and methanol as mobile phase to afford the title compounds.

6-(((3R,4R)-3-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one (2.4 mg): ¹H NMR (500 MHz, Acetonitrile-d₃) δ 7.87 (s, 1H), 7.82-7.77 (m, 3H), 7.26 (t, J=8.0 Hz, 1H), 7.22 (t, J=2.0 Hz, 1H), 6.67 (ddd, J=7.6, 1.7, 0.9 Hz, 1H), 4.93 (d, J=6.1 Hz, 2H), 4.90-4.82 (m, 4H), 4.07 (dtd, J=49.1, 9.6, 4.7 Hz, 2H), 3.62 (s, 2H), 3.43 (s, 2H), 2.99 (dddd, J=10.6, 6.3, 4.6, 1.7 Hz, 1H), 2.77 (s, 3H), 2.67-2.59 (m, 1H), 2.02-1.92 (m, 1H), 1.63 (ddt, J=10.9, 6.7, 3.5 Hz, 1H), 1.46 (ddt, J=17.4, 9.4, 5.5 Hz, 1H), 1.25-1.15 (m, 1H), 0.95 (d, J=6.4 Hz, 3H); LCMS: C₂₉H₃₁F₄N₅O₂ requires: 557, found: m/z=558 [M+H]⁺.

6-(((3S,4S)-3-fluoro-4-methylpiperidin-1-yl)methyl)-2-(3-(3-((4-methyl-4H-1,2,4-triazol-3-yl)methyl)oxetan-3-yl)phenyl)-4-(trifluoromethyl)isoindolin-1-one (2.5 mg): ¹H NMR (500 MHz, Acetonitrile-d₃) δ 7.87 (s, 1H), 7.82-7.77 (m, 3H), 7.26 (t, J=8.0 Hz, 1H), 7.22 (t, J=2.0 Hz, 1H), 6.67 (ddd, J=7.6, 1.7, 0.9 Hz, 1H), 4.93 (d, J=6.1 Hz, 2H), 4.90-4.82 (m, 4H), 4.07 (dtd, J=49.1, 9.6, 4.7 Hz, 2H), 3.62 (s, 2H), 3.43 (s, 2H), 2.99 (dddd, J=10.6, 6.3, 4.6, 1.7 Hz, 1H), 2.77 (s, 3H), 2.67-2.58 (m, 1H), 2.00-1.92 (m, 1H), 1.63 (ddt, J=10.9, 6.7, 3.5 Hz, 1H), 1.46 (ddt, J=17.4, 9.4, 5.5 Hz, 1H), 1.24-1.14 (m, 1H), 0.95 (d, J=6.4 Hz, 3H); LCMS: C₂₉H₃₁F₄N₅O₂ requires: 557, found: m/z=558 [M+H]⁺.

### Example 640: 6-((6,6-difluoro-3-azabicyclo[3.1.1]heptan-3-yl)methyl)-2-(3-(3-((4-methyl-4H-1,2,4-triazol-3-yl)methyl)oxetan-3-yl)phenyl)-4-(trifluoromethyl)isoindolin-1-one

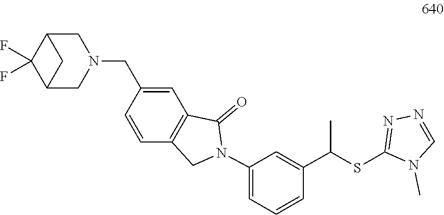 [SMILES: CC(Sc1nncn1C)c1cccc(N2Cc3ccc(CN4CC5CC(C4)C5(F)F)cc3C2=O)c1]

The reductive amination was carried out in a similar fashion as for 411, step 2 using Example Z (250 mg, 0.55 mmol) and 6,6-difluoro-3-azabicyclo[3.1.1]heptane hydrochloride (111 mg, 0.66 mmol) as reactants to afford the title compound (71 mg, 56%) as a white solid. ¹H NMR (500 MHz, Acetonitrile-d₃) δ 8.01 (s, 1H), 7.98-7.82 (m, 3H), 7.38 (dd, J=8.0 Hz, 1H), 7.34 (dd, J=2.0 Hz, 1H), 6.79 (d, J=7.6, 1.3 Hz, 1H), 5.05 (d, J=6.0 Hz, 2H), 5.04-4.93 (m, 4H), 3.87 (s, 2H), 3.55 (s, 2H), 3.23-3.14 (m, 2H), 2.80-2.69 (m, 2H), 1.85-1.70 (m, 2H); LCMS: C₂₉H₂₈F₅N₅O₂ requires: 573, found: m/z=574 [M+H]⁺.

### Example 641: 6-((6-azaspiro[2.5]octan-6-yl)methyl)-2-(3-(3-((4-methyl-4H-1,2,4-triazol-3-yl)methyl)oxetan-3-yl)phenyl)-4-(trifluoromethyl)isoindolin-1-one

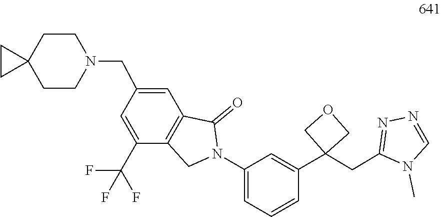 [SMILES: Cn1cnnc1CC1(c2cccc(N3Cc4c(cc(CN5CCC6(CC5)CC6)cc4C(F)(F)F)C3=O)c2)COC1]

The reductive amination was carried out in a similar fashion as for 411, step 2 using Example Z (150 mg, 0.33 mmol) and 6-azaspiro[2.5]octane hydrochloride (145 mg, 0.99 mmol) as reactants to afford the title compound (143 mg, 80%) as a white solid. ¹H NMR (500 MHz, Methanol-d₄) δ 8.17 (s, 1H), 8.12 (s, 1H), 8.03 (s, 1H), 7.76 (dd, J=8.1, 2.2 Hz, 1H), 7.47-7.35 (m, 2H), 6.77 (dt, J=7.6, 1.2 Hz, 1H), 5.12-5.00 (m, 6H), 4.02 (s, 1H), 3.65 (s, 2H), 2.90 (s, 3H), 2.81 (s, 4H), 1.53 (s, 4H), 0.37 (s, 4H); LCMS: C₃₀H₃₂F₃N₅O₂ requires: 551, found: m/z=552 [M+H]⁺.

### Example 642: 4-methyl-1-((2-(3-(3-((4-methyl-4H-1,2,4-triazol-3-yl)methyl)oxetan-3-yl)phenyl)-3-oxo-7-(trifluoromethyl)isoindolin-5-yl)methyl)piperidine-4-carbonitrile

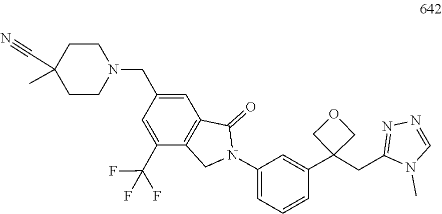 [SMILES: Cn1cnnc1CC1(c2cccc(N3Cc4c(cc(CN5CCC(C)(C#N)CC5)cc4C(F)(F)F)C3=O)c2)COC1]

The reductive amination was carried out in a similar fashion as for 411, step 2 using Example Z (150 mg, 0.33 mmol) and 4-methylpiperidine-4-carbonitrile hydrochloride (160 mg, 0.99 mmol) as reactants to afford the title compound (100 mg, 54%) as a white solid. ¹H NMR (500 MHz, Methanol-d₄) δ 8.17 (s, 1H), 8.06 (d, J=1.2 Hz, 1H), 7.95 (d, J=1.3 Hz, 1H), 7.76 (ddd, J=8.2, 2.3, 0.9 Hz, 1H), 7.43-7.35 (m, 2H), 6.74 (ddd, J=7.6, 1.7, 0.9 Hz, 1H), 5.10-5.03 (m, 6H), 3.76 (s, 2H), 3.65 (s, 2H), 2.89 (s, 5H), 2.38 (td, J=12.2, 2.4 Hz, 2H), 1.91 (ddd, J=13.9, 6.9, 4.0 Hz, 2H), 1.65 (ddd, J=13.6, 12.0, 3.9 Hz, 2H), 1.39 (s, 3H); LCMS: C₃₀H₃₁F₃N₆O₂ requires: 564, found: m/z=565 [M+H]⁺.

### Example 643: 6-((4-(difluoromethyl)piperidin-1-yl)methyl)-2-(3-(3-((4-methyl-4H-1,2,4-triazol-3-yl)methyl)oxetan-3-yl)phenyl)-4-(trifluoromethyl)isoindolin-1-one

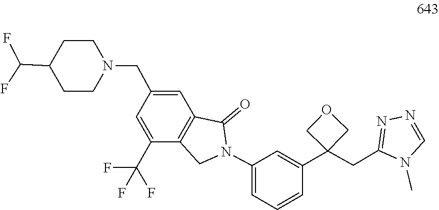 [SMILES: Cn1cnnc1CC1(c2cccc(N3Cc4c(cc(CN5CCC(C(F)F)CC5)cc4C(F)(F)F)C3=O)c2)COC1]

The reductive amination was carried out in a similar fashion as for 411, step 2 using Example Z (180 mg, 0.39 mmol) and 4-(difluoromethyl)piperidine hydrochloride (68 mg, 0.39 mmol) as reactants to afford the title compound (117 mg, 52%) as a white solid. ¹H NMR (500 MHz, Methanol-d₄) δ 8.18 (d, J=6.0 Hz, 1H), 8.17 (s, 1H), 8.10 (s, 1H), 7.76 (ddd, J=8.2, 2.3, 1.0 Hz, 1H), 7.45-7.35 (m, 2H), 6.81 (dt, J=8.0, 1.1 Hz, 1H), 5.95-5.68 (m, 1H), 5.23-5.10 (m, 2H), 5.07 (dd, J=6.2, 6.2 Hz, 4H), 4.36 (s, 2H), 3.45 (s, 2H), 2.91 (s, 3H), 2.18-2.00 (m, 5H), 1.75-1.56 (m, 4H); LCMS: C₂₉H₃₀F₃N₅O₂ requires: 575, found: m/z=576 [M+H]⁺.

### Example 644a and EXAMPLE 644b: (S)-6-((4,4-difluoro-3-(hydroxymethyl)piperidin-1-yl)methyl)-2-(3-(3-((4-methyl-4H-1,2,4-triazol-3-yl)methyl)oxetan-3-yl)phenyl)-4-(trifluoro-methyl)isoindolin-1-one and (R)-6-((4,4-difluoro-3-(hydroxymethyl)piperidin-1-yl)methyl)-2-(3-(3-((4-methyl-4H-1,2,4-triazol-3-yl)methyl)oxetan-3-yl)phenyl)-4-(trifluoromethyl)-isoindolin-1-one

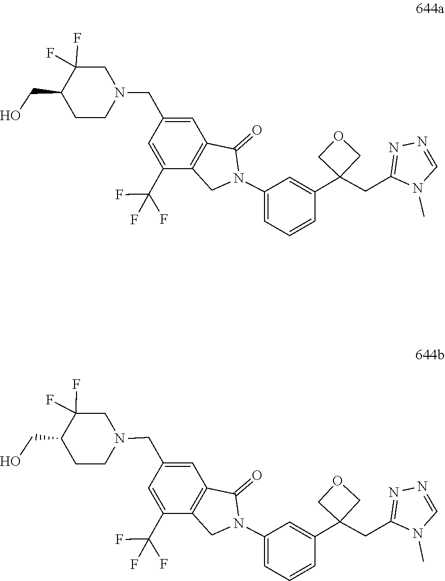 [SMILES: Cn1cnnc1CC1(c2cccc(N3Cc4c(cc(CN5CC[C@@H](CO)C(F)(F)C5)cc4C(F)(F)F)C3=O)c2)COC1 ; Cn1cnnc1CC1(c2cccc(N3Cc4c(cc(CN5CC[C@H](CO)C(F)(F)C5)cc4C(F)(F)F)C3=O)c2)COC1]

Step 1: 6-((3,3-difluoro-4-(hydroxymethyl)piperidin-1-yl)methyl)-2-(3-(3-((4-methyl-4H-1,2,4-triazol-3-yl)methyl)oxetan-3-yl)phenyl)-4-(trifluoromethyl)isoindolin-1-one. The reductive amination was carried out in a similar fashion as for 411, step 2 using Example Z (150 mg, 0.33 mmol) and (4,4-difluoropiperidin-3-yl)methanol hydrochloride (185 mg, 0.99 mmol) as reactants to afford the title compound (150 mg, 77%) as a white solid.

Step 2: (R)-6-((3,3-difluoro-4-(hydroxymethyl)piperidin-1-yl)methyl)-2-(3-(3-((4-methyl-4H-1,2,4-triazol-3-yl)methyl)oxetan-3-yl)phenyl)-4-(trifluoromethyl)isoindolin-1-one and (S)-6-((3,3-difluoro-4-(hydroxymethyl)piperidin-1-yl)methyl)-2-(3-(3-((4-methyl-4H-1,2,4-triazol-3-yl)methyl)oxetan-3-yl)phenyl)-4-(trifluoromethyl)isoindolin-1-one 6-((4,4-difluoro-3-(hydroxymethyl)piperidin-1-yl)methyl)-2-(3-(3-((4-methyl-4H-1,2,4-triazol-3-yl)methyl)oxetan-3-yl)phenyl)-4-(trifluoromethyl)isoindolin-1-one was separated using IG column with CO₂ and methanol as mobile phase to afford the title compounds.

(S)-6-((4,4-difluoro-3-(hydroxymethyl)piperidin-1-yl)methyl)-2-(3-(3-((4-methyl-4H-1,2,4-triazol-3-yl)methyl)oxetan-3-yl)phenyl)-4-(trifluoromethyl)isoindolin-1-one (25 mg) ¹H NMR (500 MHz, Methanol-d₄) δ 8.18 (s, 1H), 8.06 (s, 1H), 7.97 (s, 1H), 7.75 (ddd, J=8.2, 2.2, 0.9 Hz, 1H), 7.44-7.36 (m, 2H), 6.74 (ddd, J=7.7, 1.8, 1.0 Hz, 1H), 5.10-5.04 (m, 6H), 3.93 (dd, J=11.2, 4.3 Hz, 1H), 3.86-3.73 (m, 2H), 3.65 (s, 2H), 3.50 (dd, J=11.1, 8.0 Hz, 1H), 2.95 (dd, J=22.7, 10.3 Hz, 3H), 2.89 (s, 3H), 2.37 (ddd, J=37.9, 12.1, 2.3 Hz, 1H), 2.25 (t, J=11.6 Hz, 1H), 2.02-1.93 (m, 3H), 1.66-1.56 (m, 1H); LCMS: C₂₉H₃₀F₅N₅O₃ requires: 591, found: m/z=592 [M+H]⁺.

(R)-6-((4,4-difluoro-3-(hydroxymethyl)piperidin-1-yl)methyl)-2-(3-(3-((4-methyl-4H-1,2,4-triazol-3-yl)methyl)oxetan-3-yl)phenyl)-4-(trifluoromethyl)isoindolin-1-one (23 mg) ¹H NMR (500 MHz, Methanol-d₄) δ 8.18 (s, 1H), 8.06 (s, 1H), 7.97 (s, 1H), 7.75 (ddd, J=8.2, 2.2, 0.9 Hz, 1H), 7.43-7.36 (m, 2H), 6.74 (ddd, J=7.7, 1.8, 1.0 Hz, 1H), 5.10-5.04 (m, 6H), 3.93 (dd, J=11.2, 4.3 Hz, 1H), 3.87-3.73 (m, 2H), 3.65 (s, 2H), 3.50 (dd, J=11.1, 8.0 Hz, 1H), 2.95 (dd, J=22.7, 10.3 Hz, 3H), 2.89 (s, 3H), 2.37 (ddd, J=27.9, 12.1, 2.3 Hz, 1H), 2.25 (t, J=11.6 Hz, 1H), 2.02-1.91 (m, 3H), 1.66-1.53 (m, 1H); LCMS: C₂₉H₃₀F₅N₅O₃ requires: 591, found: m/z=592 [M+H]⁺.

### Example 645: 6-((5,8-dihydro-1,7-naphthyridin-7(6H)-yl)methyl)-2-(3-(3-((4-methyl-4H-1,2,4-triazol-3-yl)methyl)oxetan-3-yl)phenyl)-4-(trifluoromethyl)isoindolin-1-one

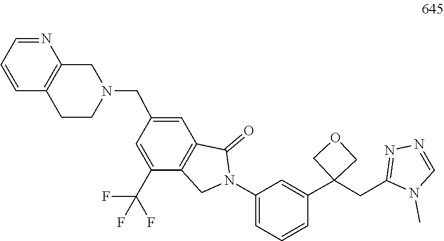 [SMILES: Cn1cnnc1CC1(c2cccc(N3Cc4c(cc(CN5CCc6cccnc6C5)cc4C(F)(F)F)C3=O)c2)COC1]

The reductive amination was carried out in a similar fashion as for 411, step 2 using Example Z (100 mg, 0.22 mmol) and 1,2,3,4-tetrahydro-2,6-naphthyridine (88 mg, 0.66 mmol) as reactants to afford the title compound (23 mg, 18%) as a white solid. ¹H NMR (500 MHz, Acetonitrile-d₃) δ 8.36-8.31 (m, 1H), 8.07 (s, 1H), 8.00 (s, 1H), 7.95-7.90 (m, 2H), 7.52 (dd, J=7.7, 1.6 Hz, 1H), 7.38 (dd, J=8.0 Hz, 1H), 7.34 (dd, J=2.0 Hz, 1H), 7.15 (dd, J=1.1, 4.7 Hz, 1H), 6.83-6.75 (m, 1H), 5.05 (d, J=6.1 Hz, 2H), 5.03-4.93 (m, 4H), 3.92 (s, 2H), 3.70 (s, 2H), 2.91 (t, J=5.8 Hz, 2H), 2.89 (s, 2H), 2.81 (t, J=5.8 Hz, 2H); LCMS: C₃₁H₂₉F₃N₆O₃ requires: 575, found: m/z=576 [M+H]⁺.

### Example 646: 6-((3-azabicyclo[4.1.0]heptan-3-yl)methyl)-2-(3-(3-((4-methyl-4H-1,2,4-triazol-3-yl)methyl)oxetan-3-yl)phenyl)-4-(trifluoromethyl)isoindolin-1-one

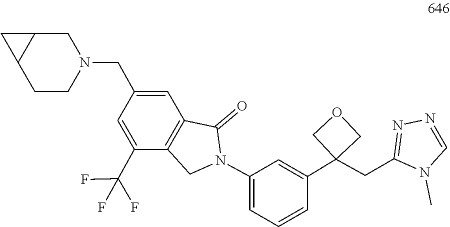 [SMILES: Cn1cnnc1CC1(c2cccc(N3Cc4c(cc(CN5CCC6CC6C5)cc4C(F)(F)F)C3=O)c2)COC1]

The reductive amination was carried out in a similar fashion as for 411, step 2 using Example Z (250 mg, 0.55 mmol) and 3-azabicyclo[4.1.0]heptane hydrochloride (220 mg, 1.64 mmol) as reactants to afford the title compound (71 mg, 24%) as a white solid. ¹H NMR (500 MHz, DMSO-d6) δ 8.20 (s, 1H), 7.96 (s, 1H), 7.93-7.85 (m, 2H), 7.42 (t, J=2.0 Hz, 1H), 7.36 (t, J=7.9 Hz, 1H), 6.77 (dt, J=7.8, 1.2 Hz, 1H), 5.10 (s, 2H), 4.98 (d, J=6.0 Hz, 2H), 4.90 (d, J=6.0 Hz, 2H), 3.33 (s, 2H), 2.91 (s, 3H), 2.72 (d, J=11.1 Hz, 1H), 2.57 (dt, J=10.7, 5.4 Hz, 2H), 2.27 (dt, J=11.0, 5.3 Hz, 1H), 2.04 (ddd, J=11.3, 9.2, 5.3 Hz, 1H), 1.94 (ddt, J=13.2, 9.9, 4.9 Hz, 1H), 1.68 (ddd, J=14.4, 8.4, 5.8 Hz, 1H), 1.08-0.98 (m, 1H), 0.98-0.88 (m, 1H), 0.56 (td, J=8.7, 3.7 Hz, 1H), 0.33 (td, J=5.2, 3.7 Hz, 1H); LCMS: C₂₉H₃₀F₃N₅O₂ requires: 536, found: m/z=537 [M+H]⁺.

### Example 647: 6-((4,4-difluoro-3-hydroxypiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

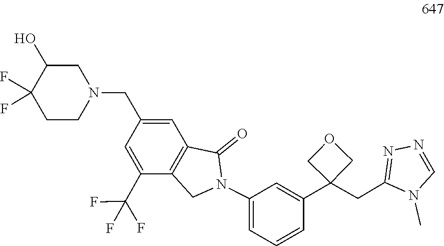 [SMILES: Cn1cnnc1CC1(c2cccc(N3Cc4c(cc(CN5CCC(F)(F)C(O)C5)cc4C(F)(F)F)C3=O)c2)COC1]

The reductive amination was carried out in a similar fashion as for 411, step 2 using Example Z (100 mg, 0.22 mmol) and 4,4-difluoropiperidin-3-ol hydrochloride (38 mg, 0.22 mmol) as reactants to afford the title compound (59 mg, 47%) as a white solid. ¹H NMR (500 MHz, Acetonitrile-d₃) δ 8.04 (s, 1H), 7.95 (s, 1H), 7.94-7.88 (m, 2H), 7.38 (t, J=8.0 Hz, 1H), 7.34 (dd, J=2.0 Hz, 1H), 6.79 (d, J=7.8, 1.1 Hz, 1H), 5.05 (d, J=6.0 Hz, 2H), 5.03-4.92 (m, 4H), 3.77 (s, 2H), 3.55 (s, 2H), 3.54-3.49 (m, 1H), 2.77-2.68 (m, 1H), 2.59-2.53 (m, 2H), 1.95 (d, J=5.5 Hz, 4H); LCMS: C₂₈H₂₈F₅N₅O₃ requires: 577, found: m/z=578 [M+H]⁺.

### Example 648: 6-((3,4-dihydro-2,6-naphthyridin-2(1H)-yl)methyl)-2-(3-(3-((4-methyl-4H-1,2,4-triazol-3-yl)methyl)oxetan-3-yl)phenyl)-4-(trifluoromethyl)isoindolin-1-one

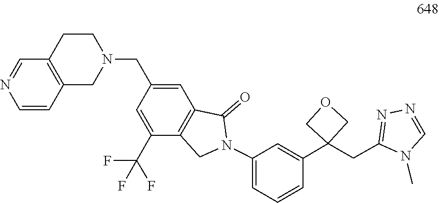 [SMILES: Cn1cnnc1CC1(c2cccc(N3Cc4c(cc(CN5CCc6cnccc6C5)cc4C(F)(F)F)C3=O)c2)COC1]

The reductive amination was carried out in a similar fashion as for 411, step 2 using Example Z (100 mg, 0.22 mmol) and 1,2,3,4-tetrahydro-2,7-naphthyridine hydrochloride (112 mg, 0.66 mmol) as reactants to afford the title compound (67 mg, 53%) as a white solid. ¹H NMR (500 MHz, Acetonitrile-d₃) δ 8.30 (d, J=5.0 Hz, 1H), 8.24 (s, 1H), 8.06 (s, 1H), 7.99 (s, 1H), 7.96-7.88 (m, 2H), 7.38 (dd, J=8.0 Hz, 1H), 7.34 (dd, J=2.0 Hz, 1H), 7.12 (d, J=5.0 Hz, 1H), 6.79 (dt, J=7.8, 1.1 Hz, 1H), 5.05 (d, J=6.1 Hz, 2H), 4.99 (d, J=6.1 Hz, 4H), 3.90 (s, 2H), 3.68 (s, 2H), 2.89 (s, 3H), 2.81 (t, J=5.9 Hz, 2H); LCMS: C₃₁H₂₉F₃N₆O₃ requires: 574, found: m/z=575 [M+H]⁺.

### Example 649: 2-(3-(3-((4-methyl-4H-1,2,4-triazol-3-yl)methyl)oxetan-3-yl)phenyl)-6-((2-methyl-6,7-dihydrooxazolo[4,5-c]pyridin-5(4H)-yl)methyl)-4-(trifluoromethyl)isoindolin-1-one

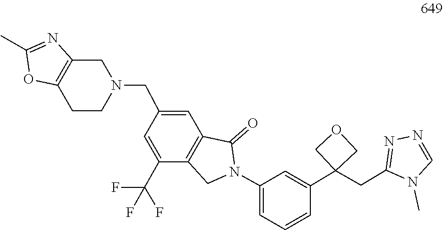 [SMILES: Cc1nc2c(o1)CCN(Cc1cc3c(c(C(F)(F)F)c1)CN(c1cccc(C4(Cc5nncn5C)COC4)c1)C3=O)C2]

The reductive amination was carried out in a similar fashion as for 411, step 2 using Example Z (200 mg, 0.44 mmol) and 2-methyl-4H,5H,6H,7H-[1,3]oxazolo[4,5-c]pyridine hydrochloride (114 mg, 0.66 mmol) as reactants to afford the title compound (205 mg, 80%) as a white solid. ¹H NMR (500 MHz, DMSO-d₆) δ 8.20 (s, 1H), 8.00 (s, 1H), 7.95 (s, 1H), 7.92-7.87 (m, 1H), 7.45-7.40 (m, 1H), 7.36 (dd, J=7.9 Hz, 1H), 6.78 (d, J=7.9, 1.2 Hz, 1H), 5.11 (s, 2H), 4.98 (d, J=6.1 Hz, 2H), 4.90 (d, J=6.1 Hz, 2H), 3.80-3.72 (m, 1H), 3.69 (d, J=2.4 Hz, 2H), 3.58-3.48 (m, 4H), 2.91 (s, 3H), 2.75-2.67 (m, 1H), 2.67-2.59 (m, 1H), 2.16-2.07 (m, 1H), 1.81 (t, J=11.2, 9.8 Hz, 1H), 1.04 (d, J=6.3 Hz, 3H); LCMS: C₃₀H₂₉F₃N₆O₃ requires: 578, found: m/z=579 [M+H]⁺.

### Example 650: 6-((3,4-dihydroisoquinolin-2(1H)-yl)methyl)-2-(3-(3-((4-methyl-4H-1,2,4-triazol-3-yl)methyl)oxetan-3-yl)phenyl)-4-(trifluoromethyl)isoindolin-1-one

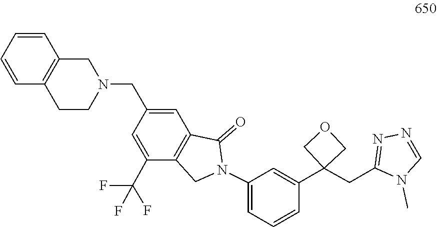 [SMILES: Cn1cnnc1CC1(c2cccc(N3Cc4c(cc(CN5CCc6ccccc6C5)cc4C(F)(F)F)C3=O)c2)COC1]

The reductive amination was carried out in a similar fashion as for 411, step 2 using Example Z (200 mg, 0.44 mmol) and tetrahydroisoquinoline (58 mg, 0.44 mmol) as reactants to afford the title compound (202 mg, 80%) as a white solid. ¹H NMR (500 MHz, DMSO-d₆) δ 8.20 (s, 1H), 8.06 (s, 1H), 8.02 (s, 1H), 7.90 (dd, J=8.1, 2.2 Hz, 1H), 7.41 (dd, J=2.0 Hz, 1H), 7.36 (dd, J=7.9 Hz, 1H), 7.18-7.09 (m, 3H), 7.09-7.00 (m, 1H), 6.77 (dt, J=7.8, 1.1 Hz, 1H), 5.12 (s, 2H), 4.98 (d, J=6.1 Hz, 2H), 4.90 (d, J=6.1 Hz, 2H), 3.89 (s, 2H), 3.61 (s, 2H), 3.52 (s, 2H), 2.91 (s, 3H), 2.85 (t, J=5.9 Hz, 2H), 2.75 (t, J=5.9 Hz, 2H); LCMS: C₃₀H₃₂F₃N₅O₃ requires: 573, found: m/z=574 [M+H]⁺.

### Example 651 and 652: 6-((l-methyl-1,4,5,6-tetrahydro-7H-pyrazolo[3,4-b]pyridin-7-yl)methyl)-2-(3-(3-((4-methyl-4H-1,2,4-triazol-3-yl)methyl)oxetan-3-yl)phenyl)-4-(trifluoromethyl)isoindolin-1-one and 6-((2-methyl-2,4,5,6-tetrahydro-7H-pyrazolo[3,4-b]pyridin-7-yl)methyl)-2-(3-(3-((4-methyl-4H-1,2,4-triazol-3-yl)methyl)oxetan-3-yl)phenyl)-4-(trifluoromethyl)isoindolin-1-one

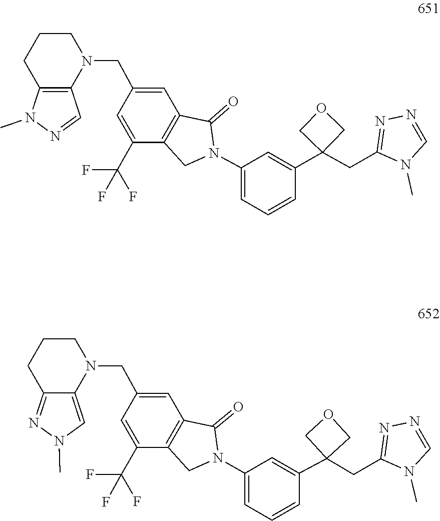 [SMILES: Cn1cnnc1CC1(c2cccc(N3Cc4c(cc(CN5CCCc6c5cnn6C)cc4C(F)(F)F)C3=O)c2)COC1 ; Cn1cc2c(n1)CCCN2Cc1cc2c(c(C(F)(F)F)c1)CN(c1cccc(C3(Cc4nncn4C)COC3)c1)C2=O]

The reductive amination was carried out in a similar fashion as for 411, step 2 using Example Z (250 mg, 0.55 mmol) and a mixture of l-methyl-4,5,6,7-tetrahydro-1H-pyrazolo[4,3-b]pyridine and 2-methyl-4,5,6,7-tetrahydro-2H-pyrazolo[4,3-b]pyridine (285 mg, 1.64 mmol) as reactants to afford the title compounds. 6-((l-methyl-1,5,6,7-tetrahydro-4H-pyrazolo[4,3-b]pyridin-4-yl)methyl)-2-(3-(3-((4-methyl-4H-1,2,4-triazol-3-yl)methyl)oxetan-3-yl)phenyl)-4-(trifluoromethyl)isoindolin-1-one (38 mg, 12%) was isolated as a white solid. ¹H NMR (500 MHz, Acetonitrile-d₃) δ 8.03 (s, 1H), 7.97 (s, 1H), 7.94-7.88 (m, 2H), 7.38 (dd, J=8.0 Hz, 1H), 7.33 (dd, J=2.0 Hz, 1H), 6.82 (s, 1H), 6.79 (ddd, J=7.7, 1.8, 1.0 Hz, 1H), 5.04 (d, J=6.1 Hz, 2H), 4.99 (d, J=6.0 Hz, 4H), 4.24 (s, 2H), 3.65 (s, 3H), 3.55 (s, 2H), 2.88 (d, J=3.0 Hz, 3H), 2.66 (d, J=13.1 Hz, 2H), 2.05-1.98 (m, 4H); LCMS: C₃₀H₃₀F₃N₇O₂ requires: 578, found: m/z=579 [M+H]⁺. 6-((2-methyl-2,5,6,7-tetrahydro-4H-pyrazolo[4,3-b]pyridin-4-yl)methyl)-2-(3-(3-((4-methyl-4H-1,2,4-triazol-3-yl)methyl)oxetan-3-yl)phenyl)-4-(trifluoromethyl)isoindolin-1-one (48 mg, 24%) was isolated as a white solid. ¹H NMR (500 MHz, Acetonitrile-d₃) δ 8.39 (s, 1H), 8.03 (s, 1H), 7.97 (s, 1H), 7.92 (ddd, J=8.3, 2.3, 1.0 Hz, 1H), 7.42 (t, J=8.0 Hz, 1H), 7.39 (t, J=2.0 Hz, 1H), 6.85 (ddd, J=7.6, 1.8, 0.9 Hz, 1H), 6.76 (s, 1H), 5.02 (d, J=9.7 Hz, 5H), 4.26 (s, 2H), 3.71 (s, 2H), 3.68 (s, 2H), 2.67 (t, J=6.6 Hz, 2H), 2.14-1.98 (m, 4H); LCMS: C₃₀H₃₀F₃N₇O₂ requires: 578, found: m/z=579 [M+H]⁺.

### Example 653: 6-((4-fluoroisoindolin-2-yl)methyl)-2-(3-(3-((4-methyl-4H-1,2,4-triazol-3-yl)methyl)oxetan-3-yl)phenyl)-4-(trifluoromethyl)isoindolin-1-one

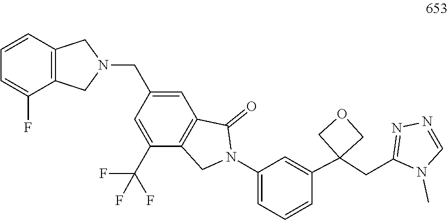 [SMILES: Cn1cnnc1CC1(c2cccc(N3Cc4c(cc(CN5Cc6cccc(F)c6C5)cc4C(F)(F)F)C3=O)c2)COC1]

The reductive amination was carried out in a similar fashion as for 411, step 2 using Example Z (100 mg, 0.22 mmol) and 4-fluoro-2,3-dihydro-1H-isoindole hydrochloride (38 mg, 0.22 mmol) as reactants to afford the title compound (35 mg, 27%) as a white solid. ¹H NMR (500 MHz, Acetonitrile-d₃) δ 8.07 (s, 1H), 8.01 (s, 1H), 7.96-7.89 (m, 2H), 7.38 (dd, J=8.0 Hz, 1H), 7.35 (dd, J=2.0 Hz, 1H), 7.30-7.23 (m, 1H), 7.08 (d, J=7.4 Hz, 1H), 6.97 (dd, J=8.8 Hz, 1H), 6.79 (ddd, J=7.7, 1.8, 1.0 Hz, 1H), 5.05 (d, J=6.1 Hz, 2H), 5.03-4.95 (m, 4H), 4.12 (s, 2H), 4.07-3.98 (m, 4H), 3.55 (s, 2H), 2.89 (s, 3H); LCMS: C₃₁H₂₇F₄N₅O₂ requires: 577, found: m/z=578 [M+H]⁺.

### Example 654: 6-(isoindolin-2-ylmethyl)-2-(3-(3-((4-methyl-4H-1,2,4-triazol-3-yl)methyl)oxetan-3-yl)phenyl)-4-(trifluoromethyl)isoindolin-1-one

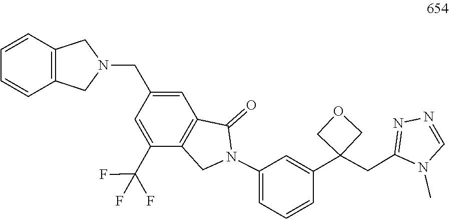 [SMILES: Cn1cnnc1CC1(c2cccc(N3Cc4c(cc(CN5Cc6ccccc6C5)cc4C(F)(F)F)C3=O)c2)COC1]

The reductive amination was carried out in a similar fashion as for 411, step 2 using Example Z (100 mg, 0.22 mmol) and isoindoline (78 mg, 0.66 mmol) as reactants to afford the title compound (24 mg, 20%) as a white solid, ¹H NMR (500 MHz, Acetonitrile-d₃) δ 8.07 (s, 1H), 8.02 (s, 1H), 7.96-7.89 (m, 2H), 7.38 (dd, J=8.0 Hz, 1H), 7.36-7.31 (m, 2H), 7.28-7.20 (m, 3H), 6.79 (ddd, J=7.6, 1.8, 0.9 Hz, 1H), 5.05 (d, J=6.1 Hz, 2H), 4.99 (s, 4H), 4.11 (s, 2H), 3.97 (s, 4H), 3.55 (s, 2H), 2.89 (s, 3H); LCMS: C₃₁H₂₈F₃N₅O₂ requires: 559, found: m/z=560 [M+H]⁺.

### Example 655: 6-((5-fluoroisoindolin-2-yl)methyl)-2-(3-(3-((4-methyl-4H-1,2,4-triazol-3-yl)methyl)oxetan-3-yl)phenyl)-4-(trifluoromethyl)isoindolin-1-one

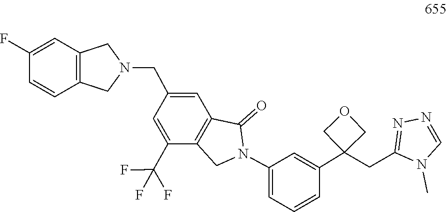 [SMILES: Cn1cnnc1CC1(c2cccc(N3Cc4c(cc(CN5Cc6ccc(F)cc6C5)cc4C(F)(F)F)C3=O)c2)COC1]

The reductive amination was carried out in a similar fashion as for 411, step 2 using Example Z (100 mg, 0.22 mmol) and 5-fluoro-2,3-dihydro-1H-isoindole hydrochloride (114 mg, 0.66 mmol) as reactants to afford the title compound (16 mg, 13%) as a white solid. ¹H NMR (500 MHz, Acetonitrile-d₃) δ 8.06 (s, 1H), 8.01 (s, 1H), 7.96-7.89 (m, 2H), 7.38 (dd, J=8.0 Hz, 1H), 7.35 (dd, J=2.0 Hz, 1H), 7.23 (dd, J=8.3, 5.2 Hz, 1H), 7.04-6.93 (m, 2H), 6.79 (ddd, J=7.6, 1.8, 0.9 Hz, 1H), 5.05 (d, J=6.1 Hz, 2H), 5.00 (d, J=5.9 Hz, 4H), 4.10 (s, 2H), 3.95 (d, J=15.3 Hz, 4H), 3.55 (s, 3H), 2.89 (s, 3H); LCMS: C₃₁H₂₇F₄N₅O₂ requires: 577, found: m/z=578 [M+H]⁺.

### Example 656: 6-((5-methoxyisoindolin-2-yl)methyl)-2-(3-(3-((4-methyl-4H-1,2,4-triazol-3-yl)methyl)oxetan-3-yl)phenyl)-4-(trifluoromethyl)isoindolin-1-one

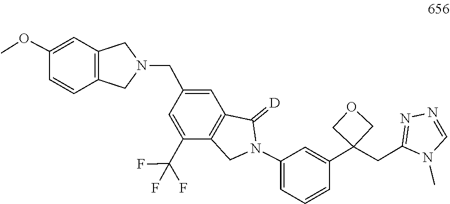

The reductive amination was carried out in a similar fashion as for 411, step 2 using Example Z (100 mg, 0.22 mmol) and 5-methoxy-2,3-dihydro-1H-isoindole hydrochloride (122 mg, 0.66 mmol) as reactants to afford the title compound (47 mg, 36%) as a white solid. ¹H NMR (500 MHz, Acetonitrile-d₃) δ 8.43 (s, 1H), 8.25 (d, J=1.4 Hz, 1H), 8.20 (s, 1H), 7.92 (ddd, J=8.2, 2.3, 0.9 Hz, 1H), 7.44 (dd, J=8.0 Hz, 1H), 7.40 (dd, J=2.0 Hz, 1H), 7.27 (d, J=8.4 Hz, 1H), 6.98-6.91 (m, 2H), 6.88 (dt, J=7.8, 1.1 Hz, 1H), 5.09 (d, J=1.4 Hz, 2H), 5.02 (d, J=1.3 Hz, 3H), 4.71 (s, 2H), 4.60 (s, 3H), 3.81 (s, 3H), 3.74 (s, 2H), 3.02 (s, 3H); LCMS: C₃₂H₃₀F₃N₅O₃ requires: 589, found: m/z=590 [M+H]⁺.

### Example 657: 6-((4-chloroisoindolin-2-yl)methyl)-2-(3-(3-((4-methyl-4H-1,2,4-triazol-3-yl)methyl)oxetan-3-yl)phenyl)-4-(trifluoromethyl)isoindolin-1-one

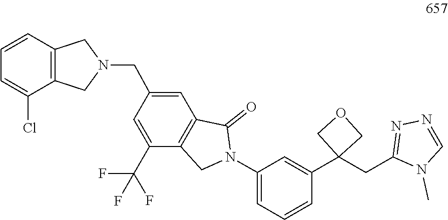 [SMILES: Cn1cnnc1CC1(c2cccc(N3Cc4c(cc(CN5Cc6cccc(Cl)c6C5)cc4C(F)(F)F)C3=O)c2)COC1]

The reductive amination was carried out in a similar fashion as for 411, step 2 using Example Z (100 mg, 0.22 mmol) and 4-chloro-2,3-dihydro-1H-isoindole hydrochloride (124 mg, 0.66 mmol) as reactants to afford the title compound (4 mg, 3%) as a white solid. ¹H NMR (500 MHz, Acetonitrile-d₃) δ 8.08 (s, 1H), 8.02 (s, 1H), 7.96-7.90 (m, 2H), 7.38 (dd, J=8.0 Hz, 1H), 7.35 (dd, J=2.0 Hz, 1H), 7.25 (d, J=4.8 Hz, 2H), 7.20 (q, J=4.5 Hz, 1H), 6.79 (dd, J=7.6, 1.4 Hz, 1H), 5.05 (d, J=6.1 Hz, 2H), 5.00 (d, J=6.1 Hz, 4H), 4.12 (s, 2H), 4.09-4.00 (m, 4H), 3.55 (s, 2H), 2.89 (s, 3H); LCMS: C₃₁H₂₇ClF₃N₅O₂ requires: 594, found: m/z=595 [M+H]⁺.

### Example 658: 2-((2-(3-(3-((4-methyl-4H-1,2,4-triazol-3-yl)methyl)oxetan-3-yl)phenyl)-3-oxo-7-(trifluoromethyl)isoindolin-5-yl)methyl)isoindoline-4-carbonitrile

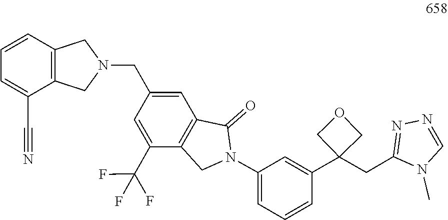 [SMILES: Cn1cnnc1CC1(c2cccc(N3Cc4c(cc(CN5Cc6cccc(C#N)c6C5)cc4C(F)(F)F)C3=O)c2)COC1]

The reductive amination was carried out in a similar fashion as for 411, step 2 using Example Z (100 mg, 0.22 mmol) and 2,3-dihydro-1H-isoindole-4-carbonitrile hydrochloride (120 mg, 0.66 mmol) as reactants to afford the title compound (19 mg, 15%) as a white solid. ¹H NMR (500 MHz, Acetonitrile-d₃) δ 8.08 (s, 1H), 8.02 (s, 1H), 7.96-7.89 (m, 2H), 7.58 (dd, J=7.8, 0.9 Hz, 1H), 7.56-7.51 (m, 1H), 7.40 (dd, J=10.2, 7.8 Hz, 1H), 7.37-7.32 (m, 1H), 6.79 (ddd, J=7.7, 1.8, 0.9 Hz, 1H), 5.05 (d, J=6.1 Hz, 2H), 5.04-4.92 (m, 4H), 4.14 (s, 4H), 4.06 (p, J=1.1 Hz, 2H), 3.55 (s, 2H), 2.89 (s, 3H); LCMS: C₃₂H₂₇F₃N₆O₂ requires: 584, found: m/z=585 [M+H]⁺.

### Example 659: 6-((5-chloroisoindolin-2-yl)methyl)-2-(3-(3-((4-methyl-4H-1,2,4-triazol-3-yl)methyl)oxetan-3-yl)phenyl)-4-(trifluoromethyl)isoindolin-1-one

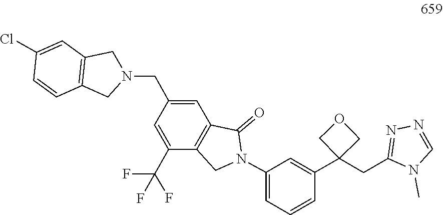 [SMILES: Cn1cnnc1CC1(c2cccc(N3Cc4c(cc(CN5Cc6ccc(Cl)cc6C5)cc4C(F)(F)F)C3=O)c2)COC1]

The reductive amination was carried out in a similar fashion as for 411, step 2 using Example Z (100 mg, 0.22 mmol) and 5-chloro-2,3-dihydro-1H-isoindole hydrochloride (124 mg, 0.66 mmol) as reactants to afford the title compound (27 mg, 21%) as a white solid, ¹H NMR (500 MHz, Acetonitrile-d₃) δ 8.08 (s, 1H), 8.03 (s, 1H), 7.92 (d, J=7.8 Hz, 2H), 7.38 (dd, J=8.0 Hz, 1H), 7.35 (dd, J=2.0 Hz, 1H), 7.28 (s, 1H), 7.27-7.22 (m, 2H), 6.80 (d, J=7.7, 1.2 Hz, 1H), 5.05 (d, J=6.1 Hz, 2H), 5.04-4.95 (m, 4H), 4.13 (s, 2H), 3.98 (d, J=6.0 Hz, 4H), 3.55 (s, 2H), 2.89 (s, 3H); LCMS: C₃₁H₂₇ClF₃N₅O₂ requires: 594, found: m/z=595 [M+H]⁺.

### Example 660: 6-((5,7-dihydro-6H-pyrrolo[3,4-b]pyridin-6-yl)methyl)-2-(3-(3-((4-methyl-4H-1,2,4-triazol-3-yl)methyl)oxetan-3-yl)phenyl)-4-(trifluoromethyl)isoindolin-1-one

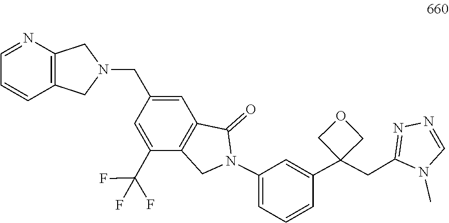 [SMILES: Cn1cnnc1CC1(c2cccc(N3Cc4c(cc(CN5Cc6cccnc6C5)cc4C(F)(F)F)C3=O)c2)COC1]

The reductive amination was carried out in a similar fashion as for 411, step 2 using Example Z (100 mg, 0.22 mmol) and 5H,6H,7H-pyrrolo[3,4-b]pyridine hydrochloride (103 mg, 0.66 mmol) as reactants to afford the title compound (16 mg, 13%) as a white solid. ¹H NMR (500 MHz, Acetonitrile-d₃) δ 8.42-8.33 (m, 1H), 8.08 (s, 1H), 8.02 (s, 1H), 7.96-7.89 (m, 1H), 7.60 (dd, J=7.6, 1.4 Hz, 1H), 7.38 (dd, J=8.0 Hz, 1H), 7.35 (dd, J=2.0 Hz, 1H), 7.18 (dd, J=7.6, 5.0 Hz, 1H), 6.85-6.74 (m, 1H), 5.05 (d, J=6.1 Hz, 2H), 5.03-4.95 (m, 3H), 4.13 (s, 2H), 4.04-3.95 (m, 2H), 3.55 (s, 2H), 2.89 (s, 3H); LCMS: C₃₀H₂₇F₃N₆O₂ requires: 560, found: m/z=561 [M+H]⁺.

### Example 661: 6-((7-methoxy-3,4-dihydroisoquinolin-2(1H)-yl)methyl)-2-(3-(3-((4-methyl-4H-1,2,4-triazol-3-yl)methyl)oxetan-3-yl)phenyl)-4-(trifluoromethyl)isoindolin-1-one

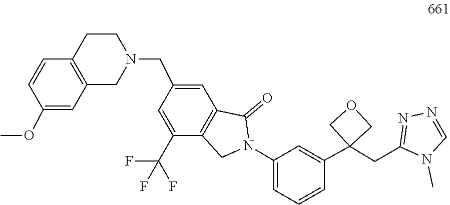 [SMILES: COc1ccc2c(c1)CN(Cc1cc3c(c(C(F)(F)F)c1)CN(c1cccc(C4(Cc5nncn5C)COC4)c1)C3=O)CC2]

The reductive amination was carried out in a similar fashion as for 411, step 2 using Example Z (100 mg, 0.22 mmol) and 7-methoxy-1,2,3,4-tetrahydroisoquinoline (107 mg, 0.66 mmol) as reactants to afford the title compound (82 mg, 62%) as a white solid. ¹H NMR (500 MHz, Acetonitrile-d₃) δ 8.06 (s, 1H), 8.00 (s, 1H), 7.95-7.89 (m, 2H), 7.38 (dd, J=8.0 Hz, 1H), 7.34 (dd, J=2.0 Hz, 1H), 7.06 (d, J=8.4 Hz, 1H), 6.79 (d, J=7.7, 1.3 Hz, 1H), 6.75 (dd, J=8.4, 2.7 Hz, 1H), 6.60 (d, J=2.7 Hz, 1H), 5.05 (d, J=6.0 Hz, 2H), 5.00 (d, J=6.0 Hz, 4H), 3.87 (s, 2H), 3.74 (s, 3H), 3.63 (s, 2H), 3.55 (s, 2H), 2.89 (s, 3H), 2.86-2.75 (m, 4H); LCMS: C₃₃H₃₂F₃N₅O₃ requires: 604, found: m/z=605 [M+H]⁺.

### Example 662a: (R)-2-(3-(3-((4-methyl-4H-1,2,4-triazol-3-yl)methyl)oxetan-3-yl)phenyl)-6-((3-phenylpyrrolidin-1-yl)methyl)-4-(trifluoromethyl)isoindolin-1-one

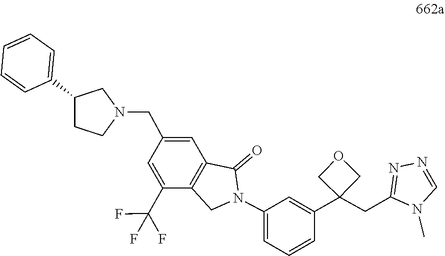 [SMILES: Cn1cnnc1CC1(c2cccc(N3Cc4c(cc(CN5CC[C@H](c6ccccc6)C5)cc4C(F)(F)F)C3=O)c2)COC1]

The reductive amination was carried out in a similar fashion as for 411, step 2 using Example Z (100 mg, 0.22 mmol) and (3R)-3-phenylpyrrolidine hydrochloride (121 mg, 0.66 mmol) as reactants to afford the title compound (77 mg, 60%) as a white solid. ¹H NMR (500 MHz, Acetonitrile-d₃) δ 8.05 (s, 1H), 8.00 (s, 1H), 7.95-7.89 (m, 2H), 7.41-7.29 (m, 6H), 7.26-7.19 (m, 1H), 6.79 (dt, J=7.8, 1.3 Hz, 1H), 5.04 (d, J=6.0 Hz, 2H), 5.02-4.97 (m, 4H), 3.93 (s, 3H), 3.55 (s, 2H), 3.48-3.38 (m, 1H), 3.04 (d, J=10.2 Hz, 1H), 2.67 (s, 1H), 2.43-2.33 (m, 2H), 1.95-1.84 (m, 2H); LCMS: C₃₃H₃₂F₃N₅O₂ requires: 587, found: m/z=588 [M+H]⁺.

### Example 662b: (S)-2-(3-(3-((4-methyl-4H-1,2,4-triazol-3-yl)methyl)oxetan-3-yl)phenyl)-6-((3-phenylpyrrolidin-1-yl)methyl)-4-(trifluoromethyl)isoindolin-1-one

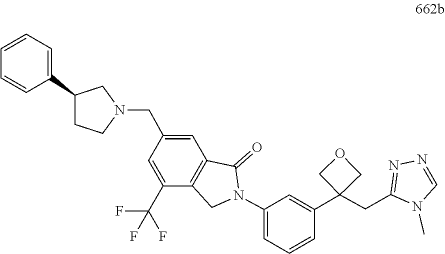 [SMILES: Cn1cnnc1CC1(c2cccc(N3Cc4c(cc(CN5CC[C@@H](c6ccccc6)C5)cc4C(F)(F)F)C3=O)c2)COC1]

The reductive amination was carried out in a similar fashion as for 411, step 2 using Example Z (100 mg, 0.22 mmol) and (3S)-3-phenylpyrrolidine hydrochloride (121 mg, 0.66 mmol) as reactants to afford the title compound (47 mg, 37%) as a white solid. ¹H NMR (500 MHz, Acetonitrile-d₃) δ 8.05 (s, 1H), 8.00 (s, 1H), 7.96-7.90 (m, 2H), 7.45-7.31 (m, 6H), 7.27-7.19 (m, 1H), 6.79 (dt, J=7.8, 1.3 Hz, 1H), 5.04 (d, J=6.0 Hz, 2H), 5.01-4.99 (m, 4H), 3.93 (s, 3H), 3.55 (s, 2H), 3.49-3.40 (m, 1H), 3.04 (d, J=10.2 Hz, 1H), 2.67 (s, 1H), 2.44-2.34 (m, 2H), 1.97-1.86 (m, 2H); LCMS: C₃₃H₃₂F₃N₅O₂ requires: 587, found: m/z=588 [M+H]⁺.

### Example 663a: (S)-2-(3-(3-((4-methyl-4H-1,2,4-triazol-3-yl)methyl)oxetan-3-yl)phenyl)-6-((2-methylmorpholino)methyl)-4-(trifluoromethyl)isoindolin-1-one

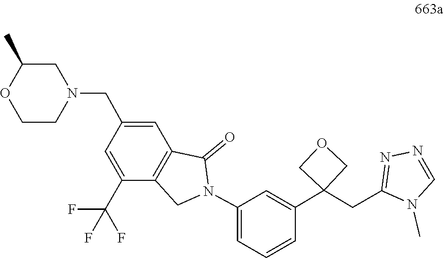 [SMILES: C[C@H]1CN(Cc2cc3c(c(C(F)(F)F)c2)CN(c2cccc(C4(Cc5nncn5C)COC4)c2)C3=O)CCO1]

The reductive amination was carried out in a similar fashion as for 411, step 2 using Example Z (200 mg, 0.44 mmol) and (2S)-2-methylmorpholine hydrochloride (121 mg, 0.88 mmol) as reactants to afford the title compound (59 mg, 25%) as a white solid ¹H NMR (500 MHz, Acetonitrile-d₃) δ 8.01 (s, 1H), 7.95-7.89 (m, 2H), 7.38 (dd, J=8.0 Hz, 1H), 7.34 (dd, J=2.0 Hz, 1H), 6.79 (ddd, J=7.7, 1.7, 0.9 Hz, 1H), 5.05 (d, J=6.1 Hz, 2H), 5.03-4.93 (m, 3H), 3.84-3.76 (m, 1H), 3.68 (d, J=1.9 Hz, 2H), 3.65-3.57 (m, 2H), 3.55 (s, 2H), 2.89 (s, 3H), 2.73 (dt, J=11.2, 2.1 Hz, 1H), 2.68-2.63 (m, 1H), 1.87 (dd, J=11.2, 9.9 Hz, 1H), 1.09 (d, J=6.3 Hz, 3H); LCMS: C₂₈H₃₀F₃N₅O₃ requires: 541, found: m/z=542 [M+H]⁺.

Example 663b: (R)-2-(3-(3-((4-methyl-4H-1,2,4-triazol-3-yl)methyl)oxetan-3-yl)phenyl)-6-((2-methylmorpholino)methyl)-4-(trifluoromethyl)isoindolin-1-one

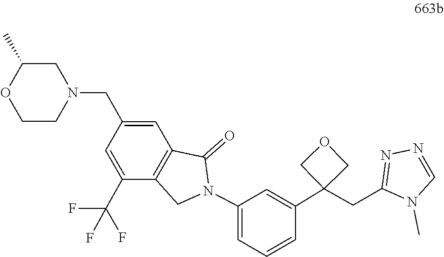 [SMILES: C[C@@H]1CN(Cc2cc3c(c(C(F)(F)F)c2)CN(c2cccc(C4(Cc5nncn5C)COC4)c2)C3=O)CCO1]

The reductive amination was carried out in a similar fashion as for 411, step 2 using Example Z (200 mg, 0.44 mmol) and (2R)-2-methylmorpholine hydrochloride (121 mg, 0.88 mmol) as reactants to afford the title compound (53 mg, 23%) as a white solid. ¹H NMR (500 MHz, Acetonitrile-d₃) δ 8.01 (s, 1H), 7.94-7.91 (m, 2H), 7.38 (dd, J=8.0 Hz, 1H), 7.34 (dd, J=2.0 Hz, 1H), 6.79 (ddd, J=7.7, 1.7, 0.9 Hz, 1H), 5.05 (d, J=6.1 Hz, 2H), 5.04-4.90 (m, 3H), 3.86-3.76 (m, 1H), 3.68 (d, J=1.9 Hz, 2H), 3.66-3.58 (m, 2H), 3.55 (s, 2H), 2.89 (s, 3H), 2.73 (dt, J=11.2, 2.1 Hz, 1H), 2.69-2.65 (m, 1H), 1.87 (dd, J=11.2, 9.9 Hz, 1H), 1.09 (d, J=6.3 Hz, 3H); LCMS: C₂₈H₃₀F₃N₅O₃ requires: 541, found: m/z=542 [M+H]⁺.

### Example 664: 6-(((cis)-2,6-dimethylmorpholino)methyl)-2-(3-(3-((4-methyl-4H-1,2,4-triazol-3-yl)methyl)oxetan-3-yl)phenyl)-4-(trifluoromethyl)isoindolin-1-one

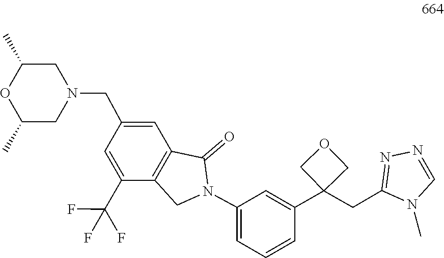 [SMILES: C[C@@H]1CN(Cc2cc3c(c(C(F)(F)F)c2)CN(c2cccc(C4(Cc5nncn5C)COC4)c2)C3=O)C[C@H](C)O1]

The reductive amination was carried out in a similar fashion as for 411, step 2 using Example Z (100 mg, 0.22 mmol) and (cis)-2,6-dimethylmorpholine (76 mg, 0.66 mmol) as reactants to afford the title compound (75 mg, 62%) as a white solid. ¹H NMR (500 MHz, Acetonitrile-d₃) δ 8.00 (s, 1H), 7.92 (d, J=7.5 Hz, 3H), 7.38 (dd, J=8.0 Hz, 1H), 7.34 (d, J=2.0 Hz, 1H), 6.79 (d, J=7.7 Hz, 1H), 5.05 (d, J=6.0 Hz, 2H), 5.00 (d, J=6.4 Hz, 4H), 3.71-3.61 (m, 4H), 3.55 (s, 2H), 2.79-2.65 (m, 2H), 1.79 (t, J=10.6 Hz, 2H), 1.09 (d, J=6.4, 1.5 Hz, 6H); LCMS: C₂₉H₃₂F₃N₅O₃ requires: 555, found: m/z=556 [M+H]⁺.

### Example 665: 6-((2,2-dimethylmorpholino)methyl)-2-(3-(3-((4-methyl-4H-1,2,4-triazol-3-yl)methyl)oxetan-3-yl)phenyl)-4-(trifluoromethyl)isoindolin-1-one

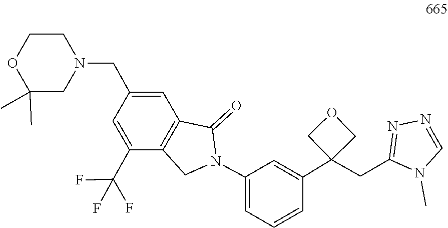 [SMILES: Cn1cnnc1CC1(c2cccc(N3Cc4c(cc(CN5CCOC(C)(C)C5)cc4C(F)(F)F)C3=O)c2)COC1]

The reductive amination was carried out in a similar fashion as for 411, step 2 using Example Z (100 mg, 0.22 mmol) and 2,2-dimethylmorpholine (76 mg, 0.66 mmol) as reactants to afford the title compound (97 mg, 80%) as a white solid. ¹H NMR (500 MHz, Acetonitrile-d₃) δ 8.02 (s, 1H), 7.97 (s, 1H), 7.95-7.87 (m, 2H), 7.38 (dd, J=8.0 Hz, 1H), 7.35 (dd, J=2.0 Hz, 1H), 6.79 (d, J=7.7, 1.2 Hz, 1H), 5.05 (d, J=6.1 Hz, 2H), 4.99 (d, J=6.3 Hz, 4H), 3.75-3.69 (m, 2H), 3.55 (s, 2H), 2.89 (s, 3H), 2.45-2.36 (m, 2H), 2.24 (s, 2H), 1.22 (s, 6H); LCMS: C₂₉H₃₂F₃N₅O₃ requires: 555, found: m/z=556 [M+H]⁺.

### Example 666: 6-((4-oxa-7-azaspiro[2.5]octan-7-yl)methyl)-2-(3-(3-((4-methyl-4H-1,2,4-triazol-3-yl)methyl)oxetan-3-yl)phenyl)-4-(trifluoromethyl)isoindolin-1-one

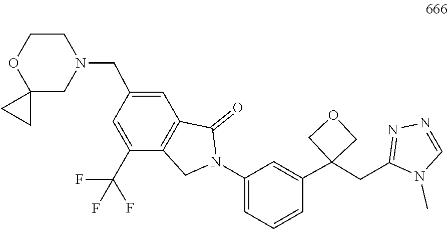 [SMILES: Cn1cnnc1CC1(c2cccc(N3Cc4c(cc(CN5CCOC6(CC6)C5)cc4C(F)(F)F)C3=O)c2)COC1]

The reductive amination was carried out in a similar fashion as for 411, step 2 using Example Z (100 mg, 0.22 mmol) and 4-oxa-7-azaspiro[2.5]octane hydrochloride (98 mg, 0.66 mmol) as reactants to afford the title compound (65 mg, 54%) as a white solid. ¹H NMR (500 MHz, Acetonitrile-d₃) δ 8.13-7.95 (m, 2H), 7.96-7.88 (m, 2H), 7.38 (dd, J=8.0 Hz, 1H), 7.34 (dd, J=2.0 Hz, 1H), 6.80 (d, J=7.7, 1.2 Hz, 1H), 5.05 (d, J=6.0 Hz, 2H), 5.04-4.87 (m, 4H), 3.97-3.63 (m, 4H), 3.55 (s, 2H), 2.89 (s, 3H), 2.70-2.35 (m, 4H), 0.86-0.68 (m, 2H), 0.63-0.38 (m, 2H); LCMS: C₂₉H₃₀F₃N₅O₃ requires: 554, found: m/z=555 [M+H]⁺.

### Example 667: (S)-6-((2-(methoxymethyl)morpholino)methyl)-2-(3-(3-((4-methyl-4H-1,2,4-triazol-3-yl)methyl)oxetan-3-yl)phenyl)-4-(trifluoromethyl)isoindolin-1-one

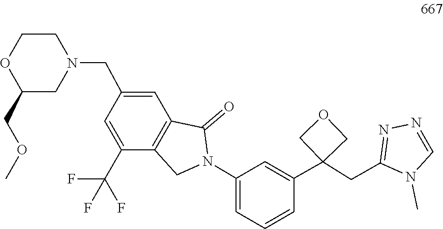 [SMILES: COC[C@@H]1CN(Cc2cc3c(c(C(F)(F)F)c2)CN(c2cccc(C4(Cc5nncn5C)COC4)c2)C3=O)CCO1]

The reductive amination was carried out in a similar fashion as for 411, step 2 using Example Z (100 mg, 0.22 mmol) and (2S)-2-(methoxymethyl)morpholine hydrochloride (110 mg, 0.66 mmol) as reactants to afford the title compound (81 mg, 65%) as a white solid, ¹H NMR (500 MHz, Acetonitrile-d₃) δ 8.04 (s, 1H), 7.97 (s, 1H), 7.92 (d, J=5.1 Hz, 2H), 7.38 (dd, J=8.0 Hz, 1H), 7.34 (dd, J=2.0 Hz, 1H), 6.79 (d, J=7.7, 1.2 Hz, 1H), 5.05 (d, J=5.9 Hz, 2H), 5.03-4.92 (m, 4H), 3.86 (d, J=10.9 Hz, 2H), 3.82-3.62 (m, 4H), 3.55 (s, 2H), 3.39 (dd, J=10.2, 5.6 Hz, 1H), 3.32 (dd, J=10.3, 5.0 Hz, 1H), 3.29 (s, 3H), 2.89 (s, 3H), 2.75 (s, 3H), 2.83-2.68 (m, 2H); LCMS: C₂₉H₃₂F₃N₅O₄ requires: 571, found: m/z=572 [M+H]⁺.

### Example 668a and 668b: (R)-6-(1-(3-fluoroazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one and (S)-6-(1-(3-fluoroazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one

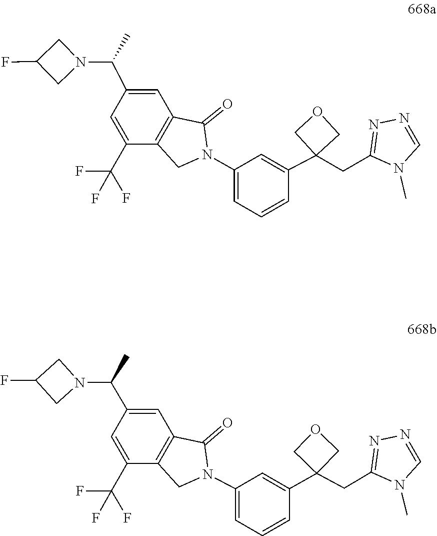 [SMILES: C[C@H](c1cc2c(c(C(F)(F)F)c1)CN(c1cccc(C3(Cc4nncn4C)COC3)c1)C2=O)N1CC(F)C1 ; C[C@@H](c1cc2c(c(C(F)(F)F)c1)CN(c1cccc(C3(Cc4nncn4C)COC3)c1)C2=O)N1CC(F)C1]

Step 1: Synthesis of 6-(1-(3-fluoroazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one. Reductive amination of the methyl ketone (XX) was carried out according to the procedure for 459, step 2 using 6-acetyl-2-(3-(3-((4-methyl-4H-1,2,4-triazol-3-yl)methyl)oxetan-3-yl)phenyl)-4-(trifluoro-methyl)isoindolin-1-one (70 mg, 0.15 mmol) and 3-fluoroazetidine hydrochloride (66 mg, 0.60 mmol) as reactants to afford the title compound (45 mg, 57% yield) as a white solid.

Step 2: (R)-6-(1-(3-fluoroazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one and (S)-6-(1-(3-fluoroazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one. 6-(1-(3-fluoroazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one was separated using IG column with CO₂ and methanol as mobile phase to afford (R)-6-(1-(3-fluoroazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one (14 mg) and (R)-6-(1-(3-fluoroazetidin-1-yl)ethyl)-2-(3-(3-((4-methyl-4H-1,2,4-triazol-3-yl)methyl)oxetan-3-yl)phenyl)-4-(trifluoromethyl)isoindolin-1-one (14 mg).

(R)-6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one. ¹H NMR (500 MHz, DMSO-d₆) δ 8.19 (s, 1H), 8.00 (s, 1H), 7.94 (s, 1H), 7.89 (ddd, J=8.3, 2.2, 0.9 Hz, 1H), 7.41-7.33 (m, 2H), 6.78 (dt, J=7.8, 1.2 Hz, 1H), 5.25 (p, J=5.0 Hz, 1H), 5.11-5.07 (m, 2H), 4.97 (d, J=6.1 Hz, 2H), 4.90 (d, J=6.0 Hz, 2H), 3.68 (dq, J=21.8, 8.2, 7.3 Hz, 2H), 3.52 (s, 2H), 3.37 (dt, J=15.2, 7.1 Hz, 1H), 3.21-3.03 (m, 2H), 2.91 (s, 3H), 1.21 (d, J=6.5 Hz, 3H); LCMS: C₂₇H₂₇F₄N₅O₂ requires: 529, found: m/z=530 [M+H]⁺.

(S)-6-(1-(3,3-difluoropyrrolidin-1-yl)ethyl)-2-(3-(3-((4-methyl-4H-1,2,4-triazol-3-yl)methyl)oxetan-3-yl)phenyl)-4-(trifluoromethyl)isoindolin-1-one. ¹H NMR (500 MHz, DMSO-d₆) δ 8.19 (s, 1H), 8.00 (s, 1H), 7.94 (s, 1H), 7.89 (ddd, J=8.3, 2.2, 0.9 Hz, 1H), 7.41-7.33 (m, 2H), 6.78 (dt, J=7.8, 1.2 Hz, 1H), 5.25 (p, J=5.0 Hz, 1H), 5.10-5.09 (m, 2H), 4.97 (d, J=6.1 Hz, 2H), 4.90 (d, J=6.0 Hz, 2H), 3.68 (dq, J=21.8, 8.2, 7.3 Hz, 2H), 3.52 (s, 2H), 3.37 (dt, J=15.2, 7.1 Hz, 1H), 3.20-3.05 (m, 2H), 2.91 (s, 3H), 1.21 (d, J=6.5 Hz, 3H); LCMS: C₂₇H₂₇F₄N₅O₂ requires: 529, found: m/z=530 [M+H]⁺.

### Example 669a and 669b: (S)-2-cyclopropyl-N-(3-(3-(fluoro(4-methyl-4H-1,2,4-triazol-3-yl)methyl)oxetan-3-yl)phenyl)-6-methylpyrimidine-4-carboxamide and (R)-2-cyclopropyl-N-(3-(3-(fluoro(4-methyl-4H-1,2,4-triazol-3-yl)methyl)oxetan-3-yl)phenyl)-6-methylpyrimidine-4-carboxamide

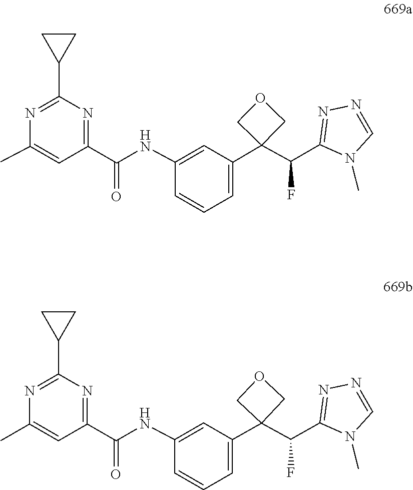 [SMILES: Cc1cc(C(=O)Nc2cccc(C3([C@H](F)c4nncn4C)COC3)c2)nc(C2CC2)n1 ; Cc1cc(C(=O)Nc2cccc(C3([C@@H](F)c4nncn4C)COC3)c2)nc(C2CC2)n1]

Step 1: Synthesis of 2-cyclopropyl-N-(3-(3-(fluoro(4-methyl-4H-1,2,4-triazol-3-yl)methyl)oxetan-3-yl)phenyl)-6-methylpyrimidine-4-carboxamide. The amide bond formation reaction was carried out in a similar fashion as for 184 using 3-{[3-(3-bromophenyl)oxetan-3-yl](fluoro)methyl}-4-methyl-1,2,4-triazole (301 mg, 0.92 mmol) and 2-cyclopropyl-6-methylpyrimidine-4-carboxamide (164 mg, 0.92 mmol) as reactants afforded the title compound (250 mg, 64%) as an off-white solid.

Step 2: (S)-2-cyclopropyl-N-(3-(3-(fluoro(4-methyl-4H-1,2,4-triazol-3-yl)methyl)-oxetan-3-yl)phenyl)-6-methylpyrimidine-4-carboxamide and (R)-2-cyclopropyl-N-(3-(3-(fluoro(4-methyl-4H-1,2,4-triazol-3-yl)methyl)oxetan-3-yl)phenyl)-6-methylpyrimidine-4-carboxamide. 2-cyclopropyl-N-(3-(3-(fluoro(4-methyl-4H-1,2,4-triazol-3-yl)methyl)oxetan-3-yl)phenyl)-6-methylpyrimidine-4-carboxamide was separated using IG column with CO₂ and methanol as mobile phase to afford (S)-2-cyclopropyl-N-(3-(3-(fluoro(4-methyl-4H-1,2,4-triazol-3-yl)methyl)oxetan-3-yl)phenyl)-6-methylpyrimidine-4-carboxamide (66 mg) and (R)-2-cyclopropyl-N-(3-(3-(fluoro(4-methyl-4H-1,2,4-triazol-3-yl)methyl)oxetan-3-yl)phenyl)-6-methylpyrimidine-4-carboxamide (107 mg).

(S)-2-cyclopropyl-N-(3-(3-(fluoro(4-methyl-4H-1,2,4-triazol-3-yl)methyl)oxetan-3-yl)phenyl)-6-methylpyrimidine-4-carboxamide. ¹H NMR (500 MHz, DMSO-d₆) δ 10.36 (s, 1H), 8.30 (s, 1H), 7.76 (dt, J=8.1, 1.3 Hz, 1H), 7.64 (s, 1H), 7.55 (t, J=1.9 Hz, 1H), 7.25 (t, J=8.0 Hz, 1H), 6.83 (dd, J=7.7, 1.2 Hz, 1H), 6.19 (d, J=45.9 Hz, 1H), 5.26 (d, J=6.7 Hz, 1H), 5.12 (dd, J=6.1, 1.4 Hz, 1H), 5.03 (dd, J=6.8, 1.9 Hz, 1H), 4.72 (dd, J=6.2, 4.0 Hz, 1H),3.15 (s, 3H), 2.45 (s, 1H), 2.34-2.16 (m, 3H), 1.20-0.96 (m, 4H); LCMS: C₂₂H₂₃FN₆O₂ requires: 422, found: m/z=423 [M+H]⁺.

(R)-2-cyclopropyl-N-(3-(3-(fluoro(4-methyl-4H-1,2,4-triazol-3-yl)methyl)oxetan-3-yl)phenyl)-6-methylpyrimidine-4-carboxamide. ¹H NMR (500 MHz, DMSO-d₆) δ 10.36 (s, 1H), 8.30 (s, 1H), 7.76 (dt, J=8.1, 1.3 Hz, 1H), 7.64 (s, 1H), 7.55 (t, J=1.9 Hz, 1H), 7.25 (t, J=8.0 Hz, 1H), 6.83 (dd, J=7.7, 1.2 Hz, 1H), 6.19 (d, J=45.9 Hz, 1H), 5.26 (d, J=6.7 Hz, 1H), 5.12 (dd, J=6.1, 1.4 Hz, 1H), 5.03 (dd, J=6.8, 1.9 Hz, 1H), 4.72 (dd, J=6.2, 4.0 Hz, 1H), 3.15 (s, 3H), 2.45 (s, 1H), 2.32-2.17 (m, 3H), 1.18-0.94 (m, 4H); LCMS: C₂₂H₂₃FN₆O₂ requires: 422, found: m/z=423 [M+H]⁺.

### Example 670a: (S)-2-(3-(3-(fluoro(4-methyl-4H-1,2,4-triazol-3-yl)methyl)oxetan-3-yl)phenyl)-6-((4-fluoropiperidin-1-yl)methyl)-4-(trifluoromethyl)isoindolin-1-one

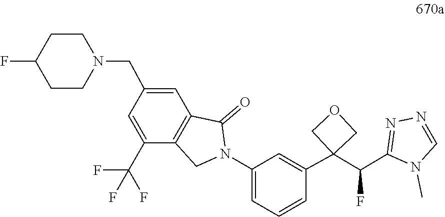 [SMILES: Cn1cnnc1[C@@H](F)C1(c2cccc(N3Cc4c(cc(CN5CCC(F)CC5)cc4C(F)(F)F)C3=O)c2)COC1]

Step 1: Synthesis of (S)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde: The indolone formation reaction was carried out in a manner similar to 260, step 2 using (S)-3-(3-(fluoro(4-methyl-4H-1,2,4-triazol-3-yl)methyl)oxetan-3-yl)aniline AG (81 mg, 0.31 mmol) and methyl 5-bromo-2-(bromomethyl)-3-(trifluoromethyl)benzoate (M, step 2) (100 mg, 0.31 mmol) as reactants to afford the title compound in (40 mg, 27%).

Step 2: Synthesis of (S)-2-(3-(3-(fluoro(4-methyl-4H-1,2,4-triazol-3-yl)methyl)oxetan-3-yl)phenyl)-6-((4-fluoropiperidin-1-yl)methyl)-4-(trifluoromethyl)isoindolin-1-one: The reductive amination was carried out in a similar fashion as for 411, step 2 using (S)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (120 mg, 0.25 mmol) and 4-fluoropiperidine hydrochloride (106 mg, 0.76 mmol) as reactants to afford the title compound (56 mg, 39%) as a white solid. ¹H NMR (500 MHz, Acetonitrile-d₃) δ 7.90 (d, J=6.3 Hz, 2H), 7.87-7.77 (m, 2H), 7.40 (t, J=2.0 Hz, 1H), 7.30 (t, J=8.0 Hz, 1H), 6.85 (dt, J=7.9, 1.1 Hz, 1H), 6.05 (d, J=46.2 Hz, 1H), 5.33 (dd, J=6.7, 1.2 Hz, 1H), 5.17 (dd, J=6.2, 1.0 Hz, 1H), 5.08 (dd, J=6.7, 2.0 Hz, 1H), 4.92-4.87 (m, 1H), 4.84 (dd, J=6.3, 4.3 Hz, 2H), 4.60 (ddt, J=48.9, 7.1, 3.5 Hz, 1H), 3.59 (s, 2H), 3.02 (d, J=0.8 Hz, 3H), 2.59-2.44 (m, 2H), 2.35-2.21 (m, 2H), 1.78-1.64 (m, 4H); LCMS: C₂₉H₂₉F₃N₆O₂ requires: 550, found: m/z=551 [M+H]⁺.

### Example 670b: (R)-2-(3-(3-(fluoro(4-methyl-4H-1,2,4-triazol-3-yl)methyl)oxetan-3-yl)phenyl)-6-((4-fluoropiperidin-1-yl)methyl)-4-(trifluoromethyl)isoindolin-1-one

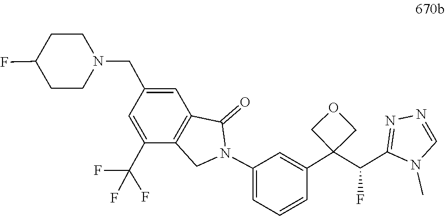 [SMILES: Cn1cnnc1[C@H](F)C1(c2cccc(N3Cc4c(cc(CN5CCC(F)CC5)cc4C(F)(F)F)C3=O)c2)COC1]

Step 1: Synthesis of (R)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde: The indolone formation reaction was carried out in a manner similar to 260, step 2 using (R)-3-(3-(fluoro(4-methyl-4H-1,2,4-triazol-3-yl)methyl)oxetan-3-yl)aniline AH (81 mg, 0.31 mmol) and methyl 5-bromo-2-(bromomethyl)-3-(trifluoromethyl)benzoate (M, step 2) (100 mg, 0.31 mmol) as reactants to afford the title compound in (70 mg, 48%).

Step 2: Synthesis of (R)-2-(3-(3-(fluoro(4-methyl-4H-1,2,4-triazol-3-yl)methyl)oxetan-3-yl)phenyl)-6-((4-fluoropiperidin-1-yl)methyl)-4-(trifluoromethyl)isoindolin-1-one: The reductive amination was carried out in a similar fashion as for 411, step 2 using (R)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (120 mg, 0.25 mmol) and 4-fluoropiperidine hydrochloride (106 mg, 0.76 mmol) as reactants to afford the title compound (79 mg, 55%) as a white solid. ¹H NMR (500 MHz, Acetonitrile-d₃) δ 7.90 (d, J=6.3 Hz, 2H), 7.87-7.78 (m, 2H), 7.40 (t, J=2.0 Hz, 1H), 7.30 (t, J=8.0 Hz, 1H), 6.85 (dt, J=7.9, 1.1 Hz, 1H), 6.05 (d, J=46.2 Hz, 1H), 5.33 (dd, J=6.7, 1.2 Hz, 1H), 5.17 (dd, J=6.2, 1.0 Hz, 1H), 5.08 (dd, J=6.7, 2.0 Hz, 1H), 4.92-4.87 (m, 1H), 4.84 (dd, J=6.3, 4.3 Hz, 2H), 4.60 (ddt, J=48.9, 7.1, 3.5 Hz, 1H), 3.59 (s, 2H), 3.02 (d, J=0.8 Hz, 3H), 2.60-2.46 (m, 2H), 2.35-2.22 (m, 2H), 1.77-1.63 (m, 4H); LCMS: C₂₉H₂₉F₃N₆O₂ requires: 550, found: m/z=551 [M+H]⁺.

### Example 671a: (S)-6-((7-azabicyclo[2.2.1]heptan-7-yl)methyl)-2-(3-(3-(fluoro(4-methyl-4H-1,2,4-triazol-3-yl)methyl)oxetan-3-yl)phenyl)-4-(trifluoromethyl)isoindolin-1-one

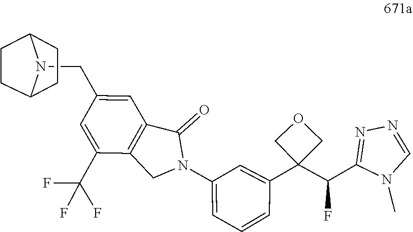 [SMILES: Cn1cnnc1[C@@H](F)C1(c2cccc(N3Cc4c(cc(CN5C6CCC5CC6)cc4C(F)(F)F)C3=O)c2)COC1]

The reductive amination was carried out in a similar fashion as for 411, step 2 using (S)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (50 mg, 0.11 mmol) and 7-azabicyclo[2.2.1]heptane hydrochloride (42 mg, 0.32 mmol) as reactants to afford the title compound (13 mg, 23%) as a white solid. ¹H NMR (500 MHz, Methanol-d₄) δ 8.27 (s, 1H), 8.11-8.07 (m, 1H), 8.02 (s, 1H), 7.79 (dd, J=8.2, 2.2 Hz, 1H), 7.51 (t, J=2.0 Hz, 1H), 7.42 (t, J=8.0 Hz, 1H), 6.92 (d, J=7.8 Hz, 1H), 6.30 (d, J=45.3 Hz, 2H), 5.47 (d, J=6.7 Hz, 1H), 5.31 (d, J=6.4 Hz, 1H), 5.21 (dd, J=6.7, 1.9 Hz, 2H), 5.12-5.07 (m, 2H), 5.01 (dd, J=6.3, 4.1 Hz, 1H), 3.76 (s, 2H), 3.11 (s, 3H), 1.93-1.81 (m, 4H), 1.47-1.35 (m, 4H); LCMS: C₂₉H₂₉F₄N₅O₂ requires: 555, found: m/z=556 [M+H]⁺.

### Example 671b: (R)-6-((7-azabicyclo[2.2.1]heptan-7-yl)methyl)-2-(3-(3-(fluoro(4-methyl-4H-1,2,4-triazol-3-yl)methyl)oxetan-3-yl)phenyl)-4-(trifluoromethyl)isoindolin-1-one

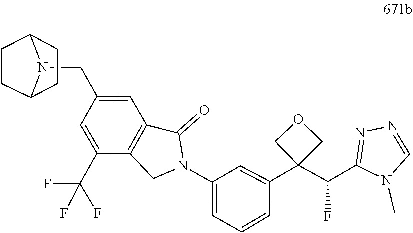 [SMILES: Cn1cnnc1[C@H](F)C1(c2cccc(N3Cc4c(cc(CN5C6CCC5CC6)cc4C(F)(F)F)C3=O)c2)COC1]

The reductive amination was carried out in a similar fashion as for 411, step 2 using (R)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (50 mg, 0.11 mmol) and 7-azabicyclo[2.2.1]heptane hydrochloride (14 mg, 0.11 mmol) as reactants to afford the title compound (25 mg, 43%) as a white solid. ¹H NMR (500 MHz, Methanol-d₄) δ 8.27 (s, 1H), 8.13-8.08 (m, 1H), 8.02 (s, 1H), 7.79 (dd, J=8.2, 2.2 Hz, 1H), 7.51 (t, J=2.0 Hz, 1H), 7.42 (t, J=8.0 Hz, 1H), 6.92 (d, J=7.8 Hz, 1H), 6.30 (d, J=45.3 Hz, 2H), 5.47 (d, J=6.7 Hz, 1H), 5.31 (d, J=6.4 Hz, 1H), 5.21 (dd, J=6.7, 1.9 Hz, 2H), 5.14-5.08 (m, 2H), 5.01 (dd, J=6.3, 4.1 Hz, 1H), 3.76 (s, 2H), 3.11 (s, 3H), 1.96-1.85 (m, 4H), 1.47-1.36 (m, 4H); LCMS: C₂₉H₂₉F₄N₅O₂ requires: 555, found: m/z=556 [M+H]⁺.

### Example 672a: 2-(3-(3-((S)-fluoro(4-methyl-4H-1,2,4-triazol-3-yl)methyl)oxetan-3-yl)-phenyl)-6-(((1R,3S,5S)-3-fluoro-8-azabicyclo[3.2.1]octan-8-yl)methyl)-4-(trifluoromethyl)isoindolin-1-one

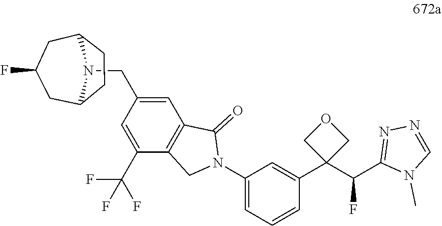

The reductive amination was carried out in a similar fashion as for 411, step 2 using (S)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (50 mg, 0.11 mmol) and (exo)-3-fluoro-8-azabicyclo[3.2.1]octane hydrochloride (52 mg, 0.32 mmol) as reactants to afford the title compound (17 mg, 28%) as a white solid. ¹H NMR (500 MHz, Methanol-d₄) δ 8.27 (s, 1H), 8.12 (s, 1H), 8.02 (s, 1H), 7.79 (ddd, J=8.3, 2.2, 0.9 Hz, 1H), 7.51 (t, J=2.0 Hz, 1H), 7.42 (t, J=8.0 Hz, 1H), 6.92 (dt, J=7.8, 1.2 Hz, 1H), 6.30 (d, J=45.3 Hz, 2H), 5.47 (d, J=6.7 Hz, 2H), 5.31 (d, J=6.2 Hz, 1H), 5.21 (dd, J=6.7, 2.0 Hz, 1H), 5.11-5.06 (m, 2H), 5.01 (dd, J=6.3, 4.1 Hz, 1H), 3.83 (s, 2H), 3.11 (d, J=0.9 Hz, 3H), 2.13-2.05 (m, 2H), 2.01-1.94 (m, 2H), 1.90-1.79 (m, 3H), 1.70-1.63 (m, 2H); LCMS: C₃₀H₃₀F₅N₅O₂ requires: 587, found: m/z=588 [M+H]⁺.

### Example 672b: 2-(3-(3-((R)-fluoro(4-methyl-4H-1,2,4-triazol-3-yl)methyl)oxetan-3-yl)phenyl)-6-(((1R,3R,5S)-3-fluoro-8-azabicyclo[3.2.1]octan-8-yl)methyl)-4-(trifluoromethyl)isoindolin-1-one

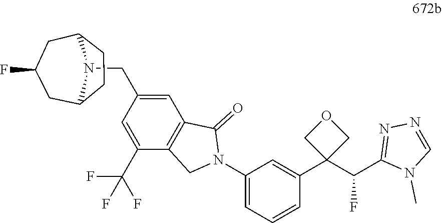

The reductive amination was carried out in a similar fashion as for 411, step 2 using (R)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (50 mg, 0.11 mmol) and (exo)-3-fluoro-8-azabicyclo[3.2.1]octane hydrochloride (52 mg, 0.32 mmol) as reactants to afford the title compound (25 mg, 40%) as a white solid. ¹H NMR (500 MHz, Methanol-d₄) δ 8.27 (s, 1H), 8.12 (s, 1H), 8.02 (s, 1H), 7.79 (ddd, J=8.3, 2.2, 0.9 Hz, 1H), 7.51 (t, J=2.0 Hz, 1H), 7.42 (t, J=8.0 Hz, 1H), 6.92 (dt, J=7.8, 1.2 Hz, 1H), 6.30 (d, J=45.3 Hz, 2H), 5.47 (d, J=6.7 Hz, 2H), 5.31 (d, J=6.2 Hz, 1H), 5.21 (dd, J=6.7, 2.0 Hz, 1H), 5.11-5.06 (m, 2H), 5.01 (dd, J=6.3, 4.1 Hz, 1H), 3.83 (s, 2H), 3.11 (d, J=0.9 Hz, 3H), 2.11-2.08 (m, 2H), 2.03-1.94 (m, 2H), 1.91-1.80 (m, 3H), 1.70-1.65 (m, 2H); LCMS: C₃₀H₃₀F₅N₅O₂ requires: 587, found: m/z=588 [M+H]⁺.

### Example 673a: (S)-2-(3-(3-(fluoro(4-methyl-4H-1,2,4-triazol-3-yl)methyl)oxetan-3-yl)phenyl)-6-(pyrrolidin-1-ylmethyl)-4-(trifluoromethyl)isoindolin-1-one

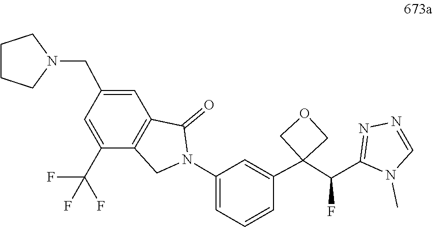 [SMILES: Cn1cnnc1[C@@H](F)C1(c2cccc(N3Cc4c(cc(CN5CCCC5)cc4C(F)(F)F)C3=O)c2)COC1]

The reductive amination was carried out in a similar fashion as for 411, step 2 using (S)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (50 mg, 0.11 mmol) and pyrrolidine (37 mg, 0.53 mmol) as reactants to afford the title compound (35 mg, 67%) as a white solid. ¹H NMR (500 MHz, Methanol-d₄) δ 8.27 (s, 1H), 8.05 (s, 1H), 7.97 (s, 1H), 7.82-7.77 (m, 1H), 7.52 (t, J=2.0 Hz, 1H), 7.42 (t, J=8.0 Hz, 1H), 6.92 (dd, J=7.8, 1.3 Hz, 1H), 6.30 (d, J=45.3 Hz, 1H), 5.47 (d, J=6.8 Hz, 1H), 5.31 (d, J=6.3 Hz, 1H), 5.21 (dd, J=6.7, 1.9 Hz, 1H), 5.14-5.05 (m, 2H), 5.01 (dd, J=6.3, 4.1 Hz, 1H), 3.84 (s, 2H), 3.11 (s, 3H), 2.63-2.54 (m, 4H), 1.89-1.79 (m, 4H); LCMS: C₂₇H₂₇F₄N₅O₂ requires: 529, found: m/z=530 [M+H]⁺.

### Example 673b: (R)-2-(3-(3-(fluoro(4-methyl-4H-1,2,4-triazol-3-yl)methyl)oxetan-3-yl)phenyl)-6-(pyrrolidin-1-ylmethyl)-4-(trifluoromethyl)isoindolin-1-one

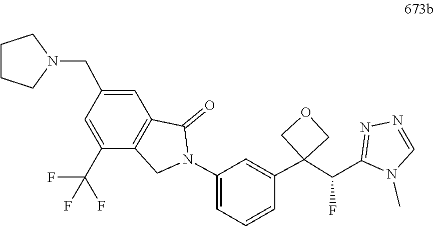 [SMILES: Cn1cnnc1[C@H](F)C1(c2cccc(N3Cc4c(cc(CN5CCCC5)cc4C(F)(F)F)C3=O)c2)COC1]

The reductive amination was carried out in a similar fashion as for 411, step 2 using (R)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (50 mg, 0.11 mmol) and pyrrolidine (37 mg, 0.53 mmol) as reactants to afford the title compound (10 mg, 17%) as a white solid. ¹H NMR (500 MHz, Methanol-d₄) δ 8.27 (s, 1H), 8.05 (s, 1H), 7.97 (s, 1H), 7.83-7.77 (m, 1H), 7.52 (t, J=2.0 Hz, 1H), 7.42 (t, J=8.0 Hz, 1H), 6.92 (dd, J=7.8, 1.3 Hz, 1H), 6.30 (d, J=45.3 Hz, 1H), 5.47 (d, J=6.8 Hz, 1H), 5.31 (d, J=6.3 Hz, 1H), 5.21 (dd, J=6.7, 1.9 Hz, 1H), 5.12-5.06 (m, 2H), 5.01 (dd, J=6.3, 4.1 Hz, 1H), 3.84 (s, 2H), 3.11 (s, 3H), 2.63-2.54 (m, 4H), 1.89-1.80 (m, 4H); LCMS: C₂₇H₂₇F₄N₅O₂ requires: 529, found: m/z=530 [M+H]⁺.

### Example 674a: (S)-2-(3-(3-(fluoro(4-methyl-4H-1,2,4-triazol-3-yl)methyl)oxetan-3-yl)phenyl)-6-(piperidin-1-ylmethyl)-4-(trifluoromethyl)isoindolin-1-one

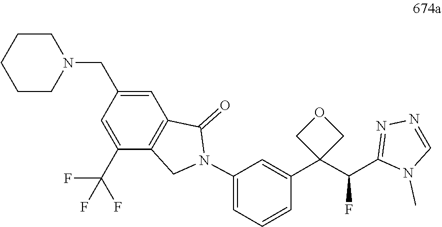 [SMILES: Cn1cnnc1[C@@H](F)C1(c2cccc(N3Cc4c(cc(CN5CCCCC5)cc4C(F)(F)F)C3=O)c2)COC1]

The reductive amination was carried out in a similar fashion as for 411, step 2 using (S)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (30 mg, 0.06 mmol) and piperidine (5 mg, 0.06 mmol) as reactants to afford the title compound (3 mg, 9%) as a white solid. ¹H NMR (500 MHz, Acetonitrile-d₃) δ 8.00 (d, J=13.5 Hz, 2H), 7.98-7.88 (m, 2H), 7.51 (t, J=2.0 Hz, 1H), 7.41 (t, J=8.0 Hz, 1H), 6.96 (dd, J=7.6, 1.4 Hz, 1H), 6.16 (d, J=46.1 Hz, 1H), 5.47-5.40 (m, 2H), 5.28 (d, J=6.3 Hz, 1H), 5.19 (dd, J=6.7, 1.9 Hz, 1H), 4.99 (s, 1H), 4.95 (dd, J=6.3, 4.3 Hz, 1H), 3.64 (s, 2H), 3.13 (d, J=0.8 Hz, 3H), 2.47-2.38 (m, 2H), 2.15-2.07 (m, 2H), 1.65-1.55 (m, 4H), 1.54-1.42 (m, 2H); LCMS: C₂₈H₂₉F₄N₅O₂ requires: 543, found: m/z=544 [M+H]⁺.

### Example 674b: (R)-2-(3-(3-(fluoro(4-methyl-4H-1,2,4-triazol-3-yl)methyl)oxetan-3-yl)phenyl)-6-(piperidin-1-ylmethyl)-4-(trifluoromethyl)isoindolin-1-one

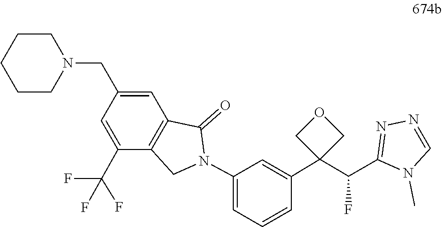 [SMILES: Cn1cnnc1[C@H](F)C1(c2cccc(N3Cc4c(cc(CN5CCCCC5)cc4C(F)(F)F)C3=O)c2)COC1]

The reductive amination was carried out in a similar fashion as for 411, step 2 using (R)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (50 mg, 0.11 mmol) and piperidine (45 mg, 0.53 mmol) as reactants to afford the title compound (35 mg, 62%) as a white solid. ¹H NMR (500 MHz, Acetonitrile-d₃) δ 8.00 (d, J=13.5 Hz, 2H), 7.97-7.89 (m, 2H), 7.51 (t, J=2.0 Hz, 1H), 7.41 (t, J=8.0 Hz, 1H), 6.96 (dd, J=7.6, 1.4 Hz, 1H), 6.16 (d, J=46.1 Hz, 1H), 5.46-5.40 (m, 2H), 5.28 (d, J=6.3 Hz, 1H), 5.19 (dd, J=6.7, 1.9 Hz, 1H), 4.99 (s, 1H), 4.95 (dd, J=6.3, 4.3 Hz, 1H), 3.64 (s, 2H), 3.13 (d, J=0.8 Hz, 3H), 2.49-2.36 (m, 2H), 2.14-2.07 (m, 2H), 1.65-1.54 (m, 4H), 1.54-1.41 (m, 2H); LCMS: C₂₈H₂₉F₄N₅O₂ requires: 543, found: m/z=544 [M+H]⁺.

### Example 675a: 6-((8-azabicyclo[3.2.1]octan-8-yl)methyl)-2-(3-(3-((S)-fluoro(4-methyl-4H-1,2,4-triazol-3-yl)methyl)oxetan-3-yl)phenyl)-4-(trifluoromethyl)isoindolin-1-one

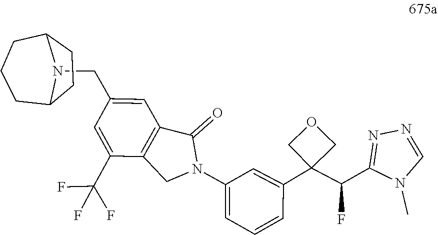 [SMILES: Cn1cnnc1[C@@H](F)C1(c2cccc(N3Cc4c(cc(CN5C6CCCC5CC6)cc4C(F)(F)F)C3=O)c2)COC1]

The reductive amination was carried out in a similar fashion as for 411, step 2 using (S)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (50 mg, 0.11 mmol) and 8-azabicyclo[3.2.1]octane hydrochloride (15 mg, 0.11 mmol) as reactants to afford the title compound (19 mg, 31%) as a white solid. ¹H NMR (500 MHz, Acetonitrile-d₃) δ 7.95 (s, 1H), 7.90 (d, J=2.6 Hz, 2H), 7.83 (ddd, J=8.2, 2.3, 0.9 Hz, 1H), 7.39 (t, J=2.0 Hz, 1H), 7.29 (t, J=8.0 Hz, 1H), 6.84 (dt, J=8.0, 1.1 Hz, 1H), 6.05 (d, J=46.1 Hz, 1H), 5.32 (dd, J=6.7, 1.1 Hz, 1H), 5.16 (dd, J=6.3, 1.0 Hz, 1H), 5.08 (dd, J=6.7, 1.9 Hz, 1H), 4.88 (s, 2H), 4.84 (dd, J=6.3, 4.2 Hz, 1H), 3.59 (s, 2H), 3.02 (d, J=4.2 Hz, 3H), 2.02-1.90 (m, 4H), 1.68-1.59 (m, 2H), 1.59-1.46 (m, 3H), 1.41-1.33 (m, 1H), 1.33-1.23 (m, 2H); LCMS: C₃₀H₃₁F₄N₅O₂ requires: 569, found: m/z=570 [M+H]⁺.

### Example 675b: 6-((8-azabicyclo[3.2.1]octan-8-yl)methyl)-2-(3-(3-((R)-fluoro(4-methyl-4H-1,2,4-triazol-3-yl)methyl)oxetan-3-yl)phenyl)-4-(trifluoromethyl)isoindolin-1-one

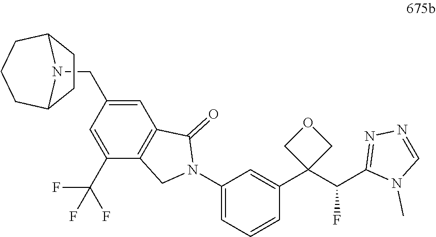 [SMILES: Cn1cnnc1[C@H](F)C1(c2cccc(N3Cc4c(cc(CN5C6CCCC5CC6)cc4C(F)(F)F)C3=O)c2)COC1]

The reductive amination was carried out in a similar fashion as for 411, step 2 using (R)-2-(3-(3-(fluoro(4-methyl-4H-1,2,4-triazol-3-yl)methyl)oxetan-3-yl)phenyl)-3-oxo-7-(trifluoromethyl)isoindoline-5-carbaldehyde (50 mg, 0.11 mmol) and 8-azabicyclo[3.2.1]octane hydrochloride (48 mg, 0.32 mmol) as reactants to afford the title compound (12 mg, 21%) as a white solid. ¹H NMR (500 MHz, Acetonitrile-d₃) δ 7.95 (s, 1H), 7.90 (d, J=2.6 Hz, 2H), 7.83 (ddd, J=8.2, 2.3, 0.9 Hz, 1H), 7.39 (t, J=2.0 Hz, 1H), 7.29 (t, J=8.0 Hz, 1H), 6.84 (dt, J=8.0, 1.1 Hz, 1H), 6.05 (d, J=46.1 Hz, 1H), 5.32 (dd, J=6.7, 1.1 Hz, 1H), 5.16 (dd, J=6.3, 1.0 Hz, 1H), 5.08 (dd, J=6.7, 1.9 Hz, 1H), 4.88 (s, 2H), 4.84 (dd, J=6.3, 4.2 Hz, 1H), 3.59 (s, 2H), 3.02 (d, J=4.2 Hz, 3H), 2.01-1.91 (m, 4H), 1.69-1.60 (m, 2H), 1.54-1.47 (m, 3H), 1.40-1.31 (m, 1H), 1.34-1.26 (m, 2H); LCMS: C₃₀H₃₁F₁N₅O₂ requires: 569, found: m/z=570 [M+H]⁺.

### Example 676: 2-cyclopropyl-N-(3-(3-(difluoro(4-methyl-4H-1,2,4-triazol-3-yl)methyl)oxetan-3-yl)phenyl)-6-methylpyrimidine-4-carboxamide

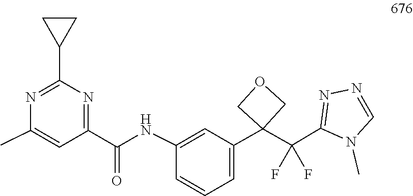 [SMILES: Cc1cc(C(=O)Nc2cccc(C3(C(F)(F)c4nncn4C)COC3)c2)nc(C2CC2)n1]

Step 1: Synthesis of 3-[3-(3-bromophenyl)oxetane-3-carbonyl]-4-methyl-4H-1,2,4-triazole. To a stirring solution of [3-(3-bromophenyl)oxetan-3-yl](4-methyl-4H-1,2,4-triazol-3-yl)methanol (130 g, 401 mmol) in DMSO (1.3 L) at 10° C. was added IBX (123 g, 441 mmol) in portions over 30 min. The solution was stirred for 3 h at 25° C. The reaction was then quenched by the addition of water/ice and then the solids were filtered off. The solution was extracted with EtOAc (3×). The organic phases were combined and concentrated. The residue was purified by flash column chromatography with EtOAc:hexanes to afford the title compound (103 g, 75%) as a white solid.

Step 2: Synthesis of 3-{[3-(3-bromophenyl)oxetan-3-yl]difluoromethyl}-4-methyl-4H-1,2,4-triazole. To a stirring solution of 3-[3-(3-bromophenyl)oxetane-3-carbonyl]-4-methyl-4H-1,2,4-triazole (75 g, 232 mmol) in DCE (225 mL) was added BAST (257 g, 1164 mmol). The reaction was then quenched by the addition of saturated sodium bicarbonate aqueous solution and extracted with DCM (3×). All the organic layers were combined, dried, filtered and concentrated under vacuum. The residue was purified by flash column chromatography with DCM/EtOAc to afford the title compound (35 g, 41%) as a white solid. ¹H NMR (300 MHz, DMSO-d₆) δ 8.55 (s, 1H), 7.53-7.56 (m, 1H), 7.29-7.36 (m, 2H), 6.35 (s, 1H), 7.16-7.19 (m, 1H), 5.23-5.27 (m, 2H), 5.00-5.03 (m, 4H), 3.29-3.32 (m, 3H); LCMS: C₁₃H₁₂BrF₂N₃O requires: 343, found: m/z=344 [M+H]⁺.

Step 3: Synthesis of 2-cyclopropyl-N-(3-(3-(difluoro(4-methyl-4H-1,2,4-triazol-3-yl)methyl)oxetan-3-yl)phenyl)-6-methylpyrimidine-4-carboxamide. The coupling reaction was carried out in a similar fashion to EXAMPLE 168, step 1 using 3-{[3-(3-bromophenyl)-oxetan-3-yl]difluoromethyl}-4-methyl-4H-1,2,4-triazole (100 mg, 0.28 mmol) and 2-cyclo-propyl-6-methylpyrimidine-4-carboxamide (56 mg, 0.32 mmol) as reactants to afford the title compound (75 mg, 59%) as an off-white solid. ¹H NMR (500 MHz, DMSO-d₆) δ 10.50 (s, 1H), 8.51 (s, 1H), 7.97-7.78 (m, 1H), 7.72 (s, 1H), 7.62 (t, J=2.0 Hz, 1H), 7.36 (t, J=8.0 Hz, 1H), 6.82 (d, J=7.7 Hz, 1H), 5.31 (d, J=6.8 Hz, 2H), 5.02 (dd, J=7.0, 2.1 Hz, 2H), 3.15 (d, J=1.7 Hz, 3H), 2.53 (s, 3H), 2.39-2.31 (m, 1H), 1.23-1.06 (m, 4H); LCMS: C₂₂H₂₂F₂N₆O₂ requires: 440, found: m/z=441 [M+H]⁺.

### Example 677: 4-cyclopropyl-2-(3-(3-(difluoro(4-methyl-4H-1,2,4-triazol-3-yl)methyl)oxetan-3-yl)phenyl)-6-methyl-2,3-dihydro-1H-pyrrolo[3,4-c]pyridin-1-one

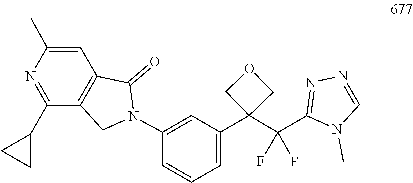 [SMILES: Cc1cc2c(c(C3CC3)n1)CN(c1cccc(C3(C(F)(F)c4nncn4C)COC3)c1)C2=O]

The coupling reaction was carried out in a similar fashion to EXAMPLE 168, step 1 using 3-{[3-(3-bromophenyl)oxetan-3-yl]difluoromethyl}-4-methyl-4H-1,2,4-triazole (90 mg, 0.26 mmol) and 4-cyclopropyl-6-methyl-1H, 2H,3H-pyrrolo[3,4-c]pyridin-1-one (49 mg, 0.26 mmol) as reactants to afford the title compound (53 mg, 45%) as an off-white solid. ¹H NMR (500 MHz, DMSO-d₆) δ 8.43 (s, 1H), 7.92 (dd, J=8.1, 2.1 Hz, 1H), 7.49 (d, J=2.0 Hz, 1H), 7.36 (t, J=8.0 Hz, 1H), 7.26 (s, 1H), 6.85 (d, J=8.1 Hz, 1H), 5.25 (d, J=6.9 Hz, 2H), 5.05-4.92 (m, 4H), 3.25 (s, 3H), 3.05 (s, 3H), 2.10 (tt, J=7.8, 4.8 Hz, 1H), 1.08-0.92 (m, 4H); LCMS: C₂₄H₂₃F₂N₅O₂ requires: 451, found: m/z=452 [M+H]⁺.

### Example 678: 2-(3-(3-(difluoro(4-methyl-4H-1,2,4-triazol-3-yl)methyl)oxetan-3-yl)phenyl)-6-(l-((S)-3-fluoropyrrolidin-1-yl)ethyl)-4-(trifluoromethyl)isoindolin-1-one

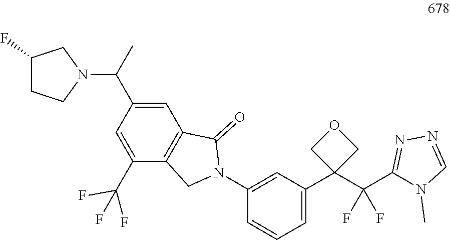 [SMILES: CC(c1cc2c(c(C(F)(F)F)c1)CN(c1cccc(C3(C(F)(F)c4nncn4C)COC3)c1)C2=O)N1CC[C@H](F)C1]

Step 1: Synthesis of 6-acetyl-4-(trifluoromethyl)-2,3-dihydro-1H-isoindol-1-one. A solution of methyl 5-acetyl-2-(bromomethyl)-3-(trifluoromethyl)benzoate (2.0 g, 5.9 mmol) in methanol (50 mL) was added ammonia (1.3 mL, 7N in Methanol). The reaction mixture was stirred at rt for 12 h and evaporated to dryness. The solution was partitioned between EtOAc and water. The layers were dried, filtered, and concentrated. The solid was purified by chromatography to yield the title compound (1.0 g, 70%) as a white solid.

Step 2: Synthesis of 6-{l-[(3S)-3-fluoropyrrolidin-1-yl]ethyl}-4-(trifluoromethyl)-2,3-dihydro-1H-isoindol-1-one. Reductive animation of the methyl ketone was carried out according to the procedure for 459, step 2 using 6-acetyl-4-(trifluoromethyl)-2,3-dihydro-1H-isoindol-1-one (700 mg, 2.88 mmol) and (3S)-3-fluoropyrrolidine hydrochloride (723 mg, 5.76 mmol) as reactants to afford the title compound (680 mg, 75%).

Step 3: Synthesis of 2-(3-(3-(difluoro(4-methyl-4H-1,2,4-triazol-3-yl)methyl)-oxetan-3-yl)phenyl)-6-(l-((S)-3-fluoropyrrolidin-1-yl)ethyl)-4-(trifluoromethyl)isoindolin-1-one. The coupling reaction was carried out in a similar fashion to EXAMPLE 168, step 1 using 3-{[3-(3-bromophenyl)oxetan-3-yl]difluoromethyl}-4-methyl-4H-1,2,4-triazole (80 mg, 0.23 mmol) and 6-{l-[(3S)-3-fluoropyrrolidin-1-yl]ethyl}-4-(trifluoromethyl)-2,3-dihydro-1H-isoindol-1-one (73 mg, 0.23 mmol) as reactants to afford the title compound (19 mg, 14%) as an off-white solid. ¹H NMR (500 MHz, DMSO-d₆) δ 8.41 (s, 1H), 7.94 (s, 1H), 7.92-7.81 (m, 2H), 7.44 (q, J=2.2 Hz, 1H), 7.37 (t, J=8.0 Hz, 1H), 6.87 (d, J=7.8 Hz, 1H), 5.25 (d, J=7.0 Hz, 2H), 5.21-5.08 (m, 1H), 5.02 (s, 2H), 4.97 (d, J=7.0 Hz, 2H), 3.55 (dq, J=13.3, 6.6 Hz, 1H), 3.03 (s, 3H), 2.90-2.77 (m, 1H), 2.37-2.27 (m, 1H), 2.23-1.97 (m, 2H), 1.91-1.73 (m, 2H), 1.30 (d, J=6.5, 2.2 Hz, 3H); LCMS: C₂₈H₂₇F₆N₅O₂ requires: 579, found: m/z=580 [M+H]⁺.

### Example 679: 2-(3-(3-(difluoro(4-methyl-4H-1,2,4-triazol-3-yl)methyl)oxetan-3-yl)phenyl)-6-((3-fluoroazetidin-1-yl)methyl)-4-(trifluoromethyl)isoindolin-1-one

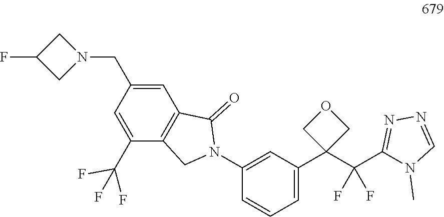 [SMILES: Cn1cnnc1C(F)(F)C1(c2cccc(N3Cc4c(cc(CN5CC(F)C5)cc4C(F)(F)F)C3=O)c2)COC1]

Step 1: Synthesis of methyl 2-(bromomethyl)-5-[(3-fluoroazetidin-1-yl)methyl]-3-(trifluoromethyl)benzoate. The reductive animation was carried out in a similar fashion as for 411, step 2 using ethyl 2-(bromomethyl)-5-formyl-3-(trifluoromethyl)benzoate (700 mg, 2.15 mmol) and 3-fluoroazetidine hydrochloride (240 mg, 2.15 mmol) as reactants to afford the title compound (250 mg, 30%) as a white solid.

Step 2: Synthesis of 6-[(3-fluoroazetidin-1-yl)methyl]-4-(trifluoromethyl)-2,3-dihydro-1H-isoindol-1-one. A solution of methyl 2-(bromomethyl)-5-[(3-fluoroazetidin-1-yl)-methyl]-3-(trifluoromethyl)benzoate (225 mg, 0.59 mmol) was added ammonia (7N in MeOH, 2 mL). The reaction mixture was stirred at rt for 1 h and evaporated to dryness. The solution was partitioned between EtOAc and water. The layers were dried, filtered, and concentrated. The crude material was purified by chromatography to give the title compound (160 mg, 95%).

Step 3: Synthesis of 2-(3-(3-(difluoro(4-methyl-4H-1,2,4-triazol-3-yl)methyl)-oxetan-3-yl)phenyl)-6-((3-fluoroazetidin-1-yl)methyl)-4-(trifluoromethyl)isoindolin-1-one. The coupling reaction was carried out in a similar fashion to Example 168, step 1 using 3-{[3-(3-bromophenyl)oxetan-3-yl]difluoromethyl}-4-methyl-4H-1,2,4-triazole (50 mg, 0.15 mmol) and 6-[(3-fluoroazetidin-1-yl)methyl]-4-(trifluoromethyl)-2,3-dihydro-1H-isoindol-1-one (42 mg, 0.15 mmol) as reactants to afford the title compound (25 mg, 31%) as an off-white solid. ¹H NMR (500 MHz, DMSO-d₆) δ 8.48 (s, 1H), 7.97 (s, 1H), 7.93 (td, J=4.3, 3.5, 1.4 Hz, 2H), 7.54 (t, J=1.9 Hz, 1H), 7.44 (t, J=8.0 Hz, 1H), 6.94 (d, J=7.8 Hz, 1H), 5.33 (d, J=6.9 Hz, 2H), 5.28 (ddd, J=10.2, 6.2, 4.2 Hz, 1H), 5.20-5.14 (m, 1H), 5.10 (s, 1H), 5.05 (dd, J=7.0, 2.1 Hz, 2H), 3.86 (s, 2H), 3.65-3.55 (m, 2H), 3.26-3.17 (m, 2H), 3.11 (s, 3H); LCMS: C₂₆H₂₃F₆N₅O₂ requires: 551, found: m/z=552 [M+H]⁺.

### Example 680: (R)-2-(6-cyclopropyl-4-(1-(4-methyl-4H-1,2,4-triazol-3-yl)propan-2-yl)pyridin-2-yl)-4-(trifluoromethyl)isoindolin-1-one

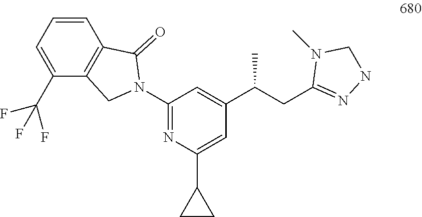 [SMILES: C[C@H](CC1=NNCN1C)c1cc(C2CC2)nc(N2Cc3c(cccc3C(F)(F)F)C2=O)c1]

To a solution of 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (250 mg, 0.57 mmol), 2-cyclopropyl-4,4,5,5-tetramethyl-1,3,2-dioxaborolane (289.17 mg, 1.72 mmol), and 1,1′-Bis(diphenylphosphino)ferrocene-palladium(II)dichloride DCM complex (0.05 g, 0.06 mmol) in 1,4-dioxane (6 mL) and water (0.5 mL) was added cesium carbonate (0.56 g, 1.72 mmol). The solution was heated at 100° C. for 12 h. The solution was filtered through celite and purified by HPLC to afford the title compound (65 mg, 23%) as an off-white solid, 1H NMR (500 MHz, DMSO-d₆) δ 8.32 (d, J=3.4 Hz, 1H), 8.21 (d, J=1.3 Hz, 1H), 8.10 (dt, J=23.5, 8.6 Hz, 2H), 7.82 (q, J=9.0, 7.8 Hz, 1H), 7.11 (d, J=1.3 Hz, 1H), 5.21 (s, 2H), 3.58 (d, J=10.3 Hz, 3H), 3.19-2.89 (m, 3H), 2.13-2.05 (m, 1H), 1.40-1.23 (m, 3H), 1.11-0.85 (m, 4H); LCMS: C₂₂H₂₂F₃N₆O requires: 441, found: m/z=442 [M+H]⁺.

### Example 681: (R)-2-(4-(1-(4-methyl-4H-1,2,4-triazol-3-yl)propan-2-yl)-6-(l-methylcyclopropyl)pyridin-2-yl)-4-(trifluoromethyl)isoindolin-1-one

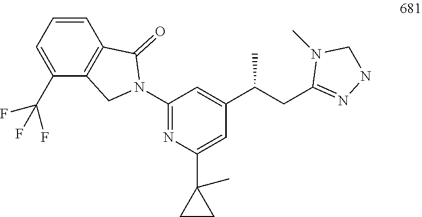 [SMILES: C[C@H](CC1=NNCN1C)c1cc(N2Cc3c(cccc3C(F)(F)F)C2=O)nc(C2(C)CC2)c1]

To a stirring solution of 2-{6-chloro-4-[(2R)-1-(4-methyl-1,2,4-triazol-3-yl)propan-2-yl]pyridin-2-yl}-4-(trifluoromethyl)-3H-isoindol-1-one (100 mg, 0.23 mmol) and 4,4,5,5-tetra-methyl-2-(l-methylcyclopropyl)-1,3,2-dioxaborolane (83 mg, 0.46 mmol) in 1,4-dioxane (2.00 mL) and water (0.50 mL) was added tetrakis(triphenylphosphine) palladium (39 mg, 0.03 mmol) and potassium carbonate (63 mg, 0.46 mmol). The solution was heated at 100° C. for 12 h. The solution was filtered through celite and purified by HPLC. The compound was neutralized (PL-HCO3 MP SPE) to give the title compound (5.2 mg, 5.0%). ¹H NMR (500 MHz, Acetonitrile-d₃) δ 8.18 (d, J=1.3 Hz, 1H), 7.99 (d, J=7.6 Hz, 1H), 7.92 (s, 1H), 7.86 (dt, J=7.6, 0.9 Hz, 1H), 7.64 (tq, J=7.8, 0.9 Hz, 1H), 6.94 (d, J=1.3 Hz, 1H), 5.11 (d, J=1.6 Hz, 2H), 3.41 (s, 3H), 3.04-2.90 (m, 2H), 2.05 (s, 3H), 1.31 (d, J=7.0 Hz, 3H), 1.18 (q, J=3.1 Hz, 2H), 0.78-0.70 (m, 2H); LCMS: C₂₄H₂₄F₃N₅O requires: 455, found: m/z=456 [M+H]⁺.

### Example 682: (R)-2-(4-(1-(4-methyl-4H-1,2,4-triazol-3-yl)propan-2-yl)-6-(thiazol-5-yl)pyridin-2-yl)-4-(trifluoromethyl)isoindolin-1-one

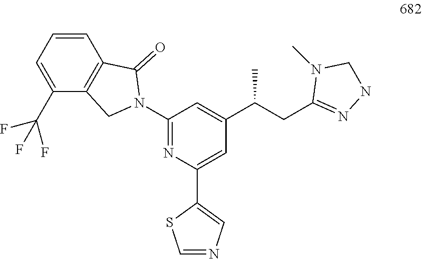 [SMILES: C[C@H](CC1=NNCN1C)c1cc(-c2cncs2)nc(N2Cc3c(cccc3C(F)(F)F)C2=O)c1]

The coupling reaction was carried out according to Example 680 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (50 mg, 0.11 mmol) and 5-(4,4,5,5-tetramethyl-1,3,2-dioxaborolan-2-yl)-1,3-thiazole (73 mg, 0.34 mmol) as reactants to afford the title compound (8 mg, 14%) as an off-white solid. ¹H NMR (500 MHz, Acetonitrile-d₃) δ 8.83 (s, 1H), 8.37 (dd, J=5.2, 0.9 Hz, 2H), 8.01 (d, J=7.6 Hz, 1H), 7.94-7.86 (m, 2H), 7.69-7.62 (m, 1H), 7.49 (d, J=1.3 Hz, 1H), 5.19 (s, 2H), 3.44 (s, 3H), 3.43-3.37 (m, 1H), 3.10-2.95 (m, 2H), 1.35 (d, J=6.9 Hz, 3H); LCMS: C₂₃H₁₉F₃N₆OS requires: 484, found: m/z=485 [M+H]⁺.

### Example 683: ((R)-2-(6-(4-methyl-1H-pyrazol-3-yl)-4-(1-(4-methyl-4H-1,2,4-triazol-3-yl)propan-2-yl)pyridin-2-yl)-4-(trifluoromethyl)isoindolin-1-one

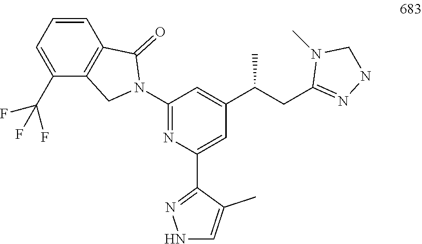 [SMILES: Cc1c[nH]nc1-c1cc([C@H](C)CC2=NNCN2C)cc(N2Cc3c(cccc3C(F)(F)F)C2=O)n1]

The coupling reaction was carried out according to Example 680 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (50 mg, 0.11 mmol) and 4-methyl-3-(4,4,5,5-tetramethyl-1,3,2-dioxaborolan-2-yl)-2H-pyrazole (70 mg, 0.34 mmol) as reactants to afford the title compound (53 mg, 96%) as an off-white solid. ¹H NMR (500 MHz, Acetonitrile-d₃) δ 8.37 (s, 1H), 8.02 (d, J=7.6 Hz, 1H), 7.93-7.86 (m, 2H), 7.69-7.62 (m, 1H), 7.38 (s, 1H), 5.27 (s, 2H), 3.43 (s, 3H), 3.08-2.95 (m, 2H), 2.42 (s, 1H), 2.35 (s, 2H), 2.06 (s, 3H), 1.35 (d, J=6.9 Hz, 3H); LCMS: C₂₄H₂₂F₃N₇O requires: 481, found: m/z=482 [M+H]⁺.

### Example 684: (R)-2-(4-(1-(4-methyl-4H-1,2,4-triazol-3-yl)propan-2-yl)-6-(1H-pyrazol-4-yl)pyridin-2-yl)-4-(trifluoromethyl)isoindolin-1-one

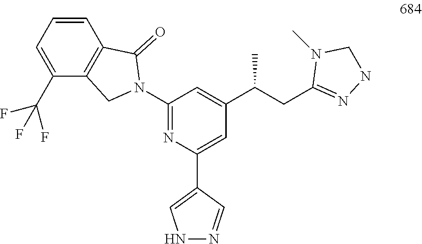 [SMILES: C[C@H](CC1=NNCN1C)c1cc(-c2cn[nH]c2)nc(N2Cc3c(cccc3C(F)(F)F)C2=O)c1]

The coupling reaction was carried out according to Example 680 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (50 mg, 0.11 mmol) and 1H-pyrazol-4-ylboronic acid hydrochloride (50 mg, 0.34 mmol) as reactants to afford the title compound (26 mg, 49%) as an off-white solid. ¹H NMR (500 MHz, Acetonitrile-d₃) δ 8.26 (d, J=1.3 Hz, 1H), 8.07-7.98 (m, 2H), 7.94-7.85 (m, 2H), 7.69-7.62 (m, 1H), 7.25 (d, J=1.3 Hz, 1H), 5.24 (s, 2H), 3.43 (s, 3H), 3.40-3.31 (m, 2H), 3.08-2.94 (m, 3H), 1.34 (d, J=7.0 Hz, 3H); LCMS: C₂₃H₂₀F₃N₇O requires: 467, found: m/z=468 [M+H]⁺.

### Example 685: (R)-2-(6-(3,6-dihydro-2H-pyran-4-yl)-4-(1-(4-methyl-4H-1,2,4-triazol-3-yl)propan-2-yl)pyridin-2-yl)-4-(trifluoromethyl)isoindolin-1-one

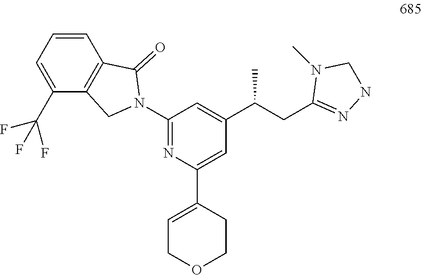 [SMILES: C[C@H](CC1=NNCN1C)c1cc(C2=CCOCC2)nc(N2Cc3c(cccc3C(F)(F)F)C2=O)c1]

The coupling reaction was carried out according to Example 680 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (50 mg, 0.11 mmol) and 2-(3,6-dihydro-2H-pyran-4-yl)-4,4,5,5-tetramethyl-1,3,2-dioxaborolane (70 mg, 0.34 mmol) as reactants to afford the title compound (17 mg, 32%) as an off-white solid. ¹H NMR (500 MHz, Acetonitrile-d₃) δ 8.43 (d, J=1.4 Hz, 1H), 8.11 (d, J=7.6 Hz, 1H), 8.04-7.95 (m, 2H), 7.79-7.72 (m, 1H), 7.20 (d, J=1.3 Hz, 1H), 6.85 (tt, J=3.0, 1.6 Hz, 1H), 5.29 (s, 2H), 4.35 (q, J=2.9 Hz, 2H), 3.92 (t, J=5.5 Hz, 2H), 3.52 (s, 4H), 3.16-3.03 (m, 2H), 2.63 (td, J=5.7, 2.6 Hz, 2H), 1.43 (d, J=7.0 Hz, 3H); LCMS: C₂₅H₂₄F₃N₅O₂ requires: 483, found: m/z=484 [M+H]⁺.

### Example 686: (R)-2-(4-(1-(4-methyl-4H-1,2,4-triazol-3-yl)propan-2-yl)-6-(tetrahydro-2H-pyran-4-yl)pyridin-2-yl)-4-(trifluoromethyl)isoindolin-1-one

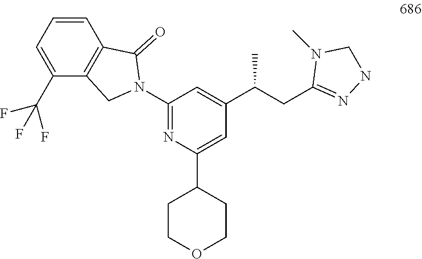 [SMILES: C[C@H](CC1=NNCN1C)c1cc(C2CCOCC2)nc(N2Cc3c(cccc3C(F)(F)F)C2=O)c1]

A solution of 2-[6-(3,6-dihydro-2H-pyran-4-yl)-4-[(2R)-1-(4-methyl-1,2,4-triazol-3-yl)propan-2-yl]pyridin-2-yl]-4-(trifluoromethyl)-3H-isoindol-1-one (16 mg, 0.03 mmol) and palladium (20.00 mg, 10 wt % on carbon) in acetic acid (0.10 mL) and methanol (0.5 mL) was stirred at rt for 2 hours under an atmosphere of hydrogen. The residue was filtered through celite, concentrated and then purified by HPLC. The compound was neutralized (PL-HCO3 MP SPE) to afford the title compound (5 mg, 30%) as a white solid. ¹H NMR (500 MHz, Methanol-d₄) δ 8.27-8.20 (m, 2H), 8.05 (d, J=7.6 Hz, 1H), 7.92 (d, J=7.7 Hz, 1H), 7.71 (t, J=7.7 Hz, 1H), 6.90 (d, J=1.2 Hz, 1H), 5.29-5.17 (m, 2H), 4.06-3.98 (m, 2H), 3.59-3.44 (m, 6H), 3.36 (p, J=7.2 Hz, 1H), 3.17-3.04 (m, 2H), 2.91 (tt, J=11.7, 4.0 Hz, 1H), 1.94-1.81 (m, 2H), 1.81-1.74 (m, 2H), 1.39 (d, J=6.9 Hz, 3H); LCMS: C₂₅H₂₆F₃N₅O₂ requires: 485, found: m/z=486 [M+H]⁺.

### Example 687: (R)-2-(6-(3,6-dihydro-2H-pyran-4-yl)-4-(1-(4-methyl-4H-1,2,4-triazol-3-yl)propan-2-yl)pyridin-2-yl)-4-(trifluoromethyl)isoindolin-1-one

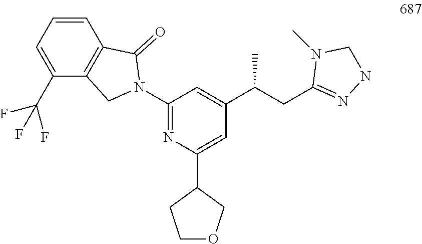 [SMILES: C[C@H](CC1=NNCN1C)c1cc(C2CCOC2)nc(N2Cc3c(cccc3C(F)(F)F)C2=O)c1]

Step 1: Synthesis of 2-[6-(2,5-dihydrofuran-3-yl)-4-[(2R)-1-(4-methyl-1,2,4-triazol-3-yl)propan-2-yl]pyridin-2-yl]-4-(trifluoromethyl)-3H-isoindol-1-one. The coupling reaction was carried out according to Example 680 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (50 mg, 0.11 mmol) and 2-(3,6-dihydro-2H-pyran-4-yl)-4,4,5,5-tetramethyl-1,3,2-dioxaborolane (70 mg, 0.34 mmol) as reactants to afford the title compound (9 mg, 17%) as an off-white solid.

Step 2: Synthesis of (R)-2-(6-(3,6-dihydro-2H-pyran-4-yl)-4-(1-(4-methyl-4H-1,2,4-triazol-3-yl)propan-2-yl)pyridin-2-yl)-4-(trifluoromethyl)isoindolin-1-one. A solution of 2-[6-(2,5-dihydrofuran-3-yl)-4-[(2R)-1-(4-methyl-1,2,4-triazol-3-yl)propan-2-yl]pyridin-2-yl]-4-(trifluoromethyl)-3H-isoindol-1-one (9 mg, 0.02 mmol) and palladium (11 mg, 0.11 mmol, 10 wt % on carbon) in methanol (1 mL) and acetic acid (0.1 mL) was stirred at rt for 2 hours under an atmosphere of hydrogen. The residue was filtered through celite, concentrated, and then purified by HPLC. The compound was neutralized (PL-HCO3 MP SPE) to afford the title compound (3 mg, 30%) as a white solid. ¹H NMR (500 MHz, Methanol-d₄) δ 8.32-8.27 (m, 3H), 8.10 (d, J=7.6 Hz, 1H), 7.97 (dt, J=7.8, 0.8 Hz, 1H), 7.79-7.72 (m, 1H), 6.99 (t, J=1.4 Hz, 1H), 5.27 (s, 2H), 4.15 (td, J=8.0, 5.4 Hz, 1H), 4.11-4.03 (m, 1H), 3.99-3.85 (m, 2H), 3.60 (s, 3H), 3.40 (p, J=7.2 Hz, 1H), 3.34 (s, 1H), 3.14 (d, J=1.8 Hz, 1H), 2.39-2.19 (m, 2H), 1.43 (d, J=7.0 Hz, 3H)); LCMS: C₂₄H₂₄F₃N₅O₂ requires: 471, found: m/z=472 [M+H]⁺.

### Example 688: (R)-2-(4-(1-(4-methyl-4H-1,2,4-triazol-3-yl)propan-2-yl)-6-propoxy-pyridin-2-yl)-4-(trifluoromethyl)isoindolin-1-one

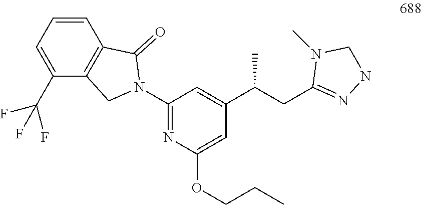 [SMILES: CCCOc1cc([C@H](C)CC2=NNCN2C)cc(N2Cc3c(cccc3C(F)(F)F)C2=O)n1]

The coupling reaction was carried out according to General Procedure 8 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (250 mg, 0.57 mmol) and propanol (0.13 mL, 1.74 mmol) as reactants to afford the title compound (43 mg, 16%) as an off-white solid. ¹H NMR (500 MHz, DMSO-d₆) δ 8.32 (s, 1H), 8.12 (d, J=7.6 Hz, 1H), 8.07 (d, J=7.7 Hz, 1H), 8.04 (d, J=1.2 Hz, 1H), 7.81 (t, J=7.7 Hz, 1H), 6.60 (s, 2H), 5.24 (s, 2H), 4.25 (t, J=6.7 Hz, 2H), 3.58 (d, J=1.2 Hz, 3H), 3.12-2.92 (m, 2H), 1.78 (h, J=7.2 Hz, 2H), 1.30 (d, J=6.9 Hz, 3H), 1.00 (t, J=7.4 Hz, 3H); LCMS: requires: 459, found: m/z=460 [M+H]⁺.

### Example 689a and 689b: 2-(6-(((1R,2S)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one and 2-(6-(((1S,2R)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one

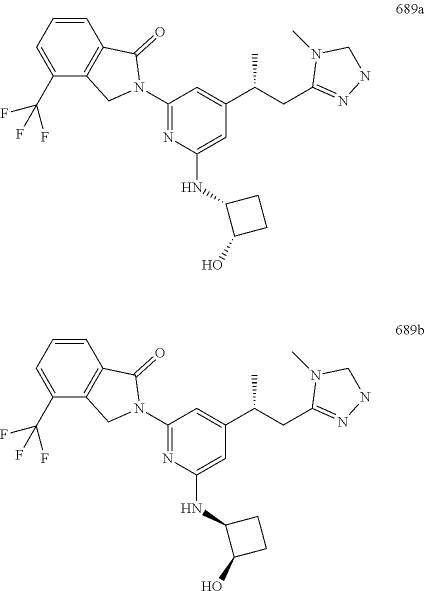 [SMILES: C[C@H](CC1=NNCN1C)c1cc(N[C@@H]2CC[C@@H]2O)nc(N2Cc3c(cccc3C(F)(F)F)C2=O)c1 ; C[C@H](CC1=NNCN1C)c1cc(N[C@H]2CC[C@H]2O)nc(N2Cc3c(cccc3C(F)(F)F)C2=O)c1]

Step 1 (XX): Synthesis of 2-(6-(((cis)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one. The amide bond formation reaction was carried out in a similar fashion as for 184 using 2-{6-chloro-4-[(2R)-1-(4-methyl-4H-1,2,4-triazol-3-yl)propan-2-yl]pyridin-2-yl}-4-(trifluoromethyl)-2,3-dihydro-1H-isoindol-1-one (200 mg, 0.46 mmol) and (cis)-2-aminocyclobutan-1-ol hydrochloride (113 mg, 0.92 mmol) as reactants afforded the title compound (48 mg, 21%) as an off-white solid.

Step 2: 2-(6-(((1R,2S)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one and 2-(6-(((1S,2R)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one. 2-(6-(((cis)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one was separated using IG column with CO₂ and methanol as mobile phase to afford 2-(6-(((1R,2S)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one (11 mg) and 2-(6-(((1S,2R)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one (12 mg).

2-(6-(((1R,2S)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one. ¹H NMR (500 MHz, Acetonitrile-d₃) δ 7.97 (dd, J=7.9, 1.8 Hz, 1H), 7.91 (d, J=3.9 Hz, 1H), 7.85 (dq, J=7.8, 0.9 Hz, 1H), 7.69-7.60 (m, 2H), 6.08 (s, 2H), 5.38 (t, J=5.6 Hz, 1H), 5.09 (d, J=2.5 Hz, 2H), 4.41-4.34 (m, 1H), 4.20-4.14 (m, 1H), 3.42 (d, J=4.5 Hz, 3H), 3.21-3.14 (m, 1H), 3.00-2.86 (m, 2H), 2.14-2.08 (m, 2H), 1.84-1.76 (m, 2H), 1.26 (dd, J=7.0, 1.1 Hz, 3H); LCMS: C₂₄H₂₅F₃N₆O2 requires: 486, found: m/z=487 [M+H]⁺.

2-(6-(((1S,2R)-2-hydroxycyclobutyl)amino)-4-((R)-1-(4-methyl-4H-1,2,4-triazol-3-yl)propan-2-yl)pyridin-2-yl)-4-(trifluoromethyl)isoindolin-1-one. ¹H NMR (500 MHz, Acetonitrile-d₃) δ 7.97 (dd, J=7.9, 1.8 Hz, 1H), 7.91 (d, J=3.9 Hz, 1H), 7.85 (dq, J=7.8, 0.9 Hz, 1H), 7.69-7.62 (m, 2H), 6.08 (s, 2H), 5.38 (t, J=5.6 Hz, 1H), 5.09 (d, J=2.5 Hz, 2H), 4.45-4.34 (m, 1H), 4.21-4.17 (m, 1H), 3.42 (d, J=4.5 Hz, 3H), 3.20-3.14 (m, 1H), 3.02-2.86 (m, 2H), 2.14-2.10 (m, 2H), 1.86-1.74 (m, 2H), 1.26 (dd, J=7.0, 1.1 Hz, 3H); LCMS: C₂₄H₂₅F₃N₆O₂ requires: 486, found: m/z=487 [M+H]⁺.

### Example 690: 2-cyclopropyl-N-(3-ethoxy-5-(3-((4-methyl-4H-1,2,4-triazol-3-yl)methyl)oxetan-3-yl)phenyl)-6-methylpyrimidine-4-carboxamide

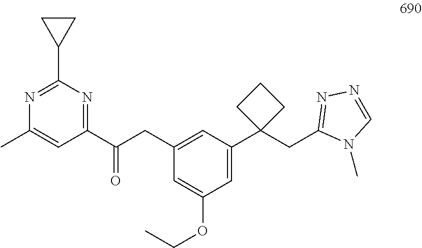 [SMILES: CCOc1cc(CC(=O)c2cc(C)nc(C3CC3)n2)cc(C2(Cc3nncn3C)CCC2)c1]

Step 1: Synthesis of ethyl 2-[3-(3-bromo-5-ethoxyphenyl)oxetan-3-yl]acetate. To a stirring solution of bis((1Z,5Z)-cycloocta-1,5-diene); bis(chlororhodium) (173 mg, 0.35 mmol) and ethyl 2-(oxetan-3-ylidene)acetate (1.00 g, 7.03 mmol) in dioxane (40 mL) was added potassium hydroxide (0.79 g, 14.07 mmol) in water (0.6 mL). After 5 min, (3-bromo-5-ethoxyphenyl)boronic acid (1.72 g, 7.03 mmol) was added. The reaction was heated at 40° C. for about 18 h under nitrogen, cooled to rt, and then concentrated. The reaction was diluted with water and the aqueous layer was extracted with EtOAc. The combined organic layers were washed, dried, filtered, and concentrated in vacuo. The crude was purified by chromatography to yield the title compound (1.40 g, 58%).

Step 2: Synthesis of 2-[3-(3-bromo-5-ethoxyphenyl)oxetan-3-yl]acetohydrazide. To a stirring solution of ethyl 2-[3-(3-bromo-5-ethoxyphenyl)oxetan-3-yl]acetate (1.35 g, 3.95 mmol) in ethanol (40 mL) was added hydrazine hydrate (3.88 mL 39.50 mmol). The mixture was stirred at 80° C. for about 12 h, cooled to rt, and then concentrated. The reaction was diluted with water and extracted with EtOAc. The combined organic layers were washed, dried, filtered, and concentrated under reduced pressure. The crude was used in the next step.

Step 3: Synthesis of 5-{[3-(3-bromo-5-ethoxyphenyl)oxetan-3-yl]methyl}-4-methyl-4H-1,2,4-triazole-3-thiol. To a solution of 2-[3-(3-bromo-5-ethoxyphenyl)oxetan-3-yl]acetohydrazide (1.30 g, 3.95 mmol) in THF (40 mL) was added isothiocyanatomethane (0.39 mL, 5.92 mmol). The mixture was stirred at rt for 4 h. KOH (1M, 30 mL) was added and the reaction was stirred overnight. The solution was partitioned between DCM and water. All the organic layers were combined, dried, filtered and concentrated under vacuum. The residue was purified by flash column chromatography with DCM/EtOAc to afford the title compound (1.10 g, 73%) as a clear oil.

Step 4: Synthesis of 3-{[3-(3-bromo-5-ethoxyphenyl)oxetan-3-yl]methyl}-4-methyl-4H-1,2,4-triazole. To solution of 5-{[3-(3-bromo-5-ethoxyphenyl)oxetan-3-yl]methyl}-4-methyl-4H-1,2,4-triazole-3-thiol (1.10 g, 2.86 mmol) in DCM (5 mL) and acetic acid (2.5 mL) was treated with hydrogen peroxide (30% aqueous solution, 1 mL) dropwise. The reaction mixture was stirred at rt for 16 h and evaporated to dryness. The solution was partitioned between DCM and sodium bicarbonate. All the organic layers were combined, dried, filtered and concentrated under vacuum. The residue was purified by flash column chromatography with DCM/EtOAc to afford the title compound. (820 mg, 81%).

Step 5: Synthesis of 2-cyclopropyl-N-(3-ethoxy-5-(3-((4-methyl-4H-1,2,4-triazol-3-yl)methyl)oxetan-3-yl)phenyl)-6-methylpyrimidine-4-carboxamide. The coupling reaction was carried out in a similar fashion to Example 168, step 1 using 3-{[3-(3-bromo-5-ethoxy-phenyl)oxetan-3-yl]methyl}-4-methyl-4H-1,2,4-triazole (70 mg, 0.20 mmol) and 2-cyclopropyl-6-methylpyrimidine-4-carboxamide (35 mg, 0.20 mmol) as reactants to afford the title compound (37 mg, 42%) as an off-white solid. ¹H NMR (500 MHz, DMSO-d₆) δ 10.35 (s, 1H), 8.23 (s, 1H), 7.71 (s, 1H), 7.47 (t, J=2.1 Hz, 1H), 7.07 (t, J=1.6 Hz, 1H), 6.30-6.17 (m, 1H), 4.88 (dd, J=33.0, 6.0 Hz, 4H), 3.94 (q, J=7.0 Hz, 2H), 3.49 (s, 2H), 2.98 (s, 3H), 2.52 (s, 3H), 2.34 (tt, J=8.1, 4.7 Hz, 1H), 1.31 (t, J=6.9 Hz, 3H), 1.26-1.04 (m, 4H); LCMS: C₂₄H₂₈N₆O₃ requires: 448, found: m/z=449 [M+H]⁺.

### Example 691a and 691b: 2-(3-ethoxy-5-(3-((4-methyl-4H-1,2,4-triazol-3-yl)methyl)-oxetan-3-yl)phenyl)-6-((R)-1-((S)-3-fluoropyrrolidin-1-yl)ethyl)-4-(trifluoromethyl)-isoindolin-1-one and 2-(3-ethoxy-5-(3-((4-methyl-4H-1,2,4-triazol-3-yl)methyl)oxetan-3-yl)phenyl)-6-((S)-1-((S)-3-fluoropyrrolidin-1-yl)ethyl)-4-(trifluoromethyl)isoindolin-1-one

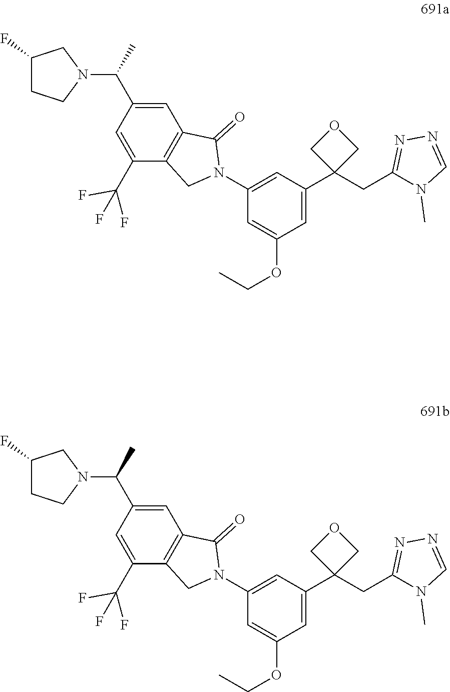 [SMILES: CCOc1cc(N2Cc3c(cc([C@@H](C)N4CC[C@H](F)C4)cc3C(F)(F)F)C2=O)cc(C2(Cc3nncn3C)COC2)c1 ; CCOc1cc(N2Cc3c(cc([C@H](C)N4CC[C@H](F)C4)cc3C(F)(F)F)C2=O)cc(C2(Cc3nncn3C)COC2)c1]

Step 1: Synthesis of 2-(3-ethoxy-5-(3-((4-methyl-4H-1,2,4-triazol-3-yl)methyl)-oxetan-3-yl)phenyl)-6-(l-((S)-3-fluoropyrrolidin-1-yl)ethyl)-4-(trifluoromethyl)isoindolin-1-one. The coupling reaction was carried out in a similar fashion to Example 168, step 1 using 3-{[3-(3-bromo-5-ethoxyphenyl)oxetan-3-yl]methyl}-4-methyl-4H-1,2,4-triazole (70 mg, 0.20 mmol) and 6-{l-[(3S)-3-fluoropyrrolidin-1-yl]ethyl}-4-(trifluoromethyl)-2,3-dihydro-1H-isoindol-1-one (63 mg, 0.20 mmol) as reactants to afford the title compound (30 mg, 26%).

Step 2: 2-(3-ethoxy-5-(3-((4-methyl-4H-1,2,4-triazol-3-yl)methyl)oxetan-3-yl)phenyl)-6-((R)-1-((S)-3-fluoropyrrolidin-1-yl)ethyl)-4-(trifluoromethyl)isoindolin-1-one and 2-(3-ethoxy-5-(3-((4-methyl-4H-1,2,4-triazol-3-yl)methyl)oxetan-3-yl)phenyl)-6-((S)-1-((S)-3-fluoropyrrolidin-1-yl)ethyl)-4-(trifluoromethyl)isoindolin-1-one. 2-(3-ethoxy-5-(3-((4-methyl-4H-1,2,4-triazol-3-yl)methyl)oxetan-3-yl)phenyl)-6-(1-((S)-3-fluoropyrrolidin-1-yl)-ethyl)-4-(trifluoromethyl)isoindolin-1-one was separated using IG column with CO₂ and methanol as mobile phase to afford 2-(3-ethoxy-5-(3-((4-methyl-4H-1,2,4-triazol-3-yl)methyl)-oxetan-3-yl)phenyl)-6-((R)-1-((S)-3-fluoropyrrolidin-1-yl)ethyl)-4-(trifluoromethyl)isoindolin-1-one (7 mg) and 2-(3-ethoxy-5-(3-((4-methyl-4H-1,2,4-triazol-3-yl)methyl)oxetan-3-yl)phenyl)-6-((S)-1-((S)-3-fluoropyrrolidin-1-yl)ethyl)-4-(trifluoromethyl)isoindolin-1-one (10 mg).

2-(3-ethoxy-5-(3-((4-methyl-4H-1,2,4-triazol-3-yl)methyl)oxetan-3-yl)phenyl)-6-((R)-1-((S)-3-fluoropyrrolidin-1-yl)ethyl)-4-(trifluoromethyl)isoindolin-1-one. ¹H NMR (500 MHz, DMSO-d₆) δ 8.13 (s, 1H), 7.90 (d, J=24.4 Hz, 2H), 7.47 (t, J=2.1 Hz, 1H), 6.84 (t, J=1.6 Hz, 1H), 6.22 (t, J=1.8 Hz, 2H), 5.22-5.06 (m, 1H), 5.00 (s, 2H), 4.91-4.76 (m, 4H), 3.88 (q, J=7.0 Hz, 3H), 3.56 (q, J=6.5 Hz, 2H), 3.43 (s, 2H), 2.58-2.50 (m, 2H), 2.36-2.30 (m, 2H), 2.10-1.99 (m, 1H), 1.87-1.76 (m, 1H), 1.29 (d, J=6.5 Hz, 3H), 1.24 (t, J=6.9 Hz, 3H); LCMS: C₃₀H₃₃F₄N₅O₃ requires: 587, found: m/z=588 [M+H]⁺.

2-(3-ethoxy-5-(3-((4-methyl-4H-1,2,4-triazol-3-yl)methyl)oxetan-3-yl)phenyl)-6-((S)-1-((S)-3-fluoropyrrolidin-1-yl)ethyl)-4-(trifluoromethyl)isoindolin-1-one. ¹H NMR (500 MHz, DMSO-d₆) δ 8.13 (s, 1H), 7.90 (d, J=24.4 Hz, 2H), 7.47 (t, J=2.1 Hz, 1H), 6.84 (t, J=1.6 Hz, 1H), 6.22 (t, J=1.8 Hz, 2H), 5.22-5.07 (m, 1H), 5.00 (s, 2H), 4.92-4.78 (m, 4H), 3.88 (q, J=7.0 Hz, 3H), 3.56 (q, J=6.5 Hz, 2H), 3.43 (s, 2H), 2.57-2.50 (m, 2H), 2.37-2.32 (m, 2H), 2.10-1.99 (m, 1H), 1.86-1.74 (m, 1H), 1.29 (d, J=6.5 Hz, 3H), 1.24 (t, J=6.9 Hz, 3H); LCMS: C₃₀H₃₃F₄N₅O₃ requires: 587, found: m/z=588 [M+H]⁺.

### Example 692: 4-cyclopropyl-6-methyl-2-(3-(3-((4-methyl-4H-1,2,4-triazol-3-yl)methyl)oxetan-3-yl)phenyl)-2,3-dihydro-1H-pyrrolo[3,4-c]pyridin-1-one

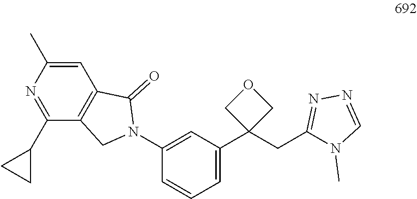 [SMILES: Cc1cc2c(c(C3CC3)n1)CN(c1cccc(C3(Cc4nncn4C)COC3)c1)C2=O]

Step 1: Synthesis of ethyl 3-cyano-2-cyclopropyl-6-methylpyridine-4-carboxylate. A solution of ethyl 2-chloro-3-cyano-6-methylpyridine-4-carboxylate (5.00 g, 22.26 mmol) and tetrakis(triphenylphosphine) palladium (2.57 g, 2.23 mmol) in THF (150 mL) was added bromo(cyclopropyl)zinc (33 mL of 0.5 M solution in THF, 66.7 mmol). The solution was heated at 60-65° C. for 19 h. The solution was allowed to cool to rt, then poured into saturated aqueous ammonium chloride and extracted with EtOAc (3×). The combined organic phases were dried, filtered and concentrated onto Celite. The crude material was purified by chromatography to give the title compound (4.60 g, 90%).

Step 2: Synthesis of 4-cyclopropyl-6-methyl-2H,3H-pyrrolo[3,4-c]pyridin-1-one. To a solution of ethyl 3-cyano-2-cyclopropyl-6-methylpyridine-4-carboxylate (1.00 g, 4.34 mmol) in ethanol (50 mL) under nitrogen was added raney nickel (slurry, 0.2 mL). The reaction was put under a hydrogen balloon and stirred for 3 hours. The suspension is then filtered over celite and concentrated. The crude material was purified by chromatography to give the title compound (620 mg, 76%).

Step 3: Synthesis of 4-cyclopropyl-6-methyl-2-(3-(3-((4-methyl-4H-1,2,4-triazol-3-yl)methyl)oxetan-3-yl)phenyl)-2,3-dihydro-1H-pyrrolo[3,4-c]pyridin-1-one. The coupling reaction was carried out in a similar fashion to Example 168, step 1 using 3-{[3-(3-bromophenyl)oxetan-3-yl]methyl}-4-methyl-4H-1,2,4-triazole (90 mg, 0.29 mmol) and 4-cyclopropyl-6-methyl-1H,2H,3H-pyrrolo[3,4-c]pyridin-1-one (60 mg, 0.32 mmol) as reactants to afford the title compound (39 mg, 32%) as an off-white solid. ¹H NMR (500 MHz, DMSO-d₆) δ 8.13 (s, 1H), 7.87 (ddd, J=8.3, 2.2, 1.0 Hz, 1H), 7.36 (t, J=1.9 Hz, 1H), 7.34-7.23 (m, 2H), 6.70 (dt, J=7.9, 1.1 Hz, 1H), 5.00 (s, 2H), 4.86 (dd, J=32.9, 6.1 Hz, 4H), 3.44 (s, 2H), 3.25 (s, 3H), 2.85 (s, 3H), 2.11 (tt, J=7.9, 4.8 Hz, 1H), 1.08-0.94 (m, 4H); LCMS: C₂₄H₂₅N₅O₂ requires: 415, found: m/z=416 [M+H]⁺.

### Example 693: 6-((3,3-difluoropiperidin-1-yl)methyl)-2-(3-(3-((4-methyl-4H-1,2,4-triazol-3-yl)methyl)oxetan-3-yl)phenyl)-4-(trifluoromethyl)isoindolin-1-one

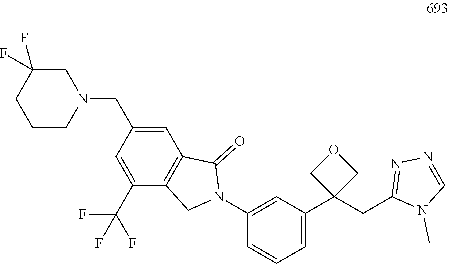 [SMILES: Cn1cnnc1CC1(c2cccc(N3Cc4c(cc(CN5CCCC(F)(F)C5)cc4C(F)(F)F)C3=O)c2)COC1]

Step 1: Synthesis of 6-(hydroxymethyl)-4-(trifluoromethyl)-2,3-dihydroisoindol-1-one. To a solution of methyl 2-(bromomethyl)-5-(hydroxymethyl)-3-(trifluoromethyl)benzoate (10.00 g, 30.57 mmol) in methanol (100 mL) was added ammonia (50 mL, 7N in Methanol). The reaction mixture was stirred at rt for 12 h and evaporated to dryness. The solution was partitioned between EtOAc and water, and then extracted with EtOAc twice. The layers were dried, filtered, and concentrated. The solid was purified by chromatography to yield the title compound (5.50 g, 78%) as a white solid.

Step 2: Synthesis of 6-(chloromethyl)-4-(trifluoromethyl)-2,3-dihydroisoindol-1-one. 6-(hydroxymethyl)-4-(trifluoromethyl)-2,3-dihydroisoindol-1-one (5.50 g, 23.79 mmol) and thionyl chloride (38 g, 316.75 mmol) were added together and stirred for 2 hours at 70° C. The solution was cooled to rt and concentrated. DCM was added and the suspension was concentrated twice to give the title compound.

Step 3: Synthesis of 6-[(3,3-difluoropiperidin-1-yl)methyl]-4-(trifluoromethyl)-2,3-dihydroisoindol-1-one. To a stirring solution of 6-(chloromethyl)-4-(trifluoromethyl)-2,3-dihydroisoindol-1-one (100 mg, 0.40 mmol) and 3,3-difluoropiperidine hydrochloride (126 mg, 0.80 mmol) in acetonitrile (4 mL) was added potassium carbonate (220 mg, 1.60 mmol) and potassium iodide (70 mg, 0.40 mmol). The suspension was stirred at 80° C. for 12h. The solution was partitioned between DCM and water, and then extracted with DCM twice. The layers were dried, filtered, and concentrated. The residue was purified by chromatography to give the title compound (70 mg, 52%).

Step 4: Synthesis of 6-((3,3-difluoropiperidin-1-yl)methyl)-2-(3-(3-((4-methyl-4H, 2,4-triazol-3-yl)methyl)oxetan-3-yl)phenyl)-4-(trifluoromethyl)isoindolin-1-one. The coupling reaction was carried out in a similar fashion to Example 168, step 1 using 3-{[3-(3-bromophenyl)oxetan-3-yl]methyl}-4-methyl-1,2,4-triazole (28 mg, 0.09 mmol) and 6-[(3,3-difluoropiperidin-1-yl)methyl]-4-(trifluoromethyl)-2,3-dihydroisoindol-1-one (30 mg, 0.09 mmol) as reactants to afford the title compound (13 mg, 26%) as an off-white solid. ¹H NMR (500 MHz, DMSO-d6) δ 8.11 (s, 1H), 7.96-7.91 (m, 1H), 7.88 (s, 1H), 7.81 (ddd, J=8.3, 2.3, 0.9 Hz, 1H), 7.33 (t, J=2.0 Hz, 1H), 7.28 (t, J=7.9 Hz, 1H), 6.69 (dt, J=7.5, 1.2 Hz, 1H), 5.03 (s, 2H), 4.89 (d, J=6.1 Hz, 2H), 4.82 (d, J=6.0 Hz, 2H), 3.74 (s, 2H), 3.44 (s, 2H), 2.83 (s, 3H), 2.61 (t, J=11.5 Hz, 2H), 2.40-2.37 (m, 2H), 1.82 (td, J=14.0, 7.0 Hz, 2H), 1.61 (d, J=6.7 Hz, 2H); LCMS: C₂₈H₂₈F₅N₅O₂ requires: 561, found: m/z=562 [M+H]⁺.

### Example 694: 2-cyclopropyl-6-methyl-N-(3-(1-((4-methyl-4H-1,2,4-triazol-3-yl)methyl)cyclobutyl)phenyl)pyrimidine-4-carboxamide

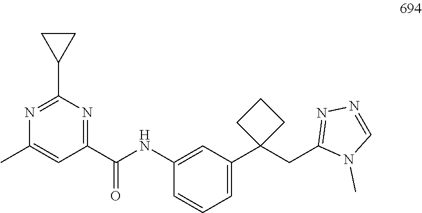 [SMILES: Cc1cc(C(=O)Nc2cccc(C3(Cc4nncn4C)CCC3)c2)nc(C2CC2)n1]

Step 1: Synthesis of ethyl 2-[1-(3-bromophenyl)cyclobutyl]acetate: To a stirred mixture of bis(chlororhodiumcarbaldehyde) (0.35 g, 1.04 mmol) in dioxane (10 mL) was added KOH (7 mL, 2M in water) dropwise at 0˜10° C. The mixture was stirred at 0˜10° C. for 20 min. Then to the mixture was added (3-bromophenyl)boronic acid (5.73 g, 28.53 mmol) and ethyl 2-cyclobutylideneacetate (2.0 g, 14.27 mmol) at 0˜10° C. The mixture was stirred at 25° C. for 16 h under nitrogen. The mixture was quenched by the addition of water and extracted with EtOAc. The combined organic layer was washed with brine, dried, filtered and concentrated under vacuum. The residue was purified by chromatography to afford the title compound (1.4 g, 33%) as a light-yellow oil.

Step 2: Synthesis of 2-[1-(3-bromophenyl)cyclobutyl]acetohydrazide: To a solution of ethyl 2-[1-(3-bromophenyl)cyclobutyl]acetate (3.0 g, 10.1 mmol) in ethanol (35 mL) was added hydrazine hydrate (10.1 mL, 80%). The solution was stirred at 25° C. for 12 h. The solvents were removed under reduced pressure to afford the title compound and then used in the next step without purification.

Step 3: Synthesis of 2-(2-(1-(3-bromophenyl)cyclobutyl)acetyl)-N-methylhydrazine-1-carbothioamide: To a stirred mixture of 2-[1-(3-bromophenyl)cyclobutyl]-acetohydrazide (1.50 g, 5.30 mmol) in EtOH (30 ml) was added isothiocyanatomethane (0.39 g, 5.33 mmol) in portions at 0-5° C. The mixture was stirred at 60° C. for 16 h. The mixture was cooled down to rt and extracted with EtOAc (3×). The combined organic layer was washed with brine, dried, filtered and concentrated under vacuum to afford the title compound and then used in the next step without purification.

Step 4: Synthesis 25-[[1-(3-bromophenyl)cyclobutyl]methyl]-4-methyl-4H-1,2,4-triazole-3-thiol: To a stirred mixture of 2-(2-(1-(3-bromophenyl)cyclobutyl)acetyl)-N-methylhydrazine-1-carbothioamide (1.80 g, 5.05 mmol) in EtOH (25 mL) was added sodium hydroxide (25.00 mL, 2M in water) dropwise at 0° C. The mixture was stirred at 25° C. for 2 h. The mixture was acidified to pH 1 with concentrated hydrochloric acid. The solids were collected by filtration and washed with water and then dried under reduced pressure to afford the title compound and then used in the next step without purification.

Step 5: 3-[[1-(3-bromophenyl)cyclobutyl]methyl]-4-methyl-4H-1,2,4-triazole: To a mixture of 5-[[1-(3-bromophenyl)cyclobutyl]methyl]-4-methyl-4H-1,2,4-triazole-3-thiol (1.35 g, 3.99 mmol) and NaNO₂ (1.65 g, 0.024 mol) was added HNO₃ (24.00 mL, 1 M) at 0° C. The mixture was stirred at rt for 2.5 h. The reaction was quenched by saturated sodium bicarbonate aqueous solution and extracted with EtOAc (3×). The combined organic layer was washed with brine, dried, filtered and concentrated under vacuum. The residue was purified by chromatography to afford the title compound (554 mg, 45%) as a white solid. ¹H NMR (300 MHz, CDCl₃) δ 7.88 (s, 1H), 7.34-7.28 (m, 1H), 7.11-7.03 (m, 2H), 6.75-6.71 (m, 1H), 3.27 (s, 2H), 2.68 (s, 3H), 2.62-2.54 (m, 2H), 2.45-2.24 (m, 3H), 2.01-1.82 (m, 1H). MS(ESI): C₁₄H₆BrN₃ requires: 305, found: m/z=306 [M+H]⁺.

Step 6: Synthesis of 2-cyclopropyl-6-methyl-N-(3-(1-((4-methyl-4H-1,2,4-triazol-3-yl)methyl)cyclobutyl)phenyl)pyrimidine-4-carboxamide. The coupling reaction was carried out in a similar fashion to Example 168, step 1 using 3-{[1-(3-bromophenyl)cyclobutyl]methyl}-4-methyl-1,2,4-triazole (75 mg, 0.24 mmol) and 2-cyclopropyl-6-methylpyrimidine-4-carboxamide (65 mg, 0.37 mmol) as reactants to afford the title compound (32 mg, 33%) as a white solid. ¹H NMR (500 MHz, Acetonitrile-d₃) δ 9.88 (s, 1H), 7.86 (s, 1H), 7.77 (s, 1H), 7.71 (dd, J=7.8, 1.3 Hz, 1H), 7.34-7.22 (m, 2H), 6.70 (dt, J=7.8, 1.3 Hz, 1H), 3.22 (s, 2H), 2.71 (s, 3H), 2.64-2.57 (m, 2H), 2.55 (s, 3H), 2.43 (dt, J=12.1, 9.0 Hz, 2H), 2.34 (ddd, J=12.9, 8.3, 4.8 Hz, 2H), 2.29-2.22 (m, 1H), 1.22 (tt, J=5.8, 2.9 Hz, 2H), 1.18-1.12 (m, 2H); LCMS: C₂₃H₂₆N₆O requires: 402, found: m/z=403 [M+H]⁺.

### Example 695: 6-((5-azaspiro[2.4]heptan-5-yl)methyl)-2-(3-(1-((4-methyl-4H-1,2,4-triazol-3-yl)methyl)cyclobutyl)phenyl)-4-(trifluoromethyl)isoindolin-1-one

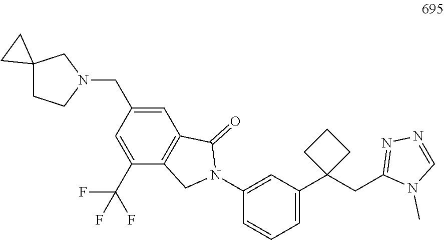 [SMILES: Cn1cnnc1CC1(c2cccc(N3Cc4c(cc(CN5CCC6(CC6)C5)cc4C(F)(F)F)C3=O)c2)CCC1]

Step 1: Synthesis of 6-((5-azaspiro[2.4]heptan-5-yl)methyl)-4-(trifluoromethyl)isoindolin-1-one. To a stirring solution of 6-(chloromethyl)-4-(trifluoro-methyl)-2,3-dihydroisoindol-1-one (499 mg, 2.00 mmol) and 5-azaspiro[2.4]heptane hydrochloride (293 mg, 2.20 mmol) in acetonitrile (20 mL) was added potassium carbonate (1.12 g, 8.00 mmol) and potassium iodide (332 mg, 2.00 mmol). The suspension was stirred at 80° C. for 12h. The solution was partitioned between DCM and water, and then extracted with DCM twice. The layers were dried, filtered, and concentrated. The crude material was purified by silica gel column chromatography using methanol in DCM to give the title compound (550 mg, 89%).

Step 2: 6-((5-azaspiro[2.4]heptan-5-yl)methyl)-2-(3-(1-((4-methyl-4H-1,2,4-triazol-3-yl)methyl)cyclobutyl)phenyl)-4-(trifluoromethyl)isoindolin-1-one. The coupling reaction was carried out in a similar fashion to Example 168, step 1 using 3-{[1-(3-bromophenyl)cyclobutyl]-methyl}-4-methyl-1,2,4-triazole (72 mg, 0.24 mmol) and 6-{5-azaspiro[2.4]heptan-5-ylmethyl}-4-(trifluoromethyl)-2,3-dihydroisoindol-1-one (114 mg, 0.37 mmol) as reactants to afford the title compound (20 mg, 26%) as an off-white solid. ¹H NMR (500 MHz, Acetonitrile-d₃) δ 7.99 (s, 1H), 7.93 (s, 1H), 7.86 (s, 1H), 7.81 (dt, J=8.1, 1.5 Hz, 1H), 7.32 (t, J=7.9 Hz, 1H), 7.28 (t, J=2.0 Hz, 1H), 6.77 (dt, J=7.8, 1.2 Hz, 1H), 4.95 (d, J=1.5 Hz, 2H), 3.81 (s, 2H), 3.24 (s, 2H), 2.76 (t, J=6.9 Hz, 2H), 2.70 (s, 3H), 2.63 (ddt, J=12.4, 8.9, 3.1 Hz, 2H), 2.54 (s, 2H), 2.45 (qd, J=9.1, 2.5 Hz, 2H), 2.33-2.23 (m, 2H), 1.83 (t, J=6.9 Hz, 2H), 0.60-0.49 (m, 4H); LCMS: C₃₀H₃₂F₃N₅O requires: 535, found: m/z=536 [M+H]⁺.

### Example 696: 2-(3-(3-((4-methyl-4H-1,2,4-triazol-3-yl)methyl)oxetan-3-yl)phenyl)-4-(trifluoromethyl)-2,3-dihydro-1H-pyrrolo[3,4-c]pyridin-1-one

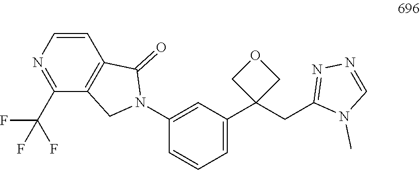 [SMILES: Cn1cnnc1CC1(c2cccc(N3Cc4c(ccnc4C(F)(F)F)C3=O)c2)COC1]

The indolone formation reaction was carried out in a manner similar to 260, step 2 using 3-{3-[(4-methyl-4H-1,2,4-triazol-3-yl)methyl]oxetan-3-yl}aniline (75 mg, 0.31 mmol) and methyl 3-(bromomethyl)-2-(trifluoromethyl)pyridine-4-carboxylate (100 mg, 0.34 mmol) as reactants to afford the title compound (5 mg, 4%) as an off-white solid. ¹H NMR (500 MHz, DMSO-d₆) δ 8.91 (d, J=4.8 Hz, 1H), 8.12 (s, 1H), 8.03 (d, J=4.8 Hz, 1H), 7.81 (ddd, J=8.2, 2.3, 1.0 Hz, 1H), 7.38 (t, J=1.9 Hz, 1H), 7.31 (t, J=7.9 Hz, 1H), 6.75 (dt, J=7.9, 1.1 Hz, 1H), 5.18 (s, 2H), 4.86 (dd, J=39.7, 6.1 Hz, 4H), 3.45 (s, 2H), 2.86 (s, 3H); LCMS: C₂₁H₁₈F₃N₅O₂ requires: 429, found: m/z=430 [M+H]⁺.

### Example 697: 6-(hydroxy(phenyl)methyl)-2-(3-(3-((4-methyl-4H-1,2,4-triazol-3-yl)methyl)oxetan-3-yl)phenyl)-4-(trifluoromethyl)isoindolin-1-one

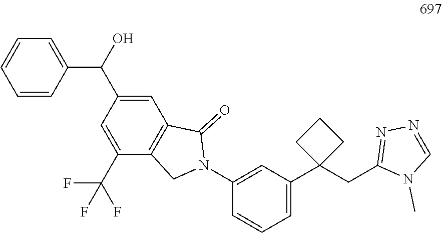 [SMILES: Cn1cnnc1CC1(c2cccc(N3Cc4c(cc(C(O)c5ccccc5)cc4C(F)(F)F)C3=O)c2)CCC1]

To a stirring solution of 2-(3-{3-[(4-methyl-4H-1,2,4-triazol-3-yl)methyl]oxetan-3-yl}phenyl)-3-oxo-7-(trifluoromethyl)-2,3-dihydro-1H-isoindole-5-carbaldehyde (40 mg, 0.09 mmol) in THF (1 mL) was added bromo(phenyl)magnesium (0.10 mL, 0.10 mmol, 1M in THF). The reaction was stirred for 1 h and then quench with saturated ammonium chloride. The residue was purified by HPLC (acetonitrile in water with 0.1% trifluoroacetic acid) to give the title compound (6 mg, 12%). ¹H NMR (500 MHz, DMSO-d₆) δ 8.10 (s, 1H), 7.95 (d, J=9.5 Hz, 2H), 7.79 (ddd, J=8.1, 2.3, 1.0 Hz, 1H), 7.40-7.34 (m, 2H), 7.32-7.23 (m, 3H), 7.22-7.14 (m, 1H), 6.69 (dt, J=7.7, 1.2 Hz, 1H), 6.46 (s, 1H), 6.24 (d, J=4.2 Hz, 1H), 5.92 (d, J=4.1 Hz, 1H), 5.00 (s, 2H), 4.88 (d, J=6.0 Hz, 2H), 4.80 (d, J=6.0 Hz, 2H), 3.43 (s, 2H), 2.82 (s, 3H); LCMS: C₂₉H₂₅F₃N₄O₃ requires: 534, found: m/z=535 [M+H]⁺.

### Example 698: 2-cyclopropyl-N-(3-cyclopropyl-5-(3-((4-methyl-4H-1,2,4-triazol-3-yl)methyl)oxetan-3-yl)phenyl)-6-methylpyrimidine-4-carboxamide

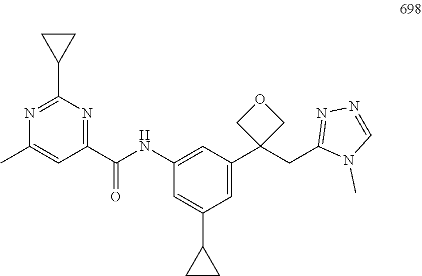 [SMILES: Cc1cc(C(=O)Nc2cc(C3CC3)cc(C3(Cc4nncn4C)COC3)c2)nc(C2CC2)n1]

Step 1: Synthesis of ethyl 2-(3-(3,5-dibromophenyl)oxetan-3-yl)acetate. Aqueous KOH (120 mL, 1.5 M, 0.18 mol) was added to a suspension of [Rh(COD)CI]₂ (3.0 g, 6.1 mmol) in dioxane (100 ml) and the mixture was stirred for 30 min. (3,5-dibromophenyl)boronic acid (50 g, 178.8 mmol) and successively ethyl 2-(oxetan-3-ylidene)acetate (17 g, 120 mmol) in dioxane (40 mL) were added and the reaction mixture was stirred at rt for 16 h under nitrogen. The reaction was quenched by the addition of HCl (1 N) to pH=67 and then extracted with EtOAc (3×). The combined organic layer was washed with brine, dried, and concentrated. The residue was purified by chromatography with EtOAc in petroleum ether to afford ethyl 2-(3-(3,5-dibromophenyl)oxetan-3-yl)acetate (25.9 g, 57%).

Step 2: Synthesis of 2-(3-(3,5-dibromophenyl)oxetan-3-yl)acetohydrazide. A mixture of ethyl 2-(3-(3,5-dibromophenyl)oxetan-3-yl)acetate (25.9 g, 68.5 mmol) in ethanol (130 mL) and hydrazine hydrate (130 mL) was stirred at 80° C. for 16 h. The solvent was removed under vacuum. The residue was triturated with EtOAc/petroleum ether (1/10) to afford the title compound, which was used in the next step without purification.

Step 3: Synthesis of 2-(2-(3-(3,5-dibromophenyl)oxetan-3-yl)acetyl)-N-methylhydrazine-1-carbothioamide. To a solution of 2-(3-(3,5-dibromophenyl)oxetan-3-yl)acetohydrazide (27.0 g, crude) in THF (100 mL) was added isothiocyanatomethane (3.8 g, 52.5 mmol). The solution was stirred at rt for 16 h. The solvent was removed under vacuum to afford 2-(2-(3-(3,5-dibromophenyl)oxetan-3-yl)acetyl)-N-methylhydrazine-1-carbothioamide which was used in the next step without purification.

Step 4: Synthesis of 5-((3-(3,5-dibromophenyl)oxetan-3-yl)methyl)-4-methyl-4H-1,2,4-triazole-3-thiol. To a mixture of 2-(2-(3-(3,5-dibromophenyl)oxetan-3-yl)acetyl)-N-methylhydrazine-1-carbothioamide (32.0 g, crude) in THF (200 mL) was added aqueous sodium hydroxiede (1M, 200 mL). The mixture was stirred at rt for 2 h. The mixture was diluted with water and acidified to pH 5 by HCl (1 N), then extracted with EtOAc. The combined organic layer was washed with brine, dried, concentrated under vacuum to afford the title compound which was used in the next step without purification.

Step 5: Synthesis of 3-((3-(3,5-dibromophenyl)oxetan-3-yl)methyl)-4-methyl-4H-1,2,4-triazole. To a mixture of 5-((3-(3,5-dibromophenyl)oxetan-3-yl)methyl)-4-methyl-4H-1,2,4-triazole-3-thiol (21.8 g, crude) in DCM (220 mL) and acetic acid (44 mL) was added H₂O₂ (5.31 g, 156 mmol, 30%) dropwise at 0° C. The reaction mixture was stirred at rt for 2 h. The mixture was adjusted to pH 7 with saturated NaHCO₃ aqueous solution and then the mixture was extracted with EtOAc. The combined organic layer was washed with brine, dried and concentrated under vacuum. The residue was purified by silica gel flash chromatography to afford the title compound (7.9 g, 30% over 4 steps).

Step 6: N-(3-bromo-5-{3-[(4-methyl-1,2,4-triazol-3-yl)methyl]oxetan-3-yl}phenyl)-2-cyclopropyl-6-methylpyrimidine-4-carboxamide. To a solution of 3-{[3-(3,5-dibromophenyl)oxetan-3-yl]methyl}-4-methyl-4H-1,2,4-triazole (100 mg, 0.26 mmol), [5-(diphenylphosphanyl)-9,9-dimethyl-9H-xanthen-4-yl]diphenylphosphane (30 mg, 0.05 mmol), and 2-cyclopropyl-6-methylpyrimidine-4-carboxamide (45 mg, 0.26 mmol) in 1,4-dioxane (3 mL) were added cesium carbonate (168 mg, 0.52 mmol) and (acetyloxy)palladio acetate (5.80 mg, 0.03 mmol). The mixture was stirred at 120° C. for 1 h under nitrogen. The suspension was filtered through celite and purified by chromatography to give the title compound (65 mg, 52%)

Step 6: 2-cyclopropyl-N-(3-cyclopropyl-5-(3-((4-methyl-4H-1,2,4-triazol-3-yl)methyl)oxetan-3-yl)phenyl)-6-methylpyrimidine-4-carboxamide. To a solution of N-(3-bromo-5-{3-[(4-methyl-1,2,4-triazol-3-yl)methyl]oxetan-3-yl}phenyl)-2-cyclopropyl-6-methylpyrimidine-4-carboxamide (70 mg, 0.14 mmol), 2-cyclopropyl-4,4,5,5-tetramethyl-1,3,2-dioxaborolane (73 mg, 0.43 mmol), and [1,1′-Bis(diphenylphosphino)ferrocene]dichloro palladium(II), complex with DCM (12 mg, 0.01 mmol) in 1,4-dioxane (0.2 mL) and water (0.5 mL) was added cesium carbonate (141 mg, 0.43 mmol). The reaction was heated at 100° C. for 12 h. The suspension was filtered through celite and purified by HPLC to afford the title compound (19 mg, 30%). ¹H NMR (500 MHz, DMSO-d₆) δ 10.32 (s, 1H), 8.22 (s, 1H), 7.70 (s, 1H), 7.52 (t, J=1.7 Hz, 1H), 7.28 (t, J=1.8 Hz, 1H), 6.27 (t, J=1.6 Hz, 1H), 4.92 (d, J=5.9 Hz, 2H), 4.85 (d, J=6.0 Hz, 2H), 3.47 (s, 2H), 2.89 (s, 3H), 2.53 (s, 3H), 2.33 (td, J=8.3, 4.1 Hz, 1H), 1.88-1.82 (m, 1H), 1.21-1.06 (m, 4H), 0.97-0.87 (m, 2H), 0.61-0.48 (m, 2H); LCMS: C₂₅H₂₈N₆O₂ requires: 444, found: m/z=445 [M+H]⁺.

### Example 699: (S)-6-((3-fluoropyrrolidin-1-yl)methyl)-N-(3-(3-((4-methyl-4H-1,2,4-triazol-3-yl)methyl)oxetan-3-yl)phenyl)-2-(trifluoromethyl)pyrimidine-4-carboxamide

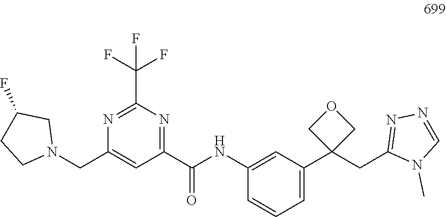 [SMILES: Cn1cnnc1CC1(c2cccc(NC(=O)c3cc(CN4CC[C@H](F)C4)nc(C(F)(F)F)n3)c2)COC1]

Step 1: Synthesis of 4-chloro-6-methyl-2-(trifluoromethyl)pyrimidine. To a solution of 4,6-dichloro-2-(trifluoromethyl)pyrimidine (2.2 g, 10.0 mmol) in toluene (30 mL) and water (3 mL) were added sodium carbonate (3.2 g, 29.7 mmol), potassium methyltrifluoroborate (1.4 g, 11.5 mmol) and Pd(PPh₃)₄ (577 mg, 0.5 mmol). The solution was stirred at 90° C. for 16 h. The mixture was diluted with water and extracted with EtOAc (3×). The combined organic layer was washed with brine, dried and concentrated under vacuum. The residue was purified by chromatography to afford 4-chloro-6-methyl-2-(trifluoromethyl)pyrimidine (600 mg, 31%).

Step 2: Synthesis of methyl 6-methyl-2-(trifluoromethyl)pyrimidine-4-carboxylate. To a mixture of 4-chloro-6-methyl-2-(trifluoromethyl)pyrimidine (2.27 g, 11.5 mmol) in methanol (30 mL) were added Pd(dppf)Cl₂ (845 mg, 1.2 mmol) and TEA (11.6 g, 115.5 mmol). The mixture was stirred at 70° C. for 16 h under CO atmosphere. The solids were filtered out and the filtrate was concentrated under vacuum. The residue was purified by chromatography to afford the title compound (250 mg, 10%).

Step 3: Synthesis of methyl 6-(bromomethyl)-2-(trifluoromethyl)pyrimidine-4-carboxylate. To a mixture of methyl 6-methyl-2-(trifluoromethyl)pyrimidine-4-carboxylate (240 mg, 1.1 mmol) in EtOAc (3 mL) was added Br₂ (174 mg, 1.1 mmol) at 0° C. The mixture was stirred at 80° C. for 45 min. The mixture was concentrated under vacuum. The residue was dissolved in toluene and then concentrated under vacuum to afford the title compound (244 mg, crude) as a grey solid, which was used in the next step without purification.

Step 4: Synthesis of methyl (S)-6-((3-fluoropyrrolidin-1-yl)methyl)-2-(trifluoromethyl)pyrimidine-4-carboxylate. To a mixture of methyl 6-(bromomethyl)-2-(trifluoromethyl)pyrimidine-4-carboxylate (244 mg, 0.8 mmol) in DCM (2 mL) were added (3S)-3-fluoropyrrolidine hydrochloride (102 mg, 0.8 mmol) and TEA (165 mg, 1.6 mmol). The mixture was stirred at rt for 3 h and then concentrated under vacuum. The residue was purified by HPLC (15-98% acetonitrile in water with 0.1% trifluoroacetic acid) to afford the title compound (55.7 mg, 22%).

Step 5: 6-{[(3S)-3-fluoropyrrolidin-1-yl]methyl}-2-(trifluoromethyl)pyrimidine-4-carboxylic acid; chlorolithium. To a stirring solution of methyl 6-{[(3S)-3-fluoropyrrolidin-1-yl]methyl}-2-(trifluoromethyl)pyrimidine-4-carboxylate (57 mg, 0.19 mmol) in THF (1 mL) was added lithium hydroxide (10 mg, 0.23 mmol). The solution was stirred for 2 hours and then quenched with hydrochloric acid (0.1 mL). The solution was concentrated using lyophilization and used crude in the next step.

Step 6: (S)-6-((3-fluoropyrrolidin-1-yl)methyl)-N-(3-(3-((4-methyl-4H-1,2,4-triazol-3-yl)methyl)oxetan-3-yl)phenyl)-2-(trifluoromethyl)pyrimidine-4-carboxamide. The coupling reaction was carried out in a manner similar to 74 using 6-{[(3S)-3-fluoropyrrolidin-1-yl]methyl}-2-(trifluoromethyl)pyrimidine-4-carboxylic acid; chlorolithium (64 mg, 0.19 mmol) and 3-{3-[(4-methyl-4H-1,2,4-triazol-3-yl)methyl]oxetan-3-yl}aniline (56 mg, 0.23 mmol) as reactants to afford the title compound (34 mg, 34%) as an off-white solid. ¹H NMR (500 MHz, DMSO-d₆) δ 10.53 (s, 1H), 8.27 (s, 1H), 8.13 (s, 1H), 7.77-7.67 (m, 1H), 7.41 (t, J=1.9 Hz, 1H), 7.23 (t, J=7.9 Hz, 1H), 6.65 (dt, J=7.7, 1.3 Hz, 1H), 5.29-5.07 (m, 1H), 4.87 (d, J=6.0 Hz, 2H), 4.79 (d, J=6.0 Hz, 2H), 3.94 (s, 2H), 3.42 (s, 2H), 2.86 (s, 3H), 2.79-2.64 (m, 2H), 2.26-2.01 (m, 2H), 2.01-1.74 (m, 2H); LCMS: C₂₄H₂₅N₇O₂ requires: 519, found: m/z=520 [M+H]⁺.

### Example 700: 6-((4-fluoropiperidin-1-yl)methyl)-2-(3-(3-((4-methyl-4H-1,2,4-triazol-3-yl)oxy)oxetan-3-yl)phenyl)-4-(trifluoromethyl)isoindolin-1-one

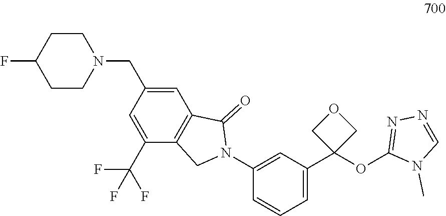 [SMILES: Cn1cnnc1OC1(c2cccc(N3Cc4c(cc(CN5CCC(F)CC5)cc4C(F)(F)F)C3=O)c2)COC1]

Step 1: 3-oxo-7-(trifluoromethyl)-1,2-dihydroisoindole-5-carbaldehyde. To a stirring solution of methyl 2-(bromomethyl)-5-formyl-3-(trifluoromethyl)benzoate (500 mg, 1.54 mmol) was added ammonia (7N in Methanol, 5 mL). The reaction mixture was stirred at rt for 1 h and evaporated to dryness. The solution was partitioned between EtOAc and water, and then extracted with EtOAc twice. The layers were dried, filtered, and concentrated. The residue was purified by chromatography to give the title compound (100 mg, 28%).

Step 2: 6-[(4-fluoropiperidin-1-yl)methyl]-4-(trifluoromethyl)-2,3-dihydroisoindol-1-one. To a stirring solution of 3-oxo-7-(trifluoromethyl)-1,2-dihydroisoindole-5-carbaldehyde (138 mg, 0.60 mmol), 4-fluoropiperidine hydrochloride (252 mg, 1.81 mmol) and triethylamine (253 uL, 1.81 mmol) in DCM (5 mL) was added sodium triacetoxyborohydride (382 mg, 1.81 mmol). The mixture was allowed to stir at rt for about 12 h. The solution was partitioned between DCM and saturated ammonium chloride, and then extracted with DCM twice. The layers were dried, filtered, and concentrated. The residue was purified by chromatography to give the title compound (90 mg, 47%).

Step 3: 3-(3-bromophenyl)oxetan-3-ol. To a stirring solution of 3-oxetanone (4.5 g, 62.5 mmol) in THF (500 mL) at 0° C. was added bromo(3-bromophenyl)magnesium (100 mL, 25.00 mmol. 0.5 M in ether). The reaction was allowed to reach rt over 12 hours. The reaction was quenched with saturated ammonium chloride and extracted with EtOAc (3×). The layers were combined, dried, filtered, and concentrated. The crude was purified using chromatography to afford the title compound (3.20 g, 55.9%) as a colorless oil.

Step 4: 3-{[3-(3-bromophenyl)oxetan-3-yl]oxy}-4-methyl-1,2,4-triazole. To a solution of 3-(3-bromophenyl)oxetan-3-ol (127 mg, 0.55 mmol) in DMF (1 mL) was added sodium hydride (26. mg, 0.67 mmol, 60%). The suspension was stirred for 1 h. After addition of 3-bromo-4-methyl-1,2,4-triazole (269 mg, 1.66 mmol), the reaction was stirred overnight. The reaction was quenched with an aqueous ammonium chloride solution and then extracted with DCM (3×). The combined organic layer was dried, filtered and concentrated under vacuum. The residue was purified by chromatography to afford the title compound (112 mg, 65%)

Step 5: 6-((4-fluoropiperidin-1-yl)methyl)-2-(3-(3-((4-methyl-4H-1,2,4-triazol-3-yl)oxy)oxetan-3-yl)phenyl)-4-(trifluoromethyl)isoindolin-1-one. The coupling reaction was carried out in a similar fashion to Example 168, step 1 using 3-{[3-(3-bromophenyl)oxetan-3-yl]oxy}-4-methyl-1,2,4-triazole (55 mg, 0.18 mmol) and 6-[(4-fluoropiperidin-1-yl)methyl]-4-(trifluoromethyl)-2,3-dihydroisoindol-1-one (67 mg, 0.21 mmol) as reactants to afford the title compound (7 mg, 7%) as an off-white solid. ¹H NMR (500 MHz, Acetonitrile-d₃) δ 8.10 (t, J=2.0 Hz, 1H), 7.90 (s, 1H), 7.84-7.78 (m, 1H), 7.72 (ddd, J=8.1, 2.2, 1.1 Hz, 1H), 7.61 (d, J=0.7 Hz, 1H), 7.37 (t, J=7.9 Hz, 1H), 7.35-7.29 (m, 1H), 5.09-5.03 (m, 2H), 4.99-4.94 (m, 2H), 4.89-4.81 (m, 2H), 4.57-4.51 (m, 1H), 3.58 (s, 2H), 3.52 (dd, J=6.7, 0.6 Hz, 3H), 2.59-2.44 (m, 2H), 2.33-2.24 (m, 2H), 1.78-1.64 (m, 4H); LCMS: C₂₇H₂₇F₄N₅O₃ requires: 545, found: m/z=546 [M+H]⁺.

### Example 701: 6-((5-azaspiro[2.4]heptan-5-yl)methyl)-2-(3-(3-(1-(4-methyl-4H-1,2,4-triazol-3-yl)ethyl)oxetan-3-yl)phenyl)-4-(trifluoromethyl)isoindolin-1-one

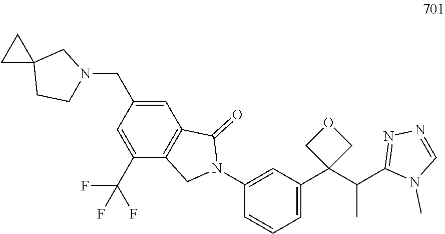 [SMILES: CC(c1nncn1C)C1(c2cccc(N3Cc4c(cc(CN5CCC6(CC6)C5)cc4C(F)(F)F)C3=O)c2)COC1]

Step 1: ethyl 2-[3-(3-nitrophenyl)oxetan-3-yl]propanoate. To a stirring solution of methyl 2-[3-(3-nitrophenyl)oxetan-3-yl]acetate (3.60 g, 13.58 mmol) in THF (100 mF) at −78° C. was added LHMDS (27 mF, 27 mmol, 1M in THF). The solution was stirred for 1 hour. Methyl iodide (1.0 mF, 16.3 mmol) was added at −78° C. and the reaction was allowed to reach rt slowly overnight. The reaction was quenched with ammonium chloride and extracted with EtOAc. The combined organic layer was dried, filtered and concentrated under vacuum. The residue was purified by chromatography to afford the title compound (551 mg, 15%).

Step 2: 2-[3-(3-nitrophenyl)oxetan-3-yl]propanehydrazide. To a stirring solution of ethyl 2-[3-(3-nitrophenyl)oxetan-3-yl]propanoate (600 mg, 2.15 mmol) in ethanol (20 mF) was added hydrazine hydrate (8.43 mF 85.93 mmol). The mixture was stirred at 80° C. for about 24 hours, cooled to rt, and then concentrated. The reaction was diluted with water and extracted with EtOAc (3×). The combined organic layers were washed were dried, filtered, and concentrated under reduced pressure. The crude was used in the next step.

Step 3: 4-methyl-5-{l-[3-(3-nitrophenyl)oxetan-3-yl]ethyl}-1,2,4-triazole-3-thiol. To a solution of 2-[3-(3-nitrophenyl)oxetan-3-yl]propanehydrazide (570 mg, 2.15 mmol) in THF (20 mF) was added methyl isothiocyanate (251 mg, 3.44 mmol). The mixture was stirred at rt for 14 h. KOH (1M, 5 mF) was added and the reaction was stirred overnight. The reaction was diluted with water and extracted with dichlormethane (3×). The combined organic layers were washed were dried, filtered, and concentrated under reduced pressure. The residue was purified by chromatography to give the title compound (250 mg, 36%) as a white solid.

Step 3: 4-methyl-3-{l-[3-(3-nitrophenyl)oxetan-3-yl]ethyl}-1,2,4-triazole. A solution of 4-methyl-5-{l-[3-(3-nitrophenyl)oxetan-3-yl]ethyl}-1,2,4-triazole-3-thiol (250 mg, 0.78 mmol) in DCM (5 mL) and acetic acid (1 mL) was added hydrogen peroxide (0.18 mL, 1.56 mmol, 30%). The reaction mixture was stirred at rt for 16 h and then evaporated to dryness. The reaction was quenched with an aqueous sodium bicarbonate solution and then extracted with DCM (3×). The combined organic layer was dried, filtered and concentrated under vacuum. The residue was purified by chromatography to afford the title compound (120 mg, 53%).

Step 5: 3-{3-[1-(4-methyl-1,2,4-triazol-3-yl)ethyl]oxetan-3-yl}aniline. To a stirring solution of 4-methyl-3-{l-[3-(3-nitrophenyl)oxetan-3-yl]ethyl}-1,2,4-triazole (50 mg, 0.17 mmol) in EtOH (2 mL) and ammonium chloride (0.50 mL) was added iron (96 mg, 1.73 mmol). The reaction mixture was stirred at rt for 16 h. Magnesium sulfate was added to the solution and then the suspension was filtered through celite. The filtrate was concentrated and used without purification.

Step 6: 6-((5-azaspiro[2.4]heptan-5-yl)methyl)-2-(3-(3-(1-(4-methyl-4H-1,2,4-triazol-3-yl)ethyl)oxetan-3-yl)phenyl)-4-(trifluoromethyl)isoindolin-1-one. The reductive amination was carried out in a similar fashion as for 411, step 2 using Example Z (7 mg, 0.01 mmol) and 5-azaspiro[2.4]heptane hydrochloride (4 mg, 0.03 mmol) as reactants to afford the title compound (4 mg, 43%) as a white solid. ¹H NMR (500 MHz, Methanol-d₄) δ 8.11 (s, 1H), 8.06 (s, 1H), 7.99 (s, 1H), 7.74 (dd, J=8.2, 2.1 Hz, 1H), 7.35 (t, J=8.0 Hz, 1H), 7.29 (t, J=2.0 Hz, 1H), 6.66 (d, J=7.9 Hz, 1H), 5.34 (d, J=6.7 Hz, 1H), 5.14 (d, J=6.4 Hz, 1H), 5.09-5.03 (m, 3H), 5.02 (d, J=6.6 Hz, 1H), 3.84 (s, 2H), 3.55 (q, J=7.0 Hz, 1H), 2.94 (s, 3H), 2.81 (t, J=7.0 Hz, 2H), 2.55 (s, 2H), 1.87 (t, J=7.0 Hz, 2H), 1.83 (d, J=7.0 Hz, 3H), 0.56 (s, 4H); LCMS: C₃₀H₃₂F₃N₅O₂ requires: 551, found: m/z=552 [M+H]⁺.

### Example 702: 6-((4-methoxyisoindolin-2-yl)methyl)-2-(3-(3-((4-methyl-4H-1,2,4-triazol-3-yl)methyl)oxetan-3-yl)phenyl)-4-(trifluoromethyl)isoindolin-1-one

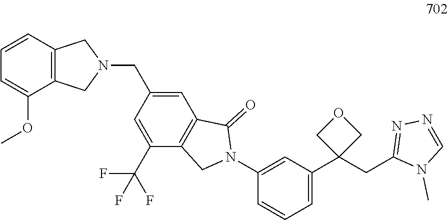 [SMILES: COc1cccc2c1CN(Cc1cc3c(c(C(F)(F)F)c1)CN(c1cccc(C4(Cc5nncn5C)COC4)c1)C3=O)C2]

The reductive amination was carried out in a similar fashion as for 411, step 2 using Example Z (100 mg, 0.22 mmol) and 4-methoxy-2,3-dihydro-1H-isoindole hydrochloride (122 mg, 0.66 mmol) as reactants to afford the title compound (26 mg, 20%) as a white solid. ¹H NMR (500 MHz, Acetonitrile-d₃) δ 8.10 (s, 1H), 8.04 (s, 1H), 7.95-7.90 (m, 2H), 7.38 (dd, J=8.0 Hz, 1H), 7.35 (dd, J=2.0 Hz, 1H), 7.25 (dd, J=1.9 Hz, 1H), 6.85 (dd, J=8.8 Hz, 2H), 6.79 (dt, 0.7=7.9, 1.2 Hz, 1H), 5.05 (d, J=6.1 Hz, 2H), 5.00 (d, J=6.1 Hz, 4H), 4.22-3.85 (m, 6H), 3.82 (s, 3H), 3.55 (s, 2H), 2.89 (s, 3H); LCMS: C₃₂H₃₀F₃N₅O₃ requires: 589, found: m/z=590 [M+H]⁺.

### Example 703: 6-((5-oxa-8-azaspiro[3.5]nonan-8-yl)methyl)-2-(3-(3-((4-methyl-4H-1,2,4-triazol-3-yl)methyl)oxetan-3-yl)phenyl)-4-(trifluoromethyl)isoindolin-1-one

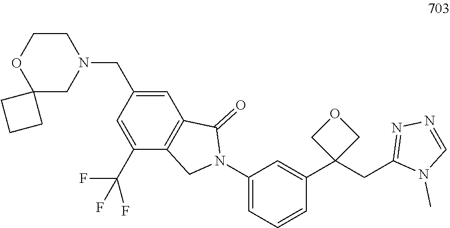 [SMILES: Cn1cnnc1CC1(c2cccc(N3Cc4c(cc(CN5CCOC6(CCC6)C5)cc4C(F)(F)F)C3=O)c2)COC1]

The reductive amination was carried out in a similar fashion as for 411, step 2 using Example Z (100 mg, 0.22 mmol) and 5-oxa-8-azaspiro[3.5]nonane (83 mg, 0.66 mmol) as reactants to afford the title compound (69 mg, 56%) as a white solid. 1H NMR (500 MHz, Acetonitrile-d3) δ 8.03 (s, 1H), 7.97 (s, 1H), 7.92 (d, J=10.1 Hz, 2H), 7.38 (dd, J=8.0 Hz, 1H), 7.35 (dd, J=2.0 Hz, 1H), 6.79 (d, J=7.8, 1.3 Hz, 1H), 5.05 (d, J=6.0 Hz, 2H), 5.00 (d, J=5.9 Hz, 4H), 3.69 (s, 2H), 3.62-3.57 (m, 2H), 3.55 (s, 2H), 2.89 (s, 3H), 2.48-2.35 (m, 4H), 2.08-1.98 (m, 4H), 1.83-1.72 (m, 1H), 1.59-1.47 (m, 1H); LCMS: C₃₀H₃₂F₃N₅O₃ requires: 567, found: m/z=568 [M+H]+.

### Example 704: 2-cyclopropyl-N-(3-(3-((4-(difluoromethyl)-4H-1,2,4-triazol-3-yl)methyl)-oxetan-3-yl)phenyl)-6-methylpyrimidine-4-carboxamide

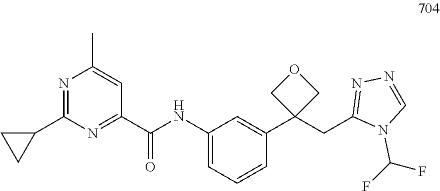 [SMILES: Cc1cc(C(=O)Nc2cccc(C3(Cc4nncn4C(F)F)COC3)c2)nc(C2CC2)n1]

Step 1: [3-(3-bromophenyl)oxetan-3-yl]acetic acid: To a solution of ethyl 2-[3-(3-bromophenyl)oxetan-3-yl]acetate (15.0 g, 50.3 mmol) in tetrahydrofuran (150 mL) and water (120 mL) was added LiOH.H₂O (4.2 g, 100.3 mmol) at 0° C. The mixture was stirred at room temperature for 3 h. The organic solvent was removed under vacuum and then the residue was diluted with water and acidified to pH 3 by HCl (1 N). The mixture was extracted with EtOAc three times. The combined organic layers were washed with brine, dried, and concentrated under vacuum to afford the title compound. The crude product was used for the next step without further purification.

Step 2: 2-[3-(3-bromophenyl)oxetan-3-yl]acetamide: To a mixture of [3-(3-bromo-phenyl)oxetan-3-yl]acetic acid (12.0 g, 44.4 mmol), NH₄Cl (9.5 g, 177.7 mmol), 1-hydroxy-benzotriazole (8.4 g, 62.2 mmol) and EDC HCl (11.0 g, 57.7 mmol) in DMF (140 mL) was added ethyldiisopropylamine (28.7 g, 222 mmol) at 0° C. The resulting mixture was stirred at room temperature for 16 h. Water was added and the solution was extracted with EtOAc. The combined organic layers were washed with brine, dried over Na₂SO₄ and concentrated under vacuum. The residue was purified by flash column chromatography with methanol in DCM to afford the title compound (7.1 g, 59%).

Step 3: (E)-2-(3-(3-bromophenyl)oxetan-3-yl)-N-((dimethylamino)methylene)-acetamide: A solution of 2-[3-(3-bromophenyl)oxetan-3-yl]acetamide (7.1 g, 26.3 mmol) in DMF-DMA (45.0 mL) was stirred at 80° C. for 16 h. The reaction mixture was poured into a mixture of water and brine. The resulting mixture was extracted with EtOAc. The combined organic layers were dried over Na₂SO₄. After filtration and evaporation, the residue was purified by reverse phase C18 flash column chromatography with acetonitrile in water to afford the title compound (5.6 g, 65%).

Step 4: 3-[[3-(3-bromophenyl)oxetan-3-yl]methyl]-4H-1,2,4-triazole: A mixture of (E)-2-(3-(3-bromophenyl)oxetan-3-yl)-N-((dimethylamino)methylene)acetamide (5.6 g, 17.2 mmol) in acetic acid (50 mL) and hydrazine (50 mL, 80%) was stirred at 80° C. for 4 h. The reaction mixture was concentrated and then purified directly by reverse phase C18 flash column chromatography with acetonitrile in water to afford the title compound (1.5 g, 30%).

Step 5: 3-[[3-(3-bromophenyl)oxetan-3-yl]methyl]-4-(difluoromethyl)-1,2,4-triazole: To a solution of 3-[[3-(3-bromophenyl)oxetan-3-yl]methyl]-4H-1,2,4-triazole (1.5 g, 5.1 mmol) in dimethylformamide (10 mL) was added NaH (2.1 g, 52.2 mmol, 60% purity) in portions at 0° C. and stirred for 30 min at room temperature. Then the reaction mixture was stirred at 28° C. for 16 h under CHF₂Cl (gas). The reaction mixture was purified directly by reverse phase flash column chromatography with acetonitrile in water to afford 1.5 g of crude product. The crude product was purified by HPLC with acetonitrile in water to afford the title compound (478 mg, 27%).

Step 6: 2-cyclopropyl-N-(3-(3-((4-(difluoromethyl)-4H-1,2,4-triazol-3-yl)methyl)oxetan-3-yl)phenyl)-6-methylpyrimidine-4-carboxamide. The coupling reaction was carried out in a similar fashion to Example 168, step 1 using 3-{[3-(3-bromophenyl)oxetan-3-yl]methyl}-4-(difluoromethyl)-1,2,4-triazole (100 mg, 0.29 mmol) and 2-cyclopropyl-6-methylpyrimidine-4-carboxamide (77 mg, 0.44 mmol) as reactants afford the title compound (34 mg, 27%) as an off-white solid. ¹H NMR (500 MHz, Acetonitrile-d₃) δ 9.95 (s, 1H), 8.39 (s, 1H), 7.81-7.72 (m, 2H), 7.56-7.51 (m, 1H), 7.34 (dd, J=7.9 Hz, 1H), 6.91-6.87 (m, 1H), 6.89 (t, J=59.1 Hz, 1H), 5.17-4.94 (m, 4H), 3.71 (s, 2H), 2.56 (s, 3H), 2.38-2.31 (m, 1H), 1.25-1.12 (m, 4H); LCMS: C₂₂H₂₂F₂N₆O₂ requires: 440, found: m/z=441 [M+H]⁺.

### Example 705: 2-cyclopropyl-N-(3-{3-[(4-ethyl-1,2,4-triazol-3-yl)methyl]oxetan-3-yl}phenyl)-6-methylpyrimidine-4-carboxamide

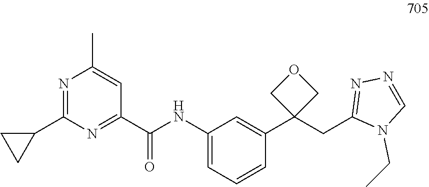 [SMILES: CCn1cnnc1CC1(c2cccc(NC(=O)c3cc(C)nc(C4CC4)n3)c2)COC1]

Step 1: To a stirring solution of 3-{[3-(3-bromophenyl)oxetan-3-yl]methyl}-4H-1,2,4-triazole (400 mg, 1.36 mmol) and potassium carbonate (187 mg, 1.36 mmol) in DMF (4 mL) was added ethyl iodide (0.11 mL 1.36 mmol). The mixture was stirred at 80° C. for about 16 hours. The reaction mixture was diluted with water and extracted with DCM three times. The combined organic layer was washed with water and brine, dried, and concentrated under reduced pressure. The residue was purified by column chromatography B afford a mixture of isomers.

Step 2: 2-cyclopropyl-N-(3-{3-[(4-ethyl-1,2,4-triazol-3-yl)methyl]oxetan-3-yl}phenyl)-6-methylpyrimidine-4-carboxamide. The coupling reaction was carried out in a similar fashion to Example 168, step 1 using 3-{[3-(3-bromophenyl)oxetan-3-yl]methyl}-4-ethyl-1,2,4-triazole (70 mg, 0.22 mmol, mixture of isomers) and 2-cyclopropyl-6-methylpyrimidine-4-carboxamide (39 mg, 0.22 mmol) as reactants afford the title compound (18 mg, 19%) as an off-white solid. ¹H NMR (500 MHz, Acetonitrile-d₃) δ 9.91 (s, 1H), 7.82-7.68 (m, 3H), 7.38 (dd, J=2.0 Hz, 1H), 7.30 (dd, J=7.9 Hz, 1H), 6.74 (dt, J=7.7, 1.3 Hz, 1H), 5.07 (d, J=6.0 Hz, 2H), 4.97 (d, J=6.0 Hz, 2H), 3.57 (d, J=7.9 Hz, 4H), 2.55 (s, 3H), 2.40-2.31 (m, 1H), 1.24-1.13 (m, 4H), 0.99 (t, J=7.2 Hz, 3H); LCMS: C₂₃H₂₆N₆O₂ requires: 418, found: m/z=419 [M+H]⁺.

### Example 706: 2-((2-(3-(3-((4-methyl-4H-1,2,4-triazol-3-yl)methyl)oxetan-3-yl)phenyl)-3-oxo-7-(trifluoromethyl)isoindolin-5-yl)methyl)-1,2,3,4-tetrahydroisoquinoline-6-carbonitrile

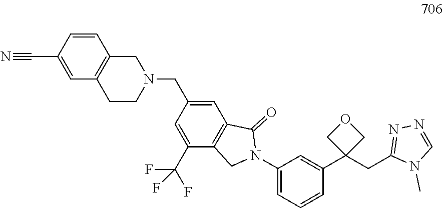 [SMILES: Cn1cnnc1CC1(c2cccc(N3Cc4c(cc(CN5CCc6cc(C#N)ccc6C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)-isoindoline-5-carbaldehyde (100 mg, 0.22 mmol) and 1,2,3,4-tetrahydroisoquinoline-6-carbonitrile (104 mg, 0.66 mmol) as reactants to afford the title compound (50 mg, 37%) as a white solid. ¹H NMR (500 MHz, Acetonitrile-d₃) δ 8.05 (s, 1H), 7.98 (s, 1H), 7.92 (d, J=9.6 Hz, 2H), 7.51 (dd, J=7.9, 1.7 Hz, 1H), 7.42 (d, J=1.6 Hz, 1H), 7.38 (dd, J=8.0 Hz, 1H), 7.36-7.28 (m, 2H), 6.79 (dt, J=7.8, 1.2 Hz, 1H), 5.05 (d, J=6.1 Hz, 2H), 5.00 (d, J=6.0 Hz, 4H), 3.89 (s, 2H), 3.69 (s, 2H), 3.55 (s, 2H), 2.98 (t, J=5.9 Hz, 2H), 2.89 (s, 3H), 2.82 (t, J=5.9 Hz, 2H); LCMS: requires: 598, found: m/z=599 [M+H]⁺.

### Example 707: (S)-2-(3-(3-((4-methyl-4H-1,2,4-triazol-3-yl)methyl)oxetan-3-yl)phenyl)-4-(trifluoromethyl)-6-((2-(trifluoromethyl)morpholino)methyl)isoindolin-1-one

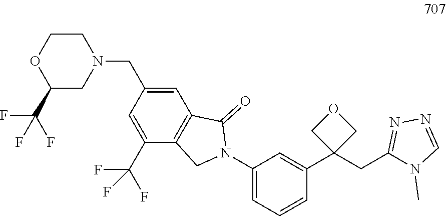 [SMILES: Cn1cnnc1CC1(c2cccc(N3Cc4c(cc(CN5CCO[C@H](C(F)(F)F)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)-isoindoline-5-carbaldehyde (100 mg, 0.22 mmol) and (2S)-2-(trifluoromethyl)morpholine (102 mg, 0.66 mmol) as reactants to afford the title compound (29 mg, 22%) as a white solid, 1H NMR (500 MHz, Acetonitrile-d₃) δ 8.03 (s, 1H), 7.96-7.86 (m, 3H), 7.38 (dd, J=8.0 Hz, 1H), 7.34 (dd, J=2.0 Hz, 1H), 6.79 (d, J=7.8, 1.2 Hz, 1H), 5.05 (d, J=6.1 Hz, 2H), 5.02-4.96 (m, 4H), 4.11 (dtd, J=13.1, 6.6, 2.6 Hz, 1H), 4.04-3.92 (m, 1H), 3.77 (s, 2H), 3.72 (dd, J=11.4, 2.6 Hz, 1H), 3.55 (s, 2H), 2.92 (s, 1H), 2.89 (s, 3H), 2.77-2.69 (m, 1H), 2.36-2.30 (m, 1H), 2.30-2.24 (m, 1H); LCMS: C₂₈H₂₇F₆N₅O₃ requires: 595, found: m/z=596 [M+H]⁺.

### Example 708: rel-6-[(3aR,7aS)-hexahydro-2H-furo[3,2-c]pyridin-5-ylmethyl]-2-(3-{3-[(4-methyl-1,2,4-triazol-3-yl)methyl]oxetan-3-yl}phenyl)-4-(trifluoromethyl)-3H-isoindol-1-one

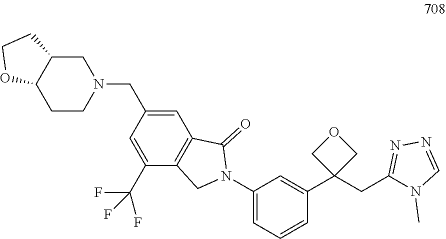 [SMILES: Cn1cnnc1CC1(c2cccc(N3Cc4c(cc(CN5CC[C@@H]6OCC[C@@H]6C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)-isoindoline-5-carbaldehyde (100 mg, 0.22 mmol) and rel-(3aR,7aS)-octahydrofuro[3,2-c]-pyridine hydrochloride (108 mg, 0.66 mmol) as reactants to afford the title compound (50 mg, 40%) as a white solid. ¹H NMR (500 MHz, Acetonitrile-d₃) δ 8.02 (s, 1H), 7.98-7.88 (m, 3H), 7.38 (dd, J=8.0 Hz, 1H), 7.34 (dd, J=2.0 Hz, 1H), 6.79 (dd, J=7.7, 1.4 Hz, 1H), 5.05 (d, J=6.0 Hz, 2H), 4.99 (d, J=6.1 Hz, 4H), 4.00-3.85 (m, 2H), 3.84-3.61 (m, 4H), 3.55 (s, 2H), 2.89 (s, 3H), 2.58 (d, J=51.9 Hz, 1H), 2.40-2.19 (s, 2H), 1.94-1.83 (s, 4H), 1.70-1.56 (m, 1H); LCMS: C₃₀H₃₂F₃N₅O₃ requires: 567, found: m/z=568 [M+H]⁺.

### Example 709: 6-((7,8-dihydro-1,6-naphthyridin-6(5H)-yl)methyl)-2-(3-(3-((4-methyl-4H-1,2,4-triazol-3-yl)methyl)oxetan-3-yl)phenyl)-4-(trifluoromethyl)isoindolin-1-one

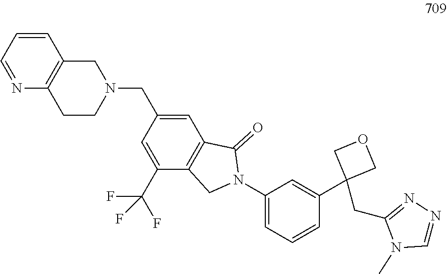 [SMILES: Cn1cnnc1CC1(c2cccc(N3Cc4c(cc(CN5CCc6ncccc6C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)isoindoline-5-carbaldehyde (100 mg, 0.22 mmol) and 5,6,7,8-tetrahydro-1,6-naphthyridine (29 mg, 0.22 mmol) as reactants to afford the title compound (54 mg, 41%) as a white solid. ¹H NMR (500 MHz, Acetonitrile-d₃) δ 8.40 (dd, J=4.9, 1.6 Hz, 1H), 8.08 (s, 1H), 8.02 (s, 1H), 7.92 (d, J=5.9 Hz, 2H), 7.42 (dd, J=7.8, 1.6 Hz, 1H), 7.38 (dd, J=8.0 Hz, 1H), 7.36-7.32 (m, 1H), 7.15 (dd, J=7.7, 4.8 Hz, 1H), 6.79 (d, J=7.6, 1.2 Hz, 1H), 5.05 (d, J=6.0 Hz, 2H), 5.03-4.96 (m, 4), 3.96 (s, 2H), 3.74 (s, 2H), 3.55 (s, 2H), 3.02 (t, J=6.0 Hz, 2H), 2.96 (t, J=5.9 Hz, 2H), 2.89 (s, 3H); LCMS: C₃₁H₂₉F₃N₆O₂ requires: 574, found: m/z=575 [M+H]⁺.

### Example 710: (S)-6-((2-(hydroxymethyl)morpholino)methyl)-2-(3-(3-((4-methyl-4H-1,2,4-triazol-3-yl)methyl)oxetan-3-yl)phenyl)-4-(trifluoromethyl)isoindolin-1-one

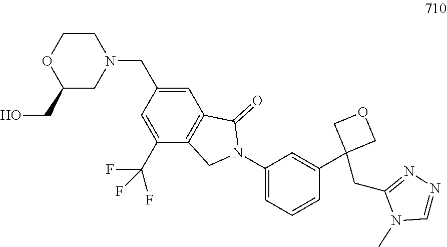 [SMILES: Cn1cnnc1CC1(c2cccc(N3Cc4c(cc(CN5CCO[C@H](CO)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)isoindoline-5-carbaldehyde (100 mg, 0.22 mmol) and (2S)-morpholin-2-ylmethanol (77 mg, 0.66 mmol) as reactants to afford the title compound (53 mg, 43%) as a white solid. ¹H NMR (500 MHz, Acetonitrile-d₃) δ 8.01 (s, 1H), 7.97-7.87 (m, 3H), 7.38 (d, J=7.9 Hz, 1H), 7.36-7.32 (m, 1H), 6.79 (d, J=7.8, 1.3 Hz, 1H), 5.05 (d, J=6.1 Hz, 2H), 5.04-4.96 (m, 4H), 3.86 (ddd, J=11.3, 3.5, 1.8 Hz, 1H), 3.76-3.67 (m, 2H), 3.67-3.60 (m, 1H), 3.55 (s, 2H), 3.52-3.40 (m, 3H), 2.89 (s, 3H), 2.77 (dt, J=11.2, 2.0 Hz, 1H), 2.73-2.64 (m, 2H), 2.22 (td, J=11.3, 3.4 Hz, 1H); LCMS: C₂₈H₃₀F₃N₅O₄ requires: 557, found: m/z=558 [M+H]⁺.

### Example 711: 2-((2-(3-(3-((4-methyl-4H-1,2,4-triazol-3-yl)methyl)oxetan-3-yl)phenyl)-3-oxo-7-(trifluoromethyl)isoindolin-5-yl)methyl)hexahydropyrrolo[1,2-a]pyrazin-6(2H)-one

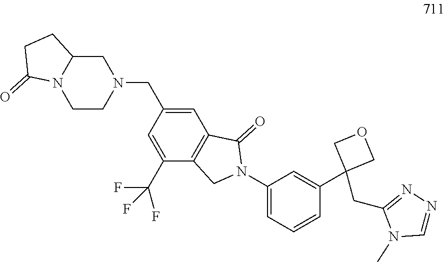 [SMILES: Cn1cnnc1CC1(c2cccc(N3Cc4c(cc(CN5CCN6C(=O)CCC6C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)-isoindoline-5-carbaldehyde (100 mg, 0.22 mmol) and hexahydro-1H-pyrrolo[1,2-a]pyrazin-6-one (92 mg, 0.66 mmol) as reactants to afford the title compound (26 mg, 19%) as a white solid. ¹H NMR (500 MHz, Acetonitrile-d₃) δ 8.03 (s, 1H), 8.00-7.89 (m, 3H), 7.38 (dd, J=8.0 Hz, 1H), 7.34 (dd, J=2.0 Hz, 1H), 6.80 (dd, J=7.7, 1.5 Hz, 1H), 5.05 (d, J=6.0 Hz, 2H), 5.02-4.96 (m, 4H), 3.88 (dd, J=12.0, 3.2 Hz, 1H), 3.80-3.70 (m, 2H), 3.63 (dtd, J=10.8, 7.3, 3.7 Hz, 2H), 3.55 (s, 2H), 3.01-2.95 (m, 1H), 2.88 (s, 3H), 2.87-2.78 (m, 2H), 2.33-2.20 (m, 1H), 2.03 (td, J=12.1, 11.5, 3.7 Hz, 1H), 1.85 (t, J=10.8 Hz, 1H), 1.63-1.53 (m, 1H); LCMS: C₃₀H₃₁F3N₆O₃ requires: 580, found: m/z=581 [M+H]⁺.

### Example 712: 6-((2,6-dioxa-9-azaspiro[4.5]decan-9-yl)methyl)-2-(3-(3-((4-methyl-4H-1,2,4-triazol-3-yl)methyl)oxetan-3-yl)phenyl)-4-(trifluoromethyl)isoindolin-1-one

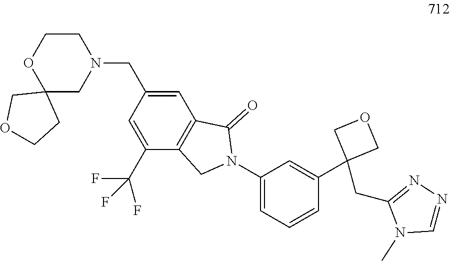 [SMILES: Cn1cnnc1CC1(c2cccc(N3Cc4c(cc(CN5CCOC6(CCOC6)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)isoindoline-5-carbaldehyde (100 mg, 0.22 mmol) and 2,6-dioxa-9-azaspiro[4.5]decane (94 mg, 0.66 mmol) as reactants to afford the title compound (39 mg, 26%) as a white solid. ¹H NMR (500 MHz, Acetonitrile-d₃) δ 8.02 (s, 1H), 7.98-7.88 (m, 3H), 7.43-7.30 (m, 2H), 6.79 (d, J=7.7 Hz, 1H), 5.05 (dd, J=6.1, 1.6 Hz, 2H), 5.03-4.96 (m, 4H), 3.88-3.60 (m, 8H), 3.55 (s, 2H), 2.89 (s, 3H), 2.51-2.41 (m, 4H), 2.09-1.99 (m, 1H), 1.94-1.83 (m, 1H); LCMS: C₃₀H₃₂F₃N₅O₄ requires: 583, found: m/z=584 [M+H]⁺.

### Example 713: rel-6-[(4aR,8aS)-octahydropyrido[3,4-b][1,4]oxazin-6-ylmethyl]-2-(3-{3-[(4-methyl-1,2,4-triazol-3-yl)methyl]oxetan-3-yl}phenyl)-4-(trifluoromethyl)-3H-isoindol-1-one

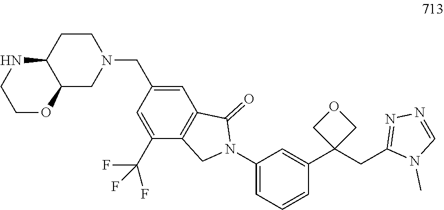 [SMILES: Cn1cnnc1CC1(c2cccc(N3Cc4c(cc(CN5CC[C@@H]6NCCO[C@@H]6C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)isoindoline-5-carbaldehyde (100 mg, 0.22 mmol) and rel-(4aR,8aS)-octahydro-1H-pyrido[3,4-b][1,4]oxazine (93 mg, 0.66 mmol) as reactants to afford the title compound (34 mg, 26%) as a white solid. ¹H NMR (500 MHz, Acetonitrile-d₃) δ 8.00 (s, 1H), 7.96-7.88 (m, 3H), 7.42-7.30 (m, 2H), 6.79 (d, J=7.7, 1.2 Hz, 1H), 5.04 (d, J=6.1 Hz, 2H), 5.02-4.98 (m, 4H), 3.77 (dt, J=11.3, 3.6 Hz, 1H), 3.73-3.63 (m, 3H), 3.55 (s, 2H), 3.48 (t, J=11.3 Hz, 1H), 3.06-2.94 (m, 1H), 2.89 (s, 3H), 2.84 (dt, J=8.0, 3.7 Hz, 1H), 2.78-2.70 (m, 1H), 2.61-2.46 (m, 1H), 2.53 (s, 1H), 2.47-2.34 (m, 3H), 1.79-1.61 (m, 1H); LCMS: C₃₀H₃₃F₃N₆O₄ requires: 582, found: m/z=583 [M+H]⁺.

### Example 714: 6-((2-(fluoromethyl)morpholino)methyl)-2-(3-(3-((4-methyl-4H-1,2,4-triazol-3-yl)methyl)oxetan-3-yl)phenyl)-4-(trifluoromethyl)isoindolin-1-one

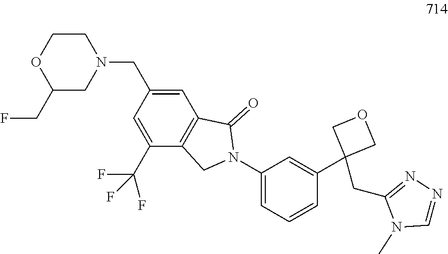 [SMILES: Cn1cnnc1CC1(c2cccc(N3Cc4c(cc(CN5CCOC(CF)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)-isoindoline-5-carbaldehyde (100 mg, 0.22 mmol) and 2-(fluoromethyl)morpholine (78 mg, 0.66 mmol) as reactants to afford the title compound (50 mg, 36%) as a white solid. ¹H NMR (500 MHz, Acetonitrile-d₃) δ 8.02 (s, 1H), 7.97-7.87 (m, 3H), 7.41-7.31 (m, 2H), 6.79 (d, J=7.7, 1.2 Hz, 1H), 5.04 (d, J=6.0 Hz, 2H), 4.99 (d, J=5.7 Hz, 4H), 4.40 (dd, J=47.5, 4.3 Hz, 2H), 3.89 (ddd, J=11.4, 3.3, 1.8 Hz, 1H), 3.84-3.62 (m, 4H), 3.55 (s, 2H), 2.89 (s, 3H), 2.77-2.67 (m, 2H), 2.24 (td, J=11.4, 3.3 Hz, 1H), 2.06 (t, J=10.8 Hz, 1H); LCMS: C₂₈H₂₉F₄N₅O₃ requires: 559, found: m/z=560 [M+H]⁺.

### Example 715: 2-(3-(3-((4-methyl-4H-1,2,4-triazol-3-yl)methyl)oxetan-3-yl)phenyl)-6-((2-(2,2,2-trifluoroethyl)morpholino)methyl)-4-(trifluoromethyl)isoindolin-1-one

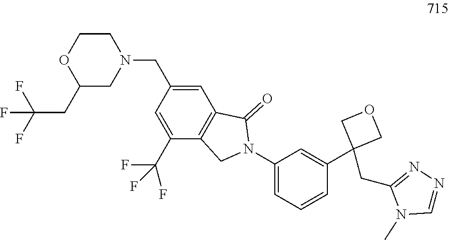 [SMILES: Cn1cnnc1CC1(c2cccc(N3Cc4c(cc(CN5CCOC(CC(F)(F)F)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)isoindoline-5-carbaldehyde (100 mg, 0.22 mmol) and 2-(2,2,2-trifluoroethyl)morpholine (111 mg, 0.66 mmol) as reactants to afford the title compound (45 mg, 33%) as a white solid. ¹H NMR (500 MHz, Acetonitrile-d₃) δ 8.02 (s, 1H), 7.98-7.87 (m, 3H), 7.41-7.32 (m, 2H), 6.79 (d, J=7.7, 1.2 Hz, 1H), 5.05 (d, J=6.1 Hz, 2H), 5.03-4.96 (m, 4H), 3.90-3.82 (m, 2H), 3.71 (s, 2H), 3.65 (td, J=11.2, 2.6 Hz, 1H), 3.55 (s, 2H), 2.89 (s, 3H), 2.78 (dt, J=11.3, 2.1 Hz, 1H), 2.71-2.64 (m, 1H), 2.47-2.22 (m, 3H), 2.04 (dd, J=11.3, 9.8 Hz, 1H); LCMS: C₂₉H₂₉F₆N₅O₃ requires: 609, found: m/z=610 [M+H]⁺.

### Example 716: 6-[(2-isopropylmorpholin-4-yl)methyl]-2-(3-{3-[(4-methyl-1,2,4-triazol-3-yl)methyl]oxetan-3-yl}phenyl)-4-(trifluoromethyl)-3H-isoindol-1-one

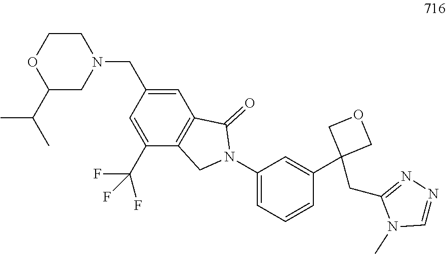 [SMILES: CC(C)C1CN(Cc2cc3c(c(C(F)(F)F)c2)CN(c2cccc(C4(Cc5nncn5C)COC4)c2)C3=O)CCO1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)isoindoline-5-carbaldehyde (100 mg, 0.22 mmol) and 2-isopropylmorpholine (85 mg, 0.66 mmol) as reactants to afford the title compound (36 mg, 29%) as a white solid. ¹H NMR (500 MHz, Acetonitrile-d₃) δ 8.01 (s, 1H), 7.96-7.88 (m, 3H), 7.38 (dd, J=7.9 Hz, 1H), 7.34 (dd, J=2.0 Hz, 1H), 6.79 (d, J=7.7, 1.2 Hz, 1H), 5.05 (d, J=6.0 Hz, 2H), 5.03-4.97 (m, 4H), 3.83 (ddd, J=11.3, 3.4, 1.6 Hz, 1H), 3.75-3.64 (m, 2H), 3.59 (dd, J=11.3, 2.5 Hz, 1H), 3.55 (s, 2H), 3.20 (ddd, J=9.5, 6.6, 2.2 Hz, 1H), 2.89 (s, 3H), 2.80 (dt, J=11.1, 2.0 Hz, 1H), 2.63 (dq, J=11.3, 2.0 Hz, 1H), 2.14 (d, J=8.0 Hz, 1H), 1.66 (h, J=6.8 Hz, 1H), 0.91 (dd, J=33.2, 6.8 Hz, 6H); LCMS: C₃₀H₃₄F₃N₅O₃ requires: 569, found: m/z=570 [M+H]⁺.

### Example 717: 6-{[2-(2-hydroxyethyl)morpholin-4-yl]methyl}-2-(3-{3-[(4-methyl-1,2,4-triazol-3-yl)methyl]oxetan-3-yl}phenyl)-4-(trifluoromethyl)-3H-isoindol-1-one

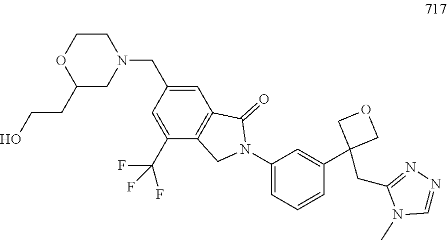 [SMILES: Cn1cnnc1CC1(c2cccc(N3Cc4c(cc(CN5CCOC(CCO)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)-isoindoline-5-carbaldehyde (100 mg, 0.22 mmol) and 2-(morpholin-2-yl)ethanol (86 mg, 0.66 mmol) as reactants to afford the title compound (19 mg, 15%) as a white solid. ¹H NMR (500 MHz, Acetonitrile-d₃) δ 8.01 (s, 1H), 7.96-7.89 (m, 3H), 7.38 (dd, J=8.0 Hz, 1H), 7.34 (dd, J=2.0 Hz, 1H), 6.79 (d, J=7.7, 1.3 Hz, 1H), 5.05 (d, J=6.1 Hz, 2H), 5.02-4.97 (m, 4H), 3.88-3.78 (m, 1H), 3.68 (s, 2H), 3.68-3.57 (m, 3H), 3.55 (s, 2H), 2.89 (s, 3H), 2.78-2.60 (m, 3H), 2.28-2.17 (m, 1H), 2.15-2.10 (m, 1H), 1.69-1.51 (m, 2H); LCMS: C₂₉H₃₂F₃N₅O₄ requires: 571, found: m/z=572 [M+H]⁺.

### Example 718: 6-[(2-cyclopropylmorpholin-4-yl)methyl]-2-(3-{3-[(4-methyl-1,2,4-triazol-3-yl)methyl]oxetan-3-yl}phenyl)-4-(trifluoromethyl)-3H-isoindol-1-one

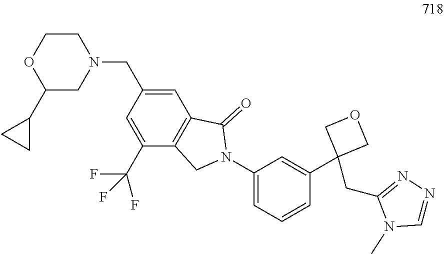 [SMILES: Cn1cnnc1CC1(c2cccc(N3Cc4c(cc(CN5CCOC(C6CC6)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)-isoindoline-5-carbaldehyde (100 mg, 0.22 mmol) and 2-cyclopropylmorpholine hydrochloride (108 mg, 0.66 mmol) as reactants to afford the title compound (31 mg, 24%) as a white solid. ¹H NMR (500 MHz, Acetonitrile-d₃) δ 8.00 (s, 1H), 7.95-7.87 (m, 3H), 7.38 (dd, J=8.0 Hz, 1H), 7.34 (dd, J=2.0 Hz, 1H), 6.79 (d, J=7.7, 1.3 Hz, 1H), 5.05 (d, J=6.0 Hz, 2H), 4.99 (d, J=6.2 Hz, 4H), 3.82 (ddd, J=11.3, 3.3, 1.8 Hz, 1H), 3.69 (s, 2H), 3.55 (s, 2H), 3.53 (d, J=2.6 Hz, 1H), 2.89 (s, 3H), 2.85-2.76 (m, 3H), 2.63 (dq, J=11.3, 2.0 Hz, 1H), 2.20 (dd, J=11.3, 3.3 Hz, 1H), 2.08 (dd, J=11.0, 9.7 Hz, 1H), 0.89-0.79 (m, 1H), 0.51-0.38 (m, 1H), 0.36-0.27 (m, 1H), 0.23-0.14 (m, 1H); LCMS: C₃₀H₃₂F₃N₅O₃ requires: 567, found: m/z=568 [M+H]⁺.

### Example 719: 6-[(2-cyclobutylmorpholin-4-yl)methyl]-2-(3-{3-[(4-methyl-1,2,4-triazol-3-yl)methyl]oxetan-3-yl}phenyl)-4-(trifluoromethyl)-3H-isoindol-1-one

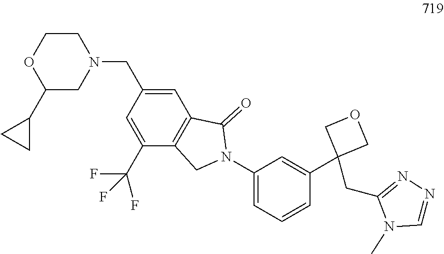 [SMILES: Cn1cnnc1CC1(c2cccc(N3Cc4c(cc(CN5CCOC(C6CC6)C5)cc4C(F)(F)F)C3=O)c2)COC1]

The reductive amination was carried out in a similar fashion as for 411, step 2 using 2-(3-(3-((4-methyl-4H-1,2,4-triazol-3-yl)methyl)oxetan-3-yl)phenyl)-3-oxo-7-(trifluoromethyl)-isoindoline-5-carbaldehyde (100 mg, 0.22 mmol) and 2-cyclobutylmorpholine (93 mg, 0.66 mmol) as reactants to afford the title compound (62 mg, 48%) as a white solid. ¹H NMR (500 MHz, Acetonitrile-d₃) δ 8.00 (s, 1H), 7.96-7.88 (m, 4H), 7.38 (dd, J=8.0 Hz, 1H), 7.34 (dd, J=2.0 Hz, 1H), 6.82-6.77 (m, 1H), 5.05 (d, J=6.0 Hz, 2H), 5.03-4.95 (m, 4H), 3.82 (ddd, J=11.4, 3.3, 1.7 Hz, 1H), 3.67 (d, J=2.0 Hz, 2H), 3.59 (td, J=11.3, 2.5 Hz, 1H), 3.55 (s, 2H), 3.42 (ddd, J=9.9, 7.5, 2.3 Hz, 1H), 2.89 (s, 3H), 2.72-2.67 (m, 1H), 2.67-2.63 (m, 1H), 2.40-2.30 (m, 1H), 2.15-2.11 (m, 1H), 1.95-1.72 (m, 6H); LCMS: C₃₁H₃₄F₃N₅O₃ requires: 581, found: m/z=582 [M+H]⁺.

### BIOLOGICAL EXAMPLES

### Biological Example 1: Evaluation of Cbl-b Inhibition by Cbl-b Inhibitor Candidates

Candidate compounds were isolated and evaluated for their ability to bind and inhibit Cbl-b, an E3 ubiquitin-protein ligase.

Materials and Methods

Cbl-b Activity Assay (Cbl-b Inhibition Assay)

The ability of candidate compounds to inhibit Cbl-b activity was measured by monitoring the interaction of Cbl-b with UbcH5B-Ub in the presence of the candidate compound. A truncated variant of Cbl-b (UniProt number Q13191; SEQ ID NO: 1) containing an Avitag at its N-terminus was co-expressed with BirA biotin ligase and purified using a standard protocol (see Dou et al., Nature Structural and Molecular Biology 8: 982-987, 2013; Avidity LLC).

Cbl-b amino acid residues 36-427
(SEQ ID NO: 1)
PKQAAADRRTVEKTWKLMDKVVRLCQNPKLQLKNSPPYILDILPDTYQHL
RLILSKYDDNQKLAQLSENEYFKIYIDSLMKKSKRAIRLFKEGKERMYEE
QSQDRRNLTKLSLIFSHMLAEIKAIFPNGQFQGDNFRITKADAAEFWRKF
FGDKTIVPWKVFRQCLHEVHQISSGLEAMALKSTIDLTCNDYISVFEFDI
FTRLFQPWGSILRNWNFLAVTHPGYMAFLTYDEVKARLQKYSTKPGSYIF
RLSCTRLGQWAIGYVTGDGNILQTIPHNKPLFQALIDGSREGFYLYPDGR
SYNPDLTGLCEPTPHDHIKVTQEQYELYCEMGSTFQLCKICAENDKDVKI
EPCGHLMCTSCLTAWQESDGQGCPFCRCEIKGTEPIIVDPFD

Fluorescently labeled UbcH5B-Ub was prepared by conjugating ubiquitin (Ub), labeled at its N-terminus with Bodipy-Fluorescein, to UbcH5B, an E2 enzyme, harboring a cysteine to lysine mutation at position 85. This mutation is similar to a mutation that was previously reported (see Dou et al., Nature Structural and Molecular Biology 8: 982-987, 2013). Cbl-b activity assays were performed in a 384-well plate at room temperature in a 10 pi reaction volume by pre-incubating 12 nM Cbl-b (final concentration) in an assay buffer containing 50 mM HEPES pH 7.0, 100 mM NaCl, 0.01% Triton X-100, 0.01% BSA and ImM DTT in the presence of a candidate compound in 1% DMSO (final concentration) for 1 hour. After incubation in the presence of the candidate compound, the plate was incubated for an additional 1.5 hours in the presence of 60 nM Src kinase with 1 mM ATP and 5 mM MgCl₂ (final concentrations). Src kinase was prepared as previously described (See Kobashigawa et al., PNAS 108(51): 20579-20584, 2011). Following incubation, 2 μl of a mixture containing 1.5 μM fluorescently labeled UbcH5B-Ub, 15 nM Streptavidin-Terbium (Cisbio), 300 nM EDTA and 0.01% BSA was added to the reaction, wherein the EDTA quenched the activity of the Src kinase. The plate was incubated for 1 hour. Following the 1 hour incubation, the plates were read for TR-FRET signal at 520/620 nM using Envision plate reader (Perkin Elmer). The presence of a FRET signal that indicated Cbl-b was not inhibited by the compound candidate (FIG. 1A). The absence of a FRET signal indicated Cbl-b was inhibited by the compound candidate and was therefore a Cbl-b inhibitor (FIG. 1B).

Cbl-b Binding Assay

SPR data was collected on a Biacore T200 (GE Healthcare) using a NeutrAvidin (Invitrogen) capture surface that was generated on a polycarboxylate hydrogel sensor chip of medium charge density (XanTec Bioanalytics GmbH) at 20° C. The surface was prepared using 20 mM HEPES, pH 7.5, 100 mM NaCl, 0.5 mM TCEP, 0.01% Triton X-100 as the immobilization buffer. Neutravidin was coupled to the sensor chip through EDC/NHS amine coupling. All steps were performed using a flow rate of 10 μl/min. First, the chip was preconditioned with a 3-min injection of 1 M NaCl, 0.1 M Borate followed by a 10-min equilibration in immobilization buffer. The surface was activated by a 5-min injection of 100 mM EDC/50 mM NHS. Next, 250 μg/mL NeutrAvidin buffered in 10 mM sodium acetate buffer, pH 5.0 was injected over the surface for 2 min. The surface was deactivated with 1 M Tris, pH 8.5 for 7 min then re-equilibrated in immobilization buffer for 7 min. 0.5 μM of N-terminal biotinylated Avi-tagged Cbl-b (residues 36-427) in immobilization buffer was captured on the NeutrAvidin surface to a density of 3500 RU. Flow cell 1 served as a reference surface and was prepared without protein. All binding measurements were made in 20 mM HEPES, pH 7.5, 100 mM NaCl, 0.5 mM TCEP, 0.01% Triton X-100, 2% DMSO (referred as assay buffer) at 20° C. Compounds, prepared in assay buffer, were diluted from atop concentration of either 100 μM or 50 μM using either a 2- or 3-fold dilution scheme containing 8 or 10 points. Samples were injected for 50 s at 50 μl/min. Dissociation time varied from 90-180 s, depending on anticipated potency. The surface was regenerated by injection of 0.3 M NaCl, prepared in assay buffer for 20 s at 10 μl/min. Positive and negative control samples were run between each compound to monitor surface integrity.

Results

The resulting data for the Cbl-b activity assays were analyzed using standard methods to report the IC₅₀ values of the tested compounds (Table 2). Column marked with an asterisk (*) were tested as described above with assay buffer containing 50 mM NaCl instead of 100 mM NaCl and 30 nM Src kinase instead of 60 nM Src kinase. The resulting data for the Cbl-b binding assay was solvent corrected and double referenced prior to analysis. All data were globally fit to a steady-state affinity and kinetic binding model where applicable). Fit parameters were derived from a 1:1 binding model using the Biacore T200 Evaluation Software V2.0 (GE Healthcare) (Table 2). Compounds were ranked into bins A through F as follows for both IC₅₀ and K_{D}: A indicates <100 nM, B indicates 100 nM-300 nM, C indicates 301 nM-1000 nM, D indicates 1,001 nM-3,000 nM, E indicates 3,001 nM-10,000 nM, F indicates >10,000 nM.

| Compound | Cbl-b activity | Cbl-b activity | Cbl-b binding |
|---|---|---|---|
| Identificaton No. | IC₅₀ (μM) | IC₅₀ (μM)* | K_{D} (μM) |
| 15 | | F | |
| 16 | | E | |
| 17 | | E | E |
| 18 | | E | |
| 19 | | E | |
| 20 | | E | |
| 21 | | F | |
| 22 | | F | |
| 23 | D | D | E |
| 24 | | E | |
| 25 | | D | |
| 26 | F | | |
| 27 | | E | |
| 28 | | D | |
| 29 | | E | F |
| 30 | C | C | D |
| 31 | | E | F |
| 32 | E | D | D |
| 33 | B | C | C |
| 34 | C | C | D |
| 35 | C | C | E |
| 36 | | D | |
| 37 | | E | |
| 38 | C | C | |
| 39 | B | C | |
| 40 | D | E | E |
| 41 | D | | D |
| 42 | C | | C |
| 43 | E | | F |
| 44 | E | | E |
| 45 | E | | E |
| 46 | D | | E |
| 47 | E | | E |
| 48 | C | | D |
| 49 | D | | D |
| 50 | E | | F |
| 51 | B | | C |
| 52 | E | | F |
| 53 | E | | E |
| 54 | A | | B |
| 55 | C | | D |
| 56 | C | | D |
| 57b | D | D | E |
| 57a | B | B | B |
| 58 | E | | |
| 59 | D | | E |
| 60 | D | | E |
| 61 | B | | B |
| 62 | B | | C |
| 63 | E | | |
| 64 | C | | D |
| 65 | A | | B |
| 66 | D | | E |
| 67 | A | | B |
| 68 | B | | C |
| 69 | C | | E |
| 70 | B | | C |
| 71 | B | | B |
| 72 | A | | B |
| 73 | D | | E |
| 74 | C | | D |
| 75 | C | | C |
| 76 | A | | B |
| 77 | C | | D |
| 78 | B | | B |
| 79 | E | | E |
| 80 | D | | E |
| 81 | B | | C |
| 82 | D | | E |
| 83 | D | | E |
| 84 | C | | |
| 85 | B | | C |
| 86 | E | | F |
| 87 | D | | D |
| 88 | D | | E |
| 89 | A | | B |
| 90 | D | | E |
| 91 | A | | B |
| 92 | A | | B |
| 93 | A | | B |
| 94 | D | | |
| 95a | C | | D |
| 95 | D | | D |
| 95b | E | | F |
| 96a | D | | D |
| 96 | | E | F |
| 97 | D | D | E |
| 98 | C | D | E |
| 99 | C | E | E |
| 100 | | E | |
| 101 | | E | |
| 102b | D | D | D |
| 102a | | E | |
| 103 | C | | D |
| 104 | C | | D |
| 105 | C | | D |
| 106 | D | | |
| 107 | D | | E |
| 108 | D | | E |
| 109 | E | | E |
| 110 | C | | E |
| 111 | E | | E |
| 112 | C | D | E |
| 113 | C | | D |
| 114 | E | | F |
| 115 | E | | |
| 116 | E | | E |
| 117 | D | | E |
| 118 | D | | |
| 119 | C | | |
| 120 | B | | C |
| 121 | E | | E |
| 122 | D | | D |
| 123 | D | | E |
| 124 | D | | E |
| 125 | D | | E |
| 126 | D | | E |
| 127 | E | | |
| 128a | D | | |
| 129 | F | | |
| 130a | C | | C |
| 131 | E | | |
| 132 | | F | |
| 133 | | E | E |
| 134 | D | D | F |
| 135 | D | E | F |
| 136 | | E | E |
| 137 | | E | E |
| 138 | E | | F |
| 139 | E | | E |
| 140 | B | | C |
| 141 | E | | E |
| 142 | F | | |
| 143 | E | | E |
| 144 | E | | E |
| 145 | D | | E |
| 146 | E | | E |
| 147 | E | | E |
| 148 | C | | D |
| 149 | E | | F |
| 150 | E | | F |
| 151 | C | | D |
| 152 | E | | E |
| 153 | E | | F |
| 154 | E | | F |
| 155 | D | | E |
| 156 | E | | E |
| 157 | E | | E |
| 158 | E | | |
| 159 | E | | E |
| 160 | E | | E |
| 161 | E | | F |
| 162 | | E | |
| 163 | E | E | E |
| 164 | C | | D |
| 165 | E | | |
| 166a | B | | C |
| 166b | D | | E |
| 166 | | C | |
| 167 | C | | C |
| 168a | B | | C |
| 168 | C | | D |
| 168b | D | | D |
| 169 | D | | D |
| 170 | F | | |
| 171 | | E | |
| 172a | C | | D |
| 172b | E | | F |
| 173 | E | | |
| 174 | D | | |
| 175 | E | | E |
| 176 | C | | D |
| 177 | E | | E |
| 178 | C | | C |
| 179 | E | | E |
| 180 | D | | D |
| 181 | D | | E |
| 182 | C | | C |
| 183a | A | | B |
| 183b | B | | E |
| 184 | D | | E |
| 185 | C | | D |
| 186 | C | | C |
| 187 | B | | B |
| 188 | B | | C |
| 189 | B | | C |
| 190 | B | | B |
| 191 | A | | A |
| 192 | C | | |
| 193 | A | | A |
| 194 | A | | B |
| 195 | A | | A |
| 196 | A | | B |
| 197 | D | | |
| 198 | B | | B |
| 198b | E | | E |
| 199 | A | | B |
| 200 | B | | C |
| 201 | B | | C |
| 202 | C | | |
| 203 | A | | A |
| 204 | D | | D |
| 205 | A | | B |
| 206 | C | | C |
| 207 | C | | D |
| 208 | C | | E |
| 209 | A | | |
| 210 | E | | E |
| 211 | A | | B |
| 212 | C | | D |
| 213a | B | | B |
| 213b | E | | |
| 214 | D | | |
| 215 | C | | |
| 216 | E | | |
| 217 | F | | |
| 218 | C | | |
| 219 | E | | |
| 220 | A | | A |
| 221 | C | | |
| 222 | C | | D |
| 223 | E | | E |
| 224 | B | | C |
| 225 | D | | E |
| 226 | E | | E |
| 227 | D | | E |
| 228 | E | | |
| 229 | D | | D |
| 230 | E | | |
| 231 | D | | B |
| 232 | D | | |
| 233 | E | | E |
| 234 | D | | D |
| 235 | D | | E |
| 236 | D | | D |
| 237 | E | | |
| 238 | D | | D |
| 239 | D | | B |
| 240 | B | | B |
| 241 | E | | A |
| 242 | E | | E |
| 243 | C | | F |
| 244 | D | | D |
| 245 | D | | E |
| 246 | D | | D |
| 247 | E | | E |
| 248 | E | | E |
| 249 | D | | D |
| 250 | D | | D |
| 251 | C | | D |
| 252 | E | | D |
| 252a | D | | E |
| 253 | E | | E |
| 254 | D | | E |
| 255a | A | | B |
| 255b | D | | E |
| 256 | B | | B |
| 257 | B | | C |
| 258 | B | | E |
| 259 | C | | D |
| 260 | A | | |
| 261 | E | | |
| 262 | C | | C |
| 263 | E | | E |
| 264 | E | | F |
| 265 | C | | E |
| 266 | E | | F |
| 267a | D | | E |
| 267 | E | | |
| 268 | C | | |
| 269 | D | | |
| 270a | C | | |
| 271 | E | | E |
| 271b | D | | |
| 271a | F | | |
| 272 | C | | E |
| 272a | B | | |
| 273 | D | | E |
| 274 | A | | A |
| 275 | A | | A |
| 276 | A | | C |
| 277 | A | | B |
| 278 | A | | |
| 279 | A | | |
| 280 | E | | D |
| 281a | B | | B |
| 281b | E | | B |
| 281 | B | | |
| 282 | C | | C |
| 283 | C | | |
| 283b | C | | C |
| 284 | A | | |
| 285 | B | | |
| 286 | B | | C |
| 287 | B | | D |
| 288 | E | | |
| 289 | C | | |
| 290 | D | | |
| 291 | E | | |
| 292 | D | | |
| 293 | B | | |
| 294 | C | | A |
| 295 | E | | D |
| 296 | C | | E |
| 296a | B | | |
| 296b | E | | |
| 297 | E | | |
| 298 | B | | A |
| 298b | B | | B |
| 298a | D | | E |
| 299 | A | | D |
| 300 | A | | B |
| 301 | A | | C |
| 302 | C | | D |
| 303 | C | | E |
| 304b | C | | D |
| 304a | E | | F |
| 305 | C | | F |
| 306a | B | | B |
| 306b | E | | E |
| 307 | B | | C |
| 308 | D | | E |
| 309 | D | | E |
| 310 | C | | C |
| 311 | B | | B |
| 312a | B | | C |
| 312b | D | | |
| 313 | B | | C |
| 314 | C | | |
| 315 | C | | |
| 316 | C | | |
| 317 | C | | |
| 318 | C | | |
| 319 | B | | B |
| 320 | B | | B |
| 321 | A | | B |
| 322 | C | | |
| 323 | B | | B |
| 324 | C | | |
| 325 | B | | C |
| 326 | B | | B |
| 327 | C | | D |
| 328 | A | | |
| 329 | B | | |
| 330 | A | | C |
| 331 | C | | D |
| 332 | B | | C |
| 333 | B | | |
| 334 | B | | |
| 335 | A | | A |
| 336 | A | | A |
| 337 | B | | |
| 338 | A | | |
| 339 | A | | |
| 340 | A | | |
| 341 | A | | |
| 342 | A | | |
| 343 | A | | |
| 344 | B | | A |
| 345 | B | | |
| 346 | A | | |
| 347 | A | | |
| 348 | A | | |
| 349 | C | | |
| 350 | C | | |
| 351 | B | | |
| 352 | A | | B |
| 353 | A | | A |
| 354 | B | | |
| 355 | C | | |
| 356 | A | | |
| 357 | B | | B |
| 358 | A | | A |
| 359 | C | | |
| 360 | A | | A |
| 361 | C | | C |
| 362 | B | | B |
| 363 | A | | A |
| 364 | C | | D |
| 365 | B | | |
| 366 | B | | B |
| 367b | D | | |
| 367a | B | | D |
| 368 | C | | |
| 369 | C | | |
| 370 | B | | B |
| 371 | B | | C |
| 372 | C | | |
| 373 | C | | |
| 374 | A | | B |
| 375 | A | | A |
| 376 | B | | |
| 377 | A | | A |
| 378 | A | | A |
| 379 | A | | A |
| 380 | A | | A |
| 381 | A | | A |
| 382 | B | | |
| 383 | A | | B |
| 384 | A | | A |
| 385 | A | | A |
| 386 | A | | |
| 387 | A | | |
| 388 | A | | |
| 389 | A | | |
| 390 | A | | |
| 391 | A | | |
| 392 | A | | A |
| 393 | A | | A |
| 394 | A | | A |
| 395 | A | | A |
| 396 | A | | A |
| 397 | A | | A |
| 398 | A | | A |
| 399 | A | | A |
| 400 | A | | A |
| 401 | A | | A |
| 402 | A | | A |
| 403 | A | | A |
| 404 | A | | |
| 405 | A | | |
| 406 | A | | |
| 407 | A | | A |
| 408 | B | | B |
| 409 | A | | |
| 410 | A | | |
| 411 | A | | A |
| 412 | A | | |
| 413 | A | | |
| 414 | A | | |
| 415 | A | | |
| 416 | A | | A |
| 417 | A | | A |
| 418 | A | | A |
| 419 | A | | |
| 420 | A | | |
| 421 | A | | |
| 422 | A | | |
| 423 | A | | |
| 424 | A | | |
| 425 | A | | |
| 426 | A | | |
| 427 | A | | |
| 428 | A | | |
| 429 | A | | |
| 430 | A | | |
| 431 | A | | |
| 432 | A | | |
| 433 | A | | |
| 434 | A | | |
| 435 | A | | |
| 436 | A | | A |
| 437 | A | | |
| 438 | A | | B |
| 439 | C | | |
| 440 | B | | |
| 441 | B | | |
| 442 | A | | |
| 443 | A | | |
| 444 | A | | |
| 445 | A | | |
| 446 | A | | B |
| 447 | A | | A |
| 448 | A | | A |
| 449 | C | | |
| 450 | A | | A |
| 451 | B | | A |
| 452 | A | | A |
| 453 | A | | |
| 454 | A | | |
| 455 | A | | |
| 456 | B | | |
| 457 | A | | A |
| 458 | A | | A |
| 459 | A | | |
| 460 | A | | |
| 461 | A | | |
| 462 | A | | |
| 463 | A | | A |
| 464 | A | | A |
| 465 | A | | A |
| 466 | A | | |
| 467a | B | | C |
| 468 | A | | |
| 469 | B | | |
| 470 | B | | |
| 471a | B | | C |
| 471b | E | | |
| 472 | A | | A |
| 473 | C | | |
| 474a | A | | B |
| 475 | A | | B |
| 476 | C | | |
| 477 | A | | A |
| 478b | D | | |
| 478a | C | | |
| 479 | A | | B |
| 480 | B | | |
| 481a | B | | B |
| 481b | D | | |
| 482 | C | | |
| 483 | B | | |
| 484 | B | | |
| 485 | B | | |
| 486a | A | | |
| 486b | A | | |
| 487 | A | | |
| 488 | A | | |
| 489 | A | | |
| 490 | A | | |
| 491 | A | | |
| 492 | A | | |
| 493 | C | | |
| 494 | B | | |
| 495 | C | | |
| 496 | A | | A |
| 497 | A | | |
| 498 | A | | |
| 499 | A | | |
| 500 | A | | |
| 501 | A | | |
| 502 | A | | |
| 503 | A | | |
| 504 | A | | |
| 505 | A | | |
| 506 | A | | A |
| 507 | A | | |
| 508 | A | | |
| 509 | A | | |
| 510 | A | | |
| 511 | A | | |
| 512 | A | | |
| 513 | A | | |
| 514 | A | | |
| 515 | A | | |
| 516 | A | | |
| 517a | A | | |
| 517b | A | | |
| 518 | B | | |
| 519 | A | | |
| 520 | A | | |
| 521 | A | | |
| 522 | B | | |
| 523 | B | | |
| 524 | A | | |
| 525 | A | | |
| 526 | A | | |
| 527 | A | | |
| 528 | A | | |
| 529 | A | | |
| 530 | A | | |
| 531 | A | | |
| 532 | A | | |
| 533 | A | | |
| 534a | B | | |
| 534b | A | | |
| 535 | A | | |
| 536 | A | | |
| 537 | | | |
| 538 | A | | |
| 539 | | | |
| 540 | A | | |
| 541a | A | | |
| 541b | A | | |
| 542 | A | | |
| 543a | A | | |
| 543b | A | | |
| 544 | C | | |
| 545 | A | | |
| 546 | A | | |
| 547 | A | | |
| 548 | B | | |
| 549 | B | | |
| 550 | A | | |
| 551 | A | | |
| 552 | A | | |
| 553a | A | | A |
| 553b | A | | |
| 554 | A | | A |
| 555 | A | | |
| 556 | A | | |
| 557 | A | | |
| 558a | A | | |
| 558b | A | | |
| 559 | B | | |
| 560 | A | | |
| 561 | A | | |
| 562 | A | | |
| 563 | A | | |
| 564 | A | | |
| 565 | A | | |
| 566 | A | | |
| 567 | A | | |
| 568 | A | | |
| 569 | A | | |
| 570 | A | | |
| 571 | A | | A |
| 572 | A | | |
| 573 | A | | |
| 574 | A | | |
| 575 | A | | |
| 576 | A | | A |
| 577 | B | | |
| 578 | A | | |
| 579 | A | | |
| 580 | A | | |
| 581 | A | | |
| 582 | A | | |
| 583 | A | | |
| 584 | A | | |
| 585 | A | | |
| 586 | A | | |
| 587 | A | | |
| 588 | A | | |
| 589 | A | | |
| 590 | A | | |
| 591 | A | | |
| 592a | A | | |
| 592b | A | | |
| 593 | A | | |
| 594 | A | | |
| 595 | A | | |
| 596 | B | | |
| 597 | B | | |
| 598 | B | | |
| 599 | C | | |
| 600 | A | | |
| 601 | A | | |
| 602 | A | | |
| 603 | A | | |
| 604 | A | | |
| 605 | B | | |
| 606 | A | | |
| 607 | A | | |
| 608 | A | | |
| 609 | A | | |
| 610 | A | | |
| 611 | A | | |
| 612 | A | | |
| 613 | A | | |
| 614 | A | | |
| 615 | A | | |
| 616 | A | | |
| 617 | B | | |
| 618 | A | | |
| 619 | A | | |
| 620 | A | | A |
| 621 | A | | |
| 622a | | | |
| 622b | | | |
| 623 | A | | |
| 624 | A | | |
| 625 | A | | |
| 626a | A | | |
| 626b | A | | |
| 627 | A | | |
| 628 | A | | |
| 629 | A | | |
| 630 | A | | |
| 631 | A | | |
| 632 | A | | |
| 633 | A | | |
| 634 | A | | |
| 635 | A | | |
| 636 | A | | |
| 637 | A | | |
| 638 | A | | |
| 639a | A | | |
| 639b | A | | |
| 640 | A | | |
| 641 | A | | |
| 642 | A | | |
| 643 | A | | |
| 644a | A | | |
| 644b | A | | |
| 645 | A | | |
| 646 | A | | |
| 647 | A | | |
| 648 | A | | |
| 649 | A | | |
| 650 | A | | |
| 651 | A | | |
| 652 | A | | |
| 653 | A | | |
| 654 | A | | |
| 655 | A | | |
| 656 | A | | |
| 657 | A | | |
| 658 | A | | |
| 659 | A | | |
| 660 | A | | |
| 661 | A | | |
| 662a | A | | |
| 662b | A | | |
| 663a | A | | |
| 663b | A | | |
| 664 | A | | |
| 665 | A | | |
| 666 | A | | |
| 667 | A | | |
| 668a | A | | |
| 668b | A | | |
| 669a | A | | |
| 669b | A | | |
| 670a | A | | A |
| 670b | A | | A |
| 671a | A | | |
| 671b | A | | |
| 672a | A | | |
| 672b | A | | |
| 673a | A | | |
| 673b | A | | |
| 674a | A | | |
| 674b | A | | |
| 675a | A | | |
| 675b | A | | |
| 676 | A | | |
| 677 | C | | |
| 678 | A | | |
| 679 | A | | |
| 680 | A | | |
| 681 | C | | |
| 682 | C | | |
| 683 | B | | |
| 684 | C | | D |
| 685 | C | | |
| 686 | C | | |
| 687 | C | | |
| 688 | C | | D |
| 689a | C | | |
| 689b | C | | |
| 690 | A | | |
| 691a | A | | |
| 691b | A | | |
| 692 | A | | |
| 693 | A | | |
| 694 | A | | |
| 695 | A | | |
| 696 | A | | |
| 697 | A | | |
| 698 | A | | |
| 699 | A | | |
| 700 | B | | |
| 701 | C | | |
| 702 | A | | |
| 703 | A | | |
| 704 | A | | |
| 705 | E | | |
| 706 | A | | |
| 707 | A | | |
| 708 | A | | |
| 709 | A | | |
| 710 | A | | |
| 711 | A | | |
| 712 | A | | |
| 713 | A | | |
| 714 | A | | |
| 715 | A | | |
| 716 | A | | |
| 717 | A | | |
| 718 | A | | |
| 719 | A | | |

Blank cell indicates data not available.

Conclusions

Compounds with the ability to inhibit Cbl-b activity were identified.

### Biological Example 2: Evaluation of T-Cell Activation by Cbl-b Inhibitors

Loss of Cbl-b function in both T-cells and mice by genetic knockout of the cbl-b gene results in loss of the CD28 co-stimulation requirement for T-cell activation and T-cell resistance to anergy (See Bachmaier et al., Nature, 403(6766):211-216, 2000; and Jeon et al., Immunity, 21(2): 167-177, 2004). Cbl-b inhibitors described herein were evaluated for their ability to activate T-cells.

Materials and Methods

Purification and Assessment of Primary Human Total T-Cell Activation

Peripheral blood mononuclear cells (PBMC) were obtained either: 1) by using Ficoll-Paque™ (GE Healthcare) for separation of peripheral blood hematopoietic cells from buffy coats of healthy human donors; or 2) directly from LeukoPak donations. Total human primary T-cells were isolated from the PMBCs utilizing negative selection with commercial kits following the manufacturer's protocol (Miltenyi Catalog #130-096-535 (i.e., cocktail of antibodies against surface markers CD14, CD15, CD16, CD19, CD34, CD36, CD56, CD123 and CD235a were incubated with the PBMCs before passing the samples by magnetic beads for removal of cells expressing those surface markers) or StemCells Catalog #17951) to yield >95% CD3+ cells as assessed by flow cytometry. The cells were rested overnight at 37° C. 5% CO₂. The Cbl-inhibitor was added to 1×10⁵ cells per well and the plate was incubated for one hour at 37° C. in 5% CO₂ at the concentrations indicated (Table 3) with a final DMSO concentration of <0.1%. For samples stimulated with anti-CD3 antibody and anti-CD28 antibody (anti-CD3/anti-CD28), the Cbl-b inhibitor concentrations tested were 4 μM, 1 μM, and 0.3 μM. For samples stimulated with anti-CD3 antibody alone (anti-CD3), the Cbl-b inhibitor concentrations tested were 8 μM, and 1 μM. Following incubation with the Cbl-b inhibitor, primary human total T-cells were stimulated with either plate bound anti-CD3 antibody (OKT3) alone or plate bound anti-CD3 antibody (OKT3) with soluble anti-CD28 antibody (28.2) (Life Technologies). To prepare plates with plate bound anti-CD3 antibody (OKT3), 96-well round bottom tissue culture plates were coated with 100 pi of anti-CD3 antibody (OKT3) at 10 μg/mL for 4 hours at 37° C. 5% CO₂ in phosphate buffered saline (PBS). The plates were washed with PBS once prior to adding the cells with or without soluble anti-CD28 antibody (28.2) to each well at a final concentration of 5 μg/mL. Cells were stimulated for 48 hours prior to harvesting the cell free supernatant and staining the cell population for surface marker assessment by flow cytometry. Supernatants were analyzed for cytokine secretion, including IL-2 by ELISA (R&D Systems, Peprotech or Life Technologies) or Luminex multiplex kits (Procarta Life Technologies) following the manufacturer's protocol. Cells were stained with anti-CD25 antibody (BD Biosciences) to assess levels of surface marker of activation.

Results

Readouts were reported as fold change over baseline. Baseline for this study was the measurement obtained from total human T-cells stimulated with anti-CD3 antibody and with soluble anti-CD28 antibody, wherein the cells were not incubated with a Cbl-b inhibitor (Table 3). For T-cells stimulated with anti-CD3/anti-CD28, changes greater than 2.5-fold over baseline for IL-2 secretion and greater than 1.3-fold over baseline for CD25 surface staining were considered significant and a positive response (Table 3). For T-cells stimulated with anti-CD3 alone, changes greater than 0.1-fold over baseline for IL-2 secretion and greater than 0.6-fold over baseline for CD25 surface staining were considered significant and a positive response (Table 3). Compounds were ranked into bins according to their readouts as follows:

For IL-2 secretion with anti-CD3 antibody and anti-CD28 antibody co-stimulation the bins are: E indicates ≤1.00 fold, D indicates 1.01-2.50 fold, C indicates 2.51-5.00 fold, B indicates 5.00-7.50 fold, and A indicates ≥7.50 fold;

For IL-2 secretion with anti-CD3 antibody stimulation the bins are: E indicates ≤0.050 fold, D indicates 0.051-0.1 fold, C indicates 0.101-0.15 fold, B indicates 0.151-0.2 fold, and A indicates ≥0.201 fold;

For CD25 staining with anti-CD3 antibody and anti-CD28 antibody co-stimulation the bins are: E indicates ≤1.00 fold, D indicates 1.01-1.30 fold, C indicates 1.31-1.50 fold, B indicates 1.51-1.75 fold, and A indicates ≥1.76 fold; and

For CD25 staining with anti-CD3 antibody stimulation the bins are: D indicates ≤0.600 fold, C indicates 0.601<0.800 fold, B indicates 0.801<1.00 fold, and A indicates ≥1.001 fold.

TABLE 3
T-cell activation as assessed by IL-2 secretion and CD25 surface staining

| | IL-2 secretion | | | IL-2 secretion | | CD25 staining | | | CD25 staining | |
|---|---|---|---|---|---|---|---|---|---|---|
| | Anti-CD3/Anti-CD28 | | | Anti-CD3 | | Anti-CD3/Anti-CD28 | | | Anti-CD3 | |
| Compound | stimulation | | | stimulation | | stimulation | | | stimulation | |
| ID No. | 4μM | 1 μM | 0.3 μM | 8 μM | 1 μM | 4 μM | 1 μM | 0.3 μM | 8 μM | 1 μM |
| 8a | D | D | D | D | E | D | D | D | D | D |
| 9 | C | D | D | D | E | D | E | E | C | D |
| 9a | D | D | D | D | E | D | D | D | C | C |
| 30 | C | D | D | C | D | D | D | D | B | C |
| 32 | D | D | E | C | E | D | D | D | A | C |
| 33 | A | C | C | E | E | D | D | D | B | D |
| 35 | C | C | D | A | E | C | D | E | C | D |
| 38 | C | D | D | B | C | C | E | E | C | D |
| 39 | A | B | C | D | E | D | D | E | B | D |
| 48 | A | B | C | C | E | D | D | D | B | B |
| 51 | D | E | E | E | E | D | D | D | A | B |
| 54 | A | C | D | B | E | D | D | D | B | C |
| 55 | D | D | D | E | E | D | D | D | C | D |
| 56 | A | B | C | E | E | D | D | D | B | D |
| 57b | D | D | D | D | D | D | D | D | C | D |
| 57a | A | C | D | A | D | A | C | C | A | C |
| 61 | D | E | E | E | E | D | D | E | A | C |
| 62 | A | B | C | D | E | D | D | E | B | D |
| 64 | D | E | E | E | E | D | E | E | C | D |
| 65 | C | D | D | C | E | D | D | D | A | C |
| 67 | C | D | D | C | E | D | D | D | A | B |
| 68 | D | D | D | D | E | D | D | D | B | C |
| 69 | D | E | E | E | E | C | D | D | D | D |
| 70 | C | D | E | D | E | B | C | C | B | B |
| 71 | B | C | C | A | D | A | B | D | B | B |
| 72 | C | C | D | B | D | B | B | C | B | D |
| 75 | D | D | D | D | E | D | D | D | B | C |
| 76 | B | C | E | D | E | C | D | D | A | C |
| 77 | D | D | E | E | E | D | D | E | C | D |
| 78 | C | D | D | A | E | D | D | E | B | C |
| 80 | D | D | E | E | E | D | D | D | D | D |
| 81 | C | E | E | B | E | A | B | D | A | B |
| 84 | E | E | E | E | E | D | D | E | C | D |
| 85 | D | E | E | E | E | D | D | D | A | C |
| 89 | B | C | D | C | E | C | D | D | B | D |
| 91 | A | C | D | C | E | D | E | E | A | C |
| 92 | B | C | D | C | E | C | C | D | A | C |
| 93 | C | D | D | C | E | D | D | D | A | C |
| 95a | C | D | D | D | E | D | D | E | D | D |
| 98 | D | D | D | D | D | E | E | E | D | D |
| 103 | D | D | D | D | E | D | E | E | B | D |
| 104 | D | D | D | E | E | D | E | E | B | C |
| 105 | E | D | D | E | E | E | D | D | C | D |
| 113 | B | C | C | E | E | D | E | E | D | D |
| 120 | D | D | D | E | E | E | E | E | D | D |
| 130a | A | B | C | A | E | A | B | C | A | C |
| 140 | E | E | E | E | E | D | D | D | B | C |
| 148 | E | E | E | E | E | E | E | E | C | D |
| 151 | A | A | A | E | E | D | D | D | C | D |
| 166a | D | E | E | E | E | D | D | E | B | C |
| 167 | C | E | D | C | E | A | D | B | C | D |
| 168a | E | D | E | E | E | D | D | D | B | D |
| 168 | D | D | D | D | E | D | E | E | C | B |
| 172a | C | C | C | E | E | E | E | E | D | D |
| 183a | A | B | C | A | A | A | A | A | A | A |
| 185 | C | D | D | D | D | A | A | D | D | D |
| 186 | B | D | C | C | D | A | C | D | D | D |
| 187 | A | C | D | A | A | A | A | B | A | B |
| 188 | E | E | E | D | E | A | C | D | D | D |
| 189 | B | C | D | D | D | A | A | A | A | C |
| 190 | B | D | E | C | E | A | A | A | A | C |
| 191 | A | B | C | A | A | B | B | C | A | A |
| 192 | D | D | D | C | C | D | D | D | B | B |
| 193 | A | B | C | A | A | B | B | D | A | A |
| 194 | A | A | C | A | A | A | B | D | A | B |
| 195 | A | A | B | A | A | A | A | B | A | A |
| 196 | A | B | C | A | A | A | B | D | A | A |
| 198 | D | D | D | A | C | E | D | D | A | B |
| 199 | A | B | C | A | A | B | C | D | A | A |
| 200 | D | E | E | D | E | D | D | D | A | B |
| 201 | E | D | D | A | B | D | D | D | A | B |
| 202 | C | D | D | B | D | C | D | D | B | D |
| 203 | A | A | A | A | A | B | B | C | A | A |
| 205 | B | C | C | C | D | C | D | D | A | B |
| 206 | C | D | D | D | E | D | D | D | B | B |
| 209 | A | B | B | A | A | B | C | C | A | D |
| 211 | A | B | C | A | A | B | D | D | A | C |
| 212 | D | D | D | E | E | A | B | B | D | D |
| 213a | C | D | D | C | D | D | D | D | A | B |
| 214 | A | A | A | D | D | D | E | E | D | D |
| 215 | D | E | E | D | D | B | C | B | D | D |
| 220 | A | A | C | A | A | A | B | C | A | A |
| 221 | E | E | E | D | D | C | C | D | D | D |
| 222 | E | E | E | E | E | B | C | B | D | D |
| 224 | E | E | E | C | C | B | D | D | D | D |
| 240 | C | D | E | A | B | C | D | E | B | C |
| 243 | C | E | E | A | A | C | E | E | B | C |
| 251 | D | E | E | B | B | D | D | D | C | C |
| 255a | B | C | D | A | D | A | B | C | A | C |
| 256 | A | B | C | A | A | B | C | D | B | B |
| 257 | D | D | D | A | B | C | D | D | A | C |
| 258 | B | C | D | C | D | D | D | E | A | A |
| 260 | C | D | D | A | A | C | D | D | A | B |
| 262 | D | E | E | E | E | C | C | D | D | D |
| 265 | D | D | E | E | E | D | D | D | B | C |
| 268 | D | D | D | D | E | D | D | D | B | C |
| 270a | D | D | D | B | C | D | D | D | B | C |
| 272 | E | E | E | C | C | D | E | E | D | D |
| 274 | A | C | D | A | B | C | D | D | A | B |
| 275 | A | B | C | A | A | A | B | C | A | B |
| 276 | A | C | D | B | D | C | D | D | A | B |
| 277 | C | C | D | C | D | C | D | D | A | B |
| 278 | C | D | D | A | B | D | D | D | A | B |
| 279 | A | B | C | A | A | C | C | D | A | A |
| 281a | C | D | D | D | E | C | D | D | A | B |
| 282 | C | D | E | E | E | A | A | A | A | C |
| 283 | D | D | E | C | E | D | E | E | D | D |
| 283b | A | A | B | A | C | D | D | D | B | C |
| 284 | A | C | D | A | A | C | D | D | A | B |
| 286 | C | D | D | C | D | D | E | E | A | B |
| 289 | D | E | E | B | C | D | D | D | B | B |
| 292 | D | D | C | E | E | D | E | D | D | D |
| 293 | C | C | C | A | C | D | D | D | B | C |
| 296 | D | E | E | A | A | D | D | D | C | C |
| 296a | C | D | D | A | B | D | D | D | A | C |
| 298 | A | B | B | C | D | D | D | E | C | C |
| 298b | D | D | D | C | D | D | E | D | B | C |
| 299 | B | C | C | A | D | D | D | E | A | A |
| 300 | A | C | D | A | A | B | D | D | A | B |
| 301 | A | C | D | C | D | C | D | D | A | B |
| 302 | A | C | C | D | E | D | D | D | B | D |
| 304b | D | E | E | E | E | C | D | D | D | D |
| 306a | D | D | E | E | E | D | D | D | B | D |
| 307 | C | D | D | C | D | D | E | E | D | D |
| 311 | D | E | E | E | E | D | E | E | C | C |
| 312a | C | D | D | B | D | D | D | E | B | C |
| 313 | C | D | D | B | C | D | D | D | B | C |
| 314 | D | D | D | C | D | D | D | D | C | C |
| 315 | D | D | E | C | D | D | D | D | B | C |
| 316 | C | D | D | B | D | D | D | D | A | C |
| 317 | D | D | D | C | D | D | D | D | B | C |
| 318 | D | D | D | C | D | D | D | E | B | C |
| 319 | C | D | D | A | D | D | D | D | B | C |
| 320 | B | C | D | A | C | D | D | D | A | B |
| 321 | C | C | D | A | D | D | D | D | A | B |
| 322 | D | D | D | C | D | D | D | D | C | C |
| 323 | B | C | D | A | C | D | E | E | A | C |
| 324 | D | D | D | D | D | D | E | E | C | C |
| 325 | C | D | D | C | D | D | D | D | A | B |
| 326 | C | C | D | A | D | D | D | D | A | B |
| 327 | D | D | D | D | D | D | D | D | B | C |
| 328 | C | D | D | C | D | D | D | D | A | B |
| 329 | B | D | D | A | C | C | D | D | A | C |
| 330 | B | C | D | A | D | D | D | D | A | B |
| 331 | D | D | D | D | E | D | E | E | C | D |
| 332 | C | D | D | A | C | D | D | D | B | C |
| 333 | B | C | D | A | C | D | D | D | A | B |
| 334 | B | C | D | A | C | D | D | D | B | C |
| 335 | A | B | C | A | C | B | B | C | A | B |
| 336 | A | A | B | A | A | B | A | C | A | A |
| 337 | C | C | D | B | D | D | D | D | A | C |
| 338 | A | C | D | A | C | D | D | D | A | B |
| 339 | A | A | B | A | A | B | C | C | A | A |
| 340 | A | B | D | A | B | C | D | D | A | B |
| 341 | A | A | C | A | B | B | C | D | A | B |
| 342 | | A | B | | B | | C | D | | A |
| 343 | | D | D | | D | | D | D | | C |
| 344 | A | C | D | A | C | C | C | D | A | B |
| 345 | C | D | D | B | D | D | D | D | C | C |
| 346 | | C | D | | C | | D | D | | C |
| 347 | A | A | C | A | B | C | D | D | A | A |
| 348 | | A | C | | A | | C | C | | B |
| 350 | D | D | E | D | D | D | D | D | B | C |
| 351 | D | D | D | B | E | D | D | D | A | C |
| 352 | B | D | D | A | D | C | D | D | A | B |
| 353 | A | C | D | A | C | C | D | D | A | B |
| 354 | B | D | D | A | C | C | D | D | A | C |
| 355 | D | D | D | C | D | D | D | D | C | C |
| 356 | A | C | C | A | B | C | D | D | A | B |
| 357 | C | D | D | B | D | D | D | D | A | C |
| 358 | A | B | C | A | B | C | C | D | A | A |
| 359 | C | D | D | A | D | C | D | D | A | C |
| 360 | A | A | C | A | A | B | B | C | A | A |
| 361 | D | D | D | B | D | D | D | E | B | C |
| 362 | D | D | D | C | D | D | D | E | B | C |
| 363 | B | D | D | B | D | C | D | D | A | C |
| 364 | D | D | D | C | D | D | D | D | C | C |
| 365 | C | D | D | B | D | D | D | D | A | C |
| 366 | B | C | D | A | D | C | D | D | A | B |
| 367a | D | E | E | B | D | D | E | D | B | C |
| 369 | D | D | D | C | D | D | D | D | B | C |
| 370 | B | D | D | A | D | A | D | D | B | C |
| 371 | B | C | D | A | C | D | D | D | A | B |
| 372 | C | D | D | C | D | D | D | D | B | C |
| 374 | A | B | C | A | B | C | C | D | A | B |
| 375 | A | B | C | A | A | C | C | D | A | A |
| 376 | A | C | D | A | C | B | C | D | A | B |
| 377 | A | B | D | A | A | A | B | C | B | C |
| 378 | A | A | C | A | A | A | B | C | A | A |
| 379 | C | C | D | B | C | D | D | D | A | B |
| 380 | A | C | C | A | B | B | C | C | A | B |
| 381 | A | B | C | A | B | B | B | C | A | B |
| 382 | B | B | C | A | C | B | C | D | A | C |
| 383 | A | B | C | A | A | B | B | C | A | B |
| 384 | A | A | C | A | A | B | B | C | A | A |
| 385 | A | C | D | A | C | C | D | D | A | A |
| 386 | | A | A | | A | | C | C | | A |
| 387 | | B | C | | A | | C | D | | B |
| 388 | | B | C | | B | | D | D | | A |
| 389 | | A | B | | A | | C | D | | A |
| 390 | | A | C | | B | | C | D | | A |
| 391 | | D | D | | D | | D | D | | B |
| 392 | A | B | C | A | B | C | D | D | A | A |
| 393 | A | A | C | A | A | C | C | D | A | A |
| 395 | A | A | C | A | A | C | C | D | A | A |
| 396 | A | B | C | A | B | C | D | D | A | A |
| 397 | A | A | C | A | B | C | C | D | A | A |
| 398 | A | B | C | A | C | C | C | D | A | A |
| 399 | A | A | B | A | B | C | C | D | A | A |
| 400 | A | A | B | A | A | C | D | D | A | A |
| 401 | A | A | C | A | B | C | D | D | A | A |
| 402 | A | A | B | A | B | C | C | D | A | A |
| 403 | A | A | B | A | B | C | D | D | A | A |
| 404 | A | A | A | A | A | C | C | C | A | A |
| 405 | A | B | C | A | A | C | C | D | A | B |
| 406 | A | A | B | A | A | C | C | D | A | A |
| 407 | A | B | C | A | C | D | D | D | A | A |
| 408 | C | D | D | C | E | D | D | D | B | C |
| 409 | A | A | C | A | A | B | C | C | A | A |
| 410 | A | A | B | A | A | B | B | C | A | A |
| 411 | A | A | C | A | A | D | D | D | A | A |
| 412 | A | A | A | A | A | B | B | C | A | A |
| 413 | A | A | B | A | A | C | B | C | A | A |
| 414 | A | A | A | A | B | B | C | C | A | B |
| 415 | A | A | B | A | B | B | C | C | A | A |
| 416 | A | A | C | A | B | B | C | C | A | A |
| 417 | A | B | C | A | C | C | C | D | A | A |
| 418 | A | B | C | A | B | C | C | D | A | A |
| 419 | A | A | C | A | B | C | C | D | A | A |
| 420 | A | A | C | A | B | C | C | C | A | A |
| 421 | A | A | B | A | C | C | D | D | A | A |
| 422 | A | A | B | A | A | C | C | C | A | A |
| 423 | A | B | C | A | B | C | D | D | A | B |
| 424 | A | A | A | A | A | B | B | C | A | A |
| 425 | A | A | C | D | B | B | C | D | D | C |
| 426 | C | D | D | A | B | D | D | D | A | A |
| 427 | A | A | A | A | A | B | B | C | A | A |
| 428 | D | D | D | A | B | D | D | D | A | B |
| 429 | A | A | A | A | A | B | C | C | A | A |
| 430 | | D | D | | C | | D | D | | C |
| 431 | | A | C | | A | | C | C | | A |
| 432 | | A | A | | A | | C | C | | A |
| 433 | | B | D | | B | | C | D | | B |
| 434 | | C | C | | C | | D | D | | A |
| 435 | | A | A | | A | | C | C | | A |
| 436 | A | B | C | A | B | C | C | D | A | B |
| 437 | A | B | C | D | C | B | C | D | C | C |
| 438 | A | C | D | A | C | C | D | D | A | B |
| 439 | D | D | D | D | D | D | D | D | B | C |
| 440 | C | C | D | B | C | D | D | E | B | C |
| 441 | C | D | D | B | D | D | D | D | A | C |
| 442 | A | A | C | D | A | B | B | C | B | A |
| 443 | C | D | D | C | E | C | D | D | A | B |
| 444 | A | B | D | A | C | C | D | D | A | A |
| 445 | A | A | B | A | A | B | B | C | A | A |
| 446 | B | D | D | A | D | D | D | D | A | B |
| 447 | A | A | C | A | A | B | B | C | A | A |
| 448 | A | C | D | A | C | C | D | D | A | B |
| 449 | D | E | E | D | D | D | E | E | B | C |
| 450 | A | A | C | A | A | B | B | C | A | A |
| 451 | A | B | D | A | C | C | D | D | A | B |
| 452 | A | B | C | A | B | C | C | D | A | A |
| 453 | A | A | B | B | A | C | C | C | A | A |
| 454 | A | A | B | A | A | C | C | D | A | A |
| 455 | A | A | B | A | B | C | C | D | A | B |
| 456 | B | C | D | C | D | D | D | D | A | B |
| 457 | A | C | C | A | C | D | D | D | A | B |
| 458 | A | B | C | A | B | C | D | D | A | B |
| 459 | A | A | C | A | A | B | C | D | A | A |
| 460 | A | A | A | A | A | B | B | C | A | A |
| 461 | A | B | C | A | B | B | C | C | A | A |
| 462 | | A | B | | A | | C | D | | A |
| 463 | A | C | D | A | C | C | D | D | A | B |
| 464 | A | C | D | A | B | C | D | D | A | A |
| 465 | C | D | D | B | D | D | D | D | A | B |
| 466 | A | A | C | A | A | B | B | C | A | A |
| 467 | C | D | D | C | E | D | D | D | B | C |
| 468 | | A | B | | A | | C | C | | A |
| 469 | | D | D | | C | | D | D | | B |
| 470 | B | C | D | A | C | B | B | C | A | B |
| 471a | D | D | D | C | D | D | D | D | B | C |
| 472 | C | D | E | C | C | D | D | D | B | B |
| 473 | C | D | D | B | D | D | D | D | A | C |
| 474 | C | D | D | A | D | D | D | D | A | C |
| 475 | B | C | D | A | C | D | D | D | A | B |
| 476 | D | D | D | D | D | D | D | D | B | C |
| 477 | B | C | D | A | C | C | D | D | A | B |
| 478a | E | E | E | D | E | D | E | E | C | D |
| 479 | D | D | D | C | E | D | D | D | B | C |
| 480 | D | D | D | D | E | D | D | D | C | D |
| 481a | C | D | E | C | D | D | D | D | A | B |
| 482 | D | D | E | D | D | D | D | D | B | C |
| 483 | D | D | E | D | D | D | D | D | B | C |
| 484 | C | D | D | A | D | C | D | D | A | C |
| 485 | C | D | D | C | E | C | D | D | A | C |
| 486a | | A | A | | A | | B | B | | A |
| 486b | | A | A | | A | | B | B | | A |
| 487 | | A | A | | A | | C | C | | A |
| 488 | | A | B | | A | | C | C | | A |
| 490 | | A | A | | A | | D | D | | A |
| 491 | | A | A | | A | | D | D | | A |
| 492 | | D | D | | D | | D | D | | C |
| 493 | | D | E | | E | | D | D | | D |
| 494 | | D | D | | D | | D | D | | C |
| 495 | | C | D | | E | | D | D | | C |
| 496 | | A | A | | A | | B | B | | A |
| 497 | | A | A | | A | | C | C | | A |
| 498 | | A | A | | A | | C | C | | A |
| 499 | | A | A | | A | | C | C | | A |
| 500 | | A | A | | A | | C | C | | A |
| 502 | | A | B | | A | | D | D | | A |
| 503 | | A | C | | C | | C | C | | A |
| 504 | | B | C | | D | | C | D | | B |
| 505 | | C | D | | D | | D | D | | C |
| 506 | | C | D | | C | | C | D | | B |
| 507 | | A | A | | A | | C | C | | A |
| 508 | | A | A | | A | | C | C | | A |
| 509 | | A | A | | A | | C | C | | A |
| 511 | | A | B | | C | | C | C | | B |
| 512 | | B | C | | D | | D | D | | B |
| 513 | | C | D | | B | | D | D | | A |
| 514 | | D | D | | D | | D | D | | B |
| 515 | | A | A | | A | | A | A | | A |
| 516 | | A | A | | A | | A | A | | A |
| 517a | | A | C | | C | | D | D | | B |
| 517b | | A | A | | A | | D | D | | A |
| 519 | | A | A | | A | | C | C | | A |
| 520 | | A | A | | A | | C | C | | A |
| 522 | | D | D | | C | | D | D | | C |
| 523 | | D | D | | D | | D | D | | C |
| 526 | | A | B | | A | | D | D | | A |
| 527 | | B | C | | A | | D | D | | A |
| 528 | | A | A | | A | | D | D | | A |
| 529 | | A | C | | C | | D | D | | B |
| 531 | | A | A | | A | | D | D | | A |
| 532 | | C | D | | C | | D | D | | B |
| 534a | | C | D | | D | | D | D | | C |
| 534b | | C | C | | C | | D | D | | B |
| 535 | | A | A | | A | | C | C | | A |
| 536 | | B | C | | C | | C | C | | A |
| 538 | | C | D | | C | | C | D | | B |
| 540 | | C | D | | B | | D | D | | A |
| 541a | | A | A | | A | | C | C | | A |
| 541b | | A | A | | A | | C | C | | A |
| 543a | | A | C | | A | | B | C | | A |
| 543b | | A | B | | A | | B | B | | A |
| 544 | | D | D | | E | | D | D | | C |
| 545 | | A | A | | A | | B | B | | A |
| 546 | | A | A | | A | | B | B | | A |
| 547 | | D | D | | C | | D | D | | B |
| 548 | | C | D | | C | | D | D | | C |
| 549 | | C | D | | B | | C | D | | B |
| 553a | | A | A | | A | | A | A | | A |
| 553b | | A | A | | A | | A | A | | A |
| 554 | | A | A | | B | | C | C | | A |
| 555 | | A | A | | A | | B | B | | A |
| 556 | | A | A | | A | | A | A | | A |
| 557 | | A | B | | A | | A | A | | A |
| 558a | | A | A | | A | | D | D | | A |
| 558b | | A | A | | A | | D | D | | A |
| 559 | | C | D | | E | | D | D | | C |
| 560 | | B | C | | E | | D | D | | C |
| 561 | | C | D | | E | | D | D | | C |
| 562 | | A | B | | A | | B | B | | A |
| 563 | | C | D | | D | | C | D | | B |
| 564 | | C | D | | C | | C | D | | B |
| 565 | | C | D | | C | | C | C | | A |
| 566 | | A | B | | C | | B | C | | A |
| 567 | | C | D | | D | | D | D | | B |
| 568 | | D | D | | C | | D | D | | B |
| 569 | | A | A | | A | | C | C | | A |
| 570 | | A | C | | A | | B | C | | A |
| 571 | | A | A | | A | | A | A | | A |
| 572 | | A | A | | A | | C | C | | A |
| 573 | | A | B | | A | | B | C | | A |
| 574 | | A | A | | A | | B | B | | A |
| 575 | | A | A | | A | | C | C | | A |
| 576 | | A | A | | A | | A | A | | A |
| 577 | | D | D | | D | | D | D | | C |
| 578 | | B | C | | D | | D | D | | B |
| 579 | | C | D | | E | | D | D | | D |
| 581 | | C | D | | D | | C | D | | B |
| 582 | | A | A | | C | | C | C | | A |
| 583 | | B | C | | B | | B | C | | A |
| 584 | | C | D | | E | | D | D | | C |
| 585 | | B | C | | D | | D | D | | B |
| 586 | | C | D | | E | | D | D | | C |
| 587 | | A | B | | A | | A | A | | C |
| 588 | | A | B | | D | | C | D | | A |
| 589 | | A | A | | A | | A | A | | A |
| 590 | | A | A | | A | | A | A | | A |
| 591 | | A | A | | A | | A | A | | A |
| 592a | | A | B | | A | | D | D | | A |
| 593 | | A | A | | A | | C | C | | A |
| 594 | | B | C | | D | | D | D | | B |
| 595 | | B | C | | D | | C | D | | B |
| 596 | | D | D | | E | | D | D | | C |
| 598 | | D | D | | E | | D | D | | C |
| 599 | | D | E | | E | | D | D | | C |
| 600 | | A | C | | D | | D | D | | A |
| 601 | | C | D | | E | | D | D | | C |
| 602 | | A | B | | C | | C | D | | A |
| 603 | | C | D | | E | | D | D | | C |
| 604 | | C | D | | E | | D | D | | C |
| 605 | | C | D | | E | | D | D | | C |
| 606 | | C | C | | D | | D | D | | B |
| 607 | | C | D | | C | | D | D | | C |
| 608 | | C | D | | D | | D | D | | C |
| 609 | | B | C | | C | | D | D | | B |
| 610 | | A | C | | C | | D | D | | B |
| 611 | | C | C | | C | | C | D | | B |
| 612 | | C | C | | C | | C | D | | B |
| 613 | | B | C | | C | | D | D | | B |
| 614 | | C | C | | C | | D | D | | B |
| 618 | | A | B | | C | | C | D | | A |
| 619 | | B | C | | D | | D | D | | B |
| 620 | | A | A | | A | | C | C | | A |
| 621 | | A | A | | A | | C | C | | A |
| 623 | | A | A | | A | | C | C | | A |
| 624 | | A | B | | A | | C | C | | A |
| 625 | | A | C | | A | | C | C | | A |
| 627 | | A | B | | A | | C | C | | A |
| 628 | | A | B | | A | | C | C | | A |
| 629 | | A | A | | A | | B | B | | A |
| 631 | | A | A | | A | | B | C | | A |
| 632 | | A | A | | A | | B | C | | A |
| 633 | | A | A | | A | | C | C | | A |
| 634 | | | | | | | | | | |
| 635 | | A | A | | A | | C | C | | A |
| 636 | | A | C | | A | | C | C | | A |
| 637 | | A | A | | A | | B | C | | A |
| 641 | | A | A | | A | | B | B | | A |
| 642 | | A | A | | B | | C | C | | A |
| 643 | | A | B | | B | | C | C | | A |
| 644a | | A | C | | C | | C | C | | B |
| 644b | | A | A | | B | | C | C | | A |
| 646 | | A | A | | A | | C | C | | A |
| 649 | | A | B | | A | | C | D | | A |
| 650 | | A | B | | A | | C | D | | A |
| 651 | | A | B | | A | | D | D | | A |
| 652 | | A | B | | A | | C | D | | A |
| 653 | | A | C | | A | | C | C | | A |
| 654 | | A | B | | A | | C | C | | A |
| 655 | | A | B | | A | | C | C | | A |
| 656 | | A | A | | A | | C | C | | A |
| 660 | | A | C | | A | | C | C | | A |
| 662a | | A | B | | A | | D | D | | B |
| 663a | | A | B | | A | | C | C | | A |
| 663b | | A | A | | A | | C | C | | A |
| 668a | | B | C | | C | | B | C | | A |
| 668b | | A | B | | B | | B | C | | A |
| 669a | | B | C | | B | | D | D | | A |
| 669b | | A | C | | A | | C | D | | A |
| 670a | | A | B | | A | | B | B | | A |
| 670b | | A | A | | A | | A | A | | A |
| 671a | | B | C | | A | | A | B | | C |
| 671b | | A | A | | A | | A | A | | A |
| 672a | | A | C | | A | | A | A | | B |
| 672b | | A | A | | A | | A | A | | A |
| 673a | | A | B | | A | | A | A | | B |
| 673b | | A | A | | A | | C | C | | A |
| 674a | | A | A | | C | | B | C | | A |
| 674b | | A | A | | A | | D | D | | A |
| 675a | | B | C | | C | | C | D | | A |
| 675b | | A | A | | A | | C | C | | A |
| 676 | | B | C | | C | | C | D | | B |
| 677 | | D | D | | D | | D | D | | C |
| 679 | | B | C | | C | | D | D | | A |
| 680 | | C | D | | C | | D | D | | B |
| 681 | | D | D | | D | | D | D | | D |
| 682 | | D | D | | D | | D | D | | D |
| 683 | | D | D | | D | | D | D | | D |
| 684 | | E | E | | D | | E | E | | D |
| 686 | | D | D | | E | | D | D | | D |
| 687 | | D | D | | E | | D | D | | D |
| 689a | | D | D | | E | | D | D | | C |
| 689b | | D | D | | E | | D | D | | D |
| 690 | | B | C | | D | | D | D | | B |
| 691a | | A | A | | A | | B | C | | A |
| 691b | | A | C | | B | | B | C | | A |
| 692 | | C | D | | D | | C | D | | B |
| 693 | | A | A | | A | | B | B | | A |
| 694 | | A | A | | D | | C | C | | A |
| 695 | | A | A | | A | | C | C | | A |
| 696 | | D | D | | E | | D | D | | C |
| 697 | | A | B | | A | | C | C | | A |
| 698 | | A | C | | A | | C | D | | A |
| 699 | | C | D | | D | | D | D | | B |
| 700 | | C | D | | E | | C | D | | C |
| 702 | | A | B | | A | | B | B | | A |
| 703 | | A | A | | A | | D | D | | A |

Blank cell in Table indicates data not available.

Conclusions

Cbl-b inhibitors as described herein enhanced IL-2 secretion in T-cells stimulated with anti-CD3 antibody alone or in combination with anti-CD28 antibody. Expression of the surface activation marker, CD25, increased in T-cells stimulated with anti-CD3 antibody alone or in combination with anti-CD28 antibody. These results indicate the identified Cbl-b inhibitors have the ability to activate T-cells and that such activation did not require co-stimulation with anti-CD28 antibody.

### Biological Example 3: Evaluation of Immunomodulatory Effects by Cbl-b Inhibitors

Cbl-b inhibitors identified from screening assays demonstrated the ability to activate total human T-cells in vitro as evidenced by enhanced IL-2 secretion and expression of the CD25 surface activation marker.

Further in vitro studies were conducted to assess additional cytokine secretion by T-cells and expression of surface activation markers on T-cells. Additional immunomodulatory effects on T-cells contacted with the Cbl-b inhibitors described herein were assessed, such as the ability of a Cbl-b inhibitor to increase T-cell proliferation, decrease T-cell exhaustion, and decrease T-cell anergy. The ability of Cbl-b inhibitors, such as those described herein, to activate T-cells in vivo was also assessed. Other immunomodulatory effects by the Cbl-b inhibitors were assessed such as the ability of Cbl-b inhibitors to activate B-cells and NK cells.

Purification and Assessment of Primary Human Total T-Cell Activation

Peripheral blood mononuclear cells (PBMC) were obtained either: 1) by using Ficoll-Paque™ (GE Healthcare) for separation of peripheral blood hematopoietic cells from buffy coats of healthy human donors; or 2) directly from FeukoPak donations. Total human primary T-cells were isolated from the PMBCs utilizing negative selection with commercial kits following the manufacturer's protocol (Miltenyi Catalog #130-096-535 (i.e., cocktail of antibodies against surface markers CD14, CD15, CD16, CD19, CD34, CD36, CD56, CD123 and CD235a are incubated with the PBMCs before passing the samples by magnetic beads for removal of cells expressing those surface markers) or StemCells Catalog #17951) to yield >95% CD3+ cells as assessed by flow cytometry. For measurement of cell proliferation, cells were labeled with Cell Trace Violet (Invitrogen) following manufacturer's protocol prior to activation by stimulation with anti-CD3 antibody alone or in combination with anti-CD28 antibody. The Cbl-b inhibitor was added to 1×10⁵ cells per well at multiple concentrations (e.g., 10 μM, 1.11 μM, or 0.123 μM) with a final DMSO concentration of <0.1%. The plate was incubated for one hour at 37° C. in 5% CO₂. Following incubation with the Cbl-b inhibitor, primary human total T-cells were stimulated with either plate bound anti-CD3 antibody (OKT3) alone or plate bound anti-CD3 antibody (OKT3) with soluble anti-CD28 antibody (28.2) (Life Technologies). To prepare plates with plate bound anti-CD3 antibody (OKT3), 96-well round bottom tissue culture plates were coated with 100 μl of anti-CD3 antibody (OKT3) at 10 μg/mL for 4 hours at 37° C. 5% CO₂ in phosphate buffered saline (PBS). The plates were washed with PBS prior to adding the cells with or without soluble anti-CD28 antibody (28.2) to each well at a final concentration of 5 μg/mL. Cells were stimulated for 48 hours prior to harvesting the cell free supernatant and staining the cell population for surface marker assessment by flow cytometry. Supernatants were analyzed for cytokine secretion (e.g., GM-CSF, IFNγ and TNFα) by ELISA (R&D Systems, Peprotech or Life Technologies) or Luminex multiplex kits (Procarta Life Technologies) following manufacturer's protocol. Cells were stained with anti-CD69 (BD Biosciences) to assess levels of surface markers of activation. Proliferation was measured by flow cytometry and data was analyzed with FlowJo v7.6.5 or v10. Readouts were reported as fold change over baseline. In some embodiments, baseline was the measurement obtained from total human T-cells stimulated with anti-CD3 antibody alone, wherein the cells were not incubated with a Cbl-b inhibitor. In some embodiments, baseline was the measurement obtained from total human T-cells stimulated with anti-CD3 antibody and anti-CD28 antibody, where the cells were not incubated with a Cbl-b inhibitor.

Cbl-b inhibitor effects on primary human T-cells were also evaluated in the context of an allogenic mixed lymphocyte reaction (MLR). Allogenic immature dendritic cells were generated under the following conditions. Peripheral blood mononuclear cells (PBMC) were obtained either: 1) by using Ficoll-Paque™ (GE Healthcare) for separation of peripheral blood hematopoietic cells from huffy coats of healthy human donors; or 2) directly from LeukoPak donations. Monocytes were isolated from the PMBCs utilizing positive selection with a commercial kit following the manufacturer's protocol (StemCells Catalog #17858) to yield >95% CD14+ cells as assessed by flow cytometry. Monocytes were cultured with 30 ng/mL of recombinant human GM-CSF and 20 ng/mL of recombinant human IL-4 for seven days to generate immature dendritic cells. Monocytes and T-cells were either isolated fresh from peripheral blood or thawed from frozen stocks. Human T-cells were isolated, labeled with CFSE and incubated with inhibitors as described above. The Cbl-b inhibitor was added to 1×10⁵ T-cells in coculture with 2×10³ allogenic immature dendritic cells per well at multiple concentrations (e.g., 10 μM, or 1.11 μM) with a final DMSO concentration of <0.1% and incubated at 37° C. in 5% CO₂ for 5 days. Proliferation of the T-cells was evaluated by flow cytometry.

Cbl-b inhibitors were tested to determine their ability to induce or enhance secretion of cytokines from T-cells (e.g., GM-CSF, IFNγ and TNFα) and/or surface expression of cell surface markers on T-cells (e.g., CD69) that is indicative of T-cell activation. Cbl-b inhibitors were also tested to determine their ability to induce or enhance T-cell proliferation. Cbl-b inhibitors were tested for their effects on T-cell activation in the presence of costimulation and where conditions were suboptimal for priming.

Results

Readouts for T-cell activation were reported as fold change over baseline. Baseline for this study was the measurement obtained from total human T-cells stimulated with anti-CD3 antibody alone or anti-CD3 antibody in combination with soluble anti-CD28 antibody, where the cells were not incubated with a Cbl-b inhibitor. For T-cells stimulated with anti-CD3/anti-CD28 or with anti-CD3 alone changes greater than 1,2-fold over baseline for % positive CD69 staining and greater than 2-fold over baseline for % total proliferating cells were considered a significant and positive response (Table 4a). Compounds were ranked into bins according to their readouts for % CD69 positive staining as follows: A indicates ≤1.200, B indicates 1.201-1.400, C indicates 1.401-1.600 and indicates >1.601. Compounds were ranked into bins according to their readouts for % total proliferation as follows: A indicates ≤2.00, B indicates 2.01-3.00, C indicates 3.01-4.00 and indicates >4.00. For T-cells stimulated with anti-CD3/anti-CD28 or anti-CD3 alone, changes greater than 1.5-fold over baseline for GM-CSF and TNFα secretion and greater than 2-fold over baseline for IFNγ secretion was considered a significant and positive response (Table 4b). Compounds were ranked into bins according to their readouts for GM-CSF and TNFα secretion as follows: A indicates ≤1.50, B indicates 1.51-2.00, C indicates 2.01-4.00, and D indicates >4.00. Compounds were ranked into bins according to their readouts for IFNγ secretion as follows: A indicates ≤2.00, B indicates 2.01-3.00, C indicates 3.01-4.00, and D indicates >4.00.

TABLE 4a
T-cell activation assessed by CD69 expression and cell proliferation (CP)

| | CD3/CD28 | | | | | | | | CD3 | | | | | |
|---|---|---|---|---|---|---|---|---|---|---|---|---|---|---|
| | CD4 | | | | CD8 | | | | CD4 | | CD8 | | | |
| Compound | % CD69+ | | % CP | | % CD69+ | | % CP | | % CD69+ | | % CD69+ | | % CP | |
| ID No./μM | 10 | 1.11 | 10 | 1.11 | 10 | 1.11 | 10 | 1.11 | 10 | 1.11 | 10 | 1.11 | 10 | 1.11 |
| 57a | C | B | D | C | B | A | D | B | C | D | B | B | D | C |
| 57b | A | A | A | A | A | A | A | A | A | A | A | A | A | A |
| 140 | D | C | D | D | B | B | D | D | D | D | C | C | D | D |
| 183 | C | B | D | C | B | B | D | B | D | C | C | B | D | C |
| 195 | D | A | D | A | B | A | D | A | D | A | C | A | D | A |
| 202 | B | A | C | A | A | A | B | A | B | A | A | A | A | A |
| 255a | B | A | C | A | B | A | B | A | A | A | A | A | A | A |
| 257 | C | A | D | B | B | A | C | A | B | A | B | A | C | A |
| 282 | B | A | D | A | B | A | B | A | B | A | A | A | C | A |
| 313 | B | C | D | D | B | B | B | C | B | D | A | C | B | D |
| 325 | C | C | D | D | B | B | D | B | D | C | B | A | D | B |
| 326 | C | B | D | B | B | A | C | B | C | B | B | A | D | B |
| 335 | D | C | D | D | B | B | C | C | D | D | C | B | D | D |
| 336 | C | C | D | D | B | B | D | C | D | C | C | B | D | D |
| 369 | C | A | D | A | B | A | B | A | A | A | A | A | A | A |
| 372 | B | A | D | A | B | A | B | A | B | A | B | A | B | A |
| 376 | D | C | D | D | B | B | D | C | D | D | C | B | D | D |
| 385 | C | B | D | D | B | B | C | B | D | C | C | B | D | C |
| 393 | B | A | C | A | A | A | B | A | B | B | A | A | A | A |
| 397 | C | C | D | D | B | B | D | C | D | D | C | B | D | D |
| 398 | C | C | D | D | B | B | D | C | D | D | C | B | D | D |
| 399 | D | D | D | D | B | B | D | D | D | D | C | C | D | D |
| 412 | C | C | D | D | B | B | D | D | D | D | C | C | D | D |
| 413 | D | C | D | D | B | B | D | C | D | C | C | B | D | C |
| 415 | C | B | D | D | B | B | D | C | D | D | C | B | D | D |
| 422 | C | B | D | D | B | B | C | B | D | D | C | B | D | C |
| 438 | C | B | D | C | B | A | C | A | D | B | B | A | D | A |
| 450 | C | B | D | D | B | B | D | B | D | C | C | B | D | D |
| 452 | C | B | D | D | B | B | D | C | D | C | C | B | D | C |
| 455 | C | B | D | D | B | B | D | C | D | C | C | B | D | D |
| 472 | C | B | D | C | B | A | B | B | C | C | B | A | A | B |
| 520 | D | D | D | D | B | B | D | D | D | D | C | C | D | D |

TABLE 4b
T-Cell aCtivation assessed by Cytokine secretion

| | GM-CSF | | | | TNFα | | | | IFNγ | | | |
|---|---|---|---|---|---|---|---|---|---|---|---|---|
| Compound | CD3/CD28 | | CD3 | | CD3/CD28 | | CD3 | | CD3/CD28 | | CD3 | |
| ID No./μM | 10 | 1.11 | 10 | 1.11 | 10 | 1.11 | 10 | 1.11 | 10 | 1.11 | 10 | 1.11 |
| 57a | C | B | C | B | C | B | B | B | A | A | A | A |
| 140 | D | C | C | C | C | C | C | B | D | B | D | C |
| 183 | D | C | C | B | C | C | B | A | D | B | D | B |
| 195 | D | D | C | C | D | C | B | B | D | B | D | B |
| 202 | B | A | B | A | B | A | A | A | D | A | A | A |
| 255a | B | A | A | A | B | A | A | A | D | C | A | A |
| 257 | D | C | C | B | C | B | A | A | B | A | B | A |
| 282 | C | B | C | A | C | A | A | A | B | A | B | A |
| 313 | C | A | B | A | C | A | A | A | B | A | A | B |
| 325 | C | B | B | A | C | A | A | A | D | C | A | A |
| 326 | C | B | C | B | C | A | B | A | B | A | B | A |
| 335 | D | B | C | A | C | B | B | B | B | D | B | A |
| 336 | D | C | C | C | C | C | B | B | D | B | D | B |
| 369 | C | A | A | A | C | A | B | A | A | A | A | A |
| 372 | C | A | B | A | C | A | A | A | A | A | A | A |
| 376 | D | C | C | B | C | C | B | A | A | A | C | B |
| 385 | C | B | C | B | C | B | B | A | D | A | A | A |
| 393 | B | A | A | A | B | A | A | A | D | B | A | A |
| 397 | D | C | C | C | C | C | B | B | D | B | A | A |
| 398 | D | C | D | C | C | C | C | A | D | C | A | A |
| 399 | D | D | D | C | C | C | B | C | D | D | D | C |
| 412 | D | D | D | D | D | C | C | B | D | D | D | C |
| 413 | D | C | C | B | C | C | B | B | C | A | D | B |
| 415 | D | C | D | C | C | C | B | B | D | C | D | B |
| 422 | D | C | C | C | C | C | B | A | D | C | C | A |
| 438 | C | B | C | A | C | A | B | C | B | A | B | A |
| 450 | D | C | C | B | C | C | B | B | D | B | A | A |
| 452 | C | C | C | B | C | B | B | A | B | A | C | C |
| 455 | D | C | C | B | C | C | C | A | D | D | C | B |
| 472 | C | B | B | B | B | B | A | A | A | A | A | A |
| 520 | D | D | D | C | D | C | C | C | D | D | D | D |

Readouts for the allogenic mixed lymphocyte reaction were reported as fold change over baseline. Baseline for this study was the measurement obtained from total human T-cells stimulated with allogenic immature dendritic cells, wherein the T-cells were not incubated with a Cbl-b inhibitor (Table 5). Increases in proliferation greater than 2-fold over baseline were considered a positive response (Table 5). Compounds were ranked into bins according to their readouts as follows: A indicates ≤2.00, B indicates 2.01-3.00, and C indicates >3.01.

TABLE 5
Allogenic T-cell activation assessed by cell proliferation

Proliferation

| Compound ID No. | 10 μM | 3 μM |
|---|---|---|
| 57a | A | A |
| 57b | A | A |
| 140 | A | A |
| 183 | A | A |
| 195 | B | A |
| 202 | A | A |
| 255a | A | A |
| 257 | A | A |
| 282 | A | A |
| 302 | A | A |
| 313 | A | A |
| 325 | A | A |
| 326 | A | A |
| 335 | A | A |
| 336 | C | A |
| 369 | A | A |
| 372 | A | A |
| 376 | A | A |
| 385 | A | A |
| 393 | B | A |
| 397 | B | A |
| 398 | B | B |
| 399 | C | B |
| 412 | C | C |
| 413 | C | B |
| 415 | C | B |
| 422 | B | A |
| 438 | A | A |
| 442 | A | A |
| 450 | B | A |
| 452 | A | A |
| 455 | A | A |
| 472 | A | A |
| 519 | C | C |
| 520 | C | C |

Conclusions

Cbl-b inhibitors identified from screening assays enhanced T-cell activation measured by an increase in CD69 expression, an early surface marker of activation, and secretion of inflammatory cytokines. Cbl-b inhibitors also increased the proliferation of primary human T-cells in response to anti-CD3/anti-CD28 activation and in an allogenic MLR assay. These results indicate the identified Cbl-b inhibitors have the ability to enhance the activity and proliferation of T-cells in the presence of costimulation and under suboptimal priming conditions.

Human T-Cell In Vitro Models of T-Cell Exhaustion

T-cell exhaustion is characterized by cells having a poor effector response and a sustained level of inhibitory receptor expression that results in T-cell dysfunction in response to chronic infections and cancer. In vitro models of T-cell exhaustion include allogenic and autologous models. In an autologous model, myeloid cells and SEB (Staphylococcal enterotoxin B, Millipore) were used to stimulate anti-CD3 stimulated T-cells. Peripheral blood mononuclear cells (PBMC) were obtained either: 1) by using Ficoll-Paque™ (GE Healthcare) for separation of peripheral blood hematopoietic cells from huffy coats of healthy human donors; or 2) directly from LeukoPak donations. Monocytes were isolated with commercial kits using negative selection with StemCells Easy Sep Human Monocyte Enrichment Kit without CD16 Depletion (Catalog #19058) following the manufacturer's protocol. Isolated monocytes were cultured in complete media (e.g. RPMI 1640 with no additives, 10% HI FBS, IX Glutamine and IX p-mercaptoethanol) with 50 ng/mL recombinant human M-CSF (R&D System or Peprotech). Cells were plated at 2×10⁶ cells per well (Day 0) and cultured for 5 days and were fed with fresh media and cytokines on day 2. On day 5 IFNγ was added at 100 ng/mL and the cells were incubated overnight. Primary human T-cells from the same donor were isolated from PBMCs with a commercial kit using negative selection (with StemCell Easy Sep Human T-cell Isolation Kit (Catalog #17951) following the manufacturer's protocol. Purity was confirmed by surface marker detection by flow cytometry for CD4, CD8, CD45RA, CD45RO, CD19, CD14, CD56, and CD3 (BD Biosciences). 3×10⁶ cells per/mL T-cells were stimulated with 10 μg/mL of plate bound anti-CD3 antibody (Clone UCHT-1) for 5 days. This was done in parallel with myeloid cell generation. On day 6, 2.5×10⁴ T-cells were added per well, 12.5×10³ myeloid cells per well and SEB antigen (0.1 μg/mL) were added to wells of a round bottom 96-well plate. Test agents (e.g. Cbl-b inhibitor compounds) or controls (e.g., checkpoint neutralizing antibodies such as anti-PD1 antibody) were added to the wells at the indicated concentrations (e.g. 10 μM). Cells were cultured for 3 days at which point cell free supernatants were collected and assessed for secreted cytokines (e.g., GM-CSF, IFNγ, and IL-2) by ELISA (R&D Systems, Peprotech or Life Technologies) or Luminex multiplex kits (Procarta Life Technologies). The T-cells were stained for a panel of surface markers including checkpoint inhibitors (e.g. CTLA4) and evaluated by flow cytometry for Cbl-b inhibitor effects.

Cbl-b inhibitors were tested to determine their ability to induce or enhance secretion of cytokines from exhausted T-cells (e.g., GM-CSF, IFNγ and IL-2) in the presence of myeloid cells, which is indicative of decreased T-cell exhaustion. Cbl-b inhibitors were also tested for their effects on checkpoint modulator expression levels following activation of exhausted T-cells.

Results

Readouts were reported as fold change over baseline. Baseline for this study was the measurement obtained from in vitro generated exhausted human T-cells stimulated with SEB in the presence of autologous myeloid cells, in which the T-cells were not incubated with a Cbl-b inhibitor (Table 6). Changes greater than 1,4-fold over baseline for IFNγ, GM-CSF and CTLA4, and greater than 2.0-fold over baseline for IL-2, were considered a positive response (Table 6). Compounds were ranked into bins according to their readouts as follows for IFNγ, GM-CSF and CTLA4: A indicates ≤1.40, B indicates 1.41-1.60 and C indicates >1.61. Compounds were ranked into bins according to their readouts as follows for IL2: A indicates ≤2.50, B indicates 2.51-3.00 and C indicates >3.01.

TABLE 6
Exhausted T-cell activation assessed by
cytokine secretion and CTLA4 expression

| | | |
|---|---|---|
| Compound | Cytokines | CTLA4 |

| ID No. | IFNγ | IL-2 | GM-CSF | CD4 | CD8 |
|---|---|---|---|---|---|
| 57a | A | A | B | A | A |
| 57b | A | A | A | A | A |
| 140 | A | A | A | A | A |
| 183 | B | A | C | C | C |
| 195 | B | A | C | C | C |
| 202 | A | A | A | A | A |
| 255a | A | A | A | B | B |
| 257 | C | A | A | A | A |
| 282 | B | B | A | A | A |
| 302 | A | A | A | A | A |
| 313 | A | A | A | A | A |
| 325 | A | A | A | A | A |
| 326 | C | C | A | A | A |
| 335 | B | C | A | C | C |
| 336 | B | C | C | A | A |
| 369 | A | A | A | A | A |
| 372 | A | A | A | A | A |
| 376 | A | B | A | C | C |
| 385 | B | A | A | C | C |
| 393 | C | C | B | C | C |
| 397 | C | B | B | C | C |
| 398 | C | B | C | B | C |
| 399 | C | C | C | C | C |
| 412 | C | A | B | A | A |
| 413 | C | C | C | C | C |
| 415 | C | A | C | C | C |
| 422 | A | A | B | C | C |
| 438 | A | B | A | A | A |
| 442 | B | C | A | | |
| 450 | C | C | B | C | C |
| 452 | C | B | C | C | C |
| 455 | C | A | C | C | C |
| 472 | C | A | A | B | B |
| 519 | C | B | C | C | C |
| 520 | A | B | B | C | C |

Conclusions

Cbl-b inhibitors identified from screening assays increased exhausted T-cell activation measured by an increase in secreted cytokines. Cbl-b inhibitors also increased checkpoint modulators on exhausted primary human T-cells. These results indicate the identified Cbl-b inhibitors have the ability to enhance the cytokine response and checkpoint modulation of in vitro generated exhausted T-cells.

Human T-Cell In Vitro Models of T-Cell Anergy

Peripheral blood mononuclear cells (PBMC) were obtained either: 1) by using Ficoll-Paque™ (GE Healthcare) for separation of peripheral blood hematopoietic cells from buffy coats of healthy human donors; or 2) directly from LeukoPak donations. Total human primary T-cells were isolated from the PBMCs utilizing negative selection with commercial kits following the manufacturer's protocol (Miltenyi Catalog #130-096-535 (i.e., cocktail of antibodies against surface markers CD14, CD15, CD16, CD19, CD34, CD36, CD56, CD123 and CD235a were incubated with the PBMCs before passing the samples by magnetic beads for removal of cells expressing those surface markers) or StemCells Catalog #17951) to yield >95% CD3+ cells assessed by flow cytometry. The cells were activated with immobilized anti-CD3 antibody (OKT3) and soluble anti-CD28 antibody (28.2) for two days at which time they are washed and allowed to rest for three days in the absence of stimulation. They were then treated with ionomycin (Sigma) for 18-24 hours to induce anergy. Following two washes to remove the ionomycin from the samples, Cbl-b inhibitor compounds were added to the cells at the indicated concentrations (e.g. 10, 1.11 and 0.37 μM) and incubated for 1 hour. The cells were then re-challenged with anti-CD3 antibody and anti-CD28 antibody for 24 hours at which point cell free supernatants were collected and assessed for cytokines (e.g., IFNγ) by ELISA (R&D Systems or Peprotech) or Luminex multiplex kits (Procarta Life Technologies) following manufacturer's protocols.

Cbl-b inhibitors were tested to determine their ability to induce or enhance secretion of cytokines from anergic T-cells (e.g. IFNγ), which is indicative of decreased T-cell tolerance.

Results

Readouts were reported as fold change over baseline. Baseline for this study was the measurement obtained from anergic human T-cells stimulated with immobilized anti-CD3 and soluble anti-CD28, wherein the T-cells were not incubated with a Cbl-b inhibitor (Table 7). For IFNγ changes greater than 2.5-fold over baseline were considered a positive response (Table 7). Compounds were ranked into bins according to their readout as follows for IFNγ: A indicates ≤2.50, B indicates 2.51-4.00 and C indicates >4.01.

TABLE 7
Anergic T-cell activation assessed by cytokine secretion

| | IFNγ Fold change from baseline | | |
|---|---|---|---|
| Compound ID No. | 10 μM | 1.11 μM | 0.37 μM |
| 57a | B | B | A |
| 57b | A | A | A |
| 140 | B | A | A |
| 183 | A | B | A |
| 195 | A | A | A |
| 202 | B | A | A |
| 255a | A | B | A |
| 257 | B | A | A |
| 282 | A | A | A |
| 302 | A | A | A |
| 313 | A | A | A |
| 325 | A | A | A |
| 326 | B | A | A |
| 335 | C | B | A |
| 336 | C | B | B |
| 369 | B | A | A |
| 372 | B | A | A |
| 376 | C | A | A |
| 385 | C | A | A |
| 393 | C | B | A |
| 397 | C | C | A |
| 398 | C | C | A |
| 399 | C | C | C |
| 412 | C | C | A |
| 413 | C | C | B |
| 415 | C | B | B |
| 422 | B | B | A |
| 438 | C | A | A |
| 442 | B | B | A |
| 450 | C | B | B |
| 452 | C | B | A |
| 455 | C | B | A |
| 472 | B | A | A |
| 519 | C | C | C |
| 520 | C | B | A |

Conclusions

Cbl-b inhibitors identified from screening assays increased anergic T-cell activation measured by an increase in IFNγ secretion. These results indicate the identified Cbl-b inhibitors have the ability to modulate tolerized T-cell responses.

In Vivo Activity of Cbl-b Inhibitors

A method of determining the pharmacodynamic profile of Cbl-b inhibitors was performed by dosing strains of mice with competent immune systems such as C57BL/6 or BALB/c with a Cbl-b inhibitor. The Cbl-b inhibitor was dissolved in a suitable formulation and administered by one of various routes such as intravenous (IV), intraperitoneal (IP), subcutaneous (SC), or oral (PO), at a suitable dose level and frequency (e.g. twice per day BID or thrice per day TID) as informed by prior pharmacokinetic and tolerability studies. Following administration of the Cbl-b inhibitor, T-cells and indirectly other immune cells (e.g. via cytokine production) were stimulated in vivo by administration of an anti-CD3 antibody or antigen-binding fragment thereof in PBS at defined amounts such as 2 μg or 10 μg per animal by routes such as IV or IP (See Hirsh et al., J. Immunol., 1989; Ferran, et al., Eur. J. Immunol., 1990). Additional study control arms included groups of mice treated with a vehicle formulation alone (i.e., formulation without the Cbl-b inhibitor and anti-CD3 antibody), a formulation containing the Cbl-b inhibitor alone, a formulation containing the anti-CD3 antibody alone, PBS alone, or combinations of these agents. The level of immune activation was then assessed by analysis of plasma cytokine levels and/or expression of activation markers on immune cells (e.g. T-cells). Blood or lymphoid organs (e.g. spleen) were collected at defined time points (e.g. 8 hours or 24 hours). Blood samples were processed to collect plasma for determination of cytokine levels using standard methods known in the art. Cytokines measured included IL-2, IFNγ, and TNFα. Additional blood samples and lymphoid tissues were processed for flow cytometric analysis of immune cells (e.g. T-cells) using standard methods to determine expression of cell type-specific markers and activation markers such as CD25 and/or CD69. Augmentation of immune stimulation by Cbl-b inhibitor administration was assessed by comparing the relative concentrations of cytokines in plasma, or the expression levels of activation markers on immune cells between appropriate groups (e.g. mice treated with Cbl-b inhibitor and 2 μg anti-CD3 antibody versus mice treated with vehicle and 2 μg anti-CD3 antibody).

Cbl-b inhibitors were tested to determine their ability to induce or enhance the level of cytokines (e.g. IL-2, IFNγ, and TNFα) in blood obtained from treated mice stimulated with an anti-CD3 antibody, which is indicative of modulation of the immune response. Cbl-b inhibitors were also tested to determine their ability to induce or enhance the expression of cell surface markers on T-cells (e.g. CD25 and/or CD69) isolated from treated mice stimulated with an anti-CD3 antibody, which is indicative of modulation of the immune response.

Results

Readouts were reported as fold change over baseline. The fold change was calculated from the mean value of mice treated with Cbl-b inhibitor and 2 μg anti-CD3 antibody, with the baseline defined as the mean value of mice treated with vehicle and 2 μg anti-CD3 antibody. Increases in the percentage of cells with surface expression of CD25 on CD4+ T-cells or CD8+ T-cells greater than 1.5-fold over baseline at 24 hours were considered a positive response. Increases in the plasma concentration of IL-2, IFNγ, and TNFα greater than 1.5-fold over baseline at 8 hours post-stimulation were also considered a positive response (Table 8). Fold changes were ranked into bins according to their readouts as follows: A indicates >2.50, B indicates 2.01-2.50, C indicates 1.51-2.00, D indicates 1.00-1.50, and E indicates <1.00.

TABLE 8
In vivo activity of Cbl-b inhibitors at various doses as assessed by
CD25 expression on T-cells and cytokine levels in plasma\hat{ }

| | | CD4+ T-cell % CD25+ | | | | CD8+ T-cell % CD25+ | | | | IL-2 | IFNγ | TNFα |
|---|---|---|---|---|---|---|---|---|---|---|---|---|
| Compound | Route & | 45 | 90 | 135 | 180 | 45 | 90 | 135 | 180 | 90 | 90 | 90 |
| ID No. | Schedule | mpk | mpk | mpk | mpk | mpk | mpk | mpk | mpk | mpk | mpk | mpk |
| 57a | IP BID | | D | | | | E | | | A | D | A |
| 57a | IP TID | | D | | | | C | | | A | B | A |
| 57b | IP BID | | C | | | | E | | | E | E | D |
| 57b | IP TID | | E | | | | D | | | E | E | E |
| 183 | IP BID | | D | | | | D | | | | | |
| 187 | IP BID | | C | | | | D | | | | | |
| 191 | IP BID | C | | | | D | | | | | | |
| 191 | PO BID | | D | D | B | | D | D | D | | | |
| 195 | PO BID | | D | D | C | | D | D | D | | | |
| 255a | IP BID | | B | | | | D | | | | | |
| 255a | PO BID | | | D | | | | E | | | | |
| 255b | PO BID | | | E | E | | | E | E | | | |
| 282 | PO BID | | D | C | B | | D | D | D | | | |
| 336 | PO BID | D | D | C | C | D | D | D | D | | | |
| 386 | PO BID | | | C | | | | D | | | | |
| 410 | PO BID | | D | D | D | | D | D | D | | | |
| 412 | PO BID | | D | C | B | | D | D | D | | | |
| 413 | PO BID | | D | D | D | | D | D | D | | | |
| 415 | PO BID | | D | D | D | | D | E | E | | | |
| 421 | PO BID | | E | E | E | | E | E | E | | | |
| 429 | PO BID | | | D | | | | D | | | | |
| 432 | PO BID | | | D | | | | D | | | | |
| 447 | PO BID | | D | D | C | | E | E | D | | | |
| 448 | PO BID | | D | D | D | | E | E | E | | | |
| 487 | PO BID | | | C | | | | D | | | | |
| 489 | PO BID | | | C | | | | D | | | | |
| 496 | PO BID | | | C | | | | D | | | | |
| 519 | PO BID | C | A | | A | D | D | | C | | | |
| 520 | PO BID | D | D | C | B | D | D | D | D | | | |
| 541a | PO BID | | | D | | | | D | | | | |
| 553a | PO BID | | | D | | | | D | | | | |
| 553b | PO BID | | | A | | | | D | | | | |
| 575 | PO BID | C | A | | A | D | C | | C | | | |
| 620 | PO BID | | D | D | D | | D | E | E | | | |
| 622a | PO BID | | | B | | | | D | | | | |
| 622b | PO BID | | | B | | | | D | | | | |
| 663b | PO BID | D | | C | | D | | D | | | | |
| 670a | PO BID | | | B | | | | D | | | | |
| 670b | PO BID | | | C | | | | D | | | | |

\hat{ }Cbl-b inhibitor dose shown as milligrams per kilogram (mpk).

Conclusions

Cbl-b inhibitors identified from screening assays increased in vivo T-cell activation as measured by an increase in the percentage of surface CD25 expression on CD4+ or CD8+ T-cells and an increase in plasma cytokine levels from mice treated with Cbl-b inhibitors and an anti-CD3 antibody. These results indicate that the identified Cbl-b inhibitors have the ability to enhance T-cell activation and plasma cytokine levels in vivo.

B Cell Activation Assay

Peripheral blood mononuclear cells (PBMC) were obtained either: 1) by using Ficoll-Paque™ (GE Healthcare) for separation of peripheral blood hematopoietic cells from buffy coats of healthy human donors; or 2) directly from LeukoPak donations. Human primary B cells were isolated from the PBMCs utilizing negative selection with commercial kits following the manufacturer's protocol (StemCells Catalog #17954) to yield >95% CD20+ cells assessed by flow cytometry. Primary human B cells were plated at 0.7-1×10⁵ per well in a 96-well plate with Cbl-b inhibitors over a dose ranging from 10 μM to 1 nM and incubated at 37° C. 5% CO₂, with a final DMSO concentration of <0.5%. Cells were stimulated with anti-IgM for 20 hours at 37° C. 5% CO₂. Surface activation markers on mature CD20⁺ IgD⁺ B cells were monitored by FACS using an anti-CD69 antibody (BD Biosciences).

Cbl-b inhibitors were tested to determine their ability to induce or enhance surface expression of cell surface markers on B cells (e.g., CD69), which is indicative of B cell activation.

Results

Readouts were reported as area under curve (AUC) of the dose response curve of CD69 mean fluorescence intensity (MFI) normalized to the DMSO control. Baseline for this study was the measurement obtained from primary human B cells treated with DMSO in the presence of 2.5 μg/mL of anti-IgM-F(ab′)₂. For B cells treated with Cbl-b inhibitors and stimulated with anti-IgM, AUC greater than 0.5 were considered a positive response (Table 9). Compounds were ranked into bins according to their readouts as follows: A indicates ≤0.5, B indicates 0.5-1.00, C indicates 1.01-1.50, and D indicates ≥1.51.

TABLE 9
B cell activation assessed by surface marker expression

| Compound ID No. | CD69 MFI AUC |
|---|---|
| 57a | B |
| 57b | A |
| 140 | A |
| 183 | C |
| 195 | D |
| 202 | C |
| 255a | C |
| 257 | B |
| 282 | B |
| 302 | B |
| 313 | B |
| 325 | C |
| 326 | A |
| 335 | B |
| 336 | D |
| 369 | C |
| 372 | A |
| 376 | B |
| 385 | C |
| 393 | B |
| 397 | B |
| 398 | B |
| 399 | C |
| 412 | D |
| 413 | C |
| 415 | C |
| 422 | C |
| 438 | A |
| 442 | B |
| 450 | C |
| 452 | D |
| 455 | C |
| 472 | C |
| 519 | D |
| 520 | D |

Conclusions

Cbl-b inhibitors identified from screening assays enhanced anti-IgM activation of B cells as measured by an increase in CD69, an early surface marker of activation. These results indicate the identified Cbl-b inhibitors have the ability to enhance the activity of B cells.

Purification and Activation of Primary Human NK Cells.

Peripheral blood mononuclear cells (PBMC) were obtained either: 1) by using Ficoll-Paque™ (GE Healthcare) for separation of peripheral blood hematopoietic cells from buffy coats of healthy human donors; or 2) directly from LeukoPak donations. Total human primary NK cells were isolated from the PBMCs utilizing negative selection with commercial kits following the manufacturer's protocol (Miltenyi Catalog #130-092-657 or StemCells Catalog #17955) to yield >92% CD56+, CD3− cells as assessed by flow cytometry. The cells were cultured overnight with IL-2 (60 ng/mL) at 37° C. 5% CO₂. Cbl-b inhibitors were added 1 hour prior to stimulation and incubated at 37° C. 5% CO₂ at a specific concentration (e.g., 10 μM, 1 μM, or 0.1 μM) with a final DMSO concentration of <0.1%. NK cells were co-cultured with target cells that were engineered to have a red nucleus (K562 NucRed) measurable by flow cytometry. K562 NucRed cells were produced by transduction of K562 cells with IncuCyte NucLight Red Lentivirus reagent (Catalog #4476) and selected for 5 days. Clonal populations were isolated and expanded using standard tissue culture techniques, and individual clones were validated by comparison to wildtype K562 cells in NK cell killing assays. The cells were mixed at the indicated ratios (e.g., 5:1, 1:1, or 1:5) of NK (effector cells) to K562 NucRed (target cells) for 6 hours. Cell free supernatants were collected and analyzed for cytokine secretion (e.g. TNFα, IFNγ, or MIP1β) by ELISA or Luminex multiplex kits following the manufacturer's protocol. IFNγ secretion was assessed using an R&D Systems ELISA kit (Catalog #DY285), TNFα secretion was assessed using an R&D Systems ELISA kit (Catalog #DY210), and MIP1β secretion was assessed using an R&D Systems ELISA kit (Catalog #DY271).

Results

Readouts were reported as fold change over baseline. Baseline for this study was the measurement obtained from primary human NK effector cells cocultured with K562 NucRed target cells in the absence of a Cbl-b inhibitor. For NK cells cocultured with K562 NucRed cells, changes greater than 2.0-fold over baseline for cytokine secretion were considered a positive response (Table 10a and 10b). Compounds were ranked into bins according to their readouts as follows: A indicates ≤1.00-fold, B indicates 1.01-2.00-fold, C indicates 2.01-5.00-fold, and D indicates ≥5.00-fold.

TABLE 10a
NK cell activation assessed by cytokine secretion\hat{ }

| | IFNγ | | IFNγ | | TNFα | | TNFα | | MIP1β | | MIP1β | |
|---|---|---|---|---|---|---|---|---|---|---|---|---|
| | 5:1 E/T | | 1:1 E/T | | 5:1 E/T | | 1:1 E/T | | 5:1 E/T | | 1:1 E/T | |
| Compound | 10 | 1 | 10 | 1 | 10 | 1 | 10 | 1 | 10 | 1 | 10 | 1 |
| ID No. | μM | μM | μM | μM | μM | μM | μM | μM | μM | μM | μM | μM |
| 57b | B | A | B | A | A | A | B | B | B | A | B | B |
| 57a | C | B | C | B | C | B | B | B | B | A | B | B |
| 9b | A | A | A | A | A | A | A | A | A | A | A | B |
| 255a | D | B | C | B | A | A | A | A | B | A | B | A |
| 220 | C | B | C | B | C | B | C | B | C | B | C | B |
| 191 | D | C | C | C | C | C | B | C | B | B | B | B |
| 195 | D | C | C | C | D | C | C | C | C | C | C | B |
| 187 | D | C | C | B | D | C | C | A | C | B | C | B |
| 447 | D | D | D | C | D | C | C | C | C | B | B | A |
| 448 | D | B | D | B | C | B | C | B | C | B | C | B |
| 296a | B | A | C | B | A | A | B | A | A | A | A | A |
| 438 | B | A | A | A | B | B | B | B | B | A | A | A |
| 399 | D | C | D | C | D | C | C | C | B | B | B | B |

E/T refers to the Effector:Target cell Ratio

TABLE 10b
NK cell activation assessed by cytokine secretion

| | IFNγ | | | TNFα | | |
|---|---|---|---|---|---|---|
| | 1:1 E/T | | | 1:1 E/T | | |
| Compound | 10 | 2.5 | 0.625 | 10 | 2.5 | 0.625 |
| ID No. | μM | μM | μM | μM | μM | μM |
| 57a | C | B | A | C | B | B |
| 57b | B | B | A | C | B | B |
| 140 | B | A | A | A | A | A |
| 183 | C | C | C | C | C | B |
| 195 | D | C | C | C | C | B |
| 202 | C | B | B | B | B | A |
| 255a | C | B | B | C | B | B |
| 257 | C | C | B | C | B | B |
| 282 | C | B | B | C | B | B |
| 302 | C | B | A | B | B | B |
| 313 | C | B | A | B | B | B |
| 325 | C | B | B | B | A | A |
| 326 | C | C | B | C | C | B |
| 335 | C | C | B | C | B | B |
| 336 | D | D | C | C | C | B |
| 369 | C | B | A | B | A | A |
| 372 | C | B | B | B | A | A |
| 376 | C | B | B | C | B | B |
| 385 | C | C | B | B | B | A |
| 393 | C | C | B | C | C | B |
| 397 | D | C | C | C | C | B |
| 398 | D | C | C | C | C | B |
| 399 | D | D | C | D | D | C |
| 412 | D | C | C | C | C | C |
| 413 | D | D | C | C | C | C |
| 415 | D | C | C | C | C | B |
| 422 | C | C | B | C | B | B |
| 438 | C | B | B | B | B | B |
| 442 | C | C | C | B | C | C |
| 450 | D | D | C | C | C | B |
| 452 | D | D | D | C | C | B |
| 455 | D | D | D | C | C | B |
| 472 | C | B | A | B | A | A |
| 519 | D | D | D | C | B | D |
| 520 | D | D | D | C | D | C |

Conclusions

Cbl-b inhibitors obtained from screening assays enhanced cytokine secretion by NK cells activated with K562 NucRed target cells. These results indicate the identified Cbl-b inhibitors have the ability to enhance the activity of NK cells.

The disclosures of all publications, patents, patent applications and published patent applications referred to herein by an identifying citation are hereby incorporated herein by reference in their entirety.

Although aspects of the foregoing invention have been described in some detail by way of illustration and example for purposes of clarity of understanding, it is apparent to those skilled in the art that certain changes and modifications will be practiced. Therefore, the description and examples should not be construed as limiting the scope of the invention.

What is claimed is:

1. A compound of Formula (I-A): 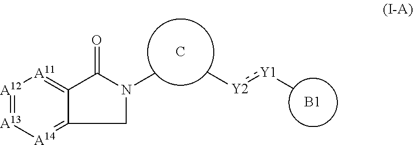
or a tautomer thereof, or a pharmaceutically acceptable salt of any of the foregoing, wherein
A¹¹ is CR¹¹ or N,
A¹² is CR¹² or N,
A¹³ is CR¹³ or N, and
A¹⁴ is CR¹⁴ or N,
wherein no more than two of A¹¹, A¹², A¹³, and A¹⁴ are N;
R¹¹, R¹², R¹³, and R¹⁴ are independently selected from the group consisting of:
H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl
—NR^{j}R^{k} where R^{j} and R^{k} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{k} can be additionally chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{j} and R^{k} are taken together with the nitrogen to which they are attached to form a four- to eight-membered heterocyclic ring or a five-to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a —C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring,
(C₁-C₄ alkylene)-NR^{l}R^{m}, —O—(C₁-C₄ alkylene)-NR^{l}R^{m}, —C(═O)NR^{l}R^{m}, —(C₁-C₄ alkylene)-C(═O)NR^{l}R^{m}, or —O—(C₁-C₄ alkylene)-C(═O)NR^{l}R^{m}, wherein R^{l} and R^{m} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{l} and R^{m} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three-to eight-membered heterocyclic ring where the carbocyclic or heterocyclic ring is optionally substituted with C₁-C₄ alkyl or OH,
—S(═O)₂—C₁-C₈ alkyl,
—SF₅, and
—S(═O)₂NRⁿR^{o} wherein Rⁿ and R^{o} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or Rⁿ and R^{o} are taken together with the nitrogen to which they are attached to form a four- to eight-membered heterocyclic ring or a five-to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
Ring C, 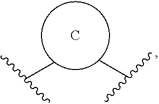
is selected from the group consisting of: 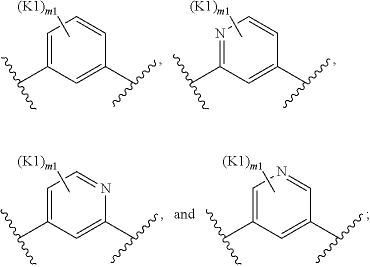
each K1 is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —C₁-C₈ alkyl, —OH or —O—C₁-C₈ alkyl,
—O—C₁-C₈ alkyl optionally substituted with —OH,
—O—C₁-C₈ haloalkyl,
—O-(three- to six-membered heterocyclic ring) optionally substituted with C₁-C₈ alkyl,
a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K1 groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —C₁-C₈ haloalkyl, —O—C₁-C₈ alkyl, and —NR^{g1}R^{h1}, where R^{g1} and R^{h1} are independently H or C₁-C₈ alkyl;
m1 is 0, 1, or 2; [symbol]
is a single bond or a double bond,
wherein when [symbol] is a single bond,
Y1 is C(R¹⁹)(R²⁰), S, or O; and
Y2 is C(R¹⁷)(R¹⁸), and
when [symbol] is a double bond,
Y1 is C(R¹⁹); and
Y2 is C(R¹⁸),
R¹⁷ is selected from the group consisting of H, F, —OH, C₁-C₈ alkyl optionally substituted with —OH or —O—C₁-C₈ alkyl, C₂—C's alkenyl, C₁-C₈ haloalkyl, —O—C₁-C₈ haloalkyl, and —O—C₁-C₈ alkyl optionally substituted with —OH or —O—C₁-C₈ alkyl,
R¹⁸ is selected from the group consisting of H, F, —OH, C₁-C₈ alkyl optionally substituted with —OH, halogen or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl, or
when Y1 is C(R¹⁹)(R²⁰) or C(R¹⁹), R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl, or
R¹⁷ and R¹⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or C₁-C₈ alkylene-OH;
R¹⁹ and R²⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or
R¹⁹ can be taken together with R¹⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl;
or
R¹⁹ and R²⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl;
and
Ring B1, 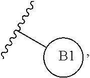
is a five-membered heteroaryl ring containing at least one N, O, or S ring atom, wherein Ring B1 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

2. The compound or tautomer thereof, or pharmaceutically acceptable salt of the compound or tautomer, of claim 1, wherein the compound of Formula (I-A) is a compound of Formula (I): 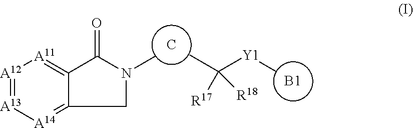
or a tautomer thereof, or a pharmaceutically acceptable salt of any of the foregoing,
wherein
A¹¹ is CR¹¹ or N,
A¹² is CR¹² or N,
A¹³ is CR¹³ or N, and
A¹⁴ is CR¹⁴ or N,
wherein no more than two of A¹¹, A¹², A¹³, and A¹⁴ are N;
R¹¹, R¹², R¹³, and R¹⁴ are independently selected from the group consisting of:
H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{c}, where R^{B} and R^{c} are independently H, C₁-C₈ alkyl, or C₁-C₈ haloalkyl,
—NR^{j}R^{k} where R^{j} and R^{k} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{k} can be additionally chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{j} and R^{k} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring,
(C₁-C₄ alkylene)-NR^{l}R^{m}, —O—(C₁-C₄ alkylene)-NR^{l}R^{m}, —C(═O)NR^{l}R^{m}, —(C₁-C₄ alkylene)-C(═O)NR^{l}R^{m}, or —O—(C₁-C₄ alkylene)-C(═O)NR^{l}R^{m}, wherein R^{l} and R^{m} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{l} and R^{m} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH,
—S(═O)₂—C₁-C₈ alkyl,
—SF₅, and
—S(═O)₂NRⁿR^{o} wherein Rⁿ and R^{o} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or Rⁿ and R^{o} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
Ring C, 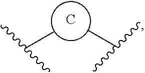
is selected from the group consisting of: 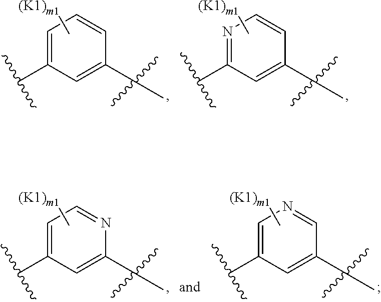
each K1 is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —OH or —O—C₁-C₈ alkyl,
—O—C₁-C₈ alkyl optionally substituted with —OH,
—O—C₁-C₈ haloalkyl,
—O-(three- to six-membered heterocyclic ring) optionally substituted with C₁-C₈ alkyl,
a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, and four- to eight-membered heterocyclyl optionally substituted with —OH or C₁-C₄ alkyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K1 groups are taken together with the atoms to which they are attached to form a three- to six-membered carbocyclic or heterocyclic ring, a phenyl ring, or a five- to six-membered heteroaryl ring, wherein the ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —C₁-C₈ haloalkyl, —O—C₁-C₈ alkyl, and —NR^{g1}R^{h1}, where R^{g1} and R^{h1} are independently H or C₁-C₈ alkyl;
m1 is 0, 1, or 2;
R¹⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,
R¹⁸ is selected from the group consisting of H, F, —OH, C₁-C₈ alkyl optionally substituted with —OH, halogen or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl, or
when Y1 is C(R¹⁹)(R²⁰), R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl, or R¹⁷ and R¹⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or C₁-C₈ alkylene-OH;
Y1 is C(R¹⁹)(R²⁰) or S;
R¹⁹ and R²⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or
R¹⁹ can be taken together with R¹⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl;
or
R¹⁹ and R²⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl;
and
Ring B1, 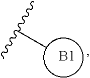
is a five-membered heteroaryl ring containing at least one N, O, or S ring atom, wherein Ring B1 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

3. The compound or tautomer thereof, or pharmaceutically acceptable salt of the compound or tautomer, of claim 1 or claim 2, wherein
A¹¹ is CR¹¹ or N,
A¹² is CR¹² or N,
A¹³ is CR¹³ or N, and
A¹⁴ is CR¹⁴ or N,
wherein no more than two of A¹¹, A¹², A¹³, and A¹⁴ are N;
R¹¹, R¹², R¹³, and R¹⁴ are independently selected from the group consisting of:
H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈
haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,
—NR^{j}R^{k} where R^{j} and R^{k} are independently H, C₁-C₈ alkyl, C₁-C₈ haloalkyl, or C₃-C₈ cycloalkyl, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{k} can be additionally chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{j} and R^{k} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, Ci-C's alkyl, —O—C₁-C₈ alkyl, C₁-C₈ haloalkyl, —C(═O)—C₁-C₈ alkyl, or —O—C₁-C₈ haloalkyl,
a three- to eight-membered heterocyclic ring, a five- to eight-membered heteroaryl ring, a —(C₁-C₄ alkylene)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, or I,
(C₁-C₄ alkylene)-NR^{l}R^{m}, —O—(C₁-C₄ alkylene)-NR^{l}R^{m}, —C(═O)NR^{l}R^{m}, —(C₁-C₄ alkylene)-C(═O)NR^{l}R^{m}, or —O—(C₁-C₄ alkylene)-C(═O)NR^{l}R^{m}, wherein R^{l} and R^{m} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{l} and R^{m} are taken together with the nitrogen to which they are attached to form a four- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three-to eight-membered heterocyclic ring where the carbocyclic or heterocyclic ring is optionally substituted with C₁-C₄ alkyl or OH,
—S(═O)₂—C₁-C₈ alkyl,
—SF₅, and
—S(═O)₂NRⁿR^{o} wherein Rⁿ and R^{o} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or Rⁿ and R^{o} are taken together with the nitrogen to which they are attached to form a four- to eight-membered heterocyclic ring or a five-to eight-membered heteroaryl ring, wherein the heterocyclic ring or heteroaryl ring is optionally substituted with one, two, or three substituents independently selected from the group consisting of —OH, oxo, —CN, C₁-C₈ alkyl, —O—C₁-C₈ alkyl, C₁-C₈ haloalkyl, —O—C₁-C₈ haloalkyl, —C₁-C₈ alkyl-OH, —C(═O)—C₁-C₈ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
Ring C, 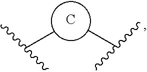
is selected from the group consisting of: 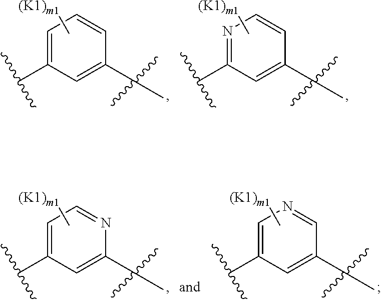
each K1 is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —OH or —O—C₁-C₈ alkyl,
—O—C₁-C₈ alkyl,
—O—C₁-C₈ haloalkyl,
a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K1 groups are taken together with the atoms to which they are attached to form a three- to six-membered carbocyclic or heterocyclic ring, a phenyl ring, or a five- to six-membered heteroaryl ring, wherein the ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —C₁-C₈ haloalkyl, —O—C₁-C₈ alkyl, and —NR^{g1}R^{h1}, where R^{g1} and R^{h1} are independently H or C₁-C₈ alkyl;
m1 is 0, 1, or 2;
R¹⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,
R¹⁸ is selected from the group consisting of H, F, —OH, C₁-C₈ alkyl optionally substituted with —OH, halogen or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl, or
when Y1 is C(R¹⁹)(R²⁰), R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl, or
R¹⁷ and R¹⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and
Y1 is C(R¹⁹)(R²⁰) or S;
R¹⁹ and R²⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or
R¹⁹ can be taken together with R¹⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; and
Ring B1, 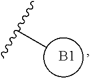
is a five-membered heteroaryl ring containing at least one N, O, or S ring atom, wherein Ring B1 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

4. The compound or tautomer thereof, or pharmaceutically acceptable salt of the compound or tautomer, of any one of claims 1-3, wherein A¹¹ is CR¹¹, A¹² is CR¹², A¹³ is CR¹³, and A¹⁴ is CR¹⁴.

5. The compound or tautomer thereof, or pharmaceutically acceptable salt of the compound or tautomer, of any one of claims 1-3, wherein A¹¹ is CR¹¹, A¹² is N, A¹³ is CR¹³, and A¹⁴ is CR¹⁴.

6. The compound or tautomer thereof, or pharmaceutically acceptable salt of the compound or tautomer, of any one of claims 1-3, wherein A¹¹ is CR¹¹, A¹² is CR¹², A¹³ is N¹³, and A¹⁴ is CR¹⁴.

7. The compound or tautomer thereof, or pharmaceutically acceptable salt of the compound or tautomer, of any one of claims 1-3, wherein A¹¹ is CR¹¹, A¹² is N, A¹³ is N, and A¹⁴ is CR¹⁴.

8. The compound or tautomer thereof, or pharmaceutically acceptable salt of the compound or tautomer, of any one of claims 1-3, wherein:
a) A¹¹ is N, A¹² is CR¹², A¹³ is CR¹³, and A¹⁴ is CR¹⁴;
b) A¹¹ is CR¹¹, A¹² is CR¹², A¹³ is CR¹³, and A¹⁴ is N;
c) A¹¹ is N, A¹² is N, A¹³ is CR¹³, and A¹⁴ is CR¹⁴;
d) A¹¹ is N, A¹² is CR¹², A¹³ is N, and A¹⁴ is CR¹⁴;
e) A¹¹ is N, A¹² is CR¹², A¹³ is CR¹³, and A¹⁴ is N;
f) A¹¹ is CR¹¹, A¹² is N, A¹³ is CR¹³, and A¹⁴ is N; or
g) A¹¹ is CR¹¹, A¹² is CR¹², A¹³ is N, and A¹⁴ is N.

9. The compound or tautomer thereof, or pharmaceutically acceptable salt of the compound or tautomer, of any one of claims 1-8, wherein Ring C, 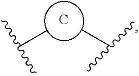
is

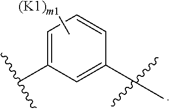

10. The compound or tautomer thereof, or pharmaceutically acceptable salt of the compound or tautomer, of any one of claims 1-9, wherein at least one of R¹¹, R¹², R¹³, and R¹⁴ is selected from the group consisting of: a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

11. The compound or tautomer thereof, or pharmaceutically acceptable salt of the compound or tautomer, of any one of claims 1-9, wherein at least one of R¹¹, R¹², R¹³, and R¹⁴ is selected from the group consisting of: —C₁-C₈ haloalkyl-OH, —C₁-C₈ haloalkyl-COOH, —CO(C₁-C₈ alkyl), a nine-membered heterocyclic ring, —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), and a —O-(four- to eight-membered heterocyclic ring), wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring.

12. The compound or tautomer thereof, or pharmaceutically acceptable salt of the compound or tautomer, of claim 10, wherein R¹² is a —(C₁-C₄ alkylene)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

13. The compound or tautomer thereof, or pharmaceutically acceptable salt of the compound or tautomer, of claim 12, wherein R¹² is a —(C₁-C₂ alkylene)-(four-membered heterocyclic ring), a —(C₁-C₂ alkylene)-(five-membered heterocyclic ring), or a —(C₁-C₂ alkylene)-(six-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

14. The compound or tautomer thereof, or pharmaceutically acceptable salt of the compound or tautomer, of claim 10, wherein R¹² is a —CH₂-(four-to eight-membered heterocyclic ring) or a —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

15. The compound or tautomer thereof, or pharmaceutically acceptable salt of the compound or tautomer, of claim 10, wherein R¹² is —CH₂-(pyrrolidinyl) or —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

16. The compound or tautomer thereof, or pharmaceutically acceptable salt of the compound or tautomer, of claim 1, wherein at least one of R¹¹, R¹², R¹³, and R¹⁴ is selected from the group consisting of: —C₁-C₈ haloalkyl-OH, —C₁-C₈ haloalkyl-COOH, —CO(C₁-C₈ alkyl), a nine-membered heterocyclic ring, —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), and a —O-(four- to eight-membered heterocyclic ring), wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring.

17. The compound or tautomer thereof, or pharmaceutically acceptable salt of the compound or tautomer, of claim 16, wherein R¹² is a —(C₁-C₄ alkylene)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

18. The compound or tautomer thereof, or pharmaceutically acceptable salt of the compound or tautomer, of claim 17, wherein R¹² is a —(C₁-C₂ alkylene)-(four-membered heterocyclic ring), a —(C₁-C₂ alkylene)-(five-membered heterocyclic ring), or a —(C₁-C₂ alkylene)-(six-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

19. The compound or tautomer thereof, or pharmaceutically acceptable salt of the compound or tautomer, of claim 16, wherein R¹² is a —CH₂-(four-to eight-membered heterocyclic ring) or a —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

20. The compound or tautomer thereof, or pharmaceutically acceptable salt of the compound or tautomer, of claim 16, wherein R¹² is —CH₂-(pyrrolidinyl) or —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

21. The compound or tautomer thereof, or pharmaceutically acceptable salt of the compound or tautomer, of claim 10, wherein R¹² is —CH₂-(azetidinyl) or —CH(CH₃)-azetidinyl, wherein the azetidinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

22. The compound or tautomer thereof, or pharmaceutically acceptable salt of the compound or tautomer, of any one of claims 1-21, wherein R¹⁴ is selected from the group consisting of H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl.

23. The compound or tautomer thereof, or pharmaceutically acceptable salt of the compound or tautomer, of any one of claims 1-21, wherein R¹⁴ is CF₃.

24. The compound or tautomer thereof, or pharmaceutically acceptable salt of the compound or tautomer, of any one of claims 1-23, wherein R¹¹, R¹², R¹³, and R¹⁴ are independently selected from the group consisting of
H, F, Cl, Br, I, —CN, C₁-C₈ alkyl, —O—C₁-C₈ alkyl, —C₁-C₈ alkyl-OH, C₁-C₈ haloalkyl, C₃-C₆ cycloalkyl, —S(═O)₂—C₁-C₈ alkyl, —(C₁-C₄ alkylene)-NR^{y}R^{z}, and —C(═O)NR^{y}R^{z},
wherein R^{y} and R^{z} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

25. The compound or tautomer thereof, or pharmaceutically acceptable salt of the compound or tautomer, of any one of claims 1-23, wherein 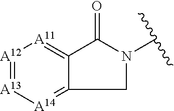
is selected from the group consisting of

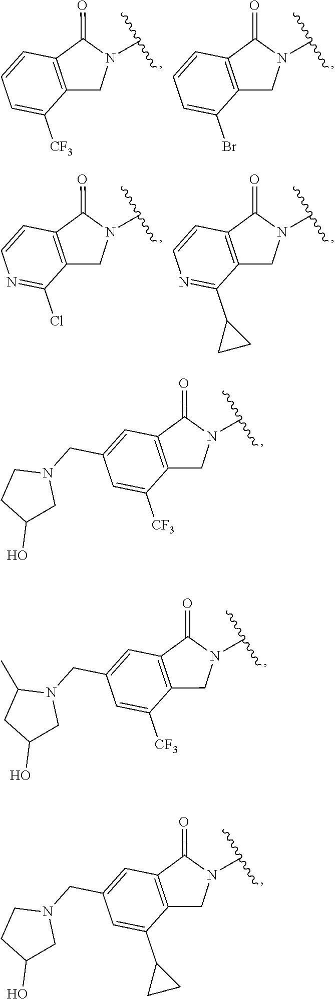

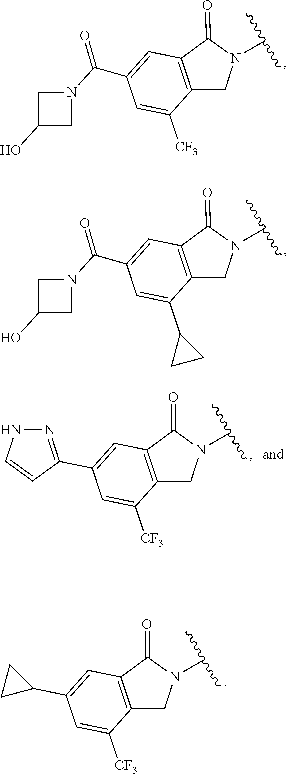

26. The compound or tautomer thereof, or pharmaceutically acceptable salt of the compound or tautomer, of any one of claims 1-23, wherein, 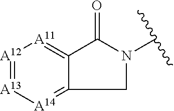
is selected from the group consisting of

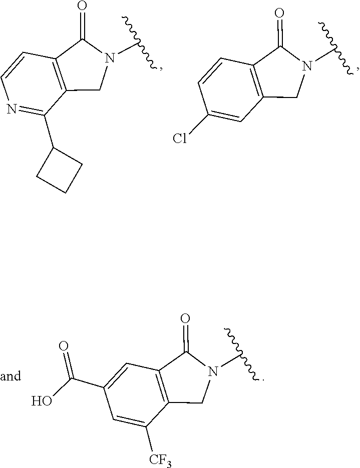 [SMILES: *N1Cc2c(ccnc2C2CCC2)C1=O ; *N1Cc2cc(Cl)ccc2C1=O ; *N1Cc2c(cc(C(=O)O)cc2C(F)(F)F)C1=O]

27. The compound or tautomer thereof, or pharmaceutically acceptable salt of the compound or tautomer, of any one of claims 1-23, wherein 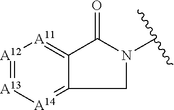
is selected from the group consisting of

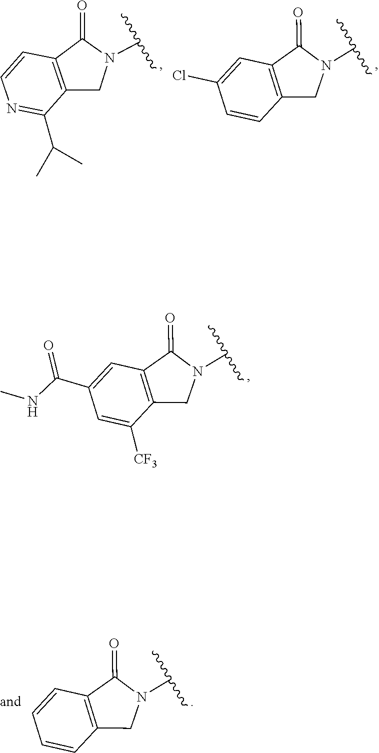 [SMILES: *N1Cc2c(ccnc2C(C)C)C1=O ; *N1Cc2ccc(Cl)cc2C1=O ; *N1Cc2c(cc(C(=O)NC)cc2C(F)(F)F)C1=O ; *N1Cc2ccccc2C1=O]

28. The compound or tautomer thereof, or pharmaceutically acceptable salt of the compound or tautomer, of any one of claims 1-23, wherein 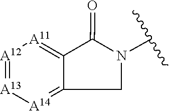
is selected from the group consisting of

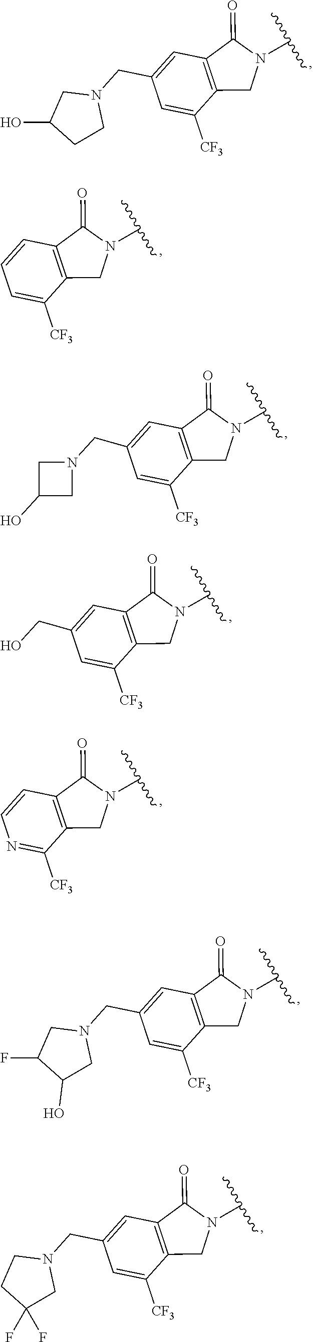

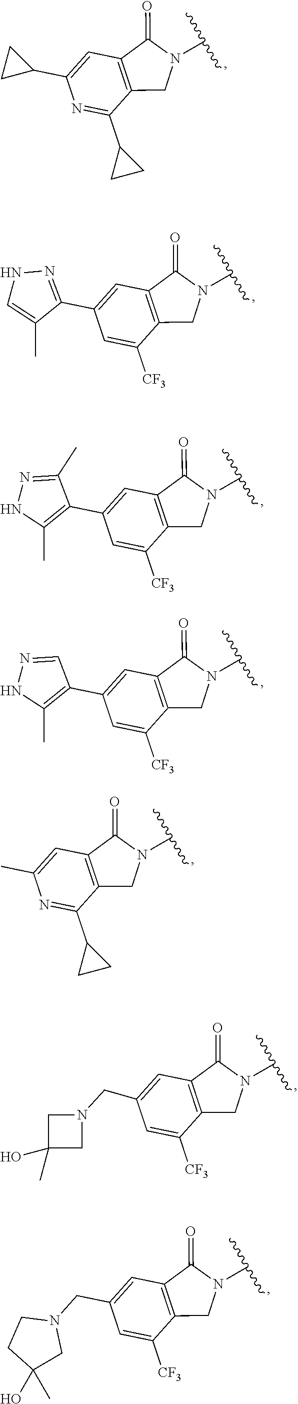

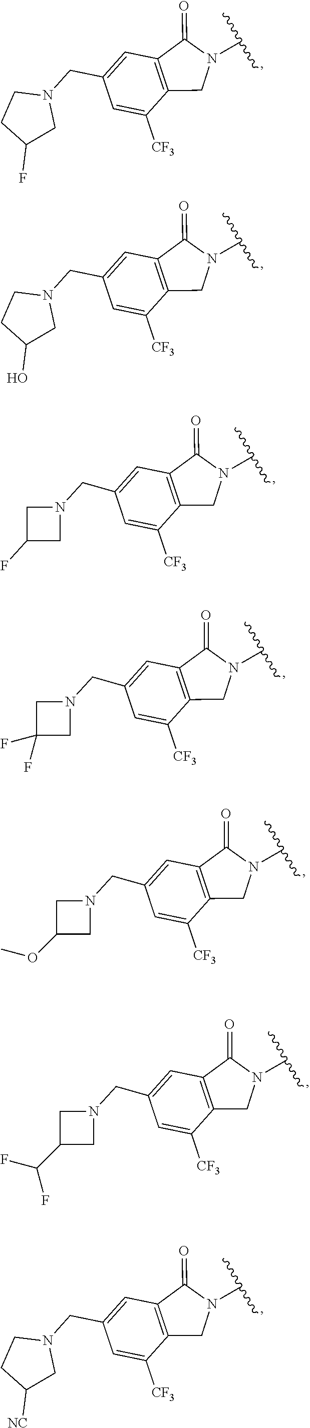

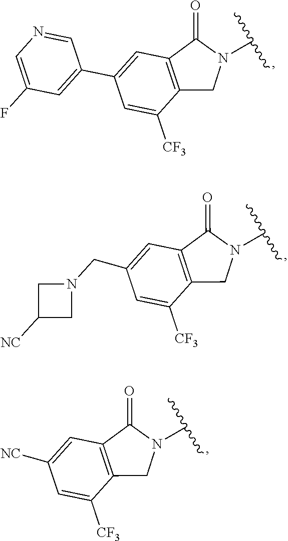 [SMILES: *N1Cc2c(cc(-c3cncc(F)c3)cc2C(F)(F)F)C1=O ; *N1Cc2c(cc(CN3CC(C#N)C3)cc2C(F)(F)F)C1=O ; *N1Cc2c(cc(C#N)cc2C(F)(F)F)C1=O]

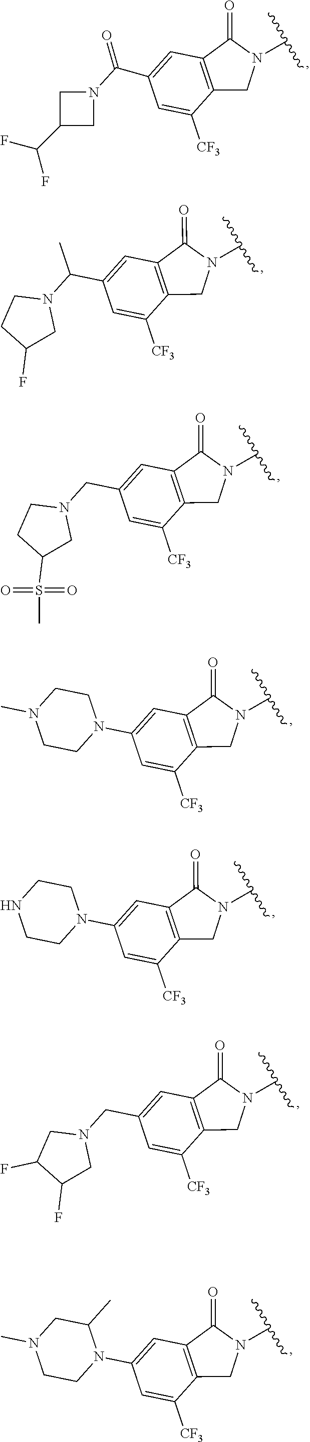

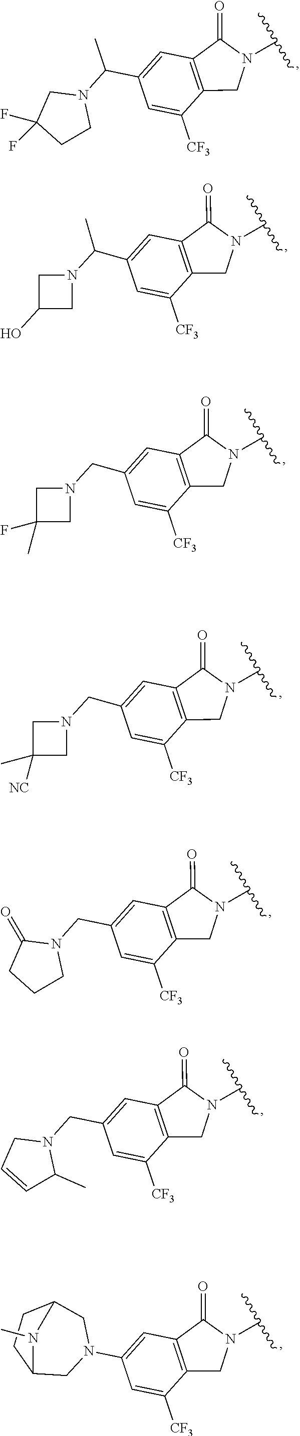

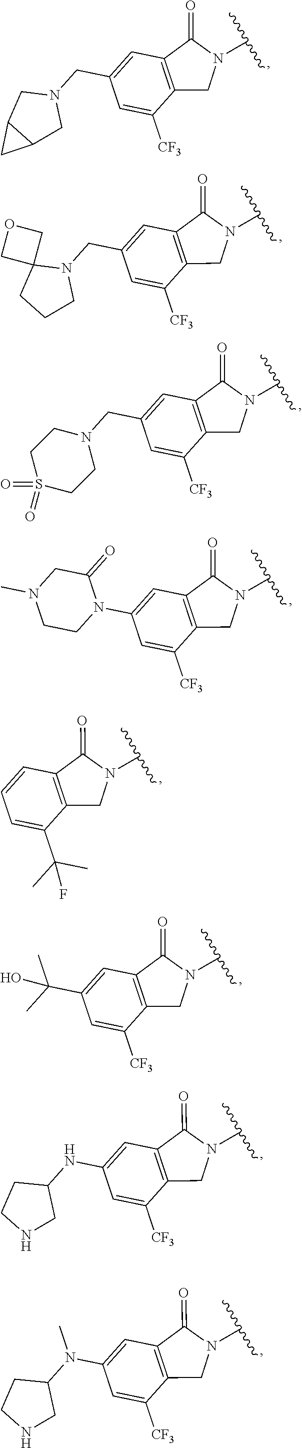

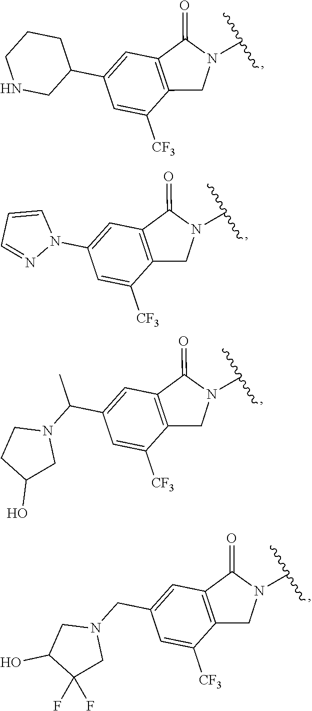

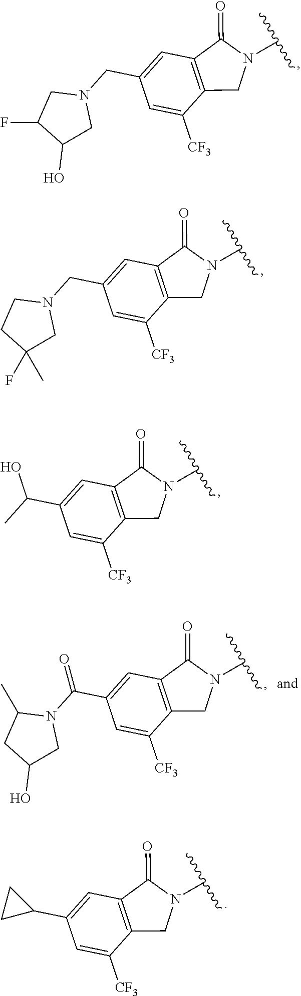 [SMILES: *N1Cc2c(cc(CN3CC(O)C(F)C3)cc2C(F)(F)F)C1=O ; *N1Cc2c(cc(CN3CCC(C)(F)C3)cc2C(F)(F)F)C1=O ; *N1Cc2c(cc(C(C)O)cc2C(F)(F)F)C1=O ; *N1Cc2c(cc(C(=O)N3CC(O)CC3C)cc2C(F)(F)F)C1=O ; *N1Cc2c(cc(C3CC3)cc2C(F)(F)F)C1=O]

29. The compound or tautomer thereof, or pharmaceutically acceptable salt of the compound or tautomer, of any one of claims 1-23, wherein 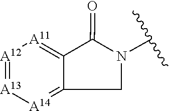
is selected from the group consisting of

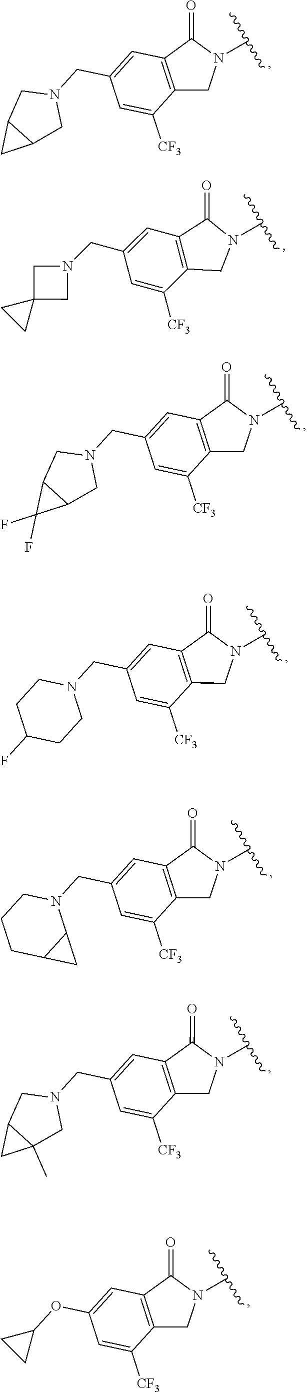

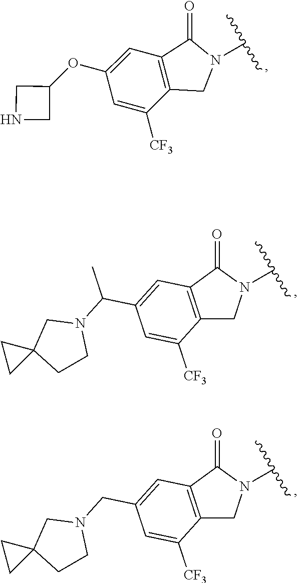 [SMILES: *N1Cc2c(cc(OC3CNC3)cc2C(F)(F)F)C1=O ; *N1Cc2c(cc(C(C)N3CCC4(CC4)C3)cc2C(F)(F)F)C1=O ; *N1Cc2c(cc(CN3CCC4(CC4)C3)cc2C(F)(F)F)C1=O]

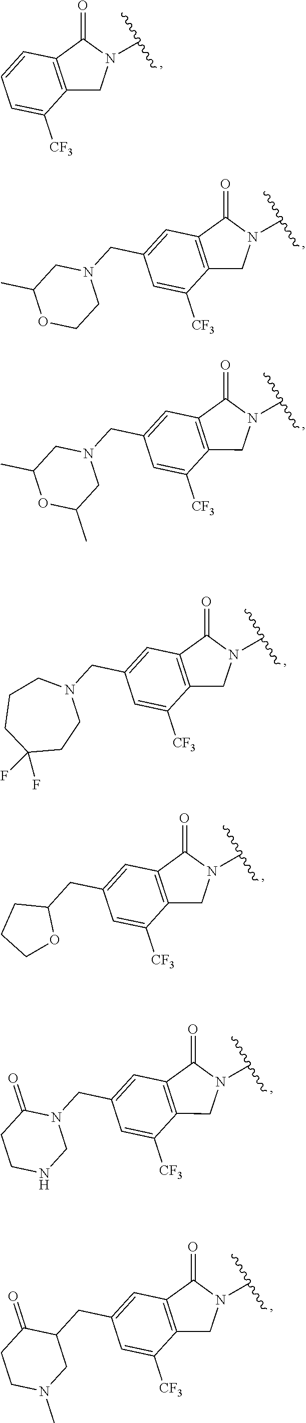

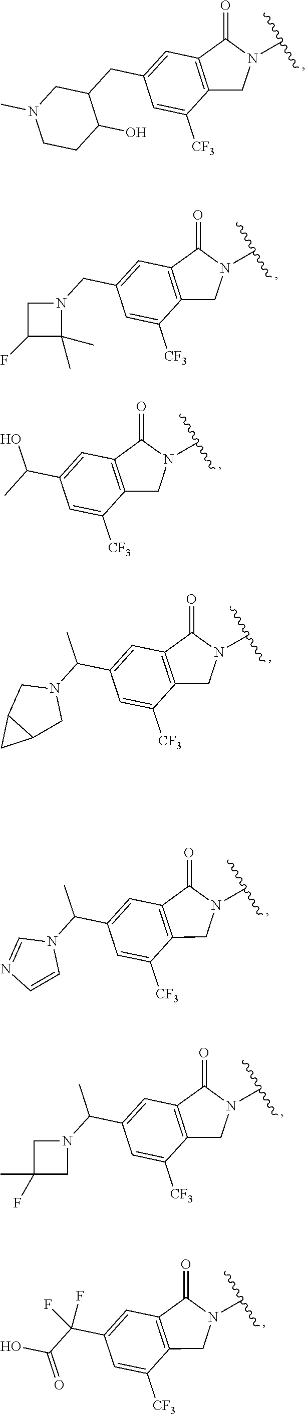

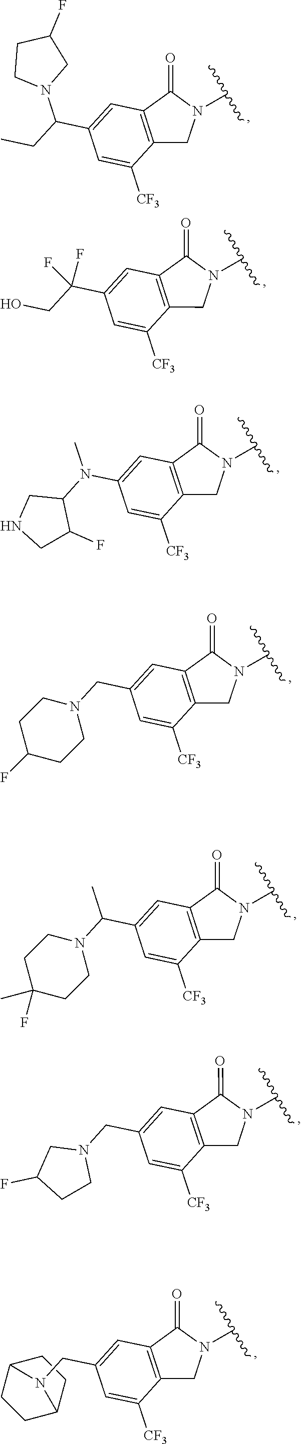

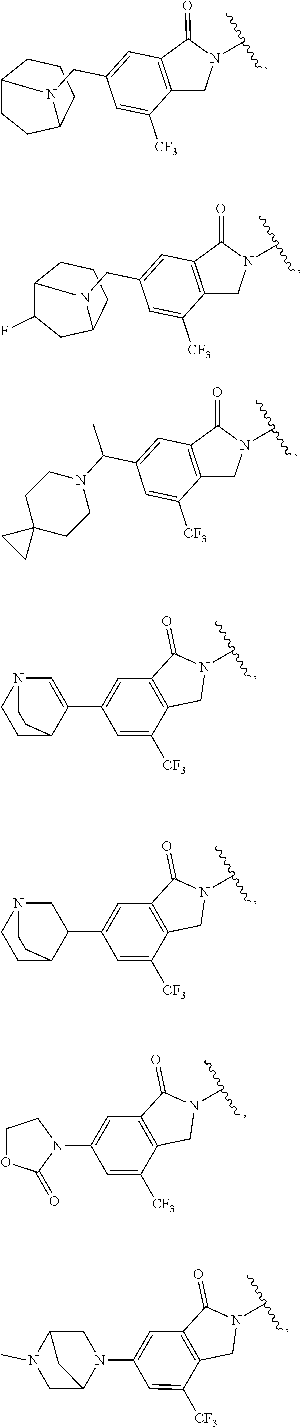

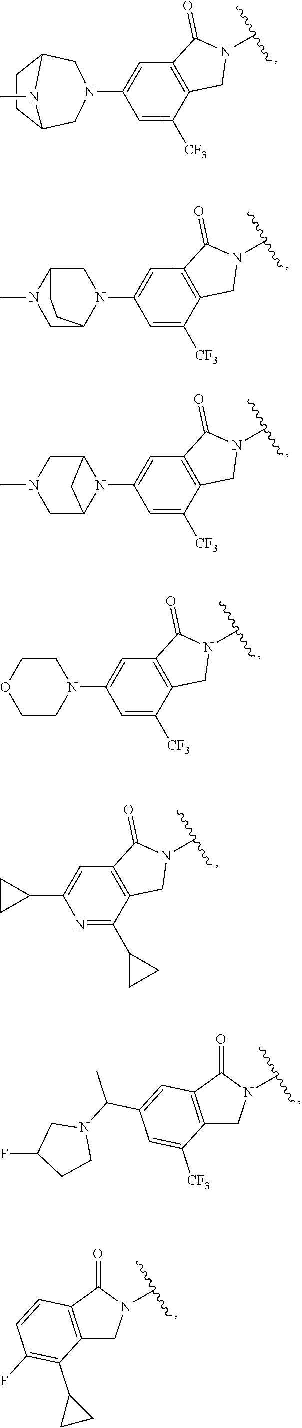

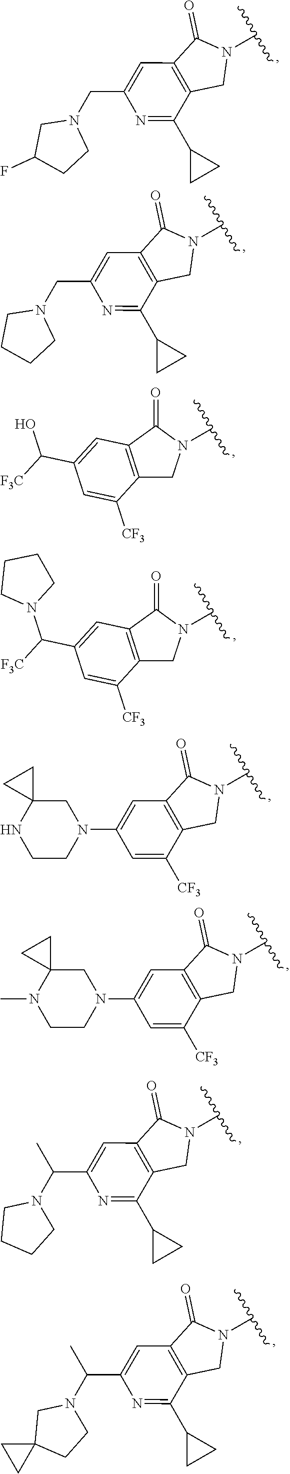

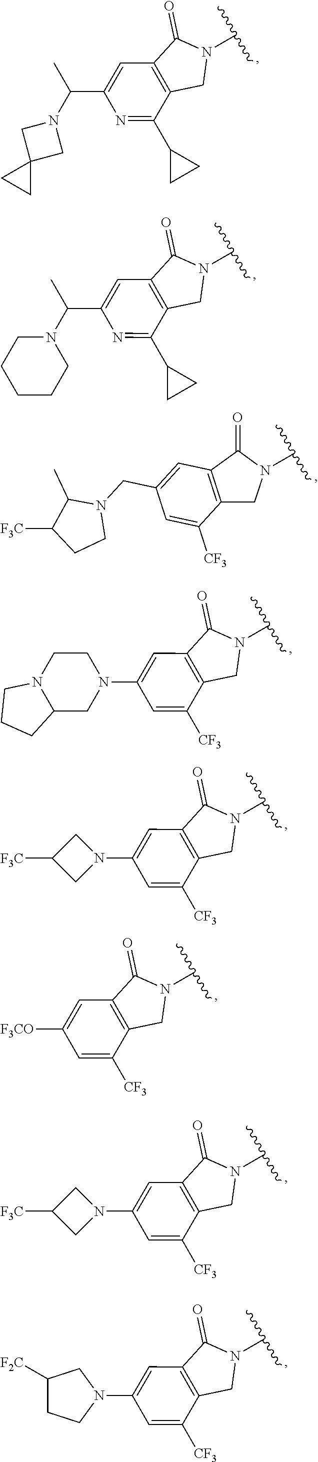

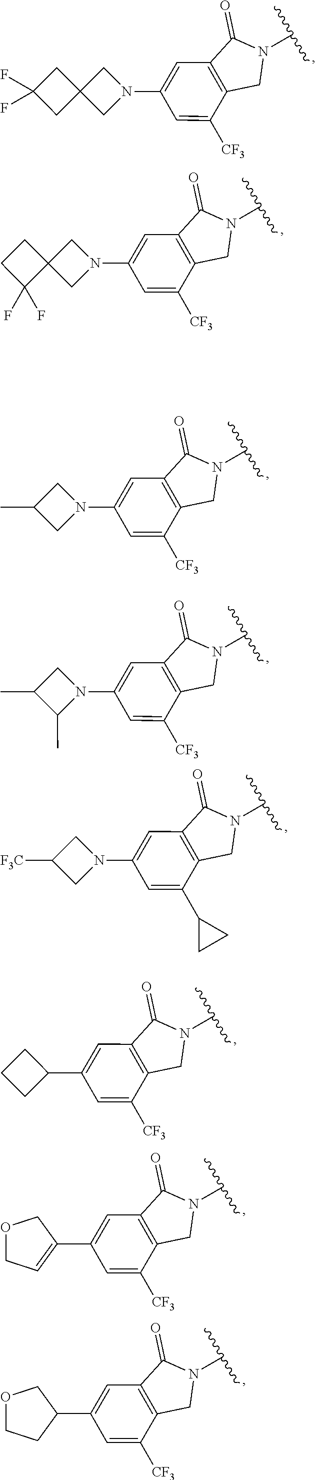

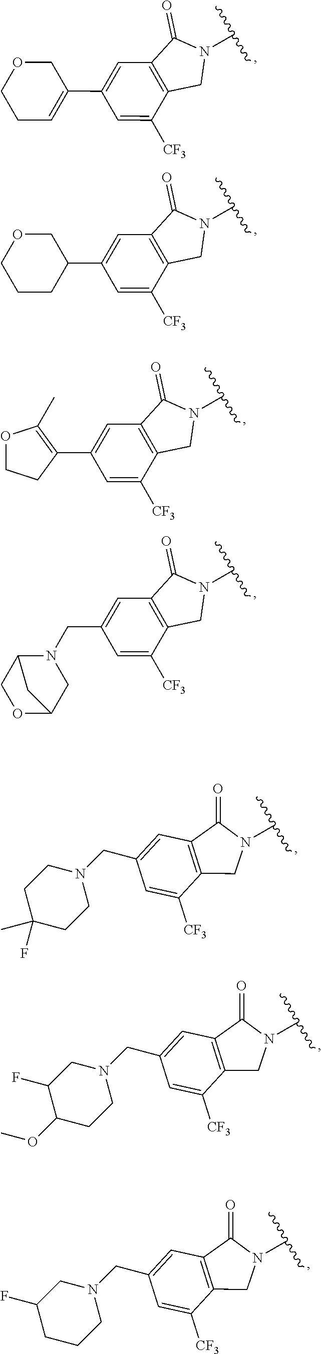

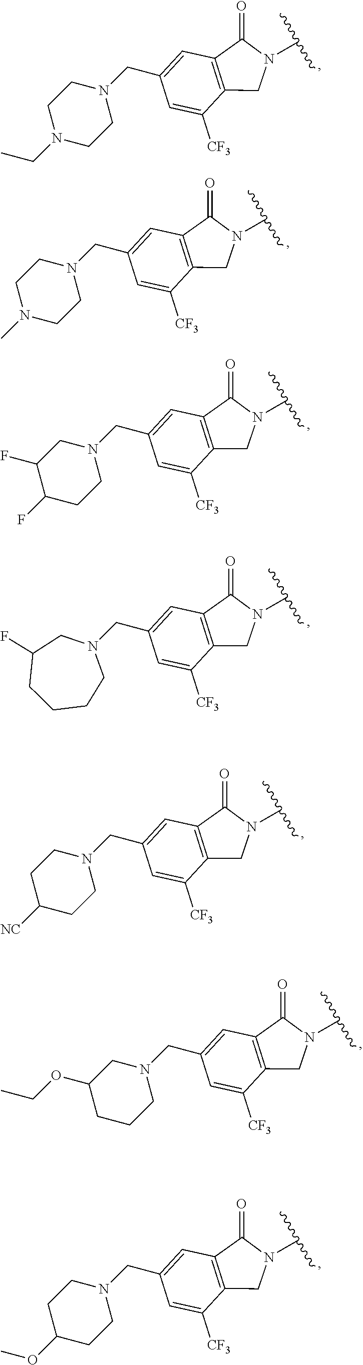

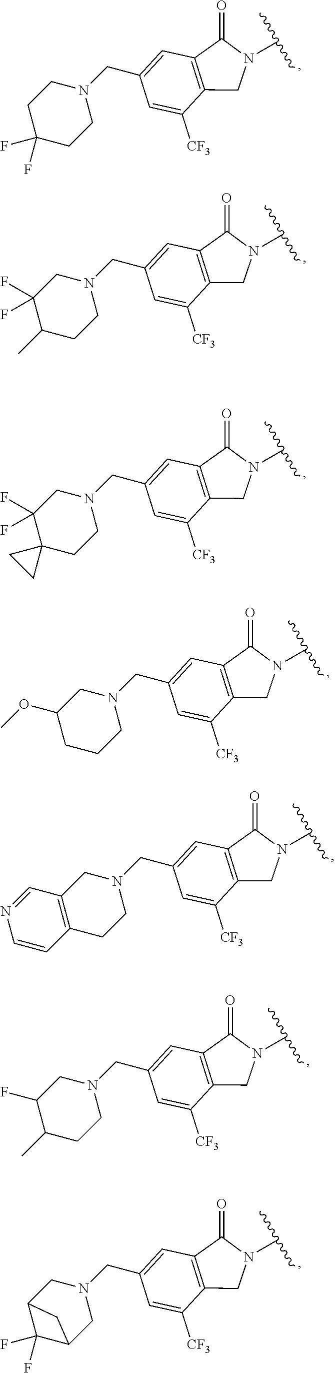

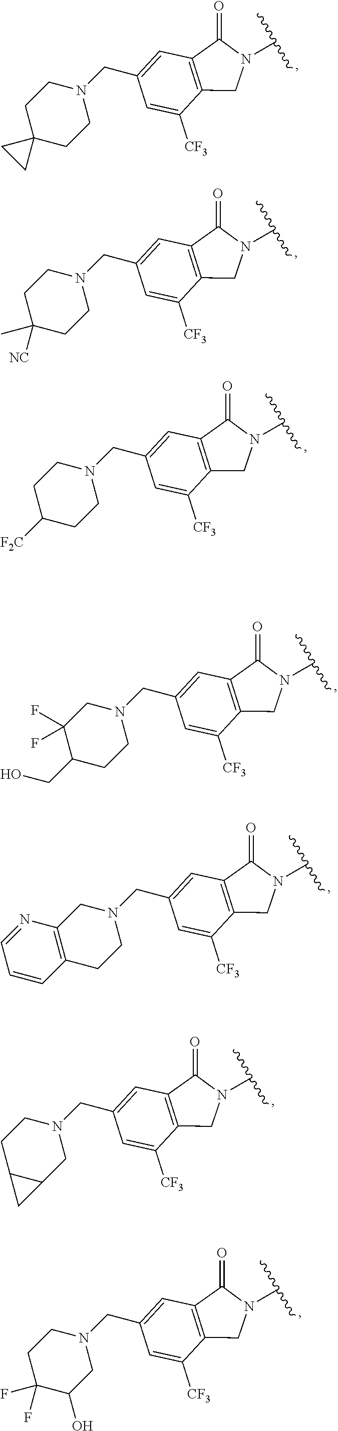

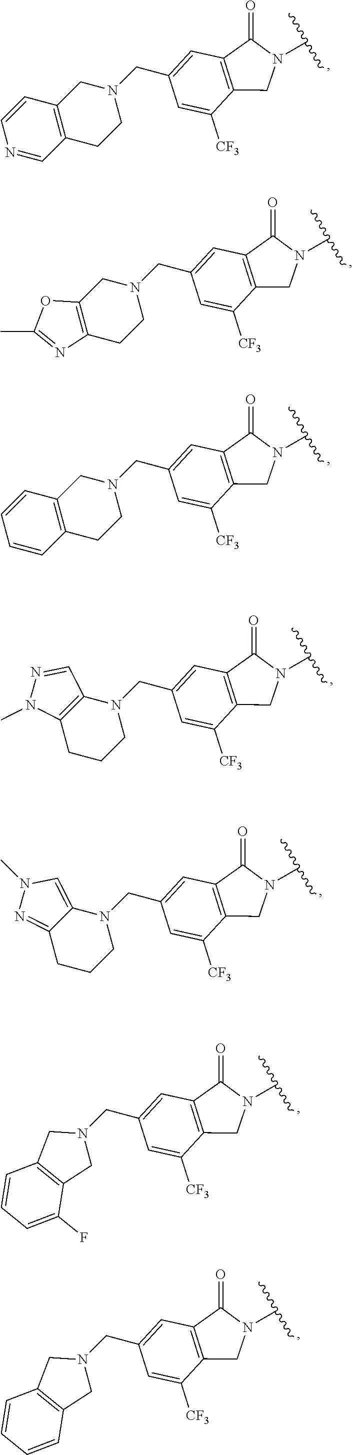

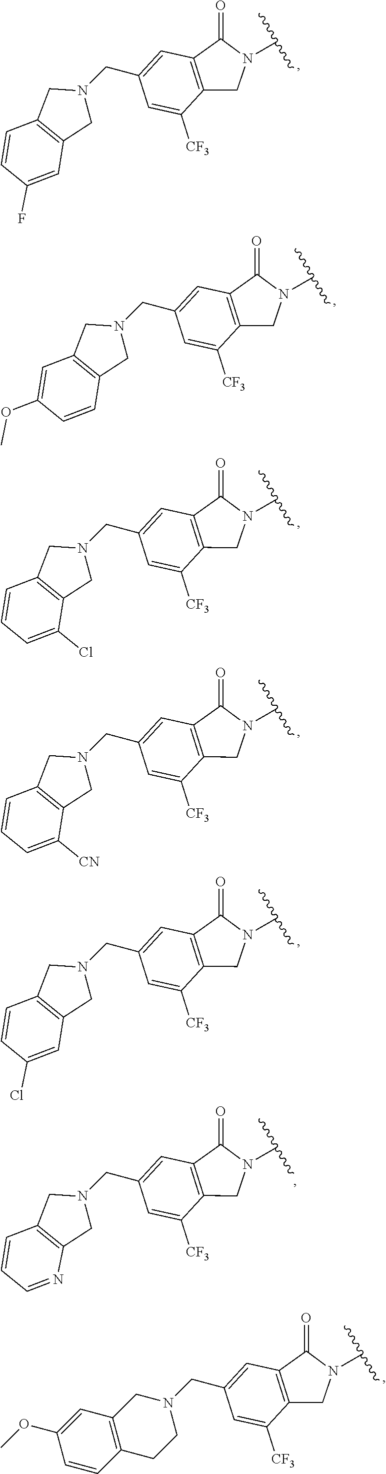

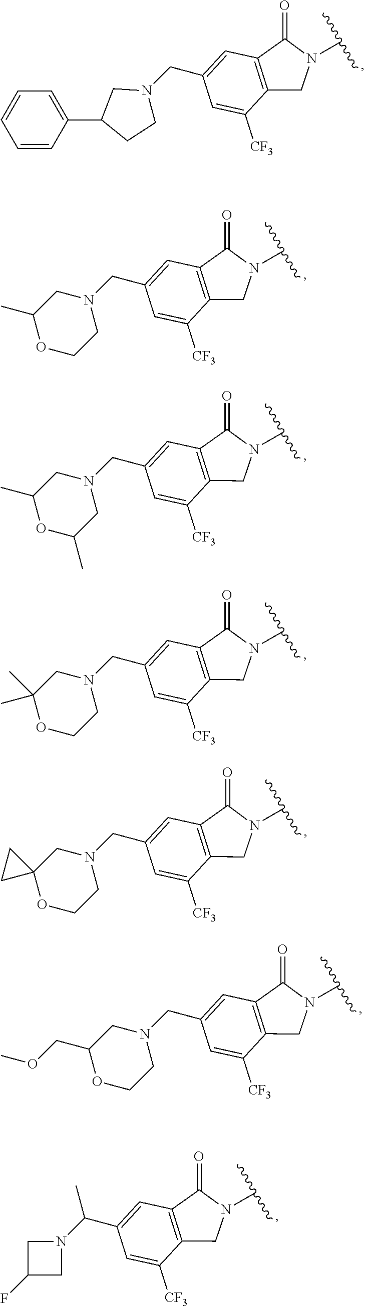

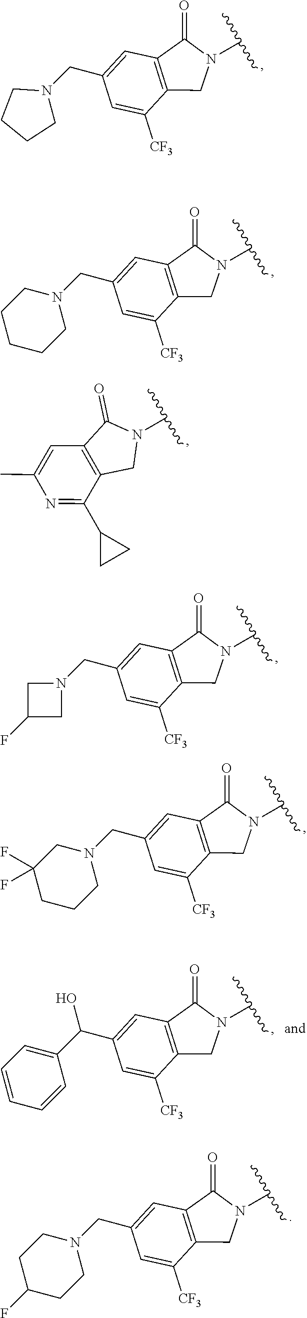

30. The compound or tautomer thereof, or pharmaceutically acceptable salt of the compound or tautomer, of any one of claims 1-29, wherein each K1 is independently selected from the group consisting of:
F, Cl, Br, I, —CN,
C₁-C₈ alkyl optionally substituted with —OH or —O—C₁-C₈ alkyl,
C₃-C₈ cycloalkyl optionally substituted with —OH or —O—C₁-C₈ alkyl,
—O—C₁-C₈ alkyl,
—O—C₁-C₈ haloalkyl, and
—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl.

31. The compound or tautomer thereof, or pharmaceutically acceptable salt of the compound or tautomer, of any one of claims 1-29, wherein each K1 is independently
C₃-C₈ cycloalkyl optionally substituted with —C₁-C₈ alkyl, —OH or —O—C₁-C₈ alkyl, or
—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl.

32. The compound or tautomer thereof, or pharmaceutically acceptable salt of the compound or tautomer, of any one of claims 1-29, wherein two vicinal K1 groups are taken together with the atoms to which they are attached to form a three- to six-membered carbocyclic or heterocyclic ring, a phenyl ring, or a five- to six-membered heteroaryl ring, wherein the ring formed by the two vicinal K1 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

33. The compound or tautomer thereof, or pharmaceutically acceptable salt of the compound or tautomer, of any one of claims 1-29, wherein two vicinal K1 groups are taken together with the atoms to which they are attached to form a five-membered carbocyclic or heterocyclic ring, wherein the five-membered carbocyclic or heterocyclic ring are each optionally substituted with one or two substituents selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

34. The compound or tautomer thereof, or pharmaceutically acceptable salt of the compound or tautomer, of any one of claims 1-33, wherein m1 is 0.

35. The compound or tautomer thereof, or pharmaceutically acceptable salt of the compound or tautomer, of any one of claims 1-33, wherein m1 is 1.

36. The compound or tautomer thereof, or pharmaceutically acceptable salt of the compound or tautomer, of any one of claims 1-35, wherein one of R¹⁷ and R¹⁸ is C₁-C₈ alkyl.

37. The compound or tautomer thereof, or pharmaceutically acceptable salt of the compound or tautomer, of any one of claims 1-35, wherein R¹⁷ is methyl or R¹⁸ is methyl.

38. The compound or tautomer thereof, or pharmaceutically acceptable salt of the compound or tautomer, of any one of claims 1-35, wherein one of R¹⁷ and R¹⁸ is H, F, CF₃ or —CH₂OCH₃.

39. The compound or tautomer thereof, or pharmaceutically acceptable salt of the compound or tautomer, of any one of claims 1-35, wherein one of R¹⁷ and R¹⁸ is methyl and the other is H or F.

40. The compound or tautomer thereof, or pharmaceutically acceptable salt of the compound or tautomer, of any one of claims 1-35, wherein R¹⁷ and R¹⁸ together with the carbon to which they are attached form a C₃-C₈ cycloalkyl ring or three- to six-membered heterocyclyl ring, wherein the C₃-C₈ cycloalkyl ring or the three- to six-membered heterocyclyl ring is optionally substituted with F, Cl, Br, I, —OH, C₁-C₄ alkyl, or —O—C₁-C₄ alkyl.

41. The compound or tautomer thereof, or pharmaceutically acceptable salt of the compound or tautomer, of any one of claims 1-35, wherein R¹⁷ and R¹⁸ together with the carbon to which they are attached form a cyclopropyl or oxetanyl ring, wherein the cyclopropyl or oxetanyl ring is optionally substituted with F, Cl, Br, I, —OH, C₁-C₄ alkyl, or —O—C₁-C₄ alkyl.

42. The compound or tautomer thereof, or pharmaceutically acceptable salt of the compound or tautomer, of any one of claims 1-41, wherein Y1 is C(R¹⁹)(R²⁰), wherein R¹⁹ is selected from the group consisting of H, F, Cl, Br, I, and C₁-C₈ alkyl, and R²⁰ is selected from the group consisting of H, F, Cl, Br, I, and C₁-C₈ alkyl.

43. The compound or tautomer thereof, or pharmaceutically acceptable salt of the compound or tautomer, of claim 42, wherein Y1 is CH₂.

44. The compound or tautomer thereof, or pharmaceutically acceptable salt of the compound or tautomer, of any one of claims 1-41, wherein Y1 is S.

45. The compound or tautomer thereof, or pharmaceutically acceptable salt of the compound or tautomer, of any one of claims 1-35, wherein R¹⁹ is taken together with R¹⁸ to form a three- to six-membered cycloalkyl, heterocyclyl, aryl, or heteroaryl ring, each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

46. The compound or tautomer thereof, or pharmaceutically acceptable salt of the compound or tautomer, of any one of claims 1-35, wherein R¹⁹ is taken together with R¹⁸ to form a three- to six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

47. The compound or tautomer thereof, or pharmaceutically acceptable salt of the compound or tautomer, of any one of claims 1-35, wherein R¹⁹ is taken together with R¹⁸ to form a cyclopropyl, cyclobutyl, cyclopentyl, cyclohexyl, aziridinyl, oxiranyl, thiiranyl, azetidinyl, oxetanyl, thietanyl, pyrrolidinyl, pyrazolidinyl, imidazolidinyl, tetrahydrofuranyl, 1,3-dioxolanyl, tetrahydrothiophenyl, oxathiolanyl, sulfolanyl, piperidinyl, piperazinyl, tetrahydropyranyl, dioxanyl, thianyl, dithianyl, trithianyl, morpholinyl, or thiomorpholinyl ring.

48. The compound or tautomer thereof, or pharmaceutically acceptable salt of the compound or tautomer, of any one of claims 1-35, wherein Y1 is C(R¹⁹)(R²⁰), and the absolute configuration of the carbon atom to which R¹⁷ and R¹⁸ are attached is (R).

49. The compound or tautomer thereof, or pharmaceutically acceptable salt of the compound or tautomer, of any one of claims 1-35, wherein Y1 is C(R¹⁹)(R²⁰), and the absolute configuration of the carbon atom to which R¹⁷ and R¹⁸ are attached is (S).

50. The compound or tautomer thereof, or pharmaceutically acceptable salt of the compound or tautomer, of any one of claims 1-35, wherein Y1 is S, and the absolute configuration of the carbon atom to which R¹⁷ and R¹⁸ are attached is (S).

51. The compound or tautomer thereof, or pharmaceutically acceptable salt of the compound or tautomer, of any one of claims 1-35, wherein Y1 is S, and the absolute configuration of the carbon atom to which R¹⁷ and R¹⁸ are attached is (R).

52. The compound or tautomer thereof, or pharmaceutically acceptable salt of the compound or tautomer, of claim 1, wherein [symbol] is a double bond, Y1 is C(R¹⁹), and Y2 is C(R¹⁸); and R¹⁸ is taken together with R¹⁹ to form a three- to six-membered cycloalkyl, heterocyclyl, or heteroaryl ring or phenyl ring, each optionally substituted with F, Cl, Br, I, —OH, C₁-C₈ alkyl optionally substituted with —OH or —O—C₁-C₈ alkyl, —O—C₁-C₈ alkyl optionally substituted with —OH or —O—C₁-C₈ alkyl, C₁-C₈ haloalkyl, or —O—C₁-C₈ haloalkyl.

53. The compound or tautomer thereof, or pharmaceutically acceptable salt of the compound or tautomer, of any one of claims 1-52, wherein Ring B1 is a five-membered heteroaryl ring containing at least one N, O, or S ring atom, wherein Ring B1 is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

54. The compound or tautomer thereof, or pharmaceutically acceptable salt of the compound or tautomer, of any one of claims 1-52, wherein Ring B1 is a five-membered heteroaryl ring containing at least one N, O, or S ring atom, wherein Ring B1 is optionally substituted with one, two, or three substituents independently selected from the group consisting of F, Cl, Br, or I.

55. The compound or tautomer thereof, or pharmaceutically acceptable salt of the compound or tautomer, of any one of claims 1-52, wherein Ring B1 is a five-membered heteroaryl ring containing at least one N, O, or S ring atom, wherein Ring B1 is optionally substituted with one, two, or three substituents independently selected from the group consisting of methyl, ethyl, cyclopropyl, and —CH₂OH.

56. The compound or tautomer thereof, or pharmaceutically acceptable salt of the compound or tautomer, of any one of claims 1-52, wherein Ring B1 is selected from the group consisting of pyrrole, imidazole, 1,2,4-triazole, 1,2,3-triazole, pyrazole, tetrazole, oxadiazole, oxazole, and isoxazole, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

57. The compound or tautomer thereof, or pharmaceutically acceptable salt of the compound or tautomer, of any one of claims 1-52, wherein Ring B1 is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

58. The compound or tautomer thereof, or pharmaceutically acceptable salt of the compound or tautomer, of any one of claims 1-52, wherein Ring B1 is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with methyl, ethyl, cyclopropyl, or —CH₂OH.

59. The compound or tautomer thereof, or pharmaceutically acceptable salt of the compound or tautomer, of any one of claims 1-52, wherein Ring B1 is selected from the group consisting of

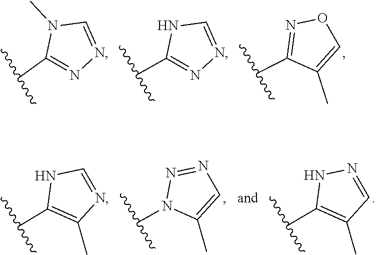

60. The compound or tautomer thereof, or pharmaceutically acceptable salt of the compound or tautomer, of any one of claims 1-52, wherein Ring B1 is selected from the group consisting of

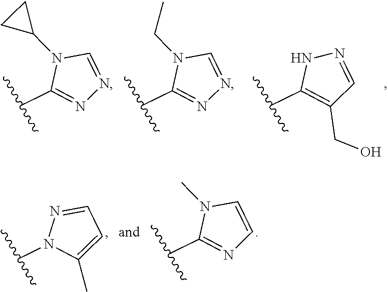

61. The compound or tautomer thereof, or pharmaceutically acceptable salt of the compound or tautomer, of any one of claims 1-52, wherein Ring B1 is selected from the group consisting of

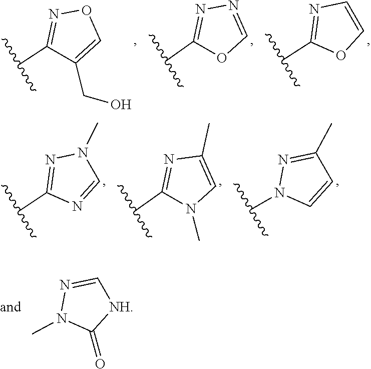

62. The compound or tautomer thereof, or pharmaceutically acceptable salt of the compound or tautomer, of any one of claims 1-52, wherein Ring B1 is selected from the group consisting of

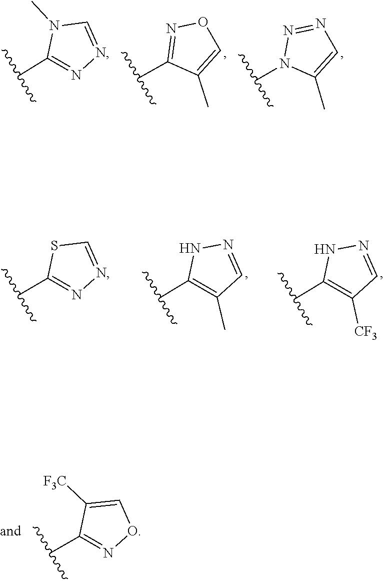

63. The compound or tautomer thereof, or pharmaceutically acceptable salt of the compound or tautomer, of any one of claims 1-52, wherein Ring B1 is 4-methyl-4H-1,2,4-triazol-3-yl.

64. The compound or tautomer thereof, or pharmaceutically acceptable salt of the compound or tautomer, of claim 1, selected from the group consisting of Compounds 86, 162-169, 171-180, 255a-283b, 289-301, and 304a-304b of Table 1, including “a” and “b” variants of the compounds, tautomers thereof, and pharmaceutically acceptable salts of the compounds or tautomers.

65. The compound or tautomer thereof, or pharmaceutically acceptable salt of the compound or tautomer, of claim 1, which is compound 255a, a tautomer thereof, or pharmaceutically acceptable salt of the compound or tautomer.

66. The compound or tautomer thereof, or pharmaceutically acceptable salt of the compound or tautomer, of claim 1, which is compound 282, a tautomer thereof, or pharmaceutically acceptable salt of the compound or tautomer.

67. The compound or tautomer thereof, or pharmaceutically acceptable salt of the compound or tautomer, of claim 1, which is a compound selected from the group consisting of

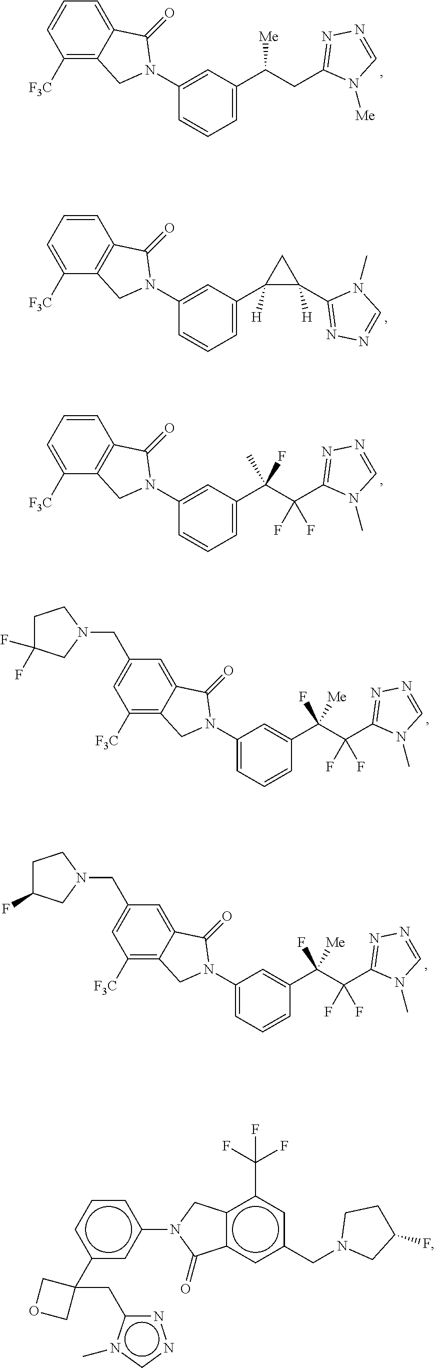

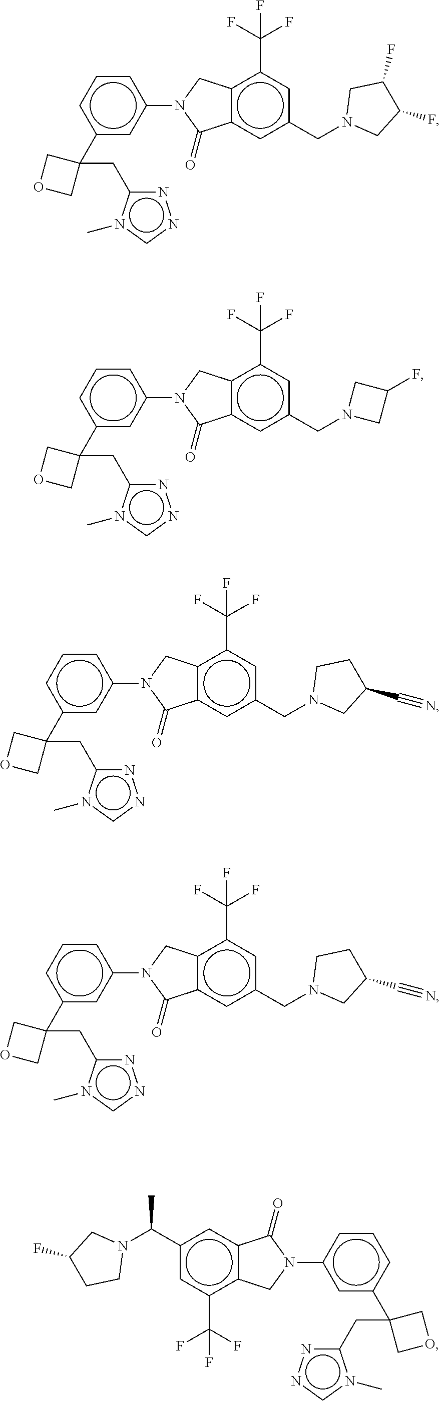

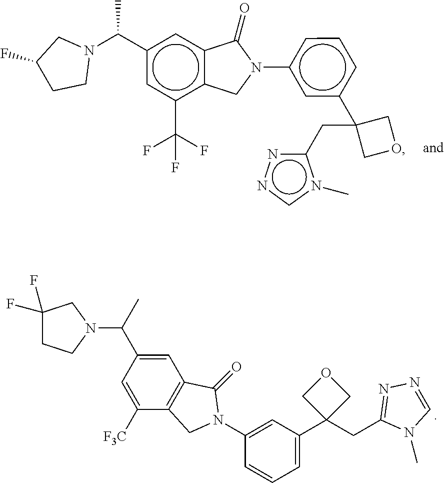

68. A compound of Formula (II-A): 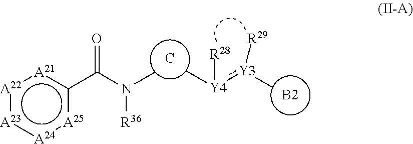
or a tautomer thereof, or a pharmaceutically acceptable salt of any of the foregoing,
wherein
A²¹ is CR²¹ or N, or is absent,
A²² is CR²² or N,
A²³ is CR²³ or N,
A²⁴ is CR²⁴ or N, and
A²⁵ is CR²⁵ or N,
wherein no more than two of A²¹, A²², A²³, A²⁴, and A²⁵ are N;
R²¹, R²², R²³, and R²⁴ are independently selected from R^{x};
each R^{x} is independently selected from the group consisting of:
H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,
—NR^{p}R^{q} where R^{p} and R^{q} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{q} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{p} and R^{q} are taken together with the nitrogen to which they are attached to form a four- to eight-membered heterocyclic ring or a five-to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a —C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring,
—(C₁-C₄ alkylene)-NR^{r}R^{s}, —O—(C₁-C₄ alkylene)-NR^{r}R^{s}, —C(═O)NR^{r}R^{s}, —(C₁-C₄ alkylene)-C(═O)NR^{r}R^{s}, or —O—(C₁-C₄ alkylene)-C(═O)NR^{r}R^{s}, wherein R^{r} and R^{s} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{r} and R^{s} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three-to eight-membered heterocyclic ring where the carbocyclic or heterocyclic ring is optionally substituted with C₁-C₄ alkyl or OH;
—S(═O)₂—C₁-C₈ alkyl,
—SF₅, and
—S(═O)₂NR^{t}R^{u} wherein R^{t} and R^{u} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{t} and R^{u} are taken together with the nitrogen to which they are attached to form a three- to eight-membered heterocyclic ring or a five-to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
R²⁵ is independently selected from R^{x}, and R²⁶ is H; or
A₂₅ is CR²⁵, and R²⁵, R²⁶ and the intervening atoms are taken together to form a five-membered lactam ring, such that the fragment 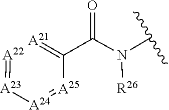
is 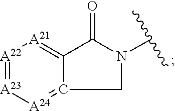
or
(R²¹ and R²²) or (R²² and R²³) or (R²³ and R²⁴) or (R²⁴ and R²⁵), together with the atoms to which they are attached, are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl);
Ring C, 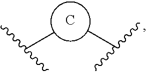
is selected from the group consisting of: 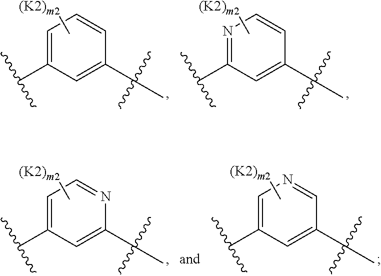
each K2 is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —C₁-C₈ alkyl, —OH or —O—C₁-C₈ alkyl,
—O—C₁-C₈ alkyl optionally substituted with —OH,
—O—C₁-C₈ haloalkyl,
—O-(three- to six-membered heterocyclic ring) optionally substituted with C₁-C₈ alkyl,
a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K2 groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;
m2 is 0, 1, or 2; [symbol]
is a single bond or a double bond,
wherein when [symbol] is a single bond, Y3 is C(R³⁰) and Y4 is C(R²⁷); and
when [symbol] is a double bond, Y3 is C and Y4 is C;
R²⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl;
R²⁸ and R²⁹, as indicated by the dashed curve [symbol] , are taken together with the atoms to which they are attached to form a three- to six-membered cycloalkyl, heterocyclyl, aryl, or heteroaryl ring, wherein the three- to six-membered cycloalkyl, heterocyclyl, aryl, or heteroaryl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl,
R³⁰ is selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; and
Ring B2, 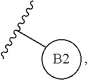
is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

69. The compound of claim 68, wherein the compound of Formula (II-A) is a compound of Formula (II): 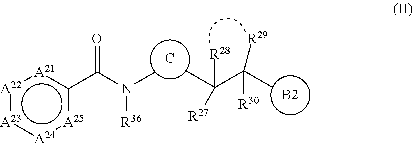
or a tautomer thereof, or a pharmaceutically acceptable salt of any of the foregoing,
wherein
A²¹ is CR²¹ or N, or is absent,
A²² is CR²² or N,
A²³ is CR²³ or N,
A²⁴ is CR²⁴ or N, and
A²⁵ is CR²⁵ or N,
wherein no more than two of A²¹, A²², A²³, A²⁴, and A²⁵ are N;
R²¹, R²², R²³, and R²⁴ are independently selected from R^{x};
each R^{x} is independently selected from the group consisting of:
H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂,
—C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,
—NR^{p}R^{q} where R^{p} and R^{q} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{q} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{p} and R^{q} are taken together with the nitrogen to which they are attached to form a four- to eight-membered heterocyclic ring or a five-to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, CN, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring,
(C₁-C₄ alkylene)-NR^{r}R^{s}, —O—(C₁-C₄ alkylene)-NR^{r}R^{s}, —C(═O)NR^{r}R^{s}, —(C₁-C₄ alkylene)-C(═O)NR^{r}R^{s}, or —O—(C₁-C₄ alkylene)-C(═O)NR^{r}R^{s}, wherein R^{r} and R^{s} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{r} and R^{s} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three-to eight-membered heterocyclic ring where the carbocyclic or heterocyclic ring is optionally substituted with C₁-C₄ alkyl or OH;
—S(═O)₂—C₁-C₈ alkyl,
—SF₅, and
—S(═O)₂NR^{t}R^{u} wherein R and R^{u} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{t} and R^{u} are taken together with the nitrogen to which they are attached to form a three- to eight-membered heterocyclic ring or a five-to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
R²⁵ is independently selected from R^{x}, and R²⁶ is H; or
A₂₅ is CR²⁵, and R²⁵, R²⁶ and the intervening atoms are taken together to form a five-membered lactam ring, such that the fragment 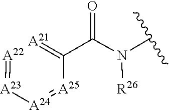
is 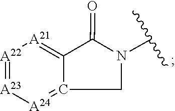
or
(R²¹ and R²²) or (R²² and R²³) or (R²³ and R²⁴) or (R²⁴ and R²⁵), together with the atoms to which they are attached, are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl);
Ring C, 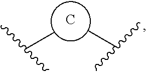
is selected from the group consisting of: 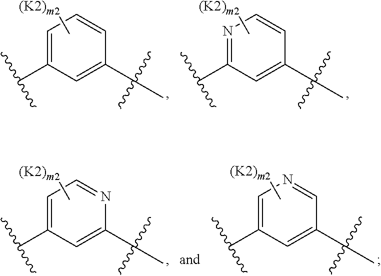
each K2 is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —OH or —O—C₁-C₈ alkyl,
—O—C₁-C₈ alkyl optionally substituted with —OH,
—O—C₁-C₈ haloalkyl,
—O-(three- to six-membered heterocyclic ring) optionally substituted with C₁-C₈ alkyl, a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, and four- to eight-membered heterocyclyl optionally substituted with —OH or C₁-C₄ alkyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K2 groups are taken together with the atoms to which they are attached to form a three- to six-membered carbocyclic or heterocyclic ring, a phenyl ring, or a five- to six-membered heteroaryl ring, wherein the ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;
m2 is 0, 1, or 2;
R²⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl;
R²⁸ and R²⁹, as indicated by the dashed curve [symbol] are taken together with the atoms to which they are attached to form a three- to six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl,
R³⁰ is selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; and
Ring B2, 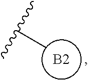
is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

70. The compound or tautomer thereof, or pharmaceutically acceptable salt of the compound or tautomer, of claim 68 or claim 69,
wherein
A²¹ is CR²¹ or N, or is absent,
A²² is CR²² or N,
A²³ is CR²³ or N,
A²⁴ is CR²⁴ or N, and
A²⁵ is CR²⁵ or N,
wherein no more than two of A²¹, A²², A²³, A²⁴, and A²⁵ are N;
R²¹, R²², R²³, and R²⁴ are independently selected from R^{x};
each R^{x} is independently selected from the group consisting of:
H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,
—NR^{p}R^{q} where R^{p} and R^{q} are independently H, C₁-C₈ alkyl, C₁-C₈ haloalkyl, or C₃-C₈ cycloalkyl, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{q} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{p} and R^{q} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to eight-membered heterocyclic ring, a five- to eight-membered heteroaryl ring, a —(C₁-C₄ alkylene)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, or I,
(C₁-C₄ alkylene)-NR^{r}R^{s}, —O—(C₁-C₄ alkylene)-NR^{r}R^{s}, —C(═O)NR^{r}R^{s}, —(C₁-C₄ alkylene)-C(═O)NR^{r}R^{s}, or —O—(C₁-C₄ alkylene)-C(═O)NR^{r}R^{s}, wherein R^{r} and R^{s} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{r} and R^{s} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
—S(═O)₂—C₁-C₈ alkyl,
—SF₅, and
—S(═O)₂NR^{t}R^{u} wherein R and R^{u} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{t} and R^{u} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
R²⁵ is independently selected from R^{x}, and R²⁶ is H; or
A₂₅ is CR²⁵, and R²⁵, R²⁶ and the intervening atoms are taken together to form a five-membered lactam ring, such that the fragment 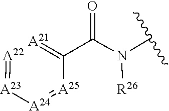
is 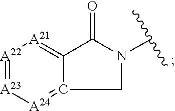
or
(R²¹ and R²²) or (R²² and R²³) or (R²³ and R²⁴) or (R²⁴ and R²⁵), together with the atoms to which they are attached, are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl);
Ring C, 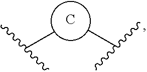
is selected from the group consisting of: 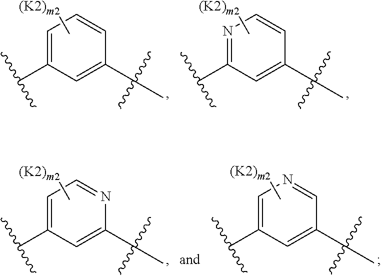
each K2 is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —OH or —O—C₁-C₈ alkyl,
—O—C₁-C₈ alkyl,
—O—C₁-C₈ haloalkyl,
a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K2 groups are taken together with the atoms to which they are attached to form a three- to six-membered carbocyclic or heterocyclic ring, a phenyl ring, or a five- to six-membered heteroaryl ring, wherein the ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl; and
m2 is 0, 1, or 2;
R²⁷ is selected from the group consisting of H, F, —OH, C₁-C₈ alkyl optionally substituted with —OH or —O—C₁-C₈ alkyl, C₁-C₈ alkenyl, C₁-C₈ haloalkyl, —O—C₁-C₈ haloalkyl, and —O—C₁-C₈ alkyl optionally substituted with —OH or —O—C₁-C₈ alkyl;
R²⁸ and R²⁹, as indicated by the dashed curve [symbol] , are taken together with the atoms to which they are attached to form a three- to six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl;
R³⁰ is selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; and
Ring B2, 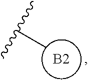
is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

71. The compound or tautomer thereof, or pharmaceutically acceptable salt of the compound or tautomer, of any one of claims 68-70, wherein A²¹ is CR²¹ or N.

72. The compound or tautomer thereof, or pharmaceutically acceptable salt of the compound or tautomer, of any one of claims 68-71, wherein Ring C, 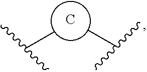
is

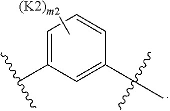

73. The compound or tautomer thereof, or pharmaceutically acceptable salt of the compound or tautomer, of any one of claims 68-72, wherein R²¹, R²², R²³, and R²⁴ are independently selected from the group consisting of
F, Cl, Br, I, —CN, C₁-C₈ alkyl, —O—C₁-C₈ alkyl, —C₁-C₈ alkyl-OH, C₁-C₈ haloalkyl, C₃-C₆ cycloalkyl, —S(═O)₂—C₁-C₈ alkyl, —(C₁-C₄ alkylene)-NR^{r}R^{s}, and —C(═O)NR^{r}R^{s},
wherein R^{r} and R^{s} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

74. The compound or tautomer thereof, or pharmaceutically acceptable salt of the compound or tautomer, of any one of claims 68-73, wherein R²¹ and R²², together with the atoms to which they are attached are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl).

75. The compound or tautomer thereof, or pharmaceutically acceptable salt of the compound or tautomer, of any one of claims 68-72, wherein R²² and R²³, together with the atoms to which they are attached are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl).

76. The compound or tautomer thereof, or pharmaceutically acceptable salt of the compound or tautomer, of any one of claims 68-72, wherein R²³ and R²⁴, together with the atoms to which they are attached are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl).

77. The compound or tautomer thereof, or pharmaceutically acceptable salt of the compound or tautomer, of any one of claims 68-72, wherein R²⁴ and R²⁵, together with the atoms to which they are attached are taken together to form a five-membered or six-membered carbocyclic, heterocyclic, aryl, or heteroaryl ring optionally substituted with —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl).

78. The compound or tautomer thereof, or pharmaceutically acceptable salt of the compound or tautomer, of any one of claims 68-72, wherein 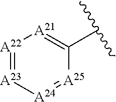
is selected from the group consisting of

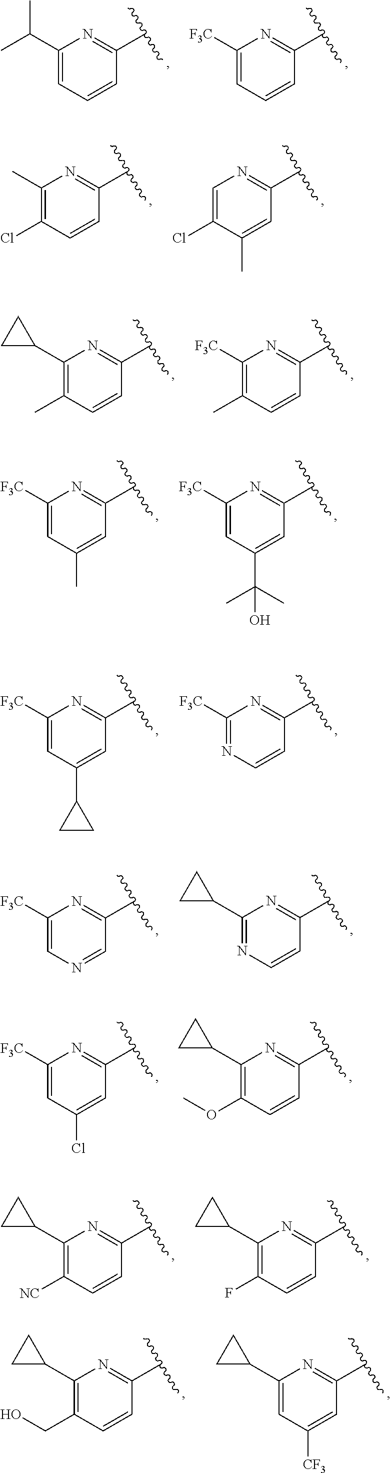

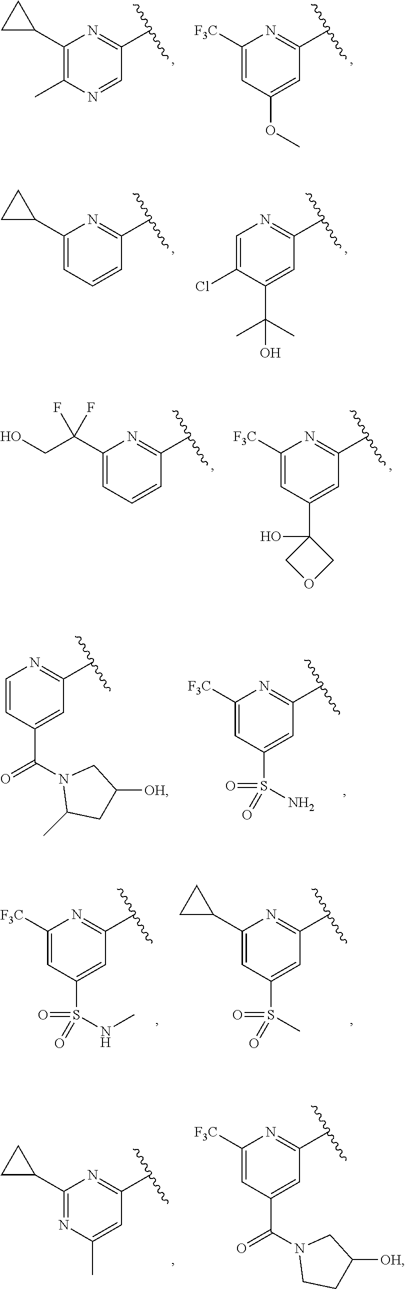

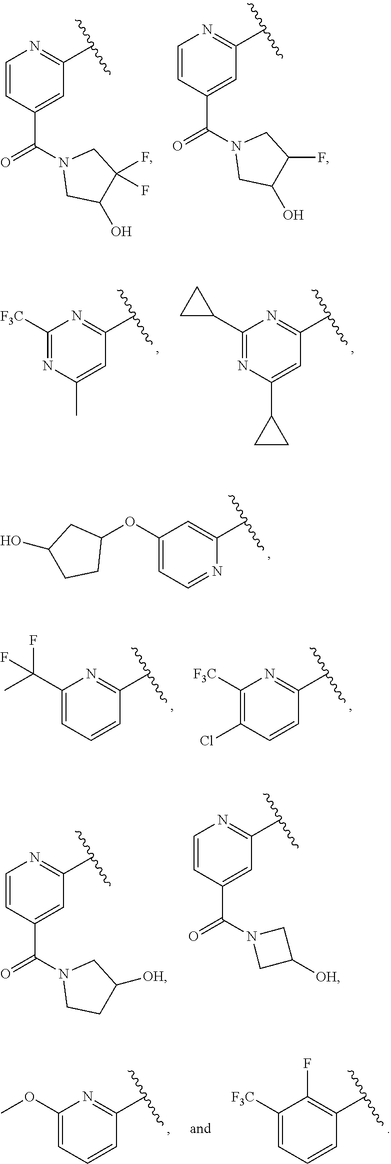

79. The compound or tautomer thereof, or pharmaceutically acceptable salt of the compound or tautomer, of any one of claims 68-72, wherein 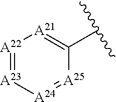
is selected from the group consisting of

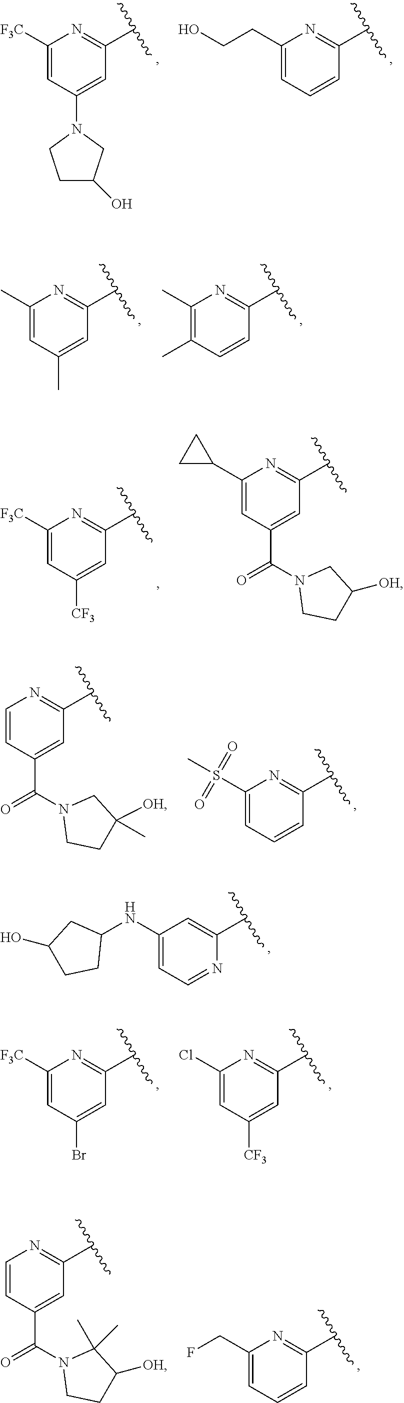

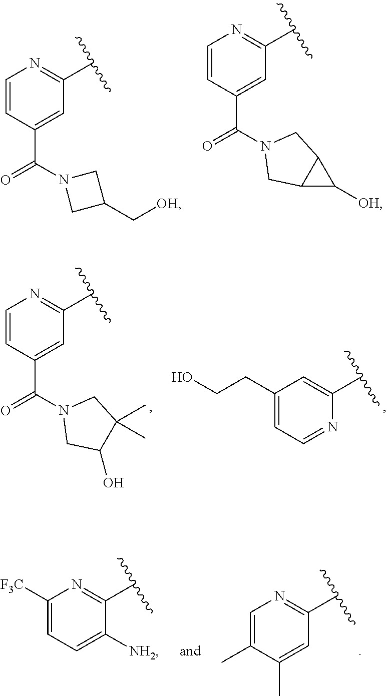

80. The compound or tautomer thereof, or pharmaceutically acceptable salt of the compound or tautomer, of any one of claims 68-72, wherein 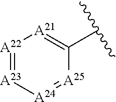
is selected from the group consisting of

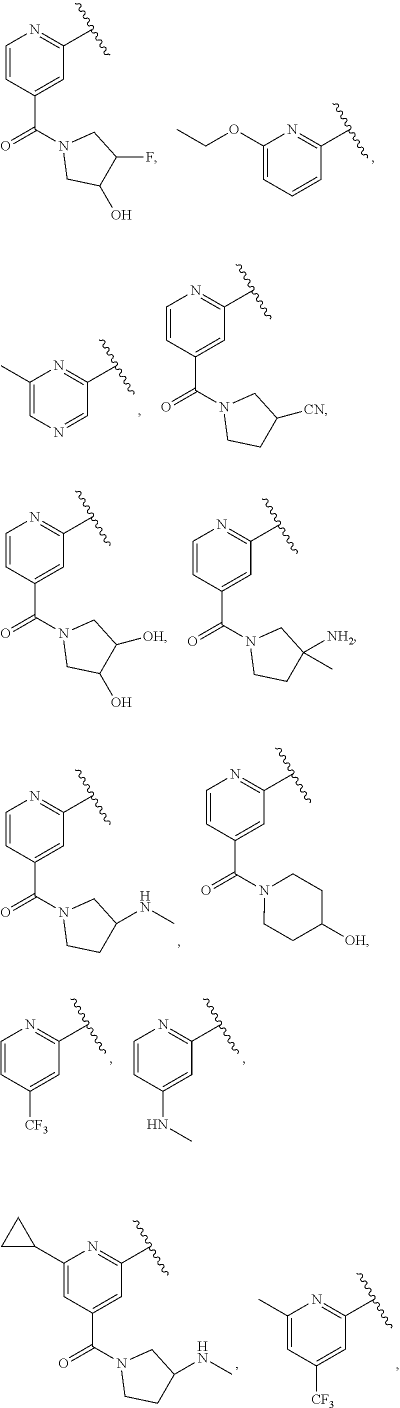

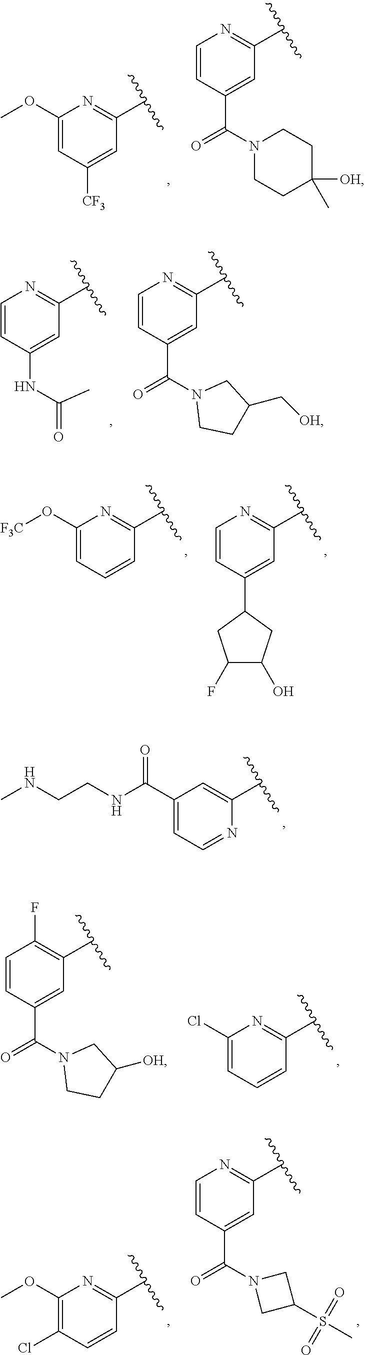

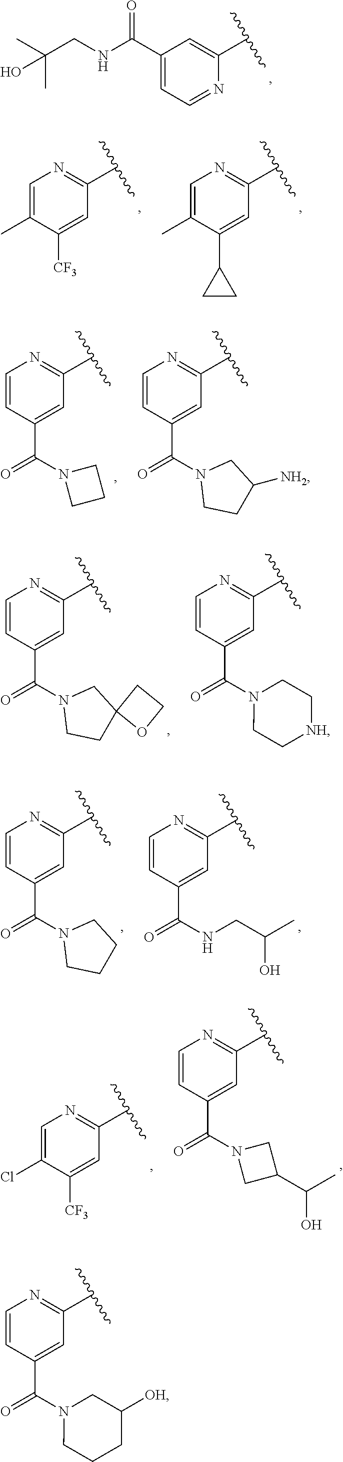

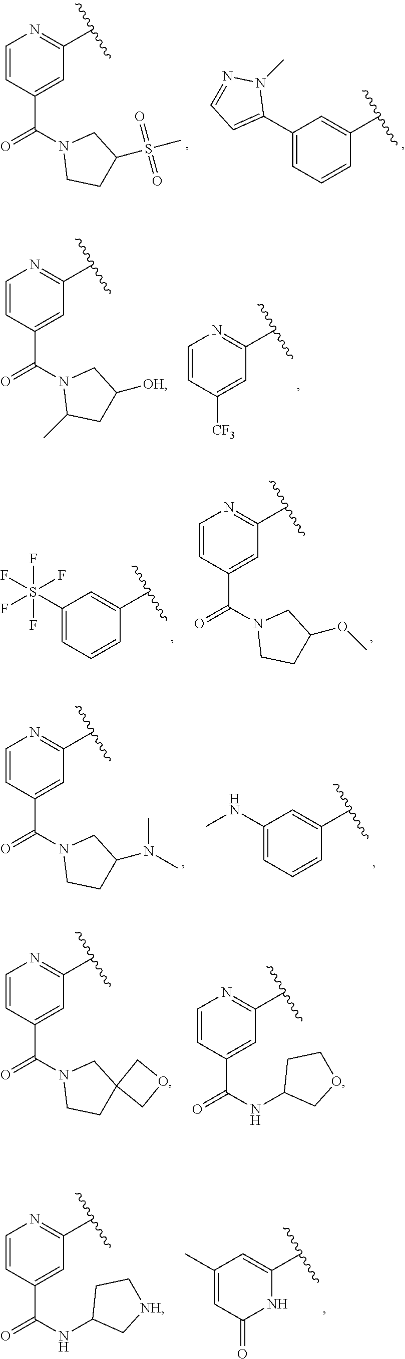

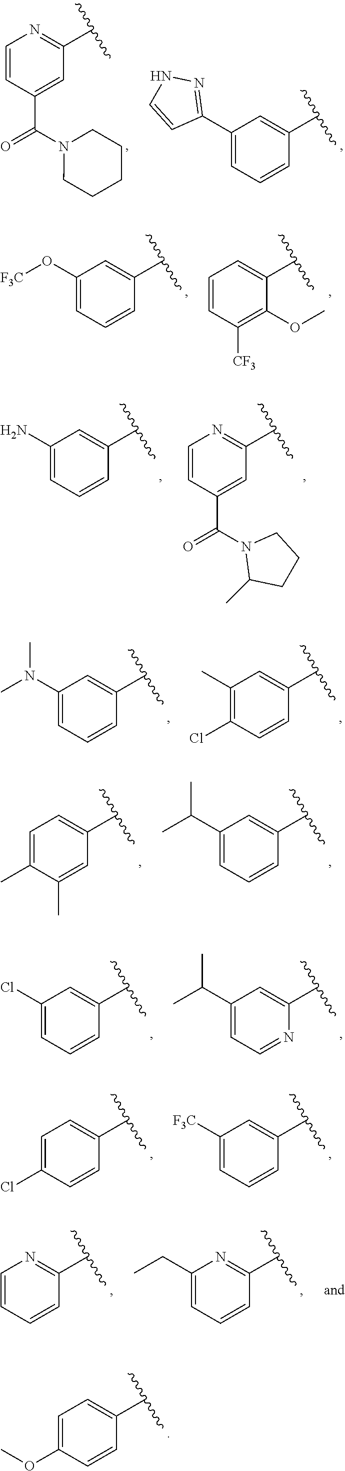

81. The compound or tautomer thereof, or pharmaceutically acceptable salt of the compound or tautomer, of any one of claims 68-72, wherein 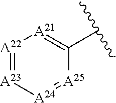
is selected from the group consisting of

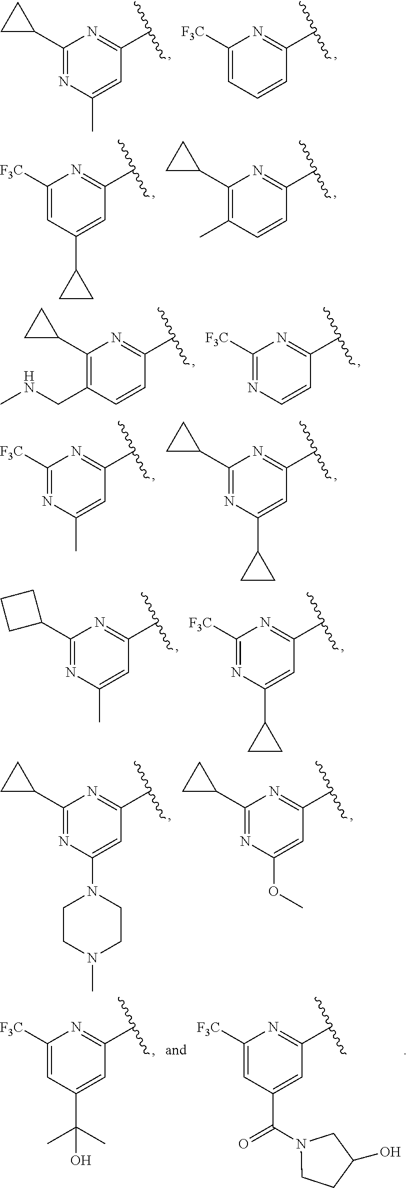

82. The compound or tautomer thereof, or pharmaceutically acceptable salt of the compound or tautomer, of any one of claims 68-72, wherein 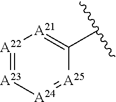
is selected from the group consisting of

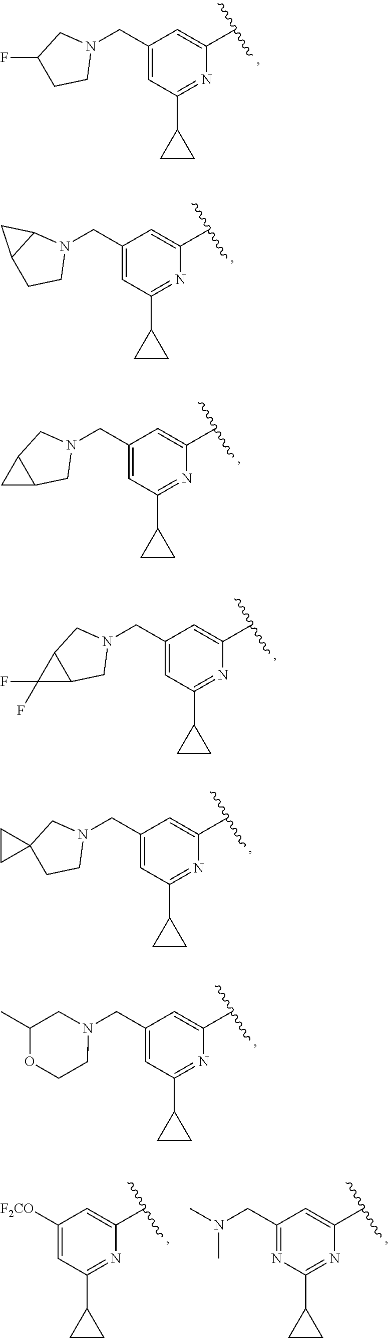

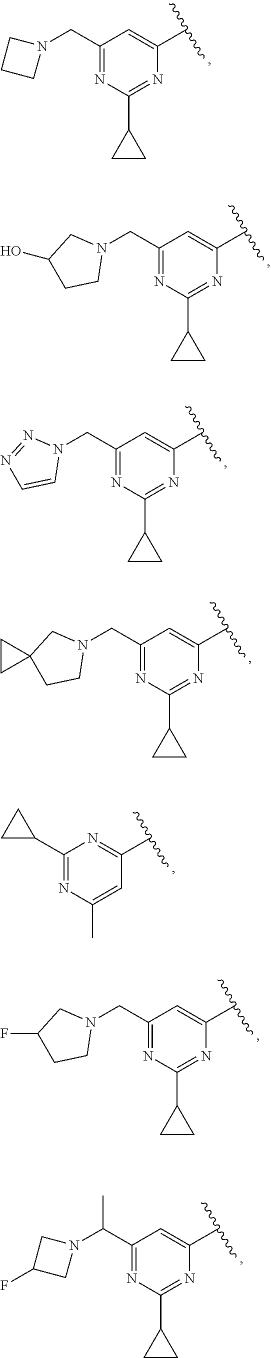

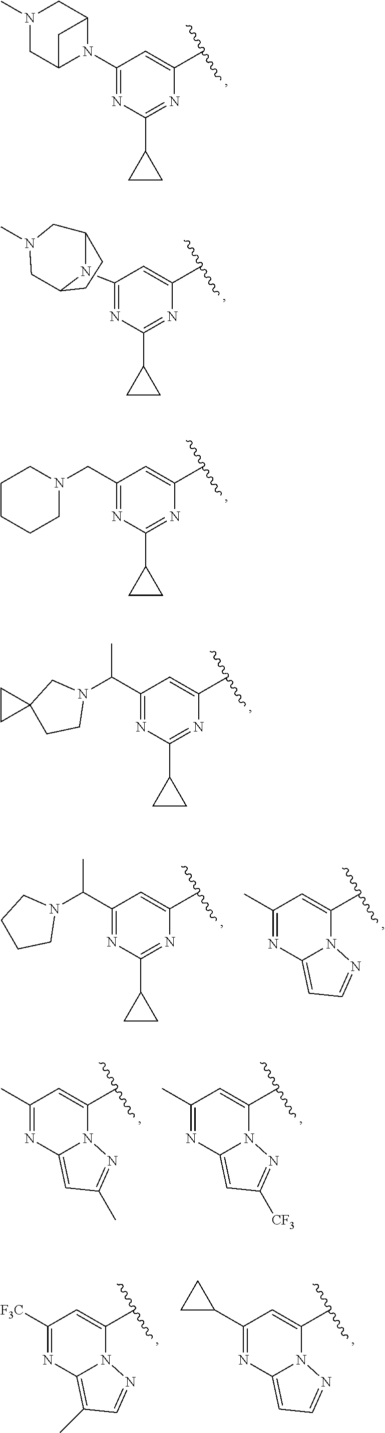

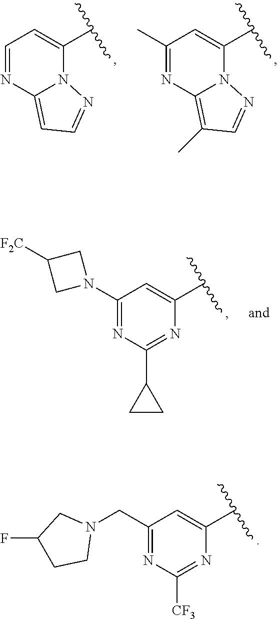

83. The compound or tautomer thereof, or pharmaceutically acceptable salt of the compound or tautomer, of any one of claims 68-82, wherein each K2 is independently selected from the group consisting of:
F, Cl, Br, I, —CN,
C₁-C₈ alkyl optionally substituted with —OH or —O—C₁-C₈ alkyl,
C₃-C₈ cycloalkyl optionally substituted with —OH or —O—C₁-C₈ alkyl,
—O—C₁-C₈ alkyl,
—O—C₁-C₈ haloalkyl, and
—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl.

84. The compound or tautomer thereof, or pharmaceutically acceptable salt of the compound or tautomer, of any one of claims 68-82, wherein each K2 is independently
C₃-C₈ cycloalkyl optionally substituted with —C₁-C₈ alkyl, —OH or —O—C₁-C₈ alkyl, or
—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, Ci-C's hydroxyalkyl, —CN, or —O—C₁-C₈ alkyl.

85. The compound or tautomer thereof, or pharmaceutically acceptable salt of the compound or tautomer, of any one of claims 68-82, wherein two vicinal K2 groups are taken together with the atoms to which they are attached to form a three- to six-membered carbocyclic or heterocyclic ring, a phenyl ring, or a five- to six-membered heteroaryl ring, wherein the ring formed by the two vicinal K2 groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

86. The compound or tautomer thereof, or pharmaceutically acceptable salt of the compound or tautomer, of any one of claims 68-82, wherein two vicinal K2 groups are taken together with the atoms to which they are attached to form a five-membered carbocyclic or heterocyclic ring, wherein the five-membered carbocyclic or heterocyclic ring are each optionally substituted with one or two substituents selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

87. The compound or tautomer thereof, or pharmaceutically acceptable salt of the compound or tautomer, of any one of claims 68-84, wherein m2 is 0.

88. The compound or tautomer thereof, or pharmaceutically acceptable salt of the compound or tautomer, of any one of claims 68-84, wherein m2 is 1.

89. The compound or tautomer thereof, or pharmaceutically acceptable salt of the compound or tautomer, of any one of claims 68-88, wherein R²⁶ is H.

90. The compound or tautomer thereof, or pharmaceutically acceptable salt of the compound or tautomer, of any one of claims 68-76 or 83-88, wherein A₂₅ is CR²⁵, and R²⁵, R²⁶ and the intervening atoms are taken together to form a five-membered lactam ring, such that the fragment 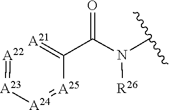
is

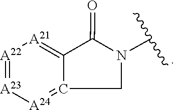

91. The compound or tautomer thereof, or pharmaceutically acceptable salt of the compound or tautomer, of claim 90, wherein at least one of R²¹, R²², R²³, and R²⁴ is selected from the group consisting of: a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₃-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

92. The compound or tautomer thereof, or pharmaceutically acceptable salt of the compound or tautomer, of claim 90, wherein R²² is a —(C₁-C₄ alkylene)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

93. The compound or tautomer thereof, or pharmaceutically acceptable salt of the compound or tautomer, of claim 90, wherein R²² is a —(C₁-C₂ alkylene)-(four-membered heterocyclic ring), a —(C₁-C₂ alkylene)-(five-membered heterocyclic ring), or a —(C₁-C₂ alkylene)-(six-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

94. The compound or tautomer thereof, or pharmaceutically acceptable salt of the compound or tautomer, of claim 90, wherein R²² is a —CH₂-(four- to eight-membered heterocyclic ring) or a —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

95. The compound or tautomer thereof, or pharmaceutically acceptable salt of the compound or tautomer, of claim 90, wherein R²² is —CH₂-(pyrrolidinyl) or —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

96. The compound or tautomer thereof, or pharmaceutically acceptable salt of the compound or tautomer, of claim 90, wherein R²² is —CH₂-(azetidinyl) or —CH(CH₃)-azetidinyl, wherein the azetidinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

97. The compound or tautomer thereof, or pharmaceutically acceptable salt of the compound or tautomer, of any one of claims 90-96, wherein R²⁴ is selected from the group consisting of H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl.

98. The compound or tautomer thereof, or pharmaceutically acceptable salt of the compound or tautomer, of any one of claims 90-96, wherein R²⁴ is CF₃.

99. The compound or tautomer thereof, or pharmaceutically acceptable salt of the compound or tautomer, of claim 90, wherein 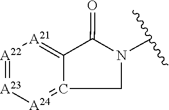
is selected from the group consisting of

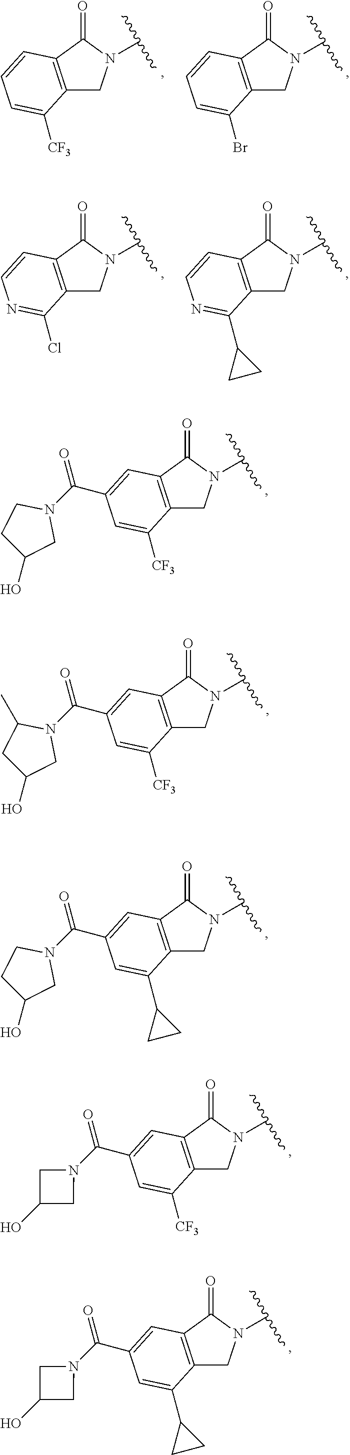

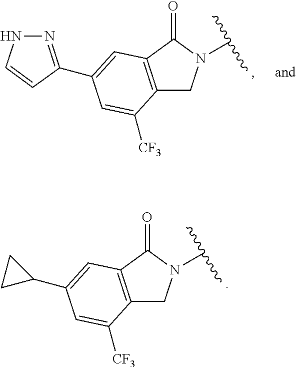 [SMILES: *N1Cc2c(cc(-c3cc[nH]n3)cc2C(F)(F)F)C1=O ; *N1Cc2c(cc(C3CC3)cc2C(F)(F)F)C1=O]

100. The compound or tautomer thereof, or pharmaceutically acceptable salt of the compound or tautomer, of claim 90, wherein 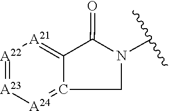
is selected from the group consisting of

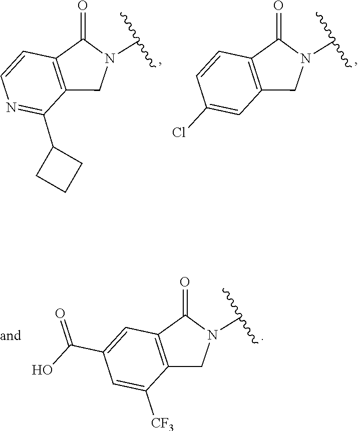 [SMILES: *N1Cc2c(ccnc2C2CCC2)C1=O ; *N1Cc2cc(Cl)ccc2C1=O ; *N1Cc2c(cc(C(=O)O)cc2C(F)(F)F)C1=O]

101. The compound or tautomer thereof, or pharmaceutically acceptable salt of the compound or tautomer, of claim 90, wherein 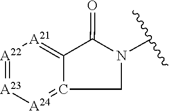
is selected from the group consisting of

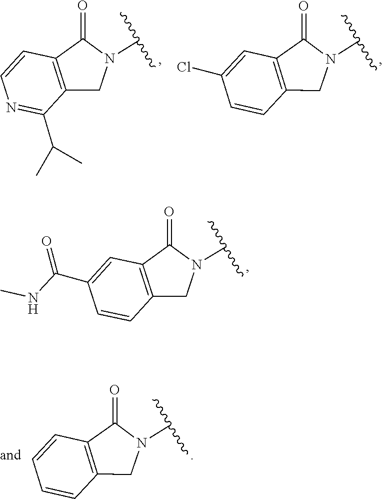 [SMILES: *N1Cc2c(ccnc2C(C)C)C1=O ; *N1Cc2ccc(Cl)cc2C1=O ; *N1Cc2ccc(C(=O)NC)cc2C1=O ; *N1Cc2ccccc2C1=O]

102. The compound or tautomer thereof, or pharmaceutically acceptable salt of the compound or tautomer, of any one of claims 68-101, wherein R²⁷ is methyl.

103. The compound or tautomer thereof, or pharmaceutically acceptable salt of the compound or tautomer, of any one of claims 68-101, wherein R²⁷ is H, F, CF₃ or —CH₂OCH₃.

104. The compound or tautomer thereof, or pharmaceutically acceptable salt of the compound or tautomer, of any one of claims 68-101, wherein R²⁸ and R²⁹ are taken together with the atoms to which they are attached to form a five- or six-membered cycloalkyl or heterocyclyl ring, wherein the three- to six-membered cycloalkyl or heterocyclyl ring are each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

105. The compound or tautomer thereof, or pharmaceutically acceptable salt of the compound or tautomer, of any one of claims 68-103, wherein R²⁸ and R²⁹ are taken together to form a cyclopropyl, cyclobutyl, cyclopentyl, cyclohexyl, aziridinyl, oxiranyl, thiiranyl, azetidinyl, oxetanyl, thietanyl, pyrrolidinyl, pyrazolidinyl, imidazolidinyl, tetrahydrofuranyl, 1,3-dioxolanyl, tetrahydrothiophenyl, oxathiolanyl, sulfolanyl, piperidinyl, piperazinyl, tetrahydropyranyl, dioxanyl, thianyl, dithianyl, trithianyl, morpholinyl, or thiomorpholinyl ring.

106. The compound or tautomer thereof, or pharmaceutically acceptable salt of the compound or tautomer, of any one of claims 68-105, wherein R³⁰ is selected from the group consisting of H, F, Cl, Br, I, and C₁-C₈ alkyl.

107. The compound or tautomer thereof, or pharmaceutically acceptable salt of the compound or tautomer, of claim 106, wherein R³⁰ is H.

108. The compound or tautomer thereof, or pharmaceutically acceptable salt of the compound or tautomer, of any one of claims 68-107, wherein the absolute configuration of the carbon atom to which R²⁷ and R²⁸ are attached is (R).

109. The compound or tautomer thereof, or pharmaceutically acceptable salt of the compound or tautomer, of any one of claims 68-107, wherein the absolute configuration of the carbon atom to which R²⁷ and R²⁸ are attached is (S).

110. The compound or tautomer thereof, or pharmaceutically acceptable salt of the compound or tautomer, of claim 68, wherein [symbol] is a double bond, Y3 is C and Y4 is C.

111. The compound or tautomer thereof, or pharmaceutically acceptable salt of the compound or tautomer, of any one of claims 68-109, wherein Ring B2 is a five-membered heteroaryl ring containing at least one N, O, or S ring atom, wherein Ring B2 is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

112. The compound or tautomer thereof, or pharmaceutically acceptable salt of the compound or tautomer, of any one of claims 68-109, wherein Ring B2 is a five-membered heteroaryl ring containing at least one N, O, or S ring atom, wherein Ring B2 is optionally substituted with one, two, or three substituents independently selected from the group consisting of F, Cl, Br, and I.

113. The compound or tautomer thereof, or pharmaceutically acceptable salt of the compound or tautomer, of any one of claims 68-109, wherein Ring B2 is a five-membered heteroaryl ring containing at least one N, O, or S ring atom, wherein Ring B2 is optionally substituted with one, two, or three substituents independently selected from the group consisting of methyl, ethyl, cyclopropyl, and —CH₂OH.

114. The compound or tautomer thereof, or pharmaceutically acceptable salt of the compound or tautomer, of any one of claims 68-109, wherein Ring B2 is selected from the group consisting of pyrrole, imidazole, 1,2,4-triazole, 1,2,3-triazole, pyrazole, tetrazole, oxadiazole, oxazole, and isoxazole, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

115. The compound or tautomer thereof, or pharmaceutically acceptable salt of the compound or tautomer, of any one of claims 68-109, wherein Ring B2 is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

116. The compound or tautomer thereof, or pharmaceutically acceptable salt of the compound or tautomer, of any one of claims 68-109, wherein Ring B2 is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with methyl, ethyl, cyclopropyl, or —CH₂OH.

117. The compound or tautomer thereof, or pharmaceutically acceptable salt of the compound or tautomer, of any one of claims 68-109, wherein Ring B2 is selected from the group consisting of

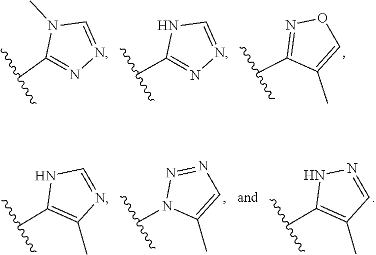

118. The compound or tautomer thereof, or pharmaceutically acceptable salt of the compound or tautomer, of any one of claims 68-109, wherein Ring B2 is selected from the group consisting of

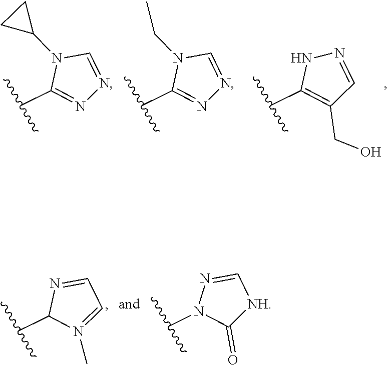

119. The compound or tautomer thereof, or pharmaceutically acceptable salt of the compound or tautomer, of any one of claims 68-109, wherein Ring B2 is selected from the group consisting of

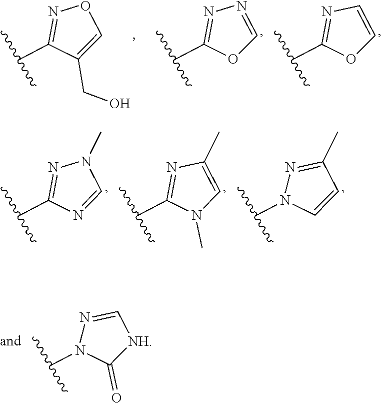

120. The compound or tautomer thereof, or pharmaceutically acceptable salt of the compound or tautomer, of any one of claims 68-109, wherein Ring B2 is selected from the group consisting of

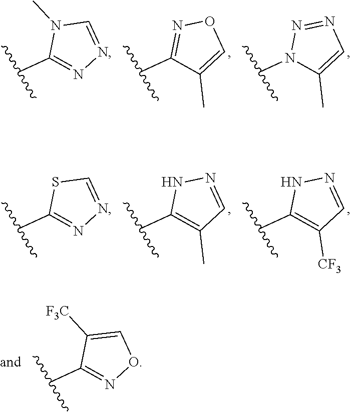

121. The compound or tautomer thereof, or pharmaceutically acceptable salt of the PGP-1990 CI compound or tautomer, of any one of claims 68-109, wherein Ring B2 is 4-methyl-4H-1,2,4-triazol-3-yl.

122. The compound or tautomer thereof, or pharmaceutically acceptable salt of the compound or tautomer, of claim 68, selected from the group consisting of Compounds 282, 283, and 283b of Table 1, including “a” and “b” variants of the compounds, tautomers thereof, and pharmaceutically acceptable salts of the compounds or tautomers.

123. The compound or tautomer thereof, or pharmaceutically acceptable salt of the compound or tautomer, of claim 68, which is Compound 282, a tautomer thereof, or pharmaceutically acceptable salt of the compound or tautomer.

124. A pharmaceutical composition comprising a Cbl-b inhibitor of Formula (III-A): 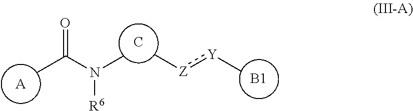
or a tautomer thereof, or a pharmaceutically acceptable salt of any of the foregoing, and a pharmaceutically acceptable excipient;
wherein Ring A, 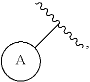
is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;
wherein Ring A is attached to the adjacent carbonyl carbon via a carbon atom of Ring A;
each R^{A} is independently selected from the group consisting of:
H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,
—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five-to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring,
(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three-to eight-membered heterocyclic ring where the carbocyclic or heterocyclic ring is optionally substituted with C₁-C₄ alkyl or OH;
—S(═O)₂—C₁-C₈ alkyl,
—SF₅, and
—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five-to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
R⁶ is H, or
R⁶ and the amide group to which R⁶ is connected attach to Ring A to form a five-membered lactam ring fused to Ring A, such that the fragment 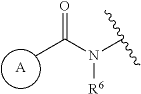
is 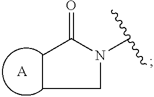
Ring C, 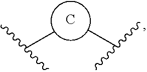
is selected from the group consisting of: 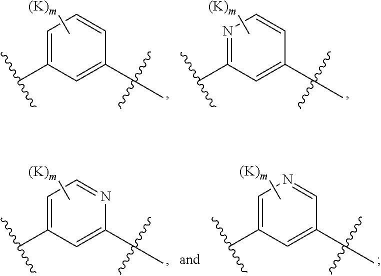
each K is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —C₁-C₈ alkyl, —OH or —O—C₁-C₈ alkyl,
—O—C₁-C₈ alkyl optionally substituted with —OH,
—O—C₁-C₈ haloalkyl,
—O-(three- to six-membered heterocyclic ring) optionally substituted with C₁-C₈ alkyl,
a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;
m is 0, 1, or 2; [symbol]
is a single bond or a double bond,
wherein when [symbol] is a single bond,
Y is C(R⁹)(R¹⁰), S, or O; and
Z is C(R⁷)(R⁸), and
when [symbol] is a double bond,
Y is C(R⁹); and
Z is C(R⁸),
R⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,
R⁸ is selected from the group consisting of H, F, —OH, C₁-C₈ alkyl optionally substituted with —OH or —O—C₁-C₈ alkyl, C₂-C₈ alkenyl, C₁-C₈ haloalkyl, and —O—C₁-C₈ alkyl optionally substituted with —OH or —O—C₁-C₈ alkyl, or
when Y is C(R⁹)(R¹⁰) or C(R⁹), R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl, or
R⁷ and R⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;
R⁹ and R¹⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or
R⁹ is taken together with R⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; or
R⁹ and R¹⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and
Ring B, 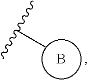
is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

125. The pharmaceutical composition of claim 124, wherein the compound of Formula (III-A) is a compound of Formula (III): 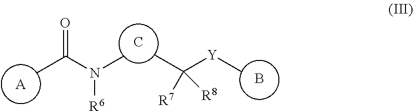
or a tautomer thereof, or a pharmaceutically acceptable salt of any of the foregoing, and a pharmaceutically acceptable excipient;
wherein Ring A, 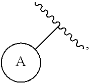
is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;
wherein Ring A is attached to the adjacent carbonyl carbon via a carbon atom of Ring A;
each R^{A} is independently selected from the group consisting of:
H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂,
—C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,
—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five-to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring,
—(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three-to eight-membered heterocyclic ring where the carbocyclic or heterocyclic ring is optionally substituted with C₁-C₄ alkyl or OH;
—S(═O)₂—C₁-C₈ alkyl,
—SF₅, and
—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five-to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
R⁶ is H, or
R⁶ and the amide group to which R⁶ is connected attach to Ring A to form a five-membered lactam ring fused to Ring A, such that the fragment 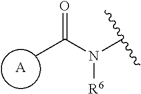
is 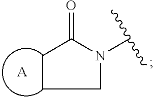
Ring C, 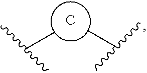
is selected from the group consisting of: 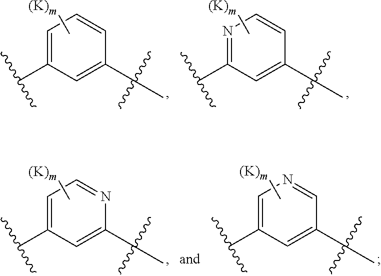
each K is independently selected from the group consisting of:
, Cl, Br, I, —CN, —OH,
—C₁-C₈ alkyl optionally substituted with —OH or —O—C₁-C₈ alkyl,
—C₃-C₈ cycloalkyl optionally substituted with —OH or —O—C₁-C₈ alkyl,
—O—C₁-C₈ alkyl optionally substituted with —OH,
—O—C₁-C₈ haloalkyl,
—O-(three- to six-membered heterocyclic ring) optionally substituted with C₁-C₈ alkyl,
a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, Cr C₈ alkyl optionally substituted with —OH, C₃-C₈ cycloalkyl optionally substituted with —OH or C₁-C₄ alkyl, and four- to eight-membered heterocyclyl optionally substituted with —OH or C₁-C₄ alkyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;
m is 0, 1, or 2;
R⁷ is selected from the group consisting of H, F, —OH, C₁-C₈ alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,
R⁸ is selected from the group consisting of H, F, —OH, C₁-C₈ alkyl optionally substituted with —OH or —O—C₁-C₈ alkyl, C₂-C₈ alkenyl, C₁-C₈ haloalkyl, and —O—C₁-C₈ alkyl optionally substituted with —OH or —O—C₁-C₈ alkyl, or
when Y is C(R⁹)(R¹⁰), R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl, or
R⁷ and R⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;
Y is C(R⁹)(R¹⁰) or S;
R⁹ and R¹⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or
R⁹ is taken together with R⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; or
R⁹ and R¹⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and
Ring B, 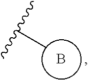
is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

126. The pharmaceutical composition of claim 124 or claim 125, wherein
Ring A, 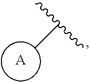
is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;
wherein Ring A is attached to the adjacent carbonyl carbon via a carbon atom of Ring A;
each R^{A} is independently selected from the group consisting of:
H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—Cr C₈ haloalkyl, F, Cl, Br, I, —CN, and —NR^{B}R^{C}, where R^{B} and R^{c} are independently H, C₁-C₈ alkyl, or C₁-C₈ haloalkyl,
—NR^{a}R^{b} where R^{a} and R^{b} are independently H, C₁-C₈ alkyl, C₁-C₈ haloalkyl, or C₃-C₈ cycloalkyl, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five- to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to eight-membered heterocyclic ring, a five- to eight-membered heteroaryl ring, a —(C₁-C₄ alkylene)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, or I,
(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
—S(═O)₂—C₁-C₈ alkyl,
—SF₅, and
—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
R⁶ is H, or
R⁶ and the amide group to which R⁶ is connected attach to Ring A to form a five-membered lactam ring fused to Ring A, such that the fragment 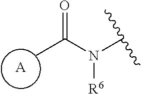
is 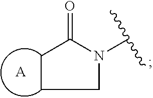
Ring C, 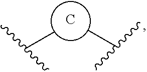
is selected from the group consisting of: 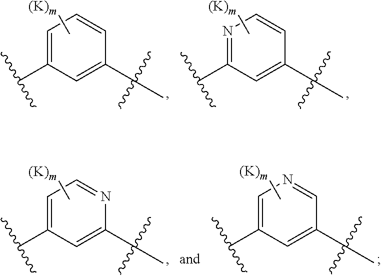
each K is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —OH or —O—C₁-C₈ alkyl,
—O—C₁-C₈ alkyl,
—O—C₁-C₈ haloalkyl,
a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;
m is 0, 1, or 2;
R⁷ is selected from the group consisting of H, F, —OH, Ci-C's alkyl optionally substituted with —OH or —O—C₁-C₈ alkyl, C₂-C₈ alkenyl, C₁-C₈ haloalkyl, —O—C₁-C₈ haloalkyl, and —O—C₁-C₈ alkyl optionally substituted with —OH or —O—C₁-C₈ alkyl,
R⁸ is selected from the group consisting of H, F, —OH, Ci-C's alkyl optionally substituted with —OH or —O—C₁-C₈ alkyl, C₂-C₈ alkenyl, C₁-C₈ haloalkyl, and —O—C₁-C₈ alkyl optionally substituted with —OH or —O—C₁-C₈ alkyl, or
when Y is C(R⁹)(R¹⁰), R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl, or
R⁷ and R⁸ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl; and
Y is C(R⁹)(R¹⁰) or S;
R⁹ and R¹⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl, or
R⁹ is taken together with R⁸ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl; and
Ring B, 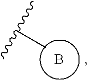
is a five-membered heteroaryl ring containing at least one N, O, or S ring atom, wherein Ring B is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

127. The pharmaceutical composition of any one of claims 124-126, wherein ring A is 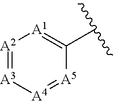
wherein
A¹ is CR¹ or N,
A² is CR² or N,
A³ is CR³ or N,
A⁴ is CR⁴ or N, and
A⁵ is CR⁵ or N,
wherein no more than two of A¹, A², A³, A⁴, and A⁵ are N; and
R¹, R², R³, R⁴, and R⁵ are each independently selected from R^{A}.

128. The pharmaceutical composition of claim 127, wherein at least one of R¹, R², R³, and R⁴ is selected from the group consisting of: a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

129. The pharmaceutical composition of claim 127, wherein at least one of R¹, R², R³, and R⁴ is selected from the group consisting of: —C₁-C₈ haloalkyl-OH, —C₁-C₈ haloalkyl-COOH, —CO(C₁-C₈ alkyl), a nine-membered heterocyclic ring, —CH(C₁-C₈ alkyl) (four-to eight-membered heterocyclic ring), a —CH(C₁-C₈ haloalkyl)-(four- to eight-membered heterocyclic ring), a —CH(OH)-(C₆-C₁₄ aryl ring), and a —O-(four- to eight-membered heterocyclic ring), wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring.

130. The pharmaceutical composition of claim 127 or claim 128, wherein R² is a —(C₁-C₄ alkylene)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

131. The pharmaceutical composition of claim 127 or claim 128, wherein R² is a —(C₁-C₂ alkylene)-(four-membered heterocyclic ring), a —(C₁-C₂ alkylene)-(five-membered heterocyclic ring), or a —(C₁-C₂ alkylene)-(six-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

132. The pharmaceutical composition of claim 127 or claim 128, wherein R² is a —CH₂-(four- to eight-membered heterocyclic ring) or a —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

133. The pharmaceutical composition of claim 127 or claim 128, wherein R² is —CH₂-(pyrrolidinyl) or —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

134. The pharmaceutical composition of claim 127 or claim 128, wherein R² is —CH₂-(azetidinyl) or —CH(CH₃)-azetidinyl, wherein the azetidinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

135. The pharmaceutical composition of any one of claims 127-134, wherein R⁴ is selected from the group consisting of H, F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl.

136. The pharmaceutical composition of any one of claims 127-134, wherein R⁴ is CF₃.

137. The pharmaceutical composition of any one of claims 124-126, wherein ring A is selected from a five- to ten-membered heterocyclic ring system optionally substituted with one, two, or three independently chosen R^{A} groups or a five- to ten-membered heteroaryl ring system optionally substituted with one, two, or three independently chosen R^{A} groups.

138. The pharmaceutical composition of any one of claims 124-126, wherein ring A is selected from the group consisting of pyridyl, quinolinyl, isoquinolinyl, quinoxalinyl, cinnolinyl, quinazolinyl, naphthyridinyl, benzoxazolyl, benzothiazolyl, benzoimidazoyl, pyrrolyl, pyrazolyl, imidazolyl, triazolyl, tetrazolyl, furanyl, isoxazolyl, oxazolyl, oxadiazolyl, thiophenyl, isothiazolyl, thiazolyl, thiadiazolyl, pyridazinyl, pyrimidinyl, pyrazinyl, triazinyl, tetrazinyl, indolyl, isoindolyl, indazolyl, benzotriazolyl, benzofuranyl, benzoisoxazolyl, benzoxadiazolyl, benzothiophenyl, benzoisothiazolyl, benzothiadiazolyl, pyrrolopyridinyl, pyrazolopyridinyl, imidazopyridinyl, triazolopyridinyl, furopyridinyl, oxazolopyridinyl, isoxazolopyridinyl, oxadiazolopyridinyl, thienopyridinyl, thiazolopyridinyl, isothiazolopyridinyl, thiadiazolopyridinyl, thienopyridinyl, phthalazinyl, pyrazolothiazolyl, pyrazolothiazolyl imidazothiazolyl, tetrahydroquinolinyl, tetrahydroisoquinolinyl, decahydroquinolinyl, decahydroisoquinolinyl, indolinyl, isoindolinyl, tetrahydronaphthyridinyl and hexahydrobenzoimidazolyl, each optionally substituted with one, two, or three independently chosen R^{A} groups.

139. The pharmaceutical composition of any one of claims 124-126, wherein ring A is selected from the group consisting of quinolinyl optionally substituted with one, two, or three independently chosen R^{A} groups, isoquinolinyl optionally substituted with one, two, or three independently chosen R^{A} groups, 1H-benzo[d]imidazolyl optionally substituted with one, two, or three independently chosen R^{A} groups, and indolyl optionally substituted with one, two, or three independently chosen R^{A} groups.

140. The pharmaceutical composition of any one of claims 124-126, wherein ring A is selected from the group consisting of 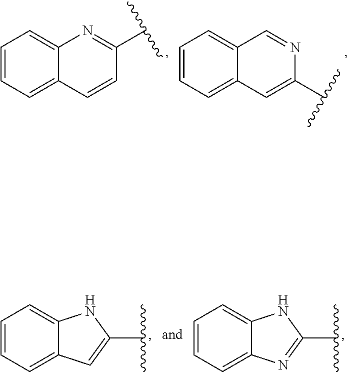
each optionally substituted with one, two, or three independently chosen R^{A} groups.

141. The pharmaceutical composition of any one of claims 124-126, wherein ring A is selected from the group consisting of 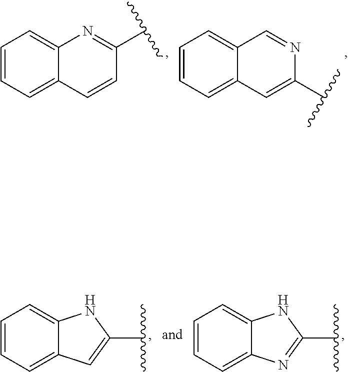
each optionally substituted with one, two, or three substituents independently selected from the group consisting of
F, Cl, Br, I, —CN, C₁-C₈ alkyl, —O—C₁-C₈ alkyl, C₁-C₈ haloalkyl, —O—C₁-C₈ alkyl-heteroaryl,
a three- to eight-membered heterocyclic ring or a five- to eight-membered heteroaryl ring, each heterocyclic ring or heteroaryl ring containing one, two, or three heteroatoms independently selected from the group consisting of O, N, and S and optionally substituted with —OH, oxo, C₃-C₈ alkyl, or —C(═O)—C₁-C₈ alkyl, and
—O—(C₁-C₄ alkylene)-C(═O)NR^{c}R^{d}, wherein R^{c} and R^{d} are independently H or C₁-C₈ alkyl.

142. The pharmaceutical composition of any one of claims 124-126, wherein ring A is selected from the group consisting of 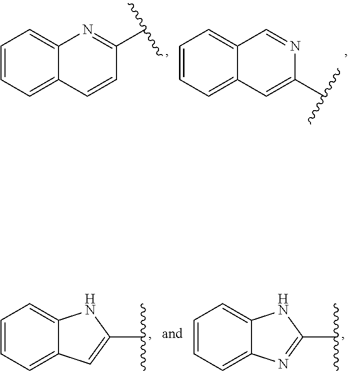
each optionally substituted with one, two, or three substituents independently selected from the group consisting of chloro, fluoro, methyl, isopropyl, cyclopropyl, methoxy, CF₃, CN, propan-2-ol, and —O—CH₂—C(═O)NHCH₃.

143. The pharmaceutical composition of any one of claims 124-126, wherein ring A is selected from the group consisting of pyridine optionally substituted with one, two, or three independently chosen R^{A} groups, pyrimidine optionally substituted with one, two, or three independently chosen R^{A} groups, and pyrazine optionally substituted with one, two, or three independently chosen R^{A} groups.

144. The pharmaceutical composition of any one of claims 124-126, wherein ring A is selected from the group consisting of 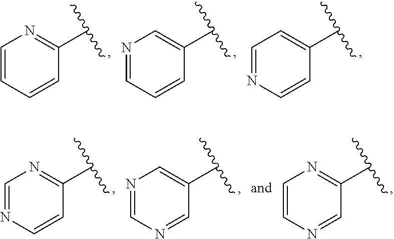
each optionally substituted with one, two, or three independently chosen R^{A} groups.

145. The pharmaceutical composition of any one of claims 124-126, wherein ring A is selected from the group consisting of 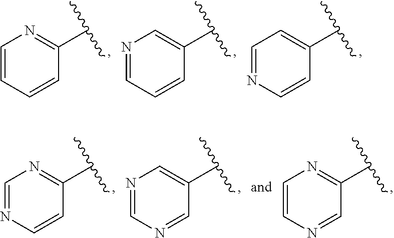
each optionally substituted with one, two, or three groups independently selected from the group consisting of
F, Cl, Br, I, —CN, C₁-C₈ alkyl, —O—C₁-C₈ alkyl, —C₁-C₈ alkyl-OH, C₁-C₈ haloalkyl, C₃-C₈ cycloalkyl, —S(═O)₂—C₁-C₈ alkyl, —(C₁-C₄ alkylene)-NR^{c}R^{d}, and —C(═O)NR^{c}R^{d},
wherein R^{c} and R^{d} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

146. The pharmaceutical composition of any one of claims 124-126, wherein ring A is selected from C₃-C₈ cycloalkyl optionally substituted with one, two, or three independently chosen R^{A} groups, and C₆-C₁₀ aryl optionally substituted with one, two, or three independently chosen R^{A} groups.

147. The pharmaceutical composition of any one of claims 124-126, wherein ring A is selected from the group consisting of cyclopropyl, cyclobutyl, cyclopentyl, cyclohexyl, cycloheptyl, cyclooctyl, phenyl, or naphthyl, each optionally substituted with one, two, or three independently chosen R^{A} groups.

148. The pharmaceutical composition of any one of claims 124-147, wherein ring A is substituted with one, two, or three R^{A} groups selected from the group consisting of: F, Cl, Br, I, —CN, —OH, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, —O—C₁-C₈ alkylene-heterocyclyl-(C₁-C₂ alkylene)-(four-membered heterocyclic ring), a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₃-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

149. The pharmaceutical composition of claim 148, wherein ring A is substituted with an R^{A} group selected from H, F, Cl, Br, I, —CN, —OH, C₁-C₈ alkyl, C₃-C₈ cycloalkyl, —O—C₁-C₈ alkyl, —C₁-C₈ alkyl-OH, and C₁-C₈ haloalkyl.

150. The pharmaceutical composition of claim 148 or claim 149, wherein ring A is substituted with an R^{A} group selected from —CH₂-(four- to eight-membered heterocyclic ring) and —CH(CH₃)-(four- to eight-membered heterocyclic ring), wherein the heterocyclic ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

151. The pharmaceutical composition of claim 148 or claim 149, wherein ring A is substituted with an R^{A} group selected from —CH₂-(pyrrolidinyl) and —CH(CH₃)-pyrrolidinyl, wherein the pyrrolindinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

152. The pharmaceutical composition of claim 148 or claim 149, wherein ring A is substituted with an R^{A} group selected from —CH₂-(azetidinyl) and —CH(CH₃)-azetidinyl, wherein the azetidinyl is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl.

153. The pharmaceutical composition of any one of claims 124-126, wherein ring A is phenyl, optionally substituted with one, two, or three groups independently selected from the group consisting of F, Cl, Br, I, —CN, C₁-C₈ alkyl, —O—C₁-C₈ alkyl, —C₁-C₈ alkyl-OH, C₃-C₈ cycloalkyl, C₁-C₈ haloalkyl, and —C(═O)NR^{c}R^{d}, wherein R^{c} and R^{d} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

154. The pharmaceutical composition of any one of claims 124-126, wherein ring A is selected from the group consisting of

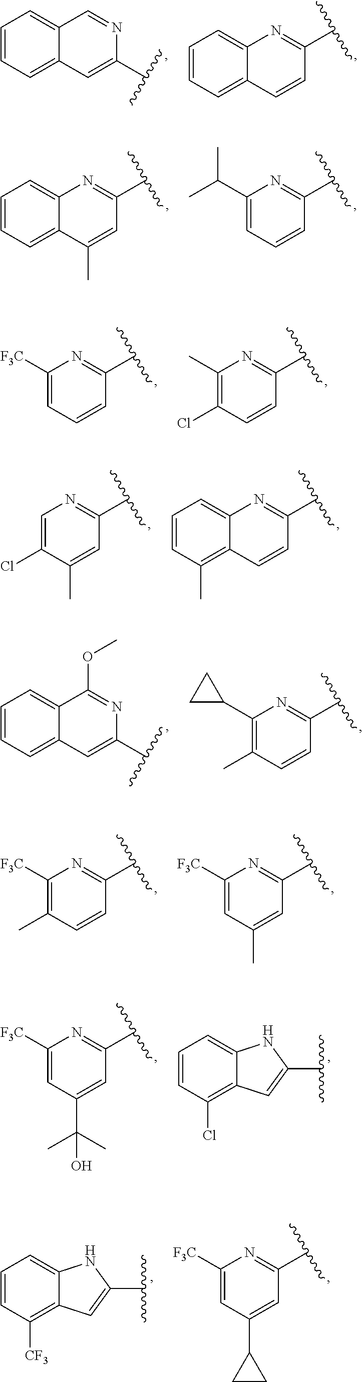

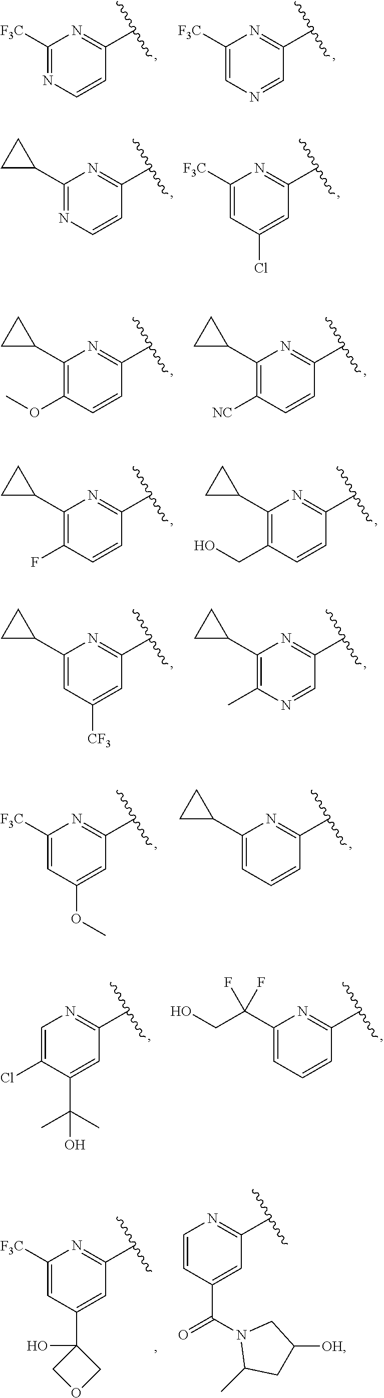

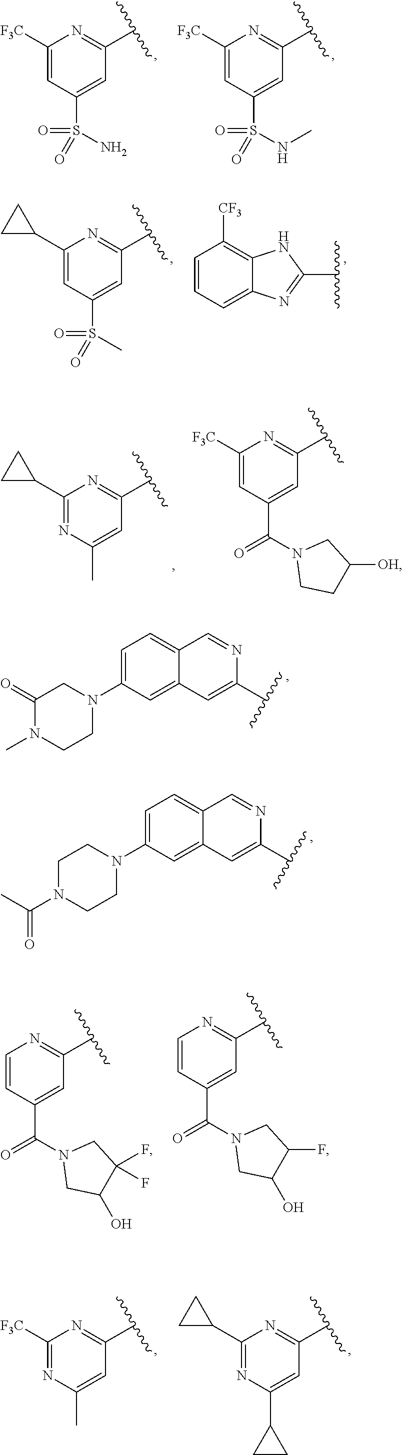

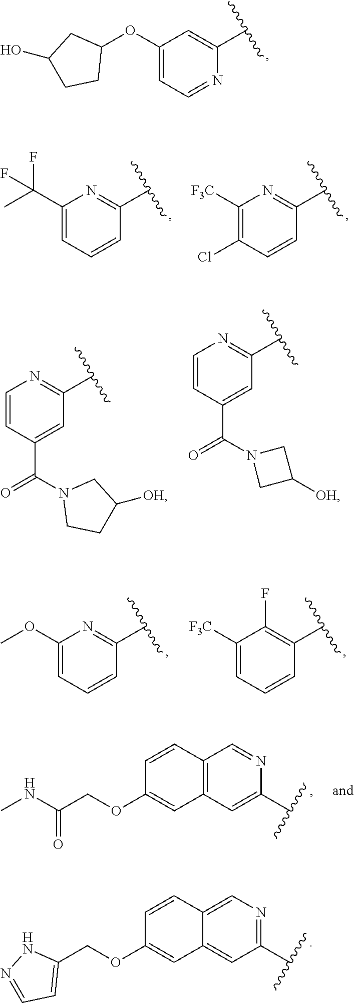

155. The pharmaceutical composition of any one of claims 124-126, wherein ring A is selected from the group consisting of

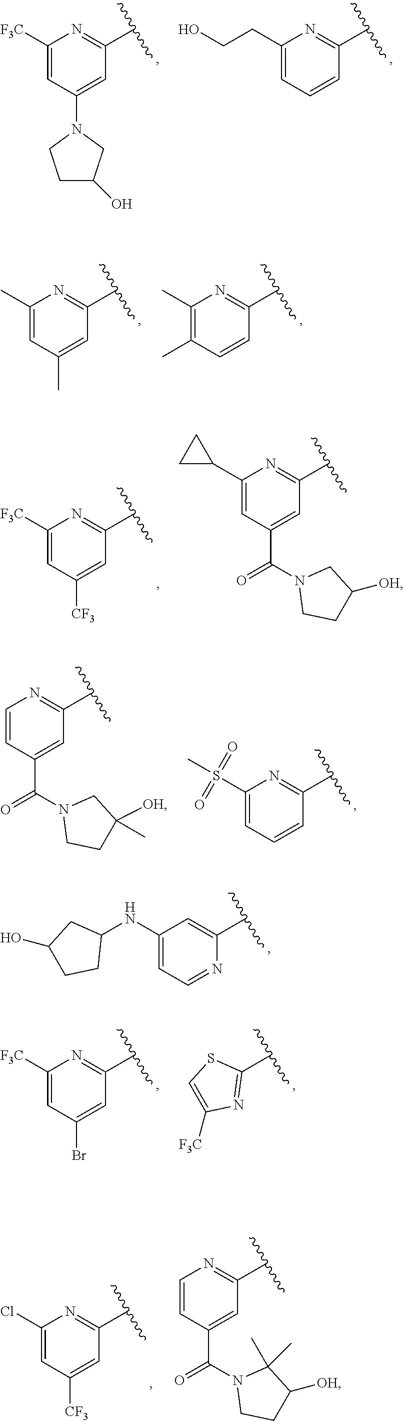

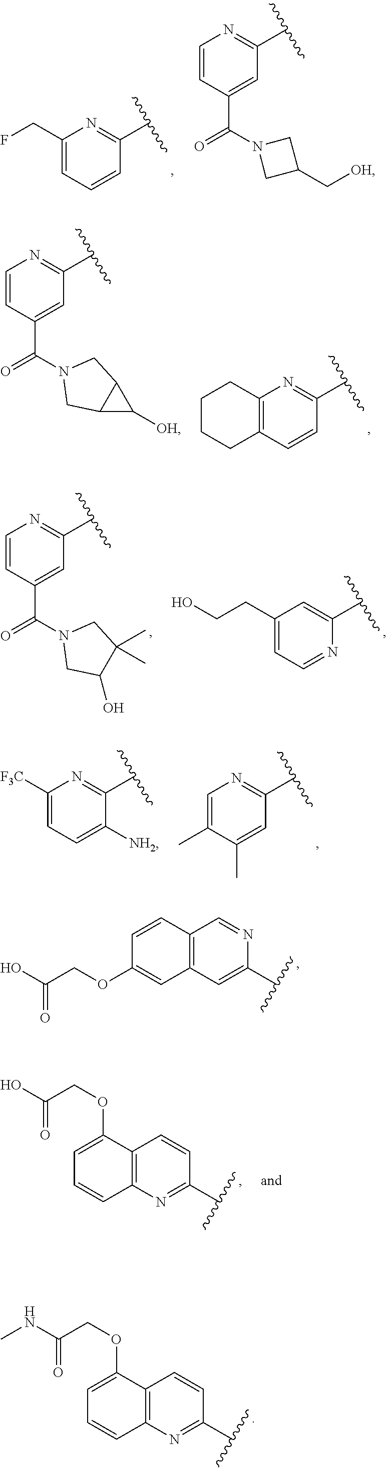

156. The pharmaceutical composition of any one of claims 124-126, wherein ring A is selected from the group consisting of

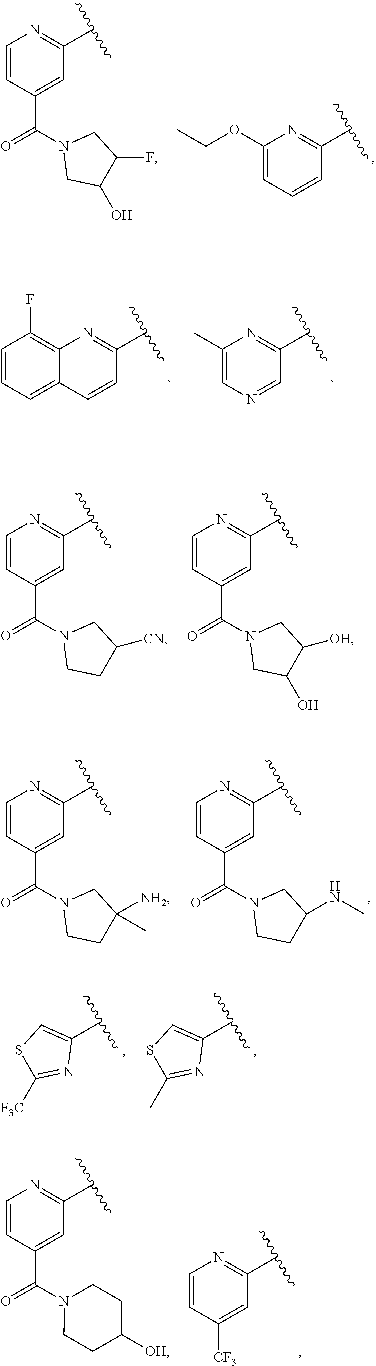

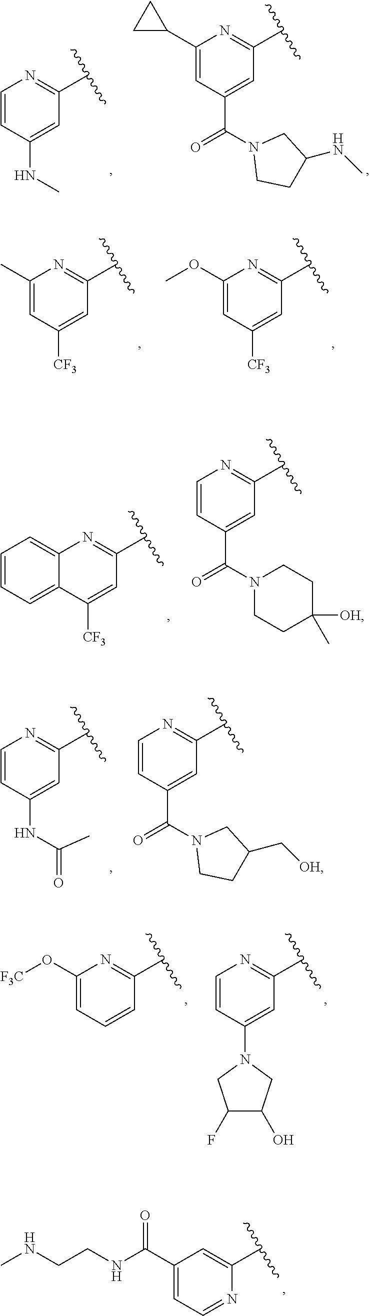

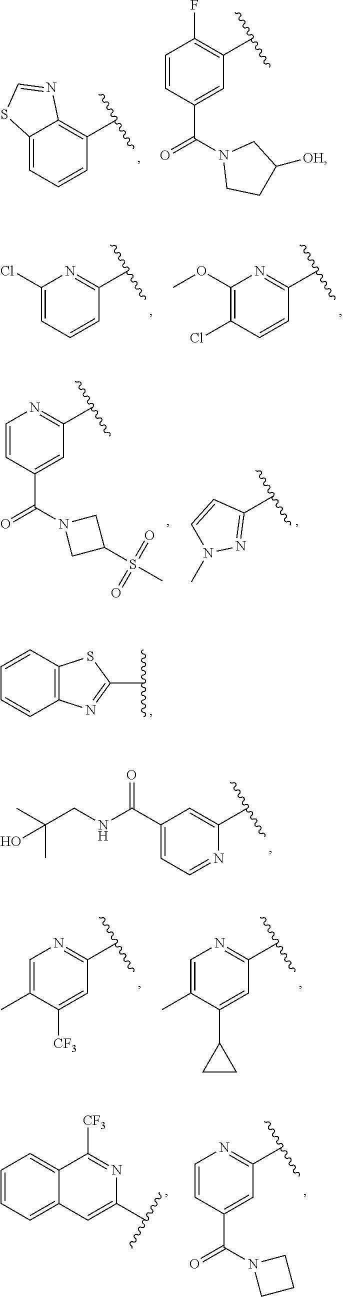

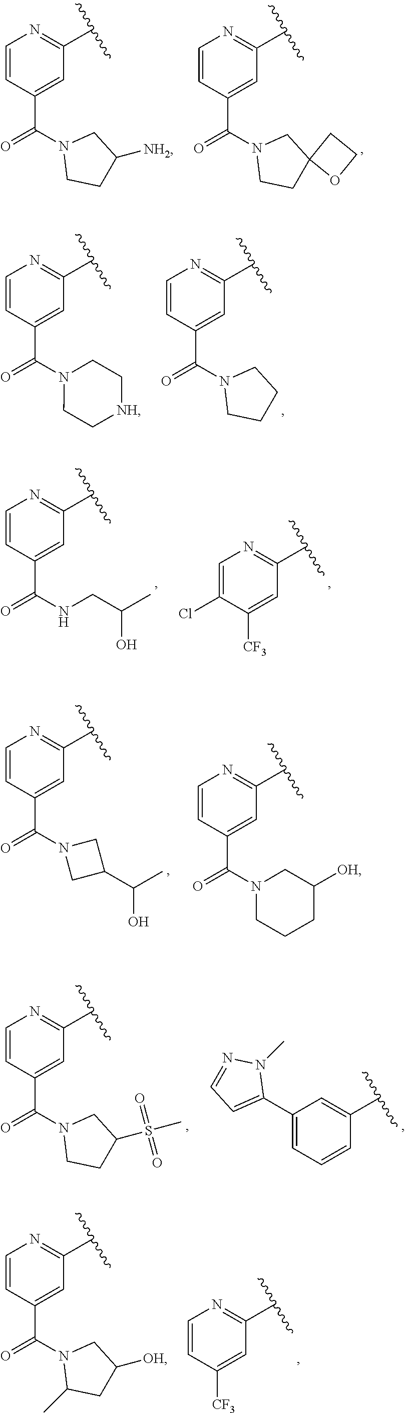

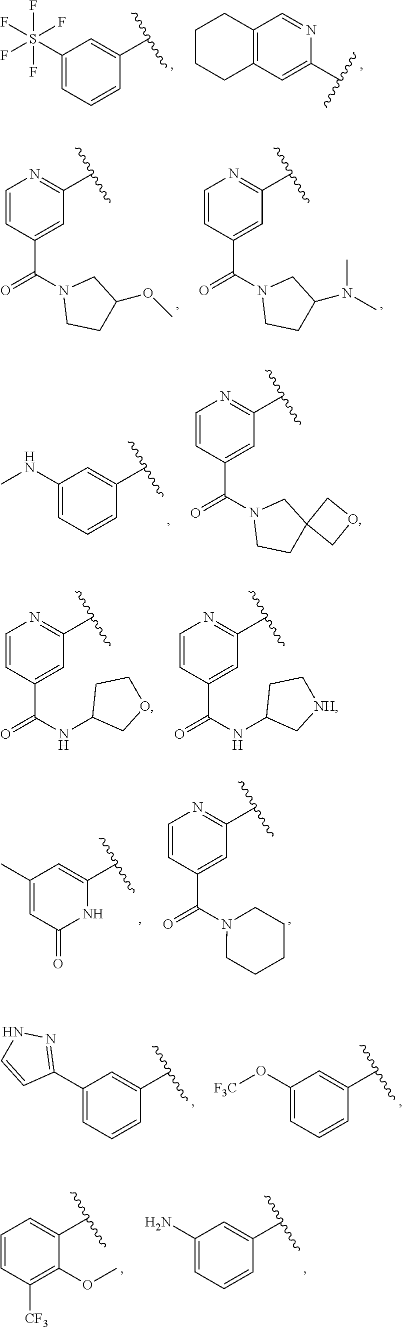

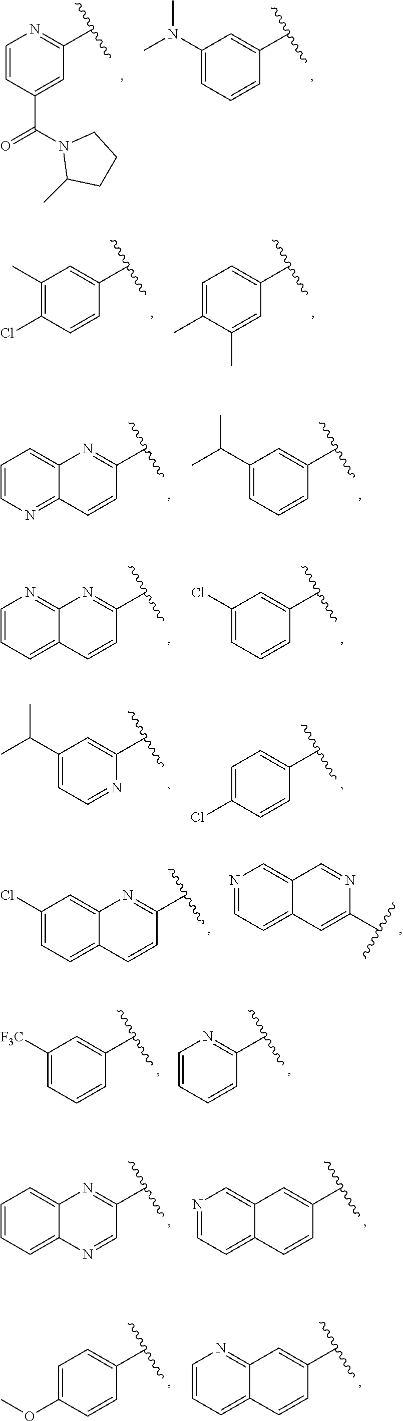

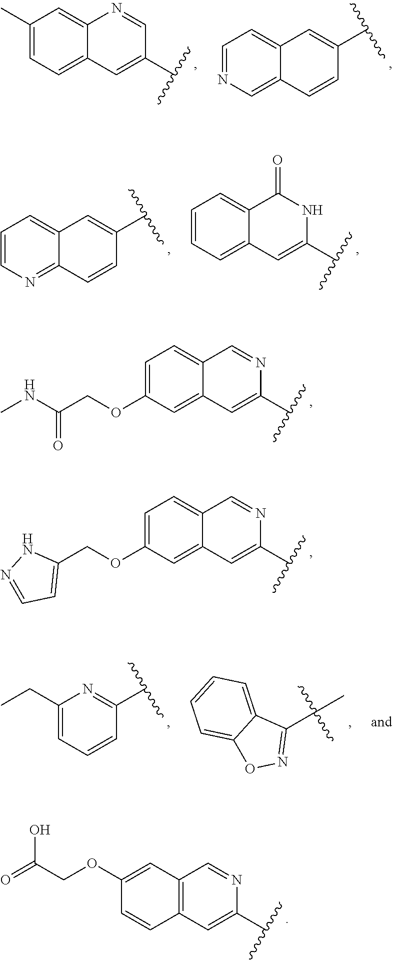

157. The pharmaceutical composition of any one of claims 124-126, wherein ring A is selected from the group consisting of

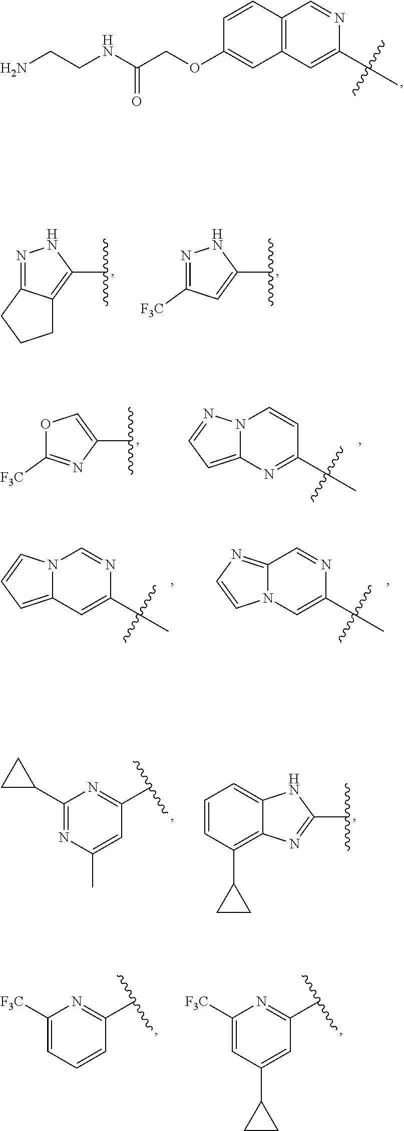

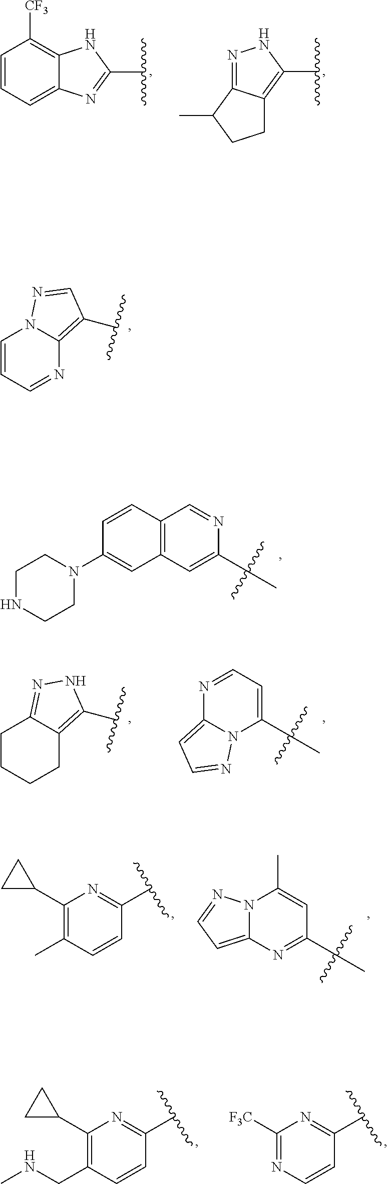

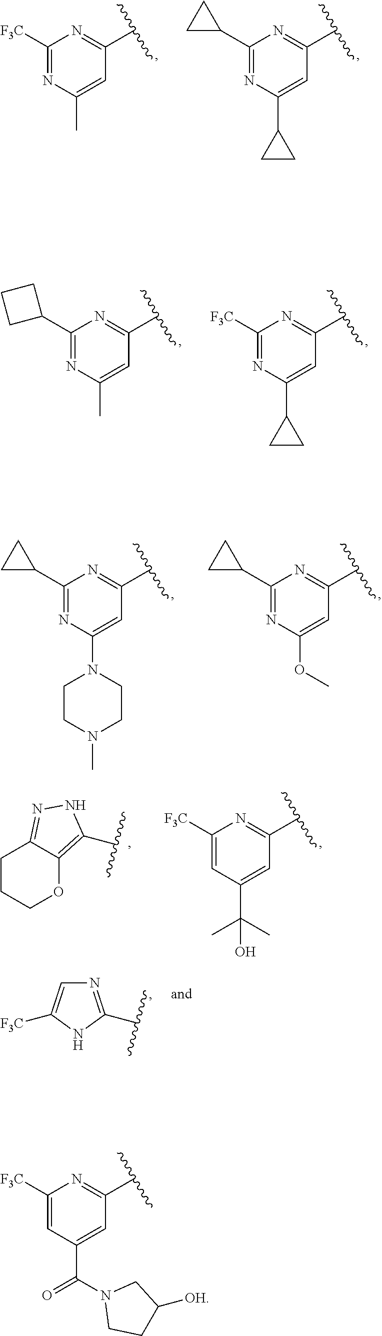

158. The pharmaceutical composition of any one of claims 124-126, wherein ring A is selected from the group consisting of

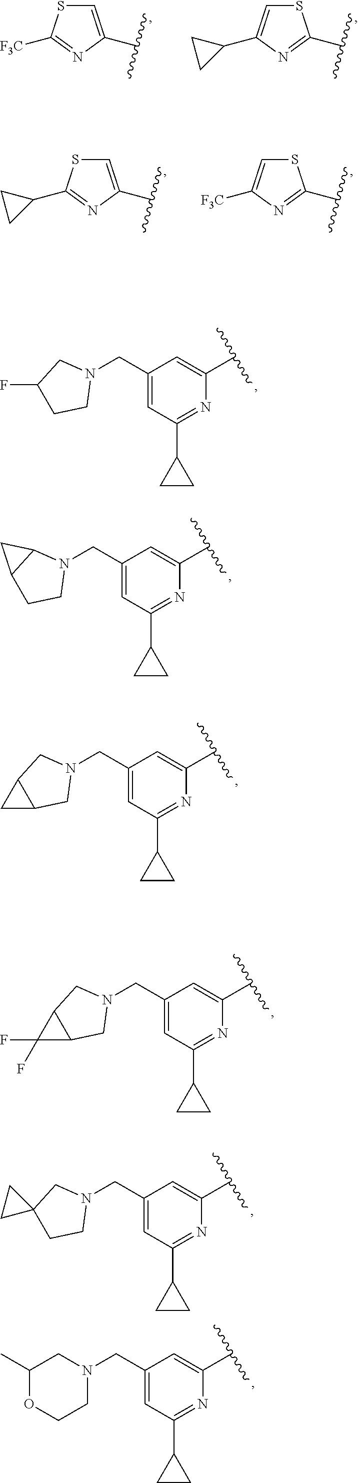

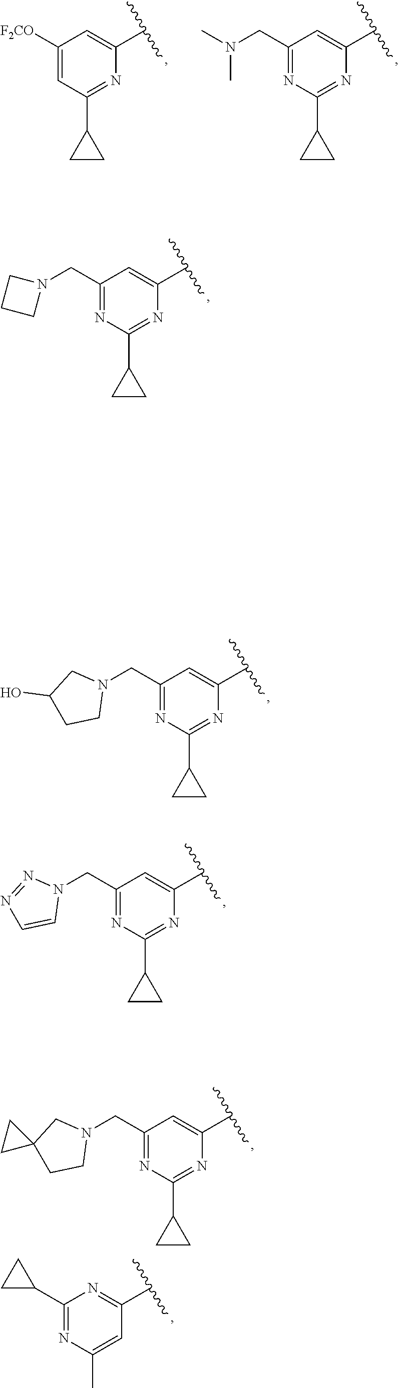

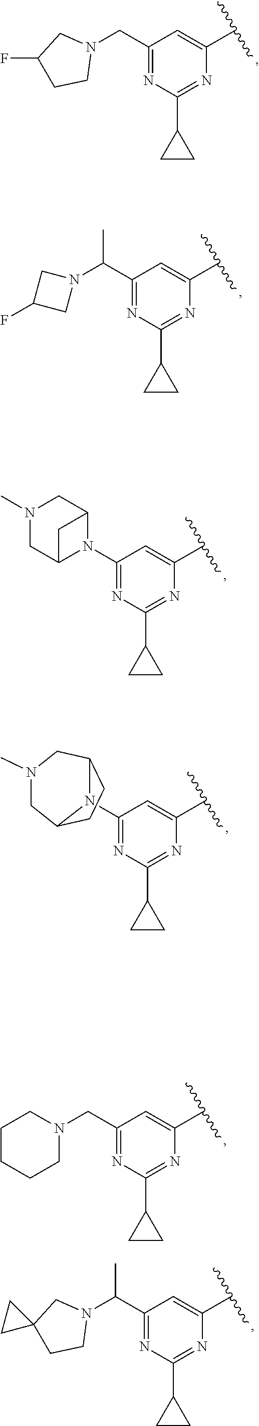

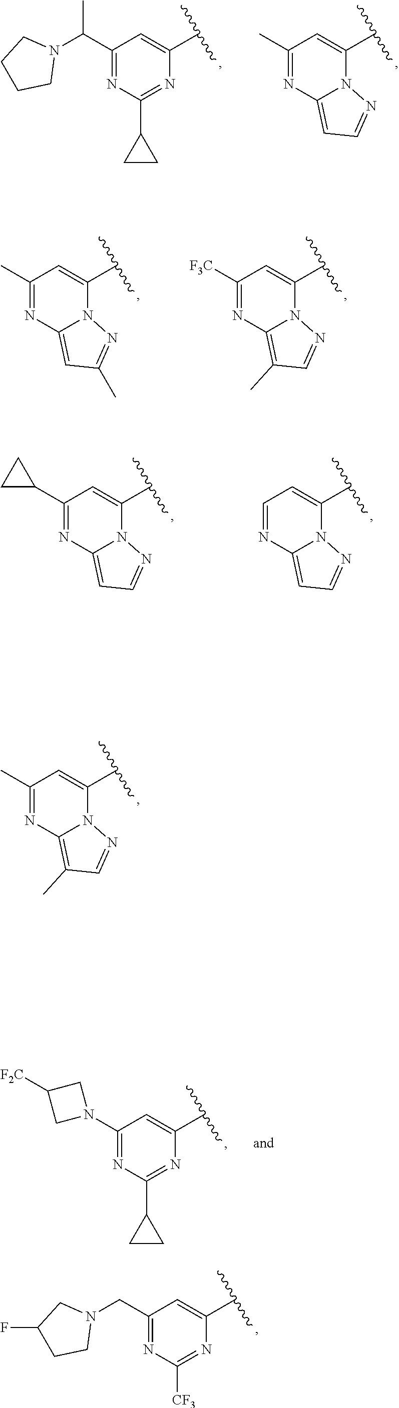

159. The pharmaceutical composition of any one of claims 124-158, wherein R⁶ is H.

160. The pharmaceutical composition of any one of claims 124-126, wherein R⁶ and the amide group to which R⁶ is connected attach to Ring A to form a five-membered lactam ring fused to Ring A, such that the fragment 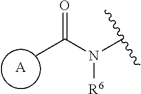
is

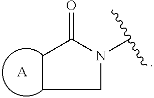

161. The pharmaceutical composition of claim 160, wherein 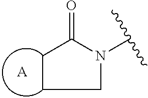
is selected from the group consisting of 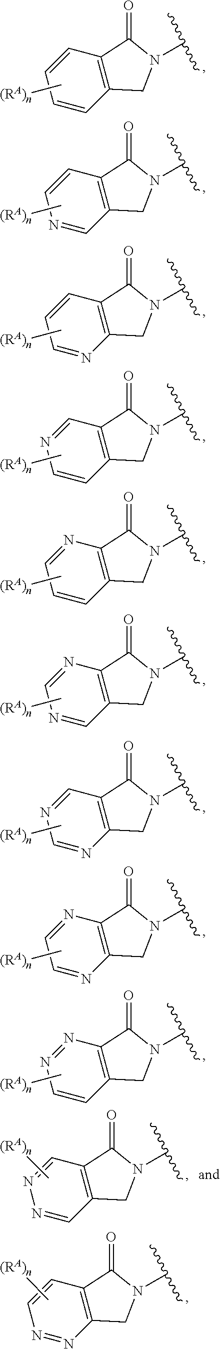
wherein:
n is 0, 1, or 2; and
each R^{A} is independently selected from the group consisting of H, F, Cl, Br, I, —OH, —CN, C₁-C₈ alkyl, —O—C₁-C₈ alkyl, —C₁-C₈ alkyl-OH, C₃-C₈ cycloalkyl, C₁-C₈ haloalkyl, —O—C₁-C₈ haloalkyl, —O—C₁-C₈ haloalkyl-OH, and —C(═O)NR^{c}R^{d}, wherein R^{c} and R^{d} are taken together with the nitrogen to which they are attached to form a three- to eight-membered heterocyclic ring optionally substituted with one, two, or three substituents independently selected from the group consisting of —OH, oxo, —CN, C₁-C₄ alkyl, —C(═O)—C₁-C₄ alkyl, F, Cl, Br, and I.

162. The pharmaceutical composition of claim 160, wherein 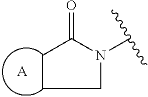
is 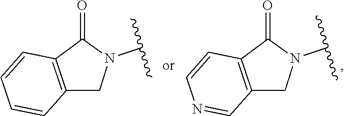 [SMILES: *N1Cc2ccccc2C1=O ; *N1Cc2cnccc2C1=O]
wherein ring A is optionally substituted with one, two, or three groups independently selected from the group consisting of F, Cl, Br, I, —OH, —CN, C₁-C₈ alkyl, —O—C₁-C₈ alkyl, —C₁-C₈ alkyl-OH, C₃-C₈ cycloalkyl, C₁-C₈ haloalkyl, —O—C₁-C₈ haloalkyl, —O—C₁-C₈ haloalkyl-OH, and —C(═O)NR^{c}R^{d}, wherein R^{c} and R^{d} are taken together with the nitrogen to which they are attached to form a three- to eight-membered heterocyclic ring optionally substituted with one, two, or three substituents independently selected from the group consisting of —OH, oxo, C₁-C₄ alkyl, —C(═O)—C₁-C₄ alkyl, F, Cl, Br, and I.

163. The pharmaceutical composition of claim 160, wherein 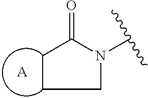
is selected from the group consisting of

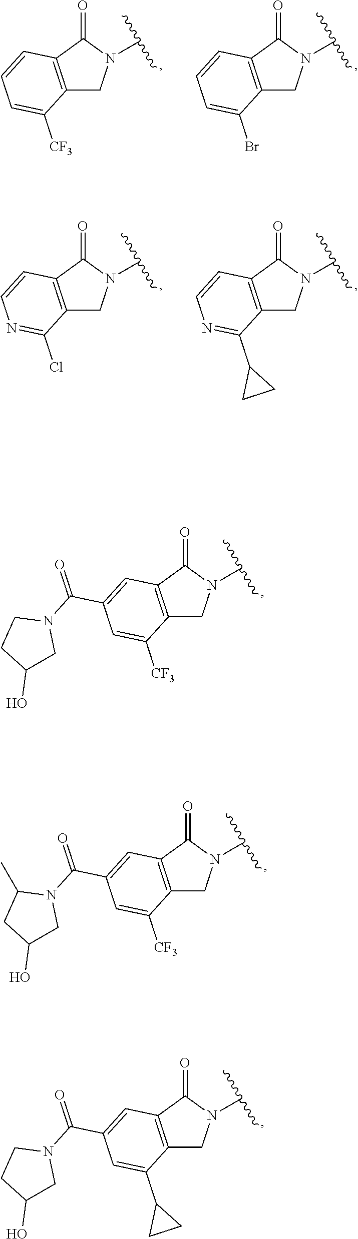

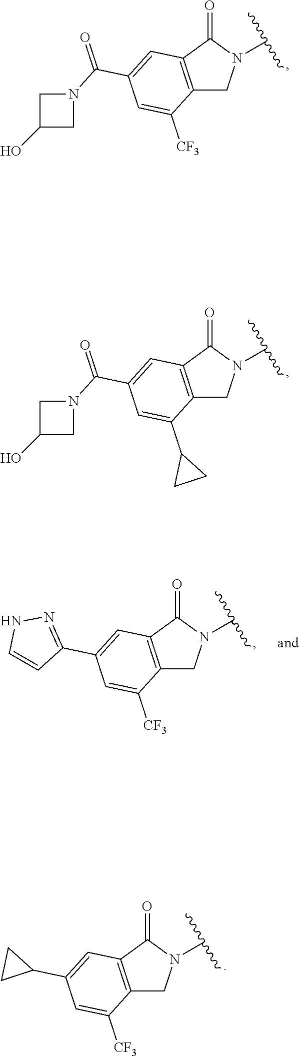 [SMILES: *N1Cc2c(cc(C(=O)N3CC(O)C3)cc2C(F)(F)F)C1=O ; *N1Cc2c(cc(C(=O)N3CC(O)C3)cc2C2CC2)C1=O ; *N1Cc2c(cc(-c3cc[nH]n3)cc2C(F)(F)F)C1=O ; *N1Cc2c(cc(C3CC3)cc2C(F)(F)F)C1=O]

164. The pharmaceutical composition of claim 160, wherein 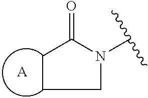
is selected from the group consisting of

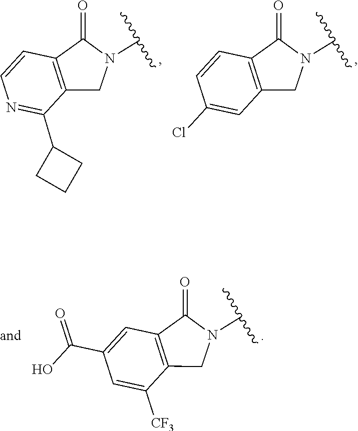 [SMILES: *N1Cc2c(ccnc2C2CCC2)C1=O ; *N1Cc2cc(Cl)ccc2C1=O ; *N1Cc2c(cc(C(=O)O)cc2C(F)(F)F)C1=O]

165. The pharmaceutical composition of claim 160, wherein 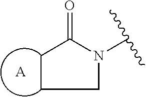
is selected from the group consisting of

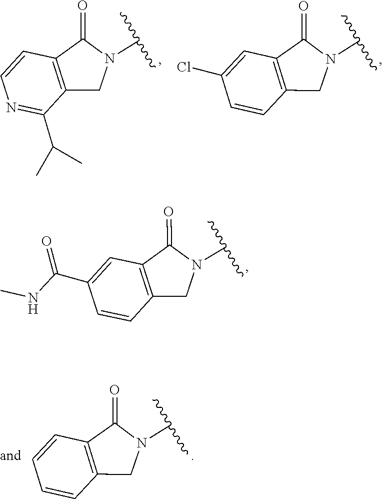 [SMILES: *N1Cc2c(ccnc2C(C)C)C1=O ; *N1Cc2ccc(Cl)cc2C1=O ; *N1Cc2ccc(C(=O)NC)cc2C1=O ; *N1Cc2ccccc2C1=O]

166. The pharmaceutical composition of claim 160, wherein 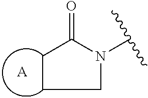
is selected from the group consisting of

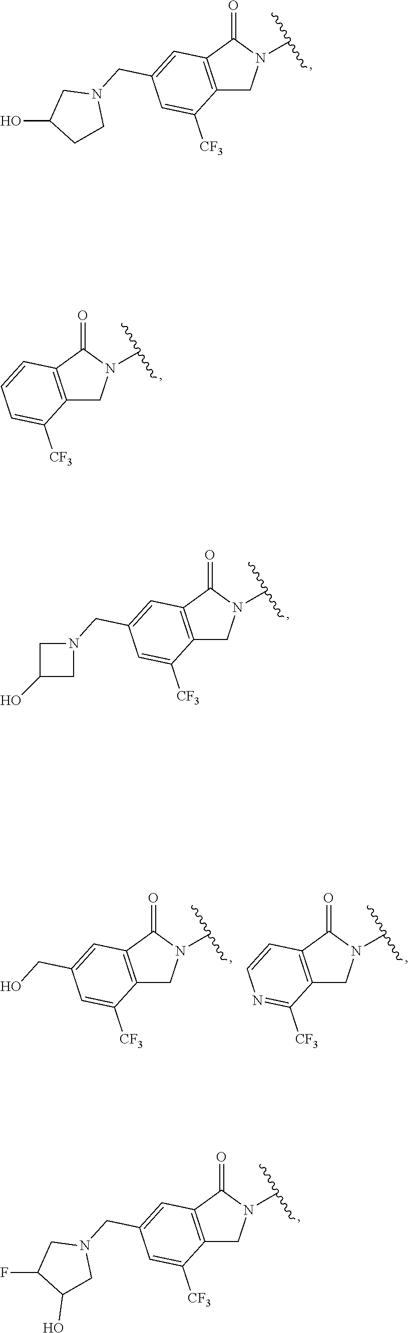 [SMILES: *N1Cc2c(cc(CN3CCC(O)C3)cc2C(F)(F)F)C1=O ; *N1Cc2c(cccc2C(F)(F)F)C1=O ; *N1Cc2c(cc(CN3CC(O)C3)cc2C(F)(F)F)C1=O ; *N1Cc2c(cc(CO)cc2C(F)(F)F)C1=O ; *N1Cc2c(ccnc2C(F)(F)F)C1=O ; *N1Cc2c(cc(CN3CC(O)C(F)C3)cc2C(F)(F)F)C1=O]

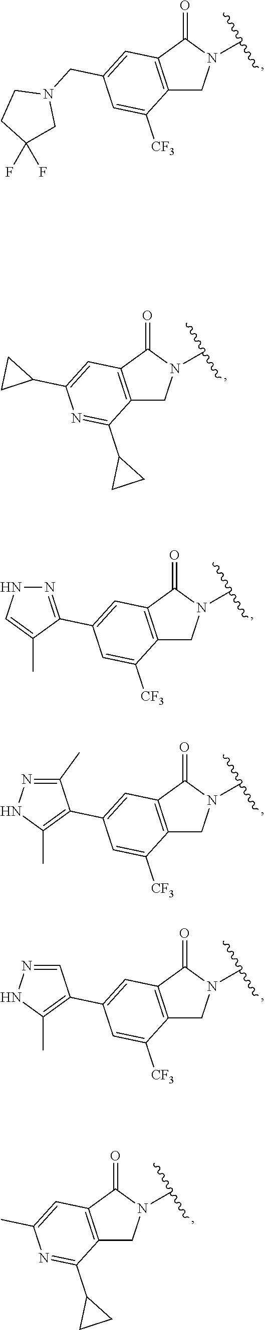 [SMILES: *N1Cc2c(cc(CN3CCC(F)(F)C3)cc2C(F)(F)F)C1=O ; *N1Cc2c(cc(C3CC3)nc2C2CC2)C1=O ; *N1Cc2c(cc(-c3n[nH]cc3C)cc2C(F)(F)F)C1=O ; *N1Cc2c(cc(-c3c(C)n[nH]c3C)cc2C(F)(F)F)C1=O ; *N1Cc2c(cc(-c3cn[nH]c3C)cc2C(F)(F)F)C1=O ; *N1Cc2c(cc(C)nc2C2CC2)C1=O]

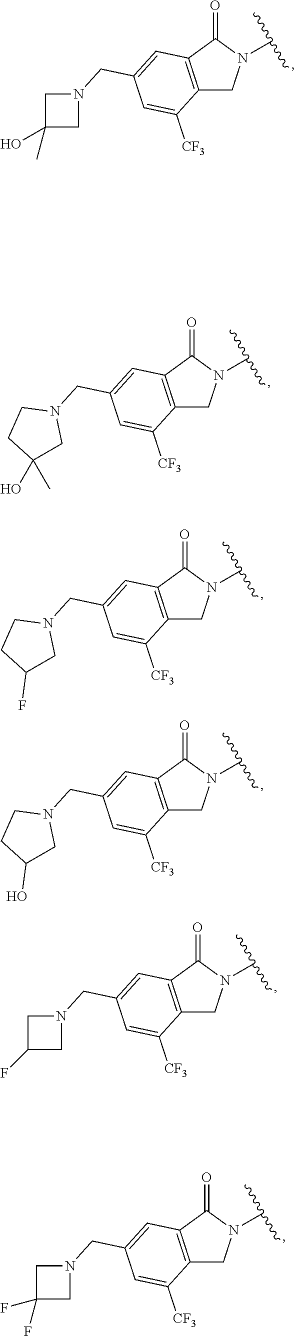 [SMILES: *N1Cc2c(cc(CN3CC(C)(O)C3)cc2C(F)(F)F)C1=O ; *N1Cc2c(cc(CN3CCC(C)(O)C3)cc2C(F)(F)F)C1=O ; *N1Cc2c(cc(CN3CCC(F)C3)cc2C(F)(F)F)C1=O ; *N1Cc2c(cc(CN3CCC(O)C3)cc2C(F)(F)F)C1=O ; *N1Cc2c(cc(CN3CC(F)C3)cc2C(F)(F)F)C1=O ; *N1Cc2c(cc(CN3CC(F)(F)C3)cc2C(F)(F)F)C1=O]

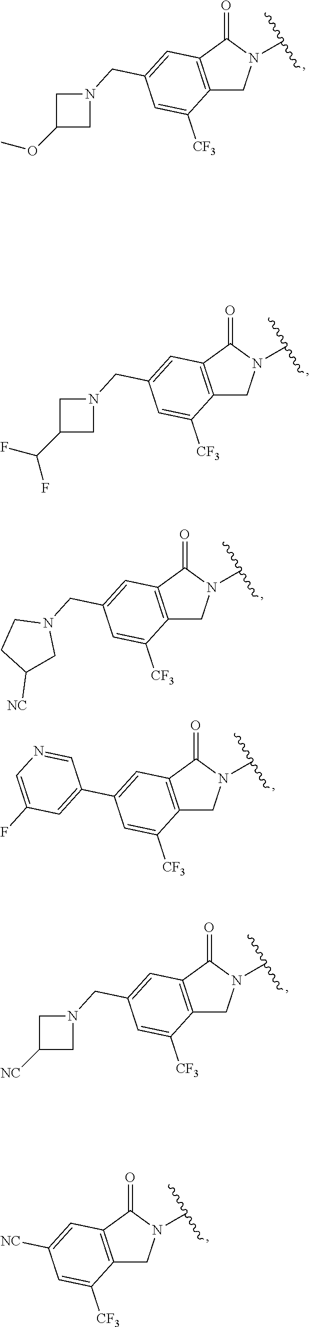 [SMILES: *N1Cc2c(cc(CN3CC(OC)C3)cc2C(F)(F)F)C1=O ; *N1Cc2c(cc(CN3CC(C(F)F)C3)cc2C(F)(F)F)C1=O ; *N1Cc2c(cc(CN3CCC(C#N)C3)cc2C(F)(F)F)C1=O ; *N1Cc2c(cc(-c3cncc(F)c3)cc2C(F)(F)F)C1=O ; *N1Cc2c(cc(CN3CC(C#N)C3)cc2C(F)(F)F)C1=O ; *N1Cc2c(cc(C#N)cc2C(F)(F)F)C1=O]

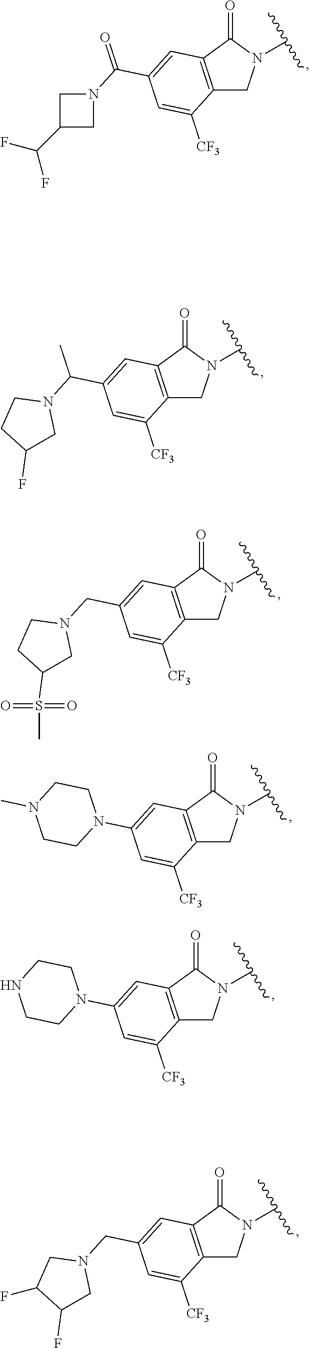

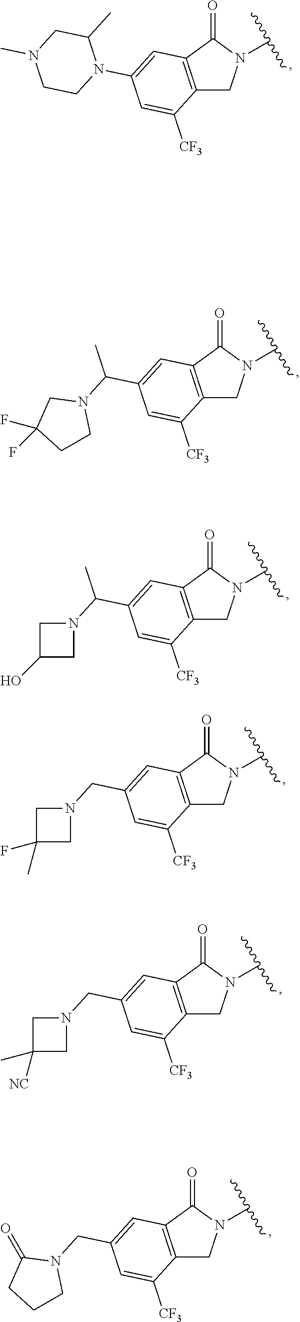 [SMILES: *N1Cc2c(cc(N3CCN(C)CC3C)cc2C(F)(F)F)C1=O ; *N1Cc2c(cc(C(C)N3CCC(F)(F)C3)cc2C(F)(F)F)C1=O ; *N1Cc2c(cc(C(C)N3CC(O)C3)cc2C(F)(F)F)C1=O ; *N1Cc2c(cc(CN3CC(C)(F)C3)cc2C(F)(F)F)C1=O ; *N1Cc2c(cc(CN3CC(C)(C#N)C3)cc2C(F)(F)F)C1=O ; *N1Cc2c(cc(CN3CCCC3=O)cc2C(F)(F)F)C1=O]

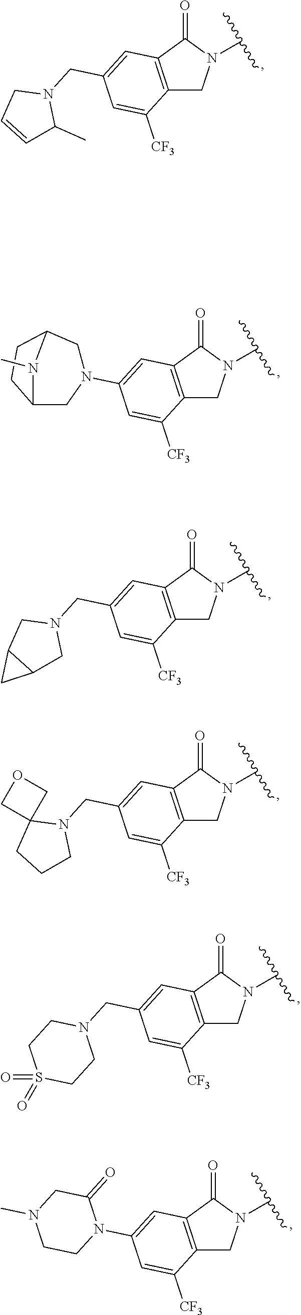 [SMILES: *N1Cc2c(cc(CN3CC=CC3C)cc2C(F)(F)F)C1=O ; *N1Cc2c(cc(N3CC4CCC(C3)N4C)cc2C(F)(F)F)C1=O ; *N1Cc2c(cc(CN3CC4CC4C3)cc2C(F)(F)F)C1=O ; *N1Cc2c(cc(CN3CCCC34COC4)cc2C(F)(F)F)C1=O ; *N1Cc2c(cc(CN3CCS(=O)(=O)CC3)cc2C(F)(F)F)C1=O ; *N1Cc2c(cc(N3CCN(C)CC3=O)cc2C(F)(F)F)C1=O]

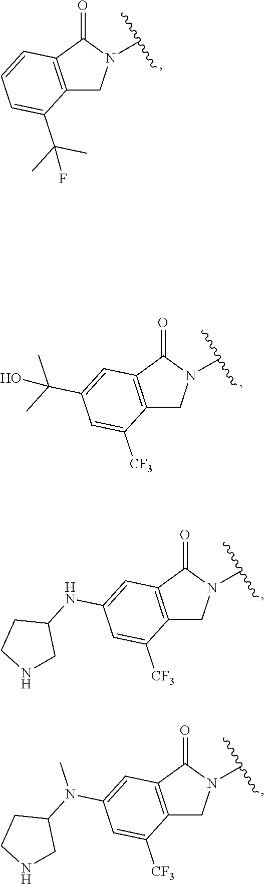 [SMILES: *N1Cc2c(cccc2C(C)(C)F)C1=O ; *N1Cc2c(cc(C(C)(C)O)cc2C(F)(F)F)C1=O ; *N1Cc2c(cc(NC3CCNC3)cc2C(F)(F)F)C1=O ; *N1Cc2c(cc(N(C)C3CCNC3)cc2C(F)(F)F)C1=O]

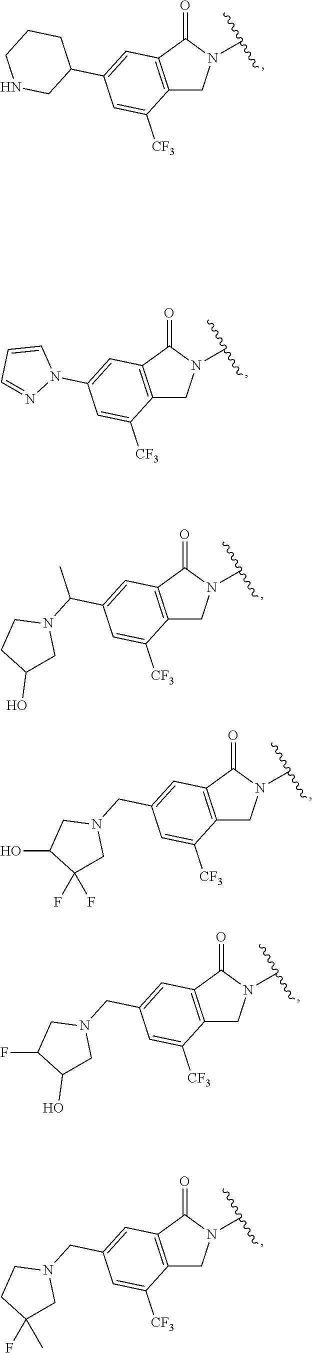 [SMILES: *N1Cc2c(cc(C3CCCNC3)cc2C(F)(F)F)C1=O ; *N1Cc2c(cc(-n3cccn3)cc2C(F)(F)F)C1=O ; *N1Cc2c(cc(C(C)N3CCC(O)C3)cc2C(F)(F)F)C1=O ; *N1Cc2c(cc(CN3CC(O)C(F)(F)C3)cc2C(F)(F)F)C1=O ; *N1Cc2c(cc(CN3CC(O)C(F)C3)cc2C(F)(F)F)C1=O ; *N1Cc2c(cc(CN3CCC(C)(F)C3)cc2C(F)(F)F)C1=O]

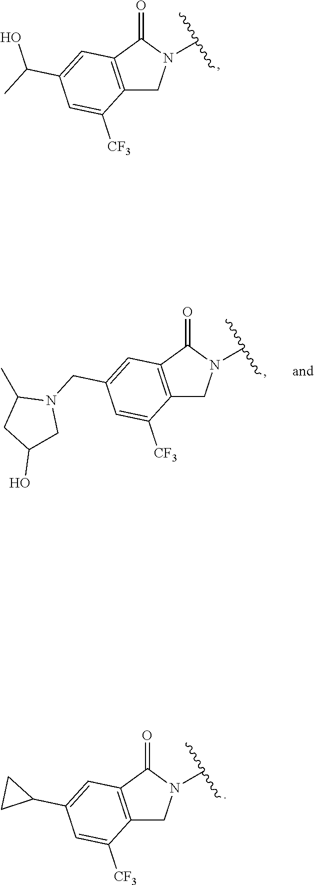 [SMILES: *N1Cc2c(cc(C(C)O)cc2C(F)(F)F)C1=O ; *N1Cc2c(cc(CN3CC(O)CC3C)cc2C(F)(F)F)C1=O ; *N1Cc2c(cc(C3CC3)cc2C(F)(F)F)C1=O]

167. The pharmaceutical composition of claim 160, wherein 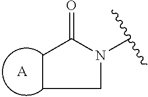
is selected from the group consisting of

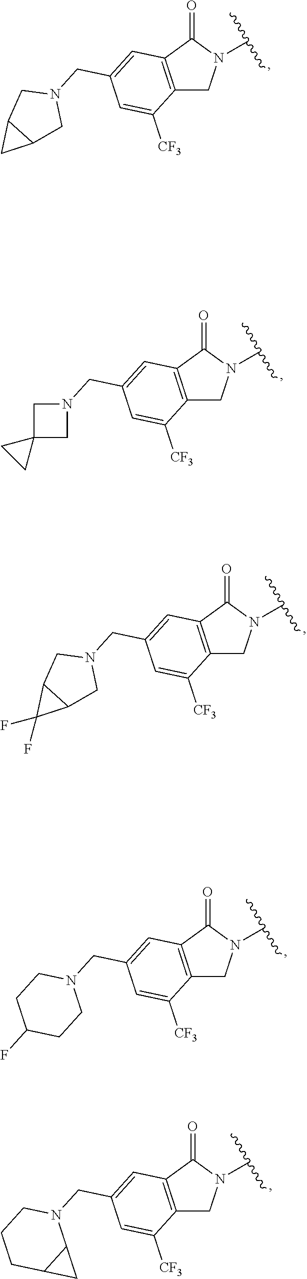 [SMILES: *N1Cc2c(cc(CN3CC4CC4C3)cc2C(F)(F)F)C1=O ; *N1Cc2c(cc(CN3CC4(CC4)C3)cc2C(F)(F)F)C1=O ; *N1Cc2c(cc(CN3CC4C(C3)C4(F)F)cc2C(F)(F)F)C1=O ; *N1Cc2c(cc(CN3CCC(F)CC3)cc2C(F)(F)F)C1=O ; *N1Cc2c(cc(CN3CCCC4CC43)cc2C(F)(F)F)C1=O]

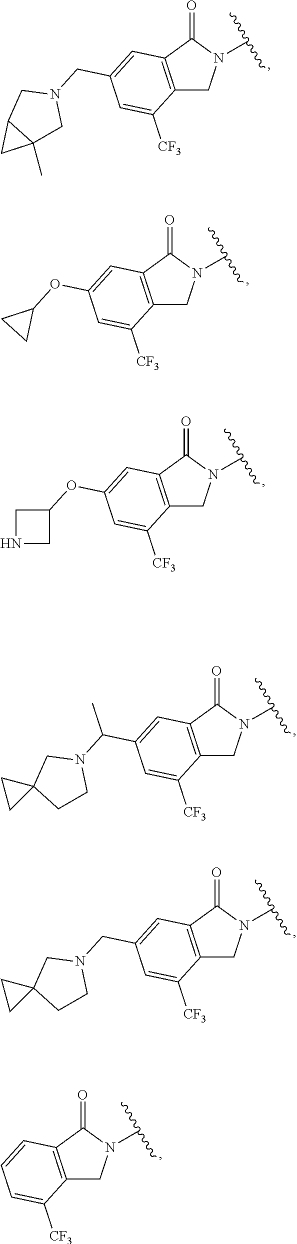 [SMILES: *N1Cc2c(cc(CN3CC4CC4(C)C3)cc2C(F)(F)F)C1=O ; *N1Cc2c(cc(OC3CC3)cc2C(F)(F)F)C1=O ; *N1Cc2c(cc(OC3CNC3)cc2C(F)(F)F)C1=O ; *N1Cc2c(cc(C(C)N3CCC4(CC4)C3)cc2C(F)(F)F)C1=O ; *N1Cc2c(cc(CN3CCC4(CC4)C3)cc2C(F)(F)F)C1=O ; *N1Cc2c(cccc2C(F)(F)F)C1=O]

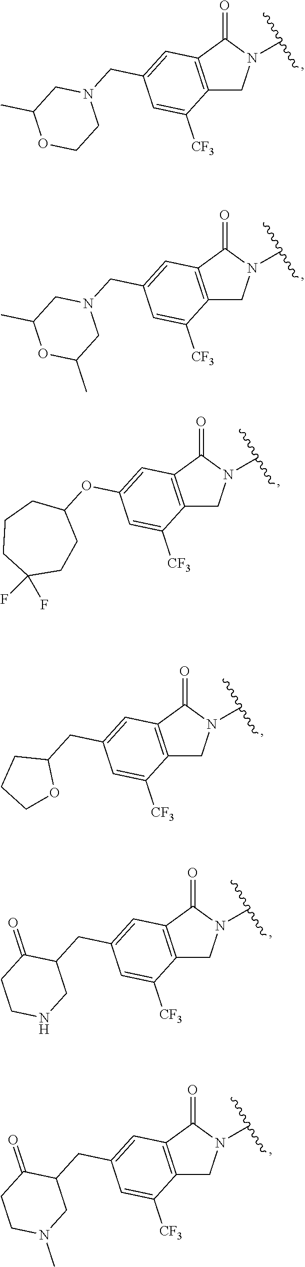 [SMILES: *N1Cc2c(cc(CN3CCOC(C)C3)cc2C(F)(F)F)C1=O ; *N1Cc2c(cc(CN3CC(C)OC(C)C3)cc2C(F)(F)F)C1=O ; *N1Cc2c(cc(OC3CCCC(F)(F)CC3)cc2C(F)(F)F)C1=O ; *N1Cc2c(cc(CC3CCCO3)cc2C(F)(F)F)C1=O ; *N1Cc2c(cc(CC3CNCCC3=O)cc2C(F)(F)F)C1=O ; *N1Cc2c(cc(CC3CN(C)CCC3=O)cc2C(F)(F)F)C1=O]

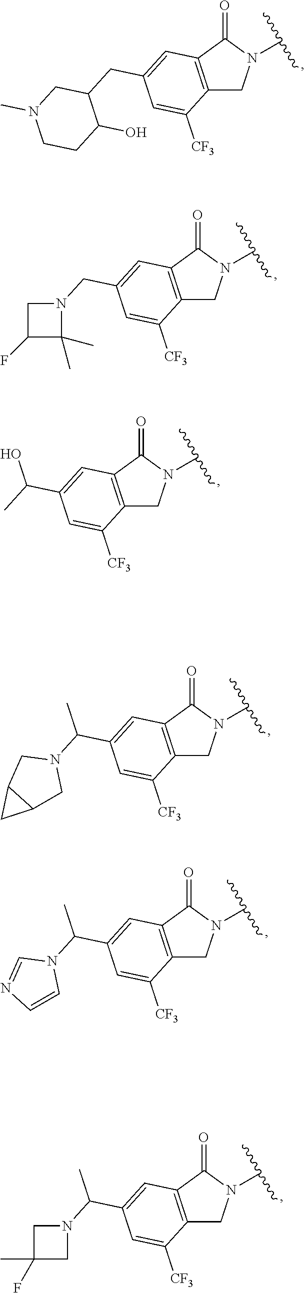 [SMILES: *N1Cc2c(cc(CC3CN(C)CCC3O)cc2C(F)(F)F)C1=O ; *N1Cc2c(cc(CN3CC(F)C3(C)C)cc2C(F)(F)F)C1=O ; *N1Cc2c(cc(C(C)O)cc2C(F)(F)F)C1=O ; *N1Cc2c(cc(C(C)N3CC4CC4C3)cc2C(F)(F)F)C1=O ; *N1Cc2c(cc(C(C)n3ccnc3)cc2C(F)(F)F)C1=O ; *N1Cc2c(cc(C(C)N3CC(C)(F)C3)cc2C(F)(F)F)C1=O]

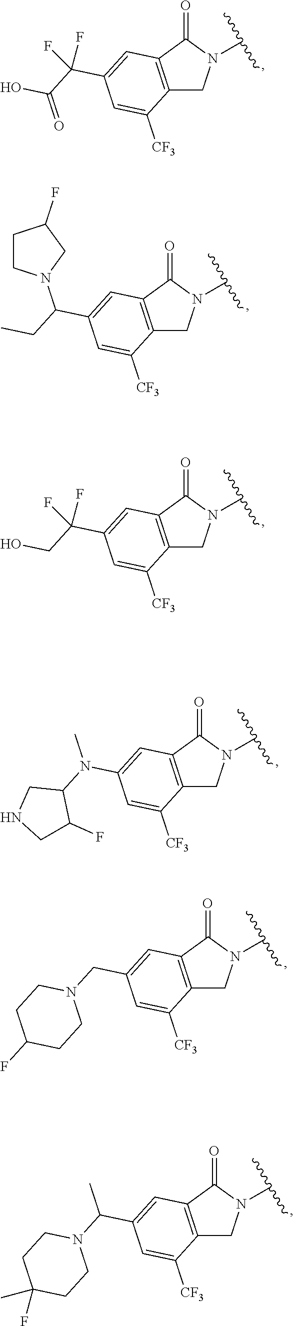 [SMILES: *N1Cc2c(cc(C(F)(F)C(=O)O)cc2C(F)(F)F)C1=O ; *N1Cc2c(cc(C(CC)N3CCC(F)C3)cc2C(F)(F)F)C1=O ; *N1Cc2c(cc(C(F)(F)CO)cc2C(F)(F)F)C1=O ; *N1Cc2c(cc(N(C)C3CNCC3F)cc2C(F)(F)F)C1=O ; *N1Cc2c(cc(CN3CCC(F)CC3)cc2C(F)(F)F)C1=O ; *N1Cc2c(cc(C(C)N3CCC(C)(F)CC3)cc2C(F)(F)F)C1=O]

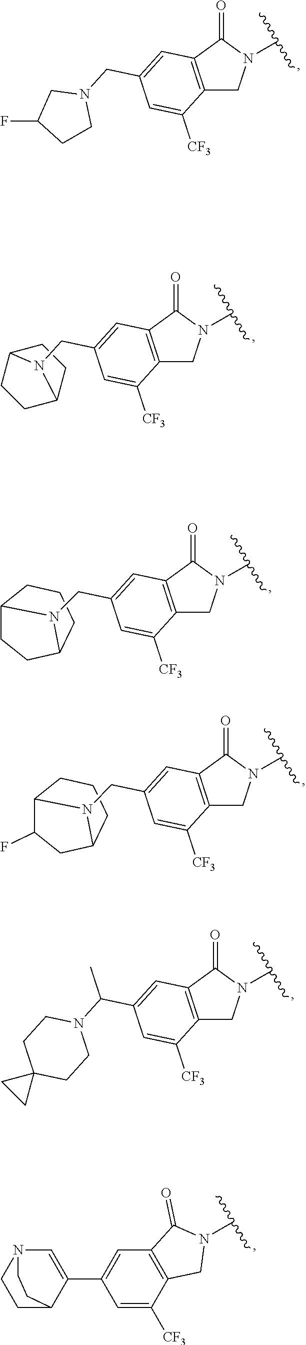 [SMILES: *N1Cc2c(cc(CN3CCC(F)C3)cc2C(F)(F)F)C1=O ; *N1Cc2c(cc(CN3C4CCC3CC4)cc2C(F)(F)F)C1=O ; *N1Cc2c(cc(CN3C4CCCC3CC4)cc2C(F)(F)F)C1=O ; *N1Cc2c(cc(CN3C4CCCC3C(F)C4)cc2C(F)(F)F)C1=O ; *N1Cc2c(cc(C(C)N3CCC4(CC3)CC4)cc2C(F)(F)F)C1=O ; *N1Cc2c(cc(C3=CN4CCC3CC4)cc2C(F)(F)F)C1=O]

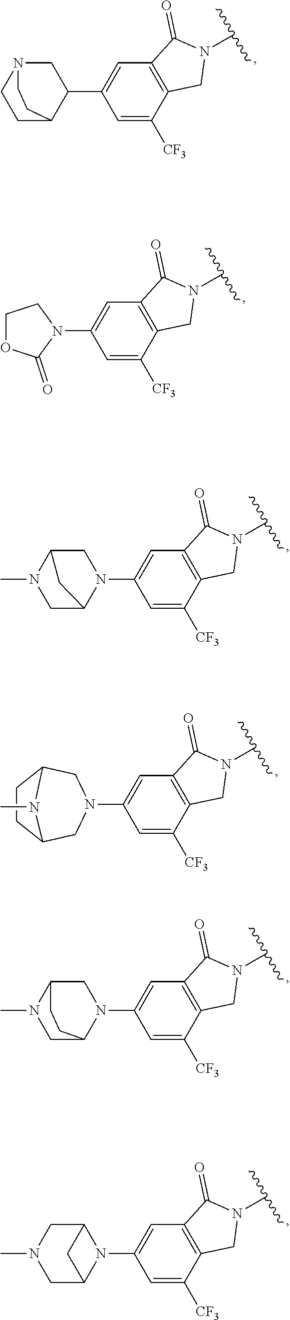 [SMILES: *N1Cc2c(cc(C3CN4CCC3CC4)cc2C(F)(F)F)C1=O ; *N1Cc2c(cc(N3CCOC3=O)cc2C(F)(F)F)C1=O ; *N1Cc2c(cc(N3CC4CC3CN4C)cc2C(F)(F)F)C1=O ; *N1Cc2c(cc(N3CC4CCC(C3)N4C)cc2C(F)(F)F)C1=O ; *N1Cc2c(cc(N3CC4CCC3CN4C)cc2C(F)(F)F)C1=O ; *N1Cc2c(cc(N3C4CC3CN(C)C4)cc2C(F)(F)F)C1=O]

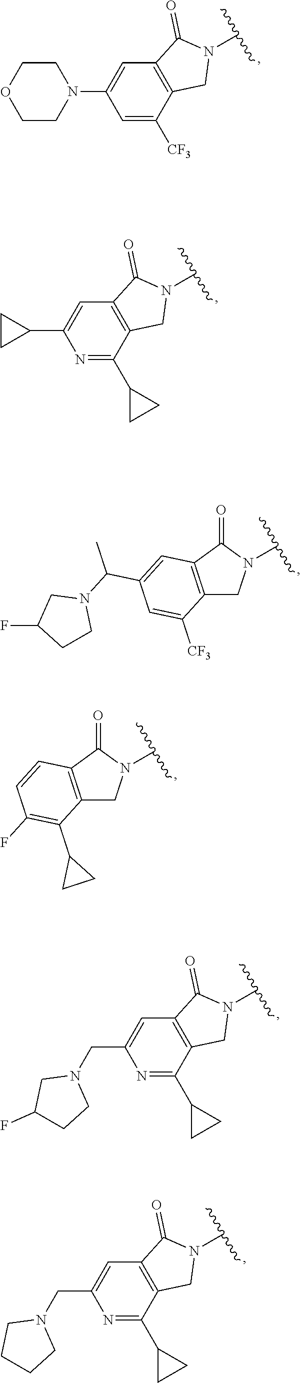 [SMILES: *N1Cc2c(cc(N3CCOCC3)cc2C(F)(F)F)C1=O ; *N1Cc2c(cc(C3CC3)nc2C2CC2)C1=O ; *N1Cc2c(cc(C(C)N3CCC(F)C3)cc2C(F)(F)F)C1=O ; *N1Cc2c(ccc(F)c2C2CC2)C1=O ; *N1Cc2c(cc(CN3CCC(F)C3)nc2C2CC2)C1=O ; *N1Cc2c(cc(CN3CCCC3)nc2C2CC2)C1=O]

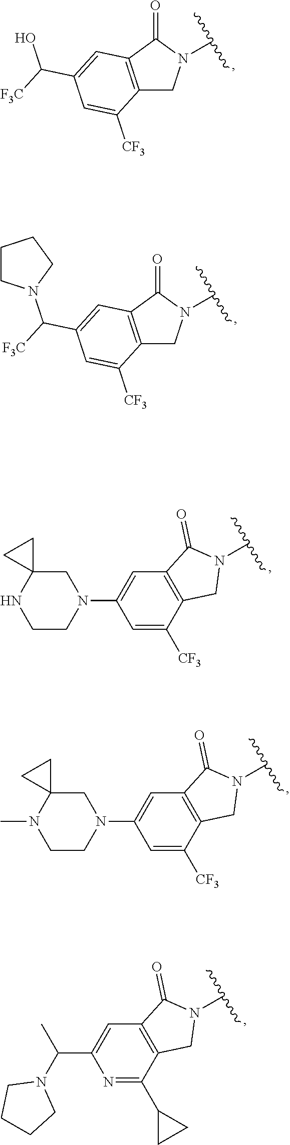 [SMILES: *N1Cc2c(cc(C(O)C(F)(F)F)cc2C(F)(F)F)C1=O ; *N1Cc2c(cc(C(N3CCCC3)C(F)(F)F)cc2C(F)(F)F)C1=O ; *N1Cc2c(cc(N3CCNC4(CC4)C3)cc2C(F)(F)F)C1=O ; *N1Cc2c(cc(N3CCN(C)C4(CC4)C3)cc2C(F)(F)F)C1=O ; *N1Cc2c(cc(C(C)N3CCCC3)nc2C2CC2)C1=O]

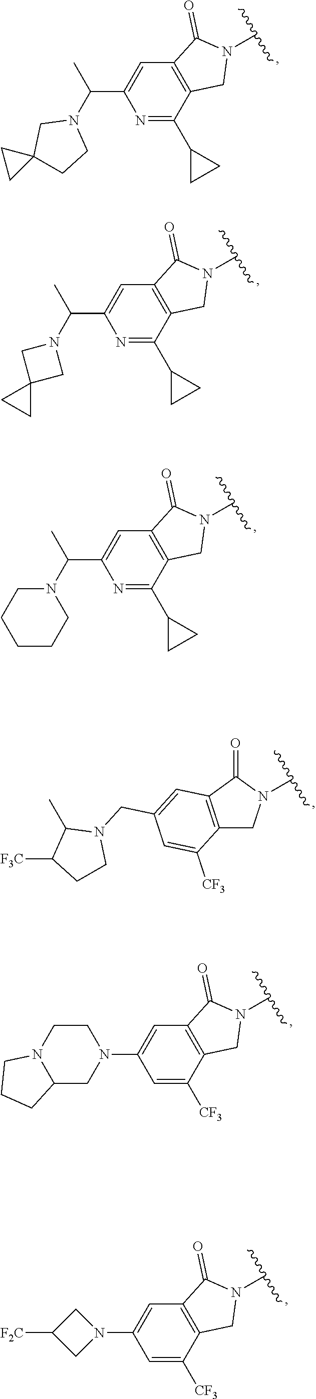

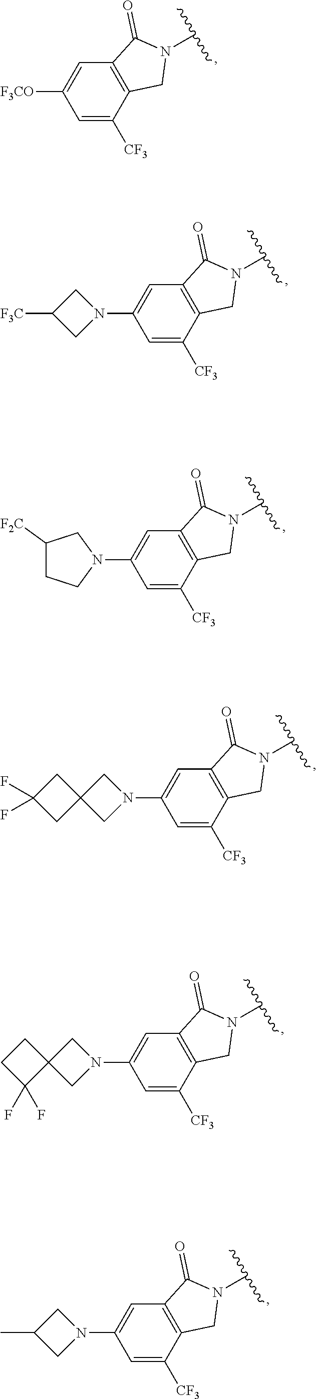

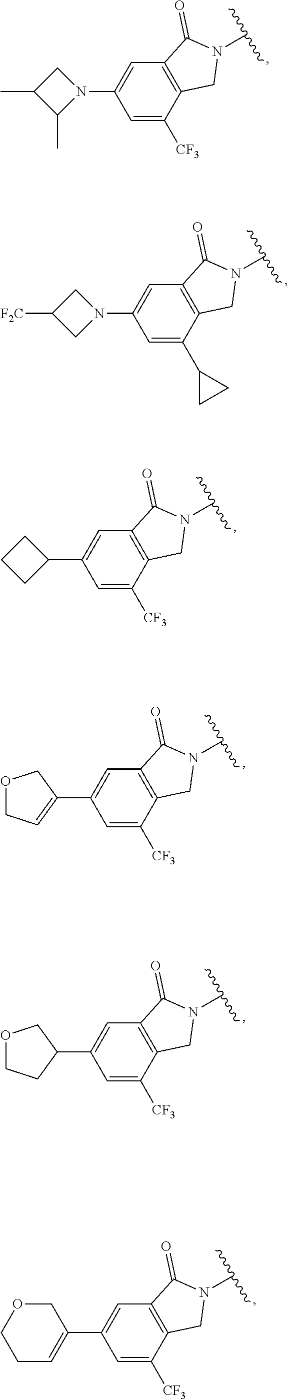

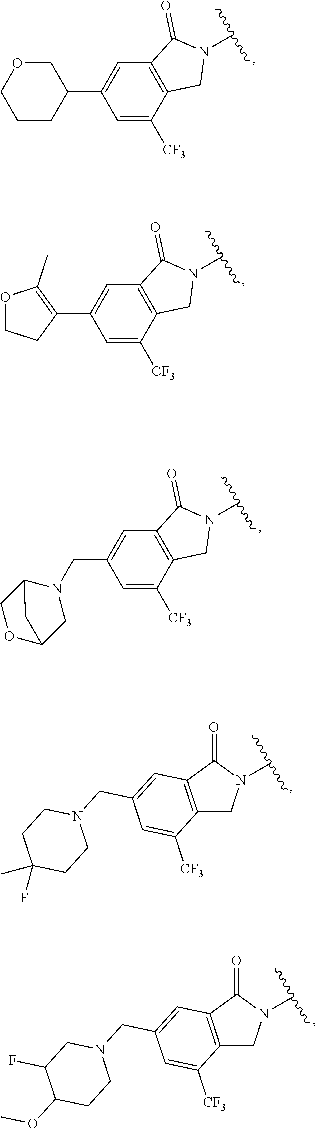 [SMILES: *N1Cc2c(cc(C3CCCOC3)cc2C(F)(F)F)C1=O ; *N1Cc2c(cc(C3=C(C)OCC3)cc2C(F)(F)F)C1=O ; *N1Cc2c(cc(CN3CC4CC3CO4)cc2C(F)(F)F)C1=O ; *N1Cc2c(cc(CN3CCC(C)(F)CC3)cc2C(F)(F)F)C1=O ; *N1Cc2c(cc(CN3CCC(OC)C(F)C3)cc2C(F)(F)F)C1=O]

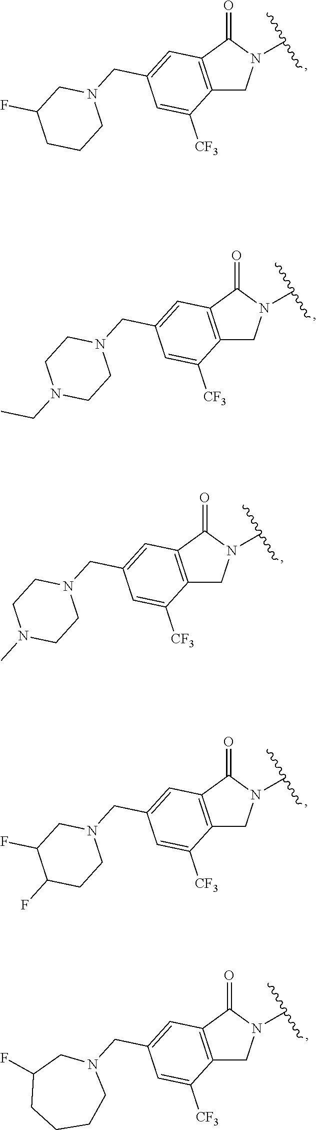 [SMILES: *N1Cc2c(cc(CN3CCCC(F)C3)cc2C(F)(F)F)C1=O ; *N1Cc2c(cc(CN3CCN(CC)CC3)cc2C(F)(F)F)C1=O ; *N1Cc2c(cc(CN3CCN(C)CC3)cc2C(F)(F)F)C1=O ; *N1Cc2c(cc(CN3CCC(F)C(F)C3)cc2C(F)(F)F)C1=O ; *N1Cc2c(cc(CN3CCCCC(F)C3)cc2C(F)(F)F)C1=O]

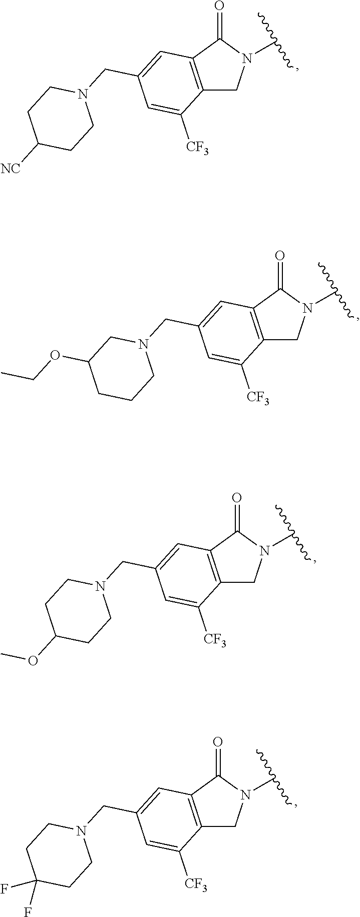 [SMILES: *N1Cc2c(cc(CN3CCC(C#N)CC3)cc2C(F)(F)F)C1=O ; *N1Cc2c(cc(CN3CCCC(OCC)C3)cc2C(F)(F)F)C1=O ; *N1Cc2c(cc(CN3CCC(OC)CC3)cc2C(F)(F)F)C1=O ; *N1Cc2c(cc(CN3CCC(F)(F)CC3)cc2C(F)(F)F)C1=O]

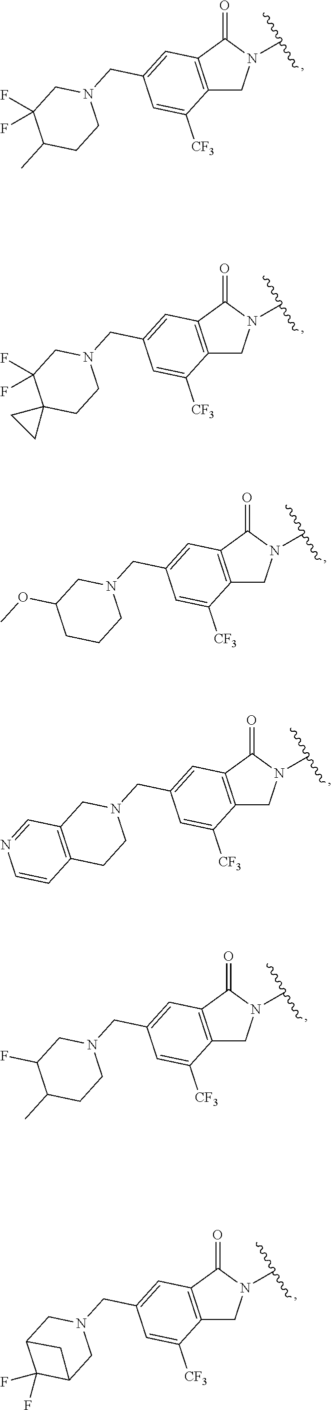 [SMILES: *N1Cc2c(cc(CN3CCC(C)C(F)(F)C3)cc2C(F)(F)F)C1=O ; *N1Cc2c(cc(CN3CCC4(CC4)C(F)(F)C3)cc2C(F)(F)F)C1=O ; *N1Cc2c(cc(CN3CCCC(OC)C3)cc2C(F)(F)F)C1=O ; *N1Cc2c(cc(CN3CCc4ccncc4C3)cc2C(F)(F)F)C1=O ; *N1Cc2c(cc(CN3CCC(C)C(F)C3)cc2C(F)(F)F)C1=O ; *N1Cc2c(cc(CN3CC4CC(C3)C4(F)F)cc2C(F)(F)F)C1=O]

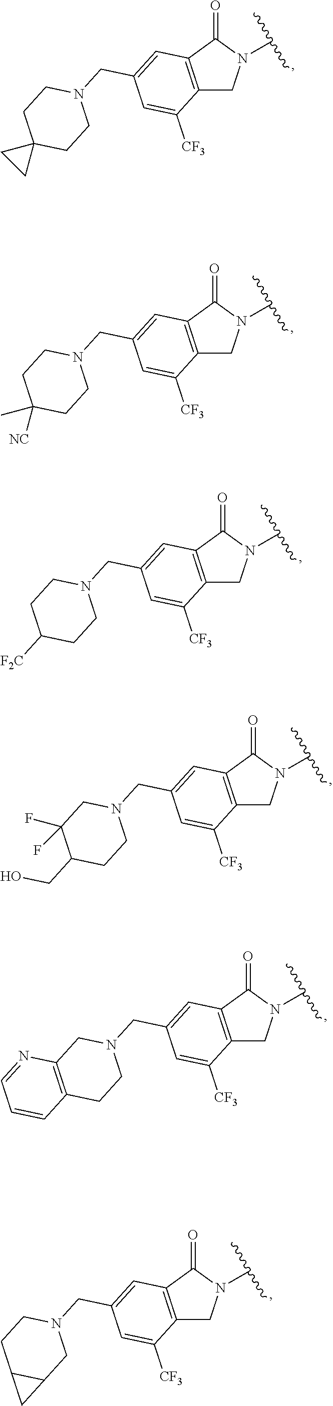

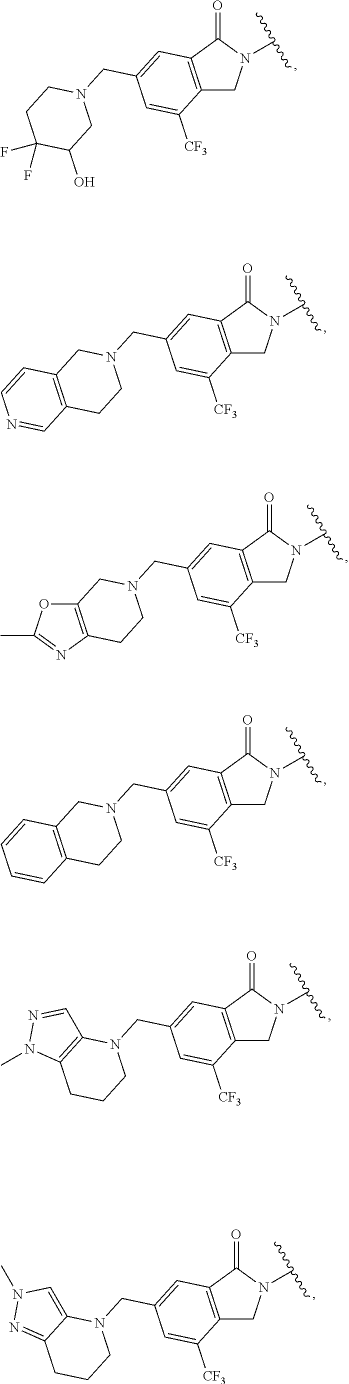 [SMILES: *N1Cc2c(cc(CN3CCC(F)(F)C(O)C3)cc2C(F)(F)F)C1=O ; *N1Cc2c(cc(CN3CCc4cnccc4C3)cc2C(F)(F)F)C1=O ; *N1Cc2c(cc(CN3CCc4nc(C)oc4C3)cc2C(F)(F)F)C1=O ; *N1Cc2c(cc(CN3CCc4ccccc4C3)cc2C(F)(F)F)C1=O ; *N1Cc2c(cc(CN3CCCc4c3cnn4C)cc2C(F)(F)F)C1=O ; *N1Cc2c(cc(CN3CCCc4nn(C)cc43)cc2C(F)(F)F)C1=O]

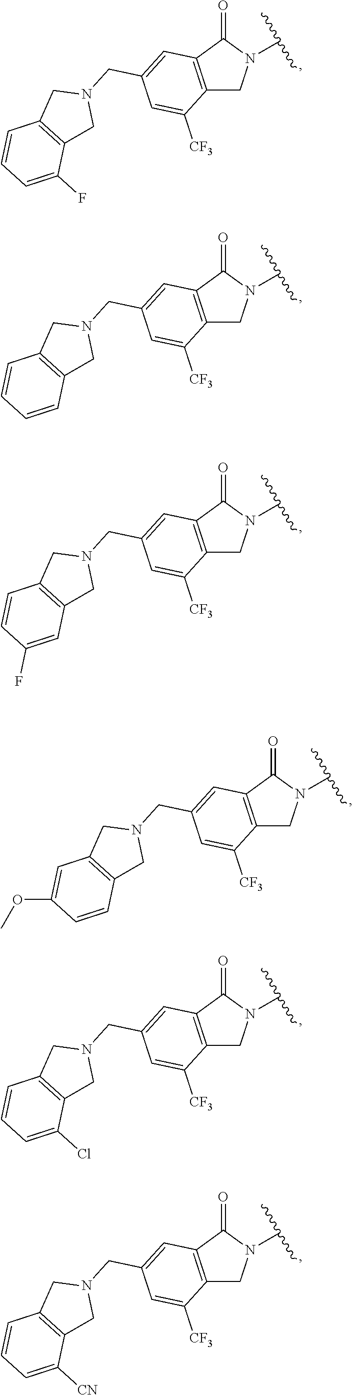 [SMILES: *N1Cc2c(cc(CN3Cc4cccc(F)c4C3)cc2C(F)(F)F)C1=O ; *N1Cc2c(cc(CN3Cc4ccccc4C3)cc2C(F)(F)F)C1=O ; *N1Cc2c(cc(CN3Cc4ccc(F)cc4C3)cc2C(F)(F)F)C1=O ; *N1Cc2c(cc(CN3Cc4ccc(OC)cc4C3)cc2C(F)(F)F)C1=O ; *N1Cc2c(cc(CN3Cc4cccc(Cl)c4C3)cc2C(F)(F)F)C1=O ; *N1Cc2c(cc(CN3Cc4cccc(C#N)c4C3)cc2C(F)(F)F)C1=O]

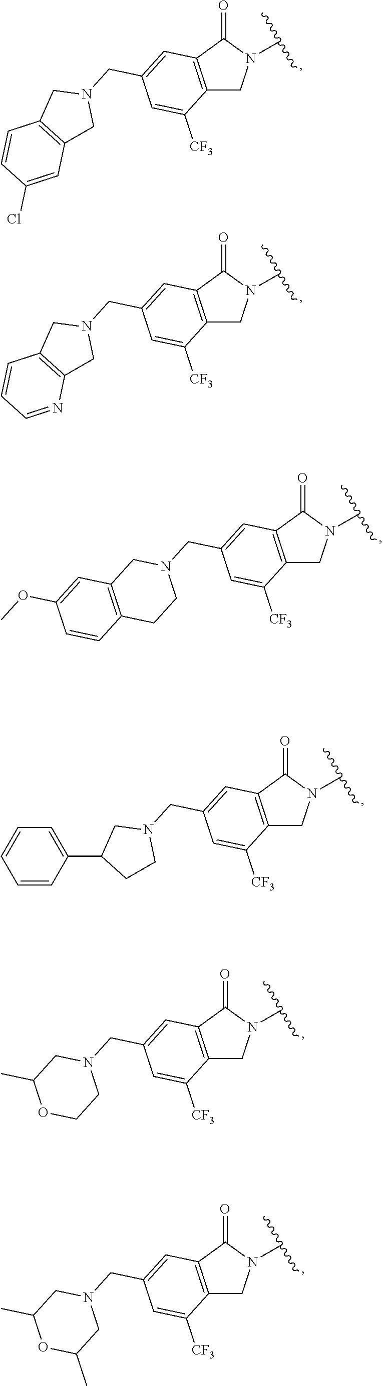 [SMILES: *N1Cc2c(cc(CN3Cc4ccc(Cl)cc4C3)cc2C(F)(F)F)C1=O ; *N1Cc2c(cc(CN3Cc4cccnc4C3)cc2C(F)(F)F)C1=O ; *N1Cc2c(cc(CN3CCc4ccc(OC)cc4C3)cc2C(F)(F)F)C1=O ; *N1Cc2c(cc(CN3CCC(c4ccccc4)C3)cc2C(F)(F)F)C1=O ; *N1Cc2c(cc(CN3CCOC(C)C3)cc2C(F)(F)F)C1=O ; *N1Cc2c(cc(CN3CC(C)OC(C)C3)cc2C(F)(F)F)C1=O]

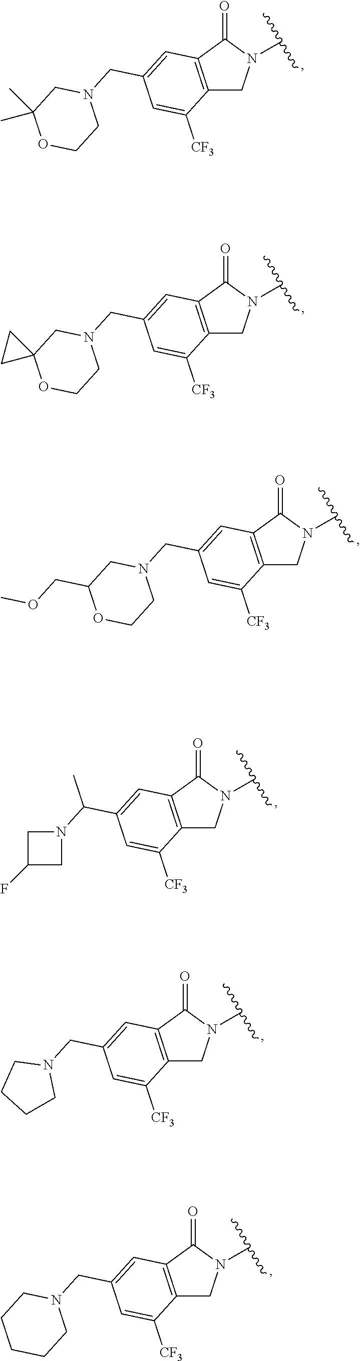 [SMILES: *N1Cc2c(cc(CN3CCOC(C)(C)C3)cc2C(F)(F)F)C1=O ; *N1Cc2c(cc(CN3CCOC4(CC4)C3)cc2C(F)(F)F)C1=O ; *N1Cc2c(cc(CN3CCOC(COC)C3)cc2C(F)(F)F)C1=O ; *N1Cc2c(cc(C(C)N3CC(F)C3)cc2C(F)(F)F)C1=O ; *N1Cc2c(cc(CN3CCCC3)cc2C(F)(F)F)C1=O ; *N1Cc2c(cc(CN3CCCCC3)cc2C(F)(F)F)C1=O]

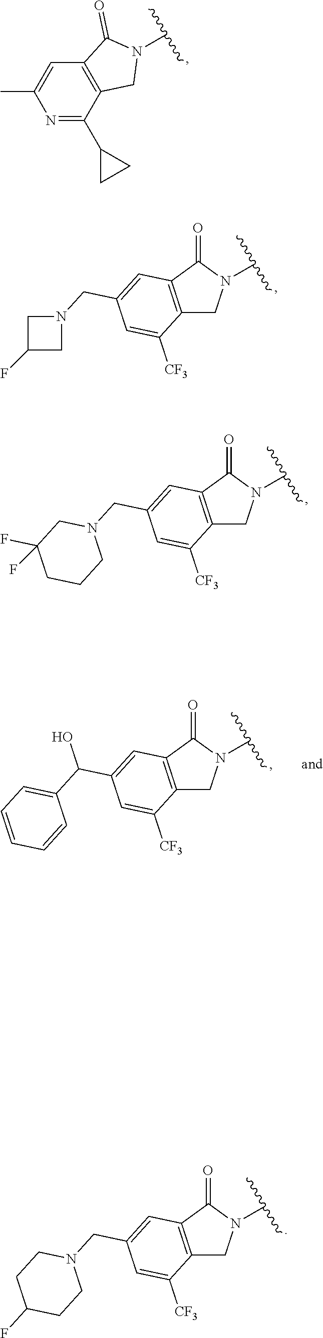 [SMILES: *N1Cc2c(cc(C)nc2C2CC2)C1=O ; *N1Cc2c(cc(CN3CC(F)C3)cc2C(F)(F)F)C1=O ; *N1Cc2c(cc(CN3CCCC(F)(F)C3)cc2C(F)(F)F)C1=O ; *N1Cc2c(cc(C(O)c3ccccc3)cc2C(F)(F)F)C1=O ; *N1Cc2c(cc(CN3CCC(F)CC3)cc2C(F)(F)F)C1=O]

168. The pharmaceutical composition of any one of claims 124-167, wherein ring C is selected from the group consisting of 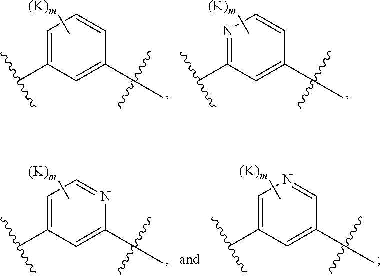
wherein K is independently selected from the group consisting of F, Cl, Br, I, —CN, C₁-C₈ alkyl optionally substituted with —OH or —O—C₁-C₈ alkyl, C₃-C₈ cycloalkyl optionally substituted with —OH or —O—C₁-C₈ alkyl, —O—C₁-C₈ alkyl, —O—C₁-C₈ haloalkyl, and —NR^{g}R^{h}, where R^{g} and R^{h} are independently selected from the group consisting of H, C₁-C₈ alkyl optionally substituted with —OH, C₃-C₈ cycloalkyl optionally substituted with —OH, and four- to eight-membered heterocyclyl, or where R^{g} and R^{h} are taken together with the nitrogen to which they are attached to form a four- to eight-membered heterocyclic ring optionally substituted with OH, F, Cl, Br, I, C₁-C₈ alkyl, C₁-C₈ haloalkyl, C₁-C₈ hydroxyalkyl, —CN, or —O—C₁-C₈ alkyl; and m is 0, 1, or 2.

169. The pharmaceutical composition of any one of claims 124-167, wherein ring C is 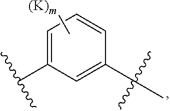
wherein m is 0, 1, or 2, and each K is independently selected from the group consisting of:
F, Cl, Br, I, —CN,
C₁-C₈ alkyl optionally substituted with —OH or —O—C₁-C₈ alkyl,
—O—C₁-C₈ alkyl,
—O—C₁-C₈ haloalkyl, and
—NR^{g}R^{h}, where R^{g} and R^{h} are independently selected from the group consisting of H, C₃-C₈ alkyl optionally substituted with —OH, and heterocyclyl.

170. The pharmaceutical composition of any one of claims 124-167, wherein ring C is 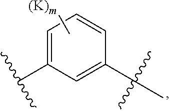
wherein m is 0, 1, or 2, and each K is independently:
C₃-C₈ cycloalkyl optionally substituted with —C₁-C₈ alkyl, —OH or —O—C₁-C₈ alkyl, or
—NR^{g}R^{h}, where R^{g} and R^{h} are independently selected from the group consisting of H, C₃-C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl.

171. The pharmaceutical composition of any one of claims 124-167, wherein ring C is selected from the group consisting of 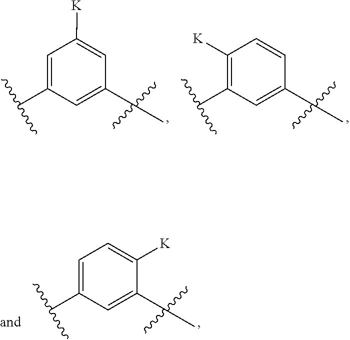
wherein K is selected from the group consisting of F, Cl, Br, I, —CN, C₁-C₈ alkyl optionally substituted with —OH or —O—C₁-C₈ alkyl, —O—C₁-C₈ alkyl, —O—C₁-C₈ haloalkyl, and —NR^{g}R^{h}, where R^{g} and R^{h} are independently selected from the group consisting of H, C₁-C₈ alkyl optionally substituted with —OH, and heterocyclyl.

172. The pharmaceutical composition of any one of claims 124-167, wherein ring C is selected from the group consisting of

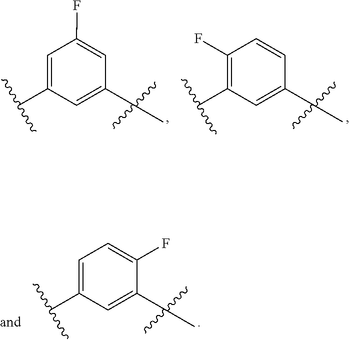 [SMILES: *c1cc(*)cc(F)c1 ; *c1ccc(F)c(*)c1 ; *c1ccc(F)c(*)c1]

173. The pharmaceutical composition of any one of claims 124-170, wherein two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

174. The pharmaceutical composition of any one of claims 124-170, wherein two vicinal K groups are taken together with the atoms to which they are attached to form a five-membered carbocyclic or heterocyclic ring, wherein the five-membered carbocyclic or heterocyclic ring are each optionally substituted with one or two substituents selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl.

175. The pharmaceutical composition of any one of claims 124-167, wherein ring C is selected from the group consisting of

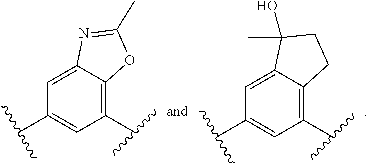 [SMILES: *c1cc(*)c2oc(C)nc2c1 ; *c1cc(*)c2c(c1)C(C)(O)CC2]

176. The pharmaceutical composition of any one of claims 124-175, wherein one of R⁷ and R⁸ is C₁-C₈ alkyl.

177. The pharmaceutical composition of any one of claims 124-175, wherein R⁷ is methyl.

178. The pharmaceutical composition of any one of claims 124-175, wherein R⁸ is methyl.

179. The pharmaceutical composition of any one of claims 124-175, wherein one of R⁷ and R⁸ is H, F, CF₃, or —CH₂OCH₃.

180. The pharmaceutical composition of any one of claims 124-175, wherein one of R⁷ and R⁸ is methyl and the other is H or F.

181. The pharmaceutical composition of any one of claims 124-175, wherein R⁷ and R⁸ together with the carbon to which they are attached form a C₃-C₈ cycloalkyl ring or three- to six-membered heterocyclyl ring, wherein the C₃-C₈ cycloalkyl ring or the three- to six-membered heterocyclyl ring is optionally substituted with F, Cl, Br, I, —OH, C₁-C₄ alkyl, or —O—C₁-C₄ alkyl.

182. The pharmaceutical composition of any one of claims 124-175, wherein R⁷ and R⁸ together with the carbon to which they are attached form a cyclopropyl or oxetanyl ring, wherein the cyclopropyl or oxetanyl ring is optionally substituted with F, Cl, Br, I, —OH, C₁-C₄ alkyl, or —O—C₁-C₄ alkyl.

183. The pharmaceutical composition of any one of claims 124-182, wherein Y is C(R⁹)(R¹⁰), wherein R⁹ is selected from the group consisting of H, F, Cl, Br, I, and C₁-C₈ alkyl, and R¹⁰ is selected from the group consisting of H, F, Cl, Br, I, and C₁-C₈ alkyl.

184. The pharmaceutical composition of claim 164, wherein Y is CH₂.

185. The pharmaceutical composition of any one of claims 124-182, wherein Y is S.

186. The pharmaceutical composition of any one of claims 124-175, wherein R⁹ is taken together with R⁸ to form a three- to six-membered cycloalkyl, heterocyclyl, aryl, or heteroaryl ring, each optionally substituted with F, Cl, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

187. The pharmaceutical composition of any one of claims 124-175, wherein R⁹ is taken together with R⁸ to form a three- to six-membered cycloalkyl or heterocyclyl ring, wherein the three- to six-membered cycloalkyl or heterocyclyl ring are each optionally substituted with F, C₁, Br, I, —OH, C₁-C₈ alkyl optionally substituted with —OH or —O—C₁-C₈ alkyl, or —O—C₁-C₈ alkyl optionally substituted with —OH or —O—C₁-C₈ alkyl.

188. The pharmaceutical composition of any one of claims 124-175, wherein R⁹ is taken together with R⁸ to form a cyclopropyl, cyclobutyl, cyclopentyl, cyclohexyl, aziridinyl, oxiranyl, thiiranyl, azetidinyl, oxetanyl, thietanyl, pyrrolidinyl, pyrazolidinyl, imidazolidinyl, tetrahydrofuranyl, 1,3-dioxolanyl, tetrahydrothiophenyl, oxathiolanyl, sulfolanyl, piperidinyl, piperazinyl, tetrahydropyranyl, dioxanyl, thianyl, dithianyl, trithianyl, morpholinyl, or thiomorpholinyl ring.

189. The pharmaceutical composition of any one of claims 124-184 or 186-187, wherein Y is C(R⁹)(R¹⁰), and the absolute configuration of the carbon atom to which R⁷ and R⁸ are attached is (R).

190. The pharmaceutical composition of any one of claims 124-184 or 186-187, wherein Y is C(R⁹)(R¹⁰), and the absolute configuration of the carbon atom to which R⁷ and R⁸ are attached is (S).

191. The pharmaceutical composition of any one of claims 124-182 or 185, wherein Y is S, and the absolute configuration of the carbon atom to which R⁷ and R⁸ are attached is (S).

192. The pharmaceutical composition of any one of claims 124-182 or 185, wherein Y is S, and the absolute configuration of the carbon atom to which R⁷ and R⁸ are attached is (R).

193. The pharmaceutical composition of claim 124, wherein [symbol] is a double bond, Y is C(R⁹); and Z is C(R⁸), wherein R⁸ is taken together with R⁹ to form a three- to six-membered cycloalkyl, heterocyclyl, aryl, or heteroaryl ring, each optionally substituted with F, Cl, Br, I, —OH, C₁-C₄ alkyl optionally substituted with —OH or —O—C₁-C₈ alkyl, or —O—C₁-C₄ alkyl optionally substituted with —OH or —O—C₁-C₈ alkyl.

194. The pharmaceutical composition of any one of claims 124-193, wherein Ring B is a five-membered heteroaryl ring containing at least one N, O, or S ring atom, wherein Ring B is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

195. The pharmaceutical composition of any one of claims 124-193, wherein Ring B is a five-membered heteroaryl ring containing at least one N, O, or S ring atom, wherein Ring B is optionally substituted with one, two, or three substituents independently selected from the group consisting of F, Cl, Br, and I.

196. The pharmaceutical composition of any one of claims 124-193, wherein Ring B is a five-membered heteroaryl ring containing at least one N, O, or S ring atom, wherein Ring B is optionally substituted with one, two, or three substituents independently selected from the group consisting of methyl, ethyl, cyclopropyl, and —CH₂OH.

197. The pharmaceutical composition of any one of claims 124-193, wherein Ring B is selected from the group consisting of pyrrole, imidazole, 1,2,4-triazole, 1,2,3-triazole, pyrazole, tetrazole, oxadiazole, oxazole, and isoxazole, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

198. The pharmaceutical composition of any one of claims 124-193, wherein Ring B is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

199. The pharmaceutical composition of any one of claims 124-193, wherein Ring B is selected from the group consisting of pyrrol-2-yl, pyrrol-3-yl, imidazol-2-yl, imidazol-4-yl, imidazol-5-yl, 1,2,4-triazol-3-yl, 1,2,3-triazol-4-yl, pyrazol-3-yl, pyrazol-4-yl, tetrazol-5-yl, 1,3,4-oxadiazol-2-yl, oxazol-3-yl, and isoxazol-3-yl, each of which is optionally substituted with methyl, ethyl, cyclopropyl, or —CH₂OH.

200. The pharmaceutical composition of any one of claims 124-193, wherein Ring B is selected from the group consisting of

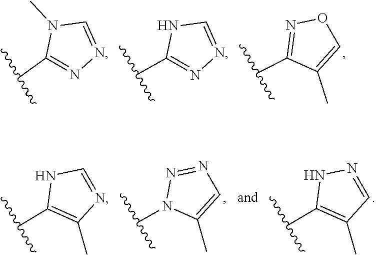

201. The pharmaceutical composition of any one of claims 124-193, wherein Ring B is selected from the group consisting of

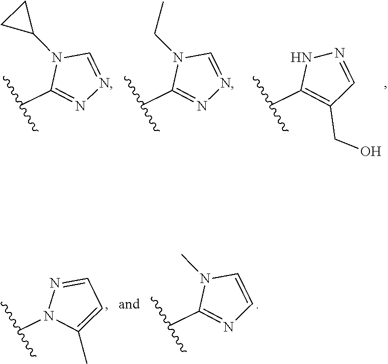

202. The pharmaceutical composition of any one of claims 124-193, wherein Ring B is selected from the group consisting of

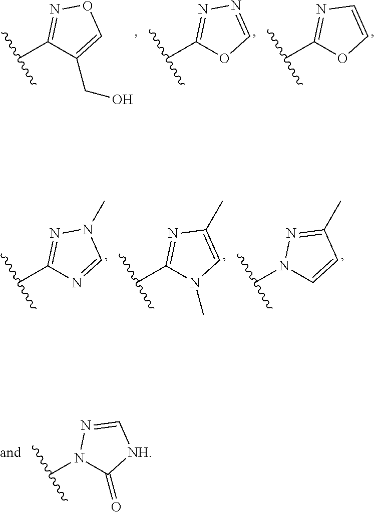

203. The pharmaceutical composition of any one of claims 124-193, wherein Ring B is selected from the group consisting of

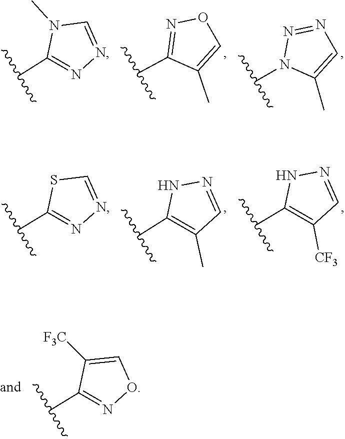

204. The pharmaceutical composition of any one of claims 124-193, wherein Ring B is 4-methyl-4H-1,2,4-triazol-3-yl.

205. The pharmaceutical composition of any one of claims 124-204, wherein the pharmaceutical composition is sterile.

206. The pharmaceutical composition of any one of claims 124-205, wherein the composition is suitable for intravenous, intraarterial, intramuscular, peritoneal, intrathecal, or subcutaneous injection.

207. The pharmaceutical composition of any one of claims 124-206, wherein the pharmaceutically acceptable excipient is selected from the group consisting of water, saline, Ringer's solution, or isotonic sodium chloride solution.

208. The pharmaceutical composition of any one of claims 124-207, with the proviso that the compounds of Formula (III-A) or Formula (III) excludes compounds of Table IX, tautomers of the compounds of Table IX, or salts of the compounds or tautomers.

209. The pharmaceutical composition of claim 124 wherein the compound of Formula (III-A) or Formula (III) is selected from the group consisting of the compounds of Table 1, including “a” and “b” variants of the compounds, tautomers thereof, and salts of the compounds or tautomers.

210. The pharmaceutical composition of claim 127, wherein the compound is selected from Compounds 1-7, 26-29, 31-47, 49-61, 64-77, 79-82, 84-85, 87-94, 95, 95a, 95b, 102a-106, 110, 113-114, 116-119, 122-127, 130a, 133, 138-161, 170, 181, 183a-195, 197-208, 212-254, 284-288, or 302 of Table 1, including “a” and “b” variants of the compounds, tautomers thereof, and pharmaceutically acceptable salts of the compounds or tautomers.

211. The pharmaceutical composition of claim 124, wherein the compound of Formula (III-A) or Formula (III) is selected from the group consisting of the compounds 8a, 57a, 140, 255a, 183a, 282, and 187, tautomers thereof, and pharmaceutically acceptable salts of the compounds or tautomers.

212. The pharmaceutical composition of claim 124, wherein the compound of Formula (III-A) or Formula (III) is selected from the group consisting of the compounds 8a, 57a, 140, 183a, and 187, tautomers thereof, and pharmaceutically acceptable salts of the compounds or tautomers.

213. The pharmaceutical composition of claim 124, wherein the compound of Formula (III-A) or Formula (III) is selected from the group consisting of the compounds 255a and 282, tautomers thereof, and pharmaceutically acceptable salts of the compounds or tautomers.

214. The pharmaceutical composition of claim 124, wherein the compound of Formula (III-A) or Formula (III) is selected from the group consisting of

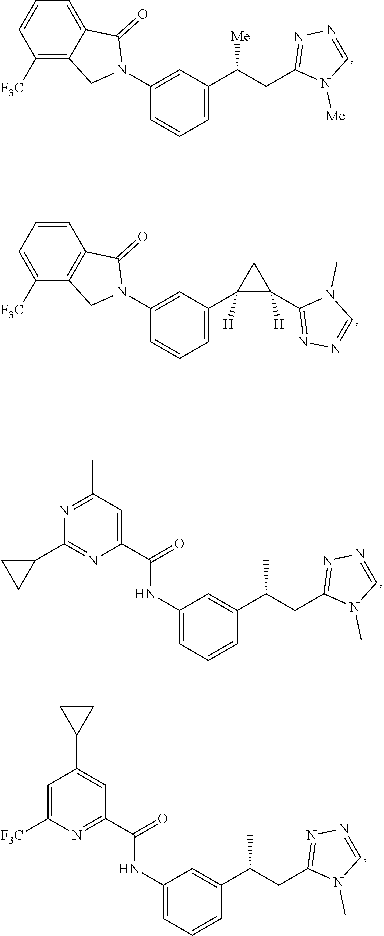 [SMILES: C[C@H](Cc1nncn1C)c1cccc(N2Cc3c(cccc3C(F)(F)F)C2=O)c1 ; Cn1cnnc1[C@@H]1C[C@@H]1c1cccc(N2Cc3c(cccc3C(F)(F)F)C2=O)c1 ; Cc1cc(C(=O)Nc2cccc([C@H](C)Cc3nncn3C)c2)nc(C2CC2)n1 ; C[C@H](Cc1nncn1C)c1cccc(NC(=O)c2cc(C3CC3)cc(C(F)(F)F)n2)c1]

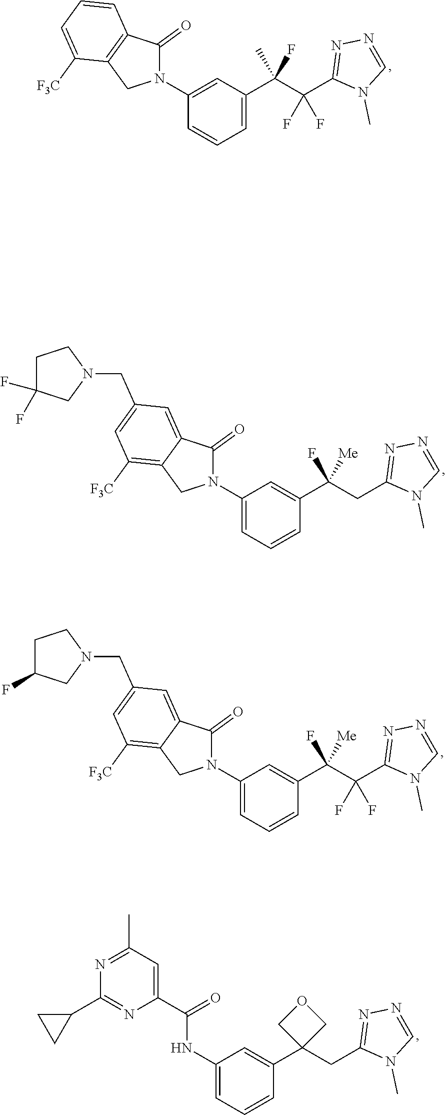 [SMILES: Cn1cnnc1C(F)(F)[C@](C)(F)c1cccc(N2Cc3c(cccc3C(F)(F)F)C2=O)c1 ; Cn1cnnc1C[C@](C)(F)c1cccc(N2Cc3c(cc(CN4CCC(F)(F)C4)cc3C(F)(F)F)C2=O)c1 ; Cn1cnnc1C(F)(F)[C@](C)(F)c1cccc(N2Cc3c(cc(CN4CC[C@H](F)C4)cc3C(F)(F)F)C2=O)c1 ; Cc1cc(C(=O)Nc2cccc(C3(Cc4nncn4C)COC3)c2)nc(C2CC2)n1]

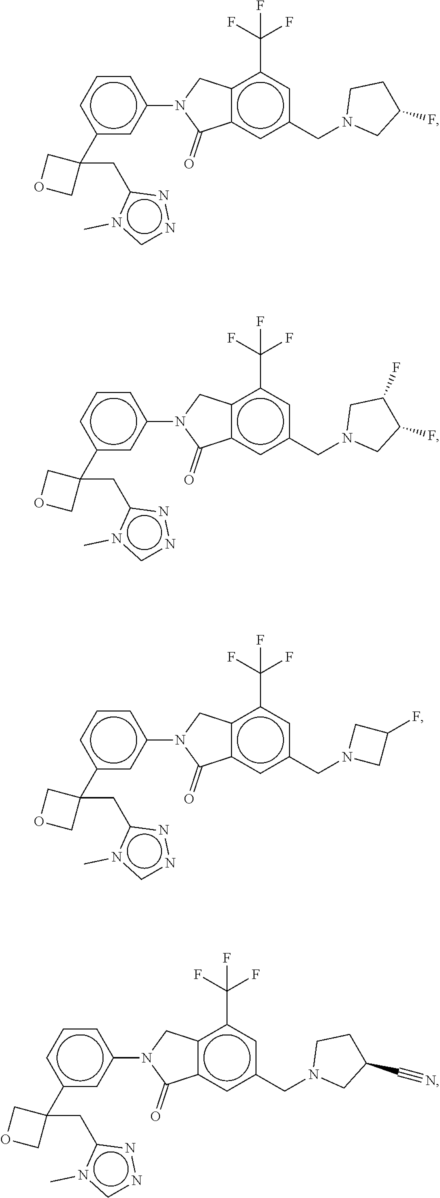

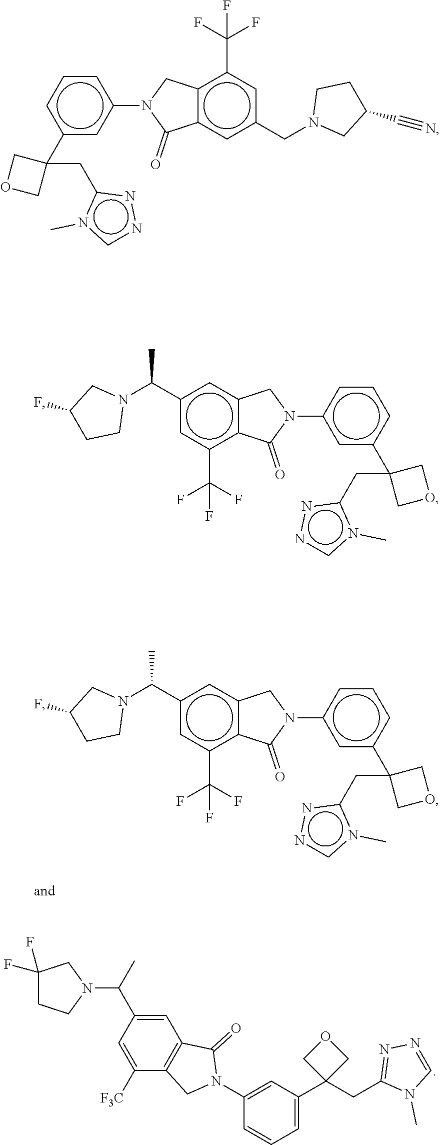

215. A compound of Formula (IV): 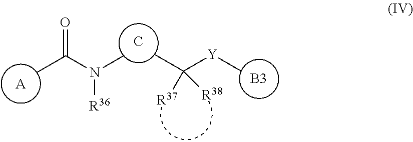
or a tautomer thereof, or a pharmaceutically acceptable salt of any of the foregoing,
wherein Ring A, 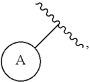
is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;
wherein Ring A is attached to the adjacent carbonyl carbon via a carbon atom of Ring A;
each R^{A} is independently selected from the group consisting of:
H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —C₁-C₈ haloalkyl-OH, —C₁-C₈ haloalkyl-COOH, —CO(C₁-C₈ alkyl), —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,
—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five-to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to nine-membered heterocyclic ring, a five- to eight-membered heteroaryl ring, a —(C₁-C₄ alkylene)-(four- to ten-membered heterocyclic ring), a —CH(C₁-C₈ alkyl) (four- to eight-membered heterocyclic ring), a —CH(C₁-C₈ haloalkyl)-(four- to eight-membered heterocyclic ring), a —CH(OH)-(C₆-C₁₄ aryl ring), a —C(O)-(five- to eight-membered heterocyclic ring), a —O-(four- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C₁-C₈ alkyl-CN, —C₁-C₈ alkyl-OH, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring,
(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three-to eight-membered heterocyclic ring where the carbocyclic or heterocyclic ring is optionally substituted with C₁-C₄ alkyl or OH;
—S(═O)₂—C₁-C₈ alkyl,
—SF₅, and
—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five-to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
R³⁶ is H, or
R³⁶ and the amide group to which R³⁶ is connected attach to Ring A to form a five-membered lactam ring fused to Ring A, such that the fragment 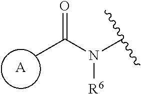
is 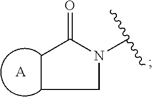
Ring C, 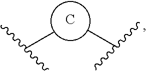
is selected from the group consisting of: 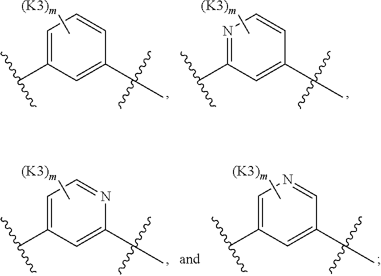
each K3 is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —C₁-C₈ alkyl, —OH or —O—C₁-C₈ alkyl,
—O—C₁-C₈ alkyl optionally substituted with —OH,
—O—C₁-C₈ haloalkyl,
—O-(three- to six-membered heterocyclic ring) optionally substituted with C₁-C₈ alkyl,
a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, Cr C₈ alkyl optionally substituted with —OH, C₃-C₈ cycloalkyl optionally substituted with —OH or C₁-C₄ alkyl, four- to eight-membered heterocyclyl optionally substituted with —OH or C₁-C₄ alkyl, —CO—(C₁-C₈ haloalkyl), —CO—(three- to six-membered heterocyclic ring), and —SO₂—C₂-C₈ alkenyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K groups are taken together with the atoms to which they are attached to form a three- to six-membered carbocyclic or heterocyclic ring, a phenyl ring, or a five- to six-membered heteroaryl ring, wherein the carbocyclic or heterocyclic ring, the phenyl ring, or the heteroaryl ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;
m is 0, 1, or 2;
R³⁷ and R³⁸ together with the carbon to which they are attached form a C₃-C₈ cycloalkyl ring or three- to six-membered heterocyclyl ring as indicated by the dashed curve [symbol] , wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;
Y is C(R³⁹)(R⁴⁰) or S;
R³⁹ and R⁴⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; or
R³⁹ and R⁴⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;
and
Ring B3, 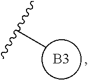
is a five-membered heteroaryl ring containing at least one N, O, or S ring atom, wherein Ring B3 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

216. The compound of claim 215, wherein
Ring A, 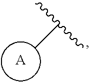
is selected from the group consisting of C₃-C₈ cycloalkyl optionally substituted with one, two, or three independently chosen R^{A} groups, C₆-C₁₀ aryl optionally substituted with one, two, or three independently chosen R^{A} groups, a five- to ten-membered heterocyclic ring system optionally substituted with one, two, or three independently chosen R^{A} groups, and a five- to ten-membered heteroaryl ring system optionally substituted with one, two, or three independently chosen R^{A} groups;
wherein Ring A is attached to the adjacent carbonyl carbon via a carbon atom of Ring A;
each R^{A} is independently selected from the group consisting of:
H, F, Cl, Br, I, —CN, —OH, oxo, C₁-C₈ alkyl, C₃-C₈ cycloalkyl, —O—C₁-C₈ alkyl, —C₁-C₈ alkyl-OH, C₁-C₈ haloalkyl, —O—C₁-C₈ haloalkyl, —O—C₁-C₈ haloalkyl-OH, —COOH, —CONH₂, —C₁-C₈ alkylene-COOH, —C₁-C₈ alkylene-CONH₂, —O—C₁-C₈ alkylene-COOH, —O—C₁-C₈ alkylene-CONH₂, —C₁-C₈ alkylene-heterocyclyl, and —O—C₁-C₈ alkylene-heterocyclyl,
—O—C₃-C₈ cycloalkyl optionally substituted with one, two, or three moieties independently selected from the group consisting of —OH, —C₁-C₈ alkyl, —O—C₁-C₈ alkyl, C₁-C₈ haloalkyl, —O—C₁-C₈ haloalkyl, F, Cl, Br, I, —CN, and —NR^{B}R^{C}, where R^{B} and R^{c} are independently H, C₁-C₈ alkyl, or C₁-C₈ haloalkyl,
—NR^{a}R^{b} where R^{a} and R^{b} are independently H, C₁-C₈ alkyl, C₁-C₈ haloalkyl, C₃-C₈ cycloalkyl, or a three- to eight-membered heterocyclic ring, where the alkyl or cycloalkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); where R^{b} can additionally be chosen from —C(═O)—C₁-C₈ alkyl or —S(═O)₂—C₁-C₈ alkyl where the alkyl groups are optionally substituted with —OH, —O—C₁-C₄ alkyl, —CN, F, Cl, Br, I, —NH₂, —NH(C₁-C₄ alkyl), or —N(C₁-C₄ alkyl)(C₁-C₄ alkyl); or R^{a} and R^{b} are taken together with the nitrogen to which they are attached to form a four- to eight-membered heterocyclic ring or a five-to eight-membered heteroaryl ring, wherein the heterocyclic ring or heteroaryl ring is optionally substituted with —OH, —CN, oxo, F, Cl, Br, I, C₁-C₈ alkyl, —O—C₁-C₈ alkyl, C₁-C₈ haloalkyl, —C(═O)—C₁-C₈ alkyl, or —O—C₁-C₈ haloalkyl,
a three- to eight-membered heterocyclic ring, a five- to eight-membered heteroaryl ring, a —(C₁-C₄ alkylene)-(four- to eight-membered heterocyclic ring), a —CH(CH₃)-(four- to eight-membered heterocyclic ring), a —C(O)-(five- to eight-membered heterocyclic ring), a —O—(C₁-C₄ alkylene)-(five- to eight-membered heterocyclic ring), a —(C₁-C₄ alkylene)-(five- to eight-membered heteroaryl ring), or a —O—(C₁-C₄ alkylene)-(five- to eight-membered heteroaryl ring), wherein the heterocyclic or heteroaryl ring contains an S(═O)₂ group or one, two, or three heteroatoms independently selected from the group consisting of O, N, and S, and wherein the heterocyclic or heteroaryl ring is optionally substituted with one, two, or three substituents independently selected from the group consisting of —OH, oxo, C₁-C₈ alkyl, —O—C₁-C₈ alkyl, C₁-C₈ haloalkyl, —O—C₁-C₈ haloalkyl, —C(═O)—C₁-C₈ alkyl, F, Cl, Br, I, —CN, —C(═O)OH, and —S(═O)₂—C₁-C₈ alkyl, or wherein the heterocyclic or heteroaryl ring is optionally fused to a spiro three-to-six membered carbocyclic ring or a spiro three-to-six membered heteroaryl ring,
(C₁-C₄ alkylene)-NR^{c}R^{d}, —O—(C₁-C₄ alkylene)-NR^{c}R^{d}, —C(═O)NR^{c}R^{d}, —(C₁-C₄ alkylene)-C(═O)NR^{c}R^{d}, or —O—(C₁-C₄ alkylene)-C(═O)NR^{c}R^{d}, wherein R^{c} and R^{d} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{c} and R^{d} are taken together with the nitrogen to which they are attached to form a three- to eight-membered heterocyclic ring or a five- to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three-to eight-membered heterocyclic ring where the carbocyclic or heterocyclic ring is optionally substituted with C₁-C₄ alkyl or OH;
—S(═O)₂—C₁-C₈ alkyl,
—SF₅, and
—S(═O)₂NR^{e}R^{f} wherein R^{e} and R^{f} are independently H, C₁-C₈ alkyl, C₃-C₈ cycloalkyl, or four- to eight-membered heterocyclyl, where the alkyl, cycloalkyl, and heterocyclyl groups are optionally substituted with —OH, F, Cl, Br, I, —NH₂, —NH(C₁-C₄ alkyl), or —N(C₁-C₄ alkyl)(C₁-C₄ alkyl); or R^{e} and R^{f} are taken together with the nitrogen to which they are attached to form a three- to eight-membered heterocyclic ring or a five-to eight-membered heteroaryl ring, wherein the heterocyclic ring or heteroaryl ring is optionally substituted with one, two, or three substituents independently selected from the group consisting of —OH, oxo, —CN, C₁-C₄ alkyl, —O—C₁-C₄ alkyl, C₁-C₄ haloalkyl, —O—C₁-C₄ haloalkyl, —C₁-C₄ alkyl-OH, —C(═O)—C₁-C₄ alkyl, —O—C₁-C₈ alkyl, F, Cl, Br, I, —S(═O)₂—C₁-C₈ alkyl, —NH₂, —NH(C₁-C₄ alkyl), or —N(C₁-C₄ alkyl)(C₁-C₄ alkyl), where two substituents on the heterocyclic ring or heteroaryl ring can be taken together to form a three- to eight-membered carbocyclic ring or a three- to eight-membered heterocyclic ring where the carbocyclic or heterocyclic ring is optionally substituted with C₁-C₄ alkyl or OH;
R³⁶ is H, or
R³⁶ and the amide group to which R³⁶ is connected attach to Ring A to form a five-membered lactam ring fused to Ring A, such that the fragment 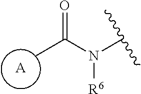
is 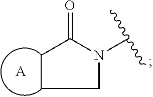
Ring C, 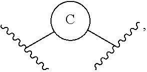
is selected from the group consisting of: 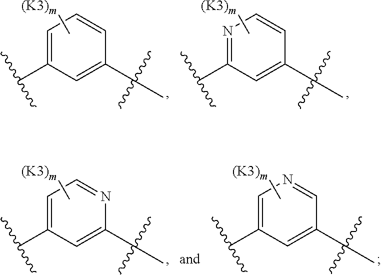
each K3 is independently selected from the group consisting of:
F, Cl, Br, I, —CN, —OH,
C₁-C₈ alkyl optionally substituted with —OH or —O—C₁-C₈ alkyl,
C₃-C₈ cycloalkyl optionally substituted with —OH or —O—C₁-C₈ alkyl,
—O—C₁-C₈ alkyl optionally substituted with —OH,
—O—C₁-C₈ haloalkyl,
—O-(three- to six-membered heterocyclic ring) optionally substituted with C₁-C₈ alkyl, a three- to six-membered carbocyclic ring, a three- to six-membered heterocyclic ring, a phenyl ring, a five- to six-membered heteroaryl ring, where the carbocyclic, heterocyclic, phenyl, or heteroaryl ring is optionally substituted by one or two substituents independently selected from the group consisting of F, Cl, Br, I, —CN, —OH, C₁-C₈ alkyl optionally substituted with —OH or —O—C₁-C₈ alkyl, and —O—C₁-C₈ alkyl; and
—NR^{g}R^{h}, where R^{g} and R^{h} are independently selected from the group consisting of H, C₁-C₈ alkyl optionally substituted with —OH, C₃-C₈ cycloalkyl optionally substituted with —OH or C₁-C₄ alkyl, and four- to eight-membered heterocyclyl optionally substituted with —OH or C₁-C₄ alkyl, or where R^{g} and R^{h} are taken together with the nitrogen to which they are attached to form a four- to eight-membered heterocyclic ring optionally substituted with —OH, F, Cl, Br, I, C₁-C₈ alkyl, C₁-C₈ haloalkyl, C₁-C₈ hydroxyalkyl, —CN, or —O—C₁-C₈ alkyl;
or two vicinal K groups are taken together with the atoms to which they are attached to form a three- to six-membered carbocyclic or heterocyclic ring, a phenyl ring, or a five- to six-membered heteroaryl ring, wherein the ring formed by the two vicinal K groups is optionally substituted by one or two substituents independently selected from the group consisting of F, Cl, Br, I, —CN, —OH, C₁-C₈ alkyl optionally substituted with —OH or —O—C₁-C₈ alkyl, —O—C₁-C₈ alkyl, and —NR^{g1}R^{h1}, where R^{g1} and R^{h1} are independently H or C₁-C₈ alkyl;
m is 0, 1, or 2;
R³⁷ and R³⁸ together with the carbon to which they are attached form a C₃-C₈ cycloalkyl ring or three- to six-membered heterocyclyl ring as indicated by the dashed curve [symbol] , wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH;
Y is C(R³⁹)(R⁴⁰) or S;
R³⁹ and R⁴⁰ are independently selected from the group consisting of H, F, Cl, Br, I, —OH, C₁-C₈ alkyl optionally substituted with —OH or —O—C₁-C₈ alkyl, and —O—C₁-C₈ alkyl optionally substituted with —OH or —O—C₁-C₈ alkyl; or
R³⁹ and R⁴⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, —O—C₁-C₄ alkyl, or —C₁-C₈ alkylene-OH; and
Ring B3, 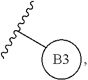
is a five-membered heteroaryl ring containing at least one N, O, or S ring atom, wherein Ring B3 is optionally substituted with one, two, or three substituents independently selected from the group consisting of F, Cl, Br, I, C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

217. The compound or tautomer thereof, or pharmaceutically acceptable salt of the compound or tautomer, of claim 215 or claim 216, wherein R³⁷ and R³⁸ together with the carbon to which they are attached combine to form an oxetane ring.

218. The compound or tautomer thereof, or pharmaceutically acceptable salt of the compound or tautomer, of claim 215 or claim 216, wherein R³⁷ and R³⁸ together with the carbon to which they are attached combine to form a cyclobutyl ring.

219. The compound or tautomer thereof, or pharmaceutically acceptable salt of the compound or tautomer, of claim 217, wherein the compound is of Formula (IV-ox): 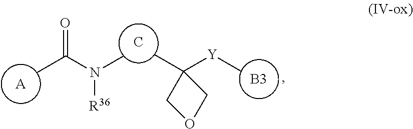
or a tautomer thereof, or a pharmaceutically acceptable salt of any of the foregoing.

220. The compound or tautomer thereof, or pharmaceutically acceptable salt of the compound or tautomer, of any one of claims 215-219, wherein Ring B3 is selected from the group consisting of pyrrole, imidazole, 1,2,4-triazole, 1,2,3-triazole, pyrazole, tetrazole, oxadiazole, oxazole, and isoxazole, each of which is optionally substituted with one, two, or three substituents independently selected from the group consisting of C₁-C₈ alkyl, —C₁-C₈ alkyl-OH, C₃-C₈ cycloalkyl, —O—C₁-C₈ alkyl, C₁-C₈ haloalkyl, and —O—C₁-C₈ haloalkyl.

221. The compound or tautomer thereof, or pharmaceutically acceptable salt of the compound or tautomer, of any one of claims 215-219, wherein Ring B3 is 4-methyl-4H-1,2,4-triazol-3-yl.

222. The compound or tautomer thereof, or pharmaceutically acceptable salt of the compound or tautomer, of any one of claims 215-221, wherein Y is C(R³⁹)(R⁴⁰).

223. The compound or tautomer thereof, or pharmaceutically acceptable salt of the compound or tautomer, of claim 222, wherein R³⁹ is H and R⁴⁰ is H.

224. The compound or tautomer thereof, or pharmaceutically acceptable salt of the compound or tautomer, of claim 222, wherein at least one of R³⁹ and R⁴⁰ is F.

225. The compound or tautomer thereof, or pharmaceutically acceptable salt of the compound or tautomer, of claim 222, wherein R³⁹ is H and R⁴⁰ is F.

226. The compound or tautomer thereof, or pharmaceutically acceptable salt of the compound or tautomer, of claim 222, wherein R³⁹ is F and R⁴⁰ is F.

227. The compound or tautomer thereof, or pharmaceutically acceptable salt of the compound or tautomer, of claim 222, wherein R³⁹ and R⁴⁰ together with the carbon to which they are attached form a C₃-C₈ cycloalkyl ring or three- to six-membered heterocyclyl ring, wherein the cycloalkyl or heterocyclyl ring is optionally substituted with F, Cl, Br, I, —OH, C₁-C₄ alkyl, or —O—C₁-C₄ alkyl.

228. The compound or tautomer thereof, or pharmaceutically acceptable salt of the compound or tautomer, of claim 227, wherein R³⁹ and R⁴⁰ together with the carbon to which they are attached form a C₃ cycloalkyl ring optionally substituted with F, Cl, Br, I, —OH, C₁-C₄ alkyl, or —O—C₁-C₄ alkyl.

229. The compound or tautomer thereof, or pharmaceutically acceptable salt of the compound or tautomer, of any one of claims 215-228, wherein Ring C is

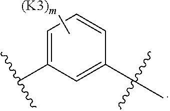

230. The compound or tautomer thereof, or pharmaceutically acceptable salt of the compound or tautomer, of claim 229, wherein m is 0.

231. The compound or tautomer thereof, or pharmaceutically acceptable salt of the compound or tautomer, of any one of claims 215-230, or the composition of any one of claims 124-214, wherein Ring A is phenyl, pyridyl, or pyrimidyl.

232. The compound or tautomer thereof, or pharmaceutically acceptable salt of the compound or tautomer, of any one of claims 215-231, or the composition of any one of embodiments 124-214, wherein Ring A is pyrimidyl.

233. The compound or tautomer thereof, or pharmaceutically acceptable salt of the compound or tautomer, of any one of claims 215-232, or the composition of any one of claims 124-214, wherein the Ring A is substituted with one or more F, —CF₃, or cyclopropyl groups.

234. The compound or tautomer thereof, or pharmaceutically acceptable salt of the compound or tautomer, of any one of claims 215-230, or the composition of any one of embodiments 124-214, wherein Ring A is phenyl, pyridyl, or pyrimidyl and is substituted with one, two, or three C₁-C₈ alkyl, C₁-C₈ haloalkyl, C₃-C₈ cycloalkyl, CH₃, CF₃, or cyclopropyl groups.

235. The compound of any one of claims 215-230, or tautomer thereof, or pharmaceutically acceptable salt of the compound or tautomer, or the composition of any one of claims 124-214, wherein Ring A is phenyl, pyridyl, or pyrimidyl and is substituted with one or more F, —CF₃, or cyclopropyl groups.

236. The compound or tautomer thereof, or pharmaceutically acceptable salt of the compound or tautomer, of claim 215 or claim 216, wherein the compound is selected from the group consisting

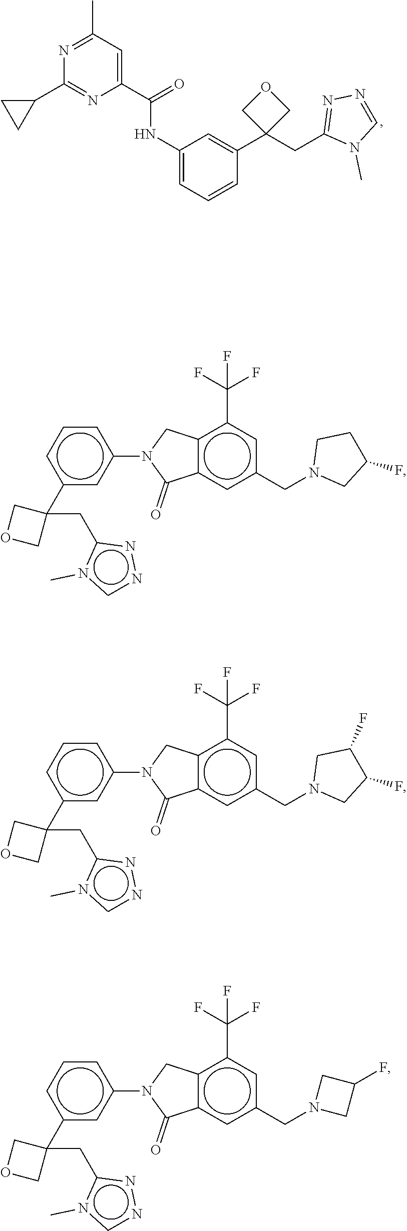

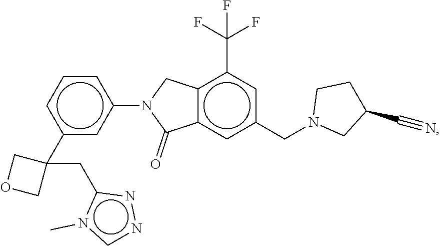

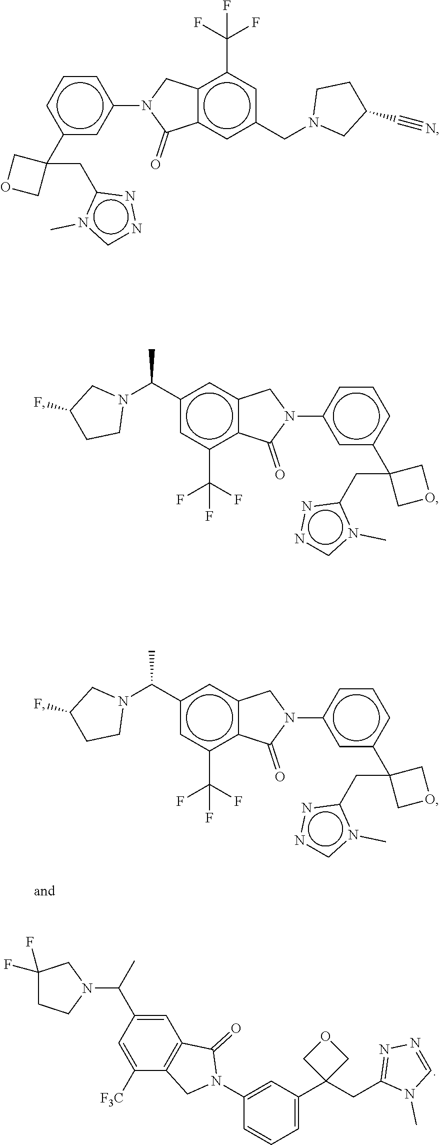

237. The compound or tautomer thereof, or pharmaceutically acceptable salt of the compound or tautomer, of any one of claims 1-123 and 215-236, wherein said compound, tautomer, or salt has a Cbl-b inhibition IC₅₀ of about 1 micromolar or less; or a Cbl-b inhibition IC₅₀ of about 300 nanomolar or less.

238. The compound or tautomer thereof, or pharmaceutically acceptable salt of the compound or tautomer, of any one of claims 1-123 and 215-236, wherein said compound, tautomer, or salt has a Cbl-b binding K_{D} of about 1 micromolar or less; or a Cbl-b binding K_{D} of about 300 nanomolar or less.

239. A method of modulating activity of an immune cell, the method comprising contacting the immune cell with an effective amount of a Cbl-b inhibitor to modulate activity of the immune cell, wherein the Cbl-b inhibitor is a compound of Formula (I-A), Formula (I), Formula (II-A), Formula (II), Formula (III), Formula (III-A), or Formula (IV) of any one of claims 1-238.

240. The method of claim 239, wherein the immune cell comprises a T-cell, a B cell, or a natural killer (NK) cell.

241. The method of claim 239 or claim 240, wherein the immune cell is a tumor-infiltrating lymphocyte (TIL) isolated from a tumor of a mammalian subject with cancer before the immune cell is contacted with the Cbl-b inhibitor.

242. The method of any one of claims 239-241, further comprising isolating the immune cell from a tumor of a mammalian subject with cancer before the immune cell is contacted with the Cbl-b inhibitor.

243. The method of any one of claims 239-242, wherein the immune cell comprises a T-cell, and wherein modulating activity of the T-cell comprises one or more of increased T-cell activation, increased T-cell proliferation, decreased T-cell exhaustion, and decreased T-cell tolerance.

244. The method of claim 243, wherein increased T-cell activation comprises increased production of a cytokine.

245. The method of claim 244, wherein the cytokine comprises one or more selected from the group consisting of IL-2, IFN-γ, TNFα, and GM-CSF.

246. The method of any one of claims 243-244, wherein increased T-cell activation comprises increased cell surface expression of one or more T-cell activation markers.

247. The method of claim 246, wherein the T-cell activation markers comprise one or more selected from the group consisting of CD25, CD69, and CTLA4.

248. The method of any one of claims 243-247, wherein the T-cell has been or is in contact with an anti-CD3 antibody alone or in combination with an anti-CD28 antibody.

249. The method of any one of claims 243-247, further comprising culturing the immune cell with IL-2 alone or in combination with an anti-CD3 antibody and/or an anti-CD28 antibody.

250. The method of any one of claims 239-242, wherein the immune cell comprises a NK cell, and wherein modulating activity of an NK cell comprises increased NK cell activation.

251. The method of claim 250, wherein increased NK cell activation comprises increased production of a cytokine.

252. The method of claim 251, wherein the cytokine comprises one or more selected from the group consisting of IFN-γ, TNFα, and MIP1β.

253. The method of any one of claims 239-252, wherein the immune cell comprises a B cell, and wherein modulating activity of a B cell comprises increased B cell activation, optionally wherein increased B cell activation comprises increased expression of CD69.

254. The method of any one of claims 239-253, wherein the immune cell is a human immune cell.

255. The method of any one of claims 239-254, wherein the immune cell comprises a recombinant chimeric receptor.

256. The method of claim 255, wherein the recombinant chimeric receptor is a chimeric antigen receptor.

257. A method of producing a modified immune cell, comprising culturing a cell population containing an immune cell in the presence of an effective amount of a Cbl-b inhibitor to modulate activity of the immune cell, thereby producing the modified immune cell, wherein the Cbl-b inhibitor is a compound of Formula (I-A), Formula (I), Formula (II-A), Formula (II), Formula (III), Formula (III-A), or Formula (IV) of any one of claims 1-238.

258. The method of claim 257, further comprising culturing the immune cell with an anti-CD3 antibody alone or in combination with an anti-CD28 antibody.

259. The method of claim 257, further comprising culturing of the immune cell with IL-2 alone or in combination with an anti-CD3 antibody and/or an anti-CD28 antibody.

260. The method of any one of claims 257-259, further comprising recovering the modified immune cell.

261. The method of any one of claims 257-260, wherein the immune cell is a tumor-infiltrating lymphocyte (TIL).

262. The method of any one of claims 257-260, wherein the immune cell is a cell selected from the group consisting of: a hematopoietic cell, a multipotent stem cell, a myeloid progenitor cell, a lymphoid progenitor cell, a T-cell, a B cell, and a NK cell.

263. The method of any one of claims 257-260, wherein the modified immune cell is a cell selected from the group consisting of: a hematopoietic cell, a multipotent stem cell, a myeloid progenitor cell, a lymphoid progenitor cell, a T-cell, a B cell, and a NK cell.

264. The method of any one of claims 257-263, wherein the immune cell is from an individual.

265. The method of any one of claims 257-264, wherein the immune cell is a human immune cell.

266. The method of any one of claims 257-265, wherein the immune cell or modified immune cell comprises a recombinant chimeric receptor.

267. The method of claim 266, wherein the recombinant chimeric receptor is a chimeric antigen receptor.

268. A modified immune cell produced by the method of any one of claims 257-267.

269. A modified immune cell comprising a Cbl-b inhibitor, wherein the Cbl-b inhibitor is a compound of Formula (I-A), Formula (I), Formula (II-A), Formula (II), Formula (III), Formula (III-A), or Formula (IV) of any one of claims 1-238.

270. An isolated modified immune cell, wherein the immune cell has been contacted or is in contact with a Cbl-b inhibitor, wherein the Cbl-b inhibitor is a compound of Formula (I-A), Formula (I), Formula (II-A), Formula (II), Formula (III), Formula (III-A), or Formula (IV) of any one of claims 1-238.

271. The modified immune cell of claim 270, wherein the modified immune cell is a T-cell, a B cell or a NK cell.

272. The modified immune cell of claim 270 or claim 271, wherein the immune cell is a tumor-infiltrating lymphocyte (TIL) isolated from a tumor of a mammalian subject with cancer before the immune cell is contacted with the Cbl-b inhibitor.

273. The modified immune cell of any one of claims 270-272, wherein the modified immune cell is a T-cell, and wherein the T-cell exhibits one or more of increased T-cell activation, increased T-cell proliferation, decreased T-cell exhaustion, and decreased T-cell tolerance.

274. The modified immune cell of claim 273, wherein increased T-cell activation comprises increased production of a cytokine.

275. The modified immune cell of claim 274, wherein the cytokine comprises one or more selected from the group consisting of IL-2, IFN-γ, TNFα, and GM-CSF.

276. The modified immune cell of any one of claims 273-275, wherein increased T-cell activation comprises increased cell surface expression of one or more T-cell activation markers.

277. The modified immune cell of claim 276, wherein the T-cell activation markers comprise one or more selected from the group consisting of CD25, CD69, and CTLA4.

278. The modified immune cell of any one of claims 273-277, wherein the T-cell has been or is in contact with an anti-CD3 antibody alone or in combination with an anti-CD28 antibody.

279. The modified immune cell of any one of claims 273-277, wherein the T-cell has been or is in contact with IL-2 alone or in combination with an anti-CD3 antibody and/or an anti-CD28 antibody.

280. The modified immune cell of any one of claims 273-272, wherein the modified immune cell is a NK cell, and wherein the NK cell exhibits increased NK cell activation.

281. The modified immune cell of claim 280, wherein increased NK cell activation comprises increased production of a cytokine.

282. The modified immune cell of claim 281, wherein the cytokine comprises one or more selected from the group consisting of IFN-γ, TNFα, and MIP1β.

283. The modified immune cell of any one of claims 270-272, wherein the modified immune cell is a B cell, and wherein the B cell exhibits increased B cell activation, optionally wherein increased B cell activation comprises increased expression of CD69.

284. The modified immune cell of any one of claims 270-283, wherein the modified immune cell is a human immune cell.

285. The modified immune cell of any one of claims 270-284, wherein the modified immune cell comprises a recombinant chimeric receptor.

286. The modified immune cell of claim 285, wherein the recombinant chimeric receptor is a chimeric antigen receptor.

287. A composition comprising a cell population containing the modified immune cell of any one of claims 268-286.

288. The composition of claim 287, further comprising a pharmaceutically acceptable excipient.

289. The composition of claim 287, wherein the composition is in a culture vessel.

290. The composition of claim 289, wherein the culture vessel is a tube, a dish, a bag, a multi well plate or a flask.

291. The composition of claim 287 or claim 288, wherein the composition is in a suitable container.

292. The composition of claim 291, wherein the suitable container is a bottle, a vial, a syringe, an intravenous bag or a tube.

293. A method of modulating the immune response, the method comprising administering an effective amount of the modified immune cell of any one of claims 268-286 or an effective amount of the composition of any one of claims 287-292 to an individual in need thereof.

294. The method of claim 293, wherein the individual has a cancer.

295. A method of treating a cancer responsive to inhibition of Cbl-b activity, the method comprising administering an effective amount of the modified immune cell of any one of claims 268-286 or an effective amount of the composition of any one of claims 287-292 to an individual having the cancer responsive to inhibition of Cbl-b activity.

296. The method of claim 294 or 295 wherein the cancer is a hematologic cancer.

297. The method of claim 296, wherein the hematologic cancer is a lymphoma, a leukemia, or a myeloma.

298. The method of claim 294 or 295 wherein the cancer is a non-hematologic cancer.

299. The method of claim 298, wherein the non-hematologic cancer is a sarcoma or a carcinoma.

300. A method of inhibiting abnormal cell proliferation, the method comprising administering an effective amount of the modified immune cell of any one of claims 268-286 or an effective amount of the composition of any one of claims 287-292 to an individual in need thereof.

301. The method of claim 300, wherein the abnormal cell proliferation is hyperplasia or cancer cell proliferation.

302. The method of claim 301, wherein the cancer cell is from a hematologic cancer.

303. The method of claim 302, wherein the hematologic cancer is a lymphoma, a leukemia, or a myeloma.

304. The method of claim 301, wherein the cancer cells is from a non-hematologic cancer.

305. The method of claim 304, wherein the non-hematologic cancer is a sarcoma or a carcinoma.

306. A method of modulating the immune response, the method comprising administering an effective amount of a Cbl-b inhibitor to an individual to modulate the immune response in the individual, wherein the Cbl-b inhibitor is a compound of Formula (I-A), Formula (I), Formula (II-A), Formula (II), Formula (III), Formula (III-A), or Formula (IV) of any one of claims 1-238.

307. A method of inhibiting Cbl-b activity, the method comprising administering an effective amount of a Cbl-b inhibitor to an individual to inhibit Cbl-b activity in the individual, wherein the Cbl-b inhibitor is a compound of Formula (I-A), Formula (I), Formula (II-A), Formula (II), Formula (III), Formula (III-A), or Formula (IV) of any one of claims 1-238.

308. A method of treating a cancer responsive to inhibition of Cbl-b activity, the method comprising administering an effective amount of a Cbl-b inhibitor to an individual to treat the cancer responsive to inhibition of Cbl-b activity, wherein the Cbl-b inhibitor is a compound of Formula (I-A), Formula (I), Formula (II-A), Formula (II), Formula (III), Formula (III-A), or Formula (IV) of any one of claims 1-238.

309. The method of claim 308, wherein the cancer is a hematologic cancer.

310. The method of claim 298, wherein the hematologic cancer is a lymphoma, a leukemia, or a myeloma.

311. The method of claim 308, wherein the cancer is a non-hematologic cancer, is a sarcoma or a carcinoma.

312. The method of claim 311, wherein the non-hematologic cancer is a sarcoma or a carcinoma.

313. The method of any one of claims 308-312, further comprising administering an effective amount of the modified immune cell of any one of claims 268-286 or an effective amount of the composition of any one of claims 287-292 to the individual to treat the cancer.

314. A method of inhibiting abnormal cell proliferation, the method comprising administering an effective amount of a Cbl-b inhibitor to an individual to inhibit abnormal cell proliferation in the individual, wherein the Cbl-b inhibitor is a compound of Formula (I-A), Formula (I), Formula (II-A), Formula (II), Formula (III), Formula (III-A), or Formula (IV) of any one of claims 1-238.

315. The method of claim 314, wherein the abnormal cell proliferation is hyperplasia or cancer cell proliferation.

316. The method of claim 315, wherein the cancer cell is from a hematologic cancer.

317. The method of claim 316, wherein the hematologic cancer is a lymphoma, a leukemia, or a myeloma.

318. The method of claim 315, wherein the cancer cell is from a non-hematologic cancer.

319. The method of claim 318, wherein the non-hematologic cancer is a sarcoma or a carcinoma.

320. The method of any one of claims 306-319, wherein the individual has one or more of increased T-cell activation, increased T-cell proliferation, decreased T-cell exhaustion, and decreased T-cell tolerance after administration of the Cbl-b inhibitor.

321. The method of claim 320, wherein increased T-cell activation comprises increased production of a cytokine.

322. The method of claim 321, wherein the cytokine comprises one or more selected from the group consisting of IL-2, IFN-γ, TNFα, and GM-CSF.

323. The method of any one of claims 320-322, wherein increased T-cell activation comprises increased cell surface expression of one or more T-cell activation markers.

324. The method of claim 323, wherein the T-cell activation markers comprise one or more selected from the group consisting of CD25, CD69, and CTLA4.

325. The method of any one of claims 306-324, wherein the individual has increased NK cell activation after administration of the Cbl-b inhibitor.

326. The method of claim 325, wherein increased NK cell activation comprises increased production of a cytokine.

327. The method of claim 326, wherein the cytokine comprises one or more selected from the group consisting of IFN-γ, TNFα, and MIP1β.

328. The method of any one of claims 306-327, wherein the individual has increased B cell activation after administration of the Cbl-b inhibitor, optionally wherein increased B cell activation comprises increased expression of CD69.

329. A cell culture composition comprising a cell population containing an immune cell and a Cbl-b inhibitor, wherein the Cbl-b inhibitor is a compound of Formula (I-A), Formula (I), Formula (II-A), Formula (II), Formula (III), Formula (III-A), or Formula (IV) of any one of claims 1-238.

330. The cell culture composition of claim 329, wherein the immune cell is a cell selected from the group consisting of: a hematopoietic cell, a multipotent stem cell, a myeloid progenitor cell, a lymphoid progenitor cell, a T-cell, a B cell, and a NK cell.

331. The cell culture composition of claim 329 or 330, further comprising an anti-CD3 antibody alone or in combination with an anti-CD28 antibody.

332. The cell culture composition of any one of claims 329-331, wherein the immune cell is an engineered immune cell comprising a recombinant chimeric receptor.

333. The cell culture composition of claim 332, wherein the recombinant chimeric receptor is a chimeric antigen receptor.

334. A pharmaceutical composition comprising a Cbl-b inhibitor and one or both of an adjuvant and an antigen, wherein the Cbl-b inhibitor is a compound of Formula (I-A), Formula (I), Formula (II-A), Formula (II), Formula (III), Formula (III-A), or Formula (IV) of any one of claims 1-238.

335. The pharmaceutical composition of claim 334, wherein the antigen is a cancer antigen.

336. An article of manufacture comprising the modified immune cell of any one of claims 268-286, the composition of any one of claims 287-292, the cell culture composition of any one of claims 329-333, or the pharmaceutical composition of any one of claims 124-214.

337. The article of manufacture of claim 336, wherein the modified immune cell or cell culture composition is in a tube, a dish, a bag, a multiwell plate or a flask.

338. The article of manufacture of claim 336, wherein the modified immune cell or pharmaceutical composition is in a bottle, a vial, a syringe, an intravenous bag or a tube.

339. A kit comprising the modified immune cell of any one of claims 268-286 or the composition of any one of claims 287-292.

340. The kit of claim 339, wherein the modified immune cell is in a tube, a dish, a bag, a multiwell plate, or a flask.

341. The kit of claim 339, wherein the modified immune cell is in a bottle, a vial, a syringe, an intravenous bag, or a tube.

342. The kit of any one of claims 339-341, wherein the kit comprises instructions for administering the modified immune cell or composition to an individual according to the method of any one of claims 293-305.

343. A kit comprising the pharmaceutical composition of any one of claims 124-214.

344. The kit of claim 343, wherein the kit comprises instructions for administering the pharmaceutical composition to an individual according to the method of any one of claims 306-308.

345. A kit comprising the cell culture composition of any one of claims 329-333.

346. The kit of claim 345, wherein the kit comprises instructions for producing a modified immune cell according to the method of any one of claims 257-267.

347. A method for treating or preventing a disease or condition associated with Cbl-b activity, the method comprising administering a Cbl-b inhibitor to an individual in need thereof, wherein the Cbl-b inhibitor is a compound of Formula (I-A), Formula (I), Formula (II-A), Formula (II), Formula (III), Formula (III-A), or Formula (IV) of any one of claims 1-238.

348. Use of a Cbl-b inhibitor in the manufacture of a medicament for treating or preventing a disease or condition associated with Cbl-b activity, wherein the Cbl-b inhibitor is a compound of Formula (I-A), Formula (I), Formula (II-A), Formula (II), Formula (III), Formula (III-A), or Formula (IV) of any one of claims 1-238.

349. Use of a Cbl-b inhibitor in the manufacture of a medicament for treating cancer, wherein the Cbl-b inhibitor is a compound of Formula (I-A), Formula (I), Formula (II-A), Formula (II), Formula (III), Formula (III-A), or Formula (IV) of any one of claims 1-238.

350. A Cbl-b inhibitor for use in treating or preventing a disease or condition associated with Cbl-b activity, wherein the Cbl-b inhibitor is a compound of Formula (I-A), Formula (I), Formula (II-A), Formula (II), Formula (III), Formula (III-A), or Formula (IV) of any one of claims 1-238.

351. A Cbl-b inhibitor for use in treating cancer, wherein the Cbl-b inhibitor is a compound of Formula (I-A), Formula (I), Formula (II-A), Formula (II), Formula (III), Formula (III-A), or Formula (IV) of any one of claims 1-238.
